(19) United States
(12) Patent Application Publication
(10) Pub. No.: US 2024/0158392 A1
(43) Pub. Date: May 16, 2024

(54) SMALL MOLECULE INHIBITORS OF SALT INDUCIBLE KINASES

(71) Applicants: Janssen Biotech, Inc., Horsham, PA (US); The Broad Institute, Inc., Cambridge, MA (US); The General Hospital Corporation, Boston, MA (US)

(72) Inventors: Genesis M. BACANI, Horsham, PA (US); Wenying CHAI, Horsham, PA (US); De Michael CHUNG, Horsham, PA (US); Steven D. GOLDBERG, Horsham, PA (US); Gavin HIRST, Horsham, PA (US); Virnedar KAUSHIK, Cambridge, MA (US); Eduardo V. MERCADO-MARIN, Horsham, PA (US); Donald RAYMOND, Cambridge, MA (US); Mark SEIERSTAD, Horsham, PA (US); Russel C. SMITH, Horsham, PA (US); Thomas SUNDBERG, Cambridge, MA (US); Mark S. TICHENOR, Horsham, PA (US); Jennifer D. VENABLE, Horsham, PA (US); Jianmei WEI, Horsham, PA (US); Ramnik XAVIER, Cambridge, MA (US); Helena C. STEFFENS, Horsham, PA (US)

(21) Appl. No.: 18/263,384

(22) PCT Filed: Jan. 31, 2022
(86) PCT No.: PCT/US2022/070431

§ 371 Date: Jul. 28, 2023

Related U.S. Application Data
(60) Provisional application No. 63/144,034, filed on Feb. 1, 2021.

(51) Int. Cl. C07D 471/04; C07D 487/04; C07D 519/00

(52) U.S. Cl. CPC C07D 471/04; C07D 487/04; C07D 519/00

(57) ABSTRACT

Small molecule inhibitors of salt inducible kinases (SIKs) are provided. In particular, compounds of formula (I), and tautomers, stereoisomers, pharmaceutically acceptable salts and solvates thereof are provided. Also provided are pharmaceutical compositions containing the compounds, methods of preparing the compounds, and methods of using the compounds for inhibiting SIKs, such as SIK1 and SIK2, and methods of treating diseases mediated by SIKs.

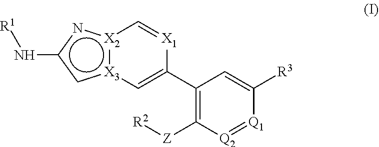

### CROSS REFERENCE TO RELATED APPLICATION

This application claims priority to U.S. Provisional Application No. 63/144,034 filed on Feb. 1, 2021, the disclosure of which is incorporated herein by reference in its entirety.

### FIELD

The present invention relates to the field of chemistry and medicine. More particularly, the present invention relates to salt-inducible kinases inhibitors and their use in medical treatment.

### BACKGROUND

A protein kinase is an enzyme that catalyzes the transfer of phosphate groups to proteins or other organic molecules. The salt-inducible kinases (SIKs) are Ser/Thr kinases members of the Adenosine Monophosphate-Activated Kinase (AMPK) subfamily of kinases and three isoforms have been described (i.e., SIK1, SIK2 (QIK), and SIK3 (QSK) (1). Small molecule protein kinase inhibitors are useful for treating disease including, but not limited to, proliferative diseases (cancer, benign neoplasms, pathological angiogenesis), immune disorders (auto-inflammatory disease, autoimmune disease), and disorders of the musculoskeletal system. For example, the pan-Janus kinase inhibitor tofacitinib is approved to treat rheumatoid arthritis and ulcerative colitis. There is a need for small molecule kinase inhibitors that selectively inhibit SIK1, SIK2 and/or SIK3.

Crohn's disease and ulcerative colitis are the two major forms of inflammatory bowel disease (IBD), a disorder characterized by chronic inflammation of the gastrointestinal (GI) tract. There is an urgent need to discover new therapies for these disorders as broadly immunosuppressive therapies used to treat IBD are not always effective or durable, and can cause severe side effects (2). Aberrant host responses to the intestinal microbiota in genetically susceptible individuals disrupts immune homeostasis, leading to elevated levels of proinflammatory cytokines such as tumor necrosis factor (TNF) and interleukin (IL-12) and the development of IBD. In the gastrointestinal tract, homeostasis is an active process that requires a careful balance of host responses to the enteric luminal contents (3), a process for which the immunoregulatory cytokine IL-10 is vital (4). Single Nucleotide Polymorphisms (SNPs) in the genetic loci containing the anti-inflammatory cytokine IL10 or its receptor (IL10RA) are associated with increased risk of Crohn's disease and ulcerative colitis (5), and loss-of-function mutations in the coding regions of these genes lead to a rare but severe, pediatric-onset enterocolitis in affected individuals. This phenotype is recapitulated in mice deficient in either Il10^{-/-} or Il10ra^{-/-}, which develop spontaneous colitis (4, 6). Together, these genetic data suggest that IBD patients may benefit from therapies that augment IL-10 function specifically in the intestinal mucosa.

Approaches to target IL-10 to inflamed tissues hold therapeutic promise (7). For example, oral administration of bacteria (Lactococcus lactis) engineered to express IL-10 resulted in beneficial, though not persistent, effects in Crohn's disease patients (8). In addition, a fusion protein linking recombinant IL-10 to an antibody targeting an inflammation associated integrin (dekavil) is well tolerated in initial clinical safety trials (9), (10). Similarly, small molecules that promote IL-10 production by aberrantly activated intestinal immune cells may represent a viable approach to dampen intestinal inflammation in IBD patients while avoiding the limitations of systemically administering recombinant IL-10.

Several mechanistic classes of small molecules have been shown to enhance IL-10 production in activated myeloid cells, such as stimulating cAMP Response Element-Binding Protein (CREB) signaling with prostanoid receptor agonists (11), phosphodiesterase (PDE) inhibitors (12-14); disrupting signaling to NF-κB activation with inhibitors of protein kinase C (PKC) or glycogen synthase kinase-3β (GSK-3β) (15, 16); and increasing acetylated α-tubulin levels with paclitaxel or the HDAC6 inhibitor tubastatin A, reported to promote IL-10 production via p38 MAP kinase (17). However, these approaches either do not produce the desired outcome or are associated with potential liabilities (e.g., prostanoid receptor agonist like PGE2 do not show selectivity for IL-10 induction (13), and potent emetic effects have hampered development of PDE inhibitors to treat IBD (18).

Small molecule SIK inhibitors may hold particular therapeutic potential for treatment of IBD because SIK inhibitors both enhance IL-10 production and reduce production of inflammatory cytokines, including TNF and IL-12, in both activated murine dendritic cells and monocyte-derived human macrophages and dendritic cells from healthy volunteers (13), (19). In addition, direct injection of a small molecule SIK inhibitor enhances levels of IL-10 and reduces levels of TNF in the serum and colon of mice during the acute inflammatory response induced by stimulation with lipopolysaccharide (20). The ability of SIK inhibitors to coordinately up-regulate IL-10 and suppress inflammatory cytokines is supported by studies in SIK1 and SIK2 kinase dead knock-in mice; Dendritic cells derived from Sik1KI, Sik2KI double mutant mice secreted elevated levels of IL-10 and diminished levels of TNF and IL-12/23p40 in response to LPS (21). Therefore, small molecule SIK inhibitors represent a potential therapeutic strategy to enhance IL-10 production and concomitantly suppress production of pro-inflammatory cytokines by immune cells in the inflamed intestinal mucosa of IBD patients.

In quiescent myeloid cells, the SIKs phosphorylate the CREB Regulated Transcription Coactivator (CRTC3)-3 as well as Histone Deacetylases (HDAC)-4 and HDAC5, which results in their cytosolic sequestration by binding to 14-3-3 proteins (12, 22, 23). Activation of Protein Kinase A (PKA) following prostanoid receptor stimulation inhibits SIK activity, enabling CRTC3 to enter the nucleus and stimulate transcription of CREB targets like IL-10 (12, 22). In addition, translocation of HDAC4 and HDAC5 into the nucleus in response to small molecule SIK inhibitors has been shown to suppress inflammatory cytokine production by deacetylation of NF-κB subunits and histone lysines near promoters of several pro-inflammatory cytokines (24).

SIK2 is a centrosome kinase and has a role in bipolar mitotic spindle formation in some cancers (25). Elevated levels of SIK2 protein is present in approximately 30% of serous ovarian cancer relative to normal ovarian epithelium tissue (26). Suppressing SIK2 function by depleting SIK2 transcript with targeting siRNAs or inhibiting SIK2 function with a small molecule pan-SIK inhibitor disrupts mitosis of ovarian cancer cell lines and increases sensitivity to paclitaxel in cell culture and xenograft studies (25, 26). Additionally, SIK3 has been identified as a dependency for a subset of acute myeloid leukemias (AMLs) (27). AML lines that are susceptible to SIK3 knockout are driven by mixed lineage leukemia (MLL) fusion oncoproteins, which creates a dependency on the MEF2C transcription factor. SIK3 activity is required to maintain inhibitory phosphorylation on HDAC4 and HDAC5. When SIK3 activity is reduced, HDAC4 and HDAC5 are relieve of inhibitory phosphorylation resulting in suppression of MEF2C function. CRISPR/Cas9-based knockout of SIK3 or inhibiting SIK3 function with a small molecule pan-SIK inhibitor suppresses proliferation of MEF2C-dependent AMLs (27).

### BRIEF SUMMARY

Accordingly, there is a need in the art for novel inhibitors of salt inducible kinases (SIKs). Small molecule inhibitors of SIKs, such as SIK1 and SIK2, are provided.

In a general aspect, provided are small molecule inhibitors of salt inducible kinases (SIKs).

In an embodiment, provided is a compound of formula (I):

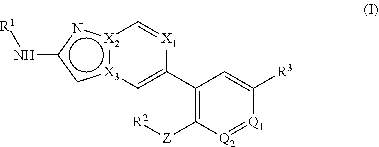

or a stereoisomer, tautomer, pharmaceutically acceptable salt or solvate thereof,

wherein:
X₁, X₂, and X₃ are independently N or CH;
Q₁ and Q₂ are independently N or CH;
Z is —O—Cₙ₁H₂ₙ₁—, or —NR⁶—(CH₂)ₙ₂—, provided that O or N is directly bonded to the pyridinyl ring or phenyl ring or pyridazinyl ring;
n1 is 0, 1 or 2, and 2n1 is twice of n1;
n2 is 0 or 1;
R¹ is —C(O)R⁵, phenyl, a 5- to 6-membered heteroaryl having 1-3 nitrogen heteroatoms, or

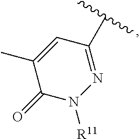

wherein each of the phenyl and 5- to 6-membered heteroaryl is optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino, wherein the 5- to 6-membered heteroaryl is optionally fused to a ring selected from the group consisting of benzene, piperidine, piperazine, and a 5- to 6-membered heteroaryl having 1-2 nitrogen heteroatoms, wherein the 5- to 6-membered heteroaryl is optionally substituted with one or more —CH₃;
R⁵ is cyclopropyl optionally substituted with one or more substituents independently selected from the group consisting of —F, —CH₃, C₁ fluoroalkyl, and phenyl;
R³ is hydrogen, —CH₃, —CH₂CH₃, propynyl, C₁ fluoroalkyl, —CH₂OCH₃, —COOCH₂CH₃, —CN, —Cl, —N(R¹⁰)₂, or —OR¹⁵;
R² is:
   a 4- to 9-membered monocyclic or bicyclic heterocyclyl having 1 to 3 heteroatoms independently selected from oxygen and nitrogen, wherein the 4- to 9-membered monocyclic or bicyclic heterocyclyl is optionally substituted with one or more substituents independently selected from the group consisting of C₁₋₃ alkyl, C₁₋₂ fluoroalkyl, C₁₋₄ hydroxyalkyl, —CD₃, —F, —OH, —OCH₃, oxo, cyclopropyl, —COOR⁸, —CONH₂, —C(O)CH₃, —N(R¹⁰)₂, —(CH₂)ₘOCH₃, —CN, —CH₂CN, —CH₂(C₅H₄N), —SO₂CH₃, and phenyl,
   a C₃-C₆ cycloalkyl, wherein the C₃-C₆ cycloalkyl is optionally substituted with one or more substituents independently selected from the group consisting of —CH₃, —F, —OH, —OCH₃, —N(R¹²)₂, —CH₂OH, —═O, phenyl, —CN, C₁ fluoroalkyl, —CONH₂, ═NH(OH), —SO₂CH₃, and —CH(CH₃)₂,
   a C₁₋₅ alkyl, wherein the C₁₋₅ alkyl is optionally substituted with one or more substituents independently selected from the group consisting of —OH, —N(R¹⁰)₂, C₁ fluoroalkyl, —COOR₈, —CON(R⁸)₂, —CH₃, C₃-C₆ cycloalkyl, phenyl, pyridyl, —SO₂CH₃, and —CN, wherein the C₃-C₆ cycloalkyl is optionally substituted with one or more —CH₃,
   a 5- to 6-membered heteroaryl having 1 to 2 nitrogen atoms, wherein the 5- to 6-membered heteroaryl is optionally substituted with one or more substituents independently selected from the group consisting of —CH₃ and C₁ fluoroalkyl,
   or phenyl, or phenyl substituted with —OCHF₂,
R⁶ is hydrogen,
or alternatively, when Z is —NR⁶—(CH₂)ₙ₂— and n2 is 0, R² and R⁶ are taken together with the nitrogen atom to which they are attached to form a 4- to 8-membered monocyclic or bicyclic heterocyclyl having 1-2 nitrogen heteroatoms, wherein the 4- to 8-membered monocyclic or bicyclic heterocyclyl is optionally substituted with one or more substituents independently selected from the group consisting of —OH, C₁ fluoroalkyl, C₁₋₃ hydroxyalkyl, —C(O)N(R¹⁰)₂ and —CH₂N(R¹⁰)₂;
each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN;
each R⁹ is independently C₁ fluoroalkyl or —CH₃;
each R¹⁰ is independently hydrogen, —COCH₃, —SO₂CH₃, C₁₋₂ alkyl, or C₁₋₂ fluoroalkyl;
each R¹¹ is independently hydrogen, C₁₋₅ alkyl, —CH₂CH₂—OCHF₂, cyclopropyl, or —CH₂-cyclopropyl; and
each R¹² is independently hydrogen, —CH₃, C₃H₅, —SO₂CH₃, —CH₂CN, —CH₂(C₆H₅), —CH₂COOCH₂CH₃, or a 5- to 6-membered heteroaryl having 1 to 2 nitrogen atoms;
each R¹³ is independently hydrogen or C₁₋₄ alkyl;
each R¹⁴ is independently cyclopropyl or C₁₋₄ alkyl;
each R¹⁵ is independently hydrogen or C₁₋₄ alkyl, wherein the C₁₋₄ alkyl is optionally substituted with one or more substituents independently selected from the group consisting of —OH, —F, and —CN; and
m is 1 or 2.

In some embodiments, provided is a compound of formula (I), or a stereoisomer, tautomer, pharmaceutically acceptable salt or solvate thereof, wherein R¹ is —C(O)R⁵ and R⁵ is cyclopropyl.

In some embodiments, provided is a compound of formula (I), or a stereoisomer, tautomer, pharmaceutically acceptable salt or solvate thereof, wherein R¹ is:
triazolyl, pyrazolyl, pyrazinyl, pyridazinyl, pyrimidinyl, pyridinyl, pyrrolo[2,3-b]pyridinyl, imidazo[1,2-b]pyridazinyl, indazolyl, 4,5,6,7-tetrahydropyrazolo[1,5-a]pyrazinyl, 5,6,7,8-tetrahydro-2,7-naphthyridinyl, or 5,6,7,8-tetrahydro-2,6-naphthyridinyl, wherein each of the triazolyl, pyrazolyl, pyrazinyl, pyridazinyl, pyrimidinyl, pyrrolo[2,3-b]pyridinyl, imidazo[1,2-b]pyridazinyl, indazolyl, 4,5,6,7-tetrahydropyrazolo[1,5-a]pyrazinyl, 5,6,7,8-tetrahydro-2,7-naphthyridinyl, 5,6,7,8-tetrahydro-2,6-naphthyridinyl, and pyridinyl is optionally substituted with one or more substituents independently selected from the group consisting of —C(O)NHCH₃, —F, —CH₃, —CH₂CH₃, —NHCH₃, —CHF₂, —C(O)CF₃, —CH₂C(OH)(CH₃)₂, —CH(CH₃)₂, C(CH₃)₃, —C₃H₅, —C(O)N(CH₃)₂, —CH₂OCH₃, —OCHF₂, —CH(OH)CHCF₃, —CO₂H, —CO₂CH₃, —SO₂CH₃, —SO₂N(CH₃)₂,

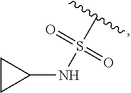 [SMILES: *S(=O)(=O)NC1CC1]

cyclopropyl, pyrrolidinyl, morpholino, and morpholinomethyl;

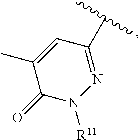

wherein R¹¹ is hydrogen, —CH₃, —CH₂CH₃, —OCHF₂, cyclopropyl, or —CH₂-cyclopropyl; or
phenyl, wherein the phenyl is optionally substituted with one or more substituents independently selected from the group consisting of —COOCH₃, —C(O)N(H)CH₂CH₃, —C(O)N(H)CH₂CF₃, —CO₂CH₃, —OCH₃, —OCHF₂,

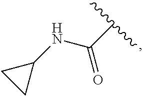 [SMILES: *C(=O)NC1CC1]

—SO₂N(CH₃)₂, and

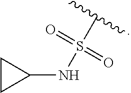 [SMILES: *S(=O)(=O)NC1CC1]

In some embodiments, provided is a compound of formula (I), or a stereoisomer, tautomer, pharmaceutically acceptable salt or solvate thereof, wherein R¹ is:

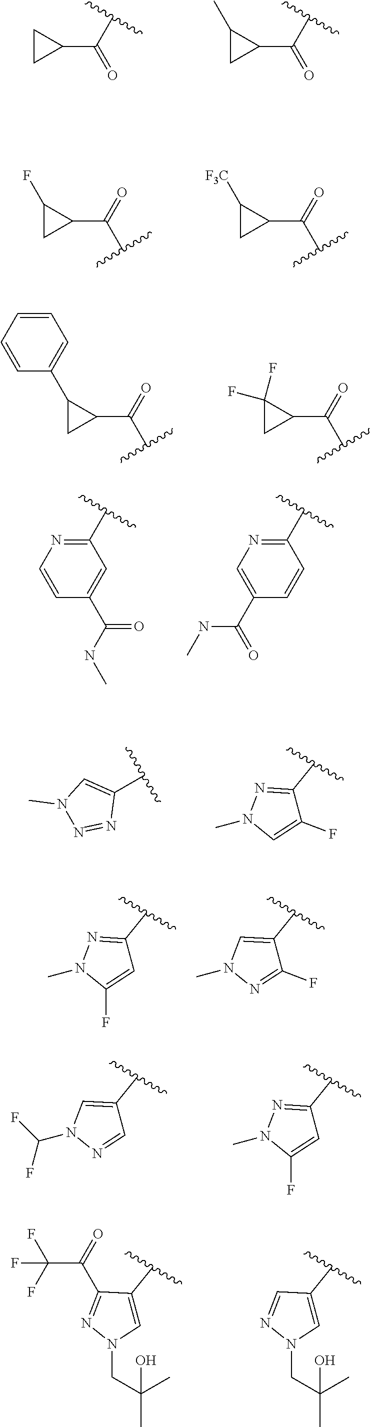

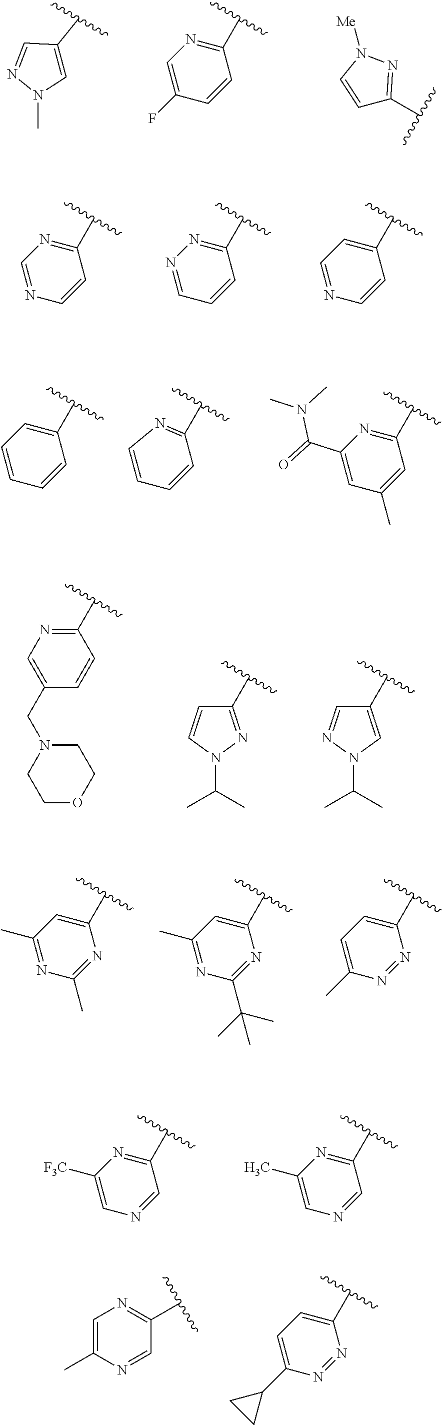

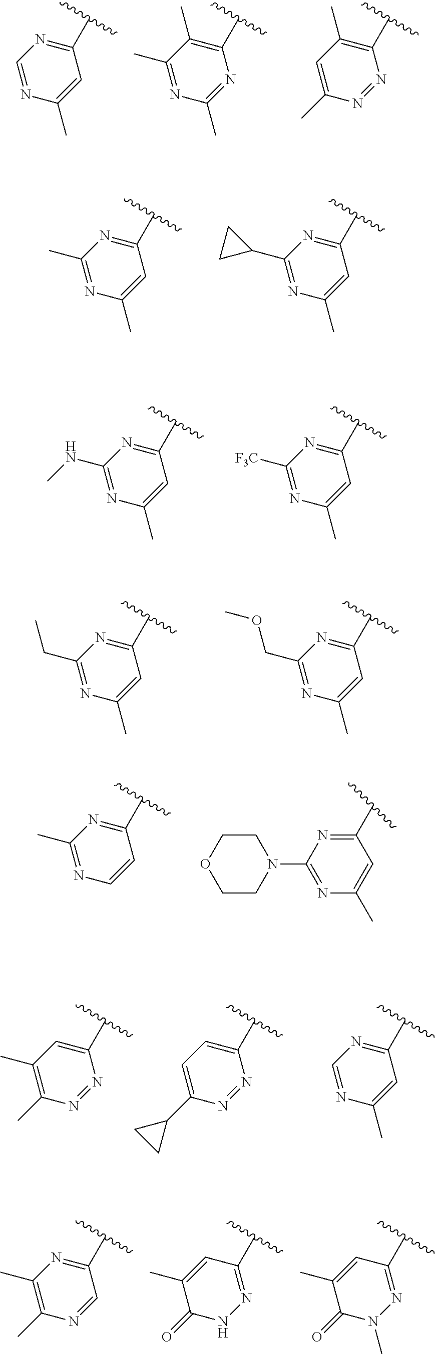

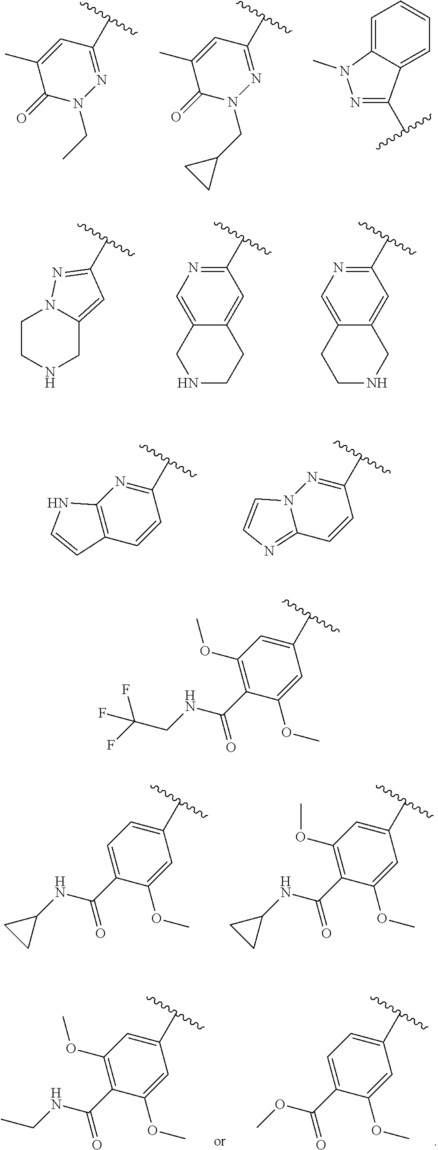

In some embodiments, provided is a compound of formula (I), or a stereoisomer, tautomer, pharmaceutically acceptable salt or solvate thereof, wherein Z is —O—, —O—CH₂—, —O—CH(CH₃)—, or —O—CH₂—CH₂—.

In some embodiments, provided is a compound of formula (I), or a stereoisomer, tautomer, pharmaceutically acceptable salt or solvate thereof, wherein R² is a 4- to 7-membered monocyclic heterocyclyl selected from the group consisting of pyrrolidinyl, piperidinyl, oxazepanyl, morpholinyl, azetidinyl, tetrahydrofuryl, tetrahydropyranyl, oxetanyl, and 1,1-dioxo-1,2,5-thiadiazolidinyl, wherein the 4- to 7-membered monocyclic heterocyclyl is optionally substituted with one or more substituents independently selected from the group consisting of —CH₃, —CD₃, —OCH₃, —CH₂CH₃, —CH(CH₃)₂, —CH₂CF₃, —CF₃, —CH₂OH, —CH₂C(OH)(CH₃)₂, —F, —OH, —NH₂, —CN, oxo, cyclopropyl, —COOC(CH₃)₃, —CONH₂, —C(O)CH₃, —CH₂CN, —CH₂CH₂OCH₃, —CH₂(C₅H₄N), —SO₂CH₃, and phenyl.

In some embodiments, provided is a compound of formula (I), or a stereoisomer, tautomer, pharmaceutically acceptable salt or solvate thereof, wherein R² is a 6- to 9-membered bicyclic heterocyclyl selected from the group consisting of 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonanyl, 6-oxaspiro[3.4]octanyl, and 1-oxaspiro[4.4]nonanyl, wherein the 6- to 9-membered bicyclic heterocyclyl is optionally substituted with one or more substituents independently selected from the group consisting of —CH₃, —CD₃, —CH₂CH₃, —CH(CH₃)₂, —CH₂C(OH)(CH₃)₂, —COOC(CH₃)₃, —CN, —SO₂CH₃, oxo,

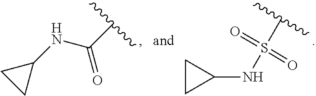 [SMILES: *C(=O)NC1CC1 ; *S(=O)(=O)NC1CC1]

In some embodiments, provided is a compound of formula (I), or a stereoisomer, tautomer, pharmaceutically acceptable salt or solvate thereof, wherein R² is a C₃-C₆ cycloalkyl, wherein the C₃-C₆ cycloalkyl is optionally substituted with one or more substituents independently selected from the group consisting of —NH₂, —OH, —OCH₃, —CH₂OH, ═O, —CONH₂, phenyl, —CH₃, —CF₃, F, —NHCH₃, —NH(C₃H₅), —NHCH₂CN, —NH—COOCH₂CH₃, —NH(C₅H₄N), —NH(C₆H₅),

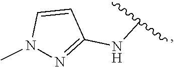 [SMILES: *Nc1ccn(C)n1]

═NH(OH), —SO₂CH₃, and —CH(CH₃)₂.

In some embodiments, provided is a compound of formula (I), or a stereoisomer, tautomer, pharmaceutically acceptable salt or solvate thereof, wherein R² is a C₁₋₅ alkyl optionally substituted with one or more substituents independently selected from the group consisting of —N(CH₃)₂, —OH, —CF₃, —NH₂, —COOC(CH₃)₃, —NH—COCH₃, —NH—SO₂CH₃, —COONH₂, —COON(CH₃)₂, cyclopropyl, methylcyclopropyl, —CH₃, C₃-C₆ cycloalkyl, —SO₂CH₃, phenyl, pyridyl, and —CN.

In some embodiments, provided is a compound of formula (I), or a stereoisomer, tautomer, pharmaceutically acceptable salt or solvate thereof, R² is pyridinyl or pyrazolyl, wherein each of the pyridinyl and pyrazolyl is optionally substituted with one or more substituents independently selected from the group consisting of —CF₃ and —CH₃.

In some embodiments, provided is a compound of formula (I), or a stereoisomer, tautomer, pharmaceutically acceptable salt or solvate thereof, wherein:
Z is —NR⁶—(CH₂)ₙ₂;
n2 is 0; and
R² and R⁶ are taken together to form a 4- to 8-membered monocyclic or bicyclic heterocyclyl selected from the group consisting of:

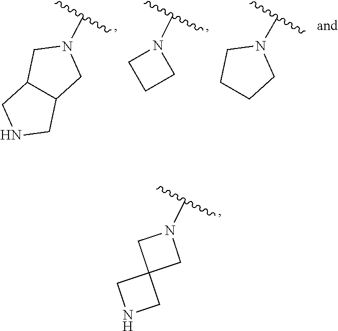 [SMILES: *N1CC2CNCC2C1 ; *N1CCC1 ; *N1CCCC1 ; *N1CC2(CNC2)C1]

wherein the 4- to 8-membered monocyclic or bicyclic heterocyclyl is optionally substituted with one or more substituents independently selected from the group consisting —OH, —CH₂OH, —CH₂N(CH₃)₂, —C(CH₃)₂OH, —CF₃, and —C(O)NHCH₃.

In some embodiments, the compound of formula (I) is a compound of formula (I-A):

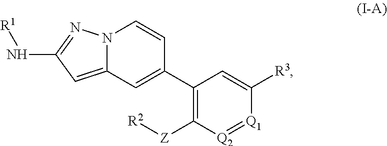

or a stereoisomer, tautomer, pharmaceutically acceptable salt or solvate thereof, wherein Z, Q₁, Q₂, R¹, R², and R³ are defined above as in formula (I).

In some embodiments, the compound of formula (I) is a compound of formula (I-B):

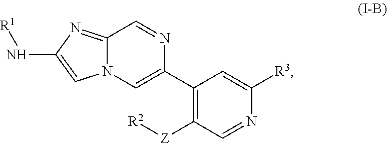

or a stereoisomer, tautomer, pharmaceutically acceptable salt or solvate thereof, wherein Z, R¹, R², and R³ are defined above as in formula (I).

In particular embodiments, provided is a compound selected from the compounds of Examples 1-756 herein, or a stereoisomer, tautomer, pharmaceutically acceptable salt or solvate thereof.

In another aspect, provided is a pharmaceutical composition comprising a compound as describe herein or a stereoisomer, tautomer, pharmaceutically acceptable salt or solvate thereof, and a pharmaceutically acceptable carrier.

In another aspect, provided is a method of inhibiting a salt inducible kinase (SIK) in a subject in need thereof, the method comprising administering to the subject a pharmaceutical composition as described herein. In certain embodiment, the subject is in need of a treatment of a disease, disorder, or condition mediated by a SIK, such as an autoimmune disorder or a proliferative disorder.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the disclosure. Such discussion is not an admission that any or all of these matters form part of the prior art with respect to the disclosure.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. For example, the phrase “at least A, B, and C” means that each of A, B, and C is present. The term “at least one of” preceding a series of elements is to be understood to refer to a single element in the series or any combination of two or more elements in the series. For example, the phrase “at least one of A, B, and C” means that only A is present, only B is present, only C is present, both A and B are present, both A and C are present, both B and C are present, or each of A, B, and C is present. Depending on the context, “at least one of” preceding a series of elements can also encompass situations in which any one or more of the elements is present in greater than one instance, e.g., “at least one of A, B, and C” can also encompass situations in which A is present in duplicate alone or further in combination with any one or more of elements B and C.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, the recitation of “10-fold” includes 9-fold and 11-fold. As used herein, the use of a numerical range expressly includes all possible subranges, all individual numerical values within that range, including integers within such ranges and fractions of the values unless the context clearly indicates otherwise.

As used herein, “subject” means any animal, such as a mammal, to whom will be or has been treated by a method described herein. The term “mammal” as used herein, encompasses any mammal. Examples of mammals include, but are not limited to, cows, horses, sheep, pigs, cats, dogs, mice, rats, rabbits, guinea pigs, and non-human primates (NHPs), such as monkeys or apes, humans, etc.

The phrase “pharmaceutically acceptable salt(s)” means those salts of a compound of interest that are safe and effective for topical use in mammals and that possess the desired biological activity. Pharmaceutically acceptable salts include salts of acidic or basic groups present in the specified compounds. Pharmaceutically acceptable acid addition salts include, but are not limited to, hydrochloride, hydrobromide, hydroiodide, nitrate, sulfate, bisulfate, phosphate, acid phosphate, isonicotinate, carbonate, bicarbonate, acetate, lactate, salicylate, citrate, tartrate, propionate, butyrate, pyruvate, oxalate, malonate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Certain compounds used in the application can form pharmaceutically acceptable salts with various amino acids. Suitable base salts include, but are not limited to, aluminum, calcium, lithium, magnesium, potassium, sodium, zinc, bismuth, and diethanolamine salts. For a review on pharmaceutically acceptable salts see Berge et al., 66 J. Pharm. Sci. 1-19 (1977), incorporated herein by reference.

As used herein, the term “alkyl” means a saturated, monovalent, unbranched or branched hydrocarbon chain. An alkyl group can be unsubstituted or substituted with one or more suitable substituents. Examples of alkyl groups include, but are not limited to, methyl (Me), ethyl (Et), propyl (e.g., n-propyl, isopropyl), butyl (e.g., n-butyl, isobutyl, tert-butyl), and pentyl (e.g., n-pentyl, isopentyl, neopentyl), etc. An alkyl group can have a specified number of carbon atoms. When numbers appear in a subscript after the symbol “C”, the subscript defines with more specificity the number of carbon atoms which that particular alkyl can contain. For example, “C₁ to C₁₀ alkyl” or “C₁₋₁₀ alkyl” is intended to include alkyl groups having 1, 2, 3, 4, 5, 6, 7, 8, 9, and 10 carbon atoms. Additionally, for example, “C₁ to C₄ alkyl” or “C₁₋₄ alkyl” denotes an alkyl having 1, 2, 3, or 4 carbon atoms.

The term “cycloalkyl” refers to any stable monocyclic or polycyclic saturated hydrocarbon ring system. A cycloalkyl group can be unsubstituted or substituted with one or more suitable substituents. A cycloalkyl group can have a specified number of carbon atoms. For example, “C₃ to C₆ cycloalkyl” or “C₃₋₆ cycloalkyl” includes cycloalkyl groups having 3, 4, 5, or 6 ring carbon atoms, i.e., cyclopropyl, cyclobutyl, cyclopentyl, and cyclohexyl. Polycyclic cycloalkyls include bridged, fused, and spiro ring structures in which all ring atoms are carbon atoms. A “spiro ring” is a polycyclic ring system in which two rings share one carbon atom, referred to as the “spiro atom,” which is typically a quaternary carbon atom. A “fused ring” is a polycyclic ring system in which two rings share two adjacent atoms, referred to as “bridgehead atoms,” i.e., the two rings share one covalent bond such that the bridgehead atoms are directly connected. A “bridged ring” is a polycyclic ring system in which two rings share three or more atoms separating the bridgehead atoms by a bridge containing at least one atom. Examples of cycloalkyl groups include, but are not limited to, cyclopropyl, cyclobutyl, cyclopentyl, cyclohexyl, cycloheptyl, cyclooctyl, etc.

The term “aryl” as used herein is a group that contains any carbon-based aromatic group including, but not limited to, phenyl, naphthyl, anthracenyl, phenanthranyl, and the like. Aryl moieties are well known and described, for example, in Lewis, R. J., ed., Hawley's Condensed Chemical Dictionary, 13th Edition, John Wiley & Sons, Inc., New York (1997). An aryl group can be substituted or unsubstituted with one or more suitable substituents. An aryl group can comprise a single ring structure (i.e., monocyclic) or multiple ring structures (i.e., polycyclic, e.g., bicyclic or tricyclic). For example, an aryl group can be a monocyclic aryl group, e.g., phenyl.

The term “heterocyclyl” includes stable monocyclic and polycyclic hydrocarbons that contain at least one heteroatom ring member, such as sulfur, oxygen, or nitrogen, wherein the ring structure is saturated or partially unsaturated, provided the ring system is not fully aromatic. A heterocyclyl group can be unsubstituted, or substituted with one or more suitable substituents at any one or more of the carbon atom(s) and/or nitrogen heteroatom(s) of the heterocyclyl. A heterocyclyl can comprise a single ring structure (i.e., monocyclic) or multiple ring structures (i.e., polycyclic, e.g., bicyclic). Polycyclic heterocyclyls include bridged, fused, and spiro ring structures in which at least one ring atom of at least one of the rings of the polycyclic ring system is a heteroatom, for instance oxygen, nitrogen, or sulfur, wherein bridged, fused, and spiro rings are as defined above. A heterocyclyl ring can be attached to the parent molecule at any suitable heteroatom (typically nitrogen) or carbon atom of the ring. The term “4- to 9-membered monocyclic or bicyclic heterocyclyl” includes any four, five, six, seven, eight, or nine membered monocyclic or bicyclic ring structure containing at least one heteroatom ring member selected from oxygen, nitrogen, and sulfur, or independently selected from oxygen and nitrogen, optionally containing one to three additional heteroatoms independently selected from oxygen, nitrogen, and sulfur, or independently selected from oxygen and nitrogen, wherein the ring structure is saturated or partially unsaturated, provided the ring structure is not fully aromatic.

In certain embodiments, the term “heterocyclyl” refers to 4-, 5-, 6-, or 7-membered monocyclic groups and 6-, 7-, 8-, or 9-membered bicyclic groups which have at least one heteroatom (O, S, or N) in at least one of the rings, wherein the heteroatom-containing ring(s) typically has 1, 2, or 3 heteroatoms, such as 1 or 2 heteroatoms, independently selected from O, S, and/or N, or independently selected from O and N. Examples of monocyclic heterocyclyl groups include, but are not limited to azetidinyl, oxetanyl, tetrahydrofuranyl, pyrrolidinyl, imidazolidinyl, pyrazolidinyl, oxazolidinyl, isoxazolidinyl, thiazolidinyl, isothiazolidinyl, dioxolanyl, dithiolanyl, piperidinyl, piperazinyl, dioxanyl, morpholinyl, azepanyl, oxepanyl, oxazepanyl (e.g., 1,4-oxazepanyl, 1,2-oxazepanyl) and the like. Examples of bicyclic heterocyclyl groups include, but are not limited to, 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, and 5-azaspiro[2.3]hexanyl and the like.

As used herein, the term “heteroaryl” includes stable monocyclic and polycyclic aromatic hydrocarbons that contain at least one heteroatom ring member such as sulfur, oxygen, or nitrogen. A heteroaryl group can be unsubstituted or substituted with one or more suitable substituents. A heteroaryl can comprise a single ring structure (i.e., monocyclic) or multiple ring structures (i.e., polycyclic, e.g., bicyclic or tricyclic). Each ring of a heteroaryl group containing a heteroatom can contain one or two oxygen or sulfur atoms and/or from one to four nitrogen atoms provided that the total number of heteroatoms in each ring is four or less and each ring has at least one carbon atom. Heteroaryl groups which are polycyclic, e.g., bicyclic or tricyclic must include at least one fully aromatic ring, but the other fused ring or rings can be aromatic or non-aromatic. For example, for a bicyclic heteroaryl, the fused rings completing the bicyclic group can contain only carbon atoms and can be saturated, partially saturated, or unsaturated. A heteroaryl can be attached to the parent molecule at any available nitrogen or carbon atom of any ring of the heteroaryl group. In some embodiments, the term “heteroaryl” refers to 5- or 6-membered monocyclic groups and 9- or 10-membered bicyclic groups which have at least one heteroatom (O, S, or N) in at least one of the rings, wherein the heteroatom-containing ring typically has 1, 2, or 3 heteroatoms, such as 1 or 2 heteroatoms, selected from O, S, and/or N. A heteroaryl group can be unsubstituted or substituted with one or more suitable substituents at any one or more of the carbon atom(s) and/or nitrogen heteroatom(s) of the heteroaryl. The nitrogen and sulfur heteroatom(s) of a heteroaryl can optionally be oxidized (i.e., N→O and S(O)r, wherein r is 0, 1 or 2).

Exemplary monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl. Exemplary bicyclic heteroaryl groups include, but are not limited to, indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridinyl, furopyridinyl, dihydroisoindolyl, and tetrahydroquinolinyl.

The term “alkoxy” as used herein refers to an —O-alkyl group, wherein alkyl is as defined above. An alkoxy group is attached to the parent molecule through a bond to an oxygen atom. An alkoxy group can have a specified number of carbon atoms. For example, “C₁ to C₁₀ alkoxy” or “C₁₋₁₀ alkoxy” is intended to include alkoxy groups having 1, 2, 3, 4, 5, 6, 7, 8, 9, and 10 carbon atoms. Additionally, for example, “C₁ to C₄ alkoxy” or “C₁₋₄ alkoxy” denotes an alkoxy having 1, 2, 3, or 4 carbon atoms. Examples of alkoxy include, but are not limited to, methoxy, ethoxy, propoxy (e.g., n-propoxy, isopropoxy), butoxy (e.g., n-butoxy, isobutoxy, tert-butoxy), pentyloxy (e.g., n-pentyloxy, isopentyloxy, neopentyloxy), etc. An alkoxy group can be unsubstituted or substituted with one or more suitable substituents. Similarly, “alkylthio” or “thioalkoxy” represents an alkyl group as defined above attached to the parent molecule through a bond to a sulfur atom, for example, —S-methyl, —S-ethyl, etc. Representative examples of alkylthio include, but are not limited to, —SCH₃, —SCH₂CH₃, etc.

As used herein, the term “halogen” means fluorine, chlorine, bromine, or iodine. Correspondingly, the term “halo” means fluoro, chloro, bromo, and iodo.

“Haloalkyl” is intended to include both branched and straight-chain saturated aliphatic hydrocarbon radicals substituted with one or more halogen atoms. “Fluorinated alkyl” or “fluoroalkyl” in particular refers to any alkyl group as defined above substituted with at least one fluoro atom, e.g., one to three fluoro atoms, such as one, two, or three fluoroatoms. Examples of haloalkyl include, but are not limited to, fluoromethyl, difluoromethyl, trifluoromethyl, chloromethyl, dichloromethyl, trichloromethyl, pentafluoroethyl, pentachloroethyl, 2,2,2-trifluoroethyl, heptafluoropropyl, and heptachloropropyl. Suitable examples of fluoroalkyl in particular include, but are not limited to, —CF₃, —CHF₂, —CH₂F, —CH₂CF₃, —CF₂CF₃, and the like.

The terms “hydroxy” and “hydroxyl” can be used interchangeably, and refer to —OH.

The term “carboxy” refers to —COOH.

The term “ester” refers to —COOR, wherein R is alkyl as defined above.

The term “cyano” refers to —CN.

The term “oxo” refers to a double bonded oxygen group, i.e., a substituent group of the formula ═O.

The term “keto” refers to —C(O)R, wherein R is alkyl as defined above.

The term “amino” refers to —NH₂. One or more hydrogen atoms of an amino group can be replaced by a substituent such as an alkyl group, which is referred to as an “alkylamino.” Alkylamino groups have one or both hydrogen atoms of an amino group replaced with an alkyl group and is attached to the parent molecule through a bond to the nitrogen atom of the alkylamino group. For example, alkylamino includes methylamino (—NHCH₃), dimethylamino (—N(CH₃)₂), —NHCH₂CH₃ and the like.

The term “aminoalkyl” as used herein is intended to include both branched and straight-chain saturated aliphatic hydrocarbon groups substituted with one or more amino groups. For example, “C₁₋₄ aminoalkyl” is intended to include alkyl groups having 1, 2, 3, or 4 carbon atoms substituted with one or more amino groups. Aminoalkyl groups are attached to the parent molecule through a bond to a carbon atom of the alkyl moiety of the aminoalkyl group. Representative examples of aminoalkyl groups include, but are not limited to, —CH₂NH₂, —CH₂CH₂NH₂, and —CH₂CH(NH₂)CH₃.

As used herein, “amido” refers to —C(O)N(R)₂, wherein each R is independently an alkyl group (including both branched and straight-chain alkyl groups) or a hydrogen atom. Examples of amido groups include, but are not limited to, —C(O)NH₂, —C(O)NHCH₃, and —C(O)N(CH₃)₂.

The terms “hydroxylalkyl” and “hydroxyalkyl” are used interchangeably, and refer to a branched or straight-chain aliphatic hydrocarbon group substituted with one or more hydroxyl groups. Hydroxyalkyl groups are attached to the parent molecule through a bond to a carbon atom of the alkyl moiety of the hydroxyalkyl group. A hydroxyalkyl group can have a specified number of carbon atoms. For example, “C₁ to C₁₀ hydroxyalkyl” or “C₁₋₁₀ hydroxyalkyl” is intended to include hydroxyalkyl groups having 1, 2, 3, 4, 5, 6, 7, 8, 9, and 10 carbon atoms. Additionally, for example, “C₁ to C₄ hydroxylalkyl” or “C₁₋₄ hydroxyalkyl” denotes a hydroxyalkyl group having 1, 2, 3, or 4 carbon atoms. Examples of hydroxyalkyl include, but are not limited to, hydroxylmethyl (—CH₂OH), hydroxylethyl (—CH₂CH₂OH), etc.

In accordance with convention used in the art:

[symbol]

is used in structural formulas herein to depict the bond that is the point of attachment of a group, moiety or substituent to the core, backbone, or parent molecule structure.

When a bond to a substituent is shown to cross a bond connecting two atoms in a ring, then such substituent can be bonded to any atom on the ring.

The term “substituted” as used herein with respect to any organic radical (e.g., alkyl, cycloalkyl, heteroaryl, aryl, heterocyclyl, etc.) means that at least one hydrogen atom is replaced with a non-hydrogen group, provided that all normal valencies are maintained and that the substitution results in a stable compound. When a particular group is “substituted,” that group can have one or more substituents, such as from one to five substituents, one to three substituents, or one to two substituents, independently selected from the list of substituents. The term “independently” when used in reference to substituents, means that when more than one of such substituents is possible, such substituents can be the same or different from each other. Examples of suitable substituents include, but are not limited to, alkyl, halo, haloalkyl, alkoxy, amido, hydroxy, hydroxyalkyl, amino, carboxyl, ester, oxo, cyano and the like.

When any variable occurs more than one time in any constituent or formula for a compound, its definition at each occurrence is independent of its definition at every other occurrence. Thus, for example, if a group is shown to be substituted with 0-3 R groups, then said group can be optionally substituted with up to three R groups, and at each occurrence, R is selected independently from the definition of R.

The terms “optional” or “optionally” mean that the event or circumstance described subsequently can, but need not, occur, and such a description includes the situation in which the event or circumstance does or does not occur. For example, “optionally substituted heterocyclyl” means that a substituent group can be, but need not be, present, and such a description includes the situation of the heterocyclyl group being substituted by a suitable substituent and the heterocyclyl group not being substituted by any substituent.

One skilled in the art will recognize that in certain embodiments compounds described herein can have one or more asymmetric carbon atoms in their structure. As used herein, any chemical formulas with bonds shown only as solid lines and not as solid wedged or hashed wedged bonds, or otherwise indicated as having a particular configuration (e.g., R or S) around one or more atoms, contemplates each possible stereoisomer, or mixture of two or more stereoisomers. Stereoisomers includes enantiomers and diastereomers. Enantiomers are stereoisomers that are non-super-imposable mirror images of each other. A 1:1 mixture of a pair of enantiomers is a racemate or racemic mixture. Diastereomers (or diastereoisomers) are stereoisomers that are not enantiomers, i.e., they are not related as mirror images, and occur when two or more stereoisomers of a compound have different configurations at one or more of the equivalent stereocenters and are not mirror images of each other. Substituent groups (e.g., alkyl, heterocyclyl, etc.) can contain stereocenters in either the R or S configuration.

Thus, included within the scope of the application are the stereochemically pure isomeric forms of the compounds described herein (i.e., a single enantiomer or a single diastereomer) as well as mixtures thereof including their racemates. When a specific stereoisomer is identified, this means that the stereoisomer is substantially free, i.e., associated with less than 50%, less than 20%, less than 5%, and in particular less than 2% or less than 1% of the other stereoisomers. For example, when a compound is for instance specified as (R), this means that the compound is substantially free of the (S) isomer. Compounds described herein can be used as racemic mixtures, enantiomerically or diastereomerically enriched mixtures, or as enantiomerically or diastereomerically pure individual stereoisomers.

Certain examples contain chemical structures that comprise (*R) or (*S) terminology. When (*R) or (*S) is used in the name of a compound or in the chemical representation of the compound, it is intended to mean that the compound is a single isomer at that stereocenter, however absolute configuration of that stereocenter has not been established. Thus, a compound designated as (*R) refers to a compound that is a single isomer at that stereocenter with an absolute configuration of either (R) or (S). A compound designated as (*S) refers to a compound that is a single isomer at that stereocenter with an absolute configuration of either (R) or (S). In cases where the absolute stereochemistry has been established, the structures are named using (R) or (S). The use of the term (R,S) or “racemic” or “rac” in the name of the compound indicates that the compound is a racemate.

Stereochemically pure isomeric forms can be obtained by techniques known in the art in view of the present disclosure. For example, diastereoisomers can be separated by physical separation methods such as fractional crystallization and chromatographic techniques, and enantiomers can be separated from each other by the selective crystallization of the diastereomeric salts with optically active acids or bases or by chiral chromatography. Pure stereoisomers can also be prepared synthetically from appropriate stereochemically pure starting materials, or by using stereoselective reactions.

Compounds described herein can also form tautomers. The term “tautomer” refers to compounds that are interchangeable forms of a particular compound structure and that vary in the displacement of hydrogen atoms and electrons. Tautomers are constitutional isomers of chemical compounds that readily interconvert, usually resulting in relocation of a proton (hydrogen). Thus, two structures can be in equilibrium through the movement of pi electrons and an atom (usually hydrogen). All tautomeric forms and mixtures of tautomers of the compounds described herein are included with the scope of the application.

Compounds described herein can exist in solvated and unsolvated forms. The term “solvate” means a physical association, e.g., by hydrogen bonding, of a compound of the application with one or more solvent molecules. The solvent molecules in the solvate can be present in a regular arrangement and/or a non-ordered arrangement. The solvate can comprise either a stoichiometric or nonstoichiometric amount of the solvent molecules. “Solvate” encompasses both solution-phase and isolable solvates. Compounds of the application can form solvates with water (i.e., hydrates) or common organic solvents. Exemplary solvates include, but are not limited to, hydrates, ethanolates, methanolates, and isopropanolates. Methods of solvation are generally known in the art.

Also included within the scope of the application are all isotopes of atoms occurring in the compounds described herein. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds can generally be prepared by conventional techniques known to those skilled in the art or by processes analogous to those described herein, using an appropriate isotopically-labeled reagent in place of the non-labeled reagent otherwise employed.

As used herein, the name of a compound is intended to encompass all possible existing isomeric forms, including stereoisomers (e.g., enantiomers, diastereomers, racemate or racemic mixture, and any mixture thereof) of the compound.

### Compounds

In one general aspect, provided herein are compounds having a pyrazolopyridine core and which are inhibitors of salt inducible kinases (SIKs).

In one embodiment, provided herein is a compound of formula (I):

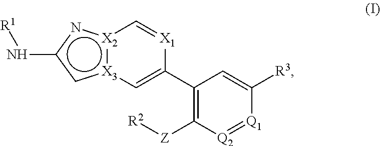

or a stereoisomer, tautomer, pharmaceutically acceptable salt or solvate thereof, wherein:
X₁, X₂, and X₃ are independently N or CH;
Q₁ and Q₂ are independently N or CH;
Z is —O—Cₙ₁H₂ₙ₁—, or —NR⁶—(CH₂)ₙ₂—, provided that O or N is directly bonded to the pyridinyl ring or phenyl ring or pyridazinyl ring;
n1 is 0, 1 or 2, and 2n1 is twice of n1;
n2 is 0 or 1;
R¹ is —C(O)R⁵, phenyl, a 5- to 6-membered heteroaryl having 1-3 nitrogen heteroatoms, or

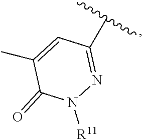

wherein each of the phenyl and 5- to 6-membered heteroaryl is optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino, wherein the 5- to 6-membered heteroaryl is optionally fused to a ring selected from the group consisting of benzene, piperidine, piperazine, and a 5- to 6-membered heteroaryl having 1-2 nitrogen heteroatoms, wherein the 5- to 6-membered heteroaryl is optionally substituted with one or more —CH₃;
R⁵ is cyclopropyl optionally substituted with one or more substituents independently selected from the group consisting of —F, —CH₃, C₁ fluoroalkyl, and phenyl;
R³ is hydrogen, —CH₃, —CH₂CH₃, propynyl, C₁ fluoroalkyl, —CH₂OCH₃, —COOCH₂CH₃, —CN, —Cl, —N(R¹⁰)₂, or —OR¹⁵;
R² is:
   a 4- to 9-membered monocyclic or bicyclic heterocyclyl having 1 to 3 heteroatoms independently selected from oxygen and nitrogen, wherein the 4- to 9-membered monocyclic or bicyclic heterocyclyl is optionally substituted with one or more substituents independently selected from the group consisting of C₁₋₃ alkyl, C₁₋₂ fluoroalkyl, C₁₋₄ hydroxyalkyl, —CD₃, —F, —OH, —OCH₃, oxo, cyclopropyl, —COOR S, —CONH₂, —C(O)CH₃, —N(R¹⁰)₂, —(CH₂)ₘOCH₃, —CN, —CH₂CN, —CH₂(C₅H₄N), —SO₂CH₃, and phenyl,
   a C₃-C₆ cycloalkyl, wherein the C₃-C₆ cycloalkyl is optionally substituted with one or more substituents independently selected from the group consisting of —CH₃, —F, —OH, —OCH₃, —N(R¹²)₂, —CH₂OH, —═O, phenyl, —CN, C₁ fluoroalkyl, —CONH₂, ═NH(OH), —SO₂CH₃, and —CH(CH₃)₂,
   a C₁₋₅ alkyl, wherein the C₁₋₅ alkyl is optionally substituted with one or more substituents independently selected from the group consisting of —OH, —N(R¹⁰)₂, C₁ fluoroalkyl, —COORS, —CON(R⁸)₂, —CH₃, C₃-C₆ cycloalkyl, phenyl, pyridyl, —SO₂CH₃, and —CN, wherein the C₃-C₆ cycloalkyl is optionally substituted with one or more —CH₃,
   a 5- to 6-membered heteroaryl having 1 to 2 nitrogen atoms, wherein the 5- to 6-membered heteroaryl is optionally substituted with one or more substituents independently selected from the group consisting of —CH₃ and C₁ fluoroalkyl,
   or phenyl, or phenyl substituted with —OCHF₂;
R⁶ is hydrogen,
or alternatively, when Z is —NR⁶—(CH₂)ₙ₂— and n2 is 0, R² and R⁶ are taken together with the nitrogen atom to which they are attached to form a 4- to 8-membered monocyclic or bicyclic heterocyclyl having 1-2 nitrogen heteroatoms, wherein the 4- to 8-membered monocyclic or bicyclic heterocyclyl is optionally substituted with one or more substituents independently selected from the group consisting of —OH, C₁ fluoroalkyl, C₁₋₃ hydroxyalkyl, —C(O)N(R¹⁰)₂ and —CH₂N(R¹⁰)²;
each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN;
each R⁹ is independently C₁ fluoroalkyl or —CH₃;
each R¹⁰ is independently hydrogen, —COCH₃, —SO₂CH₃, C₁₋₂ alkyl, or C₁₋₂ fluoroalkyl;
each R¹¹ is independently hydrogen, C₁₋₅ alkyl, —CH₂CH₂—OCHF₂, cyclopropyl, or —CH₂-cyclopropyl; and
each R¹² is independently hydrogen, —CH₃, C₃H₅, —SO₂CH₃, —CH₂CN, —CH₂(C₆H₅), —CH₂COOCH₂CH₃, or a 5- to 6-membered heteroaryl having 1 to 2 nitrogen atoms;
each R¹³ is independently hydrogen or C₁₋₄ alkyl;
each R¹⁴ is independently cyclopropyl or C₁₋₄ alkyl;
each R¹⁵ is independently hydrogen or C₁₋₄ alkyl, wherein the C₁₋₄ alkyl is optionally substituted with one or more substituents independently selected from the group consisting of —OH, —F, and —CN; and
m is 1 or 2.

In some embodiments, provided is a compound of formula (I), or a stereoisomer, tautomer, pharmaceutically acceptable salt or solvate thereof, wherein R¹ is phenyl optionally substituted with one or more substituents independently selected from the group consisting of C₁-C₄ alkyl, —F, C₁ fluoroalkyl, C₁₋₄ hydroxyalkyl, —CON(R⁸)₂, COOR¹³, —OCHF₂, —C(O)R⁹, and SO₂N(R¹⁴)₂; each R⁸ is independently hydrogen, cyclopropyl, or C₁₋₄ alkyl, wherein the C₁₋₄ alkyl is optionally substituted with one or more —F; each R⁹ is independently C₁ fluoroalkyl; each R¹³ is independently hydrogen or C₁₋₄ alkyl; and each R¹⁴ is independently cyclopropyl or C₁₋₄ alkyl.

In some embodiments, provided is a compound of formula (I), or a stereoisomer, tautomer, pharmaceutically acceptable salt or solvate thereof, wherein R¹ is phenyl optionally substituted with —COOCH₃, —C(O)N(H)CH₂CH₃, —C(O)N(H)CH₂CF₃, —OCH₃, —OCHF₂,

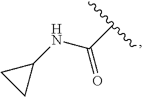 [SMILES: *C(=O)NC1CC1]

SO₂N(CH₃)₂, and

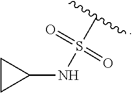 [SMILES: *S(=O)(=O)NC1CC1]

In some embodiments, provided is a compound of formula (I), or a stereoisomer, tautomer, pharmaceutically acceptable salt or solvate thereof, wherein R¹ is —C(O)R⁵, and R⁵ is cyclopropyl optionally substituted with one or more substituents independently selected from the group consisting of —F, —CH₃, C₁ fluoroalkyl, and phenyl.

In some embodiments, provided is a compound of formula (I), or a stereoisomer, tautomer, pharmaceutically acceptable salt or solvate thereof, wherein R¹ is —C(O)R⁵, and R⁵ is cyclopropyl.

In some embodiments, provided is a compound of formula (I), or a stereoisomer, tautomer, pharmaceutically acceptable salt or solvate thereof, wherein R¹ is —C(O)R⁵, and R⁵ is cyclopropyl substituted with 1-2 fluoro atoms.

In some embodiments, provided is a compound of formula (I), or a stereoisomer, tautomer, pharmaceutically acceptable salt or solvate thereof, wherein R¹ is —C(O)R⁵, and R⁵ is cyclopropyl substituted with —CH₃.

In some embodiments, provided is a compound of formula (I), or a stereoisomer, tautomer, pharmaceutically acceptable salt or solvate thereof, wherein R¹ is —C(O)R⁵, and R⁵ is cyclopropyl substituted with —CF₃.

In some embodiments, provided is a compound of formula (I), or a stereoisomer, tautomer, pharmaceutically acceptable salt or solvate thereof, wherein R¹ is —C(O)R⁵, and R⁵ is cyclopropyl substituted with phenyl.

In some embodiments, provided is a compound of formula (I), or a stereoisomer, tautomer, pharmaceutically acceptable salt or solvate thereof, wherein R¹ is a 5- to 6-membered heteroaryl having 1-3 nitrogen heteroatoms optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino, wherein the 5- to 6-membered heteroaryl is optionally fused to a ring selected from the group consisting of benzene, piperidine, piperazine, and a 5- to 6-membered heteroaryl having 1-2 nitrogen heteroatoms, wherein the 5- to 6-membered heteroaryl is optionally substituted with one or more —CH₃, each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R⁹ is independently C₁ fluoroalkyl or —CH₃, each R¹⁰ is independently hydrogen, —COCH₃, —SO₂CH₃, C₁₋₂ alkyl, or C₁₋₂ fluoroalkyl; each R¹³ is independently hydrogen or C₁₋₄ alkyl; and each R¹⁴ is independently cyclopropyl or C₁₋₄ alkyl.

In some embodiments, provided is a compound of formula (I), or a stereoisomer, tautomer, pharmaceutically acceptable salt or solvate thereof, wherein R¹ is a 5- to 6-membered heteroaryl having 1-3 nitrogen heteroatoms optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino; each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN, wherein the C₁₋₄ alkyl is optionally substituted with one or more —F; each R⁹ is independently C₁ fluoroalkyl or —CH₃, each R¹⁰ is independently hydrogen, —COCH₃, —SO₂CH₃, C₁₋₂ alkyl, or C₁₋₂ fluoroalkyl; each R¹³ is independently hydrogen or C₁₋₄ alkyl; and each R¹⁴ is independently cyclopropyl or C₁₋₄ alkyl.

In some embodiments, provided is a compound of formula (I), or a stereoisomer, tautomer, pharmaceutically acceptable salt or solvate thereof, wherein R¹ is

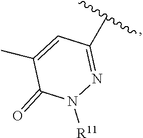

wherein R¹¹ is hydrogen, —CH₃, —CH₂CH₃, —CH₂CH₂—OCHF₂, cyclopropyl, or —CH₂-cyclopropyl.

In some embodiments, provided is a compound of formula (I), or a stereoisomer, tautomer, pharmaceutically acceptable salt or solvate thereof, wherein R¹ is a 5- to 6-membered heteroaryl having 1-3 nitrogen heteroatoms optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino, wherein the 5- to 6-membered heteroaryl is fused to a ring selected from the group consisting of benzene, piperidine, piperazine, and a 5- to 6-membered heteroaryl having 1-2 nitrogen heteroatoms, wherein the 5- to 6-membered heteroaryl is optionally substituted with one or more —CH₃, each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R⁹ is independently C₁ fluoroalkyl or —CH₃, each R¹⁰ is independently hydrogen, —COCH₃, —SO₂CH₃, C₁₋₂ alkyl, or C₁₋₂ fluoroalkyl; each R¹³ is independently hydrogen or C₁₋₄ alkyl; and each R¹⁴ is independently cyclopropyl or C₁₋₄ alkyl.

In some embodiments, provided is a compound of formula (I), or a stereoisomer, tautomer, pharmaceutically acceptable salt or solvate thereof, wherein R¹ is indazolyl, 4,5,6,7-tetrahydropyrazolo[1,5-a]pyrazinyl, 5,6,7,8-tetrahydro-2,7-naphthyridinyl, 5,6,7,8-tetrahydro-2,6-naphthyridinyl, pyrrolo[2,3-b]pyridinyl or imidazo[1,2-b]pyridazinyl.

In some embodiments, provided is a compound of formula (I), or a stereoisomer, tautomer, pharmaceutically acceptable salt or solvate thereof, wherein R¹ is triazolyl optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino, each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R⁹ is independently C₁ fluoroalkyl or —CH₃, each R¹⁰ is independently hydrogen, —COCH₃, —SO₂CH₃, C₁₋₂ alkyl, or C₁₋₂ fluoroalkyl; each R¹³ is independently hydrogen or C₁₋₄ alkyl; and each R¹⁴ is independently cyclopropyl or C₁₋₄ alkyl.

In some embodiments, provided is a compound of formula (I), or a stereoisomer, tautomer, pharmaceutically acceptable salt or solvate thereof, wherein R¹ is triazolyl optionally substituted with one or more substituents independently selected from the group consisting of —C(O)NHCH₃, —F, —CH₃, —CH₂CH₃, —NHCH₃, —CHF₂, —C(O)CF₃, —CH₂C(OH)(CH₃)₂, —CH(CH₃)₂, C(CH₃)₃, —C₃H₅, —C(O)N(CH₃)₂, —CH₂OCH₃, —OCHF₂, —CH(OH)CHCF₃, —CO₂H, —CO₂CH₃, —SO₂CH₃, —SO₂N(CH₃)₂,

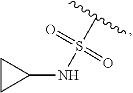 [SMILES: *S(=O)(=O)NC1CC1]

cyclopropyl, pyrrolidinyl, morpholino, and morpholinomethyl.

In some embodiments, provided is a compound of formula (I), or a stereoisomer, tautomer, pharmaceutically acceptable salt or solvate thereof, wherein R¹ is pyrazolyl optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino, each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R⁹ is independently C₁ fluoroalkyl or —CH₃, each R¹⁰ is independently hydrogen, —COCH₃, —SO₂CH₃, C₁₋₂ alkyl, or C₁₋₂ fluoroalkyl; each R¹³ is independently hydrogen or C₁₋₄ alkyl; and each R¹⁴ is independently cyclopropyl or C₁₋₄ alkyl.

In some embodiments, provided is a compound of formula (I), or a stereoisomer, tautomer, pharmaceutically acceptable salt or solvate thereof, wherein R¹ is pyrazolyl optionally substituted with one or more substituents independently selected from the group consisting of —C(O)NHCH₃, —F, —CH₃, —CH₂CH₃, —NHCH₃, —CHF₂, —C(O)CF₃, —CH₂C(OH)(CH₃)₂, —CH(CH₃)₂, C(CH₃)₃, —C₃H₅, —C(O)N(CH₃)₂, —CH₂OCH₃, —OCHF₂, —CH(OH)CHCF₃, —CO₂H, —CO₂CH₃, —SO₂CH₃, —SO₂N(CH₃)₂,

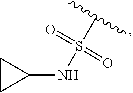 [SMILES: *S(=O)(=O)NC1CC1]

cyclopropyl, pyrrolidinyl, morpholino, and morpholinomethyl.

In some embodiments, provided is a compound of formula (I), or a stereoisomer, tautomer, pharmaceutically acceptable salt or solvate thereof, wherein R¹ is pyridinyl optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino, each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R⁹ is independently C₁ fluoroalkyl or —CH₃, each R¹⁰ is independently hydrogen, —COCH₃, —SO₂CH₃, C₁₋₂ alkyl, or C₁₋₂ fluoroalkyl; each R¹³ is independently hydrogen or C₁₋₄ alkyl; and each R¹⁴ is independently cyclopropyl or C₁₋₄ alkyl.

In some embodiments, provided is a compound of formula (I), or a stereoisomer, tautomer, pharmaceutically acceptable salt or solvate thereof, wherein R¹ is pyridinyl optionally substituted with one or more substituents independently selected from the group consisting of —C(O)NHCH₃, —F, —CH₃, —CH₂CH₃, —NHCH₃, —CHF₂, —C(O)CF₃, —CH₂C(OH)(CH₃)₂, —CH(CH₃)₂, C(CH₃)₃, —C₃H₅, —C(O)N(CH₃)₂, —CH₂OCH₃, —OCHF₂, —CH(OH)CHCF₃, —CO₂H, —CO₂CH₃, —SO₂CH₃, —SO₂N(CH₃)₂,

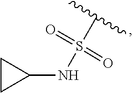 [SMILES: *S(=O)(=O)NC1CC1]

cyclopropyl, pyrrolidinyl, morpholino, and morpholinomethyl.

In some embodiments, provided is a compound of formula (I), or a stereoisomer, tautomer, pharmaceutically acceptable salt or solvate thereof, wherein R¹ is pyrazinyl optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino, each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R⁹ is independently C₁ fluoroalkyl or —CH₃, each R¹⁰ is independently hydrogen, —COCH₃, —SO₂CH₃, C₁₋₂ alkyl, or C₁₋₂ fluoroalkyl; each R¹³ is independently hydrogen or C₁₋₄ alkyl; and each R¹⁴ is independently cyclopropyl or C₁₋₄ alkyl.

In some embodiments, provided is a compound of formula (I), or a stereoisomer, tautomer, pharmaceutically acceptable salt or solvate thereof, wherein R¹ is pyrazinyl optionally substituted with one or more substituents independently selected from the group consisting of —C(O)NHCH₃, —F, —CH₃, —CH₂CH₃, —NHCH₃, —CHF₂, —C(O)CF₃, —CH₂C(OH)(CH₃)₂, —CH(CH₃)₂, C(CH₃)₃, —C₃H₅, —C(O)N(CH₃)₂, —CH₂OCH₃, —OCHF₂, —CH(OH)CHCF₃, —CO₂H, —CO₂CH₃, —SO₂CH₃, —SO₂N(CH₃)₂,

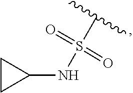 [SMILES: *S(=O)(=O)NC1CC1]

cyclopropyl, pyrrolidinyl, morpholino, and morpholinomethyl.

In some embodiments, provided is a compound of formula (I), or a stereoisomer, tautomer, pharmaceutically acceptable salt or solvate thereof, wherein R¹ is pyrimidinyl optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino, each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R⁹ is independently C₁ fluoroalkyl or —CH₃, each R¹⁰ is independently hydrogen, —COCH₃, —SO₂CH₃, C₁₋₂ alkyl, or C₁₋₂ fluoroalkyl; each R¹³ is independently hydrogen or C₁₋₄ alkyl; and each R¹⁴ is independently cyclopropyl or C₁₋₄ alkyl.

In some embodiments, provided is a compound of formula (I), or a stereoisomer, tautomer, pharmaceutically acceptable salt or solvate thereof, wherein R¹ is pyrimidinyl optionally substituted with one or more substituents independently selected from the group consisting of —C(O)NHCH₃, —F, —CH₃, —CH₂CH₃, —NHCH₃, —CHF₂, —C(O)CF₃, —CH₂C(OH)(CH₃)₂, —CH(CH₃)₂, C(CH₃)₃, —C₃H₅, —C(O)N(CH₃)₂, —CH₂OCH₃, —OCHF₂, —CH(OH)CHCF₃, —CO₂H, —CO₂CH₃, —SO₂CH₃, —SO₂N(CH₃)₂,

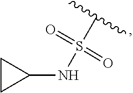 [SMILES: *S(=O)(=O)NC1CC1]

cyclopropyl, pyrrolidinyl, morpholino, and morpholinomethyl.

In some embodiments, provided is a compound of formula (I), or a stereoisomer, tautomer, pharmaceutically acceptable salt or solvate thereof, wherein R¹ is pyridazinyl optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino, each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R⁹ is independently C₁ fluoroalkyl or —CH₃, each R¹⁰ is independently hydrogen, —COCH₃, —SO₂CH₃, C₁₋₂ alkyl, or C₁₋₂ fluoroalkyl; each R¹³ is independently hydrogen or C₁₋₄ alkyl; and each R¹⁴ is independently cyclopropyl or C₁₋₄ alkyl.

In some embodiments, provided is a compound of formula (I), or a stereoisomer, tautomer, pharmaceutically acceptable salt or solvate thereof, wherein R¹ is pyridazinyl optionally substituted with one or more substituents independently selected from the group consisting of —C(O)NHCH₃, —F, —CH₃, —CH₂CH₃, —NHCH₃, —CHF₂, —C(O)CF₃, —CH₂C(OH)(CH₃)₂, —CH(CH₃)₂, C(CH₃)₃, —C₃H₅, —C(O)N(CH₃)₂, —CH₂OCH₃, —OCHF₂, —CH(OH)CHCF₃, —CO₂H, —CO₂CH₃, —SO₂CH₃, —SO₂N(CH₃)₂,

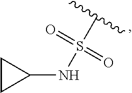 [SMILES: *S(=O)(=O)NC1CC1]

cyclopropyl, pyrrolidinyl, morpholino, and morpholinomethyl.

In some embodiments, provided is a compound of formula (I), or a stereoisomer, tautomer, pharmaceutically acceptable salt or solvate thereof, wherein R¹ is:

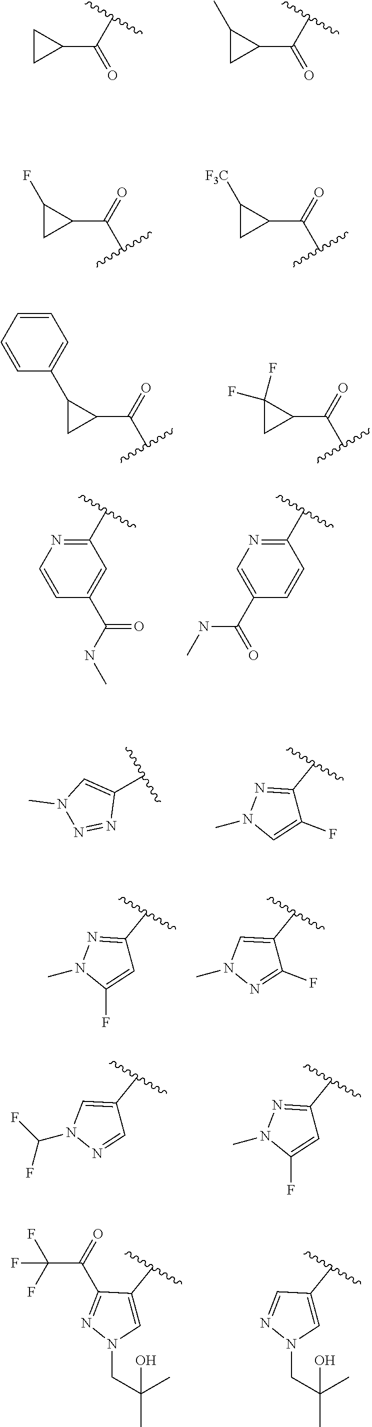

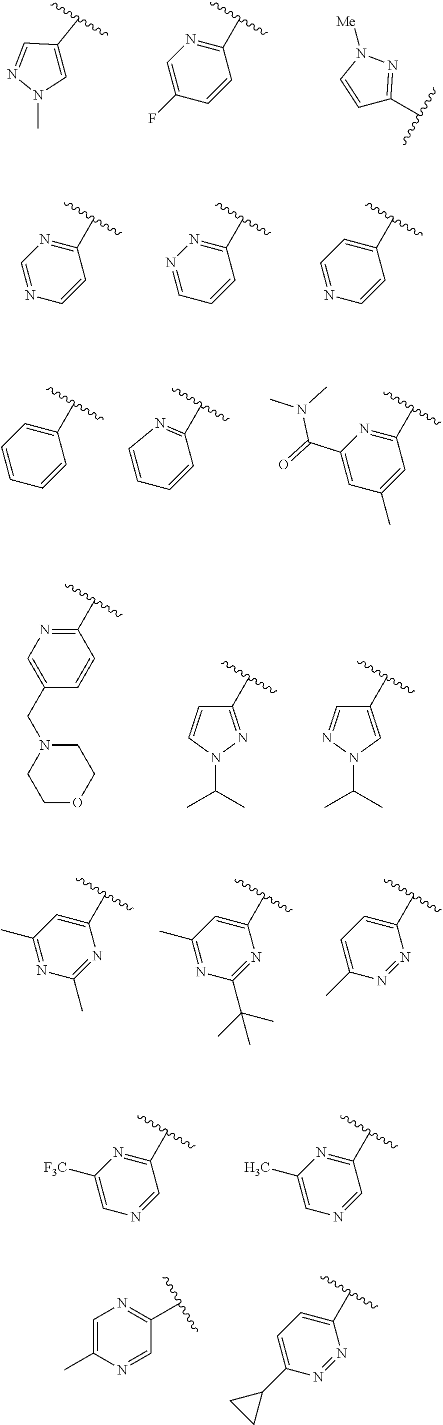

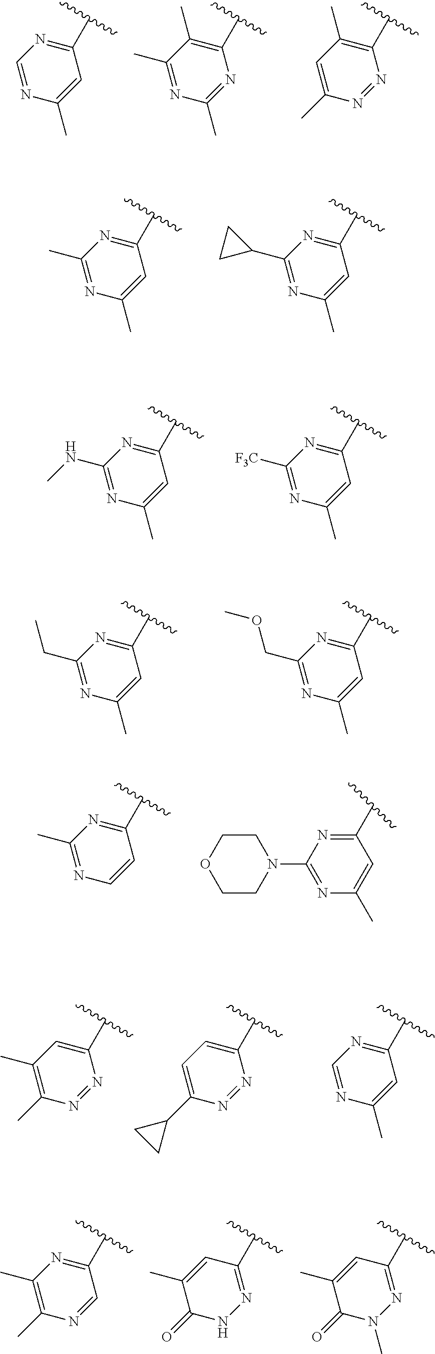

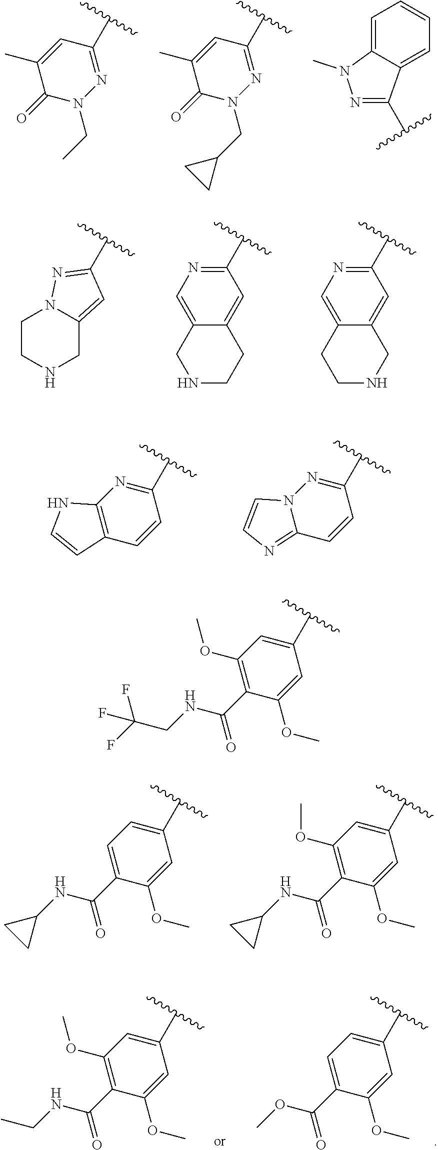

In some embodiments, provided is a compound of formula (I), or a stereoisomer, tautomer, pharmaceutically acceptable salt or solvate thereof, wherein R³ is hydrogen.

In some embodiments, provided is a compound of formula (I), or a stereoisomer, tautomer, pharmaceutically acceptable salt or solvate thereof, wherein R³ is —CH₃ or CH₂CH₃ or propynyl.

In some embodiments, provided is a compound of formula (I), or a stereoisomer, tautomer, pharmaceutically acceptable salt or solvate thereof, wherein R³ is C₁ fluoroalkyl, such as CH₂F, CHF₂, or CF₃.

In some embodiments, provided is a compound of formula (I), or a stereoisomer, tautomer, pharmaceutically acceptable salt or solvate thereof, wherein R³ is —CN.

In some embodiments, provided is a compound of formula (I), or a stereoisomer, tautomer, pharmaceutically acceptable salt or solvate thereof, wherein R³ is —Cl.

In some embodiments, provided is a compound of formula (I), or a stereoisomer, tautomer, pharmaceutically acceptable salt or solvate thereof, wherein R³ is —N(R¹⁰)₂, wherein each R¹⁰ is independently hydrogen, C₁₋₂ alkyl, or C₁₋₂ fluoroalkyl.

In some embodiments, provided is a compound of formula (I), or a stereoisomer, tautomer, pharmaceutically acceptable salt or solvate thereof, wherein R³ is —OR¹⁵, wherein each R¹⁵ is independently hydrogen or C₁₋₄ alkyl, wherein the C₁₋₄ alkyl is optionally substituted with one or more substituents independently selected from the group consisting of —OH, —F, and —CN.

In some embodiments, provided is a compound of formula (I), or a stereoisomer, tautomer, pharmaceutically acceptable salt or solvate thereof, wherein R² is a 4- to 9-membered monocyclic or bicyclic heterocyclyl having 1 to 3 heteroatoms independently selected from oxygen and nitrogen, wherein the 4- to 9-membered monocyclic or bicyclic heterocyclyl is optionally substituted with one or more substituents independently selected from the group consisting of C₁₋₃ alkyl, C₁₋₂ fluoroalkyl, C₁₋₄ hydroxyalkyl, —CD₃, —F, —OH, —OCH₃, oxo, cyclopropyl, —COOR⁸, —CONH₂, —C(O)CH₃, —N(R¹⁰)₂, —(CH₂)ₘOCH₃, —CN, —CH₂CN, —CH₂(C₅H₄N), —SO₂CH₃, phenyl,

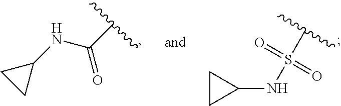

wherein each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R¹⁰ is independently hydrogen, —COCH₃, —SO₂CH₃, C₁₋₂ alkyl, or C₁₋₂ fluoroalkyl; and m is 1 or 2.

In some embodiments, provided is a compound of formula (I), or a stereoisomer, tautomer, pharmaceutically acceptable salt or solvate thereof, wherein R² is a 4- to 9-membered monocyclic or bicyclic heterocyclyl having 1 to 3 heteroatoms independently selected from oxygen and nitrogen, wherein the 4- to 9-membered monocyclic or bicyclic heterocyclyl is optionally substituted with one or more substituents independently selected from the group consisting of —CH₃, —CD₃, —OCH₃, —CH₂CH₃, —CH(CH₃)₂, —CH₂CF₃, —CF₃, —CH₂OH, —CH₂C(OH)(CH₃)₂, —F, —OH, —NH₂, —CN, oxo, cyclopropyl, —COOC(CH₃)₃, —CONH₂, —C(O)CH₃, —CH₂CN, —CH₂CH₂OCH₃, —CH₂(C₅H₄N), —SO₂CH₃, phenyl,

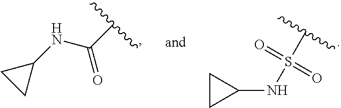 [SMILES: *C(=O)NC1CC1 ; *S(=O)(=O)NC1CC1]

In some embodiments, provided is a compound of formula (I), or a stereoisomer, tautomer, pharmaceutically acceptable salt or solvate thereof, wherein R² is a 4- to 7-membered monocyclic heterocyclyl having 1 to 3 heteroatoms independently selected from oxygen and nitrogen, wherein the 4- to 7-membered monocyclic heterocyclyl is optionally substituted with one or more substituents independently selected from the group consisting of C₁₋₃ alkyl, C₁₋₂ fluoroalkyl, C₁₋₄ hydroxyalkyl, —CD₃, —F, —OH, —OCH₃, oxo, cyclopropyl, —COOR⁸, —CONH₂, —C(O)CH₃, —N(R¹⁰)₂, —(CH₂)ₘOCH₃, —CN, —CH₂CN, —CH₂(C₅H₄N), —SO₂CH₃, and phenyl; wherein each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R¹⁰ is independently hydrogen, —COCH₃, —SO₂CH₃, C₁₋₂ alkyl, or C₁₋₂ fluoroalkyl; and m is 1 or 2.

In some embodiments, provided is a compound of formula (I), or a stereoisomer, tautomer, pharmaceutically acceptable salt or solvate thereof, wherein R² is a 4- to 7-membered monocyclic heterocyclyl having 1 to 3 heteroatoms independently selected from oxygen and nitrogen, wherein the 4- to 7-membered monocyclic heterocyclyl is optionally substituted with one or more substituents independently selected from the group consisting of —CH₃, —CD₃, —OCH₃, —CH₂CH₃, —CH(CH₃)₂, —CH₂CF₃, —CF₃, —CH₂OH, —CH₂C(OH)(CH₃)₂, —F, —OH, —NH₂, —CN, oxo, cyclopropyl, —COOC(CH₃)₃, —CONH₂, —C(O)CH₃, —CH₂CN, —CH₂CH₂OCH₃, —CH₂(C₅H₄N), —SO₂CH₃, phenyl,

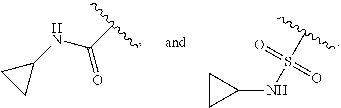 [SMILES: *C(=O)NC1CC1 ; *S(=O)(=O)NC1CC1]

In some embodiments, provided is a compound of formula (I), or a stereoisomer, tautomer, pharmaceutically acceptable salt or solvate thereof, wherein R² is a 4- to 7-membered monocyclic heterocyclyl selected from the group consisting of pyrrolidinyl, piperidinyl, oxazepanyl, morpholinyl, azetidinyl, and oxetanyl, wherein each of the pyrrolidinyl, piperidinyl, oxazepanyl, morpholinyl, azetidinyl, oxetanyl, and 1,1-dioxo-1,2,5-thiadiazolidinyl, is optionally substituted with one or more substituents independently selected from the group consisting of C₁₋₃ alkyl, C₁₋₂ fluoroalkyl, C₁₋₄ hydroxyalkyl, —CD₃, —F, —OH, —OCH₃, oxo, cyclopropyl, —COOR⁸, —CONH₂, —C(O)CH₃, —N(R¹⁰)₂, —(CH₂)ₘOCH₃, —CN, —CH₂CN, —CH₂(C₅H₄N), —SO₂CH₃, and phenyl; wherein each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R¹⁰ is independently hydrogen, —COCH₃, —SO₂CH₃, C₁₋₂ alkyl, or C₁₋₂ fluoroalkyl; and m is 1 or 2.

In some embodiments, provided is a compound of formula (I), or a stereoisomer, tautomer, pharmaceutically acceptable salt or solvate thereof, wherein R² is a 4- to 7-membered monocyclic heterocyclyl selected from the group consisting of pyrrolidinyl, piperidinyl, oxazepanyl, morpholinyl, azetidinyl, and oxetanyl, wherein each of the pyrrolidinyl, piperidinyl, oxazepanyl, morpholinyl, azetidinyl, oxetanyl, and 1,1-dioxo-1,2,5-thiadiazolidinyl, is optionally substituted with one or more substituents independently selected from the group consisting of —CH₃, —CD₃, —OCH₃, —CH₂CH₃, —CH(CH₃)₂, —CH₂CF₃, —CF₃, —CH₂OH, —CH₂C(OH)(CH₃)₂, —F, —OH, —NH₂, —CN, oxo, cyclopropyl, —COOC(CH₃)₃, —CONH₂, —C(O)CH₃, —CH₂CN, —CH₂CH₂OCH₃, —CH₂(C₅H₄N), —SO₂CH₃, phenyl,

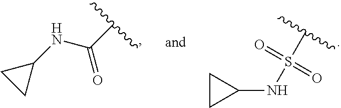 [SMILES: *C(=O)NC1CC1 ; *S(=O)(=O)NC1CC1]

In some embodiments, provided is a compound of formula (I), or a stereoisomer, tautomer, pharmaceutically acceptable salt or solvate thereof, wherein R² is a 4- to 7-membered monocyclic heterocyclyl selected from the group consisting of:

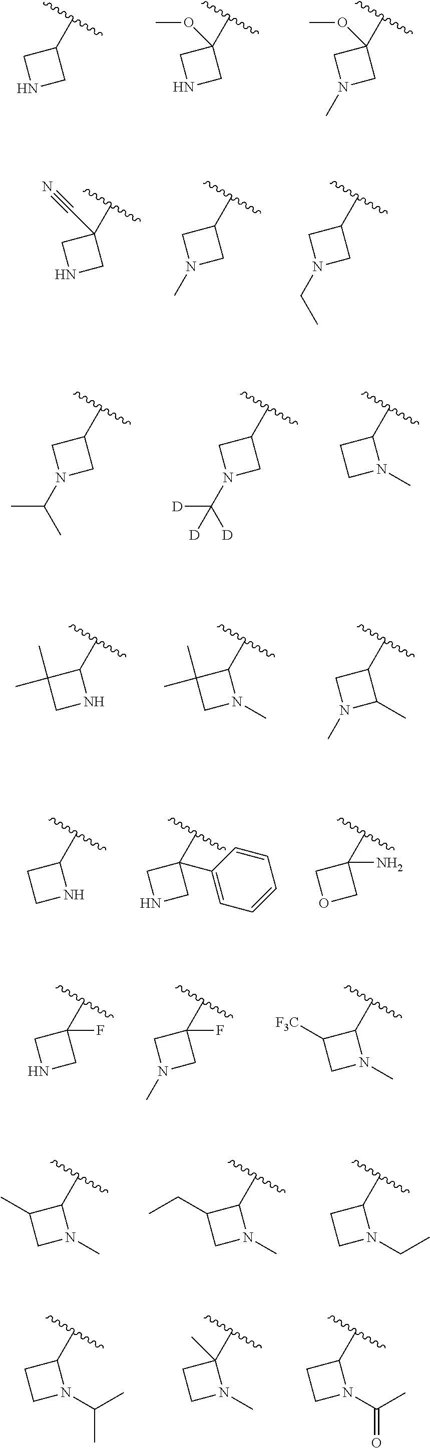

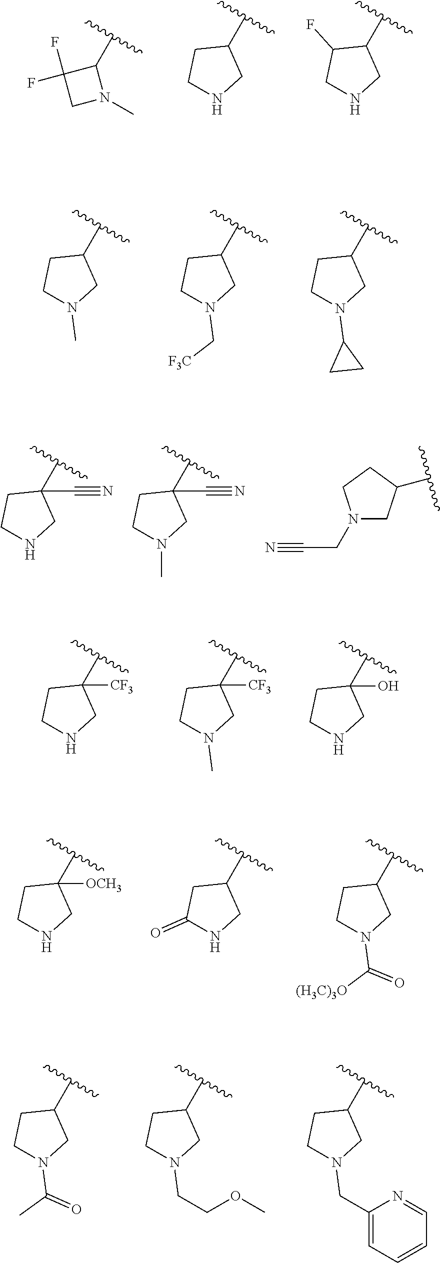

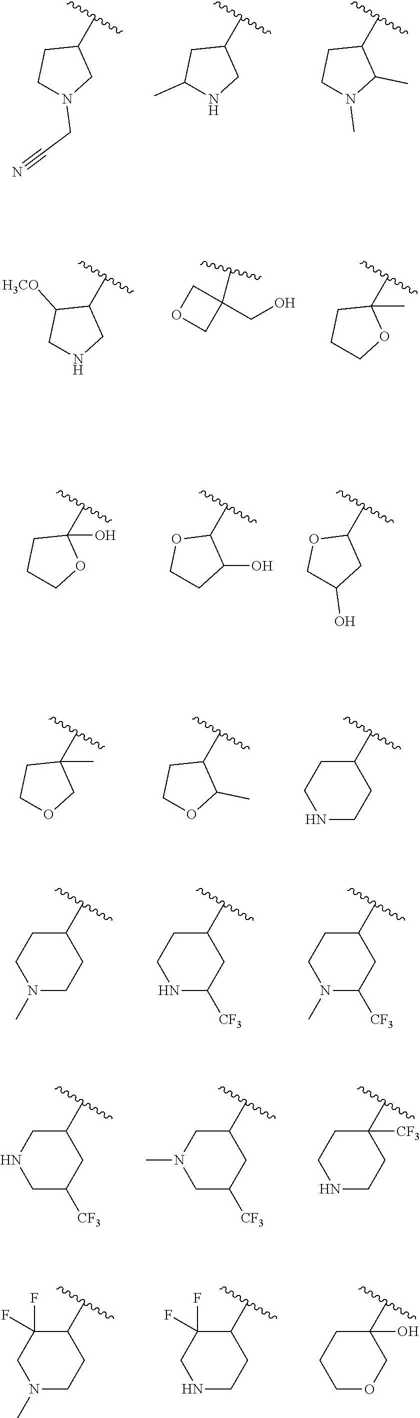

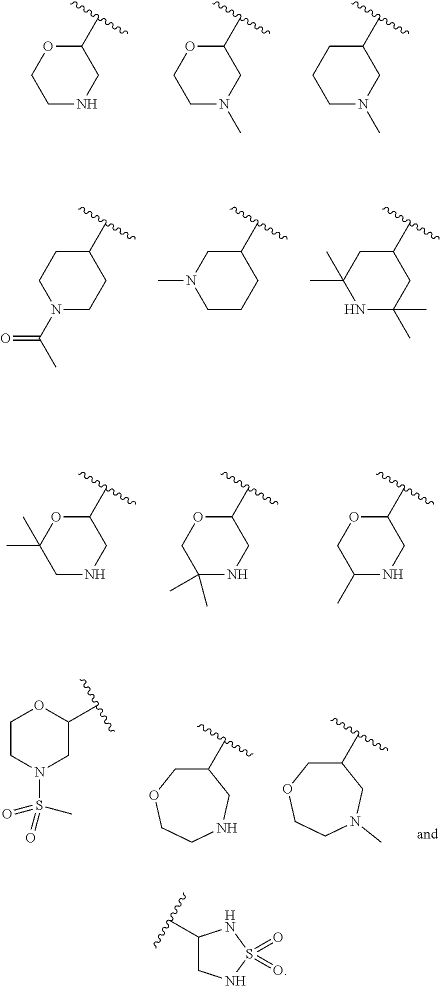

In some embodiments, provided is a compound of formula (I), or a stereoisomer, tautomer, pharmaceutically acceptable salt or solvate thereof, wherein R² is a 6- to 9-membered bicyclic heterocyclyl having 1 to 2 heteroatoms independently selected from oxygen and nitrogen, wherein the 6- to 9-membered bicyclic heterocyclyl is optionally substituted with one or more substituents independently selected from the group consisting of C₁₋₃ alkyl, C₁₋₂ fluoroalkyl, C₁₋₄ hydroxyalkyl, —CD₃, —F, —OH, —OCH₃, oxo, cyclopropyl, —COOR⁸, —CONH₂, —C(O)CH₃, —N(R¹⁰)₂, —(CH₂)ₘOCH₃, —CN, —CH₂CN, —CH₂(C₅H₄N), —SO₂CH₃, phenyl,

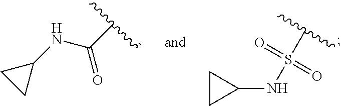

wherein each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R¹⁰ is independently hydrogen, —COCH₃, —SO₂CH₃, C₁₋₂ alkyl, or C₁₋₂ fluoroalkyl; and m is 1 or 2.

In some embodiments, provided is a compound of formula (I), or a stereoisomer, tautomer, pharmaceutically acceptable salt or solvate thereof, wherein R² is a 6- to 9-membered bicyclic heterocyclyl having 1 to 2 heteroatoms independently selected from oxygen and nitrogen, wherein the 6- to 9-membered bicyclic heterocyclyl is optionally substituted with one or more substituents independently selected from the group consisting of —CH₃, —CD₃, —OCH₃, —CH₂CH₃, —CH(CH₃)₂, —CH₂CF₃, —CF₃, —CH₂OH, —CH₂C(OH)(CH₃)₂, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), —SO₂CH₃, phenyl,

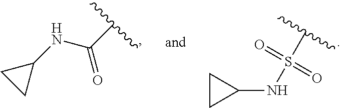 [SMILES: *C(=O)NC1CC1 ; *S(=O)(=O)NC1CC1]

In some embodiments, provided is a compound of formula (I), or a stereoisomer, tautomer, pharmaceutically acceptable salt or solvate thereof, wherein R² is a 6- to 9-membered bicyclic heterocyclyl selected from the group consisting of 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonanyl, 6-oxaspiro[3.4]octanyl, and 1-oxaspiro[4.4]nonanyl wherein each of the 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonanyl, 6-oxaspiro[3.4]octanyl, and 1-oxaspiro[4.4]nonanyl is optionally substituted with one or more substituents independently selected from the group consisting of C₁₋₃ alkyl, C₁₋₂ fluoroalkyl, C₁₋₄ hydroxyalkyl, —CD₃, —F, —OH, —OCH₃, oxo, cyclopropyl, —COOR S, —CONH₂, —C(O)CH₃, —N(R¹⁰)₂, —(CH₂)ₘOCH₃, —CN, —CH₂CN, —CH₂(C₅H₄N), —SO₂CH₃, phenyl,

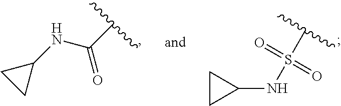

wherein each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN; each R¹⁰ is independently hydrogen, —COCH₃, —SO₂CH₃, C₁₋₂ alkyl, or C₁₋₂ fluoroalkyl; and m is 1 or 2.

In some embodiments, provided is a compound of formula (I), or a stereoisomer, tautomer, pharmaceutically acceptable salt or solvate thereof, wherein R² is a 6- to 9-membered bicyclic heterocyclyl selected from the group consisting of 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonanyl, 6-oxaspiro[3.4]octanyl, and 1-oxaspiro[4.4]nonanyl wherein each of the 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonanyl, 6-oxaspiro[3.4]octanyl, and 1-oxaspiro[4.4]nonanyl is optionally substituted with one or more substituents independently selected from the group consisting of —CH₃, —CD₃, —OCH₃, —CH₂CH₃, —CH(CH₃)₂, —CH₂CF₃, —CF₃, —CH₂OH, —CH₂C(OH)(CH₃)₂, —F, —OH, —NH₂, —CN, oxo, cyclopropyl, —COOC(CH₃)₃, —CONH₂, —C(O)CH₃, —CH₂CN, —CH₂CH₂OCH₃, —CH₂(C₅H₄N), —SO₂CH₃, phenyl,

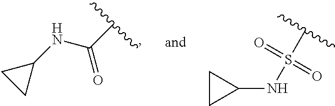 [SMILES: *C(=O)NC1CC1 ; *S(=O)(=O)NC1CC1]

In some embodiments, provided is a compound of formula (I), or a stereoisomer, tautomer, pharmaceutically acceptable salt or solvate thereof, wherein R² is a 6- to 9-membered bicyclic heterocyclyl selected from the group consisting of 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonanyl, 6-oxaspiro[3.4]octanyl, and 1-oxaspiro[4.4]nonanyl, wherein each of the 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonanyl, 6-oxaspiro[3.4]octanyl, and 1-oxaspiro[4.4]nonanyl is optionally substituted with one or more substituents independently selected from the group consisting of —CH₃, —CH₂CH₃, —CH(CH₃)₂, —CH₂C(OH)(CH₃)₂, —COOC(CH₃)₃, —CN, —SO₂CH₃, oxo,

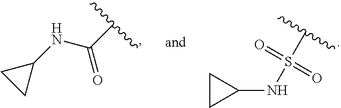 [SMILES: *C(=O)NC1CC1 ; *S(=O)(=O)NC1CC1]

In some embodiments, provided is a compound of formula (I), or a stereoisomer, tautomer, pharmaceutically acceptable salt or solvate thereof, wherein R² is a 6- to 9-membered bicyclic heterocyclyl selected from the group consisting of:

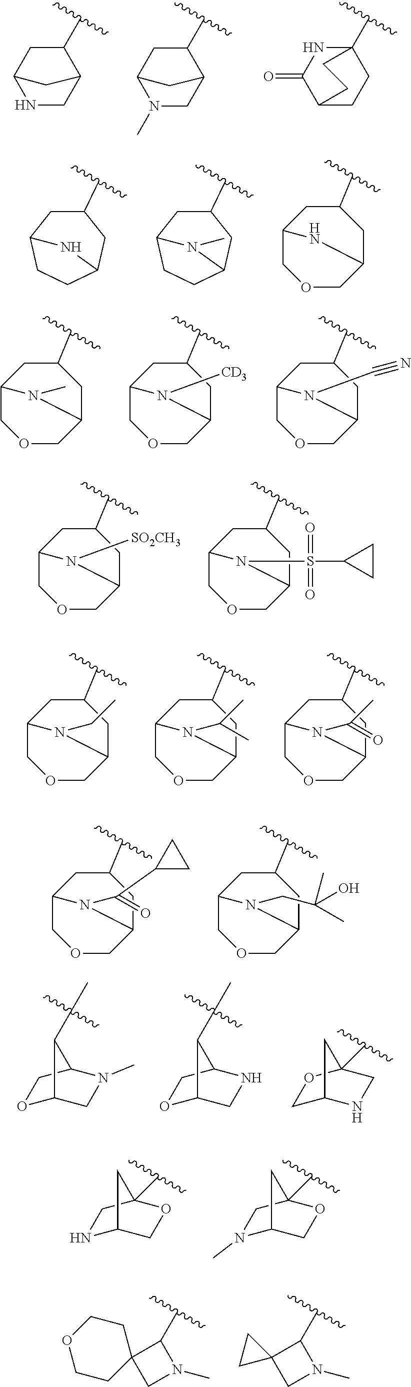

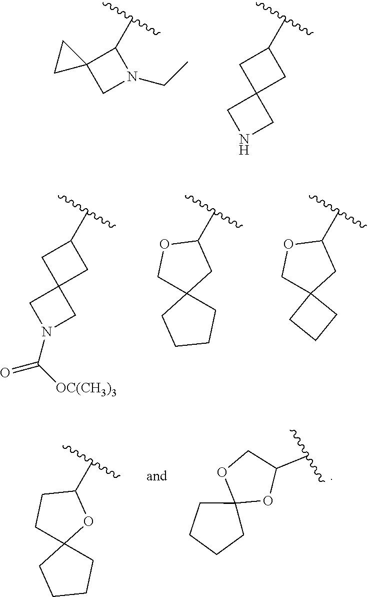

In some embodiments, provided is a compound of formula (I), or a stereoisomer, tautomer, pharmaceutically acceptable salt or solvate thereof, wherein R² is a C₃-C₆ cycloalkyl, wherein the C₃-C₆ cycloalkyl is optionally substituted with one or more substituents independently selected from the group consisting of —CH₃, —F, —OH, —OCH₃, —N(R¹²)₂, —CH₂OH, ═O, phenyl, —CN, C₁ fluoroalkyl, —CONH₂, ═NH(OH), —SO₂CH₃, and —CH(CH₃)₂, wherein each R¹² is independently hydrogen, —CH₃, C₃H₅, —SO₂CH₃, —CH₂CN, —CH₂(C₆H₅), —CH₂COOCH₂CH₃, or a 5- to 6-membered heteroaryl having 1 to 2 nitrogen atoms, wherein the 5- to 6-membered heteroaryl is optionally substituted with one or more —CH₃.

In some embodiments, provided is a compound of formula (I), or a stereoisomer, tautomer, pharmaceutically acceptable salt or solvate thereof, wherein R² is a cyclopropyl, cyclobutyl, cyclopentyl or cyclohexyl, wherein each of the cyclopropyl, cyclobutyl, cyclopentyl and cyclohexyl is optionally substituted with one or more substituents independently selected from the group consisting of —NH₂, —OH, —OCH₃, —CH₂OH, ═O, —CONH₂, phenyl, —CH₃, —CF₃, —F, —NHCH₃, —NH(C₃H₅), —NHCH₂CN, —NH—COOCH₂CH₃, —NH(C₅H₄N), —NH(C₆H₅),

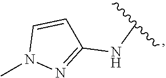 [SMILES: *Nc1ccn(C)n1]

═NH(OH), —SO₂CH₃, and —CH(CH₃)₂.

In some embodiments, provided is a compound of formula (I), or a stereoisomer, tautomer, pharmaceutically acceptable salt or solvate thereof, wherein R² is a C₃-C₆ cycloalkyl selected from the group consisting of:

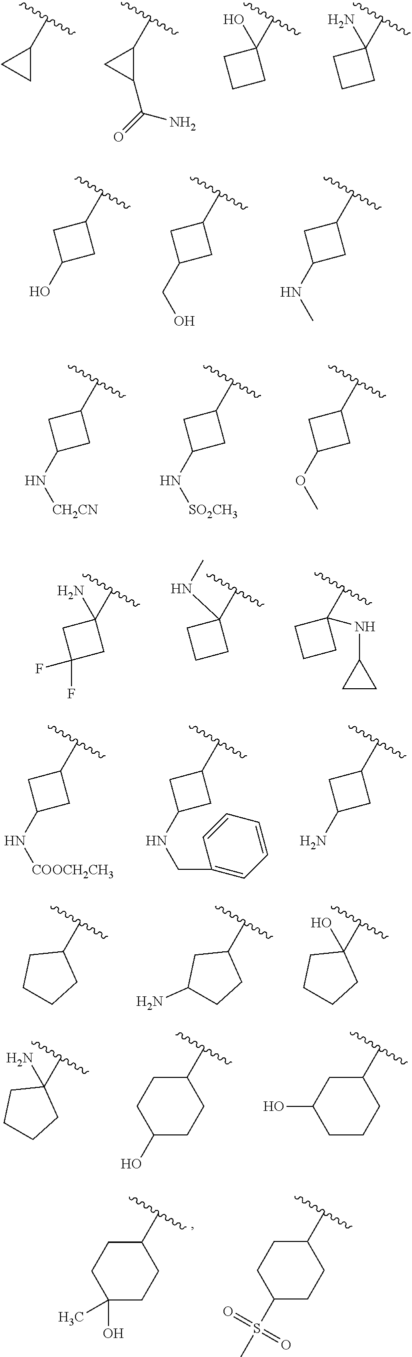

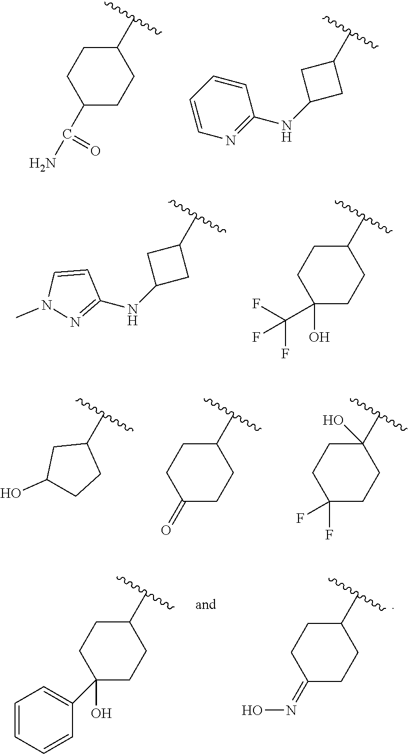

In some embodiments, provided is a compound of formula (I), or a stereoisomer, tautomer, pharmaceutically acceptable salt or solvate thereof, wherein R² is a C₁₋₅ alkyl optionally substituted with one or more substituents independently selected from the group consisting of —N(CH₃)₂, —OH, —CF₃, —NH₂, —COOC(CH₃)₃, —NH—COCH₃, —NH—SO₂CH₃, —COONH₂, —COON(CH₃)₂, cyclopropyl, methylcyclopropyl, —CH₃, C₃-C₆ cycloalkyl, —SO₂CH₃, phenyl, pyridyl, and —CN.

In some embodiments, provided is a compound of formula (I), or a stereoisomer, tautomer, pharmaceutically acceptable salt or solvate thereof, wherein R² is selected from the group consisting of —CH₂CH₂N(CH₃)₂, —C(OH)(CH₃)CF₃, —C(CH₃)₂OH, —CH(OH)CH₃, —CH(OH)CF₃, —C(OH)(CF₃)₂, —CH(OH)CH₃, —CH(NH₂)C(CH₃)₃, —CH₂C(O)OC(CH₃)₃, and —C(CH₃)(NH₂)CF₃, —CH₂CH(OH)(C₆H₅), —CH₂C(OH)(CH₃)CF₃, —CH₂C(OH)(CH₃)(C₃H₅), —CH₂CH₂(C₅H₄N), —CH₂CH(OH)(C₆H₁₁), —CH₂C(OH)(CH₃)₂, —CH₂CH(NH₂)(C₆H₅), —CH₂C(OH)(CF₃)₂, —CH₂—C(CH₃)₂NHCOCH₃, —CH₂—C(CH₃)₂NHSO₂CH₃, —CH₂—CH(OH)—C(CH₃)(C₃H₄), —CH₂—C(CH₃)₂CONH₂, —CH₂—C(CH₃)₂CON(CH₃)₂, and —CH₂—C(CH₃)₂SO₂CH₃.

In some embodiments, provided is a compound of formula (I), or a stereoisomer, tautomer, pharmaceutically acceptable salt or solvate thereof, wherein R² is pyridinyl or pyrazolyl optionally substituted with one or more substituents independently selected from the group consisting of —CF₃ and —CH₃.

In some embodiments, provided is a compound of formula (I), or a stereoisomer, tautomer, pharmaceutically acceptable salt or solvate thereof, wherein R² is pyridinyl substituted with —CF₃.

In some embodiments, provided is a compound of formula (I), or a stereoisomer, tautomer, pharmaceutically acceptable salt or solvate thereof, wherein R² is pyridinyl.

In some embodiments, provided is a compound of formula (I), or a stereoisomer, tautomer, pharmaceutically acceptable salt or solvate thereof, wherein R² is pyrazolyl substituted with —CH₃.

In some embodiments, provided is a compound of formula (I), or a stereoisomer, tautomer, pharmaceutically acceptable salt or solvate thereof, wherein R² is pyrazolyl.

In some embodiments, provided is a compound of formula (I), or a stereoisomer, tautomer, pharmaceutically acceptable salt or solvate thereof, wherein R² is:

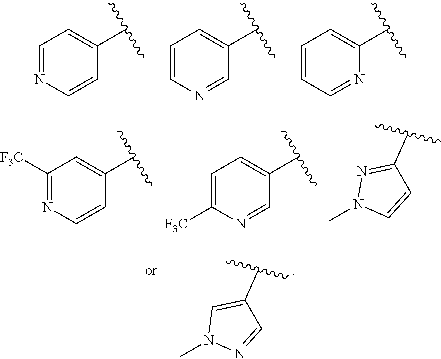

In some embodiments, provided is a compound of formula (I), or a stereoisomer, tautomer, pharmaceutically acceptable salt or solvate thereof, wherein Z is —O—Cₙ₁H₂ₙ₁—, provided that O is directly bonded to the pyridinyl ring or phenyl ring or pyridazinyl ring. In one embodiment, n1 is 0, and thus Z is —O—. In another embodiment, n1 is 1, and thus Z is —OCH₂—. In another embodiment, n1 is 2, and thus Z is —O—CH(CH₃)— or —O—CH₂—CH₂—.

In some embodiments, provided is a compound of formula (I), or a stereoisomer, tautomer, pharmaceutically acceptable salt or solvate thereof, wherein Z is —NR⁶—(CH₂)ₙ₂—, provided that N is directly bonded to the pyridinyl or phenyl ring or pyridazinyl ring. In one embodiment, n2 is 0, and thus Z is —NR⁶—. In another embodiment, n2 is 1, and thus Z is —NR⁶—(CH₂)—.

In some embodiments, provided is a compound of formula (I), or a stereoisomer, tautomer, pharmaceutically acceptable salt or solvate thereof, wherein R⁶ is hydrogen.

In some embodiments, provided is a compound of formula (I), or a stereoisomer, tautomer, pharmaceutically acceptable salt or solvate thereof, wherein R² and R⁶ are taken together to form a 4- to 8-membered monocyclic or bicyclic heterocyclyl selected from the group consisting of:

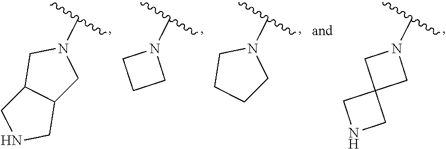 [SMILES: *N1CC2CNCC2C1 ; *N1CCC1 ; *N1CCCC1 ; *N1CC2(CNC2)C1]

wherein the 4- to 8-membered monocyclic or bicyclic heterocyclyl is optionally substituted with one or more substituents independently selected from the group consisting —OH, —CH₂OH, —CH₂N(R¹⁰)₂, —C(CH₃)₂OH, —CF₃, and —C(O)N(R¹⁰)₂; and each R¹⁰ is independently hydrogen or —CH₃.

In some embodiments, provided is a compound of formula (I), or a stereoisomer, tautomer, pharmaceutically acceptable salt or solvate thereof, wherein R² and R⁶ are taken together to form a 4- to 8-membered monocyclic or bicyclic heterocyclyl selected from the group consisting of:

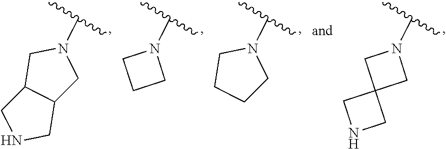 [SMILES: *N1CC2CNCC2C1 ; *N1CCC1 ; *N1CCCC1 ; *N1CC2(CNC2)C1]

wherein the 4- to 8-membered monocyclic or bicyclic heterocyclyl is optionally substituted with one or more substituents independently selected from the group consisting —OH, —CH₂OH, —CH₂N(CH₃)₂, —C(CH₃)₂OH, —CF₃, and —C(O)NHCH₃.

In some embodiments, provided is a compound of formula (I), or a stereoisomer, tautomer, pharmaceutically acceptable salt or solvate thereof, wherein R² and R⁶ are taken together to form a 4- to 8-membered monocyclic or bicyclic heterocyclyl selected from the group consisting of:

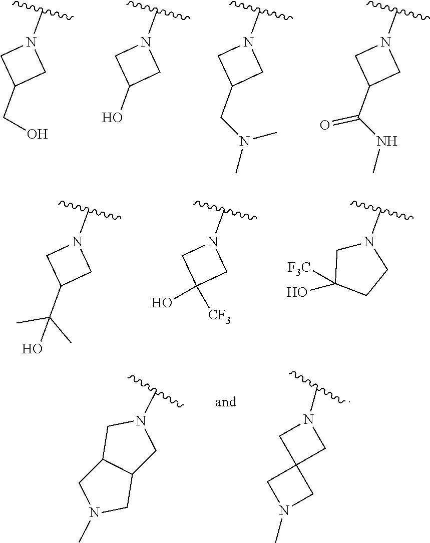

In some embodiments, provided is a compound of formula (I), or a stereoisomer, tautomer, pharmaceutically acceptable salt or solvate thereof, wherein each R⁸ is independently hydrogen, —CH₃, —C(CH₃)₃, cyclopropyl, —CH₂CH₃, —CH₂CF₃, —CH₂CH₂OH, —CH₂C(OH)(CH₃)₂, —CH₂CH₂OCH₃, —CH₂C(CN)(CH₃)₂,

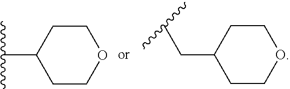 [SMILES: *C1CCOCC1 ; *CC1CCOCC1]

In some embodiments, provided is a compound of formula (I), or a stereoisomer, tautomer, pharmaceutically acceptable salt or solvate thereof, wherein R⁹ is —CF₃.

In some embodiments, provided is a compound of formula (I), or a stereoisomer, tautomer, pharmaceutically acceptable salt or solvate thereof, wherein R⁹ is —CH₃.

In some embodiments, provided is a compound of formula (I), or a stereoisomer, tautomer, pharmaceutically acceptable salt or solvate thereof, wherein R¹¹ is hydrogen, —CH₃, —CH₂CH₃, —CH₂(C₃H₅), —CH₂CH₂—OCHF₂, or cyclopropyl.

In some embodiments, provided is a compound of formula (I), or a stereoisomer, tautomer, pharmaceutically acceptable salt or solvate thereof, wherein R¹² is hydrogen, —CH₃, —C₃H₅, —SO₂CH₃, —CH₂CN, —CH₂(C₆H₅), —CH₂COOCH₂CH₃, —C₅H₄N, and

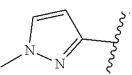 [SMILES: *c1ccn(C)n1]

In some embodiments, provided is a compound of formula (I), or a stereoisomer, tautomer, pharmaceutically acceptable salt or solvate thereof, wherein R¹³ is hydrogen or methyl.

In some embodiments, provided is a compound of formula (I), or a stereoisomer, tautomer, pharmaceutically acceptable salt or solvate thereof, wherein R¹⁴ is methyl or cyclopropyl.

In some embodiments, provided is a compound of formula (I), or a stereoisomer, tautomer, pharmaceutically acceptable salt or solvate thereof, wherein R¹⁵ is hydrogen, —CH₃, —CH₂CH₃, —CH₂CH(CH₃)₂, —CH₂CH₂OH, —CUE, —CH₂CF₃, —CH₂CHF₂, —CH₂CH₂F, —CH₂CN, or —CH(CN)CH₃.

In some embodiments, the compound of formula (I) is a compound of formula (I-A):

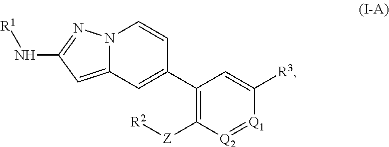

or a stereoisomer, tautomer, pharmaceutically acceptable salt or solvate thereof, wherein Z, Q₁, Q₂, R¹, R², and R³ are defined as in formula (I).

In some embodiments, provided is a compound of formula (I-A), or a stereoisomer, tautomer, pharmaceutically acceptable salt or solvate thereof, wherein Q₁ is N, and Q₂ is CH.

In some embodiments, provided is a compound of formula (I-A), or a stereoisomer, tautomer, pharmaceutically acceptable salt or solvate thereof, wherein Q₁ is CH, and Q₂ is CH.

In some embodiments, provided is a compound of formula (I-A), or a stereoisomer, tautomer, pharmaceutically acceptable salt or solvate thereof, wherein Q₁ is N, and Q₂ is N.

In some embodiments, the compound of formula (I) is a compound of formula (I-B):

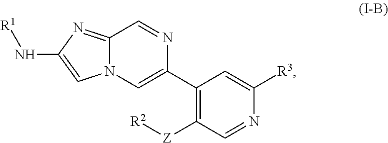

or a stereoisomer, tautomer, pharmaceutically acceptable salt or solvate thereof, wherein Z, R¹, R², and R³ are defined as in formula (I).

All possible combinations of the above-indicated embodiments of compounds of formula (I) and their tautomers, stereoisomers, pharmaceutically acceptable salts and solvates are considered to be embraced within the scope of this application.

Exemplary compounds of formula (I) include, but are not limited to:
N-[5-[2-Cyano-5-[[(3R)-1-methylpyrrolidin-3-yl]methoxy]-4-pyridyl]pyrazolo[1,5-a]pyridin-2-yl]cyclopropanecarboxamide;
N-[5-[2-cyano-5-(2-dimethylaminoethylamino)-4-pyridyl]pyrazolo[1,5-a]pyridin-2-yl]cyclopropanecarboxamide;
N-[5-[2-cyano-5-[3-(hydroxymethyl)azetidin-1-yl]-4-pyridyl]pyrazolo[1,5-a]pyridin-2-yl]cyclopropanecarboxamide;
(R)-5-((1-methylpyrrolidin-3-yl)methoxy)-4-(2-(pyridin-3-ylamino)pyrazolo[1,5-a]pyridin-5-yl)picolinonitrile;
N-[5-[2-cyano-5-[(3S)-1-(2,2,2-trifluoroethyl)pyrrolidin-3-yl]oxy-4-pyridyl]pyrazolo[1,5-a]pyridin-2-yl]cyclopropanecarboxamide;
(S)—N-(5-(2-cyano-5-(pyrrolidin-3-yloxy)pyridin-4-yl)pyrazolo[1,5-a]pyridin-2-yl)cyclopropanecarboxamide
N-[5-[2-cyano-5-[(3S)-1-cyclopropylpyrrolidin-3-yl]oxy-4-pyridyl]pyrazolo[1,5-a]pyridin-2-yl]cyclopropanecarboxamide
N-(5-(2-cyano-5-(((2*S,4*R)-2-(trifluoromethyl)piperidin-4-yl)oxy)pyridin-4-yl)pyrazolo[1,5-a]pyridin-2-yl)cyclopropanecarboxamide
N-(5-(2-cyano-5-((4-(trifluoromethyl)piperidin-4-yl)oxy)pyridin-4-yl)pyrazolo[1,5-a]pyridin-2-yl)cyclopropanecarboxamide;
N-[5-[2-cyano-5-[[1-methyl-4-(trifluoromethyl)-4-piperidyl]oxy]-4-pyridyl]pyrazolo[1,5-a]pyridin-2-yl]cyclopropanecarboxamide;
N-[5-[2-cyano-5-[[(4R)-3,3-difluoro-1-methyl-4-piperidyl]oxy]-4-pyridyl]pyrazolo[1,5-a]pyridin-2-yl]cyclopropanecarboxamide;
N-[5-[2-cyano-5-[[(6R)-4-methyl-1,4-oxazepan-6-yl]oxy]-4-pyridyl]pyrazolo[1,5-a]pyridin-2-yl]cyclopropanecarboxamide;
N-[5-[2-cyano-5-[[(1R,4R,5R)-2-methyl-2-azabicyclo[2.2.1]heptan-5-yl]oxy]-4-pyridyl]pyrazolo[1,5-a]pyridin-2-yl]cyclopropanecarboxamide;
N-(5-(2-cyano-5-(((2*R,4*S)-2-(trifluoromethyl)piperidin-4-yl)oxy)pyridin-4-yl)pyrazolo[1,5-a]pyridin-2-yl)cyclopropanecarboxamide;
N-[5-[2-cyano-5-[[(4R)-3,3-difluoro-4-piperidyl]oxy]-4-pyridyl]pyrazolo[1,5-a]pyridin-2-yl]cyclopropanecarboxamide;
(S)—N-(5-(2-cyano-5-((1-methylpyrrolidin-3-yl)methoxy)pyridin-4-yl)pyrazolo[1,5-a]pyridin-2-yl)cyclopropanecarboxamide;
N-[5-[5-[[(1*S,4*S,5*R)-2-azabicyclo[2.2.1]heptan-5-yl]oxy]-2-cyano-4-pyridyl]pyrazolo[1,5-a]pyridin-2-yl]cyclopropanecarboxamide;
N-[5-[5-[[(1*R,4*R,5*S)-2-azabicyclo[2.2.1]heptan-5-yl]oxy]-2-cyano-4-pyridyl]pyrazolo[1,5-a]pyridin-2-yl]cyclopropanecarboxamide;
N-[5-[2-cyano-5-[[(2S)-morpholin-2-yl]methoxy]-4-pyridyl]pyrazolo[1,5-a]pyridin-2-yl]cyclopropanecarboxamide;
N-[5-[5-[[(1R,3S)-3-aminocyclopentyl]methoxy]-2-cyano-4-pyridyl]pyrazolo[1,5-a]pyridin-2-yl]cyclopropanecarboxamide;
N-[5-[2-cyano-5-[[(6R)-1,4-oxazepan-6-yl]oxy]-4-pyridyl]pyrazolo[1,5-a]pyridin-2-yl]cyclopropanecarboxamide;
N-[5-[5-[[(1R,3R)-3-aminocyclopentyl]methoxy]-2-cyano-4-pyridyl]pyrazolo[1,5-a]pyridin-2-yl]cyclopropanecarboxamide;
N-[5-[5-(azetidin-3-ylmethoxy)-2-cyano-4-pyridyl]pyrazolo[1,5-a]pyridin-2-yl]cyclopropanecarboxamide;
N-[5-[5-[[(1R,4R,5R)-2-azabicyclo[2.2.1]heptan-5-yl]oxy]-2-cyano-4-pyridyl]pyrazolo[1,5-a]pyridin-2-yl]cyclopropanecarboxamide;
N-[5-[2-cyano-5-[(3R)-1-cyclopropylpyrrolidin-3-yl]oxy-4-pyridyl]pyrazolo[1,5-a]pyridin-2-yl]cyclopropanecarboxamide;
N-[5-[2-cyano-5-[[(6S)-4-methyl-1,4-oxazepan-6-yl]oxy]-4-pyridyl]pyrazolo[1,5-a]pyridin-2-yl]cyclopropanecarboxamide;
N-[5-[2-cyano-5-[[(1S,5R)-8-methyl-8-azabicyclo[3.2.1]octan-3-yl]methoxy]-4-pyridyl]pyrazolo[1,5-a]pyridin-2-yl]cyclopropanecarboxamide;
N-[5-[2-cyano-5-[[(3S,5R)-1-methyl-5-(trifluoromethyl)-3-piperidyl]oxy]-4-pyridyl]pyrazolo[1,5-a]pyridin-2-yl]cyclopropanecarboxamide;
N-(5-(2-cyano-5-(((2*S,4*R)-1-methyl-2-(trifluoromethyl)piperidin-4-yl)oxy)pyridin-4-yl)pyrazolo[1,5-a]pyridin-2-yl)cyclopropanecarboxamide;
N-(5-(2-cyano-5-(((2*R,4*S)-1-methyl-2-(trifluoromethyl)piperidin-4-yl)oxy)pyridin-4-yl)pyrazolo[1,5-a]pyridin-2-yl)cyclopropanecarboxamide;
N-[5-[5-(azetidin-3-yloxy)-2-cyano-4-pyridyl]pyrazolo[1,5-a]pyridin-2-yl]cyclopropanecarboxamide;
N-[5-[2-cyano-5-[(1-methylazetidin-3-yl)methoxy]-4-pyridyl]pyrazolo[1,5-a]pyridin-2-yl]cyclopropanecarboxamide;
rac-N-(5-(2-cyano-5-((5-oxopyrrolidin-3-yl)methoxy)pyridin-4-yl)pyrazolo[1,5-a]pyridin-2-yl)cyclopropanecarboxamide;
tert-butyl 2-[[6-cyano-4-[2-(cyclopropanecarbonylamino)pyrazolo[1,5-a]pyridin-5-yl]-3-pyridyl]oxy]-6-azaspiro[3.3]heptane-6-carboxylate;
tert-butyl (3R)-3-[[6-cyano-4-[2-(cyclopropanecarbonylamino)pyrazolo[1,5-a]pyridin-5-yl]-3-pyridyl]oxymethyl]pyrrolidine-1-carboxylate;
(R)—N-(5-(2-cyano-5-((4-methylmorpholin-2-yl)methoxy)pyridin-4-yl)pyrazolo[1,5-a]pyridin-2-yl)cyclopropanecarboxamide;
(R)—N-(5-(2-cyano-5-((1-methylpiperidin-3-yl)methoxy)pyridin-4-yl)pyrazolo[1,5-a]pyridin-2-yl)cyclopropanecarboxamide;
(S)—N-(5-(2-cyano-5-((1-methylpiperidin-3-yl)methoxy)pyridin-4-yl)pyrazolo[1,5-a]pyridin-2-yl)cyclopropanecarboxamide;
N-[5-[2-cyano-5-[[(2S)-4-methylmorpholin-2-yl]methoxy]-4-pyridyl]pyrazolo[1,5-a]pyridin-2-yl]cyclopropanecarboxamide;
N-[5-[2-cyano-5-[(2-oxo-3-azabicyclo[2.2.2]octan-4-yl)methoxy]-4-pyridyl]pyrazolo[1,5-a]pyridin-2-yl]cyclopropanecarboxamide;
N-[5-[2-cyano-5-[(1-hydroxycyclobutyl)methoxy]-4-pyridyl]pyrazolo[1,5-a]pyridin-2-yl]cyclopropanecarboxamide;
N-[5-[2-cyano-5-[(1-hydroxycyclopentyl)methoxy]-4-pyridyl]pyrazolo[1,5-a]pyridin-2-yl]cyclopropanecarboxamide;
N-[5-[2-cyano-5-(4-piperidylmethoxy)-4-pyridyl]pyrazolo[1,5-a]pyridin-2-yl]cyclopropanecarboxamide;
N-[5-[5-[(1-aminocyclobutyl)methoxy]-2-cyano-4-pyridyl]pyrazolo[1,5-a]pyridin-2-yl]cyclopropanecarboxamide;
N-[5-[2-Cyano-5-[[(3R)-pyrrolidin-3-yl]methoxy]-4-pyridyl]pyrazolo[1,5-a]pyridin-2-yl]cyclopropanecarboxamide;
N-[5-[2-Cyano-5-(cyclopropoxy)-4-pyridyl]pyrazolo[1,5-a]pyridin-2-yl]cyclopropanecarboxamide;
N-[5-[2-Cyano-5-(3-pyridylmethoxy)-4-pyridyl]pyrazolo[1,5-a]pyridin-2-yl]cyclopropanecarboxamide;
N-(5-(2-Cyano-5-(pyridin-2-ylmethoxy)pyridin-4-yl)pyrazolo[1,5-a]pyridin-2-yl)cyclopropanecarboxamide;
(*R)—N-(5-(2-Cyano-5-(3,3,3-trifluoro-2-hydroxy-2-methylpropoxy)pyridin-4-yl)pyrazolo[1,5-a]pyridin-2-yl)cyclopropanecarboxamide;
(*S)—N-(5-(2-Cyano-5-(3,3,3-trifluoro-2-hydroxy-2-methylpropoxy)pyridin-4-yl)pyrazolo[1,5-a]pyridin-2-yl)cyclopropanecarboxamide;
N-[5-[2-cyano-5-[(1-methyl-4-piperidyl)methoxy]-4-pyridyl]pyrazolo[1,5-a]pyridin-2-yl]cyclopropanecarboxamide;
N-[5-[2-Cyano-5-(2-hydroxy-2-methyl-propoxy)-4-pyridyl]pyrazolo[1,5-a]pyridin-2-yl]cyclopropanecarboxamide;
trans-N-(5-(2-Cyano-5-(((1r,4r)-4-hydroxycyclohexyl)methoxy)pyridin-4-yl)pyrazolo[1,5-a]pyridin-2-yl)cyclopropanecarboxamide;
N-[5-[2-Cyano-5-(1-methylazetidin-3-yl)oxy-4-pyridyl]pyrazolo[1,5-a]pyridin-2-yl]cyclopropanecarboxamide;
tert-butyl 2-[[6-Cyano-4-[2-(cyclopropanecarbonylamino)pyrazolo[1,5-a]pyridin-5-yl]-3-pyridyl]oxy]acetate;
(*R)—N-(5-(5-((1-Acetylpyrrolidin-3-yl)oxy)-2-cyanopyridin-4-yl)pyrazolo[1,5-a]pyridin-2-yl)cyclopropanecarboxamide;
(*S)—N-(5-(5-((1-Acetylpyrrolidin-3-yl)oxy)-2-cyanopyridin-4-yl)pyrazolo[1,5-a]pyridin-2-yl)cyclopropanecarboxamide;
(*R)—N-(5-(2-cyano-5-((1-methylpyrrolidin-3-yl)oxy)pyridin-4-yl)pyrazolo[1,5-a]pyridin-2-yl)cyclopropanecarboxamide;
(*S)—N-(5-(2-cyano-5-((1-methylpyrrolidin-3-yl)oxy)pyridin-4-yl)pyrazolo[1,5-a]pyridin-2-yl)cyclopropanecarboxamide;
N-[5-[5-[(1-acetyl-4-piperidyl)methoxy]-2-cyano-4-pyridyl]pyrazolo[1,5-a]pyridin-2-yl]cyclopropanecarboxamide;
N-[5-[2-cyano-5-[[6-(trifluoromethyl)-3-pyridyl]methoxy]-4-pyridyl]pyrazolo[1,5-a]pyridin-2-yl]cyclopropanecarboxamide;
N-[5-[2-cyano-5-[[2-(trifluoromethyl)-4-pyridyl]methoxy]-4-pyridyl]pyrazolo[1,5-a]pyridin-2-yl]cyclopropanecarboxamide;
N-[5-[2-cyano-5-[(2S)-2-hydroxypropoxy]-4-pyridyl]pyrazolo[1,5-a]pyridin-2-yl]cyclopropanecarboxamide;
N-[5-[2-cyano-5-(4-hydroxycyclohexoxy)-4-pyridyl]pyrazolo[1,5-a]pyridin-2-yl]cyclopropanecarboxamide;
N-[5-[2-cyano-5-[[(3S)-1-methyl-3-piperidyl]oxy]-4-pyridyl]pyrazolo[1,5-a]pyridin-2-yl]cyclopropanecarboxamide;
N-[5-[2-cyano-5-[[(1S,5R)-8-methyl-8-azabicyclo[3.2.1]octan-3-yl]oxy]-4-pyridyl]pyrazolo[1,5-a]pyridin-2-yl]cyclopropanecarboxamide;
N-[5-[5-[[(1S,5R)-8-azabicyclo[3.2.1]octan-3-yl]methoxy]-2-cyano-4-pyridyl]pyrazolo[1,5-a]pyridin-2-yl]cyclopropanecarboxamide;
N-[5-[2-cyano-5-[(3-hydroxycyclobutyl)methoxy]-4-pyridyl]pyrazolo[1,5-a]pyridin-2-yl]cyclopropanecarboxamide;
N-[5-[2-cyano-5-[3-(hydroxymethyl)cyclobutoxy]-4-pyridyl]pyrazolo[1,5-a]pyridin-2-yl]cyclopropanecarboxamide;
N-[5-[2-cyano-5-(3-hydroxyazetidin-1-yl)-4-pyridyl]pyrazolo[1,5-a]pyridin-2-yl]cyclopropanecarboxamide;
N-[5-[2-cyano-5-[[(3R)-1-methylpyrrolidin-3-yl]methylamino]-4-pyridyl]pyrazolo[1,5-a]pyridin-2-yl]cyclopropanecarboxamide;
N-[5-[2-cyano-5-[3-(dimethylaminomethyl)azetidin-1-yl]-4-pyridyl]pyrazolo[1,5-a]pyridin-2-yl]cyclopropanecarboxamide;
1-[6-cyano-4-[2-(cyclopropanecarbonylamino)pyrazolo[1,5-a]pyridin-5-yl]-3-pyridyl]-N-methyl-azetidine-3-carboxamide;
N-[5-[2-cyano-5-[3-(1-hydroxy-1-methyl-ethyl)azetidin-1-yl]-4-pyridyl]pyrazolo[1,5-a]pyridin-2-yl]cyclopropanecarboxamide;
N-[5-[2-cyano-5-[3-hydroxy-3-(trifluoromethyl)azetidin-1-yl]-4-pyridyl]pyrazolo[1,5-a]pyridin-2-yl]cyclopropanecarboxamide;
N-[5-[2-cyano-5-(cyclopropylmethylamino)-4-pyridyl]pyrazolo[1,5-a]pyridin-2-yl]cyclopropanecarboxamide;
N-[5-[2-cyano-5-[(1-methylpyrazol-3-yl)amino]-4-pyridyl]pyrazolo[1,5-a]pyridin-2-yl]cyclopropanecarboxamide;
N-[5-[2-cyano-5-(cyclopentylamino)-4-pyridyl]pyrazolo[1,5-a]pyridin-2-yl]cyclopropanecarboxamide;
N-[5-[2-cyano-5-[(1-methylpyrazol-4-yl)amino]-4-pyridyl]pyrazolo[1,5-a]pyridin-2-yl]cyclopropanecarboxamide;
N-[5-[2-cyano-5-(cyclopropylamino)-4-pyridyl]pyrazolo[1,5-a]pyridin-2-yl]cyclopropanecarboxamide;
N-[5-[2-cyano-5-[(2-hydroxy-2-methyl-propyl)amino]-4-pyridyl]pyrazolo[1,5-a]pyridin-2-yl]cyclopropanecarboxamide;
(3-endo)-N-(5-(5-(((1R,3r,5S)-8-azabicyclo[3.2.1]octan-3-yl)oxy)-2-cyanopyridin-4-yl)pyrazolo[1,5-a]pyridin-2-yl)cyclopropanecarboxamide;
N-(5-(2-cyano-5-(((1R,3R,5S)-8-methyl-8-azabicyclo[3.2.1]octan-3-yl)methoxy)pyridin-4-yl)pyrazolo[1,5-a]pyridin-2-yl)cyclopropanecarboxamide;
N-[5-[2-cyano-5-[[(6S)-1,4-oxazepan-6-yl]oxy]-4-pyridyl]pyrazolo[1,5-a]pyridin-2-yl]cyclopropanecarboxamide;
N-[5-[2-cyano-5-[(1-methyl-4-piperidyl)oxy]-4-pyridyl]pyrazolo[1,5-a]pyridin-2-yl]cyclopropanecarboxamide;
N-(5-(2-cyano-5-(((1s,4s)-4-hydroxycyclohexyl)oxy)pyridin-4-yl)pyrazolo[1,5-a]pyridin-2-yl)cyclopropanecarboxamide;
(rac-)-N-[5-[2-cyano-5-[3-hydroxy-3-(trifluoromethyl)pyrrolidin-1-yl]-4-pyridyl]pyrazolo[1,5-a]pyridin-2-yl]cyclopropanecarboxamide;
(R)—N-(5-(2-cyano-5-((1-methylpiperidin-3-yl)oxy)pyridin-4-yl)pyrazolo[1,5-a]pyridin-2-yl)cyclopropanecarboxamide;
cis-N-(5-(2-cyano-5-(((1S,4S)-4-hydroxy-4-methylcyclohexyl)oxy)pyridin-4-yl)pyrazolo[1,5-a]pyridin-2-yl)cyclopropanecarboxamide;
N-[5-[2-cyano-5-(2-methyl-1,3,3a,4,6,6a-hexahydropyrrolo[3,4-c]pyrrol-5-yl)-4-pyridyl]pyrazolo[1,5-a]pyridin-2-yl]cyclopropanecarboxamide;
N-[5-[2-cyano-5-(2-methyl-2,6-diazaspiro[3.3]heptan-6-yl)-4-pyridyl]pyrazolo[1,5-a]pyridin-2-yl]cyclopropanecarboxamide;
(3-endo)-N-[5-[2-cyano-5-[[(1R,5S)-3-oxa-9-azabicyclo[3.3.1]nonan-7-yl]oxy]-4-pyridyl]pyrazolo[1,5-a]pyridin-2-yl]cyclopropanecarboxamide;
N-[5-[2-cyano-5-[(2,2,6,6-tetramethyl-4-piperidyl)oxy]-4-pyridyl]pyrazolo[1,5-a]pyridin-2-yl]cyclopropanecarboxamide;
N-[5-[2-cyano-5-[(2R)-2-hydroxypropoxy]-4-pyridyl]pyrazolo[1,5-a]pyridin-2-yl]cyclopropanecarboxamide;
N-[5-[2-cyano-5-[[(3S,5R)-5-(trifluoromethyl)-3-piperidyl]oxy]-4-pyridyl]pyrazolo[1,5-a]pyridin-2-yl]cyclopropanecarboxamide;
N-[5-[2-cyano-5-[(2R)-3,3,3-trifluoro-2-hydroxy-propoxy]-4-pyridyl]pyrazolo[1,5-a]pyridin-2-yl]cyclopropanecarboxamide;
N-[5-[2-cyano-5-[(2S)-3,3,3-trifluoro-2-hydroxy-propoxy]-4-pyridyl]pyrazolo[1,5-a]pyridin-2-yl]cyclopropanecarboxamide;
N-(5-(2-cyano-5-(((1s,3s)-3-(methylamino)cyclobutyl)methoxy)pyridin-4-yl)pyrazolo[1,5-a]pyridin-2-yl)cyclopropanecarboxamide;
N-[5-[2-cyano-5-[3,3,3-trifluoro-2-hydroxy-2-(trifluoromethyl)propoxy]-4-pyridyl]pyrazolo[1,5-a]pyridin-2-yl]cyclopropanecarboxamide;
N-[5-[5-cyano-2-[[(3R)-1-methylpyrrolidin-3-yl]methoxy]phenyl]pyrazolo[1,5-a]pyridin-2-yl]cyclopropanecarboxamide;
N-[5-[5-cyano-2-[[(2R)-morpholin-2-yl]methoxy]phenyl]pyrazolo[1,5-a]pyridin-2-yl]cyclopropanecarboxamide;
N-[5-[5-cyano-2-[[(2R)-6,6-dimethylmorpholin-2-yl]methoxy]phenyl]pyrazolo[1,5-a]pyridin-2-yl]cyclopropanecarboxamide;
N-[5-[5-cyano-2-[[(2S)-morpholin-2-yl]methoxy]phenyl]pyrazolo[1,5-a]pyridin-2-yl]cyclopropanecarboxamide;
N-[5-[5-cyano-2-[[(2S)-6,6-dimethylmorpholin-2-yl]methoxy]phenyl]pyrazolo[1,5-a]pyridin-2-yl]cyclopropanecarboxamide;
5-[[(3R)-1-methylpyrrolidin-3-yl]methoxy]-4-[2-(4-pyridylamino)pyrazolo[1,5-a]pyridin-5-yl]pyridine-2-carbonitrile;
4-(2-anilinopyrazolo[1,5-a]pyridin-5-yl)-5-[[(3R)-1-methylpyrrolidin-3-yl]methoxy]pyridine-2-carbonitrile;
5-[[(3R)-1-methylpyrrolidin-3-yl]methoxy]-4-[2-(2-pyridylamino)pyrazolo[1,5-a]pyridin-5-yl]pyridine-2-carbonitrile;
(S)—N-[5-[2-cyano-5-[(3S)-1-(2-methoxyethyl)pyrrolidin-3-yl]oxy-4-pyridyl]pyrazolo[1,5-a]pyridin-2-yl]cyclopropanecarboxamide;
(S)—N-[5-[2-cyano-5-[(3S)-1-(2-pyridylmethyl)pyrrolidin-3-yl]oxy-4-pyridyl]pyrazolo[1,5-a]pyridin-2-yl]cyclopropanecarboxamide;
(R)—N-[5-[2-cyano-5-[(3R)-1-(2,2,2-trifluoroethyl)pyrrolidin-3-yl]oxy-4-pyridyl]pyrazolo[1,5-a]pyridin-2-yl]cyclopropanecarboxamide;
(R)—N-[5-[2-cyano-5-[(3R)-1-(2-methoxyethyl)pyrrolidin-3-yl]oxy-4-pyridyl]pyrazolo[1,5-a]pyridin-2-yl]cyclopropanecarboxamide;
(R)—N-[5-[2-cyano-5-[(3R)-1-(2-pyridylmethyl)pyrrolidin-3-yl]oxy-4-pyridyl]pyrazolo[1,5-a]pyridin-2-yl]cyclopropanecarboxamide;
(R)—N-[5-[2-cyano-5-[(3R)-1-(cyanomethyl)pyrrolidin-3-yl]oxy-4-pyridyl]pyrazolo[1,5-a]pyridin-2-yl]cyclopropanecarboxamide;
(S)—N-[5-[2-cyano-5-[(3S)-1-(cyanomethyl)pyrrolidin-3-yl]oxy-4-pyridyl]pyrazolo[1,5-a]pyridin-2-yl]cyclopropanecarboxamide;
N-(5-(2-methyl-5-((1-methylazetidin-3-yl)oxy)pyridin-4-yl)pyrazolo[1,5-a]pyridin-2-yl)cyclopropanecarboxamide;
N-(1-(difluoromethyl)-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine;
N-(4-fluoro-1-methyl-1H-pyrazol-3-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
N-(3-fluoro-1-methyl-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
N-(5-fluoro-1-methyl-1H-pyrazol-3-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
1-(4-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-1H-pyrazol-1-yl)-2-methylpropan-2-ol;
1-(4-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-1-(2-hydroxy-2-methylpropyl)-1H-pyrazol-3-yl)-2,2,2-trifluoroethan-1-one;
7-endo-5-[2-methyl-5-[[(1S,5R)-3-oxa-9-azabicyclo[3.3.1]nonan-7-yl]oxy]-4-pyridyl]-N-(1-methylpyrazol-4-yl)pyrazolo[1,5-a]pyridin-2-amine;
7-endo-N-(1-methyl-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
endo-5-(5-(((1R,5S,7s)-3-oxa-9-azabicyclo[3.3.1]nonan-7-yl)oxy)-2-methylpyridin-4-yl)-N-(1-methyl-1H-pyrazol-3-yl)pyrazolo[1,5-a]pyridin-2-amine;
7-endo-N-(5-fluoropyridin-2-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
5-(5-(((1R,5S,7s)-3-oxa-9-azabicyclo[3.3.1]nonan-7-yl)oxy)-2-methylpyridin-4-yl)-N-(5-fluoropyridin-2-yl)pyrazolo[1,5-a]pyridin-2-amine;
5-[2-methyl-5-[[(2S)-morpholin-2-yl]methoxy]-4-pyridyl]-N-(1-methylpyrazol-4-yl)pyrazolo[1,5-a]pyridin-2-amine;
5-[2-methyl-5-[[(2S)-morpholin-2-yl]methoxy]-4-pyridyl]-N-(1-methylpyrazol-3-yl)pyrazolo[1,5-a]pyridin-2-amine;
5-[2-methyl-5-[[(2S)-morpholin-2-yl]methoxy]-4-pyridyl]-N-(2-pyridyl)pyrazolo[1,5-a]pyridin-2-amine;
5-[2-methyl-5-[[(2S)-morpholin-2-yl]methoxy]-4-pyridyl]-N-(3-pyridyl)pyrazolo[1,5-a]pyridin-2-amine;
5-[2-methyl-5-[[(2S)-morpholin-2-yl]methoxy]-4-pyridyl]-N-pyrimidin-4-yl-pyrazolo[1,5-a]pyridin-2-amine;
N-(1-isopropylpyrazol-4-yl)-5-[2-methyl-5-[[(2S)-morpholin-2-yl]methoxy]-4-pyridyl]pyrazolo[1,5-a]pyridin-2-amine;
5-[2-methyl-5-[[(2S)-morpholin-2-yl]methoxy]-4-pyridyl]-N-pyridazin-3-yl-pyrazolo[1,5-a]pyridin-2-amine;
N-(1-isopropylpyrazol-3-yl)-5-[2-methyl-5-[[(2S)-morpholin-2-yl]methoxy]-4-pyridyl]pyrazolo[1,5-a]pyridin-2-amine;
endo-N-(5-(5-(((1R,5S,7s)-3-oxa-9-azabicyclo[3.3.1]nonan-7-yl)oxy)-2-methylpyridin-4-yl)pyrazolo[1,5-a]pyridin-2-yl)cyclopropanecarboxamide;
N-[5-[2-methyl-5-[(3R)-1-methylpyrrolidin-3-yl]oxy-4-pyridyl]pyrazolo[1,5-a]pyridin-2-yl]cyclopropanecarboxamide;
N-(5-(2-methyl-5-(((1R,3r,5S)-8-methyl-8-azabicyclo[3.2.1]octan-3-yl)oxy)pyridin-4-yl)pyrazolo[1,5-a]pyridin-2-yl)cyclopropanecarboxamide;
N-[5-[2-methyl-5-[(3S)-1-methylpyrrolidin-3-yl]oxy-4-pyridyl]pyrazolo[1,5-a]pyridin-2-yl]cyclopropanecarboxamide;
N-(5-(2-methyl-5-(((1R,3r,5S)-8-methyl-8-azabicyclo[3.2.1]octan-3-yl)oxy)pyridin-4-yl)pyrazolo[1,5-a]pyridin-2-yl)cyclopropanecarboxamide;
N-[5-[5-(1-ethylazetidin-3-yl)oxy-2-methyl-4-pyridyl]pyrazolo[1,5-a]pyridin-2-yl]cyclopropanecarboxamide;
N-[5-[5-(1-isopropylazetidin-3-yl)oxy-2-methyl-4-pyridyl]pyrazolo[1,5-a]pyridin-2-yl]cyclopropanecarboxamide;
N-[5-[2-methyl-5-[[(2S)-morpholin-2-yl]methoxy]-4-pyridyl]pyrazolo[1,5-a]pyridin-2-yl]cyclopropanecarboxamide;
N-[5-[5-[(3-aminooxetan-3-yl)methoxy]-2-methyl-4-pyridyl]pyrazolo[1,5-a]pyridin-2-yl]cyclopropanecarboxamide;
N-[5-[5-[(1-amino-3,3-difluoro-cyclobutyl)methoxy]-2-methyl-4-pyridyl]pyrazolo[1,5-a]pyridin-2-yl]cyclopropanecarboxamide;
trans-N-[5-[5-(3-methoxycyclobutoxy)-2-methyl-4-pyridyl]pyrazolo[1,5-a]pyridin-2-yl]cyclopropanecarboxamide;
cis-N-[5-[5-(3-methoxycyclobutoxy)-2-methyl-4-pyridyl]pyrazolo[1,5-a]pyridin-2-yl]cyclopropanecarboxamide;
cis-N-[5-[5-[(4-hydroxycyclohexyl)methoxy]-2-methyl-4-pyridyl]pyrazolo[1,5-a]pyridin-2-yl]cyclopropanecarboxamide;
N-[5-[5-[(2S)-2-amino-3,3-dimethyl-butoxy]-2-methyl-4-pyridyl]pyrazolo[1,5-a]pyridin-2-yl]cyclopropanecarboxamide;
N-[5-[5-[(2R)-2-amino-3,3-dimethyl-butoxy]-2-methyl-4-pyridyl]pyrazolo[1,5-a]pyridin-2-yl]cyclopropanecarboxamide;
trans-N-(5-(5-(((1r,4r)-4-hydroxy-4-methylcyclohexyl)oxy)-2-methylpyridin-4-yl)pyrazolo[1,5-a]pyridin-2-yl)cyclopropanecarboxamide;
N-[5-[5-[(1R,3S)-3-aminocyclopentoxy]-2-methyl-4-pyridyl]pyrazolo[1,5-a]pyridin-2-yl]cyclopropanecarboxamide;
N-[5-[5-[(1R,3R)-3-aminocyclopentoxy]-2-methyl-4-pyridyl]pyrazolo[1,5-a]pyridin-2-yl]cyclopropanecarboxamide;
N-[5-[5-[(1-aminocyclobutyl)methoxy]-2-methyl-4-pyridyl]pyrazolo[1,5-a]pyridin-2-yl]cyclopropanecarboxamide;
N-[5-[5-[(1-aminocyclopentyl)methoxy]-2-methyl-4-pyridyl]pyrazolo[1,5-a]pyridin-2-yl]cyclopropanecarboxamide;
N-[5-[2-methyl-5-[[1-(methylamino)cyclobutyl]methoxy]-4-pyridyl]pyrazolo[1,5-a]pyridin-2-yl]cyclopropanecarboxamide;
N-[5-[5-[[1-(cyclopropylamino)cyclobutyl]methoxy]-2-methyl-4-pyridyl]pyrazolo[1,5-a]pyridin-2-yl]cyclopropanecarboxamide;
trans-N-[5-[5-(4-hydroxycyclohexoxy)-2-methyl-4-pyridyl]pyrazolo[1,5-a]pyridin-2-yl]cyclopropanecarboxamide;
(*R)—N-[5-[5-(2-amino-3,3,3-trifluoro-2-methyl-propoxy)-2-methyl-4-pyridyl]pyrazolo[1,5-a]pyridin-2-yl]cyclopropanecarboxamide;
(*S)—N-[5-[5-(2-amino-3,3,3-trifluoro-2-methyl-propoxy)-2-methyl-4-pyridyl]pyrazolo[1,5-a]pyridin-2-yl]cyclopropanecarboxamide;
7-endo-N-(5-(5-(((1R,5S,7s)-9-ethyl-3-oxa-9-azabicyclo[3.3.1]nonan-7-yl)oxy)-2-methylpyridin-4-yl)pyrazolo[1,5-a]pyridin-2-yl)cyclopropanecarboxamide;
5-[5-[(1-amino-3,3-difluoro-cyclobutyl)methoxy]-2-methyl-4-pyridyl]-N-(1-methylpyrazol-3-yl)pyrazolo[1,5-a]pyridin-2-amine;
(1r,4r)-1-methyl-4-((6-methyl-4-(2-(1-methyl-1H-pyrazol-4-yl)amino)pyrazolo[1,5-a]pyridin-5-yl)pyridin-3-yl)oxy)cyclohexan-1-ol;
N-(5-(2-methyl-5-(((1S,4S,7R)-5-methyl-2-oxa-5-azabicyclo[2.2.1]heptan-7-yl)oxy)pyridin-4-yl)pyrazolo[1,5-a]pyridin-2-yl)cyclopropanecarboxamide;
N-(5-(5-(((1S,4S,7R)-2-oxa-5-azabicyclo[2.2.1]heptan-7-yl)oxy)-2-methylpyridin-4-yl)pyrazolo[1,5-a]pyridin-2-yl)cyclopropanecarboxamide;
N-[5-[2-methyl-5-[[(2R)-morpholin-2-yl]methoxy]-4-pyridyl]pyrazolo[1,5-a]pyridin-2-yl]cyclopropanecarboxamide;
N-[5-[5-[[(2S)-5,5-dimethylmorpholin-2-yl]methoxy]-2-methyl-4-pyridyl]pyrazolo[1,5-a]pyridin-2-yl]cyclopropanecarboxamide;
N-[5-[2-methyl-5-[[(2S,5R)-5-methylmorpholin-2-yl]methoxy]-4-pyridyl]pyrazolo[1,5-a]pyridin-2-yl]cyclopropanecarboxamide;
N-[5-[5-[(3-fluoro-1-methyl-azetidin-3-yl)methoxy]-2-methyl-4-pyridyl]pyrazolo[1,5-a]pyridin-2-yl]cyclopropanecarboxamide;
trans-N-[5-[5-(3-aminocyclobutoxy)-2-methyl-4-pyridyl]pyrazolo[1,5-a]pyridin-2-yl]cyclopropanecarboxamide;
cis-N-[5-[5-[(3-aminocyclobutyl)methoxy]-2-methyl-4-pyridyl]pyrazolo[1,5-a]pyridin-2-yl]cyclopropanecarboxamide;
2-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-N-methylisonicotinamide;
6-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-N-methylnicotinamide;
N-(1-methyl-3-(trifluoromethyl)-1H-pyrazol-5-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
N-(5-(5-(((1s,3s)-3-aminocyclobutyl)methoxy)-2-cyanopyridin-4-yl)pyrazolo[1,5-a]pyridin-2-yl)cyclopropanecarboxamide;
cis-N-[5-[2-cyano-5-[[3-(2-pyridylamino)cyclobutyl]methoxy]-4-pyridyl]pyrazolo[1,5-a]pyridin-2-yl]cyclopropanecarboxamide;
cis-N-[5-[2-cyano-5-[[3-[(1-methylpyrazol-3-yl)amino]cyclobutyl]methoxy]-4-pyridyl]pyrazolo[1,5-a]pyridin-2-yl]cyclopropanecarboxamide;
rac-N-[5-[2-cyano-5-[[4-hydroxy-4-(trifluoromethyl)cyclohexyl]methoxy]-4-pyridyl]pyrazolo[1,5-a]pyridin-2-yl]cyclopropanecarboxamide;
cis-N-(5-(5-(((1*S,3*R)-3-hydroxycyclopentyl)oxy)-2-methylpyridin-4-yl)pyrazolo[1,5-a]pyridin-2-yl)cyclopropanecarboxamide;
cis-N-(5-(5-(((1*R,3*S)-3-hydroxycyclopentyl)oxy)-2-methylpyridin-4-yl)pyrazolo[1,5-a]pyridin-2-yl)cyclopropanecarboxamide;
trans-4-[[6-methyl-4-[2-[(1-methylpyrazol-3-yl)amino]pyrazolo[1,5-a]pyridin-5-yl]-3-pyridyl]oxy]cyclohexanol;
trans-4-[[6-methyl-4-[2-(2-pyridylamino)pyrazolo[1,5-a]pyridin-5-yl]-3-pyridyl]oxy]cyclohexanol;
trans-N-(5-(5-(((1*S,3*S)-3-hydroxycyclopentyl)oxy)-2-methylpyridin-4-yl)pyrazolo[1,5-a]pyridin-2-yl)cyclopropanecarboxamide;
cis-4-[[6-methyl-4-[2-[(1-methylpyrazol-3-yl)amino]pyrazolo[1,5-a]pyridin-5-yl]-3-pyridyl]oxy]cyclohexanol;
trans-N-[5-[5-(4-hydroxycyclohexoxy)-2-prop-1-ynyl-4-pyridyl]pyrazolo[1,5-a]pyridin-2-yl]cyclopropanecarboxamide;
N-[5-[2-methyl-5-(4-oxocyclohexoxy)-4-pyridyl]pyrazolo[1,5-a]pyridin-2-yl]cyclopropanecarboxamide;
trans-4-[[4-[2-[(1-methylpyrazol-3-yl)amino]pyrazolo[1,5-a]pyridin-5-yl]-6-prop-1-ynyl-3-pyridyl]oxy]cyclohexanol;
rac-N-[5-[5-[(4,4-difluoro-1-hydroxy-cyclohexyl)methoxy]-2-methyl-4-pyridyl]pyrazolo[1,5-a]pyridin-2-yl]cyclopropanecarboxamide;
(*R)—N-(5-(2-methyl-5-(3,3,3-trifluoro-2-hydroxy-2-methylpropoxy)pyridin-4-yl)pyrazolo[1,5-a]pyridin-2-yl)cyclopropanecarboxamide;
(*S)—N-(5-(2-methyl-5-(3,3,3-trifluoro-2-hydroxy-2-methylpropoxy)pyridin-4-yl)pyrazolo[1,5-a]pyridin-2-yl)cyclopropanecarboxamide;
N-(5-(5-(((1r,4r)-4-hydroxy-4-(trifluoromethyl)cyclohexyl)oxy)-2-methylpyridin-4-yl)pyrazolo[1,5-a]pyridin-2-yl)cyclopropanecarboxamide;
N-(5-(5-(((1r,1r)-4-hydroxy-4-phenylcyclohexyl)oxy)-2-methylpyridin-4-yl)pyrazolo[1,5-a]pyridin-2-yl)cyclopropanecarboxamide;
(*R)—N-(5-(5-(2-cyclopropyl-2-hydroxypropoxy)-2-methylpyridin-4-yl)pyrazolo[1,5-a]pyridin-2-yl)cyclopropanecarboxamide;
trans-N-(5-(5-(((1*R,3*R)-3-hydroxycyclohexyl)oxy)-2-methylpyridin-4-yl)pyrazolo[1,5-a]pyridin-2-yl)cyclopropanecarboxamide;
trans-N-(5-(5-(((1*S,3*S)-3-hydroxycyclohexyl)oxy)-2-methylpyridin-4-yl)pyrazolo[1,5-a]pyridin-2-yl)cyclopropanecarboxamide;
N-(5-(5-((4-hydroxy-4-isopropylcyclohexyl)oxy)-2-methylpyridin-4-yl)pyrazolo[1,5-a]pyridin-2-yl)cyclopropanecarboxamide;
(*S)—N-(5-(5-(2-cyclohexyl-2-hydroxyethoxy)-2-methylpyridin-4-yl)pyrazolo[1,5-a]pyridin-2-yl)cyclopropanecarboxamide;
trans-4-[[4-[2-[(2,6-dimethylpyrimidin-4-yl)amino]pyrazolo[1,5-a]pyridin-5-yl]-6-methyl-3-pyridyl]oxy]cyclohexanol;
N-[5-[5-(4-hydroxyiminocyclohexoxy)-2-methyl-4-pyridyl]pyrazolo[1,5-a]pyridin-2-yl]cyclopropanecarboxamide;
1-[[4-[2-[(2,6-dimethylpyrimidin-4-yl)amino]pyrazolo[1,5-a]pyridin-5-yl]-6-methyl-3-pyridyl]oxy]-2-methyl-propan-2-ol;
1-[[4-[2-[(5,6-dimethylpyridazin-3-yl)amino]pyrazolo[1,5-a]pyridin-5-yl]-6-methyl-3-pyridyl]oxy]-2-methyl-propan-2-ol;
1-[[4-[2-[(2-ethyl-6-methyl-pyrimidin-4-yl)amino]pyrazolo[1,5-a]pyridin-5-yl]-6-methyl-3-pyridyl]oxy]-2-methyl-propan-2-ol;
(*R)-3-(((4-(2-((2,6-dimethylpyrimidin-4-yl)amino)pyrazolo[1,5-a]pyridin-5-yl)-6-methylpyridin-3-yl)oxy)methyl)tetrahydrofuran-3-ol;
(3R,5R)-5-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol;
1-[[4-[2-[[2-(methoxymethyl)-6-methyl-pyrimidin-4-yl]amino]pyrazolo[1,5-a]pyridin-5-yl]-6-methyl-3-pyridyl]oxy]-2-methyl-propan-2-ol;
N-(2,6-dimethylpyrimidin-4-yl)-5-(2-methyl-5-(((2*S,3*R)-2-methyltetrahydrofuran-3-yl)methoxy)pyridin-4-yl)pyrazolo[1,5-a]pyridin-2-amine;
N-(2,6-dimethylpyrimidin-4-yl)-5-(2-methyl-5-(((2*R,3*S)-2-methyltetrahydrofuran-3-yl)methoxy)pyridin-4-yl)pyrazolo[1,5-a]pyridin-2-amine;
3-[[5-[5-(2-hydroxy-2-methyl-propoxy)-2-methyl-4-pyridyl]pyrazolo[1,5-a]pyridin-2-yl]amino]-5-methyl-1H-pyridazin-6-one;
6-[[5-[5-(2-hydroxy-2-methyl-propoxy)-2-methyl-4-pyridyl]pyrazolo[1,5-a]pyridin-2-yl]amino]-2,4-dimethyl-pyridazin-3-one;
(*S)—N-[5-[5-[(3-cyanopyrrolidin-3-yl)methoxy]-2-methyl-4-pyridyl]pyrazolo[1,5-a]pyridin-2-yl]cyclopropanecarboxamide;
(*R)—N-[5-[5-[[(3R)-3-cyanopyrrolidin-3-yl]methoxy]-2-methyl-4-pyridyl]pyrazolo[1,5-a]pyridin-2-yl]cyclopropanecarboxamide;
(*S)—N-[5-[5-[(3-cyano-1-methyl-pyrrolidin-3-yl)methoxy]-2-methyl-4-pyridyl]pyrazolo[1,5-a]pyridin-2-yl]cyclopropanecarboxamide;
(*R)—N-[5-[5-[(3-cyano-1-methyl-pyrrolidin-3-yl)methoxy]-2-methyl-4-pyridyl]pyrazolo[1,5-a]pyridin-2-yl]cyclopropanecarboxamide;
(*R)—N-[5-[5-[(3-methoxypyrrolidin-3-yl)methoxy]-2-methyl-4-pyridyl]pyrazolo[1,5-a]pyridin-2-yl]cyclopropanecarboxamide;
(*S)—N-[5-[5-[(3-methoxypyrrolidin-3-yl)methoxy]-2-methyl-4-pyridyl]pyrazolo[1,5-a]pyridin-2-yl]cyclopropanecarboxamide;
(*R)-3-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol;
1-[[4-[2-[(2,6-dimethylpyrimidin-4-yl)amino]pyrazolo[1,5-a]pyridin-5-yl]-6-methyl-3-pyridyl]oxymethyl]cyclobutanol;
(2R,3S)-2-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol;
(*S)—N-(2,6-dimethylpyrimidin-4-yl)-5-[2-methyl-5-(8-oxaspiro[4.4]nonan-7-ylmethoxy)-4-pyridyl]pyrazolo[1,5-a]pyridin-2-amine;
(*S)-5-(5-((6-oxaspiro[3.4]octan-7-yl)methoxy)-2-methylpyridin-4-yl)-N-(2,6-dimethylpyrimidin-4-yl)pyrazolo[1,5-a]pyridin-2-amine;
N-[5-[5-[(3-cyanoazetidin-3-yl)methoxy]-2-methyl-4-pyridyl]pyrazolo[1,5-a]pyridin-2-yl]cyclopropanecarboxamide;
(*S)—N-[5-[5-[(3-hydroxypyrrolidin-3-yl)methoxy]-2-methyl-4-pyridyl]pyrazolo[1,5-a]pyridin-2-yl]cyclopropanecarboxamide;
(*R)—N-[5-[5-[(3-hydroxypyrrolidin-3-yl)methoxy]-2-methyl-4-pyridyl]pyrazolo[1,5-a]pyridin-2-yl]cyclopropanecarboxamide;
(1r,4r)-4-((4-(2-((2-ethyl-6-methylpyrimidin-4-yl)amino)pyrazolo[1,5-a]pyridin-5-yl)-6-methylpyridin-3-yl)oxy)cyclohexan-1-ol;
(1r,4r)-4-((4-(2-((2-(methoxymethyl)-6-methylpyrimidin-4-yl)amino)pyrazolo[1,5-a]pyridin-5-yl)-6-methylpyridin-3-yl)oxy)cyclohexan-1-ol;
6-((5-(5-(((1r,4r)-4-hydroxycyclohexyl)oxy)-2-methylpyridin-4-yl)pyrazolo[1,5-a]pyridin-2-yl)amino)-2,4-dimethylpyridazin-3(2H)-one;
(1r,4r)-4-((6-methyl-4-(2-((6-methyl-2-(methylamino)pyrimidin-4-yl)amino)pyrazolo[1,5-a]pyridin-5-yl)pyridin-3-yl)oxy)cyclohexan-1-ol;
2-methyl-1-[[6-methyl-4-[2-[[6-methyl-2-(methylamino)pyrimidin-4-yl]amino]pyrazolo[1,5-a]pyridin-5-yl]-3-pyridyl]oxy]propan-2-ol;
1-[[4-[2-[(2-cyclopropyl-6-methyl-pyrimidin-4-yl)amino]pyrazolo[1,5-a]pyridin-5-yl]-6-methyl-3-pyridyl]oxy]-2-methyl-propan-2-ol;
(*S)—N-(2,6-dimethylpyrimidin-4-yl)-5-[2-methyl-5-(9-oxaspiro[4.4]nonan-8-ylmethoxy)-4-pyridyl]pyrazolo[1,5-a]pyridin-2-amine;
(*R)—N-(2,6-dimethylpyrimidin-4-yl)-5-[2-methyl-5-(9-oxaspiro[4.4]nonan-8-ylmethoxy)-4-pyridyl]pyrazolo[1,5-a]pyridin-2-amine;
(*S)—N-(2,6-dimethylpyrimidin-4-yl)-5-[2-methyl-5-[(2-methyltetrahydrofuran-2-yl)methoxy]-4-pyridyl]pyrazolo[1,5-a]pyridin-2-amine
N-[5-[5-[(3-methoxyazetidin-3-yl)methoxy]-2-methyl-4-pyridyl]pyrazolo[1,5-a]pyridin-2-yl]cyclopropanecarboxamide;
(1R,2R)-2-methyl-N-[5-[2-methyl-5-[[(1R,4R)-5-oxa-2-azabicyclo[2.2.1]heptan-4-yl]methoxy]-4-pyridyl]pyrazolo[1,5-a]pyridin-2-yl]cyclopropanecarboxamide;
N-(6-cyclopropylpyridazin-3-yl)-5-[2-methyl-5-[[(1R,4R)-2-methyl-5-oxa-2-azabicyclo[2.2.1]heptan-4-yl]methoxy]-4-pyridyl]pyrazolo[1,5-a]pyridin-2-amine;
5-[2-methyl-5-[[(1R,4R)-2-methyl-5-oxa-2-azabicyclo[2.2.1]heptan-4-yl]methoxy]-4-pyridyl]-N-(5-methylpyrazin-2-yl)pyrazolo[1,5-a]pyridin-2-amine;
N-[5-[2-methyl-5-[[(1S,4S)-2-methyl-5-oxa-2-azabicyclo[2.2.1]heptan-4-yl]methoxy]-4-pyridyl]pyrazolo[1,5-a]pyridin-2-yl]cyclopropanecarboxamide;
N-(2,6-dimethylpyrimidin-4-yl)-5-[2-methyl-5-[[(1R,4R)-5-oxa-2-azabicyclo[2.2.1]heptan-4-yl]methoxy]-4-pyridyl]pyrazolo[1,5-a]pyridin-2-amine;
4-[[6-methyl-4-[2-(1H-pyrrolo[2,3-b]pyridin-6-ylamino)pyrazolo[1,5-a]pyridin-5-yl]-3-pyridyl]oxy]cyclohexanol;
2-methyl-1-[[6-methyl-4-[2-(1H-pyrrolo[2,3-b]pyridin-6-ylamino)pyrazolo[1,5-a]pyridin-5-yl]-3-pyridyl]oxy]propan-2-ol;
4-[[5-[5-(4-hydroxycyclohexoxy)-2-methyl-4-pyridyl]pyrazolo[1,5-a]pyridin-2-yl]amino]-2,6-dimethoxy-N-(2,2,2-trifluoroethyl)benzamide;
N-cyclopropyl-4-[[5-[5-(4-hydroxycyclohexoxy)-2-methyl-4-pyridyl]pyrazolo[1,5-a]pyridin-2-yl]amino]-2-methoxy-benzamide;
N-ethyl-4-[[5-[5-(4-hydroxycyclohexoxy)-2-methyl-4-pyridyl]pyrazolo[1,5-a]pyridin-2-yl]amino]-2,6-dimethoxy-benzamide;
6-[[5-[5-(2-hydroxy-2-methyl-propoxy)-2-methyl-4-pyridyl]pyrazolo[1,5-a]pyridin-2-yl]amino]-N,N,4-trimethyl-pyridine-2-carboxamide;
N-cyclopropyl-4-[[5-[5-(4-hydroxycyclohexoxy)-2-methyl-4-pyridyl]pyrazolo[1,5-a]pyridin-2-yl]amino]-2,6-dimethoxy-benzamide;
methyl 4-[[5-[5-(4-hydroxycyclohexoxy)-2-methyl-4-pyridyl]pyrazolo[1,5-a]pyridin-2-yl]amino]-2-methoxy-benzoate;
2-ethyl-6-[[5-[5-(2-hydroxy-2-methyl-propoxy)-2-methyl-4-pyridyl]pyrazolo[1,5-a]pyridin-2-yl]amino]-4-methyl-pyridazin-3-one;
N-[5-[2-methyl-5-[[(1R,4R)-5-oxa-2-azabicyclo[2.2.1]heptan-4-yl]methoxy]-4-pyridyl]pyrazolo[1,5-a]pyridin-2-yl]cyclopropanecarboxamide;
N-[5-[2-methyl-5-[[(1R,4R)-2-methyl-5-oxa-2-azabicyclo[2.2.1]heptan-4-yl]methoxy]-4-pyridyl]pyrazolo[1,5-a]pyridin-2-yl]cyclopropanecarboxamide;
(1R,2R)-2-methyl-N-[5-[2-methyl-5-[[(1R,4R)-2-methyl-5-oxa-2-azabicyclo[2.2.1]heptan-4-yl]methoxy]-4-pyridyl]pyrazolo[1,5-a]pyridin-2-yl]cyclopropanecarboxamide;
5-[2-methyl-5-[[(1S,4S)-5-oxa-2-azabicyclo[2.2.1]heptan-4-yl]methoxy]-4-pyridyl]-N-(5-methylpyrazin-2-yl)pyrazolo[1,5-a]pyridin-2-amine;
N-(2,6-dimethylpyrimidin-4-yl)-5-[5-[[(1R,4R)-5-oxa-2-azabicyclo[2.2.1]heptan-4-yl]methoxy]-2-(trifluoromethyl)-4-pyridyl]pyrazolo[1,5-a]pyridin-2-amine;
N-[5-[2-methyl-5-[[(1S,4S)-5-oxa-2-azabicyclo[2.2.1]heptan-4-yl]methoxy]-4-pyridyl]pyrazolo[1,5-a]pyridin-2-yl]cyclopropanecarboxamide;
N-(2,6-dimethylpyrimidin-4-yl)-5-[5-[[(1R,4R)-5-oxa-2-azabicyclo[2.2.1]heptan-4-yl]methoxy]-2-(trifluoromethyl)-4-pyridyl]pyrazolo[1,5-a]pyridin-2-amine;
N-(6-cyclopropylpyridazin-3-yl)-5-[2-methyl-5-[[(1R,4R)-5-oxa-2-azabicyclo[2.2.1]heptan-4-yl]methoxy]-4-pyridyl]pyrazolo[1,5-a]pyridin-2-amine;
5-[2-methyl-5-[[(1R,4R)-2-methyl-5-oxa-2-azabicyclo[2.2.1]heptan-4-yl]methoxy]-4-pyridyl]-N-(6-methylpyridazin-3-yl)pyrazolo[1,5-a]pyridin-2-amine;
5-[2-methyl-5-[[(1R,4R)-5-oxa-2-azabicyclo[2.2.1]heptan-4-yl]methoxy]-4-pyridyl]-N-(5-methylpyrazin-2-yl)pyrazolo[1,5-a]pyridin-2-amine;
(*R)—N-(2,6-dimethylpyrimidin-4-yl)-5-[2-methyl-5-(8-oxaspiro[4.4]nonan-7-ylmethoxy)-4-pyridyl]pyrazolo[1,5-a]pyridin-2-amine;
2-[[4-[2-[(2,6-dimethylpyrimidin-4-yl)amino]pyrazolo[1,5-a]pyridin-5-yl]-6-methyl-3-pyridyl]oxymethyl]-1,1,1,3,3,3-hexafluoro-propan-2-ol
1-[[4-[2-(imidazo[1,2-b]pyridazin-6-ylamino)pyrazolo[1,5-a]pyridin-5-yl]-6-methyl-3-pyridyl]oxy]-2-methyl-propan-2-ol;
1-[[4-[2-[(2-tert-butyl-6-methyl-pyrimidin-4-yl)amino]pyrazolo[1,5-a]pyridin-5-yl]-6-methyl-3-pyridyl]oxy]-2-methyl-propan-2-ol;
3-[[4-[2-[(2,6-dimethylpyrimidin-4-yl)amino]pyrazolo[1,5-a]pyridin-5-yl]-6-methyl-3-pyridyl]oxy]-2,2-dimethyl-propanenitrile;
(*S)-3-[[4-[2-[(2,6-dimethylpyrimidin-4-yl)amino]pyrazolo[1,5-a]pyridin-5-yl]-6-methyl-3-pyridyl]oxy]-1,1,1-trifluoro-2-methyl-propan-2-ol;
(*R)-3-[[4-[2-[(2,6-dimethylpyrimidin-4-yl)amino]pyrazolo[1,5-a]pyridin-5-yl]-6-methyl-3-pyridyl]oxy]-1,1,1-trifluoro-2-methyl-propan-2-ol;
2-(cyclopropylmethyl)-6-[[5-[5-(2-hydroxy-2-methyl-propoxy)-2-methyl-4-pyridyl]pyrazolo[1,5-a]pyridin-2-yl]amino]-4-methyl-pyridazin-3-one;
N-(2,6-dimethylpyrimidin-4-yl)-5-[2-methyl-5-(4-methylsulfonylcyclohexoxy)-4-pyridyl]pyrazolo[1,5-a]pyridin-2-amine;
(rac-)-5-[5-(2-amino-3,3,3-trifluoro-2-methyl-propoxy)-2-methyl-4-pyridyl]-N-(2,6-dimethylpyrimidin-4-yl)pyrazolo[1,5-a]pyridin-2-amine;
4-[[5-[5-(2-cyano-2-methyl-propoxy)-2-methyl-4-pyridyl]pyrazolo[1,5-a]pyridin-2-yl]amino]-N-cyclopropyl-2,6-dimethoxy-benzamide;
3-[[4-[2-[(1,5-dimethyl-6-oxo-pyridazin-3-yl)amino]pyrazolo[1,5-a]pyridin-5-yl]-6-methyl-3-pyridyl]oxy]-2,2-dimethyl-propanenitrile;
(*R)-5-[5-(2-Amino-3,3,3-trifluoro-2-methyl-propoxy)-2-methyl-4-pyridyl]-N-(2,6-dimethylpyrimidin-4-yl)pyrazolo[1,5-a]pyridin-2-amine;
N-(2,6-dimethylpyrimidin-4-yl)-5-[5-[(2-methoxyphenyl)methoxy]-2-methyl-4-pyridyl]pyrazolo[1,5-a]pyridin-2-amine;
3-[[4-[2-[(2,6-dimethylpyrimidin-4-yl)amino]pyrazolo[1,5-a]pyridin-5-yl]-6-methyl-3-pyridyl]oxymethyl]phenol;
N-(2,6-dimethylpyrimidin-4-yl)-5-[5-[(3-ethyloxetan-3-yl)methoxy]-2-methyl-4-pyridyl]pyrazolo[1,5-a]pyridin-2-amine;
[3-[[4-[2-[(2,6-dimethylpyrimidin-4-yl)amino]pyrazolo[1,5-a]pyridin-5-yl]-6-methyl-3-pyridyl]oxymethyl]oxetan-3-yl]methanol;
3-[[4-[2-[(2,6-dimethylpyrimidin-4-yl)amino]pyrazolo[1,5-a]pyridin-5-yl]-6-methyl-3-pyridyl]oxymethyl]oxetane-3-carbonitrile;
N-(2,6-dimethylpyrimidin-4-yl)-5-[2-methyl-5-[(3S)-pyrrolidin-3-yl]oxy-4-pyridyl]pyrazolo[1,5-a]pyridin-2-amine;
N-[5-[2-methyl-5-[(3S)-pyrrolidin-3-yl]oxy-4-pyridyl]pyrazolo[1,5-a]pyridin-2-yl]cyclopropanecarboxamide;
N-(2,6-dimethylpyrimidin-4-yl)-5-[5-[(3R,4S)-4-fluoropyrrolidin-3-yl]oxy-2-methyl-4-pyridyl]pyrazolo[1,5-a]pyridin-2-amine;
N-[5-[5-[(3R,4S)-4-fluoropyrrolidin-3-yl]oxy-2-methyl-4-pyridyl]pyrazolo[1,5-a]pyridin-2-yl]cyclopropanecarboxamide;
N-[5-[2-prop-1-ynyl-5-[(3S)-pyrrolidin-3-yl]oxy-4-pyridyl]pyrazolo[1,5-a]pyridin-2-yl]cyclopropanecarboxamide;
N-[5-[5-[(3R,4S)-4-fluoropyrrolidin-3-yl]oxy-2-prop-1-ynyl-4-pyridyl]pyrazolo[1,5-a]pyridin-2-yl]cyclopropanecarboxamide;
6-[[5-[5-(2-hydroxy-2-methyl-propoxy)-2-methyl-4-pyridyl]pyrazolo[1,5-a]pyridin-2-yl]amino]-2-isobutyl-4-methyl-pyridazin-3-one;
2-[2-(difluoromethoxy)ethyl]-6-[[5-[5-(4-hydroxycyclohexoxy)-2-methyl-4-pyridyl]pyrazolo[1,5-a]pyridin-2-yl]amino]-4-methyl-pyridazin-3-one;
2-[2-(difluoromethoxy)ethyl]-6-[[5-[5-(2-hydroxy-2-methyl-propoxy)-2-methyl-4-pyridyl]pyrazolo[1,5-a]pyridin-2-yl]amino]-4-methyl-pyridazin-3-one;
2-methyl-1-[[6-methyl-4-[2-[(6-methyl-2-pyrrolidin-1-yl-pyrimidin-4-yl)amino]pyrazolo[1,5-a]pyridin-5-yl]-3-pyridyl]oxy]propan-2-ol;
2-methyl-1-[[6-methyl-4-[2-[(6-methyl-2-morpholino-pyrimidin-4-yl)amino]pyrazolo[1,5-a]pyridin-5-yl]-3-pyridyl]oxy]propan-2-ol;
4-[[6-methyl-4-[2-[(6-methyl-2-pyrrolidin-1-yl-pyrimidin-4-yl)amino]pyrazolo[1,5-a]pyridin-5-yl]-3-pyridyl]oxy]cyclohexanol;
4-[[6-methyl-4-[2-[(6-methyl-2-morpholino-pyrimidin-4-yl)amino]pyrazolo[1,5-a]pyridin-5-yl]-3-pyridyl]oxy]cyclohexanol;
4-[[6-methyl-4-[2-(5H-pyrrolo[2,3-b]pyrazin-3-ylamino)pyrazolo[1,5-a]pyridin-5-yl]-3-pyridyl]oxy]cyclohexanol;
(1r,4r)-4-((6-methyl-4-(2-(pyridin-3-ylamino)pyrazolo[1,5-a]pyridin-5-yl)pyridin-3-yl)oxy)cyclohexan-1-ol;
2-methyl-1-[[6-methyl-4-[2-(5H-pyrrolo[2,3-b]pyrazin-3-ylamino)pyrazolo[1,5-a]pyridin-5-yl]-3-pyridyl]oxy]propan-2-ol;
2-methyl-1-[[6-methyl-4-[2-[(1-methylpyrazolo[3,4-b]pyridin-3-yl)amino]pyrazolo[1,5-a]pyridin-5-yl]-3-pyridyl]oxy]propan-2-ol;
2-ethyl-6-[[5-[5-[[(2R,3S)-3-hydroxytetrahydrofuran-2-yl]methoxy]-2-methyl-4-pyridyl]pyrazolo[1,5-a]pyridin-2-yl]amino]-4-methyl-pyridazin-3-one;
(2R,3S)-2-[[6-methyl-4-[2-[(6-methyl-2-morpholino-pyrimidin-4-yl)amino]pyrazolo[1,5-a]pyridin-5-yl]-3-pyridyl]oxymethyl]tetrahydrofuran-3-ol;
(2R,3S)-2-[[4-[2-[[5-(difluoromethyl)pyrazin-2-yl]amino]pyrazolo[1,5-a]pyridin-5-yl]-6-methyl-3-pyridyl]oxymethyl]tetrahydrofuran-3-ol;
2-[2-(difluoromethoxy)ethyl]-6-[[5-[5-[[(2R,3S)-3-hydroxytetrahydrofuran-2-yl]methoxy]-2-methyl-4-pyridyl]pyrazolo[1,5-a]pyridin-2-yl]amino]-4-methyl-pyridazin-3-one;
(2R,3S)-2-[[6-methyl-4-[2-[(6-methylpyrazin-2-yl)amino]pyrazolo[1,5-a]pyridin-5-yl]-3-pyridyl]oxymethyl]tetrahydrofuran-3-ol;
(2R,3S)-2-[[4-[2-[(2-tert-butyl-6-methyl-pyrimidin-4-yl)amino]pyrazolo[1,5-a]pyridin-5-yl]-6-methyl-3-pyridyl]oxymethyl]tetrahydrofuran-3-ol;
(2R,3S)-2-[[6-methyl-4-[2-[(1-methylindazol-3-yl)amino]pyrazolo[1,5-a]pyridin-5-yl]-3-pyridyl]oxymethyl]tetrahydrofuran-3-ol;
(2R,3S)-2-[[4-[2-[(1-methylpyrazol-4-yl)amino]pyrazolo[1,5-a]pyridin-5-yl]-6-prop-1-ynyl-3-pyridyl]oxymethyl]tetrahydrofuran-3-ol;
(2R,3S)-2-[[4-[2-[(1-methylpyrazol-3-yl)amino]pyrazolo[1,5-a]pyridin-5-yl]-6-prop-1-ynyl-3-pyridyl]oxymethyl]tetrahydrofuran-3-ol;
N-(2,6-dimethylpyrimidin-4-yl)-5-[5-[[(1S,4S)-5-oxa-2-azabicyclo[2.2.1]heptan-4-yl]methoxy]-2-prop-1-ynyl-4-pyridyl]pyrazolo[1,5-a]pyridin-2-amine;
5-[2-chloro-5-[[(1S,4S)-5-oxa-2-azabicyclo[2.2.1]heptan-4-yl]methoxy]-4-pyridyl]-N-(2,6-dimethylpyrimidin-4-yl)pyrazolo[1,5-a]pyridin-2-amine;
(*S)-5-[5-(2-amino-3,3,3-trifluoro-2-methyl-propoxy)-2-methyl-4-pyridyl]-N-(2,6-dimethylpyrimidin-4-yl)pyrazolo[1,5-a]pyridin-2-amine;
2,4-dimethyl-6-((5-(2-methyl-5-(41s,4s)-4-(methyl sulfonyl)cyclohexyl)oxy)pyridin-4-yl)pyrazolo[1,5-a]pyridin-2-yl)amino)pyridazin-3(2H)-one;
2,4-dimethyl-6-((5-(2-methyl-5-(((1r,4r)-4-(methyl sulfonyl)cyclohexyl)oxy)pyridin-4-yl)pyrazolo[1,5-a]pyridin-2-yl)amino)pyridazin-3(2H)-one;
2,2-dimethyl-3[[6-methyl-4-[2-(1H-pyrrolo[2,3-b]pyridin-6-ylamino)pyrazolo[1,5-a]pyridin-5-yl]-3-pyridyl]oxy]propanenitrile;
3-[[4-[2-(imidazo[1,2-b]pyridazin-6-ylamino)pyrazolo[1,5-a]pyridin-5-yl]-6-methyl-3-pyridyl]oxy]-2,2-dimethyl-propanenitrile;
2,2-dimethyl-3-[[6-methyl-4-[2-[[6-methyl-2-(methylamino)pyrimidin-4-yl]amino]pyrazolo[1,5-a]pyridin-5-yl]-3-pyridyl]oxy]propanenitrile;
3-[[4-[2-[(2-cyclopropyl-6-methyl-pyrimidin-4-yl)amino]pyrazolo[1,5-a]pyridin-5-yl]-6-methyl-3-pyridyl]oxy]-2,2-dimethyl-propanenitrile;
(*R)-6-[[5-[5-(2-amino-3,3,3-trifluoro-2-methyl-propoxy)-2-methyl-4-pyridyl]pyrazolo[1,5-a]pyridin-2-yl]amino]-2,4-dimethyl-pyridazin-3-one;
(*S)-6-[[5-[5-(2-amino-3,3,3-trifluoro-2-methyl-propoxy)-2-methyl-4-pyridyl]pyrazolo[1,5-a]pyridin-2-yl]amino]-2,4-dimethyl-pyridazin-3-one;
1-[[4-[2-[(2,6-dimethylpyrimidin-4-yl)amino]pyrazolo[1,5-a]pyridin-5-yl]-6-methyl-3-pyridyl]oxymethyl]cyclopropanecarboxamide;
5-[5-(3-aminocyclobutoxy)-2-methyl-4-pyridyl]-N-(2,6-dimethylpyrimidin-4-yl)pyrazolo[1,5-a]pyridin-2-amine;
4-[[4-[2-[(2,6-dimethylpyrimidin-4-yl)amino]pyrazolo[1,5-a]pyridin-5-yl]-6-methyl-3-pyridyl]oxy]-2,2-dimethyl-butanenitrile;
3-[[4-[2-[(1-cyclopropyl-5-methyl-6-oxo-pyridazin-3-yl)amino]pyrazolo[1,5-a]pyridin-5-yl]-6-methyl-3-pyridyl]oxy]-2,2-dimethyl-propanenitrile;
2-cyclopropyl-6-[[5-[5-[[(2R,3S)-3-hydroxytetrahydrofuran-2-yl]methoxy]-2-methyl-4-pyridyl]pyrazolo[1,5-a]pyridin-2-yl]amino]-4-methyl-pyridazin-3-one;
2-cyclopropyl-6-[[5-[5-(4-hydroxycyclohexoxy)-2-methyl-4-pyridyl]pyrazolo[1,5-a]pyridin-2-yl]amino]-4-methyl-pyridazin-3-one;
N-[5-[2-methyl-5-[[(1S,5R)-9-methyl-3-oxa-9-azabicyclo[3.3.1]nonan-7-yl]oxy]-4-pyridyl]pyrazolo[1,5-c]pyrimidin-2-yl]cyclopropanecarboxamide;
6-[2-methyl-5-[[(1S,5R)-3-oxa-9-azabicyclo[3.3.1]nonan-7-yl]oxy]-4-pyridyl]-N-(5-methylpyrazin-2-yl)imidazo[1,2-a]pyrazin-2-amine;
6-[2-methyl-5-[[(1S,5R)-3-oxa-9-azabicyclo[3.3.1]nonan-7-yl]oxy]-4-pyridyl]-N-(6-methylpyrazin-2-yl)imidazo[1,2-a]pyrazin-2-amine;
6-[2-methyl-5-[[(1S,5R)-3-oxa-9-azabicyclo[3.3.1]nonan-7-yl]oxy]-4-pyridyl]-N-(1-methylpyrazol-4-yl)imidazo[1,2-a]pyrazin-2-amine;
N-cyclopropyl-4-[[5-[5-(4-hydroxycyclohexoxy)-2-methyl-4-pyridyl]pyrazolo[1,5-a]pyridin-2-yl]amino]benzenesulfonamide;
4-[[5-[5-(4-hydroxycyclohexoxy)-2-methyl-4-pyridyl]pyrazolo[1,5-a]pyridin-2-yl]amino]-N,N-dimethyl-benzenesulfonamide;
rac-4-[[4-[2-[(2,6-dimethylpyrimidin-4-yl)amino]pyrazolo[1,5-a]pyridin-5-yl]-6-methyl-3-pyridyl]oxymethyl]-1,5,5-trimethyl-pyrrolidin-2-one;
N-cyclopropyl-4-[[5-[5-(2-hydroxy-2-methyl-propoxy)-2-methyl-4-pyridyl]pyrazolo[1,5-a]pyridin-2-yl]amino]-2,6-dimethyl-benzamide;
1-[[4-[2-[(2,6-dimethylpyrimidin-4-yl)amino]pyrazolo[1,5-a]pyridin-5-yl]-6-(fluoromethyl)-3-pyridyl]oxy]-2-methyl-propan-2-ol;
3-[[4-[2-[(2,6-dimethylpyrimidin-4-yl)amino]pyrazolo[1,5-a]pyridin-5-yl]-6-ethyl-3-pyridyl]oxy]-2,2-dimethyl-propanenitrile;
3-[[6-(difluoromethoxy)-4-[2-[(2,6-dimethylpyrimidin-4-yl)amino]pyrazolo[1,5-a]pyridin-5-yl]-3-pyridyl]oxy]-2,2-dimethyl-propanenitrile;
4-[[5-[5-(4-hydroxycyclohexoxy)-2-methyl-4-pyridyl]pyrazolo[1,5-a]pyridin-2-yl]amino]-N-isopropyl-2,6-dimethoxy-benzamide;
4-[[5-[5-(4-hydroxycyclohexoxy)-2-methyl-4-pyridyl]pyrazolo[1,5-a]pyridin-2-yl]amino]-2,6-dimethoxy-N-methyl-benzamide;
4-[[5-[5-(4-hydroxycyclohexoxy)-2-methyl-4-pyridyl]pyrazolo[1,5-a]pyridin-2-yl]amino]-2,6-dimethoxy-N,N-dimethyl-benzamide;
N-cyclopropyl-2-(difluoromethoxy)-4-[[5-[5-(4-hydroxycyclohexoxy)-2-methyl-4-pyridyl]pyrazolo[1,5-a]pyridin-2-yl]amino]-6-methoxy-benzamide;
N-cyclopropyl-6-[[5-[5-(4-hydroxycyclohexoxy)-2-methyl-4-pyridyl]pyrazolo[1,5-a]pyridin-2-yl]amino]-2,4-dimethyl-pyridine-3-carboxamide;
N-cyclopropyl-6-[[5-[5-(2-hydroxy-2-methyl-propoxy)-2-methyl-4-pyridyl]pyrazolo[1,5-a]pyridin-2-yl]amino]-2,4-dimethyl-pyridine-3-carboxamide;
(1S,5R)-7-[[4-[2-[(2,6-dimethylpyrimidin-4-yl)amino]pyrazolo[1,5-a]pyridin-5-yl]-6-methyl-3-pyridyl]oxy]-3-oxa-9-azabicyclo[3.3.1]nonane-9-carbonitrile;
2,4-dimethyl-6-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]pyridazin-3-one;
N-(3-fluoro-4-methyl-2-pyridyl)-5-[2-methyl-5-[[(1S,5R)-3-oxa-9-azabicyclo[3.3.1]nonan-7-yl]oxy]-4-pyridyl]pyrazolo[1,5-a]pyridin-2-amine;
N-[5-[5-[[(1S,5R)-9-acetyl-3-oxa-9-azabicyclo[3.3.1]nonan-7-yl]oxy]-2-methyl-4-pyridyl]pyrazolo[1,5-a]pyridin-2-yl]cyclopropanecarboxamide;
N-isopropyl-2,6-dimethoxy-4-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]benzamide;
N-isopropyl-2-methyl-5-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]pyrazole-3-carboxamide
(4R)-4-[[4-[2-[(2,6-dimethylpyrimidin-4-yl)amino]pyrazolo[1,5-a]pyridin-5-yl]-6-methyl-3-pyridyl]oxymethyl]oxazolidin-2-one;
4-[[4-[2-[(2,6-dimethylpyrimidin-4-yl)amino]pyrazolo[1,5-a]pyridin-5-yl]-6-methyl-3-pyridyl]oxy]cyclohexanecarboxamide;
N-(2,6-dimethylpyrimidin-4-yl)-5-[5-[(3R)-1,1-dioxothiolan-3-yl]oxy-2-methyl-4-pyridyl]pyrazolo[1,5-a]pyridin-2-amine;
4-[[5-[5-(2-hydroxy-2-methyl-propoxy)-2-methyl-4-pyridyl]pyrazolo[1,5-a]pyridin-2-yl]amino]-N-isopropyl-2-methyl-benzamide;
(3*R,6*S)-6-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol;
(3*S,6*S)-6-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol;
N-(2,6-dimethylpyrimidin-4-yl)-6-[2-methyl-5-[[(1R,5S)-3-oxa-9-azabicyclo[3.3.1]nonan-7-yl]oxy]-4-pyridyl]imidazo[1,2-a]pyrazin-2-amine;
N-(2,6-dimethylpyrimidin-4-yl)-6-[2-methyl-5-[[(1R,5S)-9-methyl-3-oxa-9-azabicyclo[3.3.1]nonan-7-yl]oxy]-4-pyridyl]imidazo[1,2-a]pyrazin-2-amine;
(3*R, 5*S)-5-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol;
(RS)—N-(2,6-dimethylpyrimidin-4-yl)-5-[5-[(1,1-dioxo-1,2,5-thiadiazolidin-3-yl)methoxy]-2-methyl-4-pyridyl]pyrazolo[1,5-a]pyridin-2-amine;
(RS)—N-(2,6-dimethylpyrimidin-4-yl)-5-[5-[(1,1-dioxo-1,2-thiazolidin-3-yl)methoxy]-2-methyl-4-pyridyl]pyrazolo[1,5-a]pyridin-2-amine;
(*R)—N-(2,6-dimethylpyrimidin-4-yl)-5-[2-methyl-5-(2-methyl-1,1-dioxo-thiazinan-4-yl)oxy-4-pyridyl]pyrazolo[1,5-a]pyridin-2-amine;
(*S)—N-(2,6-dimethylpyrimidin-4-yl)-5-[2-methyl-5-(2-methyl-1,1-dioxo-thiazinan-4-yl)oxy-4-pyridyl]pyrazolo[1,5-a]pyridin-2-amine;
6-[[5-[5-[[(1S,5R)-9-acetyl-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide;
2,6-dimethoxy-N-methyl-4-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]benzamide;
6-[[5-[5-[[(1R,5S,7s)-9-acetyl-3-oxa-9-azabicyclo[3.3.1]nonan-7-yl]oxy]-2-methyl-4-pyridyl]pyrazolo[1,5-a]pyridin-2-yl]amino]-N,2,4-trimethyl-pyridine-3-carboxamide;
5-[5-(2-amino-2-methyl-propoxy)-2-methyl-4-pyridyl]-N-(2,6-dimethylpyrimidin-4-yl)pyrazolo[1,5-a]pyridin-2-amine;
N-[2-[[4-[2-[(2,6-dimethylpyrimidin-4-yl)amino]pyrazolo[1,5-a]pyridin-5-yl]-6-methyl-3-pyridyl]oxy]-1,1-dimethyl-ethyl]acetamide;
N-[2-[[4-[2-[(2,6-dimethylpyrimidin-4-yl)amino]pyrazolo[1,5-a]pyridin-5-yl]-6-methyl-3-pyridyl]oxy]-1,1-dimethyl-ethyl]methanesulfonamide;
4-[[4-[2-[(2,6-dimethylpyrimidin-4-yl)amino]pyrazolo[1,5-a]pyridin-5-yl]-6-prop-1-ynyl-3-pyridyl]amino]cyclohexanol;
(*R)—N-(2,6-dimethylpyrimidin-4-yl)-5-[5-[(1,1-dioxothiolan-3-yl)methoxy]-2-methyl-4-pyridyl]pyrazolo[1,5-a]pyridin-2-amine;
(*S)—N-(2,6-dimethylpyrimidin-4-yl)-5-[5-[(1,1-dioxothiolan-3-yl)methoxy]-2-methyl-4-pyridyl]pyrazolo[1,5-a]pyridin-2-amine;
(4S)-4-[[4-[2-[(2,6-dimethylpyrimidin-4-yl)amino]pyrazolo[1,5-a]pyridin-5-yl]-6-methyl-3-pyridyl]oxymethyl]-1-methyl-imidazolidin-2-one;
5-[2-methyl-5-[[(1S,5R, 7s)-3-oxa-9-azabicyclo[3.3.1]nonan-7-yl]oxy]-4-pyridyl]-N-[6-(trifluoromethyl)-2-pyridyl]pyrazolo[1,5-a]pyridin-2-amine;
N-methyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-2-carboxamide;
5-[2-methyl-5-[[(1S,5R,7s)-3-oxa-9-azabicyclo[3.3.1]nonan-7-yl]oxy]-4-pyridyl]-N-pyridazin-4-yl-pyrazolo[1,5-a]pyridin-2-amine;
N-(2-hydroxyethyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxylic acid;
N-(2-hydroxy-2-methyl-propyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
5-[2-methyl-5-[[(1S,5R,7s)-3-oxa-9-azabicyclo[3.3.1]nonan-7-yl]oxy]-4-pyridyl]-N-(2-methylpyrimidin-4-yl)pyrazolo[1,5-a]pyridin-2-amine;
6-[[5-[2-methyl-5-[[(1R,5S,7s)-3-oxa-9-azabicyclo[3.3.1]nonan-7-yl]oxy]-4-pyridyl]pyrazolo[1,5-a]pyridin-2-yl]amino]-N-(tetrahydropyran-4-ylmethyl)pyridine-3-carboxamide;
5-[2-methyl-5-[[(1S,5R,7s)-3-oxa-9-azabicyclo[3.3.1]nonan-7-yl]oxy]-4-pyridyl]-N-[5-(trifluoromethyl)-2-pyridyl]pyrazolo[1,5-a]pyridin-2-amine;
5-(5-(((1R,5S,7s)-3-oxa-9-azabicyclo[3.3.1]nonan-7-yl)oxy)-2-methylpyridin-4-yl)-N-(5-(trifluoromethyl)pyrazin-2-yl)pyrazolo[1,5-a]pyridin-2-amine;
6-[[5-[2-methyl-5-[[(1R,5S,7s)-3-oxa-9-azabicyclo[3.3.1]nonan-7-yl]oxy]-4-pyridyl]pyrazolo[1,5-a]pyridin-2-yl]amino]pyridine-3-carboxamide;
6-[[5-[2-methyl-5-[[(1R,5S,7s)-3-oxa-9-azabicyclo[3.3.1]nonan-7-yl]oxy]-4-pyridyl]pyrazolo[1,5-a]pyridin-2-yl]amino]-N-propyl-pyridine-3-carboxamide;
5-[2-methyl-5-[[(1S,5R,7s)-3-oxa-9-azabicyclo[3.3.1]nonan-7-yl]oxy]-4-pyridyl]-N-[4-(trifluoromethyl)-2-pyridyl]pyrazolo[1,5-a]pyridin-2-amine;
5-[2-methyl-5-[[(1S,5R,7s)-3-oxa-9-azabicyclo[3.3.1]nonan-7-yl]oxy]-4-pyridyl]-N-[1-methyl-5-(trifluoromethyl)pyrazol-3-yl]pyrazolo[1,5-a]pyridin-2-amine;
5-[2-methyl-5-[[(1R,5S,7s)-3-oxa-9-azabicyclo[3.3.1]nonan-7-yl]oxy]-4-pyridyl]-N-(1-methyltriazol-4-yl)pyrazolo[1,5-a]pyridin-2-amine;
5-[2-methyl-5-[[(1S,5R,7s)-3-oxa-9-azabicyclo[3.3.1]nonan-7-yl]oxy]-4-pyridyl]-N-(5-methylpyrazin-2-yl)pyrazolo[1,5-a]pyridin-2-amine;
N-(2,6-dimethylpyrimidin-4-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine;
N,N-dimethyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
N-ethyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
N-ethyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridazine-3-carboxamide;
N-ethyl-5-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyrazine-2-carboxamide;
N-(2-methoxyethyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]-N-tetrahydropyran-4-yl-pyridine-3-carboxamide;
N-(2-cyano-2-methyl-propyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
N-(2-hydroxy-2-methyl-propyl)-5-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyrazine-2-carboxamide;
5-(5-(((1R,5S,7s)-3-oxa-9-azabicyclo[3.3.1]nonan-7-yl)oxy)-2-methylpyridin-4-yl)-N-(5-methylpyrimidin-2-yl)pyrazolo[1,5-a]pyridin-2-amine;
5-[2-methyl-5-[[(1S,5R,7s)-3-oxa-9-azabicyclo[3.3.1]nonan-7-yl]oxy]-4-pyridyl]-N-pyrazin-2-yl-pyrazolo[1,5-a]pyridin-2-amine;
5-[2-methyl-5-[[(1R,5S,7s)-3-oxa-9-azabicyclo[3.3.1]nonan-7-yl]oxy]-4-pyridyl]-N-(5-methyl sulfonyl-2-pyridyl)pyrazolo[1,5-a]pyridin-2-amine
5-[2-methyl-5-[[(1R,5S,7s)-3-oxa-9-azabicyclo[3.3.1]nonan-7-yl]oxy]-4-pyridyl]-N-pyridazin-3-yl-pyrazolo[1,5-a]pyridin-2-amine;
5-[2-methyl-5-[[(1R,5S,7s)-3-oxa-9-azabicyclo[3.3.1]nonan-7-yl]oxy]-4-pyridyl]-N-pyrimidin-4-yl-pyrazolo[1,5-a]pyridin-2-amine;
5-[2-methyl-5-[[(1R,5S,7s)-3-oxa-9-azabicyclo[3.3.1]nonan-7-yl]oxy]-4-pyridyl]-N-(6-methylpyrimidin-4-yl)pyrazolo[1,5-a]pyridin-2-amine;
N-(5-fluoro-1-methyl-pyrazol-3-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine;
N-(3-fluoro-1-methyl-pyrazol-4-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine;
N-(4-fluoro-1-methyl-pyrazol-3-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine;
N-[3-methyl-5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]cyclopropanecarboxamide;
2-methyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridazin-3-one;
5-[2-methyl-5-[[(1S,5R,7s)-3-oxa-9-azabicyclo[3.3.1]nonan-7-yl]oxy]-4-pyridyl]-N-pyrimidin-2-yl-pyrazolo[1,5-a]pyridin-2-amine;
5-(5-(((1R,5S,7s)-3-oxa-9-azabicyclo[3.3.1]nonan-7-yl)oxy)-2-methylpyridin-4-yl)-N-(1-methyl-3-(trifluoromethyl)-1H-pyrazol-5-yl)pyrazolo[1,5-a]pyridin-2-amine;
N-(5-fluoropyrimidin-2-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine;
5-(5-(((1R,5S,7s)-3-oxa-9-azabicyclo[3.3.1]nonan-7-yl)oxy)-2-methylpyridin-4-yl)pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-[6-(trifluoromethyl)-2-pyridyl]pyrazolo[1,5-a]pyridin-2-amine;
N-methyl-6-[[5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-yl]amino]pyridine-2-carboxamide;
5-[2-methyl-5-[[(1S,5R,7s)-9-methyl-3-oxa-9-azabicyclo[3.3.1]nonan-7-yl]oxy]-4-pyridyl]-N-pyridazin-4-yl-pyrazolo[1,5-a]pyridin-2-amine;
N-methyl-2-[[5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-yl]amino]pyridine-4-carboxamide;
5-[2-methyl-5-[[(1R,5S,7s)-9-methyl-3-oxa-9-azabicyclo[3.3.1]nonan-7-yl]oxy]-4-pyridyl]-N-(2-methylpyrimidin-4-yl)pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-[5-(trifluoromethyl)-2-pyridyl]pyrazolo[1,5-a]pyridin-2-amine;
N-(3-fluoro-1-methyl-pyrazol-4-yl)-5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-amine;
5-[2-methyl-5-[[(1R,5S,7s)-9-methyl-3-oxa-9-azabicyclo[3.3.1]nonan-7-yl]oxy]-4-pyridyl]-N-[4-(trifluoromethyl)-2-pyridyl]pyrazolo[1,5-a]pyridin-2-amine;
5-[2-methyl-5-[[(1R,5S,7s)-9-methyl-3-oxa-9-azabicyclo[3.3.1]nonan-7-yl]oxy]-4-pyridyl]-N-(1-methyltriazol-4-yl)pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-pyrimidin-5-yl-pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-(5-methylpyrazin-2-yl)pyrazolo[1,5-a]pyridin-2-amine;
N-(2,6-dimethylpyrimidin-4-yl)-5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-pyrazin-2-yl-pyrazolo[1,5-a]pyridin-2-amine;
5-[2-methyl-5-[[(1R,5S,7s)-9-methyl-3-oxa-9-azabicyclo[3.3.1]nonan-7-yl]oxy]-4-pyridyl]-N-pyridazin-3-yl-pyrazolo[1,5-a]pyridin-2-amine;
5-[2-methyl-5-[[(1R,5S,7s)-9-methyl-3-oxa-9-azabicyclo[3.3.1]nonan-7-yl]oxy]-4-pyridyl]-N-pyrimidin-4-yl-pyrazolo[1,5-a]pyridin-2-amine;
5-[2-methyl-5-[[(1R,5S,7s)-9-methyl-3-oxa-9-azabicyclo[3.3.1]nonan-7-yl]oxy]-4-pyridyl]-N-(6-methylpyrimidin-4-yl)pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-(1-methylpyrazol-3-yl)pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-(1-methylpyrazol-3-yl)pyrazolo[1,5-a]pyridin-2-amine;
5-[2-methyl-5-[[(2S)-morpholin-2-yl]methoxy]-4-pyridyl]-N-[5-(trifluoromethyl)-2-pyridyl]pyrazolo[1,5-a]pyridin-2-amine;
5-[2-methyl-5-[[(2S)-morpholin-2-yl]methoxy]-4-pyridyl]-N-(5-methyl-2-pyridyl)pyrazolo[1,5-a]pyridin-2-amine;
5-[2-methyl-5-[[(2S)-morpholin-2-yl]methoxy]-4-pyridyl]-N-(6-methyl-2-pyridyl)pyrazolo[1,5-a]pyridin-2-amine;
5-[2-methyl-5-[[(2S)-morpholin-2-yl]methoxy]-4-pyridyl]-N-(4-methyl-2-pyridyl)pyrazolo[1,5-a]pyridin-2-amine;
5-[2-methyl-5-[[(2R)-morpholin-2-yl]methoxy]-4-pyridyl]-N-(2-pyridyl)pyrazolo[1,5-a]pyridin-2-amine;
5-[2-methyl-5-[[(2R)-morpholin-2-yl]methoxy]-4-pyridyl]-N-(1-methylpyrazol-3-yl)pyrazolo[1,5-a]pyridin-2-amine;
5-[2-methyl-5-[[(2R)-morpholin-2-yl]methoxy]-4-pyridyl]-N-pyrazin-2-yl-pyrazolo[1,5-a]pyridin-2-amine;
5-[2-methyl-5-[[(2S)-morpholin-2-yl]methoxy]-4-pyridyl]-N-pyrazin-2-yl-pyrazolo[1,5-a]pyridin-2-amine;
N-(2,6-dimethylpyrimidin-4-yl)-5-[2-methyl-5-[[(2S)-morpholin-2-yl]methoxy]-4-pyridyl]pyrazolo[1,5-a]pyridin-2-amine;
N-[5-[5-[(3-aminooxetan-3-yl)methoxy]-2-prop-1-ynyl-4-pyridyl]pyrazolo[1,5-a]pyridin-2-yl]cyclopropanecarboxamide;
N-[5-[5-[(3-fluoroazetidin-3-yl)methoxy]-2-prop-1-ynyl-4-pyridyl]pyrazolo[1,5-a]pyridin-2-yl]cyclopropanecarboxamide;
N-(5-(5-((1r,3r)-3-aminocyclobutoxy)-2-(prop-1-yn-1-yl)pyridin-4-yl)pyrazolo[1,5-a]pyridin-2-yl)cyclopropanecarboxamide;
N-[5-[5-[[(1S,5R)-3-oxa-9-azabicyclo[3.3.1]nonan-7-yl]oxy]-2-prop-1-ynyl-4-pyridyl]pyrazolo[1,5-a]pyridin-2-yl]cyclopropanecarboxamide;
5-[5-[(3-fluoroazetidin-3-yl)methoxy]-2-prop-1-ynyl-4-pyridyl]-N-(1-methylpyrazol-3-yl)pyrazolo[1,5-a]pyridin-2-amine;
N-(1-methylpyrazol-3-yl)-5-[5-[[(1S,5R)-3-oxa-9-azabicyclo[3.3.1]nonan-7-yl]oxy]-2-prop-1-ynyl-4-pyridyl]pyrazolo[1,5-a]pyridin-2-amine;
N-[5-[5-[[(1S,5R)-3-oxa-9-azabicyclo[3.3.1]nonan-7-yl]oxy]-2-prop-1-ynyl-4-pyridyl]pyrazolo[1,5-a]pyridin-2-yl]cyclopropanecarboxamide;
N-(2,6-dimethylpyrimidin-4-yl)-5-[5-[[(1S,4S,7R)-2-oxa-5-azabicyclo[2.2.1]heptan-7-yl]oxy]-2-prop-1-ynyl-4-pyridyl]pyrazolo[1,5-a]pyridin-2-amine;
N-[5-[5-[[(3R)-1-methylpyrrolidin-3-yl]methoxy]-2-prop-1-ynyl-4-pyridyl]pyrazolo[1,5-a]pyridin-2-yl]cyclopropanecarboxamide;
N-[5-[5-(1-methylazetidin-3-yl)oxy-2-prop-1-ynyl-4-pyridyl]pyrazolo[1,5-a]pyridin-2-yl]cyclopropanecarboxamide;
N-[5-[5-[[(2S)-morpholin-2-yl]methoxy]-2-prop-1-ynyl-4-pyridyl]pyrazolo[1,5-a]pyridin-2-yl]cyclopropanecarboxamide;
N-(5-(5-((1r,3r)-3-amino-3-methylcyclobutoxy)-2-methylpyridin-4-yl)pyrazolo[1,5-a]pyridin-2-yl)cyclopropanecarboxamide;
N-[5-[2-methyl-5-[[(3*S,5*R)-5-(trifluoromethyl)-3-piperidyl]oxy]-4-pyridyl]pyrazolo[1,5-a]pyridin-2-yl]cyclopropanecarboxamide;
N-[5-[2-methyl-5-[[(3*R,5*S)-5-(trifluoromethyl)-3-piperidyl]oxy]-4-pyridyl]pyrazolo[1,5-a]pyridin-2-yl]cyclopropanecarboxamide;
(*R)—N-[5-[5-[(3-fluoropyrrolidin-3-yl)methoxy]-2-methyl-4-pyridyl]pyrazolo[1,5-a]pyridin-2-yl]cyclopropanecarboxamide;
(*S)—N-[5-[5-[(3-fluoropyrrolidin-3-yl)methoxy]-2-methyl-4-pyridyl]pyrazolo[1,5-a]pyridin-2-yl]cyclopropanecarboxamide;
*R—N-[5-[5-[(3-fluoro-1-methyl-pyrrolidin-3-yl)methoxy]-2-methyl-4-pyridyl]pyrazolo[1,5-a]pyridin-2-yl]cyclopropanecarboxamide;
(*S)—N-[5-[5-[(1,2-dimethylazetidin-2-yl)methoxy]-2-methyl-4-pyridyl]pyrazolo[1,5-a]pyridin-2-yl]cyclopropanecarboxamide;
N-[5-[5-[[(2R)-1-ethylazetidin-2-yl]methoxy]-2-methyl-4-pyridyl]pyrazolo[1,5-a]pyridin-2-yl]cyclopropanecarboxamide;
N-[5-[2-methyl-5-[[(2R)-1-(2,2,2-trifluoroethyl)azetidin-2-yl]methoxy]-4-pyridyl]pyrazolo[1,5-a]pyridin-2-yl]cyclopropanecarboxamide;
(*S, *S)—N-[5-[5-(2-ethylazetidin-3-yl)oxy-2-methyl-4-pyridyl]pyrazolo[1,5-a]pyridin-2-yl]cyclopropanecarboxamide;
(*R)—N-[5-[2-methyl-5-[(2R)-3,3,3-trifluoro-2-hydroxy-propoxy]-4-pyridyl]pyrazolo[1,5-a]pyridin-2-yl]cyclopropanecarboxamide;
*R, *R—N-[5-[5-(1,2-dimethylazetidin-3-yl)oxy-2-methyl-4-pyridyl]pyrazolo[1,5-a]pyridin-2-yl]cyclopropanecarboxamide;
(*S, *S)—N-[5-[5-(1,2-dimethylazetidin-3-yl)oxy-2-methyl-4-pyridyl]pyrazolo[1,5-a]pyridin-2-yl]cyclopropanecarboxamide;
N-[5-[5-[[1-(2-fluoroethyl)azetidin-3-yl]methoxy]-2-methyl-4-pyridyl]pyrazolo[1,5-a]pyridin-2-yl]cyclopropanecarboxamide;
Trans-*R, *R—N-[5-[5-[(4-methoxypyrrolidin-3-yl)methoxy]-2-methyl-4-pyridyl]pyrazolo[1,5-a]pyridin-2-yl]cyclopropanecarboxamide;
Cis-RS—N-[5-[5-[(4-methoxypyrrolidin-3-yl)methoxy]-2-methyl-4-pyridyl]pyrazolo[1,5-a]pyridin-2-yl]cyclopropanecarboxamide;
N-[5-[2-methyl-5-[[(1S,5R)-9-methyl-3-oxa-9-azabicyclo[3.3.1]nonan-7-yl]oxy]-4-pyridyl]pyrazolo[1,5-a]pyridin-2-yl]cyclopropanecarboxamide;
Trans-*R, *R—N-[5-[5-[(4-methoxy-1-methyl-pyrrolidin-3-yl)methoxy]-2-methyl-4-pyridyl]pyrazolo[1,5-a]pyridin-2-yl]cyclopropanecarboxamide;
N-[5-[2-methyl-5-[[(1S,5R)-9-(trideuteriomethyl)-3-oxa-9-azabicyclo[3.3.1]nonan-7-yl]oxy]-4-pyridyl]pyrazolo[1,5-a]pyridin-2-yl]cyclopropanecarboxamide;
(1R,2R)-2-methyl-N-[5-[2-methyl-5-[[(1S,4S,7R)-2-oxa-5-azabicyclo[2.2.1]heptan-7-yl]oxy]-4-pyridyl]pyrazolo[1,5-a]pyridin-2-yl]cyclopropanecarboxamide;
N-(5-(5-((1s,3s)-3-aminocyclobutoxy)-2-methylpyridin-4-yl)pyrazolo[1,5-a]pyridin-2-yl)cyclopropanecarboxamide;
N-[5-[5-[(2R)-2-amino-2-phenyl-ethoxy]-2-methyl-4-pyridyl]pyrazolo[1,5-a]pyridin-2-yl]cyclopropanecarboxamide;
N-[5-[5-[(1S)-2-amino-1-phenyl-ethoxy]-2-methyl-4-pyridyl]pyrazolo[1,5-a]pyridin-2-yl]cyclopropanecarboxamide;
N-[5-[5-[(2S)-2-amino-2-phenyl-ethoxy]-2-methyl-4-pyridyl]pyrazolo[1,5-a]pyridin-2-yl]cyclopropanecarboxamide;
N-[5-[2-methyl-5-[[(2R)-1-methylazetidin-2-yl]methoxy]-4-pyridyl]pyrazolo[1,5-a]pyridin-2-yl]cyclopropanecarboxamide;
rac-N-[5-[5-[(3,3-difluoroazetidin-2-yl)methoxy]-2-methyl-4-pyridyl]pyrazolo[1,5-a]pyridin-2-yl]cyclopropanecarboxamide;
N-[5-[2-methyl-5-[(3-phenylazetidin-3-yl)methoxy]-4-pyridyl]pyrazolo[1,5-a]pyridin-2-yl]cyclopropanecarboxamide;
N-[5-[5-[[(2S)-3,3-dimethylazetidin-2-yl]methoxy]-2-methyl-4-pyridyl]pyrazolo[1,5-a]pyridin-2-yl]cyclopropanecarboxamide;
Racemic-N-[5-[2-methyl-5-(7-oxa-2-azaspiro[3.5]nonan-3-ylmethoxy)-4-pyridyl]pyrazolo[1,5-a]pyridin-2-yl]cyclopropanecarboxamide;
N-[5-[2-methyl-5-[[(2S)-1,3,3-trimethylazetidin-2-yl]methoxy]-4-pyridyl]pyrazolo[1,5-a]pyridin-2-yl]cyclopropanecarboxamide;
N-[5-[5-methyl-2-[[(2S)-morpholin-2-yl]methoxy]-3-pyridyl]pyrazolo[1,5-a]pyridin-2-yl]cyclopropanecarboxamide;
3-[[6-(difluoromethoxy)-4-[2-([1,2,4]triazolo[1,5-a]pyridin-7-ylamino)pyrazolo[1,5-a]pyridin-5-yl]-3-pyridyl]oxy]-2,2-dimethyl-propanenitrile;
N-[5-[5-cyano-2-[[(3R)-pyrrolidin-3-yl]methoxy]phenyl]pyrazolo[1,5-a]pyridin-2-yl]cyclopropanecarboxamide;
(2*R, 1*S)—N-[5-[5-cyano-2-(1-morpholin-2-ylethoxy)phenyl]pyrazolo[1,5-a]pyridin-2-yl]cyclopropanecarboxamide;
4-[[(2R)-morpholin-2-yl]methoxy]-3-[2-(2-pyridylamino)pyrazolo[1,5-a]pyridin-5-yl]benzonitrile;
N-[5-[2-[[(1R,5S)-3-oxa-9-azabicyclo[3.3.1]nonan-7-yl]oxy]-5-prop-1-ynyl-phenyl]pyrazolo[1,5-a]pyridin-2-yl]cyclopropanecarboxamide;
N-[5-[2-[[(1R,5S)-3-oxa-9-azabicyclo[3.3.1]nonan-7-yl]oxy]-5-propanoyl-phenyl]pyrazolo[1,5-a]pyridin-2-yl]cyclopropanecarboxamide;
N-[5-[5-(difluoromethyl)-2-[[(1R,5S)-3-oxa-9-azabicyclo[3.3.1]nonan-7-yl]oxy]phenyl]pyrazolo[1,5-a]pyridin-2-yl]cyclopropanecarboxamide;
N-[5-[5-(difluoromethyl)-2-[[(1R,5S)-9-methyl-3-oxa-9-azabicyclo[3.3.1]nonan-7-yl]oxy]phenyl]pyrazolo[1,5-a]pyridin-2-yl]cyclopropanecarboxamide;
3-[2-[(2,6-dimethylpyrimidin-4-yl)amino]pyrazolo[1,5-a]pyridin-5-yl]-4-(4-hydroxycyclohexoxy)benzonitrile;
3-[2-[(2,6-dimethylpyrimidin-4-yl)amino]pyrazolo[1,5-a]pyridin-5-yl]-4-(2-hydroxy-2-methyl-propoxy)benzonitrile;
N-[5-[6-chloro-3-(1-methylazetidin-3-yl)oxy-pyridazin-4-yl]pyrazolo[1,5-a]pyridin-2-yl]cyclopropanecarboxamide;
5-[6-methyl-3-[[(1S,5R)-3-oxa-9-azabicyclo[3.3.1]nonan-7-yl]oxy]pyridazin-4-yl]-N-pyrazin-2-yl-pyrazolo[1,5-a]pyridin-2-amine;
[4-[4-[2-[(2,6-dimethylpyrimidin-4-yl)amino]pyrazolo[1,5-a]pyridin-5-yl]-6-methyl-pyridazin-3-yl]oxycyclohexyl]acetate;
4-[4-[2-[(2,6-dimethylpyrimidin-4-yl)amino]pyrazolo[1,5-a]pyridin-5-yl]-6-methyl-pyridazin-3-yl]oxycyclohexanol;
N-[5-[3-[[(3R)-1-methylpyrrolidin-3-yl]methoxy]-4-pyridyl]pyrazolo[1,5-a]pyridin-2-yl]cyclopropanecarboxamide;
N-[5-[2-chloro-5-[[(3R)-1-methylpyrrolidin-3-yl]methoxy]-4-pyridyl]pyrazolo[1,5-a]pyridin-2-yl]cyclopropanecarboxamide;
N-[5-[2-chloro-5-(1-methylazetidin-3-yl)oxy-4-pyridyl]pyrazolo[1,5-a]pyridin-2-yl]cyclopropanecarboxamide;
N-[5-[2-chloro-5-[[(1S,5R)-3-oxa-9-azabicyclo[3.3.1]nonan-7-yl]oxy]-4-pyridyl]pyrazolo[1,5-a]pyridin-2-yl]cyclopropanecarboxamide;
1-[[6-(difluoromethyl)-4-[2-[(2,6-dimethylpyrimidin-4-yl)amino]pyrazolo[1,5-a]pyridin-5-yl]-3-pyridyl]oxy]-2-methyl-propan-2-ol;
3-[[4-[2-[(2,6-dimethylpyrimidin-4-yl)amino]pyrazolo[1,5-a]pyridin-5-yl]-6-(trifluoromethyl)-3-pyridyl]oxy]-2,2-dimethyl-propanenitrile;
5-[2-chloro-5-[[(1S,4S,7R)-2-oxa-5-azabicyclo[2.2.1]heptan-7-yl]oxy]-4-pyridyl]-N-(2,6-dimethylpyrimidin-4-yl)pyrazolo[1,5-a]pyridin-2-amine;
N-(2,6-dimethylpyrimidin-4-yl)-5-[2-methoxy-5-[[(2S)-morpholin-2-yl]methoxy]-4-pyridyl]pyrazolo[1,5-a]pyridin-2-amine;
5-[2-chloro-5-[[(2S)-morpholin-2-yl]methoxy]-4-pyridyl]-N-(2,6-dimethylpyrimidin-4-yl)pyrazolo[1,5-a]pyridin-2-amine;
(2R,3S)-2-[[4-[2-[(2,6-dimethylpyrimidin-4-yl)amino]pyrazolo[1,5-a]pyridin-5-yl]-6-methoxy-3-pyridyl]oxymethyl]tetrahydrofuran-3-ol;
(2R,3S)-2-[[4-[2-[(2,6-dimethylpyrimidin-4-yl)amino]pyrazolo[1,5-a]pyridin-5-yl]-6-(2,2,2-trifluoroethoxy)-3-pyridyl]oxymethyl]tetrahydrofuran-3-ol;
2-[[4-[2-[(2,6-dimethylpyrimidin-4-yl)amino]pyrazolo[1,5-a]pyridin-5-yl]-5-[[(2R,3S)-3-hydroxytetrahydrofuran-2-yl]methoxy]-2-pyridyl]oxy]acetonitrile;
N-(2,6-dimethylpyrimidin-4-yl)-5-[2-(methylamino)-5-[[(2S)-morpholin-2-yl]methoxy]-4-pyridyl]pyrazolo[1,5-a]pyridin-2-amine;
5-[2-(difluoromethoxy)-5-[[(2S)-morpholin-2-yl]methoxy]-4-pyridyl]-N-(2,6-dimethylpyrimidin-4-yl)pyrazolo[1,5-a]pyridin-2-amine;
N-[5-[2-(difluoromethoxy)-5-[[(2S)-morpholin-2-yl]methoxy]-4-pyridyl]pyrazolo[1,5-a]pyridin-2-yl]cyclopropanecarboxamide;
N-[6-[5-(4-hydroxycyclohexoxy)-2-methyl-4-pyridyl]imidazo[1,2-a]pyridin-2-yl]cyclopropanecarboxamide;
N-[6-[2-methyl-5-[[(1S,5R)-3-oxa-9-azabicyclo[3.3.1]nonan-7-yl]oxy]-4-pyridyl]imidazo[1,2-a]pyrazin-2-yl]cyclopropanecarboxamide;
4-[[4-[2-[(2,6-dimethylpyrimidin-4-yl)amino]imidazo[1,2-a]pyrazin-6-yl]-6-methyl-3-pyridyl]oxy]cyclohexanol;
4-[[4-[2-[(2,6-dimethylpyrimidin-4-yl)amino]imidazo[1,2-a]pyrazin-6-yl]-6-methyl-3-pyridyl]oxy]cyclohexanol;
N-[6-[5-(2-cyano-2-methyl-propoxy)-2-methyl-4-pyridyl]imidazo[1,2-a]pyrazin-2-yl]cyclopropanecarboxamide;
N-[5-[2-methyl-5-[[(3R)-1-methylpyrrolidin-3-yl]methoxy]-4-pyridyl]pyrazolo[1,5-a]pyridin-2-yl]cyclopropanecarboxamide;
N-[5-[5-[(3S)-2,2-dimethylazetidin-3-yl]oxy-2-methyl-4-pyridyl]pyrazolo[1,5-a]pyridin-2-yl]cyclopropanecarboxamide;
N-(1-isopropylpyrazol-4-yl)-5-[2-methyl-5-(1-methylazetidin-3-yl)oxy-4-pyridyl]pyrazolo[1,5-a]pyridin-2-amine;
N-[5-[2-methyl-5-[[(2S)-1-methylazetidin-2-yl]methoxy]-4-pyridyl]pyrazolo[1,5-a]pyridin-2-yl]cyclopropanecarboxamide;
5-[2-methyl-5-[[(2S,5R)-5-methylmorpholin-2-yl]methoxy]-4-pyridyl]-N-pyridazin-3-yl-pyrazolo[1,5-a]pyridin-2-amine;
5-[2-methyl-5-[[(1S,5R)-8-methyl-8-azabicyclo[3.2.1]octan-3-yl]oxy]-4-pyridyl]-N-pyridazin-3-yl-pyrazolo[1,5-a]pyridin-2-amine;
5-[2-methyl-5-[[(2S)-4-methylmorpholin-2-yl]methoxy]-4-pyridyl]-N-pyrazin-2-yl-pyrazolo[1,5-a]pyridin-2-amine;
(*R)-5-[5-[(6,6-dimethylmorpholin-2-yl)methoxy]-2-methyl-4-pyridyl]-N-pyridazin-3-yl-pyrazolo[1,5-a]pyridin-2-amine;
N-[5-[5-(3-amino-3-methyl-cyclobutoxy)-2-methyl-4-pyridyl]pyrazolo[1,5-a]pyridin-2-yl]cyclopropanecarboxamide;
5-[5-[[(2S)-6,6-dimethylmorpholin-2-yl]methoxy]-2-methyl-4-pyridyl]-N-pyridazin-3-yl-pyrazolo[1,5-a]pyridin-2-amine;
(*S)—N-[5-[5-[(3-fluoro-1-methyl-pyrrolidin-3-yl)methoxy]-2-methyl-4-pyridyl]pyrazolo[1,5-a]pyridin-2-yl]cyclopropanecarboxamide;
5-[5-(3-aminocyclobutoxy)-2-methyl-4-pyridyl]-N-pyridazin-3-yl-pyrazolo[1,5-a]pyridin-2-amine;
N-(2,6-dimethylpyrimidin-4-yl)-5-[2-methyl-5-[[(2S)-1-methylazetidin-2-yl]methoxy]-4-pyridyl]pyrazolo[1,5-a]pyridin-2-amine;
(*R)—N-[5-[5-[(3-hydroxy-1-methyl-pyrrolidin-3-yl)methoxy]-2-methyl-4-pyridyl]pyrazolo[1,5-a]pyridin-2-yl]cyclopropanecarboxamide;
5-[2-methyl-5-[[(1S,4S,7R)-2-oxa-5-azabicyclo[2.2.1]heptan-7-yl]oxy]-4-pyridyl]-N-(6-methylpyridazin-3-yl)pyrazolo[1,5-a]pyridin-2-amine;
N-(2,6-dimethylpyrimidin-4-yl)-5-[2-methyl-5-[[(1S,4S,7R)-2-oxa-5-azabicyclo[2.2.1]heptan-7-yl]oxy]-4-pyridyl]pyrazolo[1,5-a]pyridin-2-amine;
N-(6-cyclopropylpyridazin-3-yl)-5-[2-methyl-5-[[(1S,4S,7R)-2-oxa-5-azabicyclo[2.2.1]heptan-7-yl]oxy]-4-pyridyl]pyrazolo[1,5-a]pyridin-2-amine;
N-[5-[5-[[(2S)-azetidin-2-yl]methoxy]-2-methyl-4-pyridyl]pyrazolo[1,5-a]pyridin-2-yl]cyclopropanecarboxamide;
N-(2,6-dimethylpyrimidin-4-yl)-5-[2-methyl-5-[[(2R)-1-methylazetidin-2-yl]methoxy]-4-pyridyl]pyrazolo[1,5-a]pyridin-2-amine;
5-[2-methyl-5-[[(1S,4S,7R)-2-oxa-5-azabicyclo[2.2.1]heptan-7-yl]oxy]-4-pyridyl]-N-(5-methylpyrazin-2-yl)pyrazolo[1,5-a]pyridin-2-amine;
N-[5-[5-[[(2R)-1-(2-fluoroethyl)azetidin-2-yl]methoxy]-2-methyl-4-pyridyl]pyrazolo[1,5-a]pyridin-2-yl]cyclopropanecarboxamide;
6-[[5-[5-[[(1S,5R)-9-ethyl-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide;
6-[[5-[5-[[(1S,5R)-9-isopropyl-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide;
6-[[5-[5-[[(1S,5R)-9-(2-hydroxy-2-methyl-propyl)-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide;
(*S)—N-(2,6-dimethylpyrimidin-4-yl)-5-[5-(5,5-dimethyltetrahydrofuran-3-yl)oxy-2-methyl-4-pyridyl]pyrazolo[1,5-a]pyridin-2-amine;
(3*R, 4*R)—N-(2,6-dimethylpyrimidin-4-yl)-5-[2-methyl-5-(4-methyltetrahydrofuran-3-yl)oxy-4-pyridyl]pyrazolo[1,5-a]pyridin-2-amine;
(*R)—N-(2,6-dimethylpyrimidin-4-yl)-5-[2-methyl-5-[(3-methyltetrahydrofuran-3-yl)methoxy]-4-pyridyl]pyrazolo[1,5-a]pyridin-2-amine;
(*S)—N-(2,6-dimethylpyrimidin-4-yl)-5-[2-methyl-5-[(3-methyltetrahydrofuran-3-yl)methoxy]-4-pyridyl]pyrazolo[1,5-a]pyridin-2-amine;
2-methyl-1-[[6-methyl-4-[2-[[6-methyl-2-(trifluoromethyl)pyrimidin-4-yl]amino]pyrazolo[1,5-a]pyridin-5-yl]-3-pyridyl]oxy]propan-2-ol;
(1r,3r)-3-(((4-(2-((2,6-dimethylpyrimidin-4-yl)amino)pyrazolo[1,5-a]pyridin-5-yl)-6-methylpyridin-3-yl)oxy)methyl)cyclobutan-1-ol;
(*R)—N-(2,6-dimethylpyrimidin-4-yl)-5-[5-[(5,5-dimethyltetrahydrofuran-3-yl)methoxy]-2-methyl-4-pyridyl]pyrazolo[1,5-a]pyridin-2-amine;
(*S)—N-(2,6-dimethyl pyrimidin-4-yl)-5-[5-[(5,5-dimethyltetrahydrofuran-3-yl)methoxy]-2-methyl-4-pyridyl]pyrazolo[1,5-a]pyridin-2-amine;
2-methyl-1-[[6-methyl-4-[2-[(5-methylpyrazin-2-yl)amino]pyrazolo[1,5-a]pyridin-5-yl]-3-pyridyl]oxy]propan-2-ol;
(rac-)-4-[[4-[2-[(2,6-dimethylpyrimidin-4-yl)amino]pyrazolo[1,5-a]pyridin-5-yl]-6-methyl-3-pyridyl]oxymethyl]-1-methyl-pyrrolidin-2-one;
(*R)—N-(2,6-dimethylpyrimidin-4-yl)-5-[5-(6,9-dioxaspiro[4.4]nonan-8-ylmethoxy)-2-methyl-4-pyridyl]pyrazolo[1,5-a]pyridin-2-amine;
(*S)—N-(2,6-dimethylpyrimidin-4-yl)-5-[5-(6,9-dioxaspiro[4.4]nonan-8-ylmethoxy)-2-methyl-4-pyridyl]pyrazolo[1,5-a]pyridin-2-amine;
5-(5-((1r,3r)-3-aminocyclobutoxy)-2-methylpyridin-4-yl)-N-(2,6-dimethylpyrimidin-4-yl)pyrazolo[1,5-a]pyridin-2-amine;
N-(2,6-dimethyl pyrimidin-4-yl)-5-[2-methyl-5-[(3-methyl oxetan-3-yl)methoxy]-4-pyridyl]pyrazolo[1,5-a]pyridin-2-amine;
(*R)-4-[[4-[2-[(2,6-dimethylpyrimidin-4-yl)amino]pyrazolo[1,5-a]pyridin-5-yl]-6-methyl-3-pyridyl]oxymethyl]-1-methyl-pyrrolidin-2-one;
(*R)-2-[[4-[2-[(2,6-dimethylpyrimidin-4-yl)amino]pyrazolo[1,5-a]pyridin-5-yl]-6-methyl-3-pyridyl]oxy]-1-(1-methylcyclopropyl)ethanol;
(*S)-2-[[4-[2-[(2,6-dimethylpyrimidin-4-yl)amino]pyrazolo[1,5-a]pyridin-5-yl]-6-methyl-3-pyridyl]oxy]-1-(1-methylcyclopropyl)ethanol;
N-(2,6-dimethylpyrimidin-4-yl)-5-[5-(1,1-dioxothian-4-yl)oxy-2-methyl-4-pyridyl]pyrazolo[1,5-a]pyridin-2-amine;
N-(2,6-dimethylpyrimidin-4-yl)-5-[2-methyl-5-(4-methylsulfonylcyclohexoxy)-4-pyridyl]pyrazolo[1,5-a]pyridin-2-amine;
4-[[4-[2-[(2,6-dimethylpyrimidin-4-yl)amino]pyrazolo[1,5-a]pyridin-5-yl]-6-methyl-3-pyridyl]oxy]cyclohexanecarbonitrile;
5-[5-[[2-(difluoromethoxy)phenyl]methoxy]-2-methyl-4-pyridyl]-N-(2,6-dimethylpyrimidin-4-yl)pyrazolo[1,5-a]pyridin-2-amine;
(3S)-4-[[4-[2-[(2,6-dimethylpyrimidin-4-yl)amino]pyrazolo[1,5-a]pyridin-5-yl]-6-methyl-3-pyridyl]oxy]-2,3-dimethyl-butan-2-ol;
4-[[4-[2-[(2,6-dimethylpyrimidin-4-yl)amino]pyrazolo[1,5-a]pyridin-5-yl]-6-methyl-3-pyridyl]oxy]-2-methyl-butan-2-ol;
(1s,4s)-4-((4-(2-((2,6-dimethylpyrimidin-4-yl)amino)pyrazolo[1,5-a]pyridin-5-yl)-6-methylpyridin-3-yl)oxy)cyclohexane-1-carbonitrile;
5-[5-(2-aminoethoxy)-2-methyl-4-pyridyl]-N-(2,6-dimethylpyrimidin-4-yl)pyrazolo[1,5-a]pyridin-2-amine;
(1R)-1-cyclopropyl-2-[[4-[2-[(2,6-dimethylpyrimidin-4-yl)amino]pyrazolo[1,5-a]pyridin-5-yl]-6-methyl-3-pyridyl]oxy]ethanol;
(1S)-1-cyclopropyl-2-[[4-[2-[(2,6-dimethylpyrimidin-4-yl)amino]pyrazolo[1,5-a]pyridin-5-yl]-6-methyl-3-pyridyl]oxy]ethanol;
1-[[4-[2-[(2,6-dimethylpyrimidin-4-yl)amino]pyrazolo[1,5-a]pyridin-5-yl]-6-methyl-3-pyridyl]oxymethyl]cyclopropanecarbonitrile;
2-cyclopropyl-6-[[5-[5-[[(2S)-1,4-dioxan-2-yl]methoxy]-2-methyl-4-pyridyl]pyrazolo[1,5-a]pyridin-2-yl]amino]-4-methyl-pyridazin-3-one;
(4S)-4-[[4-[2-[(2,6-dimethylpyrimidin-4-yl)amino]pyrazolo[1,5-a]pyridin-5-yl]-6-methyl-3-pyridyl]oxymethyl]-1,3-dimethyl-imidazolidin-2-one;
N-[5-[2-cyano-5-[[(3R)-1-(cyanomethyl)pyrrolidin-3-yl]methoxy]-4-pyridyl]pyrazolo[1,5-a]pyridin-2-yl]cyclopropanecarboxamide;
5-[2-[(1-methylpyrazol-3-yl)amino]pyrazolo[1,5-a]pyridin-5-yl]-6-[[(2S)-morpholin-2-yl]methoxy]pyridine-3-carbonitrile;
N-[5-[2-cyano-5-[[(1R,5S)-9-methyl-3-oxa-9-azabicyclo[3.3.1]nonan-7-yl]amino]-4-pyridyl]pyrazolo[1,5-a]pyridin-2-yl]cyclopropanecarboxamide;
3-[2-[(1-methylpyrazol-3-yl)amino]pyrazolo[1,5-a]pyridin-5-yl]-4-[[(2R)-morpholin-2-yl]methoxy]benzonitrile;
5-[2-methyl-5-(1-methylazetidin-3-yl)oxy-4-pyridyl]-N-(2-pyridyl)pyrazolo[1,5-a]pyridin-2-amine;
5-[2-methyl-5-(1-methylazetidin-3-yl)oxy-4-pyridyl]-N-(1-methylpyrazol-4-yl)pyrazolo[1,5-a]pyridin-2-amine;
(1*R, 3*R)—N-[5-[5-(3-hydroxycyclopentoxy)-2-methyl-4-pyridyl]pyrazolo[1,5-a]pyridin-2-yl]cyclopropanecarboxamide;
N-[5-[5-[(2R)-2-amino-3,3,3-trifluoro-propoxy]-2-methyl-4-pyridyl]pyrazolo[1,5-a]pyridin-2-yl]cyclopropanecarboxamide;
(3RS, 4RS)—N-[5-[5-[(4-methoxy-1-methyl-pyrrolidin-3-yl)methoxy]-2-methyl-4-pyridyl]pyrazolo[1,5-a]pyridin-2-yl]cyclopropanecarboxamide;
N-[5-[2-methyl-5-[[(2S)-1-methylpyrrolidin-2-yl]methoxy]-4-pyridyl]pyrazolo[1,5-a]pyridin-2-yl]cyclopropanecarboxamide;
N-[5-[5-[[(2S)-1-(2-fluoroethyl)azetidin-2-yl]methoxy]-2-methyl-4-pyridyl]pyrazolo[1,5-a]pyridin-2-yl]cyclopropanecarboxamide;
N-[5-[5-[[(2R)-1-cyclopropylazetidin-2-yl]methoxy]-2-methyl-4-pyridyl]pyrazolo[1,5-a]pyridin-2-yl]cyclopropanecarboxamide;
N-[5-[2-methyl-5-[[(2S)-1-(trideuteriomethyl)azetidin-2-yl]methoxy]-4-pyridyl]pyrazolo[1,5-a]pyridin-2-yl]cyclopropanecarboxamide;
N-[5-[2-methyl-5-[[(2R)-1-(trideuteriomethyl)azetidin-2-yl]methoxy]-4-pyridyl]pyrazolo[1,5-a]pyridin-2-yl]cyclopropanecarboxamide;
(*R)—N-(2,6-dimethylpyrimidin-4-yl)-5-[5-(5,5-dimethyltetrahydrofuran-3-yl)oxy-2-methyl-4-pyridyl]pyrazolo[1,5-a]pyridin-2-amine;
(*S)—N-(2,6-dimethylpyrimidin-4-yl)-5-[5-(2,2-dimethyltetrahydropyran-4-yl)oxy-2-methyl-4-pyridyl]pyrazolo[1,5-a]pyridin-2-amine;
N-[3-[[4-[2-[(2,6-dimethylpyrimidin-4-yl)amino]pyrazolo[1,5-a]pyridin-5-yl]-6-methyl-3-pyridyl]oxy]cyclobutyl]methanesulfonamide;
N-[3-[[4-[2-[(2,6-dimethylpyrimidin-4-yl)amino]pyrazolo[1,5-a]pyridin-5-yl]-6-methyl-3-pyridyl]oxy]cyclobutyl]methanesulfonamide;
(1r,4r)-4-((4-(2-(2,6-dimethylpyrimidin-4-yl)amino)pyrazolo[1,5-a]pyridin-5-yl)-6-methylpyridin-3-yl)oxy)cyclohexane-1-carbonitrile;
(3*R, 6*R)-6-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol;
N-[5-(2-cyano-5-ethoxy-4-pyridyl)pyrazolo[1,5-a]pyridin-2-yl]cyclopropanecarboxamide;
N-[5-[2-cyano-5-(cyclopropylmethoxy)-4-pyridyl]pyrazolo[1,5-a]pyridin-2-yl]cyclopropanecarboxamide;
N-[5-[2-cyano-5-[[(3R)-3-piperidyl]methoxy]-4-pyridyl]pyrazolo[1,5-a]pyridin-2-yl]cyclopropanecarboxamide;
N-(5-(5-(((1r,4r)-4-aminocyclohexyl)methoxy)-2-cyanopyridin-4-yl)pyrazolo[1,5-a]pyridin-2-yl)cyclopropanecarboxamide;
N-[5-[5-(6-azaspiro[3.3]heptan-2-yloxy)-2-cyano-4-pyridyl]pyrazolo[1,5-a]pyridin-2-yl]cyclopropanecarboxamide;
N-[5-[2-cyano-5-[[(3R)-1-(2,2,2-trifluoroethyl)pyrrolidin-3-yl]methoxy]-4-pyridyl]pyrazolo[1,5-a]pyridin-2-yl]cyclopropanecarboxamide;
N-[5-[2-cyano-5-[[(3R)-1-cyclopropylpyrrolidin-3-yl]methoxy]-4-pyridyl]pyrazolo[1,5-a]pyridin-2-yl]cyclopropanecarboxamide;
N-[5-[2-cyano-5-[1-(2,2,2-trifluoroethyl)azetidin-3-yl]oxy-4-pyridyl]pyrazolo[1,5-a]pyridin-2-yl]cyclopropanecarboxamide;
N-(5-(5-(((1r,3r)-3-aminocyclobutyl)methoxy)-2-cyanopyridin-4-yl)pyrazolo[1,5-a]pyridin-2-yl)cyclopropanecarboxamide;
N-[5-[2-cyano-5-[[(2R)-morpholin-2-yl]methoxy]-4-pyridyl]pyrazolo[1,5-a]pyridin-2-yl]cyclopropanecarboxamide;
N-[5-[2-cyano-5-[[(3S)-3-piperidyl]methoxy]-4-pyridyl]pyrazolo[1,5-a]pyridin-2-yl]cyclopropanecarboxamide;
N-[5-[2-cyano-5-[(3R)-pyrrolidin-3-yl]oxy-4-pyridyl]pyrazolo[1,5-a]pyridin-2-yl]cyclopropanecarboxamide;
(1*S, 4*S, 5*R)—N-[5-[2-cyano-5-[(2-methyl-2-azabicyclo[2.2.1]heptan-5-yl)oxy]-4-pyridyl]pyrazolo[1,5-a]pyridin-2-yl]cyclopropanecarboxamide;
N-[5-[2-cyano-5-[[3-(cyanomethylamino)cyclobutyl]methoxy]-4-pyridyl]pyrazolo[1,5-a]pyridin-2-yl]cyclopropanecarboxamide;
N-[5-[2-cyano-5-[[3-(cyclopropylamino)cyclobutyl]methoxy]-4-pyridyl]pyrazolo[1,5-a]pyridin-2-yl]cyclopropanecarboxamide;
ethyl 2-[[3-[[6-cyano-4-[2-(cyclopropanecarbonylamino)pyrazolo[1,5-a]pyridin-5-yl]-3-pyridyl]oxymethyl]cyclobutyl]amino]acetate;
N-[5-[5-[[3-(benzylamino)cyclobutyl]methoxy]-2-cyano-4-pyridyl]pyrazolo[1,5-a]pyridin-2-yl]cyclopropanecarboxamide;
1-[4-[2-[(2,6-dimethylpyrimidin-4-yl)amino]pyrazolo[1,5-a]pyridin-5-yl]-6-methyl-pyridazin-3-yl]oxy-2-methyl-propan-2-ol;
N-[5-[5-[[(2S)-morpholin-2-yl]methoxy]-2-(trifluoromethyl)-4-pyridyl]pyrazolo[1,5-a]pyridin-2-yl]cyclopropanecarboxamide;
(1*R, 3*S)—N-[5-[5-(3-hydroxycyclohexoxy)-2-methyl-4-pyridyl]pyrazolo[1,5-a]pyridin-2-yl]cyclopropanecarboxamide;
(*S)—N-[5-[5-(2-cyclopropyl-2-hydroxy-propoxy)-2-methyl-4-pyridyl]pyrazolo[1,5-a]pyridin-2-yl]cyclopropanecarboxamide;
(*R)—N-[5-[5-[(1,2-dimethylazetidin-2-yl)methoxy]-2-methyl-4-pyridyl]pyrazolo[1,5-a]pyridin-2-yl]cyclopropanecarboxamide;
N-[5-[5-[[(2S)-1-ethylazetidin-2-yl]methoxy]-2-methyl-4-pyridyl]pyrazolo[1,5-a]pyridin-2-yl]cyclopropanecarboxamide;
(3S,5S)-5-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol;
(*R)—N-(2,6-dimethylpyrimidin-4-yl)-5-[2-methyl-5-(7-oxaspiro[3.4]octan-6-ylmethoxy)-4-pyridyl]pyrazolo[1,5-a]pyridin-2-amine;
(*S)-3-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol;
(3S,5R)-5-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol;
(*S)—N-(2,6-dimethylpyrimidin-4-yl)-5-[5-(1,1-dioxothiazinan-4-yl)oxy-2-methyl-4-pyridyl]pyrazolo[1,5-a]pyridin-2-amine;
5-[2-methyl-5-[[(2R)-4-methylmorpholin-2-yl]methoxy]-4-pyridyl]-N-pyrazin-2-yl-pyrazolo[1,5-a]pyridin-2-amine;
2-methyl-1-[[6-methyl-4-[2-[(6-methylpyridazin-3-yl)amino]pyrazolo[1,5-a]pyridin-5-yl]-3-pyridyl]oxy]propan-2-ol;
1-[[4-[2-[(5-tert-butylpyrazin-2-yl)amino]pyrazolo[1,5-a]pyridin-5-yl]-6-methyl-3-pyridyl]oxy]-2-methyl-propan-2-ol;
N-cyclopropyl-2,6-difluoro-4-[[5-[5-(4-hydroxycyclohexoxy)-2-methyl-4-pyridyl]pyrazolo[1,5-a]pyridin-2-yl]amino]benzamide;
N-cyclopropyl-2-fluoro-4-[[5-[5-(2-hydroxy-2-methyl-propoxy)-2-methyl-4-pyridyl]pyrazolo[1,5-a]pyridin-2-yl]amino]benzamide;
N-[5-[2-cyano-5-[[(3R)-1-ethylpyrrolidin-3-yl]methoxy]-4-pyridyl]pyrazolo[1,5-a]pyridin-2-yl]cyclopropanecarboxamide;
N-(6-(5-(((1R,5S,7s)-3-oxa-9-azabicyclo[3.3.1]nonan-7-yl)oxy)-2-methylpyridin-4-yl)imidazo[1,2-a]pyridin-2-yl)cyclopropanecarboxamide;
N-(5-(5-(((1R,5S,7s)-3-oxa-9-azabicyclo[3.3.1]nonan-7-yl)oxy)-2-(trifluoromethyl)pyridin-4-yl)pyrazolo[1,5-a]pyridin-2-yl)cyclopropanecarboxamide;
N-(5-(5-(((1R,5S,7s)-9-methyl-3-oxa-9-azabicyclo[3.3.1]nonan-7-yl)oxy)-2-(trifluoromethyl)pyridin-4-yl)pyrazolo[1,5-a]pyridin-2-yl)cyclopropanecarboxamide;
N-[5-[5-[(1-amino-3,3-difluoro-cyclobutyl)methoxy]-2-(trifluoromethyl)-4-pyridyl]pyrazolo[1,5-a]pyridin-2-yl]cyclopropanecarboxamide;
N-[5-[5-[(3-fluoroazetidin-3-yl)methoxy]-2-(trifluoromethyl)-4-pyridyl]pyrazolo[1,5-a]pyridin-2-yl]cyclopropanecarboxamide;
N-[5-[5-[(3-methoxyazetidin-3-yl)methoxy]-2-(trifluoromethyl)-4-pyridyl]pyrazolo[1,5-a]pyridin-2-yl]cyclopropanecarboxamide;
1-fluoro-N-[5-[2-methyl-5-[[(2R)-1-methylazetidin-2-yl]methoxy]-4-pyridyl]pyrazolo[1,5-a]pyridin-2-yl]cyclopropanecarboxamide;
2,2-difluoro-N-[5-[2-methyl-5-[[(2R)-1-methylazetidin-2-yl]methoxy]-4-pyridyl]pyrazolo[1,5-a]pyridin-2-yl]cyclopropanecarboxamide;
N-(2,6-dimethylpyrimidin-4-yl)-5-[5-[[(2R)-1-methylazetidin-2-yl]methoxy]-2-(trifluoromethyl)-4-pyridyl]pyrazolo[1,5-a]pyridin-2-amine;
N-cyclopropyl-4-[[5-[5-(2-hydroxy-2-methyl-propoxy)-2-methyl-4-pyridyl]pyrazolo[1,5-a]pyridin-2-yl]amino]-2,6-dimethoxy-benzamide;
1-((1R,5S,7s)-7-((4-(2-((2,6-dimethylpyrimidin-4-yl)amino)pyrazolo[1,5-a]pyridin-5-yl)-6-methylpyridin-3-yl)oxy)-3-oxa-9-azabicyclo[3.3.1]nonan-9-yl)ethan-1-one;
1-[(2S)-2-[[4-[2-[(2,6-dimethylpyrimidin-4-yl)amino]pyrazolo[1,5-a]pyridin-5-yl]-6-methyl-3-pyridyl]oxymethyl]morpholin-4-yl]ethanone;
N-(2,6-dimethylpyrimidin-4-yl)-5-[2-methyl-5-[[(2S)-4-methylsulfonylmorpholin-2-yl]methoxy]-4-pyridyl]pyrazolo[1,5-a]pyridin-2-amine;
(S)-5-(2-methyl-5-((4-(methylsulfonyl)morpholin-2-yl)methoxy)pyridin-4-yl)-N-(2-methylpyrimidin-4-yl)pyrazolo[1,5-a]pyridin-2-amine;
2-((5-(5-(((1R,5S,7s)-9-acetyl-3-oxa-9-azabicyclo[3.3.1]nonan-7-yl)oxy)-2-methylpyridin-4-yl)pyrazolo[1,5-a]pyridin-2-yl)amino)-N-methylisonicotinamide;
1-((1R,5S,7s)-7-((6-methyl-4-(2-((4-(trifluoromethyl)pyridin-2-yl)amino)pyrazolo[1,5-a]pyridin-5-yl)pyridin-3-yl)oxy)-3-oxa-9-azabicyclo[3.3.1]nonan-9-yl)ethan-1-one;
2-[[5-[5-(2-hydroxy-2-methyl-propoxy)-2-methyl-4-pyridyl]pyrazolo[1,5-a]pyridin-2-yl]amino]-N,N,6-trimethyl-pyridine-4-carboxamide;
2-((5-(5-(((1R,5S,7s)-9-(cyclopropanecarbonyl)-3-oxa-9-azabicyclo[3.3.1]nonan-7-yl)oxy)-2-methylpyridin-4-yl)pyrazolo[1,5-a]pyridin-2-yl)amino)-N-methylisonicotinamide;
2-((5-(5-(((1R,5S,7s)-9-(cyclopropyl sulfonyl)-3-oxa-9-azabicyclo[3.3.1]nonan-7-yl)oxy)-2-methylpyridin-4-yl)pyrazolo[1,5-a]pyridin-2-yl)amino)-N-methylisonicotinamide;
N-cyclopropyl-2-[[5-[5-(2-hydroxy-2-methyl-propoxy)-2-methyl-4-pyridyl]pyrazolo[1,5-a]pyridin-2-yl]amino]-6-methyl-pyridine-4-carboxamide;
6-((5-(5-(((1R,5S,7s)-9-acetyl-3-oxa-9-azabicyclo[3.3.1]nonan-7-yl)oxy)-2-methylpyridin-4-yl)pyrazolo[1,5-a]pyridin-2-yl)amino)-2-methylpyridazin-3(2H)-one;
N-cyclopropyl-6-[[5-[5-(2-hydroxy-2-methyl-propoxy)-2-methyl-4-pyridyl]pyrazolo[1,5-a]pyridin-2-yl]amino]-4-methyl-pyridine-2-carboxamide;
N-cyclopropyl-6-[[5-[5-(2-hydroxy-2-methyl-propoxy)-2-methyl-4-pyridyl]pyrazolo[1,5-a]pyridin-2-yl]amino]pyridine-3-carboxamide;
N-cyclopropyl-4-[[5-[5-(2-hydroxy-2-methyl-propoxy)-2-methyl-4-pyridyl]pyrazolo[1,5-a]pyridin-2-yl]amino]-6-methyl-pyrimidine-2-carboxamide;
3-[[6-(difluoromethoxy)-4-[2-[(2-methylpyrimidin-5-yl)amino]pyrazolo[1,5-a]pyridin-5-yl]-3-pyridyl]oxy]-2,2-dimethyl-propanenitrile;
methyl 4-[[5-[5-(2-hydroxy-2-methyl-propoxy)-2-methyl-4-pyridyl]pyrazolo[1,5-a]pyridin-2-yl]amino]-2-methoxy-benzoate;
N-cyclopropyl-4-[[5-[5-(2-hydroxy-2-methyl-propoxy)-2-methyl-4-pyridyl]pyrazolo[1,5-a]pyridin-2-yl]amino]-2-methoxy-benzamide;
4-[[5-[5-(2-hydroxy-2-methyl-propoxy)-2-methyl-4-pyridyl]pyrazolo[1,5-a]pyridin-2-yl]amino]-6-methyl-N-(2,2,2-trifluoroethyl)pyrimidine-2-carboxamide;
(1r,4r)-4-((4-(2-(3,5-dimethoxyphenyl)amino)pyrazolo[1,5-a]pyridin-5-yl)-6-methylpyridin-3-yl)oxy)cyclohexan-1-ol;
1-[[4-[2-(3,5-dimethoxyanilino)pyrazolo[1,5-a]pyridin-5-yl]-6-methyl-3-pyridyl]oxy]-2-methyl-propan-2-ol;
4-[[5-[5-(2-hydroxy-2-methyl-propoxy)-2-methyl-4-pyridyl]pyrazolo[1,5-a]pyridin-2-yl]amino]-N-(2-hydroxy-2-methyl-propyl)-6-methyl-pyrimidine-2-carboxamide;
N-cyclopropyl-2-(difluoromethoxy)-4-[[5-[5-(2-hydroxy-2-methyl-propoxy)-2-methyl-4-pyridyl]pyrazolo[1,5-a]pyridin-2-yl]amino]-6-methoxy-benzamide;
N-cyclopropyl-2-fluoro-4-((5-(5-(((1r,4r)-4-hydroxycyclohexyl)oxy)-2-methylpyridin-4-yl)pyrazolo[1,5-a]pyridin-2-yl)amino)-6-methoxybenzamide;
6-[[5-[5-(2-hydroxy-2-methyl-propoxy)-2-methyl-4-pyridyl]pyrazolo[1,5-a]pyridin-2-yl]amino]-N-isopropyl-4-(trifluoromethyl)pyridine-3-carboxamide;
6-[[5-[5-(2-hydroxy-2-methyl-propoxy)-2-methyl-4-pyridyl]pyrazolo[1,5-a]pyridin-2-yl]amino]-N-(2,2,2-trifluoroethyl)-4-(trifluoromethyl)pyridine-3-carboxamide;
1-[[4-[2-[(3-fluoro-4-methyl-2-pyridyl)amino]pyrazolo[1,5-a]pyridin-5-yl]-6-methyl-3-pyridyl]oxy]-2-methyl-propan-2-ol;
N-[6-[5-(4-hydroxycyclohexoxy)-2-methyl-4-pyridyl]imidazo[1,2-a]pyrazin-2-yl]cyclopropanecarboxamide;
N-[6-[2-methyl-5-(1-methylazetidin-3-yl)oxy-4-pyridyl]imidazo[1,2-a]pyrazin-2-yl]cyclopropanecarboxamide;
N-[6-[5-(4-hydroxycyclohexoxy)-2-methyl-4-pyridyl]imidazo[1,2-a]pyrazin-2-yl]cyclopropanecarboxamide;
N-[5-[2-cyano-5-[[(1R,5S,7s)-9-methyl-3-oxa-9-azabicyclo[3.3.1]nonan-7-yl]oxy]-4-pyridyl]pyrazolo[1,5-a]pyridin-2-yl]cyclopropanecarboxamide;
4-[[6-methyl-4-[2-[(6-methylpyrazin-2-yl)amino]imidazo[1,2-a]pyrazin-6-yl]-3-pyridyl]oxy]cyclohexanol;
N-(2,6-dimethylpyrimidin-4-yl)-5-[2-ethoxy-5-[[(2S)-morpholin-2-yl]methoxy]-4-pyridyl]pyrazolo[1,5-a]pyridin-2-amine;
3-[[4-[2-[(2,6-dimethylpyrimidin-4-yl)amino]pyrazolo[1,5-a]pyridin-5-yl]-6-(2,2,2-trifluoroethoxy)-3-pyridyl]oxy]-2,2-dimethyl-propanenitrile;
3-[[6-(2,2-difluoroethoxy)-4-[2-[(2,6-dimethylpyrimidin-4-yl)amino]pyrazolo[1,5-a]pyridin-5-yl]-3-pyridyl]oxy]-2,2-dimethyl-propanenitrile;
1-[[4-[2-[(2,6-dimethylpyrimidin-4-yl)amino]pyrazolo[1,5-a]pyridin-5-yl]-6-(2,2,2-trifluoroethoxy)-3-pyridyl]oxy]-2-methyl-propan-2-ol;
N-(2,6-dimethylpyrimidin-4-yl)-5-[5-[[(2S)-morpholin-2-yl]methoxy]-2-(2,2,2-trifluoroethoxy)-4-pyridyl]pyrazolo[1,5-a]pyridin-2-amine;
3-[[4-[2-[(2,6-dimethylpyrimidin-4-yl)amino]pyrazolo[1,5-a]pyridin-5-yl]-6-isopropoxy-3-pyridyl]oxy]-2,2-dimethyl-propanenitrile;
4-[[4-[2-[(2,6-dimethylpyrimidin-4-yl)amino]pyrazolo[1,5-a]pyridin-5-yl]-6-(2,2,2-trifluoroethoxy)-3-pyridyl]oxy]cyclohexanol;
N-[5-[5-(2-cyano-2-methyl-propoxy)-2-(difluoromethoxy)-4-pyridyl]pyrazolo[1,5-a]pyridin-2-yl]cyclopropanecarboxamide;
N-[5-[2-chloro-5-(2-cyano-2-methyl-propoxy)-4-pyridyl]pyrazolo[1,5-a]pyridin-2-yl]cyclopropanecarboxamide;
3-[[6-(2,2-difluoroethylamino)-4-[2-[(2,6-dimethylpyrimidin-4-yl)amino]pyrazolo[1,5-a]pyridin-5-yl]-3-pyridyl]oxy]-2,2-dimethyl-propanenitrile;
3-[[4-[2-[(2,6-dimethylpyrimidin-4-yl)amino]pyrazolo[1,5-a]pyridin-5-yl]-6-(methoxymethyl)-3-pyridyl]oxy]-2,2-dimethyl-propanenitrile;
N-[5-[5-(2-cyano-2-methyl-propoxy)-2-(2,2,2-trifluoroethoxy)-4-pyridyl]pyrazolo[1,5-a]pyridin-2-yl]cyclopropanecarboxamide;
3-[[4-[2-[(2,6-dimethylpyrimidin-4-yl)amino]pyrazolo[1,5-a]pyridin-5-yl]-6-prop-1-ynyl-3-pyridyl]oxy]-2,2-dimethyl-propanenitrile;
(2S,3R)-2-[[4-[2-[(2,6-dimethylpyrimidin-4-yl)amino]pyrazolo[1,5-a]pyridin-5-yl]-6-prop-1-ynyl-3-pyridyl]oxymethyl]tetrahydrofuran-3-ol;
3-[[6-(cyanomethoxy)-4-[2-[(2,6-dimethylpyrimidin-4-yl)amino]pyrazolo[1,5-a]pyridin-5-yl]-3-pyridyl]oxy]-2,2-dimethyl-propanenitrile;
3-[[4-[2-[(2,6-dimethylpyrimidin-4-yl)amino]pyrazolo[1,5-a]pyridin-5-yl]-6-(2-fluoroethoxy)-3-pyridyl]oxy]-2,2-dimethyl-propanenitrile;
3-[[6-hydroxy-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile;
3-[[6-chloro-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile;
3-[[6-(difluoromethoxy)-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile;
(1r,4r)-4-[[6-(difluoromethoxy)-4-[2-[(2,6-dimethylpyrimidin-4-yl)amino]pyrazolo[1,5-a]pyridin-5-yl]-3-pyridyl]oxy]cyclohexanol;
3-[[4-[2-[(2,6-dimethylpyrimidin-4-yl)amino]pyrazolo[1,5-a]pyridin-5-yl]-6-(2-hydroxyethoxy)-3-pyridyl]oxy]-2,2-dimethyl-propanenitrile;
1-[[6-(difluoromethoxy)-4-[2-[(2,6-dimethylpyrimidin-4-yl)amino]pyrazolo[1,5-a]pyridin-5-yl]-3-pyridyl]oxy]-2-methyl-propan-2-ol;
(*R)-3-[[6-(1-cyanoethoxy)-4-[2-[(2,6-dimethylpyrimidin-4-yl)amino]pyrazolo[1,5-a]pyridin-5-yl]-3-pyridyl]oxy]-2,2-dimethyl-propanenitrile;
(*S)-3-[[6-(1-cyanoethoxy)-4-[2-[(2,6-dimethylpyrimidin-4-yl)amino]pyrazolo[1,5-a]pyridin-5-yl]-3-pyridyl]oxy]-2,2-dimethyl-propanenitrile;
3-[[4-[2-[(2-amino-6-methyl-pyrimidin-4-yl)amino]pyrazolo[1,5-a]pyridin-5-yl]-6-(difluoromethoxy)-3-pyridyl]oxy]-2,2-dimethyl-propanenitrile;
3-[[6-(difluoromethoxy)-4-[2-[(6-methylpyrazin-2-yl)amino]pyrazolo[1,5-a]pyridin-5-yl]-3-pyridyl]oxy]-2,2-dimethyl-propanenitrile;
3-[[6-(difluoromethoxy)-4-[2-[(1,5-dimethyl-6-oxo-pyridazin-3-yl)amino]pyrazolo[1,5-a]pyridin-5-yl]-3-pyridyl]oxy]-2,2-dimethyl-propanenitrile;
3-[[6-(difluoromethoxy)-4-[2-[(6-methylpyrimidin-4-yl)amino]pyrazolo[1,5-a]pyridin-5-yl]-3-pyridyl]oxy]-2,2-dimethyl-propanenitrile;
3-[[4-[2-[(2,6-dimethylpyrimidin-4-yl)amino]pyrazolo[1,5-a]pyridin-5-yl]-6-methyl-3-pyridyl]oxymethyl]cyclobutanecarbonitrile;
3-[[4-[2-[(2,6-dimethylpyrimidin-4-yl)amino]pyrazolo[1,5-a]pyridin-5-yl]-6-methyl-3-pyridyl]oxymethyl]cyclobutanecarbonitrile;
3-[[6-(2,2-difluoroethoxy)-4-[2-(pyridazin-3-ylamino)pyrazolo[1,5-a]pyridin-5-yl]-3-pyridyl]oxy]-2,2-dimethyl-propanenitrile;
3-[[6-(2,2-difluoroethoxy)-4-[2-(pyrazin-2-ylamino)pyrazolo[1,5-a]pyridin-5-yl]-3-pyridyl]oxy]-2,2-dimethyl-propanenitrile;
2-[[4-[2-[(2,6-dimethylpyrimidin-4-yl)amino]pyrazolo[1,5-a]pyridin-5-yl]-5-(4-hydroxycyclohexoxy)-2-pyridyl]oxy]acetonitrile;
3-[[4-[2-[(6-aminopyrazin-2-yl)amino]pyrazolo[1,5-a]pyridin-5-yl]-6-(difluoromethoxy)-3-pyridyl]oxy]-2,2-dimethyl-propanenitrile;
3-[[6-(difluoromethoxy)-4-[2-[(1-methylpyrazol-4-yl)amino]pyrazolo[1,5-a]pyridin-5-yl]-3-pyridyl]oxy]-2,2-dimethyl-propanenitrile;
3-[[6-(2,2-difluoroethoxy)-4-[2-[(1,5-dimethyl-6-oxo-pyridazin-3-yl)amino]pyrazolo[1,5-a]pyridin-5-yl]-3-pyridyl]oxy]-2,2-dimethyl-propanenitrile;
3-[[6-(difluoromethoxy)-4-[2-(3-pyridylamino)pyrazolo[1,5-a]pyridin-5-yl]-3-pyridyl]oxy]-2,2-dimethyl-propanenitrile;
3-[[6-(difluoromethoxy)-4-[2-[[5-(morpholinomethyl)-2-pyridyl]amino]pyrazolo[1,5-a]pyridin-5-yl]-3-pyridyl]oxy]-2,2-dimethyl-propanenitrile;
3-[[6-(2,2-difluoroethoxy)-4-[2-[(6-methylpyrazin-2-yl)amino]pyrazolo[1,5-a]pyridin-5-yl]-3-pyridyl]oxy]-2,2-dimethyl-propanenitrile;
3-[[6-(2,2-difluoroethoxy)-4-[2-[(1-methyl-6-oxo-pyridazin-3-yl)amino]pyrazolo[1,5-a]pyridin-5-yl]-3-pyridyl]oxy]-2,2-dimethyl-propanenitrile;
3-[[6-(difluoromethoxy)-4-[2-[(4-methoxypyrimidin-2-yl)amino]pyrazolo[1,5-a]pyridin-5-yl]-3-pyridyl]oxy]-2,2-dimethyl-propanenitrile;
3-[[6-(difluoromethoxy)-4-[2-[(1-methyl-1,2,4-triazol-3-yl)amino]pyrazolo[1,5-a]pyridin-5-yl]-3-pyridyl]oxy]-2,2-dimethyl-propanenitrile;
3-[[6-(difluoromethoxy)-4-[2-[[2-(ethylamino)-6-(trifluoromethyl)pyrimidin-4-yl]amino]pyrazolo[1,5-a]pyridin-5-yl]-3-pyridyl]oxy]-2,2-dimethyl-propanenitrile;
2-[[5-[5-(2-cyano-2-methyl-propoxy)-2-(difluoromethoxy)-4-pyridyl]pyrazolo[1,5-a]pyridin-2-yl]amino]-N-(2-hydroxy-2-methyl-propyl)-6-methyl-pyridine-4-carboxamide;
3-[[6-(difluoromethoxy)-4-[2-[(1-methyl-6-oxo-pyridazin-3-yl)amino]pyrazolo[1,5-a]pyridin-5-yl]-3-pyridyl]oxy]-2,2-dimethyl-propanenitrile;
3-[[4-[2-[(2-tert-butyl-6-methyl-pyrimidin-4-yl)amino]pyrazolo[1,5-a]pyridin-5-yl]-6-(difluoromethoxy)-3-pyridyl]oxy]-2,2-dimethyl-propanenitrile;
3-[[4-[2-[(5-aminopyridazin-3-yl)amino]pyrazolo[1,5-a]pyridin-5-yl]-6-(difluoromethoxy)-3-pyridyl]oxy]-2,2-dimethyl-propanenitrile;
3-[[4-[2-[(2-aminopyrimidin-4-yl)amino]pyrazolo[1,5-a]pyridin-5-yl]-6-(difluoromethoxy)-3-pyridyl]oxy]-2,2-dimethyl-propanenitrile;
3-[[6-(difluoromethoxy)-4-[2-(4,5,6,7-tetrahydropyrazolo[1,5-a]pyrazin-2-ylamino)pyrazolo[1,5-a]pyridin-5-yl]-3-pyridyl]oxy]-2,2-dimethyl-propanenitrile;
3-[[6-(difluoromethoxy)-4-[2-(pyrazin-2-ylamino)pyrazolo[1,5-a]pyridin-5-yl]-3-pyridyl]oxy]-2,2-dimethyl-propanenitrile;
3-[[6-(difluoromethoxy)-4-[2-(pyridazin-3-ylamino)pyrazolo[1,5-a]pyridin-5-yl]-3-pyridyl]oxy]-2,2-dimethyl-propanenitrile;
3-[[6-(difluoromethoxy)-4-[2-[(5-methylpyrazin-2-yl)amino]pyrazolo[1,5-a]pyridin-5-yl]-3-pyridyl]oxy]-2,2-dimethyl-propanenitrile;
3-[[6-(difluoromethoxy)-4-[2-[(6-methyl-2-morpholino-pyrimidin-4-yl)amino]pyrazolo[1,5-a]pyridin-5-yl]-3-pyridyl]oxy]-2,2-dimethyl-propanenitrile;
3-[[6-(difluoromethoxy)-4-[2-[(6-methylpyridazin-3-yl)amino]pyrazolo[1,5-a]pyridin-5-yl]-3-pyridyl]oxy]-2,2-dimethyl-propanenitrile;
3-[[6-(difluoromethoxy)-4-[2-[[6-(1-hydroxy-1-methyl-ethyl)pyridazin-3-yl]amino]pyrazolo[1,5-a]pyridin-5-yl]-3-pyridyl]oxy]-2,2-dimethyl-propanenitrile;
3-[[6-(difluoromethoxy)-4-[2-[[5-(1-hydroxy-1-methyl-ethyl)pyrazin-2-yl]amino]pyrazolo[1,5-a]pyridin-5-yl]-3-pyridyl]oxy]-2,2-dimethyl-propanenitrile;
3-[[6-(difluoromethoxy)-4-[2-[(1-methylpyrazol-3-yl)amino]pyrazolo[1,5-a]pyridin-5-yl]-3-pyridyl]oxy]-2,2-dimethyl-propanenitrile;
2-[[5-[5-(2-cyano-2-methyl-propoxy)-2-(difluoromethoxy)-4-pyridyl]pyrazolo[1,5-a]pyridin-2-yl]amino]-N,6-dimethyl-pyridine-4-carboxamide;
3-[[6-(difluoromethoxy)-4-[2-(5,6,7,8-tetrahydro-2,7-naphthyridin-3-ylamino)pyrazolo[1,5-a]pyridin-5-yl]-3-pyridyl]oxy]-2,2-dimethyl-propanenitrile;
3-[[6-(difluoromethoxy)-4-[2-[(2-methylpyrimidin-4-yl)amino]pyrazolo[1,5-a]pyridin-5-yl]-3-pyridyl]oxy]-2,2-dimethyl-propanenitrile;
3-[[6-(difluoromethoxy)-4-[2-(5,6,7,8-tetrahydro-2,6-naphthyridin-3-ylamino)pyrazolo[1,5-a]pyridin-5-yl]-3-pyridyl]oxy]-2,2-dimethyl-propanenitrile;
3-[[6-(difluoromethoxy)-4-[2-(5,6,7,8-tetrahydro-1,7-naphthyridin-2-ylamino)pyrazolo[1,5-a]pyridin-5-yl]-3-pyridyl]oxy]-2,2-dimethyl-propanenitrile;
1-[[6-(difluoromethoxy)-4-[2-[(2,6-dimethylpyrimidin-4-yl)amino]pyrazolo[1,5-a]pyridin-5-yl]-3-pyridyl]oxymethyl]cyclopropanecarbonitrile;
(*R)-2-[[4-[2-[(2,6-dimethylpyrimidin-4-yl)amino]pyrazolo[1,5-a]pyridin-5-yl]-5-(4-hydroxycyclohexoxy)-2-pyridyl]oxy]propanenitrile;
3-[2-[[4-[2-[(2,6-dimethylpyrimidin-4-yl)amino]pyrazolo[1,5-a]pyridin-5-yl]-6-methyl-3-pyridyl]oxy]ethyl]oxetane-3-carbonitrile;
N-[5-[2-(difluoromethoxy)-5-(4-hydroxycyclohexoxy)-4-pyridyl]pyrazolo[1,5-a]pyridin-2-yl]cyclopropanecarboxamide;
N-[5-[2-(cyanomethoxy)-5-(2-cyano-2-methyl-propoxy)-4-pyridyl]pyrazolo[1,5-a]pyridin-2-yl]cyclopropanecarboxamide;
3-[[6-(2,2-difluoroethoxy)-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile;
(*S)—N-[5-[2-(1-cyanoethoxy)-5-(2-cyano-2-methyl-propoxy)-4-pyridyl]pyrazolo[1,5-a]pyridin-2-yl]cyclopropanecarboxamide;
3-[[6-(difluoromethoxy)-4-[2-[(2,6-dimethylpyrimidin-4-yl)amino]pyrazolo[1,5-a]pyridin-5-yl]-3-pyridyl]oxy]-2,2-dimethyl-propanamide;
3-[[6-(difluoromethoxy)-4-[2-[(2,6-dimethylpyrimidin-4-yl)amino]pyrazolo[1,5-a]pyridin-5-yl]-3-pyridyl]oxy]-N,N,2,2-tetramethyl-propanamide;
3-[[6-(difluoromethoxy)-4-[2-[(2,6-dimethylpyrimidin-4-yl)amino]imidazo[1,2-a]pyrazin-6-yl]-3-pyridyl]oxy]-2,2-dimethyl-propanenitrile;
3-((4-(2-((1-cyclopropyl-5-methyl-6-oxo-1,6-dihydropyridazin-3-yl)amino)pyrazolo[1,5-a]pyridin-5-yl)-6-(2,2-difluoroethoxy)pyridin-3-yl)oxy)-2,2-dimethylpropanenitrile;
(*R)-4-[[6-(difluoromethoxy)-4-[2-[(2,6-dimethylpyrimidin-4-yl)amino]pyrazolo[1,5-a]pyridin-5-yl]-3-pyridyl]oxymethyl]-5,5-dimethyl-pyrrolidin-2-one;
(*S)-4-[[6-(difluoromethoxy)-4-[2-[(2,6-dimethylpyrimidin-4-yl)amino]pyrazolo[1,5-a]pyridin-5-yl]-3-pyridyl]oxymethyl]-5,5-dimethyl-pyrrolidin-2-one;
5-[2-(difluoromethoxy)-5-(2-methyl-2-methyl sulfonyl-propoxy)-4-pyridyl]-N-(2,6-dimethylpyrimidin-4-yl)pyrazolo[1,5-a]pyridin-2-amine;
(*S)-4-[[6-(difluoromethoxy)-4-[2-[(2,6-dimethylpyrimidin-4-yl)amino]pyrazolo[1,5-a]pyridin-5-yl]-3-pyridyl]oxymethyl]-1,5,5-trimethyl-pyrrolidin-2-one;
N-[5-(2-methoxyphenyl)pyrazolo[1,5-a]pyridin-2-yl]cyclopropanecarboxamide;
N-[5-(2-ethoxyphenyl)pyrazolo[1,5-a]pyridin-2-yl]cyclopropanecarboxamide;
N-[5-(2-methoxy-5-methyl-phenyl)pyrazolo[1,5-a]pyridin-2-yl]cyclopropanecarboxamide;
N-[5-(2-methoxy-4-methyl-phenyl)pyrazolo[1,5-a]pyridin-2-yl]cyclopropanecarboxamide;
N-[5-(2-methoxy-6-methyl-phenyl)pyrazolo[1,5-a]pyridin-2-yl]cyclopropanecarboxamide;
N-[5-(2-methoxy-3-methyl-phenyl)pyrazolo[1,5-a]pyridin-2-yl]cyclopropanecarboxamide;
N-[5-(2-methoxy-3-pyridyl)pyrazolo[1,5-a]pyridin-2-yl]cyclopropanecarboxamide;
N-[5-(3-methoxy-4-pyridyl)pyrazolo[1,5-a]pyridin-2-yl]cyclopropanecarboxamide;
N-[5-(3-methoxy-2-pyridyl)pyrazolo[1,5-a]pyridin-2-yl]cyclopropanecarboxamide;
N-[5-(4-methoxy-3-pyridyl)pyrazolo[1,5-a]pyridin-2-yl]cyclopropanecarboxamide;
N-[5-(5-ethoxy-2-methyl-4-pyridyl)pyrazolo[1,5-a]pyridin-2-yl]cyclopropanecarboxamide;
N-[5-(5-benzyloxy-2-cyano-4-pyridyl)pyrazolo[1,5-a]pyridin-2-yl]cyclopropanecarboxamide;
3-[2-[2-[(2,6-dimethylpyrimidin-4-yl)amino]pyrazolo[1,5-a]pyridin-5-yl]-4-(trifluoromethoxy)phenoxy]-2,2-dimethyl-propanenitrile;
(*R)-5-[5-(2-amino-3,3,3-trifluoro-2-methyl-propoxy)-2-(difluoromethoxy)-4-pyridyl]-N-(2,6-dimethylpyrimidin-4-yl)pyrazolo[1,5-a]pyridin-2-amine; and
(*S)-5-[5-(2-amino-3,3,3-trifluoro-2-methyl-propoxy)-2-(difluoromethoxy)-4-pyridyl]-N-(2,6-dimethylpyrimidin-4-yl)pyrazolo[1,5-a]pyridin-2-amine;
   or a tautomer, stereoisomer pharmaceutically acceptable salt, or solvate thereof.

### Methods of Preparation

Compounds described herein can be prepared by any number of processes as described generally below and more specifically illustrated by the exemplary compounds which follow in the Examples section herein. The compounds provided herein as prepared in the processes described below can be synthesized in the form of mixtures of stereoisomers (e.g., enantiomers, diastereomers), including racemic mixtures of enantiomers, that can be separated from one another using art-known resolution procedures, for instance including liquid chromatography using a chiral stationary phase. Additionally or alternatively, stereochemically pure isomeric forms of the compounds described herein can be derived from the corresponding stereochemically pure isomeric forms of the appropriate starting materials, intermediates, or reagents. For example, if a specific stereoisomer is desired, the compound can be synthesized by stereospecific methods of preparation, which typically employ stereochemically pure starting materials or intermediate compounds.

Pharmaceutically acceptable salts of compounds of the application can be synthesized from the parent compound containing an acidic or basic moiety by conventional chemical methods. Generally, such salts can be prepared by reacting the free acid or base forms of these compounds with a stoichiometric amount of the appropriate acid or base in water or in an organic solvent, or in a mixture of the two. Examples of suitable organic solvents include, but are not limited to, ether, ethyl acetate (EtOAc), ethanol, isopropanol, or acetonitrile.

By way of illustration, but not as a limitation, compounds of formula (I) described herein can be prepared according to the following general preparation procedures shown in Schemes 1 and 2. One of ordinary skill in the art will recognize that, to obtain various compounds of formula (I) as described herein, starting materials can be suitably selected so that the ultimately desired substituent groups will be carried through (i.e., be stable over the course of the synthesis) the reaction scheme with or without protection as appropriate to yield the desired product. Alternatively, it may be necessary or desirable to employ, in place of the ultimately desired substituent, a suitable group that may be carried through (i.e., be stable over the course of the synthesis) the reaction scheme and replaced as appropriate with the desired substituent.

Unless otherwise specified, the variables in Schemes 1-2 are as defined above in reference to the various embodiments of compounds of formula (I). The following general features concerning reaction temperatures, protecting groups (PGs), leaving groups (LGs), and substituents R₁, R₂, R₃, R₄, R₅ etc. refer, as applicable, to Schemes 1-2.

If no temperature or temperature range is stated, it is to be understood that the reaction is to be conducted at room temperature.

The term PG in the following Schemes 1-2 represents a protecting group. Examples of protecting groups suitable for use include, but are not limited to, acetyl (Ac), t-butylcarbamate (Boc), and t-butyldimethylsilyl (TBS).

The term LG in the following Schemes 1-2 represents a leaving group. Examples of leaving groups suitable for use include, but are not limited to, halo (e.g., I, Cl, Br, F), mesylate (—OMs), triflate (—OTf), tosylate (—OTs), boronic acid, and boronate ester.

When isomerically pure samples are desired, isomeric mixtures of compounds synthesized according to Scheme 1-2 can be separated by chiral supercritical fluid chromatography (SFC) or high performance liquid chromatography (HPLC).

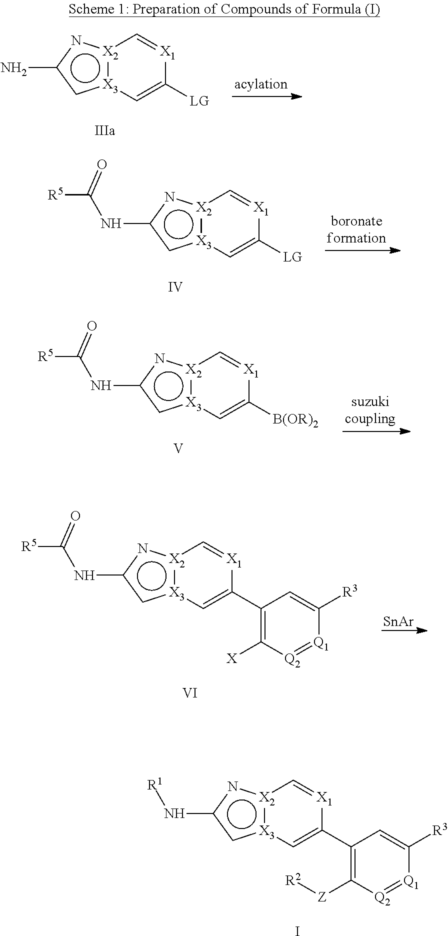

As shown in Scheme 1, a compound of formula (IV) is prepared from a compound of formula (IIIa) by reaction with an acid or activated acid derivative, under suitable amide bond forming conditions, wherein LG is, e.g., Br, Cl, OTf, or I. For example, reaction of an amine of formula (IIIa), wherein LG is Br, Cl, OTf, or I, and an acid or activated acid derivative, with an optional appropriate activating reagent, for example a carbodiimide, such as N,N′-dicyclohexylcarbodiimide (DCC) or 1-ethyl-3-(3-dimethylaminopropyl)carbodiimide (EDCI) optionally in the presence of hydroxybenzotriazole (HOBt) and/or a catalyst such as 4-Dimethylaminopyridine (DMAP); a halotrisaminophosphonium salt such as (Benzotriazol-1-yloxy)tris(dimethylamino)phosphonium hexafluorophosphate (BOP) or Bromotripyrrolidinophosphonium hexafluorophosphate (PyBroP); a suitable pyridinium salt such as 2-chloro-1-methyl pyridinium chloride; or another suitable coupling agent such as 3-[Bis(dimethylamino)methyliumyl]-3H-benzotriazol-1-oxide hexafluorophosphate (HBTU), 1-[Bis(dimethylamino)methylene]-1H-1,2,3-triazolo[4,5-b]pyridinium 3-oxide hexafluorophosphate (HATU), and the like. Coupling reactions are conducted in a suitable solvent such as dichloromethane (DCM), tetrahydrofuran (THF), dimethylformamide (DMF) and the like, optionally in the presence of a tertiary amine such as N-methylmorpholine, N-ethyldiisopropylamine, or triethylamine (TEA), at a temperature ranging from about 0° C. to room temperature, to provide a compound of formula (IV).

A compound of formula (V) is prepared from a compound of formula (IV) under suitable boronate forming conditions. For example, a compound of formula (IV) is treated with a borylating agent such as bis(pinacolato)diboron, in the presence of a palladium catalyst such as Pd(dppf)Cl₂, and the like, and a suitable base, such as potassium acetate, and the like, employing conventional heating, at a temperature such as 100° C., in a solvent such as 1,4-dioxane, and the like, to provide a compound of formula (V).

A compound of formula (VI) is prepared from a compound of formula (V) by a metal-mediated cross coupling reaction. For example, a compound of formula (V) is reacted with a suitably substituted commercially available or synthetically accessible aryl boronic acid, boronate ester, and the like, in the presence of a palladium catalyst such as PdCl₂(dtbpf), Pd(PPh₃)₄, PdCl₂(dppf), Pd(PPh₃)₂Cl₂, Pd(OAc)₂, and the like, with or without the addition of a ligand such as 1,1′-Bis(diphenylphosphino)ferrocene (DPPF); a base such as K₃PO₄, K₂CO₃, aq. Na₂CO₃, Na₂CO₃, Cs₂CO₃, and the like; in a suitable solvent such as 1,2-dimethoxyethane, 1,4-dioxane, DMF, water, or a mixture thereof; at a temperature ranging from 60 to 180° C., employing microwave or conventional heating; for a period of about 30 min to 16 hours, to provide a compound of Formula (VI), wherein X is halo, e.g., Cl or F.

A compound of formula (VI) is reacted with a suitable nucleophile such as an alcohol with or without a suitable base such as TEA, Cs₂CO₃, K₂CO₃, or sodium hydride in a suitable solvent such as DMF, THF, dimethylacetamide (DMA), DCM, and the like, for a period of 1-16 hours, in a nucleophilic aromatic substitution reaction (SnAr) to provide a compound of formula (I). In a preferred embodiment, the base is sodium hydride.

Conversion of compounds of formula (I), wherein R¹ contains a protected or unprotected amine, to a compound of formula (I), the amine may be substituted with methyl or alkyl, is achieved with a suitable aldehyde or ketone or similar equivalents such as paraformaldehyde, acetaldehyde and a reducing agent such as NaBH(OAc)₃, NaBH₄, NaCNBH₃ and the like, in a solvent such as THF, DCM, MeOH and the like, for a period of 12-20 hours.

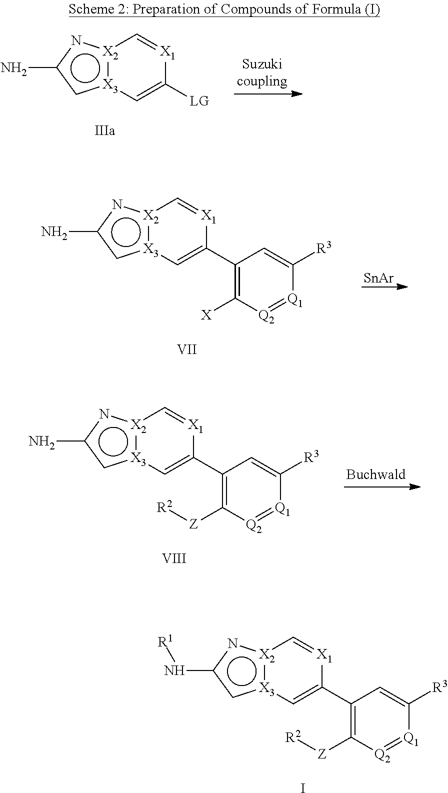

As shown in Scheme 2, a compound of formula (VII) is prepared from a compound of formula (IIIa), wherein LG is, e.g., Br, Cl, F, I, boronic acid or boronate ester, by a metal-mediated cross coupling reaction with a suitably substituted commercially available or synthetically accessible aryl-halide, aryl-boronic acid, aryl boronate ester, and the like, wherein X is halo, e.g., F or Cl. For example, a compound of formula (IIIa), is reacted with a suitably substituted commercially available or synthetically accessible aryl boronic acid, boronate ester, and the like, in the presence of a palladium catalyst such as PdCl₂(dtbpf), Pd(PPh₃)₄, PdCl₂(dppf), Pd(PPh₃)₂Cl₂, Pd(OAc)₂, and the like; with or without the addition of a ligand such as DPPF; a base such as K₃PO₄, K₂CO₃, aq. Na₂CO₃, Na₂CO₃, Cs₂CO₃, and the like; in a suitable solvent such as 1,2-dimethoxyethane, 1,4-dioxane, DMF, dioxane, THF, water, or a mixture thereof; at a temperature ranging from 60 to 180° C., employing microwave or conventional heating; for a period of about 30 min to 16 hours, to provide a compound of Formula (VII).

A compound of formula (VII) is reacted with a suitable nucleophile such as an amine or alcohol with or without a suitable base such as TEA, Cs₂CO₃, K₂CO₃, or sodium hydride in a suitable solvent such as DMF, THF, DMA, DCM, and the like, for a period of 1-16 hours, to provide a compound of formula (VIII) via an aromatic nucleophilic substitution reaction (SnAr).

A compound of formula (VIII) is then reacted under metal-mediated cross coupling conditions to form a compound of formula (I). For example, a compound of formula (VIII) is reacted with a commercially available or synthetically accessible suitably substituted aryl or heteroaryl; in the presence of a palladium catalyst such as RuPhos Pd G3, Pd(Ph₃P)₄, Pd(dppf)Cl₂, PdCl₂(dppf)-CH₂Cl₂, PdCl₂(dtbpf), and the like; a suitable base such a sodium carbonate (Na₂CO₃), potassium phosphate, cesium carbonate (Cs₂CO₃), potassium carbonate (K₂CO₃) and the like; in a solvent such as 1,4-dioxane, water, or a mixture thereof, employing conventional or microwave heating at temperatures ranging from room temperature to 100° C. for a period of 1 h to 18 h, to give a compound of formula (I).

Conversion of compounds of formula (I), where R′ contains a protected or unprotected amine, to a compound of formula (I), wherein amine is substituted with methyl or alkyl, is achieved with a suitable aldehyde or ketone or similar equivalents such as paraformaldehyde, acetaldehyde, a reducing agent such as NaBH(OAc)₃, NaBH₄, NaCNBH₃ and the like, in a solvent such as THF, DCM, MeOH and the like, for a period of 12-20 hours.

### Compositions

In another aspect, provided is a pharmaceutical composition comprising a compound of formula (I), or a tautomer, stereoisomer, pharmaceutically acceptable salt or solvate thereof, as described herein.

Compositions can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions can also be formulated for other routes of administration including transmucosal, ocular, rectal, long acting implantation, sublingual administration, under the tongue, from oral mucosa bypassing the portal circulation, inhalation, or intranasal.

In yet another aspect, provided is a method of preparing a pharmaceutical composition comprising combining a compound of formula (I), or a tautomer, stereoisomer, pharmaceutically acceptable salt or solvate thereof, with at least one pharmaceutically acceptable carrier. Pharmaceutical compositions can be prepared by any method known in the art in view of the present disclosure, and one of ordinary skill in the art will be familiar with such techniques used to prepare pharmaceutical compositions. For example, a pharmaceutical composition according to the application can be prepared by mixing a compound of formula (I) with one or more pharmaceutically acceptable carriers according to conventional pharmaceutical compounding techniques, including but not limited to, conventional admixing, dissolving, granulating, emulsifying, encapsulating, entrapping or lyophilizing processes.

### Methods of Use

In another general aspect, provided are methods of inhibiting a salt inducible kinase (SIK) and methods of treating a disease related to SIKs, including autoimmune disorders and proliferative disorders, using the compounds and compositions, e.g., pharmaceutical compositions, described herein.

As used herein, “an effective amount” means an amount of a composition or compound that elicits a biological or medicinal response in a tissue system or subject that is being sought by a researcher, veterinarian, medical doctor or other conditions, which can include alleviation of the symptoms of the disease, disorder, or condition being treated. An effective amount can vary depending upon a variety of factors, such as the physical condition of the subject, age, weight, health, etc.; and the particular disease, disorder, or condition to be treated. An effective amount can readily be determined by one of ordinary skill in the art in view of the present disclosure.

According to particular embodiments, an effective amount refers to the amount of a composition or compound described herein which is sufficient to inhibit a salt inducible kinase (SIK). In another particular embodiment, an effective amount refers to the amount of a composition or compound described herein which is sufficient to treat a disease, disorder, or condition mediated by a SIK.

In an embodiment, provided is a method of inhibiting a salt inducible kinase (SIK) in a subject in need thereof, comprising administering to the subject a compound or composition described herein, e.g., administering an effective amount of a compound or composition described herein.

In some embodiments, the salt inducible kinase is SIK1.

In some embodiments, the salt inducible kinase is SIK2.

In some embodiments, the salt inducible kinase is SIK3.

In some embodiments, the salt inducible kinase is SIK1 and SIK2.

In some embodiments, the salt inducible kinase is SIK1 and SIK3.

In some embodiments, the salt inducible kinase is SIK2 and SIK3.

In some embodiments, the salt inducible kinase is SIK1, SIK2, and SIK3.

In an embodiment, provided is a method of treating a disease, disorder, or condition mediated by a SIK in a subject in need thereof, comprising administering to the subject a compound or composition described herein, e.g., administering an effective amount of a compound or composition described herein.

In some embodiments, a disease, disorder, or condition is mediated by SIK1.

In some embodiments, a disease, disorder, or condition is mediated by SIK2.

In some embodiments, a disease, disorder, or condition is mediated by SIK3.

In some embodiments, a disease, disorder, or condition is mediated by SIK1 and SIK2.

In some embodiments, a disease, disorder, or condition is mediated by SIK1 and SIK3.

In some embodiments, a disease, disorder, or condition is mediated by SIK2 and SIK3.

In some embodiments, a disease, disorder, or condition is mediated by SIK1, SIK2, and SIK3.

In some embodiments, a disease, disorder, or condition mediated by a SIK is an autoimmune disorder.

In some embodiments, a disease, disorder, or condition mediated by a SIK is a proliferative disorder.

In other general aspects, provided are compounds and compositions described herein for use in methods of inhibiting a salt inducible kinase (SIK) and/or methods of treating a disease, disorder, or condition mediated by a SIK. In yet other general aspects, provided are uses of the compounds and compositions described herein in the manufacture of a medicament for inhibiting a salt inducible kinase (SIK) and/or for treating a disease, disorder, or condition mediated by a SIK.

### EXAMPLES

The following examples of the application are to further illustrate the nature of the application. It should be understood that the following examples do not limit the application and the scope of the application is to be determined by the appended claims.

### Synthesis Examples

### Intermediate 1: 5-bromopyrazolo[1,5-a]pyridin-2-amine

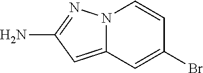 [SMILES: Nc1cc2cc(Br)ccn2n1]

Step A: 2-(4-bromopyridin-2-yl)acetonitrile. To a solution of n-BuLi (1.9 moles) in THF (800 mL, 8 mL/g) cooled to −78° C. was charged acetonitrile (2.1 moles) while maintaining an internal temperature of −78 C±5° C. The resulting mixture was stirred at −78° C. for 45 minutes and then was charged with a solution of 4-bromo-2-fluoropyridine (100 g, 0.57 moles) in THF (200 mL, 2 mL/g) while maintaining an internal temperature of −78±5° C. The reaction mixture was warmed to −30° C. and stirred for 2 hours. Once HPLC analysis indicated complete consumption of all the starting material, the reaction was quenched by the slow addition of saturated aqueous ammonium chloride (100 mL, 1 mL/g) and then warmed to 23° C. The mixture was concentrated and then diluted with water (1000 mL, 10 mL/g) followed by ethyl acetate (EtOAc) (1000 mL, 10 mL/g). The layers were separated and the organic phase was washed with brine (1000 mL, 10 mL/g) and dried with Na₂SO₄. The organic phase was filtered and concentrated in vacuo to give a black oil which was then purified by silica gel chromatography (ether:EtOAc=15:1 to 5:1) to give the title compound as a yellow solid (87 g, 0.44 moles, 77%).

Step B: 1-amino-4-bromo-2-(cyanomethyl)pyridin-1-ium 2,4,6-trimethylbenzenesulfonate. To a solution of O-(mesitylsulfonyl)hydroxylamine (1.8 kg, 8.2 moles) in DCM (9.0 L, 10 L/kg) which had been cooled to 5±5° C. was charged 2-(4-bromopyridin-2-yl)acetonitrile (0.90 kg, 4.6 mols example 727) while maintaining an internal temperature of 5±5° C. The reaction mixture was slowly warmed to 23° C. and stirred for 19 hours. The resulting slurry was then filtered and washed with DCM (1.8 L, 2 L/kg) and dried to give the title compound as an off-white solid (2.15 kg) which was stored in the freezer and used without further purification in the following step.

Step C: 5-bromopyrazolo[1,5-a]pyridin-2-amine. To a solution of 1-amino-4-bromo-2-(cyanomethyl)pyridin-1-ium 2,4,6-trimethylbenzenesulfonate (263 g, 0.638 moles) in MeOH (2630 mL, 10 mL/g) heated at 45° C. was charged K₂CO₃ (341 g, 1.28 moles) and stirred for 3 hours at 45° C. The mixture was then concentrated and the solids slurried in water at 23° C. overnight, filtered and dried to give the title compound (89 g, 0.447 moles, 70%) as a brown solid. MS (ESI): mass calculated (calcd.) for C₇H₆BrN₃, 211.0. m/z found, 212.0 [M+H]⁺.

### Intermediate 2: N-(5-bromopyrazolo[1,5-a]pyridin-2-yl)cyclopropanecarboxamide

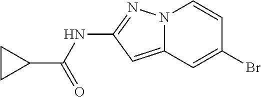 [SMILES: O=C(Nc1cc2cc(Br)ccn2n1)C1CC1]

A vessel containing 5-bromopyrazolo[1,5-a]pyridin-2-amine (Intermediate 1, 407 g, 1.45 mol) and DMA (8150 mL) was cooled at 10° C. Cyclopropanecarbonyl chloride (159 g, 1.53 mol) was added dropwise. The reaction mixture warmed to 20° C. and stirred for 1 h. Water (8 L) was added and the mixture was stirred overnight at room temperature. The resulting solid was isolated by filtration and dried under vacuum to provide the title compound (510 g, 95%) as a tan solid. MS (ESI): mass calcd. for C₁₁H₁₀BrN₃O, 279.00. m/z found, 280.0/282.0 [M+H]⁺.

### Intermediate 3: N-(5-(4,4,5,5-Tetramethyl-1,3,2-dioxaborolan-2-yl)pyrazolo[1,5-a]pyridin-2-yl)cyclopropanecarboxamide

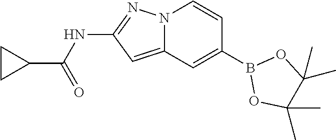 [SMILES: CC1(C)OB(c2ccn3nc(NC(=O)C4CC4)cc3c2)OC1(C)C]

To vessel containing N-(5-bromopyrazolo[1,5-a]pyridin-2-yl)cyclopropanecarboxamide (Intermediate 2, 300 g, 1.08 mol) and potassium acetate (211 g, 2.15 mol) were added dioxane (3 L) and water (19.4 mL, 1.08 mmol). Nitrogen was bubbled through the mixture for 20 min, then bis(pinacolato)diboron (302 g, 1.19 mol) was added at room temperature. The reaction mixture was purged with N₂, heated at 70° C., charged with Pd(dppf)Cl₂ (39.5 g, 0.054 mol) and stirred for 2 h under N₂. The reaction mixture was cooled to rt, filtered, and the resulting solid was rinsed with EtOAc. The resulting solid was purified by column chromatography on silica gel using petroleum ether/ethyl acetate (50:1 to 10:1, gradient elution) to provide the title compound (230 g, 66%). MS (ESI): mass calcd. for C₁₇H₁₇BN₃O₃, 327.18. m/z found, 328.2 [M+H]⁺ and peak for corresponding boronic acid C₁₁H₁₂BN₃O₃ 246.1 [M+H]⁺.

### Intermediate 4: N-(5-(2-Cyano-5-fluoropyridin-4-yl)pyrazolo[1,5-a]pyridin-2-yl)cyclopropanecarboxamide

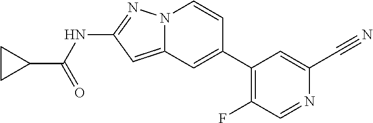 [SMILES: N#Cc1cc(-c2ccn3nc(NC(=O)C4CC4)cc3c2)c(F)cn1]

Step A: N-(5-(2-chloro-5-fluoropyridin-4-yl)pyrazolo[1,5-a]pyridin-2-yl)cyclopropanecarboxamide. To a round bottom flask containing N-(5-(4,4,5,5-tetramethyl-1,3,2-dioxaborolan-2-yl)pyrazolo[1,5-a]pyridin-2-yl)cyclopropanecarboxamide (Intermediate 3, 15.0 g, 45.4 mmol. 1.00 equiv), 2-chloro-5-fluoro-4-iodopyridine (11.7 g, 45.4 mmol, 1.00 equiv), PdCl₂(dppf) (0.90 g, 1.23 mmol, 2.7 mol %), K₃PO₄ (0.5 M in water, 123 mL, 61.5 mmol, 1.35 equiv) and 1,4-dioxane (150 mL, 0.3 M) was evacuated and backfilled with nitrogen (3×). The flask was then placed in a pre-heated heating block at 60° C. After 30 minutes, water (600 mL) was added dropwise at 60° C. The resulting slurry was stirred for 1 hour and then slowly cooled to 23° C. The mixture was then filtered and the solids washed with water (3×40 mL) and then dried in vacuo overnight to give the title compound (15.3 g, 100% yield, 45.4 mmol) as an orange solid. MS (ESI): mass calcd. for C₁₆H₁₂N₄OClF, 330.7. m/z found, 331.1 [M+H]⁺.

Step B: N-(5-(2-cyano-5-fluoropyridin-4-yl)pyrazolo[1,5-a]pyridin-2-yl)cyclopropanecarboxamide. To a 20 mL vial was charged N-(5-(2-chloro-5-fluoropyridin-4-yl)pyrazolo[1,5-a]pyridin-2-yl)cyclopropanecarboxamide (1016 mg, 3.072 mmol and zinc cyanide (331 mg, 2.76 mmol). The vial was capped with a septum and the contents were evacuated and backfilled with argon (2×). A solution of DMF (6 mL) and DMA (2 mL) was purged with argon and then added to the reaction vial. The vial was the heated at 100° C. for 20 min prior to the addition of Pd(PPh₃)₄ (322 mg, 0.279 mmol). The reaction mixture was heated at 90° C. for 95 min. The mixture was cooled to room temperature and added into water (150 mL) with constant stirring resulting in the formation of a precipitate. The solids were collected by suction filtration and washed with water (50 mL). The solids were dried open to air then further dried at 80° C. under high vacuum for 5 h to afford the title compound (1350 mg, 100%) as a tan solid. MS (ESI): mass calcd. for C₁₇H₁₂FN₅O, 321.3. m/z found, 322.1 [M+H]⁺.

### Intermediate 5: N-(5-(5-Chloro-2-cyanopyridin-4-yl)pyrazolo[1,5-a]pyridin-2-yl)cyclopropanecarboxamide

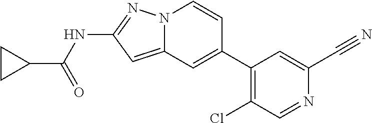 [SMILES: N#Cc1cc(-c2ccn3nc(NC(=O)C4CC4)cc3c2)c(Cl)cn1]

To a 1000 mL round-bottomed flask was charged 4-bromo-5-chloropyridine-2-carbonitrile (15.1 g, 69.3 mmol), N-[5-(4,4,5,5-tetramethyl-1,3,2-dioxaborolan-2-yl)pyrazolo[1,5-a]pyridin-2-yl]cyclopropanecarboxamide (Intermediate 3, 25.0 g, 75.8 mmol), and dichloro(diphenylphosphinoferrocene)palladium (II) (1.60 g, 2.18 mmol). The flask was equipped with a septum then evacuated and backfilled with nitrogen (2×). 1,4-dioxane (231 mL) and 0.5 M potassium phosphate (aq) (183 mL) were added. The resulting mixture was heated at 90° C. for 30 min. The mixture was cooled to room temperature and added into water (1400 mL) with constant stirring resulting in the formation of a precipitate. The solids were collected by suction filtration and washed with water (250 mL). The solids were dried open to air then further dried at 70° C. under high vacuum for 5 h to afford the title compound (26.0 mg, 100%) as an off-white solid. MS (ESI): mass calcd. for C₁₇H₁₂ClN₅O, 337.1. m/z found, 338.1/340.1 [M+H]⁺.

### Intermediate 6: N-(5-(5-Fluoro-2-methylpyridin-4-yl)pyrazolo[1,5-a]pyridin-2-yl)cyclopropanecarboxamide

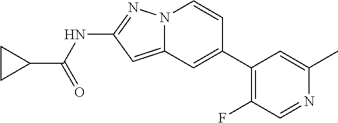 [SMILES: Cc1cc(-c2ccn3nc(NC(=O)C4CC4)cc3c2)c(F)cn1]

To N-(5-(4,4,5,5-tetramethyl-1,3,2-dioxaborolan-2-yl)pyrazolo[1,5-a]pyridin-2-yl)cyclopropanecarboxamide (Intermediate 3, 20.9 g, 64.0 mmol), 4-bromo-5-fluoro-2-methylpyridine (9.82 g, 51.7 mmol), and 1,1′-bis(diphenylphosphino)ferrocene-2-palladium(II)dichloride dichloromethane complex (2.08 g, 2.55 mmol) were added dioxane (200 mL) and 2 M aqueous potassium carbonate (100 mL). The reaction vessel was evacuated with sonication for 2 minutes, purged with N₂, the placed on a pre-heated block at 60° C. for 1 h. The reaction mixture was cooled to room temperature, treated with water (500 mL) and extracted with EtOAc (2×500 mL). The combined organic layers were dried (MgSO₄), filtered and concentrated under reduced pressure. The residue was purified by flash column chromatography (SiO₂, 60-100% EtOAc/DCM) and the fractions containing product were combined and concentrated to afford the title compound (13.7 g, 85%) as a tan solid. MS (ESI): mass calcd. for C₁₇H₁₅FN₄O, 310.1. m/z found, 311.1 [M+H]⁺.

### Intermediate 7: N-(5-(5-Hydroxy-2-methylpyridin-4-yl)pyrazolo[1,5-a]pyridin-2-yl)cyclopropanecarboxamide

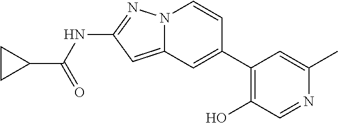 [SMILES: Cc1cc(-c2ccn3nc(NC(=O)C4CC4)cc3c2)c(O)cn1]

N-(5-(4,4,5,5-tetramethyl-1,3,2-dioxaborolan-2-yl)pyrazolo[1,5-a]pyridin-2-yl)cyclopropanecarboxamide (Intermediate 3, 3.50 g, 10.7 mmol), 4-iodo-6-methylpyridin-3-ol hydrochloride (2.56 g, 9.41 mmol), Pd(dppf)Cl₂ (391 mg, 0.535 mmol), and potassium phosphate (9.08 g, 42.8 mmol) was treated with 1,4-dioxane/H₂O (100 mL, 4:1). The reaction vessel was purged with N₂ and placed on a pre-heated block at 90° C. for 2 h. The organic layer was separated, concentrated under reduced pressure and the residue was purified by FCC (eluent: petroleum ether:ethyl acetate=1:0 to 0:1, gradient elution) to afford the title compound (3.06 g, 93%) as a yellow solid. MS (ESI): mass calcd. for C₁₇H₁₆N₄O₂, 308.1. m/z found, 309.0 [M+H]⁺.

### Intermediate 8: 5-(5-Fluoro-2-methylpyridin-4-yl)pyrazolo[1,5-a]pyridin-2-amine

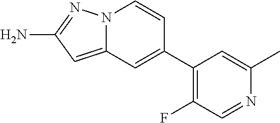 [SMILES: Cc1cc(-c2ccn3nc(N)cc3c2)c(F)cn1]

To 5-bromopyrazolo[1,5-a]pyridin-2-amine (Intermediate 1, 20.0 g, 94.2 mmol), 5-fluoro-2-methyl-4-(4,4,5,5-tetramethyl-1,3,2-dioxaborolan-2-yl)pyridine (32.5 g, 137 mmol), and RuPhos Pd G2 (3.67 g, 4.73 mmol) was added dioxane (200 mL) and 0.5 M tribasic potassium phosphate (300 mL). The reaction mixture was degassed under vacuum with sonication for 2 minutes, purged with N₂ then stirred at 90° C. for 1.5 h. The reaction mixture was cooled to room temperature, treated with saturated aqueous sodium chloride (500 mL) and 10:1 DCM:MeOH (500 mL) then extracted. The organic layer was separated and the aqueous layer was extracted again 10:1 DCM:MeOH (2×500 mL). The combined organic layers were dried (MgSO₄) and concentrated. The residue was purified by flash column chromatography (70-100% ethyl acetate/hexanes) and the fractions containing product were combined and concentrated to afford the title compound (10.9 g, 48%) as a yellow solid. MS (ESI): mass calcd. for C₁₃H₁₁FN₄, 242.1. m/z found, 243.1 [M+H]⁺.

### Intermediate 9: tert-butyl (1R,5S,7s)-7-((4-(2-Aminopyrazolo[1,5-a]pyridin-5-yl)-6-methylpyridin-3-yl)oxy)-3-oxa-9-azabicyclo[3.3.1]nonane-9-carboxylate

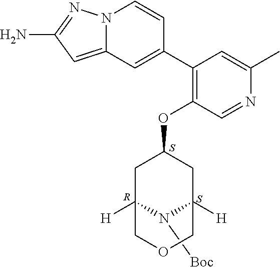

To a round bottom flask containing tert-butyl (1R,5S,7s)-7-hydroxy-3-oxa-9-azabicyclo[3.3.1]nonane-9-carboxylate (4.52 g, 18.6 mmol) and N-methyl-2-pyrrolidone (50.0 mL, 0.25 M) was added sodium hydride (60 wt % dispersion in mineral oil, 4.95 g, 123 mmol) in three portions at 23° C. After 10 minutes, 5-(5-fluoro-2-methylpyridin-4-yl)pyrazolo[1,5-a]pyridin-2-amine (Intermediate 8, 3.00 g, 12.4 mmol) was added in one portion. The resulting mixture was placed in a pre-heated block at 100° C. for 15 minutes and then cooled to 0° C. The reaction mixture was quenched by the slow addition of a solution of ammonium chloride (12.3 g, 247 mmol) in water (50 mL) and then was added 10% MeOH in DCM (100 mL). The layers were separated and the aqueous layer was extracted with 10% MeOH in DCM (2×100 mL). The combined organic layers were washed with 13 wt % aqueous NaCl (2×50 mL) and then dried with MgSO₄ and concentrated in vacuo. The residue was purified by flash column chromatography (1-10% MeOH in DCM) and the fractions containing product were combined and concentrated to afford the title compound (1.30 g, 22%) as an oil. MS (ESI): mass calcd. for C₂₅H₃₁N₅O₄, 465.5. m/z found, 466.3 [M+H]⁺.

### Intermediate 10: tert-butyl (1R,5S,7r)-7-((6-Chloro-4-(2-(cyclopropanecarboxamido)pyrazolo[1,5-a]pyridin-5-yl)pyridin-3-yl)oxy)-3-oxa-9-azabicyclo[3.3.1]nonane-9-carboxylate

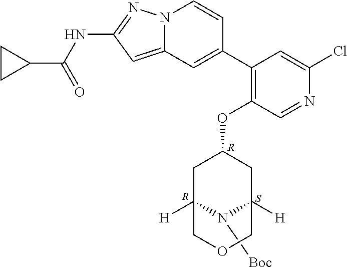

Step A: To tert-butyl (1R,5S,7s)-7-hydroxy-3-oxa-9-azabicyclo[3.3.1]nonane-9-carboxylate (1.00 g, 4.11 mmol), 6-chloro-4-iodopyridin-3-ol (1.05 g, 4.11 mmol), di-tert-butyl-azodicarboxylate (1.23 g, 5.34 mmol) in THF (30 mL, 0.10 M) was added triphenylphosphine (1.40 g, 5.34 mmol) in one portion at 23° C. After 30 minutes, di-tert-butyl-azodicarboxylate (0.60 g, 2.60 mmol) was added followed by triphenylphosphine (0.70 g, 2.60 mmol) at 23° C. After 30 minutes, the reaction mixture was concentrated in vacuo and the residue was purified by flash column chromatography (0-30% EtOAc in hexanes) and the fractions containing product were combined and concentrated to afford tert-butyl (1R,5S,7r)-7-((6-chloro-4-iodopyridin-3-yl)oxy)-3-oxa-9-azabicyclo[3.3.1]nonane-9-carboxylate (1.50 g, 49%) as a white solid. MS (ESI): mass calcd. for C₁₇H₂₂N₂O₄Cl, 480.7. m/z found, 425.0 [[M-tBu]+H]⁺.

Step B: A mixture of N-(5-(4,4,5,5-tetramethyl-1,3,2-dioxaborolan-2-yl)pyrazolo[1,5-a]pyridin-2-yl)cyclopropanecarboxamide (Intermediate 3, 1.20 g, 3.63 mmol), tert-butyl (1R,5S,7r)-7-((6-chloro-4-iodopyridin-3-yl)oxy)-3-oxa-9-azabicyclo[3.3.1]nonane-9-carboxylate (2.64 g, 3.63 mmol), PdCl₂(dppf) (0.072 g, 0.099 mmol, 2.7 mol %), K₃PO₄ (0.5 M in water, 9.9 mL, 4.92 mmol) and 1,4-dioxane (12.1 mL, 0.3 M) was evacuated and backfilled with nitrogen (3×). The flask was then placed in a pre-heated heating block at 60° C. After 1 hour, the solution was cooled to 23° C. and was added EtOAc (30 mL) followed by water (30 mL). The layers were separated and the aqueous layer was extracted with EtOAc (2×20 mL) and the combined organics were dried with MgSO₄, filtered and concentrated in vacuo. The residue was purified by flash column chromatography (10-100% EtOAc in hexanes) to afford the title compound (1.33 g, 66%) as a beige foam. MS (ESI): mass calcd. for C₂₈H₃₂N₅O₅Cl, 554.0. m/z found, 576.3 [M+Na]⁺.

### Intermediate 11: N-(5-(5-Fluoro-2-(prop-1-yn-1-yl)pyridin-4-yl)pyrazolo[1,5-a]pyridin-2-yl)cyclopropanecarboxamide

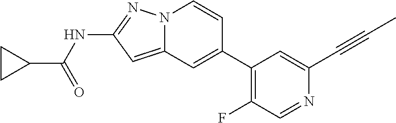 [SMILES: CC#Cc1cc(-c2ccn3nc(NC(=O)C4CC4)cc3c2)c(F)cn1]

Step A: A mixture of N-(5-(4,4,5,5-tetramethyl-1,3,2-dioxaborolan-2-yl)pyrazolo[1,5-a]pyridin-2-yl)cyclopropanecarboxamide (Intermediate 3, 2.00 g, 6.05 mmol), 2-chloro-5-fluoro-4-iodopyridine (1.56 g, 6.05 mmol), PdCl₂(dppf) (0.12 g, 0.164 mmol, 2.7 mol %), K₃PO₄ (0.5 M in water, 16.4 mL, 8.20 mmol) and 1,4-dioxane (20.0 mL, 0.3 M) was evacuated and backfilled with nitrogen (3×). The flask was then placed in a pre-heated heating block at 60° C. After 30 minutes, water (100 mL) was added dropwise at 60° C. The resulting slurry was stirred for 1 hour and then slowly cooled to 23° C. The mixture was then filtered and the solids washed with water (3×6.0 mL) and then dried in vacuo overnight to give N-(5-(2-chloro-5-fluoropyridin-4-yl)pyrazolo[1,5-a]pyridin-2-yl)cyclopropanecarboxamide (2.01 g, 100%) as an orange solid. MS (ESI): mass calcd. for C₁₆H₁₂N₄OClF, 330.7. m/z found, 331.1 [M+H]⁺.

Step B: To a round bottom flask containing N-(5-(2-chloro-5-fluoropyridin-4-yl)pyrazolo[1,5-a]pyridin-2-yl)cyclopropanecarboxamide (0.50 g, 1.51 mmol), 1,1′-Bis(diphenylphosphino)ferrocene-palladium(II)dichloride dichloromethane complex (0.061 g, 0.076 mmol, 5 mol %) and 1,4-dioxane (5.0 mL, 0.3 M) was sparged with Argon for 10 minutes and then was added tributyl(1-propynyl)tin (1.00 g, 3.02 mmol) via syringe under nitrogen. The reaction vessel was placed in a pre-heated heating block at 70° C. After 40 hrs, the mixture was cooled to 23° C. and concentrated in vacuo. The residue was purified by flash column chromatography (0-60% EtOAc in hexanes) and the fractions containing product were combined and concentrated to afford the title compound (0.40 g, 78%) as a pale yellow solid. MS (ESI): mass calcd. for C₁₉H₁₅N₄OF, 334.4. m/z found, 335.1 [M+H]⁺.

### Intermediate 12: (1r,4r)-4-((4-(2-aminopyrazolo[1,5-a]pyridin-5-yl)-6-methylpyridin-3-yl)oxy)cyclohexan-1-ol

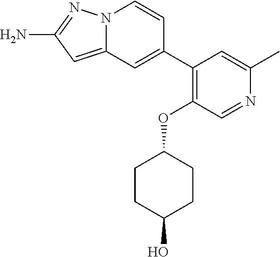 [SMILES: Cc1cc(-c2ccn3nc(N)cc3c2)c(O[C@H]2CC[C@H](O)CC2)cn1]

To a flask containing trans-cyclohexane-1,4-diol (1.61 g, 13.9 mmol) and dimethylacetamide (16.5 mL, 0.25 M) was added sodium hydride (60 wt % dispersion in mineral oil, 0.83 g, 20.6 mmol) in three portions at 23° C. After 10 minutes, 5-(5-fluoro-2-methylpyridin-4-yl)pyrazolo[1,5-a]pyridin-2-amine (Intermediate 8, 1.00 g, 4.13 mmol) was added in one portion and then the reaction was placed in a pre-heated heating block at 80° C. After 5 hours, the reaction mixture was cooled to 23° C. and then diluted with DCM (30 mL) and then slowly quenched with saturated aq. NH₄Cl (20 mL) and then further diluted with water (20 mL). The layers were separated and the organic layer was washed with sat. aq. NaCl (2×20 mL), dried with MgSO₄, filtered and concentrated in vacuo. The residue was purified by flash column chromatography (1-5% MeOH in DCM) and the fractions containing product were combined and concentrated to afford the title compound (0.53 g, 35%) as an oil. MS (ESI): mass calcd. for C₁₉H₂₂N₄O₂, 338.4. m/z found, 339.1 [M+H]⁺.

### Intermediate 13: tert-butyl (S)-2-(((4-(2-aminopyrazolo[1,5-a]pyridin-5-yl)-6-methylpyridin-3-yl)oxy)methyl)morpholine-4-carboxylate

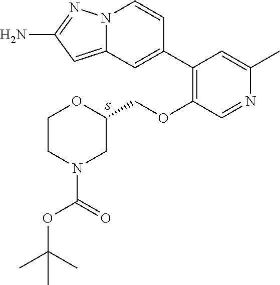

The title compound (530 mg, 42%) was prepared as described in Intermediate 9 where (S)—N-Boc-2-hydroxymethylmorpholine was used instead of (1R,5S,7s)-7-hydroxy-3-oxa-9-azabicyclo[3.3.1]nonane-9-carboxylate. ¹H NMR (400 MHz, DMSO-d₆) δ 8.32-8.28 (m, 2H), 7.58 (d, J=0.8 Hz, 1H), 7.32 (s, 1H), 6.80 (dd, J=1.6, 7.2 Hz, 1H), 5.70 (s, 1H), 5.34 (s, 2H), 4.16 (d, J=4.8 Hz, 2H), 3.92-3.81 (m, 2H), 3.77-3.63 (m, 3H), 3.45-3.42 (m, 2H), 2.45 (s, 3H), 1.36 (s, 9H). MS (ESI): mass calcd. for C₂₃H₂₉N₅O₄, 439.2. m/z found, 440.2 [M+H]⁺

### Intermediate 14: 5-(4,4,5,5-tetramethyl-1,3,2-dioxaborolan-2-yl)pyrazolo[1,5-a]pyridin-2-amine

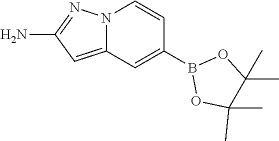 [SMILES: CC1(C)OB(c2ccn3nc(N)cc3c2)OC1(C)C]

To a 200 mL round-bottom flask was charged Intermediate 1 (5.10 g, 24.0 mmol), bis(pinacolato)diboron (12.5 g, 48.1 mmol, 2 equiv), potassium acetate (7.08 g, 72.2 mmol, 3 equiv), 1,1′-bis(diphenylphosphino)ferrocene-palladium(ii)dichloride dichloromethane complex (1.98 g, 2.41 mmol, 0.1 equiv), and 1,4-dioxane (96 mL). The flask was capped with septum then evacuated and backfilled with N₂ several times. The flask was heated to 80° C. for 18 h. The rxn mixture was simply concentrated to dryness and purified directly by column chromatography on silica gel using hexanes/ethyl acetate (1:0 to 0:1; gradient elution) to provide the title compound (4.83 g, 77%). ¹H NMR (400 MHz, DMSO-d₆) δ 8.24-8.16 (m, 1H), 7.60 (t, J=1.2 Hz, 1H), 6.63 (dd, J=6.8, 1.4 Hz, 1H), 5.73-5.67 (m, 1H), 5.31 (s, 2H), 1.30 (s, 12H). MS (ESI): mass calcd. for C₁₃H₁₈BN₃O₂, 259.1. m/z found, 178.1 [M+H]⁺ peak for corresponding boronic acid C₇H₈BN₃O₂ 177.0 [M+H]⁺.

### Intermediate 15: N-(2,6-dimethylpyrimidin-4-yl)-5-(5-fluoro-2-methylpyridin-4-yl)pyrazolo[1,5-a]pyridin-2-amine

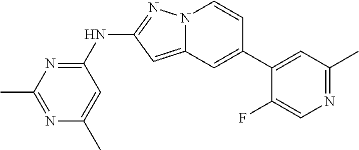

A mixture consisting of Intermediate 8 (7.97 g, 29.0 mmol), 4-chloro-2,6-dimethylpyrimidine (4.1 g, 29 mmol), Brettphos-Pd-G3 (2.6 g, 2.9 mmol), Brettphos (3.1 g, 5.8 mmol), Cs₂CO₃ (28 g, 87 mmol) and 1,4-dioxane (150 mL) was purged with N₂ for 2 min. The resultant mixture was stirred at 50° C. for 6 h before cooling to room-temperature. The reaction mixture was concentrated to dryness under reduced pressure to give the crude product, which was purified by FCC (eluent: petroleum ether:ethyl acetate)=100/0 to 0/100) to yield the title compound (8 g, 79%) as a yellow solid. ¹H NMR (400 MHz, DMSO-d₆) δ 10.29 (s, 1H), 8.67 (d, J=7.2 Hz, 1H), 8.53 (d, J=2.6 Hz, 1H), 7.93 (s, 1H), 7.61 (d, J=6.4 Hz, 1H), 7.07-7.02 (m, 1H), 7.00 (s, 1H), 6.96 (s, 1H), 2.53 (s, 3H), 2.47 (s, 3H), 2.30 (s, 3H). MS (ESI): mass calcd. for C₁₉H₁₇FN₆, 348.4. m/z found, 349.2 [M+H]⁺

### Intermediate 16: 4-(2-((2,6-dimethylpyrimidin-4-yl)amino)pyrazolo[1,5-a]pyridin-5-yl)-6-methylpyridin-3-ol

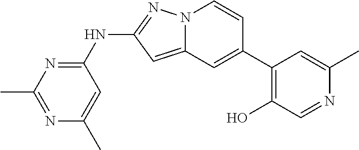

The title compound (6.6 g, 36%) was prepared as described in Intermediate 15 where 4-(2-aminopyrazolo[1,5-a]pyridin-5-yl)-6-methylpyridin-3-ol was used instead of Intermediate 8. ¹H NMR (400 MHz, DMSO-d₆) δ 10.18 (s, 1H), 8.55 (d, J=7.2 Hz, 1H), 8.14 (s, 1H), 7.96 (s, 1H), 7.28 (s, 1H), 7.16-7.11 (m, 1H), 7.02 (d, J=8.1 Hz, 1H), 6.85 (br. s., 1H), 2.46 (s, 3H), 2.40 (s, 3H), 2.30 (s, 3H). MS (ESI): mass calcd. for C₁₉H₁₈N₆O, 346.4. m/z found, 347.1 [M+H]⁺

### Intermediate 17: 645-(5-fluoro-2-methylpyridin-4-yl)pyrazolo[1,5-a]pyridin-2-yl)amino)-2,4-dimethylpyridazin-3(2H)-one

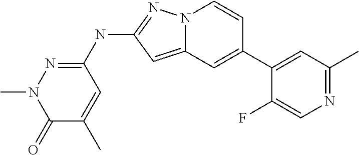

A 30 mL microwave vial containing a stir bar was charged with Intermediate 8 (1.00 g, 4.12 mmol), 6-chloro-2,4-dimethylpyridazin-3(2H)-one (720 mg, 4.54 mmol), tBuBrettPhos Pd G3 (176 mg, 0.21 mmol), cesium carbonate (2.69 g, 8.26 mmol), and 1,4-dioxane (15 mL). The reaction mixture was degassed under vacuum with sonication for 2 minutes, then vented to argon. The vial was placed into a pre-heated plate at 80° C. for 16 h. The reaction mixture was then filtered, concentrated to dryness under reduced pressure to afford the crude product which was purified by flash column chromatography (eluent: 0-10% 2M NH₃ in CH₃OH/DCM)) to afford the title compound (1.20 g, 80%) as a white solid. MS (ESI): mass calcd. for C₁₉H₁₇FN₆O, 364.1. m/z found, 365.2 [M+H]⁺. ¹H NMR (500 MHz, Chloroform-d) δ 8.42-8.35 (m, 2H), 7.75 (t, J=2.1 Hz, 1H), 7.53-7.48 (m, OH), 7.43 (d, J=4.6 Hz, 1H), 7.20 (dt, J=3.0, 1.5 Hz, 1H), 7.00-6.91 (m, 2H), 3.82-3.78 (m, 3H), 3.36 (h, J=1.8 Hz, 1H), 2.62 (d, J=2.9 Hz, 3H), 2.26-2.22 (m, 3H).

### Intermediate 18: (R)-4-(2-aminopyrazolo[1,5-a]pyridin-5-yl)-5-((1-methylpyrrolidin-3-yl)methoxy)picolinonitrile

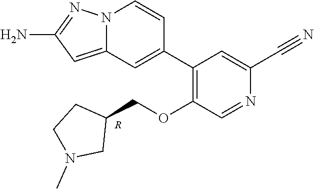

Step A: 5-(2-chloro-5-fluoropyridin-4-yl)pyrazolo[1,5-a]pyridin-2-amine. To a round bottom flask containing 5-bromopyrazolo[1,5-a]pyridin-2-amine (Intermediate 1, 4.29 g, 20.2 mmol), (2-chloro-5-fluoropyridin-4-yl)boronic acid (3.9 g, 22.2 mmol), PdCl₂(dppf) (0.659 g, 1.01 mmol), 1,4-dioxane (80 mL), water (20 mL) and CsF (9.21 g, 60.7 mmol) was evacuated and backfilled with nitrogen (3×). The flask was then placed in a pre-heated heating block at 60° C. After 3 the mixture was extracted (2×EtOAc), the combined organic layers were dried (MgSO₄) and concentrated. The residue was purified (FCC, SiO₂) to provide the title compound (3.2 g, 60%) as a yellow solid. MS (ESI): mass calcd. for C₁₂H₈ClFN₄, 262.04. m/z found, 262.9 [M+H]⁺.

Step B: 4-(2-aminopyrazolo[1,5-a]pyridin-5-yl)-5-fluoropicolinonitrile. To a mixture consisting of 5-(2-chloro-5-fluoropyridin-4-yl)pyrazolo[1,5-a]pyridin-2-amine (2600 mg, 9.90 mmol) and zinc cyanide (1744 mg, 14.85 mmol). DMF (50 mL) was added followed by Pd(PPh₃)₄ (915 mg, 0.792 mmol). The reaction mixture was heated at 120° C. for 3 h. The mixture was cooled to room temperature and concentrated to give the title compound as a crude product (6 g, assumed 100% yield, 42% purity). MS (ESI): mass calcd. for C₁₃H₈FN₅, 253.08. m/z found, 253.9 [M+H]⁺.

Step C: (R)-4-(2-aminopyrazolo[1,5-a]pyridin-5-yl)-5-((1-methylpyrrolidin-3-yl)methoxy)picolinonitrile. A mixture of 4-(2-aminopyrazolo[1,5-a]pyridin-5-yl)-5-fluoropicolinonitrile (6 g crude), (R)-(1-methylpyrrolidin-3-yl)methanol (1.71 g, 14.8 mmol), Cs₂CO₃ (8.06 g, 24.7 mmol), and DMF (60 mL) was stirred at 60° C. overnight. The mixture was concentrated to give a crude product, which was purified by FCC (dichloromethane:methanol from 1:0 to 10:1) to afford the title compound (3.6 g, crude) as a yellow solid. MS (ESI): mass calcd. for C₁₉H₂₀N₆O, 348.17. m/z found, 348.9 [M+H]⁺. ¹H NMR (400 MHz, DMSO-d₆) δ 8.62 (s, 1H), 8.34 (d, J=7.2 Hz, 1H), 8.12 (s, 1H), 7.62 (s, 1H), 6.81 (dd, J=1.2, 7.2 Hz, 1H), 5.73 (s, 1H), 5.39 (s, 2H), 2.63-2.53 (m, 2H), 2.47-2.37 (m, 2H), 2.35-2.28 (m, 1H), 2.22 (s, 3H), 1.98-1.83 (m, 1H), 1.62-1.45 (m, 1H).

### Intermediate 19: 5-(5-fluoro-2-(prop-1-yn-1-yl)pyridin-4-yl)-N-(1-methyl-1H-pyrazol-3-yl)pyrazolo[1,5-a]pyridin-2-amine

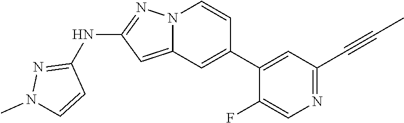 [SMILES: CC#Cc1cc(-c2ccn3nc(Nc4ccn(C)n4)cc3c2)c(F)cn1]

The title compound (520 mg, 55%) was prepared as described in Example 300, Steps B-D, and using 2-chloro-5-fluoro-4-iodopyridine instead of (2R,3S)-2-(((6-chloro-4-iodopyridin-3-yl)oxy)methyl)tetrahydrofuran-3-ol in Step B, and using 3-bromo-1-methyl-1h-pyrazole instead of 4-bromo-1-methyl-1h-pyrazole in Step D. MS (ESI): mass calcd. for C₁₉H₁₅FN₆, 346.4. m/z found, 347.2 [M+H]⁺. ¹H NMR (400 MHz, DMSO-d₆) δ 9.13 (s, 1H), 8.61 (d, J=2.6 Hz, 1H), 8.52 (dd, J=7.1, 1.0 Hz, 1H), 7.85-7.79 (m, 1H), 7.77 (d, J=6.4 Hz, 1H), 7.49 (d, J=2.2 Hz, 1H), 6.92 (dt, J=7.1, 1.9 Hz, 1H), 6.50 (d, J=0.8 Hz, 1H), 6.05 (d, J=2.2 Hz, 1H), 3.73 (s, 3H), 2.09 (s, 3H).

### Intermediate 20: 1-((4-(2-aminopyrazolo[1,5-a]pyridin-5-yl)-6-methylpyridin-3-yl)oxy)-2-methylpropan-2-ol

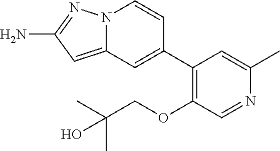 [SMILES: Cc1cc(-c2ccn3nc(N)cc3c2)c(OCC(C)(C)O)cn1]

Step A: 1-((4-bromo-6-methylpyridin-3-yl)oxy)-2-methylpropan-2-ol. To a mixture of 4-bromo-6-methylpyridin-3-ol (1.18 g, 6.28 mmol) and cesium carbonate (6.13 g, 18.8 mmol) in DMF (12.5 mL, 0.5 M) was added isobutylene oxide (1.23 mL, 13.8 mmol). The reaction was stirred at 60° C. for 46 hr, then worked-up in ethyl acetate, water, and brine. The organics were separated, dried with sodium sulfate, then purified via FCC (20-100% ethyl acetate in hexanes) to yield the title compound (702 mg, 43%) as a yellow solid.

Step B: tert-butyl (5-(5-(2-hydroxy-2-methylpropoxy)-2-methylpyridin-4-yl)pyrazolo[1,5-a]pyridin-2-yl)carbamate. To a capped vial with a mixture of 1-((4-bromo-6-methylpyridin-3-yl)oxy)-2-methylpropan-2-ol (700 mg, 2.69 mmol), tert-butyl N-[5-(4,4,5,5-tetramethyl-1,3,2-dioxaborolan-2-yl)pyrazolo[1,5-a]pyridin-2-yl]carbamate (1.21 g, 3.36 mmol), and 1,1′-bis(diphenylphosphino)ferrocene-palladium(ii)dichloride dichloromethane complex (222 mg, 0.27 mmol) was added 1,4-dioxane (10.7 mL) and 0.5 M K₃PO₄ (5.38 mL) under vacuum. The vial was subjected to a nitrogen line, then heated at 70° C. for 20 hr. The reaction mixture was worked-up in ethyl acetate, water, and brine. The organics were separated, dried with sodium sulfate, then purified via FCC (20-100% ethyl acetate in hexanes) to yield the title compound (950 mg, 86%) as a pink solid.

Step C: 1-((4-(2-aminopyrazolo[1,5-a]pyridin-5-yl)-6-methylpyridin-3-yl)oxy)-2-methylpropan-2-ol. To a solution of tert-butyl (5-(5-(2-hydroxy-2-methylpropoxy)-2-methylpyridin-4-yl)pyrazolo[1,5-a]pyridin-2-yl)carbamate (950 mg, 2.3 mmol) in DCE (9.2 mL) was added TFA (1.76 mL, 23 mmol). The mixture was allowed to stir for 6 h, then worked-up in ethyl acetate, water, and brine. The organics were separated, dried with sodium sulfate, then purified via FCC (0-100% ethyl acetate in hexanes) to yield the title compound (530 mg, 74%) as a yellow solid. MS (ESI): mass calcd. for C₁₇H₂₀N₄O₂, 312.4. m/z found, 331.2 [M+H]⁺. ¹H NMR (400 MHz, DMSO-d₆) δ 8.30 (d, J=7.2 Hz, 1H), 8.28 (s, 1H), 7.59 (s, 1H), 7.31 (s, 1H), 6.84-6.78 (m, J=7.0 Hz, 1H), 5.68 (s, 1H), 5.34 (s, 2H), 4.64 (s, 1H), 3.85 (s, 2H), 2.45 (s, 3H), 1.15 (s, 6H)

### Intermediate 21: N-Cyclopropyl-4-((5-(5-fluoro-2-methylpyridin-4-yl)pyrazolo[1,5-a]pyridin-2-yl)amino)-2,6-dimethoxybenzamide

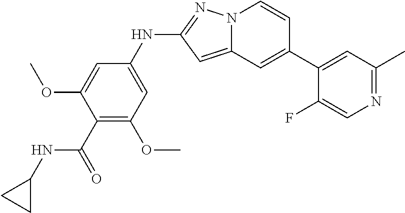 [SMILES: COc1cc(Nc2cc3cc(-c4cc(C)ncc4F)ccn3n2)cc(OC)c1C(=O)NC1CC1]

A 30 mL microwave vial containing a stir bar was charged with Intermediate 8 (500 mg, 2.06 mmol), 4-bromo-N-cyclopropyl-2,6-dimethoxybenzamide (619 mg, 2.06 mmol), tBuBrettPhos Pd G3 (108 mg, 0.13 mmol), cesium carbonate (1.23 g, 3.78 mmol), and 1,4-dioxane (15 mL). The reaction mixture was degassed under vacuum with sonication for 2 minutes, then vented to argon. The vial was placed into a pre-heated plate at 80° C. for 16 h. The reaction mixture was then filtered, concentrated to dryness under reduced pressure to afford the crude product which was purified by flash column chromatography (eluent: 0-10% 2M NH₃ in CH₃OH/DCM)) to afford the title compound (780 mg, 82%) as a white solid. MS (ESI): mass calcd. for C₂₅H₂₄FN₅O₃, 461.2. m/z found, 462.2 [M+H]⁺. ¹H NMR (500 MHz, Chloroform-d) δ 8.41 (t, J=2.2 Hz, 1H), 8.32 (dd, J=10.5, 6.9 Hz, 1H), 7.62-7.56 (m, 1H), 7.30 (d, J=6.6 Hz, 1H), 6.85-6.79 (m, 1H), 6.66 (d, J=1.5 Hz, OH), 6.60 (d, J=1.5 Hz, 2H), 6.24-6.15 (m, 1H), 4.12 (q, J=7.1 Hz, 1H), 3.84-3.74 (m, 7H), 2.93-2.86 (m, 1H), 2.61-2.55 (m, 3H), 0.85-0.78 (m, 2H), 0.63-0.54 (m, 2H).

### Intermediate 22: N-(2,6-dimethylpyrimidin-4-yl)-5-(4,4,5,5-tetramethyl-1,3,2-dioxaborolan-2-yl)pyrazolo[1,5-a]pyridin-2-amine

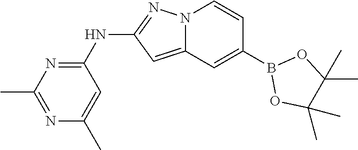

Into a 2-L 4-necked round-bottom flask purged and maintained with an inert atmosphere of nitrogen, was placed Intermediate 14 (65.00 g, 250.00 mmol, 1.00 equiv), 4-chloro-2,6-dimethylpyrimidine (67.70 g, 480.00 mmol, 1.90 equiv), t-BuBrettPhos Pd G3 (10.50 g, 12.50 mmol, 0.05 equiv), Cs₂CO₃ (162.50 g, 500.00 mmol, 2.00 equiv), dioxane (975.00 mL). The resulting solution was stirred for 5 h at 90° C. The reaction mixture was cooled to 25° C. with a water/ice bath. The solids were filtered out. The filtrate was then quenched by the addition of 2 L of water/ice. The resulting solution was extracted with 3×1 L of ethyl acetate and the organic layer was concentrated. The residue was applied onto a silica gel column with ethyl acetate/petroleum ether (1:10). This resulted in 50.3 g (54.9%) of the title compound as a white solid. MS (ESI): mass calcd. for C₁₉H₂₄BN₅O₂, 365.20. m/z found, 366.2 [M+H]⁺. ¹H NMR (300 MHz, DMSO-d₆, ppm) δ 10.21 (s, 1H), 8.50 (d, J=6.9 Hz, 1H), 7.88 (s, 1H), 7.01 (s, 1H), 6.89-6.86 (m, 2H), 2.46 (s, 3H), 2.30 (s, 3H), 1.32 (s, 12H).

### Intermediate 23: methyl 4-((5-(5-(((1r,4r)-4-hydroxycyclohexyl)oxy)-2-methylpyridin-4-yl)pyrazolo[1,5-a]pyridin-2-yl)amino)-2,6-dimethoxybenzoate

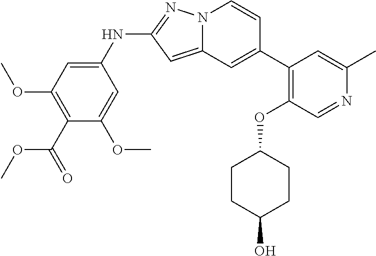

The title compound was prepared by the similar method of Intermediate 15 using methyl 4-bromo-2,6-dimethoxybenzoate instead of 4-chloro-2,6-dimethylpyrimidine (499 mg, 63%). MS (ESI): mass calcd. for C₂₉H₃₂N₄O₆, 532.6. m/z found, 533.2 [M+H]⁺.

### Intermediate 24: 5-(5-(((1R,5S,7s)-3-oxa-9-azabicyclo[3.3.1]nonan-7-yl)oxy)-2-methylpyridin-4-yl)-N-(2,6-dimethylpyrimidin-4-yl)pyrazolo[1,5-a]pyridin-2-amine

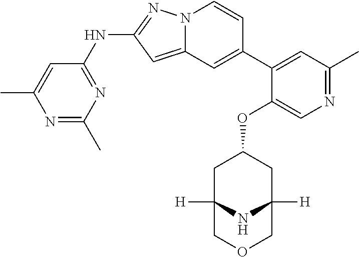

To a solution of (1R,5S,7s)-3-oxa-9-azabicyclo[3.3.1]nonan-7-ol (567 mg, 3.16 mmol) and DMA (1.33 mL) was added NaH (60% dispersion in mineral oil) (229 mg, 5.7 mmol). The mixture was stirred at r.t. for 10 min. Then, N-(2,6-dimethylpyrimidin-4-yl)-5-(5-fluoro-2-methylpyridin-4-yl)pyrazolo[1,5-a]pyridin-2-amine (Intermediate 15) was added. The reaction mixture was stirred at 80° C. for 60 min. The reaction was quenched with H₂O and then extracted with DCM. The organics were collected, dried over anh. MgSO₄, filtered, and concentrated under vacuo. The crude was purified via FCC using 10% MeOH/DCM to afford the title compound (560 mg, 82.7 mmol). MS (ESI): mass calcd. for C₂₆H₂₉N₇O₂, 471.6. m/z found, 472.2 [M+H]⁺. ¹H NMR (500 MHz, Methanol-d₄) δ 8.30 (d, J=7.2 Hz, 1H), 8.06 (s, 1H), 7.96 (d, J=1.8 Hz, 1H), 7.28 (s, 1H), 7.07 (dd, J=7.2, 2.0 Hz, 1H), 6.92 (s, 1H), 6.71 (s, 1H), 4.58-4.50 (m, 1H), 3.75-3.56 (m, 4H), 2.94-2.86 (m, 2H), 2.43-2.41 (two s, 6H), 2.34-2.24 (m, 5H), 1.92-1.82 (m, 2H).

### Intermediate 25: (2R,3S)-2-(((4-(2-aminopyrazolo[1,5-a]pyridin-5-yl)-6-methylpyridin-3-yl)oxy)methyl)tetrahydrofuran-3-ol

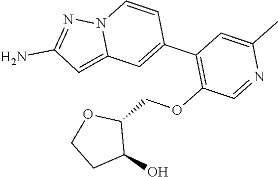 [SMILES: Cc1cc(-c2ccn3nc(N)cc3c2)c(OC[C@H]2OCC[C@@H]2O)cn1]

Step A: ((2R,3S)-3-hydroxytetrahydrofuran-2-yl)methyl 4-methylbenzenesulfonate. To a 1 L round bottom flask was added 1,2-dideoxy-d-ribofuranose (4.62 g, 39.1 mmol), dichloromethane (391 mL, 39.1 mmol), and 4-dimethylaminopyridine (7.17 g, 58.7 mmol). Under ice bath, p-toluenesulfonyl chloride (7.46 g, 39.1 mmol) was slowly added. The reaction mixture was slowly warmed up to room temperature and stirred for 2 days. The mixture was concentrated onto silica, then purified via FCC (0-100% ethyl acetate/hexanes). The second peak to elute was collected as the title compound (4.89 g, 46%).

Step B: (2R,3S)-2-(((4-bromo-6-methylpyridin-3-yl)oxy)methyl)tetrahydrofuran-3-ol. To a round bottom flask was added 4-bromo-6-methylpyridin-3-ol (3.27 g, 17.4 mmol), ((2R,3S)-3-hydroxytetrahydrofuran-2-yl)methyl 4-methylbenzenesulfonate (3.95 g, 14.5 mmol), cesium carbonate (11.3 g, 34.8 mmol), and DMF (41.4 mL, 14.5 mmol). The reaction mixture was heated at 50° C. for 21 h, then at 60° C. for 2 hr. The reaction was worked-up in a separatory funnel using ethyl acetate/water/brine. The product was further extracted from the aqueous layer using 5% methanol in ethyl acetate. The combined organic extracts were dried with Na₂SO₄ and purified via FCC (50-100% ethyl acetate in hexanes) to give the title compound (3.1 g, 74%).

Step C: (2R,3S)-2-(((4-(2-aminopyrazolo[1,5-a]pyridin-5-yl)-6-methylpyridin-3-yl)oxy)methyl)tetrahydrofuran-3-ol. To a round bottom flask was added (2R,3S)-2-4(4-bromo-6-methylpyridin-3-yl)oxy)methyl)tetrahydrofuran-3-ol (2 g, 6.94 mmol), 5-(4,4,5,5-tetramethyl-1,3,2-dioxaborolan-2-yl)pyrazolo[1,5-a]pyridin-2-amine (Intermediate 14, 2.34 g, 9.02 mmol), and 1,1′-bis(diphenylphosphino)ferrocene-palladium(ii)dichloride dichloromethane complex (573 mg, 0.69 mmol). Under vacuum, added 1,4-dioxane (28 mL, 6.94 mmol) and 0.5 M potassium phosphate (13.9 mL, 6.94 mmol). Evacuated and backfilled with nitrogen (3×), then heated mixture at 90° C. under nitrogen for 16 h. Worked-up mixture in separatory funnel using ethyl acetate/water/brine. Dried organic layer with Na₂SO₄, then purified via FCC (initially used 50-100% ethyl acetate in hexanes, but moved to 10% methanol in ethyl acetate when product was not eluting) to give 1.61 g (68%) of the title compound. MS (ESI): mass calcd. for C₁₈H₂₀N₄O₃, 340.2. m/z found, 341.1 [M+H]⁺. ¹H NMR (500 MHz, DMSO-d6) δ 8.32-8.27 (m, 2H), 7.56-7.51 (m, 1H), 7.30 (s, 1H), 6.76 (dd, J=7.2, 2.0 Hz, 1H), 5.70-5.66 (m, 1H), 5.32 (s, 2H), 5.02 (d, J=4.2 Hz, 1H), 4.19-4.06 (m, 3H), 3.88-3.74 (m, 3H), 2.44 (s, 3H), 1.98-1.88 (m, 1H), 1.73-1.64 (m, 1H).

### Intermediate 26: 6-chloro-4-(2-((2,6-dimethylpyrimidin-4-yl)amino)pyrazolo[1,5-a]pyridin-5-yl)pyridin-3-ol

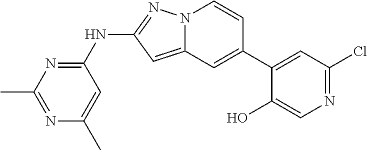 [SMILES: Cc1cc(Nc2cc3cc(-c4cc(Cl)ncc4O)ccn3n2)nc(C)n1]

To a round bottom flask was added 6-chloro-4-iodopyridin-3-ol (2.10 g, 8.21 mmol), N-(2,6-dimethylpyrimidin-4-yl)-5-(4,4,5,5-tetramethyl-1,3,2-dioxaborolan-2-yl)pyrazolo[1,5-a]pyridin-2-amine (Intermediate 22, 3 g, 8.21 mmol), 1,1′-bis(diphenylphosphino)ferrocene-palladium(ii)dichloride dichloromethane complex (678 mg, 0.82 mmol), 1,4-dioxane (32.9 mL, 8.21 mmol), and 0.5 M potassium phosphate (16.4 mL, 8.21 mmol). The flask was evacuated and backfilled with N₂ (3×), then stirred at 70° C. under N₂ for 20 hr. Poured reaction mixture onto 1 L of stirring water and filtered off precipitate (tan color). Transferred precipitate to a separate round bottom flask, then added 200 mL 70% THF/30% ether. Stirred mixture at 50° C. for 30 min. Filtered off solids (grey color), then rinsed with THF/ether. Placed in hi-vac under 70° C. for 30 min to yield 2.34 g (78%) of the title compound. MS (ESI): mass calcd. for C₁₈H₁₅ClN₆O, 366.8. m/z found, 367.1 [M+H]⁺. ¹H NMR (400 MHz, DMSO-d₆) d 10.70 (s, 1H), 10.20 (s, 1H), 8.59 (dt, J=7.3, 1.0 Hz, 1H), 8.10 (s, 1H), 7.95 (dd, J=2.0, 0.9 Hz, 1H), 7.56 (s, 1H), 7.11 (dd, J=7.2, 2.0 Hz, 1H), 7.00 (s, 1H), 6.91 (s, 1H), 2.47 (s, 3H), 2.31 (s, 3H).

### Intermediate 27: 3-((4-(2-aminopyrazolo[1,5-a]pyridin-5-yl)-6-(difluoromethoxy)pyridin-3-yl)oxy)-2,2-dimethylpropanenitrile

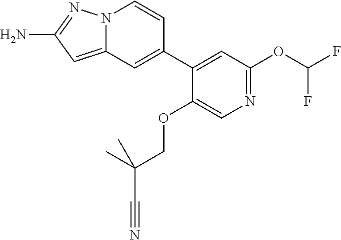 [SMILES: CC(C)(C#N)COc1cnc(OC(F)F)cc1-c1ccn2nc(N)cc2c1]

To a solution of 3-((6-(difluoromethoxy)-4-iodopyridin-3-yl)oxy)-2,2-dimethylpropanenitrile (Example 329 Steps A-C, 10.0 g, 20.4 mmol, 1.00 equiv), Intermediate 15 (9.23 g, 35.7 mmol, 1.75 equiv), Pd(dppf)Cl₂ (1.68 g, 2.04 mmol, 10 mol %), aq. K₃PO₄ (0.5 M, 122 mL, 3.00 equiv) and 1,4-dioxane (82 mL, 0.25 M) was purged with N₂ (evacuated and back-filled with N₂×3). The resulting mixture was heated at 60° C. for 16 hours. The mixture was then cooled to 23° C. and diluted with water (100 mL) and extracted with 10% MeOH in DCM (3×100 mL). The combined organics were dried with MgSO₄, filtered and concentrated in vacuo. The crude oil was purified by purified by silica gel chromatography (10-70% EtOAc in hexanes) to give the title compound (5.78 g, 15.5 mmol, 76% yield) as a tan solid. MS (ESI): mass calcd. for C₁₈H₁₇F₂N₅O₂, 373.1. m/z found, 374.2 [M+H]⁺. ¹H NMR (500 MHz, DMSO-d₆) δ 8.32 (d, J=7.1 Hz, 1H), 8.10 (s, 1H), 7.81-7.48 (m, 2H), 7.21 (s, 1H), 6.80 (dd, J=7.2, 2.0 Hz, 1H), 5.70 (d, J=0.8 Hz, 1H), 5.37 (s, 2H), 4.18 (s, 2H), 1.35 (s, 6H).

### Intermediate 28: 3-((4-(2-amino-4,5-dihydropyrazolo[1,5-a]pyridin-5-yl)-6-(2,2-difluoroethoxy)pyridin-3-yl)oxy)-2,2-dimethylpropanenitrile

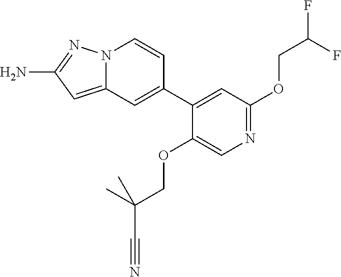 [SMILES: CC(C)(C#N)COc1cnc(OCC(F)F)cc1-c1ccn2nc(N)cc2c1]

Step A: 3-((6-(2,2-difluoroethoxy)-4-iodopyridin-3-yl)oxy)-2,2-dimethylpropanenitrile. —To a flask was charged 3-((6-hydroxy-4-iodopyridin-3-yl)oxy)-2,2-dimethylpropanenitrile (Example 329 Steps A-B, 2.20 g, 6.92 mmol) Cs₂CO₃ (4.50 g, 13.8 mmol) and DMF (32 mL) resulting in a white mixture. Then, to the mixture was added 2,2-difluoroethyl 4-methylbenzenesulfonate (2.45 g, 10.4 mmol) as liquid. The reaction was stirred at 90° C. for 10 h. The mixture was cooled to room temperature and diluted with EtOAc and washed with water (3×). The combined organics were dried with MgSO₄, filtered and concentrated in vacuo. The crude oil was purified by silica gel chromatography (10-100% EtOAc) to give the title compound (2.47 g, 6.46 mmol, 93% yield).

Step B: 3-((4-(2-amino-4,5-dihydropyrazolo[1,5-a]pyridin-5-yl)-6-(2,2-difluoroethoxy)pyridin-3-yl)oxy)-2,2-dimethylpropanenitrile. 3-((4-(2-aminopyrazolo[1,5-a]pyridin-5-yl)-6-(difluoromethoxy)pyridin-3-yl)oxy)-2,2-dimethylpropanenitrile. To a solution of 3-((6-(2,2-difluoroethoxy)-4-iodopyridin-3-yl)oxy)-2,2-dimethylpropanenitrile (2.3 g, 6.02 mmol), Intermediate 14 (2.73 g, 10.5 mmol), Pd(dppf)Cl₂ (0.48 g, 0.602 mmol, 10 mol %), aq. K₃PO₄ (0.5 M, 20 mL, 18.1 mmol) and 1,4-dioxane (24 mL) was purged with N₂ (evacuated and back-filled with N₂×3). The resulting mixture was heated at 70° C. for 3 hours. The mixture was then cooled to 23° C. and diluted with water (100 mL) and extracted with EtOAc (3×100 mL). The combined organics were dried with MgSO₄, filtered and concentrated in vacuo. The crude oil was purified by silica gel chromatography (30-100% EtOAc in hexanes) to give the title compound (1.50 g, 3.87 mmol, 64% yield) as a tan solid. MS (ESI): mass calcd. for C₁₉H₁₉F₂N₅O₂, 387.1. m/z found, 388.1 [M+H]⁺. ¹H NMR (600 MHz, CDCl₃) δ 8.24 (dd, J=7.1, 1.1 Hz, 1H), 7.83 (s, 1H), 7.56 (dd, J=2.2, 1.0 Hz, 1H), 6.91 (s, 1H), 6.85-6.76 (m, 1H), 6.16 (tt, J=55.7, 4.2 Hz, 1H), 5.88 (s, 1H), 4.55 (td, J=13.6, 4.2 Hz, 2H), 4.04 (s, 2H), 3.91 (d, J=1.1 Hz, 2H), 1.41 (d, J=1.2 Hz, 6H).

### Example 1: N-[5-[2-Cyano-5-[[(3R)-1-methylpyrrolidin-3-yl]methoxy]-4-pyridyl]pyrazolo[1,5-a]pyridin-2-yl]cyclopropanecarboxamide

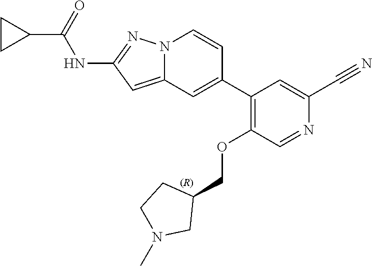

A 50 mL round-bottom flask was charged with sodium hydride (NaH) (60% dispersion in mineral oil, 114 mg, 2.85 mmol) as a solid, followed by addition of 6 mL of DMF (12 mL, 0.1 M, 1.2 mmol). The round-bottom flask was placed into an ice-bath and cooled to about 0° C. A solution of N-(5-(5-chloro-2-cyanopyridin-4-yl)pyrazolo[1,5-a]pyridin-2-yl)cyclopropanecarboxamide (Intermediate 5, 424 mg, 1.243 mmol) in 3 mL DMF was then added to the mixture dropwise. The mixture turned a yellow/brown color. The resulting mixture was stirred for 5 min then a solution for (R)-(1-methylpyrrolidin-3-yl)methanol (213 mg, 1.85 mmol) in 3 mL DMF was added dropwise. The mixture was stirred for 5 min then allowed to warm to rt. The reaction mixture was stirred for additional 70 minutes, at which time it was poured into 100 mL of 5% aq. LiCl. The mixture was extracted 3×100 mL EtOAc. The combined organic layers were concentrated in-vacuo in the presence of 5 g of diatomaceous earth. The product was purified via FCC using 0-70% 2M 10% NH₃/MeOH-DCM gradient to afford the title compound (353 mg, 66%). MS (ESI): mass calcd. for C₂₃H₂₄N₆O₂, 416.2. m/z found, 417.2 [M+H]⁺. ¹H NMR (400 MHz, DMSO-d₆) δ 11.11 (s, 1H), 8.65 (s, 1H), 8.61 (d, J=7.3 Hz, 1H), 8.18 (s, 1H), 7.93 (d, J=1.9 Hz, 1H), 7.10 (dd, J=7.3, 2.0 Hz, 1H), 6.91 (s, 1H), 4.21 (d, J=6.6 Hz, 2H), 2.62-2.52 (m, 2H), 2.46-2.28 (m, 3H), 2.20 (s, 3H), 1.91 (dddd, J=17.6, 13.1, 8.1, 5.6 Hz, 2H), 1.60-1.45 (m, 1H), 0.82 (tq, J=8.2, 5.4, 4.2 Hz, 4H).

### Example 2: N-[5-[2-cyano-5-(2-dimethylaminoethylamino)-4-pyridyl]pyrazolo[1,5-a]pyridin-2-yl]cyclopropanecarboxamide

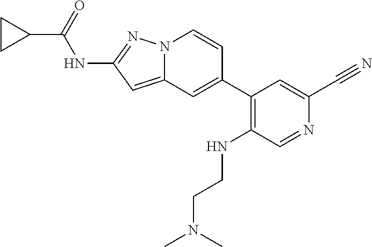 [SMILES: CN(C)CCNc1cnc(C#N)cc1-c1ccn2nc(NC(=O)C3CC3)cc2c1]

To a 10 mL vial was charged with N-(5-(5-chloro-2-cyanopyridin-4-yl)pyrazolo[1,5-a]pyridin-2-yl)cyclopropanecarboxamide (Intermediate 5, 146 mg, 0.428 mmol), N,N-dimethylethylenediamine (0.081 mL, 0.705 mmol), Cs₂CO₃ (302 mg, 0.927 mmol), chloro(2-dicyclohexylphosphino-2′,6′-diisopropoxy-1,1′biphenyl)[2-(2′-amino-1,1′-biphenyl)]palladium(II) (9.2 mg, 0.0118 mmol), and dicyclohexyl(2′,6′-diisopropoxy-[1,1′-biphenyl]-2-yl)phosphine (5.4 mg, 0.0116 mmol) as solids. The vial was evacuated and backfilled 2× with argon. To the vial was added 1,4-dioxane (1 mL, 0.4 M, 0.4 mmol) and it was placed on a preheated 100° C. heating block for 70 min. Then, the reaction mixture was allowed to cool to room temperature. The reaction mixture was diluted with 50 mL of DCM and 1 g of diatomaceous earth was added, and then the solvent was evaporated under reduced. The product was purified via FCC using 0-10% 2M NH₃-MeOH-DCM/DCM gradient to afford a white powder (129 mg, 69%). MS (ESI): mass calcd. for C₂₁H₂₃N₇O, 389.2. m/z found, 390.2 [M+H]⁺. ¹H NMR (400 MHz, CDCl₃) δ 8.55 (s, 1H), 8.31 (dd, J=7.1, 1.0 Hz, 1H), 8.12 (s, 1H), 7.51-7.45 (m, 1H), 7.40 (s, 1H), 7.00 (s, 1H), 6.68 (dd, J=7.2, 2.0 Hz, 1H), 5.50 (t, J=4.6 Hz, 1H), 3.36-3.21 (m, 2H), 2.56 (dd, J=6.6, 5.1 Hz, 2H), 2.18 (s, 6H), 1.59-1.50 (m, 1H), 1.18-1.07 (m, 2H), 0.95-0.85 (m, 2H).

### Example 3: N-[5-[2-cyano-5-[3-(hydroxymethyl)azetidin-1-yl]-4-pyridyl]pyrazolo[1,5-a]pyridin-2-yl]cyclopropanecarboxamide

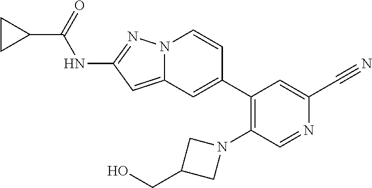 [SMILES: N#Cc1cc(-c2ccn3nc(NC(=O)C4CC4)cc3c2)c(N2CC(CO)C2)cn1]

Pd₂(dba)₃ (51 mg, 0.056 mmol) was added to a solution of N-(5-(5-chloro-2-cyanopyridin-4-yl)pyrazolo[1,5-a]pyridin-2-yl)cyclopropanecarboxamide (150 mg, 0.444 mmol), azetidin-3-ylmethanol (77 mg, 0.88 mmol), Cs₂CO₃ (257 mg, 0.789 mmol), BINAP (51 mg, 0.082 mmol) and 1,4-dioxane (2 mL) under Ar. The reaction mixture was heated at 90° C. for 16 h. The reaction mixture was partitioned between H₂O (15 mL) and ethyl acetate (15 mL), the aqueous phase was extracted with ethyl acetate (15 mL×3) and the combined extracts were dried over anhydrous Na₂SO₄, filtered and concentrated to dryness under reduced pressure to afford the product which was purified via FCC using 0-50% of petroleum ether:ethyl acetate gradient to afford the title compound as a white solid (37 mg, 21%). MS (ESI): mass calcd. for C₂₁H₂₀N₆O₂, 388.2. m/z found, 389.2 [M+H]⁺. ¹H NMR (400 MHz, DMSO-d₆) δ 11.12 (s, 1H), 8.58 (d, J=7.2 Hz, 1H), 8.00 (s, 1H), 7.71 (s, 1H), 7.58 (d, J=1.2 Hz, 1H), 6.89 (s, 1H), 6.83 (dd, J=2.0, 7.2 Hz, 1H), 4.70 (t, J=5.2 Hz, 1H), 3.86-3.76 (m, 2H), 3.57-3.48 (m, 2H), 3.47-3.40 (m, 2H), 2.73-2.61 (m, 1H), 1.97-1.88 (m, 1H), 0.88-0.76 (m, 4H).

### Example 4: (R)-5-((1-methylpyrrolidin-3-yl)methoxy)-4-(2-(pyridin-3-ylamino)pyrazolo[1,5-a]pyridin-5-yl)picolinonitrile

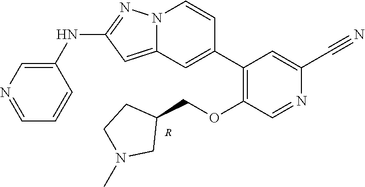

A mixture of (R)-4-(2-aminopyrazolo[1,5-a]pyridin-5-yl)-5-((1-methylpyrrolidin-3-yl)methoxy)picolinonitrile (200 mg, 0.574 mmol), 3-bromopyridine (99.8 mg, 0.631 mmol), Cs₂CO₃ (374 mg, 1.15 mmol), Pd₂(dba)₃ (26.3 mg, 0.029 mmol), xantphos (16.6 mg, 0.029) and 1,4-dioxane (15 mL) was heated overnight at 100° C. under N₂ atmosphere. The reaction mixture was concentrated under reduced pressure to give the product, which was purified by preparative HPLC with Boston Prime C18 150×30 mm×5 um, column (eluent: 35% to 65% (v/v) CH₃CN and H₂O with 0.04% aq. NH₄OH+10 mM NH₄HCO₃) to afford yellow solid (107 mg, 43%). MS (ESI): mass calcd. for C₂₄H₂₃N₇O, 425.2. m/z found, 426.2 [M+H]⁺. ¹H NMR (400 MHz, DMSO-d₆) δ 9.28 (s, 1H), 8.72 (d, J=2.4 Hz, 1H), 8.68-8.61 (m, 2H), 8.18 (s, 1H), 8.09-8.01 (m, 2H), 7.82 (d, J=0.8 Hz, 1H), 7.33-7.26 (m, 1H), 6.99 (dd, J=2.0, 7.2 Hz, 1H), 6.19 (s, 1H), 4.27-4.17 (m, 2H), 2.60-2.54 (m, 2H), 2.45-2.39 (m, 2H), 2.36-2.29 (m, 1H), 2.22 (s, 3H), 1.98-1.84 (m, 1H), 1.60-1.49 (m, 1H).

### Example 5: N-[5-[2-cyano-5-[(3S)-1-(2,2,2-trifluoroethyl)pyrrolidin-3-yl]oxy-4-pyridyl]pyrazolo[1,5-a]pyridin-2-yl]cyclopropanecarboxamide

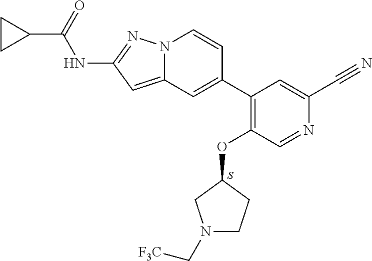

To a solution of (S)—N-(5-(2-cyano-5-(pyrrolidin-3-yloxy)pyridin-4-yl)pyrazolo[1,5-a]pyridin-2-yl)cyclopropanecarboxamide (Example 6, 50 mg, 0.13 mmol), DIPEA (33.3 mg, 0.257 mmol) in DMF (2 mL) was added 2,2,2-trifluoroethyl trifluoromethanesulfonate (44.8 mg, 0.193 mmol). The resulting mixture was stirred at room temperature for 16 h. The mixture was then poured into water (40 mL) and extracted with ethyl acetate (20 mL×3). The organic extracts were washed with brine (20 mL×3), dried over anhydrous Na₂SO₄, filtered, and concentrated to dryness under reduced pressure to obtain the product, which was initially purified by FCC (eluent: methanol:ethylacetate=0:1 to 1:1) and then further purified by SFC over DAICEL CHIRALPAK AD-H 250 mm×30 mm, 5 μm (eluent: 45% to 45% (v/v) 0.1% NH₃—H₂O in IPA) to afford the titled compound as a white solid (8.10 mg, 13%). MS (ESI): mass calcd. for C₂₃H₂₁F₃N₆O₂, 470.2. m/z found, 471.2 [M+H]⁺. ¹H NMR (400 MHz, DMSO-d₆) δ 11.13 (s, 1H), 8.63 (s, 1H), 8.58 (d, J=7.2 Hz, 1H), 8.20 (s, 1H), 7.97-7.94 (m, 1H), 7.13 (dd, J=2.0, 7.6 Hz, 1H), 6.92 (s, 1H), 5.35-5.29 (m, 1H), 3.32-3.25 (m, 2H), 3.17-3.10 (m, 1H), 2.98-2.89 (m, 2H), 2.76-2.67 (m, 1H), 2.40-2.29 (m, 1H), 1.98-1.85 (m, 2H), 0.86-0.80 (m, 4H).

### Example 6: (S)—N-(5-(2-cyano-5-(pyrrolidin-3-yloxy)pyridin-4-yl)pyrazolo[1,5-a]pyridin-2-yl)cyclopropanecarboxamide

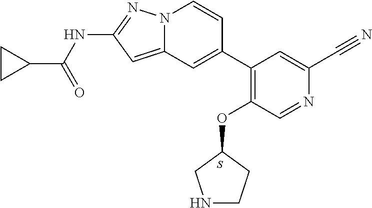

To a 50 mL flask was charged NaH (121 mg, 3.03 mmol, 60% in mineral oil) as a solid, followed by addition of 15 mL DMF. The flask was placed into an ice-bath and cooled to 0° C. A solution of N-(5-(2-cyano-5-fluoropyridin-4-yl)pyrazolo[1,5-a]pyridin-2-yl)cyclopropanecarboxamide (Intermediate 4, 400 mg, 1.01 mmol) in 3 mL DMF was then added to the mixture dropwise. The resulting mixture was stirred for 5 min before dropwise addition of (S)-pyrrolidin-3-ol (175.7 mg, 2.017 mmol) in 2 mL DMF. The mixture was allowed to warm to room temperature for 1 h, then quenched with water (50 mL) and extracted with ethyl acetate (50 mL×3). The organic extracts were washed with brine (50 mL×3), dried over anhydrous Na₂SO₄, filtered, and concentrated to dryness under reduced pressure to obtain the product, which was purified by FCC (eluent: methanol (contain 10% ammonia):ethylacetate=0:1 to 1:1) to afford the title compound as a yellow solid (165.9 mg, 42%). MS (ESI): mass calcd. for C₂₁H₂₀N₆O₂, 388.2. m/z found, 389.0 [M+H]⁺. ¹H NMR (400 MHz, DMSO-d₆) δ 11.14 (s, 1H), 8.65 (s, 1H), 8.59 (d, J=7.6 Hz, 1H), 8.19 (s, 1H), 7.93 (d, J=1.2 Hz, 1H), 7.11 (dd, J=2.0, 7.2 Hz, 1H), 6.93 (s, 1H), 5.30-5.23 (m, 1H), 3.17-3.11 (m, 1H), 3.02-2.95 (m, 1H), 2.94-2.88 (m, 1H), 2.87-2.79 (m, 1H), 2.17-2.05 (m, 1H), 1.98-1.90 (m, 1H), 1.89-1.81 (m, 1H), 0.86-0.79 (m, 4H).

### Example 7: N-[5-[2-cyano-5-[(3S)-1-cyclopropylpyrrolidin-3-yl]oxy-4-pyridyl]pyrazolo[1,5-a]pyridin-2-yl]cyclopropanecarboxamide

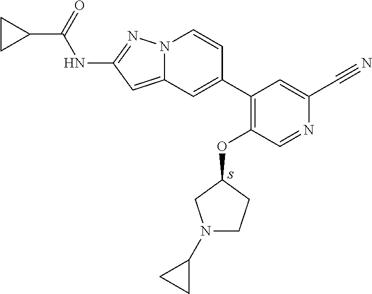

To a solution of (S)—N-(5-(2-cyano-5-(pyrrolidin-3-yloxy)pyridin-4-yl)pyrazolo[1,5-a]pyridin-2-yl)cyclopropanecarboxamide (100 mg, 0.257 mmol), (1-ethoxycyclopropyl)trimethylsilane (89.8 mg, 0.515 mmol) and MeOH (2 mL) was added HOAc (126 mg, 0.515 mmol). The mixture was stirred at room temperature for 10 min then NaBH₃CN (32.4 mg, 0.515 mmol) was added. The resulting mixture was stirred at room temperature for 12 h. The mixture was poured into water (40 mL) and extracted with ethyl acetate (3×20 mL). The organic extracts were washed with brine (3×20 mL), dried over anhydrous Na₂SO₄, filtered, and concentrated to dryness under reduced pressure to obtain the product, which was purified by FCC (eluent: methanol:ethyl acetate=0:1 to 1:1) to afford the product which was further purified by SFC using a REGIS (s,s) WHELK-O1 250 mm×30 mm, 5 μm column (eluent: 50% to 50% (v/v) 0.1% NH₃—H₂O in EtOH) to afford the titled compound (14 mg, 12%). MS (ESI): mass calcd. for C₂₄H₂₄N₆O₂, 428.2. m/z found, 429.2 [M+H]⁺. ¹H NMR (400 MHz, DMSO-d₆) δ 11.14 (s, 1H), 8.64-8.57 (m, 2H), 8.19 (s, 1H), 7.95 (d, J=1.2 Hz, 1H), 7.12 (dd, J=2.0, 7.2 Hz, 1H), 6.92 (s, 1H), 5.29-5.23 (m, 1H), 3.08-3.01 (m, 1H), 2.87-2.78 (m, 2H), 2.64-2.56 (m, 1H), 2.39-2.26 (m, 1H), 1.98-1.89 (m, 1H), 1.87-1.77 (m, 1H), 1.71-1.64 (m, 1H), 0.88-0.79 (m, 4H), 0.42-0.36 (m, 2H), 0.36-0.31 (m, 1H), 0.28-0.23 (m, 1H).

### Example 8: N-(5-(2-cyano-5-(((2*S,4*R)-2-(trifluoromethyl)piperidin-4-yl)oxy)pyridin-4-yl)pyrazolo[1,5-a]pyridin-2-yl)cyclopropanecarboxamide

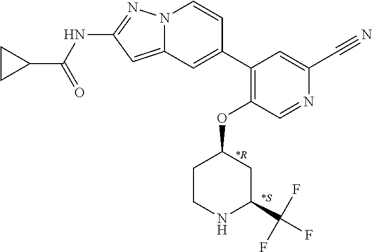

Step A: tert-butyl (2S,4R)-4-((6-cyano-4-(2-(cyclopropanecarboxamido)pyrazolo[1,5-a]pyridin-5-yl)pyridin-3-yl)oxy)-2-(trifluoromethyl)piperidine-1-carboxylate. To a 40 mL flask was charged NaH (408.49 mg, 10.213 mmol, 60% in mineral oil) as a solid followed by addition of 10 mL DMF. The flask was placed into an ice bath and cooled to 0° C. A solution of N-(5-(2-cyano-5-fluoropyridin-4-yl)pyrazolo[1,5-a]pyridin-2-yl)cyclopropanecarboxamide (Intermediate 4, 1100 mg, 3.4 mmol) in 5 mL DMF was then added to the mixture dropwise. The resulting mixture was stirred for 5 min then a solution of (2S,4R)-tert-butyl 4-hydroxy-2-(trifluoromethyl)piperidine-1-carboxylate (1.1 g, 4.1 mmol) in 5 mL DMF was added dropwise. Again, the mixture was stirred for 5 min then allowed to warm to room temperature. The reaction mixture was stirred at room temperature for 1 h, then poured into water (50 mL) and extracted with ethyl acetate (3×50 mL). The organic extracts were washed with brine (3×50 ml), dried over anhydrous Na₂SO₄, filtered, and concentrated in-vacuo to obtain the crude, which was purified by FCC (eluent: petroleum ether:ethylacetate=1:0 to 1:2) to afford the product (770.2 mg, 33%) as white solid which was used as is without further purification.

Step B: N-(5-(2-cyano-5-(42S,4R)-2-(trifluoromethyl)piperidin-4-yl)oxy)pyridin-4-yl)pyrazolo[1,5-a]pyridin-2-yl)cyclopropanecarboxamide. TFA (0.890 g, 7.80 mmol) was added to a solution of (2S,4R)-tert-butyl 4-((6-cyano-4-(2-(cyclopropanecarboxamido)pyrazolo[1,5-a]pyridin-5-yl)pyridin-3-yl)oxy)-2-(trifluoromethyl)piperidine-1-carboxylate (660 mg, 0.976 mmol) and DCM (30 mL). The reaction mixture was quenched with saturated NaHCO₃ (100 mL), then poured into water (50 mL) and extracted with ethyl acetate (3×50 mL). The organic extracts were washed with brine (3×50 mL), dried over anhydrous Na₂SO₄, filtered, and concentrated to dryness under reduced pressure to obtain the crude product, which was purified by FCC (eluent: methanol (contain 10% ammonia): ethylacetate=0:1 to 1:9) to afford the racemic mixture of the titled compound (518 mg, 85%). MS (ESI): mass calcd. for C₂₃H₂₁F₃N₆O₂, 470.2. m/z found, 471.2 [M+H]⁺. ¹H NMR (400 MHz, DMSO-d₆) δ 11.14 (s, 1H), 8.81 (s, 1H), 8.61 (d, J=6.8 Hz, 1H), 8.19 (s, 1H), 7.91 (d, J=0.8 Hz, 1H), 7.09 (dd, J=2.0, 7.6 Hz, 1H), 6.93 (s, 1H), 4.96-4.87 (m, 1H), 3.46-3.38 (m, 2H), 3.08-3.01 (m, 1H), 2.70-2.58 (m, 1H), 2.34-2.25 (m, 1H), 2.12-2.04 (m, 1H), 1.97-1.89 (m, 1H), 1.52-1.30 (m, 2H), 0.87-0.78 (m, 4H). ¹⁹F NMR (376 MHz, DMSO-d₆) δ −75.45 (d, J=7.5 Hz, 3F).

### Example 9: N-(5-(2-cyano-5-((4-(trifluoromethyl)piperidin-4-yl)oxy)pyridin-4-yl)pyrazolo[1,5-a]pyridin-2-yl)cyclopropanecarboxamide

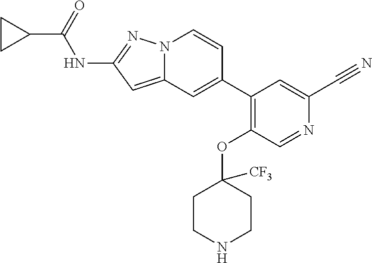 [SMILES: N#Cc1cc(-c2ccn3nc(NC(=O)C4CC4)cc3c2)c(OC2(C(F)(F)F)CCNCC2)cn1]

Step A: tert-butyl 4-((6-cyano-4-(2-(cyclopropanecarboxamido)pyrazolo[1,5-a]pyridin-5-yl)pyridin-3-yl)oxy)-4-(trifluoromethyl)piperidine-1-carboxylate.

NaH (75 mg, 1.9 mmol, 60% in mineral oil) was added to a 0° C. (ice/water) solution of N-(5-(2-cyano-5-fluoropyridin-4-yl)pyrazolo[1,5-a]pyridin-2-yl)cyclopropanecarboxamide (Intermediate 4, 200 mg, 0.622 mmol) and DMF (10 mL). The mixture was stirred for 10 minutes, and then treated with a solution consisting of tert-butyl 4-hydroxy-4-(trifluoromethyl)piperidine-1-carboxylate (218 mg, 0.809 mmol) and DMF (10 mL) was added dropwise. The resultant mixture was stirred at 0° C. for 2 h. The reactant mixture was quenched with water (50 mL) and extracted with ethyl acetate (50 mL×3). The organic extracts were washed with brine (50 mL×3), dried over anhydrous Na₂SO₄, filtered, and concentrated to dryness under reduced pressure to obtain the crude product, which was purified by flash column chromatography (eluent: petroleum ether:ethyl acetate=1:0 to 0:1) to afford the product (200 mg, 44.3%) as yellow oil. MS (ESI): mass calcd. for C₂₈H₂₉F₃N₆O₄, 570.22; m/z 515.2 [M+H-tBu]⁺.

Step B: N-(5-(2-cyano-5-((4-(trifluoromethyl)piperidin-4-yl)oxy)pyridin-4-yl)pyrazolo[1,5-a]pyridin-2-yl)cyclopropanecarboxamide. Trifluoroacetic acid (3 mL) was added to a solution of tert-butyl 4-((6-cyano-4-(2-(cyclopropanecarboxamido)pyrazolo[1,5-a]pyridin-5-yl)pyridin-3-yl)oxy)-4-(trifluoromethyl)piperidine-1-carboxylate (200 mg, 0.276 mmol, 78.6% purity) and methylene chloride (20 mL). The reaction mixture was stirred at room-temperature for 2 hours. The reaction mixture was concentrated to dryness under reduced pressure to afford the crude product which was dissolved in water (10 mL), the aqueous phase was adjusted to pH=8-9 by saturated NaHCO₃ and extracted with ethyl acetate (50 mL×3), the organic layers were dried over anhydrous Na₂SO₄, which was purified by FCC (eluent: methanol (contain 10% ammonia): ethyl acetate=0:1 to 1:1) to afford the title compound (100 mg, 76%) as a white solid. MS(ESI): mass calcd. for C₂₃H₂₁F₃N₆O₂, 470.17; m/z 471.2 [M+H]⁺. ¹H NMR (400 MHz, DMSO-d₆) δ 11.15 (s, 1H), 8.68 (s, 1H), 8.65 (d, J=7.6 Hz, 1H), 8.31 (s, 1H), 7.90 (d, J=1.6 Hz, 1H), 7.05 (dd, J=2.0, 7.2 Hz, 1H), 6.95 (s, 1H), 2.71-2.55 (m, 2H), 2.25-2.12 (m, 2H), 2.04-1.87 (m, 4H), 1.80-1.68 (m, 2H), 0.92-0.74 (m, 4H).

### Example 10: N-[5-[2-cyano-5-[[1-methyl-4-(trifluoromethyl)-4-piperidyl]oxy]-4-pyridyl]pyrazolo[1,5-a]pyridin-2-yl]cyclopropanecarboxamide

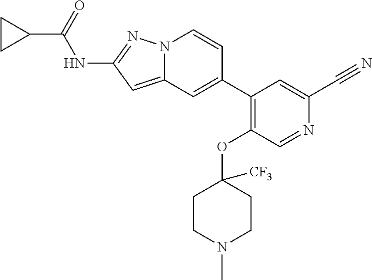 [SMILES: CN1CCC(Oc2cnc(C#N)cc2-c2ccn3nc(NC(=O)C4CC4)cc3c2)(C(F)(F)F)CC1]

H(CHO)n (28.7 mg, 0.319 mmol) was added to a solution consisting of N-(5-(2-cyano-5-((4-(trifluoromethyl)piperidin-4-yl)oxy)pyridin-4-yl)pyrazolo[1,5-a]pyridin-2-yl)cyclopropanecarboxamide (100 mg, 0.213 mmol) and MeOH (15 mL). The reaction mixture was stirred at room-temperature for 30 min, and then NaBH₃CN (40 mg, 0.64 mmol) was added to the reaction mixture. The resultant mixture was stirred at room-temperature for 3 hours. The reactant mixture was quenched with water (50 mL) and extracted with ethyl acetate (50 mL×3). The organic extracts were washed with brine (50 mL×3), dried over anhydrous Na₂SO₄, filtered, and concentrated to dryness under reduced pressure to obtain the crude product, which was purified by flash column chromatography (eluent: methanol (contain 10% aq. ammonia) δ ethyl acetate=0:1 to 1:1) to afford the product. The post chromatographic product was further purified by preparative HPLC with a Boston Prime C18 150×30 mm×5 μm column (eluent: 40% to 70% (v/v) CH₃CN and H₂O with 0.04% NH₃) to afford the title compound (31.3 mg, 30%) as a white solid. MS (ESI): mass calcd. for C₂₄H₂₃F₃N₆O₂, 484.2. m/z found, 485.2 [M+H]⁺. ¹H NMR (400 MHz, DMSO-d₆) δ 11.15 (s, 1H), 8.68 (s, 1H), 8.66 (d, J=7.2 Hz, 1H), 8.30 (s, 1H), 7.89 (s, 1H), 7.02 (dd, J=1.6, 7.2 Hz, 1H), 6.96 (s, 1H), 2.44-2.35 (m, 2H), 2.13-2.01 (m, 2H), 1.98-1.80 (m, 6H), 1.53 (t, J=11.2 Hz, 2H), 0.90-0.78 (m, 4H).

### Example 11: N-[5-[2-cyano-5-[[(4R)-3,3-difluoro-1-methyl-4-piperidyl]oxy]-4-pyridyl]pyrazolo[1,5-a]pyridin-2-yl]cyclopropanecarboxamide

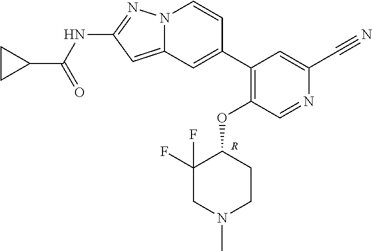

The title compound (17.4 mg, 17%) was prepared as described in Example 9 where (R)-tert-butyl 3,3-difluoro-4-hydroxypiperidine-1-carboxylate was used instead of tert-butyl 4-hydroxy-4-(trifluoromethyl)piperidine-1-carboxylate in Step A, followed by the procedure in Example 10. MS (ESI): mass calcd. for C₂₃H₂₂F₂N₆O₂, 452.2. m/z found, 453.2 [M+H]⁺. ¹H NMR (400 MHz, CD₃OD) δ 8.69 (s, 1H), 8.46 (d, J=7.2 Hz, 1H), 8.01 (s, 1H), 7.81 (d, J=1.2 Hz, 1H), 7.04 (dd, J=2.0, 7.2 Hz, 1H), 6.94 (s, 1H), 5.12-5.03 (m, 1H), 2.87-2.67 (m, 2H), 2.59-2.46 (m, 2H), 2.28 (s, 3H), 2.22-1.99 (m, 2H), 1.92-1.80 (m, 1H), 1.05-0.97 (m, 2H), 0.94-0.84 (m, 2H). ¹⁹F NMR (376 MHz, CD₃OD) δ −108.43-110.63 (m, 1F), −113.69-117.38 (m, 1F).

### Example 12: N-[5-[2-cyano-5-[[(6R)-4-methyl-1,4-oxazepan-6-yl]oxy]-4-pyridyl]pyrazolo[1,5-a]pyridin-2-yl]cyclopropanecarboxamide

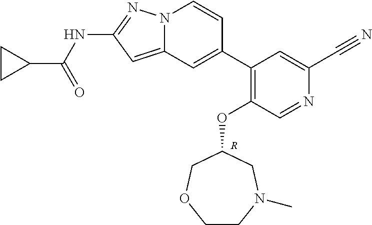

The title compound (41.4 mg, 27%) was prepared as described in Example 9 where (R)-tert-butyl 6-hydroxy-1,4-oxazepane-4-carboxylate was used instead of tert-butyl 4-hydroxy-4-(trifluoromethyl)piperidine-1-carboxylate in Step A, followed by the procedure in Example 10. MS (ESI): mass calcd. for C₂₃H₂₄N₆O₃, 432.2. m/z found, 433.2 [M+H]⁺. ¹H NMR (400 MHz, DMSO-d₆) δ 11.15 (s, 1H), 8.66 (s, 1H), 8.62 (d, J=7.6 Hz, 1H), 8.19 (s, 1H), 8.00 (s, 1H), 7.17 (dd, J=2.0, 7.2 Hz, 1H), 6.92 (s, 1H), 5.18-5.08 (m, 1H), 4.06 (dd, J=4.8, 13.2 Hz, 1H), 3.85 (dd, J=4.0, 13.2 Hz, 1H), 3.75-3.67 (m, 1H), 3.64-3.56 (m, 1H), 3.01 (dd, J=4.8, 13.6 Hz, 1H), 2.77 (dd, J=6.0, 13.2 Hz, 1H), 2.59 (t, J=4.8 Hz, 2H), 2.32 (s, 3H), 1.99-1.89 (m, 1H), 0.88-0.77 (m, 4H).

### Example 13: N-[5-[2-cyano-5-[[(1R,4R,5R)-2-methyl-2-azabicyclo[2.2.1]heptan-5-yl]oxy]-4-pyridyl]pyrazolo[1,5-a]pyridin-2-yl]cyclopropanecarboxamide

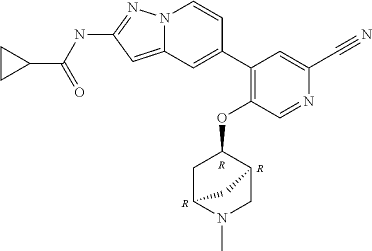

The title compound (11.7 mg, 11%) was prepared as described in Example 9 where (1R,4R,5R)-tert-butyl 5-hydroxy-2-azabicyclo[2.2.1]heptane-2-carboxylate was used instead of tert-butyl 4-hydroxy-4-(trifluoromethyl)piperidine-1-carboxylate in Step A, followed by the procedure in Example 10. MS (ESI): mass calcd. for C₂₄H₂₄N₆O₂, 428.2. m/z found, 429.3 [M+H]⁺. ¹H NMR (400 MHz, DMSO-d₆) δ 11.15 (s, 1H), 8.65 (d, J=7.2 Hz, 1H), 8.58 (s, 1H), 8.18 (s, 1H), 7.98-7.86 (m, 1H), 7.10 (dd, J=1.6, 7.2 Hz, 1H), 6.92 (s, 1H), 5.15-5.04 (m, 1H), 3.15-3.04 (m, 1H), 2.82-2.71 (m, 2H), 2.47-2.43 (m, 2H), 2.23 (s, 3H), 2.15-2.06 (m, 1H), 1.99-1.87 (m, 1H), 1.80-1.71 (m, 1H), 1.57-1.42 (m, 2H), 0.89-0.76 (m, 4H).

### Example 14: N-(5-(2-cyano-5-(((2*R,4*S)-2-(trifluoromethyl)piperidin-4-yl)oxy)pyridin-4-yl)pyrazolo[1,5-a]pyridin-2-yl)cyclopropanecarboxamide

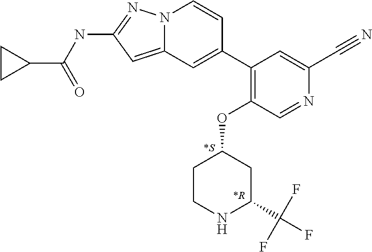

The title compound (21 mg, 13%) was prepared as described in Example 8 using (2R,4S)-2-(trifluoromethyl)piperidin-4-ol in place of (2S,4R)-tert-butyl 4-hydroxy-2-(trifluoromethyl)piperidine-1-carboxylate. MS (ESI): mass calcd. for C₂₃H₂₁F₃N₆O₂, 470.2. m/z found, 471.2 [M+H]⁺. ¹H NMR (400 MHz, DMSO-d₆) δ 11.13 (s, 1H), 8.81 (s, 1H), 8.61 (d, J=6.8 Hz, 1H), 8.19 (s, 1H), 7.91 (d, J=1.2 Hz, 1H), 7.09 (dd, J=2.0, 7.6 Hz, 1H), 6.93 (s, 1H), 4.97-4.87 (m, 1H), 3.51-3.37 (m, 2H), 3.09-3.00 (m, 1H), 2.69-2.59 (m, 1H), 2.33-2.25 (m, 1H), 2.13-2.05 (m, 1H), 1.97-1.89 (m, 1H), 1.51-1.31 (m, 2H), 0.87-0.80 (m, 4H). ¹⁹F NMR (376 MHz, DMSO-d₆) δ −75.45 (d, J=7.1 Hz, 3F).

### Example 15: N-[5-[2-cyano-5-[[(4R)-3,3-difluoro-4-piperidyl]oxy]-4-pyridyl]pyrazolo[1,5-a]pyridin-2-yl]cyclopropanecarboxamide

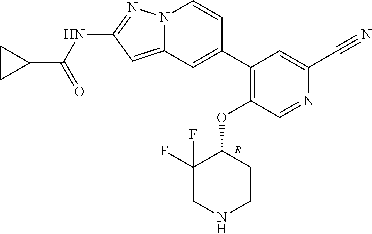

The title compound (10.3 mg, 5.3%) was prepared as described in Example 8 where (R)-tert-butyl 3,3-difluoro-4-hydroxypiperidine-1-carboxylate was used instead of (2S,4R)-tert-butyl 4-hydroxy-2-(trifluoromethyl)piperidine-1-carboxylate. MS (ESI): mass calcd. for C₂₂H₂₀F₂N₆O₂, 438.2. m/z found, 439.2 [M+H]⁺. ¹H NMR (400 MHz, DMSO-d₆) δ 11.15 (s, 1H), 8.83 (s, 1H), 8.65 (d, J=7.2 Hz, 1H), 8.23 (s, 1H), 7.92 (s, 1H), 7.09 (dd, J=1.6, 7.2 Hz, 1H), 6.92 (s, 1H), 5.40-5.26 (m, 1H), 3.07-2.97 (m, 1H), 2.93-2.81 (m, 1H), 2.80-2.71 (m, 1H), 2.69-2.60 (m, 2H), 2.13-2.03 (m, 1H), 1.98-1.89 (m, 1H), 1.85-1.71 (m, 1H), 0.86-0.80 (m, 4H). ¹⁹F NMR (376 MHz, DMSO-d₆) δ −110.39-−111.96 (m, 1F), −115.86-−118.57 (m, 1F). In addition, 1-(4-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-1-methyl-1H-pyrazol-3-yl)-2,2,2-trifluoroethan-1-one was also isolated from the reaction mixture and is characterized as Example 121.

### Example 16: (S)—N-(5-(2-cyano-5-((1-methylpyrrolidin-3-yl)methoxy)pyridin-4-yl)pyrazolo[1,5-a]pyridin-2-yl)cyclopropanecarboxamide

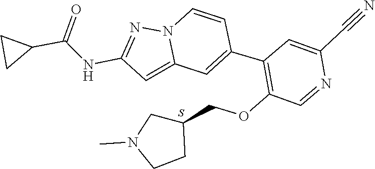

The title compound (38 mg, 77%) was prepared as described in Example 8 where (S)-(1-methylpyrrolidin-3-yl)methanol was used instead of (2S,4R)-tert-butyl 4-hydroxy-2-(trifluoromethyl)piperidine-1-carboxylate. MS (ESI): mass calcd. for C₂₃H₂₄N₆O₂, 416.20. m/z found, 417.2 [M+H]⁺. ¹H NMR (400 MHz, DMSO-d₆) δ 11.11 (s, 1H), 8.65 (s, 1H), 8.61 (dd, J=7.3, 1.0 Hz, 1H), 8.18 (s, 1H), 7.93 (dd, J=2.0, 0.9 Hz, 1H), 7.10 (dd, J=7.3, 2.1 Hz, 1H), 6.91 (s, 1H), 4.21 (d, J=6.7 Hz, 2H), 2.62-2.52 (m, 1H), 2.45-2.25 (m, 3H), 2.20 (s, 3H), 1.97-1.84 (m, 2H), 1.60-1.45 (m, 1H), 0.88-0.74 (m, 4H).

### Example 17: N-[5-[5-[[(1*S,4*S,5*R)-2-azabicyclo[2.2.1]heptan-5-yl]oxy]-2-cyano-4-pyridyl]pyrazolo[1,5-a]pyridin-2-yl]cyclopropanecarboxamide

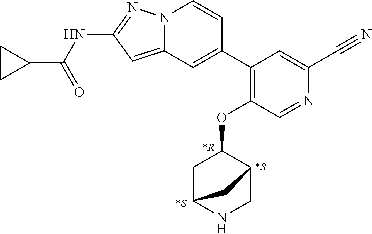 [SMILES: N#Cc1cc(-c2ccn3nc(NC(=O)C4CC4)cc3c2)c(O[C@@H]2C[C@@H]3C[C@H]2CN3)cn1]

The title compound (14.1 mg, 14%) was prepared as described in Example 8 where (*S,*S,*R)-tert-butyl 5-hydroxy-2-azabicyclo[2.2.1]heptane-2-carboxylate was used instead of (2S,4R)-tert-butyl 4-hydroxy-2-(trifluoromethyl)piperidine-1-carboxylate. MS (ESI): mass calcd. for C₂₃H₂₂N₆O₂, 414.2. m/z found, 415.2 [M+H]⁺. ¹H NMR (400 MHz, CD₃OD) δ 8.53 (s, 1H), 8.43 (d, J=7.2 Hz, 1H), 7.98 (s, 1H), 7.78 (d, J=0.8 Hz, 1H), 7.03 (dd, J=2.0, 7.6 Hz, 1H), 6.93 (s, 1H), 4.82-4.78 (m, 1H), 3.72-3.62 (m, 1H), 3.00-2.88 (m, 1H), 2.81-2.76 (m, 1H), 2.76-2.66 (m, 1H), 2.30-2.18 (m, 1H), 1.96-1.80 (m, 2H), 1.78-1.69 (m, 1H), 1.60-1.52 (m, 1H), 1.06-0.95 (m, 2H), 0.94-0.85 (m, 2H).

### Example 18: N-[5-[5-[[(1*R,4*R,5*S)-2-azabicyclo[2.2.1]heptan-5-yl]oxy]-2-cyano-4-pyridyl]pyrazolo[1,5-a]pyridin-2-yl]cyclopropanecarboxamide

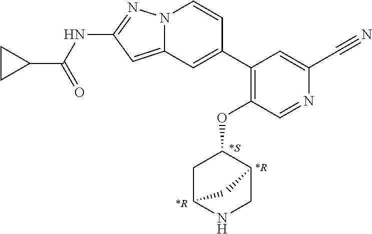 [SMILES: N#Cc1cc(-c2ccn3nc(NC(=O)C4CC4)cc3c2)c(O[C@H]2C[C@H]3C[C@@H]2CN3)cn1]

The title compound (10.2 mg, 10%) was prepared as described in Example 8 where (*R,*R,*S)-tert-butyl 5-hydroxy-2-azabicyclo[2.2.1]heptane-2-carboxylate was used instead of (2S,4R)-tert-butyl 4-hydroxy-2-(trifluoromethyl)piperidine-1-carboxylate. MS (ESI): mass calcd. for C₂₃H₂₂N₆O₂, 414.2. m/z found, 415.2 [M+H]⁺. ¹H NMR (400 MHz, CD₃OD) δ 8.53 (s, 1H), 8.43 (d, J=7.2 Hz, 1H), 7.97 (s, 1H), 7.78 (d, J=1.2 Hz, 1H), 7.03 (dd, J=2.0, 7.6 Hz, 1H), 6.93 (s, 1H), 4.82-4.78 (m, 1H), 3.73-3.61 (m, 1H), 2.99-2.92 (m, 1H), 2.79-2.74 (m, 1H), 2.73-2.67 (m, 1H), 2.29-2.19 (m, 1H), 1.91-1.80 (m, 2H), 1.78-1.71 (m, 1H), 1.60-1.54 (m, 1H), 1.03-0.97 (m, 2H), 0.93-0.87 (m, 2H).

### Example 19: N-[5-[2-cyano-5-[[(2S)-morpholin-2-yl]methoxy]-4-pyridyl]pyrazolo[1,5-a]pyridin-2-yl]cyclopropanecarboxamide

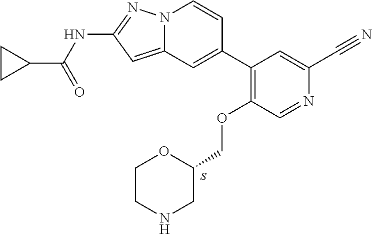

The title compound (76 mg, 31%) was prepared as described in Example 8 where (S)-tert-butyl 2-(hydroxymethyl)morpholine-4-carboxylate was used instead of (2S,4R)-tert-butyl 4-hydroxy-2-(trifluoromethyl)piperidine-1-carboxylate and N-(5-(5-chloro-2-cyanopyridin-4-yl)pyrazolo[1,5-a]pyridin-2-yl)cyclopropanecarboxamide was used instead of N-(5-(2-cyano-5-fluoropyridin-4-yl)pyrazolo[1,5-a]pyridin-2-yl)cyclopropanecarboxamide. MS (ESI): mass calcd. for C₂₂H₂₂N₆O₃, 418.2. m/z found, 419.1 [M+H]⁺. ¹H NMR (400 MHz, DMSO-d₆) δ 11.15 (s, 1H), 8.66 (s, 1H), 8.63 (d, J=7.2 Hz, 1H), 8.20 (s, 1H), 8.02 (d, J=1.2 Hz, 1H), 7.16 (dd, J=2.0, 7.2 Hz, 1H), 6.92 (s, 1H), 4.35-4.26 (m, 2H), 3.81-3.68 (m, 2H), 3.55-3.45 (m, 1H), 2.85-2.78 (m, 1H), 2.72-2.55 (m, 3H), 1.98-1.89 (m, 1H), 0.89-0.78 (m, 4H).

### Example 20: N-[5-[5-[[(1R,3S)-3-aminocyclopentyl]methoxy]-2-cyano-4-pyridyl]pyrazolo[1,5-a]pyridin-2-yl]cyclopropanecarboxamide

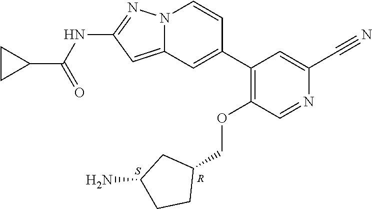

The title compound (11 mg, 8.8%) was prepared as described in Example 8 where tert-butyl ((1S,3R)-3-(hydroxymethyl)cyclopentyl)carbamate was used instead of (2S,4R)-tert-butyl 4-hydroxy-2-(trifluoromethyl)piperidine-1-carboxylate and N-(5-(5-chloro-2-cyanopyridin-4-yl)pyrazolo[1,5-a]pyridin-2-yl)cyclopropanecarboxamide was used instead of N-(5-(2-cyano-5-fluoropyridin-4-yl)pyrazolo[1,5-a]pyridin-2-yl)cyclopropanecarboxamide. MS (ESI): mass calcd. for C₂₃H₂₄N₆O₂, 416.2. m/z found, 417.2 [M+H]⁺. ¹H NMR (400 MHz, DMSO-d₆) δ 11.14 (s, 1H), 8.66 (s, 1H), 8.63 (d, J=7.2 Hz, 1H), 8.18 (s, 1H), 7.93 (s, 1H), 7.11 (dd, J=2.0, 7.2 Hz, 1H), 6.92 (s, 1H), 4.23 (d, J=7.2 Hz, 2H), 3.22-3.20 (m, 1H), 2.41-2.26 (m, 1H), 2.03-1.89 (m, 2H), 1.75-1.64 (m, 2H), 1.58-1.47 (m, 1H), 1.34-1.19 (m, 2H), 1.13-1.01 (m, 1H), 0.88-0.78 (m, 4H).

### Example 21: N-[5-[2-cyano-5-[[(6R)-1,4-oxazepan-6-yl]oxy]-4-pyridyl]pyrazolo[1,5-a]pyridin-2-yl]cyclopropanecarboxamide

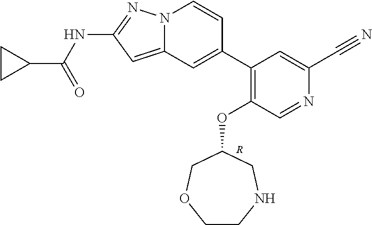

The title compound (30 mg, 13%) was prepared as described in Example 8 where (R)-tert-butyl 6-hydroxy-1,4-oxazepane-4-carboxylate was used instead of (2S,4R)-tert-butyl 4-hydroxy-2-(trifluoromethyl)piperidine-1-carboxylate. MS (ESI): mass calcd. for C₂₂H₂₂N₆O₃, 418.2. m/z found, 419.2 [M+H]⁺. ¹H NMR (400 MHz, DMSO-d₆) δ 11.14 (s, 1H), 8.64 (s, 1H), 8.62 (d, J=7.2 Hz, 1H), 8.18 (s, 1H), 7.98 (s, 1H), 7.16 (dd, J=2.0, 7.2 Hz, 1H), 6.92 (s, 1H), 5.10-5.03 (m, 1H), 4.05-3.97 (m, 1H), 3.94-3.85 (m, 1H), 3.75-3.66 (m, 1H), 3.58-3.49 (m, 1H), 3.29-3.23 (m, 1H), 2.89 (dd, J=6.0, 14.0 Hz, 1H), 2.85-2.73 (m, 2H), 1.98-1.88 (m, 1H), 0.87-0.78 (m, 4H).

### Example 22: N-[5-[5-[[(1R,3R)-3-aminocyclopentyl]methoxy]-2-cyano-4-pyridyl]pyrazolo[1,5-a]pyridin-2-yl]cyclopropanecarboxamide

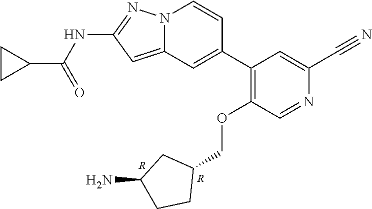

The title compound (4.3 mg, 7.5%) was prepared as described in Example 8 where tert-butyl ((1R,3R)-3-(hydroxymethyl)cyclopentyl)carbamate was used instead of (2S,4R)-tert-butyl 4-hydroxy-2-(trifluoromethyl)piperidine-1-carboxylate. MS (ESI): mass calcd. for C₂₃H₂₄N₆O₂, 416.2. m/z found, 417.3 [M+H]⁺. ¹H NMR (400 MHz, DMSO-d₆) δ 11.14 (s, 1H), 8.65 (s, 1H), 8.63 (d, J=7.2 Hz, 1H), 8.18 (s, 1H), 7.92 (d, J=0.8 Hz, 1H), 7.10 (dd, J=2.0, 7.2 Hz, 1H), 6.92 (s, 1H), 4.23-4.14 (m, 2H), 3.29-3.28 (m, 1H), 2.59-2.53 (m, 1H), 2.01-1.84 (m, 2H), 1.79-1.69 (m, 1H), 1.60-1.42 (m, 2H), 1.39-1.27 (m, 2H), 0.87-0.78 (m, 4H).

### Example 23: N-[5-[5-(azetidin-3-ylmethoxy)-2-cyano-4-pyridyl]pyrazolo[1,5-a]pyridin-2-yl]cyclopropanecarboxamide

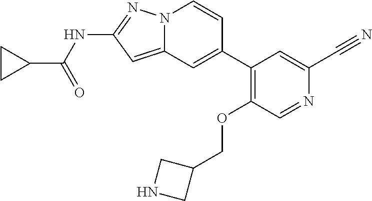 [SMILES: N#Cc1cc(-c2ccn3nc(NC(=O)C4CC4)cc3c2)c(OCC2CNC2)cn1]

The title compound (8.6 mg, 2.7%) was prepared as described in Example 8 where tert-butyl 3-(hydroxymethyl)azetidine-1-carboxylate was used instead of (2S,4R)-tert-butyl 4-hydroxy-2-(trifluoromethyl)piperidine-1-carboxylate. MS (ESI): mass calcd. for C₂₁H₂₀N₆O₂, 388.2. m/z found, 389.1 [M+H]⁺. ¹H NMR (400 MHz, DMSO-d₆) δ 11.14 (s, 1H), 8.69 (s, 1H), 8.61 (d, J=7.2 Hz, 1H), 8.20 (s, 1H), 7.95-7.89 (m, 1H), 7.10 (dd, J=2.0, 7.2 Hz, 1H), 6.92 (s, 1H), 4.45 (d, J=6.4 Hz, 2H), 3.57-3.48 (m, 2H), 3.37-3.33 (m, 2H), 3.11-2.96 (m, 1H), 1.98-1.89 (m, 1H), 0.86-0.79 (m, 4H).

### Example 24: N-[5-[5-[[(1R,4R,5R)-2-azabicyclo[2.2.1]heptan-5-yl]oxy]-2-cyano-4-pyridyl]pyrazolo[1,5-a]pyridin-2-yl]cyclopropanecarboxamide

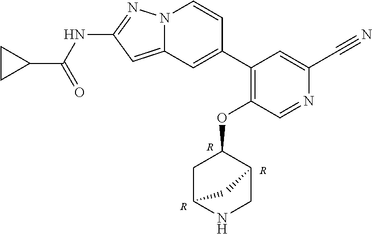

The title compound (150 mg, 7.4%) was prepared as described in Example 8 where (1R,4R,5R)-tert-butyl 5-hydroxy-2-azabicyclo[2.2.1]heptane-2-carboxylate was used instead of (2S,4R)-tert-butyl 4-hydroxy-2-(trifluoromethyl)piperidine-1-carboxylate. MS (ESI): mass calcd. for C₂₃H₂₂N₆O₂, 414.2. m/z found, 415.2 [M+H]⁺. ¹H NMR (400 MHz, CD₃OD) δ 8.48-8.41 (m, 2H), 7.97 (s, 1H), 7.80 (s, 1H), 7.06 (dd, J=2.0, 6.8 Hz, 1H), 6.95 (s, 1H), 5.18-5.03 (m, 1H), 3.70-3.59 (m, 1H), 3.26-3.20 (m, 1H), 2.94-2.86 (m, 1H), 2.84-2.77 (m, 1H), 2.43-2.29 (m, 1H), 1.91-1.83 (m, 1H), 1.82-1.77 (m, 1H), 1.75-1.69 (m, 1H), 1.52-1.42 (m, 1H), 1.03-0.96 (m, 2H), 0.93-0.86 (m, 2H).

### Example 25: N-[5-[2-cyano-5-[(3R)-1-cyclopropylpyrrolidin-3-yl]oxy-4-pyridyl]pyrazolo[1,5-a]pyridin-2-yl]cyclopropanecarboxamide

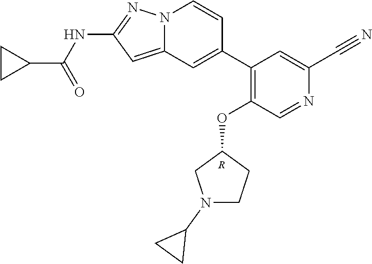

The title compound (45 mg, 39%) was prepared as described in Example 7 (using (R)-pyrrolidin-3-ol instead of (S)-pyrrolidin-3-ol) and. MS (ESI): mass calcd. for C₂₄H₂₄N₆O₂, 428.2. m/z found, 429.3 [M+H]⁺. ¹H NMR (400 MHz, DMSO-d₆) δ 11.14 (s, 1H), 8.64-8.58 (m, 2H), 8.19 (s, 1H), 7.95 (d, J=1.2 Hz, 1H), 7.12 (dd, J=2.0, 7.2 Hz, 1H), 6.91 (s, 1H), 5.29-5.23 (m, 1H), 3.08-3.01 (m, 1H), 2.87-2.79 (m, 2H), 2.65-2.55 (m, 1H), 2.39-2.28 (m, 1H), 1.99-1.89 (m, 1H), 1.88-1.78 (m, 1H), 1.72-1.64 (m, 1H), 0.86-0.80 (m, 4H), 0.41-0.36 (m, 2H), 0.36-0.31 (m, 1H), 0.28-0.23 (m, 1H).

### Example 26: N-[5-[2-cyano-5-[[(6S)-4-methyl-1,4-oxazepan-6-yl]oxy]-4-pyridyl]pyrazolo[1,5-a]pyridin-2-yl]cyclopropanecarboxamide

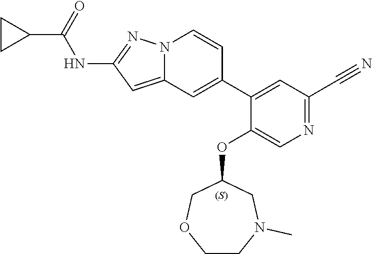

The title compound (26 mg, 72%) was prepared as described in Example 7 where paraformaldehyde was used instead of (1-ethoxycyclopropoxy)trimethylsilane and (S)-1,4-oxazepan-6-ol was used instead of (S)-pyrrolidin-3-ol. MS (ESI): mass calcd. for C₂₃H₂₄N₆O₃, 432.2. m/z found, 433.2 [M+H]⁺. ¹H NMR (400 MHz, DMSO-d₆) δ 11.15 (s, 1H), 8.65 (s, 1H), 8.62 (d, J=7.2 Hz, 1H), 8.19 (s, 1H), 8.00 (d, J=1.2 Hz, 1H), 7.17 (dd, J=2.0, 7.2 Hz, 1H), 6.91 (s, 1H), 5.16-5.09 (m, 1H), 4.09-4.02 (m, 1H), 3.89-3.82 (m, 1H), 3.74-3.67 (m, 1H), 3.64-3.56 (m, 1H), 3.05-2.97 (m, 1H), 2.81-2.73 (m, 1H), 2.62-2.56 (m, 2H), 2.31 (s, 3H), 1.98-1.89 (m, 1H), 0.86-0.80 (m, 4H).

### Example 27: N-[5-[2-cyano-5-[[(1S,5R)-8-methyl-8-azabicyclo[3.2.1]octan-3-yl]methoxy]-4-pyridyl]pyrazolo[1,5-a]pyridin-2-yl]cyclopropanecarboxamide

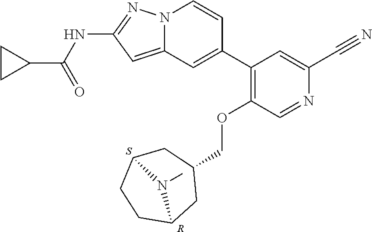

The title compound (9.5 mg, 22%) was prepared as described in Example 7 where paraformaldehyde was used instead of (1-ethoxycyclopropoxy)trimethylsilane and (1R,3s,5S)-8-azabicyclo[3.2.1]octan-3-ylmethanol hydrochloride was used instead of (S)-pyrrolidin-3-ol. MS (ESI): mass calcd. for C₂₆H₂₈N₆O₂, 456.2. m/z found, 457.3 [M+H]⁺. ¹H NMR (400 MHz, DMSO-d₆) δ 11.14 (s, 1H), 8.65 (s, 1H), 8.62 (d, J=7.2 Hz, 1H), 8.17 (s, 1H), 7.96-7.87 (m, 1H), 7.09 (dd, J=2.0, 7.6 Hz, 1H), 6.93 (s, 1H), 4.12 (d, J=6.4 Hz, 2H), 3.37 (s, 2H), 2.32 (s, 3H), 2.27-2.12 (m, 1H), 2.09-1.88 (m, 3H), 1.68-1.61 (m, 2H), 1.60-1.53 (m, 3H), 1.26-1.22 (m, 1H), 0.89-0.78 (m, 4H).

### Example 28: N-[5-[2-cyano-5-[[(3S,5R)-1-methyl-5-(trifluoromethyl)-3-piperidyl]oxy]-4-pyridyl]pyrazolo[1,5-a]pyridin-2-yl]cyclopropanecarboxamide

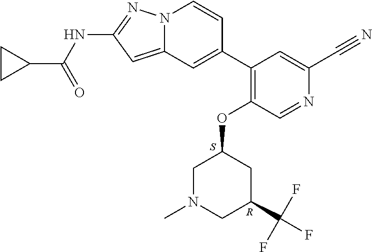

The title compound (15 mg, 29%) was prepared as described in Example 7 where was paraformaldehyde used instead of (1-ethoxycyclopropoxy)trimethylsilane and N-(5-(2-cyano-5-chloropyridin-4-yl)pyrazolo[1,5-a]pyridin-2-yl)cyclopropanecarboxamide was used instead of N-(5-(2-cyano-5-fluoropyridin-4-yl)pyrazolo[1,5-a]pyridin-2-yl)cyclopropanecarboxamide and (3S,5R)-5-(trifluoromethyl)piperidin-3-ol was used instead of (S)-pyrrolidin-3-ol. MS (ESI): mass calcd. for C₂₄H₂₃F₃N₆O₂, 484.2. m/z found, 485.2 [M+H]⁺. ¹H NMR (400 MHz, DMSO-d₆) δ 11.14 (s, 1H), 8.84 (s, 1H), 8.61 (d, J=7.2 Hz, 1H), 8.20 (s, 1H), 7.90 (d, J=0.8 Hz, 1H), 7.08 (dd, J=1.6, 7.2 Hz, 1H), 6.94 (s, 1H), 4.95-4.82 (m, 1H), 3.23-3.12 (m, 1H), 2.98-2.88 (m, 1H), 2.86-2.72 (m, 1H), 2.46-2.40 (m, 1H), 2.27 (s, 3H), 2.02-1.86 (m, 3H), 1.37-1.24 (m, 1H), 0.87-0.79 (m, 4H). 19F NMR (376 MHz, DMSO-d₆) δ −70.58 (d, J=8.6 Hz, 3F).

### Example 29: N-(5-(2-cyano-5-(((2*S,4*R)-1-methyl-2-(trifluoromethyl)piperidin-4-yl)oxy)pyridin-4-yl)pyrazolo[1,5-a]pyridin-2-yl)cyclopropanecarboxamide

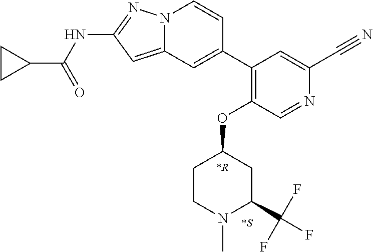 [SMILES: CN1CC[C@@H](Oc2cnc(C#N)cc2-c2ccn3nc(NC(=O)C4CC4)cc3c2)C[C@H]1C(F)(F)F]

The title compound (11.6 mg, 13%) was prepared as described in Example 7 where paraformaldehyde was used instead of (1-ethoxycyclopropoxy)trimethylsilane and N-(5-(2-cyano-5-chloropyridin-4-yl)pyrazolo[1,5-a]pyridin-2-yl)cyclopropanecarboxamide was used instead of N-(5-(2-cyano-5-fluoropyridin-4-yl)pyrazolo[1,5-a]pyridin-2-yl)cyclopropanecarboxamide and rac-5-(trifluoromethyl)piperidin-3-ol was used instead of (S)-pyrrolidin-3-ol. The racemic mixture was purified by SFC over DAICEL CHIRALPAK AD 250 mm×30 mm, 10 μm (eluent: 40% to 40% (v/v) 0.1% NH₃—H₂O EtOH) to yield the title compound as the first eluting peak. MS (ESI): mass calcd. for C₂₄H₂₃F₃N₆O₂, 484.2. m/z found, 485.2 [M+H]⁺. ¹H NMR (400 MHz, DMSO-d₆) δ 11.12 (s, 1H), 8.79 (s, 1H), 8.59 (d, J=7.2 Hz, 1H), 8.18 (s, 1H), 7.90 (d, J=1.2 Hz, 1H), 7.08 (dd, J=2.0, 6.8 Hz, 1H), 6.93 (s, 1H), 4.91-4.81 (m, 1H), 3.12-3.00 (m, 1H), 2.96-2.86 (m, 1H), 2.47-2.40 (m, 1H), 2.33-2.25 (m, 4H), 2.12-2.03 (m, 1H), 1.97-1.88 (m, 1H), 1.71-1.52 (m, 2H), 0.85-0.79 (m, 4H). ¹⁹F NMR (376 MHz, DMSO-d₆) δ −69.00 (d, J=7.1 Hz, 3F).

### Example 30: N-(5-(2-cyano-5-(((2*R,4*S)-1-methyl-2-(trifluoromethyl)piperidin-4-yl)oxy)pyridin-4-yl)pyrazolo[1,5-a]pyridin-2-yl)cyclopropanecarboxamide

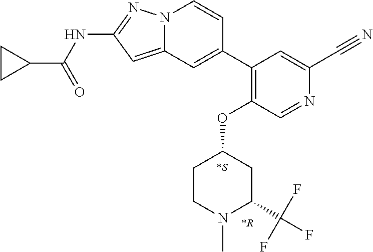 [SMILES: CN1CC[C@H](Oc2cnc(C#N)cc2-c2ccn3nc(NC(=O)C4CC4)cc3c2)C[C@@H]1C(F)(F)F]

The title compound (8.3 mg, 9%) was prepared as described in Example 7 where paraformaldehyde was used instead of (1-ethoxycyclopropoxy)trimethylsilane and N-(5-(2-cyano-5-chloropyridin-4-yl)pyrazolo[1,5-a]pyridin-2-yl)cyclopropanecarboxamide was used instead of N-(5-(2-cyano-5-fluoropyridin-4-yl)pyrazolo[1,5-a]pyridin-2-yl)cyclopropanecarboxamide and rac-5-(trifluoromethyl)piperidin-3-ol was used instead of (S)-pyrrolidin-3-ol. The racemic mixture was purified by SFC over DAICEL CHIRALPAK AD 250 mm×30 mm, 10 μm (eluent: 40% to 40% (v/v) 0.1% NH₃—H₂O ETOH) to yield the title compound as the second eluting peak MS (ESI): mass calcd. for C₂₄H₂₃F₃N₆O₂, 484.2. m/z found, 485.2 [M+H]⁺. ¹H NMR (400 MHz, DMSO-d₆) δ 11.14 (s, 1H), 8.80 (s, 1H), 8.60 (d, J=7.2 Hz, 1H), 8.19 (s, 1H), 7.91 (d, J=0.8 Hz, 1H), 7.09 (dd, J=2.0, 6.8 Hz, 1H), 6.93 (s, 1H), 4.92-4.81 (m, 1H), 3.14-3.01 (m, 1H), 2.96-2.89 (m, 1H), 2.48-2.40 (m, 1H), 2.33-2.25 (m, 4H), 2.13-2.05 (m, 1H), 1.97-1.88 (m, 1H), 1.70-1.53 (m, 2H), 0.87-0.79 (m, 4H). ¹⁹F NMR (376 MHz, DMSO-d₆) δ −68.98 (d, J=6.8 Hz, 3F).

### Example 31: N-[5-[5-(azetidin-3-yloxy)-2-cyano-4-pyridyl]pyrazolo[1,5-a]pyridin-2-yl]cyclopropanecarboxamide

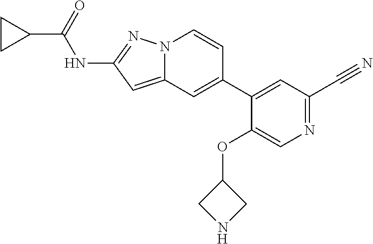

The title compound (9.0 mg, 14%) was prepared as described in Example 1 where azetidin-3-ol was used instead of (R)-(1-methylpyrrolidin-3-yl)methanol. MS (ESI): mass calcd. for C₂₀H₁₈N₆O₂, 374.1. m/z found, 375.2 [M+H]⁺. ¹H NMR (400 MHz, DMSO-d₆) δ11.12 (d, J=2.9 Hz, 1H), 8.66-8.57 (m, 1H), 8.39 (s, OH), 8.33 (s, 1H), 8.21 (d, J=4.0 Hz, 1H), 7.98-7.92 (m, 1H), 7.18-7.09 (m, 1H), 6.94 (d, J=5.0 Hz, 1H), 5.34 (p, J=5.9 Hz, 1H), 5.18 (p, J=11.0, 5.2 Hz, OH), 3.88-3.80 (m, 1H), 3.80-3.72 (m, 1H), 3.55 (dd, J=9.3, 5.5 Hz, 1H), 3.18 (dd, J=8.5, 5.1 Hz, 1H), 1.95 (d, J=4.8 Hz, 1H), 0.90-0.75 (m, 4H).

### Example 32: N-[5-[2-cyano-5-[(1-methylazetidin-3-yl)methoxy]-4-pyridyl]pyrazolo[1,5-a]pyridin-2-yl]cyclopropanecarboxamide

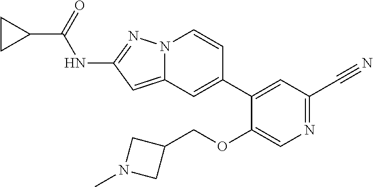 [SMILES: CN1CC(COc2cnc(C#N)cc2-c2ccn3nc(NC(=O)C4CC4)cc3c2)C1]

The title compound (22 mg, 28%) was prepared as described in Example 1 where (1-methylazetidin-3-yl)methanol was used instead of (R)-(1-methylpyrrolidin-3-yl)methanol. MS (ESI): mass calcd. for C₂₂H₂₂N₆O₂, 402.2. m/z found, 403.2 [M+H]⁺. ¹H NMR (400 MHz, DMSO-d₆) δ 11.15 (s, 1H), 8.67 (s, 1H), 8.63 (d, J=7.3 Hz, 1H), 8.19 (s, 1H), 7.94 (d, J=0.8 Hz, 1H), 7.09 (dd, J=1.8, 7.3 Hz, 1H), 6.92 (s, 1H), 4.41 (d, J=6.4 Hz, 2H), 3.30-3.26 (m, 2H), 3.02-2.96 (m, 2H), 2.84-2.75 (m, 1H), 2.15 (s, 3H), 1.98-1.89 (m, 1H), 0.86-0.79 (m, 4H).

### Example 33: rac-N-(5-(2-cyano-5-((5-oxopyrrolidin-3-yl)methoxy)pyridin-4-yl)pyrazolo[1,5-a]pyridin-2-yl)cyclopropanecarboxamide

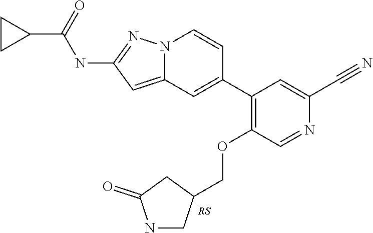 [SMILES: N#Cc1cc(-c2ccn3nc(NC(=O)C4CC4)cc3c2)c(OCC2CNC(=O)C2)cn1]

The title compound (91 mg, 71%) was prepared as described in Example 1 where 4-(hydroxymethyl)pyrrolidin-2-one was used instead of (R)-(1-methylpyrrolidin-3-yl)methanol. MS (ESI): mass calcd. for C₂₂H₂₀N₆O₃, 416.2. m/z found, 417.3 [M+H]⁺. ¹H NMR (400 MHz, DMSO-d₆) δ 11.11 (s, 1H), 8.67 (s, 1H), 8.59 (dd, J=7.3, 1.0 Hz, 1H), 8.19 (s, 1H), 7.90 (dd, J=2.0, 0.9 Hz, 1H), 7.54 (s, 1H), 7.09 (dd, J=7.3, 2.0 Hz, 1H), 6.93 (s, 1H), 4.37-4.24 (m, 2H), 3.40-3.32 (m, 1H), 3.10 (dd, J=9.8, 5.8 Hz, 1H), 2.94-2.79 (m, 1H), 2.30 (dd, J=16.7, 9.2 Hz, 1H), 2.06 (dd, J=16.7, 7.0 Hz, 1H), 1.98-1.89 (m, 1H), 0.89-0.76 (m, 4H).

### Example 34: tert-butyl 2-[[6-cyano-4-[2-(cyclopropanecarbonylamino)pyrazolo[1,5-a]pyridin-5-yl]-3-pyridyl]oxy]-6-azaspiro[3.3]heptane-6-carboxylate

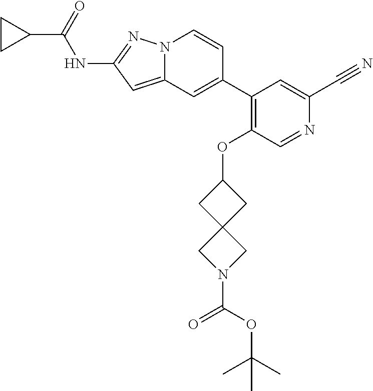 [SMILES: CC(C)(C)OC(=O)N1CC2(CC(Oc3cnc(C#N)cc3-c3ccn4nc(NC(=O)C5CC5)cc4c3)C2)C1]

The title compound (125 mg, 52%) was prepared as described in Example 1 where tert-butyl 6-hydroxy-2-azaspiro[3.3]heptane-2-carboxylate was used instead of (R)-(1-methylpyrrolidin-3-yl)methanol. MS (ESI): mass calcd. for C₂₈H₃₀N₆O₄, 514.2. m/z found, 415.2 [M+H]⁺. ¹H NMR (400 MHz, DMSO-d₆) δ 11.11 (s, 1H), 8.64-8.57 (m, 1H), 8.45 (s, 1H), 8.19 (s, 1H), 7.95-7.88 (m, 1H), 7.09 (dd, J=7.3, 2.0 Hz, 1H), 6.96-6.91 (m, 1H), 5.04-4.90 (m, 1H), 3.86 (d, J=38.3 Hz, 4H), 2.84-2.74 (m, 2H), 2.35-2.22 (m, 2H), 2.01-1.88 (m, 1H), 1.36 (s, 9H), 0.94-0.76 (m, 4H).

### Example 35: tert-butyl (3R)-3-[[6-cyano-4-[2-(cyclopropanecarbonylamino)pyrazolo[1,5-a]pyridin-5-yl]-3-pyridyl]oxymethyl]pyrrolidine-1-carboxylate

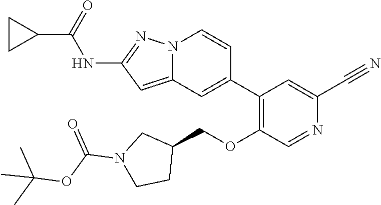 [SMILES: CC(C)(C)OC(=O)N1CC[C@H](COc2cnc(C#N)cc2-c2ccn3nc(NC(=O)C4CC4)cc3c2)C1]

The title compound (2020 mg, 60%) was prepared as described in Example 1 where tert-butyl (R)-3-(hydroxymethyl)pyrrolidine-1-carboxylate was used instead of (R)-(1-methylpyrrolidin-3-yl)methanol. MS (ESI): mass calcd. for C₂₇H₃₀N₆O₄, 502.2. m/z found, 447.2 [M+H]⁺. ¹H NMR (400 MHz, DMSO-d₆) δ 8.67 (s, 1H), 8.63-8.57 (m, 1H), 8.19 (s, 1H), 7.98-7.88 (m, 2H), 7.09 (dd, J=7.3, 2.0 Hz, 1H), 6.93 (s, 1H), 4.42-4.21 (m, 2H), 3.46-3.33 (m, 2H), 3.26-3.15 (m, 1H), 3.10-2.98 (m, 1H), 2.69-2.60 (m, 1H), 2.01-1.89 (m, 2H), 1.77-1.60 (m, 1H), 1.36 (d, J=10.5 Hz, 9H), 0.87-0.75 (m, 4H).

### Example 36: (R)—N-(5-(2-cyano-5-((4-methylmorpholin-2-yl)methoxy)pyridin-4-yl)pyrazolo[1,5-a]pyridin-2-yl)cyclopropanecarboxamide

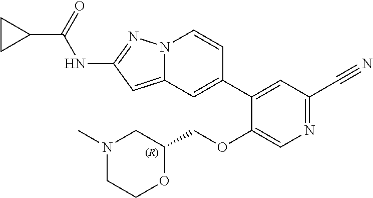

The title compound (105 mg, 54%) was prepared as described in Example 1 where (R)-(4-methylmorpholin-2-yl)methanol was used instead of (R)-(1-methylpyrrolidin-3-yl)methanol. MS (ESI): mass calcd. for C₂₃H₂₄N₆O₃, 432.2. m/z found, 433.2 [M+H]⁺. ¹H NMR (400 MHz, DMSO-d₆) δ 11.13 (s, 1H), 8.67 (s, 1H), 8.63 (d, J=7.2 Hz, 1H), 8.20 (s, 1H), 8.01 (d, J=1.2 Hz, 1H), 7.16 (dd, J=2.0, 7.6 Hz, 1H), 6.92 (s, 1H), 4.41-4.30 (m, 2H), 3.85-3.78 (m, 2H), 3.58-3.49 (m, 1H), 2.76-2.70 (m, 1H), 2.63-2.56 (m, 1H), 2.15 (s, 3H), 1.99-1.86 (m, 3H), 0.86-0.80 (m, 4H).

### Example 37: (R)—N-(5-(2-cyano-5-((1-methylpiperidin-3-yl)methoxy)pyridin-4-yl)pyrazolo[1,5-a]pyridin-2-yl)cyclopropanecarboxamide

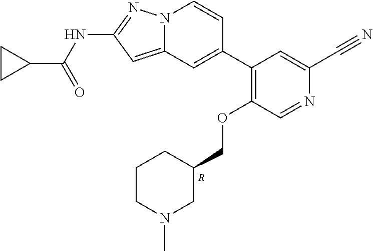

The title compound (50 mg, 25%) was prepared as described in Example 1 where (R)-(1-methylpiperidin-3-yl)methanol was used instead of (R)-(1-methylpyrrolidin-3-yl)methanol. MS (ESI): mass calcd. for C₂₄H₂₆N₆O₂, 430.2. m/z found, 431.2 [M+H]⁺. ¹H NMR (400 MHz, DMSO-d₆) δ 11.13 (s, 1H), 8.68-8.61 (m, 2H), 8.18 (s, 1H), 7.91 (d, J=1.2 Hz, 1H), 7.08 (dd, J=7.2, 2.0 Hz, 1H), 6.92 (s, 1H), 4.25-4.14 (m, 2H), 2.76-2.66 (m, 1H), 2.62-2.54 (m, 1H), 2.11 (s, 3H), 2.03-1.77 (m, 4H), 1.71-1.57 (m, 2H), 1.52-1.36 (m, 1H), 1.12-1.00 (m, 1H), 0.87-0.79 (m, 4H).

### Example 38: (S)—N-(5-(2-cyano-5-((1-methylpiperidin-3-yl)methoxy)pyridin-4-yl)pyrazolo[1,5-a]pyridin-2-yl)cyclopropanecarboxamide

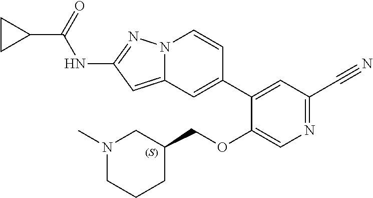

The title compound (16 mg, 19%) was prepared as described in Example 1 where (S)-(1-methylpiperidin-3-yl)methanol was used instead of (R)-(1-methylpyrrolidin-3-yl)methanol. MS (ESI): mass calcd. for C₂₄H₂₆N₆O₂, 430.2. m/z found, 431.2 [M+H]⁺. ¹H NMR (400 MHz, DMSO-d₆) δ 11.13 (s, 1H), 8.69-8.60 (m, 2H), 8.18 (s, 1H), 7.90 (s, 1H), 7.07 (dd, J=1.6, 7.2 Hz, 1H), 6.91 (s, 1H), 4.25-4.14 (m, 2H), 2.74-2.65 (m, 1H), 2.59-2.54 (m, 1H), 2.10 (s, 3H), 2.04-1.92 (m, 2H), 1.88-1.74 (m, 2H), 1.69-1.55 (m, 2H), 1.51-1.37 (m, 1H), 1.13-0.98 (m, 1H), 0.87-0.78 (m, 4H).

### Example 39: N-[5-[2-cyano-5-[[(2S)-4-methylmorpholin-2-yl]methoxy]-4-pyridyl]pyrazolo[1,5-a]pyridin-2-yl]cyclopropanecarboxamide

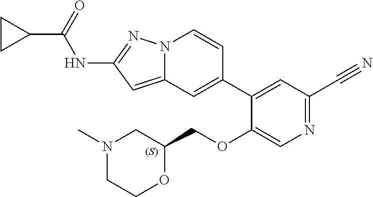

The title compound (16 mg, 19%) was prepared as described in Example 1 where (S)-(4-methylmorpholin-2-yl)methanol was used instead of (R)-(1-methylpyrrolidin-3-yl)methanol. MS (ESI): mass calcd. for C₂₃H₂₄N₆O₃, 432.2. m/z found, 433.1 [M+H]⁺. ¹H NMR (400 MHz, DMSO-d₆) δ 11.14 (s, 1H), 8.67 (s, 1H), 8.63 (d, J=7.2 Hz, 1H), 8.21 (s, 1H), 8.01 (d, J=1.2 Hz, 1H), 7.16 (dd, J=2.0, 7.2 Hz, 1H), 6.92 (s, 1H), 4.41-4.29 (m, 2H), 3.86-3.77 (m, 2H), 3.58-3.49 (m, 1H), 2.76-2.69 (m, 1H), 2.62-2.55 (m, 1H), 2.15 (s, 3H), 2.00-1.85 (m, 3H), 0.87-0.77 (m, 4H).

### Example 40: N-[5-[2-cyano-5-[(2-oxo-3-azabicyclo[2.2.2]octan-4-yl)methoxy]-4-pyridyl]pyrazolo[1,5-a]pyridin-2-yl]cyclopropanecarboxamide

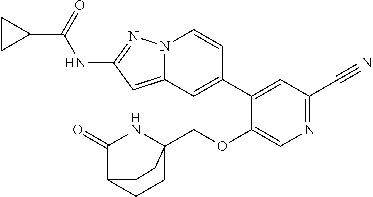

The title compound (22 mg, 23%) was prepared as described in Example 1 where (1s,4s)-1-(hydroxymethyl)-2-azabicyclo[2.2.2]octan-3-one was used instead of (R)-(1-methylpyrrolidin-3-yl)methanol. MS (ESI): mass calcd. for C₂₅H₂₄N₆O₃, 456.2. m/z found, 457.2 [M+H]⁺. ¹H NMR (400 MHz, DMSO-d₆) δ 11.12 (s, 1H), 8.65 (s, 1H), 8.60 (dd, J=7.2, 1.0 Hz, 1H), 8.20 (s, 1H), 8.12 (d, J=1.7 Hz, 1H), 8.03-7.98 (m, 1H), 7.18 (dd, J=7.3, 2.0 Hz, 1H), 6.87 (s, 1H), 4.29 (s, 2H), 2.31-2.18 (m, 1H), 1.93 (m, J=6.0, 5.0 Hz, 1H), 1.77-1.53 (m, 8H), 0.92-0.74 (m, 4H).

### Example 41: N-[5-[2-cyano-5-[(1-hydroxycyclobutyl)methoxy]-4-pyridyl]pyrazolo[1,5-a]pyridin-2-yl]cyclopropanecarboxamide

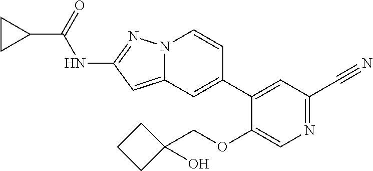 [SMILES: N#Cc1cc(-c2ccn3nc(NC(=O)C4CC4)cc3c2)c(OCC2(O)CCC2)cn1]

The title compound (33.5 mg, 32%) was prepared as described in Example 1 where 1-(hydroxymethyl)cyclobutan-1-ol was used instead of (R)-(1-methylpyrrolidin-3-yl)methanol. MS (ESI): mass calcd. for C₂₂H₂₁N₅O₃, 403.2. m/z found, 404.2 [M+H]⁺. ¹H NMR (400 MHz, DMSO-d₆) δ 11.11 (s, 1H), 8.71 (s, 1H), 8.59 (dt, J=7.3, 0.9 Hz, 1H), 8.19 (s, 1H), 8.05 (dd, J=2.0, 1.0 Hz, 1H), 7.18 (dd, J=7.3, 2.0 Hz, 1H), 6.90 (s, 1H), 5.38 (s, 1H), 4.29 (s, 2H), 2.17-1.88 (m, 5H), 1.71-1.59 (m, 1H), 1.59-1.44 (m, 1H), 0.90-0.73 (m, 4H).

### Example 42: N-[5-[2-cyano-5-[(1-hydroxycyclopentyl)methoxy]-4-pyridyl]pyrazolo[1,5-a]pyridin-2-yl]cyclopropanecarboxamide

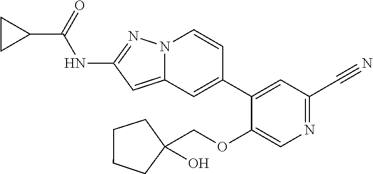 [SMILES: N#Cc1cc(-c2ccn3nc(NC(=O)C4CC4)cc3c2)c(OCC2(O)CCCC2)cn1]

The title compound (66 mg, 37%) was prepared as described in Example 1 where 1-(hydroxymethyl)cyclopentan-1-ol was used instead of (R)-(1-methylpyrrolidin-3-yl)methanol. MS (ESI): mass calcd. for C₂₃H₂₃N₅O₃, 417.2. m/z found, 418.1 [M+H]⁺. ¹H NMR (400 MHz, DMSO-d₆) δ 11.12 (s, 1H), 8.68 (s, 1H), 8.63-8.55 (m, 1H), 8.20 (s, 1H), 8.07-8.03 (m, 1H), 7.19 (dd, J=7.3, 2.1 Hz, 1H), 6.97-6.84 (m, 1H), 4.68 (s, 1H), 4.23 (s, 2H), 2.02-1.89 (m, 1H), 1.75-1.50 (m, 8H), 0.91-0.72 (m, 4H).

### Example 43: N-[5-[2-cyano-5-(4-piperidylmethoxy)-4-pyridyl]pyrazolo[1,5-a]pyridin-2-yl]cyclopropanecarboxamide

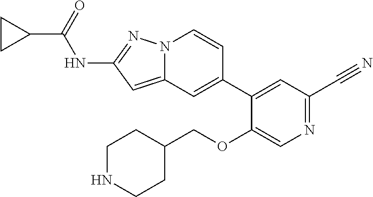 [SMILES: N#Cc1cc(-c2ccn3nc(NC(=O)C4CC4)cc3c2)c(OCC2CCNCC2)cn1]

The title compound (50 mg, 19%) was prepared as described in Example 1 where piperidin-4-ylmethanol was used instead of (R)-(1-methylpyrrolidin-3-yl)methanol. MS (ESI): mass calcd. for C₂₃H₂₄N₆O₂, 416.2. m/z found, 417.2 [M+H]⁺; ¹H NMR (400 MHz, DMSO-d₆) δ 11.15 (s, 1H), 8.67 (s, 1H), 8.63 (d, J=7.2 Hz, 1H), 8.17 (s, 1H), 7.90 (s, 1H), 7.09 (dd, J=7.2, 1.6 Hz, 1H), 6.92 (s, 1H), 4.15 (d, J=6.0 Hz, 2H), 2.99-2.85 (m, 2H), 2.48-2.38 (m, 2H), 2.00-1.90 (m, 1H), 1.89-1.77 (m, 1H), 1.70-1.56 (m, 2H), 1.24-1.07 (m, 2H), 0.90-0.76 (m, 4H).

### Example 44: N-[5-[5-[(1-aminocyclobutyl)methoxy]-2-cyano-4-pyridyl]pyrazolo[1,5-a]pyridin-2-yl]cyclopropanecarboxamide

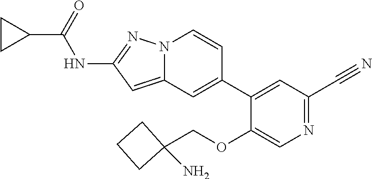 [SMILES: N#Cc1cc(-c2ccn3nc(NC(=O)C4CC4)cc3c2)c(OCC2(N)CCC2)cn1]

The title compound (50 mg, 19%) was prepared as described in Example 1 where (1-aminocyclobutyl)methanol was used instead of (R)-(1-methylpyrrolidin-3-yl)methanol. MS (ESI): mass calcd. for C₂₂H₂₂N₆O₂, 402.2. m/z found, 403.2 [M+H]⁺. ¹H NMR (600 MHz, DMSO-d₆) δ 11.10 (s, 1H), 8.70 (s, 1H), 8.64-8.57 (m, 1H), 8.19 (s, 1H), 8.05-7.96 (m, 1H), 7.15 (dd, J=7.3, 2.0 Hz, 1H), 6.90 (s, 1H), 4.20 (s, 2H), 2.08-2.01 (m, 2H), 1.97-1.90 (m, 1H), 1.89-1.76 (m, 4H), 1.76-1.67 (m, 1H), 1.66-1.56 (m, 1H), 0.86-0.79 (m, 4H).

### Example 45: N-[5-[2-Cyano-5-[[(3R)-pyrrolidin-3-yl]methoxy]-4-pyridyl]pyrazolo[1,5-a]pyridin-2-yl]cyclopropanecarboxamide

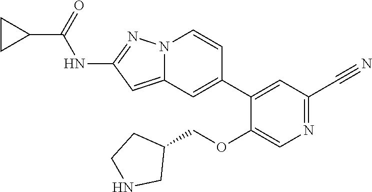 [SMILES: N#Cc1cc(-c2ccn3nc(NC(=O)C4CC4)cc3c2)c(OC[C@H]2CCNC2)cn1]

The title compound (151 mg, 63%) was prepared as described in Example 1 where (R)-pyrrolidin-3-ylmethanol was used instead of (R)-(1-methylpyrrolidin-3-yl)methanol. MS (ESI): mass calcd. for C₂₂H₂₂N₆O₂, 402.2. m/z found, 403.2 [M+H]⁺. ¹H NMR (400 MHz, DMSO-d₆) δ 11.12 (s, 1H), 8.69-8.59 (m, 2H), 8.18 (s, 1H), 7.94-7.89 (m, 1H), 7.09 (dd, J=7.3, 2.0 Hz, 1H), 6.91 (s, 1H), 4.32-4.15 (m, 2H), 2.84 (dd, J=10.9, 7.5 Hz, 1H), 2.81-2.64 (m, 2H), 2.57 (dd, J=10.9, 5.7 Hz, 1H), 2.46-2.37 (m, 1H), 1.99-1.88 (m, 1H), 1.87-1.71 (m, 1H), 1.47-1.35 (m, 1H), 0.91-0.73 (m, 5H).

### Example 46: N-[5-[2-Cyano-5-(cyclopropoxy)-4-pyridyl]pyrazolo[1,5-a]pyridin-2-yl]cyclopropanecarboxamide

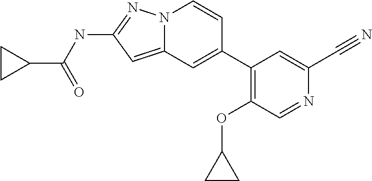 [SMILES: N#Cc1cc(-c2ccn3nc(NC(=O)C4CC4)cc3c2)c(OC2CC2)cn1]

The title compound (7.1 mg, 8%) was prepared as described in Example 1 where cyclopropanol was used instead of (R)-(1-methylpyrrolidin-3-yl)methanol. MS (ESI): mass calcd. for C₂₀H₁₇N₅O₂, 359.1. m/z found, 360.2 [M+H]⁺; ¹H NMR (400 MHz, DMSO-d₆) δ 11.12 (s, 1H), 8.93 (s, 1H), 8.59 (d, J=7.6 Hz, 1H), 8.18 (s, 1H), 7.86 (d, J=1.2 Hz, 1H), 7.03 (dd, J=7.2, 2.0, Hz 1H), 6.93 (s, 1H), 4.28-4.20 (m, 1H), 1.98-1.90 (m, 1H), 0.94-0.88 (m, 2H), 0.86-0.77 (m, 6H).

### Example 47: N-[5-[2-Cyano-5-(3-pyridylmethoxy)-4-pyridyl]pyrazolo[1,5-a]pyridin-2-yl]cyclopropanecarboxamide

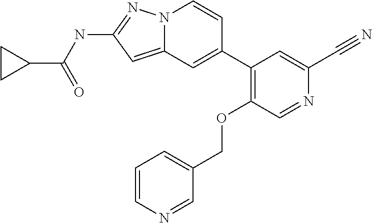 [SMILES: N#Cc1cc(-c2ccn3nc(NC(=O)C4CC4)cc3c2)c(OCc2cccnc2)cn1]

The title compound (27 mg, 26%) was prepared as described in Example 1 where pyridin-3-ylmethanol was used instead of (R)-(1-methylpyrrolidin-3-yl)methanol. MS (ESI): mass calcd. for C₂₃H₁₈N₆O₂, 410.1. m/z found, 411.0 [M+H]⁺. ¹H NMR (400 MHz, DMSO-d₆) δ 11.14 (s, 1H), 8.81 (s, 1H), 8.69 (d, J=1.2 Hz, 1H), 8.60 (d, J=7.2 Hz, 1H), 8.57-8.54 (m, 1H), 8.22 (s, 1H), 7.93 (d, J=0.8 Hz, 1H), 7.91-7.87 (m, 1H), 7.43 (dd, J=8.0, 4.8 Hz, 1H), 7.11 (dd, J=7.2, 2.0 Hz, 1H), 6.89 (s, 1H), 5.50 (s, 2H), 1.97-1.88 (m, 1H), 0.86-0.79 (m, 4H).

### Example 48: N-(5-(2-Cyano-5-(pyridin-2-ylmethoxy)pyridin-4-yl)pyrazolo[1,5-a]pyridin-2-yl)cyclopropanecarboxamide

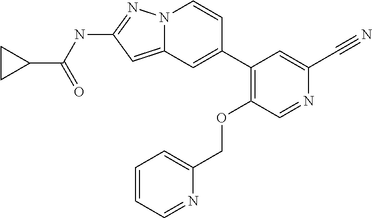 [SMILES: N#Cc1cc(-c2ccn3nc(NC(=O)C4CC4)cc3c2)c(OCc2ccccn2)cn1]

The title compound (16 mg, 16%) was prepared as described in Example 1 where pyridin-2-ylmethanol was used instead of (R)-(1-methylpyrrolidin-3-yl)methanol. MS (ESI): mass calcd. for C₂₃H₁₈N₆O₂, 410.1. m/z found, 411.0 [M+H]⁺; ¹H NMR (400 MHz, DMSO-d₆) δ 11.14 (s, 1H), 8.77 (s, 1H), 8.63 (d, J=7.2 Hz, 1H), 8.60 (d, J=4.4 Hz, 1H), 8.22 (s, 1H), 8.00 (s, 1H), 7.90-7.80 (m, 1H), 7.52 (d, J=7.6 Hz, 1H), 7.40-7.31 (m, 1H), 7.19 (dd, J=6.8, 0.8 Hz, 1H), 6.91 (s, 1H), 5.55 (s, 2H), 1.98-1.86 (m, 1H), 0.89-0.77 (m, 4H).

### Example 49: (*R)—N-(5-(2-Cyano-5-(3,3,3-trifluoro-2-hydroxy-2-methylpropoxy)pyridin-4-yl)pyrazolo[1,5-a]pyridin-2-yl)cyclopropanecarboxamide

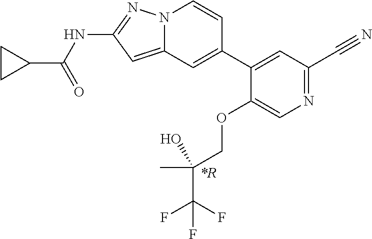 [SMILES: C[C@@](O)(COc1cnc(C#N)cc1-c1ccn2nc(NC(=O)C3CC3)cc2c1)C(F)(F)F]

The title compound (24 mg, 27%) was prepared as described in Example 1 where 3,3,3-trifluoro-2-methylpropane-1,2-diol was used instead of (R)-(1-methylpyrrolidin-3-yl)methanol. The racemic mixture was separated by SFC over DAICEL CHIRALCEL OD-H 250 mm×30 mm, 5 μm (eluent: 40% to 40% (v/v) 0.1% NH₃—H₂O in ETOH). The first eluting isomer was designated as (*R). MS (ESI): mass calcd. for C₂₁H₁₈F₃N₅O₃, 445.1. m/z found, 446.1 [M+H]⁺. ¹H NMR (400 MHz, DMSO-d₆) δ 11.15 (s, 1H), 8.74 (s, 1H), 8.63 (d, J=7.2 Hz, 1H), 8.22 (s, 1H), 7.98 (d, J=0.8 Hz, 1H), 7.13 (dd, J=7.2, 2.0 Hz, 1H), 6.91 (s, 1H), 6.49 (s, 1H), 4.45 (d, J=10.0 Hz, 1H), 4.36 (d, J=10.4 Hz, 1H), 1.98-1.89 (m, 1H), 1.37 (s, 3H), 0.87-0.78 (m, 4H) ¹⁹F NMR (376 MHz, DMSO-d₆) δ −79.61 (s, 3F).

### Example 50: (*S)—N-(5-(2-Cyano-5-(3,3,3-trifluoro-2-hydroxy-2-methylpropoxy)pyridin-4-yl)pyrazolo[1,5-a]pyridin-2-yl)cyclopropanecarboxamide

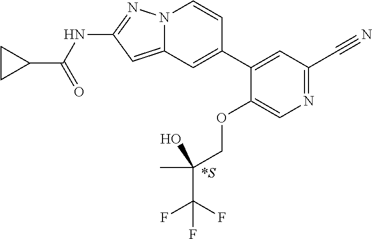 [SMILES: C[C@](O)(COc1cnc(C#N)cc1-c1ccn2nc(NC(=O)C3CC3)cc2c1)C(F)(F)F]

The title compound (13 mg, 15%) was prepared as described in Example 1 where 3,3,3-trifluoro-2-methylpropane-1,2-diol was used instead of (R)-(1-methylpyrrolidin-3-yl)methanol. The racemic mixture was separated by SFC over DAICEL CHIRALCEL OD-H 250 mm×30 mm, 5 μm (eluent: 40% to 40% (v/v) 0.1% NH₃ in H₂O:EtOH). The second eluting isomer was designated as (*S). MS (ESI): mass calcd. for C₂₁H₁₈F₃N₅O₃, 445.1. m/z found, 446.2 [M+H]⁺; ¹H NMR (400 MHz, DMSO-d₆) δ 11.15 (s, 1H), 8.74 (s, 1H), 8.62 (d, J=7.2 Hz, 1H), 8.22 (s, 1H), 7.98 (d, J=0.8 Hz, 1H), 7.13 (dd, J=7.2, 1.6 Hz, 1H), 6.91 (s, 1H), 6.49 (br s, 1H), 4.45 (d, J=10.4 Hz, 1H), 4.36 (d, J=10.4 Hz, 1H), 1.98-1.89 (m, 1H), 1.37 (s, 3H), 0.85-0.80 (m, 4H); ¹⁹F NMR (376 MHz, DMSO-d₆) δ −79.62 (s, 3F).

### Example 51: N-[5-[2-cyano-5-[(1-methyl-4-piperidyl)methoxy]-4-pyridyl]pyrazolo[1,5-a]pyridin-2-yl]cyclopropanecarboxamide

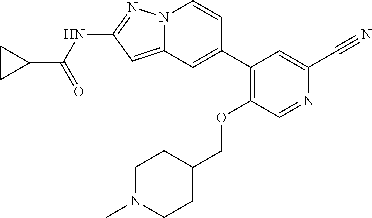 [SMILES: CN1CCC(COc2cnc(C#N)cc2-c2ccn3nc(NC(=O)C4CC4)cc3c2)CC1]

The title compound (28 mg, 33%) was prepared as described in Example 1 where (1-methylpiperidin-4-yl)methanol was used instead of (R)-(1-methylpyrrolidin-3-yl)methanol. MS (ESI): mass calcd. for C₂₄H₂₆N₆O₂, 430.2. m/z found, 431.2 [M+H]⁺; ¹H NMR (400 MHz, DMSO-d₆) δ 11.15 (s, 1H), 8.66 (s, 1H), 8.63 (d, J=7.2 Hz, 1H), 8.17 (s, 1H), 7.90 (s, 1H), 7.08 (dd, J=7.6, 2.0 Hz, 1H), 6.91 (s, 1H), 4.17 (d, J=6.0 Hz, 2H), 2.81-2.73 (m, 2H), 2.14 (s, 3H), 1.97-1.90 (m, 1H), 1.90-1.81 (m, 2H), 1.77-1.62 (m, 3H), 1.35-1.23 (m, 2H), 0.87-0.79 (m, 4H).

### Example 52: N-[5-[2-Cyano-5-(2-hydroxy-2-methyl-propoxy)-4-pyridyl]pyrazolo[1,5-a]pyridin-2-yl]cyclopropanecarboxamide

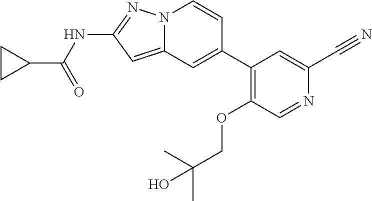 [SMILES: CC(C)(O)COc1cnc(C#N)cc1-c1ccn2nc(NC(=O)C3CC3)cc2c1]

The title compound (35 mg, 46%) was prepared as described in Example 1 where 2-methylpropane-1,2-diol was used instead of (R)-(1-methylpyrrolidin-3-yl)methanol. MS (ESI): mass calcd. for C₂₁H₂₁N₅O₃, 391.2. m/z found, 392.0 [M+H]⁺. ¹H NMR (400 MHz, DMSO-d₆) δ 11.15 (s, 1H), 8.67 (s, 1H), 8.63 (d, J=7.2 Hz, 1H), 8.20 (s, 1H), 8.03 (d, J=1.2 Hz, 1H), 7.17 (dd, J=7.6, 2.0 Hz, 1H), 6.91 (s, 1H), 4.77 (s, 1H), 4.08 (s, 2H), 1.98-1.90 (m, 1H), 1.17 (s, 6H), 0.85-0.80 (m, 4H).

### Example 53: trans-N-(5-(2-Cyano-5-(((1r,4r)-4-hydroxycyclohexyl)methoxy)pyridin-4-yl)pyrazolo[1,5-a]pyridin-2-yl)cyclopropanecarboxamide

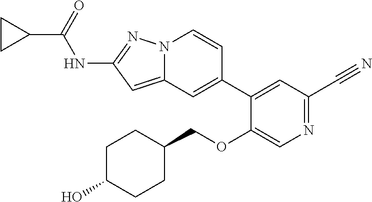 [SMILES: N#Cc1cc(-c2ccn3nc(NC(=O)C4CC4)cc3c2)c(OC[C@H]2CC[C@H](O)CC2)cn1]

The title compound (51 mg, 26%) was prepared as described in Example 1 where (trans)-4-(hydroxymethyl)cyclohexanol was used instead of (R)-(1-methylpyrrolidin-3-yl)methanol. MS (ESI): mass calcd. for C₂₄H₂₅N₅O₃, 431.2. m/z found, 432.2 [M+H]⁺. ¹H NMR (400 MHz, DMSO-d₆) δ 11.13 (s, 1H), 8.69-8.58 (m, 2H), 8.17 (s, 1H), 7.91 (s, 1H), 7.08 (dd, J=1.6, 7.2 Hz, 1H), 6.91 (s, 1H), 4.52 (d, J=4.0 Hz, 1H), 4.13 (d, J=6.0 Hz, 2H), 1.98-1.90 (m, 1H), 1.88-1.79 (m, 2H), 1.78-1.66 (m, 3H), 1.18-1.03 (m, 4H), 0.89-0.77 (m, 4H).

### Example 54: N-[5-[2-Cyano-5-(1-methylazetidin-3-yl)oxy-4-pyridyl]pyrazolo[1,5-a]pyridin-2-yl]cyclopropanecarboxamide

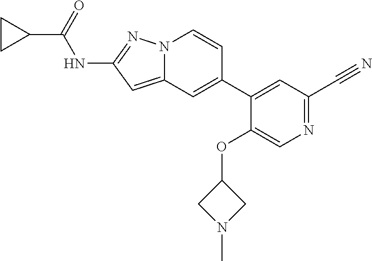 [SMILES: CN1CC(Oc2cnc(C#N)cc2-c2ccn3nc(NC(=O)C4CC4)cc3c2)C1]

The title compound (45 mg, 26%) was prepared as described in Example 1 where 3-hydroxy-1-methylazetidine was used instead of (R)-(1-methylpyrrolidin-3-yl)methanol. MS (ESI): mass calcd. for C₂₁H₂₀N₆O₂, 388.2. m/z found, 389.2 [M+H]⁺. ¹H NMR (400 MHz, DMSO-d₆) δ 11.15 (s, 1H), 8.63 (d, J=7.6 Hz, 1H), 8.38 (s, 1H), 8.21 (s, 1H), 7.95 (s, 1H), 7.13 (d, J=6.8 Hz, 1H), 6.95 (s, 1H), 5.16-5.06 (m, 1H), 3.80-3.68 (m, 2H), 3.13-2.97 (m, 2H), 2.28 (s, 3H), 1.98-1.89 (m, 1H), 0.89-0.79 (m, 4H).

### Example 55: tert-butyl 2-[[6-Cyano-4-[2-(cyclopropanecarbonylamino)pyrazolo[1,5-a]pyridin-5-yl]-3-pyridyl]oxy]acetate

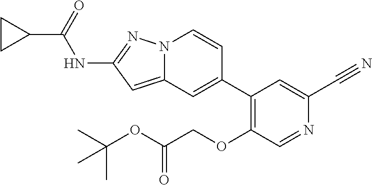 [SMILES: CC(C)(C)OC(=O)COc1cnc(C#N)cc1-c1ccn2nc(NC(=O)C3CC3)cc2c1]

The title compound (12 mg, 2%) was prepared as described in Example 1 where tert-butyl 2-hydroxyacetate was used instead of (R)-(1-methylpyrrolidin-3-yl)methanol. MS (ESI): mass calcd. for C₂₃H₂₃N₅O₄, 433.2. m/z found, 434.2 [M+H]⁺. ¹H NMR (400 MHz, DMSO-d₆) δ 11.13 (s, 1H), 8.64 (d, J=7.2 Hz, 1H), 8.59 (s, 1H), 8.23 (s, 1H), 8.00 (d, J=0.8 Hz, 1H), 7.16 (dd, J=7.2, 2.0 Hz, 1H), 6.92 (s, 1H), 5.07 (s, 2H), 1.98-1.88 (m, 1H), 1.43 (s, 9H), 0.85-0.81 (m, 4H).

### Example 56: (*R)—N-(5-(5-((1-Acetylpyrrolidin-3-yl)oxy)-2-cyanopyridin-4-yl)pyrazolo[1,5-a]pyridin-2-yl)cyclopropanecarboxamide

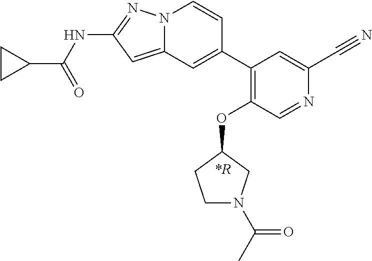 [SMILES: CC(=O)N1CC[C@@H](Oc2cnc(C#N)cc2-c2ccn3nc(NC(=O)C4CC4)cc3c2)C1]

The title compound (41 mg, 10%) was prepared as described in Example 1 where 1-(3-hydroxypyrrolidin-1-yl)ethenone was used instead of (R)-(1-methylpyrrolidin-3-yl)methanol. The racemic mixture was separated by SFC over REGIS (s,s) WHELK-O1 250 mm×30 mm, 5 μm (eluent: 40% to 40% (v/v) 0.1% aq. NH₄OH EtOH). MS (ESI): mass calcd. for C₂₃H₂₂N₆O₃, 430.2. m/z found, 431.2 [M+H]⁺. ¹H NMR (400 MHz, DMSO-d₆) δ 11.13 (s, 1H), 8.74 (s, 1H), 8.64-8.54 (m, 1H), 8.25-8.16 (m, 1H), 7.86 (s, 1H), 7.07-6.97 (m, 1H), 6.92 (s, 1H), 5.53-5.38 (m, 1H), 3.85-3.40 (m, 3H), 3.30-3.16 (m, 1H), 2.38-2.21 (m, 1H), 2.21-2.08 (m, 1H), 1.98-1.86 (m, 4H), 0.92-0.76 (m, 4H).

### Example 57: (*S)—N-(5-(5-((1-Acetylpyrrolidin-3-yl)oxy)-2-cyanopyridin-4-yl)pyrazolo[1,5-a]pyridin-2-yl)cyclopropanecarboxamide

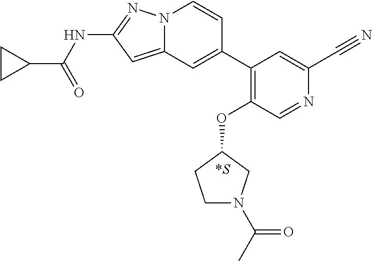 [SMILES: CC(=O)N1CC[C@H](Oc2cnc(C#N)cc2-c2ccn3nc(NC(=O)C4CC4)cc3c2)C1]

The title compound (47 mg, 15%) was prepared as described in Example 1 where 1-(3-hydroxypyrrolidin-1-yl)ethenone was used instead of (R)-(1-methylpyrrolidin-3-yl)methanol. The racemic mixture was separated by SFC over REGIS (s,s) WHELK-O1 250 mm×30 mm, 5 μm (eluent: 40% to 40% (v/v) 0.1% aq. NH₄OH EtOH). MS (ESI): mass calcd. for C₂₃H₂₂N₆O₃, 430.2. m/z found, 431.2 [M+H]⁺. ¹H NMR (400 MHz, DMSO-d₆) δ 11.13 (s, 1H), 8.74 (s, 1H), 8.63-8.54 (m, 1H), 8.23-8.18 (m, 1H), 7.86 (s, 1H), 7.07-6.98 (m, 1H), 6.92 (s, 1H), 5.52-5.39 (m, 1H), 3.86-3.39 (m, 3H), 3.30-3.22 (m, 1H), 2.33-2.21 (m, 1H), 2.20-2.11 (m, 1H), 1.99-1.91 (m, 1H), 1.90 (s, 3H), 0.87-0.79 (m, 4H).

### Example 58: (*R)—N-(5-(2-cyano-5-((1-methylpyrrolidin-3-yl)oxy)pyridin-4-yl)pyrazolo[1,5-a]pyridin-2-yl)cyclopropanecarboxamide

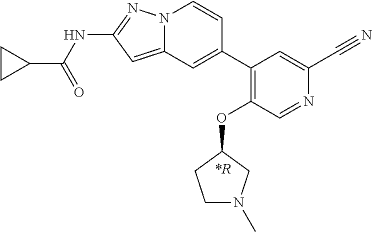 [SMILES: CN1CC[C@@H](Oc2cnc(C#N)cc2-c2ccn3nc(NC(=O)C4CC4)cc3c2)C1]

The title compound (17 mg, 6.7%) was prepared as described in Example 1 where 1-methylpyrrolidin-3-ol was used instead of (R)-(1-methylpyrrolidin-3-yl)methanol. The racemic mixture was separated by SFC over DAICEL CHIRALCEL OJ-H 250 mm×30 mm, 5 μm (eluent: 40% to 40% (v/v) 0.1% aq. NH₄OH-EtOH). MS (ESI): mass calcd. for C₂₂H₂₂N₆O₂, 402.2. m/z found, 403.2 [M+H]⁺. ¹H NMR (400 MHz, DMSO-d₆) δ 11.15 (s, 1H), 8.63-8.58 (m, 2H), 8.19 (s, 1H), 7.95 (s, 1H), 7.13 (dd, J=1.6, 7.2 Hz, 1H), 6.94 (s, 1H), 5.33-5.27 (m, 1H), 2.99-2.90 (m, 1H), 2.89-2.76 (m, 2H), 2.50-2.49 (m, 1H), 2.47-2.39 (m, 1H), 2.34 (s, 3H), 2.00-1.84 (m, 2H), 0.87-0.78 (m, 4H).

### Example 59: (*S)—N-(5-(2-cyano-5-((1-methylpyrrolidin-3-yl)oxy)pyridin-4-yl)pyrazolo[1,5-a]pyridin-2-yl)cyclopropanecarboxamide

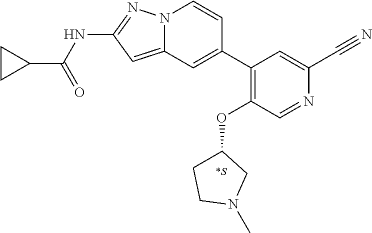 [SMILES: CN1CC[C@H](Oc2cnc(C#N)cc2-c2ccn3nc(NC(=O)C4CC4)cc3c2)C1]

The title compound (26 mg, 10%) was prepared as described in Example 1 where 1-methylpyrrolidin-3-ol was used instead of (R)-(1-methylpyrrolidin-3-yl)methanol. The racemic mixture was separated by SFC over DAICEL CHIRALCEL OJ-H 250 mm×30 mm, 5 μm (eluent: 40% to 40% (v/v) 0.1% aq. NH₄OH-EtOH). MS (ESI): mass calcd. for C₂₂H₂₂N₆O₂, 402.2. m/z found, 403.2 [M+H]⁺. ¹H NMR (400 MHz, DMSO-d₆) δ 11.15 (s, 1H), 8.62 (d, J=7.6 Hz, 1H), 8.59 (s, 1H), 8.19 (s, 1H), 7.96-7.92 (m, 1H), 7.12 (dd, J=2.0, 7.2 Hz, 1H), 6.93 (s, 1H), 5.33-5.23 (m, 1H), 2.93-2.85 (m, 1H), 2.84-2.70 (m, 2H), 2.47-2.35 (m, 2H), 2.30 (s, 3H), 2.00-1.78 (m, 2H), 0.88-0.75 (m, 4H).

### Example 60: N-[5-[5-[(1-acetyl-4-piperidyl)methoxy]-2-cyano-4-pyridyl]pyrazolo[1,5-a]pyridin-2-yl]cyclopropanecarboxamide

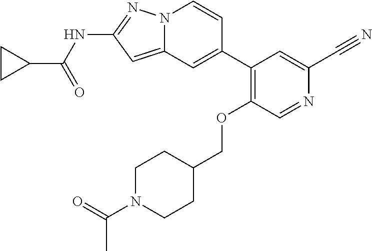 [SMILES: CC(=O)N1CCC(COc2cnc(C#N)cc2-c2ccn3nc(NC(=O)C4CC4)cc3c2)CC1]

The title compound (22 mg, 20%) was prepared as described in Example 1 where 1-(4-(hydroxymethyl)piperidin-1-yl)ethenone was used instead of (R)-(1-methylpyrrolidin-3-yl)methanol. MS (ESI): mass calcd. for C₂₅H₂₆N₆O₃, 458.2. m/z found, 459.2 [M+H]⁺. ¹H NMR (400 MHz, DMSO-d₆) δ 11.16 (s, 1H), 8.67 (s, 1H), 8.62 (d, J=7.2 Hz, 1H), 8.19 (s, 1H), 7.90 (s, 1H), 7.08 (dd, J=2.0, 7.2 Hz, 1H), 6.93 (s, 1H), 4.40-4.31 (m, 1H), 4.24-4.15 (m, 2H), 3.85-3.76 (m, 1H), 3.06-2.96 (m, 1H), 2.58-2.52 (m, 1H), 2.10-2.00 (m, 1H), 1.99-1.90 (m, 4H), 1.78-1.65 (m, 2H), 1.29-1.17 (m, 1H), 1.16-1.04 (m, 1H), 0.89-0.78 (m, 4H).

### Example 61: N-[5-[2-cyano-5-[[6-(trifluoromethyl)-3-pyridyl]methoxy]-4-pyridyl]pyrazolo[1,5-a]pyridin-2-yl]cyclopropanecarboxamide

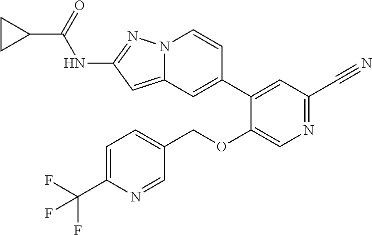 [SMILES: N#Cc1cc(-c2ccn3nc(NC(=O)C4CC4)cc3c2)c(OCc2ccc(C(F)(F)F)nc2)cn1]

The title compound (21 mg, 14%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (6-(trifluoromethyl)pyridin-3-yl)methanol was used instead of (R)-(1-methylpyrrolidin-3-yl)methanol. MS (ESI): mass calcd. for C₂₄H₁₇F₃N₆O₂, 478.1. m/z found, 479.2 [M+H]⁺. ¹H NMR (400 MHz, DMSO-d₆) δ 11.13 (s, 1H), 8.86 (s, 1H), 8.81 (s, 1H), 8.61 (d, J=7.6 Hz, 1H), 8.23 (s, 1H), 8.16 (d, J=8.0 Hz, 1H), 7.98-7.92 (m, 2H), 7.14 (dd, J=2.0, 7.6 Hz, 1H), 6.92 (s, 1H), 5.63 (s, 2H), 1.99-1.88 (m, 1H), 0.87-0.78 (m, 4H). ¹⁹F NMR (376 MHz, DMSO-d₆) δ −66.41 (s, 3F).

### Example 62: N-[5-[2-cyano-5-[[2-(trifluoromethyl)-4-pyridyl]methoxy]-4-pyridyl]pyrazolo[1,5-a]pyridin-2-yl]cyclopropanecarboxamide

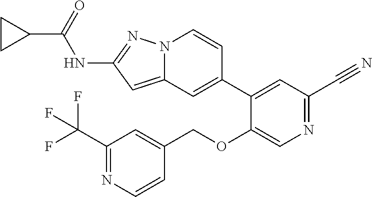 [SMILES: N#Cc1cc(-c2ccn3nc(NC(=O)C4CC4)cc3c2)c(OCc2ccnc(C(F)(F)F)c2)cn1]

The title compound (18 mg, 12%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (2-(trifluoromethyl)pyridin-4-yl)methanol was used instead of (R)-(1-methylpyrrolidin-3-yl)methanol. MS (ESI): mass calcd. for C₂₄H₁₇F₃N₆O₂, 478.1. m/z found, 479.2 [M+H]⁺. ¹H NMR (400 MHz, DMSO-d₆) δ 11.10 (s, 1H), 8.58-8.52 (m, 2H), 8.22 (s, 1H), 7.78 (s, 1H), 7.52 (s, 1H), 7.31 (d, J=4.8 Hz, 1H), 6.75 (s, 1H), 6.72 (d, J=7.2 Hz, 1H), 4.16 (s, 2H), 1.94-1.85 (m, 1H), 0.82-0.76 (m, 4H). ¹⁹F NMR (376 MHz, DMSO-d₆) δ −66.58 (s, 3F).

### Example 63: N-[5-[2-cyano-5-[(2S)-2-hydroxypropoxy]-4-pyridyl]pyrazolo[1,5-a]pyridin-2-yl]cyclopropanecarboxamide

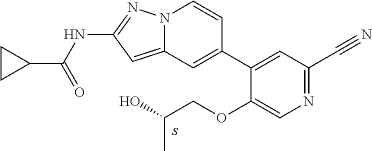

The title compound (20.3 mg, 11%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (S)-propane-1,2-diol was used instead of (R)-(1-methylpyrrolidin-3-yl)methanol. MS (ESI): mass calcd. for C₂₀H₁₉N₅O₃, 377.1. m/z found, 378.1 [M+H]⁺. ¹H NMR (400 MHz, DMSO-d₆) δ 11.15 (s, 1H), 8.68 (s, 1H), 8.61 (d, J=7.2 Hz, 1H), 8.19 (s, 1H), 8.04-7.99 (m, 1H), 7.18 (dd, J=2.0, 7.2 Hz, 1H), 6.92 (s, 1H), 5.03 (d, J=4.8 Hz, 1H), 4.19 (d, J=5.2 Hz, 2H), 4.06-3.95 (m, 1H), 2.03-1.87 (m, 1H), 1.15 (d, J=6.4 Hz, 3H), 0.89-0.77 (m, 4H).

### Example 64: N-[5-[2-cyano-5-(4-hydroxycyclohexoxy)-4-pyridyl]pyrazolo[1,5-a]pyridin-2-yl]cyclopropanecarboxamide

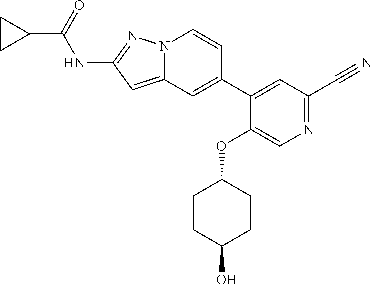 [SMILES: N#Cc1cc(-c2ccn3nc(NC(=O)C4CC4)cc3c2)c(O[C@H]2CC[C@H](O)CC2)cn1]

The title compound (23 mg, 12%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (trans)-cyclohexane-1,4-diol was used instead of (R)-(1-methylpyrrolidin-3-yl)methanol. MS (ESI): mass calcd. for C₂₃H₂₃N₅O₃, 417.2. m/z found, 418.2 [M+H]⁺. ¹H NMR (400 MHz, DMSO-d₆) δ 11.13 (s, 1H), 8.72 (s, 1H), 8.60 (d, J=7.2 Hz, 1H), 8.17 (s, 1H), 7.89 (d, J=0.8 Hz, 1H), 7.08 (dd, J=2.0, 7.6 Hz, 1H), 6.92 (s, 1H), 4.86-4.76 (m, 1H), 4.59 (d, J=3.6 Hz, 1H), 3.61-3.48 (m, 1H), 2.10-2.00 (m, 2H), 1.98-1.89 (m, 1H), 1.80-1.70 (m, 2H), 1.56-1.44 (m, 2H), 1.43-1.31 (m, 2H), 0.98-0.68 (m, 4H).

### Example 65: N-[5-[2-cyano-5-[[(3S)-1-methyl-3-piperidyl]oxy]-4-pyridyl]pyrazolo[1,5-a]pyridin-2-yl]cyclopropanecarboxamide

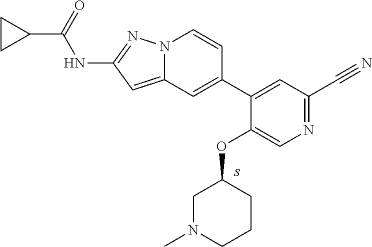

The title compound (31.5 mg, 16%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (S)-1-methylpiperidin-3-ol was used instead of (R)-(1-methylpyrrolidin-3-yl)methanol. MS (ESI): mass calcd. for C₂₃H₂₄N₆O₂, 416.2. m/z found, 417.2 [M+H]⁺. ¹H NMR (400 MHz, DMSO-d₆) δ 11.15 (s, 1H), 8.73 (s, 1H), 8.61 (d, J=7.2 Hz, 1H), 8.19 (s, 1H), 8.04 (d, J=0.8 Hz, 1H), 7.19 (dd, J=2.0, 7.6 Hz, 1H), 6.91 (s, 1H), 4.99-4.84 (m, 1H), 2.76-2.61 (m, 1H), 2.44-2.32 (m, 2H), 2.30-2.20 (m, 1H), 2.17 (s, 3H), 2.04-1.86 (m, 2H), 1.78-1.65 (m, 1H), 1.61-1.42 (m, 2H), 0.89-0.76 (m, 4H).

### Example 66: N-[5-[2-cyano-5-[[(1S,5R)-8-methyl-8-azabicyclo[3.2.1]octan-3-yl]oxy]-4-pyridyl]pyrazolo[1,5-a]pyridin-2-yl]cyclopropanecarboxamide

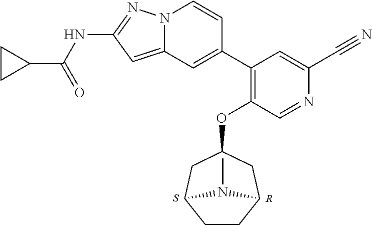

The title compound (49 mg, 23%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (1R,3r,5S)-8-methyl-8-azabicyclo[3.2.1]octan-3-ol was used instead of (R)-(1-methylpyrrolidin-3-yl)methanol. MS (ESI): mass calcd. for C₂₅H₂₆N₆O₂, 442.2. m/z found, 443.2 [M+H]⁺. ¹H NMR (400 MHz, DMSO-d₆) δ 11.14 (s, 1H), 8.63 (d, J=7.2 Hz, 1H), 8.51 (s, 1H), 8.14 (s, 1H), 7.88 (s, 1H), 7.07 (dd, J=2.0, 7.2 Hz, 1H), 6.92 (s, 1H), 5.03-4.92 (m, 1H), 2.95 (s, 2H), 2.16-2.04 (m, 5H), 1.99-1.89 (m, 1H), 1.80 (s, 1H), 1.77 (s, 1H), 1.71-1.61 (m, 2H), 1.50-1.43 (m, 2H), 0.88-0.78 (m, 4H).

### Example 67: N-[5-[5-[[(1S,5R)-8-azabicyclo[3.2.1]octan-3-yl]methoxy]-2-cyano-4-pyridyl]pyrazolo[1,5-a]pyridin-2-yl]cyclopropanecarboxamide

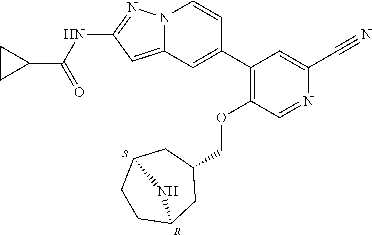

The title compound (6.9 mg, 3.3%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (1R,3s,5S)-8-azabicyclo[3.2.1]octan-3-ylmethanol was used instead of (R)-(1-methylpyrrolidin-3-yl)methanol. MS (ESI): mass calcd. for C₂₅H₂₆N₆O₂, 442.2. m/z found, 443.2 [M+H]⁺. ¹H NMR (400 MHz, DMSO-d₆) δ 11.15 (s, 1H), 8.69-8.60 (m, 2H), 8.18 (s, 1H), 7.93 (s, 1H), 7.10 (d, J=6.4 Hz, 1H), 6.92 (s, 1H), 4.07 (d, J=6.4 Hz, 2H), 3.45-3.39 (m, 2H), 2.24-2.09 (m, 1H), 2.02-1.86 (m, 1H), 1.73-1.61 (m, 2H), 1.59-1.43 (m, 4H), 1.38-1.25 (m, 2H), 0.91-0.76 (m, 4H).

### Example 68: N-[5-[2-cyano-5-[(3-hydroxycyclobutyl)methoxy]-4-pyridyl]pyrazolo[1,5-a]pyridin-2-yl]cyclopropanecarboxamide

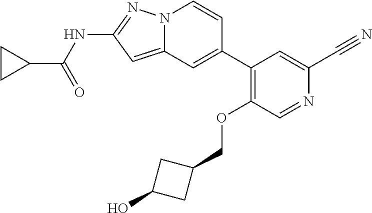 [SMILES: N#Cc1cc(-c2ccn3nc(NC(=O)C4CC4)cc3c2)c(OC[C@H]2C[C@@H](O)C2)cn1]

The title compound (16 mg, 47%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and 3-(hydroxymethyl)cyclobutanol was used instead of (R)-(1-methylpyrrolidin-3-yl)methanol. MS (ESI): mass calcd. for C₂₂H₂₁N₅O₃, 403.2. m/z found, 404.2 [M+H]⁺. ¹H NMR (400 MHz, DMSO-d₆) δ 11.14 (s, 1H), 8.65 (s, 1H), 8.61 (d, J=7.2 Hz, 1H), 8.19 (s, 1H), 7.94 (d, J=1.2 Hz, 1H), 7.11 (dd, J=2.0, 7.2 Hz, 1H), 6.93 (s, 1H), 5.07 (s, 1H), 4.27 (d, J=6.4 Hz, 2H), 4.05-3.95 (m, 1H), 2.34-2.24 (m, 2H), 2.22-2.08 (m, 1H), 1.99-1.88 (m, 1H), 1.74-1.61 (m, 2H), 0.87-0.79 (m, 4H).

### Example 69: N-[5-[2-cyano-5-[3-(hydroxymethyl)cyclobutoxy]-4-pyridyl]pyrazolo[1,5-a]pyridin-2-yl]cyclopropanecarboxamide

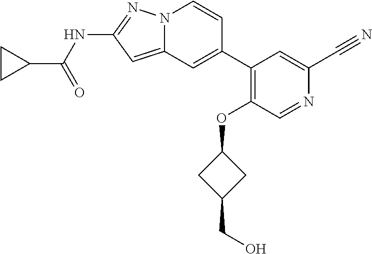 [SMILES: N#Cc1cc(-c2ccn3nc(NC(=O)C4CC4)cc3c2)c(O[C@H]2C[C@@H](CO)C2)cn1]

The title compound (22 mg, 63%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and 3-(hydroxymethyl)cyclobutanol was used instead of (R)-(1-methylpyrrolidin-3-yl)methanol. MS (ESI): mass calcd. for C₂₂H₂₁N₅O₃, 403.2. m/z found, 404.2 [M+H]⁺. ¹H NMR (400 MHz, DMSO-d₆) δ 11.14 (s, 1H), 8.62 (d, J=7.6 Hz, 1H), 8.47 (s, 1H), 8.19 (s, 1H), 7.94 (d, J=1.2 Hz, 1H), 7.12 (dd, J=1.6, 7.2 Hz, 1H), 6.94 (s, 1H), 5.00-4.90 (m, 1H), 4.60 (t, J=5.6 Hz, 1H), 3.39 (t, J=5.2 Hz, 2H), 2.59-2.52 (m, 2H), 2.15-2.02 (m, 1H), 1.98-1.83 (m, 3H), 0.88-0.78 (m, 4H).

### Example 70: N-[5-[2-cyano-5-(3-hydroxyazetidin-1-yl)-4-pyridyl]pyrazolo[1,5-a]pyridin-2-yl]cyclopropanecarboxamide

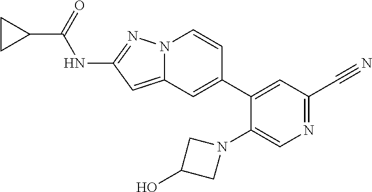 [SMILES: N#Cc1cc(-c2ccn3nc(NC(=O)C4CC4)cc3c2)c(N2CC(O)C2)cn1]

The title compound (61 mg, 40%) was prepared as described in Example 2 where azetidin-3-ol was used instead of N,N-dimethylethylenediamine. MS (ESI): mass calcd. for C₂₀H₁₈N₆O₂, 374.1. m/z found, 375.2 [M+H]⁺. ¹H NMR (400 MHz, DMSO-d₆) δ 11.09 (s, 1H), 8.58 (dt, J=7.1, 0.9 Hz, 1H), 8.01 (s, 1H), 7.72 (s, 1H), 7.57 (dd, J=2.0, 0.9 Hz, 1H), 6.89 (s, 1H), 6.82 (dd, J=7.1, 2.0 Hz, 1H), 5.59 (d, J=6.0 Hz, 1H), 4.46-4.35 (m, 1H), 4.01-3.92 (m, 2H), 3.57-3.49 (m, 2H), 2.00-1.86 (m, 1H), 0.88-0.76 (m, 4H).

### Example 71: N-[5-[2-cyano-5-[[(3R)-1-methylpyrrolidin-3-yl]methylamino]-4-pyridyl]pyrazolo[1,5-a]pyridin-2-yl]cyclopropanecarboxamide

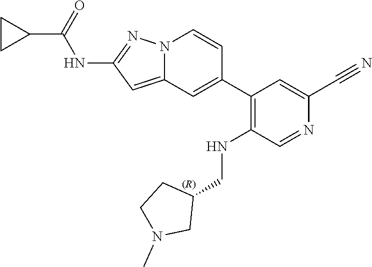

The title compound (96 mg, 56%) was prepared as described in Example 2 where (R)-1-methyl-3-(aminomethyl)pyrrolidine was used instead of N,N-dimethylethylenediamine. MS (ESI): mass calcd. for C₂₃H₂₅N₇O, 415.2. m/z found, 416.2 [M+H]⁺. ¹H NMR (400 MHz, DMSO-d₆) δ 11.08 (s, 1H), 8.64 (d, J=7.1 Hz, 1H), 8.12 (s, 1H), 7.68-7.57 (m, 2H), 6.89 (s, 1H), 6.79 (dd, J=7.1, 2.0 Hz, 1H), 6.68 (t, J=4.9 Hz, 1H), 3.24-3.09 (m, 2H), 2.59 (td, J=8.5, 3.7 Hz, 1H), 2.39 (dd, J=9.2, 3.1 Hz, 1H), 2.24 (dd, J=9.2, 7.1 Hz, 1H), 2.08 (q, J=8.3 Hz, 1H), 1.97-1.86 (m, 5H), 1.58-1.42 (m, 1H), 0.89-0.72 (m, 4H).

### Example 72: N-[5-[2-cyano-5-[3-(dimethylaminomethyl)azetidin-1-yl]-4-pyridyl]pyrazolo[1,5-a]pyridin-2-yl]cyclopropanecarboxamide

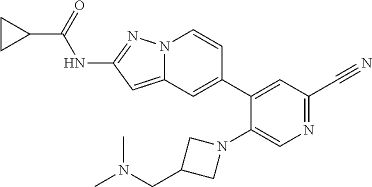 [SMILES: CN(C)CC1CN(c2cnc(C#N)cc2-c2ccn3nc(NC(=O)C4CC4)cc3c2)C1]

The title compound (65 mg, 48%) was prepared as described in Example 2 where 1-(azetidin-3-yl)-N,N-dimethylmethanamine dihydrochloride was used instead of N,N-dimethylethylenediamine. MS (ESI): mass calcd. for C₂₃H₂₅N₇O, 415.2. m/z found, 416.2 [M+H]⁺. ¹H NMR (400 MHz, DMSO-d₆) δ 11.09 (s, 1H), 8.57 (dd, J=7.1, 1.0 Hz, 1H), 8.00 (s, 1H), 7.70 (s, 1H), 7.60-7.54 (m, 1H), 6.89 (s, 1H), 6.83 (dd, J=7.1, 2.0 Hz, 1H), 3.86 (t, J=8.3 Hz, 2H), 3.43 (dd, J=8.5, 5.7 Hz, 2H), 2.83-2.68 (m, 1H), 2.35 (d, J=7.6 Hz, 2H), 2.03 (s, 6H), 1.98-1.88 (m, 1H), 0.87-0.78 (m, 4H).

### Example 73: 1-[6-cyano-4-[2-(cyclopropanecarbonylamino)pyrazolo[1,5-a]pyridin-5-yl]-3-pyridyl]-N-methyl-azetidine-3-carboxamide

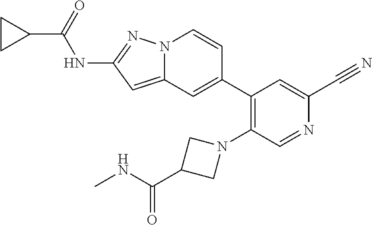 [SMILES: CNC(=O)C1CN(c2cnc(C#N)cc2-c2ccn3nc(NC(=O)C4CC4)cc3c2)C1]

The title compound (35 mg, 19%) was prepared as described in Example 3 where N-methylazetidine-3-carboxamide was used instead of azetidin-3-ylmethanol. MS (ESI): mass calcd. for C₂₂H₂₁N₇O₂, 415.2. m/z found, 416.2 [M+H]⁺. ¹H NMR (400 MHz, DMSO-d₆) δ 11.11 (s, 1H), 8.59 (d, J=7.2 Hz, 1H), 8.04 (s, 1H), 7.85-7.79 (m, 1H), 7.74 (s, 1H), 7.59 (d, J=0.8 Hz, 1H), 6.89 (s, 1H), 6.84 (dd, J=2.0, 7.2 Hz, 1H), 3.96-3.86 (m, 2H), 3.83-3.71 (m, 2H), 3.32-3.27 (m, 1H), 2.52 (d, J=4.4 Hz, 3H), 1.97-1.88 (m, 1H), 0.89-0.76 (m, 4H).

### Example 74: N-[5-[2-cyano-5-[3-(1-hydroxy-1-methyl-ethyl)azetidin-1-yl]-4-pyridyl]pyrazolo[1,5-a]pyridin-2-yl]cyclopropanecarboxamide

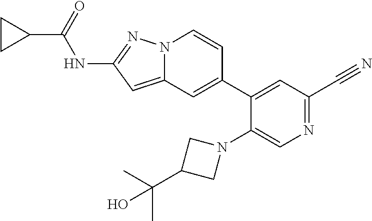 [SMILES: CC(C)(O)C1CN(c2cnc(C#N)cc2-c2ccn3nc(NC(=O)C4CC4)cc3c2)C1]

The title compound (13.5 mg, 7%) was prepared as described in Example 3 where 2-(azetidin-3-yl)propan-2-ol hydrochloride was used instead of azetidin-3-ylmethanol. MS (ESI): mass calcd. for C₂₃H₂₄N₆O₂, 416.2. m/z found, 417.2 [M+H]⁺. ¹H NMR (400 MHz, DMSO-d₆) δ 11.12 (s, 1H), 8.58 (d, J=7.2 Hz, 1H), 8.02 (s, 1H), 7.71 (s, 1H), 7.59 (d, J=0.8 Hz, 1H), 6.90 (s, 1H), 6.84 (dd, J=2.0, 7.2 Hz, 1H), 4.43 (s, 1H), 3.80-3.71 (m, 2H), 3.70-3.60 (m, 2H), 2.64-2.54 (m, 1H), 1.98-1.87 (m, 1H), 0.94 (s, 6H), 0.87-0.77 (m, 4H).

### Example 75: N-[5-[2-cyano-5-[3-hydroxy-3-(trifluoromethyl)azetidin-1-yl]-4-pyridyl]pyrazolo[1,5-a]pyridin-2-yl]cyclopropanecarboxamide

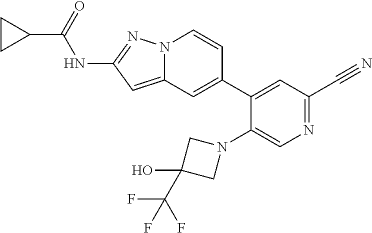 [SMILES: N#Cc1cc(-c2ccn3nc(NC(=O)C4CC4)cc3c2)c(N2CC(O)(C(F)(F)F)C2)cn1]

The title compound (25 mg, 13%) was prepared as described in Example 3 where 3-(trifluoromethyl)azetidin-3-ol hydrochloride was used instead of azetidin-3-ylmethanol. MS (ESI): mass calcd. for C₂₁H₁₇F₃N₆O₂, 442.1. m/z found, 443.1 [M+H]⁺. ¹H NMR (400 MHz, DMSO-d₆) δ 11.14 (s, 1H), 8.63 (d, J=7.2 Hz, 1H), 8.17 (s, 1H), 7.82 (s, 1H), 7.65-7.59 (m, 1H), 7.30 (s, 1H), 6.91 (s, 1H), 6.84 (dd, J=2.0, 7.2 Hz, 1H), 4.03 (d, J=10.4 Hz, 2H), 3.81 (d, J=9.6 Hz, 2H), 1.98-1.88 (m, 1H), 0.88-0.77 (m, 4H). ¹⁹F NMR (376 MHz, DMSO-d₆) δ −82.65 (s, 3F).

### Example 76: N-[5-[2-cyano-5-(cyclopropylmethylamino)-4-pyridyl]pyrazolo[1,5-a]pyridin-2-yl]cyclopropanecarboxamide

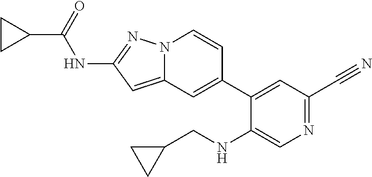 [SMILES: N#Cc1cc(-c2ccn3nc(NC(=O)C4CC4)cc3c2)c(NCC2CC2)cn1]

The title compound (28 mg, 24%) was prepared as described in Example 3 where cyclopropylmethanamine was used instead of azetidin-3-ylmethanol. MS (ESI): mass calcd. for C₂₁H₂₀N₆O, 372.2. m/z found, 373.2 [M+H]⁺. ¹H NMR (400 MHz, DMSO-d₆) δ 11.11 (s, 1H), 8.63 (d, J=7.2 Hz, 1H), 8.25 (s, 1H), 7.66 (d, J=1.2 Hz, 1H), 7.64 (s, 1H), 6.90 (s, 1H), 6.81 (dd, J=2.0, 7.2 Hz, 1H), 6.26 (t, J=6.0 Hz, 1H), 3.14 (t, J=6.4 Hz, 2H), 1.97-1.88 (m, 1H), 1.16-1.02 (m, 1H), 0.88-0.78 (m, 4H), 0.49-0.40 (m, 2H), 0.26-0.19 (m, 2H).

### Example 77: N-[5-[2-cyano-5-[(1-methylpyrazol-3-yl)amino]-4-pyridyl]pyrazolo[1,5-a]pyridin-2-yl]cyclopropanecarboxamide

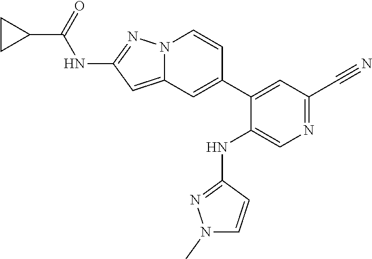 [SMILES: Cn1ccc(Nc2cnc(C#N)cc2-c2ccn3nc(NC(=O)C4CC4)cc3c2)n1]

The title compound (31 mg, 25%) was prepared as described in Example 3 where 1-methyl-1H-pyrazol-3-amine was used instead of azetidin-3-ylmethanol. MS (ESI): mass calcd. for C₂₁H₁₈N₈O, 398.2. m/z found, 399.2 [M+H]⁺. ¹H NMR (400 MHz, DMSO-d₆) δ 11.11 (s, 1H), 9.32 (s, 1H), 8.61 (d, J=7.2 Hz, 1H), 8.39 (s, 1H), 7.79 (s, 1H), 7.72 (s, 1H), 7.57 (d, J=2.0 Hz, 1H), 6.91 (s, 1H), 6.84 (dd, J=2.0, 7.2 Hz, 1H), 5.91 (d, J=2.0 Hz, 1H), 3.78 (s, 3H), 1.98-1.88 (m, 1H), 0.88-0.78 (m, 4H).

### Example 78: N-[5-[2-cyano-5-(cyclopentylamino)-4-pyridyl]pyrazolo[1,5-a]pyridin-2-yl]cyclopropanecarboxamide

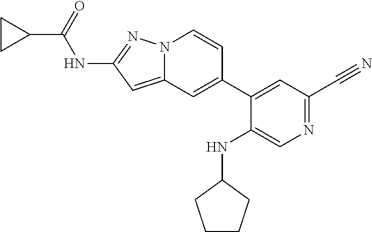 [SMILES: N#Cc1cc(-c2ccn3nc(NC(=O)C4CC4)cc3c2)c(NC2CCCC2)cn1]

The title compound (26 mg, 21%) was prepared as described in Example 3 where cyclopentanamine was used instead of azetidin-3-ylmethanol. MS (ESI): mass calcd. for C₂₂H₂₂N₆O, 386.2. m/z found, 387.2 [M+H]⁺. ¹H NMR (400 MHz, DMSO-d₆) δ 11.10 (s, 1H), 8.59 (d, J=7.2 Hz, 1H), 8.22 (s, 1H), 7.66 (d, J=0.8 Hz, 1H), 7.64 (s, 1H), 6.89 (s, 1H), 6.81 (dd, J=2.0, 7.2 Hz, 1H), 5.87 (d, J=6.8 Hz, 1H), 4.05-3.94 (m, 1H), 2.02-1.88 (m, 3H), 1.67-1.44 (m, 6H), 0.88-0.77 (m, 4H).

### Example 79: N-[5-[2-cyano-5-[(1-methylpyrazol-4-yl)amino]-4-pyridyl]pyrazolo[1,5-a]pyridin-2-yl]cyclopropanecarboxamide

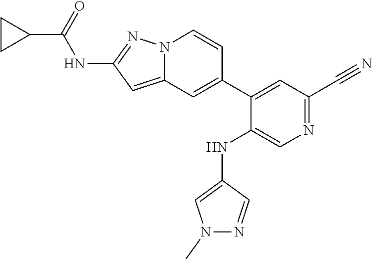 [SMILES: Cn1cc(Nc2cnc(C#N)cc2-c2ccn3nc(NC(=O)C4CC4)cc3c2)cn1]

The title compound (33 mg, 26%) was prepared as described in Example 3 where 1-methyl-1H-pyrazol-4-amine was used instead of azetidin-3-ylmethanol. MS (ESI): mass calcd. for C₂₁H₁₈N80, 398.2. m/z found, 399.2 [M+H]⁺. ¹H NMR (400 MHz, DMSO-d₆) δ11.09 (s, 1H), 8.61 (d, J=7.2 Hz, 1H), 8.23 (s, 1H), 7.80 (s, 1H), 7.75 (s, 1H), 7.74 (s, 1H), 7.72 (d, J=1.2 Hz, 1H), 7.40 (s, 1H), 6.90 (s, 1H), 6.87 (dd, J=2.0, 7.2 Hz, 1H), 3.81 (s, 3H), 1.99-1.87 (m, 1H), 0.87-0.75 (m, 4H).

### Example 80: N-[5-[2-cyano-5-(cyclopropylamino)-4-pyridyl]pyrazolo[1,5-a]pyridin-2-yl]cyclopropanecarboxamide

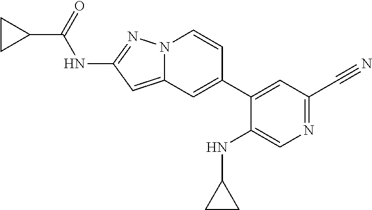 [SMILES: N#Cc1cc(-c2ccn3nc(NC(=O)C4CC4)cc3c2)c(NC2CC2)cn1]

The title compound (30 mg, 26%) was prepared as described in Example 3 where cyclopropanamine was used instead of azetidin-3-ylmethanol. MS (ESI): mass calcd. for C₂₀H₁₈N₆O, 358.2. m/z found, 359.2 [M+H]⁺. ¹H NMR (400 MHz, DMSO-d₆) δ 11.11 (s, 1H), 8.58 (d, J=7.2 Hz, 1H), 8.48 (s, 1H), 7.66 (s, 1H), 7.64-7.61 (m, 1H), 6.88 (s, 1H), 6.78 (dd, J=2.0, 7.2 Hz, 1H), 6.61 (s, 1H), 2.54-2.51 (m, 1H), 1.98-1.85 (m, 1H), 0.88-0.72 (m, 6H), 0.56-0.48 (m, 2H).

### Example 81: N-[5-[2-cyano-5-[(2-hydroxy-2-methyl-propyl)amino]-4-pyridyl]pyrazolo[1,5-a]pyridin-2-yl]cyclopropanecarboxamide

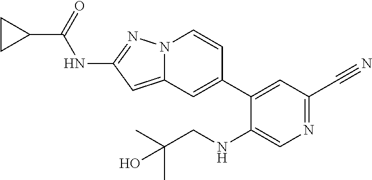 [SMILES: CC(C)(O)CNc1cnc(C#N)cc1-c1ccn2nc(NC(=O)C3CC3)cc2c1]

The title compound (22 mg, 63%) was prepared as described in Example 3 where 1-amino-2-methylpropan-2-ol was used instead of azetidin-3-ylmethanol. MS (ESI): mass calcd. for C₂₁H₂₂N₆O₂, 390.2. m/z found, 391.2 [M+H]⁺. ¹H NMR (400 MHz, DMSO-d₆) δ 11.12 (s, 1H), 8.66 (d, J=7.2 Hz, 1H), 8.32 (s, 1H), 7.73-7.70 (m, 1H), 7.66 (s, 1H), 6.90 (s, 1H), 6.87 (dd, J=1.6, 7.2 Hz, 1H), 5.77 (t, J=6.0 Hz, 1H), 4.64 (s, 1H), 3.18 (d, J=6.0 Hz, 2H), 1.98-1.88 (m, 1H), 1.13 (s, 6H), 0.87-0.77 (m, 4H).

### Example 82: (3-endo)-N-(5-(5-(((1R,3r,5S)-8-azabicyclo[3.2.1]octan-3-yl)oxy)-2-cyanopyridin-4-yl)pyrazolo[1,5-a]pyridin-2-yl)cyclopropanecarboxamide

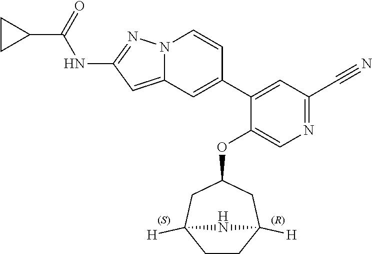

The title compound (32 mg, 23%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (1R,3r,5S)-8-azabicyclo[3.2.1]octan-3-ol was used instead of (R)-(1-methylpyrrolidin-3-yl)methanol. MS (ESI): mass calcd. for C₂₄H₂₄N₆O₂, 428.2. m/z found, 429.2 [M+H]⁺. ¹H NMR (400 MHz, DMSO-d₆) δ 11.14 (s, 1H), 8.64 (d, J=7.2 Hz, 1H), 8.51 (s, 1H), 8.14 (s, 1H), 7.88 (d, J=1.2 Hz, 1H), 7.07 (dd, J=2.0, 7.2 Hz, 1H), 6.92 (s, 1H), 5.06-4.99 (m, 1H), 3.29-3.26 (m, 2H), 2.06-1.97 (m, 2H), 1.96-1.90 (m, 1H), 1.86-1.82 (m, 1H), 1.82-1.77 (m, 1H), 1.56-1.49 (m, 2H), 1.43-1.32 (m, 2H), 0.87-0.79 (m, 4H).

### Example 83: N-(5-(2-cyano-5-(((1R,3R,5S)-8-methyl-8-azabicyclo[3.2.1]octan-3-yl)methoxy)pyridin-4-yl)pyrazolo[1,5-a]pyridin-2-yl)cyclopropanecarboxamide

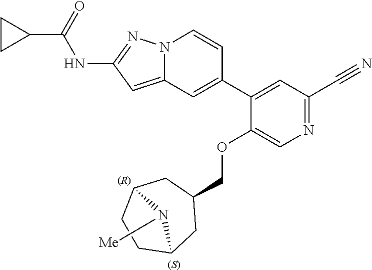

The title compound (118 mg, 33%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and ((1R,3r,5S)-8-methyl-8-azabicyclo[3.2.1]octan-3-yl)methanol was used instead of (R)-(1-methylpyrrolidin-3-yl)methanol. MS (ESI): mass calcd. for C₂₆H₂₈N₆O₂, 456.2. m/z found, 457.2 [M+H]⁺. ¹H NMR (400 MHz, DMSO-d₆) δ 11.13 (s, 1H), 8.66-8.59 (m, 2H), 8.18 (s, 1H), 7.94 (d, J=1.2 Hz, 1H), 7.09 (dd, J=2.0, 7.2 Hz, 1H), 6.92 (s, 1H), 4.10 (d, J=6.4 Hz, 2H), 3.09-2.99 (m, 2H), 2.14-2.04 (m, 4H), 1.98-1.87 (m, 3H), 1.51-1.39 (m, 6H), 0.88-0.78 (m, 4H).

### Example 84: N-[5-[2-cyano-5-[[(6S)-1,4-oxazepan-6-yl]oxy]-4-pyridyl]pyrazolo[1,5-a]pyridin-2-yl]cyclopropanecarboxamide

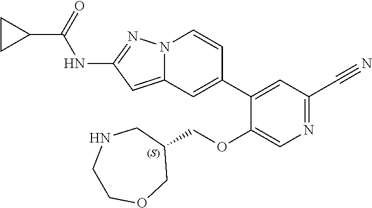

The title compound (62 mg, 31%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (S)-1,4-oxazepan-6-ol was used instead of (R)-(1-methylpyrrolidin-3-yl)methanol. MS (ESI): mass calcd. for C₂₂H₂₂N₆O₃, 418.2. m/z found, 419.2 [M+H]⁺. ¹H NMR (400 MHz, DMSO-d₆) δ 11.14 (s, 1H), 8.64 (s, 1H), 8.62 (d, J=7.2 Hz, 1H), 8.18 (s, 1H), 7.98 (d, J=1.2 Hz, 1H), 7.16 (dd, J=2.0, 7.2 Hz, 1H), 6.92 (s, 1H), 5.11-5.02 (m, 1H), 4.04-3.98 (m, 1H), 3.93-3.86 (m, 1H), 3.74-3.67 (m, 1H), 3.57-3.50 (m, 1H), 3.30-3.23 (m, 1H), 2.92-2.86 (m, 1H), 2.85-2.73 (m, 2H), 1.98-1.89 (m, 1H), 0.86-0.79 (m, 4H).

### Example 85: N-[5-[2-cyano-5-[(1-methyl-4-piperidyl)oxy]-4-pyridyl]pyrazolo[1,5-a]pyridin-2-yl]cyclopropanecarboxamide

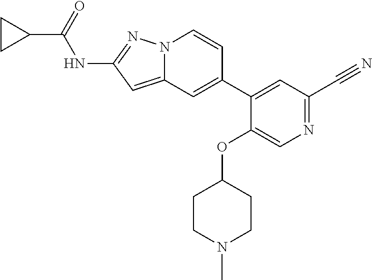 [SMILES: CN1CCC(Oc2cnc(C#N)cc2-c2ccn3nc(NC(=O)C4CC4)cc3c2)CC1]

The title compound (31 mg, 12%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and 1-methylpiperidin-4-ol was used instead of (R)-(1-methylpyrrolidin-3-yl)methanol. MS (ESI): mass calcd. for C₂₃H₂₄N₆O₂, 416.2. m/z found, 417.2 [M+H]⁺. ¹H NMR (400 MHz, DMSO-d₆) δ 11.16 (s, 1H), 8.72 (s, 1H), 8.63 (d, J=7.2 Hz, 1H), 8.18 (s, 1H), 7.93 (s, 1H), 7.10 (d, J=6.8 Hz, 1H), 6.93 (s, 1H), 4.93-4.82 (m, 1H), 2.47-2.34 (s, 2H), 2.32-2.19 (m, 2H), 2.12 (s, 3H), 2.01-1.87 (m, 3H), 1.76-1.67 (m, 2H), 0.88-0.75 (m, 4H).

### Example 86: N-(5-(2-cyano-5-(((1s,4s)-4-hydroxycyclohexyl)oxy)pyridin-4-yl)pyrazolo[1,5-a]pyridin-2-yl)cyclopropanecarboxamide

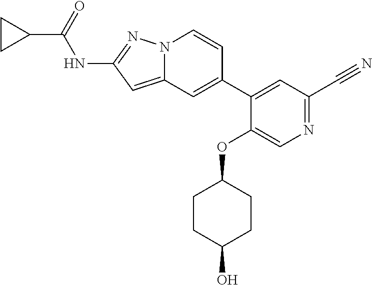 [SMILES: N#Cc1cc(-c2ccn3nc(NC(=O)C4CC4)cc3c2)c(O[C@H]2CC[C@@H](O)CC2)cn1]

The title compound (14 mg, 7.3%) was prepared as described Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (cis)-cyclohexane-1,4-diol was used instead of (R)-(1-methylpyrrolidin-3-yl)methanol. MS (ESI): mass calcd. for C₂₃H₂₃N₅O₃, 417.2. m/z found, 418.2 [M+H]⁺. ¹H NMR (400 MHz, DMSO-d₆) δ 11.14 (s, 1H), 8.69 (s, 1H), 8.62 (d, J=7.2 Hz, 1H), 8.19 (s, 1H), 7.94 (d, J=0.8 Hz, 1H), 7.12 (dd, J=2.0, 7.8 Hz, 1H), 6.93 (s, 1H), 4.90 (m, 1H), 4.53 (d, J=4.4 Hz, 1H), 3.60-3.51 (m, 1H), 1.96-1.85 (m, 3H), 1.73-1.54 (m, 4H), 1.50-1.36 (m, 2H), 0.85-0.80 (m, 4H).

### Example 87: (rac-)-N-[5-[2-cyano-5-[3-hydroxy-3-(trifluoromethyl)pyrrolidin-1-yl]-4-pyridyl]pyrazolo[1,5-a]pyridin-2-yl]cyclopropanecarboxamide

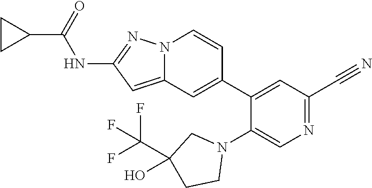 [SMILES: N#Cc1cc(-c2ccn3nc(NC(=O)C4CC4)cc3c2)c(N2CCC(O)(C(F)(F)F)C2)cn1]

The title compound (74 mg, 13%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (RS)-3-(trifluoromethyl)pyrrolidin-3-ol hydrochloride was used instead of (R)-(1-methylpyrrolidin-3-yl)methanol. MS (ESI): mass calcd. for C₂₂H₁₉F₃N₆O₂, 456.2. m/z found, 457.2 [M+H]⁺. ¹H NMR (400 MHz, DMSO-d₆) δ 11.13 (s, 1H), 8.60 (d, J=7.2 Hz, 1H), 8.36 (s, 1H), 7.78 (s, 1H), 7.61 (d, J=1.2 Hz, 1H), 6.89 (s, 1H), 6.85 (dd, J=2.0, 6.8 Hz, 1H), 6.49 (s, 1H), 3.52-3.43 (m, 2H), 3.40-3.36 (m, 1H), 3.12 (d, J=11.6 Hz, 1H), 2.20-2.08 (m, 1H), 2.03-1.89 (m, 2H), 0.86-0.80 (m, 4H). ¹H NMR (400 MHz, CD₃CN) δ 9.17 (s, 1H), 8.35 (d, J=7.2 Hz, 1H), 8.25 (s, 1H), 7.59 (s, 1H), 7.46 (d, J=1.2 Hz, 1H), 6.92 (s, 1H), 6.79 (dd, J=2.0, 7.2 Hz, 1H), 3.56 (d, J=11.2 Hz, 1H), 3.53-3.46 (m, 1H), 3.37-3.28 (m, 1H), 3.14 (d, J=11.2 Hz, 1H), 2.26-2.21 (m, 1H), 2.04-1.96 (m, 1H), 1.84-1.74 (m, 1H), 0.98-0.90 (m, 2H), 0.89-0.80 (m, 2H).

### Example 88: (R)—N-(5-(2-cyano-5-((1-methylpiperidin-3-yl)oxy)pyridin-4-yl)pyrazolo[1,5-a]pyridin-2-yl)cyclopropanecarboxamide

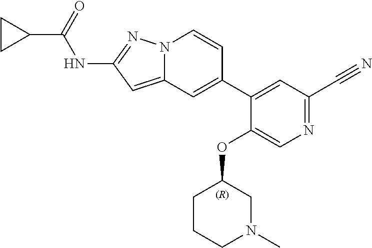

The title compound (29 mg, 15%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (R)-1-methylpiperidin-3-ol was used instead of (R)-(1-methylpyrrolidin-3-yl)methanol. MS (ESI): mass calcd. for C₂₃H₂₄N₆O₂, 416.2. m/z found, 417.2 [M+H]⁺. ¹H NMR (400 MHz, DMSO-d₆) δ 11.15 (s, 1H), 8.73 (s, 1H), 8.61 (d, J=7.2 Hz, 1H), 8.19 (s, 1H), 8.04 (d, J=0.8 Hz, 1H), 7.19 (dd, J=2.0, 7.6 Hz, 1H), 6.91 (s, 1H), 4.96-4.86 (m, 1H), 2.73-2.64 (m, 1H), 2.43-2.32 (m, 2H), 2.30-2.22 (m, 1H), 2.17 (s, 3H), 2.00-1.84 (m, 2H), 1.78-1.67 (m, 1H), 1.60-1.44 (m, 2H), 0.89-0.78 (m, 4H).

### Example 89: cis-N-(5-(2-cyano-5-(((1S,4S-4-hydroxy-4-methylcyclohexyl)oxy)pyridin-4-yl)pyrazolo[1,5-a]pyridin-2-yl)cyclopropanecarboxamide

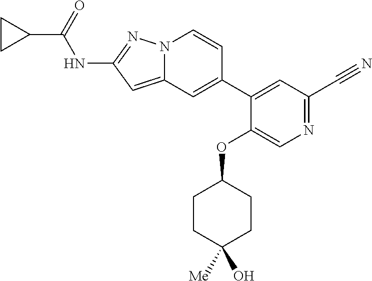 [SMILES: C[C@]1(O)CC[C@@H](Oc2cnc(C#N)cc2-c2ccn3nc(NC(=O)C4CC4)cc3c2)CC1]

The title compound (37 mg, 18%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (cis)-1-methylcyclohexane-1,4-diol was used instead of (R)-(1-methylpyrrolidin-3-yl)methanol. MS (ESI): mass calcd. for C₂₄H₂₅N₅O₃, 431.2. m/z found, 432.2 [M+H]⁺. ¹H NMR (400 MHz, DMSO-d₆) δ 11.13 (s, 1H), 8.70 (s, 1H), 8.62 (d, J=7.2 Hz, 1H), 8.17 (s, 1H), 7.91 (s, 1H), 7.15-7.06 (m, 1H), 6.92 (s, 1H), 4.81-4.67 (m, 1H), 4.21 (s, 1H), 2.01-1.90 (m, 1H), 1.89-1.68 (m, 4H) 1.63-1.53 (m, 2H), 1.52-1.35 (m, 2H), 1.12 (s, 3H), 0.92-0.76 (m, 4H).

### Example 90: N-[5-[2-cyano-5-(2-methyl-1,3,3a,4,6,6a-hexahydropyrrolo[3,4-c]pyrrol-5-yl)-4-pyridyl]pyrazolo[1,5-a]pyridin-2-yl]cyclopropanecarboxamide

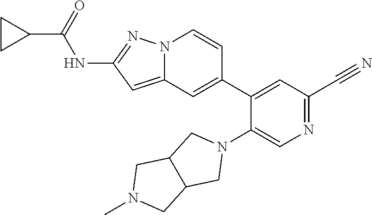 [SMILES: CN1CC2CN(c3cnc(C#N)cc3-c3ccn4nc(NC(=O)C5CC5)cc4c3)CC2C1]

The title compound (3.6 mg, 2.7%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and 2-methyloctahydropyrrolo[3,4-c]pyrrole was used instead of (R)-(1-methylpyrrolidin-3-yl)methanol. MS (ESI): mass calcd. for C₂₄H₂₅N₇O, 427.2. m/z found, 428.3 [M+H]⁺. ¹H NMR (400 MHz, DMSO-d₆) δ 11.11 (s, 1H), 8.59 (d, J=7.2 Hz, 1H), 8.33 (s, 1H), 7.78 (s, 1H), 7.73 (d, J=1.2 Hz, 1H), 6.94 (dd, J=2.0, 7.2 Hz, 1H), 6.89 (s, 1H), 3.26-3.19 (m, 2H), 3.01-2.94 (m, 2H), 2.75-2.65 (m, 2H), 2.43-2.37 (m, 2H), 2.31-2.26 (m, 2H), 2.20 (s, 3H), 1.98-1.90 (m, 1H), 0.86-0.79 (m, 4H).

### Example 91: N-[5-[2-cyano-5-(2-methyl-2,6-diazaspiro[3.3]heptan-6-yl)-4-pyridyl]pyrazolo[1,5-a]pyridin-2-yl]cyclopropanecarboxamide

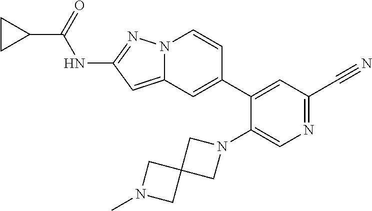 [SMILES: CN1CC2(C1)CN(c1cnc(C#N)cc1-c1ccn3nc(NC(=O)C4CC4)cc3c1)C2]

The title compound (4.2 mg, 3.1%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and 2-methyl-2,6-diazaspiro[3.3]heptane was used instead of (R)-(1-methylpyrrolidin-3-yl)methanol. MS (ESI): mass calcd. for C₂₃H₂₃N₇O, 413.2. m/z found, 414.2 [M+H]⁺. ¹H NMR (400 MHz, DMSO-d₆) δ 11.13 (s, 1H), 8.57 (d, J=6.8 Hz, 1H), 8.00 (s, 1H), 7.71 (s, 1H), 7.56 (d, J=1.2 Hz, 1H), 6.91 (s, 1H), 6.81 (dd, J=1.6, 6.8 Hz, 1H), 3.85 (s, 4H), 3.13 (s, 4H), 2.07 (s, 3H), 1.98-1.88 (m, 1H), 0.87-0.80 (m, 4H).

### Example 92: (3-endo)-N-[5-[2-cyano-5-[[(1R,5S)-3-oxa-9-azabicyclo[3.3.1]nonan-7-yl]oxy]-4-pyridyl]pyrazolo[1,5-a]pyridin-2-yl]cyclopropanecarboxamide

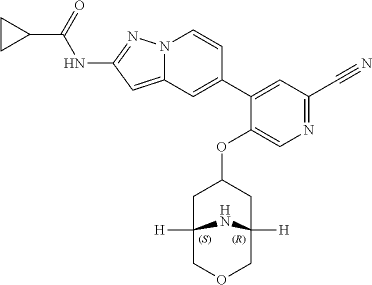

The title compound (166 mg, 60%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (1R,5S,7s)-3-oxa-9-azabicyclo[3.3.1]nonan-7-ol hydrochloride was used instead of (R)-(1-methylpyrrolidin-3-yl)methanol. MS (ESI): mass calcd. for C₂₄H₂₄N₆O₃, 444.2. m/z found, 445.3 [M+H]⁺. ¹H NMR (400 MHz, DMSO-d₆) δ 11.13 (s, 1H), 8.61-8.55 (m, 2H), 8.27 (s, 1H), 8.20 (s, 1H), 7.30-7.23 (m, 1H), 6.87 (s, 1H), 5.09-4.90 (m, 1H), 3.67-3.60 (m, 2H), 3.59-3.53 (m, 2H), 2.98-2.88 (m, 2H), 2.39-2.26 (m, 2H), 2.00-1.87 (m, 1H), 1.85-1.72 (m, 2H), 0.93-0.74 (m, 4H).

### Example 93: N-[5-[2-cyano-5-[(2,2,6,6-tetramethyl-4-piperidyl)oxy]-4-pyridyl]pyrazolo[1,5-a]pyridin-2-yl]cyclopropanecarboxamide

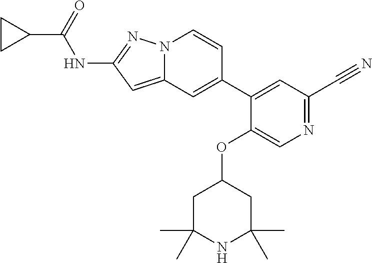 [SMILES: CC1(C)CC(Oc2cnc(C#N)cc2-c2ccn3nc(NC(=O)C4CC4)cc3c2)CC(C)(C)N1]

The title compound (69 mg, 32%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and 2,2,6,6-tetramethylpiperidin-4-ol was used instead of (R)-(1-methylpyrrolidin-3-yl)methanol. MS (ESI): mass calcd. for C₂₆H₃₀N₆O₂, 458.2. m/z found, 459.3 [M+H]⁺. ¹H NMR (400 MHz, DMSO-d₆) δ 11.14 (s, 1H), 8.71 (s, 1H), 8.61 (d, J=7.6 Hz, 1H), 8.18 (s, 1H), 7.89 (d, J=0.8 Hz, 1H), 7.07 (dd, J=2.0, 7.2 Hz, 1H), 6.93 (s, 1H), 5.32-5.17 (m, 1H), 2.10-1.99 (m, 2H), 1.98-1.89 (m, 1H), 1.26-1.16 (m, 8H), 1.09 (s, 6H), 0.87-0.77 (m, 4H).

### Example 94: N-[5-[2-cyano-5-[(2R)-2-hydroxypropoxy]-4-pyridyl]pyrazolo[1,5-a]pyridin-2-yl]cyclopropanecarboxamide

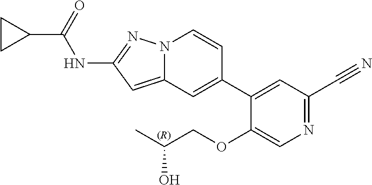

The title compound (19 mg, 11%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (R)-propane-1,2-diol was used instead of (R)-(1-methylpyrrolidin-3-yl)methanol. MS (ESI): mass calcd. for C₂₀H₁₉N₅O₃, 377.1. m/z found, 378.2 [M+H]⁺. ¹H NMR (400 MHz, DMSO-d₆) δ 11.16 (s, 1H), 8.68 (s, 1H), 8.61 (d, J=7.2 Hz, 1H), 8.19 (s, 1H), 8.02 (d, J=1.2 Hz, 1H), 7.18 (dd, J=1.6, 6.8 Hz, 1H), 6.92 (s, 1H), 5.05 (d, J=5.2 Hz, 1H), 4.19 (d, J=5.2 Hz, 2H), 4.11-3.88 (m, 1H), 2.01-1.88 (m, 1H), 1.15 (d, J=6.4 Hz, 3H), 0.91-0.77 (m, 4H).

### Example 95: N-[5-[2-cyano-5-[[(3S,5R)-5-(trifluoromethyl)-3-piperidyl]oxy]-4-pyridyl]pyrazolo[1,5-a]pyridin-2-yl]cyclopropanecarboxamide

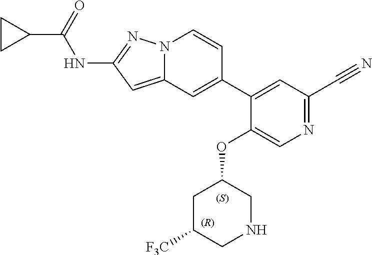

The title compound (52 mg, 27%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (3S,5R)-5-(trifluoromethyl)piperidin-3-ol was used instead of (R)-(1-methylpyrrolidin-3-yl)methanol. MS (ESI): mass calcd. for C₂₃H₂₁F₃N₆O₂, 470.2. m/z found, 471.2 [M+H]⁺. ¹H NMR (400 MHz, DMSO-d₆) δ 11.13 (s, 1H), 8.81 (s, 1H), 8.60 (d, J=7.6 Hz, 1H), 8.19 (s, 1H), 7.90 (d, J=1.2 Hz, 1H), 7.08 (dd, J=2.0, 7.2 Hz, 1H), 6.94 (s, 1H), 4.76-4.66 (m, 1H), 3.32-3.25 (m, 1H), 3.10-3.02 (m, 1H), 2.66-2.56 (m, 1H), 2.47-2.35 (m, 4H), 1.98-1.89 (m, 1H), 1.53-1.41 (m, 1H), 0.86-0.80 (m, 4H). ¹⁹F NMR (376 MHz, DMSO-d₆) δ −70.90 (d, J=8.6 Hz, 3F).

### Example 96: N-[5-[2-cyano-5-[(2R)-3,3,3-trifluoro-2-hydroxy-propoxy]-4-pyridyl]pyrazolo[1,5-a]pyridin-2-yl]cyclopropanecarboxamide

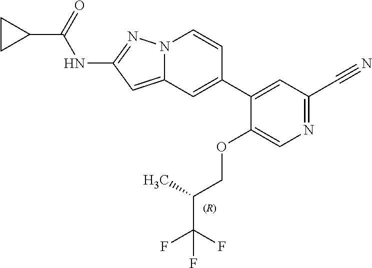

The title compound (47 mg, 11%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (R)-3,3,3-trifluoropropane-1,2-diol was used instead of (R)-(1-methylpyrrolidin-3-yl)methanol. MS (ESI): mass calcd. for C₂₀H₁₆F₃N₅O₃, 431.1. m/z found, 432.2 [M+H]⁺. ¹H NMR (400 MHz, DMSO-d₆) δ 11.15 (s, 1H), 8.75 (s, 1H), 8.60 (d, J=7.6 Hz, 1H), 8.23 (s, 1H), 8.01 (d, J=1.2 Hz, 1H), 7.16 (dd, J=2.0, 7.2 Hz, 1H), 6.93 (s, 1H), 6.81 (d, J=2.8 Hz, 1H), 4.64-4.33 (m, 3H), 1.98-1.86 (m, 1H), 0.91-0.75 (m, 4H). ¹⁹F NMR (376 MHz, DMSO-d₆) δ −75.84 (d, J=7.1 Hz, 3F).

### Example 97: N-[5-[2-cyano-5-[(2S)-3,3,3-trifluoro-2-hydroxy-propoxy]-4-pyridyl]pyrazolo[1,5-a]pyridin-2-yl]cyclopropanecarboxamide

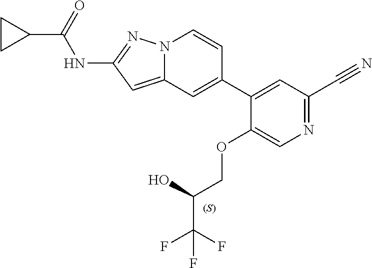

The title compound (34 mg, 8%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (S)-3,3,3-trifluoropropane-1,2-diol was used instead of (R)-(1-methylpyrrolidin-3-yl)methanol. MS (ESI): mass calcd. for C₂₀H₁₆F₃N₅O₃, 431.1. m/z found, 432.2 [M+H]⁺. ¹H NMR (400 MHz, DMSO-d₆) δ 11.14 (s, 1H), 8.74 (s, 1H), 8.59 (d, J=6.8 Hz, 1H), 8.22 (s, 1H), 8.01 (s, 1H), 7.20-7.10 (m, 1H), 6.93 (s, 1H), 6.82 (br s., 1H), 4.62-4.41 (m, 3H), 2.06-1.86 (m, 1H), 0.98-0.68 (m, 4H). ¹⁹F NMR (376 MHz, DMSO-d₆) δ −75.83 (d, J=6.8 Hz, 3F).

### Example 98: N-(5-(2-cyano-5-(((1s,3s)-3-(methylamino)cyclobutyl)methoxy)pyridin-4-yl)pyrazolo[1,5-a]pyridin-2-yl)cyclopropanecarboxamide

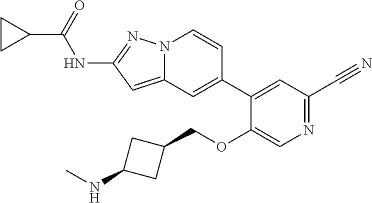 [SMILES: CN[C@H]1C[C@@H](COc2cnc(C#N)cc2-c2ccn3nc(NC(=O)C4CC4)cc3c2)C1]

The title compound (57 mg, 16%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and ((cis)-3-(methylamino)cyclobutyl)methanol was used instead of (R)-(1-methylpyrrolidin-3-yl)methanol. MS (ESI): mass calcd. for C₂₃H₂₄N₆O₂, 416.2. m/z found, 417.2 [M+H]⁺. ¹H NMR (400 MHz, DMSO-d₆) δ 11.15 (s, 1H), 8.66-8.60 (m, 2H), 8.18 (s, 1H), 7.93 (d, J=1.2 Hz, 1H), 7.10 (dd, J=2.0, 7.2 Hz, 1H), 6.92 (s, 1H), 4.25 (d, J=6.4 Hz, 2H), 3.02-2.92 (m, 1H), 2.40-2.29 (m, 1H), 2.27-2.18 (m, 2H), 2.11 (s, 3H), 1.98-1.89 (m, 1H), 1.57-1.45 (m, 2H), 0.89-0.76 (m, 4H).

### Example 99: N-[5-[2-cyano-5-[3,3,3-trifluoro-2-hydroxy-2-(trifluoromethyl)propoxy]-4-pyridyl]pyrazolo[1,5-a]pyridin-2-yl]cyclopropanecarboxamide

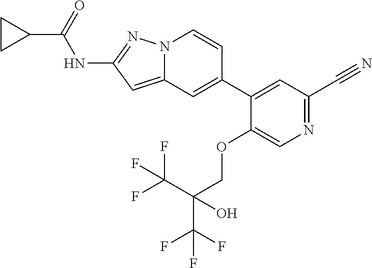 [SMILES: N#Cc1cc(-c2ccn3nc(NC(=O)C4CC4)cc3c2)c(OCC(O)(C(F)(F)F)C(F)(F)F)cn1]

The title compound (68 mg, 6.7%) was prepared as described in Example 1 where N-(5-(2-cyano-5-fluoropyridin-4-yl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and 3,3,3-trifluoro-2-(trifluoromethyl)propane-1,2-diol was used instead of (R)-(1-methylpyrrolidin-3-yl)methanol. MS (ESI): mass calcd. for C₂₁H₁₅F₆N₅O₃, 499.1. m/z found, 500.1 [M+H]⁺. ¹H NMR (400 MHz, DMSO-d₆) δ 11.16 (s, 1H), 8.82 (s, 1H), 8.62 (d, J=7.2 Hz, 1H), 8.59 (s, 1H), 8.24 (s, 1H), 7.92 (d, J=0.8 Hz, 1H), 7.08 (dd, J=2.0, 7.6 Hz, 1H), 6.89 (s, 1H), 4.81 (s, 2H), 1.99-1.90 (m, 1H), 0.87-0.79 (m, 4H). ¹⁹F NMR (376 MHz, DMSO-d₆) δ −74.49 (s, 6F).

### Example 100: N-[5-[5-cyano-2-[[(3R)-1-methylpyrrolidin-3-yl]methoxy]phenyl]pyrazolo[1,5-a]pyridin-2-yl]cyclopropanecarboxamide

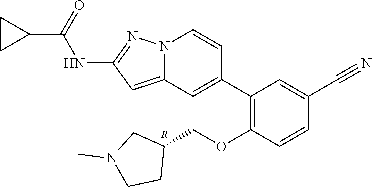

The title compound (83.3 mg, 32%) was prepared as described in Example 1 where N-(5-(5-cyano-2-fluorophenyl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide. MS (ESI): mass calcd. for C₂₄H₂₅N₅O₂, 415.2. m/z found, 416.2 [M+H]⁺. ¹H NMR (400 MHz, MeOH-d4) δ 8.37 (d, J=7.2 Hz, 1H), 7.76-7.68 (m, 2H), 7.65-7.57 (m, 1H), 7.22 (d, J=8.4 Hz, 1H), 6.93 (dd, J=7.2, 2.0 Hz, 1H), 6.86 (s, 1H), 4.15-3.97 (m, 2H), 2.76-2.53 (m, 3H), 2.49-2.39 (m, 1H), 2.37-2.31 (m, 1H), 2.29 (s, 3H), 2.07-1.95 (m, 1H), 1.92-1.81 (m, 1H), 1.68-1.54 (m, 1H), 1.03-0.96 (m, 2H), 0.93-0.85 (m, 2H).

### Example 101: N-[5-[5-cyano-2-[[(2R)-morpholin-2-yl]methoxy]phenyl]pyrazolo[1,5-a]pyridin-2-yl]cyclopropanecarboxamide

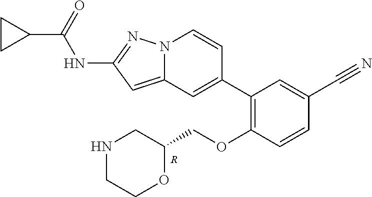

The title compound (529 mg, 81%) was prepared as described in Example 1 where N-(5-(5-cyano-2-fluorophenyl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and [(2R)-morpholin-2-yl]methanol was used instead of (R)-(1-methylpyrrolidin-3-yl)methanol. MS (ESI): mass calcd. for C₂₃H₂₃N₅O₃, 417.2. m/z found, 418.2 [M+H]⁺. ¹H NMR (500 MHz, MeOH-d₄) δ 8.41-8.35 (m, 1H), 7.78 (d, J=2.1 Hz, 1H), 7.76-7.71 (m, 2H), 7.24 (d, J=8.6 Hz, 1H), 7.01 (dd, J=7.3, 2.0 Hz, 1H), 6.87 (s, 1H), 4.15-4.11 (m, 2H), 3.91-3.86 (m, 1H), 3.84-3.77 (m, 1H), 3.68-3.56 (m, 1H), 2.87 (dd, J=12.5, 2.5 Hz, 1H), 2.80-2.73 (m, 2H), 2.70-2.63 (m, 1H), 1.90-1.81 (m, 1H), 1.04-0.96 (m, 2H), 0.93-0.86 (m, 2H).

### Example 102: N-[5-[5-cyano-2-[[(2R)-6,6-dimethylmorpholin-2-yl]methoxy]phenyl]pyrazolo[1,5-a]pyridin-2-yl]cyclopropanecarboxamide

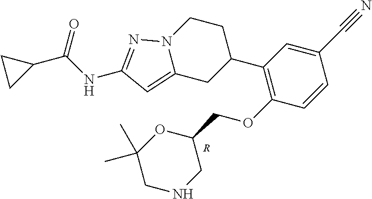

The title compound (266 mg, 96%) was prepared as described in Example 1 where N-(5-(5-cyano-2-fluorophenyl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (R)-(6,6-dimethylmorpholin-2-yl)methanol was used instead of (R)-(1-methylpyrrolidin-3-yl)methanol. MS (ESI): mass calcd. for C₂₅H₂₇N₅O₃, 445.2. m/z found, 446.2 [M+H]⁺. ¹H NMR (400 MHz, DMSO-d₆) δ 11.06 (s, 1H), 8.53 (dd, J=7.3, 1.0 Hz, 1H), 7.93 (d, J=2.1 Hz, 1H), 7.88-7.81 (m, 2H), 7.30 (d, J=8.7 Hz, 1H), 7.09 (dd, J=7.3, 2.0 Hz, 1H), 6.87 (s, 1H), 4.06-4.02 (m, 2H), 3.96-3.88 (m, 1H), 2.81-2.74 (m, 1H), 2.55 (d, J=12.2 Hz, 1H), 2.42-2.29 (m, 2H), 1.97-1.88 (m, 1H), 1.23 (s, 3H), 1.09 (s, 3H), 0.87-0.76 (m, 4H).

### Example 103: N-[5-[5-cyano-2-[[(2S)-morpholin-2-yl]methoxy]phenyl]pyrazolo[1,5-a]pyridin-2-yl]cyclopropanecarboxamide

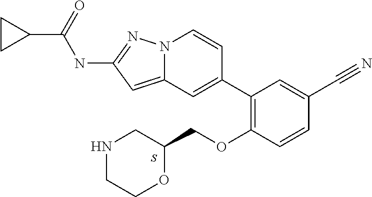

The title compound (434 mg, 76%) was prepared as described in Example 1 where N-(5-(5-cyano-2-fluorophenyl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and [(2S)-morpholin-2-yl]methanol was used instead of (R)-(1-methylpyrrolidin-3-yl)methanol. MS (ESI): mass calcd. for C₂₃H₂₃N₅O₃, 417.2. m/z found, 418.2 [M+H]⁺. ¹H NMR (400 MHz, MeOH-d4) δ 8.42-8.34 (m, 1H), 7.78 (d, J=2.1 Hz, 1H), 7.75-7.68 (m, 2H), 7.24 (d, J=8.6 Hz, 1H), 7.01 (dd, J=7.3, 2.0 Hz, 1H), 6.87 (s, 1H), 4.15-4.11 (m, 2H), 3.93-3.85 (m, 1H), 3.84-3.75 (m, 1H), 3.67-3.55 (m, 1H), 2.87 (dd, J=12.5, 2.5 Hz, 1H), 2.80-2.72 (m, 2H), 2.71-2.62 (m, 1H), 1.92-1.84 (m, 1H), 1.04-0.96 (m, 2H), 0.93-0.84 (m, 2H).

### Example 104: N-[5-[5-cyano-2-[[(2S)-6,6-dimethylmorpholin-2-yl]methoxy]phenyl]pyrazolo[1,5-a]pyridin-2-yl]cyclopropanecarboxamide

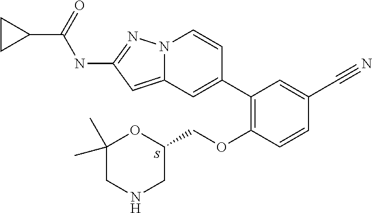

The title compound (87 mg, 31%) was prepared as described in Example 1 where N-(5-(5-cyano-2-fluorophenyl)pyrazolo[1,5-a]pyridin-2-yl)cyclopropanecarboxamide was used instead of N-(5-(5-chloro-2-cyanopyridin-4-yl)pyrazolo[1,5-a]pyridin-2-yl)cyclopropanecarboxamide and (S)-(6,6-dimethylmorpholin-2-yl)methanol HCL was used instead of (R)-(1-methylpyrrolidin-3-yl)methanol. MS (ESI): mass calcd. for C₂₅H₂₇N₅O₃, 445.2. m/z found, 446.2 [M+H]⁺. ¹H NMR (500 MHz, DMSO-d₆) δ 11.07 (s, 1H), 8.57-8.51 (m, 1H), 7.93 (d, J=2.1 Hz, 1H), 7.87-7.82 (m, 2H), 7.30 (d, J=8.7 Hz, 1H), 7.08 (dd, J=7.3, 2.0 Hz, 1H), 6.88 (s, 1H), 4.11-4.02 (m, 2H), 3.99-3.91 (m, 1H), 2.88-2.81 (m, 1H), 2.64 (d, J=12.3 Hz, 1H), 2.46-2.35 (m, 2H), 1.97-1.90 (m, 1H), 1.24 (s, 3H), 1.11 (s, 3H), 0.88-0.79 (m, 4H).

### Example 105: 5-[[(3R)-1-methylpyrrolidin-3-yl]methoxy]-4-[2-(4-pyridylamino)pyrazolo[1,5-a]pyridin-5-yl]pyridine-2-carbonitrile

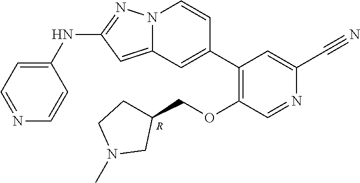

The title compound (49 mg, 26%) was prepared as described in Example 4 where 4-chloropyridine was used instead of 3-bromopyridine. MS (ESI): mass calcd. for C₂₄H₂₃N₇O, 425.2. m/z found, 426.1 [M+H]⁺. ¹H NMR (400 MHz, DMSO-d₆) δ 9.67 (s, 1H), 8.70-8.64 (m, 2H), 8.30 (d, J=6.4 Hz, 2H), 8.19 (s, 1H), 7.86 (s, 1H), 7.47 (d, J=6.4 Hz, 2H), 7.04 (dd, J=1.6, 7.2 Hz, 1H), 6.27 (s, 1H), 4.27-4.17 (m, 2H), 2.58-2.53 (m, 2H), 2.41 (m, 2H), 2.36-2.27 (m, 1H), 2.21 (s, 3H), 1.97-1.85 (m, 1H), 1.59-1.49 (m, 1H).

### Example 106: 4-(2-anilinopyrazolo[1,5-a]pyridin-5-yl)-5-[[(3R)-1-methylpyrrolidin-3-yl]methoxy]pyridine-2-carbonitrile

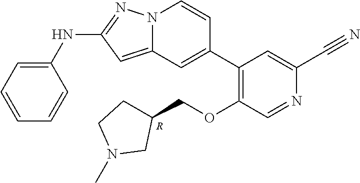

The title compound (220 mg, 90%) was prepared as described in Example 4 where bromobenzene was used instead of 3-bromopyridine. MS (ESI): mass calcd. for C₂₅H₂₄N₆O, 424.2. m/z found, 425.2 [M+H]⁺. ¹H NMR (400 MHz, DMSO-d₆) δ 9.01 (s, 1H), 8.66 (s, 1H), 8.60 (d, J=7.2 Hz, 1H), 8.17 (s, 1H), 7.81-7.76 (m, 1H), 7.53 (d, J=8.0 Hz, 2H), 7.31-7.23 (m, 2H), 6.96 (dd, J=1.6, 7.2 Hz, 1H), 6.86-6.79 (m, 1H), 6.16 (s, 1H), 4.26-4.16 (m, 2H), 2.61-2.54 (m, 2H), 2.44-2.38 (m, 2H), 2.36-2.28 (m, 1H), 2.22 (s, 3H), 1.98-1.86 (m, 1H), 1.59-1.49 (m, 1H).

### Example 107: 5-[[(3R)-1-methylpyrrolidin-3-yl]methoxy]-4-[2-(2-pyridylamino)pyrazolo[1,5-a]pyridin-5-yl]pyridine-2-carbonitrile

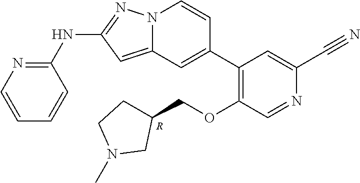

The title compound (119 mg, 48%) was prepared as described in Example 4 where 2-bromopyridine was used instead of 3-bromopyridine. MS (ESI): mass calcd. for C₂₄H₂₃N₇O, 425.2. m/z found, 426.2 [M+H]⁺. ¹H NMR (400 MHz, DMSO-d₆) δ 9.85 (s, 1H), 8.68 (s, 1H), 8.61 (d, J=6.8 Hz, 1H), 8.23 (d, J=3.6 Hz, 1H), 8.20 (s, 1H), 7.86 (s, 1H), 7.67-7.59 (m, 1H), 7.32 (d, J=8.4 Hz, 1H), 7.01 (dd, J=1.6, 7.2 Hz, 1H), 6.94 (s, 1H), 6.86-6.75 (m, 1H), 4.44-4.28 (m, 2H), 3.55-3.36 (m, 3H), 3.19-3.05 (m, 1H), 2.96-2.86 (m, 1H), 2.81 (s, 3H), 2.22-2.10 (m, 1H), 1.90-1.77 (m, 1H).

### Example 108: (S)—N-[5-[2-cyano-5-[(3S)-1-(2-methoxyethyl)pyrrolidin-3-yl]oxy-4-pyridyl]pyrazolo[1,5-a]pyridin-2-yl]cyclopropanecarboxamide

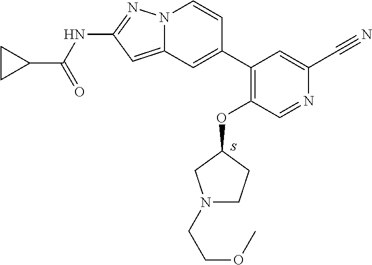

The title compound (10 mg, 18%) was prepared as described in Example 5 where 1-bromo-2-methoxyethane was used instead of 2,2,2-trifluoroethyl trifluoromethanesulfonate. MS (ESI): mass calcd. for C₂₄H₂₆N₆O₃, 446.2. m/z found, 447.2 [M+H]⁺. ¹H NMR (400 MHz, DMSO-d₆) δ 11.14 (s, 1H), 8.63-8.58 (m, 2H), 8.19 (s, 1H), 7.96 (s, 1H), 7.13 (dd, J=1.6, 7.2 Hz, 1H), 6.93 (s, 1H), 5.31-5.23 (m, 1H), 3.44-3.40 (m, 2H), 3.21 (s, 3H), 3.00-2.87 (m, 1H), 2.87-2.75 (m, 2H), 2.69-2.58 (m, 2H), 2.48-2.42 (m, 1H), 2.42-2.28 (m, 1H), 1.99-1.90 (m, 1H), 1.89-1.79 (m, 1H), 0.86-0.80 (m, 4H).

### Example 109: (S)—N-[5-[2-cyano-5-[(3S)-1-(2-pyridylmethyl)pyrrolidin-3-yl]oxy-4-pyridyl]pyrazolo[1,5-a]pyridin-2-yl]cyclopropanecarboxamide

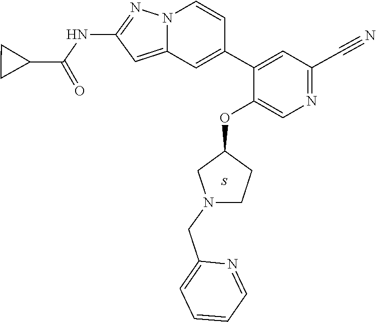

The title compound (22.5 mg, 35%) was prepared as described in Example 5 where 2-(bromomethyl)pyridine hydrobromide was used instead of 2,2,2-trifluoroethyl trifluoromethanesulfonate. MS (ESI): mass calcd. for C₂₇H₂₅N₇O₂, 479.2. m/z found, 480.3 [M+H]⁺. ¹H NMR (400 MHz, DMSO-d₆) δ 11.16 (s, 1H), 8.64-8.59 (m, 2H), 8.47 (d, J=4.4 Hz, 1H), 8.20 (s, 1H), 7.97-7.94 (m, 1H), 7.74-7.67 (m, 1H), 7.38 (d, J=8.0 Hz, 1H), 7.25-7.20 (m, 1H), 7.14 (dd, J=1.6, 7.2 Hz, 1H), 6.94 (s, 1H), 5.33-5.27 (m, 1H), 3.80-3.67 (m, 2H), 2.93-2.79 (m, 3H), 2.49-2.46 (m, 1H), 2.45-2.35 (m, 1H), 1.99-1.92 (m, 1H), 1.92-1.82 (m, 1H), 0.87-0.81 (m, 4H).

### Example 110: (R)—N-[5-[2-cyano-5-[(3R)-1-(2,2,2-trifluoroethyl)pyrrolidin-3-yl]oxy-4-pyridyl]pyrazolo[1,5-a]pyridin-2-yl]cyclopropanecarboxamide

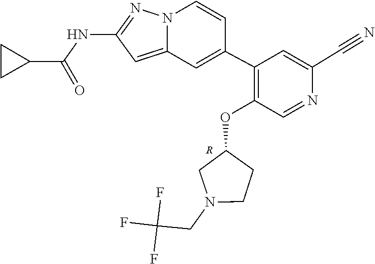

The title compound (65 mg, 53%) was prepared as described in Example 5, using (R)-pyrrolidin-3-ol in place of (S)-pyrrolidin-3-ol. MS (ESI): mass calcd. for C₂₃H₂₁F₃N₆O₂, 470.2. m/z found, 471.2 [M+H]⁺. ¹H NMR (400 MHz, DMSO-d₆) δ 11.13 (s, 1H), 8.63 (s, 1H), 8.58 (d, J=7.6 Hz, 1H), 8.20 (s, 1H), 7.97-7.94 (m, 1H), 7.13 (dd, J=2.0, 7.6 Hz, 1H), 6.92 (s, 1H), 5.35-5.29 (m, 1H), 3.33-3.24 (m, 2H), 3.17-3.10 (m, 1H), 2.98-2.90 (m, 2H), 2.75-2.68 (m, 1H), 2.40-2.29 (m, 1H), 1.98-1.85 (m, 2H), 0.86-0.79 (m, 4H). ¹⁹F NMR (376 MHz, DMSO-d₆) δ −68.88 (t, J=10.5 Hz, 3F).

### Example 111: (R)—N-[5-[2-cyano-5-[(3R)-1-(2-methoxyethyl)pyrrolidin-3-yl]oxy-4-pyridyl]pyrazolo[1,5-a]pyridin-2-yl]cyclopropanecarboxamide

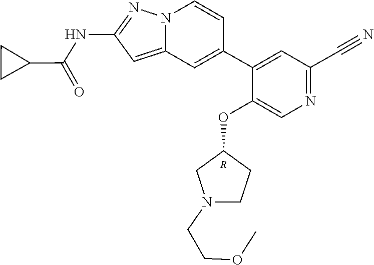

The title compound (58 mg, 49%) was prepared as described in Example 5 where 1-bromo-2-methoxyethane was used instead of (2,2,2-trifluoroethyl trifluoromethanesulfonate. MS (ESI): mass calcd. for C₂₄H₂₆N₆O₃, 446.2. m/z found, 447.2 [M+H]⁺. ¹H NMR (400 MHz, DMSO-d₆) δ 11.13 (s, 1H), 8.64-8.58 (m, 2H), 8.19 (s, 1H), 7.95 (d, J=0.8 Hz, 1H), 7.13 (dd, J=1.6, 7.2 Hz, 1H), 6.93 (s, 1H), 5.30-5.23 (m, 1H), 3.41 (t, J=6.0 Hz, 2H), 3.21 (s, 3H), 2.96-2.86 (m, 1H), 2.83-2.73 (m, 2H), 2.64-2.56 (m, 2H), 2.48-2.41 (m, 1H), 2.39-2.27 (m, 1H), 1.99-1.89 (m, 1H), 1.87-1.77 (m, 1H), 0.86-0.79 (m, 4H).

### Example 112: (R)—N-[5-[2-cyano-5-[(3R)-1-(2-pyridylmethyl)pyrrolidin-3-yl]oxy-4-pyridyl]pyrazolo[1,5-a]pyridin-2-yl]cyclopropanecarboxamide

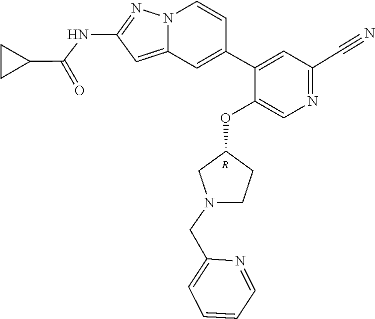

The title compound (48 mg, 38%) was prepared as described in Example 5 where 2-(bromomethyl)pyridine hydrobromide was used instead of (2,2,2-trifluoroethyl trifluoromethanesulfonate. MS (ESI): mass calcd. for C₂₇H₂₅N₇O₂, 479.2. m/z found, 480.2 [M+H]⁺. ¹H NMR (400 MHz, DMSO-d₆) δ 11.16 (s, 1H), 8.64-8.59 (m, 2H), 8.47 (d, J=4.0 Hz, 1H), 8.20 (s, 1H), 7.97-7.94 (m, 1H), 7.74-7.68 (m, 1H), 7.38 (d, J=7.6 Hz, 1H), 7.26-7.20 (m, 1H), 7.14 (dd, J=2.0, 7.6 Hz, 1H), 6.94 (s, 1H), 5.33-5.27 (m, 1H), 3.80-3.67 (m, 2H), 2.92-2.80 (m, 3H), 2.49-2.35 (m, 2H), 1.99-1.92 (m, 1H), 1.92-1.82 (m, 1H), 0.88-0.80 (m, 4H).

### Example 113: (R)—N-[5-[2-cyano-5-[(3R)-1-(cyanomethyl)pyrrolidin-3-yl]oxy-4-pyridyl]pyrazolo[1,5-a]pyridin-2-yl]cyclopropanecarboxamide

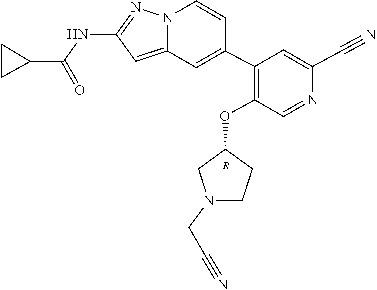

The title compound (32 mg, 54%) was prepared as described in Example 5 where 2-bromoacetonitrile was used instead of (2,2,2-trifluoroethyl trifluoromethanesulfonate. MS (ESI): mass calcd. for C₂₃H₂₁N₇O₂, 427.2. m/z found, 428.2 [M+H]⁺. ¹H NMR (400 MHz, DMSO-d₆) δ 11.14 (s, 1H), 8.61 (s, 1H), 8.58 (d, J=7.2 Hz, 1H), 8.18 (s, 1H), 7.92 (d, J=0.8 Hz, 1H), 7.11 (dd, J=2.0, 7.2 Hz, 1H), 6.92 (s, 1H), 5.36-5.30 (m, 1H), 3.86 (s, 2H), 3.05-2.97 (m, 1H), 2.87-2.79 (m, 2H), 2.60-2.53 (m, 1H), 2.46-2.35 (m, 1H), 1.97-1.85 (m, 2H), 0.86-0.79 (m, 4H).

### Example 114: (S)—N-[5-[2-cyano-5-[(3S)-1-(cyanomethyl)pyrrolidin-3-yl]oxy-4-pyridyl]pyrazolo[1,5-a]pyridin-2-yl]cyclopropanecarboxamide

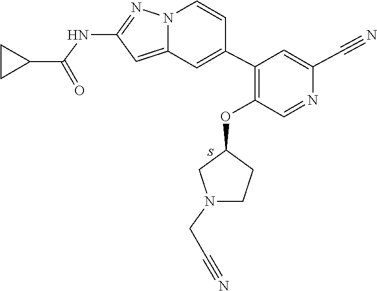

The title compound (67 mg, 38%) was prepared as described in Example 5 where 2-bromoacetonitrile was used instead of (2,2,2-trifluoroethyl trifluoromethanesulfonate. MS (ESI): mass calcd. for C₂₃H₂₁N₇O₂, 427.2. m/z found, 428.1 [M+H]⁺. ¹H NMR (400 MHz, DMSO-d₆) δ 11.14 (s, 1H), 8.62 (s, 1H), 8.59 (d, J=7.2 Hz, 1H), 8.20 (s, 1H), 7.93 (d, J=1.2 Hz, 1H), 7.11 (dd, J=2.0, 7.6 Hz, 1H), 6.93 (s, 1H), 5.37-5.30 (m, 1H), 3.87 (s, 2H), 3.05-2.98 (m, 1H), 2.87-2.81 (m, 2H), 2.60-2.54 (m, 1H), 2.46-2.38 (m, 1H), 1.98-1.88 (m, 2H), 0.86-0.78 (m, 4H).

### Example 115: N-(5-(2-methyl-5-((1-methylazetidin-3-yl)oxy)pyridin-4-yl)pyrazolo[1,5-a]pyridin-2-yl)cyclopropanecarboxamide

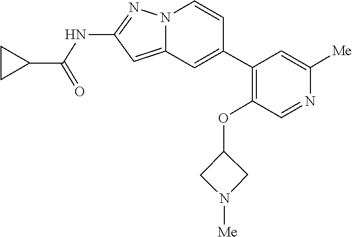 [SMILES: Cc1cc(-c2ccn3nc(NC(=O)C4CC4)cc3c2)c(OC2CN(C)C2)cn1]

To a solution consisting of 1-methylazetidin-3-ol (7.87 g, 90.3 mmol) and DMA (125 mL) was treated with sodium hydride, 60% dispersion in mineral oil (4.66 g, 117 mmol) and stirred for 2 min at rt. 5-(5-fluoro-2-methylpyridin-4-yl)pyrazolo[1,5-a]pyridin-2-amine (Intermediate 6, 10.4 g, 42.7 mmol) was added as a solution in DMA (50 mL) and the mixture placed on a pre-heated block at 90° C. for 90 min. The reaction mixture was cooled to room temperature and treated with water (500 mL). The resulting precipitate was collected by filtration, dried under vacuum, and purified by flash column chromatography (SiO₂, 50-100% EtOAc/hexanes. Fractions containing product were combined and concentrated to give the title compound (9.8 g, 74%) as a yellow foam. MS (ESI): mass calcd. for C₂₁H₂₃N₅O₂, 377.19. m/z found, 310.2 [M+H]⁺. ¹H NMR (400 MHz, DMSO-d6) δ 11.08 (s, 1H), 8.64-8.49 (m, 1H), 8.06 (s, 1H), 7.88-7.75 (m, 1H), 7.37 (s, 1H), 7.06 (dd, J=7.2, 2.0 Hz, 1H), 6.90 (s, 1H), 4.97-4.80 (m, 1H), 3.82-3.62 (m, 2H), 3.06-2.87 (m, 2H), 2.45 (s, 3H), 2.27 (s, 3H), 1.98-1.90 (m, 1H), 0.88-0.76 (m, 4H).

### Example 116: N-(1-(difluoromethyl)-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine

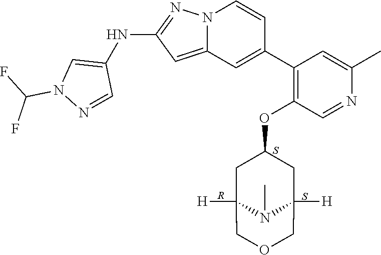

N-(1-(difluoromethyl)-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine is prepared by a three step process. In a first step, step A as shown below provided tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate. tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate is then subjected to the conditions described in Example 117, steps B and C as the second and third steps to provide N-(1-(difluoromethyl)-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine.

Step A: tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate. To a vial containing tert-butyl (1R,5S,7s)-7-((4-(2-aminopyrazolo[1,5-a]pyridin-5-yl)-6-methylpyridin-3-yl)oxy)-3-oxa-9-azabicyclo[3.3.1]nonane-9-carboxylate (Intermediate 9, 140 mg, 0.301 mmol), 4-bromo-1-(difluoromethyl)-1H-pyrazole (88.9 mg, 0.451 mmol), tert-BuBrettPhos-Pd-G3, [(2-Di-tert-butylphosphino-3,6-dimethoxy-2′,4′,6′-triisopropyl-1,1′-biphenyl)-2-(2′-amino-1,1′-biphenyl)]palladium(II) methanesulfonate (25.7 mg, 0.030 mmol, 10 mol %), and cesium carbonate (367 mg, 1.13 mmol) was added 1,4-dioxane (3.0 mL, 1M) via syringe under N₂ atmosphere. The reaction mixture was evacuated with sonication for 2 minutes then vented to Argon. The resulting vial was heated in a microwave reactor at 120° C. for 1 hour. The reaction mixture was filtered through diatomaceous earth and concentrated in vacuo. The residue was purified by flash column chromatography (10-100% EtOAc in hexanes) and the fractions containing product were combined and concentrated to afford the title compound (130 mg, 74%) as a beige foam. MS (ESI): mass calcd. for C₂₉H₃₃N₇O₄F₂, 581.6. m/z found, 582.3 [M+H]⁺.

### Example 117: 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine

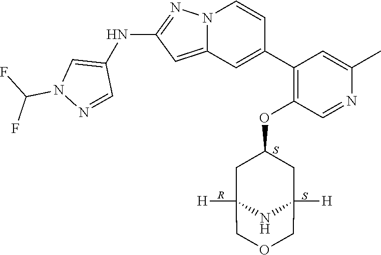

5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine.

Step B: To a vial containing tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate (130 mg, 0.224 mmol) was added trifluoracetic acid (0.77 mL, 10.1 mmol) at 23° C. and after 10 minutes the solvent was removed in vacuo. The residue was diluted with dichloromethane (6 mL) followed by saturated aq. K₂CO₃ (3 mL). The layers separated and the aqueous was extracted with dichloromethane (3×2 mL). The combined organics were dried with MgSO₄, filtered and concentrated in vacuo to give the title compound (90 mg, 83%) as a white solid. MS (ESI): mass calcd. for C₂₄H₂₅N₇O₂F₂, 481.5. m/z found, 482.2 [M+H]⁺. ¹H NMR (500 MHz, DMSO-d6) δ 8.90 (s, 1H), 8.52-8.48 (m, 1H), 8.28 (s, 1H), 8.24 (d, J=0.7 Hz, 1H), 7.87-7.84 (m, 1H), 7.76-7.72 (m, 2H), 7.35 (s, 1H), 6.99 (dd, J=7.2, 2.0 Hz, 1H), 5.97 (s, 1H), 4.68-4.60 (m, 1H), 3.56 (dd, J=10.6, 2.3 Hz, 2H), 3.50 (d, J=10.6 Hz, 2H), 2.92 (d, J=8.6 Hz, 2H), 2.45 (s, 3H), 2.32-2.24 (m, 2H), 1.68-1.60 (m, 2H).

Step C: N-(1-(difluoromethyl)-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine. To a solution of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine (crude, 0.224 mmol) in MeOH (4.7 mL, 0.04 M) was added H(CHO)ₙ (25.3 mg, 0.28 mmol) followed by NaCNBH₃ (35.2 mg, 0.581 mmol) at 23° C. After 1 h, saturated aq. NaHCO₃ (2.5 mL) and water (2.5 mL) was added followed by EtOAc (10 mL). The layers were separated and the aqueous was extracted with EtOAc (3×10 mL) and the combined organics were concentrated in vacuo. The reaction mixture was purified by preparative HPLC using a Waters Xbridge Prep OBD C18 50×100 mm×5 μm column (eluent: 10% to 100% (v/v) CH₃CN and H₂O with 20 mM NH₄HCO₃) to afford pure product. The product was suspended in water/MeCN (1:1, 10 mL), the mixture frozen using dry ice/acetone, and then lyophilized to dryness to afford N-(1-(difluoromethyl)-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine (25 mg, 27%) as a white solid. MS (ESI): mass calcd. for C₂₅H₂₇N₇O₂F₂, 496.2. m/z found, 495.22 [M+H]⁺. ¹H NMR (500 MHz, DMSO-d6) δ 8.88 (s, 1H), 8.48 (dt, J=7.3, 0.9 Hz, 1H), 8.24 (d, J=0.7 Hz, 1H), 8.18 (s, 1H), 8.03 (dd, J=2.0, 0.9 Hz, 1H), 7.75-7.72 (m, 2H), 7.37 (s, 1H), 7.08 (dd, J=7.3, 2.0 Hz, 1H), 5.93 (s, 1H), 4.71-4.63 (m, 1H), 3.79 (dd, J=10.8, 2.5 Hz, 2H), 3.53-3.45 (m, 2H), 2.63 (d, J=7.0 Hz, 2H), 2.45 (s, 3H), 2.43-2.34 (m, 5H), 1.67-1.59 (m, 2H).

### Example 118: N-(4-fluoro-1-methyl-1H-pyrazol-3-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine

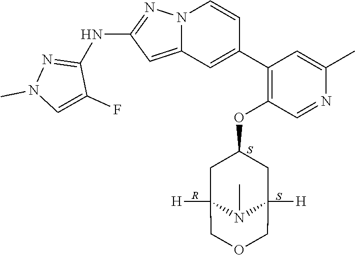

The title compound was prepared using the reductive amination method analogous to that described in Example 164, and using Example 399 instead of N-(5-(5-(((1S,4S,7R)-2-oxa-5-azabicyclo[2.2.1]heptan-7-yl)oxy)-2-methylpyridin-4-yl)pyrazolo[1,5-a]pyridin-2-yl)cyclopropanecarboxamide. MS (ESI): mass calcd. for C₂₅H₂₈FN₇O₂, 477.2. m/z found, 478.3 [M+H]⁺. ¹H NMR (500 MHz, DMSO-d₆) δ 8.75 (s, 1H), 8.38 (dd, J=7.2, 0.9 Hz, 1H), 8.18 (s, 1H), 8.02 (dd, J=2.0, 0.9 Hz, 1H), 7.75 (d, J=4.6 Hz, 1H), 7.36 (s, 1H), 7.09 (dd, J=7.2, 2.1 Hz, 1H), 6.26 (d, J=0.8 Hz, 1H), 4.70-4.62 (m, 1H), 3.78 (dd, J=10.8, 2.4 Hz, 2H), 3.70 (s, 3H), 3.48 (d, J=10.7 Hz, 2H), 2.65-2.60 (m, 2H), 2.44 (s, 3H), 2.39 (s, 3H), 2.38-2.32 (m, 1H), 1.68-1.58 (m, 2H).

### Example 119: N-(3-fluoro-1-methyl-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine

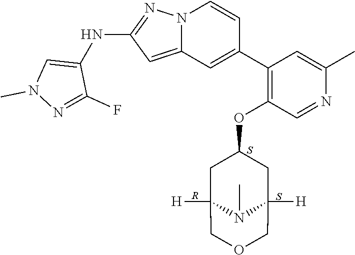

The title compound (19.8 mg, 15%) was prepared as described in Example 116 steps A-C where ((4-bromo-3-fluoro-1-methyl-1H-pyrazole)) was used instead of 4-bromo-1-(difluoromethyl)-1H-pyrazole in step A. MS (ESI): mass calcd. for C₂₅H₂₈FN₇O₂, 477.2. m/z found, 478.3 [M+H]⁺. ¹H NMR (500 MHz, DMSO-d₆) δ 8.38 (d, J=7.2 Hz, 1H), 8.18 (s, 1H), 8.06 (s, 1H), 8.00 (dd, J=2.1, 0.9 Hz, 1H), 7.36 (s, 1H), 7.04 (dd, J=7.2, 2.0 Hz, 1H), 5.80 (d, J=0.9 Hz, 1H), 4.70-4.62 (m, 2H), 3.79 (d, J=11.2 Hz, 2H), 3.72 (s, 3H), 3.49 (d, J=10.6 Hz, 2H), 2.65-2.60 (m, 3H), 2.45 (s, 3H), 2.40 (s, 3H), 2.38-2.35 (m, 1H), 1.63 (d, J=15.1 Hz, 2H).

### Example 120: N-(5-fluoro-1-methyl-1H-pyrazol-3-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine

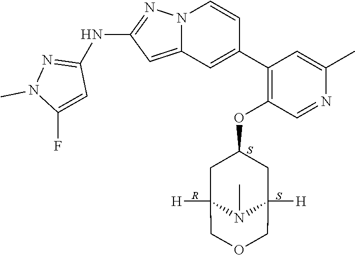

The title compound (13.1 mg, 11%) was prepared as described in Example 116 steps A-C where ((3-bromo-5-fluoro-1-methyl-1H-pyrazole)) was used instead of 4-bromo-1-(difluoromethyl)-1H-pyrazole in step A. MS (ESI): mass calcd. for C₂₅H₂₈FN₇O₂, 477.2. m/z found, 478.3 [M+H]⁺. ¹H NMR (500 MHz, DMSO-d₆) δ 9.16 (s, 1H), 8.41 (dt, J=7.1, 0.9 Hz, 1H), 8.18 (s, 1H), 8.04 (dd, J=2.0, 0.9 Hz, 1H), 7.36 (s, 1H), 7.10 (dd, J=7.2, 2.0 Hz, 1H), 6.32 (d, J=0.9 Hz, 1H), 5.83 (d, J=5.5 Hz, 1H), 4.70-4.61 (m, 1H), 3.79 (dd, J=10.8, 2.5 Hz, 2H), 3.58 (d, J=1.0 Hz, 3H), 3.52-3.46 (m, 2H), 2.66-2.59 (m, 2H), 2.45 (s, 3H), 2.43-2.34 (m, 5H), 1.63 (d, J=14.9 Hz, 2H).

### Example 121: 1-(4-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-1H-pyrazol-1-yl)-2-methylpropan-2-ol

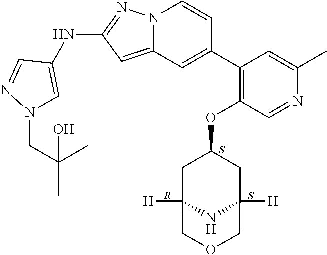

The title compound (18.7 mg, 15%) was prepared as described in Example 116 steps A-B where (1-(4-bromo-1H-pyrazol-1-yl)-2-methylpropan-2-01)) was used instead of 4-bromo-1-(difluoromethyl)-1H-pyrazole in step A. MS (ESI): mass calcd. for C₂₇H₃₃N₇O₃, 503.3. m/z found, 504.3 [M+H]⁺. ¹H NMR (500 MHz, DMSO-d₆) δ 8.43-8.37 (m, 2H), 8.27 (s, 1H), 7.80 (dd, J=2.0, 0.9 Hz, 1H), 7.76 (d, J=0.8 Hz, 1H), 7.40 (d, J=0.8 Hz, 1H), 7.34 (s, 1H), 6.94 (dd, J=7.2, 2.0 Hz, 1H), 5.88 (d, J=0.9 Hz, 1H), 4.67 (s, 1H), 4.63 (p, J=6.5 Hz, 1H), 3.96 (s, 2H), 3.55 (dd, J=10.6, 2.4 Hz, 2H), 3.52-3.47 (m, 2H), 2.92 (d, J=8.6 Hz, 2H), 2.45 (s, 3H), 2.28 (dt, J=14.3, 7.5 Hz, 2H), 1.64 (ddd, J=13.7, 6.7, 2.6 Hz, 2H), 1.07 (s, 6H).

### Example 122: 1-(4-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-1-(2-hydroxy-2-methylpropyl)-1H-pyrazol-3-yl)-2,2,2-trifluoroethan-1-one

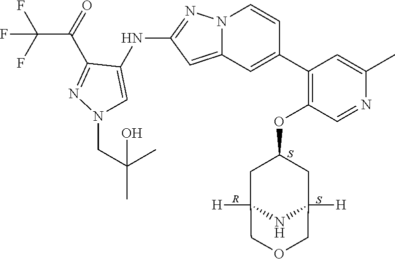

The title compound (12.7 mg, 11%) was isolated as an impurity in step B during the synthesis of Example 121. MS (ESI): mass calcd. for C₂₉H₃₂F₃N₇O₄, 599.2. m/z found, 600.3 [M+H]⁺. ¹H NMR (500 MHz, DMSO-d₆) δ 9.37 (s, 1H), 8.91 (dd, J=7.0, 0.8 Hz, 1H), 8.43 (s, 1H), 8.08 (d, J=0.7 Hz, 1H), 8.01-7.95 (m, 1H), 7.80 (d, J=0.7 Hz, 1H), 7.58 (dd, J=7.0, 1.9 Hz, 1H), 7.36 (s, 1H), 4.78 (h, J=7.1, 6.6 Hz, 1H), 4.70 (s, 1H), 4.00 (s, 2H), 3.46 (dd, J=10.7, 2.3 Hz, 2H), 3.38-3.34 (m, 2H), 2.94 (d, J=9.5 Hz, 2H), 2.48 (s, 3H), 2.32-2.23 (m, 2H), 1.57 (ddd, J=13.3, 8.1, 2.9 Hz, 2H), 1.08 (s, 6H).

### Example 123: 7-endo-5-[2-methyl-5-[[(1S,5R)-3-oxa-9-azabicyclo[3.3.1]nonan-7-yl]oxy]-4-pyridyl]-N-(1-methylpyrazol-4-yl)pyrazolo[1,5-a]pyridin-2-amine

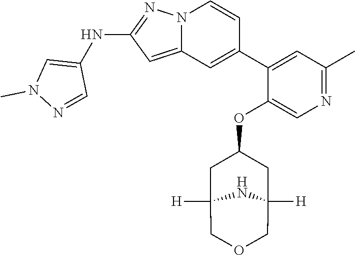

The title compound (11 mg, 12%) was prepared as described in Example 116 steps A-B where 4-bromo-1-methyl-1H-pyrazole was used instead of 4-bromo-1-(difluoromethyl)-1H-pyrazole in step A. MS (ESI): mass calcd. for C₂₄H₂₇N₇O₂, 445.2. m/z found, 446.1 [M+H]⁺. ¹H NMR (500 MHz, DMSO-d₆) δ 8.44-8.37 (m, 2H), 8.27 (s, 1H), 7.80 (dd, J=2.0, 0.9 Hz, 1H), 7.75 (d, J=0.8 Hz, 1H), 7.36 (d, J=0.8 Hz, 1H), 7.33 (s, 1H), 6.94 (dd, J=7.2, 2.0 Hz, 1H), 5.88 (d, J=0.8 Hz, 1H), 4.68-4.58 (m, 1H), 3.80 (s, 3H), 3.55 (d, J=10.4 Hz, 2H), 3.49 (d, J==10.6 Hz, 2H), 2.92 (s, 2H), 2.45 (s, 3H), 2.28 (dt, J=14.3, 7.5 Hz, 2H), 1.64 (dd, J=13.7, 6.6 Hz, 2H).

### Example 124: 7-endo-N-(1-methyl-1H-pyrazol-4-yl)-5-(2-methyl-5-4(1R,5S,7s)-9-methyl-3-oxa-9-azabicyclo[3.3.1]nonan-7-yl)oxy)pyridin-4-yl)pyrazolo[1,5-a]pyridin-2-amine

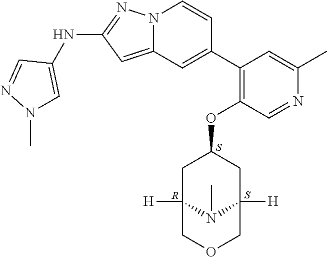

The title compound (6.1 mg, 6%) was prepared as described in Example 116 steps A-C where 4-bromo-1-methyl-1H-pyrazole was used instead of 4-bromo-1-(difluoromethyl)-1H-pyrazole in step A. MS (ESI): mass calcd. for C₂₅H₂₉N₇O₂, 459.2. m/z found, 460.2 [M+H]⁺. ¹H NMR (500 MHz, DMSO-d₆) δ 8.41-8.37 (m, 2H), 8.18 (s, 1H), 7.97 (dd, J=2.0, 0.9 Hz, 1H), 7.75 (d, J=0.8 Hz, 1H), 7.36 (t, J=0.9 Hz, 2H), 7.02 (dd, J=7.2, 2.0 Hz, 1H), 5.84 (d, J=0.8 Hz, 1H), 4.66 (dt, J=6.8, 3.6 Hz, 1H), 3.81-3.76 (m, 5H), 3.48 (d, J=10.8 Hz, 2H), 2.66-2.59 (m, 2H), 2.44 (s, 3H), 2.43-2.33 (m, 5H), 1.63 (d, J=14.9 Hz, 2H).

### Example 125: endo-5-(5-(((1R,5S,7s)-3-oxa-9-azabicyclo[3.3.1]nonan-7-yl)oxy)-2-methylpyridin-4-yl)-N-(1-methyl-1H-pyrazol-3-yl)pyrazolo[1,5-a]pyridin-2-amine

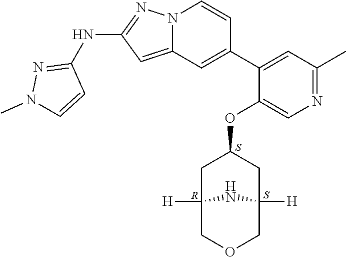

To a 40 mL vial was charged endo-7-hydroxy-3-oxa-9-azabicyclo[3.3.1]nonane hydrochloride (244 mg, 1.36 mmol) and DMA (2.1 mL). NaH (60% dispersion in mineral oil) (217 mg, 5.42 mmol) was added and the resulting suspension was heated to 80° C. for 10 min. 5-(5-fluoro-2-methylpyridin-4-yl)-N-(1-methyl-1H-pyrazol-3-yl)pyrazolo[1,5-a]pyridin-2-amine (175 mg, 0.543 mmol) was added and the temperature of the reaction was increased to 100° C. The reaction was stirred 2 hours at 100° C. The reaction was cooled to room temperature and was quenched by pouring it into stirring water (20 mL). This mixture was extracted with ethyl acetate then with 10% MeOH/ethyl acetate. The combined organic fractions were dried (Na₂SO₄) then concentrated to dryness. The product was purified over Basic ISCO (Gemini 5 uM NX-C18 110 angstrom LC column 150×21.2 mm; 10-100% AcN/20 mM aq NH₃ over 15 min; 40 mL/min) to afford the title compound (167 mg, 69%) as colorless solid. MS (ESI): mass calcd. for C₂₄H₂₇N₇O₂, 445.2. m/z found, 446.2 [M+H]⁺. ¹H NMR (400 MHz, DMSO-d₆) δ 9.03 (s, 1H), 8.42 (d, J=7.2 Hz, 1H), 8.28 (s, 1H), 7.86 (s, 1H), 7.49 (s, 1H), 7.34 (s, 1H), 6.99 (d, J=7.2 Hz, 1H), 6.38 (s, 1H), 6.07 (d, J=2.4 Hz, 1H), 4.71-4.57 (m, 1H), 3.74 (s, 3H), 3.53 (q, J=10.6 Hz, 4H), 2.93 (d, J=8.5 Hz, 2H), 2.46 (s, 3H), 2.37-2.18 (m, 3H), 1.71-1.56 (m, 2H).

### Example 126: 7-endo-N-(5-fluoropyridin-2-yl)-5-(2-methyl-5-4(1R,5S,7s)-9-methyl-3-oxa-9-azabicyclo[3.3.1]nonan-7-yl)oxy)pyridin-4-yl)pyrazolo[1,5-a]pyridin-2-amine

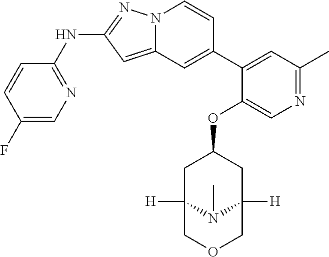 [SMILES: Cc1cc(-c2ccn3nc(Nc4ccc(F)cn4)cc3c2)c(O[C@H]2C[C@H]3COC[C@@H](C2)N3C)cn1]

The title compound (17.6 mg, 15%) was prepared as described in Example 116 steps A-C where 2-bromo-5-fluoropyridine was used instead of 4-bromo-1-(difluoromethyl)-1H-pyrazole; BrettPhos-Pd-G3, [(2-Di-cyclohexylphosphino-3,6-dimethoxy-2∝,4′,6′-triisopropyl-1,1′-biphenyl)-2-(2′-amino-1,1′-biphenyl)]palladium(II) methanesulfonate methanesulfonate and 2-(Dicyclohexylphosphino)3,6-dimethoxy-2′,4′,6′-triisopropyl-1,1′-biphenyl (10 mol %) was used instead of tert-BuBrettPhos-Pd-G3, [(2-Di-tert-butylphosphino-3,6-dimethoxy-2′,4′,6′-triisopropyl-1,1′-biphenyl)-2-(2′-amino-1,1′-biphenyl)]palladium(II) methanesulfonate in step A. MS (ESI): mass calcd. for C₂₆H₂₇FN₆O₂, 474.2. m/z found, 475.1 [M+H]⁺. ¹H NMR (500 MHz, DMSO-d₆) δ 9.82 (s, 1H), 8.48 (dt, J=7.2, 0.9 Hz, 1H), 8.23-8.18 (m, 2H), 8.12 (dd, J=2.0, 0.9 Hz, 1H), 7.61 (ddd, J=9.2, 8.4, 3.1 Hz, 1H), 7.40 (dd, J=9.2, 3.8 Hz, 1H), 7.38 (s, 1H), 7.16 (dd, J=7.2, 2.0 Hz, 1H), 6.72 (d, J=0.8 Hz, 1H), 4.68 (dt, J=6.6, 3.5 Hz, 1H), 3.79 (dd, J=10.8, 2.5 Hz, 2H), 3.49 (d, J=11.0 Hz, 2H), 2.63 (d, J=6.7 Hz, 2H), 2.45 (s, 3H), 2.40 (s, 5H), 1.64 (d, J=14.9 Hz, 2H).

### Example 127: 5-(5-(((1R,5S,7s)-3-oxa-9-azabicyclo[3.3.1]nonan-7-yl)oxy)-2-methylpyridin-4-yl)-N-(5-fluoropyridin-2-yl)pyrazolo[1,5-a]pyridin-2-amine

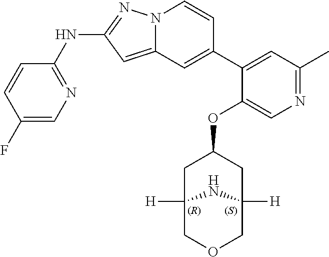

The title compound (17.6 mg, 15%) was prepared as described in Example 116 steps A-B where 2-bromo-5-fluoropyridine was used instead of 4-bromo-1-(difluoromethyl)-1H-pyrazole; BrettPhos Pd G3, [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate and 2-(Dicyclohexylphosphino)3,6-dimethoxy-2′,4′,6′-triisopropyl-1,1′-biphenyl (10 mol %) was used instead of tert-BuBrettPhos-Pd-G3, [(2-Di-tert-butylphosphino-3,6-dimethoxy-2′,4′,6′-triisopropyl-1,1′-biphenyl)-2-(2′-amino-1,1′-biphenyl)]palladium(II) methanesulfonate in step A. MS (ESI): mass calcd. for C₂₅H₂₅FN₆O₂, 460.2. m/z found, 461.1 [M+H]⁺. ¹H NMR (500 MHz, DMSO-d₆) δ 9.83 (s, 1H), 8.51 (dt, J=7.3, 0.9 Hz, 1H), 8.29 (s, 1H), 8.20 (d, J=3.1 Hz, 1H), 7.95 (dd, J=2.1, 0.9 Hz, 1H), 7.61 (ddd, J=9.1, 8.3, 3.1 Hz, 1H), 7.39 (dd, J=9.2, 3.8 Hz, 1H), 7.35 (s, 1H), 7.07 (dd, J=7.2, 2.0 Hz, 1H), 6.75 (d, J=0.9 Hz, 1H), 4.66 (q, J=6.4 Hz, 1H), 3.56 (dd, J=10.7, 2.3 Hz, 2H), 3.50 (d, J=10.5 Hz, 2H), 2.93 (d, J=8.6 Hz, 2H), 2.46 (s, 3H), 2.29 (dt, J=14.4, 7.6 Hz, 2H), 1.65 (ddd, J=13.7, 6.7, 2.6 Hz, 2H).

### Example 128: 5-[2-methyl-5-[[(2S)-morpholin-2-yl]methoxy]-4-pyridyl]-N-(1-methylpyrazol-4-yl)pyrazolo[1,5-a]pyridin-2-amine

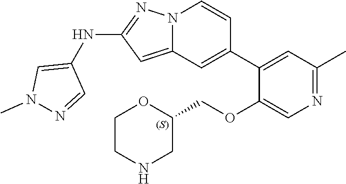

The title compound (3.2 mg, 4.8%) was prepared as described in Example 116 steps A-B where 4-bromo-1-methyl-1H-pyrazole was used instead of 4-bromo-1-(difluoromethyl)-1H-pyrazole, Intermediate 13 was used in place of Intermediate 9 in step A and HCl/1,4-dioxane was used instead of trifluoracetic acid in step B. MS (ESI): mass calcd. for C₂₂H₂₅N₇O₂, 419.2. m/z found, 420.40 [M+H]⁺. ¹H NMR (400 MHz, DMSO-d₆) δ 8.48-8.43 (m, 2H), 8.30 (s, 1H), 7.77 (s, 1H), 7.67 (d, J=1.2 Hz, 1H), 7.37 (s, 1H), 7.34 (s, 1H), 6.87 (dd, J=2.0, 7.2 Hz, 1H), 5.91 (s, 1H), 4.08 (d, J=4.8 Hz, 2H), 3.80 (s, 3H), 3.78-3.73 (m, 1H), 3.70-3.63 (m, 1H), 3.48-3.43 (m, 1H), 2.83-2.76 (m, 1H), 2.66-2.57 (m, 2H), 2.55-2.53 (m, 1H), 2.45 (s, 3H).

### Example 129: 5-[2-methyl-5-[[(2S)-morpholin-2-yl]methoxy]-4-pyridyl]-N-(1-methylpyrazol-3-yl)pyrazolo[1,5-a]pyridin-2-amine

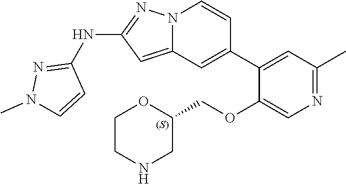

The title compound (14.2 mg, 11%) was prepared as described in Example 116 steps A-B where 3-bromo-1-methyl-1H-pyrazole was used instead of 4-bromo-1-(difluoromethyl)-1H-pyrazole, Intermediate 13 instead of

Intermediate 9, t-BuXphos-Pd-G3/t-BuXPhos in place of t-BuBrettPhos-Pd-G3, sodium tert-butoxide in place of cesium carbonate, and THF in place of 1,4-dioxane in step A and HCl/1,4-dioxane was used instead of trifluoracetic acid in step B. MS (ESI): mass calcd. for C₂₂H₂₅N₇O₂, 419.2. m/z found, 420.2 [M+H]⁺. ¹H NMR (400 MHz, DMSO-d₆) δ 9.08 (s, 1H), 8.45 (d, J=7.2 Hz, 1H), 8.31 (s, 1H), 7.73 (d, J=1.2 Hz, 1H), 7.49 (d, J=2.0 Hz, 1H), 7.34 (s, 1H), 6.92 (dd, J=2.0, 7.2 Hz, 1H), 6.42 (s, 1H), 6.07 (d, J=2.0 Hz, 1H), 4.11 (d, J=4.4 Hz, 2H), 3.84-3.78 (m, 1H), 3.78-3.75 (m, 1H), 3.74 (s, 3H), 3.54-3.50 (m, 1H), 2.95-2.83 (m, 1H), 2.80-2.73 (m, 1H), 2.72-2.63 (m, 1H), 2.62-2.54 (m, 1H), 2.46 (s, 3H).

### Example 130: 5-[2-methyl-5-[[(2S)-morpholin-2-yl]methoxy]-4-pyridyl]-N-(2-pyridyl)pyrazolo[1,5-a]pyridin-2-amine

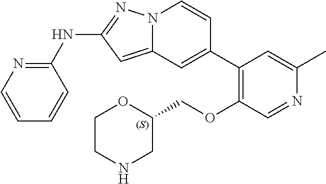

The title compound (47 mg, 17%) was prepared as described in Example 116 steps A-B where 2-bromopyridine was used instead of 4-bromo-1-(difluoromethyl)-1H-pyrazole and Intermediate 13 was used instead of

Intermediate 9 in step A. MS (ESI): mass calcd. for C₂₃H₂₄N₆O₂, 416.2. m/z found, 417.1 [M+H]⁺. ¹H NMR (400 MHz, DMSO-d₆) δ 9.79 (s, 1H), 8.54 (d, J=7.2 Hz, 1H), 8.32 (s, 1H), 8.22 (dd, J=1.2, 4.8 Hz, 1H), 7.82 (d, J=1.2 Hz, 1H), 7.67-7.56 (m, 1H), 7.37-7.31 (m, 2H), 7.00 (dd, J=2.0, 7.2 Hz, 1H), 6.86 (s, 1H), 6.80 (dd, J=5.6, 6.8 Hz, 1H), 4.19-4.01 (m, 2H), 3.82-3.71 (m, 1H), 3.71-3.62 (m, 1H), 3.52-3.39 (m, 1H), 2.81 (dd, J=1.6, 12.0 Hz, 1H), 2.71-2.57 (m, 2H), 2.55-2.52 (m, 1H), 2.46 (s, 3H).

### Example 131: 5-[2-methyl-5-[[(2S)-morpholin-2-yl]methoxy]-4-pyridyl]-N-(3-pyridyl)pyrazolo[1,5-a]pyridin-2-amine

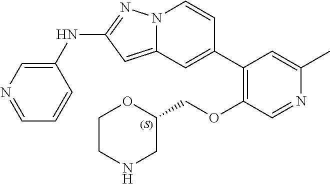

The title compound (31 mg, 14%) was prepared as described in Example 116 steps A-B where 3-bromopyridine was used instead of 4-bromo-1-(difluoromethyl)-1H-pyrazole and Intermediate 13 was used instead of

Intermediate 9 in step A. MS (ESI): mass calcd. for C₂₃H₂₄N₆O₂, 416.2. m/z found, 417.2 [M+H]⁺. ¹H NMR (400 MHz, DMSO-d₆) δ 9.24 (s, 1H), 8.72 (d, J=2.4 Hz, 1H), 8.59 (d, J=7.2 Hz, 1H), 8.32 (s, 1H), 8.09-7.99 (m, 2H), 7.79 (d, J=1.2 Hz, 1H), 7.36 (s, 1H), 7.32-7.23 (m, 1H), 6.98 (dd, J=2.0, 7.2 Hz, 1H), 6.15 (s, 1H), 4.09 (d, J=4.8 Hz, 2H), 3.82-3.73 (m, 1H), 3.71-3.63 (m, 1H), 3.52-3.40 (m, 1H), 2.81 (d, J=12.0 Hz, 1H), 2.71-2.57 (m, 2H), 2.56-2.52 (m, 1H), 2.46 (s, 3H).

### Example 132: 5-[2-methyl-5-[[(2S)-morpholin-2-yl]methoxy]-4-pyridyl]-N-pyrimidin-4-yl-pyrazolo[1,5-a]pyridin-2-amine

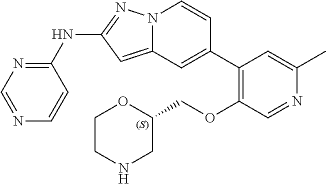

The title compound (32 mg, 8%) was prepared as described in Example 116 steps A-B where 4-chloropyrimidine hydrogen chloride was used instead of 4-bromo-1-(difluoromethyl)-1H-pyrazole and Intermediate 13 was used instead of

Intermediate 9 in step A. MS (ESI): mass calcd. for C₂₂H₂₃N₇O₂, 417.2. m/z found, 418.1 [M+H]⁺. ¹H NMR (400 MHz, CDCl₃) δ 8.80 (d, J=0.8 Hz, 1H), 8.45 (d, J=6.0 Hz, 1H), 8.37-8.33 (m, 1H), 8.27 (s, 1H), 7.71-7.67 (m, 1H), 7.53 (s, 1H), 7.41 (d, J=6.0 Hz, 1H), 7.18 (s, 1H), 7.00 (dd, J=1.6, 7.2 Hz, 1H), 6.60 (s, 1H), 4.14-4.08 (m, 1H), 4.06-4.00 (m, 1H), 3.94-3.88 (m, 1H), 3.86-3.78 (m, 1H), 3.69-3.59 (m, 1H), 2.98-2.90 (m, 1H), 2.89-2.79 (m, 2H), 2.77-2.68 (m, 1H), 2.56 (s, 3H).

### Example 133: N-(1-isopropylpyrazol-4-yl)-5-[2-methyl-5-[[(2S)-morpholin-2-yl]methoxy]-4-pyridyl]pyrazolo[1,5-a]pyridin-2-amine

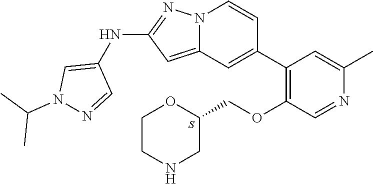

The title compound (24 mg, 10%) was prepared as described in Example 116 steps A-B where 4-iodo-1-isopropyl-1H-pyrazole was used instead of 4-bromo-1-(difluoromethyl)-1H-pyrazole and Intermediate 13 was used instead of

Intermediate 9 in step A. MS (ESI): mass calcd. for C₂₄H₂₉N₇O₂, 447.24. m/z found, 448.3 [M+H]⁺. ¹H NMR (400 MHz, CDCl₃) δ 8.27-8.21 (m, 2H), 7.63 (s, 1H), 7.51-7.46 (m, 2H), 7.16 (s, 1H), 6.82 (dd, J=1.6, 6.8 Hz, 1H), 5.90-5.82 (m, 2H), 4.54-4.43 (m, 1H), 4.12-4.04 (m, 1H), 4.03-3.96 (m, 1H), 3.94-3.87 (m, 1H), 3.86-3.79 (m, 1H), 3.71-3.61 (m, 1H), 2.99-2.91 (m, 1H), 2.89-2.81 (m, 2H), 2.77-2.67 (m, 1H), 2.54 (s, 3H), 1.53 (d, J=6.8 Hz, 6H).

### Example 134: 5-[2-methyl-5-[[(2S)-morpholin-2-yl]methoxy]-4-pyridyl]-N-pyridazin-3-yl-pyrazolo[1,5-a]pyridin-2-amine

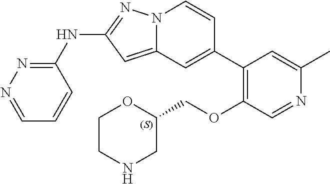

The title compound (50 mg, 41%) was prepared as described in Example 116 steps A-B where 3-bromopyridazine was used instead of 4-bromo-1-(difluoromethyl)-1H-pyrazole and Intermediate 13 was used instead of

Intermediate 9 in step A. MS (ESI): mass calcd. for C₂₂H₂₃N₇O₂, 417.2. m/z found, 418.1 [M+H]⁺. ¹H NMR (400 MHz, CDCl₃) δ 8.99 (s, 1H), 8.80-8.75 (m, 1H), 8.37 (d, J=7.2 Hz, 1H), 8.26 (s, 1H), 8.11 (dd, J=0.8, 8.8 Hz, 1H), 7.68 (d, J=0.8 Hz, 1H), 7.42 (dd, J=4.4, 8.8 Hz, 1H), 7.19 (s, 1H), 6.96 (dd, J=2.0, 7.2 Hz, 1H), 6.65 (s, 1H), 4.14-4.07 (m, 1H), 4.06-3.99 (m, 1H), 3.95-3.88 (m, 1H), 3.86-3.78 (m, 1H), 3.68-3.60 (m, 1H), 2.99-2.92 (m, 1H), 2.91-2.78 (m, 2H), 2.77-2.68 (m, 1H), 2.56 (s, 3H).

### Example 135: N-(1-isopropylpyrazol-3-yl)-5-[2-methyl-5-[[(2S)-morpholin-2-yl]methoxy]-4-pyridyl]pyrazolo[1,5-a]pyridin-2-amine

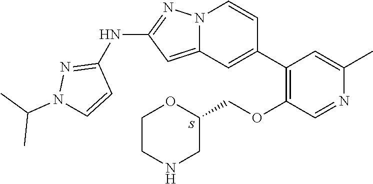

The title compound (49 mg, 38%) was prepared as described in Example 116 steps A-B where 3-iodo-1-isopropyl-1H-pyrazole was used instead of 4-bromo-1-(difluoromethyl)-1H-pyrazole and Intermediate 13 was used instead of

Intermediate 9 in step A. MS (ESI): mass calcd. for C₂₄H₂₉N₇O₂, 447.2. m/z found, 448.3 [M+H]⁺. ¹H NMR (400 MHz, CD₃CN) δ 8.32 (d, J=7.2 Hz, 1H), 8.25 (s, 1H), 7.75-7.67 (m, 1H), 7.50 (s, 1H), 7.42 (d, J=2.4 Hz, 1H), 7.27 (s, 1H), 6.91 (dd, J=2.0, 7.2 Hz, 1H), 6.49 (s, 1H), 6.08 (d, J=2.4 Hz, 1H), 4.43-4.32 (m, 1H), 4.11-4.00 (m, 2H), 3.85-3.76 (m, 1H), 3.75-3.67 (m, 1H), 3.57-3.45 (m, 1H), 2.85 (dd, J=2.0, 12.0 Hz, 1H), 2.73-2.68 (m, 2H), 2.64-2.55 (m, 1H), 2.47 (s, 3H), 1.45 (d, J=6.8 Hz, 6H).

### Example 136: endo-N-(5-(5-(((1R,5S,7s)-3-oxa-9-azabicyclo[3.3.1]nonan-7-yl)oxy)-2-methylpyridin-4-yl)pyrazolo[1,5-a]pyridin-2-yl)cyclopropanecarboxamide

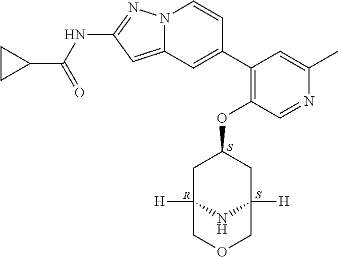

To a small vial containing endo-7-hydroxy-3-oxa-9-azabicyclo[3.3.1]nonane hydrochloride (255 mg, 1.42 mmol, 2.2 eq) dissolved in DMA (2.58 mL, 0.64 mmol, 0.25 M) was added sodium hydride (60 wt % dispersion in mineral oil, 258 mg, 6.45 mmol, 10 eq) portion-wisely while stirring at rt. The suspension was heated at 50° C. for 5 min, then N-(5-(5-fluoro-2-methylpyridin-4-yl)pyrazolo[1,5-a]pyridin-2-yl)cyclopropanecarboxamide (Intermediate 6, 200 mg, 0.64 mmol, 1.0 eq) was added. The mixture was stirred at 90° C. for 2 hours, then quenched with water. The aqueous layer was extracted several times with 10% MeOH/DCM, and the organic extracts were dried over sodium sulfate. The product was purified over FCC (10% 2M NH₃ in MeOH/DCM) to provide the title compound (282 mg, 100%). MS (ESI): mass calcd. for C₂₄H₂₇N₅O₃, 433.2. m/z found, 434.3 [M+H]⁺. ¹H NMR (400 MHz, DMSO-d₆) δ 11.07 (s, 1H), 8.57-8.51 (m, 1H), 8.28 (s, 1H), 8.08-8.05 (m, 1H), 7.37 (s, 1H), 7.17 (dd, J=7.3, 2.0 Hz, 1H), 6.85 (s, 1H), 4.76-4.54 (m, 1H), 3.61-3.54 (m, 2H), 3.50 (d, J=10.6 Hz, 2H), 2.96-2.88 (m, 2H), 2.45 (s, 3H), 2.32-2.24 (m, 2H), 1.97-1.88 (m, 1H), 1.71-1.61 (m, 2H), 0.87-0.77 (m, 4H).

### Example 137: N-[5-[2-methyl-5-[(3R)-1-methylpyrrolidin-3-yl]oxy-4-pyridyl]pyrazolo[1,5-a]pyridin-2-yl]cyclopropanecarboxamide

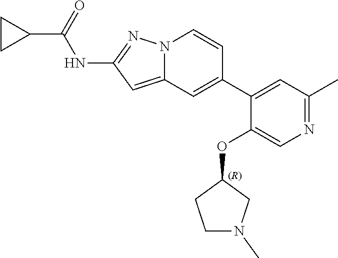

The title compound (133 mg, 71%) was prepared as described in Example 136 where (R)-(−)-1-methyl-3-pyrrolidinol was used instead of endo-7-hydroxy-3-oxa-9-azabicyclo[3.3.1]nonane hydrochloride. MS (ESI): mass calcd. for C₂₂H₂₅N₅O₂, 391.2. m/z found, 392.3 [M+H]⁺. ¹H NMR (400 MHz, DMSO-d₆) δ 11.08 (s, 1H), 8.60-8.52 (m, 1H), 8.24 (s, 1H), 7.86-7.79 (m, 1H), 7.35 (s, 1H), 7.05 (dd, J=7.3, 2.0 Hz, 1H), 6.88 (s, 1H), 5.06-4.97 (m, 1H), 2.82-2.71 (m, 1H), 2.67-2.58 (m, 2H), 2.45 (s, 3H), 2.40-2.26 (m, 2H), 2.24 (s, 3H), 1.98-1.89 (m, 1H), 1.84-1.71 (m, 1H), 0.88-0.77 (m, 4H).

### Example 138: N-(5-(2-methyl-5-(((1R,3r,5S)-8-methyl-8-azabicyclo[3.2.1]octan-3-yl)oxy)pyridin-4-yl)pyrazolo[1,5-a]pyridin-2-yl)cyclopropanecarboxamide

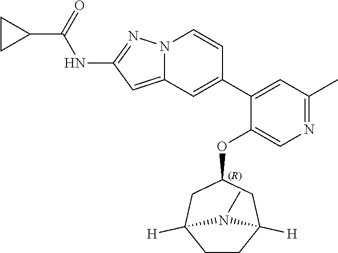

The title compound (81.2 mg, 39%) was prepared as described in Example 136 where tropine (CAS 120-29-6) was used instead of endo-7-hydroxy-3-oxa-9-azabicyclo[3.3.1]nonane hydrochloride. MS (ESI): mass calcd. for C₂₅H₂₉N₅O₂, 431.2. m/z found, 432.3 [M+H]⁺. ¹H NMR (400 MHz, DMSO-d₆) δ 11.09 (s, 1H), 10.26 (s, 1H), 8.63-8.54 (m, 1H), 8.33 (s, 1H), 7.84-7.72 (m, 1H), 7.34 (s, 1H), 7.33-7.04 (m, 2H), 6.99 (dd, J=7.2, 2.0 Hz, 1H), 6.91 (s, 1H), 4.89-4.81 (m, 1H), 3.74 (s, 2H), 2.58-2.55 (m, 2H), 2.46 (s, 3H), 2.17-2.08 (m, 2H), 1.98-1.85 (m, 3H), 1.84-1.74 (m, 2H), 0.86-0.79 (m, 4H).

### Example 139: N-[5-[2-methyl-5-[(3S)-1-methylpyrrolidin-3-yl]oxy-4-pyridyl]pyrazolo[1,5-a]pyridin-2-yl]cyclopropanecarboxamide

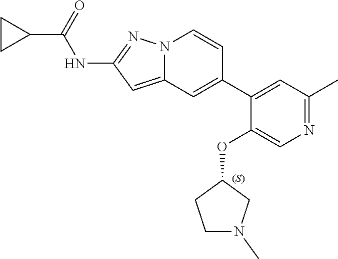

The title compound (140 mg, 74%) was prepared as described in Example 136 where (S)-(+)-1-methyl-3-pyrrolidinol was used instead of endo-7-hydroxy-3-oxa-9-azabicyclo[3.3.1]nonane hydrochloride. MS (ESI): mass calcd. for C₂₂H₂₅N₅O₂, 391.2. m/z found, 392.2 [M+H]⁺. ¹H NMR (400 MHz, DMSO-d₆) δ 11.08 (s, 1H), 8.63-8.46 (m, 1H), 8.24 (s, 1H), 7.87-7.74 (m, 1H), 7.35 (s, 1H), 7.05 (dd, J=7.3, 2.0 Hz, 1H), 6.94-6.79 (m, 1H), 5.07-4.92 (m, 1H), 2.77 (dd, J=10.5, 5.9 Hz, 1H), 2.68-2.57 (m, 2H), 2.45 (s, 3H), 2.38-2.25 (m, 2H), 2.23 (s, 3H), 1.96-1.87 (m, 1H), 1.85-1.73 (m, 1H), 0.89-0.75 (m, 4H).

### Example 140: N-(5-(2-methyl-5-(((1R,3r,5S)-8-methyl-8-azabicyclo[3.2.1]octan-3-yl)oxy)pyridin-4-yl)pyrazolo[1,5-a]pyridin-2-yl)cyclopropanecarboxamide

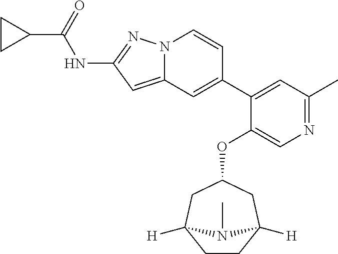 [SMILES: Cc1cc(-c2ccn3nc(NC(=O)C4CC4)cc3c2)c(O[C@@H]2C[C@H]3CC[C@@H](C2)N3C)cn1]

The title compound (176 mg, 84%) was prepared as described in Example 136 where pseudotropanol (CAS 135-97-7) was used instead of endo-7-hydroxy-3-oxa-9-azabicyclo[3.3.1]nonane hydrochloride. MS (ESI): mass calcd. for C₂₅H₂₉N₅O₂, 431.2. m/z found, 432.3 [M+H]⁺. ¹H NMR (400 MHz, DMSO-d₆) δ 11.07 (s, 1H), 8.61-8.51 (m, 1H), 8.38 (s, 1H), 7.79-7.71 (m, 1H), 7.33 (s, 1H), 7.01 (dd, J=7.2, 2.0 Hz, 1H), 6.88 (s, 1H), 4.62-4.48 (m, 1H), 3.11-3.04 (m, 2H), 2.45 (s, 3H), 2.15 (s, 3H), 1.97-1.83 (m, 5H), 1.62-1.50 (m, 4H), 0.88-0.78 (m, 4H).

### Example 141: N-[5-[5-(1-ethylazetidin-3-yl)oxy-2-methyl-4-pyridyl]pyrazolo[1,5-a]pyridin-2-yl]cyclopropanecarboxamide

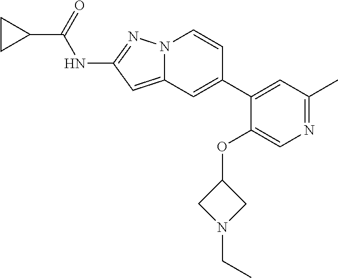 [SMILES: CCN1CC(Oc2cnc(C)cc2-c2ccn3nc(NC(=O)C4CC4)cc3c2)C1]

The title compound (70.7 mg, 37%) was prepared as described in Example 136 where 1-ethylazetidin-3-ol was used instead of endo-7-hydroxy-3-oxa-9-azabicyclo[3.3.1]nonane hydrochloride. MS (ESI): mass calcd. for C₂₂H₂₅N₅O₂, 391.2. m/z found, 392.2 [M+H]⁺. ¹H NMR (400 MHz, DMSO-d₆) δ 11.09 (s, 1H), 8.57 (dd, J=7.3, 1.0 Hz, 1H), 8.08 (s, 1H), 7.85-7.80 (m, 1H), 7.37 (s, 1H), 7.06 (dd, J=7.2, 2.0 Hz, 1H), 6.90 (s, 1H), 4.99-4.82 (m, 1H), 3.76-3.66 (m, 2H), 2.98-2.91 (m, 2H), 2.48-2.39 (m, 5H), 1.98-1.90 (m, 1H), 0.91-0.77 (m, 7H).

### Example 142: N-[5-[5-(1-isopropylazetidin-3-yl)oxy-2-methyl-4-pyridyl]pyrazolo[1,5-a]pyridin-2-yl]cyclopropanecarboxamide

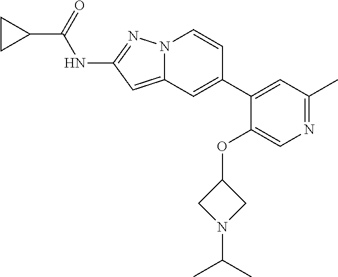 [SMILES: Cc1cc(-c2ccn3nc(NC(=O)C4CC4)cc3c2)c(OC2CN(C(C)C)C2)cn1]

The title compound (147 mg, 75%) was prepared as described in Example 136 where 1-isopropylazetidin-3-ol was used instead of endo-7-hydroxy-3-oxa-9-azabicyclo[3.3.1]nonane hydrochloride. MS (ESI): mass calcd. for C₂₃H₂₇N₅O₂, 405.2. m/z found, 406.2 [M+H]⁺. ¹H NMR (500 MHz, DMSO-d₆) δ 11.09 (s, 1H), 8.62-8.51 (m, 1H), 8.09 (s, 1H), 7.88-7.78 (m, 1H), 7.36 (s, 1H), 7.05 (dd, J=7.2, 2.0 Hz, 1H), 6.91 (s, 1H), 4.91-4.79 (m, 1H), 3.75-3.61 (m, 2H), 2.98-2.89 (m, 2H), 2.46 (s, 3H), 2.34-2.26 (m, 1H), 1.97-1.89 (m, 1H), 0.85 (d, J=6.2 Hz, 6H), 0.84-0.79 (m, 4H).

### Example 143: N-[5-[2-methyl-5-[[(2S)-morpholin-2-yl]methoxy]-4-pyridyl]pyrazolo[1,5-a]pyridin-2-yl]cyclopropanecarboxamide

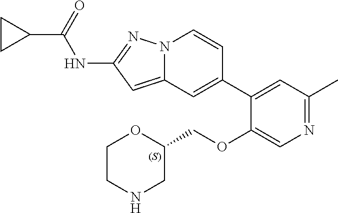

The title compound (73.0 mg, 37%) was prepared as described in Example 136 where [(2S)-morpholin-2-yl]methanol hydrochloride was used instead of endo-7-hydroxy-3-oxa-9-azabicyclo[3.3.1]nonane hydrochloride. MS (ESI): mass calcd. for C₂₂H₂₅N₅O₃, 407.2. m/z found, 408.2 [M+H]⁺. ¹H NMR (400 MHz, DMSO-d₆) δ 11.08 (s, 1H), 8.63-8.50 (m, 1H), 8.32 (s, 1H), 7.96-7.85 (m, 1H), 7.36 (s, 1H), 7.09 (dd, J=7.3, 2.0 Hz, 1H), 6.88 (s, 1H), 4.09 (d, J=4.9 Hz, 2H), 3.79-3.71 (m, 1H), 3.71-3.62 (m, 1H), 3.48-3.41 (m, 1H), 2.84-2.77 (m, 1H), 2.69-2.56 (m, 2H), 2.54-2.48 (m, 1H), 2.46 (s, 3H), 1.98-1.88 (m, 1H), 0.88-0.75 (m, 4H).

### Example 144: N-[5-[5-[(3-aminooxetan-3-yl)methoxy]-2-methyl-4-pyridyl]pyrazolo[1,5-a]pyridin-2-yl]cyclopropanecarboxamide

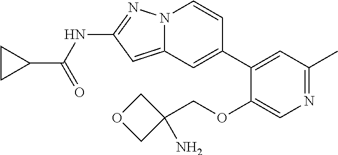 [SMILES: Cc1cc(-c2ccn3nc(NC(=O)C4CC4)cc3c2)c(OCC2(N)COC2)cn1]

The title compound (30 mg, 24%) was prepared as described in Example 136 where (3-aminooxetan-3-yl)methanol was used instead of endo-7-hydroxy-3-oxa-9-azabicyclo[3.3.1]nonane hydrochloride. MS (ESI): mass calcd. for C₂₁H₂₃N₅O₃, 393.2. m/z found, 394.20 [M+H]⁺. ¹H NMR (400 MHz, DMSO-d₆) δ 11.08 (s, 1H), 8.54 (dt, J=7.3, 0.9 Hz, 1H), 8.39 (s, 1H), 7.91 (dd, J=2.1, 0.9 Hz, 1H), 7.39 (s, 1H), 7.09 (dd, J=7.2, 2.0 Hz, 1H), 6.86 (s, 1H), 4.44 (d, J=6.1 Hz, 2H), 4.31 (d, J=6.0 Hz, 2H), 4.19 (s, 2H), 2.47 (s, 3H), 2.22 (s, 2H), 1.93 (s, 1H), 0.90-0.73 (m, 4H).

### Example 145: N-[5-[5-[(1-amino-3,3-difluoro-cyclobutyl)methoxy]-2-methyl-4-pyridyl]pyrazolo[1,5-a]pyridin-2-yl]cyclopropanecarboxamide

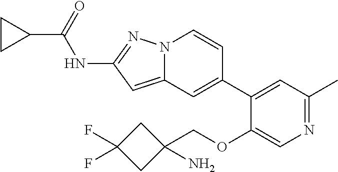 [SMILES: Cc1cc(-c2ccn3nc(NC(=O)C4CC4)cc3c2)c(OCC2(N)CC(F)(F)C2)cn1]

The title compound (45 mg, 33%) was prepared as described in Example 136 where (1-amino-3,3-difluorocyclobutyl)methanol was used instead of endo-7-hydroxy-3-oxa-9-azabicyclo[3.3.1]nonane hydrochloride. MS (ESI): mass calcd. for C₂₂H₂₃F₂N₅O₂, 427.2. m/z found, 428.2 [M+H]⁺. ¹H NMR (400 MHz, DMSO-d₆) δ 11.08 (s, 1H), 8.55 (dt, J=7.3, 0.9 Hz, 1H), 8.35 (s, 1H), 7.86 (dd, J=2.0, 0.9 Hz, 1H), 7.37 (s, 1H), 7.07 (dd, J=7.2, 2.0 Hz, 1H), 6.96-6.82 (m, 1H), 4.02 (s, 2H), 2.69 (td, J=13.9, 10.8 Hz, 2H), 2.47 (s, 3H), 2.37 (td, J=14.3, 11.3 Hz, 2H), 2.05 (s, 2H), 1.93 (td, J=7.2, 3.7 Hz, 1H), 0.82 (tt, J=7.9, 3.0 Hz, 4H).

### Example 146: trans-N-[5-[5-(3-methoxycyclobutoxy)-2-methyl-4-pyridyl]pyrazolo[1,5-a]pyridin-2-yl]cyclopropanecarboxamide

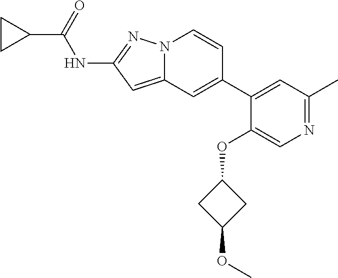 [SMILES: CO[C@H]1C[C@H](Oc2cnc(C)cc2-c2ccn3nc(NC(=O)C4CC4)cc3c2)C1]

The title compound (30 mg, 24%) was prepared as described in Example 136 where trans-3-methoxycyclobutan-1-ol was used instead of endo-7-hydroxy-3-oxa-9-azabicyclo[3.3.1]nonane hydrochloride. MS (ESI): mass calcd. for C₂₂H₂₄N₄O₃, 392.2. m/z found, 393.1 [M+H]⁺. ¹H NMR (400 MHz, DMSO-d₆) δ 11.08 (s, 1H), 8.56 (dq, J=7.2, 1.0 Hz, 1H), 8.08 (s, 1H), 7.81 (ddd, J=4.2, 2.0, 1.0 Hz, 1H), 7.35 (d, J=2.2 Hz, 1H), 7.05 (dt, J=7.2, 2.3 Hz, 1H), 6.90 (d, J=0.9 Hz, 1H), 4.96 (tt, J=7.0, 4.3 Hz, 1H), 4.04 (tt, J=6.9, 4.3 Hz, 1H), 3.14 (d, J=3.8 Hz, 3H), 2.45 (s, 3H), 2.40 (td, J=6.8, 4.0 Hz, 2H), 2.29 (ddd, J=13.3, 7.0, 4.1 Hz, 2H), 1.98-1.88 (m, 1H), 0.89-0.74 (m, 4H).

### Example 147: cis-N-[5-[5-(3-methoxycyclobutoxy)-2-methyl-4-pyridyl]pyrazolo[1,5-a]pyridin-2-yl]cyclopropanecarboxamide

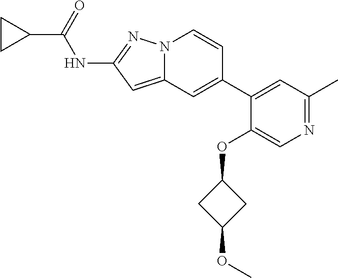 [SMILES: CO[C@H]1C[C@@H](Oc2cnc(C)cc2-c2ccn3nc(NC(=O)C4CC4)cc3c2)C1]

The title compound (57 mg, 45%) was prepared as described in Example 136 where cis-3-methoxycyclobutan-1-ol was used instead of endo-7-hydroxy-3-oxa-9-azabicyclo[3.3.1]nonane hydrochloride. MS (ESI): mass calcd. for C₂₂H₂₄N₄O₃, 392.2. m/z found, 393.1 [M+H]⁺. ¹H NMR (400 MHz, DMSO-d₆) δ 11.08 (s, 1H), 8.57 (dd, J=7.2, 1.0 Hz, 1H), 8.15 (s, 1H), 7.81 (dd, J=2.1, 0.9 Hz, 1H), 7.35 (s, 1H), 7.04 (dd, J=7.2, 2.0 Hz, 1H), 6.90 (s, 1H), 4.51 (p, J=6.9 Hz, 1H), 3.61 (p, J=6.8 Hz, 1H), 3.14 (s, 3H), 2.85 (dtd, J=9.5, 6.6, 3.1 Hz, 2H), 2.45 (s, 3H), 1.92 (dddd, J=17.0, 10.0, 7.4, 4.1 Hz, 3H), 0.82 (tt, J=7.9, 3.0 Hz, 4H).

### Example 148: cis-N-[5-[5-[(4-hydroxycyclohexyl)methoxy]-2-methyl-4-pyridyl]pyrazolo[1,5-a]pyridin-2-yl]cyclopropanecarboxamide

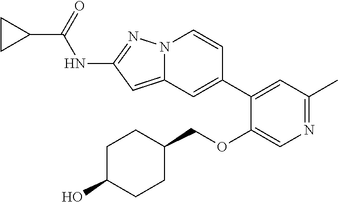 [SMILES: Cc1cc(-c2ccn3nc(NC(=O)C4CC4)cc3c2)c(OC[C@H]2CC[C@@H](O)CC2)cn1]

The title compound (26 mg, 19%) was prepared as described in Example 136 where cis-4-(hydroxymethyl)cyclohexan-1-ol was used instead of endo-7-hydroxy-3-oxa-9-azabicyclo[3.3.1]nonane hydrochloride. MS (ESI): mass calcd. for C₂₄H₂₈N₄O₃, 420.2. m/z found, 421.2 [M+H]⁺. ¹H NMR (400 MHz, DMSO-d₆) δ 11.07 (s, 1H), 8.55 (dt, J=7.3, 1.0 Hz, 1H), 8.33 (s, 1H), 7.80 (dd, J=2.0, 0.9 Hz, 1H), 7.34 (s, 1H), 7.04 (dd, J=7.2, 2.0 Hz, 1H), 6.87 (s, 1H), 4.26 (s, 1H), 3.95 (d, J=6.6 Hz, 2H), 3.75 (s, 1H), 2.46 (s, 3H), 1.92 (dd, J=7.6, 4.9 Hz, 1H), 1.75 (s, 1H), 1.62-1.52 (m, 2H), 1.45 (qd, J=12.9, 12.1, 6.5 Hz, 6H), 0.82 (tt, J=7.8, 2.9 Hz, 4H).

### Example 149: N-[5-[5-[(2S)-2-amino-3,3-dimethyl-butoxy]-2-methyl-4-pyridyl]pyrazolo[1,5-a]pyridin-2-yl]cyclopropanecarboxamide

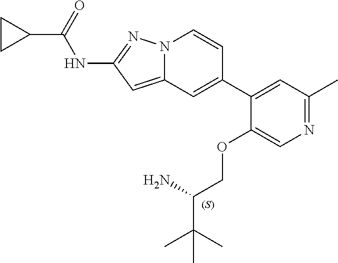

The title compound (47 mg, 36%) was prepared as described in Example 136 where L-tert-leucinol was used instead of endo-7-hydroxy-3-oxa-9-azabicyclo[3.3.1]nonane hydrochloride. MS (ESI): mass calcd. for C₂₃H₂₉N₅O₂, 407.2. m/z found, 408.3 [M+H]⁺. ¹H NMR (400 MHz, DMSO-d₆) δ 11.08 (s, 1H), 8.56 (dt, J=7.1, 1.0 Hz, 1H), 8.37 (s, 1H), 7.85 (dd, J=2.0, 0.9 Hz, 1H), 7.33 (s, 1H), 7.07 (dd, J=7.2, 2.0 Hz, 1H), 6.89 (s, 1H), 4.23 (dd, J=9.3, 3.2 Hz, 1H), 3.80 (t, J=8.9 Hz, 1H), 2.69 (dd, J=8.4, 3.2 Hz, 1H), 2.46 (s, 3H), 1.92 (q, J=7.9, 6.4 Hz, 1H), 0.87 (s, 9H), 0.85-0.77 (m, 4H).

### Example 150: N-[5-[5-[(2R)-2-amino-3,3-dimethyl-butoxy]-2-methyl-4-pyridyl]pyrazolo[1,5-a]pyridin-2-yl]cyclopropanecarboxamide

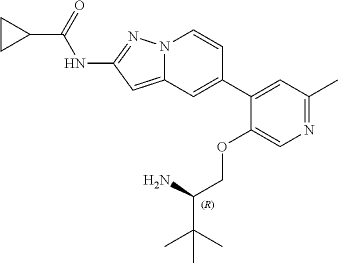

The title compound (14 mg, 8%) was prepared as described in Example 136 where (R)-2-amino-3,3-dimethylbutan-1-ol was used instead of endo-7-hydroxy-3-oxa-9-azabicyclo[3.3.1]nonane hydrochloride. MS (ESI): mass calcd. for C₂₃H₂₉N₅O₂, 407.2. m/z found, 408.3 [M+H]⁺. ¹H NMR (400 MHz, DMSO-d₆) δ 11.10 (s, 1H), 8.56 (d, J=7.2 Hz, 1H), 8.50 (s, 1H), 7.96-7.84 (m, 4H), 7.50 (s, 1H), 7.09 (dd, J=7.2, 2.0 Hz, 1H), 6.94 (s, 1H), 4.36 (dd, J=10.7, 3.2 Hz, 1H), 4.23 (dd, J=10.7, 8.1 Hz, 1H), 3.28 (s, 3H), 2.53 (s, 3H), 1.98-1.89 (m, 1H), 1.00 (s, 9H), 0.87-0.77 (m, 4H).

### Example 151: trans-N-(5-(5-(((1r,4r)-4-hydroxy-4-methylcyclohexyl)oxy)-2-methylpyridin-4-yl)pyrazolo[1,5-a]pyridin-2-yl)cyclopropanecarboxamide

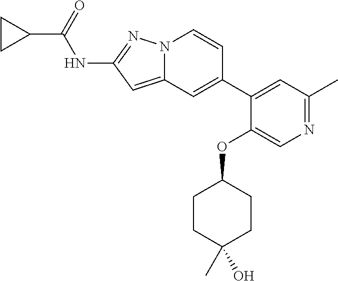 [SMILES: Cc1cc(-c2ccn3nc(NC(=O)C4CC4)cc3c2)c(O[C@H]2CC[C@](C)(O)CC2)cn1]

The title compound (110 mg, 31%) was prepared as described in Example 136 where trans-1-methylcyclohexane-1,4-diol was used instead of endo-7-hydroxy-3-oxa-9-azabicyclo[3.3.1]nonane hydrochloride. MS (ESI): mass calcd. for C₂₄H₂₈N₄O₃, 420.2. m/z found, 421.3 [M+H]⁺. ¹H NMR (400 MHz, DMSO-d₆) δ 11.07 (s, 1H), 8.58 (d, J=7.2 Hz, 1H), 8.35 (s, 1H), 7.80 (dd, J=2.0, 0.9 Hz, 1H), 7.34 (s, 1H), 7.02 (dd, J=7.2, 2.0 Hz, 1H), 6.87 (s, 1H), 4.69-4.58 (m, 1H), 4.06 (s, 1H), 2.45 (s, 3H), 1.96-1.90 (m, 1H), 1.88-1.76 (m, 2H), 1.65-1.56 (m, 2H), 1.42-1.22 (m, 4H), 0.92 (s, 3H), 0.88-0.76 (m, 4H).

### Example 152: N-[5-[5-[(1R,3S)-3-aminocyclopentoxy]-2-methyl-4-pyridyl]pyrazolo[1,5-a]pyridin-2-yl]cyclopropanecarboxamide

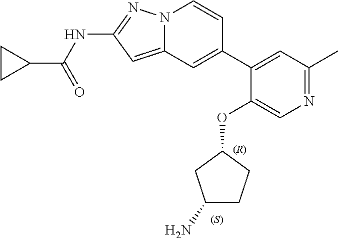

The title compound (66 mg, 52%) was prepared as described in Example 136 where (1R,3S)-3-aminocyclopentan-1-ol was used instead of endo-7-hydroxy-3-oxa-9-azabicyclo[3.3.1]nonane hydrochloride. MS (ESI): mass calcd. for C₂₂H₂₅N₅O₂, 391.2. m/z found, 392.3 [M+H]⁺. ¹H NMR (400 MHz, DMSO-d₆) δ 11.07 (s, 1H), 8.55 (dt, J=7.3, 1.0 Hz, 1H), 8.26 (s, 1H), 7.85 (dd, J=2.0, 0.9 Hz, 1H), 7.34 (s, 1H), 7.09 (dd, J=7.2, 2.0 Hz, 1H), 6.88 (s, 1H), 4.87 (dd, J=6.5, 3.1 Hz, 1H), 3.18 (dd, J=12.4, 5.9 Hz, 1H), 2.45 (s, 3H), 2.30-2.21 (m, 1H), 1.99-1.69 (m, 5H), 1.49-1.33 (m, 2H), 0.82 (ddt, J=8.2, 4.9, 2.9 Hz, 4H).

### Example 153: N-[5-[5-[(1R,3R)-3-aminocyclopentoxy]-2-methyl-4-pyridyl]pyrazolo[1,5-a]pyridin-2-yl]cyclopropanecarboxamide

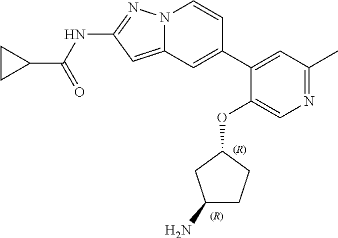

The title compound (40 mg, 32%) was prepared as described in Example 136 where (1R,3R)-3-aminocyclopentan-1-ol was used instead of endo-7-hydroxy-3-oxa-9-azabicyclo[3.3.1]nonane hydrochloride·MS (ESI): mass calcd. for C₂₂H₂₅N₅O₂, 391.2. m/z found, 392.2 [M+H]⁺. ¹H NMR (400 MHz, CDCl₃) δ 8.41 (s, 1H), 8.28 (dt, J=7.1, 0.9 Hz, 1H), 8.24 (s, 1H), 7.65 (dd, J=2.0, 0.9 Hz, 1H), 7.18 (s, 1H), 7.02 (s, 1H), 6.95 (dd, J=7.2, 1.9 Hz, 1H), 4.94 (tt, J=6.0, 2.7 Hz, 1H), 3.58 (p, J=6.7 Hz, 1H), 2.56 (s, 3H), 2.32-2.19 (m, 1H), 2.19-2.11 (m, 1H), 2.06-1.99 (m, 1H), 1.89-1.79 (m, 1H), 1.72 (dt, J=13.7, 6.7 Hz, 1H), 1.61 (s, 1H), 1.40 (ddt, J=12.7, 8.9, 6.3 Hz, 1H), 1.21-1.12 (m, 2H), 0.99-0.88 (m, 2H).

### Example 154: N-[5-[5-[(1-aminocyclobutyl)methoxy]-2-methyl-4-pyridyl]pyrazolo[1,5-a]pyridin-2-yl]cyclopropanecarboxamide

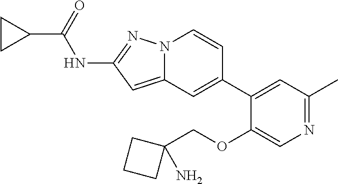 [SMILES: Cc1cc(-c2ccn3nc(NC(=O)C4CC4)cc3c2)c(OCC2(N)CCC2)cn1]

The title compound (201 mg, 52%) was prepared as described in Example 136 where (1-aminocyclobutyl)methanol was used instead of endo-7-hydroxy-3-oxa-9-azabicyclo[3.3.1]nonane hydrochloride. MS (ESI): mass calcd. for C₂₂H₂₅N₅O₂, 391.2. m/z found, 392.3 [M+H]⁺. ¹H NMR (400 MHz, DMSO-d₆) δ 11.10 (s, 1H), 8.57 (d, J=7.2 Hz, 1H), 8.36 (s, 1H), 7.88 (d, J=0.8 Hz, 1H), 7.36 (s, 1H), 7.08 (dd, J=2.0, 7.2 Hz, 1H), 6.87 (s, 1H), 3.99 (s, 2H), 2.47 (s, 3H), 2.08-1.98 (m, 2H), 1.96-1.88 (m, 1H), 1.84-1.65 (m, 5H), 1.63-1.51 (m, 1H), 0.86-0.79 (m, 4H).

### Example 155: N-[5-[5-[(1-aminocyclopentyl)methoxy]-2-methyl-4-pyridyl]pyrazolo[1,5-a]pyridin-2-yl]cyclopropanecarboxamide

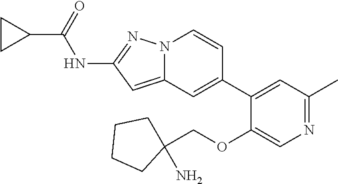 [SMILES: Cc1cc(-c2ccn3nc(NC(=O)C4CC4)cc3c2)c(OCC2(N)CCCC2)cn1]

The title compound (104 mg, 26%) was prepared as described in Example 136 where (1-aminocyclopentyl)methanol was used instead of endo-7-hydroxy-3-oxa-9-azabicyclo[3.3.1]nonane hydrochloride. MS (ESI): mass calcd. for C₂₃H₂₇N₅O₂, 405.2. m/z found, 406.3 [M+H]⁺. ¹H NMR (400 MHz, DMSO-d₆) δ 11.10 (s, 1H), 8.58 (d, J=6.8 Hz, 1H), 8.32 (s, 1H), 7.86 (d, J=1.2 Hz, 1H), 7.36 (s, 1H), 7.07 (dd, J=2.0, 7.2 Hz, 1H), 6.88 (s, 1H), 3.93 (s, 2H), 2.46 (s, 3H), 1.98-1.89 (m, 1H), 1.76-1.65 (m, 2H), 1.64-1.54 (m, 2H), 1.54-1.42 (m, 4H), 1.40-1.32 (m, 2H), 0.86-0.79 (m, 4H).

### Example 156: N-[5-[2-methyl-5-[[1-(methylamino)cyclobutyl]methoxy]-4-pyridyl]pyrazolo[1,5-a]pyridin-2-yl]cyclopropanecarboxamide

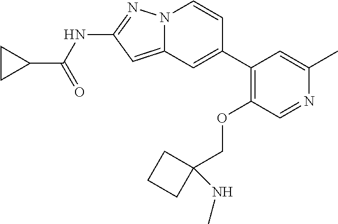 [SMILES: CNC1(COc2cnc(C)cc2-c2ccn3nc(NC(=O)C4CC4)cc3c2)CCC1]

The title compound (57 mg, 22%) was prepared as described in Example 136 where (1-(methylamino)cyclobutyl)methanol was used instead of endo-7-hydroxy-3-oxa-9-azabicyclo[3.3.1]nonane hydrochloride. MS (ESI): mass calcd. for C₂₃H₂₇N₅O₂, 405.2. m/z found, 406.3 [M+H]⁺. ¹H NMR (400 MHz, DMSO-d₆) δ 11.09 (s, 1H), 8.56 (d, J=7.2 Hz, 1H), 8.39 (s, 1H), 7.91 (s, 1H), 7.36 (s, 1H), 7.08 (dd, J=1.6, 7.2 Hz, 1H), 6.86 (s, 1H), 4.08 (s, 2H), 2.47 (s, 3H), 2.14 (s, 3H), 1.95-1.82 (m, 6H), 1.77-1.61 (m, 2H), 0.90-0.76 (m, 4H).

### Example 157: N-[5-[5-[[1-(cyclopropylamino)cyclobutyl]methoxy]-2-methyl-4-pyridyl]pyrazolo[1,5-a]pyridin-2-yl]cyclopropanecarboxamide

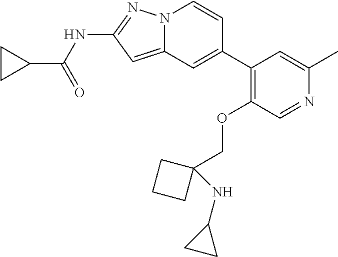 [SMILES: Cc1cc(-c2ccn3nc(NC(=O)C4CC4)cc3c2)c(OCC2(NC3CC3)CCC2)cn1]

The title compound (18 mg, 6.4%) was prepared as described in Example 136 where (1-(cyclopropylamino)cyclobutyl)methanol was used instead of endo-7-hydroxy-3-oxa-9-azabicyclo[3.3.1]nonane hydrochloride. MS (ESI): mass calcd. for C₂₅H₂₉N₅O₂, 431.2. m/z found, 432.3 [M+H]⁺. ¹H NMR (400 MHz, DMSO-d₆) δ 11.09 (s, 1H), 8.54 (d, J=7.2 Hz, 1H), 8.38 (s, 1H), 7.96 (d, J=0.8 Hz, 1H), 7.38 (s, 1H), 7.11 (dd, J=2.0, 7.2 Hz, 1H), 6.86 (s, 1H), 4.17 (s, 2H), 2.47 (s, 3H), 2.42 (br s, 1H), 2.06-1.88 (m, 6H), 1.74-1.64 (m, 2H), 0.87-0.79 (m, 4H), 0.33-0.27 (m, 2H), 0.15-0.09 (m, 2H).

### Example 158: trans-N-[5-[5-(4-hydroxycyclohexoxy)-2-methyl-4-pyridyl]pyrazolo[1,5-a]pyridin-2-yl]cyclopropanecarboxamide

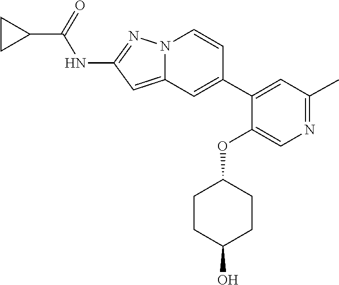 [SMILES: Cc1cc(-c2ccn3nc(NC(=O)C4CC4)cc3c2)c(O[C@H]2CC[C@H](O)CC2)cn1]

The title compound (28 mg, 11%) was prepared as described in Example 136 where (trans)-cyclohexane-1,4-diol was used instead of endo-7-hydroxy-3-oxa-9-azabicyclo[3.3.1]nonane hydrochloride. MS (ESI): mass calcd. for C₂₃H₂₆N₄O₃, 406.2. m/z found, 407.2 [M+H]⁺. ¹H NMR (400 MHz, DMSO-d₆) δ 11.09 (s, 1H), 8.56 (d, J=7.2 Hz, 1H), 8.35 (s, 1H), 7.80 (d, J=0.8 Hz, 1H), 7.34 (s, 1H), 7.03 (dd, J=1.6, 7.2 Hz, 1H), 6.88 (s, 1H), 4.54 (d, J=4.0 Hz, 1H), 4.46-4.37 (m, 1H), 3.56-3.44 (m, 1H), 2.45 (s, 3H), 2.02-1.88 (m, 3H), 1.78-1.67 (m, 2H), 1.47-1.34 (m, 2H), 1.33-1.20 (m, 2H), 0.88-0.76 (m, 4H).

### Example 159: (*R)—N-[5-[5-(2-amino-3,3,3-trifluoro-2-methyl-propoxy)-2-methyl-4-pyridyl]pyrazolo[1,5-a]pyridin-2-yl]cyclopropanecarboxamide

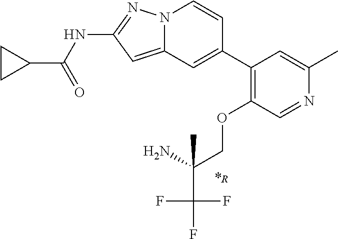

The racemic (rac-)-N-[5-[5-(2-amino-3,3,3-trifluoro-2-methyl-propoxy)-2-methyl-4-pyridyl]pyrazolo[1,5-a]pyridin-2-yl]cyclopropanecarboxamide (130 mg, 46%) was prepared as described in Example 136 where (rac-)-2-amino-3,3,3-trifluoro-2-methylpropan-1-ol was used instead of endo-7-hydroxy-3-oxa-9-azabicyclo[3.3.1]nonane hydrochloride. The enantiomers of (rac-)-N-[5-[5-(2-amino-3,3,3-trifluoro-2-methyl-propoxy)-2-methyl-4-pyridyl]pyrazolo[1,5-a]pyridin-2-yl]cyclopropanecarboxamide were separated by SFC over DAICEL CHIRALPAK AD-H 250 mm×30 mm, 5 μm (eluent: 45% to 45% (v/v) supercritical CO₂ in EtOH and H₂O with 0.1% NH₃) to afford (*R)—N-[5-[5-(2-amino-3,3,3-trifluoro-2-methyl-propoxy)-2-methyl-4-pyridyl]pyrazolo[1,5-a]pyridin-2-yl]cyclopropanecarboxamide (title compound, 44.10 mg) and (*S)—N-[5-[5-(2-amino-3,3,3-trifluoro-2-methyl-propoxy)-2-methyl-4-pyridyl]pyrazolo[1,5-a]pyridin-2-yl]cyclopropanecarboxamide (Example 160, 32.10 mg) as white solids. MS (ESI): mass calcd. for C₂₁H₂₂F₃N₅O₂, 433.2. m/z found, 434.2 [M+H]⁺. ¹H NMR (400 MHz, DMSO-d₆) δ 11.12 (s, 1H), 8.57 (d, J=7.2 Hz, 1H), 8.36 (s, 1H), 7.84 (d, J=0.8 Hz, 1H), 7.38 (s, 1H), 7.04 (dd, J=2.0, 7.2 Hz, 1H), 6.88 (s, 1H), 4.13 (d, J=10.0 Hz, 1H), 4.10 (d, J=9.6 Hz, 1H), 2.47 (s, 3H), 2.13 (s, 2H), 1.99-1.90 (m, 1H), 1.22 (s, 3H), 0.88-0.79 (m, 4H). ¹⁹F NMR (376 MHz, DMSO-d₆) δ −78.2 (s, 3F).

### Example 160: (*S)—N-[5-[5-(2-amino-3,3,3-trifluoro-2-methyl-propoxy)-2-methyl-4-pyridyl]pyrazolo[1,5-a]pyridin-2-yl]cyclopropanecarboxamide

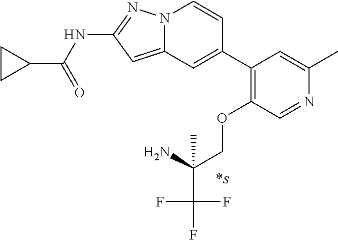

The title compound was isolated from the separation of the racemate in Example 159. MS (ESI): mass calcd. for C₂₁H₂₂F₃N₅O₂, 433.2. m/z found, 434.2 [M+H]⁺. ¹H NMR (400 MHz, DMSO-d₆) δ 11.11 (s, 1H), 8.58 (d, J=7.2 Hz, 1H), 8.36 (s, 1H), 7.86-7.81 (m, 1H), 7.38 (s, 1H), 7.04 (dd, J=2.0, 7.2 Hz, 1H), 6.88 (s, 1H), 4.13 (d, J=10.0 Hz, 1H), 4.09 (d, J=10.0 Hz, 1H), 2.47 (s, 3H), 2.13 (s, 2H), 1.98-1.88 (m, 1H), 1.21 (s, 3H), 0.86-0.78 (m, 4H). ¹⁹F NMR (376 MHz, DMSO-d₆) δ −78.2 (s, 3F).

### Example 161: 7-endo-N-(5-(5-(((1R,5S,7s)-9-ethyl-3-oxa-9-azabicyclo[3.3.1]nonan-7-yl)oxy)-2-methylpyridin-4-yl)pyrazolo[1,5-a]pyridin-2-yl)cyclopropanecarboxamide

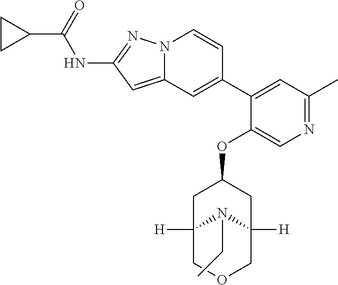 [SMILES: CCN1[C@@H]2COC[C@H]1C[C@@H](Oc1cnc(C)cc1-c1ccn3nc(NC(=O)C4CC4)cc3c1)C2]

A solution of iodoethane (0.195 mL, 2.4 mmol) in 20 mL of acetonitrile was prepared in advance; added 2 mL of this solution (with −0.24 mmol iodoethane) to a vial containing endo-N-(5-(5-(((1R,5S,7s)-3-oxa-9-azabicyclo[3.3.1]nonan-7-yl)oxy)-2-methylpyridin-4-yl)pyrazolo[1,5-a]pyridin-2-yl)cyclopropanecarboxamide (Example 136, 104 mg, 0.24 mmol). This was followed by the addition of DIPEA (0.207 mL, 1.2 mmol) and heating at 50° C. for 16 hours. The reaction was worked up with ethyl acetate, water, and brine. The organic extracts were dried with sodium sulfate and purified over FCC (0-20% 2M NH₃ in MeOH/DCM) to provide the title compound (21 mg, 19%) as a white solid. MS (ESI): mass calcd. for C₂₆H₃₁N₅O₃, 461.2. m/z found, 462.3 [M+H]⁺. ¹H NMR (500 MHz, DMSO-d₆) δ 11.06 (s, 1H), 8.53-8.47 (m, 1H), 8.30-8.23 (m, 1H), 8.17 (s, 1H), 7.39 (s, 1H), 7.26 (dd, J=7.3, 2.1 Hz, 1H), 6.82 (s, 1H), 4.77-4.65 (m, 1H), 3.81-3.68 (m, 2H), 3.56 (d, J=10.6 Hz, 2H), 2.81-2.70 (m, 2H), 2.61 (q, J=7.1 Hz, 2H), 2.45 (s, 3H), 2.38-2.29 (m, 2H), 1.98-1.87 (m, 1H), 1.62 (d, J=15.2 Hz, 2H), 0.98 (t, J=7.1 Hz, 3H), 0.87-0.78 (m, 4H).

### Example 162: 5-[5-[(1-amino-3,3-difluoro-cyclobutyl)methoxy]-2-methyl-4-pyridyl]-N-(1-methylpyrazol-3-yl)pyrazolo[1,5-a]pyridin-2-amine

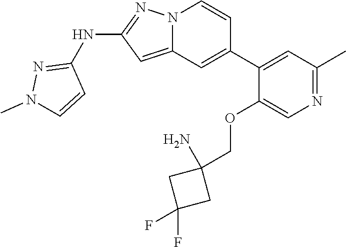

Step A: 5-(5-((1-amino-3,3-difluorocyclobutyl)methoxy)-2-methylpyridin-4-yl)pyrazolo[1,5-a]pyridin-2-amine. The title compound was prepared in a manner similar to that in Example 136 using 1-amino-3,3-difluoro-cyclobutanemethanol hydrochloride (611 mg, 3.52 mmol) instead of endo-7-hydroxy-3-oxa-9-azabicyclo[3.3.1]nonane hydrochloride and Intermediate 8 (569 mg, 2.35 mmol) instead of Intermediate 6 to provide the product (157 mg, 19%) as a tan foam. MS (ESI): mass calcd. for C₈H₁₉F₂N₅O, 359.2. m/z found, 360.1 [M+H]⁺. ¹H NMR (500 MHz, DMSO-d₆) δ 8.32 (s, 1H), 8.28 (d, J=7.1 Hz, 1H), 7.56-7.52 (m, 1H), 7.33 (s, 1H), 6.77 (dd, J=7.2, 2.0 Hz, 1H), 5.69 (s, 1H), 5.33 (s, 2H), 4.00 (s, 2H), 2.75-2.64 (m, 2H), 2.46 (s, 3H), 2.42-2.31 (m, 2H), 2.04 (br s, 2H).

Step B: 5-[5-[(1-amino-3,3-difluoro-cyclobutyl)methoxy]-2-methyl-4-pyridyl]-N-(1-methylpyrazol-3-yl)pyrazolo[1,5-a]pyridin-2-amine. The title compound was prepared in a manner similar to that in Example 116, Step A, using 5-(5-((1-amino-3,3-difluorocyclobutyl)methoxy)-2-methylpyridin-4-yl)pyrazolo[1,5-a]pyridin-2-amine (35 mg, 0.1 mmol) instead of tert-butyl (1R,5S,7s)-7-((4-(2-aminopyrazolo[1,5-a]pyridin-5-yl)-6-methylpyridin-3-yl)oxy)-3-oxa-9-azabicyclo[3.3.1]nonane-9-carboxylate and 3-bromo-1-methyl-1H-pyrazole (45 mg, 0.28 mmol) instead of 4-bromo-1-(difluoromethyl)-1H-pyrazole and Brettphos-Pd-G2 (16 mg, 0.02 mmol) instead of BuBrettPhos-Pd-G3 and the addition of Brettphos (10 mg, 0.02 mmol) to give the product as a white solid (17 mg, 41%). MS (ESI): mass calcd. for C₂₂H₂₃F₂N₇O, 439.2. m/z found, 440.3 [M+H]⁺. ¹H NMR (400 MHz, DMSO-d₆) δ 9.05 (s, 1H), 8.42 (d, J=7.1 Hz, 1H), 8.33 (s, 1H), 7.68 (d, J=2.0 Hz, 1H), 7.49 (d, J=2.2 Hz, 1H), 7.34 (s, 1H), 6.88 (dd, J=7.2, 2.0 Hz, 1H), 6.39 (s, 1H), 6.07 (d, J=2.2 Hz, 1H), 4.01 (s, 2H), 3.73 (s, 3H), 2.76-2.63 (m, 2H), 2.47 (s, 3H), 2.43-2.30 (m, 2H), 2.04 (s, 2H).

### Example 163: (1r,4r)-1-methyl-4-((6-methyl-4-(2-((1-methyl-1H-pyrazol-4-yl)amino)pyrazolo[1,5-a]pyridin-5-yl)pyridin-3-yl)oxy)cyclohexan-1-ol

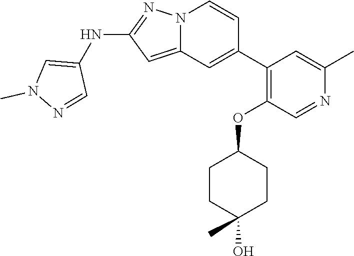

Step A: (1r,4r)-4-((4-(2-aminopyrazolo[1,5-a]pyridin-5-yl)-6-methylpyridin-3-yl)oxy)-1-methylcyclohexan-1-ol. The title compound was prepared in a manner similar to that in Example 136 using (1r,4r)-1-methylcyclohexane-1,4-diol (960 mg, 7.37 mmol) instead of endo-7-hydroxy-3-oxa-9-azabicyclo[3.3.1]nonane hydrochloride and Intermediate 8 (1.06 g, 4.38 mmol) instead of Intermediate 6 and NMP (20 mL) instead of DMA to provide the product (857 mg, 56%).

Step B: (1r,4r)-1-methyl-4-((6-methyl-4-(2-((1-methyl-1H-pyrazol-4-yl)amino)pyrazolo[1,5-a]pyridin-5-yl)pyridin-3-yl)oxy)cyclohexan-1-ol. The title compound was prepared in a manner similar to that in Example 116, Step A, using (1r,4r)-4-((4-(2-aminopyrazolo[1,5-a]pyridin-5-yl)-6-methylpyridin-3-yl)oxy)-1-methylcyclohexan-1-ol (107 mg, 0.30 mmol)) instead of tert-butyl (1R,5S,7s)-7-((4-(2-aminopyrazolo[1,5-a]pyridin-5-yl)-6-methylpyridin-3-yl)oxy)-3-oxa-9-azabicyclo[3.3.1]nonane-9-carboxylate and 4-bromo-1-methyl-1H-pyrazole (97 mg, 0.60 mmol) instead of 4-bromo-1-(difluoromethyl)-1H-pyrazole to give the product (50 mg, 38%). MS (ESI): mass calcd. for C₂₄H₂₈N₆O₂, 432.2. m/z found, 433.2 [M+H]⁺. ¹H NMR (400 MHz, DMSO-d₆) δ 8.47 (d, J=7.1 Hz, 2H), 8.44 (s, 1H), 8.34 (s, 1H), 7.77 (d, J=0.8 Hz, 1H), 7.60-7.55 (m, 1H), 7.37 (d, J=0.8 Hz, 1H), 7.32 (s, 1H), 6.81 (dd, J=7.2, 2.0 Hz, 1H), 5.91 (d, J=0.7 Hz, 1H), 4.67-4.58 (m, 1H), 4.06 (br s, 1H), 3.81 (s, 3H), 2.45 (s, 3H), 1.88-1.75 (m, 2H), 1.67-1.55 (m, 2H), 1.46-1.34 (m, 2H), 1.32-1.22 (m, 2H), 0.94 (s, 3H).

### Example 164: N-(5-(2-methyl-5-(((1S,4S,7R)-5-methyl-2-oxa-5-azabicyclo[2.2.1]heptan-7-yl)oxy)pyridin-4-yl)pyrazolo[1,5-a]pyridin-2-yl)cyclopropanecarboxamide

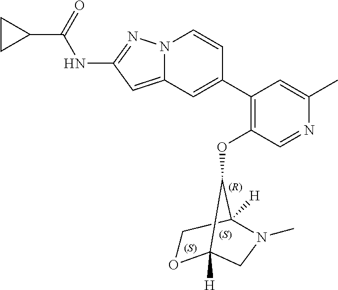

To a 20 mL vial was charged N-(5-(5-(((1S,4S,7R)-2-oxa-5-azabicyclo[2.2.1]heptan-7-yl)oxy)-2-methylpyridin-4-yl)pyrazolo[1,5-a]pyridin-2-yl)cyclopropanecarboxamide (Example 165, 45 mg, 0.11 mmol), methanol (2.2 mL), paraformaldehyde (15 mg, 0.17 mmol), and sodium cyanoborohydride (21 mg, 0.33 mmol). The resulting mixture was stirred for 20 min then portioned between ethyl acetate and water. The layers were separated and the organics dried with MgSO₄, filtered, then concentrated to dryness. The residue was purified by flash column chromatography (0-25% 2 N NH₃/methanol in dichloromethane) and the fractions containing product were combined and concentrated to afford the title compound (37 mg, 80%) as a yellow solid. MS (ESI): mass calcd. for C₂₃H₂₅N₅O₃, 419.2. m/z found, 420.2 [M+H]⁺. ¹H NMR (500 MHz, CD₃OD) δ 8.44-8.39 (m, 2H), 7.78-7.74 (m, 1H), 7.41-7.39 (m, 1H), 7.04 (dd, J=7.2, 2.0 Hz, 1H), 6.90 (s, 1H), 4.94 (d, J=2.3 Hz, 1H), 4.33-4.29 (m, 1H), 4.15 (d, J=8.4 Hz, 1H), 3.85 (dd, J=8.3, 2.0 Hz, 1H), 3.47 (t, J=2.1 Hz, 1H), 3.12 (dd, J=10.7, 1.8 Hz, 1H), 2.65 (dd, J=10.8, 0.8 Hz, 1H), 2.54 (s, 3H), 2.44 (s, 3H), 1.91-1.83 (m, 1H), 1.03-0.97 (m, 2H), 0.93-0.87 (m, 2H).

### Example 165: N-(5-(5-(((1S,4S,7R)-2-oxa-5-azabicyclo[2.2.1]heptan-7-yl)oxy)-2-methylpyridin-4-yl)pyrazolo[1,5-a]pyridin-2-yl)cyclopropanecarboxamide

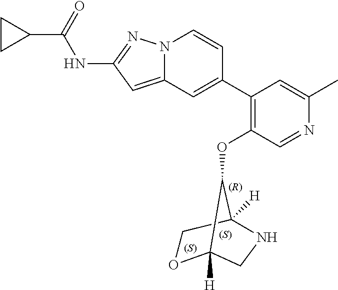

Step A: tert-butyl (1S,4S,7R)-7-((4-(2-(cyclopropanecarboxamido)pyrazolo[1,5-a]pyridin-5-yl)-6-methylpyridin-3-yl)oxy)-2-oxa-5-azabicyclo[2.2.1]heptane-5-carboxylate. To a 40 mL vial, was charged NaH (60% dispersion in mineral oil) (96.6 mg, 2.42 mmol) and DMA (1.9 mL) followed by tert-butyl (1S,4S,7R)-7-hydroxy-2-oxa-5-azabicyclo[2.2.1]heptane-5-carboxylate (260 mg, 1.21 mmol). The resulting suspension was heated to 50° C. for 5 min to promote full deprotonation. Intermediate 6 (150 mg, 0.483 mmol) was added in a single portion. The reaction was stirred 6 hours at 50-90° C. until >90% conversion was achieved. The reaction was cooled to room temperature at which point the reaction mixture was quenched into stirring water (20 mL). The organics were extracted into ethyl acetate, washed with saturated sodium chloride, dried (Na₂SO₄) then concentrated to dryness. The residue was purified by flash column chromatography (25-100% EtOAc in hexanes) to afford the title compound.

Step B: N-(5-(5-(((1S,4S,7R)-2-oxa-5-azabicyclo[2.2.1]heptan-7-yl)oxy)-2-methylpyridin-4-yl)pyrazolo[1,5-a]pyridin-2-yl)cyclopropanecarboxamide. To a vial containing a solution of tert-butyl (1S,4S,7R)-7-((4-(2-(cyclopropanecarboxamido)pyrazolo[1,5-a]pyridin-5-yl)-6-methylpyridin-3-yl)oxy)-2-oxa-5-azabicyclo[2.2.1]heptane-5-carboxylate (120 mg, 0.24 mmol) in DCE (1.9 mL) was added trifluoracetic acid (0.37 mL, 4.8 mmol) at 23° C. and after 20 minutes the solvent was removed in vacuo. The residue was diluted with dichloromethane (6 mL) followed by saturated aq. K₂CO₃ (3 mL). The layers were separated and the aqueous was extracted with 20% methanol/dichloromethane (3×10 mL). The combined organics were dried with Na₂SO₄, filtered and concentrated in vacuo. The residue was purified by flash column chromatography (0-25% 2 N NH₃/methanol in dichloromethane) and the fractions containing product were combined and concentrated to afford the title compound (120 mg, 49%). MS (ESI): mass calcd. for C₂₂H₂₃N₅O₃, 405.2. m/z found, 406.2 [M+H]⁺. ¹H NMR (500 MHz, CD₃OD) δ 8.42-8.38 (m, 2H), 7.77-7.74 (m, 1H), 7.39 (s, 1H), 7.04 (dd, J=7.2, 2.0 Hz, 1H), 6.90 (s, 1H), 4.87 (d, J=2.4 Hz, 1H), 4.35-4.32 (m, 1H), 4.02 (dd, J=7.8, 2.0 Hz, 1H), 3.87 (d, J=7.8 Hz, 1H), 3.54 (t, J=2.2 Hz, 1H), 3.13 (dd, J=11.1, 1.7 Hz, 1H), 3.01-2.94 (m, 1H), 2.54 (s, 3H), 1.90-1.82 (m, 1H), 1.02-0.96 (m, 2H), 0.93-0.87 (m, 2H).

### Example 166: N-[5-[2-methyl-5-[[(2R)-morpholin-2-yl]methoxy]-4-pyridyl]pyrazolo[1,5-a]pyridin-2-yl]cyclopropanecarboxamide

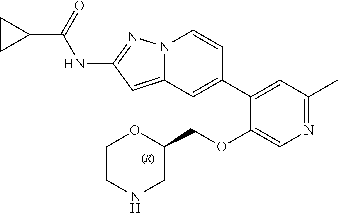

To a 50 mL flask containing tert-butyl (R)-2-(hydroxymethyl)morpholine-4-carboxylate (210 mg, 0.967 mmol) was added HCl (4M in dioxane) (0.806 mL, 4 M, 3.222 mmol). The reaction was stirred for 2 h. The reaction mixture was concentrated to dryness. The solid was pumped under vacuo for 1 h. Then, the solid was dissolved with DMA (1.289 mL, 0.25 M, 0.322 mmol). To the solution was added NaH (60% dispersion in mineral oil) (129 mg, 3.22 mmol) and stirred for 5 min. To this mixture was then added N-(5-(5-fluoro-2-methylpyridin-4-yl)pyrazolo[1,5-a]pyridin-2-yl)cyclopropanecarboxamide (Intermediate 6, 100 mg, 0.322 mmol). The reaction was stirred at 90° C. overnight. The crude reaction mixture was diluted with EtOAc and washed with brine 3×. The organics were collected, dried over MgSO₄, filtered, and concentrated under reduced pressure. The product was absorbed onto silica gel and purified by FCC with 3-10% of 2M NH₃ in MeOH-DCM to afford the title compound (50 mg, 38%). MS (ESI): mass calcd. for C₂₂H₂₅N₅O₃, 407.2. m/z found, 408.2 [M+H]⁺. ¹H NMR (400 MHz, CDCl₃) δ 8.29 (d, J=12.1 Hz, 3H), 7.72 (dd, J=2.0, 0.9 Hz, 1H), 7.19 (s, 1H), 6.99 (dd, J=7.2, 2.0 Hz, 2H), 4.12 (t, J=4.9 Hz, 1H), 4.05 (dd, J=9.9, 4.8 Hz, 1H), 3.94 (d, J=11.4 Hz, 1H), 3.91-3.84 (m, 1H), 3.74-3.66 (m, 1H), 2.98 (dd, J=12.1, 2.4 Hz, 1H), 2.92-2.85 (m, 2H), 2.76 (dd, J=12.1, 10.4 Hz, 1H), 2.58 (s, 3H), 1.20-1.12 (m, 2H), 0.98-0.90 (m, 2H).

### Example 167: N-[5-[5-[[(2S)-5,5-dimethylmorpholin-2-yl]methoxy]-2-methyl-4-pyridyl]pyrazolo[1,5-a]pyridin-2-yl]cyclopropanecarboxamide

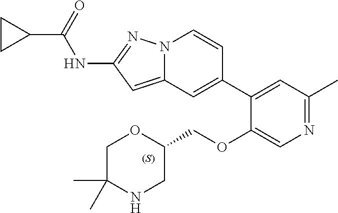

The title compound (76 mg, 54%) was prepared as described in Example 166 where tert-butyl (S)-2-(hydroxymethyl)-5,5-dimethylmorpholine-4-carboxylate was used instead of tert-butyl (R)-2-(hydroxymethyl)morpholine-4-carboxylate. MS (ESI): mass calcd. for C₂₄H₂₉N₅O₃, 435.2. m/z found, 436.3 [M+H]⁺. ¹H NMR (400 MHz, CDCl₃) δ 8.25 (d, J=6.7 Hz, 3H), 7.68 (dd, J=2.0, 0.9 Hz, 1H), 7.17 (s, 1H), 7.01-6.91 (m, 2H), 4.17-4.04 (m, 2H), 3.69 (ddd, J=10.3, 7.8, 4.7 Hz, 1H), 3.59 (d, J=11.1 Hz, 1H), 3.30 (d, J=11.1 Hz, 1H), 3.00 (dd, J=12.8, 10.8 Hz, 1H), 2.76 (dd, J=12.8, 2.9 Hz, 1H), 2.55 (s, 3H), 1.57 (s, 1H), 1.21 (s, 3H), 1.18-1.11 (m, 2H), 1.01 (s, 3H), 0.92 (dt, J=7.8, 3.5 Hz, 2H).

### Example 168: N-[5-[2-methyl-5-[[(2S,5R)-5-methylmorpholin-2-yl]methoxy]-4-pyridyl]pyrazolo[1,5-a]pyridin-2-yl]cyclopropanecarboxamide

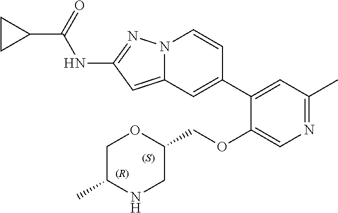

The title compound (76 mg, 37%) was prepared as described in Example 165 where tert-butyl (2S,5R)-2-(hydroxymethyl)-5-methylmorpholine-4-carboxylate was used instead of used instead of tert-butyl (1S,4S,7R)-7-hydroxy-2-oxa-5-azabicyclo[2.2.1]heptane-5-carboxylate. MS (ESI): mass calcd. for C₂₃H₂₇N₅O₃, 421.2. m/z found, 422.3 [M+H]⁺. ¹H NMR (400 MHz, DMSO-d₆) δ 11.08 (s, 1H), 8.55 (d, J=7.2 Hz, 1H), 8.37 (s, 1H), 7.87 (dd, J=2.0, 0.9 Hz, 1H), 7.36 (s, 1H), 7.08 (dd, J=7.2, 2.0 Hz, 1H), 6.88 (s, 1H), 4.36-4.21 (m, 2H), 3.81-3.71 (m, 1H), 3.51 (dd, J=11.0, 3.2 Hz, 1H), 3.38 (dd, J=11.0, 5.6 Hz, 1H), 2.89-2.66 (br s, 3H), 2.47 (s, 3H), 2.36-2.14 (m, 1H), 1.99-1.88 (m, 1H), 0.98 (d, J=6.6 Hz, 3H), 0.88-0.77 (m, 4H).

### Example 169: N-[5-[5-[(3-fluoro-1-methyl-azetidin-3-yl)methoxy]-2-methyl-4-pyridyl]pyrazolo[1,5-a]pyridin-2-yl]cyclopropanecarboxamide

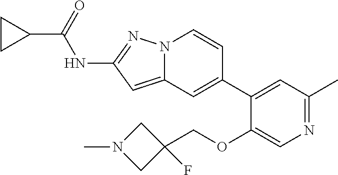 [SMILES: Cc1cc(-c2ccn3nc(NC(=O)C4CC4)cc3c2)c(OCC2(F)CN(C)C2)cn1]

The title compound (83 mg, 25%) was prepared as described in Example 164 where (3-fluoroazetidin-3-yl)methanol was used instead of tert-butyl (1S,4S,7R)-7-hydroxy-2-oxa-5-azabicyclo[2.2.1]heptane-5-carboxylate. MS (ESI): mass calcd. for C₂₂H₂₄FN₅O₂, 409.2. m/z found, 410.3 [M+H]⁺. ¹H NMR (600 MHz, DMSO-d₆) δ 11.07 (s, 1H), 8.56 (d, J=7.2 Hz, 1H), 8.38 (s, 1H), 7.82 (d, J=1.9 Hz, 1H), 7.38 (s, 1H), 7.03 (dd, J=7.2, 1.9 Hz, 1H), 6.87 (s, 1H), 4.43 (d, J=23.9 Hz, 2H), 3.52-3.43 (m, 2H), 3.13-3.04 (m, 2H), 2.47 (s, 3H), 2.28 (s, 3H), 1.98-1.90 (m, 1H), 0.86-0.79 (m, 4H).

### Example 170: trans-N-[5-[5-(3-aminocyclobutoxy)-2-methyl-4-pyridyl]pyrazolo[1,5-a]pyridin-2-yl]cyclopropanecarboxamide

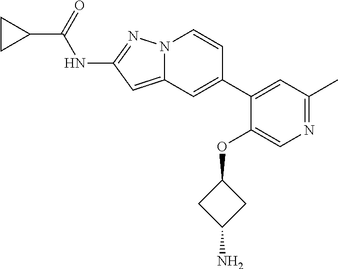 [SMILES: Cc1cc(-c2ccn3nc(NC(=O)C4CC4)cc3c2)c(O[C@H]2C[C@H](N)C2)cn1]

The title compound (37 mg, 30%) was prepared as described in Example 166 where tert-butyl ((1r,3r)-3-hydroxycyclobutyl)carbamate was used instead of tert-butyl (R)-2-(hydroxymethyl)morpholine-4-carboxylate. MS (ESI): mass calcd. for C₂₁H₂₅N₅O₂, 377.2. m/z found, 378.1 [M+H]⁺. ¹H NMR (400 MHz, DMSO-d₆) δ 11.07 (s, 1H), 8.56 (dt, J=7.3, 1.0 Hz, 1H), 8.06 (s, 1H), 7.81 (dd, J=2.0, 0.9 Hz, 1H), 7.35 (s, 1H), 7.05 (dd, J=7.3, 2.0 Hz, 1H), 6.89 (s, 1H), 4.97 (ddd, J=10.5, 6.7, 4.1 Hz, 1H), 3.58-3.49 (m, 1H), 2.25 (ddd, J=11.9, 7.7, 4.1 Hz, 2H), 2.18-2.04 (m, 2H), 1.96-1.81 (m, 2H), 0.89-0.70 (m, 4H).

### Example 171: cis-N-[5-[5-[(3-aminocyclobutyl)methoxy]-2-methyl-4-pyridyl]pyrazolo[1,5-a]pyridin-2-yl]cyclopropanecarboxamide

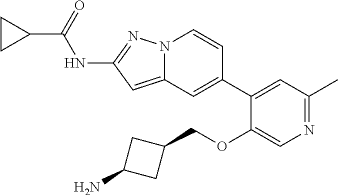 [SMILES: Cc1cc(-c2ccn3nc(NC(=O)C4CC4)cc3c2)c(OC[C@H]2C[C@@H](N)C2)cn1]

The title compound (28 mg, 20%) was prepared as described in Example 166 where tert-butyl ((1s,3s)-3-hydroxycyclobutyl)carbamate was used instead of tert-butyl (R)-2-(hydroxymethyl)morpholine-4-carboxylate. MS (ESI): mass calcd. for C₂₂H₂₅N₅O₂, 391.2. m/z found, 392.2 [M+H]⁺. ¹H NMR (400 MHz, DMSO-d₆) δ 11.08 (s, 1H), 8.58 (dd, J=7.2, 1.0 Hz, 1H), 8.30 (s, 1H), 7.87-7.79 (m, 1H), 7.35 (s, 1H), 7.04 (dd, J=7.2, 2.0 Hz, 1H), 6.88 (s, 1H), 4.03 (d, J=5.9 Hz, 2H), 3.15 (s, 1H), 2.46 (s, 3H), 2.20 (tt, J=14.9, 8.0 Hz, 4H), 1.92 (td, J=7.9, 7.4, 4.0 Hz, 1H), 1.73 (d, J=10.0 Hz, 1H), 1.50-1.39 (m, 2H), 0.87-0.78 (m, 4H).

### Example 172: 2-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-N-methylisonicotinamide

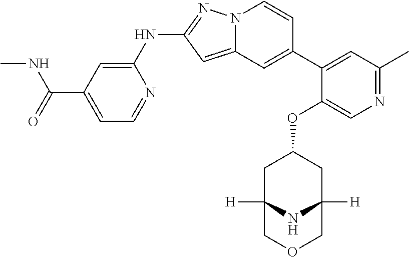

The title compound (20 mg, 6%) was prepared as described in Example 116 steps A-B where 2-bromo-N-methylisonicotinamide was used instead of 4-bromo-1-(difluoromethyl)-1H-pyrazole in step A. MS (ESI): mass calcd. for C₂₇H₂₉N₇O₃, 499.2. m/z found, 500.3 [M+H]⁺. ¹H NMR (400 MHz, DMSO-d6) δ 10.00 (s, 1H), 8.60 (q, J=4.4 Hz, 1H), 8.53 (d, J=7.2 Hz, 1H), 8.32 (d, J=5.2 Hz, 1H), 8.29 (s, 1H), 7.99 (s, 1H), 7.62 (s, 1H), 7.37 (s, 1H), 7.13-7.08 (m, 2H), 6.89 (s, 1H), 4.70-4.63 (m, 1H), 3.60-3.54 (m, 2H), 3.53-3.48 (m, 2H), 2.97-2.90 (m, 2H), 2.80 (d, J=4.4 Hz, 3H), 2.46 (s, 3H), 2.35-2.24 (m, 2H), 1.71-1.61 (m, 2H).

### Example 173: 6-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-N-methylnicotinamide

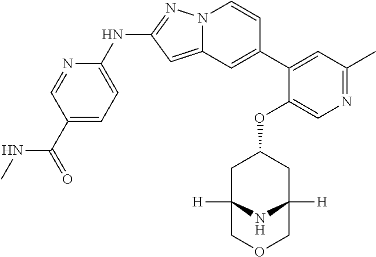

The title compound (43.6 mg, 34%) was prepared as described in Example 116 steps A-B where 6-bromo-N-methylnicotinamide was used instead of 4-bromo-1-(difluoromethyl)-1H-pyrazole in step A. MS (ESI): mass calcd. for C₂₇H₂₉N₇O₃, 499.2. m/z found, 500.3 [M+H]⁺. ¹H NMR (400 MHz, DMSO-d6) δ 10.19 (s, 1H), 8.71 (d, J=2.0 Hz, 1H), 8.55 (d, J=7.2 Hz, 1H), 8.37-8.31 (m, 1H), 8.30 (s, 1H), 8.07-8.00 (m, 2H), 7.38 (s, 1H), 7.35 (d, J=8.8 Hz, 1H), 7.12 (dd, J=1.2, 6.8 Hz, 1H), 6.86 (s, 1H), 4.75-4.62 (m, 1H), 3.66-3.59 (m, 2H), 3.59-3.51 (m, 2H), 3.08-2.99 (m, 2H), 2.79 (d, J=4.4 Hz, 3H), 2.46 (s, 3H), 2.38-2.28 (m, 2H), 1.79-1.68 (m, 2H).

### Example 174: N-(1-methyl-3-(trifluoromethyl)-1H-pyrazol-5-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine

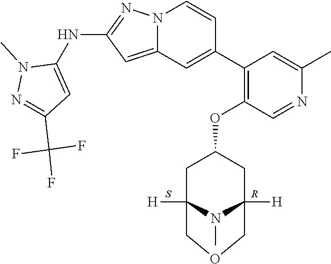

The title compound (10.5 mg, 3%) was prepared as described in Example 116, except 5-bromo-1-methyl-3-(trifluoromethyl)-1H-pyrazole was used instead of 4-bromo-1-(difluoromethyl)-1H-pyrazole in step A. MS (ESI): mass calcd. for C₂₆H₂₈N₇O₂F₃, 527.2. m/z found, 528.3 [M+H]⁺. ¹H NMR (400 MHz, DMSO-d6) δ 9.31 (s, 1H), 8.53 (d, J=7.2 Hz, 1H), 8.19 (s, 1H), 8.14 (s, 1H), 7.39 (s, 1H), 7.16 (dd, J=2.0, 7.2 Hz, 1H), 6.74 (s, 1H), 6.10 (s, 1H), 4.76-4.64 (m, 1H), 3.90-3.72 (m, 5H), 3.56-3.48 (m, 2H), 2.66-2.60 (m, 2H), 2.45 (s, 3H), 2.43-2.35 (m, 5H), 1.72-1.57 (m, 2H). ¹⁹F NMR (376 MHz, DMSO-d6) δ −60.9 (s, 3F).

### Example 175: N-(5-(5-(((1s,3s)-3-aminocyclobutyl)methoxy)-2-cyanopyridin-4-yl)pyrazolo[1,5-a]pyridin-2-yl)cyclopropanecarboxamide

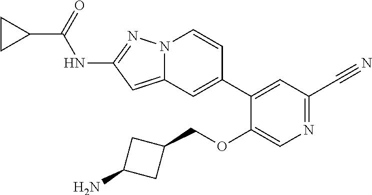 [SMILES: N#Cc1cc(-c2ccn3nc(NC(=O)C4CC4)cc3c2)c(OC[C@H]2C[C@@H](N)C2)cn1]

The title compound was prepared (70 mg, 6.5%) by the similar method of Example 8 by using tert-butyl ((cis)-3-(hydroxymethyl)cyclobutyl)carbamate instead of (2S,4R)-tert-butyl 4-hydroxy-2-(trifluoromethyl)piperidine-1-carboxylate. MS (ESI): mass calcd. for C₂₂H₂₂N₆O₂, 402.2. m/z found, 403.2 [M+H]⁺. ¹H NMR (400 MHz, DMSO-d6, t=80° C.) δ 10.78 (s, 1H), 8.60 (s, 1H), 8.56-8.51 (m, 1H), 8.08 (s, 1H), 7.90-7.86 (m, 1H), 7.06 (dd, J=2.0, 7.2 Hz, 1H), 6.91-6.88 (m, 1H), 4.36-4.12 (m, 2H), 3.31-3.13 (m, 1H), 2.33-2.19 (m, 3H), 1.98-1.91 (m, 1H), 1.54-1.42 (m, 2H), 0.88-0.83 (m, 2H), 0.83-0.77 (m, 2H).

### Example 176: cis-N-[5-[2-cyano-5-[[3-(2-pyridylamino)cyclobutyl]methoxy]-4-pyridyl]pyrazolo[1,5-a]pyridin-2-yl]cyclopropanecarboxamide

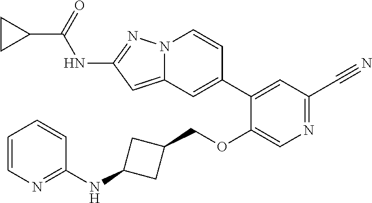 [SMILES: N#Cc1cc(-c2ccn3nc(NC(=O)C4CC4)cc3c2)c(OC[C@H]2C[C@@H](Nc3ccccn3)C2)cn1]

The title compound was prepared (3 mg, 1.2%) using the method described in Example 260 and using Example 175 instead of 1-((4-(2-aminopyrazolo[1,5-a]pyridin-5-yl)-6-methylpyridin-3-yl)oxy)-2-methylpropan-2-ol and using 2-bromopyridine instead of 6-chloroimidazo[1,2-b]pyridazine. MS (ESI): mass calcd. for C₂₇H₂₅N₇O₂, 479.2. m/z found, 480.3 [M+H]⁺. ¹H NMR (400 MHz, DMSO-d6) δ 11.14 (s, 1H), 8.66 (s, 1H), 8.61 (d, J=7.2 Hz, 1H), 8.19 (s, 1H), 7.96-7.90 (m, 2H), 7.38-7.31 (m, 1H), 7.13 (dd, J=2.0, 7.2 Hz, 1H), 6.94 (s, 1H), 6.61 (d, J=6.8 Hz, 1H), 6.49-6.43 (m, 1H), 6.39 (d, J=8.8 Hz, 1H), 4.30 (d, J=4.8 Hz, 2H), 4.24-4.11 (m, 1H), 2.47-2.39 (m, 3H), 1.98-1.89 (m, 1H), 1.82-1.69 (m, 2H), 0.89-0.77 (m, 4H)

### Example 177: cis-N-[5-[2-cyano-5-[[3-[(1-methylpyrazol-3-yl)amino]cyclobutyl]methoxy]-4-pyridyl]pyrazolo[1,5-a]pyridin-2-yl]cyclopropanecarboxamide

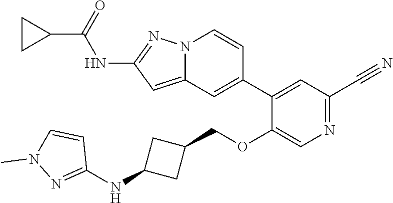 [SMILES: Cn1ccc(N[C@H]2C[C@@H](COc3cnc(C#N)cc3-c3ccn4nc(NC(=O)C5CC5)cc4c3)C2)n1]

The title compound was prepared (55 mg, 25%) using the method described in Example 260 and using Example 175 instead of 1-((4-(2-aminopyrazolo[1,5-a]pyridin-5-yl)-6-methylpyridin-3-yl)oxy)-2-methylpropan-2-ol and using 3-bromo-1-methyl-1H-pyrazole instead of 6-chloroimidazo[1,2-b]pyridazine. MS (ESI): mass calcd. for C₂₆H₂₆N₈O₂, 482.2. m/z found, 483.3 [M+H]⁺. ¹H NMR (400 MHz, DMSO-d6) δ 11.14 (s, 1H), 8.65 (s, 1H), 8.63 (d, J=7.2 Hz, 1H), 8.19 (s, 1H), 7.92 (d, J=1.2 Hz, 1H), 7.27 (d, J=2.0 Hz, 1H), 7.12 (dd, J=2.0, 7.2 Hz, 1H), 6.93 (s, 1H), 5.35 (d, J=2.4 Hz, 1H), 5.15 (d, J=8.4 Hz, 1H), 4.28 (d, J=5.2 Hz, 2H), 3.81-3.70 (m, 1H), 3.58 (s, 3H), 2.44-2.34 (m, 3H), 1.97-1.89 (m, 1H), 1.73-1.61 (m, 2H), 0.87-0.78 (m, 4H).

### Example 178: rac-N-[5-[2-cyano-5-[[4-hydroxy-4-(trifluoromethyl)cyclohexyl]methoxy]-4-pyridyl]pyrazolo[1,5-a]pyridin-2-yl]cyclopropanecarboxamide

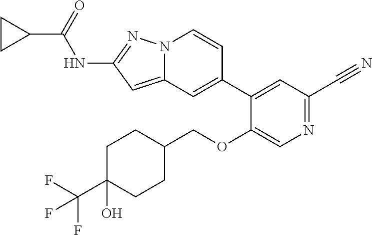 [SMILES: N#Cc1cc(-c2ccn3nc(NC(=O)C4CC4)cc3c2)c(OCC2CCC(O)(C(F)(F)F)CC2)cn1]

Step A: N-(5-(2-cyano-5-((4-oxocyclohexyl)methoxy)pyridin-4-yl)pyrazolo[1,5-a]pyridin-2-yl)cyclopropanecarboxamide. The title compound was prepared (297 mg, 33%) by the similar method of Example 8 by using 4-(hydroxymethyl)cyclohexanone instead of (2S,4R)-tert-butyl 4-hydroxy-2-(trifluoromethyl)piperidine-1-carboxylate. ¹H NMR (400 MHz, DMSO-d₆) δ 11.10 (s, 1H), 8.66 (s, 1H), 8.59 (d, J=7.2 Hz, 1H), 8.15 (s, 1H), 7.90-7.84 (m, 1H), 7.06 (dd, J=2.0, 7.2 Hz, 1H), 6.89 (s, 1H), 4.24 (d, J=6.8 Hz, 2H), 2.43-2.24 (m, 3H), 2.23-2.12 (m, 2H), 2.06-1.97 (m, 2H), 1.95-1.85 (m, 1H), 1.55-1.38 (m, 2H), 0.86-0.75 (m, 4H).

Step B: rac-N-[5-[2-cyano-5-[[4-hydroxy-4-(trifluoromethyl)cyclohexyl]methoxy]-4-pyridyl]pyrazolo[1,5-a]pyridin-2-yl]cyclopropanecarboxamide. Trimethyl(trifluoromethyl)silane (120 mg, 0.84 mmol) was added to a solution consisting of N-(5-(2-cyano-5-((4-oxocyclohexyl)methoxy)pyridin-4-yl)pyrazolo[1,5-a]pyridin-2-yl)cyclopropanecarboxamide (2900 mg, 0.68 mmol) and THF (10 mL) cooled to 0° C. Tetrabutylammonium fluoride (3.4 mL, 3.4 mmol was then added and the vial was removed from the ice bath and contents were allowed to warm to 23° C. over 2 h. Contents were then treated with aqueous 1N HCl (5 mL) and stirred at 23° C. for 16 h. The reactant mixture was poured into water (40 mL) and extracted with ethyl acetate (50 mL×3). The organic extracts were washed with brine (20 mL×3), dried over anhydrous Na₂SO₄, filtered, and concentrated to dryness under reduced pressure to obtain the crude product, which was purified by FCC (eluent: petroleum ether:ethylacetate=1:0 to 0:1) to afford the product which was purified further by SFC over DAICEL DAICEL CHIRALPAK AD 250 mm×30 mm, 10 μm (eluent: 50% to 50% (v/v) 0.1% NH₃ H₂O IPA). The pure fractions were collected and the volatiles were removed under reduced pressure. The product was suspended in water (10 mL), the mixture frozen using dry ice/acetone, and then lyophilized to dryness to afford the title compound (34.70 mg, 10%) as a white solid. MS (ESI): mass calcd. for C₂₅H₂₄F₃N₅O₃, 499.2. m/z found, 500.2 [M+H]⁺. ¹H NMR (400 MHz, DMSO-d₆) δ 11.15 (s, 1H), 8.73 (s, 1H), 8.62 (d, J=7.2 Hz, 1H), 8.17 (s, 1H), 7.90 (d, J=1.2 Hz, 1H), 7.08 (dd, J=2.0, 7.2 Hz, 1H), 6.91 (s, 1H), 5.73 (br. s., 1H), 4.29 (d, J=7.6 Hz, 2H), 2.14-2.04 (m, 1H), 1.98-1.89 (m, 1H), 1.83-1.71 (m, 4H), 1.57-1.48 (m, 4H), 0.86-0.79 (m, 4H). ¹⁹F NMR (376 MHz, DMSO-d₆) −81.50 (s, 3F).

### Example 179: cis-N-(5-(5-(((1*S,3*R)-3-hydroxycyclopentyl)oxy)-2-methylpyridin-4-yl)pyrazolo[1,5-a]pyridin-2-yl)cyclopropanecarboxamide

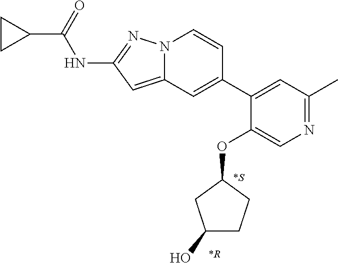 [SMILES: Cc1cc(-c2ccn3nc(NC(=O)C4CC4)cc3c2)c(O[C@H]2CC[C@@H](O)C2)cn1]

The title compound was prepared as the racemate (140 mg, 40%) by the similar method of Example 8 by using (1R,3S)-cyclopentane-1,3-diol instead of tert-butyl (2S,4R)-4-hydroxy-2-(trifluoromethyl)piperidine-1-carboxylate and using Intermediate 6 instead of Intermediate 4. N-(5-(5-(((1S,3R)-3-hydroxycyclopentyl)oxy)-2-methylpyridin-4-yl)pyrazolo[1,5-a]pyridin-2-yl)cyclopropanecarboxamide (140 mg, 0.319 mmol) was purified by supercritical fluid chromatography over DAICEL CHIRALPAK IC (250 mm×30 mm×10 um) (eluent: supercritical CO₂ in EtOH (0.1% v/v ammonia). The pure fractions were collected and the volatiles were removed under vacuum. The resulting product was lyophilized to dryness to remove the solvent residue completely. The first eluting fraction (53 mg) was purified further by supercritical fluid chromatography over DAICEL CHIRALPAK AD (250 mm×30 mm×10 um) (eluent: supercritical CO₂ in EtOH (0.1% v/v ammonia) 50/50, v/v). The pure fractions were collected and the volatiles were removed under vacuum. The resulting product was lyophilized to dryness to remove the solvent residue completely. The title compound (39.5 mg) was obtained as a yellow solid. MS (ESI): mass calcd. for C₂₂H₂₄N₄O₃, 392.2. m/z found, 393.2 [M+H]⁺. ¹H NMR (400 MHz, DMSO-d6) δ 11.10 (s, 1H), 8.54 (d, J=7.2 Hz, 1H), 8.25 (s, 1H), 7.92 (d, J=1.2 Hz, 1H), 7.35 (s, 1H), 7.12 (dd, J=2.0, 7.2 Hz, 1H), 6.88 (s, 1H), 4.92-4.83 (m, 1H), 4.69 (d, J=4.0 Hz, 1H), 4.19-4.08 (m, 1H), 2.45 (s, 3H), 2.32-2.22 (m, 1H), 1.99-1.89 (m, 2H), 1.88-1.71 (m, 2H), 1.69-1.58 (m, 2H), 0.87-0.77 (m, 4H).

### Example 180: cis-N-(5-(5-(((1*R,3*S)-3-hydroxycyclopentyl)oxy)-2-methylpyridin-4-yl)pyrazolo[1,5-a]pyridin-2-yl)cyclopropanecarboxamide

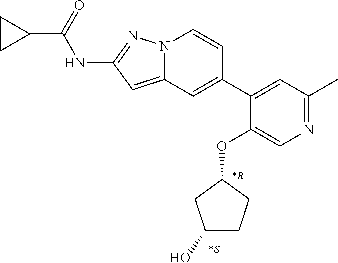 [SMILES: Cc1cc(-c2ccn3nc(NC(=O)C4CC4)cc3c2)c(O[C@@H]2CC[C@H](O)C2)cn1]

The second eluting fraction from Example 179 (55 mg) was purified by supercritical fluid chromatography over DAICEL CHIRALPAK AS-H (250 mm×30 mm×5 um) (eluent: supercritical CO₂ in EtOH (0.1% v/v ammonia) 50/50, v/v). The pure fractions were collected and the volatiles were removed under vacuum. The resulting product was lyophilized to dryness to remove the solvent residue completely. The title compound (42 mg) was obtained as a yellow solid. MS (ESI): mass calcd. for C₂₂H₂₄N₄O₃, 392.2. m/z found, 393.2 [M+H]⁺. ¹H NMR (400 MHz, DMSO-d6) δ 11.09 (s, 1H), 8.54 (d, J=7.2 Hz, 1H), 8.25 (s, 1H), 7.92 (d, J=1.2 Hz, 1H), 7.35 (s, 1H), 7.12 (dd, J=2.0, 7.2 Hz, 1H), 6.88 (s, 1H), 4.94-4.82 (m, 1H), 4.68 (d, J=3.6 Hz, 1H), 4.22-4.03 (m, 1H), 2.45 (s, 3H), 2.32-2.23 (m, 1H), 2.00-1.89 (m, 2H), 1.88-1.71 (m, 2H), 1.70-1.58 (m, 2H), 0.87-0.74 (m, 4H).

### Example 181: trans-4-[[6-methyl-4-[2-[(1-methylpyrazol-3-yl)amino]pyrazolo[1,5-a]pyridin-5-yl]-3-pyridyl]oxy]cyclohexanol

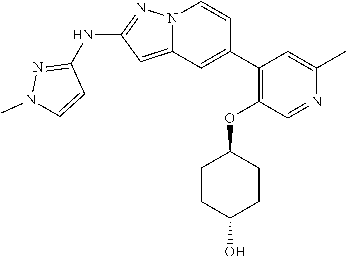

The title compound was prepared (33 mg, 30%) using the method described in Example 260 and using Intermediate 12 instead of 1-((4-(2-aminopyrazolo[1,5-a]pyridin-5-yl)-6-methylpyridin-3-yl)oxy)-2-methylpropan-2-ol and using 3-bromo-1-methyl-1H-pyrazole instead of 6-chloroimidazo[1,2-b]pyridazine. MS (ESI): mass calcd. for C₂₃H₂₆N₆O₂, 418.2. m/z found, 419.3 [M+H]⁺. ¹H NMR (400 MHz, DMSO-d6) δ 9.05 (s, 1H), 8.43 (d, J=7.1 Hz, 1H), 8.33 (s, 1H), 7.66-7.61 (m, 1H), 7.49 (d, J=2.2 Hz, 1H), 7.31 (s, 1H), 6.85 (dd, J=7.1, 2.0 Hz, 1H), 6.41 (s, 1H), 6.05 (d, J=2.2 Hz, 1H), 4.50 (d, J=3.7 Hz, 1H), 4.39 (dt, J=8.9, 4.9 Hz, 1H), 3.73 (s, 3H), 3.57-3.43 (m, 1H), 2.45 (s, 3H), 2.01-1.91 (m, 2H), 1.74 (d, J=13.1 Hz, 2H), 1.47-1.34 (m, 2H), 1.33-1.21 (m, 2H).

### Example 182: trans-4-[[6-methyl-4-[2-(2-pyridylamino)pyrazolo[1,5-a]pyridin-5-yl]-3-pyridyl]oxy]cyclohexanol

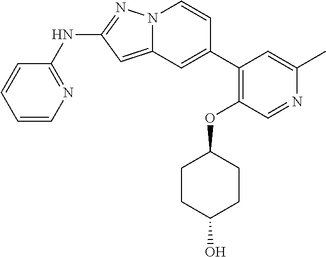 [SMILES: Cc1cc(-c2ccn3nc(Nc4ccccn4)cc3c2)c(O[C@H]2CC[C@H](O)CC2)cn1]

The title compound was prepared (65 mg, 26%) using the method described in Example 260 and using Intermediate 12 instead of 1-((4-(2-aminopyrazolo[1,5-a]pyridin-5-yl)-6-methylpyridin-3-yl)oxy)-2-methylpropan-2-ol and using 2-bromopyridine instead of 6-chloroimidazo[1,2-b]pyridazine and using Pd₂dba₃/Xantphos instead of t-BuBrettPhos-Pd-G3. MS (ESI): mass calcd. for C₂₄H₂₅N₅O₂, 415.2. m/z found, 416.2 [M+H]⁺. ¹H NMR (400 MHz, DMSO-d6) δ 9.77 (s, 1H), 8.52 (dt, J=7.2, 0.9 Hz, 1H), 8.35 (s, 1H), 8.21 (ddd, J=5.0, 2.0, 0.8 Hz, 1H), 7.74 (dd, J=2.0, 0.9 Hz, 1H), 7.62 (ddd, J=8.5, 7.2, 2.0 Hz, 1H), 7.35-7.26 (m, 2H), 6.94 (dd, J=7.2, 2.0 Hz, 1H), 6.86 (d, J=0.9 Hz, 1H), 6.80 (ddd, J=7.1, 4.9, 1.0 Hz, 1H), 4.50 (d, J=4.0 Hz, 1H), 4.41 (dt, J=8.8, 4.7 Hz, 1H), 3.49 (dq, J=8.7, 4.4 Hz, 1H), 2.45 (s, 3H), 2.03-1.91 (m, 2H), 1.74 (d, J=12.4 Hz, 2H), 1.49-1.35 (m, 2H), 1.34-1.21 (m, 2H).

### Example 183: trans-N-(5-(5-(((1*S,3*S)-3-hydroxycyclopentyl)oxy)-2-methylpyridin-4-yl)pyrazolo[1,5-a]pyridin-2-yl)cyclopropanecarboxamide

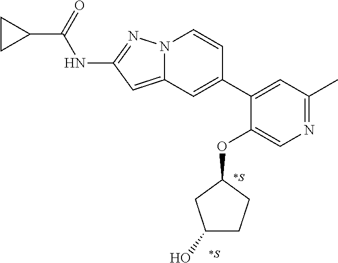 [SMILES: Cc1cc(-c2ccn3nc(NC(=O)C4CC4)cc3c2)c(O[C@H]2CC[C@H](O)C2)cn1]

The title compound was prepared as the racemate (95 mg, 24%) by the similar method of Example 8 by using (1S,3S)-cyclopentane-1,3-diol instead of (2S,4R)-tert-butyl 4-hydroxy-2-(trifluoromethyl)piperidine-1-carboxylate and using Intermediate 6 instead of Intermediate 4. N-(5-(5-(((1S,3 S)-3-hydroxycyclopentyl)oxy)-2-methylpyridin-4-yl)pyrazolo[1,5-a]pyridin-2-yl)cyclopropanecarboxamide (95 mg, 0.24 mmol) was purified by supercritical fluid chromatography over DAICEL CHIRALPAK AD (250 mm×30 mm×10 um) (eluent: supercritical CO₂ in EtOH (0.1% v/v ammonia) 50/50, v/v). The pure fractions were collected and the volatiles were removed under vacuum. The resulting product was lyophilized to dryness to remove the solvent residue completely to afford the title compound (16.8 mg) as a yellow solid. MS (ESI): mass calcd. for C₂₂H₂₄N₄O₃, 392.2. m/z found, 393.2 [M+H]⁺. ¹H NMR (400 MHz, DMSO-d6) δ 11.10 (s, 1H), 8.56 (d, J=7.2 Hz, 1H), 8.27 (s, 1H), 7.77 (s, 1H), 7.34 (s, 1H), 7.00 (dd, J=1.6, 7.2 Hz, 1H), 6.89 (s, 1H), 5.05-4.94 (m, 1H), 4.62 (d, J=2.8 Hz, 1H), 4.25-4.14 (m, 1H), 2.45 (s, 3H), 2.19-2.06 (m, 1H), 2.00-1.87 (m, 3H), 1.86-1.76 (m, 1H), 1.72-1.62 (m, 1H), 1.54-1.43 (m, 1H), 0.90-0.77 (m, 4H).

### Example 184: cis-4-[[6-methyl-4-[2-[(1-methylpyrazol-3-yl)amino]pyrazolo[1,5-a]pyridin-5-yl]-3-pyridyl]oxy]cyclohexanol

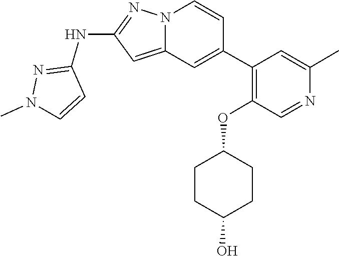

The title compound was prepared (8.3 mg, 17%) using the method described in Example 260 and using Intermediate 12 instead of 1-((4-(2-aminopyrazolo[1,5-a]pyridin-5-yl)-6-methylpyridin-3-yl)oxy)-2-methylpropan-2-ol and using 3-bromo-1-methyl-1H-pyrazole instead of 6-chloroimidazo[1,2-b]pyridazine. MS (ESI): mass calcd. for C₂₃H₂₆N₆O₂, 418.2. m/z found, 419.3 [M+H]⁺. ¹H NMR (400 MHz, DMSO-d6) δ 9.05 (s, 1H), 8.44 (dt, J=7.2, 0.9 Hz, 1H), 8.31 (s, 1H), 7.67 (dd, J=2.0, 0.9 Hz, 1H), 7.48 (d, J=2.2 Hz, 1H), 7.32 (s, 1H), 6.89 (dd, J=7.2, 2.0 Hz, 1H), 6.41 (d, J=0.9 Hz, 1H), 6.06 (d, J=2.2 Hz, 1H), 4.48 (s, 1H), 4.44 (d, J=3.8 Hz, 1H), 3.73 (s, 3H), 2.45 (s, 3H), 1.88-1.77 (m, 2H), 1.65-1.40 (m, 6H).

### Example 185: trans-N-[5-[5-(4-hydroxycyclohexoxy)-2-prop-1-ynyl-4-pyridyl]pyrazolo[1,5-a]pyridin-2-yl]cyclopropanecarboxamide

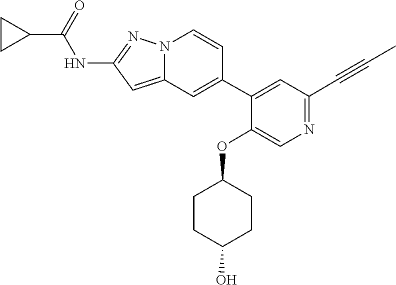 [SMILES: CC#Cc1cc(-c2ccn3nc(NC(=O)C4CC4)cc3c2)c(O[C@H]2CC[C@H](O)CC2)cn1]

The title compound was prepared (10 mg, 6.5%) by the similar method of Example 1 using Intermediate 11 instead of N-(5-(5-chloro-2-cyanopyridin-4-yl)pyrazolo[1,5-a]pyridin-2-yl)cyclopropanecarboxamide and trans-(1r,4r)-cyclohexane-1,4-diol instead of (R)-(1-methylpyrrolidin-3-yl)methanol. MS (ESI): mass calcd. for C₂₅H₂₆N₄O₃, 430.2. m/z found, 431.2 [M+H]⁺. ¹H NMR (400 MHz, DMSO-d6) δ 11.08 (s, 1H), 8.54 (d, J=7.3 Hz, 1H), 8.45 (s, 1H), 7.82 (dd, J=2.0, 0.9 Hz, 1H), 7.51 (s, 1H), 7.03 (dd, J=7.2, 2.0 Hz, 1H), 6.93-6.84 (m, 1H), 4.57 (ddd, J=19.6, 10.0, 4.1 Hz, 2H), 2.06 (s, 3H), 2.04-1.95 (m, 3H), 1.79-1.67 (m, 2H), 1.50-1.38 (m, 2H), 1.38-1.26 (m, 2H), 0.88-0.75 (m, 4H).

### Example 186: N-[5-[2-methyl-5-(4-oxocyclohexoxy)-4-pyridyl]pyrazolo[1,5-a]pyridin-2-yl]cyclopropanecarboxamide

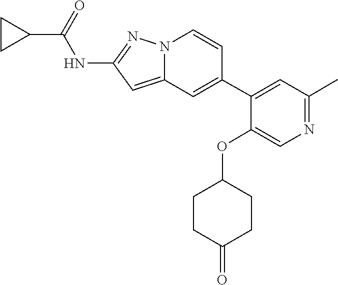 [SMILES: Cc1cc(-c2ccn3nc(NC(=O)C4CC4)cc3c2)c(OC2CCC(=O)CC2)cn1]

Step A: N-(5-(5-((1,4-dioxaspiro[4.5]decan-8-yl)oxy)-2-methylpyridin-4-yl)pyrazolo[1,5-a]pyridin-2-yl)cyclopropanecarboxamide. The title compound was prepared (1700 mg) by the similar method of Example 1 using Intermediate 6 instead of N-(5-(5-chloro-2-cyanopyridin-4-yl)pyrazolo[1,5-a]pyridin-2-yl)cyclopropanecarboxamide and 1,4-dioxaspiro[4.5]decan-8-ol instead of (R)-(1-methylpyrrolidin-3-yl)methanol. ¹H NMR (400 MHz, DMSO-d6) δ 11.11 (s, 1H), 8.57 (d, J=7.2 Hz, 1H), 8.37 (s, 1H), 7.81 (s, 1H), 7.35 (s, 1H), 7.03 (dd, J=1.8, 7.2 Hz, 1H), 6.88 (s, 1H), 4.63-4.57 (m, 1H), 3.91-3.71 (m, 4H), 2.45 (s, 3H), 1.95-1.88 (m, 1H), 1.85-1.68 (m, 4H), 1.62-1.44 (m, 4H), 0.87-0.75 (m, 4H).

Step B: N-[5-[2-methyl-5-(4-oxocyclohexoxy)-4-pyridyl]pyrazolo[1,5-a]pyridin-2-yl]cyclopropanecarboxamide. To a mixture solution consisting of N-(5-(5-(1,4-dioxaspiro[4.5]decan-8-yloxy)-2-methylpyridin-4-yl)pyrazolo[1,5-a]pyridin-2-yl)cyclopropanecarboxamide (100 mg, 0.223 mmol) and CH₂Cl₂ (5 mL) was added FeCl₃·6H₂O (362 mg, 1.34 mmol) at 25° C. The reaction mixture was stirred at room-temperature for 12 hrs. The reaction mixture was diluted with 50 mL EtOAc, adjusted to pH 8 with aqueous NaHCO₃ and filtered through a pad of Celite® and the pad was washed with EtOAc (10 mL×2). The organic layer was separated and concentrated to dryness under reduced pressure to give the crude product. The crude product was purified by preparative HPLC using a Xtimate C18 250×50 mm×10 μm column (eluent: 30% to 60% (v/v) CH₃CN and H₂O with 0.04% NH₃ and 10 mM NH₄HCO₃) to afford pure product. The product was suspended in water (10 mL), the mixture frozen using dry ice/ethanol, and then lyophilized to dryness to afford the title compound (50 mg, 55%) as a white solid. MS (ESI): mass calcd. for C₂₃H₂₄N₄O₃, 404.2. m/z found, 405.2 [M+H]⁺. ¹H NMR (400 MHz, DMSO-d6) δ 11.10 (s, 1H), 8.57 (d, J=7.2 Hz, 1H), 8.47 (s, 1H), 7.83 (s, 1H), 7.38 (s, 1H), 7.07 (dd, J=2.0, 7.2 Hz, 1H), 6.90 (s, 1H), 4.87-4.82 (m, 1H), 2.48 (s, 3H), 2.36-2.27 (m, 2H), 2.26-2.17 (m, 2H), 2.13-1.99 (m, 4H), 1.98-1.85 (m, 1H), 0.90-0.75 (m, 4H).

### Example 187: trans-4-[[4-[2-[(1-methylpyrazol-3-yl)amino]pyrazolo[1,5-a]pyridin-5-yl]-6-prop-1-ynyl-3-pyridyl]oxy]cyclohexanol

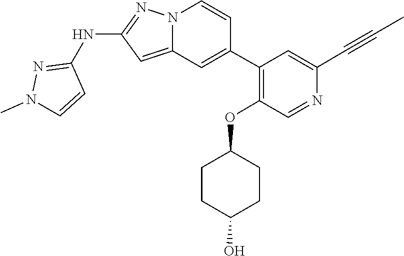 [SMILES: CC#Cc1cc(-c2ccn3nc(Nc4ccn(C)n4)cc3c2)c(O[C@H]2CC[C@H](O)CC2)cn1]

The title compound was prepared (8.1 mg, 6.3%) by the similar method of Example 1 using trans-(1r,4r)-cyclohexane-1,4-diol instead of (R)-(1-methylpyrrolidin-3-yl)methanol and Intermediate 19 instead of N-(5-(5-chloro-2-cyanopyridin-4-yl)pyrazolo[1,5-a]pyridin-2-yl)cyclopropanecarboxamide. MS (ESI): mass calcd. for C₂₅H₂₆N₆O₂, 442.2. m/z found, 443.1 [M+H]⁺. ¹H NMR (400 MHz, DMSO-d6) δ 9.06 (s, 1H), 8.42 (d, J=7.8 Hz, 2H), 7.67 (dd, J=1.9, 0.9 Hz, 1H), 7.48 (d, J=4.2 Hz, 2H), 6.86 (dd, J=7.2, 2.0 Hz, 1H), 6.42 (d, J=0.8 Hz, 1H), 6.04 (d, J=2.2 Hz, 1H), 4.62-4.48 (m, 2H), 3.73 (s, 3H), 3.51 (s, 1H), 2.06 (s, 2H), 2.04-1.95 (m, 2H), 1.74 (d, J=12.2 Hz, 2H), 1.44 (q, J=9.6 Hz, 2H), 1.32 (q, J=9.0 Hz, 2H).

### Example 188: rac-N-[5-[5-[(4,4-difluoro-1-hydroxy-cyclohexyl)methoxy]-2-methyl-4-pyridyl]pyrazolo[1,5-a]pyridin-2-yl]cyclopropanecarboxamide

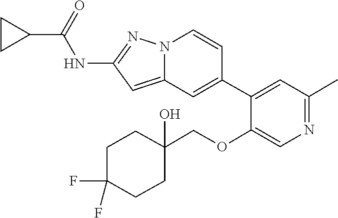 [SMILES: Cc1cc(-c2ccn3nc(NC(=O)C4CC4)cc3c2)c(OCC2(O)CCC(F)(F)CC2)cn1]

A mixture of Intermediate 7 (100 mg, 0.324 mmol), 4,4-difluoro-(1-hydroxycyclohexyl)methyl 4-methylbenzenesulfonate (200 mg, 0.624 mmol), Cs₂CO₃ (317 mg, 0.973 mmol) and DMF (5 mL) was stirred at 70° C. for 16 h. The reactant mixture was purified by preparative HPLC using a Boston Green ODS 150×30 mm×5 μm column (eluent: 30% to 60% (v/v) CH₃CN and H₂O with 0.04% NH₃) to afford pure product. The product was suspended in water (10 mL), the mixture frozen using dry ice/acetone, and then lyophilized to dryness to afford the title compound (12.1 mg, 8%) as a yellow solid. MS (ESI): mass calcd. for C₂₄H₂₆F₂N₄O₃, 456.2. m/z found, 457.2 [M+H]⁺. ¹H NMR (400 MHz, DMSO-d6) δ 11.08 (s, 1H), 8.53 (d, J=7.2 Hz, 1H), 8.33 (s, 1H), 7.88 (d, J=1.2 Hz, 1H), 7.37 (s, 1H), 7.09 (dd, J=2.0, 7.2 Hz, 1H), 6.87 (s, 1H), 4.85 (s, 1H), 3.97 (s, 2H), 2.46 (s, 3H), 2.14-1.80 (m, 5H), 1.74-1.63 (m, 4H), 0.89-0.77 (m, 4H). ¹⁹F NMR (376 MHz, DMSO-d6) δ −89.91 (d, J=234.2 Hz, 1F), −101.75 (d, J=231.6 Hz, 1F).

### Example 189: (*R)—N-(5-(2-methyl-5-(3,3,3-trifluoro-2-hydroxy-2-methylpropoxy)pyridin-4-yl)pyrazolo[1,5-a]pyridin-2-yl)cyclopropanecarboxamide

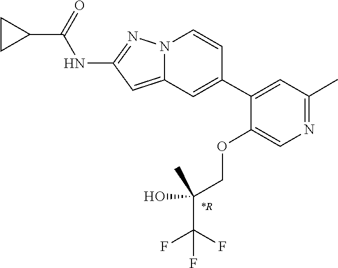 [SMILES: Cc1cc(-c2ccn3nc(NC(=O)C4CC4)cc3c2)c(OC[C@@](C)(O)C(F)(F)F)cn1]

The title compound was prepared as described in Example 193, Step B, and using 2-methyl-2-(trifluoromethyl)oxirane instead of 2-cyclopropyl-2-methyloxirane. N-(5-(2-methyl-5-(3,3,3-trifluoro-2-hydroxy-2-methylpropoxy)pyridin-4-yl)pyrazolo[1,5-a]pyridin-2-yl)cyclopropanecarboxamide (40 mg, 0.092 mmol) was further purified by SFC over Phenomenex-Cellulose-2 (250 mm×30 mm, 10 um), (eluent: 45% to 45% (v/v) supercritical CO₂ in EtOH and H₂O with 0.1% NH₃). The pure fractions were collected and the volatiles were removed under reduced pressure. The products were suspended in water (10 mL), the mixture frozen using dry ice/ethanol, and then lyophilized to dryness to afford the title compound (13.9 mg, 33%). MS (ESI): mass calcd. for C₂₁H₂₁F₃N₄O₃, 434.2. m/z found, 435.0 [M+H]⁺. ¹H NMR (400 MHz, CDCl₃) δ 8.47 (s, 1H), 8.26 (s, 1H), 8.19 (d, J=7.2 Hz, 1H), 7.60 (d, J=1.2 Hz, 1H), 7.19 (s, 1H), 6.92 (s, 1H), 6.84 (dd, J=1.6, 7.2 Hz, 1H), 4.32 (d, J=9.6 Hz, 1H), 4.10 (d, J=9.6 Hz, 1H), 2.56 (s, 3H), 1.48 (s, 3H), 1.41-1.37 (m, 1H), 1.41 (d, J=5.2 Hz, 1H), 1.10-1.02 (m, 2H), 0.85-0.75 (m, 2H). ¹⁹F NMR (376 MHz, CDCl₃) δ −80.52 (s, 3F).

### Example 190: (*S)—N-(5-(2-methyl-5-(3,3,3-trifluoro-2-hydroxy-2-methylpropoxy)pyridin-4-yl)pyrazolo[1,5-a]pyridin-2-yl)cyclopropanecarboxamide

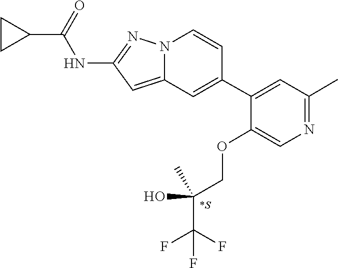 [SMILES: Cc1cc(-c2ccn3nc(NC(=O)C4CC4)cc3c2)c(OC[C@](C)(O)C(F)(F)F)cn1]

The title compound (12.8 mg, 30%) was prepared as described in Example 189 and is the other isomer from the SFC purification. MS (ESI): mass calcd. for C₂₁H₂₁F₃N₄O₃, 434.2. m/z found, 435.1 [M+H]⁺. ¹H NMR (400 MHz, CDCl₃) δ 8.45 (s, 1H), 8.32 (s, 1H), 8.21 (d, J=7.6 Hz, 1H), 7.63-7.57 (m, 1H), 7.23 (s, 1H), 6.94 (s, 1H), 6.86 (dd, J=1.6, 7.2 Hz, 1H), 4.32 (d, J=10.0 Hz, 1H), 4.12 (d, J=9.6 Hz, 1H), 2.59 (s, 3H), 1.47 (s, 3H), 1.45-1.40 (m, 1H), 1.11-1.03 (m, 2H), 0.87-0.79 (m, 2H). ¹⁹F NMR (376 MHz, CDCl₃) δ −80.50 (s, 3F).

### Example 191: N-(5-(5-(((1r,4r)-4-hydroxy-4-(trifluoromethyl)cyclohexyl)oxy)-2-methylpyridin-4-yl)pyrazolo[1,5-a]pyridin-2-yl)cyclopropanecarboxamide

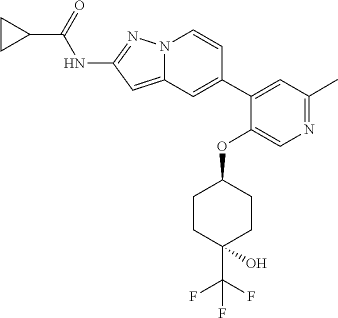 [SMILES: Cc1cc(-c2ccn3nc(NC(=O)C4CC4)cc3c2)c(O[C@H]2CC[C@@](O)(C(F)(F)F)CC2)cn1]

Step A: Trimethyl((8-(trifluoromethyl)-1,4-dioxaspiro[4.5]decan-8-yl)oxy)silane. To a mixture of 1,4-dioxaspiro[4.5]decan-8-one (3.00 g, 19.2 mmol) and CsF (5.84 g, 38.4 mmol) in THF (30.0 mL) was added a solution of trimethyl(trifluoromethyl)silane (4.10 g, 28.8 mmol) and THF (15.0 mL) at 0° C. The reaction mixture was allowed to warm to 15° C. and the reaction mixture was stirred at that temperature for 14 h. The reaction mixture was filtered, and the filtrate concentrated under reduced pressure to afford the crude, which was purified by FCC (petroleum ether:ethyl acetate=0 to 10:1) to afford the title compound (4.3 g, 75% yield) as a colorless oil.

Step B: 4-Hydroxy-4-(trifluoromethyl)cyclohexan-1-one. To a mixture of trimethyl((8-(trifluoromethyl)-1,4-dioxaspiro[4.5]decan-8-yl)oxy)silane (1.00 g, 3.35 mmol) and THF (5 mL) was added HCl (5 mL, 5 mmol, 1 M) at 10-15° C. The reaction mixture was stirred at 10-15° C. for 18 hrs. The reaction mixture was extracted with ethyl acetate (3×10 mL). The combined organic layers were dried over sodium sulfate, filtered and concentrated under reduced pressure to afford the crude, which was purified by FCC (petroleum ether:ethyl acetate=0 to 3:1) to give the title compound (0.6 g, 98% yield) as a colorless oil.

Step C: 1-(Trifluoromethyl)cyclohexane-1,4-diol. To a mixture of 4-hydroxy-4-(trifluoromethyl)cyclohexan-1-one (600 mg, 3.29 mmol) and MeOH (10.0 mL) was added NaBH₄ (250 mg, 6.59 mmol) at 10-15° C. The reaction mixture was stirred at 10-15° C. for 1 h. The reaction mixture was quenched with water (10 mL) and concentrated under reduced pressure. The mixture was extracted with ethyl acetate (3×10 mL). The combined organic layers were dried over sodium sulfate, filtered and concentrated under reduced pressure to afford the crude, which was purified by FCC (petroleum ether:ethyl acetate=1:0 to 0:1) to give the title compound (0.4 g, 66% yield) as a white solid.

Step D: 4-Hydroxy-4-(trifluoromethyl)cyclohexyl 4-methylbenzenesulfonate. To a mixture consisting of 1-(trifluoromethyl)cyclohexane-1,4-diol (400 mg, 2.17 mmol) and DCM (5 mL) was added TEA (440 mg, 4.34 mmol) and a solution consisting of p-toluenesulfonyl chloride (414 mg, 2.17 mmol) in DCM (5 mL) at 25° C. The mixture was stirred at 25° C. for 13 hrs. N,N-dimethylpyridin-4-amine (440 mg, 4.34 mmol) was added and the mixture was stirred at 10-15° C. for 4 hrs. The reaction mixture was purified by FCC (petroleum ether:ethyl acetate=1:0 to 3:2) to afford the title compound (120 mg, 16% yield) as a white solid.

Step E: N-(5-(5-(((1r,4r)-4-hydroxy-4-(trifluoromethyl)cyclohexyl)oxy)-2-methylpyridin-4-yl)pyrazolo[1,5-a]pyridin-2-yl)cyclopropanecarboxamide. To a mixture of N-(5-(5-hydroxy-2-methylpyridin-4-yl)pyrazolo[1,5-a]pyridin-2-yl)cyclopropanecarboxamide (Intermediate 7, 100 mg, 0.324 mmol) and 4-hydroxy-4-(trifluoromethyl)cyclohexyl 4-methylbenzenesulfonate (110 mg, 0.324 mmol) in DMF (5.0 mL) was added cesium carbonate (317 mg, 0.973 mmol). The reaction mixture was stirred at 75° C. for 2 hrs, then worked-up by dilution with ethyl acetate (20 mL), filtration, and concentration under reduced pressure. The crude product was purified by preparative HPLC using a Boston Green ODS 150×30 mm×5 μm column (eluent: 35% to 65 (v/v) CH₃CN and H₂O with 0.04% NH₃ and 10 mM NH₄HCO₃) and lyophilized to afford the title compound (9.4 mg, 6.1%) as a white solid. MS (ESI): mass calcd. for C₂₄H₂₅F₃N403, 474.2. m/z found, 475.1 [M+H]⁺. ¹H NMR (400 MHz, DMSO-d6) δ 11.10 (s, 1H), 8.54 (d, J=7.2 Hz, 1H), 8.39 (s, 1H), 7.77 (s, 1H), 7.35 (s, 1H), 7.00 (dd, J=1.6, 7.2 Hz, 1H), 6.85 (s, 1H), 5.76 (s, 1H), 4.82 (s, 1H), 2.46 (s, 3H), 1.99-1.87 (m, 1H), 1.87-1.70 (m, 4H), 1.59-1.49 (m, 2H), 1.49-1.37 (m, 2H), 0.93-0.72 (m, 4H). ¹⁹F NMR (376 MHz, DMSO-d6) δ −83.51 (s, 3H).

### Example 192: N-(5-(5-(((1r,1r)-4-hydroxy-4-phenylcyclohexyl)oxy)-2-methylpyridin-4-yl)pyrazolo[1,5-a]pyridin-2-yl)cyclopropanecarboxamide

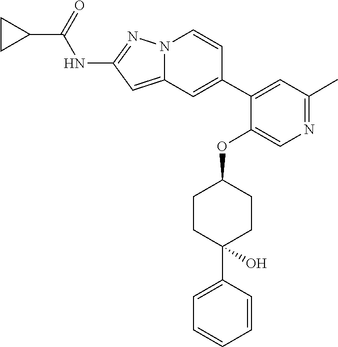 [SMILES: Cc1cc(-c2ccn3nc(NC(=O)C4CC4)cc3c2)c(O[C@H]2CC[C@@](O)(c3ccccc3)CC2)cn1]

Step A: 8-Phenyl-1,4-dioxaspiro[4.5]decan-8-ol. To a solution of 1,4-dioxaspiro[4.5]decan-8-one (1.00 g, 6.40 mmol) in THF (20 mL) was added PhMgBr (3.201 g, 9.604 mmol) at 0° C. The reaction mixture was stirred at 20° C. for 16 hours. The reaction mixture was poured into saturated ammonium chloride (40 mL) and extracted with ethyl acetate (15 mL×3). The combined organic extracts were washed with brine (40 mL), dried over anhydrous sodium sulfate, filtered, and concentrated to dryness under reduced pressure to give crude product, which was purified by FCC (eluent: petroleum ether:ethyl acetate=5:1 to 2:1) to afford the title compound (480 mg, 32%) as a light yellow solid.

Step B: 4-Hydroxy-4-phenylcyclohexan-1-one. To a solution of 8-phenyl-1,4-dioxaspiro[4.5]decan-8-ol (300 mg, 1.28 mmol) in THF (3 mL) was added HCl (3 mL, 10% HCl in water). The reaction was stirred at 25° C. for 16 hr. The reaction mixture was poured into water (10 mL) and extracted with ethyl acetate (5 mL×3). The combined organic extracts were washed with brine (10 mL), dried over anhydrous sodium sulfate, filtered, and concentrated to dryness under reduced pressure to give the title compound (240 mg, 98%) as a white solid.

Step C: 1-Phenylcyclohexane-1,4-diol. To a solution of 4-hydroxy-4-phenylcyclohexanone (240 mg, 1.26 mmol) in MeOH (4 mL) was added NaBH₄ (95.46 mg, 2.523 mmol) at 0° C. The reaction mixture was stirred at 20° C. for 3 hrs. The reaction mixture was poured into water (30 mL) and extracted with ethyl acetate (10 mL×3). The combined organic extracts were washed with brine (30 mL), dried over anhydrous sodium sulfate, filtered, and concentrated to dryness under reduced pressure to give crude product, which was purified by FCC (eluent: petroleum ether:ethyl acetate=1:1 to 1:2) to afford the title compound (160 mg, 66%) as a yellow solid.

Step D: 4-Hydroxy-4-phenylcyclohexyl 4-methylbenzenesulfonate. To a solution of 1-phenylcyclohexane-1,4-diol (100 mg, 0.520 mmol) in DCM (2 mL) was added TEA (105.3 mg. 1.040 mmol), TsCl (99.2 mg, 0.520 mmol) and DMAP (3.2 mg, 0.026 mmol). The reaction mixture was stirred at 30° C. for 2 hr. The reaction mixture was poured into water (20 mL) and extracted with DCM (10 mL×3). The combined organic extracts were washed with brine (30 mL), dried over anhydrous sodium sulfate, filtered, and concentrated to dryness under reduced pressure to give crude product, which was purified by FCC (eluent: petroleum ether: ethyl acetate=20:1 to 15:1) to afford the title compound (31 mg, 17%) as a light yellow oil.

Step E: N-(5-(5-(((1r,1r)-4-hydroxy-4-phenylcyclohexyl)oxy)-2-methylpyridin-4-yl)pyrazolo[1,5-a]pyridin-2-yl)cyclopropanecarboxamide. To a solution of N-(5-(5-hydroxy-2-methylpyridin-4-yl)pyrazolo[1,5-a]pyridin-2-yl)cyclopropanecarboxamide (Intermediate 7, 180 mg, 0.584 mmol) and 4-hydroxy-4-phenylcyclohexyl 4-methylbenzenesulfonate (202 mg, 0.584 mmol) in DMF (8 mL) was added cesium carbonate (380 mg, 1.17 mmol). The reaction mixture was stirred at 70° C. for 16 hr. The crude product was purified by preparative HPLC using a Boston Green ODS 150 mm×30 mm×5 μm column (eluent: 35% to 65% (v/v) CH₃CN and H₂O with 0.04% NH₃+10 mM NH₄HCO₃). The product was further purified by SFC YMC CHIRAL Amylose-C 250 mm×30 mm, 10 μm (eluent: 55% to 55% (v/v) supercritical CO₂ in EtOH and H₂O with 0.1% NH₃). The pure fractions were collected and the volatiles removed under reduced pressure. The product was suspended in water (10 mL), the mixture frozen using dry ice/acetone, and then lyophilized to dryness to afford compound (37.7 mg, 13%) as white solid. MS (ESI): mass calcd. for C₂₉H₃₀N₄O₃, 482.2. m/z found, 483.1 [M+H]⁺. ¹H NMR (400 MHz, DMSO-d6) δ 11.21 (s, 1H), 8.65 (d, J=7.2 Hz, 1H), 8.45 (s, 1H), 7.88 (s, 1H), 7.36 (s, 1H), 7.12-7.00 (m, 2H), 6.99-6.87 (m, 3H), 6.86-6.72 (m, 2H), 4.95 (s, 1H), 4.80 (s, 1H), 2.46 (s, 3H), 2.02-1.87 (m, 3H), 1.83-1.70 (m, 2H), 1.69-1.56 (m, 2H), 1.27-1.17 (m, 2H), 0.95-0.75 (m, 4H).

### Example 193: (*R)—N-(5-(5-(2-cyclopropyl-2-hydroxypropoxy)-2-methylpyridin-4-yl)pyrazolo[1,5-a]pyridin-2-yl)cyclopropanecarboxamide

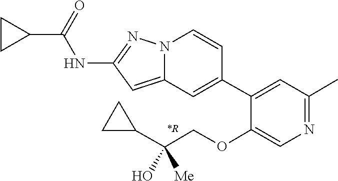 [SMILES: Cc1cc(-c2ccn3nc(NC(=O)C4CC4)cc3c2)c(OC[C@](C)(O)C2CC2)cn1]

Step A: 2-cyclopropyl-2-methyloxirane. A suspension of trimethylsulfonium iodide (6.31 g, 30.9 mmol), THF (20 mL), and t-BuOK (47.6 mL, 47.6 mmol) was stirred at rt for 30 mins. A solution of 1-cyclopropylethanone (2 g, 23.8 mmol) in THF (5 mL) was added dropwise at rt and the mixture was stirred at rt overnight. The mixture was quenched with saturated ammonium chloride (30 mL) and extracted with ethyl acetate (200 mL×2). The combined organics were dried over sodium sulfate, filtered, and concentrated to give the title compound (1.5 g, 64%) as a light yellow oil.

Step B: (rac-)-N-(5-(5-(2-cyclopropyl-2-hydroxypropoxy)-2-methylpyridin-4-yl)pyrazolo[1,5-a]pyridin-2-yl)cyclopropanecarboxamide. A mixture consisting of N-(5-(5-hydroxy-2-methylpyridin-4-yl)pyrazolo[1,5-a]pyridin-2-yl)cyclopropanecarboxamide (Intermediate 7, 400 mg, 1.30 mmol), 2-cyclopropyl-2-methyloxirane (509 mg, 5.19 mmol), cesium carbonate (1.06 g, 3.24 mmol), and DMF (10 mL) was stirred at 80° C. for 16 hr. The reaction mixture was concentrated under reduced pressure to give the crude product, which was purified by FCC (eluent: CH₂Cl₂:MeOH=1:0 to 10:1) to give crude product. The crude product was further purified by preparative HPLC with Xtimate C18 150×50 mm×10 um, column (eluent: 35% to 65% (v/v) CH₃CN and H₂O with 0.04% NH₃-H₂O+10 mM NH₄HCO₃) and lyophilized to afford the title compound (60 mg, 11%) as a white solid.

Step C: (*R)—N-(5-(5-(2-cyclopropyl-2-hydroxypropoxy)-2-methylpyridin-4-yl)pyrazolo[1,5-a]pyridin-2-yl)cyclopropanecarboxamide. (rac-)-N-(5-(5-(2-cyclopropyl-2-hydroxypropoxy)-2-methylpyridin-4-yl)pyrazolo[1,5-a]pyridin-2-yl)cyclopropanecarboxamide (60 mg, 0.148 mmol) was separated by SFC with DAICEL CHIRALPAK AD (250 mm×30 mm, 10 um), column (eluent: 50% to 50% (v/v) EtOH with 0.1% NH₃·H₂O in supercritical CO₂). The product was suspended in water (10 mL), the mixture frozen using dry ice/EtOH, and then lyophilized to dryness to afford the title compound (21.4 mg, 35%) as an off-white solid. MS (ESI): mass calcd. for C₂₃H₂₆N₄O₃, 406.2. m/z found, 407.2 [M+H]⁺. ¹H NMR (400 MHz, DMSO-d6) δ 11.11 (s, 1H), 8.57 (d, J=7.2 Hz, 1H), 8.32 (s, 1H), 7.93 (d, J=0.8 Hz, 1H), 7.37 (s, 1H), 7.11 (dd, J=1.6, 6.8 Hz, 1H), 6.88 (s, 1H), 4.37 (s, 1H), 3.97-3.90 (m, 2H), 2.46 (s, 3H), 1.98-1.89 (m, 1H), 1.14 (s, 3H), 0.94-0.87 (m, 1H), 0.86-0.79 (m, 4H), 0.42-0.35 (m, 1H), 0.34-0.26 (m, 1H), 0.26-0.15 (m, 2H).

### Example 194: trans-N-(5-(5-(((1*R,3*R)-3-hydroxycyclohexyl)oxy)-2-methylpyridin-4-yl)pyrazolo[1,5-a]pyridin-2-yl)cyclopropanecarboxamide

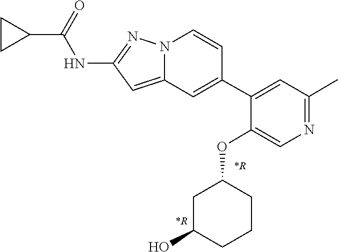 [SMILES: Cc1cc(-c2ccn3nc(NC(=O)C4CC4)cc3c2)c(O[C@@H]2CCC[C@@H](O)C2)cn1]

Step A: (1R,3S)-3-hydroxycyclohexyl 4-methylbenzenesulfonate. To a 0° C. (ice/water) mixture consisting of (1R,3S)-cyclohexane-1,3-diol (500 mg, 4.30 mmol), triethylamine (871 mg, 8.61 mmol), and DCM (4 ml) was added a solution 4-methylbenzene-1-sulfonyl chloride (820 mg, 4.30 mmol) in DCM (4 ml). The reaction was stirred at 30° C. for 16 h. Purification by FCC (petroleum ether:ethyl acetate=1:0 to 1:1) affords the crude product (270 mg, 23.2% yield) as a yellow oil.

Step B: trans-N-(5-(5-((3-hydroxycyclohexyl)oxy)-2-methylpyridin-4-yl)pyrazolo[1,5-a]pyridin-2-yl)cyclopropanecarboxamide. To a mixture consisting of N-(5-(5-hydroxy-2-methylpyridin-4-yl)pyrazolo[1,5-a]pyridin-2-yl)cyclopropanecarboxamide (Intermediate 7, 270 mg, 0.876 mmol), (1R,3S)-3-hydroxycyclohexyl 4-methylbenzenesulfonate (237 mg, 0.876 mmol) and DMF (11 mL) was added cesium carbonate (855.9 mg, 2.627 mmol). The reaction mixture was stirred at 75° C. for 16 hrs. The mixture was purified by preparative HPLC using a Boston Green ODS 150×30 5 u column (eluent: 25% to 55% (v/v) CH₃CN and H₂O with 0.05% NH₃) and lyophilized to dryness to afford the title compound (96 mg, 26.97%) as a yellow solid.

Step C: trans-N-(5-(5-(((1*R,3*R)-3-hydroxycyclohexyl)oxy)-2-methylpyridin-4-yl)pyrazolo[1,5-a]pyridin-2-yl)cyclopropanecarboxamide. The intermediate trans-N-(5-(5-((3-hydroxycyclohexyl)oxy)-2-methylpyridin-4-yl)pyrazolo[1,5-a]pyridin-2-yl)cyclopropanecarboxamide (96.0 mg, 0.236 mmol) was further purified by SFC over YMC CHIRAL Amylose-C (250 mm×30 mm, 10 um), (eluent: 55% to 55% (v/v) supercritical CO₂ in EtOH and H₂O with 0.1% NH₃). The pure fractions were collected and the volatiles removed under reduced pressure. The product was suspended in water (10 mL), the mixture frozen using dry ice/ethanol, and then lyophilized to dryness to afford the title compound (19.6 mg, 20%) as a white solid. MS (ESI): mass calcd. for C₂₃H₂₆N₄O₃, 406.2. m/z found, 407.2 [M+H]⁺. ¹H NMR (400 MHz, DMSO-d6) δ 11.12 (s, 1H), 8.58 (d, J=7.2 Hz, 1H), 8.32 (s, 1H), 7.81 (d, J=0.8 Hz, 1H), 7.35 (s, 1H), 7.04 (dd, J=1.6, 7.2 Hz, 1H), 6.89 (s, 1H), 4.81-4.73 (m, 1H), 4.54 (d, J=4.4 Hz, 1H), 3.75-3.64 (m, 1H), 2.46 (s, 3H), 1.99-1.81 (m, 2H), 1.69-1.47 (m, 5H), 1.47-1.35 (m, 1H), 1.31-1.17 (m, 1H), 0.91-0.75 (m, 4H).

### Example 195: trans-N-(5-(5-(((1*S,3*S)-3-hydroxycyclohexyl)oxy)-2-methylpyridin-4-yl)pyrazolo[1,5-a]pyridin-2-yl)cyclopropanecarboxamide

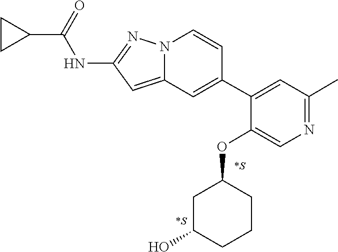 [SMILES: Cc1cc(-c2ccn3nc(NC(=O)C4CC4)cc3c2)c(O[C@H]2CCC[C@H](O)C2)cn1]

The title compound (22.7 mg, 23%, white solid) was obtained in a manner similar to Example 194, and is the other separated fraction in Step C. MS (ESI): mass calcd. for C₂₃H₂₆N₄O₃, 406.2. m/z found, 407.2 [M+H]⁺. ¹H NMR (400 MHz, DMSO-d6) δ 11.12 (s, 1H), 8.58 (d, J=7.6 Hz, 1H), 8.32 (s, 1H), 7.81 (d, J=0.8 Hz, 1H), 7.35 (s, 1H), 7.04 (dd, J=1.6, 7.2 Hz, 1H), 6.89 (s, 1H), 4.81-4.73 (m, 1H), 4.54 (d, J=4.0 Hz, 1H), 3.74-3.65 (m, 1H), 2.46 (s, 3H), 1.98-1.83 (m, 2H), 1.67-1.51 (m, 5H), 1.47-1.35 (m, 1H), 1.27-1.21 (m, 1H), 0.87-0.78 (m, 4H).

### Example 196: N-(5-(5-(4-hydroxy-4-isopropylcyclohexyl)oxy)-2-methylpyridin-4-yl)pyrazolo[1,5-a]pyridin-2-yl)cyclopropanecarboxamide

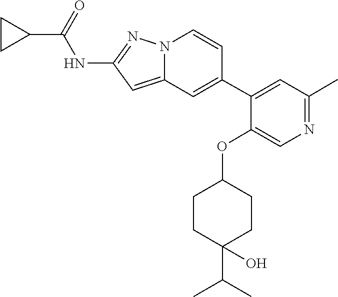 [SMILES: Cc1cc(-c2ccn3nc(NC(=O)C4CC4)cc3c2)c(OC2CCC(O)(C(C)C)CC2)cn1]

Step A: 4-(Benzyloxy)-1-isopropylcyclohexan-1-ol. To a solution of 4-(benzyloxy)cyclohexanone (5.00 g, 24.5 mmol) in THF (250 mL) was added i-PrMgCl·LiCl (20.71 mL, 26.93 mmol) at 0° C. The reaction mixture was stirred at 20° C. for 16 hours. The reaction mixture was poured into saturated ammonium chloride (200 mL) and extracted with ethyl acetate (150 mL×3). The combined organic extracts were washed with brine (400 mL), dried over anhydrous sodium sulfate, filtered, and concentrated to dryness under reduced pressure to give crude product, which was purified by Prep-HPLC (basic) to afford the title compound (980 mg, 8.0% yield) as a yellow oil.

Step B: 1-Isopropylcyclohexane-1,4-diol. To a solution of 4-(benzyloxy)-1-isopropylcyclohexanol (980 mg, 3.95 mmol) in MeOH (20 mL) was added Pd/C (100 mg, 10% in H₂O). The resultant mixture was stirred under H₂ gas (40 psi) at 40° C. for 16 hours. The mixture was filtered and concentrated to afford the title compound (650 mg, crude product) as a yellow oil.

Step C: 4-hydroxy-4-isopropylcyclohexyl 4-methylbenzenesulfonate. To a solution of 1-isopropylcyclohexane-1,4-diol (650 mg, 3.38 mmol) in DCM (20 mL) was added TEA (684.2 mg. 6.762 mmol), TsCl (709.0 mg, 3.719 mmol) and DMAP (20.7 mg, 0.169 mmol). The reaction mixture was stirred at 30° C. for 2 hr. The reaction mixture was poured into water (20 mL) and extracted with DCM (10 mL×3). The combined organic extracts were washed with brine (30 mL), dried over anhydrous sodium sulfate, filtered, and concentrated to dryness under reduced pressure to give crude product, which was purified by FCC (eluent: petroleum ether:ethyl acetate=10:1 to 1:1) to afford the title compound (110 mg, 10%) as a yellow oil.

Step D: N-(5-(5-((4-hydroxy-4-isopropylcyclohexyl)oxy)-2-methylpyridin-4-yl)pyrazolo[1,5-a]pyridin-2-yl)cyclopropanecarboxamide. To a solution of N-(5-(5-hydroxy-2-methylpyridin-4-yl)pyrazolo[1,5-a]pyridin-2-yl)cyclopropanecarboxamide (Intermediate 7, 70.0 mg, 0.227 mmol) and 4-hydroxy-4-isopropylcyclohexyl 4-methylbenzenesulfonate (70.9 mg, 0.227 mmol) in DMF (3 mL) was added cesium carbonate (148 mg, 0.454 mmol). The reaction mixture was stirred at 70° C. for 16 hr. The crude product was purified by preparative HPLC using a Boston Green ODS 150 mm×30 mm×5 μm column (eluent: 36% to 66% (v/v) CH₃CN and H₂O with 0.04% NH₃+10 mM NH₄HCO₃) and then lyophilized to dryness to afford compound (23.8 mg, 23%) as a white solid. MS (ESI): mass calcd. for C₂₆H₃₂N₄O₃, 448.3. m/z found, 449.3 [M+H]⁺. ¹H NMR (400 MHz, DMSO-d6) δ 11.09 (s, 1H), 8.55 (d, J=7.2 Hz, 1H), 8.36 (s, 1H), 7.78 (d, J=0.8 Hz, 1H), 7.33 (s, 1H), 7.01 (dd, J=2.0, 7.2 Hz, 1H), 6.86 (s, 1H), 4.80-4.74 (m, 1H), 3.76 (s, 1H), 2.45 (s, 3H), 1.97-1.89 (m, 1H), 1.85-1.75 (m, 2H), 1.71-1.62 (m, 2H), 1.39-1.23 (m, 3H), 1.14-1.05 (m, 2H), 0.86-0.77 (m, 4H), 0.54 (d, J=6.8 Hz, 6H).

### Example 197: (*S)—N-(5-(5-(2-cyclohexyl-2-hydroxyethoxy)-2-methylpyridin-4-yl)pyrazolo[1,5-a]pyridin-2-yl)cyclopropanecarboxamide

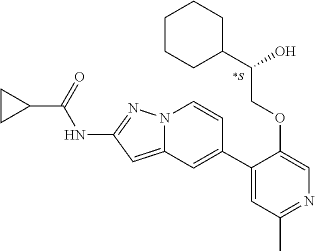 [SMILES: Cc1cc(-c2ccn3nc(NC(=O)C4CC4)cc3c2)c(OC[C@@H](O)C2CCCCC2)cn1]

Step A: Cyclohexanecarbonyl chloride. To a solution of cyclohexanecarboxylic acid (1.00 g, 7.80 mmol) in DCM (10 mL) was added oxalyl chloride (1.98 g, 15.6 mmol) and DMF (0.1 mL). The reaction mixture was stirred at 60° C. for 2 hr. The mixture was concentrated to afford the title compound (1.2 g, crude product) as yellow solid.

Step B: 2-Chloro-1-cyclohexylethan-1-one. To a solution of (trimethylsilyl)diazomethane (3.75 mL, 7.50 mmol) and TEA (759.2 mg, 7.503 mmol) in THF (15 mL) was added cyclohexanecarbonyl chloride (500 mg, 3.41 mmol) at −25° C. The reaction mixture was stirred at rt for 16 hr. To the reaction mixture was added dropwisely 1M HCl (10 mL) in THF (5 mL) at 0° C. The mixture was stirred at rt for 5 hr. The mixture was concentrated, poured into water (40 mL), and extracted with ethyl acetate (10 mL×3). The combined organic extracts were washed with brine (40 mL), dried over anhydrous sodium sulfate, filtered, and concentrated to dryness under reduced pressure to afford the title compound (370 mg, 68%) as a yellow oil.

Step C: N-(5-(5-(2-cyclohexyl-2-oxoethoxy)-2-methylpyridin-4-yl)pyrazolo[1,5-a]pyridin-2-yl)cyclopropanecarboxamide. To a solution of N-(5-(5-hydroxy-2-methylpyridin-4-yl)pyrazolo[1,5-a]pyridin-2-yl)cyclopropanecarboxamide (Intermediate 7, 255 mg, 0.830 mmol) and 2-chloro-1-cyclohexylethanone (200 mg, 1.25 mmol) in DMF (1 mL) was added cesium carbonate (540.9 mg, 1.660 mmol). The reaction mixture was stirred at 70° C. for 16 hr. The mixture was concentrated and purified by preparative HPLC using a Boston Green ODS 150 mm×30 mm×5 μm column (eluent: 41% to 71% (v/v) CH₃CN and H₂O with 0.04% NH₃+10 mM NH₄HCO₃) and lyophilized to dryness to afford compound (51 mg, 14%) as yellow solid.

Step D: (*S)—N-(5-(5-(2-cyclohexyl-2-hydroxyethoxy)-2-methylpyridin-4-yl)pyrazolo[1,5-a]pyridin-2-yl)cyclopropanecarboxamide. To a solution of N-(5-(5-(2-cyclohexyl-2-oxoethoxy)-2-methylpyridin-4-yl)pyrazolo[1,5-a]pyridin-2-yl)cyclopropanecarboxamide (40 mg, 0.092 mmol) in MeOH (2 mL) was added NaBH₄ (7.00 mg, 0.185 mmol) at 0° C. The reaction mixture was stirred at 20° C. for 2 hr. The reaction mixture was concentrated and purified by preparative SFC (eluent: 55% to 55% (v/v) 0.1% NH₃—H₂O in EtOH, DAICEL CHIRALPAK AD (250 mm*30 mm, 10 um)) and concentrated. The fractions were suspended in water (10 mL), the mixture frozen using dry ice/EtOH, and then lyophilized to dryness to afford the title compound (16.4 mg, 40.2%) as white solid. MS (ESI): mass calcd. for C₂₅H₃₀N₄O₃, 434.2. m/z found, 435.3 [M+H]⁺. ¹H NMR (400 MHz, DMSO-d6) δ 11.09 (s, 1H), 8.55 (d, J=7.2 Hz, 1H), 8.33 (s, 1H), 7.93-7.86 (m, 1H), 7.36 (s, 1H), 7.10 (dd, J=2.0, 7.2 Hz, 1H), 6.88 (s, 1H), 4.79 (d, J=5.6 Hz, 1H), 4.15-4.07 (m, 1H), 4.06-3.99 (m, 1H), 3.55-3.48 (m, 1H), 2.46 (s, 3H), 1.98-1.88 (m, 1H), 1.80-1.73. (m, 1H), 1.71-1.62 (m, 2H), 1.61-1.51 (m, 2H), 1.48-1.38 (m, 1H), 1.17-0.99 (m, 5H), 0.89-0.77 (m, 4H).

### Example 198: trans-4-[[4-[2-[(2,6-dimethylpyrimidin-4-yl)amino]pyrazolo[1,5-a]pyridin-5-yl]-6-methyl-3-pyridyl]oxy]cyclohexanol

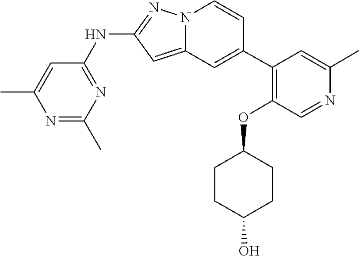

To a solution of trans-1,4-cyclohexanediol (100 mg, 0.861 mmol) in DMA (1.44 mL) was added NaH (68 mg, 60% dispersion in mineral oil). The mixture was allowed to stir at 60° C. for 5 min. To this was added, Intermediate 15 (100 mg, 0.287 mmol), and the reaction was stirred at 90° C. for 48 hr. The reaction was worked-up in water and 10% IPA/chloroform. The organic extracts were dried with sodium sulfate and purified via prep HPLC (Basic ISCO, Gemini 5 uM NX-C18 110 angstrom LC column 150×21.2 mm; 10-100% AcN/20 mM aq NH₃ over 15 min; 40 mL/min) and lyophilized to yield the title compound as a white solid (25 mg, 20%). MS (ESI): mass calcd. for C₂₅H₂₈N₆O₂, 444.2. m/z found, 445.3 [M+H]⁺. ¹H NMR (500 MHz, DMSO-d6) δ 10.12 (s, 1H), 8.53-8.48 (m, 1H), 8.28 (s, 1H), 7.74-7.66 (m, 1H), 7.27 (s, 1H), 6.93 (dd, J=7.2, 2.0 Hz, 1H), 6.78 (s, 1H), 5.68 (s, 1H), 4.45 (d, J=4.0 Hz, 1H), 4.39-4.27 (m, 1H), 3.48-3.38 (m, 1H), 2.39 (d, J=2.1 Hz, 6H), 2.23 (s, 3H), 1.95-1.85 (m, 2H), 1.70-1.62 (m, 2H), 1.42-1.28 (m, 2H), 1.26-1.17 (m, 2H).

### Example 199: N-[5-[5-(4-hydroxyiminocyclohexoxy)-2-methyl-4-pyridyl]pyrazolo[1,5-a]pyridin-2-yl]cyclopropanecarboxamide

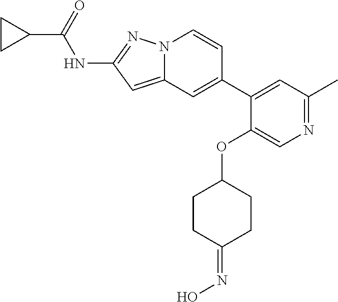 [SMILES: Cc1cc(-c2ccn3nc(NC(=O)C4CC4)cc3c2)c(OC2CCC(=NO)CC2)cn1]

Step A: N-(5-(5-((1,4-dioxaspiro[4.5]decan-8-yl)oxy)-2-methylpyridin-4-yl)pyrazolo[1,5-a]pyridin-2-yl)cyclopropanecarboxamide. NaH (0.644 g, 16.1 mmol, 60% in mineral oil) was added to a solution consisting of 1,4-dioxaspiro[4.5]decan-8-ol (2.55 g, 16.1 mmol) in 12.5 mL DMA. The resulting mixture was stirred at 80° C. for 30 min. To this was added dropwisely a solution of N-(5-(5-fluoro-2-methylpyridin-4-yl)pyrazolo[1,5-a]pyridin-2-yl)cyclopropanecarboxamide (Intermediate 6, 1.00 g, 3.22 mmol) in 7.5 mL DMA at 80° C. The mixture was allowed to stir at 80° C. for 1 hr. The combined reaction mixture was quenched with water (20 mL) and diluted with ethyl acetate (100 mL). The organic layer was separated, washed with brine (30 mL×3), dried over anhydrous sodium sulfate, filtered and concentrated to dryness under reduced pressure to afford the crude product which was purified by FCC (petroleum ether:ethyl acetate=2:3 to 0:1) to afford the title compound as white solid.

Step B: N-(5-(2-methyl-5-((4-oxocyclohexyl)oxy)pyridin-4-yl)pyrazolo[1,5-a]pyridin-2-yl)cyclopropanecarboxamide. To a mixture consisting of N-(5-(5-(1,4-dioxaspiro[4.5]decan-8-yloxy)-2-methylpyridin-4-yl)pyrazolo[1,5-a]pyridin-2-yl)cyclopropanecarboxamide (1.20 g, 2.68 mmol) and DCM (60 mL) was added FeCl₃·6H₂O (4.34 g, 16.1 mmol) at 28° C. The reaction mixture was stirred at rt for 37 hrs. The reaction mixture was diluted with 40 mL DCM, adjusted to pH 8 with aqueous NaHCO₃, and filtered through a pad of Celite. The pad was rinsed with DCM (100 mL). The organic layer was separated and the aqueous layer was extracted with DCM (50 mL×2). The combined organics were dried over sodium sulfate, filtered and concentrated to dryness under reduced pressure to give the crude product. The product was suspended in MeCN (10 mL) and water (50 mL), and the mixture frozen using dry ice/ethanol, and then lyophilized to afford the title compound (1.05 g, 88%) as a light yellow solid.

Step C: N-[5-[5-(4-hydroxyiminocyclohexoxy)-2-methyl-4-pyridyl]pyrazolo[1,5-a]pyridin-2-yl]cyclopropanecarboxamide. A mixture consisting of N-(5-(2-methyl-5-((4-oxocyclohexyl)oxy)pyridin-4-yl)pyrazolo[1,5-a]pyridin-2-yl)cyclopropanecarboxamide (200 mg, 0.494 mmol), hydroxylamine (49.0 mg, 0.742 mmol), water (1.6 ml) and EtOH (4 mL) was stirred at 80° C. for 16 h. The mixture was concentrated under reduced pressure and purified by preparative HPLC using a Phenomenex Gemini-NX C18 75×30 mm×3 um column (eluent: 25% to 55% (v/v) CH₃CN and H₂O with 0.04% NH₃) and lyophilized to dryness to afford the title compound (149.9 mg, 72.3%) as a white solid. MS (ESI): mass calcd. for C₂₃H₂₅N₅O₃, 419.2. m/z found, 420.2 [M+H]⁺. ¹H NMR (400 MHz, CDCl₃) δ 9.17 (s, 1H), 9.04 (s, 1H), 8.31 (s, 1H), 8.27 (d, J=6.8 Hz, 1H), 7.63 (s, 1H), 7.20 (s, 1H), 7.02 (s, 1H), 6.97 (dd, J=1.6, 7.2 Hz, 1H), 4.63-4.56 (m, 1H), 2.70-2.60 (m, 1H), 2.56 (s, 3H), 2.50-2.40 (m, 1H), 2.37-2.28 (m, 1H), 2.22-2.13 (m, 1H), 2.01-1.80 (m, 4H), 1.55-1.60 (m, 1H), 1.18-1.10 (m, 2H), 0.93-0.85 (m, 2H).

### Example 200: 1-[[4-[2-[(2,6-dimethylpyrimidin-4-yl)amino]pyrazolo[1,5-a]pyridin-5-yl]-6-methyl-3-pyridyl]oxy]-2-methyl-propan-2-ol

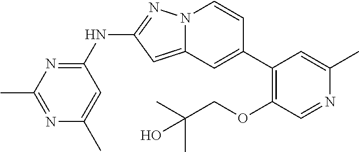

To a microwave vial was added 1-((4-(2-aminopyrazolo[1,5-a]pyridin-5-yl)-6-methylpyridin-3-yl)oxy)-2-methylpropan-2-ol (Intermediate 20, 100 mg, 0.32 mmol), 4-chloro-2,6-dimethylpyrimidine (50 mg, 0.35 mmol), cesium carbonate (313 mg, 0.96 mmol), and t-Bu-BRETTPhos Pd G3 (13.7 mg, 0.016 mmol). The vial was sealed and injected with 1,4-dioxane (0.64 mL) under vacuum. An argon balloon was inserted, then the reaction was heated at 90° C. for 4 hr. The reaction mixture was diluted with ethyl acetate, then filtered through Celite. The filtrate was concentrated under vacuum, then purified via FCC (10% MeOH (with 2M NH₃) in DCM) to yield the title compound (68 mg, 50%) as a yellow solid. MS (ESI): mass calcd. for C₂₃H₂₆N₆O₂, 418.2. m/z found, 419.2 [M+H]⁺. ¹H NMR (400 MHz, CD₃OD) δ 8.48-8.41 (m, 1H), 8.24 (s, 1H), 7.85-7.78 (m, 1H), 7.39 (s, 1H), 7.08 (dd, J=7.2, 2.0 Hz, 1H), 7.06-7.03 (m, 1H), 6.87 (s, 1H), 3.93 (s, 2H), 2.53 (d, J=2.3 Hz, 6H), 2.38 (s, 3H), 1.26 (s, 6H).

### Example 201: 1-[[4-[2-[(5,6-dimethylpyridazin-3-yl)amino]pyrazolo[1,5-a]pyridin-5-yl]-6-methyl-3-pyridyl]oxy]-2-methyl-propan-2-ol

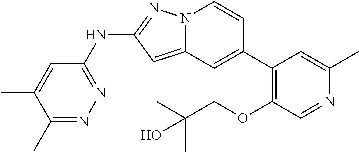

The title compound was prepared (77 mg, 58%, yellow solid) in a manner similar to Example 200, using 6-chloro-3,4-dimethylpyridazine instead of 4-chloro-2,6-dimethylpyrimidine. MS (ESI): mass calcd. for C₂₃H₂₆N₆O₂, 418.2. m/z found, 419.3 [M+H]⁺. ¹H NMR (500 MHz, DMSO-d6) δ 9.91 (s, 1H), 8.59-8.53 (m, 1H), 8.32 (s, 1H), 7.93-7.86 (m, 1H), 7.40-7.35 (m, 2H), 7.04 (dd, J=7.2, 2.0 Hz, 1H), 6.91-6.86 (m, 1H), 4.66 (s, 1H), 3.88 (s, 2H), 2.47 (d, J=3.5 Hz, 6H), 2.25 (s, 3H), 1.17 (s, 6H).

### Example 202: 1-[[4-[2-[(2-ethyl-6-methyl-pyrimidin-4-yl)amino]pyrazolo[1,5-a]pyridin-5-yl]-6-methyl-3-pyridyl]oxy]-2-methyl-propan-2-ol

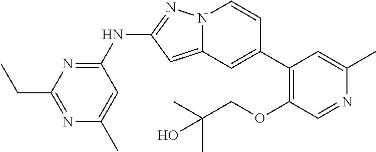 [SMILES: CCc1nc(C)cc(Nc2cc3cc(-c4cc(C)ncc4OCC(C)(C)O)ccn3n2)n1]

The title compound was prepared (53 mg, 38%, white solid) in a manner similar to Example 200, using 4-chloro-2-ethyl-6-methylpyrimidine instead of 4-chloro-2,6-dimethylpyrimidine. MS (ESI): mass calcd. for C₂₄H₂₈N₆O₂, 432.2. m/z found, 433.3 [M+H]⁺. ¹H NMR (500 MHz, DMSO-d6) δ 10.13 (s, 1H), 8.54-8.49 (m, 1H), 8.24 (s, 1H), 7.84-7.79 (m, 1H), 7.31 (s, 1H), 7.01 (dd, J=7.2, 2.0 Hz, 1H), 6.96-6.79 (m, 2H), 4.57 (s, 1H), 3.81 (s, 2H), 2.67 (q, J=7.6 Hz, 2H), 2.40 (s, 3H), 2.24 (s, 3H), 1.23 (t, J=7.6 Hz, 3H), 1.09 (s, 6H).

### Example 203: (*R)-3-(((4-(2-((2,6-dimethylpyrimidin-4-yl)amino)pyrazolo[1,5-a]pyridin-5-yl)-6-methylpyridin-3-yl)oxy)methyl)tetrahydrofuran-3-ol

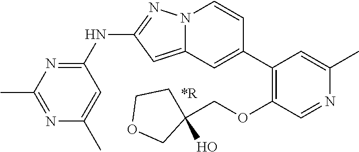

Step A: (3-hydroxytetrahydrofuran-3-yl)methyl 4-methylbenzenesulfonate. To a mixture consisting of 3-(hydroxymethyl)tetrahydrofuran-3-ol (200 mg, 1.69 mmol), TEA (308 mg, 3.05 mmol), DMAP (20.7 mg, 0.169 mmol) and DCM (5 mL) was added TsCl (339 mg, 1.78 mmol). The mixture was stirred at 25° C. for 1.5 h. The reaction mixture was washed with 1N HCl (10 mL×2) and saturated aqueous NaHCO₃ (10 mL×2), then concentrated under reduced pressure to afford the title compound as a yellow oil (300 mg, 65%).

Step B: (rac-)3-(((4-(2-((2,6-dimethylpyrimidin-4-yl)amino)pyrazolo[1,5-a]pyridin-5-yl)-6-methylpyridin-3-yl)oxy)methyl)tetrahydrofuran-3-ol. A mixture consisting of Intermediate 16 (223 mg, 0.643 mmol), (3-hydroxytetrahydrofuran-3-yl)methyl 4-methylbenzenesulfonate (140 mg, 0.514 mmol), cesium carbonate (419 mg, 1.29 mmol) and DMF (2 mL) was stirred for 1 h at 110° C. in the microwave. The mixture was purified by preparative HPLC with a Phenomenex Gemini-NX 150×30 mm×5 um column (eluent: 23% to 53% (v/v) CH₃CN and H₂O with 0.04% NH₃—H₂O) and lyophilized to dryness to the title compound (110 mg, 38%) as a white solid.

Step C: (*R)-3-(((4-(2-((2,6-dimethylpyrimidin-4-yl)amino)pyrazolo[1,5-a]pyridin-5-yl)-6-methylpyridin-3-yl)oxy)methyl)tetrahydrofuran-3-ol. (rac-)-3-(((4-(2-((2,6-dimethylpyrimidin-4-yl)amino)pyrazolo[1,5-a]pyridin-5-yl)-6-methylpyridin-3-yl)oxy)methyl)tetrahydrofuran-3-ol (160 mg, 0.358 mmol) was further purified by SFC-11 over DAICEL CHIRALPAK AD 250 mm×30 mm×10 μm (eluent: 45% to 45% (v/v) EtOH with 0.1% NH₃·H₂O in supercritical CO₂). The pure fractions were collected and the volatiles removed under reduced pressure. The product was suspended in water (10 mL), the mixture frozen using dry ice/EtOH, and then lyophilized to dryness to afford the title compound (67 mg, 42%). MS (ESI): mass calcd. for C₂₄H₂₆N₆O₃, 446.2. m/z found, 447.2 [M+H]⁺. ¹H NMR (400 MHz, CDCl₃) δ 8.33 (d, J=7.2 Hz, 1H), 8.28 (s, 1H), 7.67 (s, 1H), 7.57 (s, 1H), 7.17 (s, 1H), 6.99 (s, 1H), 6.87 (dd, J=2.0, 7.2 Hz, 1H), 6.58 (s, 1H), 4.25-4.19 (m, 1H), 4.18-4.13 (m, 1H), 4.10-4.01 (m, 1H), 3.97-3.90 (m, 1H), 3.86-3.76 (m, 2H), 3.17 (s, 1H), 2.58 (s, 3H), 2.57 (s, 3H), 2.43 (s, 3H), 2.09-1.99 (m, 2H).

### Example 204: (3R,5R)-5-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol

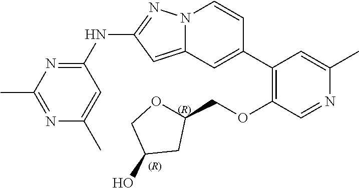

Step A: ((2R,4R)-4-hydroxytetrahydrofuran-2-yl)methyl 4-methylbenzenesulfonate. To a mixture consisting of (3R,5R)-5-(hydroxymethyl)tetrahydrofuran-3-ol (300 mg, 2.54 mmol), TEA (514 mg, 5.08 mmol), DMAP (31 mg, 0.25 mmol) and DCM (5 mL) was added TsCl (508 mg, 2.67 mmol). The mixture was stirred at 25° C. for 1.5 h. The reaction mixture was washed with 1N HCl (10 mL×2) and saturated aqueous NaHCO₃ (10 mL×2), then concentrated under reduced pressure to afford the title compound as a yellow oil (500 mg, 72%).

Step B: (3R,5R)-5-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol. A mixture consisting of Intermediate 16 (120 mg, 0.346 mmol), ((2R,4R)-4-hydroxytetrahydrofuran-2-yl)methyl 4-methylbenzenesulfonate (99 mg, 0.36 mmol), cesium carbonate (226 mg, 0.693 mmol) and DMF (2 mL) was stirred in the microwave at 110° C. The mixture was purified by preparative HPLC with a Phenomenex Gemini-NX 150×30 mm×5 um column (eluent: 23% to 53% (v/v) CH₃CN and H₂O with 0.04% NH₃—H₂O) and lyophilized to dryness. The product was impure, and was re-purified via SFC over AD 250 mm×50 mm, 10 μm (eluent: 40% to 40% (v/v) supercritical CO₂ in EtOH and H₂O with 0.1% NH₃) to yield the title compound. MS (ESI): mass calcd. for C₂₄H₂₆N₆O₃, 446.2. m/z found, 447.1 [M+H]⁺. ¹H NMR (400 MHz, DMSO-d6) δ 10.21 (s, 1H), 8.57 (d, J=7.2 Hz, 1H), 8.34 (s, 1H), 7.88 (s, 1H), 7.37 (s, 1H), 7.11-7.06 (m, 1H), 7.01 (s, 1H), 6.87 (s, 1H), 4.98 (d, J=4.0 Hz, 1H), 4.37-4.28 (m, 1H), 4.26-4.13 (m, 3H), 3.71-3.66 (m, 1H), 3.65-3.60 (m, 1H), 2.49-2.44 (m, 6H), 2.30 (s, 3H), 2.23-2.14 (m, 1H), 1.69-1.58 (m, 1H).

### Example 205: 1-[[4-[2-[[2-(methoxymethyl)-6-methyl-pyrimidin-4-yl]amino]pyrazolo[1,5-a]pyridin-5-yl]-6-methyl-3-pyridyl]oxy]-2-methyl-propan-2-ol

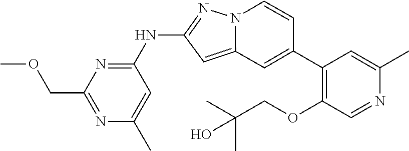

The title compound was prepared (73 mg, 43%, white solid) in a manner similar to Example 200, using 4-chloro-2-(methoxymethyl)-6-methylpyrimidine instead of 4-chloro-2,6-dimethylpyrimidine. MS (ESI): mass calcd. for C₂₄H₂₈N₆O₃, 448.2. m/z found, 449.2 [M+H]⁺. ¹H NMR (500 MHz, DMSO-d6) δ10.28 (s, 1H), 8.56-8.48 (m, 1H), 8.25 (s, 1H), 7.88-7.77 (m, 1H), 7.31 (s, 1H), 7.02 (dd, J=7.2, 2.0 Hz, 2H), 6.83 (s, 1H), 4.57 (s, 1H), 4.35 (s, 2H), 3.81 (s, 2H), 3.34 (s, 3H), 2.40 (s, 3H), 2.28 (s, 3H), 1.09 (s, 6H).

### Example 206: N-(2,6-dimethylpyrimidin-4-yl)-5-(2-methyl-5-(((2*S,3*R)-2-methyltetrahydrofuran-3-yl)methoxy)pyridin-4-yl)pyrazolo[1,5-a]pyridin-2-amine

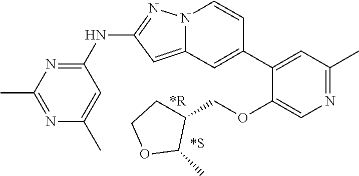

Step A: cis-(2-methyltetrahydrofuran-3-yl)methyl 4-methylbenzenesulfonate. A solution consisting of ((cis)-2-methyltetrahydrofuran-3-yl)methanol (500 mg, 4.30 mmol), DMAP (789 mg, 6.46 mmol), and dichloromethane (10 mL) was treated with TsCl (1.23 g, 6.46 mmol) at room-temperature. The resultant mixture was stirred at room-temperature for 16 hr. The mixture was washed with HCl (5 mL×2, 1 M), then with NaHCO₃ aq. (5 mL×2) then finally with water (5 mL×2). The organic layer was dried over sodium sulfate, and concentrated to dryness to afford the title compound (721 mg, 62.0%) as a colorless oil.

Step B: N-(2,6-dimethylpyrimidin-4-yl)-5-(2-methyl-5-(((cis)-2-methyltetrahydrofuran-3-yl)methoxy)pyridin-4-yl)pyrazolo[1,5-a]pyridin-2-amine. A mixture consisting of Intermediate 16 (894 mg, 2.58 mmol), ((cis)-2-methyltetrahydrofuran-3-yl)methyl 4-methylbenzenesulfonate (698 mg, 2.58 mmol), cesium carbonate (2.52 g, 7.75 mmol), and DMF (5 mL) was heated to 110° C. via microwave irradiation for 1 hour before cooling to room-temperature. The reaction mixture was added with water (20 mL) and extracted with ethyl acetate (20 mL×3). The combined organic extracts were concentrated and purified by preparative HPLC using a Boston Prime C18 150×30 mm×5 μm column (eluent: 35% to 65% (v/v) CH₃CN and H₂O with 0.04% NH₃-H₂O+10 mM NH₄HCO₃) and lyophilized to dryness to afford the title compound (334 mg, total yield: 28%) as a white solid.

Step C: N-(2,6-dimethylpyrimidin-4-yl)-5-(2-methyl-5-(((2*S,3*R)-2-methyltetrahydrofuran-3-yl)methoxy)pyridin-4-yl)pyrazolo[1,5-a]pyridin-2-amine. N-(2,6-dimethylpyrimidin-4-yl)-5-(2-methyl-5-(((cis)-2-methyltetrahydrofuran-3-yl)methoxy)pyridin-4-yl)pyrazolo[1,5-a]pyridin-2-amine (334 mg, 0.751 mmol) was purified by SFC over DAICEL CHIRALPAK AD-H 250 mm×30 mm×5 μm (eluent: 40% to 40% (v/v) IPA with 0.1% NH₃·H₂O in supercritical CO₂). The pure fractions were collected and the volatiles removed under reduced pressure. The product was suspended in water (10 mL), the mixture frozen using dry ice/EtOH, and then lyophilized to dryness to afford the title compound (114 mg, 34%). MS (ESI): mass calcd. for C₂₅H₂₈N₆O₂, 444.2. m/z found, 445.1 [M+H]⁺. ¹H NMR (400 MHz, DMSO-d6) δ 10.23 (s, 1H), 8.60 (d, J=7.2 Hz, 1H), 8.38 (s, 1H), 7.81 (s, 1H), 7.36 (s, 1H), 7.05-6.98 (m, 2H), 6.85 (s, 1H), 4.18-4.10 (m, 1H), 4.08-4.02 (m, 1H), 4.00-3.92 (m, 1H), 3.86-3.77 (m, 1H), 3.59-3.50 (m, 1H), 2.58-2.54 (m, 1H), 2.47 (s, 3H), 2.46 (s, 3H), 2.30 (s, 3H), 2.06-1.94 (m, 1H), 1.81-1.68 (m, 1H), 1.04 (d, J=6.4 Hz, 3H).

### Example 207: N-(2,6-dimethylpyrimidin-4-yl)-5-(2-methyl-5-(((2*R,3*S)-2-methyltetrahydrofuran-3-yl)methoxy)pyridin-4-yl)pyrazolo[1,5-a]pyridin-2-amine

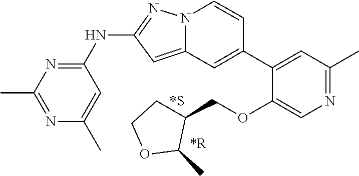

The title compound (126 mg, 38%, white solid) was obtained in a manner similar to Example 206, and is the other separated fraction in Step C. MS (ESI): mass calcd. for C₂₅H₂₈N₆O₂, 444.2. m/z found, 445.1 [M+H]⁺. ¹H NMR (400 MHz, DMSO-d6) δ 10.23 (s, 1H), 8.60 (d, J=7.2 Hz, 1H), 8.38 (s, 1H), 7.81 (d, J=0.8 Hz, 1H), 7.36 (s, 1H), 7.07-6.98 (m, 2H), 6.85 (s, 1H), 4.16-4.10 (m, 1H), 4.08-4.02 (m, 1H), 4.00-3.92 (m, 1H), 3.85-3.78 (m, 1H), 3.59-3.51 (m, 1H), 2.58-2.54 (m, 1H), 2.47 (s, 3H), 2.46 (s, 3H), 2.30 (s, 3H), 2.05-1.95 (m, 1H), 1.79-1.69 (m, 1H), 1.04 (d, J=6.4 Hz, 3H).

### Example 208: 3-[[5-[5-(2-hydroxy-2-methyl-propoxy)-2-methyl-4-pyridyl]pyrazolo[1,5-a]pyridin-2-yl]amino]-5-methyl-1H-pyridazin-6-one

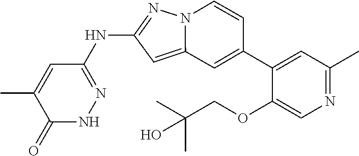

The title compound was prepared (106 mg, 26%, white solid) in a manner similar to Example 200, using 6-chloro-4-methylpyridazin-3(2h)-one instead of 4-chloro-2,6-dimethylpyrimidine. MS (ESI): mass calcd. for C₂₂H₂₄N₆O₃, 420.2. m/z found, 421.2 [M+H]⁺. ¹H NMR (500 MHz, DMSO-d6) δ 12.07 (s, 1H), 9.65 (s, 1H), 8.56-8.49 (m, 1H), 8.31 (s, 1H), 7.90-7.81 (m, 1H), 7.36 (s, 1H), 7.28-7.19 (m, 1H), 7.05 (dd, J=7.2, 2.0 Hz, 1H), 6.77-6.71 (m, 1H), 4.64 (s, 1H), 3.88 (s, 2H), 2.47 (s, 3H), 2.06 (d, J=1.3 Hz, 3H), 1.16 (s, 6H).

### Example 209: 6-[[5-[5-(2-hydroxy-2-methyl-propoxy)-2-methyl-4-pyridyl]pyrazolo[1,5-a]pyridin-2-yl]amino]-2,4-dimethyl-pyridazin-3-one

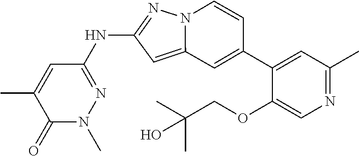

The title compound was prepared (13.5 mg, 9%, white solid) in a manner similar to Example 200, using 6-chloro-2,4-dimethylpyridazin-3(2h)-one instead of 4-chloro-2,6-dimethylpyrimidine. MS (ESI): mass calcd. for C₂₃H₂₆N₆O₃, 434.2. m/z found, 435.2 [M+H]⁺. ¹H NMR (400 MHz, DMSO-d6) δ 9.67 (s, 1H), 8.52-8.41 (m, 1H), 8.24 (s, 1H), 7.77 (dd, J=2.0, 0.9 Hz, 1H), 7.29 (s, 1H), 7.17 (d, J=1.5 Hz, 1H), 7.00 (dd, J=7.1, 2.0 Hz, 1H), 6.82-6.66 (m, 1H), 4.55 (s, 1H), 3.80 (s, 2H), 3.54 (s, 3H), 2.39 (s, 3H), 2.06-1.97 (m, 3H), 1.08 (s, 6H).

### Example 210: (*S)—N-[5-[5-[(3-cyanopyrrolidin-3-yl)methoxy]-2-methyl-4-pyridyl]pyrazolo[1,5-a]pyridin-2-yl]cyclopropanecarboxamide

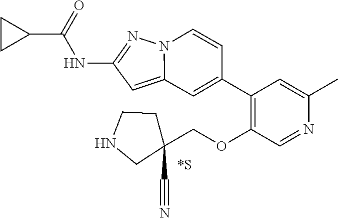 [SMILES: Cc1cc(-c2ccn3nc(NC(=O)C4CC4)cc3c2)c(OC[C@@]2(C#N)CCNC2)cn1]

Step A: (*S)—N-(5-(5-((1-benzyl-3-cyanopyrrolidin-3-yl)methoxy)-2-methylpyridin-4-yl)pyrazolo[1,5-a]pyridin-2-yl)cyclopropanecarboxamide. The title compound was prepared (7 mg, 7% yield) by the similar method of Example 1 by using 1-benzyl-3-(hydroxymethyl)pyrrolidine-3-carbonitrile instead of (R)-(1-methylpyrrolidin-3-yl)methanol and Intermediate 6 instead of N-(5-(5-chloro-2-cyanopyridin-4-yl)pyrazolo[1,5-a]pyridin-2-yl)cyclopropanecarboxamide. The resulting racemate N-(5-(5-((1-benzyl-3-cyanopyrrolidin-3-yl)methoxy)-2-methylpyridin-4-yl)pyrazolo[1,5-a]pyridin-2-yl)cyclopropanecarboxamide (320 mg, 0.632 mmol) was purified by SFC over DAICEL CHIRALCEL OJ-H 250 mm×30 mm, 5 μm (eluent: 50% to 50% (v/v) supercritical 0.1% NH₃—H₂O ETOH). The pure fractions were collected and the volatiles were removed under reduced pressure. The product was suspended in water (10 mL), the mixture frozen using dry ice/acetone, and then lyophilized to dryness to afford (*R)—N-(5-(5-((l-benzyl-3-cyanopyrrolidin-3-yl)methoxy)-2-methylpyridin-4-yl)pyrazolo[1,5-a]pyridin-2-yl)cyclopropanecarboxamide (110 mg, 27%) as a yellow solid and (*S)—N-(5-(5-((1-benzyl-3-cyanopyrrolidin-3-yl)methoxy)-2-methylpyridin-4-yl)pyrazolo[1,5-a]pyridin-2-yl)cyclopropanecarboxamide (170 mg, 52%) as a white solid.

Step B: (*S)—N-[5-[5-[(3-cyanopyrrolidin-3-yl)methoxy]-2-methyl-4-pyridyl]pyrazolo[1,5-a]pyridin-2-yl]cyclopropanecarboxamide. A mixture of 10% Pd/C (30 mg, dry) and MeOH (4 mL) was added HCOO·NH₄ (90 mg, 1.4 mmol) and (*S)—N-(5-(5-((1-benzyl-3-cyanopyrrolidin-3-yl)methoxy)-2-methylpyridin-4-yl)pyrazolo[1,5-a]pyridin-2-yl)cyclopropanecarboxamide (60 mg, 0.12 mmol), then the reaction was stirred at 55° C. for 16 h. The suspension was filtered through a pad of Celite® and the pad washed with MeOH (10 mL). The filtrate was concentrated to dryness under reduced pressure to afford the title product. The post chromatographic product was further purified by preparative HPLC using a Waters Xbridge Prep OBD C18 150×40 mm×10 μm column (eluent: 0% to 60% (v/v) water (10 mM NH₄HCO₃)-ACN) to afford pure product. The product was suspended in water (10 mL), the mixture frozen using dry ice/ethanol, and then lyophilized to dryness to afford the title compound (2.2 mg, 4%) as a white solid. MS (ESI): mass calcd. for C₂₃H₂₄N₆O₂, 416.2. m/z found, 417.2 [M+H]⁺. ¹H NMR (400 MHz, CD₃OD) δ 8.42 (d, J=7.6 Hz, 1H), 8.26 (s, 1H), 7.84 (d, J=1.2 Hz, 1H), 7.41 (s, 1H), 7.09 (dd, J=2.0, 7.6 Hz, 1H), 6.92 (s, 1H), 4.58 (s, 1H), 4.31-4.18 (m, 2H), 3.21-3.15 (m, 1H), 3.11-3.06 (m, 1H), 3.03-2.95 (m, 2H), 2.54 (s, 3H), 2.32-2.18 (m, 1H), 2.07-1.94 (m, 1H), 1.91-1.80 (m, 1H), 1.03-0.97 (m, 2H), 0.93-0.87 (m, 2H).

### Example 211: (*R)—N-[5-[5-[[(3R)-3-cyanopyrrolidin-3-yl]methoxy]-2-methyl-4-pyridyl]pyrazolo[1,5-a]pyridin-2-yl]cyclopropanecarboxamide

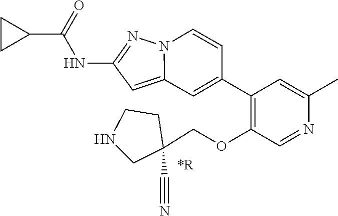 [SMILES: Cc1cc(-c2ccn3nc(NC(=O)C4CC4)cc3c2)c(OC[C@]2(C#N)CCNC2)cn1]

The title compound was prepared (2 mg, 4% yield) by the similar method of Example 210 Example by using (*R)—N-(5-(5-((1-benzyl-3-cyanopyrrolidin-3-yl)methoxy)-2-methylpyridin-4-yl)pyrazolo[1,5-a]pyridin-2-yl)cyclopropanecarboxamide instead of (*S)—N-(5-(5-((1-benzyl-3-cyanopyrrolidin-3-yl)methoxy)-2-methylpyridin-4-yl)pyrazolo[1,5-a]pyridin-2-yl)cyclopropanecarboxamide in step B. MS (ESI): mass calcd. for C₂₃H₂₄N₆O₂, 416.2. m/z found, 417.1 [M+H]⁺. ¹H NMR (400 MHz, CD₃OD) δ 8.43 (d, J=6.8 Hz, 1H), 8.26 (s, 1H), 7.85 (d, J=0.8 Hz, 1H), 7.42 (s, 1H), 7.10 (dd, J=1.6, 7.2 Hz, 1H), 6.92 (s, 1H), 4.36-4.15 (m, 2H), 3.21-3.14 (m, 1H), 3.11-3.04 (m, 1H), 3.03-2.94 (m, 2H), 2.54 (s, 3H), 2.33-2.16 (m, 1H), 2.06-1.96 (m, 1H), 1.92-1.82 (m, 1H), 1.04-0.95 (m, 2H), 0.94-0.86 (m, 2H).

### Example 212: (*S)—N-[5-[5-[(3-cyano-1-methyl-pyrrolidin-3-yl)methoxy]-2-methyl-4-pyridyl]pyrazolo[1,5-a]pyridin-2-yl]cyclopropanecarboxamide

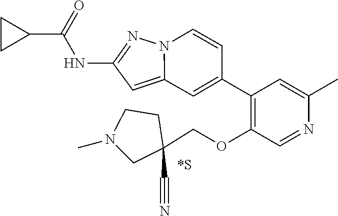 [SMILES: Cc1cc(-c2ccn3nc(NC(=O)C4CC4)cc3c2)c(OC[C@@]2(C#N)CCN(C)C2)cn1]

To a mixture of 10% Pd/C (50 mg, dry) and MeOH (2 mL) was added HCOO·NH₄ (44.8 mg, 0.711 mmol), HCl (0.118 mL, 0.118 mmol) and (*S)—N-(5-(5-((1-benzyl-3-cyanopyrrolidin-3-yl)methoxy)-2-methylpyridin-4-yl)pyrazolo[1,5-a]pyridin-2-yl)cyclopropanecarboxamide (30 mg, 0.059 mmol), then the reaction was stirred at 50° C. for 16 h. The suspension was filtered through a pad of Celite® and the pad washed with ethyl acetate (10 mL). The filtrate was concentrated to dryness under reduced pressure. The product was purified by preparative HPLC using a YMC-Triart Prep C18 250×50 mm×10 μm column (eluent: 25% to 55% (v/v) water (0.04% NH₃-H₂O+10 mM NH₄HCO₃)-ACN) to afford pure product. The product was suspended in water (10 mL), the mixture frozen using dry ice/ethanol, and then lyophilized to dryness to afford the title compound (2 mg, 7%) as a white solid. MS (ESI): mass calcd. for C₂₄H₂₆N₆O₂, 430.2. m/z found, 431.2 [M+H]⁺. ¹H NMR (400 MHz, CD₃OD) δ 8.42 (d, J=6.8 Hz, 1H), 8.26 (s, 1H), 7.84 (s, 1H), 7.42 (s, 1H), 7.16-7.02 (m, 1H), 6.92 (s, 1H), 4.39-4.15 (m, 2H), 2.97-2.84 (m, 1H), 2.77-2.67 (m, 2H), 2.63-2.57 (m, 1H), 2.54 (s, 3H), 2.31 (s, 3H), 2.30-2.24 (m, 1H), 2.14-2.03 (m, 1H), 1.91-1.82 (m, 1H), 1.02-0.96 (m, 2H), 0.94-0.88 (m, 2H).

### Example 213: (*R)—N-[5-[5-[(3-cyano-1-methyl-pyrrolidin-3-yl)methoxy]-2-methyl-4-pyridyl]pyrazolo[1,5-a]pyridin-2-yl]cyclopropanecarboxamide

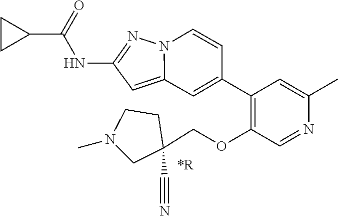 [SMILES: Cc1cc(-c2ccn3nc(NC(=O)C4CC4)cc3c2)c(OC[C@]2(C#N)CCN(C)C2)cn1]

The title compound was isolated as a secondary product from Example 211 to afford the title compound (3 mg, 6%). MS (ESI): mass calcd. for C₂₄H₂₆N₆O₂, 430.2. m/z found, 431.2 [M+H]⁺. ¹H NMR (400 MHz, CD₃OD) δ 8.42 (d, J=7.6 Hz, 1H), 8.25 (s, 1H), 7.83 (d, J=1.2 Hz, 1H), 7.41 (s, 1H), 7.09 (dd, J=2.0, 7.2 Hz, 1H), 6.92 (s, 1H), 4.33-4.17 (m, 2H), 2.93-2.85 (m, 1H), 2.76-2.67 (m, 2H), 2.63-2.56 (m, 1H), 2.54 (s, 3H), 2.31 (s, 3H), 2.30-2.24 (m, 1H), 2.13-2.04 (m, 1H), 1.93-1.82 (m, 1H), 1.05-0.95 (m, 2H), 0.94-0.83 (m, 2H).

### Example 214: (*R)—N-[5-[5-[(3-methoxypyrrolidin-3-yl)methoxy]-2-methyl-4-pyridyl]pyrazolo[1,5-a]pyridin-2-yl]cyclopropanecarboxamide

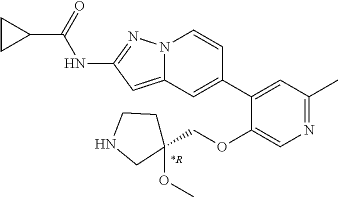 [SMILES: CO[C@]1(COc2cnc(C)cc2-c2ccn3nc(NC(=O)C4CC4)cc3c2)CCNC1]

The title compound was prepared (7 mg, 7% yield) by the similar method of Example 8 by using tert-butyl 3-(hydroxymethyl)-3-methoxypyrrolidine-1-carboxylate instead of (2S,4R)-tert-butyl 4-hydroxy-2-(trifluoromethyl)piperidine-1-carboxylate and Intermediate 6 instead of Intermediate 4. N-(5-(5-((3-methoxypyrrolidin-3-yl)methoxy)-2-methylpyridin-4-yl)pyrazolo[1,5-a]pyridin-2-yl)cyclopropanecarboxamide (90 mg, 0.21 mmol) was purified purified by SFC over AD 250 mm×50 mm, 10 μm (eluent: 40% to 40% (v/v) supercritical CO₂ in EtOH and H₂O with 0.1% NH₃). The pure fractions were collected and the volatiles were removed under reduced pressure. The product was suspended in water (10 mL), the mixture frozen using dry ice/acetone, and then lyophilized to dryness to afford (*R)—N-[5-[5-[(3-methoxypyrrolidin-3-yl)methoxy]-2-methyl-4-pyridyl]pyrazolo[1,5-a]pyridin-2-yl]cyclopropanecarboxamide (32 mg, 35%). MS (ESI): mass calcd. for C₂₃H₂₇N₅O₃, 421.2. m/z found, 422.3 [M+H]⁺. ¹H NMR (400 MHz, CD₃OD) δ 8.42 (d, J=7.6 Hz, 1H), 8.28 (s, 1H), 7.81 (d, J=0.8 Hz, 1H), 7.39 (s, 1H), 7.06 (dd, J=2.0, 7.6 Hz, 1H), 6.90 (s, 1H), 4.37 (d, J=10.4 Hz, 1H), 4.24 (d, J=10.4 Hz, 1H), 3.23 (s, 3H), 3.14-3.05 (m, 2H), 3.04-2.96 (m, 1H), 2.96-2.89 (m, 1H), 2.53 (s, 3H), 2.14-2.03 (m, 1H), 1.92-1.79 (m, 2H), 1.03-0.95 (m, 2H), 0.93-0.85 (m, 2H).

### Example 215: (*S)—N-[5-[5-[(3-methoxypyrrolidin-3-yl)methoxy]-2-methyl-4-pyridyl]pyrazolo[1,5-a]pyridin-2-yl]cyclopropanecarboxamide

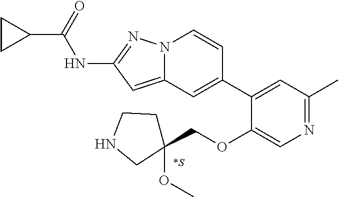 [SMILES: CO[C@@]1(COc2cnc(C)cc2-c2ccn3nc(NC(=O)C4CC4)cc3c2)CCNC1]

The title compound (35 mg, 38%) was prepared in Example 214, and is the other peak from the SFC purification step. MS (ESI): mass calcd. for C₂₃H₂₇N₅O₃, 421.2. m/z found, 422.3 [M+H]⁺. ¹H NMR (400 MHz, CD₃OD) δ 8.43 (d, J=7.6 Hz, 1H), 8.29 (s, 1H), 7.85-7.79 (m, 1H), 7.40 (s, 1H), 7.06 (dd, J=1.2, 7.2 Hz, 1H), 6.90 (s, 1H), 4.41 (d, J=10.4 Hz, 1H), 4.26 (d, J=10.4 Hz, 1H), 3.24 (s, 3H), 3.21-3.09 (m, 3H), 3.05-2.99 (m, 1H), 2.54 (s, 3H), 2.24-2.12 (m, 1H), 1.95-1.84 (m, 2H), 1.03-0.97 (m, 2H), 0.94-0.88 (m, 2H).

### Example 216: (*R)-3-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol

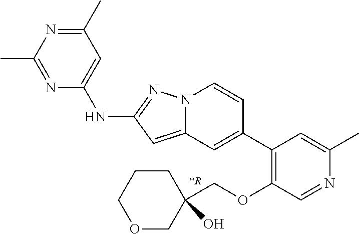

The title compound was prepared as described in Example 193, Step B, and using 1,5-dioxaspiro[2.5]octane instead of 2-cyclopropyl-2-methyloxirane; 3-(((4-(2-((2,6-dimethylpyrimidin-4-yl)amino)pyrazolo[1,5-a]pyridin-5-yl)-6-methylpyridin-3-yl)oxy)methyl)tetrahydro-2H-pyran-3-ol (140 mg, 0.304 mmol) was purified by SFC-17 over DAICEL CHIRALCEL OJ-H 250 mm×30 mm×5 μm (eluent: 35% to 35% (v/v) EtOH with 0.1% NH₃·H₂O in supercritical CO₂). The pure fractions were collected and the volatiles were removed under reduced pressure. The product was suspended in water (10 mL), the mixture frozen using dry ice/EtOH, and then lyophilized to dryness to afford (*R)-3-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol (47.20 mg, 33%) as a white solid. MS (ESI): mass calcd. for C₂₅H₂₈N₆O₃, 460.2. m/z found, 461.1 [M+H]⁺. ¹H NMR (400 MHz, CDCl₃) δ 8.31 (d, J=7.2 Hz, 1H), 8.28 (s, 1H), 7.58 (d, J=0.8 Hz, 1H), 7.54 (s, 1H), 7.16 (s, 1H), 7.02 (s, 1H), 6.85 (dd, J=2.0, 7.2 Hz, 1H), 6.57 (s, 1H), 4.11-4.06 (m, 1H), 4.05-4.01 (m, 1H), 3.81-3.73 (m, 1H), 3.68-3.61 (m, 2H), 3.61-3.52 (m, 1H), 3.01 (br. s., 1H), 2.57 (s, 3H), 2.56 (s, 3H), 2.45 (s, 3H), 1.97-1.85 (m, 1H), 1.81-1.69 (m, 2H), 1.61-1.54 (m, 1H).

### Example 217: 1-[[4-[2-[(2,6-dimethylpyrimidin-4-yl)amino]pyrazolo[1,5-a]pyridin-5-yl]-6-methyl-3-pyridyl]oxymethyl]cyclobutanol

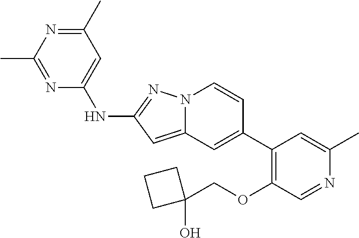

The title compound (33 mg, 22%) was prepared as described in Example 193, Step B, and using 1-oxaspiro[2.3]hexane instead of 2-cyclopropyl-2-methyloxirane and using Intermediate 16 instead of Intermediate 7. MS (ESI): mass calcd. for C₂₄H₂₆N₆O₂, 430.2. m/z found, 431.1 [M+H]⁺. ¹H NMR (400 MHz, CDCl₃) δ 8.34 (d, J=7.2 Hz, 1H), 8.31 (s, 1H), 7.63-7.58 (m, 1H), 7.43 (br. s., 1H), 7.18 (s, 1H), 7.05 (s, 1H), 6.92 (dd, J=1.2, 7.2 Hz, 1H), 6.55 (s, 1H), 4.10 (s, 2H), 2.59 (s, 3H), 2.57 (s, 3H), 2.44 (s, 3H), 2.21-2.08 (m, 4H), 1.90-1.78 (m, 1H), 1.62-1.54 (m, 1H).

### Example 218: (2R,3S)-2-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol

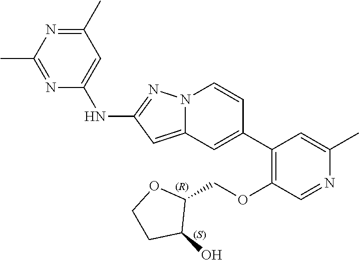

The title compound was prepared (7 mg, 7% yield) by the similar method of Example 188 by using Intermediate 16 instead of Intermediate 7 and ((2R,3S)-3-hydroxytetrahydrofuran-2-yl)methyl 4-methylbenzenesulfonate instead of (4,4-difluoro-1-hydroxycyclohexyl)methyl 4-methylbenzenesulfonate. MS (ESI): mass calcd. for C₂₄H₂₆N₆O₃, 446.2. m/z found, 447.1 [M+H]⁺. ¹H NMR (400 MHz, CDCl₃) δ 8.36 (d, J=7.2 Hz, 1H), 8.27 (s, 1H), 7.64 (s, 1H), 7.59 (s, 1H), 7.16 (s, 1H), 7.05 (s, 1H), 6.94 (dd, J=1.6, 7.2 Hz, 1H), 6.56 (s, 1H), 4.39-4.33 (m, 1H), 4.24-4.17 (m, 1H), 4.13-4.03 (m, 2H), 4.01-3.95 (m, 2H), 2.59 (s, 3H), 2.55 (s, 3H), 2.43 (s, 3H), 2.12-2.01 (m, 1H), 1.92-1.82 (m, 1H).

### Example 219: (*S)—N-(2,6-dimethylpyrimidin-4-yl)-5-[2-methyl-5-(8-oxaspiro[4.4]nonan-7-ylmethoxy)-4-pyridyl]pyrazolo[1,5-a]pyridin-2-amine

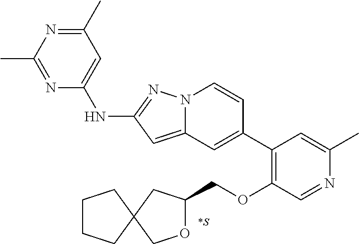

The title compound was prepared (7 mg, 7% yield) by the similar method of Example 188 by using Intermediate 16 instead of Intermediate 7 and ((2R,3S)-3-hydroxytetrahydrofuran-2-yl)methyl 4-methylbenzenesulfonate instead of (4,4-difluoro-1-hydroxycyclohexyl)methyl 4-methylbenzenesulfonate. Rac-5-(5-(2-oxaspiro[4.4]nonan-3-ylmethoxy)-2-methylpyridin-4-yl)-N-(2,6-dimethylpyrimidin-4-yl)pyrazolo[1,5-a]pyridin-2-amine (180 mg, 0.371 mmol) was purified by SFC-11 over DAICEL CHIRALPAK AD 250 mm×30 mm×10 μm (eluent: 45% to 45% (v/v) EtOH with 0.1% NH₃·H₂O in supercritical CO₂). The pure fractions were collected and the volatiles were removed under reduced pressure. The product was suspended in water (10 mL), the mixture frozen using dry ice/EtOH, and then lyophilized to dryness to afford fraction (*S)—N-(2,6-dimethylpyrimidin-4-yl)-5-[2-methyl-5-(8-oxaspiro[4.4]nonan-7-ylmethoxy)-4-pyridyl]pyrazolo[1,5-a]pyridin-2-amine 2 (74.1 mg, 41%) as second eluting peak. MS (ESI): mass calcd. for C₂₈H₃₂N₆O₂, 484.3. m/z found, 485.1 [M+H]⁺. ¹H NMR (400 MHz, CDCl₃) δ 8.36 (d, J=7.2 Hz, 1H), 8.29 (s, 1H), 7.68 (d, J=0.8 Hz, 1H), 7.42 (s, 1H), 7.18 (s, 1H), 7.07 (s, 1H), 7.00 (dd, J=2.0, 7.2 Hz, 1H), 6.57 (s, 1H), 4.36-4.29 (m, 1H), 4.12 (d, J=4.4 Hz, 2H), 3.62-3.57 (m, 2H), 2.60 (s, 3H), 2.56 (s, 3H), 2.45 (s, 3H), 1.93-1.84 (m, 1H), 1.76-1.72 (m, 1H), 1.66-1.49 (m, 8H).

### Example 220: (*S)-5-(5-((6-oxaspiro[3.4]octan-7-yl)methoxy)-2-methylpyridin-4-yl)-N-(2,6-dimethylpyrimidin-4-yl)pyrazolo[1,5-a]pyridin-2-amine

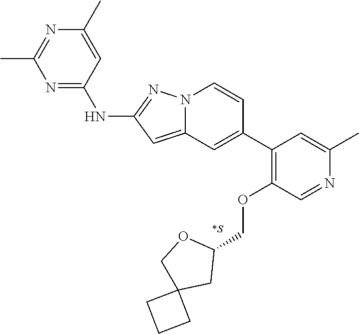

The title compound (78.7 mg, 22%) was prepared as described in Example 188 by using Intermediate 16 instead of Intermediate 7 and (rac-)-6-oxaspiro[3.4]octan-7-ylmethyl 4-methylbenzenesulfonate was used instead of (4,4-difluoro-1-hydroxycyclohexyl)methyl 4-methylbenzenesulfonate. The racemic material was purified by SFC-11 over DAICEL CHIRALPAK AD 250 mm×30 mm×10 μm (eluent: 45% to 45% (v/v) EtOH with 0.1% NH₃·H₂O in supercritical CO₂) to yield the title compound as a second eluting peak. MS (ESI): mass calcd. for C₂₇H₃₀N₆O₂, 470.2. m/z found, 471.2 [M+H]⁺. ¹H NMR (400 MHz, CDCl₃) δ 8.37 (d, J=7.2 Hz, 1H), 8.27 (s, 1H), 7.68 (d, J=0.8 Hz, 1H), 7.51 (s, 1H), 7.18 (s, 1H), 7.06 (s, 1H), 6.99 (dd, J=2.0, 7.2 Hz, 1H), 6.57 (s, 1H), 4.31-4.22 (m, 1H), 4.12-4.04 (m, 2H), 3.81-3.75 (m, 1H), 3.74-3.69 (m, 1H), 2.60 (s, 3H), 2.56 (s, 3H), 2.45 (s, 3H), 2.11-2.05 (m, 1H), 2.04-1.92 (m, 4H), 1.92-1.78 (m, 3H).

### Example 221: N-[5-[5-[(3-cyanoazetidin-3-yl)methoxy]-2-methyl-4-pyridyl]pyrazolo[1,5-a]pyridin-2-yl]cyclopropanecarboxamide

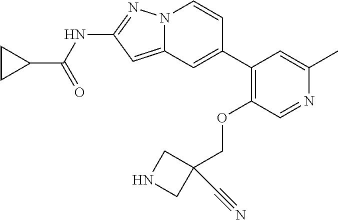 [SMILES: Cc1cc(-c2ccn3nc(NC(=O)C4CC4)cc3c2)c(OCC2(C#N)CNC2)cn1]

The title compound was prepared as a white solid (104 mg, 71% yield) by the similar method of Example 8 by using tert-butyl 3-(hydroxymethyl)-3-methoxyazetidine-1-carboxylate instead of (2S,4R)-tert-butyl 4-hydroxy-2-(trifluoromethyl)piperidine-1-carboxylate and Intermediate 6 instead of Intermediate 4. MS (ESI): mass calcd. for C₂₂H₂₂N₆O₂, 402.2. m/z found, 403.2 [M+H]⁺. ¹H NMR (400 MHz, CDCl₃) δ 8.51 (s, 1H), 8.32-8.26 (m, 2H), 7.64 (s, 1H), 7.22 (s, 1H), 7.02 (s, 1H), 7.01-6.97 (m, 1H), 4.46 (s, 2H), 4.05 (d, J=8.0 Hz, 2H), 3.56 (d, J=8.0 Hz, 2H), 2.58 (s, 3H), 1.64-1.52 (m, 1H), 1.19-1.11 (m, 2H), 0.98-0.85 (m, 2H).

### Example 222: (*S)—N-[5-[5-[(3-hydroxypyrrolidin-3-yl)methoxy]-2-methyl-4-pyridyl]pyrazolo[1,5-a]pyridin-2-yl]cyclopropanecarboxamide

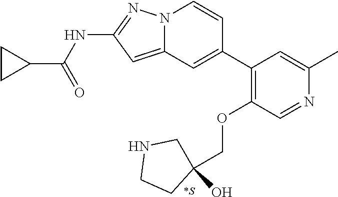

The title compound (46 mg, 7.1%) was prepared as described in Example 188 by using tert-butyl 3-hydroxy-3-((tosyloxy)methyl)pyrrolidine-1-carboxylate instead of (4,4-difluoro-1-hydroxycyclohexyl)methyl 4-methylbenzenesulfonate. The Boc protecting group was removed using 1:3 TFA:DCM, followed by an SFC purification step similar to that described in Example 5, to afford a white solid. MS (ESI): mass calcd. for C₂₂H₂₅N₅O₃, 407.2. m/z found, 408.2 [M+H]⁺. ¹H NMR (400 MHz, CDCl₃) δ 8.61 (br. s., 1H), 8.27 (s, 1H), 8.22 (d, J=7.6 Hz, 1H), 7.61 (s, 1H), 7.17 (s, 1H), 6.95 (s, 1H), 6.88 (dd, J=1.6, 7.2 Hz, 1H), 4.22-4.10 (m, 2H), 3.28-3.18 (m, 1H), 3.07-2.89 (m, 3H), 2.56 (s, 3H), 1.91-1.86 (m, 2H), 1.57-1.44 (m, 1H), 1.14-1.05 (m, 2H), 0.89-0.82 (m, 2H).

### Example 223: (*R)—N-[5-[5-[(3-hydroxypyrrolidin-3-yl)methoxy]-2-methyl-4-pyridyl]pyrazolo[1,5-a]pyridin-2-yl]cyclopropanecarboxamide

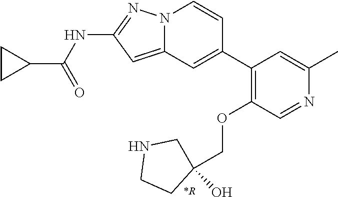

The title compound (12.6 mg, 1.76%) was prepared as described in Example 188 by using tert-butyl 3-hydroxy-3-((tosyloxy)methyl)pyrrolidine-1-carboxylate instead of (4,4-difluoro-1-hydroxycyclohexyl)methyl 4-methylbenzenesulfonate. The Boc protecting group was removed using 1:3 TFA:DCM, followed by an SFC purification step similar to that described in Example 5, to afford a white solid. MS (ESI): mass calcd. for C₂₂H₂₅N₅O₃, 407.2. m/z found, 408.1 [M+H]⁺. ¹H NMR (400 MHz, CDCl₃) δ 8.72 (br. s., 1H), 8.30-8.24 (m, 1H), 8.20 (d, J=6.4 Hz, 1H), 7.61 (s, 1H), 7.17 (s, 1H), 6.97-6.91 (m, 1H), 6.88 (d, J=7.2 Hz, 1H), 4.23-4.10 (m, 2H), 3.28-3.18 (m, 1H), 3.09-3.02 (m, 1H), 2.99-2.89 (m, 2H), 2.56 (s, 3H), 1.93-1.89 (m, 2H), 1.55-1.42 (m, 1H), 1.13-1.07 (m, 2H), 0.87-0.81 (m, 2H).

### Example 224: (1r,4r)-4-((4-(2-((2-ethyl-6-methylpyrimidin-4-yl)amino)pyrazolo[1,5-a]pyridin-5-yl)-6-methylpyridin-3-yl)oxy)cyclohexan-1-ol

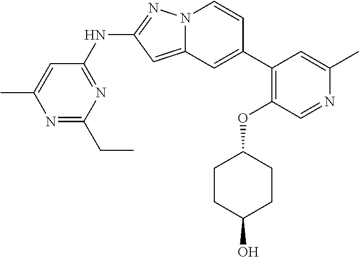

A vial containing a mixture of (1r,4r)-4-((4-(2-aminopyrazolo[1,5-a]pyridin-5-yl)-6-methylpyridin-3-yl)oxy)cyclohexan-1-ol (Intermediate 12, 100 mg, 0.296 mmol), 4-chloro-2-ethyl-6-methylpyrimidine (50.9 mg, 0.325 mmol), t-BuBRETTPHOS Pd G3 (12.6 mg, 0.0148 mmol), and Cs₂CO₃ (289 mg, 0.887 mmol) was purged with nitrogen using a vacuum line 3×. Then, 1,4-dioxane [123-91-1] (0.591 mL) was added via the septum. The mixture was cycled through nitrogen/vacuum 3×. Under vacuum, an argon balloon was added, and then the vacuum line was removed. The reaction was heated at 90° C. for 4 h. The crude was filtered through silica plug (rinsed with EtOAc, then DMSO/EtOAc). Concentrated, then purified over Basic ISCO (Gemini 5 uM NX-C18 110 angstrom LC column 150×21.2 mm; 10-100% AcN/20 mM aq NH₃ over 15 min; 40 mL/min). The pure fraction was collected and lyophilized to afford a white solid (54.6 mg, 40%). MS (ESI): mass calcd. for C₂₆H₃₀N₆O₂, 458.2. m/z found, 459.3 [M+H]⁺. ¹H NMR (400 MHz, DMSO-d6) δ 10.19 (s, 1H), 8.60-8.54 (m, 1H), 8.35 (s, 1H), 7.83-7.75 (m, 1H), 7.35 (s, 1H), 7.01 (dd, J=7.2, 2.0 Hz, 1H), 6.98 (s, 1H), 6.92 (s, 1H), 4.53 (d, J=3.8 Hz, 1H), 4.46-4.36 (m, 1H), 3.56-3.46 (m, 1H), 2.74 (q, J=7.6 Hz, 2H), 2.46 (s, 3H), 2.32 (s, 3H), 2.03-1.92 (m, 2H), 1.79-1.69 (m, 2H), 1.49-1.37 (m, 2H), 1.34-1.21 (m, 5H).

### Example 225: (1r,4r)-4-((4-(2-((2-(methoxymethyl)-6-methylpyrimidin-4-yl)amino)pyrazolo[1,5-a]pyridin-5-yl)-6-methylpyridin-3-yl)oxy)cyclohexan-1-ol

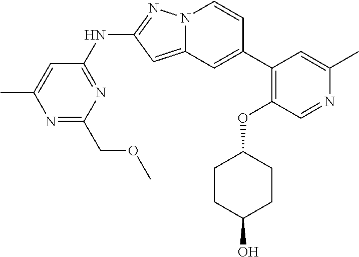

The title compound (43 mg, 29%) was prepared as described in Example 224 where 4-chloro-2-(methoxymethyl)-6-methylpyrimidine was used instead of 4-chloro-2-ethyl-6-methylpyrimidine. MS (ESI): mass calcd. for C₂₆H₃₀N₆O₃, 474.2. m/z found, 475.3 [M+H]⁺. ¹H NMR (400 MHz, DMSO-d6) δ 10.35 (s, 1H), 8.63-8.55 (m, 1H), 8.36 (s, 1H), 7.83-7.77 (m, 1H), 7.36 (s, 1H), 7.10 (s, 1H), 7.02 (dd, J=7.2, 2.0 Hz, 1H), 6.91 (s, 1H), 4.52 (d, J=4.0 Hz, 1H), 4.46-4.37 (m, 3H), 3.55-3.45 (m, 1H), 3.41 (s, 3H), 2.46 (s, 3H), 2.35 (s, 3H), 2.03-1.92 (m, 2H), 1.80-1.69 (m, 2H), 1.48-1.35 (m, 2H), 1.34-1.22 (m, 2H).

### Example 226: 6-((5-(5-(((1r,4r)-4-hydroxycyclohexyl)oxy)-2-methylpyridin-4-yl)pyrazolo[1,5-a]pyridin-2-yl)amino)-2,4-dimethylpyridazin-3(2H)-one

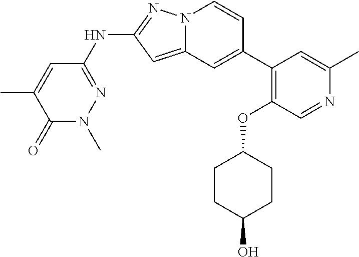

The title compound (88 mg, 62%) was prepared as described in Example 224 where 6-chloro-2,4-dimethylpyridazin-3(2h)-one was used instead of 4-chloro-2-ethyl-6-methylpyrimidine. MS (ESI): mass calcd. for C₂₅H₂₈N₆O₃, 460.2. m/z found, 461.2 [M+H]⁺. ¹H NMR (400 MHz, DMSO-d6) δ 9.67 (s, 1H), 8.45 (d, J=7.1 Hz, 1H), 8.28 (s, 1H), 7.76-7.61 (m, 1H), 7.27 (s, 1H), 7.20-7.13 (m, 1H), 6.93 (dd, J=7.2, 2.0 Hz, 1H), 6.80-6.74 (m, 1H), 4.45 (d, J=4.0 Hz, 1H), 4.39-4.28 (m, 1H), 3.55 (s, 3H), 3.48-3.36 (m, 1H), 2.39 (s, 3H), 2.05-1.98 (m, 3H), 1.95-1.86 (m, 2H), 1.71-1.62 (m, 2H), 1.40-1.28 (m, 2H), 1.27-1.14 (m, 2H).

### Example 227: (1r,4r)-4-((6-methyl-4-(2-((6-methyl-2-(methylamino)pyrimidin-4-yl)amino)pyrazolo[1,5-a]pyridin-5-yl)pyridin-3-yl)oxy)cyclohexan-1-ol

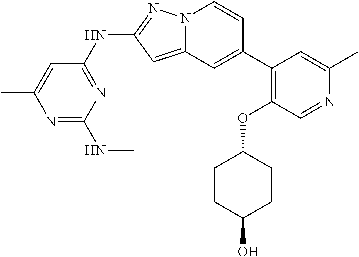

The title compound (72.4 mg, 35%) was prepared as described in Example 224 where 4-chloro-N,6-dimethylpyrimidin-2-amine was used instead of 4-chloro-2-ethyl-6-methylpyrimidine. MS (ESI): mass calcd. for C₂₅H₂₉N₇O₂, 459.2. m/z found, 460.3 [M+H]⁺. ¹H NMR (400 MHz, DMSO-d6) δ 9.82 (s, 1H), 8.57-8.48 (m, 1H), 8.35 (s, 1H), 7.73 (s, 1H), 7.35 (s, 1H), 7.05 (s, 1H), 6.98 (dd, J=7.2, 2.0 Hz, 1H), 6.63 (d, J=5.9 Hz, 1H), 6.22 (s, 1H), 4.51 (d, J=4.0 Hz, 1H), 4.45-4.35 (m, 1H), 3.56-3.43 (m, 1H), 2.85 (d, J=4.7 Hz, 3H), 2.46 (s, 3H), 2.14 (s, 3H), 2.03-1.90 (m, 2H), 1.80-1.69 (m, 2H), 1.47-1.35 (m, 2H), 1.34-1.21 (m, 2H).

### Example 228: 2-methyl-1-[[6-methyl-4-[2-[[6-methyl-2-(methylamino)pyrimidin-4-yl]amino]pyrazolo[1,5-a]pyridin-5-yl]-3-pyridyl]oxy]propan-2-ol

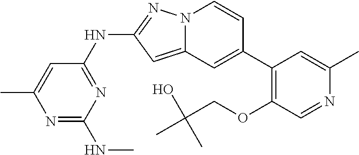

The title compound (24.4 mg, 16%) was prepared as described in Example 224 where 4-chloro-n,6-dimethylpyrimidin-2-amine was used instead of 4-chloro-2-ethyl-6-methylpyrimidine and 1-((4-(2-aminopyrazolo[1,5-a]pyridin-5-yl)-6-methylpyridin-3-yl)oxy)-2-methylpropan-2-ol (Intermediate 20) was used instead of (1r,4r)-4-((4-(2-aminopyrazolo[1,5-a]pyridin-5-yl)-6-methylpyridin-3-yl)oxy)cyclohexan-1-ol (Intermediate 12). MS (ESI): mass calcd. for C₂₃H₂₇N₇O₂, 433.2. m/z found, 434.3 [M+H]⁺. ¹H NMR (400 MHz, DMSO-d6) δ 9.83 (s, 1H), 8.57-8.49 (m, 1H), 8.31 (s, 1H), 7.82 (s, 1H), 7.37 (s, 1H), 7.07-7.00 (m, 2H), 6.67-6.53 (m, 1H), 6.22 (s, 1H), 4.62 (s, 1H), 3.87 (s, 2H), 2.84 (d, J=4.7 Hz, 3H), 2.46 (s, 3H), 2.13 (s, 3H), 1.15 (s, 6H).

### Example 229: 1-[[4-[2-[(2-cyclopropyl-6-methyl-pyrimidin-4-yl)amino]pyrazolo[1,5-a]pyridin-5-yl]-6-methyl-3-pyridyl]oxy]-2-methyl-propan-2-ol

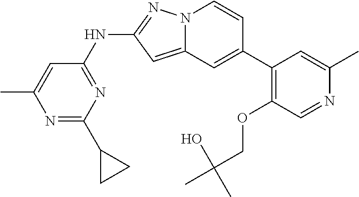

The title compound (52 mg, 36%) was prepared as described in Example 224 where 4-chloro-2-cyclopropyl-6-methylpyrimidine was used instead of 4-chloro-2-ethyl-6-methylpyrimidine and 1-((4-(2-aminopyrazolo[1,5-a]pyridin-5-yl)-6-methylpyridin-3-yl)oxy)-2-methylpropan-2-ol (Intermediate 20) was used instead of (1r,4r)-4-((4-(2-aminopyrazolo[1,5-a]pyridin-5-yl)-6-methylpyridin-3-yl)oxy)cyclohexan-1-ol (Intermediate 12). MS (ESI): mass calcd. for C₂₅H₂₈N₆O₂, 444.2. m/z found, 445.2 [M+H]⁺. ¹H NMR (400 MHz, DMSO-d6) δ 10.15 (s, 1H), 8.61-8.54 (m, 1H), 8.32 (s, 1H), 7.93-7.83 (m, 1H), 7.39 (s, 1H), 7.09 (dd, J=7.2, 2.0 Hz, 1H), 6.86 (s, 2H), 4.64 (s, 1H), 3.88 (s, 2H), 2.47 (s, 3H), 2.28 (s, 3H), 2.10-1.99 (m, 1H), 1.17 (s, 6H), 1.06-0.94 (m, 4H).

### Example 230: (*S)—N-(2,6-dimethylpyrimidin-4-yl)-5-[2-methyl-5-(9-oxaspiro[4.4]nonan-8-ylmethoxy)-4-pyridyl]pyrazolo[1,5-a]pyridin-2-amine

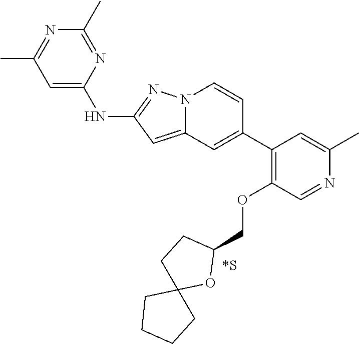

The title compound was prepared by the similar method of Example 188 by using Intermediate 16 instead of Intermediate 7 and 1-oxaspiro[4.4]nonan-2-ylmethyl 4-methylbenzenesulfonate instead of (4,4-difluoro-1-hydroxycyclohexyl)methyl 4-methylbenzenesulfonate; 5-(5-(1-oxaspiro[4.4]nonan-2-ylmethoxy)-2-methylpyridin-4-yl)-N-(2,6-dimethylpyrimidin-4-yl)pyrazolo[1,5-a]pyridin-2-amine (233 mg, 0.481 mmol) was purified by SFC-22 over DAICEL CHIRALPAK AD 250 mm×50 mm×10 μm (eluent: 50% to 50% (v/v) EtOH with 0.1% NH₃·H₂O in supercritical CO₂). The pure fractions were collected and the volatiles were removed under reduced pressure. The product was suspended in water (10 mL), the mixture frozen using dry ice/EtOH, and then lyophilized to dryness to afford fraction (*R)—N-(2,6-dimethylpyrimidin-4-yl)-5-[2-methyl-5-(9-oxaspiro[4.4]nonan-8-ylmethoxy)-4-pyridyl]pyrazolo[1,5-a]pyridin-2-amine (70.3 mg, 30%) and title compound (70.2 mg, 30%). MS (ESI): mass calcd. for C₂₈H₃₂N₆O₂, 484.3. m/z found, 485.2 [M+H]⁺. ¹H NMR (400 MHz, DMSO-d6) δ 10.21 (s, 1H), 8.57 (d, J=6.8 Hz, 1H), 8.34 (s, 1H), 7.92 (d, J=0.8 Hz 1H), 7.36 (s, 1H), 7.05 (dd, J=2.0, 7.2 Hz, 1H), 6.98 (br. s., 1H), 6.88 (br. s., 1H), 4.22-4.14 (m, 2H), 4.11-4.02 (m, 1H), 2.47 (s, 3H), 2.46 (s, 3H), 2.30 (s, 3H), 2.05-1.93 (m, 1H), 1.83-1.73 (m, 3H), 1.72-1.59 (m, 4H), 1.58-1.43 (m, 4H).

### Example 231: (*R)—N-(2,6-dimethylpyrimidin-4-yl)-5-[2-methyl-5-(9-oxaspiro[4.4]nonan-8-ylmethoxy)-4-pyridyl]pyrazolo[1,5-a]pyridin-2-amine

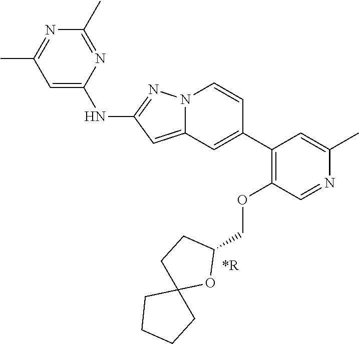

The title compound is the other isolated isomer from the SFC purification step in Example 230. MS (ESI): mass calcd. for C₂₈H₃₂N₆O₂, 484.3. m/z found, 485.2 [M+H]⁺. ¹H NMR (400 MHz, DMSO-d6) δ 10.21 (s, 1H), 8.57 (d, J=7.2 Hz, 1H), 8.34 (s, 1H), 7.92 (d, J=0.8 Hz, 1H), 7.36 (s, 1H), 7.05 (dd, J=2.0, 7.2 Hz, 1H), 6.98 (br. s., 1H), 6.88 (br. s., 1H), 4.22-4.14 (m, 2H), 4.09-4.02 (m, 1H), 2.47 (s, 3H), 2.46 (s, 3H), 2.30 (s, 3H), 2.03-1.93 (m, 1H), 1.85-1.73 (m, 3H), 1.72-1.59 (m, 4H), 1.57-1.43 (m, 4H).

### Example 232: (*S)—N-(2,6-dimethylpyrimidin-4-yl)-5-[2-methyl-5-[(2-methyltetrahydrofuran-2-yl)methoxy]-4-pyridyl]pyrazolo[1,5-a]pyridin-2-amine

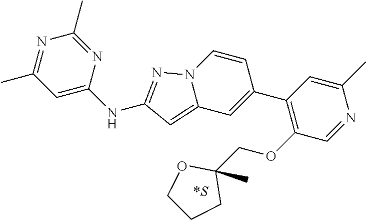

The title compound was prepared by the similar method of Example 188 and using Intermediate 16 instead of Intermediate 7 and using (2-methyltetrahydrofuran-2-yl)methyl 4-methylbenzenesulfonate instead of (4,4-difluoro-1-hydroxycyclohexyl)methyl 4-methylbenzenesulfonate. A subsequent SFC purification step, similar to that described in Example 5, yielded the title compound (15.1 mg, 3.2%). MS (ESI): mass calcd. for C₂₅H₂₈N₆O₂, 444.2. m/z found, 445.3 [M+H]⁺. ¹H NMR (400 MHz, DMSO-d6) δ 10.22 (s, 1H), 8.61 (d, J=7.2 Hz, 1H), 8.34 (s, 1H), 7.83 (d, J=0.8 Hz, 1H), 7.36 (s, 1H), 7.07-6.99 (m, 2H), 6.84 (s, 1H), 4.04-3.95 (m, 2H), 3.78-3.64 (m, 2H), 2.47 (s, 3H), 2.46 (s, 3H), 2.30 (s, 3H), 2.03-1.89 (m, 1H), 1.88-1.77 (m, 2H), 1.66-1.56 (m, 1H), 1.19 (s, 3H).

### Example 233: N-[5-[5-[(3-methoxyazetidin-3-yl)methoxy]-2-methyl-4-pyridyl]pyrazolo[1,5-a]pyridin-2-yl]cyclopropanecarboxamide

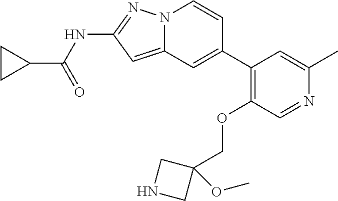 [SMILES: COC1(COc2cnc(C)cc2-c2ccn3nc(NC(=O)C4CC4)cc3c2)CNC1]

The title compound was prepared as a white solid (30 mg, 7.4%) by the similar method of Example 8 by using tert-butyl 3-(hydroxymethyl)-3-methoxyazetidine-1-carboxylate instead of (2S,4R)-tert-butyl 4-hydroxy-2-(trifluoromethyl)piperidine-1-carboxylate and Intermediate 6 instead of Intermediate 4. MS (ESI): mass calcd. for C₂₂H₂₅N₅O₃, 407.2. m/z found, 408.1 [M+H]⁺. ¹H NMR (400 MHz, DMSO-d6) δ 11.11 (s, 1H), 8.56 (d, J=7.2 Hz, 1H), 8.42 (s, 1H), 7.87 (s, 1H), 7.38 (s, 1H), 7.07 (dd, J=2.0, 7.2 Hz, 1H), 6.86 (s, 1H), 4.38 (s, 2H), 3.50 (d, J=8.4 Hz, 2H), 3.27 (d, J=8.4 Hz, 2H), 3.15 (s, 3H), 2.47 (s, 3H), 2.00-1.88 (m, 1H), 0.85-0.79 (m, 4H).

### Example 234: (1R,2R)-2-methyl-N-[5-[2-methyl-5-[[(1R,4R)-5-oxa-2-azabicyclo[2.2.1]heptan-4-yl]methoxy]-4-pyridyl]pyrazolo[1,5-a]pyridin-2-yl]cyclopropanecarboxamide

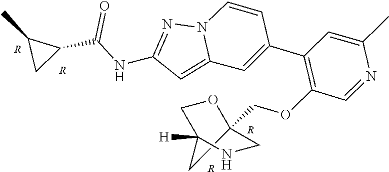

The title compound (224 mg, 78%) was prepared as described in Example 238 where (1R,2R)—N-(5-(5-fluoro-2-methylpyridin-4-yl)pyrazolo[1,5-a]pyridin-2-yl)-2-methylcyclopropane-1-carboxamide was used instead of N-(2,6-dimethylpyrimidin-4-yl)-5-(5-fluoro-2-methylpyridin-4-yl)pyrazolo[1,5-a]pyridin-2-amine. The Boc protecting group was removed using 10:1 TFA:DCM. The crude reaction was titurated with hexane/DCM to afford a white solid. MS (ESI): mass calcd. for C₂₄H₂₇N₅O₃, 433.2. m/z found, 434.3 [M+H]⁺. ¹H NMR (400 MHz, CD₃OD) δ 8.43-8.38 (m, 1H), 8.28 (s, 1H), 7.85-7.81 (m, 1H), 7.39 (s, 1H), 7.08 (dd, J=7.3, 2.0 Hz, 1H), 6.88 (s, 1H), 4.48 (s, 2H), 3.94-3.83 (m, 2H), 3.71 (s, 1H), 3.05-2.94 (m, 2H), 2.53 (s, 3H), 1.91-1.84 (m, 1H), 1.82-1.75 (m, 1H), 1.64-1.56 (m, 1H), 1.45-1.34 (m, 1H), 1.23-1.14 (m, 4H), 0.77-0.70 (m, 1H).

### Example 235: N-(6-cyclopropylpyridazin-3-yl)-5-[2-methyl-5-[[(1R,4R)-2-methyl-5-oxa-2-azabicyclo[2.2.1]heptan-4-yl]methoxy]-4-pyridyl]pyrazolo[1,5-a]pyridin-2-amine

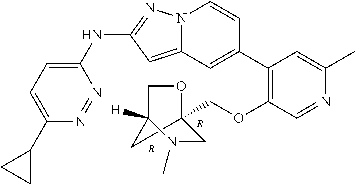

The title compound (10.3 mg, 36%) was prepared as described in Example 248 (Step A) and Example 249 (Step B). In Step A, N-(6-cyclopropylpyridazin-3-yl)-5-(5-fluoro-2-methylpyridin-4-yl)pyrazolo[1,5-a]pyridin-2-amine was used instead of N-(5-(5-fluoro-2-methylpyridin-4-yl)pyrazolo[1,5-a]pyridin-2-yl)cyclopropanecarboxamide. The Boc protecting group was removed using 10:1 TFA:DCM. The crude reaction was titurated with hexane/DCM to afford a white solid. The reductive methylation (Step B) was as described in Example 249. MS (ESI): mass calcd. for C₂₇H₂₉N₇O₂, 483.2. m/z found, 484.3 [M+H]⁺. ¹H NMR (400 MHz, CD₃OD) δ 8.46-8.39 (m, 1H), 8.26 (s, 1H), 7.83-7.77 (m, 1H), 7.65 (d, J=9.3 Hz, 1H), 7.40 (s, 1H), 7.29 (d, J=9.3 Hz, 1H), 7.03 (dd, J=7.2, 2.0 Hz, 1H), 6.85 (s, 1H), 4.45-4.38 (m, 2H), 4.15-4.11 (m, 1H), 3.77 (dd, J=8.1, 1.9 Hz, 1H), 3.49-3.45 (m, 1H), 2.90 (d, J=10.1 Hz, 1H), 2.67 (dd, J=10.2, 1.5 Hz, 1H), 2.53 (s, 3H), 2.41 (s, 3H), 2.21-2.16 (m, 1H), 1.99-1.93 (m, 1H), 1.85-1.79 (m, 1H), 1.11-1.05 (m, 2H), 1.01-0.94 (m, 2H).

### Example 236: 5-[2-methyl-5-[[(1R,4R)-2-methyl-5-oxa-2-azabicyclo[2.2.1]heptan-4-yl]methoxy]-4-pyridyl]-N-(5-methylpyrazin-2-yl)pyrazolo[1,5-a]pyridin-2-amine

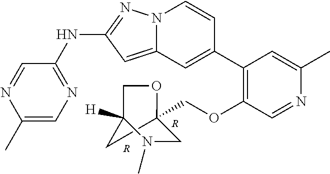

The title compound (11.7 mg, 31%) was prepared as described in Example 248 (Step A) and Example 249 (Step B). In Step A, 5-(5-fluoro-2-methylpyridin-4-yl)-N-(5-methylpyrazin-2-yl)pyrazolo[1,5-a]pyridin-2-amine was used instead of N-(5-(5-fluoro-2-methylpyridin-4-yl)pyrazolo[1,5-a]pyridin-2-yl)cyclopropanecarboxamide. The Boc protecting group was removed using 10:1 TFA:DCM. The crude reaction was titurated with hexane/DCM to afford a white solid. The reductive methylation (Step B) was as described in Example 249. MS (ESI): mass calcd. for C₂₅H₂₇N₇O₂, 457.2. m/z found, 458.2 [M+H]⁺. ¹H NMR (400 MHz, CD₃OD) δ 8.55 (d, J=1.5 Hz, 1H), 8.45-8.41 (m, 1H), 8.26 (s, 1H), 8.15-8.11 (m, 1H), 7.80-7.76 (m, 1H), 7.39 (s, 1H), 7.02 (dd, J=7.3, 2.0 Hz, 1H), 6.80 (d, J=0.9 Hz, 1H), 4.43-4.39 (m, 2H), 4.14-4.10 (m, 1H), 3.76 (dd, J=8.1, 1.9 Hz, 1H), 3.47 (s, 1H), 2.93-2.88 (m, 1H), 2.67 (dd, J=10.2, 1.6 Hz, 1H), 2.53 (s, 3H), 2.44 (s, 3H), 2.41 (s, 3H), 1.99-1.94 (m, 1H), 1.84-1.79 (m, 1H).

### Example 237: N-[5-[2-methyl-5-[[(1S,4S)-2-methyl-5-oxa-2-azabicyclo[2.2.1]heptan-4-yl]methoxy]-4-pyridyl]pyrazolo[1,5-a]pyridin-2-yl]cyclopropanecarboxamide

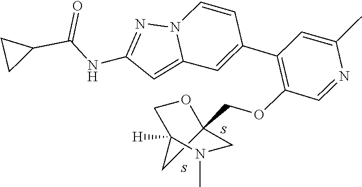

The title compound was obtained using the reductive methylation conditions described in Example 249, and using Example 253 instead of N-[5-[2-methyl-5-[[(1R,4R)-5-oxa-2-azabicyclo[2.2.1]heptan-4-yl]methoxy]-4-pyridyl]pyrazolo[1,5-a]pyridin-2-yl]cyclopropanecarboxamide. MS (ESI): mass calcd. for C₂₄H₂₇N₅O₃, 433.2. m/z found, 434.2 [M+H]⁺. ¹H NMR (400 MHz, CD₃OD) δ 8.44-8.40 (m, 1H), 8.26 (s, 1H), 7.84-7.81 (m, 1H), 7.38 (s, 1H), 7.07 (dd, J=7.3, 2.0 Hz, 1H), 6.93-6.87 (m, 1H), 4.45-4.37 (m, 2H), 4.11 (dd, J=8.1, 1.1 Hz, 1H), 3.76 (dd, J=8.1, 1.9 Hz, 1H), 3.49-3.45 (m, 1H), 2.90 (d, J=10.1 Hz, 1H), 2.66 (dd, J=10.2, 1.5 Hz, 1H), 2.53 (s, 3H), 2.41 (s, 3H), 1.98-1.93 (m, 1H), 1.91-1.83 (m, 1H), 1.82-1.78 (m, 1H), 1.02-0.97 (m, 2H), 0.93-0.86 (m, 2H).

### Example 238: N-(2,6-dimethylpyrimidin-4-yl)-5-[2-methyl-5-[[(1R,4R)-5-oxa-2-azabicyclo[2.2.1]heptan-4-yl]methoxy]-4-pyridyl]pyrazolo[1,5-a]pyridin-2-amine

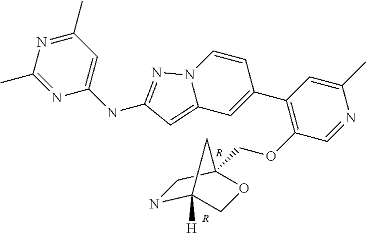

To a scintillation vial containing a stir bar was added N-(2,6-dimethylpyrimidin-4-yl)-5-(5-fluoro-2-methylpyridin-4-yl)pyrazolo[1,5-a]pyridin-2-amine (Intermediate 15, 100 mg, 0.287 mmol), tert-butyl (1R,4R)-1-(hydroxymethyl)-2-oxa-5-azabicyclo[2.2.1]heptane-5-carboxylate (98.7 mg, 1.431 mmol) and DMA (2 mL). NaH (60% dispersion in mineral oil) (68.8 mg, 1.72 mmol) was added and the reaction mixture was stirred at room temperature for 2 minutes before placing the vial on a pre-heated block at 90° C. The reaction mixture was heated for 1 h, cooled to RT, extracted with water (20 mL) and 10% MeOH/DCM (2×50 mL). The combined organic layers were dried (MgSO₄) and concentrated. The residue was treated with 1:1 trifluoroacetic acid (2 mL):DCM (2 mL) and the reaction mixture was stirred at room-temperature for 1 h. The reaction mixture was concentrated to dryness under reduced pressure to afford the crude product, then purified by reverse phase basic HPLC (H₂O, CH₃CN, NH₄OH). Fractions containing the title compound were combined, concentrated and lyophilized from 1:1 CH₃CN:water to provide the title compound as a white solid (104 mg, 79%). MS (ESI): mass calcd. for C₂₅H₂₇N₇O₂, 457.5. m/z found, 458.2 [M+H]⁺. ¹H NMR (500 MHz, DMSO-d6) δ 10.12 (s, 1H), 8.52 (d, J=7.2 Hz, 1H), 8.30 (s, 1H), 7.83-7.75 (m, 1H), 7.31 (s, 1H), 6.99 (dd, J=7.2, 2.0 Hz, 1H), 6.96 (s, 1H), 6.78 (s, 1H), 4.39 (s, 2H), 3.72 (dd, J=6.9, 1.8 Hz, 1H), 3.62 (d, J=6.9 Hz, 1H), 3.49-3.44 (m, 1H), 2.86-2.72 (m, 2H), 2.39 (d, J=6.4 Hz, 6H), 2.23 (s, 3H), 1.63-1.52 (m, 2H).

### Example 239: 4-[[6-methyl-4-[2-(1H-pyrrolo[2,3-b]pyridin-6-ylamino)pyrazolo[1,5-a]pyridin-5-yl]-3-pyridyl]oxy]cyclohexanol

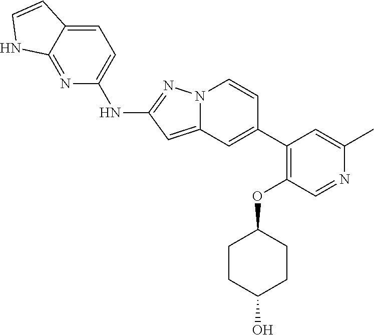 [SMILES: Cc1cc(-c2ccn3nc(Nc4ccc5cc[nH]c5n4)cc3c2)c(O[C@H]2CC[C@H](O)CC2)cn1]

A mixture of (1r,4r)-4-((4-(2-aminopyrazolo[1,5-a]pyridin-5-yl)-6-methylpyridin-3-yl)oxy)cyclohexan-1-ol (Intermediate 12) (105 mg, 0.310 mmol), 6-bromo-1H-pyrrolo[2,3-b]pyridine (67.2 mg, 0.341 mmol), t-BuBrettPhos Pd G3 (13.3 mg, 0.016 mmol), and Cs₂CO₃ (303 mg, 0.931 mmol) was placed in a Biotage vial: The capped vial was then evacuated and back-filled with N₂. The vial was evacuated again, and then an argon balloon was inserted and the solvent dioxane (0.6 mL) was added. The mixture was heated at 90° C. for 16 hr. The mixture was cooled down, solids were filtered off and rinsed with DMSO. The filtrate was purified over Basic ISCO (Gemini 5 uM NX-C18 110 angstrom LC column 150×21.2 mm; 10-100% CH₃CN/20 mM aq NH₃ over 15 min; 40 mL/min) and lyophilized providing the title compound (75.7 mg, 53.7%). MS (ESI): mass calcd. for C₂₆H₂₆N₆O₂, 454.5. m/z found, 455.2 [M+H]⁺. ¹H NMR (500 MHz, DMSO-d6) δ 11.12 (s, 1H), 9.58 (s, 1H), 8.45 (d, J=7.1, 0.9 Hz, 1H), 8.29 (s, 1H), 7.71 (dd, J=8.4, 0.7 Hz, 1H), 7.67-7.62 (m, 1H), 7.28 (s, 1H), 7.08-7.01 (m, 2H), 6.93-6.82 (m, 2H), 6.25-6.19 (m, 1H), 4.45 (d, J=4.0 Hz, 1H), 4.39-4.31 (m, 1H), 3.48-3.39 (m, 1H), 2.40 (s, 3H), 1.96-1.87 (m, 2H), 1.72-1.63 (m, 2H), 1.40-1.29 (m, 2H), 1.27-1.17 (m, 2H).

### Example 240: 2-methyl-1-[[6-methyl-4-[2-(1H-pyrrolo[2,3-b]pyridin-6-ylamino)pyrazolo[1,5-a]pyridin-5-yl]-3-pyridyl]oxy]propan-2-ol

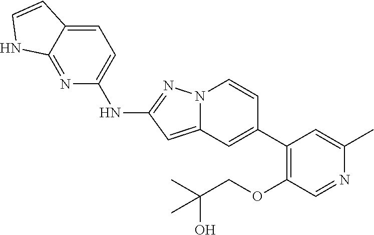 [SMILES: Cc1cc(-c2ccn3nc(Nc4ccc5cc[nH]c5n4)cc3c2)c(OCC(C)(C)O)cn1]

A mixture of 1-((4-(2-aminopyrazolo[1,5-a]pyridin-5-yl)-6-methylpyridin-3-yl)oxy)-2-methylpropan-2-ol (102 mg, 0.327 mmol), 6-bromo-1H-pyrrolo[2,3-b]pyridine (70.7 mg, 0.359 mmol), t-BuBrettPhos Pd G3 (14.0 mg, 0.016 mmol), and Cs₂CO₃ (319 mg, 0.980 mmol) was placed in a Biotage vial: The capped vial was then evacuated and back-filled with N₂. The vial was evacuated again, and then an argon balloon was inserted and the solvent dioxane (0.6 mL) was added. The mixture was heated at 90° C. for 16 hr. The mixture was cooled down, solids were filtered off and rinsed with DMSO. The filtrate was purified over Basic ISCO (Gemini 5 uM NX-C18 110 angstrom LC column 150×21.2 mm; 10-100% AcN/20 mM aq NH₃ over 15 min; 40 mL/min) and lyophilized providing the title compound (51.9 mg, 37.0%). MS (ESI): mass calcd. for C₂₄H₂₄N₆O₂, 428.5. m/z found, 429.2 [M+H]⁺. ¹H NMR (500 MHz, DMSO-d6) δ 11.25-11.16 (m, 1H), 9.65 (s, 1H), 8.56-8.49 (m, 1H), 8.32 (s, 1H), 7.80-7.74 (m, 2H), 7.37 (s, 1H), 7.13-7.10 (m, 1H), 7.09-7.07 (m, 1H), 7.00 (dd, J=7.2, 2.0 Hz, 1H), 6.97 (d, J=8.5 Hz, 1H), 6.30 (dd, J=3.4, 1.9 Hz, 1H), 4.63 (s, 1H), 3.88 (s, 2H), 2.47 (s, 3H), 1.17 (s, 6H).

### Example 241: 4-[[5-[5-(4-hydroxycyclohexoxy)-2-methyl-4-pyridyl]pyrazolo[1,5-a]pyridin-2-yl]amino]-2,6-dimethoxy-N-(2,2,2-trifluoroethyl)benzamide

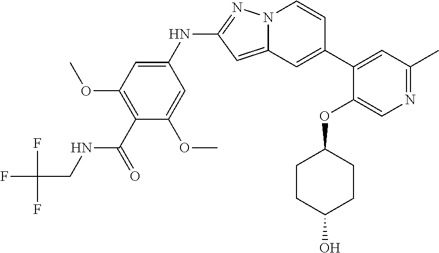 [SMILES: COc1cc(Nc2cc3cc(-c4cc(C)ncc4O[C@H]4CC[C@H](O)CC4)ccn3n2)cc(OC)c1C(=O)NCC(F)(F)F]

Step A. methyl 4-((5-(5-(((1r,4r)-4-hydroxycyclohexyl)oxy)-2-methylpyridin-4-yl)pyrazolo[1,5-a]pyridin-2-yl)amino)-2,6-dimethoxybenzoate. A mixture of (1r,4r)-4-((4-(2-aminopyrazolo[1,5-a]pyridin-5-yl)-6-methylpyridin-3-yl)oxy)cyclohexan-1-ol (Intermediate 12) (500 mg, 1.48 mmol), methyl 4-bromo-2,6-dimethoxybenzoate (488 mg, 1.77 mmol), BrettPhos Pd G3 (134 mg, 0.148 mmol), and Cs₂CO₃ (1.44 g, 0.443 mmol) was placed in a Biotage vial. The capped vial was then evacuated and back-filled with N₂. The vial was evacuated again, and then an argon balloon was inserted and the solvent dioxane (15 mL) was added. The mixture was heated at 90° C. for 4 hr. The mixture was cooled down, and solids were filtered off. The crude was diluted with brine and EtOAc. The organic layer was separated. and concentrated under vacuo. The residue was flashed with 10% MeOH/CH₂Cl₂ providing the title compound (499 mg, 63.4%). MS (ESI): mass calcd. for C₂₉H₃₂N₄O6, 532.2. m/z found, 533.2 [M+H]⁺.

Step B: 4-[[5-[5-(4-hydroxycyclohexoxy)-2-methyl-4-pyridyl]pyrazolo[1,5-a]pyridin-2-yl]amino]-2,6-dimethoxy-N-(2,2,2-trifluoroethyl)benzamide A solution of iPrMgCl (2M, 0.2 mL, 0.39 mmol) was added dropwisely to a stirred mixture of methyl 4-((5-(5-4(1r,4r)-4-hydroxycyclohexyl)oxy)-2-methylpyridin-4-yl)pyrazolo[1,5-a]pyridin-2-yl)amino)-2,6-dimethoxybenzoate (70 mg, 0.13 mmol), 2,2,2-trifluoroethan-1-amine (0.19 mL, 0.39 mmol) in THF (8 mL), The mixture was stirred at room temperature for 1 h. The reaction was quenched with sat. aq. NH₄Cl (3 mL) and extracted with EtOAc (3×10 mL). The combined organics were dried with Na₂SO₄, filtered and concentrated. The residue was then purified by preparative TLC with 0-5% MeOH in DCM, to provide the title compound (56 mg, 71%) MS (ESI): mass calcd. for C₃₀H₃₂F₃N₅O₅, 599.6. m/z found, 600.2 [M+H]⁺. ¹H NMR (500 MHz, Methanol-d4) δ 8.25 (d, J=7.2 Hz, 1H), 8.09 (s, 1H), 7.49 (dd, J=1.9, 0.8 Hz, 1H), 7.19 (s, 1H), 6.84-6.78 (m, 1H), 6.74 (s, 2H), 6.04 (s, 1H), 4.29-4.19 (m, 1H), 3.71 (s, 6H), 3.54-3.49 (m, 1H), 3.47-3.39 (m, 1H), 2.39 (s, 3H), 1.98-1.88 (m, 2H), 1.84-1.72 (m, 2H), 1.43-1.31 (m, 2H), 1.32-1.24 (m, 2H).

### Example 242: N-cyclopropyl-4-[[5-[5-(4-hydroxycyclohexoxy)-2-methyl-4-pyridyl]pyrazolo[1,5-a]pyridin-2-yl]amino]-2-methoxy-benzamide

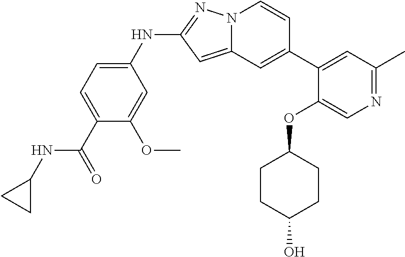 [SMILES: COc1cc(Nc2cc3cc(-c4cc(C)ncc4O[C@H]4CC[C@H](O)CC4)ccn3n2)ccc1C(=O)NC1CC1]

A solution of iPrMgCl (2M, 0.52 mL) was added dropwisely to a stirred mixture of methyl 4-[[5-[5-(4-hydroxycyclohexoxy)-2-methyl-4-pyridyl]pyrazolo[1,5-a]pyridin-2-yl]amino]-2-methoxy-benzoate (Example 246) (87 mg, 0.17 mmol), cyclopropyl amine (49 mg, 0.86 mmol) in THF (8 mL). The mixture was stirred at RT for 1 h. The reaction was quenched with sat. aq. NH₄Cl (3 mL) and extracted with EtOAc (3×10 mL). The combined organics were dried with Na₂SO₄, filtered and concentrated. The residue was then purified by preparative TLC with 0-5% MeOH in DCM, to provide the title compound (47 mg, 52%) MS (ESI): mass calcd. for C₃₀H₃₃N₅O₄, 527.6. m/z found, 528.3 [M+H]⁺. ¹H NMR (500 MHz, Methanol-d4) δ 8.27 (d, J=7.1 Hz, 1H), 8.10 (s, 1H), 7.78 (d, J=8.6 Hz, 1H), 7.50 (d, J=1.8 Hz, 1H), 7.31 (d, J=2.0 Hz, 1H), 7.20 (s, 1H), 6.92 (dd, J=8.6, 1.9 Hz, 1H), 6.80 (dd, J=7.1, 1.8 Hz, 1H), 6.06 (s, 1H), 4.32-4.17 (m, 1H), 3.87 (s, 3H), 3.58-3.46 (m, 1H), 2.77-2.67 (m, 1H), 2.40 (s, 3H), 2.00-1.91 (m, 2H), 1.83-1.67 (m, 2H), 1.45-1.32 (m, 2H), 1.32-1.19 (m, 2H), 0.75-0.64 (m, 2H), 0.54-0.38 (m, 2H).

### Example 243: N-ethyl-4-[[5-[5-(4-hydroxycyclohexoxy)-2-methyl-4-pyridyl]pyrazolo[1,5-a]pyridin-2-yl]amino]-2,6-dimethoxy-benzamide

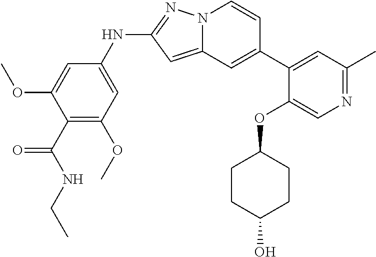

Step A: methyl 4-((5-(5-4(1r,4r)-4-hydroxycyclohexyl)oxy)-2-methylpyridin-4-yl)pyrazolo[1,5-a]pyridin-2-yl)amino)-2,6-dimethoxybenzoate. A mixture of (1r,4r)-4-((4-(2-aminopyrazolo[1,5-a]pyridin-5-yl)-6-methylpyridin-3-yl)oxy)cyclohexan-1-ol (Intermediate 12) (500 mg, 1.48 mmol), methyl 4-bromo-2,6-dimethoxybenzoate (488 mg, 1.77 mmol), BrettPhos Pd G3 (134 mg, 0.148 mmol), and Cs₂CO₃ (1.44 g, 0.443 mmol) was placed in a Biotage vial: The capped vial was then evacuated and back-filled with N₂. The vial was evacuated again, and then an argon balloon was inserted and the solvent dioxane (15 mL) was added. The mixture was heated at 90° C. for 4 hr. The mixture was cooled down, and solids were filtered off. The crude product was diluted with brine and EtOAc. The organic layer was separated. and concentrated under vacuo. The residue was flashed with 10% MeOH/CH₂Cl₂ providing the title compound (499 mg, 63.4%). MS (ESI): mass calcd. for C₂₉H₃₂N₄O6, 532.2. m/z found, 533.2 [M+H]⁺.

Step B: 4-[[5-[5-(4-hydroxycyclohexoxy)-2-methyl-4-pyridyl]pyrazolo[1,5-a]pyridin-2-yl]amino]-2,6-dimethoxy-N-(2,2,2-trifluoroethyl)benzamide. A solution of iPrMgCl (2M, 0.18 mL, 0.37 mmol) was added dropwise to a stirred mixture of methyl 4-((5-(5-4(1r,4r)-4-hydroxycyclohexyl)oxy)-2-methylpyridin-4-yl)pyrazolo[1,5-a]pyridin-2-yl)amino)-2,6-dimethoxybenzoate (65 mg, 0.12 mmol), and ethyl amine (2M, 0.3 mL, 0.6 mmol) in THF (8 mL), The mixture was stirred at RT for 1 h. The reaction was quenched with sat. aq. NH₄Cl (3 mL) and extracted with EtOAc (3×10 mL). The combined organics were dried with Na₂SO₄, filtered and concentrated. The residue was then purified by preparative TLC with 0-5% MeOH in DCM, to provide the title compound (36 mg, 54%) MS (ESI): mass calcd. for C₃₀H₃₅N₅O₅, 545.6. m/z found, 546.3 [M+H]⁺. ¹H NMR (500 MHz, Methanol-d4) δ 8.27 (d, J=7.2 Hz, 1H), 8.12 (s, 1H), 7.55-7.45 (m, 1H), 7.23 (s, 1H), 6.84-6.78 (m, 1H), 6.74 (s, 2H), 6.06 (s, 1H), 4.33-4.19 (m, 1H), 3.72 (s, 6H), 3.56-3.48 (m, 1H), 3.28-3.22 (m, 2H), 2.41 (s, 3H), 2.02-1.91 (m, 2H), 1.77-1.68 (m, 2H), 1.46-1.36 (m, 2H), 1.32-1.24 (m, 2H), 1.09 (t, J=7.3 Hz, 3H).

### Example 244: 6-[[5-[5-(2-hydroxy-2-methyl-propoxy)-2-methyl-4-pyridyl]pyrazolo[1,5-a]pyridin-2-yl]amino]-N,N,4-trimethyl-pyridine-2-carboxamide

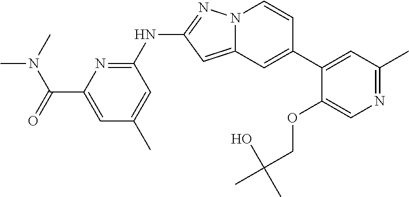

Step A. 6-chloro-N,N,4-trimethylpicolinamide. To a 0° C. solution of 6-chloro-4-methylpicolinic acid (858 g, 5.00 mmol) in DCM (13 mL) were added Et₃N (2.08 mL, 15.0 mmol), ethylamine (270 mg, 6.00 mmol) and HATU (2.47 g, 6.50 mmol). The mixture was stirred for 2 h. The mixture was partioned between DCM/NaHCO₃ saturated solution (20 mL/10 mL). The organic layer was separated and concentrated providing the crude product as the solid. The crude product was washed with CH₃CN (5 mL) providing the crude title compound (610 mg, 61.4%). MS (ESI): mass calcd. for C₉H₁₁ClN₂O, 198.06. m/z found, 199.1 [M+H]⁺.

Step B. 6-[[5-[5-(2-hydroxy-2-methyl-propoxy)-2-methyl-4-pyridyl]pyrazolo[1,5-a]pyridin-2-yl]amino]-N,N,4-trimethyl-pyridine-2-carboxamide. The mixture of (1-((4-(2-aminopyrazolo[1,5-a]pyridin-5-yl)-6-methylpyridin-3-yl)oxy)-2-methylpropan-2-ol (100 mg, 0.320 mmol), 6-chloro-N,N,4-trimethylpicolinamide (70 mg, 0.35 mmol), BrettPhos Pd G3 (29 mg, 0.032 mmol), and Cs₂CO₃ (312 mg, 0.960 mmol) was placed in a Biotage vial. The capped vial was then evacuated and back-filled with N₂. The vial was evacuated again, and then an argon balloon was inserted and the solvent dioxane (5 mL) was added. The mixture was heated at 90° C. for 2 hr. The mixture was cooled down, and solids were filtered off. The crude product was diluted with brine and EtOAc. The organic layer was separated. and concentrated under vacuo. The residue was flashed with 10% MeOH/CH₂Cl₂ providing the title compound (51 mg, 34%). MS (ESI): mass calcd. for C₂₆H₃₀N₆O₃, 474.6. m/z found, 475.2 [M+H]⁺. ¹H NMR (500 MHz, Methanol-d4) δ 8.30-8.20 (m, 1H), 8.08 (s, 1H), 7.62 (dd, J=2.0, 0.9 Hz, 1H), 7.20 (s, 1H), 7.04 (t, J=1.1 Hz, 1H), 6.87 (dd, J=7.2, 2.0 Hz, 1H), 6.75-6.64 (m, 2H), 3.79 (s, 2H), 3.03-2.96 (2 s, 6H), 2.38 (s, 3H), 2.22 (s, 3H), 1.14 (s, 6H).

### Example 245: N-cyclopropyl-4-[[5-[5-(4-hydroxycyclohexoxy)-2-methyl-4-pyridyl]pyrazolo[1,5-a]pyridin-2-yl]amino]-2,6-dimethoxy-benzamide

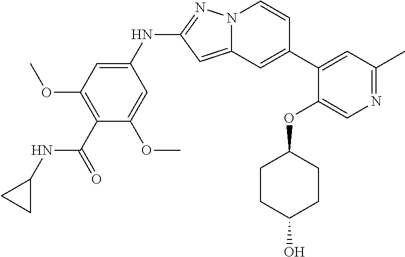 [SMILES: COc1cc(Nc2cc3cc(-c4cc(C)ncc4O[C@H]4CC[C@H](O)CC4)ccn3n2)cc(OC)c1C(=O)NC1CC1]

Step A. 4-bromo-N-cyclopropyl-2,6-dimethoxybenzamide. To a 0° C. solution of 4-bromo-2,6-dimethoxybenzoic acid (1.08 g, 4.13 mmol) in DCM (13 mL) were added Et₃N (1.72 mL, 12.4 mmol), cyclopropyl amine (283 mg, 4.96 mmol) and HATU (2.04 g, 5.38 mmol). The mixture was stirred for 2 h. The mixture was partioned between DCM/NaHCO₃ saturated solution (20 mL/10 mL). The organic layer was separated and concentrated providing the crude product as the solid. The crude product was washed with CH₃CN (5 mL) providing the title compound (1.10 g, 88.6%). MS (ESI): mass calcd. for C₁₂H₁₄BrNO₃, 299.01. m/z found, 300.0 [M+H]⁺. 1H NMR (500 MHz, Chloroform-d) δ 6.69 (s, 2H), 5.77 (s, 1H), 3.79 (s, 6H), 2.96-2.84 (m, 1H), 0.88-0.76 (m, 2H), 0.64-0.54 (m, 2H).

Step B. N-ethyl-4-[[5-[5-(4-hydroxycyclohexoxy)-2-methyl-4-pyridyl]pyrazolo[1,5-a]pyridin-2-yl]amino]-2,6-dimethoxy-benzamide. A mixture of (1r,4r)-4-((4-(2-aminopyrazolo[1,5-a]pyridin-5-yl)-6-methylpyridin-3-yl)oxy)cyclohexan-1-ol (Intermediate 12) (100 mg, 0.296 mmol), 4-bromo-N-cyclopropyl-2,6-dimethoxybenzamide (88.7 mg, 0.296 mmol), BrettPhos Pd G3 (15.3 mg, 0.018 mmol), and Cs₂CO₃ (176 mg, 0.541 mmol) was in a Biotage vial: The capped vial was then evacuated and back-filled with N₂. The vial was evacuated again, and then an argon balloon was inserted and the solvent dioxane (5 mL) was added. The mixture was heated at 90° C. for 2 hr. The mixture was cooled down, and solids were filtered off. The crude product was diluted with brine and EtOAc. The organic layer was separated. and concentrated under vacuo. The residue was flashed with 10% MeOH/CH₂Cl₂ providing the title compound (67 mg, 41%). MS (ESI): mass calcd. for C₃₁H₃₅N₅O₅, 557.6. m/z found, 558.2 [M+H]⁺. ¹H NMR (500 MHz, Methanol-d4) δ 8.31-8.18 (m, 1H), 8.09 (s, 1H), 7.54-7.43 (m, 1H), 7.19 (s, 1H), 6.83-6.77 (m, 1H), 6.75 (d, J=23.6 Hz, 2H), 6.03 (d, J=0.8 Hz, 1H), 4.34-4.10 (m, 1H), 3.70 (s, 6H), 3.55-3.47 (m, 1H), 2.76-2.65 (m, 1H), 2.39 (s, 3H), 1.97-1.87 (m, 2H), 1.78-1.69 (m, 2H), 1.45-1.33 (m, 2H), 1.29-1.19 (m, 2H), 0.72-0.61 (m, 2H), 0.52-0.44 (m, 2H).

### Example 246: methyl 4-[[5-[5-(4-hydroxycyclohexoxy)-2-methyl-4-pyridyl]pyrazolo[1,5-a]pyridin-2-yl]amino]-2-methoxy-benzoate

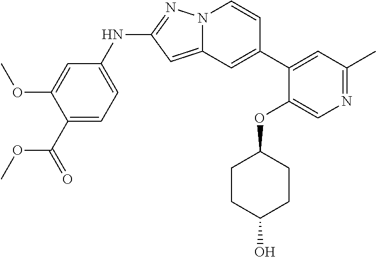

A mixture of (1r,4r)-4-((4-(2-aminopyrazolo[1,5-a]pyridin-5-yl)-6-methylpyridin-3-yl)oxy)cyclohexan-1-ol (Intermediate 12) (96.0 mg, 0.284 mmol), methyl 4-bromo-2-methoxybenzoate (76.5 mg, 0.312 mmol), BrettPhos Pd G3 (25.7 mg, 0.028 mmol), and Cs₂CO₃ (277 mg, 0.851 mmol) was placed in a Biotage vial: The capped vial was then evacuated and back-filled with N₂. The vial was evacuated again, and then an argon balloon was inserted and the solvent dioxane (5 mL) was added. The mixture was heated at 90° C. for 4 hr. The mixture was cooled down, and solids were filtered off. The crude product was diluted with brine and EtOAc. The organic layer was separated. and concentrated under vacuo. The residue was flashed with 10% MeOH/CH₂Cl₂ providing the title compound (90 mg, 63%). MS (ESI): mass calcd. for C₂₈H₃₀N₄O₅, 502.6. m/z found, 503.2 [M+H]⁺. ¹H NMR (500 MHz, Methanol-d4) δ 8.35-8.25 (m, 1H), 8.13 (s, 1H), 7.68 (d, J=8.7 Hz, 1H), 7.59-7.51 (m, 1H), 7.29 (d, J=2.1 Hz, 1H), 7.24 (s, 1H), 6.93-6.79 (m, 2H), 6.11 (d, J=0.8 Hz, 1H), 4.32-4.24 (m, 1H), 3.82 (s, 3H), 3.71 (s, 3H), 3.58-3.48 (m, 1H), 2.42 (s, 3H), 2.01-1.93 (m, 2H), 1.83-1.72 (m, 2H), 1.49-1.36 (m, 2H), 1.36-1.21 (m, 2H).

### Example 247: 2-ethyl-6-[[5-[5-(2-hydroxy-2-methyl-propoxy)-2-methyl-4-pyridyl]pyrazolo[1,5-a]pyridin-2-yl]amino]-4-methyl-pyridazin-3-one

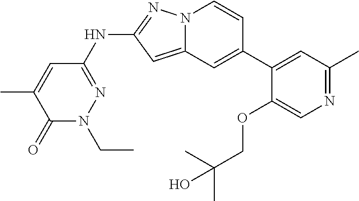

Step A: 6-chloro-2-ethyl-4-methylpyridazin-3(2H)-one. To a mixture of 6-chloro-4-methylpyridazin-3(2H)-one (1.0 g, 6.9 mmol), Cs₂CO₃ (5.64 g, 17.3 mmol) in DMF (13.8 mL) was added iodoethane (1.12 mL, 13.8 mmol). The mixture was stirred at 25° C. for 2 hr. The reaction was extracted with EtOAc/water/brine. The organics were then dried with Na₂SO₄, concentrated, and purified over silica gel: (24 g Gold ISCO column, 20-100% EtOAc/hexanes, 20 min gradient) providing the title compound (971 mg, 81.3%).

Step B: 2-ethyl-6-[[5-[5-(2-hydroxy-2-methyl-propoxy)-2-methyl-4-pyridyl]pyrazolo[1,5-a]pyridin-2-yl]amino]-4-methyl-pyridazin-3-one. A mixture of 1-((4-(2-aminopyrazolo[1,5-a]pyridin-5-yl)-6-methylpyridin-3-yl)oxy)-2-methylpropan-2-ol (102 mg, 0.327 mmol), 6-chloro-2-ethyl-4-methylpyridazin-3(2H)-one (62.0 mg, 0.359 mmol), t-BuBrettPhos Pd G3 (14.0 mg, 0.016 mmol), and Cs₂CO₃ (319 mg, 0.980 mmol) was placed in a Biotage vial: The capped vial was then evacuated and back-filled with N₂. The vial was evacuated again, and then an argon balloon was inserted and the solvent dioxane (0.6 mL) was added. The mixture was heated at 90° C. for 16 hr. The mixture was cooled down, solids were filtered off and rinsed with DMSO. The filtrate was purified over Basic ISCO (Gemini 5 uM NX-C18 110 angstrom LC column 150×21.2 mm; 10-100% AcN/20 mM aq NH₃ over 15 min; 40 mL/min) and lyophilized providing the title compound (69 mg, 47%). MS (ESI): mass calcd. for C₂₄H₂₈N₆O₃, 448.5. m/z found, 449.2 [M+H]⁺. ¹H NMR (500 MHz, Methanol-d4) δ 8.41-8.34 (m, 1H), 8.22 (s, 1H), 7.81-7.76 (m, 1H), 7.37 (s, 1H), 7.28-7.22 (m, 1H), 7.06 (dd, J=7.2, 2.0 Hz, 1H), 6.90-6.82 (m, 1H), 4.19 (q, J=7.2 Hz, 2H), 3.92 (s, 2H), 2.53 (s, 3H), 2.19 (d, J=1.3 Hz, 3H), 1.41 (t, J=7.2 Hz, 3H), 1.26 (s, 6H).

### Example 248: N-[5-[2-methyl-5-[[(1R,4R)-5-oxa-2-azabicyclo[2.2.1]heptan-4-yl]methoxy]-4-pyridyl]pyrazolo[1,5-a]pyridin-2-yl]cyclopropanecarboxamide

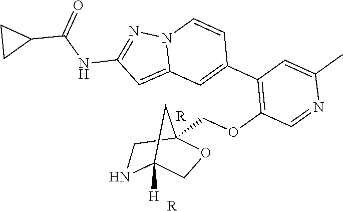

A mixture of tert-butyl (1R,4R)-1-(hydroxymethyl)-2-oxa-5-azabicyclo[2.2.1]heptane-5-carboxylate (325 mg, 1.418 mmol), DMA (2.5 mL) and NaH (60% dispersion in mineral oil) (257 mg, 6.44 mmol) was stirred at 40-50° C. for 5 min. Then N-(5-(5-fluoro-2-methylpyridin-4-yl)pyrazolo[1,5-a]pyridin-2-yl)cyclopropanecarboxamide (Intermediate 6, 200 mg, 0.644 mmol) was added. The reaction mixture was heated at 50° C. for 4 h, The reaction was cooled to RT, and worked-up with EtOAc/water/brine. The combined organic layers were dried (Na₂SO₄) and concentrated. The residue was purified by silica gel (12 g Gold ISCO column, 20-100% EtOAc/hexanes) providing the Boc-protected compound. To the Boc-protected compound in DCM (2.5 mL), CF₃COOH (0.5 mL) was added. The reaction mixture was heated at 50° C. for 10 min, The reaction was cooled to RT, and worked-up with saturated NaHCO₃ and 10% MeOH/DCM. The combined organic layers were dried (Na₂SO₄), concentrated and purified by silica gel (4 g Gold ISCO column, 0-20% MeOH (with 2M NH₃)/DCM, 15 min gradient) providing (126 mg, 46.7%). MS (ESI): mass calcd. for C₂₃H₂₅N₅O₃, 419.5. m/z found, 420.2 [M+H]⁺. ¹H NMR (500 MHz, Methanol-d4) δ 8.44-8.39 (m, 1H), 8.28 (s, 1H), 7.84-7.81 (m, 1H), 7.41-7.37 (m, 1H), 7.07 (dd, J=7.3, 2.0 Hz, 1H), 6.90 (s, 1H), 4.49 (s, 2H), 3.93-3.87 (m, 2H), 3.80 (s, 1H), 3.33-3.31 (m, 1H), 3.08-3.00 (m, 1H), 2.53 (s, 3H), 1.98-1.71 (m, 3H), 1.02-0.98 (m, 2H), 0.93-0.87 (m, 2H).

### Example 249: N-[5-[2-methyl-5-[[(1R,4R)-2-methyl-5-oxa-2-azabicyclo[2.2.1]heptan-4-yl]methoxy]-4-pyridyl]pyrazolo[1,5-a]pyridin-2-yl]cyclopropanecarboxamide

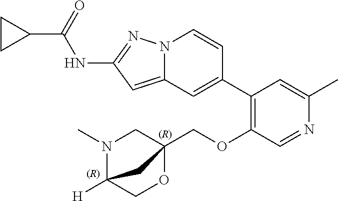

A mixture of N-[5-[2-methyl-5-[[(1R,4R)-5-oxa-2-azabicyclo[2.2.1]heptan-4-yl]methoxy]-4-pyridyl]pyrazolo[1,5-a]pyridin-2-yl]cyclopropanecarboxamide (Example 249, 108 mg, 0.257 mmol), paraformaldehyde (34.8 mg, 0.386 mmol) in MeOH (5 mL) was stirred for 15 min. Then NaCNBH₄ was added. The reaction mixture was stirred at RT at for 2 h, The reaction was worked-up with EtOAc/water. The combined organic layers were dried (Na₂SO₄) and concentrated. The residue was purified by silica gel (12 g Gold ISCO column, 0-20% MeOH (with 2M NH₃) in DCM, 20 min gradient) providing the title compound (76 mg, 68%). MS (ESI): mass calcd. for C₂₄H₂₇N₅O₃, 433.5. m/z found, 434.1 [M+H]⁺. ¹H NMR (500 MHz, Methanol-d4) δ 8.45-8.37 (m, 1H), 8.25 (s, 1H), 7.86-7.78 (m, 1H), 7.37 (s, 1H), 7.06 (dd, J=7.3, 2.0 Hz, 1H), 6.90 (s, 1H), 4.45-4.37 (m, 2H), 4.15-4.07 (m, 1H), 3.77 (dd, J=8.2, 1.9 Hz, 1H), 3.53-3.47 (m, 1H), 2.91 (d, J=10.2 Hz, 1H), 2.70 (dd, J=10.2, 1.6 Hz, 1H), 2.52 (s, 3H), 2.43 (s, 3H), 2.01-1.94 (m, 1H), 1.90-1.84 (m, 1H), 1.84-1.79 (m, 1H), 1.03-0.97 (m, 2H), 0.92-0.86 (m, 2H).

### Example 250: (1R,2R)-2-methyl-N-[5-[2-methyl-5-[[(1R,4R)-2-methyl-5-oxa-2-azabicyclo[2.2.1]heptan-4-yl]methoxy]-4-pyridyl]pyrazolo[1,5-a]pyridin-2-yl]cyclopropanecarboxamide

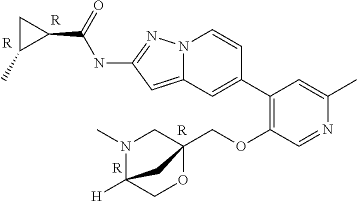

A mixture of (1R,2R)—N-(5-(5-(((1R,4R)-2-oxa-5-azabicyclo[2.2.1]heptan-1-yl)methoxy)-2-methylpyridin-4-yl)pyrazolo[1,5-a]pyridin-2-yl)-2-methylcyclopropane-1-carboxamide (Example 234; 73 mg, 0.168 mmol), and paraformaldehyde (22.7 mg, 0.253 mmol) in MeOH (3.3 mL) was stirred at 40° C. for 5 min. Then NaCNBH₄ (31.7 mg, 0.505 mmol) was added. The reaction mixture was stirred at 40° C. for 1 h, The reaction was worked-up with EtOAc/water/brine. The combined organic layers were dried (Na₂SO₄) and concentrated. The residue was purified by silica gel (4 g Gold ISCO column, 0-10% 2M NH₃ in MeOH/DCM, 30 min gradient) providing the title compound (55 mg, 73%). MS (ESI): mass calcd. for C₂₅H₂₉N₅O₃, 447.5. m/z found, 448.3 [M+H]⁺. ¹H NMR (500 MHz, Methanol-d4) δ 8.45-8.39 (m, 1H), 8.26 (s, 1H), 7.86-7.79 (m, 1H), 7.39 (s, 1H), 7.08 (dd, J=7.3, 2.0 Hz, 1H), 6.89 (s, 1H), 4.45-4.37 (m, 2H), 4.13-4.10 (m, 1H), 3.76 (dd, J=8.1, 1.9 Hz, 1H), 3.46 (s, 1H), 2.89 (d, J=10.1 Hz, 1H), 2.65 (dd, J=10.1, 1.5 Hz, 1H), 2.53 (s, 3H), 2.41 (s, 3H), 1.98-1.92 (m, 1H), 1.83-1.77 (m, 1H), 1.64-1.56 (m, 1H), 1.44-1.35 (m, 1H), 1.22-1.18 (m, 1H), 1.17 (d, J=6.0 Hz, 3H), 0.76-0.71 (m, 1H).

### Example 251: 5-[2-methyl-5-[[(1S,4S)-5-oxa-2-azabicyclo[2.2.1]heptan-4-yl]methoxy]-4-pyridyl]-N-(5-methylpyrazin-2-yl)pyrazolo[1,5-a]pyridin-2-amine

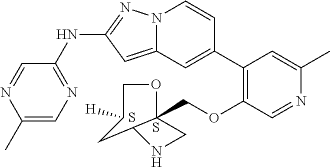

Step A: 5-(5-fluoro-2-methylpyridin-4-yl)-N-(5-methylpyrazin-2-yl)pyrazolo[1,5-a]pyridin-2-amine. A mixture of 5-(5-fluoro-2-methylpyridin-4-yl)pyrazolo[1,5-a]pyridin-2-amine (1.00 g, 4.13 mmol), 2-bromo-5-methylpyrazine (785 mg, 4.54 mmol), t-BuBrettPhos Pd G3 (176 mg, 0.206 mmol) and Cs₂CO₃ (4.03 g, 12.3 mmol) was placed in a vial: The capped vial was then evacuated and back-filled with N₂. The vial was evacuated again, and then an argon balloon was inserted and the solvent dioxane (0.6 mL) was added. The mixture was heated at 90° C. for 16 hr. The mixture was cooled down, solids were filtered off, and then worked-up with EtOAc/water/brine. The crude product was purified over silica gel (silica gel: 40 g Gold ISCO column, 20-100% EtOAc/hexanes, 15 min gradient.) providing the title compound (1.38 g, 57.2%). MS (ESI): mass calcd. for C₁₈H₁₅FN₆, 334.1. m/z found, 334.2 [M+H]⁺.

Step B: 5-[2-methyl-5-[[(1S,4S)-5-oxa-2-azabicyclo[2.2.1]heptan-4-yl]methoxy]-4-pyridyl]-N-(5-methylpyrazin-2-yl)pyrazolo[1,5-a]pyridin-2-amine was prepared in the similar manner of Example 238 where 5-(5-fluoro-2-methylpyridin-4-yl)-N-(5-methylpyrazin-2-yl)pyrazolo[1,5-a]pyridin-2-amine and tert-butyl (1S,4S)-1-(hydroxymethyl)-2-oxa-5-azabicyclo[2.2.1]heptane-5-carboxylate were used instead of N-(2,6-dimethylpyrimidin-4-yl)-5-(5-fluoro-2-methylpyridin-4-yl)pyrazolo[1,5-a]pyridin-2-amine and tert-butyl (1R,4R)-1-(hydroxymethyl)-2-oxa-5-azabicyclo[2.2.1]heptane-5-carboxylate. MS (ESI): mass calcd. for C₂₄H₂₅N₇O₂, 443.5. m/z found, 444.2 [M+H]⁺. ¹H NMR (500 MHz, Methanol-d4) δ 8.56 (d, J=1.5 Hz, 1H), 8.45-8.40 (m, 1H), 8.28 (s, 1H), 8.15-8.11 (m, 1H), 7.79-7.74 (m, 1H), 7.39 (s, 1H), 7.03 (dd, J=7.2, 2.0 Hz, 1H), 6.81-6.77 (m, 1H), 4.48 (s, 2H), 3.91 (dd, J=7.6, 1.8 Hz, 1H), 3.86 (d, J=7.5, 0.9 Hz, 1H), 3.70-3.66 (m, 1H), 3.03-2.95 (m, 2H), 2.54 (s, 3H), 2.44 (s, 3H), 1.89-1.84 (m, 1H), 1.81-1.76 (m, 1H).

### Example 252: N-(2,6-dimethylpyrimidin-4-yl)-5-[5-[[(1R,4R)-5-oxa-2-azabicyclo[2.2.1]heptan-4-yl]methoxy]-2-(trifluoromethyl)-4-pyridyl]pyrazolo[1,5-a]pyridin-2-amine

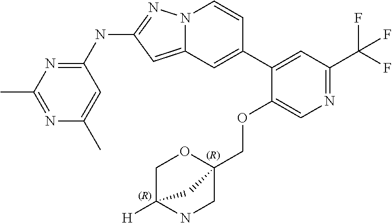

Step A: 5-(5-chloro-2-(trifluoromethyl)pyridin-4-yl)pyrazolo[1,5-a]pyridin-2-amine. To the mixture of 5-chloro-4-iodo-2-(trifluoromethyl)pyridine (1.00 g, 3.25 mmol), tert-butyl (5-(4,4,5,5-tetramethyl-1,3,2-dioxaborolan-2-yl)pyrazolo[1,5-a]pyridin-2-yl)carbamate (1.32 g, 3.70 mmol) and Bis(diphenylphosphino)ferrocene]dichloropalladium(II) Dichloromethane (68.9 mg, 0.0942 mmol) in dioxane (10 mL), saturated NaHCO₃ (3 mL) was added. The mixture was heated at 100° C. for 16 h. The mixture was cooled down, and concentrated. H₂O (20 mL) was added and stirred for 8 h. The solids were collected and dried, and then MeOH (10 mL), and HCl (2 mL, 4M in dioxane) were added. The mixture was stirred at RT for 8 h. After concentration the title compound was obtained (650 mg, 63.9%). MS (ESI): mass calcd. for C₁₃H₈ClF₃N₄, 511.5. m/z found, 512.2 [M+H]⁺.

Step B: Tert-butyl (1R,4R)-1-(((4-(2-aminopyrazolo[1,5-a]pyridin-5-yl)-6-(trifluoromethyl)pyridin-3-yl)oxy)methyl)-2-oxa-5-azabicyclo[2.2.1]heptane-5-carboxylate. A mixture of (1R,4R)-1-(hydroxymethyl)-2-oxa-5-azabicyclo[2.2.1]heptane-5-carboxylate (143 mg, 0.624 mmol) and NaH (60% dispersion in mineral oil; 191 mg, 4.80 mmol) in DMA (1 mL) was stirred at RT for 10 min, after which 5-(5-chloro-2-(trifluoromethyl)pyridin-4-yl)pyrazolo[1,5-a]pyridin-2-amine (150 mg, 0.48 mmol) was added. The mixture was then stirred at room temperature for 30 min. Saturated NH₄Cl (1 mL) was added carefully. The mixture was extracted with DCM (2×20 mL). The combined organics were concentrated and the resulting product was purified over silica gel using acetone/DCM providing the title compound (80 mg, 33%). MS (ESI): mass calcd. for C₂₄H₂₆F₃N₅O₄, 505.19. m/z found, 506.1 [M+H]⁺.

Step C: N-(2,6-dimethylpyrimidin-4-yl)-5-[5-[[(1R,4R)-5-oxa-2-azabicyclo[2.2.1]heptan-4-yl]methoxy]-2-(trifluoromethyl)-4-pyridyl]pyrazolo[1,5-a]pyridin-2-amine. A mixture of 4-chloro-2,6-dimethylpyrimidine (19 mg, 0.13 mmol), tert-butyl (1RS,4R)-1-(((4-(2-aminopyrazolo[1,5-a]pyridin-5-yl)-6-(trifluoromethyl)pyridin-3-yl)oxy)methyl)-2-oxa-5-azabicyclo[2.2.1]heptane-5-carboxylate (70 mg, 0.13 mmol), BrettPhos Pd G3 (5.1 mg, 0.0056 mmol), and Cs₂CO₃ (174 mg, 0.535 mmol) was placed in a Biotage vial: The capped vial was then evacuated and back-filled with N₂. The vial was evacuated again, and then an argon balloon was inserted and the solvent dioxane (4 mL) was added. The mixture was heated at 150° C. for 1 hr. The mixture was cooled down, and solids were filtered off. The crude product was diluted with brine and EtOAc. The organic layer was separated. and concentrated under vacuo. The residue was flashed with 10% acetone/CH₂Cl₂ providing the Boc-protected compound. To the Boc-protected compound in CH₂CL₂/MeOH (1 mL/1 mL), TFA (0.5 mL) was added. The mixture was stirred at RT for 4 h. The mixture was concentrated and purified with FCC (10% (2M NH₃ MeOH/DCM) providing the title compound. MS (ESI): mass calcd. for C₂₅H₂₄F₃N₇O₂, 511.5. m/z found, 512.2 [M+H]⁺. ¹H NMR (500 MHz, Methanol-d4) δ 8.57 (s, 1H), 8.44 (dd, J=7.3, 1.0 Hz, 1H), 7.89-7.81 (m, 2H), 7.09-6.95 (m, 2H), 6.86 (s, 1H), 4.65 (s, 2H), 3.97-3.83 (m, 2H), 3.73-3.66 (m, 1H), 3.08-2.90 (m, 2H), 2.52 (s, 3H), 2.36 (s, 3H), 1.94-1.72 (m, 2H).

### Example 253: N-[5-[2-methyl-5-[[(1S,4S)-5-oxa-2-azabicyclo[2.2.1]heptan-4-yl]methoxy]-4-pyridyl]pyrazolo[1,5-a]pyridin-2-yl]cyclopropanecarboxamide

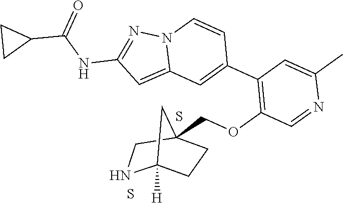

Step A: Tert-butyl (1S,4S)-1-(((methylsulfonyl)oxy)methyl)-2-oxa-5-azabicyclo[2.2.1]heptane-5-carboxylate. To a mixture of tert-butyl (1S,4S)-1-(hydroxymethyl)-2-oxa-5-azabicyclo[2.2.1]heptane-5-carboxylate (1.00 g, 4.36 mmol) and DMAP (1.33 g, 10.9 mmol) in DCM (4 mL) at 0° C., methanesulfonyl chloride (0.678 mL, 8.72 mmol) was added. The mixture was stirred at RT for 16 h. The mixture was worked up with EtOAc/water/brine and purified by silica gel (40 g Gold ISCO column: 25-100% EtOAc/hexanes) providing the title compound (1.25 g, 93.3%).

Step B. (1S,4S)-1-(((4-bromo-6-methylpyridin-3-yl)oxy)methyl)-2-oxa-5-azabicyclo[2.2.1]heptane-5-carboxylate. A mixture of tert-butyl (1S,4S)-1-(((methylsulfonyl)oxy)methyl)-2-oxa-5-azabicyclo[2.2.1]heptane-5-carboxylate (650 mg, 2.11 mmol), 4-bromo-6-methylpyridin-3-ol (795 mg, 4.23 mmol), and Cs₂CO₃ (2.07 g, 6.34 mmol) was stirred at 50° C. for 2 hr. The mixture was worked up with EtOAc/water/brine and purified by silica gel (40 g Gold ISCO column: 25-100% EtOAc/hexanes) providing the title compound (882 mg, 97.3%). MS (ESI): mass calcd. For C₁₇H₂₃BrN₂O₄, 398.08. m/z found, 399.1 [M+H]⁺.

Step C. N-[5-[2-methyl-5-[[(1S,4S)-5-oxa-2-azabicyclo[2.2.1]heptan-4-yl]methoxy]-4-pyridyl]pyrazolo[1,5-a]283yridine-2-yl]cyclopropanecarboxamide. A mixture of (1S,4S)-1-(((4-bromo-6-methylpyridin-3-yl)oxy)methyl)-2-oxa-5-azabicyclo[2.2.1]heptane-5-carboxylate (154 mg, 0.386 mmol), N-(5-(4,4,5,5-tetramethyl-1,3,2-dioxaborolan-2-yl)pyrazolo[1,5-a]pyridin-2-yl)cyclopropanecarboxamide (Intermediate 3; 157 mg, 0.482 mmol) and 0.5 M K₃PO₄ (0.771 mL, 0.386 mmol) in dioxane (1.5 mL) was placed in a vial. The capped vial was then evacuated and back-filled with N₂. The vial was evacuated again, then backfilled with N₂, and then 6-bromo-1H-pyrrolo[2,3-b]pyridine (157 mg, 0.482 mmol) and [1,1′-Bis(diphenylphosphino)ferrocene]dichloropalladium(II) Dichloromethane (31 mg, 0.038 mmol) were added. The mixture was heated at 90° C. for 1 hr. The mixture was worked up with EtOAc/water/brine and purified by silica gel (20-100% EtOAc/hexanes over 20 min) providing the Boc-protected compound. To the Boc-protected compound (155 mg, 0.298 mmol) in DCM (1.5 mL), CF₃COOH (0.3 mL) was added. The reaction mixture was stirred at RT for 16 h, The reaction was cooled to RT, and worked-up with saturated NaHCO₃ and 10% MeOH/DCM. The combined organic layers were dried (Na₂SO₄), concentrated and purified by silica gel (4 g Gold ISCO column, 0-10% MeOH (with 2M NH₃)/DCM, 20 min gradient) providing the title compound (94 mg, 75%). MS (ESI): mass calcd. for C₂₃H₂₅N₅O₃, 419.5. m/z found, 420.2 [M+H]⁺. ¹H NMR (500 MHz, Methanol-d4) δ 8.44-8.39 (m, 1H), 8.28 (s, 1H), 7.85-7.80 (m, 1H), 7.39 (s, 1H), 7.08 (dd, J=7.3, 2.0 Hz, 1H), 6.90 (s, 1H), 4.48 (s, 2H), 3.93-3.85 (m, 2H), 3.77 (s, 1H), 3.07-2.98 (m, 2H), 2.53 (s, 3H), 1.93-1.85 (m, 2H), 1.83-1.77 (m, 1H), 1.02-0.96 (m, 2H), 0.94-0.87 (m, 2H).

### Example 254: 5-[2-methyl-5-[[(1R,4R)-5-oxa-2-azabicyclo[2.2.1]heptan-4-yl]methoxy]-4-pyridyl]-N-(6-methylpyridazin-3-yl)pyrazolo[1,5-a]pyridin-2-amine

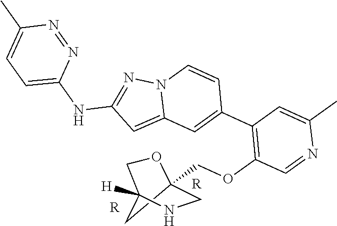

The title compound was prepared in the similar manner of Example 251 except 3-chloro-6-methylpyridazine was used instead of 2-bromo-5-methylpyrazine in step A; and tert-butyl (1R,4R)-1-(hydroxymethyl)-2-oxa-5-azabicyclo[2.2.1]heptane-5-carboxylate was used instead of tert-butyl (1S,4S)-1-(hydroxymethyl)-2-oxa-5-azabicyclo[2.2.1]heptane-5-carboxylate in Step B (39.5 mg, 58%). MS (ESI): mass calcd. for C₂₄H₂₅N₇O₂, 443.5. m/z found, 444.2 [M+H]⁺. ¹H NMR (500 MHz, Methanol-d4) δ 8.44-8.40 (m, 1H), 8.28 (s, 1H), 7.83-7.77 (m, 1H), 7.68 (d, J=9.2 Hz, 1H), 7.43 (d, J=9.2 Hz, 1H), 7.40 (s, 1H), 7.04 (dd, J=7.2, 1.9 Hz, 1H), 6.86 (s, 1H), 4.48 (s, 2H), 3.94-3.83 (m, 2H), 3.69 (s, 1H), 3.03-2.95 (m, 2H), 2.55 (d, J=14.2 Hz, 6H), 1.91-1.74 (m, 2H).

### Example 255: N-(6-cyclopropylpyridazin-3-yl)-5-[2-methyl-5-[[(1R,4R)-5-oxa-2-azabicyclo[2.2.1]heptan-4-yl]methoxy]-4-pyridyl]pyrazolo[1,5-a]pyridin-2-amine

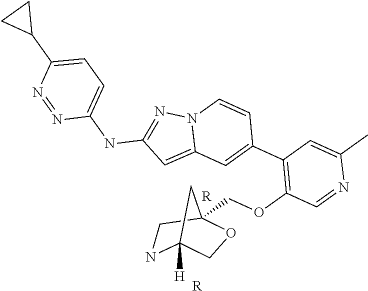

The title compound was prepared in the similar manner of Example 251 except 3-chloro-6-cyclopropylpyridazine was used instead of 2-bromo-5-methylpyrazine in step A; and tert-butyl (1R,4R)-1-(hydroxymethyl)-2-oxa-5-azabicyclo[2.2.1]heptane-5-carboxylate was used instead of tert-butyl (1S,4S)-1-(hydroxymethyl)-2-oxa-5-azabicyclo[2.2.1]heptane-5-carboxylate in step B (45.5 mg, 48%). MS (ESI): mass calcd. for C₂₆H₂₇N₇O₂, 469.5. m/z found, 470.2 [M+H]⁺. ¹H NMR (500 MHz, Methanol-d4) δ 8.45-8.38 (m, 1H), 8.28 (s, 1H), 7.85-7.77 (m, 1H), 7.64 (d, J=9.3 Hz, 1H), 7.40 (s, 1H), 7.29 (d, J=9.3 Hz, 1H), 7.03 (dd, J=7.2, 2.0 Hz, 1H), 6.85 (s, 1H), 4.48 (s, 2H), 3.95-3.84 (m, 2H), 3.69 (s, 1H), 3.05-2.95 (m, 2H), 2.54 (s, 3H), 2.23-2.13 (m, 1H), 1.91-1.84 (m, 1H), 1.81-1.76 (m, 1H), 1.11-1.05 (m, 2H), 1.01-0.94 (m, 2H).

### Example 256: 5-[2-methyl-5-[[(1R,4R)-2-methyl-5-oxa-2-azabicyclo[2.2.1]heptan-4-yl]methoxy]-4-pyridyl]-N-(6-methylpyridazin-3-yl)pyrazolo[1,5-a]pyridin-2-amine

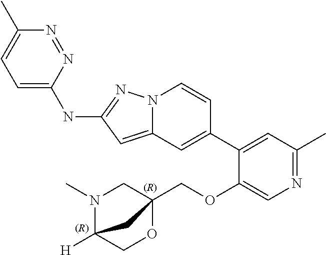

The title compound (10 mg, 32%) was prepared in the similar manner of Example 249, except 5-[2-methyl-5-[[(1R,4R)-5-oxa-2-azabicyclo[2.2.1]heptan-4-yl]methoxy]-4-pyridyl]-N-(6-methylpyridazin-3-yl)pyrazolo[1,5-a]pyridin-2-amine (Example 254) was used instead of N-[5-[2-methyl-5-[[(1R,4R)-5-oxa-2-azabicyclo[2.2.1]heptan-4-yl]methoxy]-4-pyridyl]pyrazolo[1,5-a]pyridin-2-yl]cyclopropanecarboxamide. MS (ESI): mass calcd. for C₂₅H₂₇N₇O₂, 457.5. m/z found, 458.2 [M+H]⁺. ¹H NMR (500 MHz, Methanol-d4) δ 8.45-8.41 (m, 1H), 8.26 (s, 1H), 7.82-7.78 (m, 1H), 7.68 (d, J=9.1 Hz, 1H), 7.43 (d, J=9.2 Hz, 1H), 7.40 (s, 1H), 7.03 (dd, J=7.2, 2.0 Hz, 1H), 6.87 (s, 1H), 4.42 (d, J=2.2 Hz, 2H), 4.12 (dd, J=8.1, 1.1 Hz, 1H), 3.78 (dd, J=8.2, 1.9 Hz, 1H), 3.49 (s, 1H), 2.92 (d, J=10.2 Hz, 1H), 2.72-2.67 (m, 1H), 2.56 (s, 3H), 2.53 (s, 3H), 2.42 (s, 3H), 2.00-1.95 (m, 1H), 1.85-1.80 (m, 1H).

### Example 257: 5-[2-methyl-5-[[(1R,4R)-5-oxa-2-azabicyclo[2.2.1]heptan-4-yl]methoxy]-4-pyridyl]-N-(5-methylpyrazin-2-yl)pyrazolo[1,5-a]pyridin-2-amine

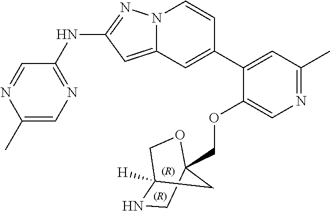

The title compound (8.4 mg, 10%) was prepared in the similar manner of Example 251 except tert-butyl (1R,4R)-1-(hydroxymethyl)-2-oxa-5-azabicyclo[2.2.1]heptane-5-carboxylate was used instead of tert-butyl (1S,4S)-1-(hydroxymethyl)-2-oxa-5-azabicyclo[2.2.1]heptane-5-carboxylate in step B. MS (ESI): mass calcd. for C₂₄H₂₅N₇O₂, 443.5. m/z found, 444.2 [M+H]⁺. ¹H NMR (600 MHz, Methanol-d4) δ 8.55 (d, J=1.5 Hz, 1H), 8.46-8.39 (m, 1H), 8.28 (s, 1H), 8.15-8.10 (m, 1H), 7.79-7.75 (m, 1H), 7.39 (s, 1H), 7.02 (dd, J=7.2, 1.9 Hz, 1H), 6.79 (d, J=0.9 Hz, 1H), 4.48 (s, 2H), 3.91 (dd, J=7.6, 1.8 Hz, 1H), 3.86 (d, J=7.6, 0.9 Hz, 1H), 3.70-3.66 (m, 1H), 3.03-2.96 (m, 2H), 2.53 (s, 3H), 2.44 (s, 3H), 1.89-1.84 (m, 1H), 1.80-1.76 (m, 1H).

### Example 258: (*R)—N-(2,6-dimethylpyrimidin-4-yl)-5-[2-methyl-5-(8-oxaspiro[4.4]nonan-7-ylmethoxy)-4-pyridyl]pyrazolo[1,5-a]pyridin-2-amine

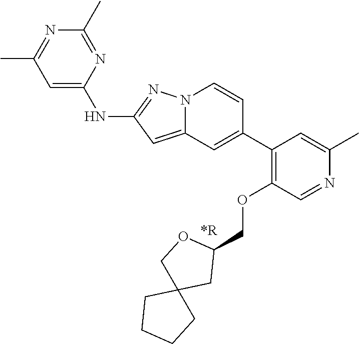

The title compound (75.5 mg, 42%) was prepared as the first eluting peak after purification by SFC-11 over DAICEL CHIRALPAK AD 250 mm×30 mm×10 μm (eluent: 45% to 45% (v/v) EtOH with 0.1% NH₃·H₂O in supercritical CO₂) as described in Example 219. MS (ESI): mass calcd. for C₂₈H₃₂N₆O₂, 484.6. m/z found, 485.1 [M+H]⁺. ¹H NMR (400 MHz, CDCl₃) δ 8.36 (d, J=7.2 Hz, 1H), 8.29 (s, 1H), 7.68 (d, J=0.8 Hz, 1H), 7.42 (s, 1H), 7.18 (s, 1H), 7.07 (s, 1H), 7.00 (dd, J=2.0, 7.2 Hz, 1H), 6.57 (s, 1H), 4.36-4.29 (m, 1H), 4.12 (d, J=4.4 Hz, 2H), 3.62-3.57 (m, 2H), 2.60 (s, 3H), 2.56 (s, 3H), 2.45 (s, 3H), 1.93-1.84 (m, 1H), 1.76-1.72 (m, 1H), 1.66-1.49 (m, 8H) 1H NMR (400 MHz, DMSO-d6) δ 10.21 (s, 1H), 8.59 (d, J=7.2 Hz, 1H), 8.34 (s, 1H), 7.85 (s, 1H), 7.36 (s, 1H), 7.03 (dd, J=1.6, 7.2 Hz, 1H), 6.98 (br. s., 1H), 6.89 (br. s., 1H), 4.27-4.20 (m, 1H), 4.19-4.09 (m, 2H), 3.49-3.45 (m, 2H), 2.47 (s, 3H), 2.46 (s, 3H), 2.30 (s, 3H), 1.90-1.82 (m, 1H), 1.75-1.66 (m, 1H), 1.59-1.42 (m, 8H).

### Example 259: 2-[[4-[2-[(2,6-dimethylpyrimidin-4-yl)amino]pyrazolo[1,5-a]pyridin-5-yl]-6-methyl-3-pyridyl]oxymethyl]-1,1,1,3,3,3-hexafluoro-propan-2-ol

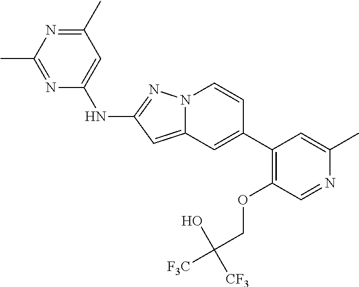

Step A: 2-(((4-Bromo-6-methylpyridin-3-yl)oxy)methyl)-1,1,1,3,3,3-hexafluoropropan-2-ol. A 30 mL microwave vial was charged with 4-bromo-6-methylpyridin-3-ol (1.00 g, 5.32 mmol) and DMF (10.6 mL). At RT, cesium carbonate was added (3.47 g, 10.6 mmol) followed by 2,2-bis(trifluoromethyl)oxirane (1.92 g, 10.6 mmol). The reaction mixture was heated at 100° C. overnight. The reaction mixture was then cooled and dissolved in EtOAc, and then washed with water and brine. The organic layers were dried with Na₂SO₄, and then filtered and concentrated to dryness under reduced pressure. The crude product was purified via flash column chromatography with silica gel (20-100% EtOAc/hexanes) to afford the desired product (1.50 g, 77%). MS (ESI): mass calcd. for C₁₀H₈BrF₆NO₂, 367.0. m/z found, 368.0 [M+H]⁺. 1H NMR (500 MHz, Chloroform-d) δ 8.06 (s, 1H), 7.42 (s, 1H), 4.45 (s, 2H), 2.97-2.88 (m, 1H), 2.50 (s, 3H).

Step B: 2-4(4-(2-Aminopyrazolo[1,5-a]pyridin-5-yl)-6-methylpyridin-3-yl)oxy)methyl)-1,1,1,3,3,3-hexafluoropropan-2-ol. A30 mL microwave vial containing a stir bar was charged with 2-(((4-bromo-6-methylpyridin-3-yl)oxy)methyl)-1,1,1,3,3,3-hexafluoropropan-2-ol (1.50 g, 4.08 mmol), Intermediate 14 (1.37 g, 5.30 mmol), 1,1′-bis(diphenylphosphino)ferrocene-palladium(ii)dichloride dichloromethane complex (337 mg, 0.41 mmol), aqueous K₃PO₄ (0.5M, 8 mL) and 1,4-dioxane (16 mL). The reaction mixture was degassed under N₂ for 10 min. The vial was placed into a pre-heated plate at 90° C. for 1 h. The reaction mixture was then cooled and dissolved in EtOAc, washed with water and brine, and the organic layers were dried with Na₂SO₄ and then filtered and concentrated to dryness under reduced pressure. The crude product was purified via flash column chromatography purified over silica gel (20-100% EtOAc with 10% MeOH/hexanes) to afford desired product (1.3 g, 76%). MS (ESI): mass calcd. for C₁₄H₁₇F₆N₄O₂, 420.1. m/z found, 421.1 [M+H]⁺. 1H NMR (500 MHz, Chloroform-d) δ 8.21-8.06 (m, 2H), 7.39 (dd, J=1.9, 0.9 Hz, 1H), 7.15 (s, 1H), 6.68 (dd, J=7.2, 2.0 Hz, 1H), 5.77 (d, J=0.8 Hz, 1H), 4.42 (s, 2H), 4.01 (s, 2H), 3.50 (s, 1H), 2.50 (s, 3H).

Step C: 2-(((4-(2-((2,6-Dimethylpyrimidin-4-yl)amino)pyrazolo[1,5-a]pyridin-5-yl)-6-methylpyridin-3-yl)oxy)methyl)-1,1,1,3,3,3-hexafluoropropan-2-ol. A 30 mL microwave vial containing a stir bar was charged with 2-4(4-(2-aminopyrazolo[1,5-a]pyridin-5-yl)-6-methylpyridin-3-yl)oxy)methyl)-1,1,1,3,3,3-hexafluoropropan-2-ol (250 mg, 0.60 mmol), 4-chloro-2,6-dimethylpyrimidine (93.3 mg, 0.65 mmol), tBuBrettPhos Pd G3 (25.4 mg, 0.03 mmol), cesium carbonate (0.58 g, 1.78 mmol), and 1,4-dioxane (11 mL). The reaction mixture was degassed under vacuum with sonication for 2 minutes, then vented to argon. The vial was placed into a pre-heated plate at 80° C. for 16 h. The reaction mixture was then filtered, concentrated to dryness under reduced pressure to afford the crude product which was purified by flash column chromatography (eluent: 0-10% 2M NH₃ in CH₃OH/DCM)) to afford the title compound (172 mg, 55%) as a white solid. MS (ESI): mass calcd. for C₂₃H₂₀F₆N₆O₂, 526.4. m/z found, 527.15 [M+H]⁺. 1H NMR (400 MHz, Chloroform-d) δ 8.31 (d, J=17.2 Hz, 2H), 7.72 (s, 1H), 7.51 (d, J=1.8 Hz, 1H), 7.17 (s, 1H), 6.87 (s, 1H), 6.81 (dd, J=7.2, 1.9 Hz, 1H), 6.46 (s, 1H), 4.54 (s, 2H), 3.51 (s, OH), 2.57 (s, 3H), 2.48 (s, 3H), 2.33 (s, 3H).

### Example 260: 1-[[4-[2-(imidazo[1,2-b]pyridazin-6-ylamino)pyrazolo[1,5-a]pyridin-5-yl]-6-methyl-3-pyridyl]oxy]-2-methyl-propan-2-ol

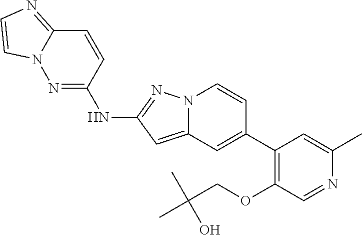 [SMILES: Cc1cc(-c2ccn3nc(Nc4ccc5nccn5n4)cc3c2)c(OCC(C)(C)O)cn1]

A 30 mL microwave vial containing a stir bar was charged with 1-((4-(2-aminopyrazolo[1,5-a]pyridin-5-yl)-6-methylpyridin-3-yl)oxy)-2-methylpropan-2-ol (250 mg, 0.80 mmol), 6-chloroimidazo[1,2-b]pyridazine (138 mg, 0.88 mmol), tBuBrettPhos Pd G3 (34 mg, 0.04 mmol), cesium carbonate (0.78 g, 2.40 mmol), and 1,4-dioxane (16 mL). The reaction mixture was degassed under vacuum with sonication for 2 minutes, then vented to argon. The vial was placed into a pre-heated plate at 80° C. for 16 h. The reaction mixture was then filtered, concentrated to dryness under reduced pressure to afford the crude product which was purified by flash column chromatography (eluent: 0-10% 2M NH₃ in CH₃OH/DCM)) to afford the title compound (89 mg, 26%) as a white solid. MS (ESI): mass calcd. for C₂₃H₂₃N₇O₂, 429.5. m/z found, 430.20 [M+H]⁺. 1H NMR (500 MHz, Chloroform-d) δ 8.30-8.22 (m, 2H), 8.20 (d, J=3.7 Hz, 1H), 7.81-7.71 (m, 2H), 7.63-7.57 (m, 2H), 7.15 (s, 1H), 6.98-6.89 (m, 2H), 6.79 (d, J=9.6 Hz, 1H), 3.97 (s, 2H), 3.51 (s, 1H), 2.56 (s, 3H), 1.35 (s, 6H).

### Example 261: 1-[[4-[2-[(2-tert-butyl-6-methyl-pyrimidin-4-yl)amino]pyrazolo[1,5-a]pyridin-5-yl]-6-methyl-3-pyridyl]oxy]-2-methyl-propan-2-ol

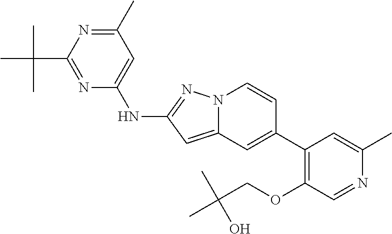 [SMILES: Cc1cc(-c2ccn3nc(Nc4cc(C)nc(C(C)(C)C)n4)cc3c2)c(OCC(C)(C)O)cn1]

A 30 mL microwave vial containing a stir bar was charged with 1-((4-(2-aminopyrazolo[1,5-a]pyridin-5-yl)-6-methylpyridin-3-yl)oxy)-2-methylpropan-2-ol (250 mg, 0.80 mmol), 2-(tert-butyl)-4-chloro-6-methylpyrimidine (163 mg, 0.88 mmol), tBuBrettPhos Pd G3 (34 mg, 0.04 mmol), cesium carbonate (0.78 g, 2.40 mmol), and 1,4-dioxane (16 mL). The reaction mixture was degassed under vacuum with sonication for 2 minutes, then vented to argon. The vial was placed into a pre-heated plate at 80° C. for 16 h. The reaction mixture was then filtered, concentrated to dryness under reduced pressure to afford the crude product which was purified by flash column chromatography (eluent: 0-10% 2M NH₃ in CH₃OH/DCM)) to afford the title compound (182 mg, 49%) as a white solid. MS (ESI): mass calcd. for C₂₆H₃₂N₆O₂, 460.6. m/z found, 461.20 [M+H]⁺. 1H NMR (500 MHz, Chloroform-d) δ 8.27 (s, 1H), 8.19 (dt, J=7.1, 0.9 Hz, 1H), 7.68 (s, 1H), 7.56 (dd, J=2.0, 0.9 Hz, 1H), 7.08 (s, 1H), 6.88 (s, 1H), 6.80 (dd, J=7.2, 1.9 Hz, 1H), 6.41 (s, 1H), 3.99 (s, 2H), 3.35 (s, 1H), 2.53 (s, 3H), 2.36 (s, 3H), 1.45 (s, 9H), 1.38 (s, 6H).

### Example 262: 3-[[4-[2-[(2,6-dimethylpyrimidin-4-yl)amino]pyrazolo[1,5-a]pyridin-5-yl]-6-methyl-3-pyridyl]oxy]-2,2-dimethyl-propanenitrile

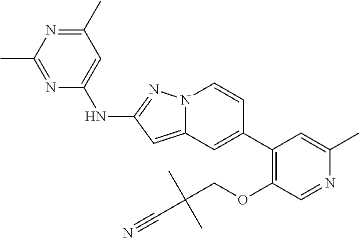

The title compound was prepared (212 mg, 69%) by the similar method of Example 1 using 3-hydroxy-2,2-dimethylpropanenitrile instead of (R)-(1-methylpyrrolidin-3-yl)methanol and Intermediate 15 instead of N-(5-(5-chloro-2-cyanopyridin-4-yl)pyrazolo[1,5-a]pyridin-2-yl)cyclopropanecarboxamide. MS (ESI): mass calcd. for C₂₄H₂₅N₇O, 427.5. m/z found, 428.2 [M+H]⁺. 1H NMR (500 MHz, Chloroform-d) δ8.59-8.50 (m, 2H), 8.22 (s, 1H), 7.69 (dd, J=2.0, 0.9 Hz, 1H), 7.22 (s, 1H), 7.07 (s, 1H), 7.01 (dd, J=7.2, 2.0 Hz, 1H), 6.62 (s, 1H), 3.99 (s, 2H), 2.59 (d, J=16.5 Hz, 6H), 2.43 (s, 3H), 1.40 (s, 6H).

### Example 263: (*S)-3-[[4-[2-[(2,6-dimethylpyrimidin-4-yl)amino]pyrazolo[1,5-a]pyridin-5-yl]-6-methyl-3-pyridyl]oxy]-1,1,1-trifluoro-2-methyl-propan-2-ol

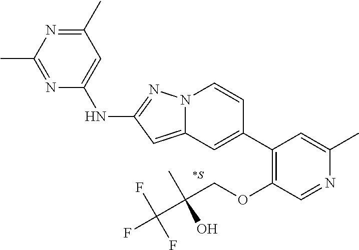

Step A: 3-((4-Bromo-6-methylpyridin-3-yl)oxy)-1,1,1-trifluoro-2-methylpropan-2-ol. A 30 mL microwave vial was charged with 4-bromo-6-methylpyridin-3-ol (1.00 g, 5.32 mmol) and DMF (10.6 mL). At RT, cesium carbonate was added (3.47 g, 10.6 mmol) followed by 2-methyl-2-(trifluoromethyl)oxirane (1.34 g, 10.6 mmol). The reaction mixture was heated at 100° C. overnight. The reaction mixture was then cooled and dissolved in EtOAc, and then washed with water and brine. The organic layers were dried with Na₂SO₄ then filtered and concentrated to dryness under reduced pressure. The crude product was purified via flash column chromatography purified over silica gel (20-100% EtOAc/hexanes) to afford desired product (700 mg, 42%). MS (ESI): mass calcd. for C₁₀H₁₁BrF₃NO₂, 313.0. m/z found, 314.0 [M+H]⁺. 1H NMR (500 MHz, Chloroform-d) δ 8.04 (s, 1H), 7.37 (bs, 1H), 4.76 (s, 1H), 4.24 (d, J=9.6 Hz, 1H), 4.09 (dd, J=9.6, 1.0 Hz, 1H), 2.48 (s, 3H), 1.56 (d, J=1.0 Hz, 3H).

Step B: 3-((4-(2-Aminopyrazolo[1,5-a]pyridin-5-yl)-6-methylpyridin-3-yl)oxy)-1,1,1-trifluoro-2-methylpropan-2-ol. A 30 mL microwave vial containing a stir bar was charged with 3-((4-bromo-6-methylpyridin-3-yl)oxy)-1,1,1-trifluoro-2-methylpropan-2-ol (700 mg, 2.23 mmol), Intermediate 14, 5-(4,4,5,5-tetramethyl-1,3,2-dioxaborolan-2-yl)pyrazolo[1,5-a]pyridin-2-amine (751 mg, 2.90 mmol), 1,1′-bis(diphenylphosphino)ferrocene-palladium(ii)dichloride dichloromethane complex (184 mg, 0.22 mmol), aqueous K₃PO₄ (0.5M, 4 mL) and 1,4-dioxane (9 mL). The reaction mixture was degassed under N₂ for 10 min. The vial was placed into a pre-heated plate at 90° C. for 1 h. The reaction mixture was cooled and dissolved in EtOAc, washed with water and brine, and the organic layers were dried with Na₂SO₄, filtered, and then concentrated to dryness under reduced pressure. The crude product was purified via flash column chromatography purified over silica gel (20-100% EtOAc with 10% MeOH/hexanes) to afford the desired product (500 mg, 61%). MS (ESI): mass calcd. for C₁₇H₁₇F₃N₄O₂, 366.1.0. m/z found, 367.1 [M+H]⁺. 1H NMR (500 MHz, Chloroform-d) δ 8.08-7.99 (m, 2H), 7.33 (dd, J=2.0, 0.9 Hz, 1H), 7.04 (s, 1H), 6.63 (dd, J=7.2, 2.0 Hz, 1H), 5.67 (d, J=0.8 Hz, 1H), 5.60 (s, 1H), 4.09 (d, J=9.6 Hz, 1H), 4.04-3.96 (m, 1H), 3.37 (s, 2H), 2.41 (s, 3H), 1.35 (s, 3H)

Step C: 3-((4-(2-((2,6-Dimethylpyrimidin-4-yl)amino)pyrazolo[1,5-a]pyridin-5-yl)-6-methylpyridin-3-yl)oxy)-1,1,1-trifluoro-2-methylpropan-2-ol. A 30 mL microwave vial containing a stir bar was charged with 3-((4-(2-aminopyrazolo[1,5-a]pyridin-5-yl)-6-methylpyridin-3-yl)oxy)-1,1,1-trifluoro-2-methylpropan-2-ol (500 mg, 1.37 mmol), 4-chloro-2,6-dimethylpyrimidine (214 mg, 1.50 mmol), tBuBrettPhos Pd G3 (58.3 mg, 0.07 mmol), cesium carbonate (1.33 g, 4.10 mmol), and 1,4-dioxane (10 mL). The reaction mixture was degassed under vacuum with sonication for 2 minutes, then vented with argon. The vial was placed into a pre-heated plate at 80° C. for 16 h. The reaction mixture was then filtered, and then concentrated to dryness under reduced pressure to afford the crude product which was purified by flash column chromatography (eluent: 0-10% 2M NH₃ in CH₃OH/DCM)) to afford the title compound (450 mg, 70%) as a white solid. MS (ESI): mass calcd. for C₂₃H₂₃F₃N₆O₂, 472.2. m/z found, 473.2 [M+H]⁺. 1H NMR (400 MHz, Chloroform-d) δ 8.52 (s, 1H), 8.40 (d, J=7.2 Hz, 1H), 8.23 (s, 1H), 7.57 (d, J=2.0 Hz, 1H), 7.14 (s, 1H), 6.88-6.78 (m, 2H), 6.52 (s, 1H), 5.88 (s, 1H), 4.28 (d, J=9.6 Hz, 1H), 4.14 (d, J=9.6 Hz, 1H), 2.52 (d, J=3.6 Hz, 6H), 2.30 (s, 3H), 1.51 (s, 3H).

Step D: (*S)-3-((4-(2-((2,6-dimethylpyrimidin-4-yl)amino)pyrazolo[1,5-a]pyridin-5-yl)-6-methylpyridin-3-yl)oxy)-1,1,1-trifluoro-2-methylpropan-2-ol. The mixture of 3-((4-(2-((2,6-dimethylpyrimidin-4-yl)amino)pyrazolo[1,5-a]pyridin-5-yl)-6-methylpyridin-3-yl)oxy)-1,1,1-trifluoro-2-methylpropan-2-ol was separated via chiral SFC (SFC-B, 2×25 cm, mobile phase: 30% MeOH, 70% CO₂), and then chiral SFC (OJ-H 2×25 cm, mobile phase: 30% 0.1% NH₃ in MeOH, 70% CO₂) to afford two peaks. The first elute isomer was designated as (*S) isomer (174 mg, 27%).

MS (ESI): mass calcd. for C₂₃H₂₃F₃N₆O₂, 472.5. m/z found, 473.2 [M+H]⁺. 1H NMR (400 MHz, Chloroform-d) δ 8.37 (d, J=7.2 Hz, 1H), 8.27 (s, 1H), 7.59 (s, 1H), 7.19 (s, 1H), 7.00 (s, 1H), 6.88 (dd, J=7.1, 1.9 Hz, 1H), 6.55 (s, 1H), 4.32 (d, J=9.7 Hz, 1H), 4.10 (d, J=9.7 Hz, 1H), 3.51 (s, 1H), 2.57 (s, 6H), 2.42 (s, 3H), 1.91 (bs, 1H), 1.48 (s, 3H).

### Example 264: (*R)-3-[[4-[2-[(2,6-dimethylpyrimidin-4-yl)amino]pyrazolo[1,5-a]pyridin-5-yl]-6-methyl-3-pyridyl]oxy]-1,1,1-trifluoro-2-methyl-propan-2-ol

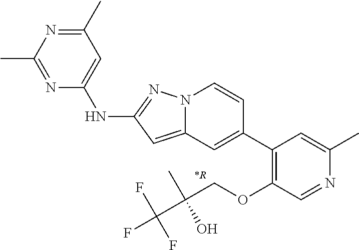

The second elute isomer from Example 263 was designated as (*R) isomer (175 mg, 27%). MS (ESI): mass calcd. for C₂₃H₂₃F₃N₆O₂, 472.5. m/z found, 473.2 [M+H]⁺. 1H NMR (400 MHz, Chloroform-d) δ 8.27 (d, J=7.2 Hz, 1H), 8.18 (s, 1H), 7.51 (d, J=1.9 Hz, 1H), 7.11 (s, 1H), 6.98 (s, 1H), 6.81 (dd, J=7.2, 2.0 Hz, 1H), 6.48 (s, 1H), 4.22 (d, J=9.7 Hz, 1H), 4.03-3.95 (m, 1H), 3.42 (s, 1H), 2.50 (d, J=3.6 Hz, 6H), 2.36 (s, 3H), 1.66 (bs, 1H), 1.37 (d, J=1.2 Hz, 3H).

### Example 265: 2-(cyclopropylmethyl)-6-[[5-[5-(2-hydroxy-2-methyl-propoxy)-2-methyl-4-pyridyl]pyrazolo[1,5-a]pyridin-2-yl]amino]-4-methyl-pyridazin-3-one

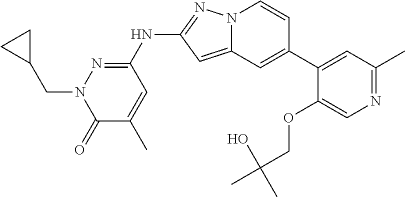 [SMILES: Cc1cc(-c2ccn3nc(Nc4cc(C)c(=O)n(CC5CC5)n4)cc3c2)c(OCC(C)(C)O)cn1]

Step A: 6-Chloro-2-(cyclopropylmethyl)-4-methylpyridazin-3(2H)-one. A 30 mL microwave vial was charged with 6-chloro-4-methylpyridazin-3(2H)-one (1.00 g, 6.92 mmol) and DMF (14 mL). At rt, cesium carbonate was added (5.64 g, 17.3 mmol) followed by (bromomethyl)cyclopropane (1.87 g, 13.8 mmol). The reaction mixture was stirred at rt overnight. The reaction mixture was then dissolved in EtOAc, washed with water and brine, and the organic layers were dried Na₂SO₄, and then filtered, concentrated to dryness under reduced pressure. The crude product was purified via flash column chromatography purified over silica gel (20-100% EtOAc/hexanes) to afford desired product (1.09 g, 79%). MS (ESI): mass calcd. for C₉H₁₁ClN₂O, 198.1. m/z found, 199.1 [M+H]⁺.

Step B: A 30 mL microwave vial containing a stir bar was charged 1-((4-(2-aminopyrazolo[1,5-a]pyridin-5-yl)-6-methylpyridin-3-yl)oxy)-2-methylpropan-2-ol (102 mg, 0.33 mmol), 2-(tert-butyl)-4-chloro-6-methylpyrimidine (71.4 mg, 0.36 mmol), tBuBrettPhos Pd G3 (14 mg, 0.02 mmol), cesium carbonate (319 mg, 0.98 mmol), and 1,4-dioxane (0.7 mL). The reaction mixture was degassed under vacuum with sonication for 2 minutes, then vented with argon. The vial was placed into a pre-heated plate at 80° C. for 16 h. The reaction mixture was then filtered, concentrated to dryness under reduced pressure to afford the crude product which was purified over basic ISCO (Gemini 5 uM NX-C18 110 angstrom LC column 150×21.2 mm; 10-100% ACN/20 mM aqueous NH₃ over 15 min; 40 mL/min) to afford the title compound (13.2 mg, 9%) as a white solid. MS (ESI): mass calcd. for C₂₆H₃₀N₆O₃, 474.6. m/z found, 475.3 [M+H]⁺. ¹H NMR (500 MHz, Methanol-d₄) δ 8.39 (dt, J=7.2, 0.9 Hz, 1H), 8.23 (s, 1H), 7.81 (dd, J=2.0, 0.9 Hz, 1H), 7.39 (s, 1H), 7.26 (q, J=1.2 Hz, 1H), 7.07 (dd, J=7.2, 2.0 Hz, 1H), 6.90 (d, J=0.9 Hz, 1H), 4.84 (s, 2H), 4.01 (d, J=7.1 Hz, 2H), 3.93 (s, 2H), 2.53 (s, 3H), 2.21 (d, J=1.3 Hz, 3H), 1.47-1.36 (m, 1H), 1.26 (s, 6H), 0.61-0.51 (m, 2H), 0.54-0.45 (m, 2H).

### Example 266: N-(2,6-dimethylpyrimidin-4-yl)-5-[2-methyl-5-(4-methylsulfonylcyclohexoxy)-4-pyridyl]pyrazolo[1,5-a]pyridin-2-amine

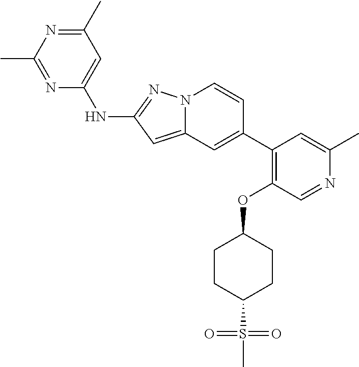

A 30 mL microwave vial was charged with 4-(methylsulfonyl)cyclohexan-1-ol (192 mg, 1.08 mmol) and 1-methylpyrrolidin-2-one (10 mL). Sodium hydride (60% dispersion in mineral oil, 86.1 mg, 2.15 mmol) was added. The reaction mixture was stirred at rt for 10 min. N-(2,6-Dimethylpyrimidin-4-yl)-5-(5-fluoro-2-methylpyridin-4-yl)pyrazolo[1,5-a]pyridin-2-amine (Intermediate 15, 250 mg, 0.72 mmol) was then added to the mixture. The resulting mixture was stirred at 80° C. for 16 hr. Cooled and quenched the reaction with CH₃OH. The crude was purified via prep. HPLC using 20-70% ACN-H₂O with 20 mM of NH₄OH as modifier. The pure fraction was lyophilized down to afford a white solid (258 mg, 71%). MS (ESI): mass calcd. for C₂₆H₃₀N₆O3S, 506.6. m/z found, [M+H]⁺. 1H NMR (500 MHz, Chloroform-d) δ 8.57 (s, 1H), 8.47 (dd, J=13.6, 7.1 Hz, 1H), 8.25 (d, J=10.7 Hz, 1H), 7.64-7.59 (m, 1H), 7.18 (d, J=6.1 Hz, 1H), 7.01 (s, 1H), 6.96-6.89 (m, 1H), 6.64 (s, 1H), 3.46 (s, 3H), 2.81 (d, J=2.6 Hz, 3H), 2.61-2.51 (m, 7H), 2.42 (d, J=2.8 Hz, 3H), 2.31-2.17 (m, 2H), 1.98 (dd, J=13.2, 3.7 Hz, 1H), 1.84-1.74 (m, 1H), 1.69-1.51 (m, 2H). The mixture of N-(2,6-dimethylpyrimidin-4-yl)-5-(2-methyl-5-((4-(methylsulfonyl)cyclohexyl)oxy)pyridin-4-yl)pyrazolo[1,5-a]pyridin-2-amine was separated via chiral chromatography (AS-H 2×25 cm, mobile phase: 40% 0.1% DEA in MeOH, 60% CO₂) to afford two peaks. The second elute isomer was designated as trans-isomer (106 mg, 29%). MS (ESI): mass calcd. for C₂₃H₂₃F₃N₆O₂, 472.5. m/z found, 473.2 [M+H]⁺. 1H NMR (500 MHz, Chloroform-d) δ 8.44 (d, J=7.2 Hz, 1H), 8.28 (s, 1H), 7.68-7.63 (m, 2H), 7.22 (s, 1H), 7.12 (s, 1H), 6.97 (dd, J=7.2, 1.9 Hz, 1H), 6.66 (s, 1H), 4.71 (q, J=2.8 Hz, 1H), 2.92-2.84 (m, 1H), 2.62 (s, 3H), 2.58 (s, 6H), 2.48 (s, 3H), 2.31-2.22 (m, 2H), 2.02 (dd, J=12.7, 3.7 Hz, 2H), 1.89-1.77 (m, 2H), 1.70-1.55 (m, 2H).

### Example 267: (rac-)-5-[5-(2-amino-3,3,3-trifluoro-2-methyl-propoxy)-2-methyl-4-pyridyl]-N-(2,6-dimethylpyrimidin-4-yl)pyrazolo[1,5-a]pyridin-2-amine

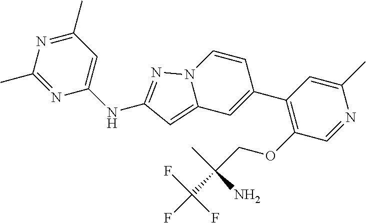

A 30 mL microwave vial was charged with 2-amino-3,3,3-trifluoro-2-methylpropan-1-ol HCl salt (193 mg, 1.08 mmol) and 1-methylpyrrolidin-2-one (10 mL). Sodium hydride (60% dispersion in mineral oil, 86.1 mg, 2.15 mmol) was added. The reaction mixture was stirred at rt for 10 min. N-(2,6-Dimethylpyrimidin-4-yl)-5-(5-fluoro-2-methylpyridin-4-yl)pyrazolo[1,5-a]pyridin-2-amine (Intermediate 15, 250 mg, 0.72 mmol) was then added to the mixture. The resulting mixture was stirred at 80° C. for 16 hr. Cooled and quenched the reaction with CH₃OH. The crude was purified via prep. HPLC using 20-70% ACN-H₂O with 20 mM of NH₄OH as modifier. The pure fraction was lyophilized down to afford a white solid (254 mg, 75%). MS (ESI): mass calcd. for C₂₃H₂₄F₃N₇O, 471.5. m/z found, 472.2 [M+H]⁺. ¹H NMR (500 MHz, Chloroform-d) δ 8.92-8.86 (m, 1H), 8.54-8.48 (m, 1H), 8.24 (s, 1H), 7.59 (d, J=1.9 Hz, 1H), 7.17 (s, 1H), 7.01 (s, 1H), 6.90 (dd, J=7.2, 1.9 Hz, 1H), 6.59 (s, 1H), 4.18 (d, J=9.4 Hz, 1H), 3.98-3.91 (m, 1H), 2.84 (s, 1H), 2.59 (s, 3H), 2.55 (s, 3H), 2.41 (s, 3H), 1.61 (s, 2H), 1.27 (s, 3H).

### Example 268: 4-[[5-[5-(2-cyano-2-methyl-propoxy)-2-methyl-4-pyridyl]pyrazolo[1,5-a]pyridin-2-yl]amino]-N-cyclopropyl-2,6-dimethoxy-benzamide

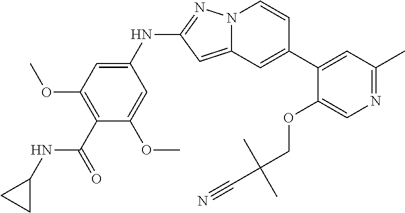 [SMILES: COc1cc(Nc2cc3cc(-c4cc(C)ncc4OCC(C)(C)C#N)ccn3n2)cc(OC)c1C(=O)NC1CC1]

The title compound was prepared using methods analogous to those described in Example 136 and using Intermediate 21 instead of Intermediate 6 and using 3-hydroxy-2,2-dimethylpropanenitrile instead of endo-7-hydroxy-3-oxa-9-azabicyclo[3.3.1]nonane hydrochloride. MS (ESI): mass calcd. for C₃₀H₃₂N₆O₄, 540.2. m/z found, 541.20 [M+H]⁺. ¹H NMR (600 MHz, Chloroform-d) δ 8.30 (d, J=7.0 Hz, 1H), 8.17 (s, 1H), 7.58 (d, J=1.9 Hz, 1H), 7.55-7.50 (m, 1H), 7.18 (s, 1H), 6.88 (dd, J=7.2, 2.0 Hz, 1H), 6.71 (s, OH), 6.64 (s, 2H), 6.15 (d, J=15.2 Hz, 2H), 3.96 (s, 2H), 3.77 (s, 1H), 3.72 (s, 5H), 2.95-2.88 (m, 1H), 2.54 (s, 3H), 1.38 (s, 6H), 0.78 (td, J=7.1, 5.2 Hz, 2H), 0.61-0.54 (m, 2H).

### Example 269: 3-[[4-[2-[(1,5-dimethyl-6-oxo-pyridazin-3-yl)amino]pyrazolo[1,5-a]pyridin-5-yl]-6-methyl-3-pyridyl]oxy]-2,2-dimethyl-propanenitrile

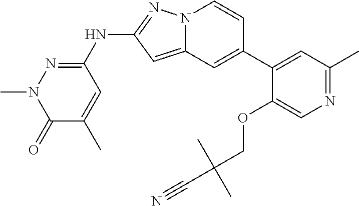

A 30 mL microwave vial was charged with 3-hydroxy-2,2-dimethylpropanenitrile (122 mg, 1.24 mmol) and 1-methylpyrrolidin-2-one (8 mL). Sodium hydride (60% dispersion in mineral oil, 99 mg, 2.47 mmol) was added. The reaction mixture was stirred at room temperature for 10 min. Intermediate 17 was then added to the mixture. The resulting mixture was stirred at 80° C. for 16 hr. Cooled and quenched the reaction with CH₃OH. The crude was purified via prep. HPLC using 20-70% ACN-H₂O with 20 mM of NH₄OH as modifier. The pure fraction was lyophilized down to afford a white solid (314 mg, 86%). MS (ESI): mass calcd. for C₂₄H₂₅N₇O₂, 443.5. m/z found, 444.2 [M+H]⁺. ¹H NMR (400 MHz, Chloroform-d) δ 8.34 (dd, J=7.2, 0.9 Hz, 1H), 8.23 (s, 1H), 7.82 (dd, J=9.9, 3.8 Hz, 1H), 7.69 (dd, J=2.0, 0.9 Hz, 1H), 7.22 (s, 1H), 7.14 (d, J=1.4 Hz, 1H), 7.01 (dd, J=7.2, 1.9 Hz, 1H), 6.76 (d, J=0.8 Hz, 1H), 4.00 (s, 2H), 3.78 (s, 3H), 2.58 (s, 3H), 2.24 (d, J=1.2 Hz, 3H), 1.41 (s, 6H).

### Example 270: (*R)-5-[5-(2-Amino-3,3,3-trifluoro-2-methyl-propoxy)-2-methyl-4-pyridyl]-N-(2,6-dimethylpyrimidin-4-yl)pyrazolo[1,5-a]pyridin-2-amine

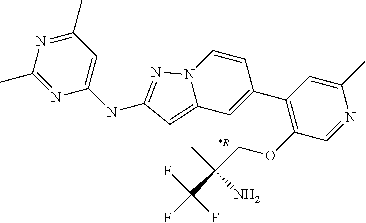

The mixture of 5-(5-(2-amino-3,3,3-trifluoro-2-methylpropoxy)-2-methylpyridin-4-yl)-N-(2,6-dimethylpyrimidin-4-yl)pyrazolo[1,5-a]pyridin-2-amine (Example 267) was separated via chiral SFC (Lux Cellulose-4, 2×25 cm, 40% 0.1% DEA in MeOH, 60% CO₂), followed by SFC (AS-H 2×25 cm, mobile phase: 40% 0.1% DEA in MeOH, 60% CO₂) to afford two peaks. The first elute isomer was designated as *R isomer (160 mg, 47%). MS (ESI): mass calcd. for C₂₃H₂₃F₃N₆O₂, 472.5. m/z found, 473.2 [M+H]⁺. 1H NMR (500 MHz, Chloroform-d) δ 8.44 (d, J=7.2 Hz, 1H), 8.28 (s, 1H), 7.68-7.63 (m, 2H), 7.22 (s, 1H), 7.12 (s, 1H), 6.97 (dd, J=7.2, 1.9 Hz, 1H), 6.66 (s, 1H), 4.71 (q, J=2.8 Hz, 1H), 2.92-2.84 (m, 1H), 2.62 (s, 3H), 2.58 (s, 6H), 2.48 (s, 3H), 2.31-2.22 (m, 2H), 2.02 (dd, J=12.7, 3.7 Hz, 2H), 1.89-1.77 (m, 2H), 1.70-1.55 (m, 2H).

### Example 271: N-(2,6-dimethylpyrimidin-4-yl)-5-[5-[(2-methoxyphenyl)methoxy]-2-methyl-4-pyridyl]pyrazolo[1,5-a]pyridin-2-amine

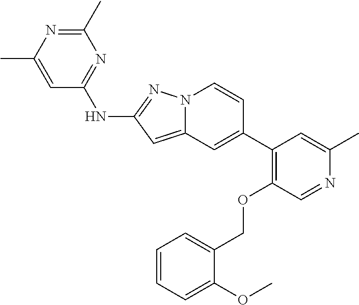

The title compound (54 mg, 15%) was prepared as described in Example 198 Example 198, except 2-methoxybenzyl alcohol (1.50 equiv.) was used instead of trans-1,4-cyclohexanediol. MS (ESI): mass calcd. for C₂₇H₂₆N₆O₂, 466.2. m/z found, 467.3 [M+H]⁺. ¹H NMR (500 MHz, DMSO-d6) δ 10.17 (s, 1H), 8.56 (dd, J=7.3, 1.0 Hz, 1H), 8.40 (s, 1H), 7.82 (dd, J=2.1, 1.0 Hz, 1H), 7.39-7.29 (m, 3H), 7.07-7.00 (m, 2H), 6.99 (s, 1H), 6.93 (td, J=7.4, 1.0 Hz, 1H), 6.84 (s, 1H), 5.21 (s, 2H), 3.83 (s, 3H), 2.46 (d, J=1.4 Hz, 6H), 2.30 (s, 3H).

### Example 272: 3-[[4-[2-[(2,6-dimethylpyrimidin-4-yl)amino]pyrazolo[1,5-a]pyridin-5-yl]-6-methyl-3-pyridyl]oxymethyl]phenol

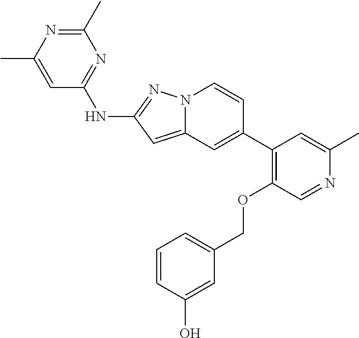

Step A: To a solution of 3-tert-butoxybenzyl alcohol (194 mg, 1.08 mmol, 1.50 equiv.) in DMA (2.20 mL) was added NaH (86 mg, 60% dispersion in mineral oil). The mixture allowed to stir at 23° C. for 5 min. To this was added Intermediate 15 (250 mg, 0.72 mmol), and the reaction was stirred at 80° C. for 2 hr. The reaction was cooled to 23° C. worked-up in sat. aq. NH4Cl (5 mL) and extracted with EtOAc (3×5 mL). The organic extracts were dried with magnesium sulfate, filtered and concentrated in vacuo. The crude material was purified by silica gel chromatography (0-10% MeOH in DCM) to give 5-(5-((3-(tert-butoxy)benzyl)oxy)-2-methylpyridin-4-yl)-N-(2,6-dimethylpyrimidin-4-yl)pyrazolo[1,5-a]pyridin-2-amine (206 mg, 56%).

Step B: 5-(5-((3-(tert-butoxy)benzyl)oxy)-2-methylpyridin-4-yl)-N-(2,6-dimethylpyrimidin-4-yl)pyrazolo[1,5-a]pyridin-2-amine was dissolved in dichloromethane (1.7 mL, 0.2 M) and trifluoroacetic acid (0.26 mL, 10.0 equiv.) and heated at 40° C. for 5 hours. The mixture was then concentrated in vacuo purified via prep HPLC (Basic ISCO, 10-80% AcN/20 mM aq NH₃) and lyophilized to yield the title compound as a white solid (43 mg, 16%). MS (ESI): mass calcd. for C₂₆H₂₄N₆O₂, 452.2. m/z found, 453.2 [M+H]⁺. ¹H NMR (500 MHz, DMSO-d6) δ 10.18 (s, 1H), 9.43 (s, 1H), 8.58 (dd, J=7.0, 1.0 Hz, 1H), 8.36 (s, 1H), 7.83 (dd, J=1.9, 0.9 Hz, 1H), 7.36 (s, 1H), 7.15 (t, J=7.8 Hz, 1H), 7.04 (dd, J=7.2, 2.0 Hz, 1H), 7.01 (s, 1H), 6.86 (s, 1H), 6.85-6.80 (m, 2H), 6.73-6.66 (m, 1H), 5.19 (s, 2H), 2.47-2.43 (m, 6H), 2.30 (s, 3H).

### Example 273: N-(2,6-dimethylpyrimidin-4-yl)-5-[5-[(3-ethyloxetan-3-yl)methoxy]-2-methyl-4-pyridyl]pyrazolo[1,5-a]pyridin-2-amine

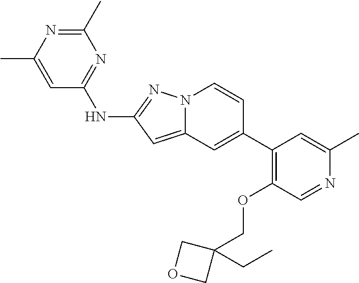

The title compound (63 mg, 49%) was prepared as described in Example 198, except 3-ethyl-3-oxetanemethanol (2.50 equiv.) was used instead of trans-1,4-cyclohexanediol. MS (ESI): mass calcd. for C₂₅H₂₈N₆O₂, 444.2. m/z found, 445.3 [M+H]⁺. ¹H NMR (600 MHz, DMSO-d6) δ 10.19 (s, 1H), 8.57 (dt, J=7.2, 0.9 Hz, 1H), 8.42 (s, 1H), 7.79 (dd, J=2.0, 0.9 Hz, 1H), 7.37 (s, 1H), 7.06-7.00 (m, 1H), 6.98 (dd, J=7.2, 2.0 Hz, 1H), 6.82 (s, 1H), 4.42 (d, J=6.0 Hz, 2H), 4.27 (d, J=6.0 Hz, 2H), 4.24 (s, 2H), 2.47 (s, 3H), 2.45 (s, 3H), 2.30 (s, 3H), 1.71 (q, J=7.5 Hz, 2H), 0.83 (t, J=7.5 Hz, 3H).

### Example 274: [3-[[4-[2-[(2,6-dimethylpyrimidin-4-yl)amino]pyrazolo[1,5-a]pyridin-5-yl]-6-methyl-3-pyridyl]oxymethyl]oxetan-3-yl]methanol

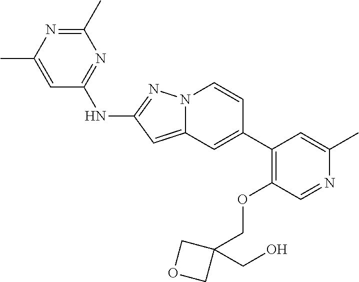

The title compound (32 mg, 25%) was prepared as described in Example 198, except oxetane-3,3-diyldimethanol (2.50 equiv.) was used instead of trans-1,4-cyclohexanediol. MS (ESI): mass calcd. for C₂₄H₂₆N₆O₃, 446.2. m/z found, 447.2 [M+H]⁺. ¹H NMR (600 MHz, DMSO-d6) δ 10.19 (s, 1H), 8.56 (dt, J=7.2, 0.9 Hz, 1H), 8.39 (s, 1H), 7.79 (dd, J=2.0, 0.9 Hz, 1H), 7.37 (s, 1H), 7.04 (s, 1H), 6.99 (dd, J=7.2, 2.0 Hz, 1H), 6.82 (s, 1H), 4.98 (t, J=5.4 Hz, 1H), 4.40 (d, J=6.0 Hz, 2H), 4.33 (d, J=6.0 Hz, 2H), 4.30 (s, 2H), 3.64 (d, J=5.4 Hz, 2H), 2.47 (s, 3H), 2.45 (s, 3H), 2.31-2.29 (m, 3H).

### Example 275: 3-[[4-[2-[(2,6-dimethylpyrimidin-4-yl)amino]pyrazolo[1,5-a]pyridin-5-yl]-6-methyl-3-pyridyl]oxymethyl]oxetane-3-carbonitrile

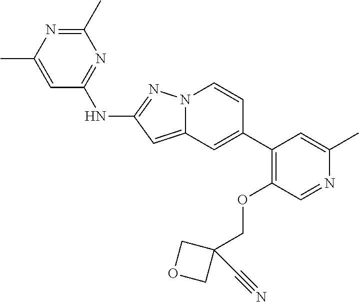

The title compound (31 mg, 16%) was prepared as described in Example 198, except 1-(hydroxymethyl)cyclobutene-1-carbonitrile (2.50 equiv.) was used instead of trans-1,4-cyclohexanediol. MS (ESI): mass calcd. for C₂₄H₂₃N₇O₂, 441.2. m/z found, 442.2 [M+H]⁺. ¹H NMR (500 MHz, DMSO-d6) δ 10.20 (s, 1H), 8.56 (dd, J=7.2, 0.9 Hz, 1H), 8.42 (s, 1H), 7.84 (dd, J=2.0, 0.9 Hz, 1H), 7.41 (s, 1H), 7.05-6.98 (m, 2H), 6.84 (s, 1H), 4.83 (d, J=6.6 Hz, 2H), 4.67 (s, 2H), 4.64 (d, J=6.6 Hz, 2H), 2.49 (s, 3H), 2.45 (s, 3H), 2.30 (s, 3H).

### Example 276: N-(2,6-dimethylpyrimidin-4-yl)-5-[2-methyl-5-[(3S)-pyrrolidin-3-yl]oxy-4-pyridyl]pyrazolo[1,5-a]pyridin-2-amine

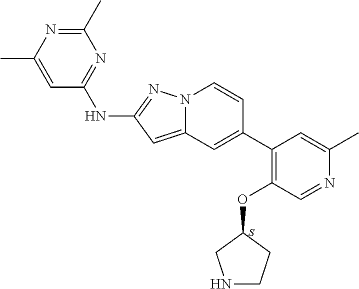

The title compound (129 mg, 85%) was prepared as described in Example 272, except tert-butyl (S)-3-hydroxypyrrolidine-1-carboxylate (2.50 equiv.) was used instead of 3-tert-butoxybenzyl alcohol in Step A and dichloromethane (0.60 mL) and trifluoroacetic acid (0.45 mL, 20.0 equiv.) at 23° C. for 30 minutes was used in Step B. MS (ESI): mass calcd. for C₂₃H₂₅N₇O, 415.2. m/z found, 416.2 [M+H]⁺. ¹H NMR (500 MHz, DMSO-d6) δ 10.19 (s, 1H), 8.56 (d, J=7.2 Hz, 1H), 8.30 (s, 1H), 7.80 (dd, J=1.9, 0.9 Hz, 1H), 7.34 (s, 1H), 7.09-6.93 (m, 2H), 6.85 (s, 1H), 5.00-4.91 (m, 1H), 3.03 (dd, J=12.3, 5.1 Hz, 1H), 2.86 (dd, J=15.3, 7.7 Hz, 2H), 2.78-2.70 (m, 1H), 2.46 (s, 6H), 2.30 (s, 3H), 1.98 (dq, J=14.3, 7.4 Hz, 1H), 1.77 (dt, J=12.5, 5.7 Hz, 1H).

### Example 277: N-[5-[2-methyl-5-[(3S)-pyrrolidin-3-yl]oxy-4-pyridyl]pyrazolo[1,5-a]pyridin-2-yl]cyclopropanecarboxamide

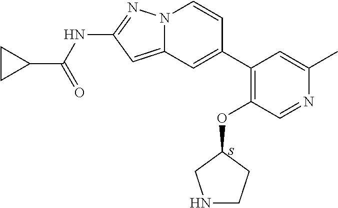

The title compound (41 mg, 22%) was prepared as described in Example 276, except Intermediate 6 was used instead of Intermediate 15 in Step A. MS (ESI): mass calcd. for C₂₁H₂₃N₅O₂, 377.2. m/z found, 378.2 [M+H]⁺. ¹H NMR (500 MHz, DMSO-d6) δ 11.09 (s, 1H), 8.52 (d, J=7.3 Hz, 1H), 8.28 (s, 1H), 7.80 (s, 1H), 7.32 (s, 1H), 7.05-7.00 (m, 1H), 6.89 (s, 1H), 4.97 (d, J=5.6 Hz, 1H), 3.03 (t, J=8.1 Hz, 1H), 2.87 (t, J=13.7 Hz, 2H), 2.76 (s, 1H), 2.44 (s, 3H), 2.01-1.88 (m, 2H), 1.83-1.70 (m, 1H), 0.88-0.68 (m, 4H).

### Example 278: N-(2,6-dimethylpyrimidin-4-yl)-5-[5-[(3R,4S)-4-fluoropyrrolidin-3-yl]oxy-2-methyl-4-pyridyl]pyrazolo[1,5-a]pyridin-2-amine

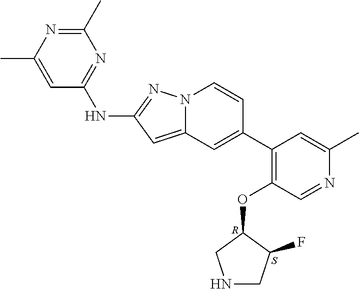

The title compound (34 mg, 18%) was prepared as described in Example 280, except tert-butyl (3S,4R)-3-fluoro-4-hydroxypyrrolidine-1-carboxylate was used instead of tert-butyl (S)-3-hydroxypyrrolidine-1-carboxylate in Step A. MS (ESI): mass calcd. for C₂₃H₂₄FN₇O, 433.2. m/z found, 434.2 [M+H]⁺. ¹H NMR (500 MHz, DMSO-d6) δ 10.19 (s, 1H), 8.60-8.55 (m, 1H), 8.45 (s, 1H), 7.83 (dd, J=2.0, 0.9 Hz, 1H), 7.38 (s, 1H), 7.05 (dd, J=7.3, 2.0 Hz, 1H), 7.01 (s, 1H), 6.85 (s, 1H), 5.35-5.18 (m, 1H), 4.91-4.79 (m, 1H), 3.27-3.14 (m, 2H), 2.96 (dd, J=26.1, 13.2 Hz, 1H), 2.83-2.71 (m, 1H), 2.47 (s, 3H), 2.46 (s, 3H), 2.30 (s, 3H).

### Example 279: N-[5-[5-[(3R,4S)-4-fluoropyrrolidin-3-yl]oxy-2-methyl-4-pyridyl]pyrazolo[1,5-a]pyridin-2-yl]cyclopropanecarboxamide

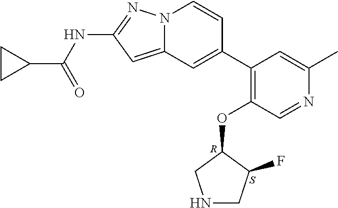

The title compound (12 mg, 9%) was prepared as described in Example 280, except Intermediate 6 was used instead of Intermediate 15 and tert-butyl (3S,4R)-3-fluoro-4-hydroxypyrrolidine-1-carboxylate was used instead of tert-butyl (S)-3-hydroxypyrrolidine-1-carboxylate in Step A. MS (ESI): mass calcd. for C₂₁H₂₂FN₅O₂, 395.2. m/z found, 396.2 [M+H]⁺. ¹H NMR (500 MHz, DMSO-d6) δ 11.07 (s, 1H), 8.56 (dd, J=7.2, 1.0 Hz, 1H), 8.44 (s, 1H), 7.84 (dd, J=2.0, 0.9 Hz, 1H), 7.37 (s, 1H), 7.08 (dd, J=7.2, 2.0 Hz, 1H), 6.88 (s, 1H), 5.34-5.16 (m, 1H), 4.86 (dtd, J=18.1, 7.1, 4.4 Hz, 1H), 3.26-3.12 (m, 2H), 3.00-2.89 (m, 1H), 2.79 (dd, J=11.3, 7.3 Hz, 1H), 2.47 (s, 3H), 1.93 (tt, J=7.6, 4.7 Hz, 1H), 0.87-0.77 (m, 4H).

### Example 280: N-[5-[2-prop-1-ynyl-5-[(3S)-pyrrolidin-3-yl]oxy-4-pyridyl]pyrazolo[1,5-a]pyridin-2-yl]cyclopropanecarboxamide

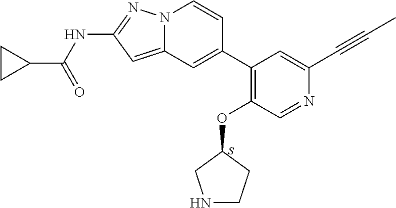

Step A: To a solution of tert-butyl (S)-3-hydroxypyrrolidine-1-carboxylate (92 mg, 0.49 mmol, 1.10 equiv.) in DMA (2.60 mL) was added NaH (40 mg, 60% dispersion in mineral oil). The mixture was stirred at 23° C. for 5 min. To this was added Intermediate 11 (150 mg, 0.45 mmol), and the reaction was stirred at 23° C. for 20 minutes. Additional NaH (20 mg, 60% dispersion in mineral oil) was added at 23° C. and the resulting solution stirred for 3 hours. The reaction was quenched with sat. aq. NH₄Cl (4 mL) and extracted with EtOAc (3×5 mL). The organic extracts were dried with magnesium sulfate, filtered and concentrated in vacuo. The crude material was purified by silica gel chromatography (0-10% MeOH in DCM) to give tert-butyl (S)-3-((4-(2-(cyclopropanecarboxamido)pyrazolo[1,5-a]pyridin-5-yl)-6-(prop-1-yn-1-yl)pyridin-3-yl)oxy)pyrrolidine-1-carboxylate (90 mg, 40%).

Step B: tert-butyl (S)-3-((4-(2-(cyclopropanecarboxamido)pyrazolo[1,5-a]pyridin-5-yl)-6-(prop-1-yn-1-yl)pyridin-3-yl)oxy)pyrrolidine-1-carboxylate was dissolved in dichloromethane (0.3 mL) and trifluoroacetic acid (0.3 mL, 20.0 equiv.) and stirred for 30 minutes. The mixture was then concentrated in vacuo purified via prep HPLC (Basic ISCO, 10-80% AcN/20 mM aq NH₃) and lyophilized to yield the title compound as a white solid (30 mg, 16%). MS (ESI): mass calcd. for C₂₃H₂₃N₅O₂, 401.2. m/z found, 402.1 [M+H]⁺. ¹H NMR (500 MHz, DMSO-d6) δ 11.08 (s, 1H), 8.53 (dd, J=7.2, 1.0 Hz, 1H), 8.38 (s, 1H), 7.85 (d, J=2.2 Hz, 1H), 7.52 (s, 1H), 7.06 (dd, J=7.3, 2.0 Hz, 1H), 6.89 (s, 1H), 5.12-5.05 (m, 1H), 3.06 (dd, J=12.5, 5.1 Hz, 1H), 2.94-2.83 (m, 2H), 2.77 (ddd, J=10.7, 8.1, 4.8 Hz, 1H), 2.06 (s, 3H), 2.05-1.99 (m, 1H), 1.96-1.89 (m, 1H), 1.83-1.75 (m, 1H), 0.82 (tt, J=7.9, 3.0 Hz, 4H).

### Example 281: N-[5-[5-[(3R,4S)-4-fluoropyrrolidin-3-yl]oxy-2-prop-1-ynyl-4-pyridyl]pyrazolo[1,5-a]pyridin-2-yl]cyclopropanecarboxamide

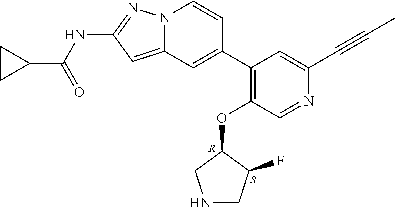

The title compound (57 mg, 27%) was prepared as described in Example 280, except tert-butyl (3S,4R)-3-fluoro-4-hydroxypyrrolidine-1-carboxylate was used instead of tert-butyl (S)-3-hydroxypyrrolidine-1-carboxylate in Step A. MS (ESI): mass calcd. for C₂₃H₂₂FN₅O₂, 419.2. m/z found, 420.1 [M+H]⁺. ¹H NMR (500 MHz, DMSO-d6) δ 11.09 (s, 1H), 8.59-8.52 (m, 2H), 7.88 (d, J=1.9 Hz, 1H), 7.55 (s, 1H), 7.09 (dd, J=7.3, 2.1 Hz, 1H), 6.88 (s, 1H), 5.39-5.21 (m, 1H), 5.03-4.91 (m, 1H), 3.29-3.10 (m, 2H), 3.02-2.88 (m, 1H), 2.86-2.76 (m, 1H), 2.07 (s, 3H), 1.93 (tt, J=7.9, 4.8 Hz, 1H), 0.88-0.76 (m, 4H).

### Example 282: 6-[[5-[5-(2-hydroxy-2-methyl-propoxy)-2-methyl-4-pyridyl]pyrazolo[1,5-a]pyridin-2-yl]amino]-2-isobutyl-4-methyl-pyridazin-3-one

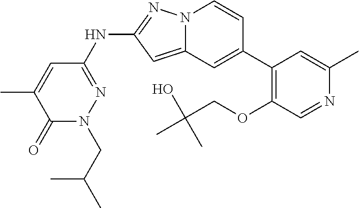

Step A: 6-chloro-2-isobutyl-4-methylpyridazin-3(2H)-one. To a mixture of 6-chloro-4-methylpyridazin-3(2h)-one (1 g, 6.92 mmol) and Cs₂CO₃ (5.64 g, 17.3 mmol) in DMF (13.8 mL, 6.92 mmol) was added 1-chloro-2-methylpropane (1.28 g, 13.8 mmol). The mixture was stirred at room temp for 16 hr, then worked-up in a separatory funnel with ethyl acetate/water/brine. The organic extracts were dried with Na₂SO₄, then purified via FCC (20% ethyl acetate in hexanes) to give 665 mg (48%) of the title compound.

Step B: 6-[[5-[5-(2-hydroxy-2-methyl-propoxy)-2-methyl-4-pyridyl]pyrazolo[1,5-a]pyridin-2-yl]amino]-2-isobutyl-4-methyl-pyridazin-3-one. The title compound (40 mg, 24%) was prepared as described in Example 224, except 1-((4-(2-aminopyrazolo[1,5-a]pyridin-5-yl)-6-methylpyridin-3-yl)oxy)-2-methylpropan-2-ol (Intermediate 20, 110 mg, 0.35 mmol) was used instead of (1r,4r)-4-((4-(2-aminopyrazolo[1,5-a]pyridin-5-yl)-6-methylpyridin-3-yl)oxy)cyclohexan-1-01 (Intermediate 12) and 6-chloro-2-isobutyl-4-methylpyridazin-3(2H)-one (78 mg, 0.39 mmol) was used instead of 4-chloro-2-ethyl-6-methylpyrimidine. MS (ESI): mass calcd. for C₂₆H₃₂N₆O₃, 476.3. m/z found, 477.3 [M+H]⁺. ¹H NMR (500 MHz, DMSO-d6) δ 9.74 (s, 1H), 8.56-8.51 (m, 1H), 8.30 (s, 1H), 7.91-7.85 (m, 1H), 7.39 (s, 1H), 7.25-7.17 (m, 1H), 7.08 (dd, J=7.2, 2.0 Hz, 1H), 6.86-6.78 (m, 1H), 4.62 (s, 1H), 3.87 (s, 2H), 3.84 (d, J=7.3 Hz, 2H), 2.46 (s, 3H), 2.30-2.20 (m, 1H), 2.09 (d, J=1.3 Hz, 3H), 1.16 (s, 6H), 0.93 (d, J=6.7 Hz, 6H).

### Example 283: 2-[2-(difluoromethoxy)ethyl]-6-[[5-[5-(4-hydroxycyclohexoxy)-2-methyl-4-pyridyl]pyrazolo[1,5-a]pyridin-2-yl]amino]-4-methyl-pyridazin-3-one

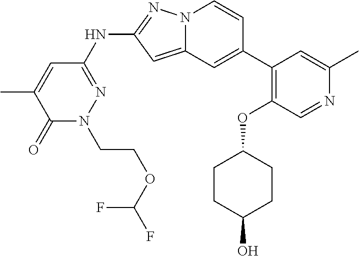

Step A. 6-chloro-2-(2-(difluoromethoxy)ethyl)-4-methylpyridazin-3(2H)-one. The title compound (1.15 g, 70%) was prepared as described in Example 282, Step A, except 1-bromo-2-(difluoromethoxy)ethane (1.63 g, 9.34 mmol) was used instead of 1-chloro-2-methylpropane.

Step B. 2-[2-(difluoromethoxy)ethyl]-6-[[5-[5-(4-hydroxycyclohexoxy)-2-methyl-4-pyridyl]pyrazolo[1,5-a]pyridin-2-yl]amino]-4-methyl-pyridazin-3-one. The title compound (95 mg, 57%) was prepared as described in Example 224, except 6-chloro-2-(2-(difluoromethoxy)ethyl)-4-methylpyridazin-3(2H)-one (81 mg, 0.34 mmol) was used instead of 4-chloro-2-ethyl-6-methylpyrimidine. MS (ESI): mass calcd. for C₂₇H₃₀F₂N₆O₄, 540.2. m/z found, 541.3 [M+H]⁺. ¹H NMR (400 MHz, DMSO-d6) δ 9.79 (s, 1H), 8.55-8.44 (m, 1H), 8.35 (s, 1H), 7.81-7.67 (m, 1H), 7.35 (s, 1H), 7.27-7.18 (m, 1H), 7.00 (dd, J=7.2, 2.0 Hz, 1H), 6.91-6.48 (m, 2H), 4.51 (s, 1H), 4.44-4.35 (m, 1H), 4.33-4.21 (m, 4H), 3.55-3.43 (m, 1H), 2.46 (s, 3H), 2.10 (d, J=1.3 Hz, 3H), 2.03-1.91 (m, 2H), 1.79-1.67 (m, 2H), 1.49-1.34 (m, 2H), 1.33-1.20 (m, 2H).

### Example 284: 2-[2-(difluoromethoxy)ethyl]-6-[[5-[5-(2-hydroxy-2-methyl-propoxy)-2-methyl-4-pyridyl]pyrazolo[1,5-a]pyridin-2-yl]amino]-4-methyl-pyridazin-3-one

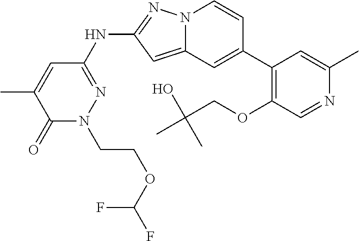

The title compound (97 mg, 54%) was prepared as described in Example 283, except 1-((4-(2-aminopyrazolo[1,5-a]pyridin-5-yl)-6-methylpyridin-3-yl)oxy)-2-methylpropan-2-ol (Intermediate 20, 110 mg, 0.35 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) in Step B. MS (ESI): mass calcd. for C₂₅H₂₈F₂N₆O₄, 514.2. m/z found, 515.3 [M+H]⁺. ¹H NMR (400 MHz, DMSO-d6) δ 9.79 (s, 1H), 8.58-8.49 (m, 1H), 8.31 (s, 1H), 7.86-7.80 (m, 1H), 7.37 (s, 1H), 7.24 (d, J=1.4 Hz, 1H), 7.07 (dd, J=7.2, 2.0 Hz, 1H), 6.91-6.45 (m, 2H), 4.61 (s, 1H), 4.34-4.22 (m, 4H), 3.87 (s, 2H), 2.47 (s, 3H), 2.12-2.08 (m, 3H), 1.15 (s, 6H).

### Example 285: 2-methyl-1-[[6-methyl-4-[2-[(6-methyl-2-pyrrolidin-1-yl-pyrimidin-4-yl)amino]pyrazolo[1,5-a]pyridin-5-yl]-3-pyridyl]oxy]propan-2-ol

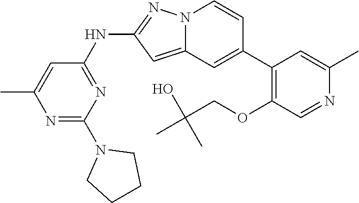

The title compound (13 mg, 7%) was prepared as described in Example 224, except 1-((4-(2-aminopyrazolo[1,5-a]pyridin-5-yl)-6-methylpyridin-3-yl)oxy)-2-methylpropan-2-ol (Intermediate 20, 110 mg, 0.35 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) and 4-chloro-6-methyl-2-pyrrolidin-1-ylpyrimidine (77 mg, 0.39 mmol) was used instead of 4-chloro-2-ethyl-6-methylpyrimidine. MS (ESI): mass calcd. for C₂₆H₃₁N₇O₂, 473.3. m/z found, 474.3 [M+H]⁺. ¹H NMR (400 MHz, DMSO-d6) δ 9.88 (s, 1H), 8.57-8.51 (m, 1H), 8.30 (s, 1H), 7.91-7.87 (m, 1H), 7.40 (s, 1H), 7.10-7.05 (m, 2H), 6.17 (s, 1H), 4.62 (s, 1H), 3.87 (s, 2H), 3.59-3.47 (m, 4H), 2.46 (s, 3H), 2.16 (s, 3H), 1.97-1.90 (m, 4H), 1.16 (s, 6H).

### Example 286: 2-methyl-1-[[6-methyl-4-[2-[(6-methyl-2-morpholino-pyrimidin-4-yl)amino]pyrazolo[1,5-a]pyridin-5-yl]-3-pyridyl]oxy]propan-2-ol

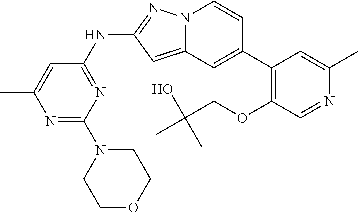

The title compound (77 mg, 45%) was prepared as described in Example 224, except 1-((4-(2-aminopyrazolo[1,5-a]pyridin-5-yl)-6-methylpyridin-3-yl)oxy)-2-methylpropan-2-ol (Intermediate 20, 110 mg, 0.35 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) and 4-(4-chloro-6-methyl-2-pyrimidinyl)morpholine (83 mg, 0.39 mmol) was used instead of 4-chloro-2-ethyl-6-methylpyrimidine. MS (ESI): mass calcd. for C₂₆H₃₁N₇O₃, 489.2. m/z found, 490.3 [M+H]⁺. ¹H NMR (400 MHz, DMSO-d6) δ 9.97 (s, 1H), 8.60-8.52 (m, 1H), 8.31 (s, 1H), 7.93-7.88 (m, 1H), 7.39 (s, 1H), 7.07 (dd, J=7.2, 2.0 Hz, 1H), 6.87 (s, 1H), 6.31 (s, 1H), 4.62 (s, 1H), 3.87 (s, 2H), 3.75-3.64 (m, 8H), 2.47 (s, 3H), 2.18 (s, 3H), 1.16 (s, 6H).

### Example 287: 4-[[6-methyl-4-[2-[(6-methyl-2-pyrrolidin-1-yl-pyrimidin-4-yl)amino]pyrazolo[1,5-a]pyridin-5-yl]-3-pyridyl]oxy]cyclohexanol

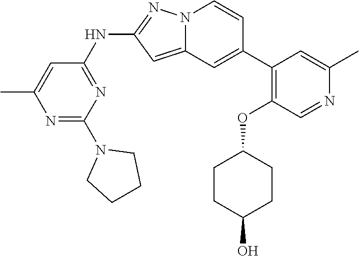

The title compound (47 mg, 16%) was prepared as described in Example 224, except 4-chloro-6-methyl-2-pyrrolidin-1-ylpyrimidine (129 mg, 0.65 mmol) was used instead of 4-chloro-2-ethyl-6-methylpyrimidine. MS (ESI): mass calcd. for C₂₈H₃₃N₇O₂, 499.3. m/z found, 500.3 [M+H]⁺. ¹H NMR (600 MHz, DMSO-d6) δ 9.87 (s, 1H), 8.54-8.50 (m, 1H), 8.34 (s, 1H), 7.79-7.74 (m, 1H), 7.37 (s, 1H), 7.06 (s, 1H), 7.00 (dd, J=7.2, 2.0 Hz, 1H), 6.18 (s, 1H), 4.50 (d, J=3.9 Hz, 1H), 4.45-4.36 (m, 1H), 3.62-3.44 (m, 5H), 2.45 (s, 3H), 2.16 (s, 3H), 2.00-1.90 (m, 6H), 1.76-1.70 (m, 2H), 1.46-1.37 (m, 2H), 1.33-1.24 (m, 2H).

### Example 288: 4-[[6-methyl-4-[2-[(6-methyl-2-morpholino-pyrimidin-4-yl)amino]pyrazolo[1,5-a]pyridin-5-yl]-3-pyridyl]oxy]cyclohexanol

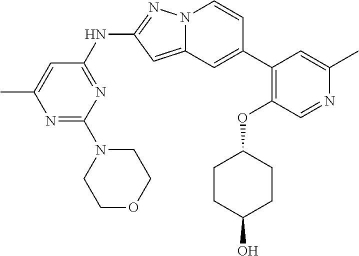

The title compound (58 mg, 19%) was prepared as described in Example 224, except 4-(4-chloro-6-methyl-2-pyrimidinyl)morpholine (139 mg, 0.65 mmol) was used instead of 4-chloro-2-ethyl-6-methylpyrimidine. MS (ESI): mass calcd. for C₂₈H₃₃N₇O₃, 515.3. m/z found, 516.3 [M+H]⁺. ¹H NMR (600 MHz, DMSO-d6) δ 9.96 (s, 1H), 8.54 (dd, J=7.2, 0.9 Hz, 1H), 8.35 (s, 1H), 7.81-7.75 (m, 1H), 7.36 (s, 1H), 7.01 (dd, J=7.2, 2.0 Hz, 1H), 6.86 (s, 1H), 6.32 (s, 1H), 4.51 (d, J=3.9 Hz, 1H), 4.44-4.37 (m, 1H), 3.73-3.70 (m, 4H), 3.69-3.66 (m, 4H), 3.53-3.46 (m, 1H), 2.46 (s, 3H), 2.18 (s, 3H), 2.00-1.92 (m, 2H), 1.77-1.69 (m, 2H), 1.46-1.37 (m, 2H), 1.32-1.23 (m, 2H).

### Example 289: 4-[[6-methyl-4-[2-(5H-pyrrolo[2,3-b]pyrazin-3-ylamino)pyrazolo[1,5-a]pyridin-5-yl]-3-pyridyl]oxy]cyclohexanol

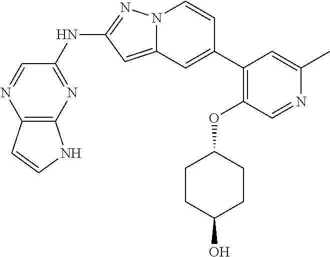 [SMILES: Cc1cc(-c2ccn3nc(Nc4cnc5cc[nH]c5n4)cc3c2)c(O[C@H]2CC[C@H](O)CC2)cn1]

The title compound (24 mg, 39%) was prepared as described in Example 224, except 3-chloro-5h-pyrrolo[2,3-b]pyrazine (22 mg, 0.15 mmol) was used instead of 4-chloro-2-ethyl-6-methylpyrimidine. MS (ESI): mass calcd. for C₂₅H₂₅N₇O₂, 455.2. m/z found, 456.3 [M+H]⁺. ¹H NMR (600 MHz, DMSO-d6) δ 11.54 (s, 1H), 10.08 (s, 1H), 8.59-8.53 (m, 1H), 8.37 (d, J=10.6 Hz, 2H), 7.78-7.73 (m, 1H), 7.41 (d, J=3.6 Hz, 1H), 7.35 (s, 1H), 7.09-7.05 (m, 1H), 6.98 (dd, J=7.1, 2.0 Hz, 1H), 6.48 (d, J=3.5 Hz, 1H), 4.51 (d, J=3.9 Hz, 1H), 4.47-4.38 (m, 1H), 3.54-3.47 (m, 1H), 2.46 (s, 3H), 2.02-1.94 (m, 2H), 1.77-1.71 (m, 2H), 1.46-1.37 (m, 2H), 1.33-1.24 (m, 2H).

### Example 290: (1r,4r)-4-((6-methyl-4-(2-(pyridin-3-ylamino)pyrazolo[1,5-a]pyridin-5-yl)pyridin-3-yl)oxy)cyclohexan-1-ol

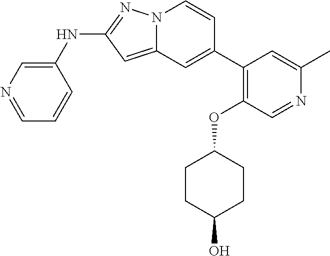 [SMILES: Cc1cc(-c2ccn3nc(Nc4cccnc4)cc3c2)c(O[C@H]2CC[C@H](O)CC2)cn1]

The title compound (31 mg, 25%) was prepared as described in Example 224, except 3-bromopyridine was used instead of 4-chloro-2-ethyl-6-methylpyrimidine. MS (ESI): mass calcd. for C₂₄H₂₅N₅O₂, 415.20. m/z found, 416.2 [M+H]⁺. 1H NMR (600 MHz, DMSO-d6) δ 9.19 (s, 1H), 8.71 (d, J=2.7 Hz, 1H), 8.56 (dt, J=7.2, 0.9 Hz, 1H), 8.35 (s, 1H), 8.06-7.98 (m, 2H), 7.68 (dd, J=2.0, 0.9 Hz, 1H), 7.41-7.35 (m, 1H), 7.33 (s, 1H), 7.28 (dd, J=8.3, 4.6 Hz, 1H), 6.92 (dd, J=7.2, 2.0 Hz, 1H), 6.16 (d, J=0.8 Hz, 1H), 4.49 (s, 1H), 4.40 (dq, J=8.9, 4.9, 4.3 Hz, 1H), 3.53-3.46 (m, 1H), 2.45 (s, 3H), 1.97 (t, J=8.8 Hz, 2H), 1.78-1.65 (m, 2H), 1.47-1.35 (m, 2H), 1.32-1.23 (m, 2H).

### Example 291: 2-methyl-1-[[6-methyl-4-[2-(5H-pyrrolo[2,3-b]pyrazin-3-ylamino)pyrazolo[1,5-a]pyridin-5-yl]-3-pyridyl]oxy]propan-2-ol

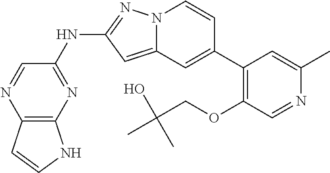 [SMILES: Cc1cc(-c2ccn3nc(Nc4cnc5cc[nH]c5n4)cc3c2)c(OCC(C)(C)O)cn1]

The title compound (14 mg, 10%) was prepared as described in Example 224, except 1-((4-(2-aminopyrazolo[1,5-a]pyridin-5-yl)-6-methylpyridin-3-yl)oxy)-2-methylpropan-2-ol (Intermediate 20, 100 mg, 0.32 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) and 3-chloro-5h-pyrrolo[2,3-b]pyrazine (54 mg, 0.35 mmol) was used instead of 4-chloro-2-ethyl-6-methylpyrimidine. MS (ESI): mass calcd. for C₂₃H₂₃N₇O₂, 429.2. m/z found, 430.2 [M+H]⁺. ¹H NMR (600 MHz, DMSO-d6) δ 11.56 (s, 1H), 10.08 (s, 1H), 8.61-8.55 (m, 1H), 8.38 (s, 1H), 8.32 (s, 1H), 7.88-7.82 (m, 1H), 7.42-7.39 (m, 1H), 7.37 (s, 1H), 7.06-7.03 (m, 2H), 6.48 (dd, J=3.6, 1.5 Hz, 1H), 4.63 (s, 1H), 3.88 (s, 2H), 2.47 (s, 3H), 1.17 (s, 6H).

### Example 292: 2-methyl-1-[[6-methyl-4-[2-[(1-methylpyrazolo[3,4-b]pyridin-3-yl)amino]pyrazolo[1,5-a]pyridin-5-yl]-3-pyridyl]oxy]propan-2-ol

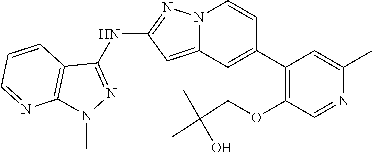 [SMILES: Cc1cc(-c2ccn3nc(Nc4nn(C)c5ncccc45)cc3c2)c(OCC(C)(C)O)cn1]

The title compound (123 mg, 62%) was prepared as described in Example 224, except 1-((4-(2-aminopyrazolo[1,5-a]pyridin-5-yl)-6-methylpyridin-3-yl)oxy)-2-methylpropan-2-ol (Intermediate 20, 140 mg, 0.45 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) and 3-bromo-1-methyl-1h-pyrazolo[3,4-b]pyridine (105 mg, 0.49 mmol) was used instead of 4-chloro-2-ethyl-6-methylpyrimidine. MS (ESI): mass calcd. for C₂₄H₂₅N₇O₂, 443.2. m/z found, 444.3 [M+H]⁺. ¹H NMR (500 MHz, DMSO-d6) δ 10.16 (s, 1H), 8.56-8.53 (m, 1H), 8.53-8.48 (m, 2H), 8.32 (s, 1H), 7.89-7.86 (m, 1H), 7.36 (s, 1H), 7.10 (dd, J=8.0, 4.6 Hz, 1H), 7.05 (dd, J=7.2, 2.0 Hz, 1H), 6.99-6.95 (m, 1H), 4.64 (s, 1H), 3.96 (s, 3H), 3.88 (s, 2H), 2.47 (s, 3H), 1.17 (s, 6H).

### Example 293: 2-ethyl-6-[[5-[5-[[(2R,3S)-3-hydroxytetrahydrofuran-2-yl]methoxy]-2-methyl-4-pyridyl]pyrazolo[1,5-a]pyridin-2-yl]amino]-4-methyl-pyridazin-3-one

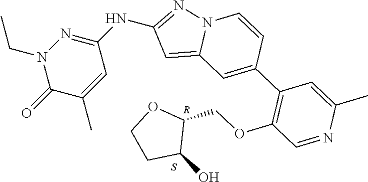

The title compound (95 mg, 66%) was prepared as described in Example 224, except (2R,3S)-2-(((4-(2-aminopyrazolo[1,5-a]pyridin-5-yl)-6-methylpyridin-3-yl)oxy)methyl)tetrahydrofuran-3-ol (Intermediate 25, 102 mg, 0.3 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) and 6-chloro-2-ethyl-4-methylpyridazin-3(2H)-one (Example 247, Step A, 57 mg, 0.33 mmol) was used instead of 4-chloro-2-ethyl-6-methylpyrimidine. MS (ESI): mass calcd. for C₂₅H₂₈N₆O₄, 476.2. m/z found, 477.2 [M+H]⁺. ¹H NMR (500 MHz, DMSO-d6) δ 9.75 (s, 1H), 8.57-8.51 (m, 1H), 8.34 (s, 1H), 7.89-7.83 (m, 1H), 7.38 (s, 1H), 7.24-7.19 (m, 1H), 7.04 (dd, J=7.2, 2.0 Hz, 1H), 6.87-6.81 (m, 1H), 5.05 (s, 1H), 4.21-4.11 (m, 3H), 4.10-3.99 (m, 2H), 3.89-3.76 (m, 3H), 2.46 (s, 3H), 2.09 (d, J=1.3 Hz, 3H), 1.99-1.90 (m, 1H), 1.75-1.65 (m, 1H), 1.32 (t, J=7.2 Hz, 3H).

### Example 294: (2R,3S)-2-[[6-methyl-4-[2-[(6-methyl-2-morpholino-pyrimidin-4-yl)amino]pyrazolo[1,5-a]pyridin-5-yl]-3-pyridyl]oxymethyl]tetrahydrofuran-3-ol

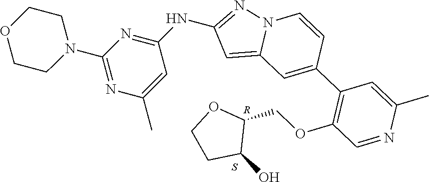

The title compound (96 mg, 62%) was prepared as described in Example 224, except (2R,3S)-2-(((4-(2-aminopyrazolo[1,5-a]pyridin-5-yl)-6-methylpyridin-3-yl)oxy)methyl)tetrahydrofuran-3-ol (Intermediate 25, 102 mg, 0.3 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) and 4-(4-chloro-6-methyl-2-pyrimidinyl)morpholine (70 mg, 0.33 mmol) was used instead of 4-chloro-2-ethyl-6-methylpyrimidine. MS (ESI): mass calcd. for C₂₇H₃₁N₇O₄, 517.2. m/z found, 518.2 [M+H]⁺. ¹H NMR (400 MHz, DMSO-d6) δ 9.97 (s, 1H), 8.60-8.51 (m, 1H), 8.34 (s, 1H), 7.92-7.81 (m, 1H), 7.38 (s, 1H), 7.03 (dd, J=7.2, 2.0 Hz, 1H), 6.87 (s, 1H), 6.31 (s, 1H), 5.04 (s, 1H), 4.23-4.08 (m, 3H), 3.90-3.74 (m, 3H), 3.74-3.65 (m, 8H), 2.46 (s, 3H), 2.18 (s, 3H), 2.00-1.89 (m, 1H), 1.74-1.65 (m, 1H).

### Example 295: (2R,3S)-2-[[4-[2-[[5-(difluoromethyl)pyrazin-2-yl]amino]pyrazolo[1,5-a]pyridin-5-yl]-6-methyl-3-pyridyl]oxymethyl]tetrahydrofuran-3-ol

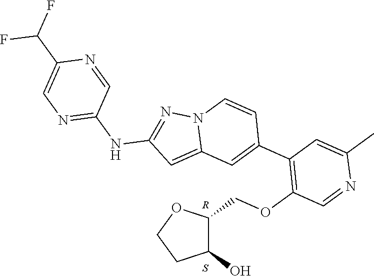

The title compound (11 mg, 6%) was prepared as described in Example 224, except (2R,3S)-2-(((4-(2-aminopyrazolo[1,5-a]pyridin-5-yl)-6-methylpyridin-3-yl)oxy)methyl)tetrahydrofuran-3-ol (Intermediate 25, 140 mg, 0.41 mmol) was used instead of (1r,4r)-4-((4-(2-aminopyrazolo[1,5-a]pyridin-5-yl)-6-methylpyridin-3-yl)oxy)cyclohexan-1-01 (Intermediate 12) and 2-chloro-5-(difluoromethyl)pyrazine (74 mg, 0.45 mmol) was used instead of 4-chloro-2-ethyl-6-methylpyrimidine. MS (ESI): mass calcd. for C₂₃H₂₂F₂N₆O₃, 468.2. m/z found, 469.2 [M+H]⁺. ¹H NMR (500 MHz, DMSO-d6) δ 10.73 (s, 1H), 8.66-8.60 (m, 2H), 8.52-8.48 (m, 1H), 8.38-8.32 (m, 1H), 7.89-7.84 (m, 1H), 7.35 (s, 1H), 7.07 (dd, J=7.2, 2.0 Hz, 1H), 7.00-6.84 (m, 2H), 5.04 (d, J=4.2 Hz, 1H), 4.20-4.11 (m, 3H), 3.89-3.84 (m, 1H), 3.84-3.74 (m, 2H), 2.47 (s, 3H), 1.98-1.87 (m, 1H), 1.74-1.64 (m, 1H).

### Example 296: 2-[2-(difluoromethoxy)ethyl]-6-[[5-[5-[[(2R,3S)-3-hydroxytetrahydrofuran-2-yl]methoxy]-2-methyl-4-pyridyl]pyrazolo[1,5-a]pyridin-2-yl]amino]-4-methyl-pyridazin-3-one

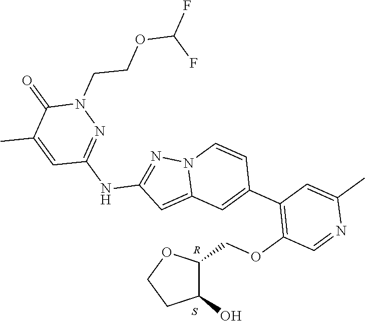

The title compound (52 mg, 32%) was prepared as described in Example 224, except (2R,3S)-2-(((4-(2-aminopyrazolo[1,5-a]pyridin-5-yl)-6-methylpyridin-3-yl)oxy)methyl)tetrahydrofuran-3-ol (Intermediate 25, 102 mg, 0.3 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) and 6-chloro-2-(2-(difluoromethoxy)ethyl)-4-methylpyridazin-3(2H)-one (Example 283, Step A, 79 mg, 0.33 mmol) was used instead of 4-chloro-2-ethyl-6-methylpyrimidine. MS (ESI): mass calcd. for C₂₆H₂₈F₂N605, 542.2. m/z found, 543.2 [M+H]⁺. ¹H NMR (500 MHz, DMSO-d6) δ 9.80 (s, 1H), 8.57-8.53 (m, 1H), 8.35 (s, 1H), 7.85-7.80 (m, 1H), 7.37 (s, 1H), 7.25-7.22 (m, 1H), 7.04 (dd, J=7.2, 2.0 Hz, 1H), 6.86-6.85 (m, 1H), 6.84-6.52 (m, 1H), 5.05 (s, 1H), 4.32-4.24 (m, 4H), 4.20-4.10 (m, 3H), 3.89-3.76 (m, 3H), 2.47 (s, 3H), 2.13-2.09 (m, 3H), 1.99-1.89 (m, 1H), 1.73-1.67 (m, 1H).

### Example 297: (2R,3S)-2-[[6-methyl-4-[2-[(6-methylpyrazin-2-yl)amino]pyrazolo[1,5-a]pyridin-5-yl]-3-pyridyl]oxymethyl]tetrahydrofuran-3-ol

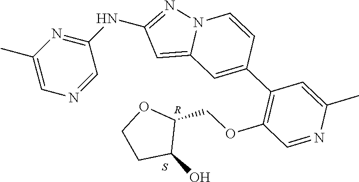

The title compound (71 mg, 55%) was prepared as described in Example 224, except (2R,3S)-2-(((4-(2-aminopyrazolo[1,5-a]pyridin-5-yl)-6-methylpyridin-3-yl)oxy)methyl)tetrahydrofuran-3-ol (Intermediate 25, 102 mg, 0.3 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) and 2-bromo-6-methylpyrazine (57 mg, 0.33 mmol) was used instead of 4-chloro-2-ethyl-6-methylpyrimidine. MS (ESI): mass calcd. for C₂₃H₂₄N₆O₃, 432.2. m/z found, 433.2 [M+H]⁺. ¹H NMR (500 MHz, DMSO-d6) δ 10.16 (s, 1H), 8.61-8.57 (m, 1H), 8.49 (s, 1H), 8.34 (s, 1H), 7.91 (s, 1H), 7.85-7.81 (m, 1H), 7.36 (s, 1H), 7.03 (dd, J=7.2, 2.0 Hz, 1H), 6.92-6.87 (m, 1H), 5.05 (s, 1H), 4.21-4.10 (m, 3H), 3.90-3.74 (m, 3H), 2.47 (s, 3H), 2.42 (s, 3H), 1.99-1.89 (m, 1H), 1.74-1.66 (m, 1H).

### Example 298: (2R,3S)-2-[[4-[2-[(2-tert-butyl-6-methyl-pyrimidin-4-yl)amino]pyrazolo[1,5-a]pyridin-5-yl]-6-methyl-3-pyridyl]oxymethyl]tetrahydrofuran-3-ol

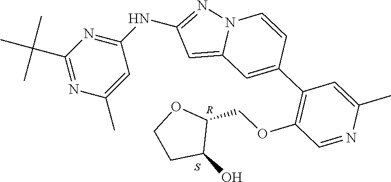

The title compound (66 mg, 45%) was prepared as described in Example 224, except (2R,3S)-2-(((4-(2-aminopyrazolo[1,5-a]pyridin-5-yl)-6-methylpyridin-3-yl)oxy)methyl)tetrahydrofuran-3-ol (Intermediate 25, 102 mg, 0.3 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) and 2-tert-butyl-4-chloro-6-methylpyrimidine (61 mg, 0.33 mmol) was used instead of 4-chloro-2-ethyl-6-methylpyrimidine. MS (ESI): mass calcd. for C₂₇H₃₂N₆O₃, 488.3. m/z found, 489.2 [M+H]⁺. ¹H NMR (500 MHz, DMSO-d6) δ 10.18 (s, 1H), 8.60-8.55 (m, 1H), 8.34 (s, 1H), 7.92-7.87 (m, 1H), 7.40 (s, 1H), 7.05 (dd, J=7.2, 2.0 Hz, 2H), 6.81 (s, 1H), 5.04 (d, J=3.9 Hz, 1H), 4.23-4.09 (m, 3H), 3.89-3.83 (m, 2H), 3.83-3.77 (m, 1H), 2.47 (s, 3H), 2.32 (s, 3H), 2.00-1.91 (m, 1H), 1.74-1.66 (m, 1H), 1.37 (s, 9H).

### Example 299: (2R,3S)-2-[[6-methyl-4-[2-[(1-methylindazol-3-yl)amino]pyrazolo[1,5-a]pyridin-5-yl]-3-pyridyl]oxymethyl]tetrahydrofuran-3-ol

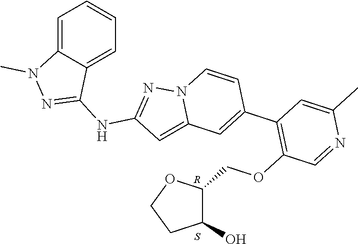

The title compound (93 mg, 66%) was prepared as described in Example 224, except (2R,3S)-2-(((4-(2-aminopyrazolo[1,5-a]pyridin-5-yl)-6-methylpyridin-3-yl)oxy)methyl)tetrahydrofuran-3-ol (Intermediate 25, 102 mg, 0.3 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) and 3-bromo-1-methyl-1h-indazole (70 mg, 0.33 mmol) was used instead of 4-chloro-2-ethyl-6-methylpyrimidine. MS (ESI): mass calcd. for C₂₆H₂₆N₆O₃, 470.2. m/z found, 471.3 [M+H]⁺. ¹H NMR (500 MHz, DMSO-d6) δ 9.86 (s, 1H), 8.55-8.47 (m, 1H), 8.34 (s, 1H), 8.13-8.03 (m, 1H), 7.84-7.77 (m, 1H), 7.49-7.43 (m, 1H), 7.40-7.33 (m, 2H), 7.05-7.00 (m, 1H), 7.00-6.96 (m, 2H), 5.05 (d, J=3.5 Hz, 1H), 4.20-4.07 (m, 3H), 3.93 (s, 3H), 3.90-3.86 (m, 1H), 3.85-3.76 (m, 2H), 2.47 (s, 3H), 2.00-1.90 (m, 1H), 1.75-1.66 (m, 1H).

### Example 300: (2R,3S)-2-[[4-[2-[(1-methylpyrazol-4-yl)amino]pyrazolo[1,5-a]pyridin-5-yl]-6-prop-1-ynyl-3-pyridyl]oxymethyl]tetrahydrofuran-3-ol

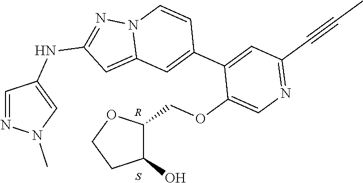

Step A: (2R,3S)-2-(((6-chloro-4-iodopyridin-3-yl)oxy)methyl)tetrahydrofuran-3-ol. To a vial was added 6-chloro-4-iodopyridin-3-ol (929 mg, 3.64 mmol), ((2R,3S)-3-hydroxytetrahydrofuran-2-yl)methyl 4-methylbenzenesulfonate (Intermediate 25, Step A, 825 mg, 3.03 mmol), cesium carbonate (2.37 g, 7.27 mmol), and DMF (8.66 mL, 3.03 mmol). The mixture was heated at 60° C. for 21 h, then worked-up in a separatory funnel using ethyl acetate/water/brine. The organic layer was separated, dried with Na₂SO₄, and purified via FCC (40-100% ethyl acetate in hexanes) to yield the title compound (745 mg, 69%).

Step B: (2R,3S)-2-(((4-(2-aminopyrazolo[1,5-a]pyridin-5-yl)-6-chloropyridin-3-yl)oxy)methyl)tetrahydrofuran-3-ol. The title compound (543 mg, 72%) was prepared as described in Intermediate 25, Step C, except (2R,3S)-2-(((6-chloro-4-iodopyridin-3-yl)oxy)methyl)tetrahydrofuran-3-ol (740 mg, 2.08 mmol) was used instead of (2R,3S)-2-(((4-bromo-6-methylpyridin-3-yl)oxy)methyl)tetrahydrofuran-3-ol.

Step C: (2R,3S)-2-(((4-(2-aminopyrazolo[1,5-a]pyridin-5-yl)-6-(prop-1-yn-1-yl)pyridin-3-yl)oxy)methyl)tetrahydrofuran-3-ol. To a vial was added (2R,3S)-2-(((4-(2-aminopyrazolo[1,5-a]pyridin-5-yl)-6-chloropyridin-3-yl)oxy)methyl)tetrahydrofuran-3-ol (540 mg, 1.50 mmol), 1,1′-bis(diphenylphosphino)ferrocene-palladium(II)dichloride dichloromethane complex (61.8 mg, 0.07 mmol), and 1,4-dioxane (5.99 mL, 1.50 mmol). The vial was capped, then evacuated and backfilled with nitrogen (3×). Tributyl(1-propynyl)tin (0.91 mL, 3.00 mmol) was injected to the vial, and the reaction was heated at 70° C. for 2 h. The reaction mixture was filtered through Celite and concentrated, then purified via FCC (0-100% ethyl acetate (with 10% methanol) in hexanes) to yield the title compound (448 mg, 82%).

Step D: (2R,3S)-2-[[4-[2-[(1-methylpyrazol-4-yl)amino]pyrazolo[1,5-a]pyridin-5-yl]-6-prop-1-ynyl-3-pyridyl]oxymethyl]tetrahydrofuran-3-ol. The title compound (19.8 mg, 16%) was prepared as described in Example 224, except (2R,3S)-2-(((4-(2-aminopyrazolo[1,5-a]pyridin-5-yl)-6-(prop-1-yn-1-yl)pyridin-3-yl)oxy)methyl)tetrahydrofuran-3-ol (100 mg, 0.27 mmol) was used instead of (1r,4r)-4-((4-(2-aminopyrazolo[1,5-a]pyridin-5-yl)-6-methylpyridin-3-yl)oxy)cyclohexan-1-ol (Intermediate 12) and 4-bromo-1-methyl-1h-pyrazole (50 mg, 0.30 mmol) was used instead of 4-chloro-2-ethyl-6-methylpyrimidine. MS (ESI): mass calcd. for C₂₄H₂₄N₆O₃, 444.2. m/z found, 445.1 [M+H]⁺. ¹H NMR (500 MHz, DMSO-d6) δ 8.46-8.43 (m, 2H), 8.41 (s, 1H), 7.78-7.74 (m, 1H), 7.65-7.62 (m, 1H), 7.49 (s, 1H), 7.39-7.35 (m, 1H), 6.85 (dd, J=7.2, 2.0 Hz, 1H), 5.91-5.89 (m, 1H), 5.04 (s, 1H), 4.25-4.14 (m, 3H), 3.89-3.81 (m, 2H), 3.80 (s, 3H), 3.79-3.74 (m, 1H), 2.06 (s, 3H), 1.97-1.87 (m, 1H), 1.72-1.65 (m, 1H).

### Example 301: (2R,3S)-2-[[4-[2-[(1-methylpyrazol-3-yl)amino]pyrazolo[1,5-a]pyridin-5-yl]-6-prop-1-ynyl-3-pyridyl]oxymethyl]tetrahydrofuran-3-ol

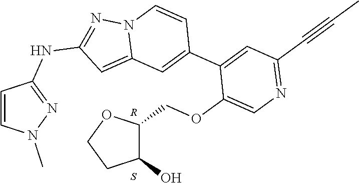

The title compound (11.2 mg, 9%) was prepared as described in Example 300, except 3-bromo-1-methyl-1h-pyrazole (49 mg, 0.30 mmol) was used in Step D instead of 4-bromo-1-methyl-1h-pyrazole. MS (ESI): mass calcd. for C₂₄H₂₄N₆O₃, 444.2. m/z found, 445.2 [M+H]⁺. ¹H NMR (500 MHz, DMSO-d6) δ 9.06 (s, 1H), 8.44 (dd, J=7.2, 0.9 Hz, 1H), 8.41 (s, 1H), 7.74-7.69 (m, 1H), 7.52-7.47 (m, 2H), 6.89 (dd, J=7.2, 2.0 Hz, 1H), 6.44-6.38 (m, 1H), 6.06 (d, J=2.2 Hz, 1H), 5.05 (s, 1H), 4.27-4.13 (m, 3H), 3.89-3.85 (m, 1H), 3.84-3.75 (m, 2H), 3.73 (s, 3H), 2.06 (s, 3H), 1.96-1.88 (m, 1H), 1.72-1.66 (m, 1H).

### Example 302: N-(2,6-dimethylpyrimidin-4-yl)-5-[5-[[(1S,4S)-5-oxa-2-azabicyclo[2.2.1]heptan-4-yl]methoxy]-2-prop-1-ynyl-4-pyridyl]pyrazolo[1,5-a]pyridin-2-amine

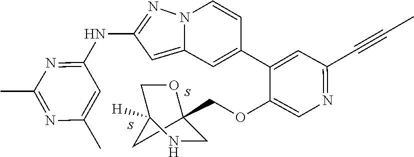

Step A: Tert-butyl (1S,4S)-1-(((4-(2-((2,6-dimethylpyrimidin-4-yl)amino)pyrazolo[1,5-a]pyridin-5-yl)-6-(prop-1-yn-1-yl)pyridin-3-yl)oxy)methyl)-2-oxa-5-azabicyclo[2.2.1]heptane-5-carboxylate. The title compound (100 mg, 83%) was prepared as described in Example 300, step C except tert-butyl (1S,4S)-1-(((6-chloro-4-(2-((2,6-dimethylpyrimidin-4-yl)amino)pyrazolo[1,5-a]pyridin-5-yl)pyridin-3-yl)oxy)methyl)-2-oxa-5-azabicyclo[2.2.1]heptane-5-carboxylate (Example 303, Step B) was used instead of (2R,3S)-2-4(4-(2-aminopyrazolo[1,5-a]pyridin-5-yl)-6-chloropyridin-3-yl)oxy)methyl)tetrahydrofuran-3-ol.

Step B: N-(2,6-dimethylpyrimidin-4-yl)-5-[5-[[(1S,45)-5-oxa-2-azabicyclo[2.2.1]heptan-4-yl]methoxy]-2-prop-1-ynyl-4-pyridyl]pyrazolo[1,5-a]pyridin-2-amine. The title compound (60 mg, 73%) was prepared as described in Example 9, Step B, except tert-butyl (1S,4S)-1-(((4-(2-((2,6-dimethylpyrimidin-4-yl)amino)pyrazolo[1,5-a]pyridin-5-yl)-6-(prop-1-yn-1-yl)pyridin-3-yl)oxy)methyl)-2-oxa-5-azabicyclo[2.2.1]heptane-5-carboxylate was used instead of tert-butyl 4-((6-cyano-4-(2-(cyclopropanecarboxamido)pyrazolo[1,5-a]pyridin-5-yl)pyridin-3-yl)oxy)-4-(trifluoromethyl)piperidine-1-carboxylate. MS (ESI): mass calcd. for C₂₇H₂₇N₇O₂, 481.2. m/z found, 482.3 [M+H]⁺. ¹H NMR (500 MHz, DMSO-d6) δ 10.20 (s, 1H), 8.63-8.55 (m, 1H), 8.46 (s, 1H), 7.94-7.84 (m, 1H), 7.55 (s, 1H), 7.07 (dd, J=7.2, 2.0 Hz, 1H), 7.02 (s, 1H), 6.85 (s, 1H), 4.54 (s, 2H), 3.79 (dd, J=6.9, 1.8 Hz, 1H), 3.69 (d, J=6.9 Hz, 1H), 3.58-3.49 (m, 1H), 2.90 (d, J=9.7 Hz, 1H), 2.86-2.80 (m, 1H), 2.46 (s, 3H), 2.30 (s, 3H), 2.07 (s, 3H), 1.70-1.59 (m, 2H).

### Example 303: 5-[2-chloro-5-[[(1S,4S)-5-oxa-2-azabicyclo[2.2.1]heptan-4-yl]methoxy]-4-pyridyl]-N-(2,6-dimethylpyrimidin-4-yl)pyrazolo[1,5-a]pyridin-2-amine

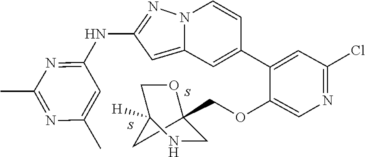

The title compound was prepared using a similar tosic acid displacement method described in Example 188, except Intermediate 26 was used instead of Intermediate 7, and tert-butyl (1S,4S)-1-((tosyloxy)methyl)-2-oxa-5-azabicyclo[2.2.1]heptane-5-carboxylate was used instead of 4,4-difluoro-(1-hydroxycyclohexyl)methyl 4-methylbenzenesulfonate. This was followed by a Boc-deprotection step similar to that described in Example 9, Step B. MS (ESI): mass calcd. for C₂₄H₂₄ClN₇O₂, 477.2. m/z found, 478.2 [M+H]⁺. ¹H NMR (500 MHz, DMSO-d6) δ 10.22 (s, 1H), 8.65-8.57 (m, 1H), 8.35 (s, 1H), 7.96-7.89 (m, 1H), 7.63 (s, 1H), 7.08 (dd, J=7.2, 2.0 Hz, 1H), 7.01 (s, 1H), 6.88 (s, 1H), 4.52 (s, 2H), 3.79 (dd, J=6.9, 1.8 Hz, 1H), 3.69 (d, J=6.9 Hz, 1H), 3.56-3.49 (m, 1H), 2.90 (d, J=9.7 Hz, 1H), 2.85-2.80 (m, 1H), 2.46 (s, 3H), 2.30 (s, 3H), 1.69-1.58 (m, 2H).

### Example 304: (*S)-5-[5-(2-amino-3,3,3-trifluoro-2-methyl-propoxy)-2-methyl-4-pyridyl]-N-(2,6-dimethylpyrimidin-4-yl)pyrazolo[1,5-a]pyridin-2-amine

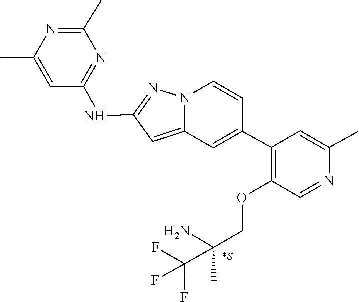

The title compound (152 mg, 45%) was prepared as described in Example 198, except 2-amino-3,3,3-trifluoro-2-methylpropan-1-ol HCl (1.50 equiv.) was used instead of trans-1,4-cyclohexanediol to give racemate which was further purified by SFC over Lux-Cellulose-4 (2×25 cm), (eluent: 40% (v/v) supercritical CO₂ in MeOH with 0.1% DEA). The second elute isomer was designated as *S isomer. MS (ESI): mass calcd. for C₂₃H₂₄F₃N₇O, 471.2. m/z found, 472.2 [M+H]⁺. ¹H NMR (500 MHz, Chloroform-d) δ 8.41 (dt, J=7.3, 0.9 Hz, 1H), 8.27 (s, 1H), 7.62 (dd, J=2.0, 0.9 Hz, 1H), 7.55 (s, 1H), 7.20 (s, 1H), 7.11 (s, 1H), 6.94 (dd, J=7.2, 1.9 Hz, 1H), 6.58 (s, 1H), 4.22 (d, J=9.4 Hz, 1H), 3.98 (dd, J=9.5, 1.3 Hz, 1H), 3.51 (s, OH), 2.61 (d, J=17.2 Hz, 6H), 2.47 (s, 3H), 1.60 (s, 2H), 1.31 (s, 3H).

### Example 305: 2,4-dimethyl-6-((5-(2-methyl-5-(((1s,4s)-4-(methylsulfonyl)cyclohexyl)oxy)pyridin-4-yl)pyrazolo[1,5-a]pyridin-2-yl)amino)pyridazin-3(2H)-one

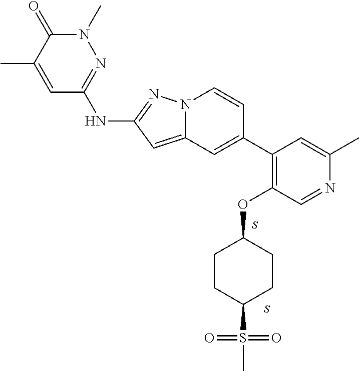

The title compound (80 mg, 22%) was prepared as described in Example 269, except 4-(methyl sulfonyl)cyclohexanol (1.50 equiv.) was used instead of trans-1,4-cyclohexanediol to give 2,4-dimethyl-6-((5-(2-methyl-5-((4-(methylsulfonyl)cyclohexyl)oxy)pyridin-4-yl)pyrazolo[1,5-a]pyridin-2-yl)amino)pyridazin-3(2H)-one which was separated by chiral SFC (AS-H, (2×25 cm), 55% isopropanol/CO₂), followed by SFC (SFC-B (2×25 cm), 25% MeOH/CO₂, 60% CO₂) to afford two peaks. The first elute isomer was designated as *S,S isomer. MS (ESI): mass calcd. for C₂₆H₃₀N₆O₄S, 522.2. m/z found, 523.2 [M+H]⁺. ¹H NMR (500 MHz, Chloroform-d) δ 8.36-8.30 (m, 1H), 8.28 (s, 1H), 7.66 (dd, J=2.0, 0.9 Hz, 1H), 7.21 (s, 1H), 7.19-7.12 (m, 2H), 6.94 (dd, J=7.2, 2.0 Hz, 1H), 6.78 (d, J=0.8 Hz, 1H), 3.79 (s, 3H), 2.93-2.82 (m, 1H), 2.57 (d, J=12.3 Hz, 6H), 2.30-2.23 (m, 3H), 2.06-1.98 (m, 2H), 1.87-1.80 (m, 3H), 1.79 (dd, J=13.0, 3.6 Hz, 1H), 1.62-1.58 (m, 2H).

### Example 306: 2,4-dimethyl-6-((5-(2-methyl-5-(((1r,4r)-4-(methylsulfonyl)cyclohexyl)oxy)pyridin-4-yl)pyrazolo[1,5-a]pyridin-2-yl)amino)pyridazin-3(2H)-one

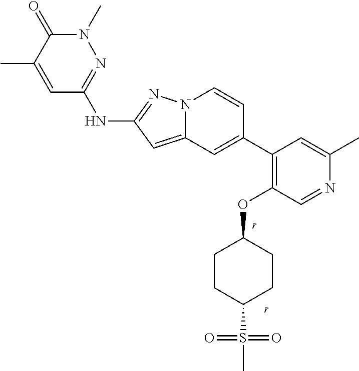

The title compound (64 mg, 18%) was prepared as described in Example 305, except the second elute isomer was designated as *r,r isomer. MS (ESI): mass calcd. for C₂₆H₃₀N₆O₄S, 522.2. m/z found, [M+H]⁺. ¹H NMR (500 MHz, Chloroform-d) δ 8.32-8.27 (m, 2H), 7.62 (dd, J=2.0, 0.9 Hz, 1H), 7.20 (s, 1H), 7.12 (q, J=1.3 Hz, 1H), 6.96-6.88 (m, 2H), 6.72 (d, J=0.8 Hz, 1H), 3.79 (s, 3H), 2.85 (s, 3H), 2.89-2.79 (m, 1H), 2.58 (s, 3H), 2.35-2.23 (m, 6H), 2.24 (d, J=3.7 Hz, 1H), 1.72 (d, J=3.2 Hz, 1H), 1.65 (dd, J=13.3, 3.3 Hz, 1H), 1.52-1.42 (m, 2H).

### Example 307: 2,2-dimethyl-3-[[6-methyl-4-[2-(1H-pyrrolo[2,3-b]pyridin-6-ylamino)pyrazolo[1,5-a]pyridin-5-yl]-3-pyridyl]oxy]propanenitrile

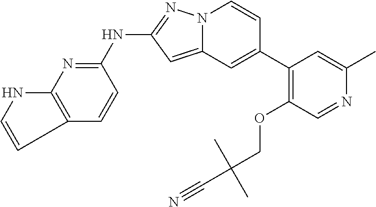 [SMILES: Cc1cc(-c2ccn3nc(Nc4ccc5cc[nH]c5n4)cc3c2)c(OCC(C)(C)C#N)cn1]

Step A: A mixture of consisting of Intermediate 8 (500 mg, 2.06 mmol), 6-bromo-1H-pyrrolo[2,3-b]pyridine (447 mg, 2.27 mmol), tBuBrettphos-Pd-G3 (88 mg, 0.10 mmol), Cs₂CO₃ (1.35 g, 4.13 mmol) and 1,4-dioxane (8.8 mL) was sonicated under vacuum for 2 minutes, then vented with Argon. The mixture was stirred at 80° C. for 3 hours before being cooled to 23° C. The mixture was filtered through celite and concentrated to dryness in vacuo to give the crude product, which was purified by silica gel chromatography (0-10% 2N NH₃ in MeOH/DCM) to give N-(5-(5-fluoro-2-methylpyridin-4-yl)pyrazolo[1,5-a]pyridin-2-yl)-1H-pyrrolo[2,3-b]pyridin-6-amine as a yellow solid (620 mg, 84% yield).

Step B: To a solution of 3-hydroxy-2,2-dimethylpropanenitrile (103 mg, 1.05 mmol) in NMP (2.10 mL) was added NaH (110 mg, 60% dispersion in mineral oil). The mixture allowed to stir at 23° C. for 10 min. To this was N-(5-(5-fluoro-2-methylpyridin-4-yl)pyrazolo[1,5-a]pyridin-2-yl)-1H-pyrrolo[2,3-b]pyridin-6-amine (250 mg, 0.70 mmol), and the reaction was stirred at 80° C. for 16 hours. The reaction was cooled to 23° C. and quenched with MeOH and then concentrated in vacuo. The crude mixture was purified via prep HPLC (Basic ISCO, 20-70% AcN/20 mM aq NH₄OH) and lyophilized to yield the title compound as a white solid (115 mg, 38%). MS (ESI): mass calcd. for C₂₅H₂₃N₇O, 437.2. m/z found, 438.2 [M+H]⁺. ¹H NMR (400 MHz, Chloroform-d) δ 8.38-8.33 (m, 1H), 8.17 (s, 1H), 7.86 (dd, J=8.4, 0.9 Hz, 1H), 7.72 (dt, J=2.0, 0.9 Hz, 1H), 7.32 (s, 1H), 7.26 (s, 1H), 7.12 (dd, J=3.5, 0.9 Hz, 1H), 7.03 (dd, J=8.4, 0.9 Hz, 1H), 6.96-6.91 (m, 1H), 6.86 (d, J=1.0 Hz, 1H), 6.41 (dd, J=3.5, 1.0 Hz, 1H), 4.02 (d, J=0.8 Hz, 2H), 2.58 (d, J=0.8 Hz, 3H), 1.42 (d, J=0.9 Hz, 6H).

### Example 308: 3-[[4-[2-(imidazo[1,2-b]pyridazin-6-ylamino)pyrazolo[1,5-a]pyridin-5-yl]-6-methyl-3-pyridyl]oxy]-2,2-dimethyl-propanenitrile

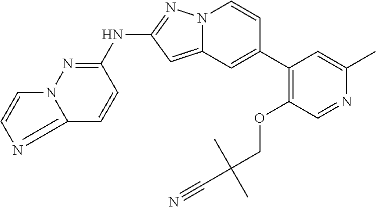 [SMILES: Cc1cc(-c2ccn3nc(Nc4ccc5nccn5n4)cc3c2)c(OCC(C)(C)C#N)cn1]

The title compound (93 mg, 21%) was prepared as described in Example 307, except 6-chloroimidazo[1,2,b]pyridazine was used instead of 6-bromo-1H-pyrrolo[2,3-b]pyridine in Step A. MS (ESI): mass calcd. for C₂₄H₂₂N₈O, 438.2. m/z found, 439.2 [M+H]⁺. ¹H NMR (400 MHz, Chloroform-d) δ 8.36 (dt, J=7.1, 0.9 Hz, 1H), 8.23 (s, 1H), 7.86-7.79 (m, 2H), 7.73 (dd, J=2.0, 1.0 Hz, 1H), 7.63 (d, J=1.1 Hz, 1H), 7.49 (s, 1H), 7.23 (s, 1H), 7.07-6.97 (m, 2H), 6.92 (d, J=9.8 Hz, 1H), 4.01 (s, 2H), 3.49 (d, J=4.6 Hz, 1H), 2.59 (s, 3H), 1.42 (s, 6H), 1.10 (d, J=5.6 Hz, OH).

### Example 309: 2,2-dimethyl-3-[[6-methyl-4-[2-[[6-methyl-2-(methylamino)pyrimidin-4-yl]amino]pyrazolo[1,5-a]pyridin-5-yl]-3-pyridyl]oxy]propanenitrile

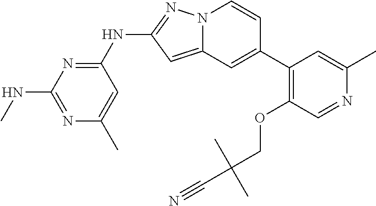

The title compound (210 mg, 41%) was prepared as described in Example 307, except 4-chloro-N,6-dimethylpyrimidin-2-amine was used instead of 6-bromo-1H-pyrrolo[2,3-b]pyridine in Step A. MS (ESI): mass calcd. for C₂₄H₂₆N₈O, 442.2. m/z found, 443.2 [M+H]⁺. ¹H NMR (500 MHz, Chloroform-d) δ 8.38 (dt, J=7.2, 0.9 Hz, 1H), 8.22 (s, 1H), 7.79 (s, 1H), 7.70 (dd, J=1.9, 0.9 Hz, 1H), 7.22 (s, 1H), 6.98 (dd, J=7.2, 2.0 Hz, 1H), 6.80 (s, 1H), 6.32 (s, 1H), 5.18 (d, J=6.0 Hz, 1H), 3.99 (s, 2H), 3.04 (d, J=5.0 Hz, 3H), 2.58 (s, 3H), 2.29 (s, 3H), 1.40 (s, 6H).

### Example 310: 3-[[4-[2-[(2-cyclopropyl-6-methyl-pyrimidin-4-yl)amino]pyrazolo[1,5-a]pyridin-5-yl]-6-methyl-3-pyridyl]oxy]-2,2-dimethyl-propanenitrile

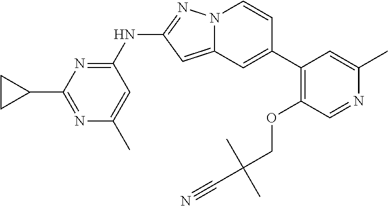 [SMILES: Cc1cc(-c2ccn3nc(Nc4cc(C)nc(C5CC5)n4)cc3c2)c(OCC(C)(C)C#N)cn1]

The title compound (239 mg, 74%) was prepared as described in Example 307, except 4-chloro-2-cyclopropyl-6-methylpyrimidine was used instead of 6-bromo-1H-pyrrolo[2,3-b]pyridine in Step A. MS (ESI): mass calcd. for C₂₆H₂₇N₇O, 453.2. m/z found, 454.2 [M+H]⁺. 1H NMR (500 MHz, Chloroform-d) δ 8.57 (d, J=2.5 Hz, 1H), 8.47-8.41 (m, 1H), 8.24 (s, 1H), 7.72 (dd, J=1.9, 0.9 Hz, 1H), 7.24 (s, 1H), 7.02 (dd, J=7.2, 2.0 Hz, 1H), 6.84 (s, 1H), 6.73 (s, 1H), 4.00 (s, 2H), 2.58 (s, 3H), 2.40 (s, 3H), 2.17-2.11 (m, 1H), 1.41 (s, 6H), 1.18 (dt, J=6.3, 3.2 Hz, 2H), 1.05-0.95 (m, 2H).

### Example 311: (*R)-6-[[5-[5-(2-amino-3,3,3-trifluoro-2-methyl-propoxy)-2-methyl-4-pyridyl]pyrazolo[1,5-a]pyridin-2-yl]amino]-2,4-dimethyl-pyridazin-3-one

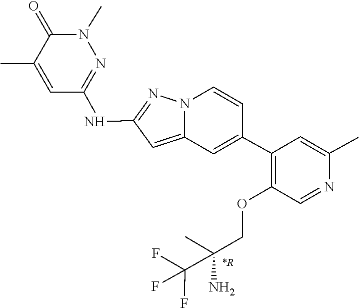

Step A: 6-((5-(5-fluoro-2-methylpyridin-4-yl)pyrazolo[1,5-a]pyridin-2-yl)amino)-2,4-dimethylpyridazin-3(2H)-one. The title compound (1.2 g, 80%) was prepared as described in Example 260 except 5-(5-Fluoro-2-methylpyridin-4-yl)pyrazolo[1,5-a]pyridin-2-amine (Intermediate 8, 1 g, 4.13 mmol) was used instead of 1-((4-(2-aminopyrazolo[1,5-a]pyridin-5-yl)-6-methylpyridin-3-yl)oxy)-2-methylpropan-2-ol and 6-chloro-2,4-dimethylpyridazin-3(2h)-one was used instead of 6-chloroimidazo[1,2-b]pyridazine.

Step B: (*R)-6-[[5-[5-(2-amino-3,3,3-trifluoro-2-methyl-propoxy)-2-methyl-4-pyridyl]pyrazolo[1,5-a]pyridin-2-yl]amino]-2,4-dimethyl-pyridazin-3-one. The title compound (205 mg, 31%) was prepared as described in Example 136 except 6-((5-(5-fluoro-2-methylpyridin-4-yl)pyrazolo[1,5-a]pyridin-2-yl)amino)-2,4-dimethylpyridazin-3(2H)-one (500 mg, 1.37 mmol) was used instead of N-(5-(5-fluoro-2-methylpyridin-4-yl)pyrazolo[1,5-a]pyridin-2-yl)cyclopropanecarboxamide and 2-amino-3,3,3-trifluoro-2-methylpropan-1-ol HCl (370 mg, 2.06 mmol) was used instead of endo-7-hydroxy-3-oxa-9-azabicyclo[3.3.1]nonane hydrochloride. A further purification by chiral SFC (Lux-Cellulose-4 (2×25 cm), 35% ethanol (0.1% DEA)/CO₂, 100 bar, 50 mL/min, 220 nm) was performed and the first isomer to elute was designated as the *R isomer (205 mg, 31%). MS (ESI): mass calcd. for C₂₃H₂₄F₃N₇O₂, 487.2. m/z found, 488.2 [M+H]⁺. ¹H NMR (400 MHz, Chloroform-d) δ 8.32-8.23 (m, 2H), 7.60 (dd, J=1.9, 0.9 Hz, 1H), 7.18 (s, 1H), 7.12 (d, J=1.3 Hz, 1H), 7.06 (s, 1H), 6.90 (dd, J=7.2, 1.9 Hz, 1H), 6.68 (d, J=0.8 Hz, 1H), 4.20 (d, J=9.4 Hz, 1H), 3.95 (dt, J=9.5, 1.3 Hz, 1H), 3.77 (s, 3H), 2.57 (s, 3H), 2.25 (d, J=1.2 Hz, 3H), 1.74 (s, 2H), 1.32-1.20 (m, 3H),

### Example 312: (*S)-6-[[5-[5-(2-amino-3,3,3-trifluoro-2-methyl-propoxy)-2-methyl-4-pyridyl]pyrazolo[1,5-a]pyridin-2-yl]amino]-2,4-dimethyl-pyridazin-3-one

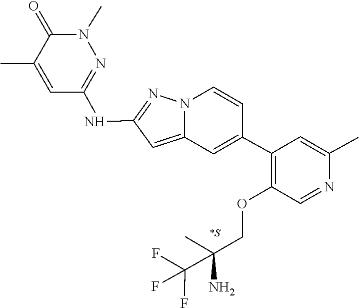

The title compound (210 mg, 31%) was prepared as described in Example 311 except the title compound is the second peak to elute from the chiral SFC and is designated as the *S isomer. MS (ESI): mass calcd. for C₂₃H₂₄F₃N₇O₂, 487.2. m/z found, 488.2 [M+H]⁺. ¹H NMR (400 MHz, Chloroform-d) δ 8.32-8.23 (m, 2H), 7.60 (dd, J=2.0, 0.9 Hz, 1H), 7.18 (s, 1H), 7.15-7.04 (m, 2H), 6.90 (dd, J=7.2, 1.9 Hz, 1H), 6.68 (d, J=0.8 Hz, 1H), 4.20 (d, J=9.5 Hz, 1H), 3.95 (dt, J=9.3, 1.3 Hz, 1H), 3.77 (s, 3H), 2.57 (s, 3H), 2.25 (d, J=1.3 Hz, 3H), 1.75 (s, 2H), 1.32-1.20 (m, 3H).

### Example 313: 1-[[4-[2-[(2,6-dimethylpyrimidin-4-yl)amino]pyrazolo[1,5-a]pyridin-5-yl]-6-methyl-3-pyridyl]oxymethyl]cyclopropanecarboxamide

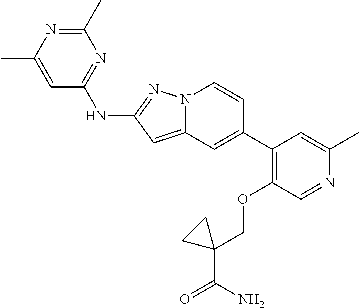

The title compound (236 mg, 74%) was prepared as described in Example 136 except N-(2,6-dimethylpyrimidin-4-yl)-5-(5-fluoro-2-methylpyridin-4-yl)pyrazolo[1,5-a]pyridin-2-amine (Intermediate 15, 250 mg, 0.72 mmol) was used instead of N-(5-(5-fluoro-2-methylpyridin-4-yl)pyrazolo[1,5-a]pyridin-2-yl)cyclopropanecarboxamide and 1-(hydroxymethyl)cyclopropanecarboxamide (124 mg, 1.08 mmol) was used instead of endo-7-hydroxy-3-oxa-9-azabicyclo[3.3.1]nonane hydrochloride. MS (ESI): mass calcd. for C₂₄H₂₅N₇O₂, 443.2. m/z found, 442.2 [M+H]⁺. ¹H NMR (500 MHz, Chloroform-d) δ 9.05 (s, 1H), 8.36-8.30 (m, 1H), 8.26 (s, 1H), 7.60 (dd, J=2.0, 0.9 Hz, 1H), 7.18 (s, 1H), 6.96 (s, 1H), 6.91 (dd, J=7.2, 2.0 Hz, 1H), 6.69 (s, 1H), 6.32 (s, 1H), 4.14 (s, 2H), 2.70 (s, 1H), 2.55 (d, J=9.6 Hz, 6H), 2.38 (s, 3H), 1.37-1.31 (m, 2H), 0.82 (q, J=4.4 Hz, 2H).

### Example 314: 5-[5-(3-aminocyclobutoxy)-2-methyl-4-pyridyl]-N-(2,6-dimethylpyrimidin-4-yl)pyrazolo[1,5-a]pyridin-2-amine

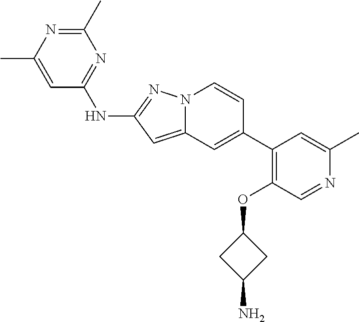

The title compound (235 mg, 79%) was prepared as described in Example 136 except N-(2,6-dimethylpyrimidin-4-yl)-5-(5-fluoro-2-methylpyridin-4-yl)pyrazolo[1,5-a]pyridin-2-amine (Intermediate 15, 250 mg, 0.72 mmol) was used instead of N-(5-(5-fluoro-2-methylpyridin-4-yl)pyrazolo[1,5-a]pyridin-2-yl)cyclopropanecarboxamide and cis-3-aminocyclobutanoyl hydrochloride (133 mg, 1.08 mmol) was used instead of endo-7-hydroxy-3-oxa-9-azabicyclo[3.3.1]nonane hydrochloride. MS (ESI): mass calcd. for C₂₃H₂₅N₇O, 415.2. m/z found, 416.2 [M+H]⁺. ¹H NMR (500 MHz, Chloroform-d) δ 8.55 (s, 1H), 8.47 (dt, J=7.2, 0.9 Hz, 1H), 8.12 (s, 1H), 7.65 (dd, J=2.0, 0.9 Hz, 1H), 7.18 (s, 1H), 7.03 (s, 1H), 6.96 (dd, J=7.2, 1.9 Hz, 1H), 6.63 (s, 1H), 4.40 (p, J=7.0 Hz, 1H), 3.47 (s, 2H), 3.24-3.15 (m, 1H), 2.93-2.86 (m, 2H), 2.62 (s, 3H), 2.55 (s, 3H), 2.44 (s, 3H), 1.90-1.83 (m, 2H).

### Example 315: 4-[[4-[2-[(2,6-dimethylpyrimidin-4-yl)amino]pyrazolo[1,5-a]pyridin-5-yl]-6-methyl-3-pyridyl]oxy]-2,2-dimethyl-butanenitrile

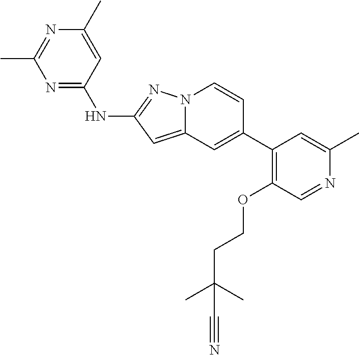

The title compound (12 mg, 4%) was prepared as described in Example 136 except N-(2,6-dimethylpyrimidin-4-yl)-5-(5-fluoro-2-methylpyridin-4-yl)pyrazolo[1,5-a]pyridin-2-amine (Intermediate 15, 250 mg, 0.72 mmol) was used instead of N-(5-(5-fluoro-2-methylpyridin-4-yl)pyrazolo[1,5-a]pyridin-2-yl)cyclopropanecarboxamide and 4-hydroxy-2,2-dimethylbutanenitrile (122 mg, 1.08 mmol) was used instead of endo-7-hydroxy-3-oxa-9-azabicyclo[3.3.1]nonane hydrochloride. MS (ESI): mass calcd. for C₂₅H₂₇N₇O, 441.2. m/z found, 442.2 [M+H]⁺. ¹H NMR (500 MHz, Chloroform-d) δ 8.41-8.38 (m, 1H), 8.29 (s, 1H), 7.69-7.58 (m, 2H), 7.17 (d, J=6.8 Hz, 1H), 7.07 (s, 1H), 6.91 (dd, J=7.2, 1.9 Hz, 1H), 6.59 (s, 1H), 4.32-4.20 (m, 2H), 2.59 (dd, J=20.7, 2.1 Hz, 6H), 2.47-2.43 (m, 3H), 2.02 (t, J=6.4 Hz, 2H), 1.37 (s, 5H), 1.30 (s, 1H).

### Example 316: 3-[[4-[2-[(1-cyclopropyl-5-methyl-6-oxo-pyridazin-3-yl)amino]pyrazolo[1,5-a]pyridin-5-yl]-6-methyl-3-pyridyl]oxy]-2,2-dimethyl-propanenitrile

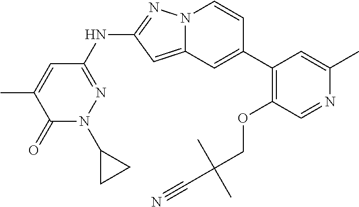

Step A: 6-chloro-2-cyclopropyl-4-methylpyridazin-3(2H)-one. To a vial was added 6-chloro-4-methylpyridazin-3(2h)-one (1 g, 6.92 mmol), cesium carbonate (5.64 g, 17.3 mmol), DMF (34.6 mL, 6.92 mmol), and iodocyclopropane (1.57 g, 9.34 mmol). The reaction mixture was stirred at 140° C. for 16 hr and worked-up in a separatory funnel with EtOAc/water/brine. The extracted organics were dried with Na₂SO₄ and purified via FCC (0-50% EtOAc/hexanes) to afford the title product as a yellow oil (320 mg, 25%).

Step B: 2-cyclopropyl-6-((5-(5-fluoro-2-methylpyridin-4-yl)pyrazolo[1,5-a]pyridin-2-yl)amino)-4-methylpyridazin-3(2H)-one. The title compound (700 mg, 87%) was prepared as described in Example 224 except 5-(5-Fluoro-2-methylpyridin-4-yl)pyrazolo[1,5-a]pyridin-2-amine (Intermediate 8, 501 mg, 2.07 mmol) was used instead of (1r,4r)-4-((4-(2-aminopyrazolo[1,5-a]pyridin-5-yl)-6-methylpyridin-3-yl)oxy)cyclohexan-1-ol and 6-chloro-2-cyclopropyl-4-methylpyridazin-3(2H)-one (420 mg, 2.28 mmol) was used instead of 4-chloro-2-ethyl-6-methylpyrimidine.

Step C: 3-[[4-[2-[(1-cyclopropyl-5-methyl-6-oxo-pyridazin-3-yl)amino]pyrazolo[1,5-a]pyridin-5-yl]-6-methyl-3-pyridyl]oxy]-2,2-dimethyl-propanenitrile. The title compound (210 mg, 70%) was prepared as described in Example 136 except 2-cyclopropyl-6-((5-(5-fluoro-2-methylpyridin-4-yl)pyrazolo[1,5-a]pyridin-2-yl)amino)-4-methylpyridazin-3(2H)-one (250 mg, 0.64 mmol) was used instead of N-(5-(5-fluoro-2-methylpyridin-4-yl)pyrazolo[1,5-a]pyridin-2-yl)cyclopropanecarboxamide and 3-hydroxy-2,2-dimethylpropanenitrile (95 mg, 0.96 mmol) was used instead of endo-7-hydroxy-3-oxa-9-azabicyclo[3.3.1]nonane hydrochloride. MS (ESI): mass calcd. for C₂₆H₂₇N₇O₂, 469.2. m/z found, 470.2 [M+H]⁺. ¹H NMR (500 MHz, Chloroform-d) δ 8.52-8.45 (m, 1H), 8.33 (d, J=7.2 Hz, 1H), 8.24 (s, 1H), 7.76 (d, J=2.2 Hz, 1H), 7.26 (s, 1H), 7.08 (q, J=1.3 Hz, 1H), 7.01 (dd, J=7.2, 2.0 Hz, 1H), 6.77 (d, J=0.8 Hz, 1H), 4.02 (s, 2H), 3.04 (d, J=15.3 Hz, 1H), 2.58 (s, 3H), 2.25-2.21 (m, 3H), 1.42 (s, 6H), 1.29-1.22 (m, 2H), 1.06-0.96 (m, 2H).

### Example 317: 2-cyclopropyl-6-[[5-[5-[[(2R,3S)-3-hydroxytetrahydrofuran-2-yl]methoxy]-2-methyl-4-pyridyl]pyrazolo[1,5-a]pyridin-2-yl]amino]-4-methyl-pyridazin-3-one

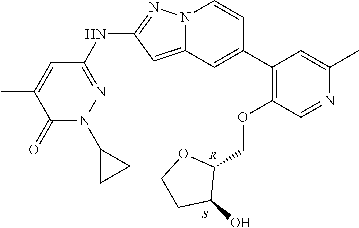

The title compound (97 mg, 78%) was prepared as described in Example 316 except in Step C, (2R,3S)-2-(hydroxymethyl)tetrahydrofuran-3-ol (61 mg, 0.51 mmol) was used instead of 3-hydroxy-2,2-dimethylpropanenitrile. MS (ESI): mass calcd. for C₂₆H₂₈N₆O₄, 488.2. m/z found, 489.3 [M+H]⁺. ¹H NMR (500 MHz, Chloroform-d) δ 8.27-8.18 (m, 2H), 7.86 (s, 1H), 7.69 (dd, J=1.9, 0.9 Hz, 1H), 7.16 (s, 1H), 6.94-6.86 (m, 2H), 6.62 (d, J=0.8 Hz, 1H), 4.41-4.35 (m, 1H), 4.23-4.10 (m, 4H), 4.02 (dd, J=8.4, 5.4 Hz, 2H), 3.81 (s, 1H), 2.53 (s, 3H), 2.15-2.04 (m, 4H), 1.96-1.88 (m, 1H), 1.23-1.14 (m, 2H), 1.02-0.90 (m, 2H).

### Example 318: 2-cyclopropyl-6-[[5-[5-(4-hydroxycyclohexoxy)-2-methyl-4-pyridyl]pyrazolo[1,5-a]pyridin-2-yl]amino]-4-methyl-pyridazin-3-one

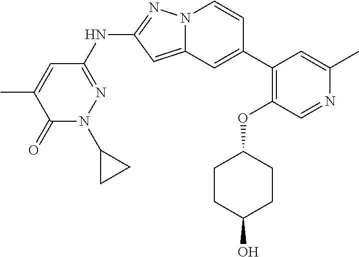

The title compound (63 mg, 51%) was prepared as described in Example 316 except in Step C, trans-1,4-cyclohexanediol (60 mg, 0.51 mmol) was used instead of 3-hydroxy-2,2-dimethylpropanenitrile. MS (ESI): mass calcd. for C₂₇H₃₀N₆O₃, 486.2. m/z found, 487.2 [M+H]⁺. ¹H NMR (500 MHz, Chloroform-d) δ 8.30-8.23 (m, 2H), 7.63 (dd, J=1.9, 0.9 Hz, 1H), 7.49 (s, 1H), 7.19 (s, 1H), 7.03 (q, J=1.2 Hz, 1H), 6.96 (dd, J=7.2, 1.9 Hz, 1H), 6.65 (d, J=0.9 Hz, 1H), 4.30-4.24 (m, 1H), 4.23-4.18 (m, 1H), 3.75 (q, J=8.8, 6.1 Hz, 1H), 2.55 (s, 3H), 2.24 (d, J=1.3 Hz, 3H), 2.15-2.10 (m, 1H), 2.11-2.02 (m, 1H), 2.06 (s, 2H), 1.96-1.86 (m, 1H), 1.59-1.48 (m, 2H), 1.43-1.34 (m, 2H), 1.22 (qt, J=4.6, 2.5 Hz, 2H), 1.06-0.95 (m, 2H).

### Example 319: N-[5-[2-methyl-5-[[(1S,5R)-9-methyl-3-oxa-9-azabicyclo[3.3.1]nonan-7-yl]oxy]-4-pyridyl]pyrazolo[1,5-c]pyrimidin-2-yl]cyclopropanecarboxamide

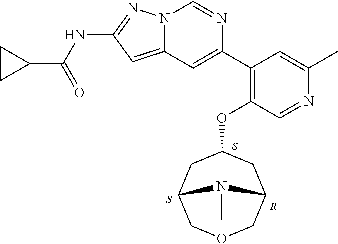

Step A. N-(5-chloropyrazolo[1,5-c]pyrimidin-2-yl)cyclopropanecarboxamide. To a solution of 5-chloropyrazolo[1,5-c]pyrimidin-2-amine and dichloromethane (5.699 mL, 1.326 g/mL, 88.976 mmol) and triethylamine (0.371 mL, 0.728 g/mL, 2.669 mmol) was added cyclopropanecarbonyl chloride (204.625 mg, 1.957 mmol). The reaction was stirred at room temperature for 3 h. LCMS showed the reaction was converted. The crude material was concentrated down under vacuo in the presence of the silica gel. The crude was FCC using 10-100% EtOAc:Hex. The pure fraction was collected and concentrated down to afford the said compound (174 mg, 84%). ¹H NMR (600 MHz, Chloroform-d) δ 8.87 (t, J=1.1 Hz, 1H), 8.19 (s, 1H), 7.35 (d, J=1.3 Hz, 1H), 6.99 (s, 1H), 1.58 (m, 1h), 1.18-1.12 (m, 2H), 1.00-0.90 (m, 2H).

Step B. N-(5-(5-fluoro-2-methylpyridin-4-yl)pyrazolo[1,5-c]pyrimidin-2-yl)cyclopropanecarboxamide. A mixture consisting of N-(5-chloropyrazolo[1,5-c]pyrimidin-2-yl)cyclopropanecarboxamide (174 mg, 0.735 mmol), 5-fluoro-2-methyl-4-(4,4,5,5-tetramethyl-1,3,2-dioxaborolan-2-yl)pyridine (0.209 g, 0.882 mmol), Ruphos-Pd-G2 (0.0571 g, 0.0735 mmol), 1,4-dioxane (5.644 mL, 1.033 g/mL, 66.171 mmol), and aq. K₃PO₄ (1.47 mL, 0.5 M, 0.735 mmol) was stirred at 90° C. for 1 h. LCMS showed the reaction was complete. The crude reaction mixture was diluted with EtOAc and H₂O. The organic layer was separated and washed with H₂O 2×. The organic layer was collected, dried over anhydrous MgSO₄, filtered, and concentrated down in the presence of silica gel and under vacuo. The crude was purified by silica gel chromatography using 20-100% EtOAc:Hex. The pure fractions were concentrated down to afford the title compound (168 mg, 73%). MS (ESI): mass calcd. for C₁₆H₁₄FN₅O, 311.12. m/z found, 312.1 [M+H]+. 1H NMR (600 MHz, DMSO-d6) δ 11.36 (s, 1H), 9.50 (t, J=1.2 Hz, 1H), 8.56 (d, J=3.0 Hz, 1H), 8.16 (d, J=1.4 Hz, 1H), 7.93 (d, J=6.5 Hz, 1H), 7.10 (s, 1H), 2.55 (s, 3H), 1.96 (s, 1H), 0.88-0.81 (m, 4H).

Step C. N-(5-(5-(((1R,5S,7s)-3-oxa-9-azabicyclo[3.3.1]nonan-7-yl)oxy)-2-methylpyridin-4-yl)pyrazolo[1,5-c]pyrimidin-2-yl)cyclopropanecarboxamide. To a 25 mL round bottom flask was added endo-tert-butyl (1R,5S,7s)-7-hydroxy-3-oxa-9-azabicyclo[3.3.1]nonane-9-carboxylate (117.229 mg, 0.482 mmol) and DMF (0.622 mL, 0.944 g/mL, 8.03 mmol) and then NaH (60% dispersion in mineral oil) (32.118 mg, 0.803 mmol). The mixture was stirred for 10 min before syringe into a flask containing N-(5-(5-fluoro-2-methylpyridin-4-yl)pyrazolo[1,5-c]pyrimidin-2-yl)cyclopropanecarboxamide (50 mg, 0.161 mmol) and DMF (0.622 mL, 0.944 g/mL, 8.03 mmol). The reaction was stirred at 80 C in heating block overnight. The crude reaction mixture was quenched with H₂O. It was then diluted with EtOAc and washed with H₂O 4×. The organics were collected, dried over anh. MgSO₄, filtered, and concentrated down under vacuo. The crude was purified via 50-100% EtOAc:Hex and then 0-10% 10% 2M NH₃-MeOH/DCM:EtOAc. To the purified material was added 1:4 TFA:DCM and stirred for 1 h. The crude was concentrated down to a white solid. This material was used in the next step without further purifications.

Step D. N-(5-(2-methyl-5-(((1R,5S,7s)-9-methyl-3-oxa-9-azabicyclo[3.3.1]nonan-7-yl)oxy)pyridin-4-yl)pyrazolo[1,5-c]pyrimidin-2-yl)cyclopropanecarboxamide. To a mixture of N-(5-(5-(((1R,5S,7s)-3-oxa-9-azabicyclo[3.3.1]nonan-7-yl)oxy)-2-methylpyridin-4-yl)pyrazolo[1,5-c]pyrimidin-2-yl)cyclopropanecarboxamide (39.089 mg, 0.09 mmol) and MeOH (0.364 mL, 0.791 g/mL, 8.996 mmol) was added H(CHO)ₙ (0.00648 g, 0.072 mmol) and NaCNBH₃ (0.017 g, 0.27 mmol) in the same time. The mixture was stirred overnight. The reaction was complete by LCMS. The crude material was dissolved with MeOH and purified via ISCO prep. HPLC using 10-60% ACN-H₂O with 20 mM of aq. NH₄OH as modifier. The fractions contained desired material was collected, froze, and lyophilized to afford a white powder (39.4 mg, 56%). MS (ESI): mass calcd. for C₂₄H₂₈N₆O₃, 448.2. m/z found, 449.3 [M+H]⁺. ¹H NMR (500 MHz, Methanol-d4) δ 9.19 (t, J=1.2 Hz, 1H), 8.97 (d, J=1.5 Hz, 1H), 8.15 (s, 1H), 8.00 (s, 1H), 6.95 (s, 1H), 4.00 (d, J=10.9 Hz, 2H), 3.75 (d, J=11.2 Hz, 2H), 2.75 (d, J=6.3 Hz, 2H), 2.63-2.47 (m, 8H), 1.88 (d, J=15.5 Hz, 3H), 1.02 (p, J=4.0 Hz, 2H), 0.92 (dq, J=7.4, 3.8 Hz, 2H).

### Example 320: 6-[2-methyl-5-[[(1S,5R)-3-oxa-9-azabicyclo[3.3.1]nonan-7-yl]oxy]-4-pyridyl]-N-(5-methylpyrazin-2-yl)imidazo[1,2-a]pyrazin-2-amine

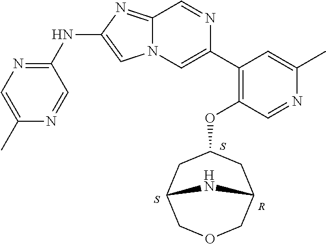

Step A. 6-(5-fluoro-2-methylpyridin-4-yl)imidazo[1,2-a]pyrazin-2-amine. A mixture consisting of 6-bromoimidazo[1,2-a]pyrazin-2-amine (1000 mg, 4.694 mmol), Ruphos-Pd-G2 (0.365 g, 0.469 mmol), and 5-fluoro-2-methyl-4-(4,4,5,5-tetramethyl-1,3,2-dioxaborolan-2-yl)pyridine (1.335 g, 5.633 mmol) was added 1,4-dioxane (36.033 mL, 1.033 g/mL, 422.463 mmol) and aq. K₃PO₄ (9.388 mL, 0.5 M, 4.694 mmol) was stirred at 90° C. for 3 h. The crude was diluted with 20% iPrOH-DCM and the H₂O. The layers were separated. The organics were kept and concentrated under vacuo to a solid. The solid was dissolve with 50 mL of iPrOH/DCM and concentrated down under vacuo in the presence of silica gel. The crude was flashed with 0-10% 2M NH₃ in MeOH-DCM. The pure fractions were concentrated down under vacuo to afford the title compound (640 mg, 56%). MS (ESI): mass calcd. for C₁₂H₁₀FN₅, 243.2. m/z found, 244.1 [M+H]⁺; ¹H NMR (500 MHz, Methanol-d4) δ 8.91 (d, J=1.4 Hz, 1H), 8.70 (t, J=1.0 Hz, 1H), 8.42 (d, J=3.4 Hz, 1H), 8.04 (d, J=6.5 Hz, 1H), 7.36 (d, J=0.7 Hz, 1H), 2.60 (d, J=1.0 Hz, 3H).

Step B. 6-(5-fluoro-2-methylpyridin-4-yl)-N-(5-methylpyrazin-2-yl)imidazo[1,2-a]pyrazin-2-amine. To a mixture of 6-(2-fluoro-5-methylphenyl)imidazo[1,2-a]pyrazin-2-amine from Step A (100 mg, 0.411 mmol), 2-chloro-5-methylpyrazine (58.138 mg, 0.452 mmol), Cs₂CO₃ (0.403 g, 1.238 mmol), TBuBrettPhos Pd G3 (17.563 mg, 0.0206 mmol) was added 1,4-dioxane (0.822 mL, 0.5 M, 0.411 mmol). The mixture was vacuum and back-filled with N₂ 4×. Then, the mixture was heated to 90° C. for 1 h. LCMS showed the reaction was mostly complete. The crude was diluted with DCM and washed with H₂O 2×. The organics were collected, dried over anh. MgSO₄, and concentrated down under vacuo. The material was flashed with 0-10% 2M NH₃ in MeOH/DCM-DCM. The pure fractions were collected then concentrated down to yield the title compound (87 mg, 63%). MS (ESI): mass calcd. for C₁₇H₁₄FN₇, 335.34. m/z found, 336.1 [M+H]⁺; ¹H NMP (500 MHz, DMSO-d6) δ 10.43 (s, 1H), 9.28 (d, J=1.4 Hz, 1H), 8.99 (dd, J=1.4, 0.7 Hz, 1H), 8.59-8.51 (m, 2H), 8.42 (d, J=1.5 Hz, 1H), 8.15 (dd, J=1.5, 0.8 Hz, 1H), 7.98 (d, J=6.5 Hz, 1H), 5.75 (s, 1h), 2.56-2.53 (m, 3H), 2.40 (d, J=0.7 Hz, 3H).

Step C. 6-(5-(((1R,5S,7s)-3-oxa-9-azabicyclo[3.3.1]nonan-7-yl)oxy)-2-methylpyridin-4-yl)-N-(5-methylpyrazin-2-yl)imidazo[1,2-a]pyrazin-2-amine. To a microwave vial was added 6-(5-fluoro-2-methylpyridin-4-yl)-N-(5-methylpyrazin-2-yl)imidazo[1,2-a]pyrazin-2-amine (40 mg, 0.119 mmol), endo-tert-butyl (1R,5S,7s)-7-hydroxy-3-oxa-9-azabicyclo[3.3.1]nonane-9-carboxylate (87.064 mg, 0.358 mmol), sodium tertbutoxide (34 mg, 0.358 mmol) and DMA (0.552 mL, 0.941 g/mL, 5.964 mmol). The reaction was heated to 120° C. for 1 h in microwave. LCMS showed ˜60% desired product. The reaction was heated to 120° C. for 1 h more. LCMS looked very similar. The reaction was heated to 130° C. for 12 h more. The crude reaction mixture was diluted with 10% MeOH/DCM. The crude was flashed with 0-10% MeOH-DCM. The SM coeluted with the product. The mixed fractions were collected and concentrated down under vacuo. The crude was dissolved with DMSO and a little HCl. The crude was purified via preparative HPLC using 0-80% ACN-H₂O with 20 mM of aq. NH₄OH as modifier. The pure fractions were collected and lyophilize down to afford a white powder (11 mg, 21%). MS (ESI): mass calcd. for C₂₄H₂₆N₈O₂, 458.2. m/z found, 459.2 [M+H]⁺. ¹H NMR (500 MHz, Methanol-d4) δ 9.53 (s, 1H), 8.77 (t, J=1.0 Hz, 1H), 8.27 (d, J=0.7 Hz, 1H), 8.22 (d, J=1.5 Hz, 1H), 8.12 (s, 1H), 8.10 (d, J=1.2 Hz, 1H), 7.87 (s, 1H), 4.88 (s, 1H), 3.97-3.87 (m, 4H), 3.37 (s, 3H), 2.46 (s, 5H), 2.36 (s, 3H), 2.26 (d, J=16.2 Hz, 2H).

### Example 321: 6-[2-methyl-5-[[(1S,5R)-3-oxa-9-azabicyclo[3.3.1]nonan-7-yl]oxy]-4-pyridyl]-N-(6-methylpyrazin-2-yl)imidazo[1,2-a]pyrazin-2-amine

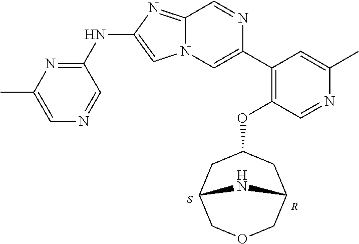

The title compound was prepared in the similar manner of Example 320 except 2-chloro-6-methylpyrazine was used instead of 2-chloro-5-methylpyrazine in Step B (107.5, 39.5%). MS (ESI): mass calcd. for C₂₄H₂₆N₈O₂, 458.2. m/z found, 459.2 [M+H]⁺. ¹H NMR (400 MHz, Methanol-d4) δ 9.95 (d, J=1.4 Hz, 1H), 8.77 (d, J=1.2 Hz, 1H), 8.42 (d, J=0.7 Hz, 1H), 8.09 (d, J=2.9 Hz, 2H), 7.94 (s, 1H), 7.80 (s, 1H), 4.87 (s, 1H), 3.95 (d, J=10.7 Hz, 2H), 3.88 (d, J=11.4 Hz, 2H), 2.95 (d, J=5.9 Hz, 2H), 2.46 (d, J=4.6 Hz, 6H), 2.39 (dd, J=15.1, 6.7 Hz, 2H), 2.06 (d, J=15.6 Hz, 2H).

### Example 322: 6-[2-methyl-5-[[(1S,5R)-3-oxa-9-azabicyclo[3.3.1]nonan-7-yl]oxy]-4-pyridyl]-N-(1-methylpyrazol-4-yl)imidazo[1,2-a]pyrazin-2-amine

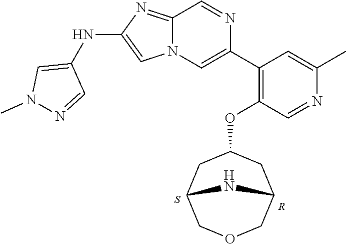

The title compound was prepared in the similar manner of Example 320 except 4-bromo-1-methyl-1H-pyrazole was used instead of 6-(5-fluoro-2-methylpyridin-4-yl)-N-(5-methylpyrazin-2-yl)imidazo[1,2-a]pyrazin-2-amine in step C (54 mg, 13%). MS (ESI): mass calcd. for C₂₃H₂₆N₈O₂, 446.2. m/z found, 447.2 [M+H]⁺. ¹H NMR (400 MHz, DMSO-d6) δ 9.52 (d, J=1.4 Hz, 1H), 8.71 (d, J=1.2 Hz, 1H), 8.56 (s, 1H), 8.21 (s, 1H), 7.85 (s, 1H), 7.73 (s, 1H), 7.34 (s, 1H), 7.26 (s, 1H), 4.81 (tt, J=6.3, 3.7 Hz, 1H), 3.75 (s, 3H), 3.69-3.54 (m, 4H), 2.86 (d, J=7.0 Hz, 2H), 2.39 (s, 3H), 2.26 (dt, J=14.0, 6.8 Hz, 2H), 1.82 (dt, J=14.8, 2.8 Hz, 2H).

### Example 323: N-cyclopropyl-4-[[5-[5-(4-hydroxycyclohexoxy)-2-methyl-4-pyridyl]pyrazolo[1,5-a]pyridin-2-yl]amino]benzenesulfonamide

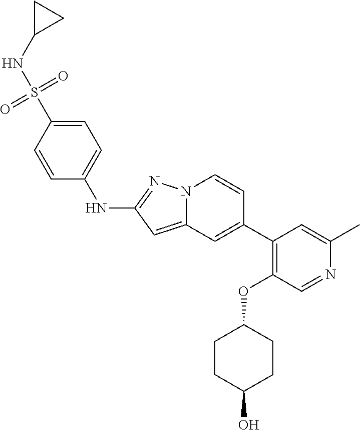 [SMILES: Cc1cc(-c2ccn3nc(Nc4ccc(S(=O)(=O)NC5CC5)cc4)cc3c2)c(O[C@H]2CC[C@H](O)CC2)cn1]

The title compound was prepared as described in Example 239 where N-Cyclopropyl 4-bromobenzenesulfonamide was used instead of 6-bromo-1H-pyrrolo[2,3-b]pyridine (39.6 mg, 24%). MS (ESI): mass calcd. for C₂₈H₃₁N₅O₄S, 533.2. m/z found, [M+H]⁺. ¹H NMR (600 MHz, DMSO-d6) δ 9.58 (s, 1H), 8.59 (dt, J=7.1, 0.9 Hz, 1H), 8.36 (s, 1H), 7.74-7.64 (m, 4H), 7.61 (s, 1H), 6.96 (dd, J=7.2, 2.0 Hz, 1H), 6.24 (d, J=0.9 Hz, 1H), 4.51 (d, J=3.9 Hz, 1H), 4.41 (dt, J=8.8, 4.7 Hz, 1H), 3.50 (s, 1H), 2.13-2.06 (m, 1H), 2.00-1.94 (m, 2H), 1.77-1.70 (m, 2H), 1.46-1.37 (m, 2H), 1.33-1.22 (m, 2H), 0.47 (td, J=7.0, 4.7 Hz, 2H), 0.40-0.35 (m, 2H).

### Example 324: 4-[[5-[5-(4-hydroxycyclohexoxy)-2-methyl-4-pyridyl]pyrazolo[1,5-a]pyridin-2-yl]amino]-N,N-dimethyl-benzenesulfonamide

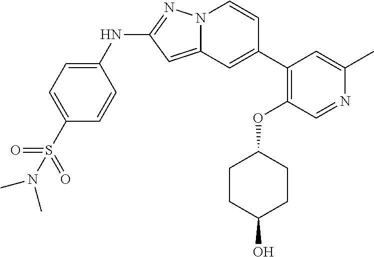

The title compound was prepared as described in Example 239 where 4-bromo-N,N-dimethylbenzenesulfonamide was used instead of 6-bromo-1H-pyrrolo[2,3-b]pyridine (37 mg, 21%). MS (ESI): mass calcd. for C₂₇H₃₁N504S, 521.2. m/z found, 522.3 [M+H]⁺. ¹H NMR (500 MHz, DMSO-d6) δ 9.66 (s, 1H), 8.59 (d, J=7.2 Hz, 1H), 8.36 (s, 1H), 7.75-7.66 (m, 3H), 7.63 (d, J=8.8 Hz, 2H), 7.34 (s, 1H), 6.96 (dd, J=7.2, 2.0 Hz, 1H), 6.25 (s, 1H), 4.51 (d, J=3.8 Hz, 1H), 4.41 (dt, J=9.1, 4.9 Hz, 1H), 2.58 (s, 6H), 2.46 (s, 3H), 1.98 (d, J=12.6 Hz, 2H), 1.73 (t, J=8.4 Hz, 2H), 1.47-1.36 (m, 2H), 1.28 (q, J=12.6, 11.4 Hz, 2H).

### Example 325: (rac)-4-[[4-[2-[(2,6-dimethylpyrimidin-4-yl)amino]pyrazolo[1,5-a]pyridin-5-yl]-6-methyl-3-pyridyl]oxymethyl]-1,5,5-trimethyl-pyrrolidin-2-one

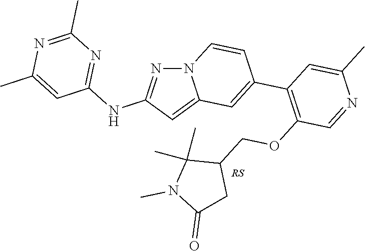

The title compound was prepared by the similar method of Example 1 using 4-(hydroxymethyl)-1,5,5-trimethylpyrrolidin-2-one instead of (R)-(1-methylpyrrolidin-3-yl)methanol and Intermediate 15 instead of N-(5-(5-chloro-2-cyanopyridin-4-yl)pyrazolo[1,5-a]pyridin-2-yl)cyclopropanecarboxamide (15 mg, 36%). MS (ESI): mass calcd. for C₂₇H₃₁N702, 485.3. m/z found, 486.3 [M+H]⁺. ¹H NMR (500 MHz, DMSO-d6) δ 10.12 (s, 1H), 8.55-8.45 (m, 1H), 8.32 (s, 1H), 7.70 (dd, J=2.0, 0.9 Hz, 1H), 7.27 (s, 1H), 6.96 (s, 1H), 6.89 (dd, J=7.2, 2.0 Hz, 1H), 6.80-6.73 (m, 1H), 4.21 (dd, J=9.7, 7.2 Hz, 1H), 4.10 (dd, J=9.7, 6.6 Hz, 1H), 2.48 (s, 3H), 2.41-2.38 (m, 7H), 2.26-2.21 (m, 4H), 2.13 (dd, J=16.5, 10.4 Hz, 1H), 1.09 (s, 3H), 0.94 (s, 3H).

### Example 326: N-cyclopropyl-4-[[5-[5-(2-hydroxy-2-methyl-propoxy)-2-methyl-4-pyridyl]pyrazolo[1,5-a]pyridin-2-yl]amino]-2,6-dimethyl-benzamide

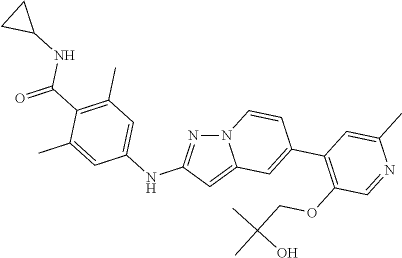 [SMILES: Cc1cc(-c2ccn3nc(Nc4cc(C)c(C(=O)NC5CC5)c(C)c4)cc3c2)c(OCC(C)(C)O)cn1]

The title compound was prepared as described in Example 239 where 4-bromo-N-cyclopropyl-2,6-dimethylbenzamide was used instead of 6-bromo-1H-pyrrolo[2,3-b]pyridine (67 mg, 36%). MS (ESI): mass calcd. for C₂₉H₃₃N₅O₃, 499.3. m/z found, [M+H]⁺. ¹H NMR (600 MHz, DMSO-d6) δ 8.88 (s, 1H), 8.54 (dt, J=7.1, 0.9 Hz, 1H), 8.30 (s, 1H), 8.18 (d, J=4.4 Hz, 1H), 7.74 (dd, J=2.0, 0.9 Hz, 1H), 7.34 (s, 1H), 7.15 (s, 2H), 6.95 (dd, J=7.2, 2.0 Hz, 1H), 6.10 (d, J=0.8 Hz, 1H), 4.62 (s, 1H), 3.86 (s, 2H), 2.86-2.74 (m, 1H), 2.46 (s, 3H), 2.17 (s, 6H), 1.15 (s, 6H), 0.70-0.63 (m, 2H), 0.51-0.45 (m, 2H).

### Example 327: 1-[[4-[2-[(2,6-dimethylpyrimidin-4-yl)amino]pyrazolo[1,5-a]pyridin-5-yl]-6-(fluoromethyl)-3-pyridyl]oxy]-2-methyl-propan-2-ol

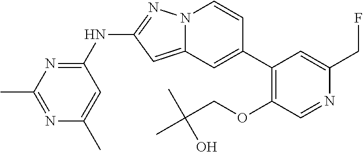 [SMILES: Cc1cc(Nc2cc3cc(-c4cc(CF)ncc4OCC(C)(C)O)ccn3n2)nc(C)n1]

The title compound was prepared by the similar method of Example 1 using 2-methylpropane-1,2-diol instead of (R)-(1-methylpyrrolidin-3-yl)methanol and N-(2,6-dimethylpyrimidin-4-yl)-5-(5-fluoro-2-(fluoromethyl)pyridin-4-yl)pyrazolo[1,5-a]pyridin-2-amine (made in similar method as Intermediate 15) instead of N-(5-(5-chloro-2-cyanopyridin-4-yl)pyrazolo[1,5-a]pyridin-2-yl)cyclopropanecarboxamide (8 mg, 13.4%). MS (ESI): mass calcd. for C₂₃H₂₅FN₆O₂, 436.2. m/z found, 437.2 [M+H]⁺. ¹H NMR (400 MHz, DMSO-d6) δ 10.21 (s, 1H), 8.61 (dt, J=7.2, 0.9 Hz, 1H), 8.50 (s, 1H), 7.92 (dd, J=2.1, 0.9 Hz, 1H), 7.65 (s, 1H), 7.10 (dd, J=7.2, 2.0 Hz, 1H), 7.03 (s, 1H), 6.85 (s, 1H), 5.45 (d, J=47.6 Hz, 2H), 4.67 (s, 1H), 3.97 (s, 2H), 2.46 (s, 3H), 2.30 (s, 3H), 1.17 (s, 6H).

### Example 328: 3-[[4-[2-[(2,6-dimethylpyrimidin-4-yl)amino]pyrazolo[1,5-a]pyridin-5-yl]-6-ethyl-3-pyridyl]oxy]-2,2-dimethyl-propanenitrile

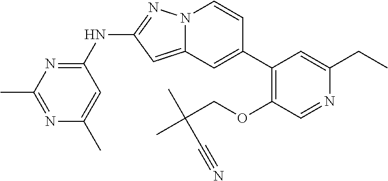 [SMILES: CCc1cc(-c2ccn3nc(Nc4cc(C)nc(C)n4)cc3c2)c(OCC(C)(C)C#N)cn1]

The title compound was prepared by the similar method of Example 1 using 3-hydroxy-2,2-dimethylpropanenitrile instead of (R)-(1-methylpyrrolidin-3-yl)methanol and N-(2,6-dimethylpyrimidin-4-yl)-5-(2-ethyl-5-fluoropyridin-4-yl)pyrazolo[1,5-a]pyridin-2-amine (made in similar method as Intermediate 15) instead of N-(5-(5-chloro-2-cyanopyridin-4-yl)pyrazolo[1,5-a]pyridin-2-yl)cyclopropanecarboxamide. (17 mg, 9.3%). MS (ESI): mass calcd. for C₂₅H₂₇N₇O, 441.2. m/z found, 442.2 [M+H]⁺. ¹H NMR (500 MHz, DMSO-d6) δ 10.20 (s, 1H), 8.59 (dd, J=7.2, 0.9 Hz, 1H), 8.39 (s, 1H), 7.88 (dd, J=2.0, 0.9 Hz, 1H), 7.40 (s, 1H), 7.08-7.01 (m, 2H), 6.85 (s, 1H), 4.20 (s, 2H), 2.77 (q, J=7.6 Hz, 2H), 2.45 (s, 3H), 2.30 (s, 3H), 1.36 (s, 6H), 1.25 (t, J=7.6 Hz, 3H).

### Example 329: 3-[[6-(difluoromethoxy)-4-[2-[(2,6-dimethylpyrimidin-4-yl)amino]pyrazolo[1,5-a]pyridin-5-yl]-3-pyridyl]oxy]-2,2-dimethyl-propanenitrile

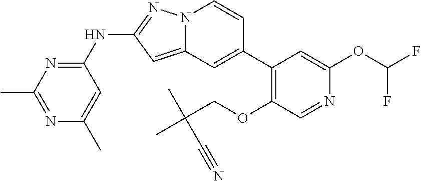 [SMILES: Cc1cc(Nc2cc3cc(-c4cc(OC(F)F)ncc4OCC(C)(C)C#N)ccn3n2)nc(C)n1]

Step A: 3-((4-iodo-6-methoxypyridin-3-yl)oxy)-2,2-dimethylpropanenitrile. To a mixture of 4-iodo-6-methoxypyridin-3-ol (20 g, 79.7 mmol, 1.00 equiv), 2-cyano-2-methylpropyl-4-methylbenzenesulfonate (24.2 g, 95.6 mmol, 1.20 equiv), Cs₂CO₃ (62.3 g, 191 mmol, 2.40 equiv) in DMA (640 mL, 0.125M) was heated at 90° C. for 14 hours. The reaction mixture was cooled to 23° C. and water (400 mL) was added followed by EtOAc (400 mL). The layers were separated and the aqueous was extracted with EtOAc (2×400 mL). The combined organics were washed with brine (2×300 mL) and then dried with MgSO₄, filtered and concentrated in vacuo. The resulting oil was then purified by silica gel chromatography (0-50% EtOAc in hexanes) to give 3-((4-iodo-6-methoxypyridin-3-yl)oxy)-2,2-dimethylpropanenitrile (18 g, 54.2 mmol, 68% yield) as a pale yellow oil.

Step B: 3-((6-hydroxy-4-iodopyridin-3-yl)oxy)-2,2-dimethylpropanenitrile. To a suspension of 3-((4-iodo-6-methoxypyridin-3-yl)oxy)-2,2-dimethylpropanenitrile (18 g, 54.2 mmol, 1.00 equiv) and sodium iodide (32.5 g, 216 mmol, 4.00 equiv) in MeCN (540 mL, 0.10M) was added TMSCl (34.4 mL, 271 mmol, 5.00 equiv). The resulting mixture was heated at reflux for 3 hours and then the mixture cooled to 23° C. To the mixture was added EtOAc (500 mL) and then washed with saturated aqueous NH₄Cl (2×250 mL) followed by water (250 mL). The organics were concentrated in vacuo and then purified by silica gel chromatography (10% 2N NH₄OH/MeOH in DCM) to give 3-((6-hydroxy-4-iodopyridin-3-yl)oxy)-2,2-dimethylpropanenitrile (14.4 g, 45.3 mmol, 83% yield) as a beige solid.

Step C: 3-((6-(difluoromethoxy)-4-iodopyridin-3-yl)oxy)-2,2-dimethylpropanenitrile. To solution of 3-((6-hydroxy-4-iodopyridin-3-yl)oxy)-2,2-dimethylpropanenitrile (14.4 g, 45.3 mmol, 1.00 equiv), diethyl-(bromodifluoromethyl)phosphonate (8.9 mL, 50.4 mmol, 1.1 equiv) in MeCN (250 mL, 0.18 M) was added potassium fluoride (5.20 g, 90 mmol, 2.00 equiv) and the resulting mixture was heated at 60° C. for 16 hours. The mixture was then cooled to 23° C. and diluted with EtOAc (500 mL) and then washed with brine (3×300 mL). The organics were then concentrated in vacuo and purified by silica gel chromatography (10-40% EtOAc in hexanes) to give 3-((6-(difluoromethoxy)-4-iodopyridin-3-yl)oxy)-2,2-dimethylpropanenitrile (15.5 g, 42 mmol, 93% yield) as a clear colorless oil.

Step D: 3-[[6-(difluoromethoxy)-4-[2-[(2,6-dimethylpyrimidin-4-yl)amino]pyrazolo[1,5-a]pyridin-5-yl]-3-pyridyl]oxy]-2,2-dimethyl-propanenitrile. To a solution of 3-((6-(difluoromethoxy)-4-iodopyridin-3-yl)oxy)-2,2-dimethylpropanenitrile (15.5 g, 42 mmol, 1.00 equiv), Intermediate 22 (16.9 g, 46.3 mmol, 1.10 equiv), Pd(dppf)Cl₂ (3.48 g, 4.20 mmol, 0.10 equiv), aq. K₃PO₄ (0.5 M, 168 mL, 1.00 equiv) and 1,4-dioxane (168 mL, 0.25 M) was purged with N₂ (evacuated and back-filled with N₂×3). The resulting mixture was heated at 60° C. for 3 hours. The mixture was then cooled to 23° C. and diluted with EtOAc:water (1:1, 1000 mL) which resulting a black mass which was then dissolved in hot DCM and then was added silica gel. The organics were removed in vacuo and the crude material was purified by silica gel chromatography (5% MeOH in DCM) to give the title compound (15.5 g, 32.3 mmol, 77% yield) as an off-white solid. MS (ESI): mass calcd. for C₂₄H₂₃F₂N₇O₂, 479.2. m/z found, 480.1 [M+H]⁺. ¹H NMR (400 MHz, DMSO-d6) δ 10.23 (s, 1H), 8.62 (dt, J=7.3, 0.9 Hz, 1H), 8.15 (s, 1H), 7.93 (dd, J=2.0, 0.9 Hz, 1H), 7.68 (t, J=73.1 Hz, 1H), 7.28 (s, 1H), 7.10-6.98 (m, 2H), 6.88 (s, 1H), 4.22 (s, 2H), 2.46 (s, 3H), 2.31 (s, 3H), 1.36 (s, 6H). ¹⁹F NMR (400 MHz, DMSO-d6), −86.44 ppm.

### Example 330: 4-[[5-[5-(4-hydroxycyclohexoxy)-2-methyl-4-pyridyl]pyrazolo[1,5-a]pyridin-2-yl]amino]-N-isopropyl-2,6-dimethoxy-benzamide

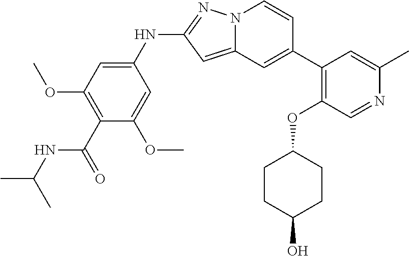 [SMILES: COc1cc(Nc2cc3cc(-c4cc(C)ncc4O[C@H]4CC[C@H](O)CC4)ccn3n2)cc(OC)c1C(=O)NC(C)C]

A solution of iPrMgCl (0.225 mL, 0.451 mmol) was added dropwise to a stirred mixture of Intermediate 23 (80 mg, 0.15 mmol), isopropylamine (44 mg, 0.75 mmol) in THF (6.9 mL), The mixture was stirred at RT for 1 h. The reaction was quenched with sat. aq. NH₄Cl (2 mL) and extracted with EtOAc (3×10 mL). The combined organics were dried with Na₂SO₄, filtered and concentrated to a residue. The residue was then purified by PTLC with 0-5% MeOH in DCM to afford the title compound (28 mg, 33%). MS (ESI): mass calcd. for C₃₁H₃₇N₅O₅, 559.3. m/z found, [M+H]⁺. ¹H NMR (400 MHz, Methanol-d4) δ 8.29 (d, J=7.2 Hz, 1H), 8.13 (s, 1H), 7.53 (d, J=2.0 Hz, 1H), 7.25 (s, 1H), 6.83 (dd, J=7.3, 1.9 Hz, 1H), 6.74 (s, 2H), 6.07 (s, 1H), 4.36-4.17 (m, 1H), 4.10-3.97 (m, 1H), 3.73 (s, 6H), 3.57-3.42 (m, 1H), 2.42 (s, 3H), 2.04-1.90 (m, 2H), 1.77 (t, J=8.7 Hz, 2H), 1.47-1.35 (m, 2H), 1.35-1.21 (m, 2H), 1.11 (d, J=6.6 Hz, 6H).

### Example 331: 4-[[5-[5-(4-hydroxycyclohexoxy)-2-methyl-4-pyridyl]pyrazolo[1,5-a]pyridin-2-yl]amino]-2,6-dimethoxy-N-methyl-benzamide

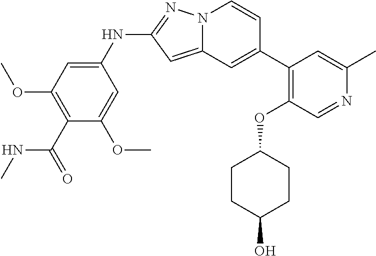

The title compound was prepared by the similar method of Example 330 using methylamine instead of isopropylamine (40 mg, 36%). MS (ESI): mass calcd. for C₂₉H₃₃N₅O₅, 531.2. m/z found, 532.1 [M+H]⁺. ¹H NMR (500 MHz, Methanol-d4) δ 8.47-8.35 (m, 1H), 8.25 (s, 1H), 7.66 (dd, J=2.1, 0.9 Hz, 1H), 7.38 (s, 1H), 6.96 (dd, J=7.2, 1.9 Hz, 1H), 6.87 (s, 2H), 6.20 (s, 1H), 4.45-4.36 (m, 1H), 3.85 (s, 6H), 3.71-3.62 (m, 1H), 2.88 (s, 3H), 2.54 (s, 3H), 2.14-2.05 (m, 2H), 1.93-1.86 (m, 2H), 1.59-1.50 (m, 2H), 1.46-1.35 (m, 2H).

### Example 332: 4-[[5-[5-(4-hydroxycyclohexoxy)-2-methyl-4-pyridyl]pyrazolo[1,5-a]pyridin-2-yl]amino]-2,6-dimethoxy-N,N-dimethyl-benzamide

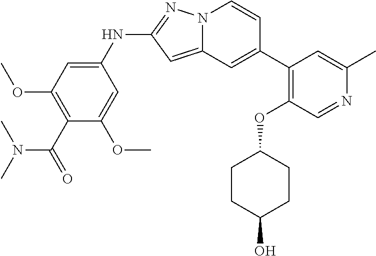

The title compound was prepared by the similar method of Example 330 using dimethylamine instead of isopropylamine (31 mg, 46.5%). MS (ESI): mass calcd. for C₃₀H₃₅N₅O₅, 545.3. m/z found, 546.2 [M+H]⁺. ¹H NMR (500 MHz, Methanol-d4) δ 8.27 (d, J=7.2 Hz, 1H), 8.12 (s, 1H), 7.54-7.49 (m, 1H), 7.23 (s, 1H), 6.81 (dd, J=7.2, 1.9 Hz, 1H), 6.74 (s, 2H), 6.09-5.98 (m, 1H), 4.32-4.19 (m, 1H), 3.72 (s, 6H), 3.56-3.49 (m, 1H), 3.48-3.42 (m, 2H), 2.41 (s, 3H), 2.01-1.93 (m, 2H), 1.81-1.73 (m, 6H), 1.45-1.37 (m, 2H), 1.32-1.24 (m, 2H).

### Example 333: N-cyclopropyl-2-(difluoromethoxy)-4-[[5-[5-(4-hydroxycyclohexoxy)-2-methyl-4-pyridyl]pyrazolo[1,5-a]pyridin-2-yl]amino]-6-methoxy-benzamide

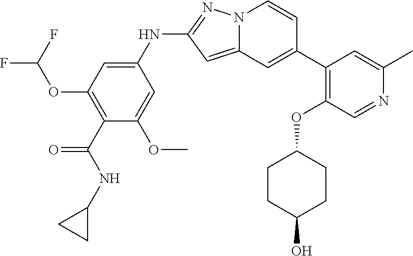 [SMILES: COc1cc(Nc2cc3cc(-c4cc(C)ncc4O[C@H]4CC[C@H](O)CC4)ccn3n2)cc(OC(F)F)c1C(=O)NC1CC1]

The title compound was prepared as described in Example 239 where 4-bromo-N-cyclopropyl-2-(difluoromethoxy)-6-methoxy-benzamide was used instead of 6-bromo-1H-pyrrolo[2,3-b]pyridine (125 mg, 57%). MS (ESI): mass calcd. for C₃₁H₃₃F₂N₅O₅, 593.2. m/z found, [M+H]⁺. ¹H NMR (600 MHz, Methanol-d4) δ 8.44-8.32 (m, 1H), 8.23 (s, 1H), 7.69-7.58 (m, 1H), 7.34 (s, 1H), 7.14 (d, J=1.8 Hz, 1H), 7.04-6.99 (m, 1H), 6.99-6.92 (m, 1H), 6.77 (t, J=74.4 Hz, 1H), 6.16 (s, 1H), 4.45-4.33 (m, 1H), 3.88 (s, 3H), 3.70-3.60 (m, 1H), 2.91-2.72 (m, 1H), 2.53 (s, 3H), 2.12-2.04 (m, 2H), 1.96-1.79 (m, 3H), 1.57-1.47 (m, 2H), 1.45-1.37 (m, 2H), 0.83-0.74 (m, 2H), 0.67-0.60 (m, 2H).

### Example 334: N-cyclopropyl-6-[[5-[5-(4-hydroxycyclohexoxy)-2-methyl-4-pyridyl]pyrazolo[1,5-a]pyridin-2-yl]amino]-2,4-dimethyl-pyridine-3-carboxamide

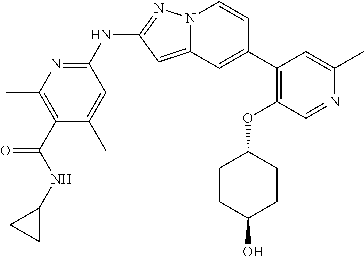

The title compound was prepared as described in Example 239 where 6-chloro-N-cyclopropyl-2,4-dimethylnicotinamide was used instead of 6-bromo-1H-pyrrolo[2,3-b]pyridine (91 mg, 53%). MS (ESI): mass calcd. for C₃₀H₃₄N₆O₃, 526.3. m/z found, 527.3 [M+H]⁺. ¹H NMR (500 MHz, Methanol-d4) δ 8.50-8.35 (m, 1H), 8.25 (s, 1H), 7.71 (dd, J=1.9, 0.9 Hz, 1H), 7.38 (s, 1H), 7.08 (s, 1H), 6.99 (dd, J=7.2, 1.9 Hz, 1H), 6.81 (s, 1H), 4.45-4.35 (m, 1H), 3.71-3.60 (m, 1H), 2.97-2.79 (m, 1H), 2.54 (s, 3H), 2.44 (s, 3H), 2.29 (s, 3H), 2.14-1.98 (m, 2H), 1.94-1.83 (m, 2H), 1.61-1.33 (m, 4H), 0.90-0.80 (m, 2H), 0.69-0.53 (m, 2H).

### Example 335: N-cyclopropyl-6-[[5-[5-(2-hydroxy-2-methyl-propoxy)-2-methyl-4-pyridyl]pyrazolo[1,5-a]pyridin-2-yl]amino]-2,4-dimethyl-pyridine-3-carboxamide

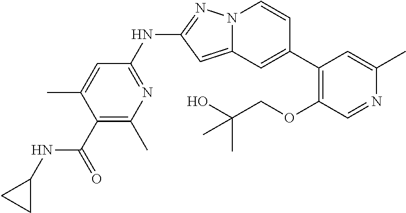 [SMILES: Cc1cc(-c2ccn3nc(Nc4cc(C)c(C(=O)NC5CC5)c(C)n4)cc3c2)c(OCC(C)(C)O)cn1]

The title compound was prepared as described in Example 239 where 6-chloro-N-cyclopropyl-2,4-dimethylnicotinamide was used instead of 6-bromo-1H-pyrrolo[2,3-b]pyridine (75 mg, 39%). MS (ESI): mass calcd. for C₂₈H₃₂N₆O₃, 500.3. m/z found, 501.2 [M+H]⁺. ¹H NMR (500 MHz, Chloroform-d) δ 8.21 (d, J=7.1 Hz, 1H), 8.00 (s, 1H), 7.85 (s, 1H), 7.37 (s, 1H), 6.98 (s, 1H), 6.78-6.62 (m, 3H), 6.30 (s, 1H), 3.81 (s, 2H), 3.44 (s, 1H), 2.87-2.78 (m, 1H), 2.41 (s, 3H), 2.30 (s, 3H), 2.09 (s, 3H), 1.22 (s, 6H), 0.83-0.72 (m, 2H), 0.56 (s, 6H), 0.62-0.51 (m, 2H).

### Example 336: (1S,5R)-7-[[4-[2-[(2,6-dimethylpyrimidin-4-yl)amino]pyrazolo[1,5-a]pyridin-5-yl]-6-methyl-3-pyridyl]oxy]-3-oxa-9-azabicyclo[3.3.1]nonane-9-carbonitrile

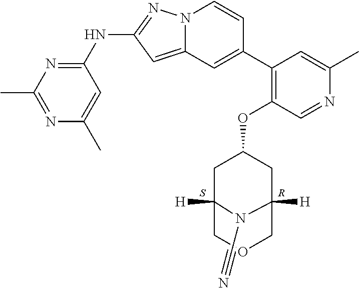

To a mixture of N-(2,6-dimethylpyrimidin-4-yl)-5-[2-methyl-5-[[(1S,5R)-3-oxa-9-azabicyclo[3.3.1]nonan-7-yl]oxy]-4-pyridyl]pyrazolo[1,5-a]pyridin-2-amine (Intermediate 24) (50 mg, 0.10 mmol), NaHCO₃ (26 mg, 0.31 mmol), and MeOH (5 mL) was added cyanogen bromide (13.1 mg, 0.12 mmol). The solution was stirred at room temperature for 16 h. To the mixture was added 1 mL sat. aq. NaHCO₃ solution. The reaction mixture was extracted with DCM (5×2 mL). The organic layer was concentrated down under vacuo. The crude material was purified with PTLC using 5-10% MeOH in DCM as eluent to afford the title compound (31 mg, 60%). MS (ESI): mass calcd. for C₂₇H₂₈N₈O₂, 496.2. m/z found, 497.3 [M+H]⁺. ¹H NMR (500 MHz, Methanol-d4) δ 8.27 (d, J=7.3 Hz, 1H), 8.05 (s, 1H), 7.87 (d, J=1.8 Hz, 1H), 7.25 (s, 1H), 7.01 (dd, J=7.3, 2.1 Hz, 1H), 6.90 (s, 1H), 6.69 (s, 1H), 4.59 (dd, J=6.1, 3.0 Hz, 1H), 3.83-3.59 (m, 4H), 3.39 (d, J=6.8 Hz, 2H), 2.49-2.37 (m, 8H), 2.26 (s, 3H), 1.97-1.88 (m, 2H).

### Example 337: 2,4-dimethyl-6-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]pyridazin-3-one

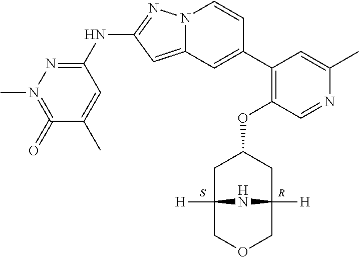

The title compound was prepared by the similar method of Example 1 using (1R,5S,7s)-3-oxa-9-azabicyclo[3.3.1]nonan-7-ol instead of (R)-(1-methylpyrrolidin-3-yl)methanol and 6-((5-(5-fluoro-2-methylpyridin-4-yl)pyrazolo[1,5-a]pyridin-2-yl)amino)-2,4-dimethylpyridazin-3(2H)-one (made in similar method as Intermediate 15) instead of N-(5-(5-chloro-2-cyanopyridin-4-yl)pyrazolo[1,5-a]pyridin-2-yl)cyclopropanecarboxamide (37 mg, 13%). MS (ESI): mass calcd. for C₂₆H₂₉N₇O₃, 487.2. m/z found, [M+H]⁺. ¹H NMR (500 MHz, Methanol-d4) δ 8.25 (d, J=7.2 Hz, 1H), 8.06 (s, 1H), 7.99-7.90 (m, 1H), 7.29 (s, 1H), 7.20-7.14 (m, 1H), 7.06 (dd, J=7.2, 2.0 Hz, 1H), 6.73 (d, J=0.8 Hz, 1H), 4.61-4.56 (m, 1H), 3.70-3.59 (m, 8H), 2.92-2.78 (m, 3H), 2.42 (s, 3H), 2.32-2.23 (m, 2H), 2.10 (d, J=1.1 Hz, 3H), 1.90-1.83 (m, 2H).

### Example 338: N-(3-fluoro-4-methyl-2-pyridyl)-5-[2-methyl-5-[[(1S,5R)-3-oxa-9-azabicyclo[3.3.1]nonan-7-yl]oxy]-4-pyridyl]pyrazolo[1,5-a]pyridin-2-amine

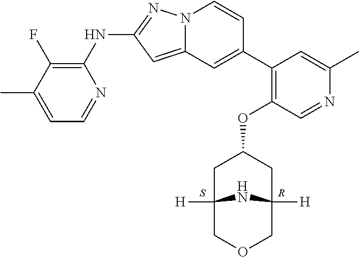

The title compound was prepared as described in Example 239 where 2-bromo-3-fluoro-4-picoline was used instead of 6-bromo-1H-pyrrolo[2,3-b]pyridine and 5-(5-(((1R,5S,7s)-3-oxa-9-azabicyclo[3.3.1]nonan-7-yl)oxy)-2-methylpyridin-4-yl)pyrazolo[1,5-a]pyridin-2-amine was used instead of (1r,4r)-4-((4-(2-aminopyrazolo[1,5-a]pyridin-5-yl)-6-methylpyridin-3-yl)oxy)cyclohexan-1-ol (Intermediate 12). The preparation of 5-(5-(((1R,5S,7s)-3-oxa-9-azabicyclo[3.3.1]nonan-7-yl)oxy)-2-methylpyridin-4-yl)pyrazolo[1,5-a]pyridin-2-amine is similar to Intermediate 12 (12 mg, 10.9%). MS (ESI): mass calcd. for C₂₆H₂₇FN₆O₂, 474.2. m/z found, 475.1 [M+H]⁺. ¹H NMR (500 MHz, Methanol-d4) δ 8.40-8.30 (m, 1H), 8.15 (s, 1H), 8.03 (dd, J=2.0, 0.9 Hz, 1H), 7.89 (d, J=5.1 Hz, 1H), 7.39 (s, 1H), 7.14 (dd, J=7.2, 2.0 Hz, 1H), 7.00 (d, J=0.8 Hz, 1H), 6.72 (t, J=5.1 Hz, 1H), 4.70-4.55 (m, 1H), 3.81-3.66 (m, 4H), 2.96 (d, J=7.4 Hz, 2H), 2.52 (s, 3H), 2.41-2.26 (m, 5H), 1.99-1.91 (m, 2H).

### Example 339: N-[5-[5-[[(1S,5R)-9-acetyl-3-oxa-9-azabicyclo[3.3.1]nonan-7-yl]oxy]-2-methyl-4-pyridyl]pyrazolo[1,5-a]pyridin-2-yl]cyclopropanecarboxamide

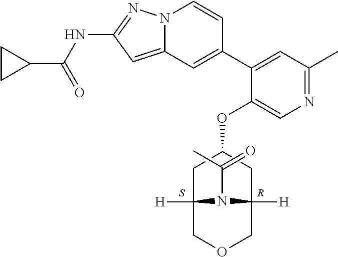

Step A. N-(5-(5-(((1R,5S,7s)-3-oxa-9-azabicyclo[3.3.1]nonan-7-yl)oxy)-2-methylpyridin-4-yl)pyrazolo[1,5-a]pyridin-2-yl)cyclopropanecarboxamide. To a 0° C. solution of N-(5-(5-fluoro-2-methylpyridin-4-yl)pyrazolo[1,5-a]pyridin-2-yl)cyclopropanecarboxamide (Intermediate 6) (300 mg, 0.967 mmol) and DMA (2 mL) was added NaH (60% dispersion in mineral oil) (116 mg, 2.9 mmol). The reaction mixture was stirred for 1.5 h at room temperature and 90° C. for 1 h. The reaction was cooled to room temperature and then quenched with aqueous saturated NH₄OH solution. Then the reaction mixture was extracted with DCM and the organics were concentrated down under vacuo. The crude was purified by FCC using MeOH/DCM as solvent. The pure fraction was collected and concentrated down to afford the title compound (200 mg, 47.7%). MS (ESI): mass calcd. for C₂₄H₂₇N₅O₃, 433.5. m/z found, 434.2 [M+H]⁺. 1H NMR (500 MHz, Methanol-d4) δ 8.33 (d, J=7.3 Hz, 1H), 8.12 (s, 1H), 8.05 (d, J=1.9 Hz, 1H), 7.31 (s, 1H), 7.14 (dd, J=7.3, 2.0 Hz, 1H), 6.84 (s, 1H), 4.70-4.63 (m, 2H), 3.80-3.61 (m, 4H), 2.96 (d, J=7.1 Hz, 2H), 2.49 (s, 3H), 2.41-2.31 (m, 2H), 1.96-1.81 (m, 3H), 1.04-0.96 (m, 2H), 0.95-0.79 (m, 2H).

Step B. N-(5-(5-(((1R,5S,7s)-9-acetyl-3-oxa-9-azabicyclo[3.3.1]nonan-7-yl)oxy)-2-methylpyridin-4-yl)pyrazolo[1,5-a]pyridin-2-yl)cyclopropanecarboxamide. To a 0° C. solution of N-(5-(5-(((1R,5S,7s)-3-oxa-9-azabicyclo[3.3.1]nonan-7-yl)oxy)-2-methylpyridin-4-yl)pyrazolo[1,5-a]pyridin-2-yl)cyclopropanecarboxamide (100 mg, 0.23 mmol) in DCM/DMF (10 mL/1 mL) and TEA (0.962 mL, 0.692 mmol) was added, acetic acid (16.6 mg, 0.28 mmol) and HATU (114 mg, 0.3 mmol). The solution was stirred for 2 h. LCMS showed desired mass peak. The mixture was added 1 mL saturated aqueous NaHCO₃ solution. The mixture was extracted with DCM (2×2 mL). The organic layers were combined and concentrated under vacuo. The crude was purified via PTLC with 5-10% MeOH in DCM to afford the title compound (98 mg, 89.3%). MS (ESI): mass calcd. for C₂₆H₂₉N₅O₄, 475.5. m/z found, 476.1 [M+H]⁺. ¹H NMR (500 MHz, Methanol-d4) δ 8.39-8.31 (m, 1H), 8.12 (s, 1H), 8.06 (dd, J=2.0, 0.9 Hz, 1H), 7.34 (s, 1H), 7.15 (dd, J=7.3, 2.0 Hz, 1H), 6.85 (s, 1H), 4.57-4.48 (m, 2H), 4.04-3.94 (m, 1H), 3.81-3.71 (m, 2H), 3.69-3.55 (m, 2H), 2.50 (s, 3H), 2.42-2.33 (m, 1H), 2.29-2.22 (m, 1H), 2.12-1.93 (m, 5H), 1.89-1.82 (m, 1H), 1.02-0.96 (m, 2H), 0.93-0.85 (m, 2H).

### Example 340: N-isopropyl-2,6-dimethoxy-4-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]benzamide

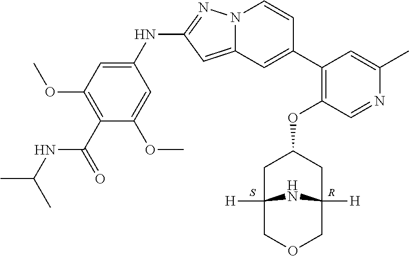

The title compound (25 mg, 28%) was prepared as described in Example 239 where 4-bromo-N-isopropyl-2,6-dimethoxybenzamide was used instead of 6-bromo-1H-pyrrolo[2,3-b]pyridine and 5-(5-(((1R,5S,7s)-3-oxa-9-azabicyclo[3.3.1]nonan-7-yl)oxy)-2-methylpyridin-4-yl)pyrazolo[1,5-a]pyridin-2-amine was used instead of (1r,4r)-4-((4-(2-aminopyrazolo[1,5-a]pyridin-5-yl)-6-methylpyridin-3-yl)oxy)cyclohexan-1-ol (Intermediate 12). The preparation of 5-(5-(((1R,5S,7s)-3-oxa-9-azabicyclo[3.3.1]nonan-7-yl)oxy)-2-methylpyridin-4-yl)pyrazolo[1,5-a]pyridin-2-amine is similar to Intermediate 12. MS (ESI): mass calcd. for C₃₂H₃₈N₆O₅, 586.3. m/z found, 587.3 [M+H]⁺. ¹H NMR (500 MHz, Methanol-d4) δ 8.41-8.22 (m, 1H), 8.14 (s, 1H), 7.96 (dd, J=2.0, 0.9 Hz, 1H), 7.36 (s, 1H), 7.10-7.00 (m, 1H), 6.83 (s, 2H), 6.13 (d, J=0.8 Hz, 1H), 4.70-4.61 (m, 1H), 4.19-4.05 (m, 1H), 3.87-3.61 (m, 10H), 2.95 (d, J=7.2 Hz, 2H), 2.51 (s, 3H), 2.40-2.27 (m, 2H), 1.99-1.88 (m, 2H), 1.21 (d, J=6.6 Hz, 6H).

### Example 341: N-isopropyl-2-methyl-5-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]pyrazole-3-carboxamide

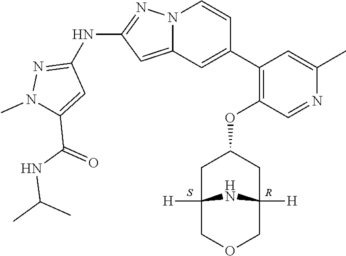

The title compound was prepared as described in Example 239 where 3-bromo-N-isopropyl-1-methyl-1H-pyrazole-5-carboxamide was used instead of 6-bromo-1H-pyrrolo[2,3-b]pyridine and 5-(5-(((1R,5S,7s)-3-oxa-9-azabicyclo[3.3.1]nonan-7-yl)oxy)-2-methylpyridin-4-yl)pyrazolo[1,5-a]pyridin-2-amine was used instead of (1r,4r)-4-((4-(2-aminopyrazolo[1,5-a]pyridin-5-yl)-6-methylpyridin-3-yl)oxy)cyclohexan-1-ol (Intermediate 12). The preparation of 5-(5-(((1R,5S,7s)-3-oxa-9-azabicyclo[3.3.1]nonan-7-yl)oxy)-2-methylpyridin-4-yl)pyrazolo[1,5-a]pyridin-2-amine is similar to Intermediate 12 (16 mg, 20%). MS (ESI): mass calcd. for C₂₈H₃₄N₈O₃, 530.3. m/z found, 531.2 [M+H]⁺. ¹H NMR (400 MHz, Methanol-d4) δ 8.40-8.29 (m, 1H), 8.16 (s, 1H), 7.93 (dd, J=2.0, 0.9 Hz, 1H), 7.44-7.35 (m, 1H), 7.06 (dd, J=7.2, 2.0 Hz, 1H), 6.73 (s, 1H), 6.31 (d, J=0.8 Hz, 1H), 4.73-4.62 (m, 1H), 4.24-4.12 (m, 1H), 4.00 (s, 3H), 3.82-3.62 (m, 4H), 3.01-2.94 (m, 2H), 2.51 (s, 3H), 2.41-2.29 (m, 2H), 1.99-1.86 (m, 2H), 1.25 (d, J=6.6 Hz, 6H).

### Example 342: (4R)-4-[[4-[2-[(2,6-dimethylpyrimidin-4-yl)amino]pyrazolo[1,5-a]pyridin-5-yl]-6-methyl-3-pyridyl]oxymethyl]oxazolidin-2-one

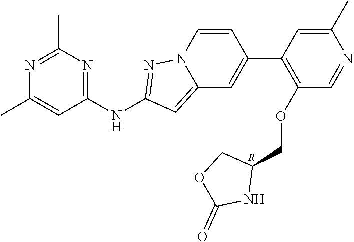

The title compound was prepared as described in Example 203 where (S)-(2-oxooxazolidin-4-yl)methyl 4-methylbenzenesulfonate was used instead of (3-hydroxytetrahydrofuran-3-yl)methyl 4-methylbenzenesulfonate (88.5 mg, 67.6%). MS (ESI): mass calcd. for C₂₃H₂₃N₇O₃, 445.2. m/z found, 446.2 [M+H]⁺. ¹H NMR (400 MHz, DMSO-d6) δ 10.21 (s, 1H), 8.50 (d, J=7.2 Hz, 1H), 8.36 (s, 1H), 7.91 (s, 1H), 7.85 (d, J=1.6 Hz, 1H), 7.40 (s, 1H), 7.06-7.03 (m, 2H), 6.88 (s, 1H), 4.44-4.36 (m, 1H), 4.19-4.12 (m, 4H), 2.47 (s, 3H), 2.46 (s, 3H), 2.30 (s, 3H).

### Example 343: 4-[[4-[2-[(2,6-dimethylpyrimidin-4-yl)amino]pyrazolo[1,5-a]pyridin-5-yl]-6-methyl-3-pyridyl]oxy]cyclohexanecarboxamide

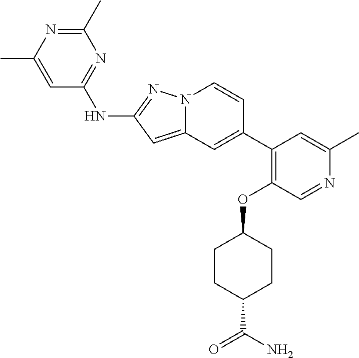

Step A. methyl (1r,4r)-4-((4-(2-((2,6-dimethylpyrimidin-4-yl)amino)pyrazolo[1,5-a]pyridin-5-yl)-6-methylpyridin-3-yl)oxy)cyclohexane-1-carboxylate. The title compound was prepared as described in Example 203 where (cis)-methyl 4-(tosyloxy)cyclohexanecarboxylate was used instead of (3-hydroxytetrahydrofuran-3-yl)methyl 4-methylbenzenesulfonate (88 mg, 20.9%). MS (ESI): mass calcd. for C₂₇H₃₀N₆O₃, 486.6. m/z found, 487.3 [M+H]+. ¹H NMR (400 MHz, DMSO-d6) δ 10.22 (s, 1H), 8.58 (d, J=7.6 Hz, 1H), 8.40-8.34 (m, 1H), 7.84-7.77 (m, 1H), 7.38-7.33 (m, 1H), 7.04-6.98 (m, 2H), 6.87 (s, 1H), 4.46-4.31 (m, 1H), 3.58 (s, 3H), 2.48-2.44 (m, 6H), 2.31 (s, 3H), 2.10-1.99 (m, 2H), 1.93-1.82 (m, 2H), 1.66-1.57 (m, 2H), 1.52-1.37 (m, 3H)

Step B. (1r,4r)-4-((4-(2-((2,6-dimethylpyrimidin-4-yl)amino)pyrazolo[1,5-a]pyridin-5-yl)-6-methylpyridin-3-yl)oxy)cyclohexane-1-carboxylic acid. A solution consisting of (trans)-methyl 4-((4-(2-((2,6-dimethylpyrimidin-4-yl)amino)pyrazolo[1,5-a]pyridin-5-yl)-6-methylpyridin-3-yl)oxy)cyclohexanecarboxylate (78.0 mg, 0.160 mmol), LiOH·H₂O (10.1 mg, 0.240 mmol), THF (0.5 mL), H₂O (0.5 mL) and MeOH (0.5 mL) was stirred at room-temperature for 1 h. The reaction mixture concentrated to dryness under reduced pressure to give the crude product. Then added hydrated citric acid solution, during this period, yellow solid was formed. It was collected by filtration to give the crude product (100 mg, 132%) as a yellow oil. ¹H NMR (400 MHz, DMSO-d6) δ 11.56 (br. s., 1H), 10.22 (s, 1H), 8.58 (d, J=7.2 Hz, 1H), 8.38 (s, 1H), 7.79 (s, 1H), 7.35 (s, 1H), 7.05-6.98 (m, 2H), 6.90-6.85 (m, 1H), 4.43-4.33 (m, 1H), 2.47-2.46 (m, 6H), 2.31 (s, 3H), 2.08-2.00 (m, 2H), 1.92-1.84 (m, 2H), 1.66-1.59 (m, 1H), 1.52-1.37 (m, 4H).

Step C. (1r,4r)-4-((4-(2-((2,6-dimethylpyrimidin-4-yl)amino)pyrazolo[1,5-a]pyridin-5-yl)-6-methylpyridin-3-yl)oxy)cyclohexane-1-carboxamide. A mixture consisting of (trans)-4-((4-(2-((2,6-dimethylpyrimidin-4-yl)amino)pyrazolo[1,5-a]pyridin-5-yl)-6-methylpyridin-3-yl)oxy)cyclohexanecarboxylic acid (80.0 mg, 0.169 mmol), NH₄Cl (18.1 mg, 0.339 mmol), DIEA (43.8 mg, 0.339 mmol), HATU (77.2 mg, 0.203 mmol) and DMF (3 mL) was stirred at room-temperature for 4 hours. The crude product was purified by preparative HPLC using a Phenomenex Gemini-NX 80×40 mm×3 μm column (eluent: 20% to 50% (v/v) CH₃CN and H₂O with 0.05% NH₃-H₂O+10 mM NH₄HCO₃) to afford pure product. The product was suspended in water (10 mL), the mixture frozen using dry ice/ethanol, and then lyophilized to dryness to afford the crude product as a white solid. The crude product was further purified by SFC over DAICEL CHIRALCEL OD-H 250 mm×30 mm×5 μm (eluent: 35% to 35% (v/v) EtOH with 0.1% NH₃·H₂O in supercritical CO₂). The pure fractions were collected, and the volatiles were removed under reduced pressure. The product was suspended in water (10 mL), the mixture frozen using dry ice/EtOH, and then lyophilized to dryness to afford fraction the title compound as white solid (14.8 mg, total yield: 15%). MS (ESI): mass calcd. for C₂₆H₂₉N₇O₂, 471.2. m/z found, 472.3 [M+H]⁺. ¹H NMR (400 MHz, DMSO-d6) δ 10.22 (s, 1H), 8.58 (d, J=7.2 Hz, 1H), 8.38 (s, 1H), 7.79 (d, J=0.8 Hz, 1H), 7.35 (s, 1H), 7.20 (s, 1H), 7.04-6.97 (m, 2H), 6.86 (br s, 1H), 6.69 (br s, 1H), 4.37-4.25 (m, 1H), 2.47-2.45 (m, 6H), 2.30 (s, 3H), 2.14-1.98 (m, 3H), 1.84-1.69 (m, 2H), 1.53-1.36 (m, 2H), 1.34-1.24 (m, 2H).

### Example 344: N-(2,6-dimethylpyrimidin-4-yl)-5-[5-[(3R)-1,1-dioxothiolan-3-yl]oxy-2-methyl-4-pyridyl]pyrazolo[1,5-a]pyridin-2-amine

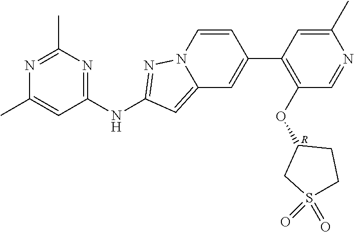

Step A. N-(2,6-dimethylpyrimidin-4-yl)-5-(2-methyl-5-((tetrahydrothiophen-3-yl)oxy)pyridin-4-yl)pyrazolo[1,5-a]pyridin-2-amine. The title compound was prepared by the similar method of Example 1 using (RS)-tetrahydrothiophen-3-ol instead of (R)-(1-methylpyrrolidin-3-yl)methanol and N-(2,6-dimethylpyrimidin-4-yl)-5-(5-fluoro-2-methylpyridin-4-yl)pyrazolo[1,5-a]pyridin-2-amine (Intermediate 15) instead of N-(5-(5-chloro-2-cyanopyridin-4-yl)pyrazolo[1,5-a]pyridin-2-yl)cyclopropanecarboxamide (500 mg, 75%, 50% pure).

Step B. R-3-((4-(2-((2,6-dimethylpyrimidin-4-yl)amino)pyrazolo[1,5-a]pyridin-5-yl)-6-methylpyridin-3-yl)oxy)tetrahydrothiophene 1,1-dioxide. Oxone (805 mg, 1.31 mmol) was added to a mixture of (RS)—N-(2,6-dimethylpyrimidin-4-yl)-5-(2-methyl-5-((tetrahydrothiophen-3-yl)oxy)pyridin-4-yl)pyrazolo[1,5-a]pyridin-2-amine (440 mg, 0.570 mmol) in THF (4 mL) and H₂O (0.8 mL) was stirred at room temperature for 1 hour. The pH was adjusted to 7 with 1M aq. NaHCO₃. The reaction mixture was poured into 50 mL of water. During this period, white precipitate was formed. It was collected by filtration and purified by preparative HPLC using a Boston Prime C18 150×30 mm×5 μm column (eluent: 30% to 60% (v/v) CH₃CN and H₂O with 0.05% NH₃-H₂O+10 mM NH₄HCO₃) to afford pure product. The product was suspended in water (10 mL), the mixture frozen using dry ice/ethanol, and then lyophilized to dryness to afford (RS)-3-((4-(2-(2,6-dimethylpyrimidin-4-yl)amino)pyrazolo[1,5-a]pyridin-5-yl)-6-methylpyridin-3-yl)oxy)tetrahydrothiophene 1,1-dioxide (70 mg, total yield: 25%) as a white solid (racemate) which was purified by SFC over Daicel Chiralcel OD-H 250 mm×30 mm×5 μm (eluent: 40% to 40% (v/v) EtOH with 0.1% NH₃·H₂O in supercritical CO₂). The pure fractions were collected, and the volatiles were removed under reduced pressure. The product was suspended in water (10 mL), the mixture frozen using dry ice/EtOH, and then lyophilized to dryness to afford the title compound (26.9 mg, 38%) as an off-white solid. MS (ESI): mass calcd. for C₂₃H₂₄N₆O₃S, 464.2. m/z found, 465.1 [M+H]⁺. ¹H NMR (400 MHz, DMSO-d6) δ 10.24 (s, 1H), 8.59 (d, J=7.2 Hz, 1H), 8.35 (s, 1H), 7.84 (d, J=1.2 Hz, 1H), 7.41 (s, 1H), 7.08-6.99 (m, 2H), 6.87 (s, 1H), 5.38-5.30 (m, 1H), 3.59-3.50 (m, 1H), 3.35-3.31 (m, 1H), 3.29-3.20 (m, 1H), 3.19-3.09 (m, 1H), 2.49 (s, 3H), 2.46 (s, 3H), 2.45-2.36 (m, 2H), 2.30 (s, 3H).

### Example 345: 4-[[5-[5-(2-hydroxy-2-methyl-propoxy)-2-methyl-4-pyridyl]pyrazolo[1,5-a]pyridin-2-yl]amino]-N-isopropyl-2-methyl-benzamide

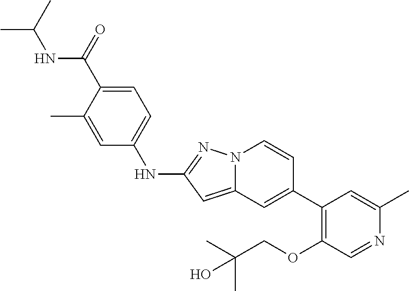 [SMILES: Cc1cc(-c2ccn3nc(Nc4ccc(C(=O)NC(C)C)c(C)c4)cc3c2)c(OCC(C)(C)O)cn1]

Step A. 4-bromo-N-isopropyl-2-methylbenzamide. A mixture consisting of 4-bromo-2-methylbenzoic acid (1 g, 5 mmol, 1 equiv.), propan-2-amine (302 mg, 5.12 mmol, 1.1 equiv.), HATU (2.1 g, 5.6 mmol, 1.2 equiv.), and DMF (10 mL) at 0° C. was stirred for 15 min. To the mixture was added DIEA (14.5 mg, 0.112 mmol, 2 equiv.) and the reaction mixture was stirred for 1 hr at room temperature. The reaction mixture was poured into water (50 mL) and a white precipitate was formed. The precipitate was collected by filtration to give the title compound (1 g, 84%) as a white solid.

Step B. 4-[[5-[5-(2-hydroxy-2-methyl-propoxy)-2-methyl-4-pyridyl]pyrazolo[1,5-a]pyridin-2-yl]amino]-N-isopropyl-2-methyl-benzamide. The title compound (99.7 mg, 42%) was prepared as described in Example 200, except 4-bromo-N-isopropyl-2-methylbenzamide (1.3 equiv.) was used instead of 4-chloro-2,6-dimethylpyrimidine in Step D. MS (ESI): mass calcd. for C₂₈H₃₃N₅O₃, 487.3. m/z found, 488.2 [M+H]⁺. ¹H NMR (400 MHz, DMSO-d6) δ 9.10 (s, 1H), 8.57 (d, J=7.2 Hz, 1H), 8.31 (s, 1H), 7.87 (d, J=8.0 Hz, 1H), 7.76 (d, J=0.8 Hz, 1H), 7.40-7.33 (m, 2H), 7.31 (d, J=1.6 Hz, 1H), 7.26 (d, J=8.4 Hz, 1H), 6.97 (dd, J=2.0, 7.2 Hz, 1H), 6.13 (s, 1H), 4.64 (s, 1H), 4.09-3.97 (m, 1H), 3.87 (s, 2H), 2.46 (s, 3H), 2.35 (s, 3H), 1.17-1.12 (m, 12H)

### Example 346: (3*R,6*S)-6-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol

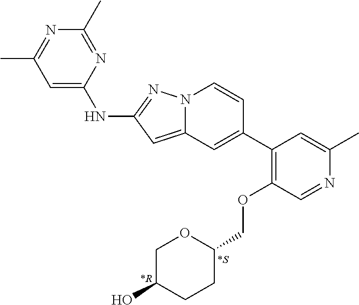

Step A. Ethyl 5-((tert-butyldimethylsilyl)oxy)tetrahydro-2H-pyran-2-carboxylate. To a solution of ethyl 5-hydroxytetrahydro-2H-pyran-2-carboxylate (4.4 g, 25 mmol, 1 equiv.) in DCM (75 mL) was added TBSCl (4.6 g, 30 mmol, 1.2 equiv.) and imidazole (4.3 g, 63 mmol, 2.5 equiv.) at 0° C. The mixture was stirred overnight then diluted with DCM (100 mL) and washed with 1N HCl (50 mL) and saturated aqueous NaHCO₃ (50 mL). The organic phase was evaporated to dryness under reduced pressure to give the crude product, ethyl 5-((tert-butyldimethylsilyl)oxy)tetrahydro-2H-pyran-2-carboxylate, as a colorless oil (6 g, 82%).

Step B. (5-((tert-butyldimethylsilyl)oxy)tetrahydro-2H-pyran-2-yl)methanol. To a solution of ethyl 5-((tert-butyldimethylsilyl)oxy)tetrahydro-2H-pyran-2-carboxylate (5.5 g, 19 mmol, 1 equiv.) and MeOH (50 mL) at 0° C. was added sodium borohydride (1.1 g, 29 mmol, 1.5 equiv.). The reaction mixture was left to stir for 2 hr at room temperature then quenched with NH₄Cl (50 mL). The reaction mixture was directly concentrated to afford the crude product, then H₂O (80 mL) was added to the mixture and the mixture was extracted with ethyl acetate (100 mL×2). The combined organic layers were washed with brine (50 mL×2), dried over sodium sulfate, then filtered and concentrated to give the title compound, (5-((tert-butyldimethylsilyl)oxy)tetrahydro-2H-pyran-2-yl)methanol, as a colorless oil (5 g, 106%).

Step C. (5-((tert-butyldimethylsilyl)oxy)tetrahydro-2H-pyran-2-yl)methyl 4-methylbenzenesulfonate. A mixture consisting of (5-((tert-butyldimethylsilyl)oxy)tetrahydro-2H-pyran-2-yl)methanol (5.0 g, 20 mmol, 1 equiv.), DMAP (3.0 g, 24 mmol, 1.2 equiv.), and DCM (50 mL) was cooled to 0° C. and 4-toluenesulfonyl chloride (4.3 g, 22.3 mmol, 1.1 equiv.) was added. The reaction mixture was stirred overnight at room temperature. The reaction mixture was diluted with H₂O (100 mL), washed with 1 M HCl (50 mL) followed by saturated aqueous NaHCO₃ (50 mL), dried over sodium sulfate, filtered, and concentrated to dryness under reduced pressure to give the crude product, (5-((tert-butyldimethylsilyl)oxy)tetrahydro-2H-pyran-2-yl)methyl 4-methylbenzenesulfonate, as a colorless oil (6.5 g, 80%).

Step D. 5-(5-(((2*S,5*R)-5-((tert-butyldimethylsilyl)oxy)tetrahydro-2H-pyran-2-yl)methoxy)-2-methylpyridin-4-yl)-N-(2,6-dimethylpyrimidin-4-yl)pyrazolo[1,5-a]pyridin-2-amine. A mixture consisting of Intermediate 16 (500 mg, 1.4 mmol, 1 equiv.), (5-((tert-butyldimethylsilyl)oxy)tetrahydro-2H-pyran-2-yl)methyl 4-methylbenzenesulfonate (752 mg, 1.9 mmol, 1.3 equiv.), cesium carbonate (941 mg, 2.9 mmol, 2 equiv.), and DMF (10 mL) was heated via microwave at 90° C. for 1 hr. The reaction mixture was directly concentrated to afford the crude product, which was purified by FCC (eluent: petroleum ether:ethyl acetate=1:0 to 0:1) to afford the title compound, 5-(5-((5-((tert-butyldimethylsilyl)oxy)tetrahydro-2H-pyran-2-yl)methoxy)-2-methylpyridin-4-yl)-N-(2,6-dimethylpyrimidin-4-yl)pyrazolo[1,5-a]pyridin-2-amine, as a yellow solid. The material was further purified by SFC over DAICEL CHIRALPAK AD 250 mm×30 mm, 10 μm (eluent: 50% to 50% (v/v) supercritical CO₂ in MeOH and H₂O with 0.1% NH₃). The pure fractions were collected and the volatiles were removed under reduced pressure. The product was suspended in water (10 mL), the mixture frozen using dry ice/acetone, and then lyophilized to dryness to afford 5-(5-(42*S,5*R)-5-((tert-butyldimethylsilyl)oxy)tetrahydro-2H-pyran-2-yl)methoxy)-2-methylpyridin-4-yl)-N-(2,6-dimethylpyrimidin-4-yl)pyrazolo[1,5-a]pyridin-2-amine (50 mg).

Step E. (3*R,6*S)-6-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol. A solution consisting of 5-(5-(((2*S,5*R)-5-((tert-butyldimethylsilyl)oxy)tetrahydro-2H-pyran-2-yl)methoxy)-2-methylpyridin-4-yl)-N-(2,6-dimethylpyrimidin-4-yl)pyrazolo[1,5-a]pyridin-2-amine (120 mg, 0.21 mmol, 1 equiv.), trifluoroacetic acid (2 mL), and DCM (1 mL) was stirred for 1 hr at room temperature. The reaction mixture was directly concentrated under reduced pressure to afford crude product. Water (5 mL) was added to the residue and the pH was adjusted to 8 with 1 M NaHCO₃. A yellow precipitate was formed and collected by filtration then air-dried. The product was suspended in water (10 mL), the mixture frozen using dry ice/ethanol, and then lyophilized to dryness to afford the title compound, (3*R,6*S)-6-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol, as a yellow solid (97.3 mg, 81%). MS (ESI): mass calcd. for C₂₅H₂₈N₆O₃, 460.2. m/z found, 461.2 [M+H]⁺. ¹H NMR (400 MHz, DMSO-d6) δ 10.21 (s, 1H), 8.59 (d, J=6.8 Hz, 1H), 8.32 (s, 1H), 7.92 (s, 1H), 7.38 (s, 1H), 7.07 (d, J=6.4 Hz, 1H), 7.01 (s, 1H), 6.88 (s, 1H), 4.80 (d, J=4.8 Hz, 1H), 4.20-4.03 (m, 2H), 3.92-3.81 (m, 1H), 3.62-3.51 (m, 1H), 3.49-3.39 (m, 1H), 3.05-2.92 (m, 1H), 2.48-2.42 (m, 6H), 2.30 (s, 3H), 2.03-1.91 (m, 1H), 1.76-1.64 (m, 1H), 1.49-1.28 (m, 2H).

### Example 347: (3*S,6*S)-6-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol

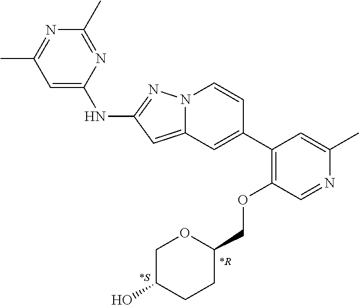

The title compound (62.3 mg, 52%) was prepared as described in Example 346, except 5-(5-(((2*R,5*S)-5-((tert-butyldimethylsilyl)oxy)tetrahydro-2H-pyran-2-yl)methoxy)-2-methylpyridin-4-yl)-N-(2,6-dimethylpyrimidin-4-yl)pyrazolo[1,5-a]pyridin-2-amine (1 equiv.) was used instead of 5-(5-(((2*S,5*R)-5-((tert-butyldimethylsilyl)oxy)tetrahydro-2H-pyran-2-yl)methoxy)-2-methylpyridin-4-yl)-N-(2,6-dimethylpyrimidin-4-yl)pyrazolo[1,5-a]pyridin-2-amine in Step E. MS (ESI): mass calcd. for C₂₅H₂₈N₆O₃, 460.2. m/z found, 461.2 [M+H]⁺. ¹H NMR (400 MHz, DMSO-d6) δ 10.23 (s, 1H), 8.60 (d, J=6.8 Hz, 1H), 8.32 (s, 1H), 7.93 (d, J=1.2 Hz, 1H), 7.38 (s, 1H), 7.08 (dd, J=2.0, 7.2 Hz, 1H), 7.01 (s, 1H), 6.88 (s, 1H), 4.81 (d, J=5.2 Hz, 1H), 4.18-4.04 (m, 2H), 3.92-3.81 (m, 1H), 3.61-3.50 (m, 1H), 3.49-3.39 (m, 1H), 3.04-2.94 (m, 1H), 2.48-2.43 (m, 6H), 2.31 (s, 3H), 2.03-1.91 (m, 1H), 1.76-1.64 (m, 1H), 1.48-1.26 (m, 2H)

### Example 348: N-(2,6-dimethylpyrimidin-4-yl)-6-[2-methyl-5-[[(1R,5S)-3-oxa-9-azabicyclo[3.3.1]nonan-7-yl]oxy]-4-pyridyl]imidazo[1,2-a]pyrazin-2-amine

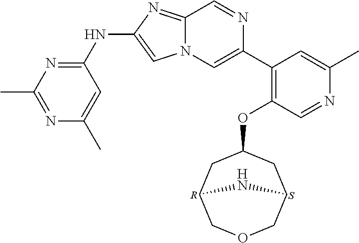

The title compound was prepared in the similar manner of Example 320 except 4-chloro-2,6-dimethylpyrimidine was used instead of 2-chloro-5-methylpyrazine in Step B. MS (ESI): mass calcd. for C₂₆H₂₉N₇O₂, 471.6. m/z found, 472.2 [M+H]⁺. ¹H NMR (500 MHz, Methanol-d4) δ 8.30 (d, J=7.2 Hz, 1H), 8.06 (s, 1H), 7.96 (d, J=1.8 Hz, 1H), 7.28 (s, 1H), 7.07 (dd, J=7.2, 2.0 Hz, 1H), 6.92 (s, 1H), 6.71 (s, 1H), 4.58-4.50 (m, 1H), 3.75-3.56 (m, 4H), 2.94-2.86 (m, 2H), 2.43-2.41 (two s, 6H), 2.34-2.24 (m, 5H), 1.92-1.82 (m, 2H)

### Example 349: N-(2,6-dimethylpyrimidin-4-yl)-6-[2-methyl-5-[[(1R,5S)-9-methyl-3-oxa-9-azabicyclo[3.3.1]nonan-7-yl]oxy]-4-pyridyl]imidazo[1,2-a]pyrazin-2-amine

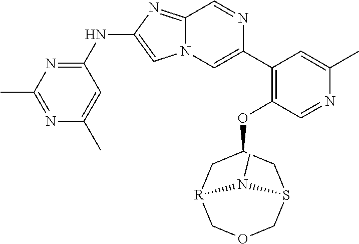

A mixture of N-(2,6-dimethylpyrimidin-4-yl)-6-[2-methyl-5-[[(1R,5S)-3-oxa-9-azabicyclo[3.3.1]nonan-7-yl]oxy]-4-pyridyl]imidazo[1,2-a]pyrazin-2-amine (Example 350) (64.0 mg, 0.109 mmol), TEA (22.1 mg, 0.218 mmol) and paraformaldehyde (10.8 mg, 0.120 mmol) in MeOH (2 mL) was stirred at 25° C. for 5 min. then NaBH₃CN (13.7 mg, 0.218 mmol) was added to the reaction mixture. The reaction mixture was stirred at 25° C. for 1 hours. The mixture was filtered, and the filtrate was purified by preparative HPLC using a Phenomenex Gemini-NX C18 75*30 mm*3 um (eluent: 22% to 55% (v/v) water (0.04% NH₃-H₂O+10 mM NH₄HCO₃)-ACN to afford pure product. The product was suspended in water (10 mL), the mixture frozen using dry ice/ethanol, and then lyophilized to dryness to afford the title compound as a white solid (24.5 mg, 45%). MS (ESI): mass calcd. for C₂₆H₃₀N₈O₂, 486.2. m/z found, 487.2 [M+H]⁺. ¹H NMR (400 MHz, CDCl₃) δ 10.04 (s, 1H), 8.95 (s, 1H), 8.37 (s, 1H), 8.18 (s, 1H), 8.01 (s, 1H), 7.62 (s, 1H), 6.52 (s, 1H), 4.91-4.84 (m, 1H), 4.20-4.10 (m, 2H), 3.93-3.87 (m, 2H), 2.84-2.75 (m, 2H), 2.67 (s, 3H), 2.62-2.52 (m, 8H), 2.43 (s, 3H), 1.92-1.89 (m, 1H), 1.88-1.84 (m, 1H).

### Example 351: (3*R, 5*S)-5-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol

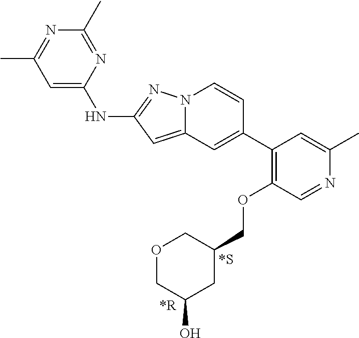

Step A. ((3S,5R)-5-((tert-butyldimethylsilyl)oxy)tetrahydro-2H-pyran-3-yl)methyl 4-methylbenzenesulfonate. A solution consisting of ((3R,5R)-5-((tert-butyldimethylsilyl)oxy)tetrahydro-2H-pyran-3-yl)methanol (700 mg, 2.8 mmol, 1 equiv.), DMAP (521 mg, 4.3 mmol, 1.5 equiv.), and dichloromethane (15 mL) was treated with TsCl (596 mg, 3.13 mmol, 1.1 equiv.) at room-temperature. The mixture was stirred overnight at room-temperature. The mixture was concentrated under reduced pressure to give a crude product which was purified by FCC (eluent: petroleum ether:CH₂Cl₂=1:0 to 3:1) to afford the product, ((3S,5R)-5-((tert-butyldimethylsilyl)oxy)tetrahydro-2H-pyran-3-yl)methyl 4-methylbenzenesulfonate, as a colorless oil (550 mg, 48%).

Step B. 5-(5-(((3S,5R)-5-((tert-butyl dimethyl silyl)oxy)tetrahydro-2H-pyran-3-yl)methoxy)-2-methylpyridin-4-yl)-N-(2,6-dimethylpyrimidin-4-yl)pyrazolo[1,5-a]pyridin-2-amine. To a mixture containing Intermediate 16 (290 mg, 0.84 mmol, 1 equiv.), cesium carbonate (818 mg, 2.5 mmol, 3 equiv.), and DMF (5 mL), was added ((3S,5R)-5-((tert-butyldimethylsilyl)oxy)tetrahydro-2H-pyran-3-yl)methyl 4-methylbenzenesulfonate (503 mg, 0.84 mmol, 1.5 equiv.) at room temperature. The mixture was stirred for 1 hour at 90° C. via microwave. The mixture was concentrated under reduced pressure to give a crude product which was purified by FCC (eluent: petroleum ether: (ethyl acetate:EtOH=3:1)=1:0 to 1:1) to afford 5-(5-(((3S,5R)-5-((tert-butyldimethylsilyl)oxy)tetrahydro-2H-pyran-3-yl)methoxy)-2-methylpyridin-4-yl)-N-(2,6-dimethylpyrimidin-4-yl)pyrazolo[1,5-a]pyridin-2-amine as a yellow solid (170 mg, 32%).

Step C. (3S,5R)-5-(((4-(2-((2,6-dimethylpyrimidin-4-yl)amino)pyrazolo[1,5-a]pyridin-5-yl)-6-methylpyridin-3-yl)oxy)methyl)tetrahydro-2H-pyran-3-ol. A mixture consisting of 5-(5-(((3S,5R)-5-((tert-butyldimethylsilyl)oxy)tetrahydro-2H-pyran-3-yl)methoxy)-2-methylpyridin-4-yl)-N-(2,6-dimethylpyrimidin-4-yl)pyrazolo[1,5-a]pyridin-2-amine (150 mg, 0.261 mmol) and TFA/CH₂Cl₂ (5 mL, ⅓) was stirred at room temperature for 1 hr. The solution was directly concentrated to afford the crude product, which was purified by preparative HPLC using a Phenomenex Gemini-NX 150×30 mm×5 μm column (eluent: 21% to 51% (v/v) CH₃CN with 0.05% NH₃—H₂O) to afford pure product. The product was suspended in water (10 mL), the mixture frozen using dry ice/ethanol, and then lyophilized to dryness to afford (3S,5R)-5-(((4-(2-(2,6-dimethylpyrimidin-4-yl)amino)pyrazolo[1,5-a]pyridin-5-yl)-6-methylpyridin-3-yl)oxy)methyl)tetrahydro-2H-pyran-3-ol as a white solid (90 mg, 74%).

Step D. (3*R, 5*S)-5-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol. The material (3S,5R)-5-(((4-(2-((2,6-dimethylpyrimidin-4-yl)amino)pyrazolo[1,5-a]pyridin-5-yl)-6-methylpyridin-3-yl)oxy)methyl)tetrahydro-2H-pyran-3-ol (90 mg, 0.055 mmol) was further purified by SFC with a Daicel ChiralPak AD (250 mm×30 mm, 10 um) column (eluent: 55% to 55% (v/v) EtOH with 0.1% NH₃—H₂O in supercritical CO₂) to afford pure product. The product was suspended in water (10 mL), the mixture frozen using dry ice/EtOH, and then lyophilized to dryness to afford the title compound (3*R, 5*S)-5-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol as a white solid (36.8 mg, 40%). MS (ESI): mass calcd. for C₂₅H₂₈N₆O₃, 460.2. m/z found, 461.2 [M+H]⁺. ¹H NMR (400 MHz, CDCl₃) δ 8.38 (d, J=7.2 Hz, 1H), 8.23 (s, 1H), 7.61-7.54 (m, 2H), 7.17 (s, 1H), 7.10 (s, 1H), 6.90 (dd, J=1.6, 7.2 Hz, 1H), 6.57 (s, 1H), 4.04-3.97 (m, 2H), 3.97-3.88 (m, 2H), 3.84-3.75 (m, 1H), 3.26-3.16 (m, 1H), 3.13-3.03 (m, 1H), 2.61 (s, 3H), 2.56 (s, 3H), 2.45 (s, 3H), 2.28-2.11 (m, 2H), 2.04 (br. S., 1H), 1.40-1.29 (m, 1H).

### Example 352: (rac-)-N-(2,6-dimethylpyrimidin-4-yl)-5-[5-[(1,1-dioxo-1,2,5-thiadiazolidin-3-yl)methoxy]-2-methyl-4-pyridyl]pyrazolo[1,5-a]pyridin-2-amine

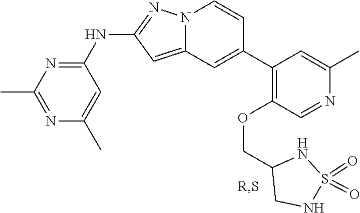

Step A. 2,2,12,12-tetramethyl-4,10-dioxo-3,11-dioxa-5,9-diazatridecan-7-yl 4-methylbenzenesulfonate. A solution consisting of di-tert-butyl (2-hydroxypropane-1,3-diyl)dicarbamate (600 mg, 2.1 mmol, 1 equiv.), DMAP (379 mg, 3.1 mmol, 1.5 equiv.), and dichloromethane (15 mL) was treated with TsCl (433 mg, 2.3 mmol, 1.1 equiv.) at room-temperature. The mixture was stirred for 3 hr at room-temperature. The mixture was concentrated under reduced pressure to give a crude product which was purified by FCC (eluent: petroleum ether:CH₂Cl₂=1:0 to 3:1) to afford the product, 2,2,12,12-tetramethyl-4,10-dioxo-3,11-dioxa-5,9-diazatridecan-7-yl 4-methylbenzenesulfonate, as a colorless oil (850 mg, 93%).

Step B. Di-tert-butyl (2-((4-(2-((2,6-dimethylpyrimidin-4-yl)amino)pyrazolo[1,5-a]pyridin-5-yl)-6-methylpyridin-3-yl)oxy)propane-1,3-diyl)dicarbamate. Di-tert-butyl (3-((4-(2-((2,6-dimethylpyrimidin-4-yl)amino)pyrazolo[1,5-a]pyridin-5-yl)-6-methylpyridin-3-yl)oxy)propane-1,2-diyl)dicarbamate. A mixture consisting of Intermediate 16 (500 mg, 1.44 mmol, 1 equiv.), 2,2,12,12-tetramethyl-4,10-dioxo-3,11-dioxa-5,9-diazatridecan-7-yl 4-methylbenzenesulfonate (834 mg, 1.88 mmol, 1.3 equiv.), cesium carbonate (941 mg, 2.89 mmol, 2 equiv.),potassium iodide (240 mg, 1.44 mmol, 1 equiv.) and DMF (10 mL) was stirred for 2 hr at 120° C. The mixture was concentrated under reduced pressure to give a crude product which was purified by FCC (eluent: petroleum ether: (ethyl acetate:EtOH=3:1)=1:0 to 1:1) to afford a mixture of products, di-tert-butyl (2-((4-(2-((2,6-dimethylpyrimidin-4-yl)amino)pyrazolo[1,5-a]pyridin-5-yl)-6-methylpyridin-3-yl)oxy)propane-1,3-diyl)dicarbamate and di-tert-butyl (3-((4-(2-((2,6-dimethylpyrimidin-4-yl)amino)pyrazolo[1,5-a]pyridin-5-yl)-6-methylpyridin-3-yl)oxy)propane-1,2-diyl)dicarbamate as a yellow solid (400 mg, 21%).

Step C. 2-((4-(2-((2,6-dimethylpyrimidin-4-yl)amino)pyrazolo[1,5-a]pyridin-5-yl)-6-methylpyridin-3-yl)oxy)propane-1,3-diamine. 3-((4-(2-((2,6-dimethylpyrimidin-4-yl)amino)pyrazolo[1,5-a]pyridin-5-yl)-6-methylpyridin-3-yl)oxy)propane-1,2-diamine. A solution consisting of a mixture of di-tert-butyl (2-((4-(2-((2,6-dimethylpyrimidin-4-yl)amino)pyrazolo[1,5-a]pyridin-5-yl)-6-methylpyridin-3-yl)oxy)propane-1,3-diyl)dicarbamate and di-tert-butyl (3-((4-(2-((2,6-dimethylpyrimidin-4-yl)amino)pyrazolo[1,5-a]pyridin-5-yl)-6-methylpyridin-3-yl)oxy)propane-1,2-diyl)dicarbamate (400 mg, 0.32 mmol, 1 equiv.) and trifluoroacetic acid/DCM (10 mL, 1:3) was stirred at room temperature for 1 hr. The mixture was concentrated under reduced pressure to give a crude product. The product was suspended in water (10 mL), the mixture frozen using dry ice/EtOH, and then lyophilized to dryness to afford a mixture of compounds 2-((4-(2-((2,6-dimethylpyrimidin-4-yl)amino)pyrazolo[1,5-a]pyridin-5-yl)-6-methylpyridin-3-yl)oxy)propane-1,3-diamine and 3-((4-(2-((2,6-dimethylpyrimidin-4-yl)amino)pyrazolo[1,5-a]pyridin-5-yl)-6-methylpyridin-3-yl)oxy)propane-1,2-diamine as a yellow solid (400 mg, 139%).

Step D. (RS)—N-(2,6-dimethylpyrimidin-4-yl)-5-[5-[(1,1-dioxo-1,2,5-thiadiazolidin-3-yl)methoxy]-2-methyl-4-pyridyl]pyrazolo[1,5-a]pyridin-2-amine. A mixture consisting of 2-((4-(2-((2,6-dimethylpyrimidin-4-yl)amino)pyrazolo[1,5-a]pyridin-5-yl)-6-methylpyridin-3-yl)oxy)propane-1,3-diamine and 3-((4-(2-((2,6-dimethylpyrimidin-4-yl)amino)pyrazolo[1,5-a]pyridin-5-yl)-6-methylpyridin-3-yl)oxy)propane-1,2-diamine (380 mg, 0.908 mmol, 1 equiv.), sulfuric diamide (436 mg, 4.54 mmol, 5 equiv.), and Py (10 mL) was stirred at 120° C. via microwave for 20 min. The solution was concentrated to afford the crude product, which was purified by preparative HPLC using a Phenomenex Gemini-NX 150×30 mm×5 μm column (eluent: 25% to 55% (v/v) CH₃CN with 0.05% NH₃—H₂O) to afford pure product. The product was suspended in water (10 mL), the mixture frozen using dry ice/ethanol, and then lyophilized to dryness to afford the product, (RS)—N-(2,6-dimethylpyrimidin-4-yl)-5-[5-[(1,1-dioxo-1,2,5-thiadiazolidin-3-yl)methoxy]-2-methyl-4-pyridyl]pyrazolo[1,5-a]pyridin-2-amine, as an off-white solid (12.5 mg, 5%). MS (ESI): mass calcd. for C₂₂H₂₄N₈O₃S, 480.2. m/z found, 481.1 [M+H]+. 1H NMR (400 MHz, DMSO-d6) δ 10.22 (s, 1H), 8.56 (d, J=7.2 Hz, 1H), 8.35 (s, 1H), 7.86 (s, 1H), 7.38 (s, 1H), 7.28 (d, J=4.8 Hz, 1H), 7.07 (dd, J=1.6, 7.2 Hz, 1H), 7.02 (br. s., 1H), 6.95 (t, J=7.2 Hz, 1H), 6.89 (s, 1H), 4.14 (d, J=6.4 Hz, 2H), 4.04-3.91 (m, 1H), 3.46-3.43 (m, 1H), 3.22-3.12 (m, 1H), 2.47 (s, 3H), 2.46 (s, 3H), 2.30 (s, 3H).

### Example 353: (rac-)-N-(2,6-dimethylpyrimidin-4-yl)-5-[5-[(1,1-dioxo-1,2-thiazolidin-3-yl)methoxy]-2-methyl-4-pyridyl]pyrazolo[1,5-a]pyridin-2-amine

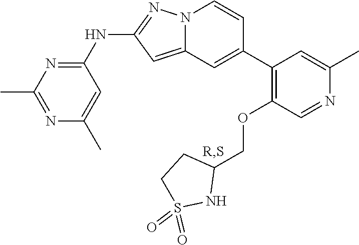

To a mixture consisting of Intermediate 16 (250 mg, 0.722 mmol, 1 equiv.), and TEA (219 mg, 2.17 mmol, 3 equiv.) in EtOH (5 mL) was added 2-thia-1-azabicyclo[3.1.0]hexane 2,2-dioxide (96.1 mg, 0.72 mmol, 1 equiv.). The mixture was stirred overnight at 60° C. The solution was concentrated to afford the crude product, which was purified by preparative HPLC using a Phenomenex Gemini-NX 150×30 mm×5 μm column (eluent: 22% to 52% (v/v) CH₃CN with 0.05% NH₃—H₂O) to afford product. The product was suspended in water (10 mL) then the mixture was frozen using dry ice/ethanol then lyophilized to dryness to afford the crude product as an off-white solid (15 mg, 4%). The material was further purified by SFC with a DAICEL CHIRALCEL OJ (250 mm×50 mm, 10 um) column (eluent: 50% to 50% (v/v) EtOH with 0.1% NH₃—H₂O in supercritical CO₂) to afford pure product. The product was suspended in water (10 mL), the mixture frozen using dry ice/EtOH, and then lyophilized to dryness to afford (RS)—N-(2,6-dimethylpyrimidin-4-yl)-5-[5-[(1,1-dioxo-1,2-thiazolidin-3-yl)methoxy]-2-methyl-4-pyridyl]pyrazolo[1,5-a]pyridin-2-amine as an off-white solid (12.6 mg, 88%). MS (ESI): mass calcd. for C₂₃H₂₅N₇O₃S, 479.2. m/z found, 480.1 [M+H]⁺. ¹H NMR (400 MHz, DMSO-d6) δ 10.22 (s, 1H), 8.55 (d, J=7.2 Hz, 1H), 8.35 (s, 1H), 7.88 (s, 1H), 7.38 (s, 1H), 7.30 (d, J=4.8 Hz, 1H), 7.09 (dd, J=1.6, 7.2 Hz, 1H), 7.02 (br. s., 1H), 6.90 (br. s., 1H), 4.11 (d, J=5.2 Hz, 2H), 3.92-3.80 (m, 1H), 3.20-3.09 (m, 1H), 3.06-2.95 (m, 1H), 2.47 (s, 3H), 2.46 (s, 3H), 2.45-2.41 (m, 1H), 2.30 (s, 3H), 2.15-2.02 (m, 1H).

### Example 354: (*R)—N-(2,6-dimethylpyrimidin-4-yl)-5-[2-methyl-5-(2-methyl-1,1-dioxo-thiazinan-4-yl)oxy-4-pyridyl]pyrazolo[1,5-a]pyridin-2-amine

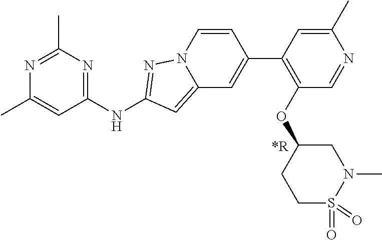

Step A. 4-((4-(2-((2,6-dimethylpyrimidin-4-yl)amino)pyrazolo[1,5-a]pyridin-5-yl)-6-methylpyridin-3-yl)oxy)-1,2-thiazinane 1,1-dioxide. To a mixture consisting of Intermediate 16 (4-(2-((2,6-dimethylpyrimidin-4-yl)amino)pyrazolo[1,5-a]pyridin-5-yl)-6-methylpyridin-3-ol) (250 mg, 0.722 mmol, 1 equiv.), TEA (219 mg, 2.17 mmol, 3 equiv.) and EtOH (5 mL) was added 2-thia-1-azabicyclo[3.1.0]hexane 2,2-dioxide (96.1 mg, 0.722 mmol, 1 equiv.). The mixture was stirred for 2 days at 60° C. The solution was concentrated to afford the title product (400 mg, crude) as a yellow solid.

Step B. (*R)—N-(2,6-dimethylpyrimidin-4-yl)-5-[2-methyl-5-(2-methyl-1,1-dioxo-thiazinan-4-yl)oxy-4-pyridyl]pyrazolo[1,5-a]pyridin-2-amine. The material (trimethylsilyl)diazomethane (1.10 mL, 2.19 mmol, 3 equiv., 2M in hexane) was added dropwise to a solution consisting of 4-((4-(2-((2,6-dimethylpyrimidin-4-yl)amino)pyrazolo[1,5-a]pyridin-5-yl)-6-methylpyridin-3-yl)oxy)-1,2-thiazinane 1,1-dioxide (350 mg, 0.73 mmol, 1 equiv.) and MeOH/DCM (15 mL, 1:2). The resultant mixture was stirred overnight at 30° C. The solution was concentrated to afford the crude product, which was purified by preparative HPLC using a Boston Prime C18 150×30 mm×5 μm column (eluent: 30% to 60% (v/v) CH₃CN with 0.05% NH₃-H₂O+10 mM NH₄HCO₃) to afford pure product. The product was suspended in water (10 mL), the mixture frozen using dry ice/ethanol, and then lyophilized to dryness to afford (RS)—N-(2,6-dimethylpyrimidin-4-yl)-5-[2-methyl-5-(2-methyl-1,1-dioxo-thiazinan-4-yl)oxy-4-pyridyl]pyrazolo[1,5-a]pyridin-2-amine (60 mg, 16%) as a yellow solid. The material was further purified by SFC with a DAICEL CHIRALPAK AD (250 mm×30 mm, 10 um) column (eluent: 50% to 50% (v/v) IPA with 0.1% NH₃—H₂O in supercritical CO₂) to afford pure product. The first eluted isomer was designated as *R isomer. The product was suspended in water (10 mL), the mixture frozen using dry ice/EtOH, and then lyophilized to dryness to afford the title compound (13.5 mg, 22%) as a white solid. MS (ESI): mass calcd. for C₂₄H₂₇N₇O₃S, 493.2. m/z found, 494.1 [M+H]⁺. ¹H NMR (400 MHz, CDCl₃) δ 8.41 (d, J=7.2 Hz, 1H), 8.28 (s, 1H), 7.68 (br. s., 1H), 7.64 (s, 1H), 7.23 (s, 1H), 7.12 (s, 1H), 6.93 (dd, J=2.0, 7.6 Hz, 1H), 6.63 (s, 1H), 4.49-4.42 (m, 1H), 3.60-3.53 (m, 1H), 3.45-3.38 (m, 1H), 3.29-3.18 (m, 1H), 3.02-2.94 (m, 1H), 2.73 (s, 3H), 2.61 (s, 3H), 2.59 (s, 3H), 2.51-2.43 (m, 5H)

### Example 355: (*S)—N-(2,6-dimethylpyrimidin-4-yl)-5-[2-methyl-5-(2-methyl-1,1-dioxo-thiazinan-4-yl)oxy-4-pyridyl]pyrazolo[1,5-a]pyridin-2-amine

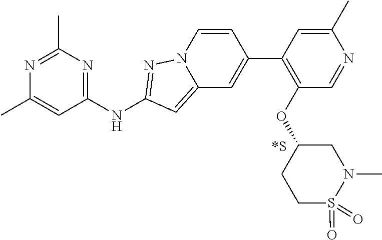

The title compound (12.8 mg, 21%) was prepared as described in Example 354, except the second elute isomer by SFC with a DAICEL CHIRALPAK AD was designated as *S isomer. MS (ESI): mass calcd. for C₂₄H₂₇N₇O₃S, 493.2. m/z found, 494.1 [M+H]⁺. ¹H NMR (400 MHz, CDCl₃) δ 8.41 (d, J=7.2 Hz, 1H), 8.28 (s, 1H), 7.67 (br. s., 1H), 7.63 (s, 1H), 7.23 (s, 1H), 7.11 (s, 1H), 6.93 (dd, J=2.0, 7.2 Hz, 1H), 6.63 (s, 1H), 4.50-4.40 (m, 1H), 3.61-3.51 (m, 1H), 3.45-3.37 (m, 1H), 3.29-3.18 (m, 1H), 3.03-2.93 (m, 1H), 2.73 (s, 3H), 2.61 (s, 3H), 2.59 (s, 3H), 2.53-2.39 (m, 5H)

### Example 356: 6-[[5-[5-[[(1S,5R)-9-acetyl-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide

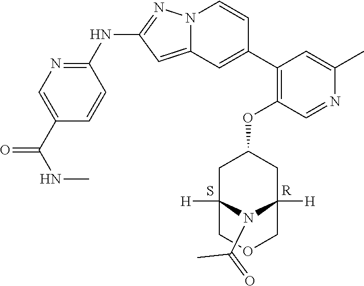

Step A. 5-(5-(((1R,5S,7s)-3-oxa-9-azabicyclo[3.3.1]nonan-7-yl)oxy)-2-methylpyridin-4-yl)pyrazolo[1,5-a]pyridin-2-amine. A mixture consisting of

Intermediate 9 (900 mg, 0.86 mmol, 1 equiv.) and trifluoroacetic acid/DCM (20 mL, 1:3) was stirred at room temperature for 2 hr. The mixture was concentrated under reduced pressure to give a crude product. The product was suspended in water (10 mL), the mixture frozen using dry ice/EtOH, and then lyophilized to dryness to afford 5-(5-(((1R,5S,7s)-3-oxa-9-azabicyclo[3.3.1]nonan-7-yl)oxy)-2-methylpyridin-4-yl)pyrazolo[1,5-a]pyridin-2-amine (500 mg, crude) as a yellow solid.

Step B. 1-((1R,5S,7s)-7-((4-(2-aminopyrazolo[1,5-a]pyridin-5-yl)-6-methylpyridin-3-yl)oxy)-3-oxa-9-azabicyclo[3.3.1]nonan-9-yl)ethan-1-one. To a mixture consisting of 5-(5-(((1R,5S,7s)-3-oxa-9-azabicyclo[3.3.1]nonan-7-yl)oxy)-2-methylpyridin-4-yl)pyrazolo[1,5-a]pyridin-2-amine (440 mg, 1.20 mmol, 1 equiv.), TEA, (366 mg, 3.61 mmol, 3 equiv.) and DCM (10 mL) at 0° C., acetyl chloride (70.9 mg, 0.90 mmol, 0.75 equiv.) in DCM (1 mL) was added dropwise. The mixture was stirred at 0° C. for 0.5 hr. The mixture was concentrated under reduced pressure to give the product, 1-41R,5S,7s)-7-((4-(2-aminopyrazolo[1,5-a]pyridin-5-yl)-6-methylpyridin-3-yl)oxy)-3-oxa-9-azabicyclo[3.3.1]nonan-9-yl)ethan-1-one (500 mg, crude), as a yellow solid.

Step C. 6-[[5-[5-[[(1S,5R)-9-acetyl-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide. A mixture consisting 1-((1R,5S,7s)-7-((4-(2-aminopyrazolo[1,5-a]pyridin-5-yl)-6-methylpyridin-3-yl)oxy)-3-oxa-9-azabicyclo[3.3.1]nonan-9-yl)ethan-1-one (92.1 mg, 0.540 mmol, 1 equiv.), 6-chloro-N-methylnicotinamide (92.1 mg, 0.54 mmol, 1 equiv.), Brettphos-Pd-G3 (49 mg, 0.054 mmol. 0.1 equiv.), Cs₂CO₃ (528 mg, 1.62 mmol, 3 equiv.), and 1,4-dioxane (7 mL) was stirred for 3 h at 120° C. under N₂. The solution was concentrated to afford the crude product, which was purified by preparative HPLC using a Boston Prime C18 150×30 mm×5 μm column (eluent: 25% to 55% (v/v) CH₃CN and H₂O with 0.05% NH₃-H₂O+10 mM NH₄HCO₃) to afford pure product. The product was suspended in water (10 mL), the mixture frozen using dry ice/ethanol, and then lyophilized to dryness to afford the title compound, 6-[[5-[5-[[(1S,5R)-9-acetyl-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide, as a yellow solid (47.6 mg, 16%). MS (ESI): mass calcd. for C₂₉H₃₁N₇O₄, 541.2. m/z found, 542.2 [M+H]⁺. ¹H NMR (400 MHz, DMSO-d6) δ 10.21 (s, 1H), 8.71 (d, J=2.0 Hz, 1H), 8.55 (d, J=7.2 Hz, 1H), 8.37-8.31 (m, 1H), 8.26 (s, 1H), 8.07 (d, J=1.2 Hz, 1H), 8.04 (dd, J=2.0, 8.4 Hz, 1H), 7.39 (s, 1H), 7.35 (d, J=8.4 Hz, 1H), 7.15 (dd, J=1.6, 6.8 Hz, 1H), 6.86 (s, 1H), 4.52-4.41 (m, 2H), 4.03-3.96 (m, 1H), 3.75-3.64 (m, 2H), 3.61-3.53 (m, 1H), 3.48-3.42 (m, 1H), 2.79 (d, J=4.4 Hz, 3H), 2.47 (s, 3H), 2.44-2.35 (m, 1H), 2.26-2.15 (m, 1H), 2.01 (s, 3H), 1.92-1.81 (m, 2H)

### Example 357: 2,6-dimethoxy-N-methyl-4-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]benzamide

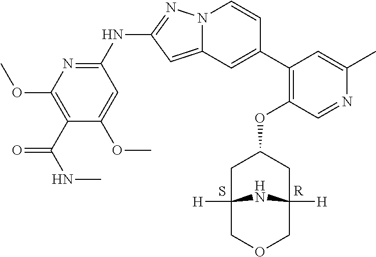

The title compound (60.1 mg, 62%) was prepared as described in Example 340, except 4-bromo-2,6-dimethoxy-N-methylbenzamide (1.5 equiv.) was used instead of 4-bromo-N-isopropyl-2,6-dimethoxybenzamide. The preparation of 4-bromo-2,6-dimethoxy-N-methylbenzamide was similar to Step A in Example 245, except propan-2-amine (2 equiv.) was used instead of cyclopropyl amine, DMF (5 mL) was used instead of DCM, and DIEA (3 equiv.) was use instead Et₃N. MS (ESI): mass calcd. for C₃₀H₃₄N₆O5, 558.3. m/z found, 559.2 [M+H]⁺. ¹H NMR (400 MHz, DMSO-d6) δ 9.12 (s, 1H), 8.53 (d, J=7.6 Hz, 1H), 8.28 (s, 1H), 7.93 (d, J=0.8 Hz, 1H), 7.79 (q, J=4.4 Hz, 1H), 7.37 (s, 1H), 7.06 (dd, J=2.0, 7.2 Hz, 1H), 6.87 (s, 2H), 6.12 (s, 1H), 4.70-4.62 (m, 1H), 3.73 (s, 6H), 3.60-3.56 (m, 2H), 3.55-3.50 (m, 2H), 2.95 (d, J=8.0 Hz, 2H), 2.66 (d, J=4.8 Hz, 3H), 2.46 (s, 3H), 2.34-2.25 (m, 2H), 1.72-1.64 (m, 2H).

### Example 358: 6-[[5-[5-[[(1R,5S,7s)-9-acetyl-3-oxa-9-azabicyclo[3.3.1]nonan-7-yl]oxy]-2-methyl-4-pyridyl]pyrazolo[1,5-a]pyridin-2-yl]amino]-N,2,4-trimethyl-pyridine-3-carboxamide

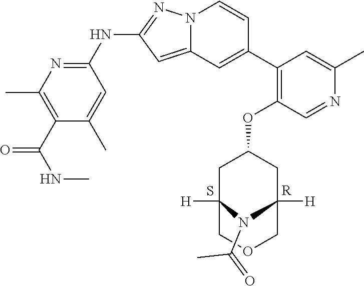

Step A. 6-chloro-2,4-dimethylnicotinic acid. To a solution consisting of methyl 6-chloro-2,4-dimethylnicotinate (500 mg, 2.51 mmol, 1 equiv.), H₂O (2 mL), MeOH (4 mL) and THF (4 mL) was added lithium hydroxide monohydrate (210 mg, 5.01 mmol, 2 equiv). The mixture was stirred at room-temperature for 1 hr. To the mixture was added additional lithium hydroxide monohydrate (315 mg, 7.51 mmol, 3 equiv.). The mixture was stirred at room-temperature for 1 hour. Under reduced pressure THF and MeOH was removed. The residue was acidified with aqueous citric acid solution to pH=6˜7. The reaction mixture was diluted with H₂O (10 mL) and extracted with EtOAc (30 mL×3). The organic phases were combined, dried over anhydrous sodium sulfate, filtered, and concentrated under reduced pressure to afford the product, 6-chloro-2,4-dimethylnicotinic acid, as a white solid (330 mg, 71%).

Step B. 6-chloro-N,2,4-trimethylnicotinamide. To a mixture of 6-chloro-2,4-dimethylnicotinic acid (280 mg, 1.51 mmol, 1 equiv.), MeNH₂HCl (204 mg, 3.02 mmol, 2 equiv.), DIEA (780 mg, 6.03 mmol, 4 equiv.) and DMF (5.5 mL) was added HATU (688 mg, 1.81 mmol, 1.2 equiv.). The mixture was stirred at room temperature for 1 hr. The reaction mixture was diluted with H₂O (10 mL) and extracted with EtOAc (20 mL×3). The organic phases were combined and concentrated under reduced pressure to afford the crude product, which was purified by FCC (ethyl acetate:petroleum ether=0 to 60%) to afford the compound, 6-chloro-N,2,4-trimethylnicotinamide, as a white solid (250 mg, 83%).

Step C. 6-[[5-[5-[[(1S,5R)-9-acetyl-3-oxa-9-azabicyclo[3.3.1]nonan-7-yl]oxy]-2-methyl-4-pyridyl]pyrazolo[1,5-a]pyridin-2-yl]amino]-N,2,4-trimethyl-pyridine-3-carboxamide. The title compound (58.6 mg, 23%), 6-[[5-[5-[[(1S,5R)-9-acetyl-3-oxa-9-azabicyclo[3.3.1]nonan-7-yl]oxy]-2-methyl-4-pyridyl]pyrazolo[1,5-a]pyridin-2-yl]amino]-N,2,4-trimethyl-pyridine-3-carboxamide, was prepared as described in Example 356, except 6-chloro-N,2,4-trimethylnicotinamide (1 equiv.) was used instead of 6-chloro-N-methylnicotinamide, Xantphos (0.2 equiv.) was used instead of Brettphos-Pd-G3, cesium fluoride (3 equiv.) was used instead of Cs₂CO₃, and palladium (II) acetate was added (0.1 equiv.). MS (ESI): mass calcd. for C₃₁H₃₅N₇O₄, 569.3. m/z found, 570.2 [M+H]⁺. ¹H NMR (400 MHz, DMSO-d₆) δ 9.75 (s, 1H), 8.51 (d, J=7.2 Hz, 1H), 8.25 (s, 1H), 8.18 (q, J=4.4 Hz, 1H), 8.04 (d, J=1.2 Hz, 1H), 7.40 (s, 1H), 7.12 (dd, J=2.0, 7.2 Hz, 1H), 7.05 (s, 1H), 6.81 (s, 1H), 4.50-4.43 (m, 2H), 4.00 (d, J=6.8 Hz, 1H), 3.73-3.67 (m, 2H), 3.57 (dd, J=2.0, 11.2 Hz, 1H), 3.45 (dd, J=2.8, 11.6 Hz, 1H), 2.76 (d, J=4.8 Hz, 3H), 2.46 (s, 3H), 2.44-2.36 (m, 1H), 2.33 (s, 3H), 2.27-2.19 (m, 1H), 2.17 (s, 3H), 2.01 (s, 3H), 1.91-1.81 (m, 2H).

### Example 359: 5-[5-(2-amino-2-methyl-propoxy)-2-methyl-4-pyridyl]-N-(2,6-dimethylpyrimidin-4-yl)pyrazolo[1,5-a]pyridin-2-amine

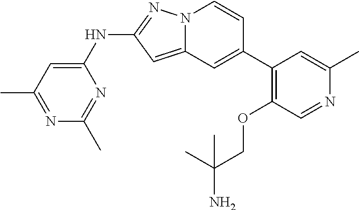

Step A. 2-methyl-2-nitropropyl 4-methylbenzenesulfonate. To a solution consisting of 2-methyl-2-nitropropan-1-ol (1.0 g, 8.4 mmol), DMAP (2.56 g, 21.0 mmol), and dichloromethane (20 mL) at 0° C. was added 4-toluenesulfonyl chloride (2.40 g, 12.6 mmol, 1.5 equiv.). The resultant mixture was stirred overnight at room-temperature. To the reaction mixture was added HCl (1 M, 20 mL) and DCM (20 mL) and the resultant mixture was stirred at room-temperature for 2 mins. The layers were separated and the reaction was extracted with dichloromethane to get the crude product, 2-methyl-2-nitropropyl 4-methylbenzenesulfonate (2.25 g, 98%).

Step B. N-(2,6-dimethylpyrimidin-4-yl)-5-(2-methyl-5-(2-methyl-2-nitropropoxy)pyridin-4-yl)pyrazolo[1,5-a]pyridin-2-amine. To a mixture consisting of Intermediate 16 (400 mg, 1.16 mmol, 1 equiv.), 2-methyl-2-nitropropyl 4-methylbenzenesulfonate (316 mg, 1.16 mmol), and DMF (5 mL) was added Cs₂CO₃ (753 mg, 2.31 mmol, 2 equiv.) and the resultant mixture was heated to 80° C. via micro'wave irradiation for 1 hour before cooling to room-temperature. The reaction mixture was heated to 80° C. via microwave irradiation for 6 hr resulting in the crude material, N-(2,6-dimethylpyrimidin-4-yl)-5-(2-methyl-5-(2-methyl-2-nitropropoxy)pyridin-4-yl)pyrazolo[1,5-a]pyridin-2-amine (230 mg, 45%).

Step C. 5-[5-(2-amino-2-methyl-propoxy)-2-methyl-4-pyridyl]-N-(2,6-dimethylpyrimidin-4-yl)pyrazolo[1,5-a]pyridin-2-amine. A mixture consisting of N-(2,6-dimethylpyrimidin-4-yl)-5-(2-methyl-5-(2-methyl-2-nitropropoxy)pyridin-4-yl)pyrazolo[1,5-a]pyridin-2-amine (220 mg, 0.492 mmol, 1 equiv.), MeOH (20 mL) and Pd/C (192 mg, 0.0980 mmol, 10%) was stirred at room temperature under H₂ (45 psi) overnight. The reaction mixture was continued to heat at 35° C. and stirred under H₂ (45 psi) overnight. The suspension was filtered through a pad of Celite® and the pad washed with MeOH (50 mL). The combined organic layers were concentrated to dryness under reduced pressure to give the crude product (170 mg). Of the crude product, 80 mg was purified by preparative HPLC BH using a Phenomenex Gemini-NX 80×40 mm×3 μm column (eluent: 25% to 49% (v/v) CH₃CN and H₂O with 0.05% NH₃-H₂O+10 mM NH₄HCO₃) to afford product. The product was suspended in water (10 mL), the mixture frozen using dry ice/ethanol, and then lyophilized to dryness to afford the title compound as a white solid (29.2 mg, 14%).MS (ESI): mass calcd. for C₂₃H₂₇N₇O, 417.2. m/z found, 418.2 [M+H]⁺. ¹H NMR (400 MHz, CDCl₃) δ 8.38 (d, J=7.6 Hz, 1H), 8.25 (s, 1H), 7.62-7.58 (m, 1H), 7.43 (br. s., 1H), 7.17 (s, 1H), 7.10 (s, 1H), 6.95 (dd, J=1.6, 7.2 Hz, 1H), 6.57 (s, 1H), 3.82 (s, 2H), 2.61 (s, 3H), 2.56 (s, 3H), 2.46 (s, 3H), 1.17 (s, 6H).

### Example 360: N-[2-[[4-[2-[(2,6-dimethylpyrimidin-4-yl)amino]pyrazolo[1,5-a]pyridin-5-yl]-6-methyl-3-pyridyl]oxy]-1,1-dimethyl-ethyl]acetamide

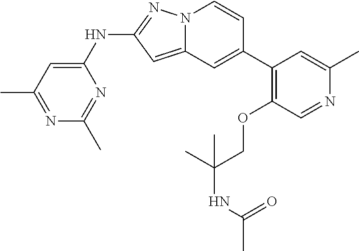

To a mixture 5-[5-(2-amino-2-methyl-propoxy)-2-methyl-4-pyridyl]-N-(2,6-dimethylpyrimidin-4-yl)pyrazolo[1,5-a]pyridin-2-amine (Example 359) (80 mg, 0.19 mmol, 1 equiv.), TEA (39 mg, 0.38 mmol), and DCM (2 mL), acetyl chloride (22.6 mg, 0.287 mmol, 1.5 equiv.) was added dropwise at 0° C. stirred for 1 hr. The reaction mixture was quenched with slow addition of MeOH (2 mL). The reaction mixture was concentrated under reduced pressure to afford the crude product, which was purified by preparative HPLC BH using a Phenomenex Gemini-NX C18 80×40 mm×3 μm column (eluent: 24% to 46% (v/v) water (0.05% NH₃—H₂O+10 mM NH₄HCO₃)-ACN) to afford pure product, which was suspended in water (10 mL), the mixture frozen using dry ice/ethanol, and then lyophilized to dryness to afford the title compound as a white solid (27.2 mg, 31%). MS (ESI): mass calcd. for C₂₅H₂₉N₇O₂, 459.2. m/z found, 460.2 [M+H]⁺. ¹H NMR (400 MHz, CDCl₃) δ 8.37 (d, J=7.6 Hz, 1H), 8.26 (s, 1H), 7.59 (d, J=0.8 Hz, 1H), 7.47 (s, 1H), 7.15 (s, 1H), 7.07 (s, 1H), 6.91 (dd, J=2.0, 7.2 Hz, 1H), 6.59 (s, 1H), 5.26 (s, 1H), 4.20 (s, 2H), 2.61 (s, 3H), 2.56 (s, 3H), 2.46 (s, 3H), 1.81 (s, 3H), 1.36 (s, 6H).

### Example 361: N-[2-[[4-[2-[(2,6-dimethylpyrimidin-4-yl)amino]pyrazolo[1,5-a]pyridin-5-yl]-6-methyl-3-pyridyl]oxy]-1,1-dimethyl-ethyl]methanesulfonamide

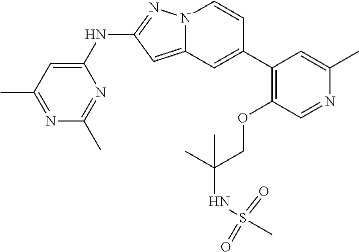

The title compound (40 mg, 19%) was prepared as described in Example 360, except methanesulfonyl chloride (2 equiv.) was used instead of acetyl chloride. MS (ESI): mass calcd. for C₂₄H₂₉N₇O₃S, 495.2. m/z found, 496.1 [M+H]⁺. ¹H NMR (400 MHz, CDCl₃) δ 8.39 (d, J=7.2 Hz, 1H), 8.25 (s, 1H), 7.59 (d, J=0.8 Hz, 1H), 7.39 (br. s., 1H), 7.17 (s, 1H), 7.12 (s, 1H), 6.93 (dd, J=2.0, 7.2 Hz, 1H), 6.59 (s, 1H), 4.44 (s, 1H), 4.06 (s, 2H), 2.96 (s, 3H), 2.61 (s, 3H), 2.57 (s, 3H), 2.46 (s, 3H), 1.40 (s, 6H).

### Example 362: 4-[[4-[2-[(2,6-dimethylpyrimidin-4-yl)amino]pyrazolo[1,5-a]pyridin-5-yl]-6-prop-1-ynyl-3-pyridyl]amino]cyclohexanol

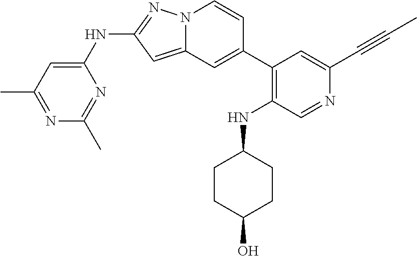 [SMILES: CC#Cc1cc(-c2ccn3nc(Nc4cc(C)nc(C)n4)cc3c2)c(N[C@H]2CC[C@@H](O)CC2)cn1]

Step A. Tert-butyl (4-(2-aminopyrazolo[1,5-a]pyridin-5-yl)-6-chloropyridin-3-yl)carbamate. A mixture consisting of tert-butyl (6-chloro-4-iodopyridin-3-yl)carbamate (9.95 g, 28.1 mmol, 1 equiv.), Intermediate 14 (12.5 g, 33.7 mmol, 1.2 equiv.), Pd(dppf)Cl₂ (1.0 g, 1.4 mmol, 0.05 equiv.), K₃PO₄ (17.9 g, 84.2 mmol, 3 equiv.), and 1,4-dioxane/H₂O (200 mL, 4:1) under N₂ atmosphere was stirred 3 h at 60° C. The reaction mixture was concentrated to dryness under reduced pressure to give the crude product, which was purified by FCC (eluent: petroleum ether:ethyl acetate)=100/0 to 0/100) to yield the title compound, tert-butyl (4-(2-aminopyrazolo[1,5-a]pyridin-5-yl)-6-chloropyridin-3-yl)carbamate, as a yellow solid (10.5 g, crude).

Step B. Tert-butyl (6-chloro-4-(2-((2,6-dimethylpyrimidin-4-yl)amino)pyrazolo[1,5-a]pyridin-5-yl)pyridin-3-yl)carbamate. A mixture consisting of tert-butyl (4-(2-aminopyrazolo[1,5-a]pyridin-5-yl)-6-chloropyridin-3-yl)carbamate (8.0 g, 22 mmol, 1 equiv.), 4-chloro-2,6-dimethylpyrimidine (3.8 g, 27 mmol, 1.2 equiv.), Pd(PPh₃)₄ (2.6 g, 2.2 mmol, 0.1 equiv.), Xantphos (2.6 g, 4.4 mmol, 0.2 equiv.), CsF (10 g, 67 mmol, 3 equiv.) and 1,4-dioxane (100 mL) was purged with N₂ for 2 min. The resultant mixture was stirred at 100° C. for 3 hr before cooling to room-temperature. The reaction mixture was concentrated to dryness under reduced pressure to give the crude product, which was purified by FCC (eluent: petroleum ether: ethyl acetate)=100/0 to 0/100) to yield the product, tert-butyl (6-chloro-4-(2-((2,6-dimethylpyrimidin-4-yl)amino)pyrazolo[1,5-a]pyridin-5-yl)pyridin-3-yl)carbamate, as a white solid (10.0 g, 68%).

Step C. Tert-butyl (4-(2-((2,6-dimethylpyrimidin-4-yl)amino)pyrazolo[1,5-a]pyridin-5-yl)-6-(prop-1-yn-1-yl)pyridin-3-yl)carbamate. A mixture consisting of tert-butyl (6-chloro-4-(2-((2,6-dimethylpyrimidin-4-yl)amino)pyrazolo[1,5-a]pyridin-5-yl)pyridin-3-yl)carbamate (3.0 g, 6.4 mmol, 1 equiv.), tributyl(prop-1-yn-1-yl)stannane (2.5 g, 7.7 mmol, 1.2 equiv.), Pd(PPh₃)₄ (744 mg, 0.644 mmol, 0.1 equiv.), LiCl (819 mg, 19.3 mmol, 3 equiv.) and DMF (30 mL) was purged with N₂ for 2 min. The resultant mixture was stirred at 90° C. overnight before cooling to room temperature. The reaction mixture was concentrated to dryness under reduced pressure to give the crude product, which was purified by FCC (eluent: petroleum ether:ethyl acetate)=100/0 to 0/100) to yield product, tert-butyl (4-(2-((2,6-dimethylpyrimidin-4-yl)amino)pyrazolo[1,5-a]pyridin-5-yl)-6-(prop-1-yn-1-yl)pyridin-3-yl)carbamate, as a yellow solid (4.0 g, 75%).

Step D. 5-(5-amino-2-(prop-1-yn-1-yl)pyridin-4-yl)-N-(2,6-dimethylpyrimidin-4-yl)pyrazolo[1,5-a]pyridin-2-amine. To a solution consisting of tert-butyl (5-(5-fluoro-2-methylpyridin-4-yl)pyrazolo[1,5-a]pyridin-2-yl)carbamate (4.0 g, 8.5 mmol, 1 equiv.) was added TFA/DCM (5 mL, 1:1). The resultant mixture was stirred at room-temperature overnight. The reaction mixture was concentrated to dryness under reduced pressure to give the crude product, then adjusted pH=7 with saturated aqueous NaHCO₃. The reaction mixture was concentrated to dryness under reduced pressure to give the crude product 5-(5-amino-2-(prop-1-yn-1-yl)pyridin-4-yl)-N-(2,6-dimethylpyrimidin-4-yl)pyrazolo[1,5-a]pyridin-2-amine (2.5 g, crude).

Step E. 4-((4-(2-((2,6-dimethylpyrimidin-4-yl)amino)pyrazolo[1,5-a]pyridin-5-yl)-6-(prop-1-yn-1-yl)pyridin-3-yl)amino)cyclohexan-1-ol. A solution consisting of 5-(5-amino-2-(prop-1-yn-1-yl)pyridin-4-yl)-N-(2,6-dimethylpyrimidin-4-yl)pyrazolo[1,5-a]pyridin-2-amine (500 mg, 1.35 mmol, 1 equiv.), 4-hydroxycyclohexanone (231 mg, 2.03 mmol, 1.5 equiv.), Ti(Oi-Pr)4 (769 mg, 2.71 mmol, 2 equiv.) in toluene (3 mL) was stirred for 0.5 hr at 120° C. via microwave. Then sodium cyanoborohydride (255 mg, 4.06 mmol, 3 equiv.) was adding to the mixture and it was stirred for 0.5 hr at 120° C. via microwave. The mixture was purified by preparative HPLC with a Phenomenex Gemini-NX 80×40 mm×3 um column (eluent: 25% to 51% (v/v) CH₃CN and H₂O with 0.05% NH₃) to afford product. the product was suspended in water (10 mL), the mixture frozen using dry ice/EtOH, and then lyophilized to dryness to afford the product, 4-((4-(2-((2,6-dimethylpyrimidin-4-yl)amino)pyrazolo[1,5-a]pyridin-5-yl)-6-(prop-1-yn-1-yl)pyridin-3-yl)amino)cyclohexan-1-ol, as a yellow solid (60 mg, 9%).

Step F. 4-[[4-[2-[(2,6-dimethylpyrimidin-4-yl)amino]pyrazolo[1,5-a]pyridin-5-yl]-6-prop-1-ynyl-3-pyridyl]amino]cyclohexanol. The material 4-((4-(2-((2,6-dimethylpyrimidin-4-yl)amino)pyrazolo[1,5-a]pyridin-5-yl)-6-(prop-1-yn-1-yl)pyridin-3-yl)amino)cyclohexan-1-ol (60 mg, 0.13 mmol) was further purified by SFC-11 over DAICEL CHIRALCEL OD 250 mm×50 mm×10 μm (eluent: 55% to 55% (v/v) 0.1% NH₃—H₂O/EtOH). The pure fractions were collected and the volatiles were removed under reduced pressure. The product was suspended in water (10 mL), the mixture frozen using dry ice/EtOH, and then lyophilized to dryness to afford the product, 4-[[4-[2-[(2,6-dimethylpyrimidin-4-yl)amino]pyrazolo[1,5-a]pyridin-5-yl]-6-prop-1-ynyl-3-pyridyl]amino]cyclohexanol (26.4 mg, 41%). MS (ESI): mass calcd. for C₂₇H₂₉N₇O, 467.2. m/z found, 468.1 [M+H]⁺. ¹H NMR (400 MHz, DMSO-d₆) δ 10.21 (s, 1H), 8.60 (d, J=7.2 Hz, 1H), 8.05 (s, 1H), 7.62 (d, J=1.2 Hz, 1H), 7.12 (s, 1H), 7.02 (s, 1H), 6.84 (s, 1H), 6.79 (dd, J=2.0, 7.2 Hz, 1H), 4.85 (d, J=7.6 Hz, 1H), 4.36 (d, J=3.6 Hz, 1H), 3.71-3.64 (m, 1H), 3.51-3.42 (m, 1H), 2.46 (s, 3H), 2.30 (s, 3H), 2.02 (s, 3H), 1.65-1.57 (m, 4H), 1.56-1.47 (m, 4H).

### Example 363: (*R)—N-(2,6-dimethylpyrimidin-4-yl)-5-[5-[(1,1-dioxothiolan-3-yl)methoxy]-2-methyl-4-pyridyl]pyrazolo[1,5-a]pyridin-2-amine

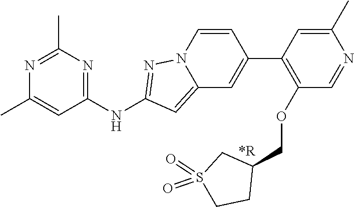

Step A. 3-(hydroxymethyl)tetrahydrothiophene 1,1-dioxide. To a mixture consisting of tetrahydrothiophene-3-carboxylic acid 1,1-dioxide (300 mg, 1.83 mmol, 1 equiv.) and THF (10 mL) was added BH₃-THF (9.1 mL, 9.1 mmol, 1 M. 5 equiv.) at 0° C. The reaction mixture was stirred at room temperature for 4 hr. The resultant mixture was added dropwise MeOH (15 mL) at 0° C., and then the mixture was stirred for 1 hr at room temperature. The reaction mixture was concentrated to dryness under reduced pressure to give the product, 3-(hydroxymethyl)tetrahydrothiophene 1,1-dioxide, as a brown oil (240 mg, crude).

Step B. (1,1-dioxidotetrahydrothiophen-3-yl)methyl 4-methylbenzenesulfonate. A mixture consisting of 3-(hydroxymethyl)tetrahydrothiophene 1,1-dioxide (240 mg, 1.60 mmol, 1 equiv.), 4-methylbenzene-1-sulfonyl chloride (396 mg, 2.08 mmol, 1.3 equiv.), N,N-dimethylpyridin-4-amine (351 mg, 2.88 mmol, 1.8 equiv.) and DCM (10 mL) was stirred at room temperature for 16 h. The reaction mixture was concentrated to dryness under reduced pressure to give the crude product, which was purified by FCC (eluent: petroleum ether: ethyl acetate=1:0 to 0:1) to afford the title compound, (1,1-dioxidotetrahydrothiophen-3-yl)methyl 4-methylbenzenesulfonate, as a white solid (290 mg, 60%).

Step C. (*R)—N-(2,6-dimethylpyrimidin-4-yl)-5-[5-[(1,1-dioxothiolan-3-yl)methoxy]-2-methyl-4-pyridyl]pyrazolo[1,5-a]pyridin-2-amine. A mixture consisting of Intermediate 16 (260 mg, 0.751 mmol, 1 equiv.), (1,1-dioxidotetrahydrothiophen-3-yl)methyl 4-methylbenzenesulfonate (274 mg, 0.901 mmol, 1.2 equiv.), Cs₂CO₃ (734 mg, 2.25 mmol, 3 equiv.) and DMF (10 mL) was stirred at 110° C. for 1 hr via microwave. The reaction mixture was filtered and purified by preparative HPLC using a Phenomenex Gemini-NX C18 75×30 mm×3 μm (eluent: 25% to 55% (v/v) water (0.05% NH₃-H₂O+10 mM NH₄HCO₃)-ACN to afford pure product. The product was suspended in water (10 mL), the mixture frozen using dry ice/ethanol, and then lyophilized to dryness to afford the material N-(2,6-dimethylpyrimidin-4-yl)-5-[5-[(1,1-dioxothiolan-3-yl)methoxy]-2-methyl-4-pyridyl]pyrazolo[1,5-a]pyridin-2-amine as a white solid (140 mg, 39%). The material was further purified by SFC over AD 250 mm×30 mm, 10 μm (eluent: 55% to 55% (v/v) supercritical CO₂ in EtOH and H₂O with 0.1% NH₃). The first eluted isomer was designated as *R isomer. The pure fractions were collected and the volatiles were removed under reduced pressure. The product was suspended in water (10 mL), the mixture frozen using dry ice/ethanol, and then lyophilized to dryness to afford the title compound (39.1 mg, 26%) as a white solid. MS (ESI): mass calcd. for C₂₄H₂₆N₆O₃S, 478.2. m/z found, 479.1 [M+H]⁺. ¹H NMR (400 MHz, DMSO-d₆) δ 10.22 (s, 1H), 8.59 (d, J=7.2 Hz, 1H), 8.34 (s, 1H), 7.80 (s, 1H), 7.37 (s, 1H), 7.07-6.98 (m, 2H), 6.88 (br. s., 1H), 4.19 (d, J=6.0 Hz, 2H), 3.27-3.16 (m, 2H), 3.12-3.03 (m, 1H), 3.00-2.93 (m, 1H), 2.90-2.79 (m, 1H), 2.47 (s, 3H), 2.46 (s, 3H), 2.31 (s, 3H), 2.28-2.21 (m, 1H), 1.98-1.87 (m, 1H).

### Example 364: (*S)—N-(2,6-dimethylpyrimidin-4-yl)-5-[5-[(1,1-dioxothiolan-3-yl)methoxy]-2-methyl-4-pyridyl]pyrazolo[1,5-a]pyridin-2-amine

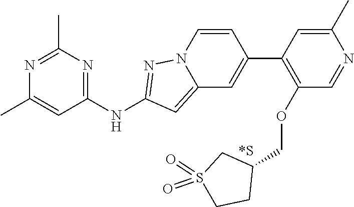

Step A. 3-(hydroxymethyl)tetrahydrothiophene 1,1-dioxide. To a mixture consisting of tetrahydrothiophene-3-carboxylic acid 1,1-dioxide (300 mg, 1.83 mmol, 1 equiv.) and THF (10 mL) was added BH3-THF (9.1 mL, 9.1 mmol, 1 M. 5 equiv.) at 0° C. The reaction mixture was stirred at room temperature for 4 hr. The resultant mixture was added dropwise MeOH (15 mL) at 0° C., and then the mixture was stirred for 1 hr at room temperature. The reaction mixture was concentrated to dryness under reduced pressure to give the product, 3-(hydroxymethyl)tetrahydrothiophene 1,1-dioxide, as a brown oil (240 mg, crude).

Step B. (1,1-dioxidotetrahydrothiophen-3-yl)methyl 4-methylbenzenesulfonate. A mixture consisting of 3-(hydroxymethyl)tetrahydrothiophene 1,1-dioxide (240 mg, 1.60 mmol, 1 equiv.), 4-methylbenzene-1-sulfonyl chloride (396 mg, 2.08 mmol, 1.3 equiv.), N,N-dimethylpyridin-4-amine (351 mg, 2.88 mmol, 1.8 equiv.) and DCM (10 mL) was stirred at room temperature for 16 h. The reaction mixture was concentrated to dryness under reduced pressure to give the crude product, which was purified by FCC (eluent: petroleum ether: ethyl acetate=1:0 to 0:1) to afford the title compound, (1,1-dioxidotetrahydrothiophen-3-yl)methyl 4-methylbenzenesulfonate, as a white solid (290 mg, 60%).

Step C. (*S)—N-(2,6-dimethylpyrimidin-4-yl)-5-[5-[(1,1-dioxothiolan-3-yl)methoxy]-2-methyl-4-pyridyl]pyrazolo[1,5-a]pyridin-2-amine. A mixture consisting of Intermediate 16 (260 mg, 0.751 mmol, 1 equiv.), (1,1-dioxidotetrahydrothiophen-3-yl)methyl 4-methylbenzenesulfonate (274 mg, 0.901 mmol, 1.2 equiv.), Cs₂CO₃ (734 mg, 2.25 mmol, 3 equiv.) and DMF (10 mL) was stirred at 110° C. for 1 hr via microwave. The reaction mixture was filtered and purified by preparative HPLC using a Phenomenex Gemini-NX C18 75×30 mm×3 μm (eluent: 25% to 55% (v/v) water (0.05% NH₃-H₂O+10 mM NH₄HCO₃)-ACN to afford pure product. The product was suspended in water (10 mL), the mixture frozen using dry ice/ethanol, and then lyophilized to dryness to afford the material N-(2,6-dimethylpyrimidin-4-yl)-5-[5-[(1,1-dioxothiolan-3-yl)methoxy]-2-methyl-4-pyridyl]pyrazolo[1,5-a]pyridin-2-amine, as a white solid (140 mg, 39%). The material was further purified by SFC over AD 250 mm×30 mm, 10 μm (eluent: 55% to 55% (v/v) supercritical CO₂ in EtOH and H₂O with 0.1% NH₃). The second eluted isomer was designated as *S isomer. The pure fractions were collected and the volatiles were removed under reduced pressure. The product was suspended in water (10 mL), the mixture frozen using dry ice/ethanol, and then lyophilized to dryness to afford the title compound (37.6 mg, 25%) as a white solid. MS (ESI): mass calcd. for C₂₄H₂₆N₆O₃S, 478.2. m/z found, 479.1 [M+H]⁺. ¹H NMR (400 MHz, DMSO-d₆) δ 10.22 (s, 1H), 8.59 (d, J=6.8 Hz, 1H), 8.34 (s, 1H), 7.80 (s, 1H), 7.37 (s, 1H), 7.07-6.97 (m, 2H), 6.87 (br. s., 1H), 4.19 (d, J=6.0 Hz, 2H), 3.27-3.16 (m, 2H), 3.11-3.04 (m, 1H), 2.99-2.93 (m, 1H), 2.88-2.81 (m, 1H), 2.47 (s, 3H), 2.46 (s, 3H), 2.30 (s, 3H), 2.28-2.21 (m, 1H), 1.98-1.86 (m, 1H).

### Example 365: (4S)-4-[[4-[2-[(2,6-dimethylpyrimidin-4-yl)amino]pyrazolo[1,5-a]pyridin-5-yl]-6-methyl-3-pyridyl]oxymethyl]-1-methyl-imidazolidin-2-one

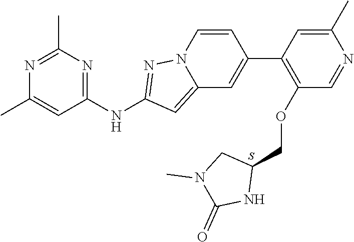

Step A. (S)-4-(hydroxymethyl)-1-methylimidazolidin-2-one. To a mixture of (S)-benzyl 1-methyl-2-oxoimidazolidine-4-carboxylate (340 mg, 1.45 mmol, 1 equiv.), CaCl₂) (96.7 mg, 0.871 mmol, 0.6 equiv.) and EtOH (10 mL) at 0° C. was added NaBH₄ (66 mg, 1.7 mmol, 1.2 equiv.). The mixture was stirred at room temperature for 12 hr. The ethanol was removed under vacuum and the residue was dissolved in sat. aq. NH₄Cl (5 mL). The mixture was extracted with EtOAc (20 mL×3) and the product was detected in the aqueous phase. The aqueous phase was frozen using dry ice/ethanol, and then lyophilized to dryness to get the crude product. To the crude product was added MeOH (5 mL) and MeCN (100 mL) to remove salt. The mixture was filtered then concentrated to give the compound (S)-4-(hydroxymethyl)-1-methylimidazolidin-2-one as a colorless oil (160 mg, 85%).

Step B. (S)-(1-methyl-2-oxoimidazolidin-4-yl)methyl 4-methylbenzenesulfonate. A mixture consisting of (S)-4-(hydroxymethyl)-1-methylimidazolidin-2-one (150 mg, 1.15 mmol, 1 equiv.), 4-methylbenzene-1-sulfonyl chloride (264 mg, 1.38 mmol, 1.2 equiv.), N,N-dimethylpyridin-4-amine (211 mg, 1.73 mmol, 1.5 equiv.) and DCM (10 mL) was stirred at room temperature for 16 h. The reaction mixture was concentrated to dryness under reduced pressure to give the crude product, which was purified by FCC (eluent: petroleum ether: ethyl acetate=1:0 to 0:1) to afford the title compound (S)-(1-methyl-2-oxoimidazolidin-4-yl)methyl 4-methylbenzenesulfonate as a white solid (120 mg, 36%).

Step C. (4S)-4-[[4-[2-[(2,6-dimethylpyrimidin-4-yl)amino]pyrazolo[1,5-a]pyridin-5-yl]-6-methyl-3-pyridyl]oxymethyl]-1-methyl-imidazolidin-2-one. A mixture consisting of Intermediate 16 (120 mg, 0.346 mmol, 1 equiv.), (S)-(1-methyl-2-oxoimidazolidin-4-yl)methyl 4-methylbenzenesulfonate (98.5 mg, 0.346 mmol, 1.2 equiv.), Cs₂CO₃ (339 mg, 1.04 mmol) and DMF (3 mL) was stirred at 110° C. for 1 h via microwave. The reaction mixture was filtered and purified by preparative HPLC using a Phenomenex Gemini-NX C18 150×30 mm×5 μm (eluent: 25% to 55% (v/v) water (0.05% NH₃—H₂O)-ACN to afford pure product. The product was suspended in water (10 mL), the mixture frozen using dry ice/ethanol, and then lyophilized to dryness to afford the title compound as a white solid. The compound was further purified by SFC over AD CHIRALPAK AS 250 mm×30 mm, 10 μm (eluent: 40% to 40% (v/v) supercritical 0.1% NH₃—H₂O EtOH). The pure fractions were collected and the volatiles were removed under reduced pressure. The product was suspended in water (10 mL), the mixture frozen using dry ice/ethanol, and then lyophilized to dryness to afford the title compound, (4S)-4-[[4-[2-[(2,6-dimethylpyrimidin-4-yl)amino]pyrazolo[1,5-a]pyridin-5-yl]-6-methyl-3-pyridyl]oxymethyl]-1-methyl-imidazolidin-2-one, as a white solid (19.7 mg, 12%).MS (ESI): mass calcd. for C₂₄H₂₆N₈O₂, 458.2. m/z found, 459.2 [M+H]⁺. ¹H NMR (400 MHz, DMSO-d₆) δ 10.21 (s, 1H), 8.53 (d, J=7.6 Hz, 1H), 8.35 (s, 1H), 7.86 (d, J=1.2 Hz, 1H), 7.39 (s, 1H), 7.04 (dd, J=2.0, 7.2 Hz, 1H), 7.01 (br. s., 1H), 6.92 (br. s., 1H), 6.64 (s, 1H), 4.12-4.03 (m, 2H), 3.95-3.87 (m, 1H), 3.49-3.43 (m, 1H), 3.21-3.15 (m, 1H), 2.56 (s, 3H), 2.48-2.45 (m, 6H), 2.30 (s, 3H).

### Example 366: 5-[2-methyl-5-[[(1S,5R, 7s)-3-oxa-9-azabicyclo[3.3.1]nonan-7-yl]oxy]-4-pyridyl]-N-[6-(trifluoromethyl)-2-pyridyl]pyrazolo[1,5-a]pyridin-2-amine

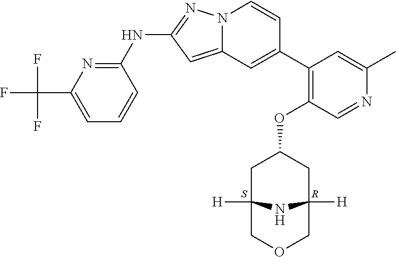

The title compound (21.9 mg, 10%) was prepared as described in Example 116, Step A-B except 2-bromo-6-(trifluoromethyl)pyridine (117 mg, 0.52 mmol) was used instead of 4-bromo-1-(difluoromethyl)-1H-pyrazole in Step A, and HCl in 1,4-dioxane was used instead of TFA in Step B. MS (ESI): mass calcd. for C₂₆H₂₅F₃N₆O₂, 510.2. m/z found, 511.3 [M+H]⁺. ¹H NMR (400 MHz, DMSO-d₆) δ 10.37 (s, 1H), 8.55 (d, J=7.2 Hz, 1H), 8.28 (s, 1H), 8.13 (d, J=1.2 Hz, 1H), 7.92-7.83 (m, 1H), 7.53 (d, J=8.4 Hz, 1H), 7.42 (s, 1H), 7.26 (d, J=7.2 Hz, 1H), 7.16 (dd, J=2.0, 7.2 Hz, 1H), 6.91 (s, 1H), 4.75-4.61 (m, 1H), 3.65-3.50 (m, 4H), 2.95 (d, J=7.2 Hz, 2H), 2.46 (s, 3H), 2.38-2.23 (m, 2H), 1.81-1.64 (m, 2H). ¹⁹F NMR (376 MHz, DMSO-d₆) δ −67.06 (s, 3F).

### Example 367: N-methyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-2-carboxamide

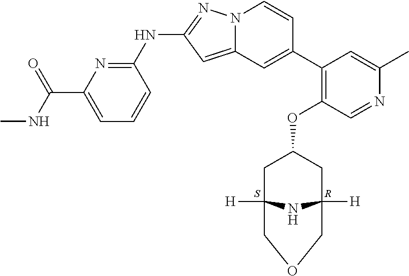

The title compound (29.5 mg, 10%) was prepared as described in Example 116, Step A-B except 6-bromo-N-methylpicolinamide (152 mg, 0.71 mmol) was used instead of 4-bromo-1-(difluoromethyl)-1H-pyrazole in Step A. MS (ESI): mass calcd. for C₂₇H₂₉N₇O₃, 499.2. m/z found, 500.2 [M+H]⁺. ¹H NMR (400 MHz, DMSO-d₆) δ 10.00 (s, 1H), 8.55 (d, J=7.2 Hz, 1H), 8.30 (s, 1H), 8.15 (q, J=4.4 Hz, 1H), 8.05 (s, 1H), 7.84-7.75 (m, 1H), 7.48 (d, J=8.4 Hz, 1H), 7.44 (d, J=7.2 Hz, 1H), 7.39 (s, 1H), 7.13 (dd, J=1.6, 6.8 Hz, 1H), 6.84 (s, 1H), 4.74-4.61 (m, 1H), 3.60-3.51 (m, 4H), 2.97-2.88 (m, 5H), 2.47 (s, 3H), 2.36-2.24 (m, 2H), 1.75-1.61 (m, 2H).

### Example 368: 5-[2-methyl-5-[[(1S,5R,7s)-3-oxa-9-azabicyclo[3.3.1]nonan-7-yl]oxy]-4-pyridyl]-N-pyridazin-4-yl-pyrazolo[1,5-a]pyridin-2-amine

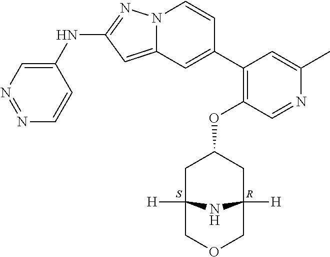

The title compound (20 mg, 9%) was prepared as described in Example 116, Step A-B except 4-bromopyridazine (113 mg, 0.71 mmol) was used instead of 4-bromo-1-(difluoromethyl)-1H-pyrazole in Step A. MS (ESI): mass calcd. for C₂₄H₂₅N₇O₂, 443.2. m/z found, 444.2 [M+H]⁺. ¹H NMR (400 MHz, DMSO-d₆) δ 10.05 (s, 1H), 9.19 (d, J=2.4 Hz, 1H), 8.84 (d, J=6.0 Hz, 1H), 8.63 (d, J=7.6 Hz, 1H), 8.30 (s, 1H), 8.02 (s, 1H), 7.87 (dd, J=3.2, 6.0 Hz, 1H), 7.38 (s, 1H), 7.16 (dd, J=2.0, 7.6 Hz, 1H), 6.27 (s, 1H), 4.76-4.59 (m, 1H), 3.63-3.44 (m, 4H), 2.98-2.87 (m, 2H), 2.46 (s, 3H), 2.36-2.22 (m, 2H), 1.75-1.56 (m, 2H).

### Example 369: N-(2-hydroxyethyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide

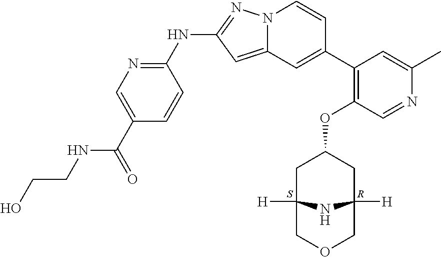

Step A: tert-butyl (1R,5S,7s)-7-((4-(2-((5-((2-hydroxyethyl)carbamoyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate. A solution consisting of tert-butyl (1R,5S,7s)-7-((4-(2-((5-(methoxycarbonyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate (Example 370, Step A, 100 mg, 0.166 mmol), 2-aminoethanol (133 mg, 2.18 mmol), EtONa (14 mg, 0.20 mmol), and EtOH (3 mL) was stirred overnight at 65° C. The reaction mixture was directly concentrated to afford the crude product (150 mg, crude).

Step B: N-(2-hydroxyethyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide. The title compound (11 mg, 14%) was prepared as described in Example 116, Step B except HCl in 1,4-dioxane was used instead of TFA and tert-butyl (1R,5S,7s)-7-((4-(2-((5-((2-hydroxyethyl)carbamoyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate was used instead of tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate. MS (ESI): mass calcd. for C₂₈H₃₁N₇O₄, 529.2. m/z found, 530.3 [M+H]⁺. ¹H NMR (400 MHz, DMSO-d₆) δ 10.20 (s, 1H), 8.74 (d, J=2.0 Hz, 1H), 8.55 (d, J=7.2 Hz, 1H), 8.37 (t, J=5.2 Hz, 1H), 8.29 (s, 1H), 8.06 (dd, J=2.0, 8.8 Hz, 1H), 8.04-7.99 (m, 1H), 7.38 (s, 1H), 7.35 (d, J=8.8 Hz, 1H), 7.13 (dd, J=2.0, 7.2 Hz, 1H), 6.85 (s, 1H), 4.76 (t, J=5.2 Hz, 1H), 4.73-4.64 (m, 1H), 3.66-3.56 (m, 4H), 3.53-3.49 (m, 2H), 3.32-3.31 (m, 2H), 3.10-3.03 (m, 2H), 2.46 (s, 3H), 2.38-2.28 (m, 2H), 1.82-1.69 (m, 2H).

### Example 370: 6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxylic Acid

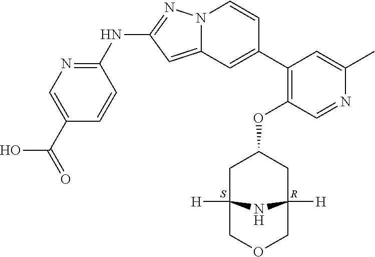

Step A. tert-butyl (1R,5S,7s)-7-((4-(2-((5-(methoxycarbonyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate. The title compound (750 mg, 96%) was prepared as described in Example 116, Step A except methyl 6-bromonicotinate (464 mg, 2.15 mmol) was used instead of 4-bromo-1-(difluoromethyl)-1H-pyrazole.

Step B: 6-((5-(5-(((1R,5S,7s)-9-(tert-butoxycarbonyl)-3-oxa-9-azabicyclo[3.3.1]nonan-7-yl)oxy)-2-methylpyridin-4-yl)pyrazolo[1,5-a]pyridin-2-yl)amino)nicotinic acid. LiOH·H₂O (42 mg, 1.0 mmol) was added to a solution consisting of tert-butyl (1R,5S,7s)-7-((4-(2-((5-(methoxycarbonyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate (120 mg, 0.200 mmol), MeOH (2 mL), and THF (1 mL). The reaction mixture was stirred overnight at 50° C. The reaction mixture was directly concentrated to afford the crude compound (150 mg, crude).

Step C. 6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxylic acid. The title compound (10 mg, 11%) was prepared as described in Example 116, Step B except HCl in 1,4-dioxane was used instead of TFA and 6-((5-(5-(((1R,5S,7s)-9-(tert-butoxycarbonyl)-3-oxa-9-azabicyclo[3.3.1]nonan-7-yl)oxy)-2-methylpyridin-4-yl)pyrazolo[1,5-a]pyridin-2-yl)amino)nicotinic acid was used instead of tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate. MS (ESI): mass calcd. for C₂₆H₂₆N₆O₄, 486.2. m/z found, 487.2 [M+H]⁺. ¹H NMR (400 MHz, DMSO-d₆) δ 10.48 (s, 1H), 9.92-9.65 (m, 2H), 8.78 (d, J=2.0 Hz, 1H), 8.62 (d, J=7.2 Hz, 1H), 8.45 (s, 1H), 8.21 (s, 1H), 8.09 (dd, J=2.4, 8.8 Hz, 1H), 7.83 (s, 1H), 7.38 (d, J=8.8 Hz, 1H), 7.24 (dd, J=1.6, 7.2 Hz, 1H), 6.95 (s, 1H), 4.98-4.88 (m, 1H), 4.00-3.74 (m, 4H), 3.67-3.51 (m, 2H), 2.69-2.62 (m, 5H), 2.20-2.11 (m, 2H)

### Example 371: N-(2-hydroxy-2-methyl-propyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide

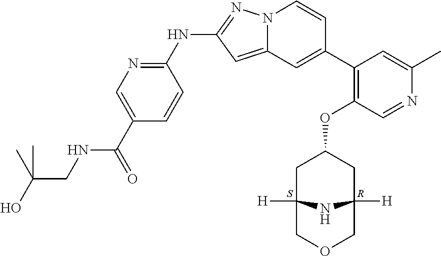

Step A: tert-butyl (1R,5S,7s)-7-((4-(2-((5-((2-hydroxy-2-methylpropyl)carbamoyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate. To a solution of 6-((5-(5-(((1R,5S,7s)-9-(tert-butoxycarbonyl)-3-oxa-9-azabicyclo[3.3.1]nonan-7-yl)oxy)-2-methylpyridin-4-yl)pyrazolo[1,5-a]pyridin-2-yl)amino)nicotinic acid (Example 370, Step B, 100 mg, 0.170 mmol) and 1-amino-2-methylpropan-2-ol (7.6 mg, 0.085 mmol) in DMF (3 mL) was added DIEA (33.0 mg, 0.256 mmol) and HATU (64.8 mg, 0.170 mmol). The mixture was stirred at room temp for 16 h. The reaction mixture was filtered and the filtrate was purified by preparative HPLC using a YMC-Triart Prep C18 250×50 mm×10 um column (eluent: 36% to 66% (v/v) CH₃CN and H₂O with 0.04% NH₃). The product was suspended in water (10 mL), the mixture frozen using dry ice/ethanol, and then lyophilized to dryness to afford the title compound (46 mg, 82%) as a white solid.

Step B: N-(2-hydroxy-2-methyl-propyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide. The title compound (34 mg, 88%) was prepared as described in Example 116, Step B except tert-butyl (1R,5S,7s)-7-((4-(2-((5-((2-hydroxy-2-methylpropyl)carbamoyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate was used instead of tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate. MS (ESI): mass calcd. for C₃₀H₃₅N₇O₄, 557.3. m/z found, 558.3 [M+H]+. ¹H NMR (400 MHz, CDCl₃) δ 8.75 (d, J=2.0 Hz, 1H), 8.34 (d, J=7.6 Hz, 1H), 8.23 (s, 1H), 8.07 (dd, J=2.4, 8.8 Hz, 1H), 7.91 (d, J=1.2 Hz, 1H), 7.74 (s, 1H), 7.48 (d, J=8.8 Hz, 1H), 7.21 (s, 1H), 7.12 (dd, J=2.0, 7.2 Hz, 1H), 6.59-6.51 (m, 2H), 4.63-4.58 (m, 1H), 3.78-3.71 (m, 4H), 3.50 (d, J=6.0 Hz, 2H), 3.05 (d, J=7.6 Hz, 2H), 2.56 (s, 3H), 2.39-2.32 (m, 2H), 2.02-1.95 (m, 2H), 1.32 (s, 6H).

### Example 372: 5-[2-methyl-5-[[(1S,5R,7s)-3-oxa-9-azabicyclo[3.3.1]nonan-7-yl]oxy]-4-pyridyl]-N-(2-methylpyrimidin-4-yl)pyrazolo[1,5-a]pyridin-2-amine

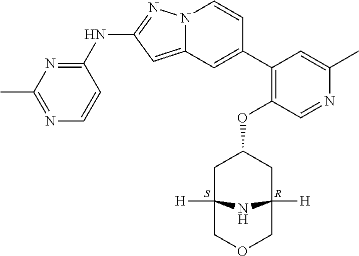

The title compound (51 mg, 22%) was prepared as described in Example 116, Step A-B except 4-chloro-2-methylpyrimidine (99 mg, 0.77 mmol) was used instead of 4-bromo-1-(difluoromethyl)-1H-pyrazole in Step A. MS (ESI): mass calcd. for C₂₅H₂₇N₇O₂, 457.2. m/z found, 458.3 [M+H]⁺. ¹H NMR (400 MHz, CD₃OD) δ 8.41 (d, J=7.2 Hz, 1H), 8.21 (d, J=6.0 Hz, 1H), 8.17 (s, 1H), 8.11 (d, J=0.8 Hz, 1H), 7.41 (s, 1H), 7.22-7.15 (m, 2H), 6.87 (s, 1H), 4.73-4.66 (m, 1H), 3.84-3.68 (m, 4H), 2.98 (d, J=6.8 Hz, 2H), 2.56 (s, 3H), 2.53 (s, 3H), 2.43-2.33 (m, 2H), 2.01-1.93 (m, 2H).

### Example 373: 6-[[5-[2-methyl-5-[[(1R,5S,7s)-3-oxa-9-azabicyclo[3.3.1]nonan-7-yl]oxy]-4-pyridyl]pyrazolo[1,5-a]pyridin-2-yl]amino]-N-(tetrahydropyran-4-ylmethyl)pyridine-3-carboxamide

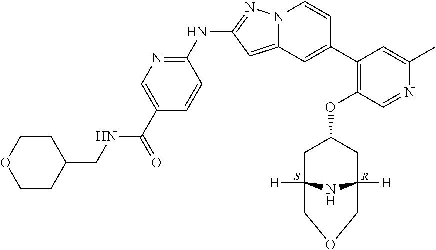

Step A: tert-butyl (1R,5S,7s)-7-((6-methyl-4-(2-((5-(((tetrahydro-2H-pyran-4-yl)methyl)carbamoyl)pyridin-2-yl)amino)pyrazolo[1,5-a]pyridin-5-yl)pyridin-3-yl)oxy)-3-oxa-9-azabicyclo[3.3.1]nonane-9-carboxylate. A mixture consisting of tert-butyl (1R,5S,7s)-7-((4-(2-((5-(methoxycarbonyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate (Example 370, Step A, 70 mg, 0.12 mmol), (tetrahydro-2H-pyran-4-yl)methanamine (27 mg, 0.23 mmol), DIEA (60 mg, 0.46 mmol), HATU (32 mg, 0.23 mmol) and DMF (3 mL) was stirred at 25° C. for 4 h before cooling to room temperature. The reaction mixture was poured onto 40 mL of water. During this period, white precipitate was formed. It was collected by filtration and air-dried. The precipitate was concentrated under reduced pressure to give the title product (70 mg, 87%).

Step B: 6-[[5-[2-methyl-5-[[(1R,5S,7s)-3-oxa-9-azabicyclo[3.3.1]nonan-7-yl]oxy]-4-pyridyl]pyrazolo[1,5-a]pyridin-2-yl]amino]-N-(tetrahydropyran-4-ylmethyl)pyridine-3-carboxamide. The title compound (5.7 mg, 9%) was prepared as described in Example 116, Step B except tert-butyl (1R,5S,7s)-7-((6-methyl-4-(2-((5-(((tetrahydro-2H-pyran-4-yl)methyl)carbamoyl)pyridin-2-yl)amino)pyrazolo[1,5-a]pyridin-5-yl)pyridin-3-yl)oxy)-3-oxa-9-azabicyclo[3.3.1]nonane-9-carboxylate (70 mg, 0.10 mmol) was used instead of tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate. MS (ESI): mass calcd. for C₃₂H₃₇N₇O₄, 583.3. m/z found, 584.2 [M+H]⁺. ¹H NMR (400 MHz, CD₃OD) δ 8.71 (d, J=2.0 Hz, 1H), 8.39 (d, J=7.2 Hz, 1H), 8.16 (s, 1H), 8.07-8.02 (m, 2H), 7.41 (s, 1H), 7.38 (d, J=8.8 Hz, 1H), 7.14 (dd, J=2.0, 7.6 Hz, 1H), 6.80 (s, 1H), 4.75-4.67 (m, 1H), 3.96 (dd, J=2.8, 10.8 Hz, 2H), 3.88-3.75 (m, 4H), 3.46-3.37 (m, 2H), 3.28 (d, J=6.8 Hz, 2H), 3.17 (d, J=6.4 Hz, 2H), 2.53 (s, 3H), 2.48-2.39 (m, 2H), 2.12-2.05 (m, 2H), 1.97-1.84 (m, 1H), 1.74-1.66 (m, 2H), 1.40-1.27 (m, 2H).

### Example 374: 5-[2-methyl-5-[[(1S,5R,7s)-3-oxa-9-azabicyclo[3.3.1]nonan-7-yl]oxy]-4-pyridyl]-N-[5-(trifluoromethyl)-2-pyridyl]pyrazolo[1,5-a]pyridin-2-amine

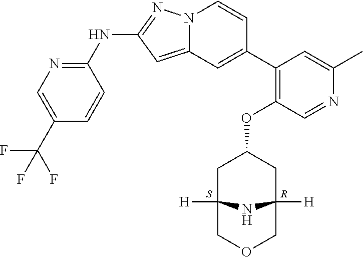

The title compound (22 mg, 18%) was prepared as described in Example 116, Step A-B except 2-bromo-5-(trifluoromethyl)pyridine (194 mg, 0.86 mmol) was used instead of 4-bromo-1-(difluoromethyl)-1H-pyrazole in Step A, and HCl in 1,4-dioxane was used instead of TFA in Step B. MS (ESI): mass calcd. for C₂₆H₂₅F₃N₆O₂, 510.2. m/z found, 511.2 [M+H]⁺. ¹H NMR (400 MHz, DMSO-d₆) δ 10.46 (s, 1H), 8.59-8.55 (m, 2H), 8.30 (s, 1H), 8.03 (d, J=0.8 Hz, 1H), 7.96 (dd, J=2.0, 8.8 Hz, 1H), 7.45 (d, J=8.8 Hz, 1H), 7.37 (s, 1H), 7.14 (dd, J=1.6, 6.8 Hz, 1H), 6.89 (s, 1H), 4.72-4.64 (m, 1H), 3.60-3.49 (m, 4H), 2.93 (d, J=8.0 Hz, 2H), 2.46 (s, 3H), 2.34-2.25 (m, 2H), 1.70-1.62 (m, 2H) 19F NMR (376 MHz, DMSO-d₆) δ −59.60 (s, 3F)

### Example 375: 5-(5-(((1R,5S,7s)-3-oxa-9-azabicyclo[3.3.1]nonan-7-yl)oxy)-2-methylpyridin-4-yl)-N-(5-(trifluoromethyl)pyrazin-2-yl)pyrazolo[1,5-a]pyridin-2-amine

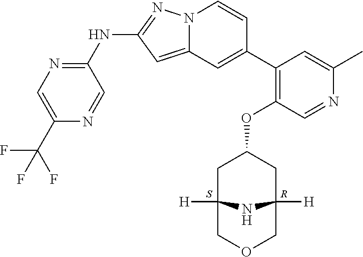

The title compound (64 mg, 38%) was prepared as described in Example 116, Step A-B except 2-chloro-5-(trifluoromethyl)pyrazine (173 mg, 0.95 mmol) was used instead of 4-bromo-1-(difluoromethyl)-1H-pyrazole in Step A. MS (ESI): mass calcd. for C₂₅H₂₄F₃N₇O₂, 511.2. m/z found, 512.3 [M+H]⁺. ¹H NMR (400 MHz, CD₃OD) δ 8.63 (s, 1H), 8.57 (s, 1H), 8.43 (d, J=7.2 Hz, 1H), 8.17 (s, 1H), 8.12-8.08 (m, 1H), 7.41 (s, 1H), 7.21 (dd, J=1.6, 7.2 Hz, 1H), 6.96 (s, 1H), 4.73-4.65 (m, 1H), 3.83-3.76 (m, 2H), 3.74-3.68 (m, 2H), 2.98 (d, J=6.8 Hz, 2H), 2.53 (s, 3H), 2.43-2.32 (m, 2H), 2.01-1.92 (m, 2H) ¹⁹F NMR (376 MHz, DMSO-d₆) δ −64.92 (s, 3F).

### Example 376: 6-[[5-[2-methyl-5-[[(1R,5S,7s)-3-oxa-9-azabicyclo[3.3.1]nonan-7-yl]oxy]-4-pyridyl]pyrazolo[1,5-a]pyridin-2-yl]amino]pyridine-3-carboxamide

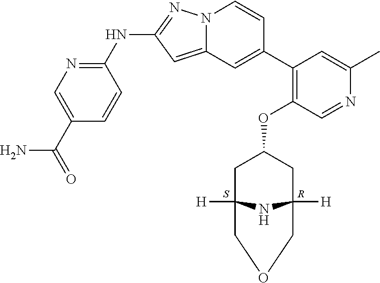

Step A: tert-butyl (1R,5S,7s)-7-((4-(2-((5-carbamoylpyridin-2-yl)amino)pyrazolo[1,5-a]pyridin-5-yl)-6-methylpyridin-3-yl)oxy)-3-oxa-9-azabicyclo[3.3.1]nonane-9-carboxylate. To a solution of 6-((5-(5-(((1R,5S,7s)-9-(tert-butoxycarbonyl)-3-oxa-9-azabicyclo[3.3.1]nonan-7-yl)oxy)-2-methylpyridin-4-yl)pyrazolo[1,5-a]pyridin-2-yl)amino)nicotinic acid (Example 370, Step B, 80 mg, 0.14 mmol), ammonia hydrochloride (15 mg, 0.27 mmol), and HATU (104 mg, 0.273 mmol) in DMF (2 mL) was added DIEA (106 mg, 0.818 mmol). The mixture was stirred at room temperature overnight. The reaction mixture was purified by preparative HPLC DG with Phenomenex Gemini-NX 150×30 mm×5 um (eluent: 15% to 37% (v/v) CH₃CN and aqueous HCl (0.006 N)). The product was suspended in water (10 mL), the mixture frozen using dry ice/ethanol, and then lyophilized to dryness to afford the title compound (102 mg) as a yellow solid.

Step B: 6-[[5-[2-methyl-5-[[(1R,5S,7s)-3-oxa-9-azabicyclo[3.3.1]nonan-7-yl]oxy]-4-pyridyl]pyrazolo[1,5-a]pyridin-2-yl]amino]pyridine-3-carboxamide. The title compound (35 mg, 53%) was prepared as described in Example 116, Step B except tert-butyl (1R,5S,7s)-7-((4-(2-((5-carbamoylpyridin-2-yl)amino)pyrazolo[1,5-a]pyridin-5-yl)-6-methylpyridin-3-yl)oxy)-3-oxa-9-azabicyclo[3.3.1]nonane-9-carboxylate (80 mg, 0.14 mmol) was used instead of tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate and HCl in 1,4-dioxane was used instead of TFA. MS (ESI): mass calcd. for C₂₆H₂₇N₇O₃, 485.2. m/z found, 486.3 [M+H]⁺. ¹H NMR (400 MHz, DMSO-d₆) δ 10.23 (s, 1H), 8.76 (d, J=1.6 Hz, 1H), 8.56 (d, J=7.6 Hz, 1H), 8.30 (s, 1H), 8.07 (dd, J=2.4, 8.8 Hz, 1H), 8.03 (d, J=1.2 Hz, 1H), 7.89 (br. s., 1H), 7.38 (s, 1H), 7.34 (d, J=8.8 Hz, 1H), 7.29 (br. s., 1H), 7.12 (dd, J=2.0, 7.2 Hz, 1H), 6.87 (s, 1H), 4.73-4.63 (m, 1H), 3.65-3.47 (m, 4H), 2.98-2.88 (m, 2H), 2.46 (s, 3H), 2.36-2.23 (m, 2H), 1.72-1.61 (m, 2H)

### Example 377: 6-[[5-[2-methyl-5-[[(1R,5S,7s)-3-oxa-9-azabicyclo[3.3.1]nonan-7-yl]oxy]-4-pyridyl]pyrazolo[1,5-a]pyridin-2-yl]amino]-N-propyl-pyridine-3-carboxamide

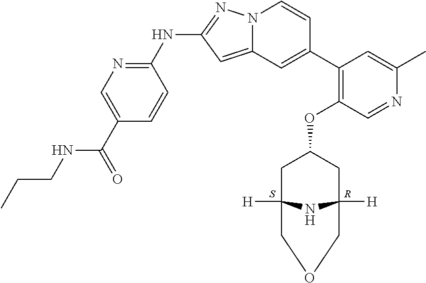

The title compound (13 mg, 23%) was prepared as described in Example 371, except propan-1-amine (25 mg, 0.43 mmol) was used instead of 1-amino-2-methylpropan-2-ol in Step A. MS (ESI): mass calcd. for C₂₉H₃₃N₇O₃, 527.3. m/z found, 528.2 [M+H]⁺. ¹H NMR (400 MHz, DMSO-d₆) δ 10.20 (s, 1H), 8.73 (d, J=2.4 Hz, 1H), 8.55 (d, J=7.2 Hz, 1H), 8.36 (t, J=5.2, 1H), 8.29 (s, 1H), 8.08-7.99 (m, 2H), 7.38 (s, 1H), 7.35 (d, J=8.8 Hz, 1H), 7.13 (dd, J=2.0, 7.2 Hz, 1H), 6.85 (s, 1H), 4.75-4.64 (m, 1H), 3.76-3.53 (m, 4H), 3.23-3.19 (m, 2H), 3.14-3.05 (m, 2H), 2.46 (s, 3H), 2.40-2.29 (m, 2H), 1.85-1.73 (m, 2H), 1.58-1.50 (m, 2H), 0.90 (t, J=7.6 Hz, 3H).

### Example 378: 5-[2-methyl-5-[[(1S,5R,7s)-3-oxa-9-azabicyclo[3.3.1]nonan-7-yl]oxy]-4-pyridyl]-N-[4-(trifluoromethyl)-2-pyridyl]pyrazolo[1,5-a]pyridin-2-amine

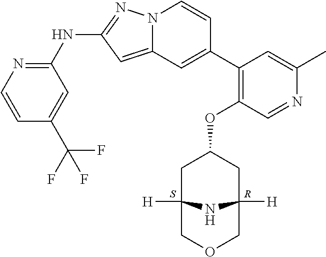

The title compound (36 mg, 23%) was prepared as described in Example 116, Step A-B except 2-bromo-4-(trifluoromethyl)pyridine (243 mg, 1.07 mmol) was used instead of 4-bromo-1-(difluoromethyl)-1H-pyrazole in Step A. MS (ESI): mass calcd. for C₂₆H₂₅F₃N₆O₂, 510.2. m/z found, 511.2 [M+H]⁺. ¹H NMR (400 MHz, DMSO-d₆) δ 10.31 (s, 1H), 8.57 (d, J=7.2 Hz, 1H), 8.48 (d, J=5.2 Hz, 1H), 8.30 (s, 1H), 8.02 (s, 1H), 7.67 (s, 1H), 7.38 (s, 1H), 7.13 (dd, J=1.6, 7.2 Hz, 1H), 7.11 (d, J=5.2 Hz, 1H), 6.85 (s, 1H), 4.77-4.59 (m, 1H), 3.64-3.59 (m, 2H), 3.57-3.52 (m, 2H), 3.01 (d, J=7.6 Hz, 2H), 2.46 (s, 3H), 2.38-2.26 (m, 2H), 1.72 (dd, J=5.2, 13.6 Hz, 2H).

### Example 379: 5-[2-methyl-5-[[(1S,5R,7s)-3-oxa-9-azabicyclo[3.3.1]nonan-7-yl]oxy]-4-pyridyl]-N-[1-methyl-5-(trifluoromethyl)pyrazol-3-yl]pyrazolo[1,5-a]pyridin-2-amine

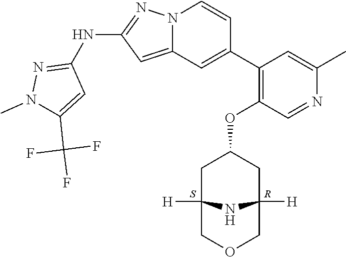

Step A: ‘Mixture of 3-bromo-1-methyl-5-(trifluoromethyl)-1H-pyrazole and 5-bromo-1-methyl-3-(trifluoromethyl)-1H-pyrazole.’ Cesium carbonate (1.8 g, 5.6 mmol) was added in portions to a 0° C. (ice/water) solution consisting of 3-bromo-5-(trifluoromethyl)-1H-pyrazole (600 mg, 2.79 mmol) and DMSO (10 mL). The resulting mixture was stirred at 0° C. for 10 min. Methyl iodide (4.31 mg, 30.37 mmol) was added dropwise into the mixture at 0° C. (ice/water). The mixture was warmed to room temperature and stirred for 5 hours. The reaction mixture was poured into water (30 mL) and extracted with ethyl acetate (30×3 mL). The combined organic extracts were dried over Na₂SO₄, filtered, and evaporated to dryness under reduced pressure to give the crude product (600 mg) as a reddish oil.

Step B: 5-[2-methyl-5-[[(1S,5R,7s)-3-oxa-9-azabicyclo[3.3.1]nonan-7-yl]oxy]-4-pyridyl]-N-[1-methyl-5-(trifluoromethyl)pyrazol-3-yl]pyrazolo[1,5-a]pyridin-2-amine. The title compound (36 mg, 22%) was prepared as described in Example 116, Step A-B except a ‘mixture of 3-bromo-1-methyl-5-(trifluoromethyl)-1H-pyrazole and 5-bromo-1-methyl-3-(trifluoromethyl)-1H-pyrazole’ (369 mg, 1.61 mmol) was used instead of 4-bromo-1-(difluoromethyl)-1H-pyrazole in Step A, and HCl in 1,4-dioxane was used instead of TFA in Step B to deprotect the correct pyrazole isomer product (see Example 403 for the other pyrazole isomer product that was also isolated alongside this). MS (ESI): mass calcd. for C₂₅H₂₆F₃N702, 513.2. m/z found, 514.2 [M+H]⁺. ¹H NMR (400 MHz, DMSO-d₆) δ 9.48 (s, 1H), 8.50 (d, J=7.2 Hz, 1H), 8.28 (s, 1H), 7.91 (d, J=1.2 Hz, 1H), 7.35 (s, 1H), 7.03 (dd, J=2.0, 7.2 Hz, 1H), 6.72 (s, 1H), 6.32 (s, 1H), 4.73-4.55 (m, 1H), 3.86 (s, 3H), 3.59-3.54 (m, 2H), 3.53-3.48 (m, 2H), 2.93 (d, J=8.0 Hz, 2H), 2.45 (s, 3H), 2.36-2.21 (m, 2H), 1.70-1.60 (m, 2H). ¹⁹F NMR (376 MHz, DMSO-d₆) δ −59.2 (s, 3F).

### Example 380: 5-[2-methyl-5-[[(1R,5S,7s)-3-oxa-9-azabicyclo[3.3.1]nonan-7-yl]oxy]-4-pyridyl]-N-(1-methyltriazol-4-yl)pyrazolo[1,5-a]pyridin-2-amine

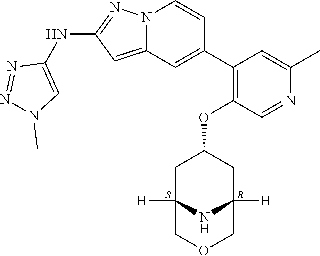

The title compound (65 mg, 34%) was prepared as described in Example 116, Step A-B except 4-bromo-1-methyl-1H-1,2,3-triazole (139 mg, 0.86 mmol) was used instead of 4-bromo-1-(difluoromethyl)-1H-pyrazole in Step A, and HCl in 1,4-dioxane was used instead of TFA in Step B. MS (ESI): mass calcd. for C₂₃H₂₆N₈O₂, 446.2. m/z found, 447.2 [M+H]⁺. ¹H NMR (400 MHz, DMSO-d₆) δ 9.49 (s, 1H), 8.49 (d, J=7.2 Hz, 1H), 8.28 (s, 1H), 7.92 (d, J=1.2 Hz, 1H), 7.64 (s, 1H), 7.36 (s, 1H), 7.04 (dd, J=2.0, 7.2 Hz, 1H), 6.25 (s, 1H), 4.74-4.60 (m, 1H), 4.04 (s, 3H), 3.69-3.62 (m, 2H), 3.61-3.56 (m, 2H), 3.10 (d, J=7.2 Hz, 2H), 2.46 (s, 3H), 2.38-2.30 (m, 2H), 1.83-1.73 (m, 2H).

### Example 381: 5-[2-methyl-5-[[(1S,5R,7s)-3-oxa-9-azabicyclo[3.3.1]nonan-7-yl]oxy]-4-pyridyl]-N-(5-methylpyrazin-2-yl)pyrazolo[1,5-a]pyridin-2-amine

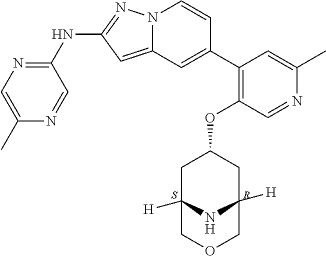

The title compound (65 mg, 51%) was prepared as described in Example 116, Step A-B except 2-bromo-5-methylpyrazine (111 mg, 0.64 mmol) was used instead of 4-bromo-1-(difluoromethyl)-1H-pyrazole in Step A. MS (ESI): mass calcd. for C₂₅H₂₇N₇O₂, 457.2. m/z found, 458.3 [M+H]⁺. ¹H NMR (400 MHz, DMSO-d₆) δ 10.07 (s, 1H), 8.56 (s, 1H), 8.55 (d, J=7.2 Hz, 1H), 8.29 (s, 1H), 8.12 (s, 1H), 7.99 (s, 1H), 7.36 (s, 1H), 7.11 (dd, J=1.6, 7.2 Hz, 1H), 6.81 (s, 1H), 4.70-4.63 (m, 1H), 3.60-3.54 (m, 2H), 3.54-3.48 (m, 2H), 2.96-2.90 (m, 2H), 2.46 (s, 3H), 2.38 (s, 3H), 2.33-2.25 (m, 2H), 1.70-1.62 (m, 2H).

### Example 382: N-(2,6-dimethylpyrimidin-4-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine

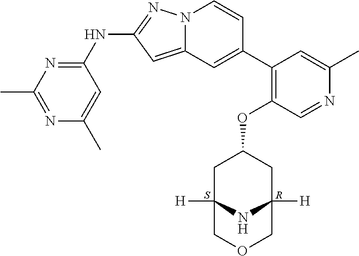

The title compound (140 mg, 59%) was prepared as described in Example 116, Step A-B except 4-chloro-2,6-dimethylpyrimidine (119 mg, 0.84 mmol) was used instead of 4-bromo-1-(difluoromethyl)-1H-pyrazole in Step A. MS (ESI): mass calcd. for C₂₆H₂₉N₇O₂, 471.2. m/z found, 472.3 [M+H]⁺. ¹H NMR (400 MHz, DMSO-d₆) δ 10.22 (s, 1H), 8.57 (d, J=7.2 Hz, 1H), 8.30 (s, 1H), 8.04 (s, 1H), 7.38 (s, 1H), 7.15 (dd, J=2.0, 7.2 Hz, 1H), 7.02 (br. s, 1H), 6.84 (br. s, 1H), 4.74-4.60 (m, 1H), 3.61-3.55 (m, 2H), 3.54-3.49 (m, 2H), 2.93 (d, J=8.0 Hz, 2H), 2.48-2.44 (m, 6H), 2.36-2.22 (m, 5H), 1.71-1.61 (m, 2H).

### Example 383: N,N-dimethyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide

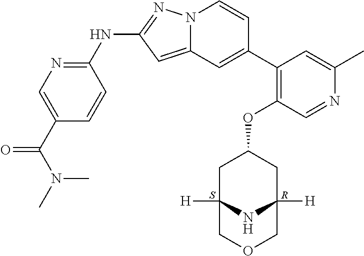

The title compound (45 mg, 24%) was prepared as described in Example 116, Step A-B except 6-bromo-N,N-dimethylnicotinamide (108 mg, 0.47 mmol) was used instead of 4-bromo-1-(difluoromethyl)-1H-pyrazole in Step A. MS (ESI): mass calcd. for C₂₈H₃₁N₇O₃, 513.2. m/z found, 514.2 [M+H]⁺. ¹H NMR (400 MHz, DMSO-d₆) δ 10.13 (s, 1H), 8.54 (d, J=7.2 Hz, 1H), 8.33 (d, J=2.0 Hz, 1H), 8.30 (s, 1H), 7.99 (s, 1H), 7.73 (dd, J=2.4, 8.8 Hz, 1H), 7.36 (s, 1H), 7.34 (d, J=8.8 Hz, 1H), 7.11 (dd, J=2.0, 7.2 Hz, 1H), 6.86 (s, 1H), 4.74-4.56 (m, 1H), 3.59-3.54 (m, 2H), 3.53-3.48 (m, 2H), 3.01 (s, 6H), 2.96-2.89 (m, 2H), 2.46 (s, 3H), 2.34-2.25 (m, 2H), 1.70-1.61 (m, 2H).

### Example 384: N-ethyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide

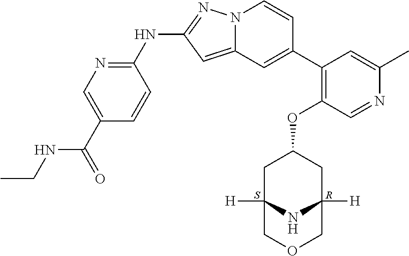

Step A: tert-butyl (1R,5S,7s)-7-((4-(2-((5-(ethylcarbamoyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate. A mixture consisting of tert-butyl (1R,5S,7s)-7-((4-(2-((5-(methoxycarbonyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate (Example 370, Step A, 100 mg, 0.166 mmol), EtNH2 (751 mg, 16.6 mmol), NaOMe (9.89 mg, 0.183 mmol) and MeOH (3 mL) was stirred at 65° C. overnight before cooling to room temperature. The reaction mixture was concentrated to dryness under reduced pressure to give the crude product, which was used without further purification in Step B.

Step B: N-ethyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide. The title compound (44 mg, 41%) was prepared as described in Example 116, Step B except tert-butyl (1R,5S,7s)-7-((4-(2-((5-(ethylcarbamoyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate (125 mg, 0.20 mmol) was used instead of tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate. MS (ESI): mass calcd. for C₂₈H₃₁N₇O₃, 513.2. m/z found, 514.3 [M+H]⁺. ¹H NMR (400 MHz, DMSO-d₆) δ 10.20 (s, 1H), 8.73 (d, J=1.6 Hz, 1H), 8.55 (d, J=7.2 Hz, 1H), 8.37 (t, J=5.2 Hz, 1H), 8.30 (s, 1H), 8.12-8.00 (m, 2H), 7.37 (s, 1H), 7.35 (d, J=8.8 Hz, 1H), 7.22-7.05 (m, 1H), 6.86 (s, 1H), 4.75-4.61 (m, 1H), 3.61-3.55 (m, 2H), 3.54-3.49 (m, 2H), 3.31-3.28 (m, 2H), 2.97-2.89 (m, 2H), 2.46 (s, 3H), 2.37-2.19 (m, 2H), 1.74-1.58 (m, 2H), 1.13 (t, J=7.2 Hz, 3H).

### Example 385: N-ethyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridazine-3-carboxamide

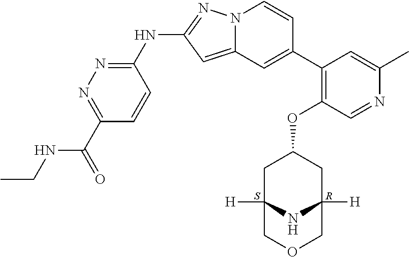

Step A: tert-butyl (1R,5S,7s)-7-((4-(2-((6-(ethoxycarbonyl)pyridazin-3-yl)amino)pyrazolo[1,5-a]pyridin-5-yl)-6-methylpyridin-3-yl)oxy)-3-oxa-9-azabicyclo[3.3.1]nonane-9-carboxylate. A mixture consisting of tert-butyl (1R,5S,7s)-7-((4-(2-Aminopyrazolo[1,5-a]pyridin-5-yl)-6-methylpyridin-3-yl)oxy)-3-oxa-9-azabicyclo[3.3.1]nonane-9-carboxylate (Intermediate 9, 100 mg, 0.215 mmol), ethyl 6-chloropyridazine-3-carboxylate (80.2 mg, 0.430 mmol), Pd(OAc)2 (8.7 mg, 0.039 mmol), Xantphos (50 mg, 0.086 mmol), CsF (97.9 mg, 0.644 mmol) and toluene (5 mL) was purged with argon for 2 min. The resulting mixture was stirred at 100° C. for 10 h before cooling to room temperature. The reaction mixture was concentrated to dryness under reduced pressure to give the crude product, which was purified by FCC (eluent: petroleum ether: (ethyl acetate:ethanol=3:1)=100/0 to 50/50) to yield the title compound (180 mg, 94%) as a light yellow solid.

Step B: tert-butyl (1R,5S,7s)-7-((4-(2-((6-(ethylcarbamoyl)pyridazin-3-yl)amino)pyrazolo[1,5-a]pyridin-5-yl)-6-methylpyridin-3-yl)oxy)-3-oxa-9-azabicyclo[3.3.1]nonane-9-carboxylate. The title compound (150 mg, 70%) was prepared as described in Example 384, Step A except tert-butyl (1R,5S,7s)-7-((4-(2-((6-(ethoxycarbonyl)pyridazin-3-yl)amino)pyrazolo[1,5-a]pyridin-5-yl)-6-methylpyridin-3-yl)oxy)-3-oxa-9-azabicyclo[3.3.1]nonane-9-carboxylate (150 mg, 0.24 mmol) was used instead of tert-butyl (1R,5S,7s)-7-((4-(2-((5-(methoxycarbonyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate.

Step C: N-ethyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridazine-3-carboxamide. The title compound (58 mg, 54%) was prepared as described in Example 116, Step B except tert-butyl (1R,5S,7s)-7-((4-(2-((6-(ethylcarbamoyl)pyridazin-3-yl)amino)pyrazolo[1,5-a]pyridin-5-yl)-6-methylpyridin-3-yl)oxy)-3-oxa-9-azabicyclo[3.3.1]nonane-9-carboxylate (125 mg, 0.20 mmol) was used instead of tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate. MS (ESI): mass calcd. for C₂₇H₃₀N₈O₃, 514.2. m/z found, 515.2 [M+H]⁺. ¹H NMR (400 MHz, DMSO-d₆) δ 10.64 (s, 1H), 9.03 (t, J=5.2 Hz, 1H), 8.59 (d, J=7.2 Hz, 1H), 8.30 (s, 1H), 8.11 (s, 1H), 8.02 (d, J=9.2 Hz, 1H), 7.71 (d, J=9.2 Hz, 1H), 7.39 (s, 1H), 7.20-7.12 (m, 1H), 6.96 (s, 1H), 4.77-4.61 (m, 1H), 3.64-3.49 (m, 4H), 3.39-3.36 (m, 2H), 2.96-2.89 (m, 2H), 2.47 (s, 3H), 2.38-2.22 (m, 2H), 1.74-1.55 (m, 2H), 1.15 (t, J=7.2 Hz, 3H).

### Example 386: N-ethyl-5-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyrazine-2-carboxamide

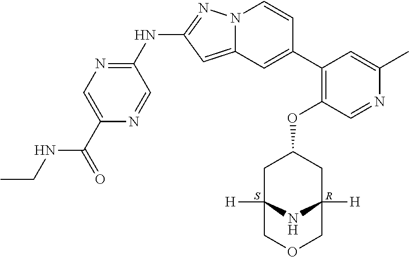

Step A: tert-butyl (1R,5S,7s)-7-((4-(2-((5-(ethoxycarbonyl)pyrazin-2-yl)amino)pyrazolo[1,5-a]pyridin-5-yl)-6-methylpyridin-3-yl)oxy)-3-oxa-9-azabicyclo[3.3.1]nonane-9-carboxylate. The title compound (190 mg, 39%) was prepared as described in Example 385, Step A except ethyl 5-chloropyrazine-2-carboxylate (120 mg, 0.64 mmol) was used instead of ethyl 6-chloropyridazine-3-carboxylate.

Step B: tert-butyl (1R,5S,7s)-7-((4-(2-((5-(ethylcarbamoyl)pyrazin-2-yl)amino)pyrazolo[1,5-a]pyridin-5-yl)-6-methylpyridin-3-yl)oxy)-3-oxa-9-azabicyclo[3.3.1]nonane-9-carboxylate. The title compound (190 mg, 58%) was prepared as described in Example 384, Step A except tert-butyl (1R,5S,7s)-7-((4-(2-((5-(ethoxycarbonyl)pyrazin-2-yl)amino)pyrazolo[1,5-a]pyridin-5-yl)-6-methylpyridin-3-yl)oxy)-3-oxa-9-azabicyclo[3.3.1]nonane-9-carboxylate (150 mg, 0.24 mmol) was used instead of tert-butyl (1R,5S,7s)-7-((4-(2-((5-(methoxycarbonyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate.

Step C: N-ethyl-5-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyrazine-2-carboxamide. The title compound (21.4 mg, 15%) was prepared as described in Example 116, Step B except tert-butyl (1R,5S,7s)-7-((4-(2-((5-(ethylcarbamoyl)pyrazin-2-yl)amino)pyrazolo[1,5-a]pyridin-5-yl)-6-methylpyridin-3-yl)oxy)-3-oxa-9-azabicyclo[3.3.1]nonane-9-carboxylate (160 mg, 0.26 mmol) was used instead of tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate. MS (ESI): mass calcd. for C₂₇H₃₀N₈O3, 514.2. m/z found, 515.3 [M+H]⁺. ¹H NMR (400 MHz, DMSO-d₆) δ 10.75 (s, 1H), 8.78-8.76 (m, 1H), 8.61-8.57 (m, 2H), 8.54 (t, J=6.0 Hz, 1H), 8.30 (s, 1H), 8.07-8.05 (m, 1H), 7.38 (s, 1H), 7.19 (dd, J=1.6, 7.2 Hz, 1H), 6.96 (s, 1H), 4.75-4.67 (m, 1H), 3.70-3.55 (m, 4H), 3.32-3.31 (m, 2H), 3.15-3.09 (m, 2H), 2.47 (s, 3H), 2.40-2.32 (m, 2H), 1.83-1.76 (m, 2H), 1.13 (t, J=7.2 Hz, 3H).

### Example 387: N-(2-methoxyethyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide

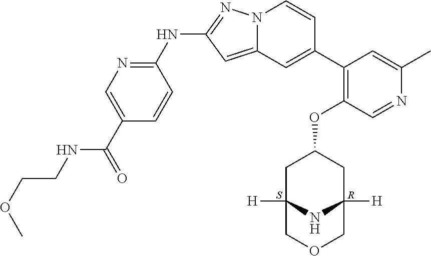

The title compound (10 mg, 39%) was prepared as described in Example 371 except 2-methoxyethanamine (15 mg, 0.21 mmol) was used instead of 1-amino-2-methylpropan-2-ol in Step A and HCl in 1,4-dioxane was used instead of TFA in Step B. MS (ESI): mass calcd. for C₂₉H₃₃N₇O₄, 543.3. m/z found, 544.2 [M+H]⁺. ¹H NMR (400 MHz, CDCl₃) δ 8.72 (d, J=2.0 Hz, 1H), 8.34 (d, J=7.2 Hz, 1H), 8.23 (s, 1H), 8.05 (dd, J=2.0, 8.8 Hz, 1H), 7.91 (s, 1H), 7.85 (s, 1H), 7.47 (d, J=8.8 Hz, 1H), 7.22 (s, 1H), 7.12 (dd, J=2.0, 7.2 Hz, 1H), 6.58 (s, 1H), 6.56 (t, J=5.2 Hz, 1H), 4.64-4.53 (m, 1H), 3.79-3.70 (m, 4H), 3.70-3.64 (m, 2H), 3.62-3.56 (m, 2H), 3.41 (s, 3H), 3.08-3.00 (m, 2H), 2.56 (s, 3H), 2.41-2.28 (m, 2H), 2.04-1.93 (m, 2H).

### Example 388: 6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]-N-tetrahydropyran-4-yl-pyridine-3-carboxamide

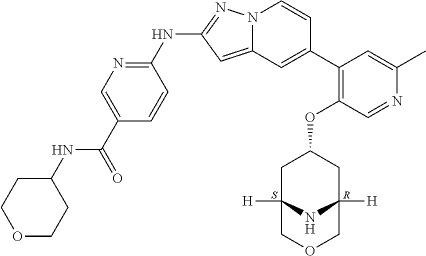

The title compound (23 mg, 90%) was prepared as described in Example 371 except tetrahydro-2H-pyran-4-amine (16 mg, 0.15 mmol) was used instead of 1-amino-2-methylpropan-2-ol in Step A and HCl in 1,4-dioxane was used instead of TFA in Step B. MS (ESI): mass calcd. for C₃₁H₃₅N₇O₄, 569.3. m/z found, 570.1 [M+H]⁺. ¹H NMR (400 MHz, DMSO-d₆) δ 10.24 (s, 1H), 8.74 (d, J=2.0 Hz, 1H), 8.56 (d, J=7.2 Hz, 1H), 8.30 (s, 1H), 8.22 (d, J=7.6 Hz, 1H), 8.12-7.93 (m, 2H), 7.38 (s, 1H), 7.35 (d, J=8.8 Hz, 1H), 7.12 (dd, J=1.6, 7.2 Hz, 1H), 6.86 (s, 1H), 4.74-4.61 (m, 1H), 4.08-3.95 (m, 1H), 3.94-3.82 (m, 2H), 3.61-3.55 (m, 2H), 3.54-3.49 (m, 2H), 3.43-3.41 (m, 2H), 2.97-2.88 (m, 2H), 2.46 (s, 3H), 2.37-2.23 (m, 2H), 1.83-1.72 (m, 2H), 1.71-1.51 (m, 4H).

### Example 389: N-(2-cyano-2-methyl-propyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide

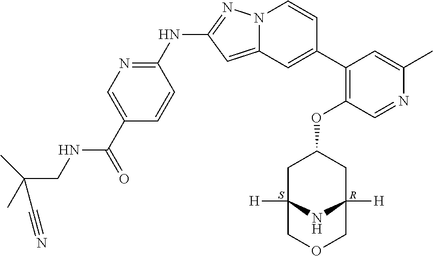

The title compound (40 mg, 44%) was prepared as described in Example 371 except 3-amino-2,2-dimethylpropanenitrile (67 mg, 0.68 mmol) was used instead of 1-amino-2-methylpropan-2-ol in Step A. MS (ESI): mass calcd. for C₃₁H₃₄N₈O₃, 566.3. m/z found, 567.2 [M+H]⁺. ¹H NMR (400 MHz, CDCl₃) δ 8.79 (d, J=2.0 Hz, 1H), 8.37 (d, J=7.2 Hz, 1H), 8.23 (s, 1H), 8.08 (dd, J=2.4, 8.8 Hz, 1H), 7.93 (s, 1H), 7.91-7.86 (m, 1H), 7.50 (d, J=8.8 Hz, 1H), 7.22 (s, 1H), 7.14 (dd, J=2.0, 7.2 Hz, 1H), 6.59 (s, 1H), 6.55 (t, J=1.2 Hz, 1H), 4.63-4.56 (m, 1H), 3.79-3.71 (m, 4H), 3.64 (d, J=6.8 Hz, 2H), 3.07-3.02 (m, 2H), 2.56 (s, 3H), 2.41-2.32 (m, 2H), 2.02-1.95 (m, 2H), 1.44 (s, 6H).

### Example 390: N-(2-hydroxy-2-methyl-propyl)-5-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyrazine-2-carboxamide

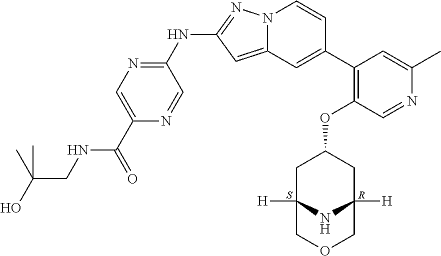

Step A: tert-butyl (1R,5S,7s)-7-((4-(2-((5-(methoxycarbonyl)pyrazin-2-yl)amino)pyrazolo[1,5-a]pyridin-5-yl)-6-methylpyridin-3-yl)oxy)-3-oxa-9-azabicyclo[3.3.1]nonane-9-carboxylate. The title compound (290 mg, 48%) was prepared as described in Example 116 Step A except methyl 5-chloropyrazine-2-carboxylate (122 mg, 0.71 mmol) was used instead of 4-bromo-1-(difluoromethyl)-1H-pyrazole.

Step B: 5-((5-(5-(((1R,5S,7s)-9-(tert-butoxycarbonyl)-3-oxa-9-azabicyclo[3.3.1]nonan-7-yl)oxy)-2-methylpyridin-4-yl)pyrazolo[1,5-a]pyridin-2-yl)amino)pyrazine-2-carboxylic acid. The title compound (180 mg, 68%) was prepared as described in Example 371, Step B except tert-butyl (1R,5S,7s)-7-((4-(2-((5-(methoxycarbonyl)pyrazin-2-yl)amino)pyrazolo[1,5-a]pyridin-5-yl)-6-methylpyridin-3-yl)oxy)-3-oxa-9-azabicyclo[3.3.1]nonane-9-carboxylate was used instead of tert-butyl (1R,5S,7s)-7-((4-(2-((5-(methoxycarbonyl)pyridin-2-yl)amino)pyrazolo[1,5-a]pyridin-5-yl)-6-methylpyridin-3-yl)oxy)-3-oxa-9-azabicyclo[3.3.1]nonane-9-carboxylate.

Step C: tert-butyl (1R,5S,7s)-7-((4-(2-((5-((2-hydroxy-2-methylpropyl)carbamoyl)pyrazin-2-yl)amino)pyrazolo[1,5-a]pyridin-5-yl)-6-methylpyridin-3-yl)oxy)-3-oxa-9-azabicyclo[3.3.1]nonane-9-carboxylate. The title compound (100 mg, 77%) was prepared as described in Example 371, Step A except 5-((5-(5-(((1R,5S,7s)-9-(tert-butoxycarbonyl)-3-oxa-9-azabicyclo[3.3.1]nonan-7-yl)oxy)-2-methylpyridin-4-yl)pyrazolo[1,5-a]pyridin-2-yl)amino)pyrazine-2-carboxylic acid was used instead of 6-((5-(5-(((1R,5S,7s)-9-(tert-butoxycarbonyl)-3-oxa-9-azabicyclo[3.3.1]nonan-7-yl)oxy)-2-methylpyridin-4-yl)pyrazolo[1,5-a]pyridin-2-yl)amino)nicotinic acid.

Step D: N-(2-hydroxy-2-methyl-propyl)-5-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyrazine-2-carboxamide. The title compound (36.9 mg, 42%) was prepared as described in Example 116, Step B except tert-butyl (1R,5S,7s)-7-((4-(2-((5-((2-hydroxy-2-methylpropyl)carbamoyl)pyrazin-2-yl)amino)pyrazolo[1,5-a]pyridin-5-yl)-6-methylpyridin-3-yl)oxy)-3-oxa-9-azabicyclo[3.3.1]nonane-9-carboxylate (100 mg, 0.15 mmol) was used instead of tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate. MS (ESI): mass calcd. for C₂₉H₃₄N₈O₄, 558.3. m/z found, 559.2 [M+H]⁺. ¹H NMR (400 MHz, DMSO-d₆) δ 10.82 (s, 1H), 8.80 (s, 1H), 8.64-8.57 (m, 2H), 8.30 (s, 1H), 8.16 (t, J=6.0 Hz, 1H), 8.05 (s, 1H), 7.38 (s, 1H), 7.18 (dd, J=1.6, 7.2 Hz, 1H), 6.97 (s, 1H), 4.73 (s, 1H), 4.71-4.62 (m, 1H), 3.61-3.44 (m, 4H), 3.28 (d, J=6.0 Hz, 2H), 2.96-2.91 (m, 2H), 2.46 (s, 3H), 2.34-2.25 (m, 2H), 1.69-1.62 (m, 2H), 1.12 (s, 6H).

### Example 391: 5-(5-(((1R,5S,7s)-3-oxa-9-azabicyclo[3.3.1]nonan-7-yl)oxy)-2-methylpyridin-4-yl)-N-(5-methylpyrimidin-2-yl)pyrazolo[1,5-a]pyridin-2-amine

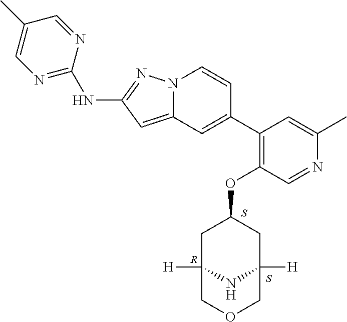

The title compound (51 mg, 41%) was prepared as described in Example 116, Step A except 5-(5-(((1R,5S,7s)-3-oxa-9-azabicyclo[3.3.1]nonan-7-yl)oxy)-2-methylpyridin-4-yl)pyrazolo[1,5-a]pyridin-2-amine (Example 405, 100 mg, 0.27 mmol) was used instead of Intermediate 9 and 2-chloro-5-methylpyrimidine (35 mg, 0.27 mmol) was used instead of 4-bromo-1-(difluoromethyl)-1H-pyrazole. MS (ESI): mass calcd. for C₂₅H₂₇N₇O₂, 457.2. m/z found, 458.1 [M+H]⁺. ¹H NMR (500 MHz, CD₃OD) δ 8.35-8.23 (m, 3H), 8.07 (s, 1H), 7.96 (dd, J=2.0, 0.9 Hz, 1H), 7.32 (s, 1H), 7.06 (dd, J=7.2, 2.0 Hz, 1H), 6.93 (s, 1H), 4.60-4.55 (m, 1H), 3.73-3.59 (m, 4H), 2.92 (d, J=7.3 Hz, 2H), 2.43 (s, 3H), 2.35-2.25 (m, 2H), 2.14 (s, 3H), 1.93-1.85 (m, 2H).

### Example 392: 5-[2-methyl-5-[[(1S,5R,7s)-3-oxa-9-azabicyclo[3.3.1]nonan-7-yl]oxy]-4-pyridyl]-N-pyrazin-2-yl-pyrazolo[1,5-a]pyridin-2-amine

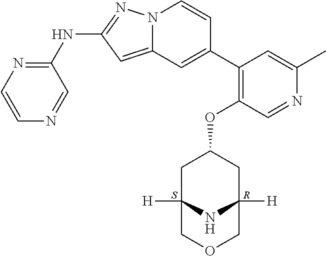

The title compound (140 mg, 63%) was prepared as described in Example 116, Step A-B except 2-bromopyrazine (307 mg, 1.93 mmol) was used instead of 4-bromo-1-(difluoromethyl)-1H-pyrazole in Step A. MS (ESI): mass calcd. for C₂₄H₂₅N₇O₂, 443.2. m/z found, 444.2 [M+H]⁺. ¹H NMR (400 MHz, DMSO-d₆) δ 10.30 (s, 1H), 8.63 (s, 1H), 8.57 (d, J=6.8 Hz, 1H), 8.30 (s, 1H), 8.23 (s, 1H), 8.05-7.97 (m, 2H), 7.37 (s, 1H), 7.13 (dd, J=1.2, 6.8 Hz, 1H), 6.88 (s, 1H), 4.73-4.62 (m, 1H), 3.62-3.55 (m, 2H), 3.55-3.48 (m, 2H), 3.00-2.90 (m, 2H), 2.46 (s, 3H), 2.36-2.24 (m, 2H), 1.73-1.62 (m, 2H)

### Example 393: 5-[2-methyl-5-[[(1R,5S,7s)-3-oxa-9-azabicyclo[3.3.1]nonan-7-yl]oxy]-4-pyridyl]-N-(5-methylsulfonyl-2-pyridyl)pyrazolo[1,5-a]pyridin-2-amine

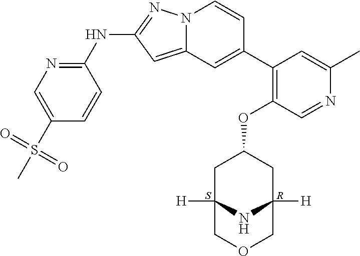

The title compound (35 mg, 74%) was prepared as described in Example 116, Step A-B except 2-bromo-5-(methylsulfonyl)pyridine (76 mg, 0.32 mmol) was used instead of 4-bromo-1-(difluoromethyl)-1H-pyrazole in Step A. MS (ESI): mass calcd. for C₂₆H₂₈N₆O₄S, 520.2. m/z found, 521.2 [M+H]⁺. ¹H NMR (400 MHz, DMSO-d₆) δ 10.65 (s, 1H), 8.69 (d, J=2.4 Hz, 1H), 8.58 (d, J=7.2 Hz, 1H), 8.30 (s, 1H), 8.09-8.03 (m, 2H), 7.44 (d, J=8.8 Hz, 1H), 7.39 (s, 1H), 7.17 (dd, J=1.6, 7.2 Hz, 1H), 6.91 (s, 1H), 4.75-4.66 (m, 1H), 3.69-3.62 (m, 2H), 3.61-3.54 (m, 2H), 3.23 (s, 3H), 3.12-3.05 (m, 2H), 2.47 (s, 3H), 2.41-2.29 (m, 2H), 1.82-1.71 (m, 2H)

### Example 394: 5-[2-methyl-5-[[(1R,5S,7s)-3-oxa-9-azabicyclo[3.3.1]nonan-7-yl]oxy]-4-pyridyl]-N-pyridazin-3-yl-pyrazolo[1,5-a]pyridin-2-amine

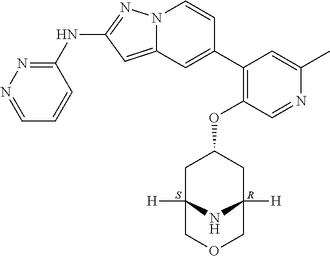

The title compound (41 mg, 71%) was prepared as described in Example 116, Step A-B except 3-bromopyridazine (171 mg, 1.07 mmol) was used instead of 4-bromo-1-(difluoromethyl)-1H-pyrazole in Step A. MS (ESI): mass calcd. for C₂₄H₂₅N₇O₂, 443.2. m/z found, 444.2 [M+H]⁺. ¹H NMR (400 MHz, DMSO-d₆) δ 10.18 (s, 1H), 8.72 (d, J=4.0 Hz, 1H), 8.56 (d, J=7.2 Hz, 1H), 8.30 (s, 1H), 8.07 (s, 1H), 7.66-7.58 (m, 1H), 7.55-7.48 (m, 1H), 7.39 (s, 1H), 7.13 (dd, J=1.2, 6.8 Hz, 1H), 6.91 (s, 1H), 4.79-4.61 (m, 1H), 3.71-3.63 (m, 2H), 3.63-3.55 (m, 2H), 3.11 (d, J=6.4 Hz, 2H), 2.47 (s, 3H), 2.40-2.31 (m, 2H), 1.85-1.74 (m, 2H).

### Example 395: 5-[2-methyl-5-[[(1R,5S,7s)-3-oxa-9-azabicyclo[3.3.1]nonan-7-yl]oxy]-4-pyridyl]-N-pyrimidin-4-yl-pyrazolo[1,5-a]pyridin-2-amine

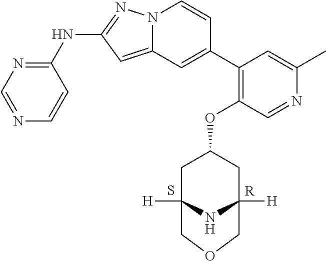

The title compound (10 mg, 21%) was prepared as described in Example 116, Step A-B except 4-chloropyrimidine hydrochloride (130 mg, 0.86 mmol) was used instead of 4-bromo-1-(difluoromethyl)-1H-pyrazole in Step A. MS (ESI): mass calcd. for C₂₄H₂₅N₇O₂, 443.2. m/z found, 444.2 [M+H]⁺. ¹H NMR (400 MHz, DMSO-d₆) δ 10.44 (s, 1H), 8.71 (s, 1H), 8.58 (d, J=6.8 Hz, 1H), 8.37 (d, J=6.0 Hz, 1H), 8.30 (s, 1H), 8.04 (s, 1H), 7.37 (s, 1H), 7.35-7.28 (m, 1H), 7.19-7.13 (m, 1H), 6.87 (s, 1H), 4.74-4.61 (m, 1H), 3.61-3.47 (m, 4H), 2.99-2.88 (m, 2H), 2.46 (s, 3H), 2.32-2.24 (m, 2H), 1.71-1.60 (m, 2H).

### Example 396: 5-[2-methyl-5-[[(1R,5S,7s)-3-oxa-9-azabicyclo[3.3.1]nonan-7-yl]oxy]-4-pyridyl]-N-(6-methylpyrimidin-4-yl)pyrazolo[1,5-a]pyridin-2-amine

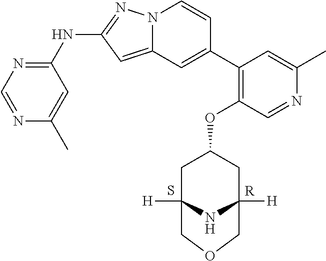

The title compound (25 mg, 89%) was prepared as described in Example 116, Step A-B except 4-chloro-6-methylpyrimidine (133 mg, 0.81 mmol) was used instead of 4-bromo-1-(difluoromethyl)-1H-pyrazole in Step A. MS (ESI): mass calcd. for C₂₅H₂₇N₇O₂, 457.2. m/z found, 458.1 [M+H]⁺. ¹H NMR (400 MHz, DMSO-d₆) δ 10.31 (s, 1H), 8.61-8.55 (m, 2H), 8.30 (s, 1H), 8.04-8.01 (m, 1H), 7.37 (s, 1H), 7.14 (dd, J=1.6, 6.8 Hz, 2H), 6.85 (s, 1H), 4.72-4.63 (m, 1H), 3.60-3.47 (m, 4H), 2.96-2.89 (m, 2H), 2.46 (s, 3H), 2.34 (s, 3H), 2.33-2.25 (m, 2H), 1.70-1.60 (m, 2H).

### Example 397: N-(5-fluoro-1-methyl-pyrazol-3-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine

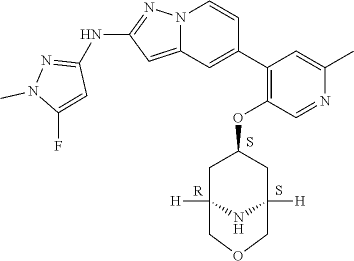

The title compound (113 mg, 67%) was prepared as described in Example 116, Step A-B except 3-bromo-5-fluoro-1-methyl-1h-pyrazole (81 mg, 0.45 mmol) was used instead of 4-bromo-1-(difluoromethyl)-1H-pyrazole in Step A. MS (ESI): mass calcd. for C₂₄H₂₆FN₇O₂, 463.2. m/z found, 464.3 [M+H]⁺. ¹H NMR (500 MHz, DMSO-d₆) δ 9.18 (s, 1H), 8.43 (d, J=7.2 Hz, 1H), 8.28 (s, 1H), 7.87 (dd, J=2.0, 0.9 Hz, 1H), 7.34 (s, 1H), 7.00 (dd, J=7.2, 2.0 Hz, 1H), 6.36 (d, J=0.9 Hz, 1H), 5.82 (d, J=5.5 Hz, 1H), 4.70-4.59 (m, 1H), 3.58 (d, J=0.9 Hz, 3H), 3.55 (dd, J=10.7, 2.4 Hz, 2H), 3.49 (d, J=10.8 Hz, 2H), 2.92 (d, J=8.7 Hz, 2H), 2.45 (s, 3H), 2.33-2.23 (m, 2H), 1.70-1.58 (m, 2H).

### Example 398: N-(3-fluoro-1-methyl-pyrazol-4-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine

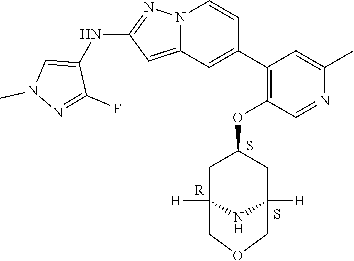

The title compound (54 mg, 60%) was prepared as described in Example 116, Step A-B except 4-bromo-3-fluoro-1-methyl-1h-pyrazole (85 mg, 0.48 mmol) was used instead of 4-bromo-1-(difluoromethyl)-1H-pyrazole in Step A. MS (ESI): mass calcd. for C₂₄H₂₆FN₇O₂, 463.2. m/z found, 464.2 [M+H]⁺. ¹H NMR (500 MHz, DMSO-d₆) δ 8.39 (d, J=7.0 Hz, 1H), 8.27 (s, 1H), 8.06 (s, 1H), 7.85-7.74 (m, 2H), 7.33 (s, 1H), 7.01-6.90 (m, 1H), 5.82 (s, 1H), 4.67-4.60 (m, 1H), 3.71 (s, 3H), 3.55 (d, J=10.5 Hz, 2H), 3.49 (d, J=10.6 Hz, 2H), 2.92 (d, J=8.5 Hz, 2H), 2.44 (s, 3H), 2.31-2.22 (m, 2H), 1.69-1.58 (m, 2H).

### Example 399: N-(4-fluoro-1-methyl-pyrazol-3-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine

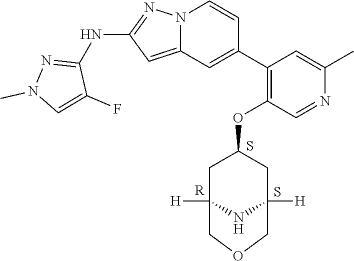

The title compound (66 mg, 67%) was prepared as described in Example 116, Step A-B except 3-bromo-4-fluoro-1-methyl-1H-pyrazole (85 mg, 0.48 mmol) was used instead of 4-bromo-1-(difluoromethyl)-1H-pyrazole in Step A. MS (ESI): mass calcd. for C₂₄H₂₆FN₇O₂, 463.2. m/z found, 464.2 [M+H]⁺. ¹H NMR (500 MHz, DMSO-d₆) δ 8.78 (s, 1H), 8.43-8.39 (m, 1H), 8.28 (s, 1H), 7.86 (dd, J=2.0, 0.9 Hz, 1H), 7.75 (d, J=4.6 Hz, 1H), 7.33 (s, 1H), 7.00 (dd, J=7.2, 2.0 Hz, 1H), 6.30 (d, J=0.8 Hz, 1H), 4.68-4.59 (m, 1H), 3.70 (s, 3H), 3.55 (dd, J=10.7, 2.3 Hz, 2H), 3.49 (d, J=10.8 Hz, 2H), 2.92 (d, J=8.6 Hz, 2H), 2.45 (s, 3H), 2.32-2.23 (m, 2H), 1.69-1.58 (m, 2H).

### Example 400: N-[3-methyl-5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]cyclopropanecarboxamide

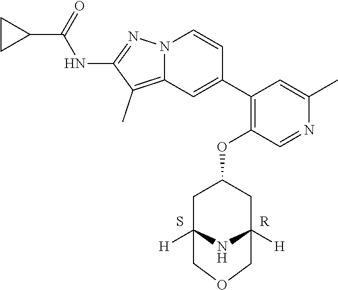

Step A: N-(5-(5-fluoro-2-methylpyridin-4-yl)-3-formylpyrazolo[1,5-a]pyridin-2-yl)cyclopropanecarboxamide. To a solution of N-(5-(5-fluoro-2-methylpyridin-4-yl)pyrazolo[1,5-a]pyridin-2-yl)cyclopropanecarboxamide (500 mg, 1.611 mmol) and DMF (15.594 mL, 0.944 g/mL, 201.398 mmol) was added POCl3 (0.15 mL, 1.65 g/mL, 1.611 mmol) at room temp. The solution turned yellow immediately. The solution was stirred for 2 h before quenching with 5 mL H₂O and then diluted with 50 mL DCM. The solution was washed with H₂O 4×. The organics were collected, dried over MgSO₄, filtered, and concentrated down. The crude was absorbed onto silica gel and purified via FCC with 0-10% MeOH/DCM. Took the material (355 mg, 65%) forward to the next step.

Step B: N-(5-(5-fluoro-2-methylpyridin-4-yl)-3-(hydroxymethyl)pyrazolo[1,5-a]pyridin-2-yl)cyclopropanecarboxamide. To a mixture of N-(5-(5-fluoro-2-methylpyridin-4-yl)-3-formylpyrazolo[1,5-a]pyridin-2-yl)cyclopropanecarboxamide (200 mg, 0.591 mmol) and THF (4.811 mL, 0.886 g/mL, 59.112 mmol) was added NaBH₄ (67.091 mg, 1.773 mmol) at room temp. The solution was stirred for 3 h, and the mixture turned into solution. The reaction was quenched with 5 mL H₂O and then diluted with 25 mL EtOAc. The layers were separated. The organics were washed 1× with 1M NaOH. The layers were separated. The organic layer was dried with MgSO₄, filtered, and concentrated down under vacuum. The crude (200 mg, 99%) was used in the next step without further purification.

Step C: N-(5-(5-fluoro-2-methylpyridin-4-yl)-3-methylpyrazolo[1,5-a]pyridin-2-yl)cyclopropanecarboxamide. To a residue of N-(5-(5-fluoro-2-methylpyridin-4-yl)-3-(hydroxymethyl)pyrazolo[1,5-a]pyridin-2-yl)cyclopropanecarboxamide (201.191 mg, 0.591 mmol) was added triethylsilane (687.345 mg, 5.911 mmol) and then added TFA (9.047 mL, 1.49 g/mL, 118.224 mmol). The reaction was stirred at room temp for 30 min. The crude was evaporated to dryness, diluted with EtOAc, and washed with NaHCO3 1×. The organics were collected, dried over MgSO₄, filtered, and concentrated down under vacuum. The crude material was purified via FCC with 5-10% MeOH-DCM to give 160 mg of the title compound (83%).

Step D: N-[3-methyl-5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]cyclopropanecarboxamide. To a 50 mL flask was added endo tert-butyl (1R,5S,7r)-7-hydroxy-3-oxa-9-azabicyclo[3.3.1]nonane-9-carboxylate (180.026 mg, 0.74 mmol) and DMA (1.484 mL, 0.941 g/mL, 16.032 mmol) and then NaH (60% dispersion in mineral oil) (49.324 mg, 1.233 mmol). The mixture was stirred for 5 min before injecting N-(5-(5-fluoro-2-methylpyridin-4-yl)-3-methylpyrazolo[1,5-a]pyridin-2-yl)cyclopropanecarboxamide (80 mg, 0.247 mmol) into the flask via syringe. The reaction was stirred at 80° C. in a heating block for 120 min. The crude was quenched with water and then diluted with EtOAc. The organics were extracted and concentrated down in the presence of silica gel. The crude was purified via FCC with 5-10% MeOH:DCM. The pure fractions were concentrated down to a clear oil. To this clear oil was added TFA (0.377 mL, 1.49 g/mL, 4.933 mmol) and DCM (0.948 mL, 1.326 g/mL, 14.798 mmol). The crude was evaporated in vacuo. The crude oil residue was dissolved with DCM/MeOH and concentrated down again. The crude was dissolved with DMSO and purified via ISCO prep. HPLC using 10-60% ACN-H₂O with 20 mM of NH₃OH as modifier. The fractions containing the desired product were collected and concentrated down to ˜30 mL under vacuo. The sample was lyophilized to give the title compound (63 mg, 57%). MS (ESI): mass calcd. for C₂₅H₂₉N₅O₃, 447.2. m/z found, 448.0 [M+H]⁺. ¹H NMR (500 MHz, DMSO-d₆) δ 10.28 (s, 1H), 8.50 (d, J=7.2 Hz, 1H), 8.29 (s, 1H), 7.97 (dd, J=2.0, 1.0 Hz, 1H), 7.40 (s, 1H), 7.16 (dd, J=7.3, 2.0 Hz, 1H), 4.67 (p, J=6.1 Hz, 1H), 3.64-3.48 (m, 4H), 3.00 (d, J=7.9 Hz, 2H), 2.47 (s, 3H), 2.31 (dt, J=14.1, 7.2 Hz, 2H), 2.13 (s, 3H), 1.89 (td, J=12.5, 12.1, 5.9 Hz, 1H), 1.77-1.66 (m, 2H), 0.81 (d, J=5.8 Hz, 4H).

### Example 401: 2-methyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridazin-3-one

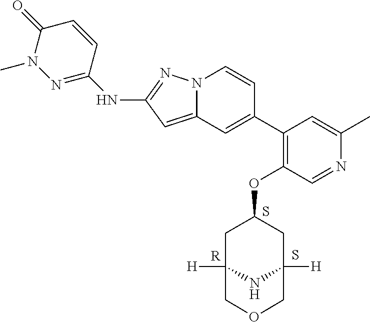

Step A: 6-((5-(5-fluoro-2-methylpyridin-4-yl)pyrazolo[1,5-a]pyridin-2-yl)amino)-2-methylpyridazin-3(2H)-one. The title compound (1.64 g, >100%) was prepared as described in Example 116, Step A except Intermediate 8 (1.02 g, 4.22 mmol) was used instead of Intermediate 9 and 6-bromo-2-methyl-3(2h)-pyridazinone (900 mg, 4.76 mmol) was used instead of 4-bromo-1-(difluoromethyl)-1H-pyrazole.

Step B: tert-butyl (1R,5S,7s)-7-((6-methyl-4-(2-((1-methyl-6-oxo-1,6-dihydropyridazin-3-yl)amino)pyrazolo[1,5-a]pyridin-5-yl)pyridin-3-yl)oxy)-3-oxa-9-azabicyclo[3.3.1]nonane-9-carboxylate. The title compound (616 mg, 71%) was prepared as described in Example 136 except 6-((5-(5-fluoro-2-methylpyridin-4-yl)pyrazolo[1,5-a]pyridin-2-yl)amino)-2-methylpyridazin-3(2H)-one (530 mg, 1.51 mmol) was used instead of Intermediate 6 and tert-butyl 7-hydroxy-3-oxa-9-azabicyclo[3.3.1]nonane-9-carboxylate was used instead of endo-7-hydroxy-3-oxa-9-azabicyclo[3.3.1]nonane hydrochloride.

Step C: 2-methyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridazin-3-one. The title compound (275 mg, 54%) was prepared as described in Example 116, Step B except tert-butyl (1R,5S,7s)-7-((6-methyl-4-(2-((1-methyl-6-oxo-1,6-dihydropyridazin-3-yl)amino)pyrazolo[1,5-a]pyridin-5-yl)pyridin-3-yl)oxy)-3-oxa-9-azabicyclo[3.3.1]nonane-9-carboxylate (616 mg, 1.07 mmol) was used instead of tert-butyl (1R,5S,7s)-7-((4-(2-((1-(difluoromethyl)-1H-pyrazol-4-yl)amino)pyrazolo[1,5-a]pyridin-5-yl)-6-methylpyridin-3-yl)oxy)-3-oxa-9-azabicyclo[3.3.1]nonane-9-carboxylate. MS (ESI): mass calcd. for C₂₅H₂₇N₇O₃, 473.2. m/z found, 474.1 [M+H]⁺. ¹H NMR (400 MHz, DMSO-d₆) δ 9.86 (s, 1H), 8.51 (dt, J=7.2, 0.9 Hz, 1H), 8.29 (s, 1H), 8.02 (dd, J=2.0, 0.9 Hz, 1H), 7.42-7.33 (m, 2H), 7.15 (dd, J=7.2, 2.0 Hz, 1H), 6.92 (d, J=9.7 Hz, 1H), 6.83 (d, J=0.8 Hz, 1H), 4.66 (p, J=6.4 Hz, 1H), 3.60 (s, 3H), 3.59-3.46 (m, 4H), 2.93 (d, J=7.9 Hz, 2H), 2.46 (s, 3H), 2.35-2.22 (m, 3H), 1.66 (ddd, J=13.8, 6.6, 2.5 Hz, 2H).

### Example 402: 5-[2-methyl-5-[[(1S,5R,7s)-3-oxa-9-azabicyclo[3.3.1]nonan-7-yl]oxy]-4-pyridyl]-N-pyrimidin-2-yl-pyrazolo[1,5-a]pyridin-2-amine

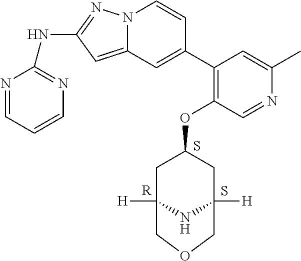

To tert-butyl (1R,5S,7s)-7-((4-(2-Aminopyrazolo[1,5-a]pyridin-5-yl)-6-methylpyridin-3-yl)oxy)-3-oxa-9-azabicyclo[3.3.1]nonane-9-carboxylate (Intermediate 9, 72 mg, 0.16 mmol) was added HCl in MeOH (1 mL, 1.25 M). The mixture was stirred for 2 h and concentrated. To this residue was added 1,4-dioxane (10 mL), then NaH (60% dispersion in mineral oil) (62 mg, 1.55 mmol) and 2-bromopyrimidine (25 mg, 0.16 mmol). The mixture was heated at 120° C. for 4 h. At 0° C., water was carefully added for quenching. Concentrated mixture. Water (20 mL) was added, then an extraction with ethyl acetate was performed (3×20 mL). Purification via FFC (10% MeOH/DCM) yields 25 mg of the title compound (36%). MS (ESI): mass calcd. for C₂₄H₂₅N₇O₂, 443.2. m/z found, 444.1 [M+H]⁺. ¹H NMR (400 MHz, CD₃OD) δ 8.52 (d, J=4.8 Hz, 2H), 8.49-8.37 (m, 1H), 8.16 (s, 1H), 8.06 (dd, J=2.0, 0.9 Hz, 1H), 7.40 (s, 1H), 7.17 (dd, J=7.3, 2.0 Hz, 1H), 7.07 (d, J=0.8 Hz, 1H), 6.87 (t, J=4.8 Hz, 1H), 4.74-4.61 (m, 1H), 3.87-3.66 (m, 4H), 2.97 (d, J=7.3 Hz, 2H), 2.52 (s, 3H), 2.44-2.31 (m, 2H), 2.02-1.92 (m, 2H).

### Example 403: 5-(5-(((1R,5S,7s)-3-oxa-9-azabicyclo[3.3.1]nonan-7-yl)oxy)-2-methylpyridin-4-yl)-N-(1-methyl-3-(trifluoromethyl)-1H-pyrazol-5-yl)pyrazolo[1,5-a]pyridin-2-amine

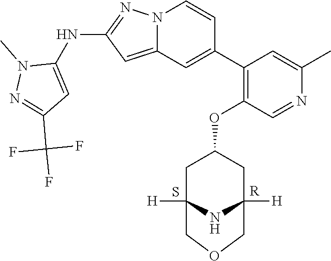

The title compound (36 mg, 25%) was prepared as described in Example 379, where the appropriate pyrazole isomer was obtained instead (see Example 379, Step B). MS (ESI): mass calcd. for C₂₅H₂₆F₃N₇O₂, 513.2. m/z found, 514.2 [M+H]⁺. ¹H NMR (400 MHz, DMSO-d₆) δ 9.31 (s, 1H), 8.56 (d, J=7.2 Hz, 1H), 8.29 (s, 1H), 7.96 (s, 1H), 7.37 (s, 1H), 7.07 (dd, J=1.6, 7.2 Hz, 1H), 6.74 (s, 1H), 6.14 (s, 1H), 4.80-4.59 (m, 1H), 3.82 (s, 3H), 3.60-3.55 (m, 2H), 3.54-3.49 (m, 2H), 2.94 (d, J=8.0 Hz, 2H), 2.45 (s, 3H), 2.38-2.21. ¹⁹F NMR (376 MHz, DMSO-d₆) δ −60.9 (s, 3F).

### Example 404: N-(5-fluoropyrimidin-2-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine

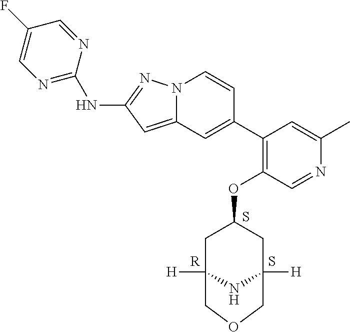

The title compound (41 mg, 32%) was prepared as described in Example 116, Step A except 5-(5-(((1R,5S,7s)-3-oxa-9-azabicyclo[3.3.1]nonan-7-yl)oxy)-2-methylpyridin-4-yl)pyrazolo[1,5-a]pyridin-2-amine (Example 405, 100 mg, 0.27 mmol) was used instead of Intermediate 9 and 2-chloro-5-fluoropyrimidine (36 mg, 0.27 mmol) was used instead of 4-bromo-1-(difluoromethyl)-1H-pyrazole. MS (ESI): mass calcd. for C₂₄H₂₄FN₇O₂, 461.2. m/z found, 462.1 [M+H]⁺. ¹H NMR (500 MHz, CD₃OD) δ 8.47 (s, 2H), 8.17 (s, 1H), 8.07 (d, J=2.1 Hz, 2H), 7.41 (s, 1H), 7.17 (dd, J=7.3, 2.0 Hz, 1H), 7.04 (s, 1H), 4.75-4-65 (m, 1H), 3.87-3.80 (m, 4H), 3.03-2.95 (m, 2H), 2.53 (s, 3H), 2.30-2.18 (m, 2H), 1.85-1.78 (m, 2H).

### Example 405: 5-(5-(((1R,5S,7s)-3-oxa-9-azabicyclo[3.3.1]nonan-7-yl)oxy)-2-methylpyridin-4-yl)pyrazolo[1,5-a]pyridin-2-amine

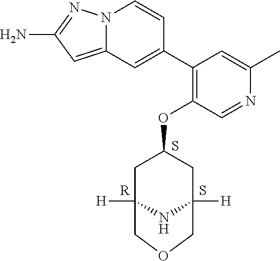

To a vial containing Intermediate 9 (140 mg, 0.30 mmol) was added TFA (0.81 mL, 10.5 mmol) at 23° C. The solution was stirred for 15 min. The solvent was removed in vacuo, then dissolved in DMSO to be purified by prep reverse phase basic HPLC (C18 50×100 mm) using 10-80% MeCN in water (20 mM NH₄OH). Fractions containing the product were pooled together and concentrated in vacuo. The resulting oil was dissolved in 1:1 MeCN:water and lyophilized to yield the title compound. MS (ESI): mass calcd. for C₂₀H₂₃N₅O₂, 365.2. m/z found, 366.2 [M+H]⁺. ¹H NMR (500 MHz, DMSO-d₆) δ 8.29-8.21 (m, 2H), 7.73 (dd, J=2.0, 0.9 Hz, 1H), 7.31 (s, 1H), 6.86 (dd, J=7.2, 2.1 Hz, 1H), 5.66 (d, J=0.8 Hz, 1H), 5.29 (s, 2H), 4.66-4.56 (m, 1H), 3.55 (dd, J=10.7, 2.4 Hz, 2H), 3.49 (d, J=10.5 Hz, 2H), 2.92 (d, J=8.7 Hz, 2H), 2.44 (s, 3H), 2.32-2.23 (m, 2H), 1.67-1.59 (m, 2H).

### Example 406: 5-[2-methyl-5-[[(1S,5R,7s)-9-methyl-3-oxa-9-azabicyclo[3.3.1]nonan-7-yl]oxy]-4-pyridyl]-N-[6-(trifluoromethyl)-2-pyridyl]pyrazolo[1,5-a]pyridin-2-amine

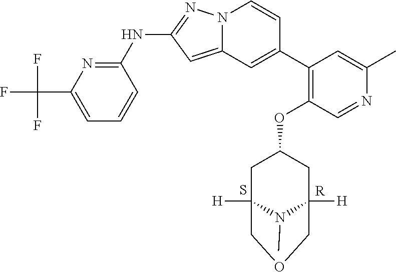

The title compound (43 mg, 41%) was prepared as described in Example 116, Step C except 5-[2-methyl-5-[[(1S,5R,7s)-3-oxa-9-azabicyclo[3.3.1]nonan-7-yl]oxy]-4-pyridyl]-N-[6-(trifluoromethyl)-2-pyridyl]pyrazolo[1,5-a]pyridin-2-amine (Example 366, 100 mg, 0.20 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₇H₂₇F₃N₆O₂, 524.2. m/z found, 525.3 [M+H]⁺. ¹H NMR (400 MHz, DMSO-d₆) δ 10.36 (s, 1H), 8.52 (d, J=7.2 Hz, 1H), 8.31 (s, 1H), 8.19 (s, 1H), 7.92-7.82 (m, 1H), 7.52 (d, J=8.4 Hz, 1H), 7.44 (s, 1H), 7.29-7.20 (m, 2H), 6.92 (s, 1H), 4.76-4.66 (m, 1H), 3.81 (d, J=10.0 Hz, 2H), 3.56 (d, J=10.8 Hz, 2H), 2.67-2.60 (m, 2H), 2.46 (s, 3H), 2.44-2.35 (m, 5H), 1.64 (d, J=15.2 Hz, 2H) ¹⁹F NMR (376 MHz, DMSO-d₆) δ −67.15 (s, 3F).

### Example 407: N-methyl-6-[[5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-yl]amino]pyridine-2-carboxamide

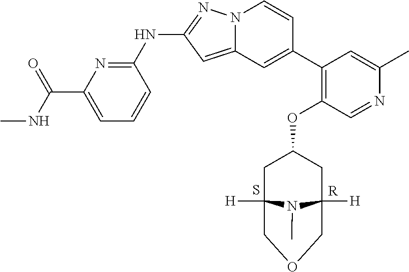

The title compound (25 mg, 12%) was prepared as described in Example 116, Step C except N-methyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-2-carboxamide (Example 367, 200 mg, 0.40 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₈H₃₁N₇O₃, 513.2. m/z found, 514.3 [M+H]⁺. ¹H NMR (400 MHz, DMSO-d₆) δ 10.00 (s, 1H), 8.52 (d, J=7.2 Hz, 1H), 8.23 (d, J=1.2 Hz, 1H), 8.20 (s, 1H), 8.15 (q, J=4.4 Hz, 1H), 7.85-7.75 (m, 1H), 7.49 (d, J=8.4 Hz, 1H), 7.45 (d, J=7.2 Hz, 1H), 7.42 (s, 1H), 7.23 (dd, J=2.0, 7.2 Hz, 1H), 6.80 (s, 1H), 4.77-4.65 (m, 1H), 3.85-3.76 (m, 2H), 3.60-3.51 (m, 2H), 2.91 (d, J=5.2 Hz, 3H), 2.67-2.61 (m, 2H), 2.46 (s, 3H), 2.44-2.32 (m, 5H), 1.71-1.60 (m, 2H)

### Example 408: 5-[2-methyl-5-[[(1S,5R,7s)-9-methyl-3-oxa-9-azabicyclo[3.3.1]nonan-7-yl]oxy]-4-pyridyl]-N-pyridazin-4-yl-pyrazolo[1,5-a]pyridin-2-amine

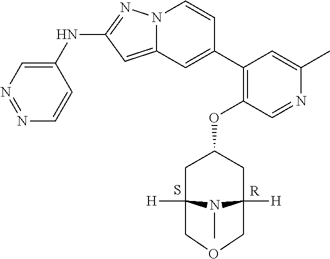

The title compound (21 mg, 16%) was prepared as described in Example 116, Step C except 5-[2-methyl-5-[[(1S,5R,7s)-3-oxa-9-azabicyclo[3.3.1]nonan-7-yl]oxy]-4-pyridyl]-N-pyridazin-4-yl-pyrazolo[1,5-a]pyridin-2-amine (Example 368, 170 mg, 0.27 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₅H₂₇N₇O₂, 457.2. m/z found, 458.3 [M+H]⁺. ¹H NMR (400 MHz, DMSO-d₆) δ 9.99 (br. s., 1H), 9.18 (d, J=2.4 Hz, 1H), 8.84 (d, J=6.0 Hz, 1H), 8.61 (d, J=7.2 Hz, 1H), 8.24-8.15 (m, 2H), 7.87 (dd, J=2.8, 6.0 Hz, 1H), 7.41 (s, 1H), 7.25 (dd, J=1.6, 7.2 Hz, 1H), 6.23 (s, 1H), 4.80-4.65 (m, 1H), 3.84-3.76 (m, 2H), 3.53-3.47 (m, 2H), 2.66-2.60 (m, 2H), 2.46 (s, 3H), 2.44-2.30 (m, 5H), 1.69-1.56 (m, 2H)

### Example 409: N-methyl-2-[[5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-yl]amino]pyridine-4-carboxamide

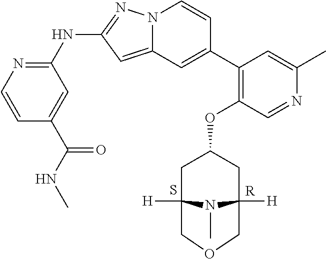

The title compound (18 mg, 23%) was prepared as described in Example 116 except 2-bromo-N-methylisonicotinamide (398 mg, 1.85 mmol) was used instead of 4-bromo-1-(difluoromethyl)-1H-pyrazole in Step A. MS (ESI): mass calcd. for C₂₈H₃₁N₇O₃, 513.2. m/z found, 514.2 [M+H]⁺. ¹H NMR (400 MHz, DMSO-d₆) δ 9.99 (s, 1H), 8.64-8.56 (m, 1H), 8.50 (d, J=7.2 Hz, 1H), 8.31 (d, J=5.2 Hz, 1H), 8.19 (s, 1H), 8.15 (s, 1H), 7.63 (s, 1H), 7.39 (s, 1H), 7.19 (d, J=7.6 Hz, 1H), 7.11 (d, J=5.2 Hz, 1H), 6.85 (s, 1H), 4.74-4.63 (m, 1H), 3.80 (d, J=10.4 Hz, 2H), 3.51 (d, J=10.4 Hz, 2H), 2.79 (d, J=4.0 Hz, 3H), 2.67-2.60 (m, 2H), 2.46 (s, 3H), 2.42-2.33 (m, 5H), 1.69-1.58 (m, 2H).

### Example 410: 5-[2-methyl-5-[[(1R,5S,7s)-9-methyl-3-oxa-9-azabicyclo[3.3.1]nonan-7-yl]oxy]-4-pyridyl]-N-(2-methylpyrimidin-4-yl)pyrazolo[1,5-a]pyridin-2-amine

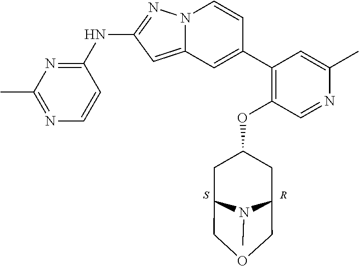

The title compound (30 mg, 41%) was prepared as described in Example 116, Step C except 5-[2-methyl-5-[[(1S,5R,7s)-3-oxa-9-azabicyclo[3.3.1]nonan-7-yl]oxy]-4-pyridyl]-N-(2-methylpyrimidin-4-yl)pyrazolo[1,5-a]pyridin-2-amine (Example 372, 70 mg, 0.15 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₆H₂₉N₇O₂, 471.2. m/z found, 472.3 [M+H]⁺. ¹H NMR (400 MHz, CD₃OD) δ 8.38 (d, J=7.2 Hz, 1H), 8.22-8.17 (m, 2H), 8.10 (s, 1H), 7.41 (s, 1H), 7.22 (dd, J=2.0, 7.2 Hz, 1H), 7.17 (d, J=4.8 Hz, 1H), 6.84 (s, 1H), 4.73-4.67 (m, 1H), 3.95 (d, J=10.0 Hz, 2H), 3.68 (d, J=10.8 Hz, 2H), 2.70 (d, J=5.6 Hz, 2H), 2.55 (s, 3H), 2.53-2.46 (m, 8H), 1.81 (d, J=16.0 Hz, 2H).

### Example 411: 5-[2-methyl-5-[[(1S,5R,7s)-9-methyl-3-oxa-9-azabicyclo[3.3.1]nonan-7-yl]oxy]-4-pyridyl]-N-[5-(trifluoromethyl)-2-pyridyl]pyrazolo[1,5-a]pyridin-2-amine

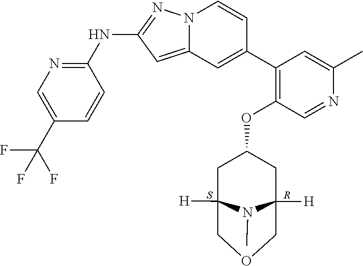

The title compound (30 mg, 56%) was prepared as described in Example 116, Step C except 5-[2-methyl-5-[[(1S,5R,7s)-3-oxa-9-azabicyclo[3.3.1]nonan-7-yl]oxy]-4-pyridyl]-N-[5-(trifluoromethyl)-2-pyridyl]pyrazolo[1,5-a]pyridin-2-amine (Example 374, 50 mg, 0.10 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₇H₂₇F₃N₆O₂, 524.2. m/z found, 525.3 [M+H]⁺. ¹H NMR (400 MHz, DMSO-d₆) δ 10.44 (s, 1H), 8.58 (s, 1H), 8.54 (d, J=7.2 Hz, 1H), 8.22-8.19 (m, 2H), 7.96 (dd, J=2.4, 8.8 Hz, 1H), 7.46 (d, J=8.8 Hz, 1H), 7.40 (s, 1H), 7.24 (dd, J=2.0, 7.2 Hz, 1H), 6.86 (s, 1H), 4.73-4.67 (m, 1H), 3.84-3.77 (m, 2H), 3.51 (d, J=10.6 Hz, 2H), 2.63 (d, J=6.0 Hz, 2H), 2.46 (s, 3H), 2.43-2.36 (m, 5H), 1.64 (d, J=14.8 Hz, 2H). ¹⁹F NMR (376 MHz, DMSO-d₆) δ −59.59 (s, 3F).

### Example 412: N-(3-fluoro-1-methyl-pyrazol-4-yl)-5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-amine

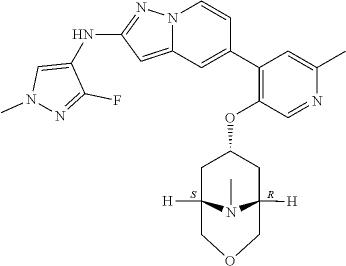

The title compound (867 mg, 46%) was prepared as described in Example 116, Step C except N-(3-fluoro-1-methyl-pyrazol-4-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine (Example 398, 1.75 g, 3.78 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₅H₂₈FN₇O₂, 477.2. m/z found, 478.2 [M+H]⁺. ¹H NMR (400 MHz, CDCl₃) δ 8.22 (d, J=7.2 Hz, 1H), 8.16 (s, 1H), 7.86 (d, J=0.8 Hz, 1H), 7.58 (d, J=2.0 Hz, 1H), 7.20 (s, 1H), 7.03 (dd, J=2.0, 7.2 Hz, 1H), 5.82 (s, 1H), 5.56 (s, 1H), 4.62-4.56 (m, 1H), 4.02-3.92 (m, 2H), 3.78 (s, 3H), 3.68-3.57 (m, 2H), 2.75-2.66 (m, 2H), 2.54 (s, 3H), 2.53 (s, 3H), 2.48-2.35 (m, 2H), 1.86-1.76 (m, 2H). ¹⁹F NMR (376 MHz, CDCl₃) δ −138.46 (s, 1F).

### Example 413: 5-[2-methyl-5-[[(1R,5S,7s)-9-methyl-3-oxa-9-azabicyclo[3.3.1]nonan-7-yl]oxy]-4-pyridyl]-N-[4-(trifluoromethyl)-2-pyridyl]pyrazolo[1,5-a]pyridin-2-amine

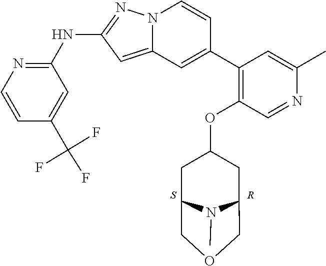

The title compound (21 mg, 40%) was prepared as described in Example 116, Step C except 5-[2-methyl-5-[[(1S,5R,7s)-3-oxa-9-azabicyclo[3.3.1]nonan-7-yl]oxy]-4-pyridyl]-N-[4-(trifluoromethyl)-2-pyridyl]pyrazolo[1,5-a]pyridin-2-amine (Example 378, 50 mg, 0.10 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₇H₂₇F₃N₆O₂, 524.2. m/z found, 525.3 [M+H]⁺. ¹H NMR (400 MHz, DMSO-d₆) δ 10.30 (s, 1H), 8.55 (d, J=7.2 Hz, 1H), 8.48 (d, J=5.2 Hz, 1H), 8.25-8.14 (m, 2H), 7.68 (s, 1H), 7.40 (s, 1H), 7.22 (d, J=7.2 Hz, 1H), 7.10 (d, J=5.2 Hz, 1H), 6.81 (s, 1H), 4.75-4.61 (m, 1H), 3.80 (d, J=10.4 Hz, 2H), 3.52 (d, J=10.8 Hz, 2H), 2.74-2.61 (m, 2H), 2.46 (s, 3H), 2.41 (s, 3H), 2.39-2.28 (m, 2H), 1.65 (d, J=14.8 Hz, 2H). ¹⁹F NMR (376 MHz, DMSO-d₆) δ −63.8 (s, 3F).

### Example 414: 5-[2-methyl-5-[[(1R,5S,7s)-9-methyl-3-oxa-9-azabicyclo[3.3.1]nonan-7-yl]oxy]-4-pyridyl]-N-(1-methyltriazol-4-yl)pyrazolo[1,5-a]pyridin-2-amine

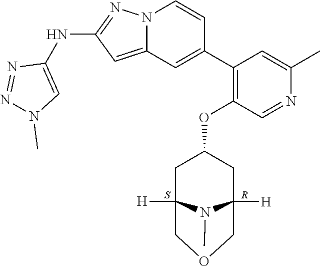

The title compound (18 mg, 36%) was prepared as described in Example 116, Step C except 5-[2-methyl-5-[[(1R,5S,7s)-3-oxa-9-azabicyclo[3.3.1]nonan-7-yl]oxy]-4-pyridyl]-N-(1-methyltriazol-4-yl)pyrazolo[1,5-a]pyridin-2-amine (Example 380, 50 mg, 0.10 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₄H₂₈N₈O₂, 460.2. m/z found, 461.3 [M+H]⁺. ¹H NMR (400 MHz, DMSO-d₆) δ 9.49 (s, 1H), 8.46 (d, J=7.2 Hz, 1H), 8.18 (s, 1H), 8.08 (s, 1H), 7.65 (s, 1H), 7.38 (s, 1H), 7.12 (dd, J=2.0, 7.2 Hz, 1H), 6.22 (s, 1H), 4.77-4.60 (m, 1H), 4.04 (s, 3H), 3.86-3.71 (m, 2H), 3.60-3.47 (m, 2H), 2.69-2.58 (m, 2H), 2.45 (s, 3H), 2.43-2.34 (m, 5H), 1.72-1.56 (m, 2H).

### Example 415: 5-[2-methyl-5-[[(1S,5R,7s)-9-methyl-3-oxa-9-azabicyclo[3.3.1]nonan-7-yl]oxy]-4-pyridyl]-N-pyrimidin-5-yl-pyrazolo[1,5-a]pyridin-2-amine

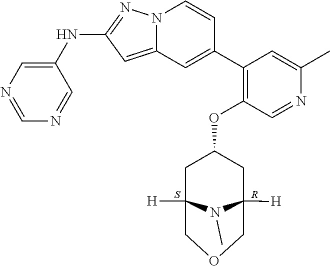

The title compound (8 mg, 25%) was prepared as described in Example 116 except 5-bromopyrimidine (123 mg, 0.78 mmol) was used instead of 4-bromo-1-(difluoromethyl)-1H-pyrazole in Step A. MS (ESI): mass calcd. for C₂₅H₂₇N₇O₂, 457.2. m/z found, 458.2 [M+H]⁺. ¹H NMR (400 MHz, CD₃CN) δ 9.01 (s, 2H), 8.67 (s, 1H), 8.38 (d, J=7.2 Hz, 1H), 8.18 (s, 1H), 8.02 (s, 1H), 7.60 (s, 1H), 7.31 (s, 1H), 7.16 (dd, J=2.0, 7.2 Hz, 1H), 6.12 (s, 1H), 4.74-4.65 (m, 1H), 4.04-3.87 (m, 2H), 3.66-3.51 (m, 2H), 2.85-2.73 (m, 2H), 2.59-2.42 (m, 8H), 1.88-1.79 (m, 2H).

### Example 416: 5-[2-methyl-5-[[(1S,5R,7s)-9-methyl-3-oxa-9-azabicyclo[3.3.1]nonan-7-yl]oxy]-4-pyridyl]-N-(5-methylpyrazin-2-yl)pyrazolo[1,5-a]pyridin-2-amine

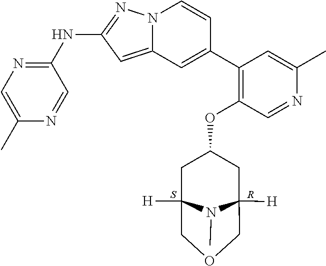

The title compound (20 mg, 48%) was prepared as described in Example 116, Step C except 5-[2-methyl-5-[[(1S,5R,7s)-3-oxa-9-azabicyclo[3.3.1]nonan-7-yl]oxy]-4-pyridyl]-N-(5-methylpyrazin-2-yl)pyrazolo[1,5-a]pyridin-2-amine (Example 381, 40 mg, 0.09 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₆H₂₉N₇O₂, 471.2. m/z found, 472.3 [M+H]⁺. ¹H NMR (400 MHz, DMSO-d₆) δ 10.07 (s, 1H), 8.57 (d, J=1.2 Hz, 1H), 8.52 (d, J=7.2 Hz, 1H), 8.19 (s, 1H), 8.17 (d, J=1.2 Hz, 1H), 8.12 (s, 1H), 7.39 (s, 1H), 7.20 (dd, J=2.0, 7.2 Hz, 1H), 6.77 (s, 1H), 4.77-4.63 (m, 1H), 3.87-3.75 (m, 2H), 3.57-3.46 (m, 2H), 2.63 (d, J=6.4 Hz, 2H), 2.46 (s, 3H), 2.44-2.34 (m, 8H), 1.69-1.58 (m, 2H).

### Example 417: N-(2,6-dimethylpyrimidin-4-yl)-5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-amine

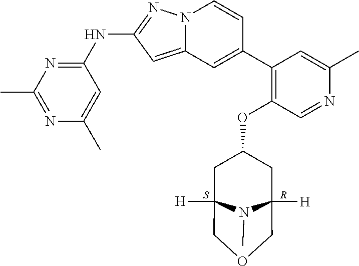

The title compound (37 mg, 36%) was prepared as described in Example 116, Step C except N-(2,6-dimethylpyrimidin-4-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine (Example 382, 100 mg, 0.21 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₇H₃₁N₇O₂, 485.3. m/z found, 486.2 [M+H]⁺. ¹H NMR (400 MHz, DMSO-d₆) δ 10.20 (s, 1H), 8.54 (d, J=7.2 Hz, 1H), 8.21 (s, 1H), 8.19 (s, 1H), 7.41 (s, 1H), 7.24 (dd, J=1.6, 7.2 Hz, 1H), 7.03 (br. s, 1H), 6.81 (br. s, 1H), 4.75-4.65 (m, 1H), 3.85-3.75 (m, 2H), 3.58-3.48 (m, 2H), 2.66-2.61 (m, 2H), 2.48-2.44 (m, 6H), 2.44-2.36 (m, 5H), 2.30 (s, 3H), 1.69-1.59 (m, 2H).

### Example 418: 5-[2-methyl-5-[[(1S,5R,7s)-9-methyl-3-oxa-9-azabicyclo[3.3.1]nonan-7-yl]oxy]-4-pyridyl]-N-pyrazin-2-yl-pyrazolo[1,5-a]pyridin-2-amine

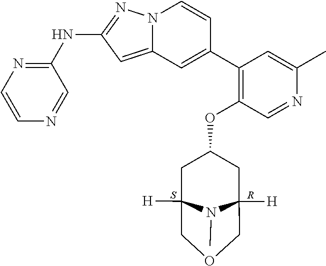

The title compound (48 mg, 51%) was prepared as described in Example 116, Step C except 5-[2-methyl-5-[[(1S,5R,7s)-3-oxa-9-azabicyclo[3.3.1]nonan-7-yl]oxy]-4-pyridyl]-N-pyrazin-2-yl-pyrazolo[1,5-a]pyridin-2-amine (Example 392, 90 mg, 0.20 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₅H₂₇N₇O₂, 457.2. m/z found, 458.3 [M+H]⁺. ¹H NMR (400 MHz, DMSO-d₆) δ 10.27 (s, 1H), 8.65 (d, J=1.2 Hz, 1H), 8.54 (d, J=7.6 Hz, 1H), 8.23 (dd, J=1.2 Hz, 2.4 Hz, 1H), 8.21-8.16 (m, 2H), 8.01 (d, J=2.8 Hz, 1H), 7.39 (s, 1H), 7.22 (dd, J=2.0, 7.2 Hz, 1H), 6.84 (s, 1H), 4.74-4.66 (m, 1H), 3.85-3.75 (m, 2H), 3.51 (d, J=10.8 Hz, 2H), 2.66-2.60 (m, 2H), 2.46 (s, 3H), 2.43-2.35 (m, 5H), 1.64 (d, J=15.2 Hz, 2H).

### Example 419: 5-[2-methyl-5-[[(1R,5S,7s)-9-methyl-3-oxa-9-azabicyclo[3.3.1]nonan-7-yl]oxy]-4-pyridyl]-N-pyridazin-3-yl-pyrazolo[1,5-a]pyridin-2-amine

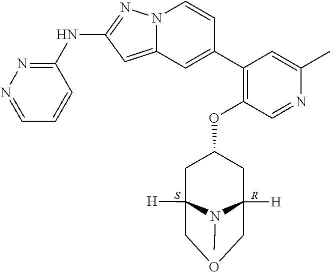

The title compound (33 mg, 6%) was prepared as described in Example 116, Step C except 5-[2-methyl-5-[[(1R,5S,7s)-3-oxa-9-azabicyclo[3.3.1]nonan-7-yl]oxy]-4-pyridyl]-N-pyridazin-3-yl-pyrazolo[1,5-a]pyridin-2-amine (Example 394, 500 mg, 1.13 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₅H₂₇N₇O₂, 457.2. m/z found, 458.3 [M+H]⁺. ¹H NMR (400 MHz, DMSO-d₆) δ 10.17 (s, 1H), 8.72 (d, J=4.4 Hz, 1H), 8.53 (d, J=7.2 Hz, 1H), 8.24 (s, 1H), 8.20 (s, 1H), 7.66-7.58 (m, 1H), 7.55-7.48 (m, 1H), 7.40 (s, 1H), 7.21 (dd, J=1.2, 7.2 Hz, 1H), 6.88 (s, 1H), 4.75-4.66 (m, 1H), 3.81 (d, J=10.4 Hz, 2H), 3.52 (d, J=10.8 Hz, 2H), 2.71-2.60 (m, 2H), 2.46 (s, 3H), 2.44-2.32 (m, 5H), 1.65 (d, J=14.8 Hz, 2H).

### Example 420: 5-[2-methyl-5-[[(1R,5S,7s)-9-methyl-3-oxa-9-azabicyclo[3.3.1]nonan-7-yl]oxy]-4-pyridyl]-N-pyrimidin-4-yl-pyrazolo[1,5-a]pyridin-2-amine

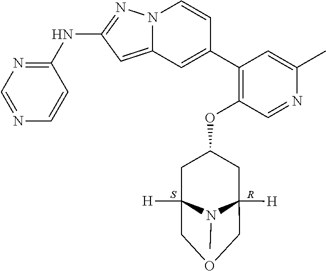

The title compound (11 mg, 5%) was prepared as described in Example 116, Step C except 5-[2-methyl-5-[[(1R,5S,7s)-3-oxa-9-azabicyclo[3.3.1]nonan-7-yl]oxy]-4-pyridyl]-N-pyrimidin-4-yl-pyrazolo[1,5-a]pyridin-2-amine (Example 395, 200 mg, 0.45 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₅H₂₇N₇O₂, 457.2. m/z found, 458.3 [M+H]⁺. ¹H NMR (400 MHz, DMSO-d₆) δ 10.42 (s, 1H), 8.71 (s, 1H), 8.55 (d, J=7.2 Hz, 1H), 8.37 (d, J=6.0 Hz, 1H), 8.20 (s, 2H), 7.39 (s, 1H), 7.36-7.28 (m, 1H), 7.25 (dd, J=2.0, 7.2 Hz, 1H), 6.82 (s, 1H), 4.74-4.66 (m, 1H), 3.84-3.75 (m, 2H), 3.58-3.43 (m, 2H), 2.66-2.60 (m, 2H), 2.46 (s, 3H), 2.45-2.34 (m, 6H), 1.68-1.59 (m, 2H).

### Example 421: 5-[2-methyl-5-[[(1R,5S,7s)-9-methyl-3-oxa-9-azabicyclo[3.3.1]nonan-7-yl]oxy]-4-pyridyl]-N-(6-methylpyrimidin-4-yl)pyrazolo[1,5-a]pyridin-2-amine

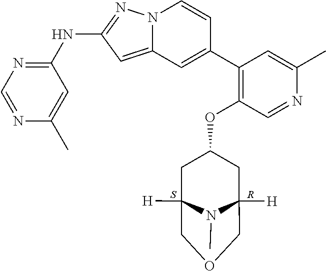

The title compound (47 mg, 15%) was prepared as described in Example 116, Step C except 5-[2-methyl-5-[[(1R,5S,7s)-3-oxa-9-azabicyclo[3.3.1]nonan-7-yl]oxy]-4-pyridyl]-N-(6-methylpyrimidin-4-yl)pyrazolo[1,5-a]pyridin-2-amine (Example 396, 300 mg, 0.66 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₆H₂₉N₇O₂, 471.2. m/z found, 472.3 [M+H]⁺. ¹H NMR (400 MHz, DMSO-d₆) δ 10.30 (s, 1H), 8.59 (s, 1H), 8.55 (d, J=7.2 Hz, 1H), 8.20 (s, 1H), 8.19-8.16 (m, 1H), 7.39 (s, 1H), 7.24 (dd, J=1.6, 6.8 Hz, 1H), 7.16 (s, 1H), 6.81 (s, 1H), 4.77-4.62 (m, 1H), 3.88-3.74 (m, 2H), 3.59-3.46 (m, 2H), 2.76-2.60 (m, 2H), 2.46 (s, 3H), 2.45-2.36 (m, 5H), 2.34 (s, 3H), 1.74-1.57 (m, 2H).

### Example 422: 2,2,2-trifluoro-1-[1-methyl-4-[[5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-yl]amino]pyrazol-3-yl]ethanol

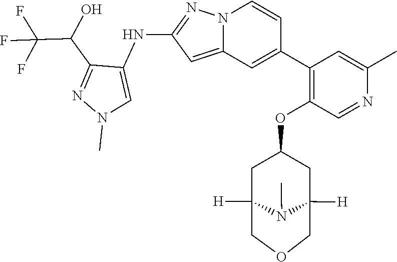 [SMILES: Cc1cc(-c2ccn3nc(Nc4cn(C)nc4C(O)C(F)(F)F)cc3c2)c(O[C@H]2C[C@H]3COC[C@@H](C2)N3C)cn1]

Step A: 1-(4-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-1-methyl-1H-pyrazol-3-yl)-2,2,2-trifluoroethan-1-one. A portion of the crude mixture (˜80 mg) from Example 123 was found to contain both the title compound and 7-endo-5-[2-methyl-5-[[(1S,5R)-3-oxa-9-azabicyclo[3.3.1]nonan-7-yl]oxy]-4-pyridyl]-N-(1-methylpyrazol-4-yl)pyrazolo[1,5-a]pyridin-2-amine (Example 123).

Step B: 2,2,2-trifluoro-1-[1-methyl-4-[[5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-yl]amino]pyrazol-3-yl]ethanol. The crude mixture from Step A (˜80 mg) was methylated using conditions found in Example 116, Step C (the crude mixture from Example 422, Step A was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine) to give the title compound (6.5 mg, 6%). MS (ESI): mass calcd. for C₂₇H₃₀F₃N₇O₃, 557.2. m/z found, 558.2 [M+H]⁺. ¹H NMR (500 MHz, DMSO-d₆) δ 8.44 (dd, J=7.2, 0.9 Hz, 1H), 8.21 (s, 1H), 8.07 (s, 1H), 7.87 (d, J=0.8 Hz, 1H), 7.75 (d, J=1.8 Hz, 1H), 7.48 (d, J=0.8 Hz, 1H), 7.25 (s, 1H), 7.19 (dd, J=7.2, 2.0 Hz, 1H), 6.79 (s, 1H), 5.57 (d, J=7.4 Hz, 1H), 4.66 (s, 1H), 3.81 (s, 3H), 3.73 (d, J=10.7 Hz, 2H), 3.39 (t, J=10.3 Hz, 2H), 2.63 (t, J=1.9 Hz, 2H), 2.46 (s, 3H), 2.41-2.34 (m, 5H), 1.63 (t, J=13.6 Hz, 2H).

### Example 423: 5-[2-methyl-5-[[(1S,5R,7s)-9-methyl-3-oxa-9-azabicyclo[3.3.1]nonan-7-yl]oxy]-4-pyridyl]-N-(1-methylpyrazol-3-yl)pyrazolo[1,5-a]pyridin-2-amine

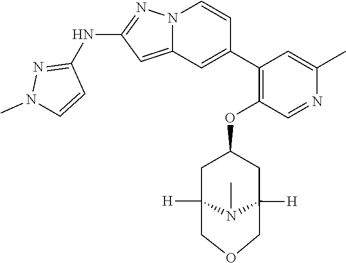

The title compound (59 mg, 55%) was prepared as described in Example 116, Step C except endo-5-(5-(((1R,5S,7s)-3-oxa-9-azabicyclo[3.3.1]nonan-7-yl)oxy)-2-methylpyridin-4-yl)-N-(1-methyl-1H-pyrazol-3-yl)pyrazolo[1,5-a]pyridin-2-amine (Example 125, 105 mg, 0.24 mmol) was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine. MS (ESI): mass calcd. for C₂₅H₂₉N₇O₂, 459.2. m/z found, 461.3 [M+H]⁺. ¹H NMR (400 MHz, DMSO-d₆) δ 9.02 (s, 1H), 8.42-8.36 (m, 1H), 8.19 (s, 1H), 8.07-8.00 (m, 1H), 7.49 (d, J=2.1 Hz, 1H), 7.37 (s, 1H), 7.08 (dd, J=7.3, 2.0 Hz, 1H), 6.37-6.30 (m, 1H), 6.08 (d, J=2.2 Hz, 1H), 4.71-4.59 (m, 1H), 3.79 (dd, J=10.7, 2.5 Hz, 2H), 3.74 (s, 3H), 3.50 (d, J=10.7 Hz, 2H), 2.66-2.58 (m, 2H), 2.45 (s, 3H), 2.44-2.35 (m, 5H), 1.69-1.57 (m, 2H).

### Example 424: 5-[2-methyl-5-[[(2S)-morpholin-2-yl]methoxy]-4-pyridyl]-N-[5-(trifluoromethyl)-2-pyridyl]pyrazolo[1,5-a]pyridin-2-amine

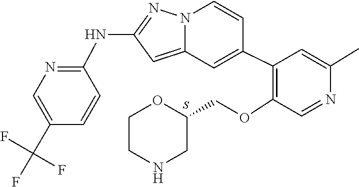

The title compound (77.6 mg, 14.4%). was prepared by the similar method of Example 116 Step A and Step B. In step A, 2-bromo-5-(trifluoromethyl)pyridine was used instead of 4-bromo-1-(difluoromethyl)-1H-pyrazole and (S)-tert-butyl 2-(((4-(2-aminopyrazolo[1,5-a]pyridin-5-yl)-6-methylpyridin-3-yl)oxy)methyl)morpholine-4-carboxylate (Intermediate 13) instead of tert-butyl (1R,5S,7s)-7-((4-(2-aminopyrazolo[1,5-a]pyridin-5-yl)-6-methylpyridin-3-yl)oxy)-3-oxa-9-azabicyclo[3.3.1]nonane-9-carboxylate (Intermediate 9) to afford the title compound. MS (ESI): mass calcd. for C₂₄H₂₃F₃N₆O₂, 484.2. m/z found, 485.1 [M+H]⁺. ¹H NMR (400 MHz, CD₃OD) δ 8.48 (s, 1H), 8.41 (d, J=7.2 Hz, 1H), 8.21 (s, 1H), 7.83 (dd, J=2.4, 8.8 Hz, 1H), 7.80 (s, 1H), 7.42 (d, J=9.2 Hz, 1H), 7.36 (s, 1H), 7.03 (dd, J=1.6, 7.2 Hz, 1H), 6.86 (s, 1H), 4.13 (d, J=4.8 Hz, 2H), 3.96-3.87 (m, 1H), 3.86-3.78 (m, 1H), 3.71-3.57 (m, 1H), 2.96-2.87 (m, 1H), 2.83-2.77 (m, 2H), 2.75-2.66 (m, 1H), 2.52 (s, 3H) ¹⁹F NMR (376 MHz, CD₃OD) δ −62.90 (s, 3F)

### Example 425: 5-[2-methyl-5-[[(2S)-morpholin-2-yl]methoxy]-4-pyridyl]-N-(5-methyl-2-pyridyl)pyrazolo[1,5-a]pyridin-2-amine

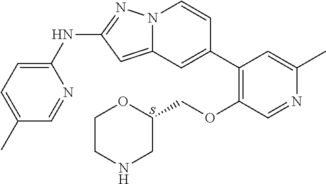

The title compound (29 mg, 8%) was prepared by the similar method of Example 116 Step A and Step B. In step A, 2-bromo-5-methylpyridine was used instead of 4-bromo-1-(difluoromethyl)-1H-pyrazole and (S)-tert-butyl 2-(((4-(2-aminopyrazolo[1,5-a]pyridin-5-yl)-6-methylpyridin-3-yl)oxy)methyl)morpholine-4-carboxylate (Intermediate 13) instead of tert-butyl (1R,5S,7s)-7-((4-(2-aminopyrazolo[1,5-a]pyridin-5-yl)-6-methylpyridin-3-yl)oxy)-3-oxa-9-azabicyclo[3.3.1]nonane-9-carboxylate (Intermediate 9) to afford the title compound. MS (ESI): mass calcd. for C₂₄H₂₆N₆O₂, 430.2. m/z found, 431.3 [M+H]⁺. ¹H NMR (400 MHz, CD₃CN) δ 8.34 (d, J=7.2 Hz, 1H), 8.25 (s, 1H), 8.07 (d, J=1.6 Hz, 1H), 7.95 (s, 1H), 7.76-7.66 (m, 1H), 7.47 (dd, J=2.4, 8.4 Hz, 1H), 7.30 (d, J=8.4 Hz, 1H), 7.26 (s, 1H), 6.96 (dd, J=2.0, 7.2 Hz, 1H), 6.72 (s, 1H), 4.12-3.98 (m, 2H), 3.83-3.75 (m, 1H), 3.74-3.66 (m, 1H), 3.57-3.43 (m, 1H), 2.85 (dd, J=2.4, 12.0 Hz, 1H), 2.73-2.66 (m, 2H), 2.59 (dd, J=10.4, 12.0 Hz, 1H), 2.47 (s, 3H), 2.23 (s, 3H).

### Example 426: 5-[2-methyl-5-[[(2S)-morpholin-2-yl]methoxy]-4-pyridyl]-N-(6-methyl-2-pyridyl)pyrazolo[1,5-a]pyridin-2-amine

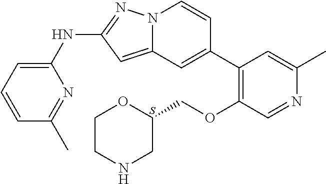

The title compound (46 mg, 15%) was prepared by the similar method of Example 116 Step A and Step B. In step A, 2-bromo-6-methylpyridine was used instead of 4-bromo-1-(difluoromethyl)-1H-pyrazole and (S)-tert-butyl 2-(((4-(2-aminopyrazolo[1,5-a]pyridin-5-yl)-6-methylpyridin-3-yl)oxy)methyl)morpholine-4-carboxylate (Intermediate 13) instead of tert-butyl (1R,5S,7s)-7-((4-(2-aminopyrazolo[1,5-a]pyridin-5-yl)-6-methylpyridin-3-yl)oxy)-3-oxa-9-azabicyclo[3.3.1]nonane-9-carboxylate (Intermediate 9) to afford the title compound. MS (ESI): mass calcd. for C₂₄H₂₆N₆O₂, 430.2. m/z found, 431.0 [M+H]⁺. ¹H NMR (400 MHz, CD₃CN) δ 8.35 (d, J=7.2 Hz, 1H), 8.26 (s, 1H), 7.89 (s, 1H), 7.77 (d, J=1.2 Hz, 1H), 7.58-7.43 (m, 1H), 7.27 (s, 1H), 7.14 (d, J=8.0 Hz, 1H), 6.97 (dd, J=2.0, 7.2 Hz, 1H), 6.86 (s, 1H), 6.70 (d, J=7.2 Hz, 1H), 4.12-3.98 (m, 2H), 3.85-3.76 (m, 1H), 3.75-3.66 (m, 1H), 3.59-3.47 (m, 1H), 2.85 (dd, J=2.4, 12.0 Hz, 1H), 2.74-2.67 (m, 2H), 2.60 (dd, J=10.4, 12.0 Hz, 1H), 2.48 (s, 3H), 2.43 (s, 3H).

### Example 427: 5-[2-methyl-5-[[(2S)-morpholin-2-yl]methoxy]-4-pyridyl]-N-(4-methyl-2-pyridyl)pyrazolo[1,5-a]pyridin-2-amine

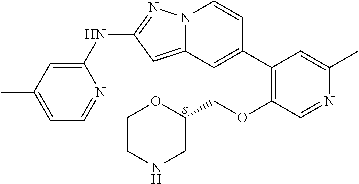

The title compound (58 mg, 24%) was prepared by the similar method of Example 116 Step A and Step B. In step A, 2-bromo-4-methylpyridine was used instead of 4-bromo-1-(difluoromethyl)-1H-pyrazole and (S)-tert-butyl 2-(((4-(2-aminopyrazolo[1,5-a]pyridin-5-yl)-6-methylpyridin-3-yl)oxy)methyl)morpholine-4-carboxylate (Intermediate 13) instead of tert-butyl (1R,5S,7s)-7-((4-(2-aminopyrazolo[1,5-a]pyridin-5-yl)-6-methylpyridin-3-yl)oxy)-3-oxa-9-azabicyclo[3.3.1]nonane-9-carboxylate (Intermediate 9) to afford the title compound. MS (ESI): mass calcd. for C₂₄H₂₆N₆O₂, 430.2. m/z found, 431.3 [M+H]⁺. ¹H NMR (400 MHz, CD₃CN) δ 8.35 (d, J=7.2 Hz, 1H), 8.26 (s, 1H), 8.09 (d, J=5.2 Hz, 1H), 7.93 (s, 1H), 7.73 (d, J=1.2 Hz, 1H), 7.26 (s, 1H), 7.20 (s, 1H), 6.98 (dd, J=2.0, 7.2 Hz, 1H), 6.77 (s, 1H), 6.69 (d, J=4.8 Hz, 1H), 4.11-4.00 (m, 2H), 3.79 (td, J=2.4, 11.2 Hz, 1H), 3.75-3.67 (m, 1H), 3.56-3.45 (m, 1H), 2.85 (dd, J=2.4, 12.4 Hz, 1H), 2.73-2.67 (m, 2H), 2.59 (dd, J=10.4, 12.4 Hz, 1H), 2.47 (s, 3H), 2.31 (s, 3H).

### Example 428: 5-[2-methyl-5-[[(2R)-morpholin-2-yl]methoxy]-4-pyridyl]-N-(2-pyridyl)pyrazolo[1,5-a]pyridin-2-amine

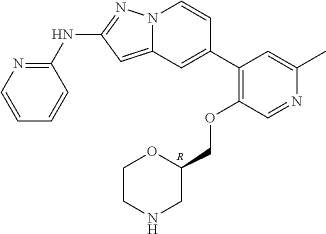

The title compound (90 mg, 38%) was prepared by the similar method of Example 116 Step A and Step B. In step A, 2-bromopyridine was used instead of 4-bromo-1-(difluoromethyl)-1H-pyrazole and (S)-tert-butyl 2-4(4-(2-aminopyrazolo[1,5-a]pyridin-5-yl)-6-methylpyridin-3-yl)oxy)methyl)morpholine-4-carboxylate (Intermediate 13) instead of tert-butyl (1R,5S,7s)-7-((4-(2-aminopyrazolo[1,5-a]pyridin-5-yl)-6-methylpyridin-3-yl)oxy)-3-oxa-9-azabicyclo[3.3.1]nonane-9-carboxylate (Intermediate 9) to afford the title compound. MS (ESI): mass calcd. for C₂₃H₂₄N₆O₂, 416.2. m/z found, 417.2 [M+H]⁺. ¹H NMR (400 MHz, DMSO-d₆) δ 9.77 (s, 1H), 8.54 (dt, J=7.2, 1.0 Hz, 1H), 8.32 (s, 1H), 8.21 (ddd, J=5.0, 2.0, 0.8 Hz, 1H), 7.82 (dd, J=2.0, 0.9 Hz, 1H), 7.62 (ddd, J=9.0, 7.1, 2.0 Hz, 1H), 7.36-7.30 (m, 2H), 7.00 (dd, J=7.2, 2.0 Hz, 1H), 6.85 (d, J=0.8 Hz, 1H), 6.80 (ddd, J=7.2, 4.9, 1.0 Hz, 1H), 4.14-4.05 (m, 2H), 3.77-3.72 (m, 1H), 3.67 (dtd, J=10.0, 4.9, 2.4 Hz, 1H), 3.44 (td, J=10.6, 3.6 Hz, 1H), 2.81 (dd, J=12.1, 2.4 Hz, 1H), 2.68-2.59 (m, 2H), 2.46 (s, 3H).

### Example 429: 5-[2-methyl-5-[[(2R)-morpholin-2-yl]methoxy]-4-pyridyl]-N-(1-methylpyrazol-3-yl)pyrazolo[1,5-a]pyridin-2-amine

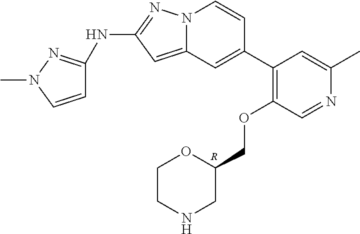

The title compound (92 mg, 80%) was prepared by the similar method of Example 116 Step A and Step B. In step A, 3-bromo-1-methyl-1H-pyrazole was used instead of 4-bromo-1-(difluoromethyl)-1H-pyrazole and (S)-tert-butyl 2-(((4-(2-aminopyrazolo[1,5-a]pyridin-5-yl)-6-methylpyridin-3-yl)oxy)methyl)morpholine-4-carboxylate (Intermediate 13) instead of tert-butyl (1R,5S,7s)-7-((4-(2-aminopyrazolo[1,5-a]pyridin-5-yl)-6-methylpyridin-3-yl)oxy)-3-oxa-9-azabicyclo[3.3.1]nonane-9-carboxylate (Intermediate 9) to afford the title compound. MS (ESI): mass calcd. for C₂₂H₂₅N₇O₂, 419.2. m/z found, 420.2 [M+H]⁺. ¹H NMR (400 MHz, DMSO-d₆) δ 9.05 (s, 1H), 8.44 (dd, J=7.2, 0.9 Hz, 1H), 8.30 (s, 1H), 7.73 (dd, J=2.1, 0.9 Hz, 1H), 7.49 (d, J=2.2 Hz, 1H), 7.33 (s, 1H), 6.91 (dd, J=7.2, 2.0 Hz, 1H), 6.41 (d, J=0.8 Hz, 1H), 6.06 (d, J=2.2 Hz, 1H), 4.12-4.03 (m, 2H), 3.73 (s, 3H), 3.67 (dtd, J=10.0, 4.8, 2.4 Hz, 1H), 3.44 (td, J=10.6, 3.7 Hz, 1H), 2.80 (dd, J=12.1, 2.4 Hz, 1H), 2.69-2.60 (m, 2H), 2.45 (s, 3H).

### Example 430: 5-[2-methyl-5-[[(2R)-morpholin-2-yl]methoxy]-4-pyridyl]-N-pyrazin-2-yl-pyrazolo[1,5-a]pyridin-2-amine

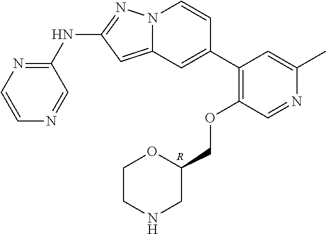

The title compound (80 mg, 4%) was prepared by the similar method of Example 116 Step A and Step B. In step A, 2-chloropyrazine was used instead of 4-bromo-1-(difluoromethyl)-1H-pyrazole and (S)-tert-butyl 2-4(4-(2-aminopyrazolo[1,5-a]pyridin-5-yl)-6-methylpyridin-3-yl)oxy)methyl)morpholine-4-carboxylate (Intermediate 13) instead of tert-butyl (1R,5S,7s)-7-((4-(2-aminopyrazolo[1,5-a]pyridin-5-yl)-6-methylpyridin-3-yl)oxy)-3-oxa-9-azabicyclo[3.3.1]nonane-9-carboxylate (Intermediate 9) to afford the title compound. MS (ESI): mass calcd. for C₂₂H₂₃N₇O₂, 417.2. m/z found, 418.2 [M+H]⁺. ¹H NMR (400 MHz, DMSO-d₆) δ 10.26 (s, 1H), 8.65 (s, 1H), 8.59 (d, J=7.3 Hz, 1H), 8.33 (s, 1H), 8.22 (d, J=2.3 Hz, 1H), 8.01 (d, J=2.8 Hz, 1H), 7.86 (d, J=1.9 Hz, 1H), 7.35 (s, 1H), 7.05 (dd, J=7.4, 2.0 Hz, 1H), 6.90 (s, 1H), 4.10 (d, J=4.3 Hz, 2H), 3.76 (d, J=11.1 Hz, 1H), 3.72-3.64 (m, 1H), 3.51-3.40 (m, 1H), 2.82 (d, J=11.8 Hz, 1H), 2.70-2.59 (m, 2H), 2.46 (s, 3H).

### Example 431: 5-[2-methyl-5-[[(2S)-morpholin-2-yl]methoxy]-4-pyridyl]-N-pyrazin-2-yl-pyrazolo[1,5-a]pyridin-2-amine

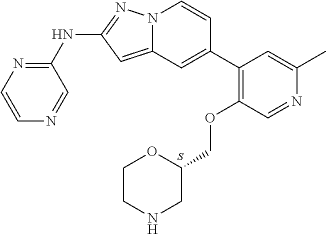

The title compound (82 mg, 62%) was prepared by the similar method of Example 430 except using the (S) enantiomer. MS (ESI): mass calcd. for C₂₂H₂₃N₇O₂, 417.2. m/z found, 418.2 [M+H]⁺. ¹H NMR (500 MHz, DMSO-d₆) δ 10.26 (s, 1H), 8.65 (d, J=1.4 Hz, 1H), 8.59 (dd, J=7.2, 1.0 Hz, 1H), 8.33 (s, 1H), 8.22 (dd, J=2.8, 1.4 Hz, 1H), 8.01 (dd, J=2.7, 1.1 Hz, 1H), 7.87 (d, J=2.0 Hz, 1H), 7.36 (s, 1H), 7.08-6.99 (m, 1H), 6.90 (d, J=1.0 Hz, 1H), 4.10 (dd, J=5.1, 2.8 Hz, 2H), 3.79-3.73 (m, 1H), 3.68 (d, J=9.1 Hz, 1H), 3.49-3.41 (m, 1H), 2.82 (d, J=12.2 Hz, 1H), 2.71-2.60 (m, 2H), 2.47 (s, 3H).

### Example 432: N-(2,6-dimethylpyrimidin-4-yl)-5-[2-methyl-5-[[(2S)-morpholin-2-yl]methoxy]-4-pyridyl]pyrazolo[1,5-a]pyridin-2-amine

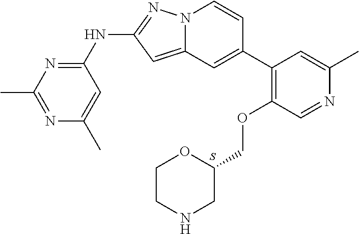

The title compound (120 mg, 48%) was prepared by the similar method of Example 136 using (S)-morpholin-2-ylmethanol instead of endo-7-hydroxy-3-oxa-9-azabicyclo[3.3.1]nonane hydrochloride and N-(2,6-dimethylpyrimidin-4-yl)-5-(5-fluoro-2-methylpyridin-4-yl)pyrazolo[1,5-a]pyridin-2-amine (Intermediate 15) instead of N-(5-(5-fluoro-2-methylpyridin-4-yl)pyrazolo[1,5-a]pyridin-2-yl)cyclopropanecarboxamide (Intermediate 6). MS (ESI): mass calcd. for C₂₄H₂₇N₇O₂, 445.2. m/z found, 446.2 [M+H]⁺. ¹H NMR (500 MHz, DMSO-d₆) δ 10.19 (s, 1H), 8.59 (d, J=7.2 Hz, 1H), 8.32 (s, 1H), 7.92 (d, J=1.1 Hz, 1H), 7.37 (s, 1H), 7.07 (dd, J=7.2, 2.0 Hz, 1H), 7.00 (br s, 1H), 6.88 (br s, 1H), 4.10 (d, J=4.8 Hz, 2H), 3.80-3.74 (m, 1H), 3.71-3.64 (m, 1H), 3.46 (td, J=10.5, 3.8 Hz, 1H), 2.80 (dd, J=12.0, 2.4 Hz, 1H), 2.70-2.60 (m, 2H), 2.56-2.51 (m, 1H), 2.46 (s, 3H), 2.46 (s, 3H), 2.30 (s, 3H).

### Example 433: N-[5-[5-[(3-aminooxetan-3-yl)methoxy]-2-prop-1-ynyl-4-pyridyl]pyrazolo[1,5-a]pyridin-2-yl]cyclopropanecarboxamide

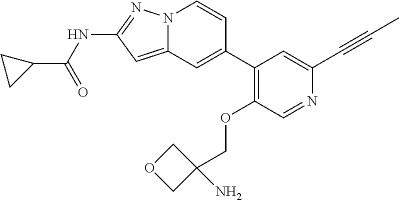 [SMILES: CC#Cc1cc(-c2ccn3nc(NC(=O)C4CC4)cc3c2)c(OCC2(N)COC2)cn1]

The title compound (22 mg, 15%) was prepared by the similar method of Example 136 using (3-aminooxetan-3-yl)methanol instead of endo-7-hydroxy-3-oxa-9-azabicyclo[3.3.1]nonane hydrochloride and N-(5-(5-fluoro-2-(prop-1-yn-1-yl)pyridin-4-yl)pyrazolo[1,5-a]pyridin-2-yl)cyclopropanecarboxamide (Intermediate 11) instead of N-(5-(5-fluoro-2-methylpyridin-4-yl)pyrazolo[1,5-a]pyridin-2-yl)cyclopropanecarboxamide (Intermediate 6). MS (ESI): mass calcd. for C₂₃H₂₃N₅O₃, 417.2. m/z found, 418.2 [M+H]⁺. ¹H NMR (400 MHz, DMSO-d₆) δ 11.09 (s, 1H), 8.53 (d, J=7.3 Hz, 1H), 8.48 (s, 1H), 7.96 (dd, J=2.0, 0.9 Hz, 1H), 7.56 (s, 1H), 7.11 (dd, J=7.3, 2.0 Hz, 1H), 6.87 (s, 1H), 4.45 (d, J=6.1 Hz, 2H), 4.32 (d, J=6.0 Hz, 2H), 4.27 (s, 2H), 2.22 (s, 2H), 2.07 (s, 3H), 1.98-1.87 (m, 1H), 0.89-0.75 (m, 4H).

### Example 434: N-[5-[5-[(3-fluoroazetidin-3-yl)methoxy]-2-prop-1-ynyl-4-pyridyl]pyrazolo[1,5-a]pyridin-2-yl]cyclopropanecarboxamide

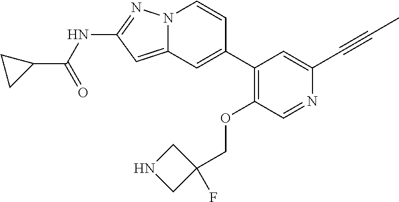 [SMILES: CC#Cc1cc(-c2ccn3nc(NC(=O)C4CC4)cc3c2)c(OCC2(F)CNC2)cn1]

The title compound (41 mg, 33%) was prepared by the similar method of Example 136 using (3-fluoroazetidin-3-yl)methanol instead of endo-7-hydroxy-3-oxa-9-azabicyclo[3.3.1]nonane hydrochloride and N-(5-(5-fluoro-2-(prop-1-yn-1-yl)pyridin-4-yl)pyrazolo[1,5-a]pyridin-2-yl)cyclopropanecarboxamide (Intermediate 11) instead of N-(5-(5-fluoro-2-methylpyridin-4-yl)pyrazolo[1,5-a]pyridin-2-yl)cyclopropanecarboxamide (Intermediate 6). MS (ESI): mass calcd. for C₂₃H₂₂FN₅O₂, 419.2. m/z found, 420.2 [M+H]⁺. ¹H NMR (400 MHz, DMSO-d₆) δ 11.10 (s, 1H), 8.60-8.52 (m, 1H), 8.49 (s, 1H), 7.85 (dd, J=2.0, 0.9 Hz, 1H), 7.56 (s, 1H), 7.04 (dd, J=7.3, 2.0 Hz, 1H), 6.87 (s, 1H), 4.56 (d, J=23.7 Hz, 2H), 3.59 (dd, J=20.1, 9.7 Hz, 2H), 3.53-3.44 (m, 2H), 2.07 (s, 3H), 1.93 (qd, J=7.7, 4.9 Hz, 1H), 0.82 (tt, J=7.7, 3.0 Hz, 4H).

### Example 435: N-(5-(5-((1r,3r)-3-aminocyclobutoxy)-2-(prop-1-yn-1-yl)pyridin-4-yl)pyrazolo[1,5-a]pyridin-2-yl)cyclopropanecarboxamide

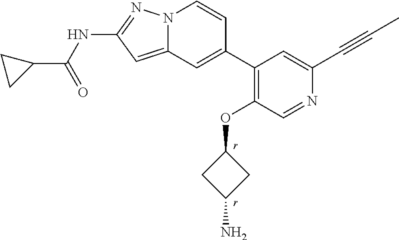 [SMILES: CC#Cc1cc(-c2ccn3nc(NC(=O)C4CC4)cc3c2)c(O[C@H]2C[C@H](N)C2)cn1]

The title compound (34 mg, 19%) was prepared by the similar method of Example 136 using trans-3-aminocyclobutanoyl instead of endo-7-hydroxy-3-oxa-9-azabicyclo[3.3.1]nonane hydrochloride and N-(5-(5-fluoro-2-(prop-1-yn-1-yl)pyridin-4-yl)pyrazolo[1,5-a]pyridin-2-yl)cyclopropanecarboxamide (Intermediate 11) instead of N-(5-(5-fluoro-2-methylpyridin-4-yl)pyrazolo[1,5-a]pyridin-2-yl)cyclopropanecarboxamide (Intermediate 6). MS (ESI): mass calcd. for C₂₃H₂₃N₅O₂, 401.2. m/z found, 402.2 [M+H]⁺. ¹H NMR (400 MHz, DMSO-d₆) δ11.09 (s, 1H), 8.56 (d, J=7.2 Hz, 1H), 8.13 (s, 1H), 7.85 (d, J=1.9 Hz, 1H), 7.53 (s, 1H), 7.07 (dd, J=7.2, 2.0 Hz, 1H), 6.90 (s, 1H), 5.05 (tt, J=6.9, 4.1 Hz, 1H), 3.55 (tt, J=7.6, 5.2 Hz, 1H), 2.28 (ddd, J=11.9, 7.6, 3.8 Hz, 2H), 2.20-2.10 (m, 2H), 2.06 (s, 3H), 1.92 (td, J=7.6, 3.9 Hz, 1H), 0.90-0.77 (m, 4H).

### Example 436: N-[5-[5-[[(1S,5R)-3-oxa-9-azabicyclo[3.3.1]nonan-7-yl]oxy]-2-prop-1-ynyl-4-pyridyl]pyrazolo[1,5-a]pyridin-2-yl]cyclopropanecarboxamide

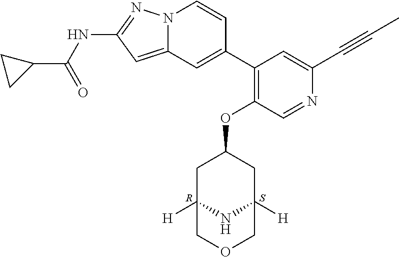

The title compound (41 mg, 33%) was prepared by the similar method of Example 136 except N-(5-(5-fluoro-2-(prop-1-yn-1-yl)pyridin-4-yl)pyrazolo[1,5-a]pyridin-2-yl)cyclopropanecarboxamide (Intermediate 11) instead of N-(5-(5-fluoro-2-methylpyridin-4-yl)pyrazolo[1,5-a]pyridin-2-yl)cyclopropanecarboxamide (Intermediate 6). MS (ESI): mass calcd. for C₂₆H₂₇N₅O₃, 457.2. m/z found, 458.2 [M+H]⁺. ¹H NMR (400 MHz, DMSO-d₆) δ 11.08 (s, 1H), 8.52 (d, J=7.3 Hz, 1H), 8.36 (s, 1H), 8.13 (dd, J=2.0, 0.9 Hz, 1H), 7.55 (s, 1H), 7.19 (dd, J=7.3, 2.1 Hz, 1H), 6.85 (s, 1H), 4.80 (t, J=5.9 Hz, 1H), 3.63-3.48 (m, 4H), 2.96-2.88 (m, 2H), 2.36-2.25 (m, 2H), 2.06 (s, 2H), 1.92 (s, 1H), 1.70 (dd, J=13.5, 5.1 Hz, 1H), 0.88-0.76 (m, 4H).

### Example 437: 5-[5-[(3-fluoroazetidin-3-yl)methoxy]-2-prop-1-ynyl-4-pyridyl]-N-(1-methylpyrazol-3-yl)pyrazolo[1,5-a]pyridin-2-amine

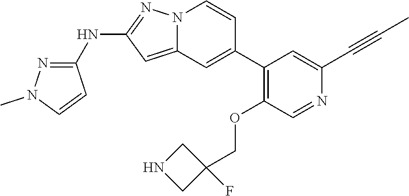 [SMILES: CC#Cc1cc(-c2ccn3nc(Nc4ccn(C)n4)cc3c2)c(OCC2(F)CNC2)cn1]

The title compound (15.3 mg, 12%) was prepared by the similar method of Example 136 using (3-fluoroazetidin-3-yl)methanol instead of endo-7-hydroxy-3-oxa-9-azabicyclo[3.3.1]nonane hydrochloride and N-(5-(5-fluoro-2-(prop-1-yn-1-yl)pyridin-4-yl)pyrazolo[1,5-a]pyridin-2-yl)cyclopropanecarboxamide (Intermediate 19) instead of N-(5-(5-fluoro-2-methylpyridin-4-yl)pyrazolo[1,5-a]pyridin-2-yl)cyclopropanecarboxamide (Intermediate 6). MS (ESI): mass calcd. for C₂₃H₂₂FN₇O, 431.2. m/z found, 432.2 [M+H]⁺. ¹H NMR (400 MHz, DMSO-d₆) δ 9.07 (s, 1H), 8.48 (s, 1H), 8.44 (dt, J=7.2, 0.9 Hz, 1H), 7.69 (dd, J=2.0, 0.9 Hz, 1H), 7.55-7.44 (m, 2H), 6.86 (dd, J=7.1, 2.0 Hz, 1H), 6.40 (d, J=0.9 Hz, 1H), 6.05 (d, J=2.2 Hz, 1H), 4.55 (d, J=23.7 Hz, 2H), 3.73 (s, 3H), 3.65-3.42 (m, 4H), 2.07 (s, 3H).

### Example 438: N-(1-methylpyrazol-3-yl)-5-[5-[[(1S,5R)-3-oxa-9-azabicyclo[3.3.1]nonan-7-yl]oxy]-2-prop-1-ynyl-4-pyridyl]pyrazolo[1,5-a]pyridin-2-amine

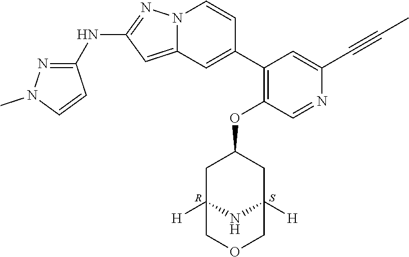

The title compound (15.3 mg, 12%) was prepared by the similar method of Example 136 using N-(5-(5-fluoro-2-(prop-1-yn-1-yl)pyridin-4-yl)pyrazolo[1,5-a]pyridin-2 yl)cyclopropanecarboxamide (Intermediate 19) instead of N-(5-(5-fluoro-2-methylpyridin-4-yl)pyrazolo[1,5-a]pyridin-2-yl)cyclopropanecarboxamide (Intermediate 6). MS (ESI): mass calcd. for C₂₆H₂₇N₇O₂, 469.2. m/z found, 470.3 [M+H]⁺. ¹H NMR (400 MHz, DMSO-d₆) δ 9.04 (s, 1H), 8.42-8.37 (m, 1H), 8.36 (s, 1H), 7.91 (dd, J=2.0, 0.9 Hz, 1H), 7.51 (s, 1H), 7.48 (d, J==2.2 Hz, 1H), 7.00 (dd, J=7.2, 2.0 Hz, 1H), 6.38 (d, J=0.8 Hz, 1H), 6.05 (d, J=2.2 Hz, 1H), 4.77 (q, J=6.3 Hz, 1H), 3.73 (s, 3H), 3.61-3.47 (m, 4H), 2.94 (d, J=8.2 Hz, 2H), 2.31 (dt, J=14.3, 7.5 Hz, 3H), 2.06 (s, 3H), 1.68 (dd, J=11.2, 4.7 Hz, 2H).

### Example 439: N-[5-[5-[[(1S,5R)-3-oxa-9-azabicyclo[3.3.1]nonan-7-yl]oxy]-2-prop-1-ynyl-4-pyridyl]pyrazolo[1,5-a]pyridin-2-yl]cyclopropanecarboxamide

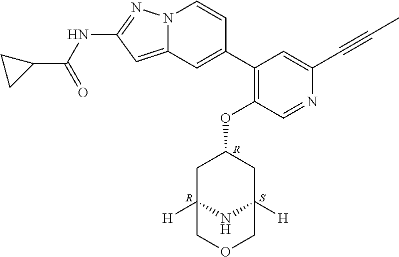

The title compound (14 mg, 11%) was prepared by the similar method of Example 8 using endo-9-boc-7-hydroxy-3-oxa-9-azabicyclo[3.3.1]nonane was used instead of (2S,4R)-tert-butyl 4-hydroxy-2-(trifluoromethyl)piperidine-1-carboxylate and N-(5-(5-fluoro-2-(prop-1-yn-1-yl)pyridin-4-yl)pyrazolo[1,5-a]pyridin-2-yl)cyclopropanecarboxamide (Intermediate 11) instead of N-(5-(2-cyano-5-fluoropyridin-4-yl)pyrazolo[1,5-a]pyridin-2-yl)cyclopropanecarboxamide to afford the title compound. MS (ESI): mass calcd. for C₂₆H₂₇N₅O₃, 457.2. m/z found, 458.2 [M+H]⁺. ¹H NMR (500 MHz, DMSO-d₆) δ 11.08 (s, 1H), 8.55 (dt, J=7.3, 0.9 Hz, 1H), 8.40 (s, 1H), 7.85 (dd, J=2.0, 0.9 Hz, 1H), 7.52 (s, 1H), 7.06 (dd, J=7.2, 2.0 Hz, 1H), 6.89 (s, 1H), 5.45 (tt, J=11.0, 6.0 Hz, 1H), 3.75-3.68 (m, 2H), 3.62 (dt, J=10.9, 2.3 Hz, 2H), 2.93 (s, 2H), 2.30-2.21 (m, 2H), 2.06 (s, 3H), 1.93 (p, J=7.5 Hz, 1H), 1.70 (ddd, J=16.3, 8.3, 4.6 Hz, 2H), 0.82 (tt, J=7.9, 3.0 Hz, 4H).

### Example 440: N-(2,6-dimethylpyrimidin-4-yl)-5-[5-[[(1S,4S,7R)-2-oxa-5-azabicyclo[2.2.1]heptan-7-yl]oxy]-2-prop-1-ynyl-4-pyridyl]pyrazolo[1,5-a]pyridin-2-amine

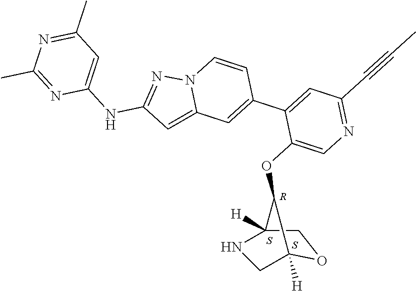

Step A. tert-butyl (1S,4S,7R)-7-((6-chloro-4-(2-((2,6-dimethylpyrimidin-4-yl)amino)pyrazolo[1,5-a]pyridin-5-yl)pyridin-3-yl)oxy)-2-oxa-5-azabicyclo[2.2.1]heptane-5-carboxylate. To a 20 mL vial charged with tert-butyl (1S,4S,7R)-7-hydroxy-2-oxa-5-azabicyclo[2.2.1]heptane-5-carboxylate (115 mg, 0.534 mmol) and 2.1 mL DMA was added sodium tert-butoxide (256.7 mg, 2.67 mmol). The mixture was stirred at 50° C. for 5 min. Then, added 5-(2-chloro-5-fluoropyridin-4-yl)-N-(2,6-dimethylpyrimidin-4-yl)pyrazolo[1,5-a]pyridin-2-amine (197 mg, 0.534 mmol), then stirred at 50° C. for 20 min. The crude was partitioned between EtOAc/water/brine. The organic layer was collected, and dried over anh. Na₂SO₄, filtered, and concentrated down under vacuo. The crude was FCC 20-100% EtOAc (with 10% MeOH)/hexanes, 20 min gradient to afford the titled compound (421 mg, 57.4%). MS (ESI): mass calcd. for C₂₈H₃₀ClN₇O₄, 563.2. m/z found, 564.2 [M+H]⁺.

Step B and Step C. 5-(5-(((1S,4S,7R)-2-oxa-5-azabicyclo[2.2.1]heptan-7-yl)oxy)-2-(prop-1-yn-1-yl)pyridin-4-yl)-N-(2,6-dimethylpyrimidin-4-yl)pyrazolo[1,5-a]pyridin-2-amine.

The title compound was prepared by the similar method of Step B in Example 441. In Step B, 5-(2-chloro-5-fluoropyridin-4-yl)-N-(2,6-dimethylpyrimidin-4-yl)pyrazolo[1,5-a]pyridin-2-amine was used instead of (R)—N-(5-(2-chloro-5-((1-methylpyrrolidin-3-yl)methoxy)pyridin-4-yl)pyrazolo[1,5-a]pyridin-2-yl)cyclopropanecarboxamide. The Boc-protecting group was also removed by adding 1.5 mL 1:10 TFA:DCM to the purified product from Step B. (20 mg, 2 steps, 21%). MS (ESI): mass calcd. for C₂₆H₂₅N₇O₂, 467.2. m/z found, 468.2 [M+H]⁺. ¹H NMR (600 MHz, DMSO-d₆) δ 10.20 (s, 1H), 8.61 (s, 1H), 8.60-8.57 (m, 1H), 7.83-7.78 (m, 1H), 7.54 (s, 1H), 7.06-6.96 (m, 2H), 6.84 (s, 1H), 4.97 (d, J=2.3 Hz, 1H), 4.31 (s, 1H), 3.88 (dd, J=7.3, 2.0 Hz, 1H), 3.74 (d, J=7.2 Hz, 1H), 3.48 (t, J=2.2 Hz, 1H), 3.14-3.05 (m, 1H), 2.80 (d, J=10.5 Hz, 1H), 2.46 (s, 3H), 2.30 (s, 3H), 2.07 (s, 3H).

### Example 441: N-[5-[5-[[(3R)-1-methylpyrrolidin-3-yl]methoxy]-2-prop-1-ynyl-4-pyridyl]pyrazolo[1,5-a]pyridin-2-yl]cyclopropanecarboxamide

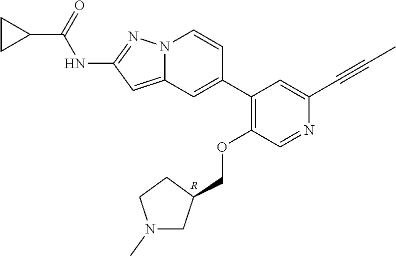

Step A. (R)—N-(5-(2-chloro-5-((1-methylpyrrolidin-3-yl)methoxy)pyridin-4-yl)pyrazolo[1,5-a]pyridin-2-yl)cyclopropanecarboxamide. To a 100 mL rb flask was charged with N-[5-(4,4,5,5-tetramethyl-1,3,2-dioxaborolan-2-yl)pyrazolo[1,5-a]pyridin-2-yl]cyclopropanecarboxamide (Intermediate 3, 1349 mg, 4.082 mmol), (R)-2-chloro-4-iodo-5-((1-methylpyrrolidin-3-yl)methoxy)pyridine (1427 mg, 4.047 mmol), and PdCl₂(dppf) (83 mg, 0.113 mmol) as solids. The flask was equipped with a septum then evac and backfilled with N₂ (2×). 1,4-dioxane (13.49 mL, 0.3 M, 4.047 mmol) and tribasic potassium phosphate 0.5 M in water (10.704 mL, 0.5 M, 5.352 mmol) were then added. The flask was placed into a preheated 60° C. heating block for 1 h. The reaction mixture was poured into 75 mL of stirring water. The resulting mixture was stirred for 30 min. Solids were collected by suction filtration and dried further using high vacuum at 65° C. for 2 h. MS (ESI): mass calcd. for C₂₂H₂₄ClN₅O₂, 425.2. m/z found, 426.2 [M+H]⁺.

Step B. (R)—N-(5-(5-((1-methylpyrrolidin-3-yl)methoxy)-2-(prop-1-yn-1-yl)pyridin-4-yl)pyrazolo[1,5-a]pyridin-2-yl)cyclopropanecarboxamide. To a 10 mL microwave vial was charged (R)—N-(5-(2-chloro-5-((1-methylpyrrolidin-3-yl)methoxy)pyridin-4-yl)pyrazolo[1,5-a]pyridin-2-yl)cyclopropanecarboxamide (Step A) (145 mg, 0.34 mmol) and Bis(triphenylphosphine)palladium(II) dichloride (20 mg, 0.0285 mmol) as solids. The flask was evacuated and backfilled with argon (3×). A portion of 1,4-dioxane (1.1 mL, 0.3 M, 0.33 mmol) was added followed by a solution of tributyl(1-propynyl)tin (147 mg, 0.447 mmol) in the remainder of dioxane. The vial was immediately placed into a preheated 70° C. heating block. The dark brown solution was cooled to r.t. then transferred to a 50 mL recovery vial by diluting with ethylacetate (15 mL). 1.5 g celite was added then conc to dryness for FCC (0-10% 2M NH₃-MeOH/DCM (15 column volumes) to afford (50 mg, 34.2%). MS (ESI): mass calcd. for C₂₅H₂₇N₅O₂, 429.2. m/z found, 430.3 [M+H]⁺. ¹H NMR (400 MHz, DMSO-d₆) δ 11.09 (s, 1H), 8.61-8.50 (m, 1H), 8.41 (s, 1H), 7.86 (d, J=1.9 Hz, 1H), 7.52 (s, 1H), 7.05 (dd, J=7.3, 2.0 Hz, 1H), 6.88 (s, 1H), 4.08 (d, J=6.1 Hz, 2H), 2.53 (dt, J=6.3, 3.1 Hz, 2H), 2.40 (ddt, J=9.3, 5.6, 2.8 Hz, 2H), 2.31 (dd, J=8.3, 4.8 Hz, 1H), 2.20 (s, 3H), 2.06 (s, 3H), 1.97-1.83 (m, 2H), 1.51 (dq, J=12.7, 6.8 Hz, 1H), 0.89-0.76 (m, 4H).

### Example 442: N-[5-[5-(1-methylazetidin-3-yl)oxy-2-prop-1-ynyl-4-pyridyl]pyrazolo[1,5-a]pyridin-2-yl]cyclopropanecarboxamide

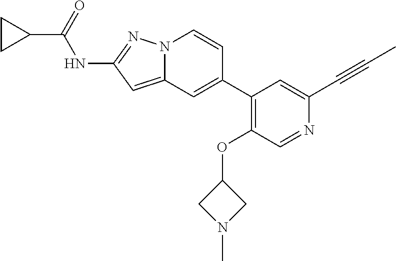 [SMILES: CC#Cc1cc(-c2ccn3nc(NC(=O)C4CC4)cc3c2)c(OC2CN(C)C2)cn1]

The title compound (72 mg, 47%) was prepared by the similar method of Example 441. In Step A, 2-chloro-4-iodo-5-((1-methylazetidin-3-yl)oxy)pyridine was used instead of (R)-2-chloro-4-iodo-5-((1-methylpyrrolidin-3-yl)methoxy)pyridine. MS (ESI): mass calcd. for C₂₃H₂₃N₅O₂, 401.2. m/z found, 402.3 [M+H]⁺. ¹H NMR (400 MHz, DMSO-d₆) δ 11.09 (s, 1H), 8.57 (dd, J=7.3, 1.0 Hz, 1H), 8.13 (s, 1H), 7.86 (dd, J=2.0, 0.9 Hz, 1H), 7.54 (s, 1H), 7.07 (dd, J=7.2, 2.0 Hz, 1H), 6.91 (s, 1H), 4.97 (p, J=5.4 Hz, 1H), 3.80-3.59 (m, 2H), 3.08-2.95 (m, 2H), 2.26 (s, 3H), 2.06 (s, 3H), 1.98-1.86 (m, 1H), 0.91-0.72 (m, 4H).

### Example 443: N-[5-[5-[[(2S)-morpholin-2-yl]methoxy]-2-prop-1-ynyl-4-pyridyl]pyrazolo[1,5-a]pyridin-2-yl]cyclopropanecarboxamide

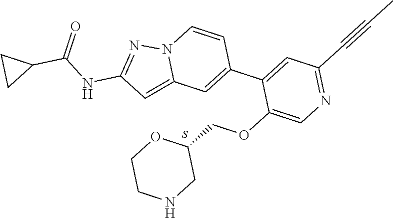

The title compound (75 mg, 52%) was prepared by the similar method of Example 441. In Step A, tert-butyl (S)-2-(((6-chloro-4-iodopyridin-3-yl)oxy)methyl)morpholine-4-carboxylate was used instead of (R)-2-chloro-4-iodo-5-((1-methylpyrrolidin-3-yl)methoxy)pyridine. The Boc-protecting group was removed by adding 1.5 mL 1:10 TFA:DCM to the purified product from Step B. MS (ESI): mass calcd. for C₂₄H₂₅N₅O₃, 431.2. m/z found, 432.2 [M+H]⁺. ¹H NMR (400 MHz, DMSO-d₆) δ 11.09 (s, 1H), 8.56 (dt, J=7.3, 0.9 Hz, 1H), 8.40 (s, 1H), 7.93 (dd, J=2.0, 0.9 Hz, 1H), 7.54 (s, 1H), 7.10 (dd, J=7.3, 2.0 Hz, 1H), 6.89 (s, 1H), 4.17 (d, J=4.8 Hz, 2H), 3.75 (d, J=11.0 Hz, 1H), 3.71-3.62 (m, 1H), 3.45 (td, J=10.8, 3.1 Hz, 1H), 2.87-2.75 (m, 1H), 2.70-2.56 (m, 2H), 2.44-2.28 (m, 1H), 2.06 (s, 3H), 1.93 (m, J=7.7, 3.8 Hz, 1H), 0.89-0.74 (m, 4H).

### Example 444: N-(5-(5-((1r,3r)-3-amino-3-methylcyclobutoxy)-2-methylpyridin-4-yl)pyrazolo[1,5-a]pyridin-2-yl)cyclopropanecarboxamide

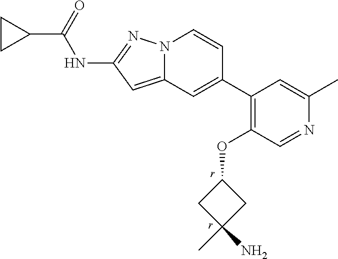 [SMILES: Cc1cc(-c2ccn3nc(NC(=O)C4CC4)cc3c2)c(O[C@H]2C[C@](C)(N)C2)cn1]

The title compound (222 mg, 88%) was prepared by the similar method of Example 1 using trans-3-amino-3-methylcyclobutanol hydrochloride instead of (R)-(1-methylpyrrolidin-3-yl)methanol. MS (ESI): mass calcd. for C₂₂H₂₅N₅O₂, 391.2. m/z found, 392.2 [M+H]⁺. ¹H NMR (500 MHz, CD₃Cl) δ 10.13 (s, 1H), 8.23 (d, J=7.2 Hz, 1H), 7.99 (s, 1H), 7.60 (d, J=1.9 Hz, 1H), 7.08 (s, 1H), 6.99 (s, 1H), 6.87 (dd, J=7.2, 2.0 Hz, 1H), 4.85-4.76 (m, 1H), 2.44 (s, 3H), 2.38-2.30 (m, 2H), 2.05 (dd, J=13.0, 5.2 Hz, 2H), 1.62-1.50 (m, 1H), 1.25 (s, 3H), 1.06 (p, J=4.2 Hz, 2H), 0.79-0.72 (m, 2H).

### Example 445: N-[5-[2-methyl-5-[[(3*S,5*R)-5-(trifluoromethyl)-3-piperidyl]oxy]-4-pyridyl]pyrazolo[1,5-a]pyridin-2-yl]cyclopropanecarboxamide

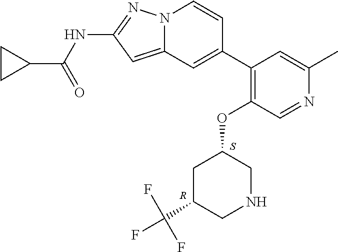

Step A. tert-butyl (2*S,4*R)-4-((4-(2-(cyclopropanecarboxamido)pyrazolo[1,5-a]pyridin-5-yl)-6-methylpyridin-3-yl)oxy)-2-(trifluoromethyl)piperidine-1-carboxylate. The protected version of the title compound was prepared by the similar method of Example 1 using (3*S,5*R)-5-(trifluoromethyl)piperidin-3-ol instead of (R)-(1-methylpyrrolidin-3-yl)methanol (222 mg, 88%).

Step B. N-[5-[2-methyl-5-[[(3*S,5*R)-5-(trifluoromethyl)-3-piperidyl]oxy]-4-pyridyl]pyrazolo[1,5-a]pyridin-2-yl]cyclopropanecarboxamide. The Boc-protecting group was removed by stirring the product from Step A with 1:10 TFA:DCM (11 mL) to give N-(5-(2-methyl-5-4(2*S,4*R)-2-(trifluoromethyl)piperidin-4-yl)oxy)pyridin-4-yl)pyrazolo[1,5-a]pyridin-2-yl)cyclopropanecarboxamide (70 mg, 0.152 mmol) which was further purified by SFC over DAICEL CHIRALCEL OD-H 250 mm×30 mm, 5 μm (eluent: 30% to 30% (v/v) 0.1% NH₃—H₂O MeOH). The pure fractions were collected, and the volatiles were removed under reduced pressure. The product was suspended in water (10 mL), the mixture frozen using dry ice/acetone, and then lyophilized to dryness to afford a white solid (8.30 mg, 11%). MS (ESI): mass calcd. for C₂₃H₂₄F₃N₅O₂, 459.2. m/z found, 460.2 [M+H]⁺. ¹H NMR (400 MHz, DMSO-d₆) δ 11.11 (s, 1H), 8.57 (d, J=7.2 Hz, 1H), 8.43 (s, 1H), 7.80 (d, J=1.2 Hz, 1H), 7.36 (s, 1H), 7.03 (dd, J=2.0, 7.2 Hz, 1H), 6.91 (s, 1H), 4.42-4.23 (m, 1H), 3.18 (dd, J=2.8, 11.6 Hz, 1H), 3.06-2.98 (m, 1H), 2.47 (s, 3H), 2.44-2.18 (m, 5H), 2.01-1.88 (m, 1H), 1.45-1.28 (m, 1H), 0.88-0.77 (m, 4H). ¹⁹F NMR (376 MHz, DMSO-d₆) δ −70.99 (d, J=9.0 Hz, 3F).

### Example 446: N-[5-[2-methyl-5-[[(3*R,5*S)-5-(trifluoromethyl)-3-piperidyl]oxy]-4-pyridyl]pyrazolo[1,5-a]pyridin-2-yl]cyclopropanecarboxamide

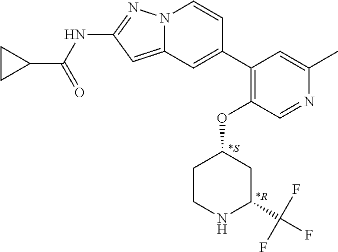

The title compounds was isolated by SFC resolution as the enantiomer of Example 445 using DAICEL CHIRALCEL OD-H 250 mm×30 mm, 5 μm (eluent: 30% to 30% (v/v) 0.1% NH₃—H₂O MEOH). The pure fractions were collected, and the volatiles were removed under reduced pressure. The product was suspended in water (10 mL), the mixture frozen using dry ice/acetone, and then lyophilized to dryness to afford a white solid (7.4 mg, 10.3%). MS (ESI): mass calcd. for C₂₃H₂₄F₃N₅O₂, 459.2. m/z found, 460.2 [M+H]⁺. ¹H NMR (400 MHz, DMSO-d₆) δ 11.10 (s, 1H), 8.57 (d, J=7.2 Hz, 1H), 8.42 (s, 1H), 7.81-7.79 (m, 1H), 7.36 (s, 1H), 7.04 (dd, J=1.6, 6.8 Hz, 1H), 6.90 (s, 1H), 4.61-4.44 (m, 1H), 3.04-2.94 (m, 1H), 2.60-2.54 (m, 1H), 2.48-2.43 (m, 4H), 2.24-2.11 (m, 1H), 2.02-1.87 (m, 2H), 1.43-1.26 (m, 2H), 0.89-0.77 (m, 4H) ¹⁹F NMR (376 MHz, DMSO-d₆) δ −75.54 (d, J=7.5 Hz, 3F)

### Example 447: (*R)—N-[5-[5-[(3-fluoropyrrolidin-3-yl)methoxy]-2-methyl-4-pyridyl]pyrazolo[1,5-a]pyridin-2-yl]cyclopropanecarboxamide

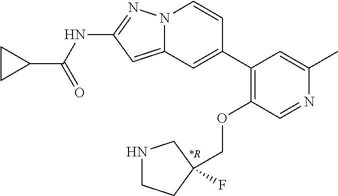

The title compound was prepared by the similar method of Example 445 using tert-butyl 3-fluoro-3-(hydroxymethyl)pyrrolidine-1-carboxylate instead of (R)-(1-methylpyrrolidin-3-yl)methanol. In Step A, the racemates were separated by SFC over DAICEL CHIRALCEL OJ-H (250 mm×30 mm, 5 um), (eluent: 35% to 35% (v/v) supercritical CO₂ in EtOH and H₂O with 0.1% NH₃) and the first eluting peak was collected (75 mg, 29.9%). Step B was similar to Example 445 as a white solid. (19.9 mg, 33%). MS (ESI): mass calcd. for C₂₂H₂₄FN₅O₂, 409.2; m/z found, 410.2 [M+H]⁺. ¹H NMR (400 MHz, CDCl₃) δ 8.51 (s, 1H), 8.28 (d, J=7.2 Hz, 1H), 8.25 (s, 1H), 7.68 (d, J=0.4 Hz, 1H), 7.20 (s, 1H), 7.02 (s, 1H), 6.97 (dd, J=1.6, 7.2 Hz, 1H), 4.37-4.18 (m, 2H), 3.28-3.16 (m, 2H), 2.98-2.84 (m, 2H), 2.57 (s, 3H), 2.18-1.86 (m, 2H), 1.65-1.53 (m, 1H), 1.19-1.11 (m, 2H), 0.96-0.88 (m, 2H). ¹⁹F NMR (376 MHz, CDCl₃) δ −149.81-150.63 (m, 1F)

### Example 448: (*S)—N-[5-[5-[(3-fluoropyrrolidin-3-yl)methoxy]-2-methyl-4-pyridyl]pyrazolo[1,5-a]pyridin-2-yl]cyclopropanecarboxamide

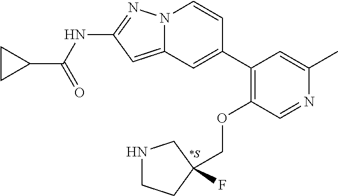

The title compound was prepared by the similar method of Example 445 using tert-butyl 3-fluoro-3-(hydroxymethyl)pyrrolidine-1-carboxylate instead of (R)-(1-methylpyrrolidin-3-yl)methanol. In Step A, the racemates were separated by SFC over DAICEL CHIRALCEL OJ-H (250 mm×30 mm, 5 um), (eluent: 35% to 35% (v/v) supercritical CO₂ in EtOH and H₂O with 0.1% NH₃) and the second eluting peak was collected (75 mg, 29.6%). Step B was similar to Example 445 as a white solid (22 mg, 36%). MS (ESI): mass calcd. for C₂₂H₂₄FN₅O₂, 409.2. m/z found, 410.2 [M+H]⁺. ¹H NMR (400 MHz, CDCl₃) δ 8.46 (s, 1H), 8.28 (d, J=7.2 Hz, 1H), 8.25 (s, 1H), 7.68 (d, J=0.8 Hz, 1H), 7.20 (s, 1H), 7.01 (s, 1H), 6.97 (dd, J=1.6, 7.2 Hz, 1H), 4.38-4.16 (m, 2H), 3.30-3.13 (m, 2H), 3.00-2.82 (m, 2H), 2.57 (s, 3H), 2.19-1.84 (m, 2H), 1.65-1.51 (m, 1H), 1.19-1.09 (m, 2H), 0.97-0.86 (m, 2H) ¹⁹F NMR (376 MHz, CDCl₃) δ −150.10-−150.16 (m, 1F)

### Example 449: *R—N-[5-[5-[(3-fluoro-1-methyl-pyrrolidin-3-yl)methoxy]-2-methyl-4-pyridyl]pyrazolo[1,5-a]pyridin-2-yl]cyclopropanecarboxamide

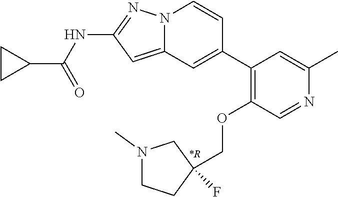

A solution of (*R)—N-[5-[5-[(3-fluoropyrrolidin-3-yl)methoxy]-2-methyl-4-pyridyl]pyrazolo[1,5-a]pyridin-2-yl]cyclopropanecarboxamide (Example 447, 70.0 mg, 0.171 mmol) and paraformaldehyde (27.7 mg, 0.308 mmol) in MeOH (1.5 mL). The reaction mixture was stirred at 25° C. for 30 min. Then, NaBH₃CN (23.6 mg, 0.376 mmol) was added to the reaction mixture. The reaction mixture was stirred at 25° C. for 12 hours. The organic extracts were filtered to obtain the crude product, which was purified by preparative HPLC using a YMC-Triart Prep C18 250×50 mm×10 um column (eluent: 30% to 60% (v/v) CH₃CN and H₂O with 0.04% NH₃) to afford pure product. The product was suspended in water (10 mL), the mixture frozen using dry ice/ethanol, and then lyophilized to dryness to afford the title compound as a white solid (15.2 mg, 20.7%). MS (ESI): mass calcd. for C₂₃H₂₆FN₅O₂, 423.2. m/z found, 424.3 [M+H]⁺. ¹H NMR (400 MHz, CDCl₃) δ 8.38 (br. s., 1H), 8.28 (d, J=7.6 Hz, 1H), 8.24 (s, 1H), 7.68 (d, J=0.8 Hz, 1H), 7.20 (s, 1H), 7.04-6.97 (m, 2H), 4.27-4.14 (m, 2H), 2.92-2.74 (m, 3H), 2.65-2.58 (m, 1H), 2.56 (s, 3H), 2.37 (s, 3H), 2.22-2.01 (m, 2H), 1.63-1.53 (m, 1H), 1.19-1.12 (m, 2H), 0.96-0.88 (m, 2H) ¹⁹F NMR (376 MHz, CDCl₃) δ −145.74 (s, 1F)

### Example 450: (*S)—N-[5-[5-[(1,2-dimethylazetidin-2-yl)methoxy]-2-methyl-4-pyridyl]pyrazolo[1,5-a]pyridin-2-yl]cyclopropanecarboxamide

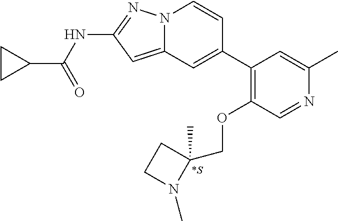

Step A. The title compound was prepared as described in Example 203 where tert-butyl 2-methyl-2-((tosyloxy)methyl)azetidine-1-carboxylate was used instead of (3-hydroxytetrahydrofuran-3-yl)methyl 4-methylbenzenesulfonate. The mixture was filtered, and the filtrate was purified by preparative HPLC using a Boston Prime C18 150×30 mm×5 um column (eluent: 45% to 75% (v/v) CH₃CN and H₂O with 0.04% NH₃) to afford pure product. The pure was further purified by SFC over DAICEL CHIRALPAK IG (250 mm×50 mm, 10 um), (eluent: 50% to 50% (v/v) supercritical CO₂ in EtOH and H₂O with 0.1% NH₃). The pure fractions were collected and the volatiles were removed under reduced pressure. The product was suspended in water (10 mL), the mixture frozen using dry ice/ethanol, and then lyophilized to dryness to afford white solid *S (42 mg, 5.27%).

Step B. (*S)—(R)—N-(5-(2-methyl-5-((2-methylazetidin-2-yl)methoxy)pyridin-4-yl)pyrazolo[1,5-a]pyridin-2-yl)cyclopropanecarboxamide. The title compound was prepared by the similar method of Example 449 Step B (18.6 mg, 119%). MS (ESI): mass calcd. for C₂₃H₂₇N₅O₂, 405.2. m/z found, 406.3 [M+H]⁺. ¹H NMR (400 MHz, CDCl₃) δ 8.28-8.23 (m, 3H), 7.70 (d, J=0.8 Hz, 1H), 7.15 (s, 1H), 6.98 (s, 1H), 6.95 (dd, J=2.0, 7.2 Hz, 1H), 3.99-3.91 (m, 2H), 3.37-3.30 (m, 1H), 3.04-2.96 (m, 1H), 2.55 (s, 3H), 2.19-2.10 (m, 4H), 1.80-1.72 (m, 1H), 1.60-1.53 (m, 1H), 1.24 (s, 3H), 1.17-1.12 (m, 2H), 0.95-0.88 (m, 2H).

### Example 451: N-[5-[5-[[(2R)-1-ethylazetidin-2-yl]methoxy]-2-methyl-4-pyridyl]pyrazolo[1,5-a]pyridin-2-yl]cyclopropanecarboxamide

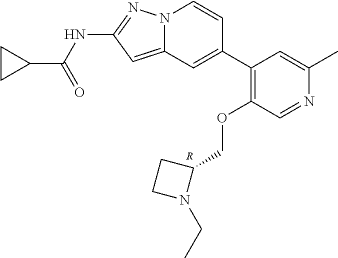

Step A: tert-butyl (R)-2-(((4-(2-(cyclopropanecarboxamido)pyrazolo[1,5-a]pyridin-5-yl)-6-methylpyridin-3-yl)oxy)methyl)azetidine-1-carboxylate. To a mixture consisting of Intermediate 7 (1.5 g, 4.9 mmol), (R)-tert-butyl 2-((tosyloxy)methyl)azetidine-1-carboxylate (1.66 g, 4.9 mmol) and DMF (25 mL) was added Cs₂CO₃ (4.76 g, 14.6 mmol) at 30° C. The reaction mixture was stirred at 75° C. for 16 hrs. The reaction was cooled to room temperature, diluted with water and extracted with EtOAc. The combined organics were dried with MgSO₄, filtered and concentrated in vacuo. The reactant mixture was purified by preparative HPLC using a YMC-Triart Prep C18 150×40 mm×7 um (eluent: 48% to 58% (v/v) CH₃CN and H₂O with 0.04% NH₃) to afford pure product. The product was suspended in water (10 mL), the mixture frozen using dry ice/ethanol, and then lyophilized to dryness to afford the title compound (850 mg, 36.59%) as a white solid.

Step B: (R)—N-(5-(5-(azetidin-2-ylmethoxy)-2-methylpyridin-4-yl)pyrazolo[1,5-a]pyridin-2-yl)cyclopropanecarboxamide. TFA (5 mL) was added to a solution consisting of (R)-tert-butyl 2-(((4-(2-(cyclopropanecarboxamido)pyrazolo[1,5-a]pyridin-5-yl)-6-methylpyridin-3-yl)oxy)methyl)azetidine-1-carboxylate (850 mg, 1.78 mmol) and 15 mL DCM. The resulting mixture was stirred at room temperature for 1 h and then the solvent was evaporated under reduced pressure to get the crude. The crude was suspended in water (10 mL), the mixture frozen using dry ice/ethanol, and then lyophilized to dryness to afford the title compound (950 mg, crude) as a yellow oil. The crude oil was used as is in the following reaction.

Step C: N-[5-[5-[[(2R)-1-ethylazetidin-2-yl]methoxy]-2-methyl-4-pyridyl]pyrazolo[1,5-a]pyridin-2-yl]cyclopropanecarboxamide The title compound was prepared as described in Example 116 step C where acetaldehyde was used instead of formaldehyde and (R)—N-(5-(5-(azetidin-2-ylmethoxy)-2-methylpyridin-4-yl)pyrazolo[1,5-a]pyridin-2-yl)cyclopropanecarboxamide was used instead of 5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine (26.1 mg, 24%). MS (ESI): mass calcd. for C₂₃H₂₇N₅O₂, 405.2. m/z found, 406.1 [M+H]⁺. ¹H NMR (400 MHz, CDCl₃) δ 8.33 (s, 1H), 8.28-8.22 (m, 2H), 7.71-7.66 (m, 1H), 7.16 (s, 1H), 6.99 (s, 1H), 6.95 (dd, J=2.0, 7.2 Hz, 1H), 4.17-4.03 (m, 2H), 3.47-3.34 (m, 2H), 2.82-2.73 (m, 1H), 2.67-2.58 (m, 1H), 2.55 (s, 3H), 2.36-2.26 (m, 1H), 2.11-1.96 (m, 2H), 1.62-1.53 (m, 1H), 1.18-1.11 (m, 2H), 0.97-0.86 (m, 5H)

### Example 452: N-[5-[2-methyl-5-[[(2R)-1-(2,2,2-trifluoroethyl)azetidin-2-yl]methoxy]-4-pyridyl]pyrazolo[1,5-a]pyridin-2-yl]cyclopropanecarboxamide

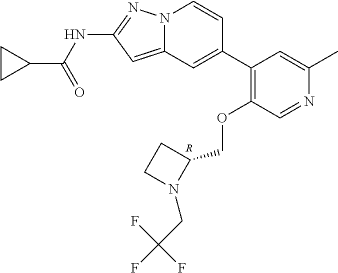

A mixture of consisting (R)—N-(5-(5-(azetidin-2-ylmethoxy)-2-methylpyridin-4-yl)pyrazolo[1,5-a]pyridin-2-yl)cyclopropanecarboxamide (Example 451 Steps A-B, 100 mg, 0.203 mmol), 2,2,2-trifluoroethyl trifluoromethanesulfonate (56.7 mg, 0.244 mmol), DIEA (78.9 mg, 0.610 mmol) and DMF (4 mL) was stirred at r.t. for 4 h. The mixture was purified by preparative HPLC with Phenomenex Gemini-NX C18 75×30 mm×3 um, column (eluent: 35% to 65% (v/v) CH₃CN and H₂O with 0.04% NH₃-H₂O+10 mM NH₄HCO₃) to afford product. The product was suspended in water (10 mL), the mixture frozen using dry ice/ethanol, and then lyophilized to dryness to afford the title compound as a white solid (8.9 mg, 9.5%). MS (ESI): mass calcd. for C₂₃H₂₄F₃N₅O₂, 459.2. m/z found, 460.2 [M+H]⁺. ¹H NMR (400 MHz, CDCl₃) δ 8.33-8.20 (m, 3H), 7.66 (s, 1H), 7.17 (s, 1H), 7.00 (s, 1H), 6.94 (dd, J=2.0, 7.6 Hz, 1H), 4.16-4.05 (m, 2H), 3.72-3.53 (m, 2H), 3.14-3.02 (m, 2H), 2.95-2.83 (m, 1H), 2.57 (s, 3H), 2.16-2.06 (m, 2H), 1.63-1.52 (m, 1H), 1.20-1.10 (m, 2H), 0.97-0.88 (m, 2H). ¹⁹F NMR (376 MHz, CDCl₃) δ −71.02, −71.14 (m, 3F)

### Example 453: (*S, *S)—N-[5-[5-(2-ethylazetidin-3-yl)oxy-2-methyl-4-pyridyl]pyrazolo[1,5-a]pyridin-2-yl]cyclopropanecarboxamide

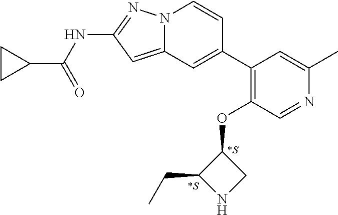 [SMILES: CC[C@@H]1NC[C@@H]1Oc1cnc(C)cc1-c1ccn2nc(NC(=O)C3CC3)cc2c1]

Step A. N-(5-(5-(((2R,3R)-1-benzhydryl-2-ethylazetidin-3-yl)oxy)-2-methylpyridin-4-yl)pyrazolo[1,5-a]pyridin-2-yl)cyclopropanecarboxamide. The title compound was prepared by the similar method of Example 136 using (cis)-1-benzhydryl-2-ethylazetidin-3-ol instead of endo-7-hydroxy-3-oxa-9-azabicyclo[3.3.1]nonane hydrochloride (1000 mg, 77%).

Step B. N-(5-(5-(((2*S,3*S)-2-ethylazetidin-3-yl)oxy)-2-methylpyridin-4-yl)pyrazolo[1,5-a]pyridin-2-yl)cyclopropanecarboxamide. A mixture of dry Pd/C (0.946 g, 10 wt. %, 8.89 mmol) and MeOH (15 mL) was added a mixture of HCO₂NH₄ (1.36 mg, 21.5 mmol) and N-(5-(5-(((cis)-1-benzhydryl-2-methylazetidin-3-yl)oxy)-2-methylpyridin-4-yl)pyrazolo[1,5-a]pyridin-2-yl)cyclopropanecarboxamide (1.0 g, 1.4 mmol) in MeOH (15 mL) and THF (30 mL), then the reaction was stirred at 50° C. for 16 h. The suspension was filtered through a pad of Celite® and the pad washed with MeOH (400 mL). The filtrate was concentrated to dryness under reduced pressure to afford the crude product. The post chromatographic product was further purified by FCC (eluent: methylene chloride:MeOH=1:0 to 5:1) to afford a yellow solid (130 mg, 22%). The racemate was further purified by SFC over DAICEL CHIRALPAK AD 250 mm×30 mm×10 μm (eluent: 55% to 55% (v/v) supercritical CO₂ in EtOH and H₂O with 0.1% NH₃). The pure fractions were collected, and the volatiles were removed under reduced pressure. The product was suspended in water (10 mL), the mixture frozen using dry ice/ethanol, and then lyophilized to dryness to afford a white powder. The white powder was not pure. It was further purified by preparative HPLC with an Agela ASB 150 mm×25 mm×5 μm column (eluent: 5% to 35 (v/v) CH₃CN and H₂O aqueous HCl (0.006)) to afford the title compound as yellow solids (3.1 mg, 10%). MS (ESI): mass calcd. for C₂₂H₂₅N₅O₂, 391.2. m/z found, 392.1 [M+H]⁺. ¹H NMR (400 MHz, DMSO-d₆) δ 11.19 (s, 1H), 9.90-9.57 (m, 1H), 9.42 (s, 1H), 8.67 (d, J=7.6 Hz, 1H), 8.35 (s, 1H), 8.08 (s, 1H), 7.84 (s, 1H), 7.20 (dd, J=1.6 Hz, J=7.6 Hz, 1H), 7.00 (s, 1H), 5.35-5.24 (m, 1H), 4.75-4.60 (m, 1H), 4.54-4.39 (m, 1H), 3.94-3.81 (m, 1H), 2.64 (s, 3H), 2.02-1.73 (m, 3H), 0.88-0.78 (m, 7H).

### Example 454: (*R)—N-[5-[2-methyl-5-[(2R)-3,3,3-trifluoro-2-hydroxy-propoxy]-4-pyridyl]pyrazolo[1,5-a]pyridin-2-yl]cyclopropanecarboxamide

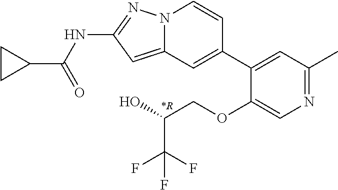 [SMILES: Cc1cc(-c2ccn3nc(NC(=O)C4CC4)cc3c2)c(OC[C@@H](O)C(F)(F)F)cn1]

The title compound was prepared by the similar method of Example 136 using (RS)-3,3,3-trifluoropropane-1,2-diol instead of endo-7-hydroxy-3-oxa-9-azabicyclo[3.3.1]nonane hydrochloride (40 mg, 83%). (RS)—N-(5-(2-methyl-5-(3,3,3-trifluoro-2-hydroxypropoxy)pyridin-4-yl)pyrazolo[1,5-a]pyridin-2-yl)cyclopropanecarboxamide (40 mg, 0.079 mmol) was further purified by SFC over DAICEL CHIRALCEL OD-H 250 mm×30 mm, 10 μm (eluent: 35% to 35% (v/v) supercritical CO₂ in EtOH and H₂O with 0.1% NH₃). The pure fractions were collected, and the volatiles were removed under reduced pressure. The product was suspended in water (10 mL), the mixture frozen using dry ice/ethanol, and then lyophilized to afford a white powder (4.8 mg, 14%). MS (ESI): mass calcd. for C₂₀H₁₉F₃N₄O₃, 420.1. m/z found, 421.2 [M+H]⁺. ¹H NMR (400 MHz, DMSO-d₆) δ 11.10 (s, 1H), 8.54 (d, J=7.2 Hz, 1H), 8.39 (s, 1H), 7.89 (d, J=0.8 Hz, 1H), 7.39 (s, 1H), 7.09 (dd, J=2.0, 7.2 Hz, 1H), 6.89 (s, 1H), 6.80-6.60 (m, 1H), 4.47-4.38 (m, 1H), 4.38-4.31 (m, 1H), 4.28-4.18 (m, 1H), 2.47 (s, 3H), 1.98-1.89 (m, 1H), 0.88-0.78 (m, 4H). ¹⁹F NMR (376 MHz, DMSO-d₆) δ −75.89 (d, J=7.5 Hz, 3F)

### Example 455: *R, *R—N-[5-[5-(1,2-dimethylazetidin-3-yl)oxy-2-methyl-4-pyridyl]pyrazolo[1,5-a]pyridin-2-yl]cyclopropanecarboxamide

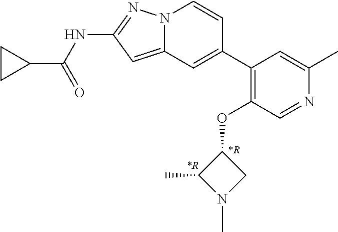 [SMILES: Cc1cc(-c2ccn3nc(NC(=O)C4CC4)cc3c2)c(O[C@@H]2CN(C)[C@@H]2C)cn1]

Step A. N-(5-(5-(((2RS,3RS)-1-benzhydryl-2-methylazetidin-3-yl)oxy)-2-methylpyridin-4-yl)pyrazolo[1,5-a]pyridin-2-yl)cyclopropanecarboxamide. The title compound was prepared by the similar method of Example 136 using Racemic-(cis)-1-benzhydryl-2-methylazetidin-3-ol instead of endo-7-hydroxy-3-oxa-9-azabicyclo[3.3.1]nonane hydrochloride (460 mg, 30%).

Step B. *R, *R—N-(5-(2-methyl-5-(((2R,3R)-2-methylazetidin-3-yl)oxy)pyridin-4-yl)pyrazolo[1,5-a]pyridin-2-yl)cyclopropanecarboxamide. A mixture of 10% Pd/C (500 mg, dry) and MeOH (4 mL) was added a mixture of HCO₂NH₄ (640 mg, 10.2 mmol) and N-(5-(5-(((2R,3R)-1-benzhydryl-2-methylazetidin-3-yl)oxy)-2-methylpyridin-4-yl)pyrazolo[1,5-a]pyridin-2-yl)cyclopropanecarboxamide (460 mg, 0.846 mmol) in THF (4 mL), then the reaction was stirred at 50° C. for 16 h. The suspension was filtered through a pad of Celite® and the pad washed with ethyl acetate (20 mL). The filtrate was concentrated to dryness under reduced pressure to afford the crude product, which was purified by FCC (DCM:MeOH=1:0 to 0:1) to afford the title compound as an off-white solid (120 mg, 38%). This racemate was separated (120 mg) was further purified by SFC over AD 250 mm×50 mm, 10 μm (eluent: 50% to 50% (v/v) supercritical CO₂ in EtOH and H₂O with 0.1% NH₃). The pure fractions were collected, and the volatiles were removed under reduced pressure. The product was suspended in water (10 mL), the mixture frozen using dry ice/acetone, and then lyophilized to dryness to afford *R, *R-isomer (50 mg) and *S, *S-isomer (50 mg). The *R, *R-isomer was pure. *S, *Sisomer (50 mg) was purified by preparative HPLC using a Gemini C18, 250×50 mm×10 μm column (eluent: 0% to 60% (v/v) CH₃CN and H₂O with 10 mM NH₄HCO₃) to afford pure product. The product was suspended in water (10 mL), the mixture frozen using dry ice/acetone, and then lyophilized to dryness to afford the title compound (13 mg, 11%) as light a yellow solid. MS (ESI): mass calcd. for C₂₁H₂₃N₅O₂, 377.4. m/z found, 378.2 [M+H]+. ¹H NMR (400 MHz, CD₃OD) δ 8.44 (d, J=7.2 Hz, 1H), 7.95 (s, 1H), 7.80 (d, J=1.2 Hz, 1H), 7.41 (s, 1H), 7.08 (dd, J=1.6, 7.2 Hz, 1H), 6.92 (s, 1H), 5.25-5.12 (m, 1H), 4.64-4.50 (m, 1H), 4.24-4.14 (m, 1H), 3.82-3.67 (m, 1H), 2.53 (s, 3H), 1.96-1.81 (m, 1H), 1.36 (d, J=6.8 Hz, 3H), 1.03-0.97 (m, 2H), 0.94-0.86 (m, 2H)

Step C. N-(5-(5-(((2R,3R)-1,2-dimethylazetidin-3-yl)oxy)-2-methylpyridin-4-yl)pyrazolo[1,5-a]pyridin-2-yl)cyclopropanecarboxamide. H(CHO)n (2.17 mg, 0.0240 mmol) was added to a solution consisting of N-(5-(2-methyl-5-(((2R,3R)-2-methylazetidin-3-yl)oxy)pyridin-4-yl)pyrazolo[1,5-a]pyridin-2-yl)cyclopropanecarboxamide (7.0 mg, 0.019 mmol) and MeOH (1 mL). The reaction mixture was stirred at room-temperature for 30 min. Then NaBH₃CN (2.3 mg, 0.037 mmol) was added to the reaction mixture. The post chromatographic product was further purified by preparative HPLC using a Phenomenex Gemini 150×25 mm×10 μm (eluent: 25% to 55% (v/v) CH₃CN and H₂O with 0.05% NH₃) to afford pure product. The product was suspended in water (10 mL), the mixture frozen using dry ice/5 ethyl alcohol, and then lyophilized to dryness to afford the title compound as a white solid (3.5 mg, 46%). MS (ESI): mass calcd. for C₂₂H₂₅N₅O₂, 391.2. m/z found, 392.3 [M+H]⁺. ¹H NMR (400 MHz, DMSO-d₆) δ 11.11 (s, 1H), 8.60 (d, J=7.2 Hz, 1H), 8.01 (s, 1H), 7.89 (s, 1H), 7.38 (s, 1H), 7.11 (dd, J=1.6, 7.6 Hz, 1H), 6.89 (s, 1H), 5.02-4.93 (m, 1H), 3.31-3.25 (m, 2H), 3.20-3.10 (m, 1H), 2.46 (s, 3H), 2.21 (s, 3H), 1.98-1.89 (m, 1H), 1.05 (d, J=6.4 Hz, 3H), 0.87-0.78 (m, 4H).

### Example 456: (*S, *S)—N-[5-[5-(1,2-dimethylazetidin-3-yl)oxy-2-methyl-4-pyridyl]pyrazolo[1,5-a]pyridin-2-yl]cyclopropanecarboxamide

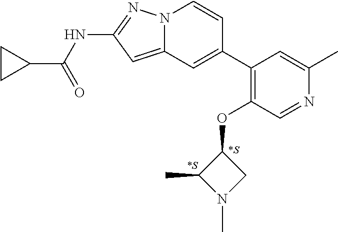 [SMILES: Cc1cc(-c2ccn3nc(NC(=O)C4CC4)cc3c2)c(O[C@H]2CN(C)[C@H]2C)cn1]

The title compound was prepared by the similar method of Example 455. The isomer (*S, *S-isomer) was used for step C (36 mg, 43%). MS (ESI): mass calcd. for C₂₂H₂₅N₅O₂, 391.2. m/z found, 392.3 [M+H]⁺. ¹H NMR (400 MHz, CD₃OD) δ 8.42 (d, J=7.2 Hz, 1H), 7.89 (s, 1H), 7.86 (s, 1H), 7.39 (s, 1H), 7.13 (dd, J=1.6, 7.2 Hz, 1H), 6.92 (s, 1H), 5.00-4.94 (m, 1H), 3.63-3.55 (m, 1H), 3.51-3.46 (m, 1H), 3.37-3.32 (m, 1H), 2.52 (s, 3H), 2.35 (s, 3H), 1.91-1.83 (m, 1H), 1.18 (d, J=6.4 Hz, 3H), 1.03-0.97 (m, 2H), 0.93-0.86 (m, 2H).

### Example 457: N-[5-[5-[[1-(2-fluoroethyl)azetidin-3-yl]methoxy]-2-methyl-4-pyridyl]pyrazolo[1,5-a]pyridin-2-yl]cyclopropanecarboxamide

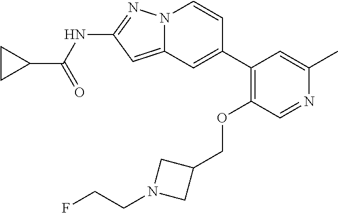 [SMILES: Cc1cc(-c2ccn3nc(NC(=O)C4CC4)cc3c2)c(OCC2CN(CCF)C2)cn1]

Step A. tert-butyl 3-4(4-(2-(cyclopropanecarboxamido)pyrazolo[1,5-a]pyridin-5-yl)-6-methylpyridin-3-yl)oxy)methyl)azetidine-1-carboxylate. The title compound was prepared by the similar method of Example 446. The tert-butyl 3-(hydroxymethyl)azetidine-1-carboxylate was used instead of (3S,5R)-5-(trifluoromethyl)piperidin-3-ol (200 mg, 50%). MS (ESI): mass calcd. for C₂₆H₃₁N₅O₄, 477.6. m/z found, 478.3 [M+H]⁺.

Step B. N-(5-(5-((1-(2-fluoroethyl)azetidin-3-yl)methoxy)-2-methylpyridin-4-yl)pyrazolo[1,5-a]pyridin-2-yl)cyclopropanecarboxamide. The title compound was prepared by the similar method of Example 452. The 1-bromo-2-fluoroethane was used instead of 2,2,2-trifluoroethyl trifluoromethanesulfonate (20 mg, 21.8%). MS (ESI): mass calcd. for C₂₃H₂₆FN₅O₂, 423.2. m/z found, 424.1 [M+H]⁺. ¹H NMR (400 MHz, CD₃OD) δ 8.41 (d, J=7.2 Hz, 1H), 8.22 (s, 1H), 7.72 (s, 1H), 7.33 (s, 1H), 7.06-6.96 (m, 1H), 6.90 (s, 1H), 4.43-4.24 (m, 2H), 4.17 (d, J=5.2 Hz, 2H), 3.51-3.41 (m, 2H), 3.18-3.09 (m, 2H), 2.99-2.88 (m, 1H), 2.68-2.54 (m, 2H), 2.51 (s, 3H), 1.94-1.80 (m, 1H), 1.04-0.95 (m, 2H), 0.93-0.84 (m, 2H). ¹⁹F NMR (376 MHz, DMSO-d₆) δ −221.04-221.56 (m, 1F).

### Example 458: Trans-*R, *R—N-[5-[5-[(4-methoxypyrrolidin-3-yl)methoxy]-2-methyl-4-pyridyl]pyrazolo[1,5-a]pyridin-2-yl]cyclopropanecarboxamide

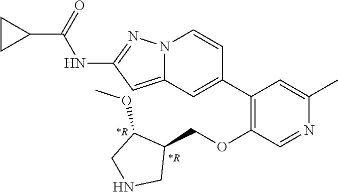 [SMILES: CO[C@H]1CNC[C@@H]1COc1cnc(C)cc1-c1ccn2nc(NC(=O)C3CC3)cc2c1]

The title compound was prepared by the similar method of Example 8 using rel-trans-tert-butyl-3-(hydroxymethyl)-4-methoxypyrrolidine-1-carboxylate instead of (3S,5R)-5-(trifluoromethyl)piperidin-3-ol and (18.7 mg, 4.8%). MS (ESI): mass calcd. for C₂₃H₂₇N₅O₃, 421.2. m/z found, 422.2 [M+H]⁺. ¹H NMR (400 MHz, CD₃OD) δ 8.45-8.41 (m, 1H), 8.25 (s, 1H), 7.78-7.68 (m, 1H), 7.36 (s, 1H), 7.02 (dd, J=7.2, 2.0 Hz, 1H), 6.90 (s, 1H), 4.16-4.05 (m, 2H), 3.81-3.75 (m, 1H), 3.19 (s, 3H), 3.17-3.08 (m, 1H), 2.95-2.80 (m, 2H), 2.64 (dd, J==11.7, 5.9 Hz, 1H), 2.55-2.46 (m, 4H), 1.93-1.82 (m, 1H), 1.05-0.97 (m, 2H), 0.93-0.85 (m, 2H).

### Example 459: Cis-rac-N-[5-[5-[(4-methoxypyrrolidin-3-yl)methoxy]-2-methyl-4-pyridyl]pyrazolo[1,5-a]pyridin-2-yl]cyclopropanecarboxamide

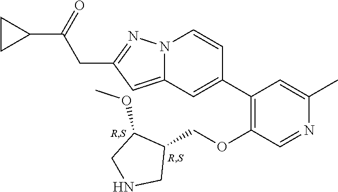 [SMILES: CO[C@H]1CNC[C@H]1COc1cnc(C)cc1-c1ccn2nc(CC(=O)C3CC3)cc2c1]

The title compound was prepared by the similar method of Example 8 using rel-cis-tert-butyl-3-(hydroxymethyl)-4-methoxypyrrolidine-1-carboxylate instead of (2S,4R)-tert-butyl 4-hydroxy-2-(trifluoromethyl)piperidine-1-carboxylate (5.8 mg, 1.47%). MS (ESI): mass calcd. for C₂₃H₂₇N₅O₃, 421.2. m/z found, 422.1 [M+H]⁺. ¹H NMR (400 MHz, DMSO-d₆) δ 11.01 (s, 1H), 8.53-8.46 (m, 1H), 8.29-8.24 (m, 1H), 7.77-7.71 (m, 1H), 7.28 (s, 1H), 6.97 (dd, J=7.3, 2.0 Hz, 1H), 6.83-6.78 (m, 1H), 4.21-4.14 (m, 1H), 4.04-3.98 (m, 1H), 3.78-3.67 (m, 1H), 3.12-3.08 (m, 3H), 2.84-2.70 (m, 3H), 2.57-2.50 (m, 1H), 2.39 (s, 3H), 2.36-2.28 (m, 1H), 1.91-1.82 (m, 1H), 0.80-0.69 (m, 4H).

### Example 460: N-[5-[2-methyl-5-[[(1S,5R)-9-methyl-3-oxa-9-azabicyclo[3.3.1]nonan-7-yl]oxy]-4-pyridyl]pyrazolo[1,5-a]pyridin-2-yl]cyclopropanecarboxamide

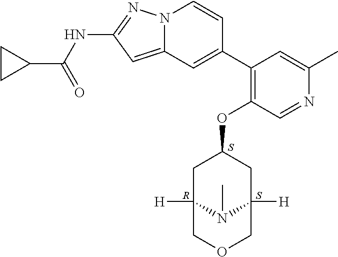

The title compound was prepared by the similar method of Example 449 using formaldehyde instead of acetaldehyde and endo-7-hydroxy-3-oxa-9-azabicyclo[3.3.1]nonane hydrochloride instead of (3S,5R)-5-(trifluoromethyl)piperidin-3-ol (42.9 mg, 40%). MS (ESI): mass calcd. for C₂₅H₂₉N₅O₃, 447.2. m/z found, 448.3 [M+H]⁺. ¹H NMR (400 MHz, DMSO-d₆) δ 11.07 (s, 1H), 8.54-8.48 (m, 1H), 8.27-8.23 (m, 1H), 8.19 (s, 1H), 7.39 (s, 1H), 7.25 (dd, J=7.3, 2.0 Hz, 1H), 6.82 (s, 1H), 4.76-4.66 (m, 1H), 3.79 (dd, J=10.7, 2.4 Hz, 2H), 3.50 (d, J=10.7 Hz, 2H), 2.65-2.59 (m, 2H), 2.45 (s, 3H), 2.43-2.35 (m, 5H), 1.98-1.85 (m, 1H), 1.63 (d, J=15.5 Hz, 2H), 0.88-0.76 (m, 4H).

### Example 461: Trans-*R, *R—N-[5-[5-[(4-methoxy-1-methyl-pyrrolidin-3-yl)methoxy]-2-methyl-4-pyridyl]pyrazolo[1,5-a]pyridin-2-yl]cyclopropanecarboxamide

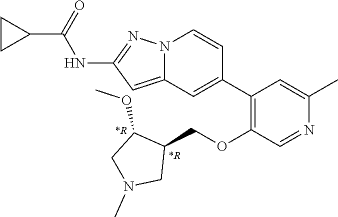 [SMILES: CO[C@H]1CN(C)C[C@@H]1COc1cnc(C)cc1-c1ccn2nc(NC(=O)C3CC3)cc2c1]

Step A. rac-N-(5-(5-(((3R,4R)-4-methoxypyrrolidin-3-yl)methoxy)-2-methylpyridin-4-yl)pyrazolo[1,5-a]pyridin-2-yl)cyclopropanecarboxamide. The title compound was prepared by the similar method of Example 458. The isomers were separated by SFC OD-H (2×25 cm) 25% methanol (0.1% DEA)/CO₂, 100 bar inj vol.: 1 mL, 4 mg/mL methanol:DCM yielded 41 mg of the second eluting peak (ee>99%). MS (ESI): mass calcd. for C₂₃H₂₇N₅O₃, 421.2. m/z found, 422.2 [M+H]⁺.

Step B. rac-N-(5-(5-(((3R,4R)-4-methoxy-1-methylpyrrolidin-3-yl)methoxy)-2-methylpyridin-4-yl)pyrazolo[1,5-a]pyridin-2-yl)cyclopropanecarboxamide. The title compound was prepared by the similar method of Example 10. MS (ESI): mass calcd. for C₂₄H₂₉N₅O₃, 435.2. m/z found, 436.2 [M+H]⁺. ¹H NMR (500 MHz, CD₃OD) δ 8.46-8.38 (m, 1H), 8.25 (s, 1H), 7.80-7.71 (m, 1H), 7.37 (s, 1H), 7.01 (dd, J=7.2, 2.0 Hz, 1H), 6.90 (s, 1H), 4.19-4.09 (m, 2H), 3.74-3.69 (m, 1H), 3.16 (s, 3H), 2.97-2.90 (m, 1H), 2.79-2.72 (m, 1H), 2.58-2.54 (m, 1H), 2.53 (s, 3H), 2.47-2.42 (m, 1H), 2.25 (s, 3H), 2.23-2.18 (m, 1H), 1.91-1.84 (m, 1H), 1.04-0.96 (m, 2H), 0.94-0.87 (m, 2H).

### Example 462: N-[5-[2-methyl-5-[[(1S,5R)-9-(trideuteriomethyl)-3-oxa-9-azabicyclo[3.3.1]nonan-7-yl]oxy]-4-pyridyl]pyrazolo[1,5-a]pyridin-2-yl]cyclopropanecarboxamide

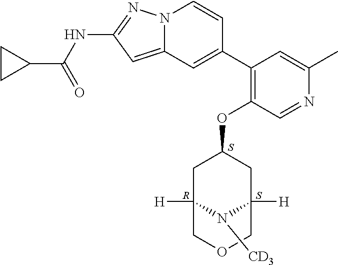

The title compound was prepared by the similar method of Example 460 using deuterio-formaldehyde instead of paraformaldehyde and sodium cyanoborodeuteride instead of sodium cyanoborohydride (53.8 mg, 51%). MS (ESI): mass calcd. for C₂₅H₂₉N₅O₃, 450.2. m/z found, 451.1 [M+H]⁺. ¹H NMR (500 MHz, DMSO-d₆) δ 11.06 (s, 1H), 8.55-8.46 (m, 1H), 8.27-8.23 (m, 1H), 8.19 (s, 1H), 7.39 (s, 1H), 7.25 (dd, J=7.3, 2.0 Hz, 1H), 6.82 (s, 1H), 4.74-4.66 (m, 1H), 3.86-3.75 (m, 2H), 3.54-3.46 (m, 2H), 2.62 (d, J=6.9 Hz, 2H), 2.45 (s, 3H), 2.43-2.34 (m, 2H), 1.99-1.89 (m, 1H), 1.63 (d, J=15.0 Hz, 2H), 0.87-0.77 (m, 4H).

### Example 463: (1R,2R)-2-methyl-N-[5-[2-methyl-5-[[(1S,4S,7R)-2-oxa-5-azabicyclo[2.2.1]heptan-7-yl]oxy]-4-pyridyl]pyrazolo[1,5-a]pyridin-2-yl]cyclopropanecarboxamide

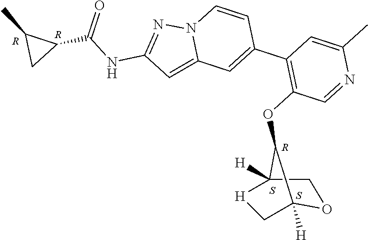

The title compound was prepared by the similar method of Example 445 using tert-butyl (1S,4S,7R)-7-hydroxy-2-oxa-5-azabicyclo[2.2.1]heptane-5-carboxylate instead of (2S,4R)-tert-butyl 4-hydroxy-2-(trifluoromethyl)piperidine-1-carboxylate and (1R,2R)—N-(5-(5-fluoro-2-methylpyridin-4-yl)pyrazolo[1,5-a]pyridin-2-yl)-2-methylcyclopropane-1-carboxamide instead of Intermediate 6 (116 mg, 88%). MS (ESI): mass calcd. for C₂₃H₂₅N₅O₃, 419.2. m/z found, 420.2 [M+H]⁺. ¹H NMR (500 MHz, CD₃OD) δ 8.42-8.37 (m, 2H), 7.77-7.74 (m, 1H), 7.40 (s, 1H), 7.04 (dd, J=7.2, 2.0 Hz, 1H), 6.89 (s, 1H), 4.87 (d, J=2.3 Hz, 1H), 4.34 (s, 1H), 4.02 (dd, J=7.8, 2.1 Hz, 1H), 3.87 (d, J=7.8 Hz, 1H), 3.55 (t, J=2.2 Hz, 1H), 3.14 (dd, J=11.2, 1.7 Hz, 1H), 2.97 (dd, J=11.2, 0.8 Hz, 1H), 2.54 (s, 3H), 1.62-1.56 (m, 1H), 1.44-1.36 (m, 1H), 1.23-1.19 (m, 1H), 1.17 (d, J=6.0 Hz, 3H), 0.75-0.71 (m, 1H).

### Example 464: N-(5-(5-((1s,3s)-3-aminocyclobutoxy)-2-methylpyridin-4-yl)pyrazolo[1,5-a]pyridin-2-yl)cyclopropanecarboxamide

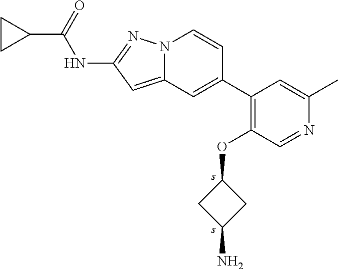 [SMILES: Cc1cc(-c2ccn3nc(NC(=O)C4CC4)cc3c2)c(O[C@H]2C[C@@H](N)C2)cn1]

The title compound was prepared by the similar method of Example 136 using cis-3-aminocyclobutanoyl hydrochloride instead of endo-7-hydroxy-3-oxa-9-azabicyclo[3.3.1]nonane hydrochloride (216 mg, 88.8%). MS (ESI): mass calcd. for C₂₁H₂₃N₅O₂, 377.2. m/z found, 378.2 [M+H]⁺. ¹H NMR (500 MHz, CDCl₃) δ 10.14 (d, J=11.7 Hz, 1H), 8.20 (d, J=7.2 Hz, 1H), 8.02 (s, 1H), 7.57 (d, J=1.9 Hz, 1H), 7.07 (s, 1H), 6.98 (s, 1H), 6.86 (dd, J=7.1, 1.9 Hz, 1H), 4.29 (p, J=6.9 Hz, 1H), 3.11 (s, 1H), 2.87 (s, 2H), 2.79 (qt, J=9.3, 6.4, 5.7 Hz, 2H), 2.44 (s, 3H), 1.77 (dt, J=12.0, 7.9 Hz, 2H), 1.56 (tt, J=8.6, 4.6 Hz, 1H), 1.05 (p, J=4.3 Hz, 2H), 0.74 (dq, J=5=7.3, 4.0 Hz, 2H).

### Example 465: N-[5-[5-[(2R)-2-amino-2-phenyl-ethoxy]-2-methyl-4-pyridyl]pyrazolo[1,5-a]pyridin-2-yl]cyclopropanecarboxamide

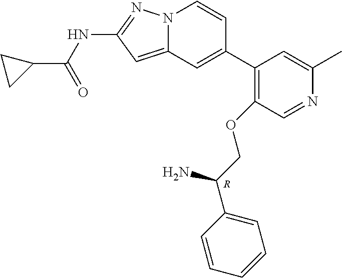

The title compound was prepared by the similar method of Example 253. The mixture of regioisomer and stereoisomers were separated by chiral IA (2×25 cm) 50% methanol(DEA)/CO₂, 100 bar, 50 mL/min, inj. Vol. 0.5 mL at 0.3 mg·mL. The first eluting peak was collected. (72 mg, 25%). MS (ESI): mass calcd. for C₂₅H₂₅N₅O₂, 427.2. m/z found, 428.2 [M+H]⁺. ¹H NMR (600 MHz, CDCl₃) δ 9.07-9.02 (m, 1H), 8.15 (d, J=5.8 Hz, 2H), 7.47 (d, J=1.9 Hz, 1H), 7.31-7.23 (m, 4H), 7.25-7.19 (m, 1H), 7.06 (s, 1H), 6.95 (s, 1H), 6.73 (dd, J=7.2, 1.9 Hz, 1H), 5.21 (s, 1H), 4.27 (dd, J=7.5, 4.6 Hz, 1H), 4.13 (dd, J=8.9, 4.6 Hz, 1H), 4.06-4.00 (m, 1H), 3.64 (q, J=7.0 Hz, 1H), 2.46 (s, 3H), 1.52 (tt, J=8.6, 4.5 Hz, 1H), 1.11-1.02 (m, 2H), 0.80 (dq, J=7.4, 4.1 Hz, 2H).

### Example 466: N-[5-[5-[(1S)-2-amino-1-phenyl-ethoxy]-2-methyl-4-pyridyl]pyrazolo[1,5-a]pyridin-2-yl]cyclopropanecarboxamide

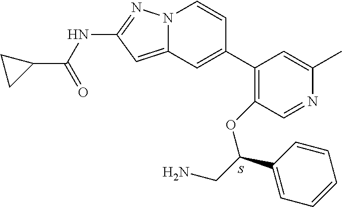

The title compound was prepared by the similar method of Example 465 using mixture of tert-butyl (S)-(2-hydroxy-2-phenylethyl)carbamate. MS (ESI): mass calcd. for C₂₅H₂₅N₅O₂, 427.2. m/z found, 428.2 [M+H]⁺. ¹H NMR (600 MHz, CDCl₃) δ 8.72 (s, 1H), 8.23 (d, J=7.1 Hz, 1H), 8.01 (s, 1H), 7.56 (d, J=1.9 Hz, 1H), 7.31-7.15 (m, 4H), 7.05 (s, 1H), 6.97 (s, 1H), 6.93 (dt, J=7.3, 4.4 Hz, 1H), 5.11-5.03 (m, 1H), 4.14 (tt, J=10.6, 5.6 Hz, 1H), 3.40 (s, 1H), 3.28 (d, J=2.5 Hz, 2H), 3.02 (dd, J=13.6, 7.1 Hz, 1H), 2.40 (s, 3H), 1.53 (s, 1H), 1.08 (p, J=4.2 Hz, 2H), 0.83 (ddt, J=13.4, 9.4, 7.2 Hz, 2H).

### Example 467: N-[5-[5-[(2S)-2-amino-2-phenyl-ethoxy]-2-methyl-4-pyridyl]pyrazolo[1,5-a]pyridin-2-yl]cyclopropanecarboxamide

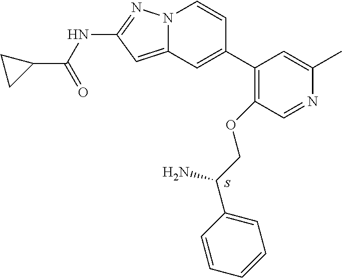

The title compound was prepared by the similar method of Example 465. This material was the second eluting peak from Example 465. MS (ESI): mass calcd. for C₂₅H₂₅N₅O₂, 427.2; m/z found, 428.2 [M+H]⁺. ¹H NMR (600 MHz, CDCl₃) δ 8.85 (s, 1H), 8.24 (d, J=5.2 Hz, 2H), 7.57 (dd, J=2.0, 0.9 Hz, 1H), 7.42-7.33 (m, 4H), 7.35-7.29 (m, 1H), 7.16 (s, 1H), 7.04 (s, 1H), 6.83 (dd, J=7.2, 1.9 Hz, 1H), 4.40-4.34 (m, 1H), 4.23 (dd, J=8.9, 4.6 Hz, 1H), 4.12 (dd, J=8.9, 7.6 Hz, 1H), 2.78 (s, OH), 2.56 (s, 3H), 1.61 (p, J=3.9 Hz, 1H), 1.24-1.15 (m, 2H), 0.91 (dq, J=7.2, 4.1 Hz, 2H).

### Example 468: N-[5-[2-methyl-5-[[(2R)-1-methylazetidin-2-yl]methoxy]-4-pyridyl]pyrazolo[1,5-a]pyridin-2-yl]cyclopropanecarboxamide

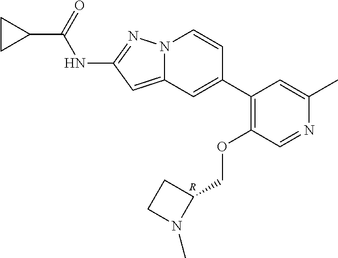

The title compound was prepared by the similar method of Example 136 using (R)-(1-methylazetidin-2-yl)methanol instead of endo-7-hydroxy-3-oxa-9-azabicyclo[3.3.1]nonane hydrochloride (333 mg, 94%). MS (ESI): mass calcd. for C₂₂H₂₅N₅O₂, 391.2. m/z found, 392.2 [M+H]⁺. ¹H NMR (500 MHz, CDCl₃) δ 9.33 (d, J=6.3 Hz, 1H), 8.31-8.24 (m, 2H), 7.72 (d, J=1.9 Hz, 1H), 7.16 (s, 1H), 7.02 (s, 1H), 6.97 (dd, J=7.3, 2.0 Hz, 1H), 4.13-4.04 (m, 2H), 3.51-3.39 (m, 1H), 3.34 (td, J=8.4, 4.1 Hz, 1H), 2.82 (td, J=8.7, 6.8 Hz, 1H), 2.55 (s, 3H), 2.27 (s, 3H), 2.08-1.97 (m, 2H), 1.59 (td, J=8.0, 4.1 Hz, 1H), 1.28-1.11 (m, 2H), 0.95-0.85 (m, 2H).

### Example 469: rac-N-[5-[5-[(3,3-difluoroazetidin-2-yl)methoxy]-2-methyl-4-pyridyl]pyrazolo[1,5-a]pyridin-2-yl]cyclopropanecarboxamide

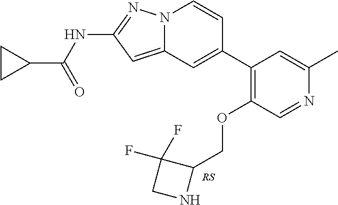

The title compound was prepared using the method described in Intermediate 10, Step A, and using Intermediate 7 instead of 6-chloro-4-iodopyridin-3-ol and using tert-butyl 3,3-difluoro-2-(hydroxymethyl)azetidine-1-carboxylate instead of tert-butyl (1R,5S,7s)-7-hydroxy-3-oxa-9-azabicyclo[3.3.1]nonane-9-carboxylate. The Boc-protecting was removed with 1:4 TFA:DCM. The crude was purified via prep. HPLC using 10-60% ACN-H₂O with 20 mM of aq. NH₄OH as modifier. The pure fraction was collected, froze, and lyophilized down to afford a white powder (190 mg, 70%). MS (ESI): mass calcd. for C₂₁H₂₁F₂N₅O₂, 413.7. m/z found, 414.2 [M+H]⁺. 1H NMR (400 MHz, CD₃OD) δ 8.34-8.28 (m, 1H), 8.18 (s, 1H), 7.70 (dd, J=2.0, 0.9 Hz, 1H), 7.30 (s, 1H), 6.96 (dd, J=7.3, 2.0 Hz, 1H), 6.80 (s, 1H), 4.40-4.23 (m, 2H), 4.17 (dd, J=9.8, 5.6 Hz, 1H), 3.82 (td, J=13.9, 11.1 Hz, 1H), 3.62 (q, J=12.1 Hz, 1H), 2.45 (s, 3H), 1.78 (s, 1H), 0.96-0.87 (m, 2H), 0.81 (dt, J=8.0, 3.2 Hz, 2H).

### Example 470: N-[5-[2-methyl-5-[(3-phenylazetidin-3-yl)methoxy]-4-pyridyl]pyrazolo[1,5-a]pyridin-2-yl]cyclopropanecarboxamide

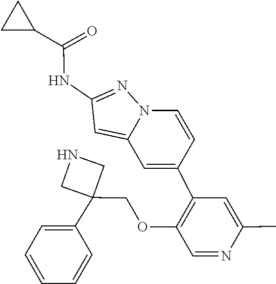 [SMILES: Cc1cc(-c2ccn3nc(NC(=O)C4CC4)cc3c2)c(OCC2(c3ccccc3)CNC2)cn1]

The title compound was prepared by the similar method of Example 136 using (3-phenylazetidin-3-yl)methanol hydrochloride instead of endo-7-hydroxy-3-oxa-9-azabicyclo[3.3.1]nonane hydrochloride (161 mg, 71%). MS (ESI): mass calcd. for C₂₇H₂₇N₅O₂, 453.2. m/z found, 454.2 [M+H]⁺. 1H NMR (400 MHz, CD₃OD) δ 8.35 (d, J=7.2 Hz, 1H), 8.23 (s, 1H), 7.53 (d, J=1.9 Hz, 1H), 7.30 (s, 1H), 7.27-7.12 (m, 4H), 7.11-7.03 (m, 2H), 6.91 (s, 1H), 6.79 (dd, J=7.2, 1.9 Hz, 1H), 4.52 (s, 2H), 4.05-3.62 (m, 4H), 2.50 (s, 3H), 1.96-1.82 (m, 1H), 1.08-0.98 (m, 2H), 0.98-0.86 (m, 2H).

### Example 471: N-[5-[5-[[(2S)-3,3-dimethylazetidin-2-yl]methoxy]-2-methyl-4-pyridyl]pyrazolo[1,5-a]pyridin-2-yl]cyclopropanecarboxamide

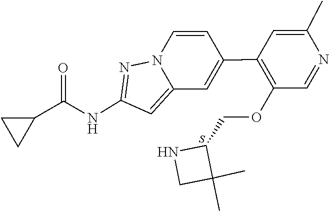

The title compound was prepared by the similar method of Example 8 using tert-butyl (S)-2-(hydroxymethyl)-3,3-dimethylazetidine-1-carboxylate instead of (2S,4R)-tert-butyl 4-hydroxy-2-(trifluoromethyl)piperidine-1-carboxylate (155 mg, 76%). MS (ESI): mass calcd. for C₂₃H₂₇N₅O₂, 405.2. m/z found, 406.1 [M+H]⁺. 1H NMR (400 MHz, DMSO-d₆) δ 11.07 (s, 1H), 8.55 (dt, J=7.2, 1.0 Hz, 1H), 8.36 (s, 1H), 7.81 (dd, J=2.0, 0.9 Hz, 1H), 7.35 (s, 1H), 7.02 (dd, J=7.2, 2.0 Hz, 1H), 6.89-6.83 (m, 1H), 4.12 (ddd, J=30.8, 9.7, 6.6 Hz, 2H), 3.73 (t, J=6.6 Hz, 1H), 3.24 (d, J=7.0 Hz, 1H), 2.93 (d, J=7.0 Hz, 1H), 2.46 (s, 3H), 1.98-1.86 (m, 1H), 1.10 (s, 3H), 1.08 (s, 3H), 0.89-0.74 (m, 4H).

### Example 472: Racemic-N-[5-[2-methyl-5-(7-oxa-2-azaspiro[3.5]nonan-3-ylmethoxy)-4-pyridyl]pyrazolo[1,5-a]pyridin-2-yl]cyclopropanecarboxamide

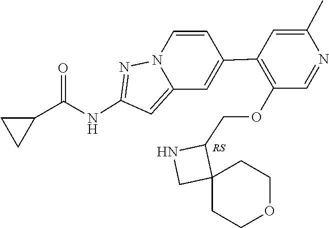 [SMILES: Cc1cc(-c2ccn3nc(NC(=O)C4CC4)cc3c2)c(OCC2NCC23CCOCC3)cn1]

The title compound was prepared by the similar method of Example 8 using tert-butyl (RS)-2-(hydroxymethyl)-3,3-dimethylazetidine-1-carboxylate instead of (2S,4R)-tert-butyl 4-hydroxy-2-(trifluoromethyl)piperidine-1-carboxylate (155 mg, 76%). MS (ESI): mass calcd. for C₂₅H₂₉N₅O₃, 447.2. m/z found, 448.3 [M+H]⁺. 1H NMR (400 MHz, DMSO-d₆) δ 11.06 (s, 1H), 8.59-8.47 (m, 1H), 8.37 (s, 1H), 7.83 (dd, J=2.1, 0.9 Hz, 1H), 7.35 (s, 1H), 7.03 (dd, J=7.3, 2.0 Hz, 1H), 6.88 (s, 1H), 4.25 (dd, J=9.8, 6.7 Hz, 1H), 4.11 (dd, J=9.8, 6.4 Hz, 1H), 3.74 (t, J==6.5 Hz, 1H), 3.67-3.54 (m, 2H), 3.28-3.16 (m, 4H), 2.46 (s, 3H), 1.97-1.87 (m, 1H), 1.76-1.58 (m, 4H), 0.87-0.75 (m, 4H).

### Example 473: N-[5-[2-methyl-5-[[(2S)-1,3,3-trimethylazetidin-2-yl]methoxy]-4-pyridyl]pyrazolo[1,5-a]pyridin-2-yl]cyclopropanecarboxamide

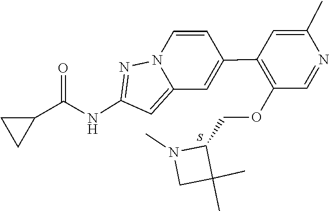

The title compound (29 mg, 73.7%) was prepared by the similar method of Example 8 using tert-butyl (S)-2-(hydroxymethyl)-3,3-dimethylazetidine-1-carboxylate instead of (2S,4R)-tert-butyl 4-hydroxy-2-(trifluoromethyl)piperidine-1-carboxylate. The methylation step followed a similar method of Example 10. MS (ESI): mass calcd. for C₂₄H₂₉N₅O₂, 419.2. m/z found, 420.3 [M+H]⁺. 1H NMR (500 MHz, DMSO-d₆) δ 11.06 (s, 1H), 8.55 (dt, J=7.3, 0.9 Hz, 1H), 8.35 (s, 1H), 7.80 (dd, J=2.0, 0.9 Hz, 1H), 7.34 (s, 1H), 7.00 (dd, J=7.2, 2.0 Hz, 1H), 6.87-6.82 (m, 1H), 4.15-4.05 (m, 2H), 3.01 (d, J=5.6 Hz, 1H), 2.93 (t, J=6.4 Hz, 1H), 2.50-2.48 (m, 1H), 2.46 (s, 3H), 2.17 (s, 3H), 1.97-1.87 (m, 1H), 1.11 (s, 3H), 1.00 (s, 3H), 0.87-0.77 (m, 4H).

### Example 474: N-[5-[5-methyl-2-[[(2S)-morpholin-2-yl]methoxy]-3-pyridyl]pyrazolo[1,5-a]pyridin-2-yl]cyclopropanecarboxamide

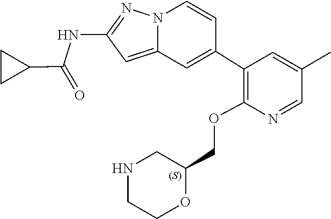

The title compound was prepared using methods analogous to those described in Example 136. MS (ESI): mass calcd. for C₂₂H₂₅N₅O₃, 407.2. m/z found, 408.2 [M+H]⁺. ¹H NMR (400 MHz, CDCl₃) δ 8.41 (br. s., 1H), 8.25 (d, J=7.2 Hz, 1H), 7.96 (d, J=1.6 Hz, 1H), 7.65 (s, 1H), 7.52 (d, J=2.0 Hz, 1H), 7.01 (dd, J=1.8, 7.2 Hz, 1H), 6.94 (s, 1H), 4.37 (d, J=5.2 Hz, 2H), 3.98-3.84 (m, 2H), 3.74-3.62 (m, 1H), 3.05-2.74 (m, 4H), 2.31 (s, 3H), 1.63-1.51 (m, 1H), 1.20-1.08 (m, 2H), 0.96-0.83 (m, 2H).

### Example 475: 3-[[6-(difluoromethoxy)-4-[2-([1,2,4]triazolo[1,5-a]pyridin-7-ylamino)pyrazolo[1,5-a]pyridin-5-yl]-3-pyridyl]oxy]-2,2-dimethyl-propanenitrile

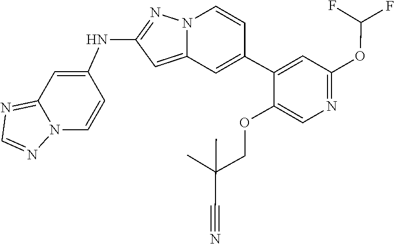 [SMILES: CC(C)(C#N)COc1cnc(OC(F)F)cc1-c1ccn2nc(Nc3ccn4ncnc4c3)cc2c1]

The title compound was prepared using a similar method described in Example 682, except tBuXPhos Pd G3 was used as the catalyst. MS (ESI): mass calcd. for C₂₄H₂₀F₂N₈O₂, 490.2. m/z found, 491.1 [M+H]⁺. ¹H NMR (500 MHz, DMSO-d₆) δ 9.83 (s, 1H), 8.73 (dd, J=7.2, 1.0 Hz, 1H), 8.70 (dd, J=7.4, 0.7 Hz, 1H), 8.24 (s, 1H), 8.15 (s, 1H), 8.12 (dd, J=2.4, 0.7 Hz, 1H), 7.90 (dd, J=2.1, 0.9 Hz, 1H), 7.84-7.50 (m, 1H), 7.28 (d, J=1.4 Hz, 1H), 7.07-7.01 (m, 2H), 6.27 (d, J=0.9 Hz, 1H), 4.21 (s, 2H), 1.36 (s, 6H).

### Example 476: N-[5-[5-cyano-2-[[(3R)-pyrrolidin-3-yl]methoxy]phenyl]pyrazolo[1,5-a]pyridin-2-yl]cyclopropanecarboxamide

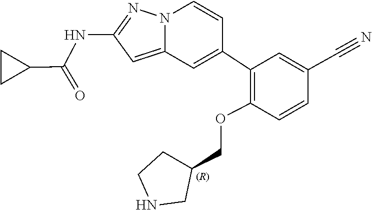

The title compound was prepared using methods analogous to those described in Example 100. MS (ESI): mass calcd. for C₂₃H₂₃N₅O₂, 401.2. m/z found, 402.2 [M+H]⁺. ¹H NMR (400 MHz, DMSO-d₆) δ 11.07 (s, 1H), 8.58-8.50 (m, 1H), 7.91-7.88 (m, 1H), 7.87-7.83 (m, 1H), 7.75-7.69 (m, 1H), 7.38-7.27 (m, 1H), 6.99 (dd, J=7.2, 2.0 Hz, 1H), 6.85 (s, 1H), 4.16-3.98 (m, 2H), 2.85-2.52 (m, 4H), 2.42-2.32 (m, 1H), 1.97-1.88 (m, 1H), 1.83-1.72 (m, 1H), 1.45-1.33 (m, 1H), 0.88-0.76 (m, 4H).

### Example 477: (2*R, 1*S)—N-[5-[5-cyano-2-(1-morpholin-2-ylethoxy)phenyl]pyrazolo[1,5-a]pyridin-2-yl]cyclopropanecarboxamide

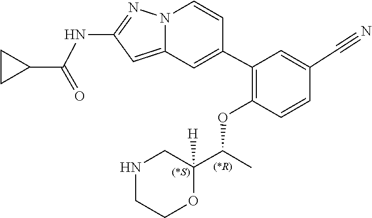 [SMILES: C[C@@H](Oc1ccc(C#N)cc1-c1ccn2nc(NC(=O)C3CC3)cc2c1)[C@@H]1CNCCO1]

The title compound was prepared using synthetic methods analogous to those described in Example 100 and chiral separation methods similar to those in Example 5. MS (ESI): mass calcd. for C₂₄H₂₅N₅O₃, 431.2. m/z found, 431.9 [M+H]⁺. ¹H NMR (500 MHz, DMSO-d₆) δ 11.07 (s, 1H), 8.54 (d, J=7.2 Hz, 1H), 7.90 (d, J=2.2 Hz, 1H), 7.85-7.77 (m, 2H), 7.34 (d, J=8.8 Hz, 1H), 7.06 (dd, J=7.2, 2.0 Hz, 1H), 6.85 (s, 1H), 4.68-4.59 (m, 1H), 3.85-3.71 (m, 1H), 3.52-3.38 (m, 2H), 2.80-2.69 (m, 1H), 2.69-2.52 (m, 2H), 1.98-1.89 (m, 1H), 1.20 (d, J=6.3 Hz, 3H), 1.03-0.95 (m, 1H), 0.86-0.77 (m, 4H).

### Example 478: 4-[[(2R)-morpholin-2-yl]methoxy]-3-[2-(2-pyridylamino)pyrazolo[1,5-a]pyridin-5-yl]benzonitrile

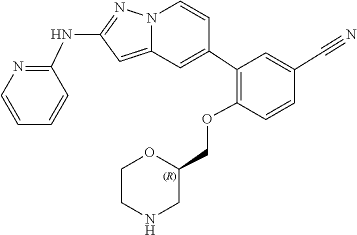

The title compound was prepared using a similar coupling method described in Example 198, followed by a Boc-deprotection step similar to that described in Example 9, Step B. MS (ESI): mass calcd. for C₂₄H₂₂N₆O₂, 426.2. m/z found, 427.2 [M+H]⁺. ¹H NMR (400 MHz, CD₃CN) δ 8.37-8.32 (m, 1H), 8.25-8.21 (m, 1H), 7.97 (br. s., 1H), 7.79 (d, J=2.0 Hz, 1H), 7.72 (dd, J=2.0, 8.8 Hz, 1H), 7.67-7.61 (m, 2H), 7.40-7.36 (m, 1H), 7.18 (d, J=8.4 Hz, 1H), 6.94 (dd, J=1.6, 7.2 Hz, 1H), 6.86-6.80 (m, 1H), 6.76 (s, 1H), 4.13-4.03 (m, 2H), 3.82-3.77 (m, 1H), 3.77-3.70 (m, 1H), 3.57-3.46 (m, 1H), 2.88-2.82 (m, 1H), 2.73-2.68 (m, 2H), 2.64-2.56 (m, 1H)

### Example 479: N-[5-[2-[[(1R,5S)-3-oxa-9-azabicyclo[3.3.1]nonan-7-yl]oxy]-5-prop-1-ynyl-phenyl]pyrazolo[1,5-a]pyridin-2-yl]cyclopropanecarboxamide

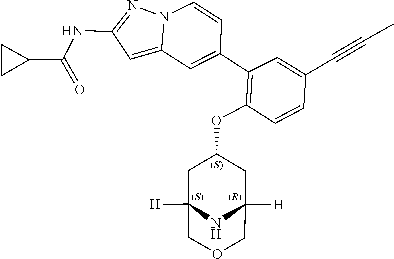

Step A: N-(5-(2-fluoro-5-(prop-1-yn-1-yl)phenyl)pyrazolo[1,5-a]pyridin-2-yl)cyclopropanecarboxamide. To a 10 mL vial charged with N-(5-bromopyrazolo[1,5-a]pyridin-2-yl)cyclopropanecarboxamide (Intermediate 2, 303 mg, 1.08 mmol), (2-fluoro-5-(prop-1-yn-1-yl)phenyl)boronic acid (244 mg, 1.37 mmol), chloro(2-dicyclohexylphosphino-2′,6′-diisopropoxy-1,1′biphenyl)[2-(2′-amino-1,1′-biphenyl)]palladium(II) (20 mg, 0.026 mmol), was added 0.5 M tribasic potassium phosphate (2.8 mL) and dioxane (3.6 mL). The vial was evacuated and backfilled 2× with nitrogen. The reaction mixture was placed on a preheated 90° C. heating block for 60 min. Then, the reaction mixture was allowed to cool to room temperature. The reaction mixture was diluted with EtOAc (20 mL) then the mixture was passed through a pad of 1:1 celite: MgSO₄. The pad was rinsed with EtOAc (2×15 mL). The combined organics were concentrated, then purified (FCC, SiO₂, 0-35% EtOAc/hexanes) to provide the title compound (306 mg, 94% purity, 80% yield). MS (ESI): mass calculated (calcd.) for C₂₀H₁₆FN₃O, 333.1. m/z found, 334.2 [M+H]⁺. ¹H NMR (400 MHz, DMSO-d₆) δ 11.08 (s, 1H), 8.58 (dt, J=7.2, 1.0 Hz, 1H), 7.79 (dt, J=2.1, 1.1 Hz, 1H), 7.64 (dd, J=7.6, 2.2 Hz, 1H), 7.50-7.43 (m, 1H), 7.33 (dd, J=10.8, 8.5 Hz, 1H), 6.99 (dt, J=7.2, 2.0 Hz, 1H), 6.89 (s, 1H), 2.05 (s, 3H), 1.97-1.88 (m, 1H), 0.87-0.74 (m, 4H).

Step B: N-[5-[2-[[(1R,5S)-3-oxa-9-azabicyclo[3.3.1]nonan-7-yl]oxy]-5-prop-1-ynyl-phenyl]pyrazolo[1,5-a]pyridin-2-yl]cyclopropanecarboxamide. The title compound was prepared using the methods described for Example 165. MS (ESI): mass calcd. for C₂₇H₂₈N₄O₃, 456.2. m/z found, 457.3 [M+H]⁺. ¹H NMR (400 MHz, DMSO-d₆) δ 11.03 (s, 1H), 8.45 (dt, J=7.2, 1.0 Hz, 1H), 7.88 (dd, J=2.0, 0.9 Hz, 1H), 7.43 (d, J=2.2 Hz, 1H), 7.37 (dd, J=8.5, 2.2 Hz, 1H), 7.12-7.03 (m, 2H), 6.79 (s, 1H), 4.65 (p, J=6.4 Hz, 1H), 3.62-3.41 (m, 4H), 2.92 (d, J=8.1 Hz, 2H), 2.35-2.18 (m, 3H), 2.02 (s, 3H), 1.97-1.83 (m, 1H), 1.62 (ddd, J=13.7, 6.6, 2.5 Hz, 2H), 0.88-0.75 (m, 4H).

### Example 480: N-[5-[2-[[(1R,5S)-3-oxa-9-azabicyclo[3.3.1]nonan-7-yl]oxy]-5-propanoyl-phenyl]pyrazolo[1,5-a]pyridin-2-yl]cyclopropanecarboxamide

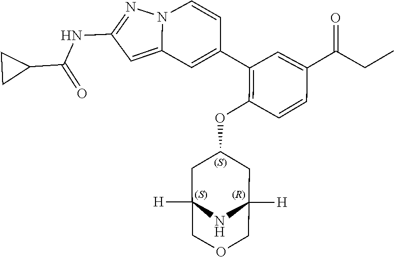

The title compound was isolated as a by-product from Example 479. MS (ESI): mass calcd. for C₂₇H₃₀N₄O₄, 474.2. m/z found, 457.2 [M+H]⁺. ¹H NMR (400 MHz, DMSO-d₆) δ 11.04 (s, 1H), 8.50 (dt, J=7.2, 1.0 Hz, 1H), 8.04-7.93 (m, 3H), 7.22 (d, J=8.8 Hz, 1H), 7.14 (dd, J=7.2, 2.0 Hz, 1H), 6.82 (s, 1H), 4.80 (p, J=6.2 Hz, 1H), 3.63-3.44 (m, 4H), 3.18 (d, J=5.2 Hz, 1H), 3.06 (q, J=7.2 Hz, 2H), 2.95 (d, J=8.3 Hz, 2H), 2.39-2.22 (m, 3H), 1.98-1.87 (m, 1H), 1.68 (ddd, J=13.8, 6.2, 2.4 Hz, 2H), 1.10 (t, J=7.2 Hz, 3H), 0.89-0.73 (m, 4H).

### Example 481: N-[5-[5-(difluoromethyl)-2-[[(1R,5S)-3-oxa-9-azabicyclo[3.3.1]nonan-7-yl]oxy]phenyl]pyrazolo[1,5-a]pyridin-2-yl]cyclopropanecarboxamide

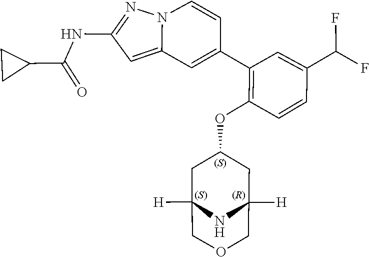

The title compound was prepared using methods analogous to those described in Example 479. MS (ESI): mass calcd. for C₂₅H₂₆F₂N₄O₃, 468.2. m/z found, 469.2 [M+H]⁺. ¹H NMR (400 MHz, DMSO-d₆) δ 11.04 (s, 1H), 8.49 (dd, J=7.2, 1.0 Hz, 1H), 7.92 (dd, J=2.0, 0.9 Hz, 1H), 7.68-7.61 (m, 1H), 7.61-7.52 (m, 1H), 7.24 (d, J=8.7 Hz, 1H), 7.10 (dd, J=7.3, 2.0 Hz, 1H), 7.01 (t, J=56.1 Hz, 1H), 6.82 (s, 1H), 4.73 (p, J=6.2 Hz, 1H), 3.63-3.42 (m, 4H), 2.94 (d, J=8.2 Hz, 2H), 2.31 (m, J=14.2, 8.6, 6.2 Hz, 3H), 1.99-1.86 (m, 1H), 1.66 (ddd, J=13.8, 6.5, 2.5 Hz, 2H), 0.90-0.74 (m, 4H).

### Example 482: N-[5-[5-(difluoromethyl)-2-[[(1R,5S)-9-methyl-3-oxa-9-azabicyclo[3.3.1]nonan-7-yl]oxy]phenyl]pyrazolo[1,5-a]pyridin-2-yl]cyclopropanecarboxamide

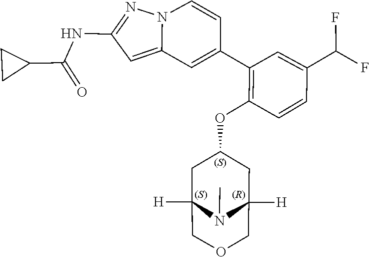

The title compound was prepared by treating N-[5-[5-(difluoromethyl)-2-[[(1R,5S)-3-oxa-9-azabicyclo[3.3.1]nonan-7-yl]oxy]phenyl]pyrazolo[1,5-a]pyridin-2-yl]cyclopropanecarboxamide (Example 481) with methylation conditions from Example 164. MS (ESI): mass calcd. for C₂₆H₂₈F₂N₄O₃, 482.2. m/z found, 483.3 [M+H]⁺. ¹H NMR (400 MHz, DMSO-d₆) δ 11.03 (s, 1H), 8.50-8.41 (m, 1H), 8.10 (dd, J=2.0, 0.9 Hz, 1H), 7.67 (dd, J=2.3, 1.2 Hz, 1H), 7.59-7.51 (m, 1H), 7.19 (dd, J=7.3, 2.0 Hz, 1H), 7.14 (s, 1H), 7.13-6.86 (m, 1H), 6.80 (s, 1H), 4.79-4.59 (m, 1H), 3.78 (dd, J=10.8, 2.4 Hz, 2H), 3.48 (d, J=10.7 Hz, 2H), 2.67-2.58 (m, 2H), 2.47-2.33 (m, 5H), 1.97-1.86 (m, 1H), 1.68-1.57 (m, 2H), 0.82 (tt, J=7.8, 3.0 Hz, 4H). ¹⁹F NMR (376 MHz, DMSO-d₆) δ −106.99.

### Example 483: 3-[2-[(2,6-dimethylpyrimidin-4-yl)amino]pyrazolo[1,5-a]pyridin-5-yl]-4-(4-hydroxycyclohexoxy)benzonitrile

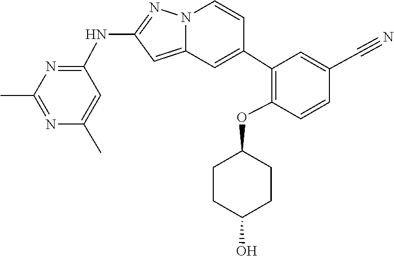 [SMILES: Cc1cc(Nc2cc3cc(-c4cc(C#N)ccc4O[C@H]4CC[C@H](O)CC4)ccn3n2)nc(C)n1]

The title compound was prepared using methods analogous to those described in Example 198. MS (ESI): mass calcd. for C₂₆H₂₆N₆O₂, 454.2. m/z found, 455.2 [M+H]⁺. ¹H NMR (500 MHz, DMSO-d₆) δ 10.17 (s, 1H), 8.57-8.51 (m, 1H), 7.87 (d, J=2.2 Hz, 1H), 7.83 (s, 1H), 7.69 (dd, J=2.1, 0.9 Hz, 1H), 7.37 (d, J=8.9 Hz, 1H), 7.02 (s, 1H), 6.94 (dd, J=7.2, 2.0 Hz, 1H), 6.81 (s, 1H), 4.64-4.57 (m, 1H), 4.57-4.52 (m, 1H), 3.53 (s, 1H), 2.45 (s, 3H), 2.30 (s, 3H), 2.05-1.96 (m, 2H), 1.79-1.70 (m, 2H), 1.51-1.40 (m, 2H), 1.40-1.30 (m, 2H).

### Example 484: 3-[2-[(2,6-dimethylpyrimidin-4-yl)amino]pyrazolo[1,5-a]pyridin-5-yl]-4-(2-hydroxy-2-methyl-propoxy)benzonitrile

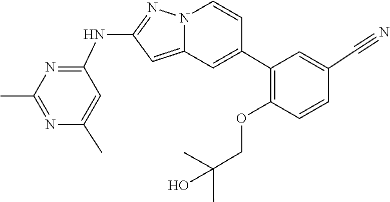 [SMILES: Cc1cc(Nc2cc3cc(-c4cc(C#N)ccc4OCC(C)(C)O)ccn3n2)nc(C)n1]

The title compound was prepared using methods analogous to those described in Example 198. MS (ESI): mass calcd. for C₂₄H₂₄N₆O₂, 428.2. m/z found, 429.2 [M+H]⁺. ¹H NMR (500 MHz, CDCl₃) δ 8.38 (dt, J=7.1, 0.9 Hz, 1H), 7.71-7.64 (m, 2H), 7.58 (s, 1H), 7.48 (dd, J=2.0, 0.9 Hz, 1H), 7.08-7.02 (m, 2H), 6.85 (dd, J=7.2, 1.9 Hz, 1H), 6.55 (s, 1H), 3.91 (s, 2H), 2.59 (s, 3H), 2.44 (s, 3H), 1.97 (s, 1H), 1.28 (s, 6H).

### Example 485: N-[5-[6-chloro-3-(1-methylazetidin-3-yl)oxy-pyridazin-4-yl]pyrazolo[1,5-a]pyridin-2-yl]cyclopropanecarboxamide

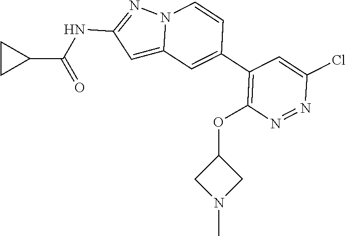 [SMILES: CN1CC(Oc2nnc(Cl)cc2-c2ccn3nc(NC(=O)C4CC4)cc3c2)C1]

Step A: N-(5-(3,6-dichloropyridazin-4-yl)pyrazolo[1,5-a]pyridin-2-yl)cyclopropanecarboxamide. To a mixture of Intermediate 3 (1.72 g, 5.27 mmol, 1.20 equiv), 4-bromo-3,6-dichloropyridazine (1.00 g, 4.39 mmol, 1.00 equiv), K₂CO₃ (2.43 g, 17.6 mmol, 4.00 equiv), 1,4-dioxane (15 mL) and water (3 mL) was sparged with nitrogen for 1 min and treated with PdCl₂(dtbpf) (286 mg, 0.439 mmol. 10 mol %). The mixture was stirred at 80° C. for 2 hours and then concentrated to dryness in vacuo. The crude material was purified by silica gel chromatography (0-100% EtOAc in petroleum ether) to give the title compound (800 mg, 2.30 mmol, 52% yield) as a yellow solid.

Step B: N-[5-[6-chloro-3-(1-methylazetidin-3-yl)oxy-pyridazin-4-yl]pyrazolo[1,5-a]pyridin-2-yl]cyclopropanecarboxamide. To a suspension of 1-methylazetidin-3-ol hydrochloride (160 mg, 1.29 mmol, 1.50 equiv) in DMA (3 mL) was added NaH (60 wt %, 172 mg, 4.31 mmol, 5.00 equiv). The resulting mixture was stirred for 10 minutes and then treated with a solution of N-(5-(3,6-dichloropyridazin-4-yl)pyrazolo[1,5-a]pyridin-2-yl)cyclopropanecarboxamide (300 mg, 0.86 mmol, 1.00 equiv) in DMA (2 mL). The resulting mixture was stirred at 80° C. for 2 h and then quenched with water (50 mL) and extracted with EtOAc (2×100 mL). The combined organics were dried with Na₂SO₄, filtered and concentrated in vacuo. The crude product was purified by silica gel chromatography (eluent: petroleum ether:ethyl acetate=1:0 to 0:1) to afford the impure product, the product was purified by preparative HPLC using a Waters Xbridge Prep OBD C18 150 mm×30 mm×5 μm column (eluent: 20% to 50% (v/v) CH₃CN and H₂O with 10 mM NH₄HCO₃) to afford the product. The product was suspended in water (10 mL), the mixture frozen using dry ice/ethanol, and then lyophilized to dryness to afford the title compound (27.4 mg, 0.07 mmol 8% yield) as a yellow solid. MS (ESI): mass calcd. for C₁₉H₁₉ClN₆O₂, 398.1. m/z found, 399.2 [M+H]⁺. ¹H NMR (400 MHz, DMSO-d₆) δ 11.16 (s, 1H), 8.66 (d, J=7.2 Hz, 1H), 8.06 (s, 2H), 7.23-7.17 (m, 1H), 6.98 (s, 1H), 5.35-5.25 (m, 1H), 3.77-3.66 (m, 2H), 3.14-3.07 (m, 2H), 2.29 (s, 3H), 1.99-1.89 (m, 1H), 0.91-0.74 (m, 4H).

### Example 486: 5-[6-methyl-3-[[(1S,5R)-3-oxa-9-azabicyclo[3.3.1]nonan-7-yl]oxy]pyridazin-4-yl]-N-pyrazin-2-yl-pyrazolo[1,5-a]pyridin-2-amine

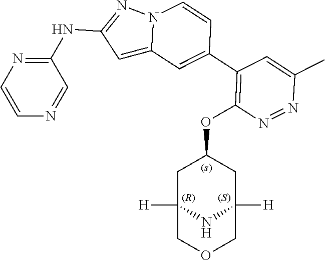

The title compound was prepared using methods analogous to those described in Example 488. MS (ESI): mass calcd. for C₂₃H₂₄N₈O₂, 444.2. m/z found, 445.2 [M+H]⁺. ¹H NMR (400 MHz, DMSO-d₆) δ 10.28 (s, 1H), 8.63 (d, J=1.5 Hz, 1H), 8.60 (d, J=7.2 Hz, 1H), 8.30-8.17 (m, 2H), 8.01 (d, J=2.8 Hz, 1H), 7.71 (s, 1H), 7.23 (dd, J=7.3, 2.0 Hz, 1H), 6.90 (s, 1H), 5.50 (p, J=5.8 Hz, 1H), 3.69-3.51 (m, 4H), 2.95 (d, J=7.8 Hz, 2H), 2.57 (s, 3H), 2.45-2.28 (m, 2H), 1.85 (ddd, J=14.3, 5.4, 2.2 Hz, 2H).

### Example 487: [4-[4-[2-[(2,6-dimethylpyrimidin-4-yl)amino]pyrazolo[1,5-a]pyridin-5-yl]-6-methyl-pyridazin-3-yl]oxycyclohexyl] acetate

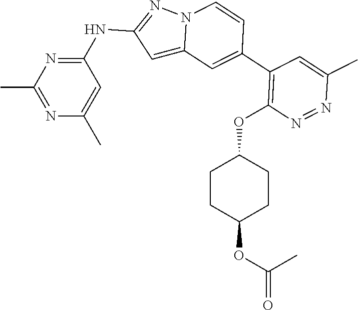

The title compound was isolated as a side product from Step E in Example 488. MS (ESI): mass calcd. for C₂₆H₂₉N₇O₃, 487.2. m/z found, 488.3 [M+H]⁺. ¹H NMR (400 MHz, DMSO-d₆) δ 10.22 (s, 1H), 8.63 (dt, J=7.2, 0.9 Hz, 1H), 7.94 (dd, J=2.0, 0.9 Hz, 1H), 7.71 (s, 1H), 7.09 (dd, J=7.3, 2.0 Hz, 1H), 7.00 (s, 1H), 6.91 (s, 1H), 5.41-5.29 (m, 1H), 4.83-4.72 (m, 1H), 2.57 (s, 3H), 2.46 (s, 3H), 2.30 (s, 3H), 2.19-2.06 (m, 2H), 2.01 (s, 3H), 1.96-1.86 (m, 2H), 1.77-1.64 (m, 2H), 1.64-1.52 (m, 2H).

### Example 488: 4-[4-[2-[(2,6-dimethylpyrimidin-4-yl)amino]pyrazolo[1,5-a]pyridin-5-yl]-6-methyl-pyridazin-3-yl]oxycyclohexanol

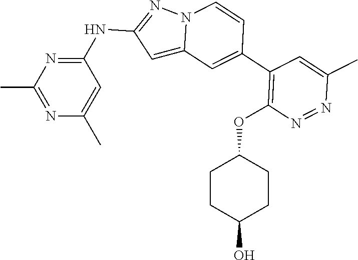

Step A: tert-butyl (5-(6-methyl-3-oxo-2,3-dihydropyridazin-4-yl)pyrazolo[1,5-a]pyridin-2-yl)carbamate. To flask charged with tert-butyl (5-(4,4,5,5-tetramethyl-1,3,2-dioxaborolan-2-yl)pyrazolo[1,5-a]pyridin-2-yl)carbamate (4.20 g, 11.6 mmol, 1.10 equiv), 4-bromo-6-methylpyridazin-3(2H)-one (2.00 g, 10.6 mmol, 1.00 equiv), PdCl₂(dppf) (229 mg, 0.31 mmol, 3 mol %) was evacuated and backfilled with N₂ (2×) then 1,4-dioxane (30 mL) and tribasic potassium phosphate (0.5 M, 30 mL, 15 mmol, 1.4 equiv) was added and the resulting mixture was stirred at 60° C. After 2 h, the reaction was cooled to 23° C. and the mixture was poured in to 150 mL of stirring water resulting in precipitate formation. The solids were collected by filtration and dried in vacuo overnight to provide the title compound (3.87 g, 11.4 mmol, 107%) which was used as in the following reaction.

Step B: 4-(2-aminopyrazolo[1,5-a]pyridin-5-yl)-6-methylpyridazin-3(2H)-one. To a solution of tert-butyl (5-(6-methyl-3-oxo-2,3-dihydropyridazin-4-yl)pyrazolo[1,5-a]pyridin-2-yl)carbamate (1.27 g, 3.73 mmol, 1.00 equiv) in DCM (17 mL) was added TFA (2.85 mL, 37.3 mmol, 10.0 equiv) at 23° C. After 1 h, the reaction mixture was concentrated to dryness and then was added sat. aq. NaHCO₃ (50 mL) resulting in precipitate formation. The solids were collected by filtration and dried in vacuo overnight to give (0.70 g, 2.90 mmol, 78% yield) as a tan solid which was used as in the following reaction

Step C: 4-(2-((2,6-dimethylpyrimidin-4-yl)amino)pyrazolo[1,5-a]pyridin-5-yl)-6-methylpyridazin-3(2H)-one. To a vial charged with 4-(2-aminopyrazolo[1,5-a]pyridin-5-yl)-6-methylpyridazin-3(2H)-one (680 mg, 2.82 mmol, 1.00 equiv), Cs₂CO₃ (3.00 g, 9.20 mmol, 3.27 equiv), tBuBrettPhos Pd G3 (128 mg, 0.15 mmol, 5 mol %) was evacuated and backfilled with Argon (2×) and then was added 1,4-dioxane (7 mL) followed by 4-chloro-2,6-dimethylpyrimidine (590 mg, 4.14 mmol, 1.50 equiv) as a solution in 1,4-dioxane (3 mL). The resulting mixture was stirred at 90° C. for 4 hours and then cooled to 23° C. The reaction mixture was diluted with EtOAc and the solids collected by filtration. The solids were washed with water and then dried in vacuo overnight to give the title compound (710 mg, 2.04 mmol, 73% yield) as brown solid which was used as is in the following reaction.

Step D: 5-(3-chloro-6-methylpyridazin-4-yl)-N-(2,6-dimethylpyrimidin-4-yl)pyrazolo[1,5-a]pyridin-2-amine. To vial containing 4-(2-((2,6-dimethylpyrimidin-4-yl)amino)pyrazolo[1,5-a]pyridin-5-yl)-6-methylpyridazin-3(2H)-one (205 mg, 0.59 mmol, 1.00 equiv), phosphoryl chloride (0.55 mL, 5.9 mmol, 10.0 equiv) in MeCN (1.2 mL) was stirred at 90° C. After 1.5 h, additional phosphoryl chloride (0.27 mL, 2.95 mmol, 5.00 equiv) was added and stirred for 1 hr and then cooled to 23° C. The reaction mixture was then diluted with MeCN and treated with 1 N NaOH until the aqueous became basic which resulted in precipitate formation. The solids were collected by filtration and dried in vacuo overnight to give (224 mg, 0.61 mmol, 104% yield) as a dark brown solid which was used as is in the following reaction.

Step E: 4-[4-[2-[(2,6-dimethylpyrimidin-4-yl)amino]pyrazolo[1,5-a]pyridin-5-yl]-6-methyl-pyridazin-3-yl]oxycyclohexanol. To a vial charged with trans-1,4-cyclohexanediol (221 mg, 1.90 mmol, 4.00 equiv), 5-(3-chloro-6-methylpyridazin-4-yl)-N-(2,6-dimethylpyrimidin-4-yl)pyrazolo[1,5-a]pyridin-2-amine (173 mg, 0.47 mmol, 1.00 equiv) and DMA (4 mL) was added KO t Bu (318 mg, 2.84 mmol, 6.00 equiv) and the resulting mixture was stirred at 50° C. After 3 hrs, the mixture was concentrated and purified by silica gel chromatography (0-10% MeOH in DCM) to give the title compound (22 mg, 0.04 mmol, 8% yield). MS (ESI): mass calcd. for C₂₄H₂₇N₇O₂, 445.2. m/z found, 446.2 [M+H]⁺. ¹H NMR (400 MHz, DMSO-d₆) δ 10.22 (s, 1H), 8.62 (dt, J=7.2, 0.9 Hz, 1H), 7.93 (dd, J=2.0, 0.9 Hz, 1H), 7.69 (s, 1H), 7.07 (dd, J=7.2, 2.0 Hz, 1H), 7.00 (s, 1H), 6.90 (s, 1H), 5.33-5.20 (m, 1H), 4.57 (s, 1H), 3.66-3.49 (m, 1H), 2.57 (s, 3H), 2.46 (s, 3H), 2.30 (s, 3H), 2.13 (m, J=12.9, 3.9 Hz, 2H), 1.89-1.76 (m, 2H), 1.61-1.48 (m, 2H), 1.43-1.30 (m, 2H).

### Example 489: N-[5-[3-[[(3R)-1-methylpyrrolidin-3-yl]methoxy]-4-pyridyl]pyrazolo[1,5-a]pyridin-2-yl]cyclopropanecarboxamide

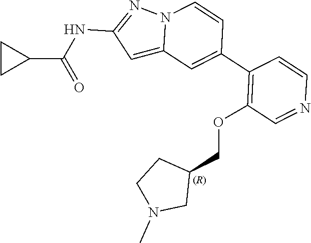

The title compound was prepared using methods analogous to those described in Example 136. MS (ESI): mass calcd. for C₂₂H₂₅N₅O₂, 391.2. m/z found, 392.2 [M+H]⁺. ¹H NMR (400 MHz, DMSO-d₆) δ 11.08 (s, 1H), 8.58 (d, J=7.1 Hz, 1H), 8.48 (s, 1H), 8.32-8.26 (m, 1H), 7.85 (d, J=2.0 Hz, 1H), 7.48 (d, J=4.8 Hz, 1H), 7.05 (dd, J=7.2, 2.0 Hz, 1H), 6.89 (s, 1H), 4.06 (d, J=6.3 Hz, 2H), 2.45-2.34 (m, 2H), 2.34-2.25 (m, 1H), 2.20 (s, 3H), 1.97-1.81 (m, 2H), 1.57-1.44 (m, 1H), 0.82 (tt, J=7.9, 3.0 Hz, 4H).

### Example 490: N-[5-[2-chloro-5-[[(3R)-1-methylpyrrolidin-3-yl]methoxy]-4-pyridyl]pyrazolo[1,5-a]pyridin-2-yl]cyclopropanecarboxamide

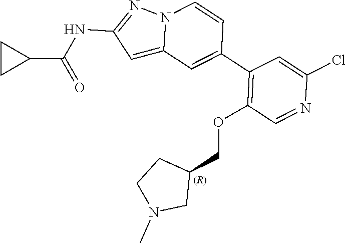

The title compound was prepared using methods analogous to those described for Intermediate 10. MS (ESI): mass calcd. for C₂₂H₂₄ClN₅O₂, 425.2. m/z found, 426.2 [M+H]⁺. ¹H NMR (400 MHz, DMSO-d₆) δ 11.10 (s, 1H), 8.58 (d, J=7.3 Hz, 1H), 8.28 (s, 1H), 7.88 (d, J=1.9 Hz, 1H), 7.59 (s, 1H), 7.05 (dd, J=7.2, 2.0 Hz, 1H), 6.90 (s, 1H), 4.05 (d, J=6.3 Hz, 2H), 2.43-2.31 (m, 2H), 2.31-2.24 (m, 1H), 2.19 (s, 3H), 1.97-1.79 (m, 2H), 1.56-1.42 (m, 1H), 0.89-0.75 (m, 4H).

### Example 491: N-[5-[2-chloro-5-(1-methylazetidin-3-yl)oxy-4-pyridyl]pyrazolo[1,5-a]pyridin-2-yl]cyclopropanecarboxamide

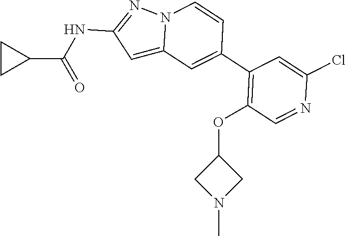 [SMILES: CN1CC(Oc2cnc(Cl)cc2-c2ccn3nc(NC(=O)C4CC4)cc3c2)C1]

The title compound was prepared using methods analogous to those described for Intermediate 10. MS (ESI): mass calcd. for C₂₀H₂₀ClN₅O₂, 397.1. m/z found, 398.1 [M+H]⁺. ¹H NMR (400 MHz, DMSO-d₆) δ 11.10 (s, 1H), 8.59 (dd, J=7.2, 1.0 Hz, 1H), 8.03 (s, 1H), 7.89 (dd, J=2.0, 0.9 Hz, 1H), 7.62 (s, 1H), 7.08 (dd, J=7.3, 2.0 Hz, 1H), 6.96-6.88 (m, 1H), 4.95 (p, J=5.5 Hz, 1H), 3.76-3.64 (m, 2H), 3.06-2.94 (m, 2H), 2.26 (s, 3H), 1.99-1.83 (m, 1H), 0.82 (m, J=7.8, 3.0 Hz, 4H).

### Example 492: N-[5-[2-chloro-5-[[(1S,5R)-3-oxa-9-azabicyclo[3.3.1]nonan-7-yl]oxy]-4-pyridyl]pyrazolo[1,5-a]pyridin-2-yl]cyclopropanecarboxamide

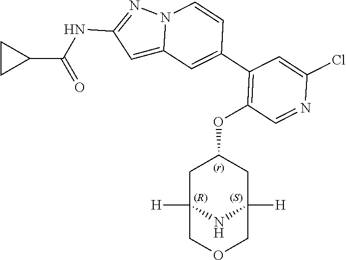

The title compound was prepared using methods analogous to those described for Intermediate 10, followed by a Boc-deprotection step similar to that described in Example 9, Step B. MS (ESI): mass calcd. for C₂₃H₂₄ClN₅O₃, 453.2. m/z found, 454.2 [M+H]⁺. ¹H NMR (400 MHz, DMSO-d₆) δ 11.09 (s, 1H), 8.58 (dt, J=7.3, 1.0 Hz, 1H), 8.29 (s, 1H), 7.88 (dd, J=2.1, 0.9 Hz, 1H), 7.60 (s, 1H), 7.07 (dd, J=7.3, 2.0 Hz, 1H), 6.91 (s, 1H), 5.41 (tt, J=10.9, 6.0 Hz, 1H), 3.70 (d, J=10.7 Hz, 2H), 3.61 (dt, J=10.9, 2.2 Hz, 2H), 2.92 (s, 2H), 2.26 (dd, J=12.3, 6.1 Hz, 2H), 1.98-1.87 (m, 1H), 1.69 (td, J=11.9, 4.8 Hz, 2H), 0.89-0.72 (m, 4H).

### Example 493: 1-[[6-(difluoromethyl)-4-[2-[(2,6-dimethylpyrimidin-4-yl)amino]pyrazolo[1,5-a]pyridin-5-yl]-3-pyridyl]oxy]-2-methyl-propan-2-ol

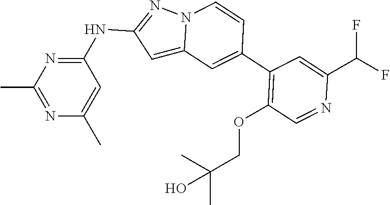 [SMILES: Cc1cc(Nc2cc3cc(-c4cc(C(F)F)ncc4OCC(C)(C)O)ccn3n2)nc(C)n1]

The title compound was prepared using methods analogous to those described in Example 329 Step D followed by the method in Example 52. MS (ESI): mass calcd. for C₂₃H₂₄F₂N₆O₂, 454.2. m/z found, 455.2 [M+H]⁺. ¹H NMR (400 MHz, DMSO-d₆) δ 10.22 (s, 1H), 8.62 (dt, J=7.2, 0.9 Hz, 1H), 8.59 (s, 1H), 7.95 (dd, J=2.0, 0.9 Hz, 1H), 7.78 (s, 1H), 7.15-7.07 (m, 1H), 7.03 (s, 1H), 6.96 (t, J=56.3 Hz, 1H), 6.87 (s, 1H), 4.70 (s, 1H), 4.03 (s, 2H), 2.46 (s, 3H), 2.30 (s, 3H), 1.17 (s, 6H). ¹⁹F NMR (376 MHz, DMSO-d₆) δ −113.22.

### Example 494: 3-[[4-[2-[(2,6-dimethylpyrimidin-4-yl)amino]pyrazolo[1,5-a]pyridin-5-yl]-6-(trifluoromethyl)-3-pyridyl]oxy]-2,2-dimethyl-propanenitrile

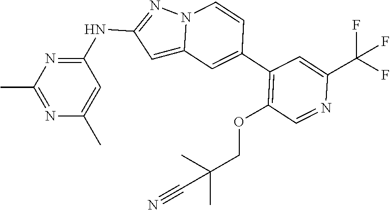 [SMILES: Cc1cc(Nc2cc3cc(-c4cc(C(F)(F)F)ncc4OCC(C)(C)C#N)ccn3n2)nc(C)n1]

The title compound was prepared using a method analogous to that described in Example 329 Step D followed by the method in Example 52. MS (ESI): mass calcd. for C₂₄H₂₂F₃N₇O, 481.2. m/z found, 482.2 [M+H]⁺. ¹H NMR (400 MHz, DMSO-d₆) δ 10.25 (s, 1H), 8.70 (s, 1H), 8.64 (dd, J=7.2, 1.0 Hz, 1H), 8.02 (s, 1H), 7.99 (dd, J=2.1, 0.9 Hz, 1H), 7.13 (dd, J=7.2, 2.0 Hz, 1H), 7.03 (s, 1H), 6.91 (s, 1H), 4.41 (s, 2H), 2.46 (s, 3H), 2.31 (s, 3H), 1.39 (s, 6H). ¹⁹F NMR (376 MHz, DMSO-d₆) δ −65.23.

### Example 495: 5-[2-chloro-5-[[(1S,4S,7R)-2-oxa-5-azabicyclo[2.2.1]heptan-7-yl]oxy]-4-pyridyl]-N-(2,6-dimethylpyrimidin-4-yl)pyrazolo[1,5-a]pyridin-2-amine

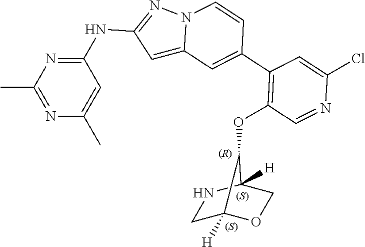

The title compound was prepared using a method analogous to that described in Example 329 Step D, the method in Example 52, followed by Boc-deprotection using the conditions described in Example 9, Step B. MS (ESI): mass calcd. for C₂₃H₂₂ClN₇O₂, 463.2. m/z found, 464.1 [M+H]⁺. ¹H NMR (500 MHz, DMSO-d₆) δ 10.22 (s, 1H), 8.62-8.58 (m, 1H), 8.51 (s, 1H), 7.86-7.82 (m, 1H), 7.62 (s, 1H), 7.04-6.97 (m, 2H), 6.88 (s, 1H), 4.95 (d, J=2.3 Hz, 1H), 4.35-4.31 (m, 1H), 3.87 (dd, J=7.3, 2.0 Hz, 1H), 3.74 (d, J=7.2 Hz, 1H), 3.49 (t, J=2.2 Hz, 1H), 3.10 (d, J=10.4 Hz, 1H), 2.81 (d, J=10.6 Hz, 1H), 2.46 (s, 3H), 2.30 (s, 3H).

### Example 496: N-(2,6-dimethylpyrimidin-4-yl)-5-[2-methoxy-5-[[(2S)-morpholin-2-yl]methoxy]-4-pyridyl]pyrazolo[1,5-a]pyridin-2-amine

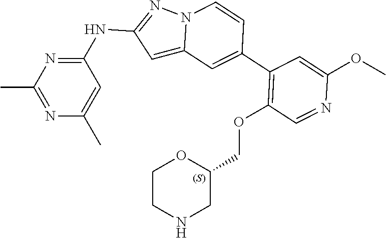

To a solution of tert-butyl 5-[2-chloro-5-[[(2S)-morpholin-2-yl]methoxy]-4-pyridyl]-N-(2,6-dimethylpyrimidin-4-yl)pyrazolo[1,5-a]pyridin-2-amine (Example 497, Step A, 50 mg, 0.0883 mmol) MeOH (0.537 mL, 0.791 g/mL, 13.25 mmol) and DMF (0.239 mL, 0.944 g/mL, 3.092 mmol) was added NaOMe (30% in MeOH) (0.0491 mL, 5.4 M, 0.265 mmol). The reaction was stirred at 100° C. for 1 h, then at 120° C. for 1 h, then at 140° C. for 4 h. The title compound was extracted using 20-80% ACN-H₂O with 20 mM of aq. NH₄OH as modifier to yield 6.6 mg (16%). MS (ESI): mass calcd. for C₂₄H₂₇N₇O₃, 461.2. m/z found, 462.2 [M+H]⁺. ¹H NMR (500 MHz, CD₃OD) δ 8.34 (dt, J=7.1, 0.9 Hz, 1H), 7.82 (s, 1H), 7.74 (dd, J=2.0, 0.9 Hz, 1H), 6.96-6.91 (m, 2H), 6.81 (d, J=0.5 Hz, 1H), 6.79 (s, 1H), 3.99-3.88 (m, 2H), 3.81 (s, 3H), 3.80-3.74 (m, 1H), 3.68 (ddd, J=11.5, 5.8, 3.5 Hz, 1H), 3.51 (ddd, J=11.5, 9.4, 4.8 Hz, 1H), 2.77 (dd, J=12.5, 2.4 Hz, 1H), 2.69-2.63 (m, 2H), 2.56 (dd, J=12.5, 10.5 Hz, 1H), 2.43 (s, 3H), 2.28 (d, J=0.7 Hz, 3H).

### Example 497: 5-[2-chloro-5-[[(2S)-morpholin-2-yl]methoxy]-4-pyridyl]-N-(2,6-dimethylpyrimidin-4-yl)pyrazolo[1,5-a]pyridin-2-amine

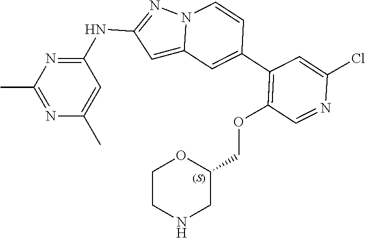

Step A: tert-butyl (S)-2-(((6-chloro-4-(2-((2,6-dimethylpyrimidin-4-yl)amino)pyrazolo[1,5-a]pyridin-5-yl)pyridin-3-yl)oxy)methyl)morpholine-4-carboxylate. To a vial was added Intermediate 26 (1700 mg, 4.64 mmol), tert-butyl (S)-2-((tosyloxy)methyl)morpholine-4-carboxylate (2066 mg, 5.56 mmol), cesium carbonate (3624 mg, 11.12 mmol) and DMA (37 mL, 4.64 mmol). The mixture was heated at 60° C. for 6 hr, then at 100° C. for 2 hr. The mixture was diluted with water/brine then extracted with ethyl acetate. After drying the organic layer with magnesium sulfate, the crude was purified via FCC (10-100% EtOAc/hexanes) to yield 975 mg (37%) of the title compound.

Step B: 5-[2-chloro-5-[[(2S)-morpholin-2-yl]methoxy]-4-pyridyl]-N-(2,6-dimethylpyrimidin-4-yl)pyrazolo[1,5-a]pyridin-2-amine. The title compound was prepared using the Boc-deprotection step described in Example 9, Step B. MS (ESI): mass calcd. for C₂₃H₂₄ClN₇O₂, 465.2. m/z found, 466.2 [M+H]⁺. ¹H NMR (500 MHz, DMSO-d₆) δ 10.15 (s, 1H), 8.54 (dt, J=7.2, 0.9 Hz, 1H), 8.23 (s, 1H), 7.92 (dd, J=2.0, 0.9 Hz, 1H), 7.56 (s, 1H), 7.03 (dd, J=7.2, 2.0 Hz, 1H), 6.92 (s, 1H), 6.85 (s, 1H), 4.10 (d, J=4.7 Hz, 2H), 3.75-3.68 (m, 1H), 3.62 (ddd, J=9.8, 7.1, 4.6 Hz, 1H), 3.40 (td, J=10.6, 3.6 Hz, 1H), 2.73 (dd, J=12.3, 2.4 Hz, 1H), 2.64-2.52 (m, 2H), 2.47 (d, J=10.0 Hz, 2H), 2.39 (s, 4H), 2.25-2.18 (m, 3H).

### Example 498: (2R,3S)-2-[[4-[2-[(2,6-dimethylpyrimidin-4-yl)amino]pyrazolo[1,5-a]pyridin-5-yl]-6-methoxy-3-pyridyl]oxymethyl]tetrahydrofuran-3-ol

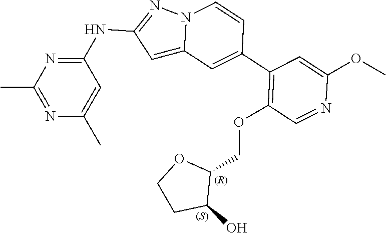

The title compound was prepared using a method analogous to that described in Example 329 Steps A and D. MS (ESI): mass calcd. for C₂₄H₂₆N₆O₄, 462.2. m/z found, 463.2 [M+H]⁺. ¹H NMR (500 MHz, DMSO-d₆) δ 10.19 (s, 1H), 8.62-8.51 (m, 1H), 8.02 (s, 1H), 7.87-7.78 (m, 1H), 7.02 (dd, J=7.2, 2.0 Hz, 2H), 6.92 (s, 1H), 6.86 (s, 1H), 5.03 (d, J=4.3 Hz, 1H), 4.17-4.11 (m, 1H), 4.10-4.03 (m, 2H), 3.87-3.74 (m, 6H), 2.46 (s, 3H), 2.30 (s, 3H), 1.95-1.86 (m, 1H), 1.75-1.61 (m, 1H).

### Example 499: (2R,3S)-2-[[4-[2-[(2,6-dimethylpyrimidin-4-yl)amino]pyrazolo[1,5-a]pyridin-5-yl]-6-(2,2,2-trifluoroethoxy)-3-pyridyl]oxymethyl]tetrahydrofuran-3-ol

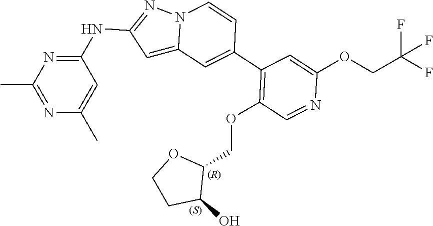

Step A: (2R,3S)-2-(((4-iodo-6-methoxypyridin-3-yl)oxy)methyl)tetrahydrofuran-3-ol. The title compound was prepared using a similar tosic acid displacement method described in Example 329, Step A.

Step B: (2R,3S)-2-(((4-(2-((2,6-dimethylpyrimidin-4-yl)amino)pyrazolo[1,5-a]pyridin-5-yl)-6-methoxypyridin-3-yl)oxy)methyl)tetrahydrofuran-3-ol. The title compound was prepared using a coupling method similar to that described in Example 329, Step D.

Step C: 4-(2-((2,6-dimethylpyrimidin-4-yl)amino)pyrazolo[1,5-a]pyridin-5-yl)-5-4(2R,3S)-3-hydroxytetrahydrofuran-2-yl)methoxy)pyridin-2(1H)-one. The title compound was prepared using an O-demethylation method similar to that described in Example 329, Step B.

Step D: (2R,3S)-2-[[4-[2-[(2,6-dimethylpyrimidin-4-yl)amino]pyrazolo[1,5-a]pyridin-5-yl]-6-(2,2,2-trifluoroethoxy)-3-pyridyl]oxymethyl]tetrahydrofuran-3-ol. To a vial was added 4-(2-((2,6-dimethylpyrimidin-4-yl)amino)pyrazolo[1,5-a]pyridin-5-yl)-5-(((2R,3S)-3-hydroxytetrahydrofuran-2-yl)methoxy)pyridin-2(1H)-one (100 mg, 0.22 mmol), cesium carbonate (218 mg, 0.67 mmol), and DMA (2.2 mL, 0.1 M). Stirred mixture at pre-heated 100° C. block for 5 min. Added 2-iodo-1,1,1-trifluoroethane (0.067 mL, 0.67 mmol) and stirred for 40 min. Filtered through Celite pad, then purified via Basic ISCO (Gemini 5 uM NX-C18 110 angstrom LC column 150×21.2 mm; 10-100% CH₃CN/20 mM aq NH₃ over 15 min; 40 mL/min). The fractions containing product were lyophilized to yield the title compound (26 mg, 22%). MS (ESI): mass calcd. for C₂₅H₂₅F₃N₆O₄, 530.2. m/z found, 531.2 [M+H]⁺. ¹H NMR (500 MHz, DMSO-d₆) δ 10.20 (s, 1H), 8.59 (d, J=7.2 Hz, 1H), 8.06 (s, 1H), 7.90-7.86 (m, 1H), 7.11 (s, 1H), 7.05 (dd, J=7.2, 2.0 Hz, 1H), 7.01 (s, 1H), 6.87 (s, 1H), 5.11-5.02 (m, 1H), 4.98 (q, J=9.1 Hz, 2H), 4.18-4.09 (m, 3H), 3.86-3.77 (m, 3H), 2.46 (s, 3H), 2.30 (s, 3H), 1.97-1.88 (m, 1H), 1.74-1.64 (m, 1H).

### Example 500: 2-[[4-[2-[(2,6-dimethylpyrimidin-4-yl)amino]pyrazolo[1,5-a]pyridin-5-yl]-5-[[(2R,3S)-3-hydroxytetrahydrofuran-2-yl]methoxy]-2-pyridyl]oxy]acetonitrile

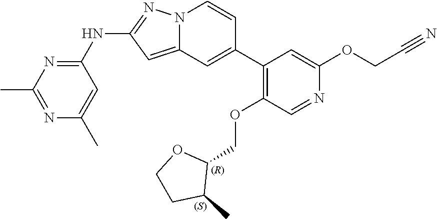

The title compound was prepared using methods analogous to those described in Example 499 and using bromoacetonitrile instead of 2-iodo-1,1,1-trifluoroethane in Step D. MS (ESI): mass calcd. for C₂₅H₂₅N₇O₄, 487.2. m/z found, 488.2 [M+H]⁺. ¹H NMR (500 MHz, DMSO-d₆) δ 10.20 (s, 1H), 8.63-8.56 (m, 1H), 8.10 (s, 1H), 7.89-7.86 (m, 1H), 7.10 (s, 1H), 7.04 (dd, J=7.2, 2.0 Hz, 1H), 7.01 (s, 1H), 6.88 (s, 1H), 5.20 (s, 2H), 5.05 (s, 1H), 4.18-4.10 (m, 3H), 3.88-3.75 (m, 3H), 2.46 (s, 3H), 2.30 (s, 3H), 1.96-1.87 (m, 1H), 1.74-1.66 (m, 1H).

### Example 501: N-(2,6-dimethylpyrimidin-4-yl)-5-[2-(methylamino)-5-[[(2S)-morpholin-2-yl]methoxy]-4-pyridyl]pyrazolo[1,5-a]pyridin-2-amine

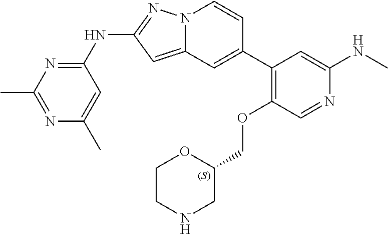

A solid mixture of tert-butyl (S)-2-(((6-chloro-4-(2-((2,6-dimethylpyrimidin-4-yl)amino)pyrazolo[1,5-a]pyridin-5-yl)pyridin-3-yl)oxy)methyl)morpholine-4-carboxylate (Example 497, Step A, 100 mg, 0.177 mmol) Cs₂CO₃ (115 mg, 0.353 mmol) and JosiPhos pre-catalyst gen. 3 (16.3 mg, 0.0177 mmol) was evacuated and back-filled with N₂ 3×. Then, toluene (0.488 mL, 0.867 g/mL, 4.59 mmol) and methylamine (0.883 mmol) was added. The reaction mixture was heated to 90° C. overnight. After 5 h, to the crude was added 30 mL of EtOAc and then washed with H₂O 2×10 mL. The organics were collected and concentrated down. The crude was purified via FCC using 10% MeOH/EtOAc. To this intermediate was performed a Boc-deprotection step similar to that described in Example 9, Step B to yield 8.5 mg (21%) of the title compound. MS (ESI): mass calcd. for C₂₄H₂₈N₈O₂, 460.2. m/z found, 461.2 [M+H]⁺. ¹H NMR (400 MHz, CD₃OD) δ 8.37-8.31 (m, 1H), 7.72-7.66 (m, 2H), 6.96-6.88 (m, 2H), 6.77 (s, 1H), 6.51 (s, 1H), 3.86-3.69 (m, 3H), 3.63 (dd, J=9.8, 6.1 Hz, 1H), 3.48 (ddd, J=11.5, 8.6, 5.6 Hz, 1H), 2.78 (s, 3H), 2.77-2.70 (m, 1H), 2.70-2.58 (m, 2H), 2.52 (dd, J=12.6, 10.5 Hz, 1H), 2.43 (s, 3H), 2.29 (s, 3H).

### Example 502: 5-[2-(difluoromethoxy)-5-[[(2S)-morpholin-2-yl]methoxy]-4-pyridyl]-N-(2,6-dimethylpyrimidin-4-yl)pyrazolo[1,5-a]pyridin-2-amine

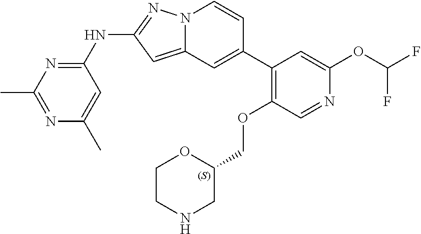

Step A: 5-(2-(difluoromethoxy)-5-fluoropyridin-4-yl)-N-(2,6-dimethylpyrimidin-4-yl)pyrazolo[1,5-a]pyridin-2-amine. To a solution of 4-bromo-2-(difluoromethoxy)-5-fluoropyridine (200 mg, 0.826 mmol), Intermediate 22 (362 mg, 0.992 mmol), Pd(dppf)Cl₂ (68.2 mg, 0.826 mmol), aq. K₃PO₄ (0.5 M, 2.8 mL) and 1,4-dioxane (2.8 mL) was purged with N₂ (evacuated and back-filled with N₂×3). The resulting mixture was heated at 95° C. for 1 hour. The mixture was then cooled to 23° C. and extracted 2×10% IPA/DCM/water. The organics were combined, dried (MgSO₄) and concentrated. The residue was purified by FCC (SiO₂, 100% EtOAc) to afford desired product as a yellow oil (180 mg, 70% pure, 38% yield). MS (ESI): mass calcd. for C₁₉H₁₅F₃N₆O, 400.13. m/z found, 401.1 [M+H]⁺.

Step B: tert-butyl (S)-2-(((6-(difluoromethoxy)-4-(2-((2,6-dimethylpyrimidin-4-yl)amino)pyrazolo[1,5-a]pyridin-5-yl)pyridin-3-yl)oxy)methyl)morpholine-4-carboxylate. To a small vial containing 5-(2-(difluoromethoxy)-5-fluoropyridin-4-yl)-N-(2,6-dimethylpyrimidin-4-yl)pyrazolo[1,5-a]pyridin-2-amine (180 mg, 0.45 mol) and tert-butyl (S)-2-(hydroxymethyl)morpholine-4-carboxylate (293 mg, 1.35 mmol) was added DMF (1.7 mL) followed by sodium hydride (60 wt % dispersion in mineral oil, 53.9 mg, 1.35 mmol) portion-wisely while stirring at rt. The suspension was heated at 90° C. for 16 h, then quenched with water. The crude was purified via prep. HPLC using 20-80% ACN-H₂O with 20 mM of NH₄OH as modifier and the product was lyophilized to give the title compound (268 mg, 100%) which was used in the next step with some impurities remaining.

Step C: 5-[2-(difluoromethoxy)-5-[[(2S)-morpholin-2-yl]methoxy]-4-pyridyl]-N-(2,6-dimethylpyrimidin-4-yl)pyrazolo[1,5-a]pyridin-2-amine. The title compound was prepared using methods analogous to those described in Example 9 Step B. MS (ESI): mass calcd. for C₂₄H₂₅F₂N₇O₃, 497.2. m/z found, 498.2 [M+H]⁺. ¹H NMR (400 MHz, DMSO-d₆) δ 10.16 (s, 1H), 8.54 (dd, J=7.2, 0.9 Hz, 1H), 8.05 (s, 1H), 7.92 (dd, J=2.0, 0.9 Hz, 1H), 7.58 (t, J=73.2 Hz, 1H), 7.18 (s, 1H), 7.02 (dd, J=7.2, 2.0 Hz, 1H), 6.92 (s, 1H), 6.85 (s, 1H), 4.05 (d, J=4.8 Hz, 2H), 3.71 (d, J=10.9 Hz, 1H), 3.62 (t, J=3.9 Hz, 1H), 3.40 (td, J=10.6, 3.9 Hz, 1H), 2.73 (d, J=12.1 Hz, 1H), 2.58 (dd, J=10.1, 3.2 Hz, 2H), 2.48 (s, 1H), 2.39 (s, 3H), 2.25-2.21 (m, 3H).

### Example 503: N-[5-[2-(difluoromethoxy)-5-[[(2S)-morpholin-2-yl]methoxy]-4-pyridyl]pyrazolo[1,5-a]pyridin-2-yl]cyclopropanecarboxamide

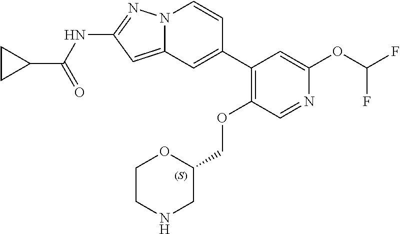

The title compound was prepared using methods analogous to those described in Example 502. MS (ESI): mass calcd. for C₂₂H₂₃F₂N₅O₄, 459.2. m/z found, 460.2 [M+H]⁺. ¹H NMR (400 MHz, DMSO-d₆) δ 11.04 (s, 1H), 8.53 (dt, J=7.3, 0.9 Hz, 1H), 8.04 (s, 1H), 7.90 (dd, J=2.0, 0.9 Hz, 1H), 7.58 (t, J=73.2 Hz, 1H), 7.17 (s, 1H), 7.05 (dd, J=7.3, 2.0 Hz, 1H), 6.90-6.73 (m, 1H), 4.04 (d, J=4.8 Hz, 2H), 3.72-3.64 (m, 1H), 3.60 (dtd, J=10.0, 4.8, 2.4 Hz, 1H), 3.38 (td, J=10.8, 3.1 Hz, 1H), 2.73 (dd, J=12.1, 2.4 Hz, 1H), 2.63-2.47 (m, 2H), 2.31 (s, 1H), 1.86 (dq, J=7.8, 4.9, 4.1 Hz, 1H), 0.76 (tt, J=7.9, 3.0 Hz, 4H).

### Example 504: N-[6-[5-(4-hydroxycyclohexoxy)-2-methyl-4-pyridyl]imidazo[1,2-a]pyridin-2-yl]cyclopropanecarboxamide

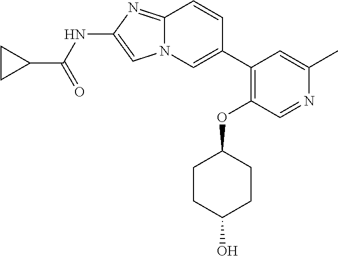 [SMILES: Cc1cc(-c2ccc3nc(NC(=O)C4CC4)cn3c2)c(O[C@H]2CC[C@H](O)CC2)cn1]

The title compound was prepared using the method analogous to that described in Example 158. MS (ESI): mass calcd. for C₂₃H₂₆N₄O₃, 406.2. m/z found, 407.2 [M+H]⁺. ¹H NMR (400 MHz, DMSO-d₆) δ 11.12 (s, 1H), 8.99 (t, J=1.4 Hz, 1H), 8.62 (s, 1H), 8.18 (s, 1H), 7.89 (s, 1H), 7.58 (d, J=1.3 Hz, 2H), 4.64 (tt, J=8.3, 3.7 Hz, 1H), 3.54 (tt, J=8.4, 3.8 Hz, 1H), 2.63 (s, 3H), 2.09-1.99 (m, 2H), 1.99-1.91 (m, 1H), 1.81-1.70 (m, 2H), 1.59-1.45 (m, 2H), 1.41-1.27 (m, 2H), 0.91-0.77 (m, 4H).

### Example 505: N-[6-[2-methyl-5-[[(1S,5R)-3-oxa-9-azabicyclo[3.3.1]nonan-7-yl]oxy]-4-pyridyl]imidazo[1,2-a]pyrazin-2-yl]cyclopropanecarboxamide

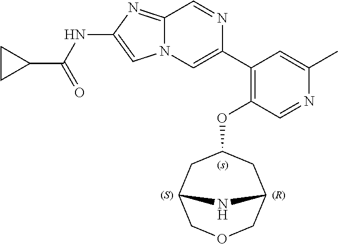

Step A. N-(6-bromoimidazo[1,2-a]pyrazin-2-yl)cyclopropanecarboxamide. To a 0° C. mixture of 6-bromoimidazo[1,2-a]pyrazin-2-amine (2000 mg, 9.388 mmol) and DCM (60.1 mL, 1.326 g/mL, 939 mmol) and TEA (3.92 mL, 0.728 g/mL, 28.164 mmol) was added cyclopropanecarbonyl chloride (2159 mg, 20.65 mmol). The reaction was allowed to reach r.t. in 2 h while stirring. The crude material was concentrated down under vacuo to a yellow solid. The yellow solid was stirred with 20 mL of 2 M NH₃ in MeOH for 2 h. To the crude reaction mixture was added H₂O while stirring, then filtered to afford an off-white solid (2.63 g, 100%). MS (ESI): mass calcd. for C₁₀H₉BrN₄O, 281.1. m/z found, 283.0 [M+H]⁺. ¹H NMR (400 MHz, DMSO-d₆) δ 11.33 (s, 1H), 8.92 (d, J=1.3 Hz, 1H), 8.74 (s, 1H), 8.26 (s, 1H), 1.96 (p, J=6.2 Hz, 1H), 0.84 (d, J=6.1 Hz, 4H).), 3.31 (s, 3H), 1.36 (s, 6H).

Step B. N-(6-(5-fluoro-2-methylpyridin-4-yl)imidazo[1,2-a]pyrazin-2-yl)cyclopropanecarboxamide. The title compound was prepared in the similar manner of Example 320 step A except N-(6-bromoimidazo[1,2-a]pyrazin-2-yl)cyclopropanecarboxamide from Step A was used instead of 6-bromoimidazo[1,2-a]pyrazin-2-amine (80 mg, 59%).

Step C. N-(6-(5-(((1R,5S,7s)-3-oxa-9-azabicyclo[3.3.1]nonan-7-yl)oxy)-2-methylpyridin-4-yl)imidazo[1,2-a]pyrazin-2-yl)cyclopropanecarboxamide. The title compound was prepared in the similar manner of Example 320 step C except sodium hydride was used instead of sodium tert-butoxide. Also, TFA was added to the crude reaction and stirred for 2 h at r.t. The title compound was purified via preparative HPLC to afford (71.8 mg, 64%). MS (ESI): mass calcd. for C₂₃H₂₆N₆O₃, 434.2. m/z found, 435.20 [M+H]⁺. ¹H NMR (500 MHz, CD₃OD) δ 9.91 (d, J=1.5 Hz, 1H), 8.90-8.86 (m, 1H), 8.16 (s, 1H), 8.12 (s, 1H), 7.99 (s, 1H), 4.92 (td, J=5.4, 4.5, 3.1 Hz, 1H), 3.95 (ddd, J=11.5, 2.8, 1.3 Hz, 2H), 3.85 (d, J=11.4 Hz, 2H), 3.05-2.93 (m, 2H), 2.52 (s, 3H), 2.44 (dtd, J=16.0, 6.3, 1.3 Hz, 2H), 2.16-2.04 (m, 2H), 1.91 (tt, J=8.6, 4.9 Hz, 1H), 1.00 (dt, J=4.7, 3.2 Hz, 2H), 0.96-0.87 (m, 2H).

### Example 506: 4-[[4-[2-[(2,6-dimethylpyrimidin-4-yl)amino]imidazo[1,2-a]pyrazin-6-yl]-6-methyl-3-pyridyl]oxy]cyclohexanol

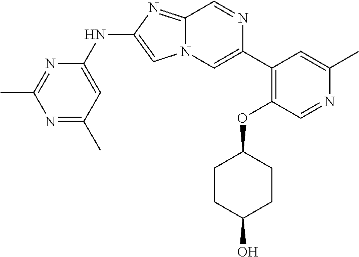

The title compound was prepared in the similar manner of Example 320 except (cis)-cyclohexane-1,4-diol was used instead of tert-butyl (1R,5S,7s)-7-hydroxy-3-oxa-9-azabicyclo[3.3.1]nonane-9-carboxylate and N-(2,6-dimethylpyrimidin-4-yl)-6-(5-fluoro-2-methylpyridin-4-yl)imidazo[1,2-a]pyrazin-2-amine was used instead of 6-(5-fluoro-2-methylpyridin-4-yl)-N-(5-methylpyrazin-2-yl)imidazo[1,2-a]pyrazin-2-amine (10.8 mg, 8.4%). MS (ESI): mass calcd. for C₂₄H₂₇N₇O₂, 445.2. m/z found, 446.1 [M+H]⁺. ¹H NMR (400 MHz, DMSO-d₆) δ 10.48 (s, 1H), 9.27 (s, 1H), 8.99 (s, 1H), 8.53 (s, 1H), 8.39 (s, 1H), 7.83 (s, 1H), 6.79 (s, 1H), 4.70-4.62 (m, 1H), 4.55-4.48 (m, 1H), 3.62-3.53 (m, 1H), 2.55-2.53 (m, 3H), 2.48-2.46 (m, 3H), 2.30 (s, 3H), 2.00-1.91 (m, 2H), 1.71-1.50 (m, 6H).

### Example 507: 4-[[4-[2-[(2,6-dimethylpyrimidin-4-yl)amino]imidazo[1,2-a]pyrazin-6-yl]-6-methyl-3-pyridyl]oxy]cyclohexanol

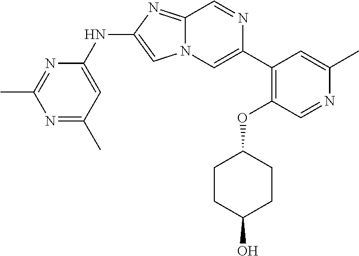

The title compound was prepared in the similar manner of Example 320 except (trans)-cyclohexane-1,4-diol was used instead of tert-butyl (1R,5S,7s)-7-hydroxy-3-oxa-9-azabicyclo[3.3.1]nonane-9-carboxylate and N-(2,6-dimethylpyrimidin-4-yl)-6-(5-fluoro-2-methylpyridin-4-yl)imidazo[1,2-a]pyrazin-2-amine was used instead of 6-(5-fluoro-2-methylpyridin-4-yl)-N-(5-methylpyrazin-2-yl)imidazo[1,2-a]pyrazin-2-amine (34.5 mg, 32%). MS (ESI): mass calcd. for C₂₄H₂₇N₇O₂, 445.2. m/z found, 446.1 [M+H]⁺. ¹H NMR (400 MHz, DMSO-d₆) δ 10.45 (s, 1H), 9.22 (s, 1H), 8.97 (s, 1H), 8.52 (s, 1H), 8.41 (s, 1H), 7.82 (s, 1H), 6.81 (s, 1H), 4.62-4.58 (m, 1H), 4.57-4.47 (m, 1H), 3.59-3.49 (m, 1H), 2.54 (s, 3H), 2.48 (s, 3H), 2.30 (s, 3H), 2.11-2.00 (m, 2H), 1.86-1.75 (m, 2H), 1.65-1.52 (m, 2H), 1.40-1.27 (m, 2H)

### Example 508: N-[6-[5-(2-cyano-2-methyl-propoxy)-2-methyl-4-pyridyl]imidazo[1,2-a]pyrazin-2-yl]cyclopropanecarboxamide

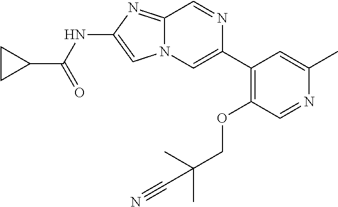 [SMILES: Cc1cc(-c2cn3cc(NC(=O)C4CC4)nc3cn2)c(OCC(C)(C)C#N)cn1]

The title compound was prepared in the similar manner of Example 505 except 3-hydroxy-2,2-dimethylpropanenitrile was used instead of tert-butyl (1R,5S,7s)-7-hydroxy-3-oxa-9-azabicyclo[3.3.1]nonane-9-carboxylate (38.2 mg, 30%). MS (ESI): mass calcd. for C₂₁H₂₂N₆O₂, 390.2. m/z found, 413.2 [M+H]⁺. ¹H NMR (500 MHz, CD₃OD) δ 9.24 (d, J=1.5 Hz, 1H), 8.84 (dd, J=1.5, 0.7 Hz, 1H), 8.20 (s, 1H), 8.17 (d, J=0.7 Hz, 1H), 7.87 (s, 1H), 4.16 (s, 2H), 2.46 (s, 3H), 1.81 (td, J=8.0, 4.1 Hz, 1H), 1.39 (s, 6H), 0.91 (dt, J=4.6, 3.1 Hz, 2H), 0.83 (dt, J=8.0, 3.2 Hz, 2H).

### Example 509: N-[5-[2-methyl-5-[[(3R)-1-methylpyrrolidin-3-yl]methoxy]-4-pyridyl]pyrazolo[1,5-a]pyridin-2-yl]cyclopropanecarboxamide

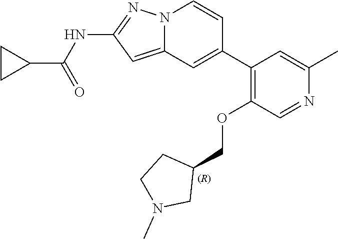

Step A: N-(5-(5-chloro-2-methylpyridin-4-yl)pyrazolo[1,5-a]pyridin-2-yl)cyclopropanecarboxamide. The title compound was prepared using the method described in Intermediate 6, except 4-bromo-5-choro-2-methylpyridine was used in place of 4-bromo-5-fluoro-2-methylpyridine.

Step B: N-[5-[2-methyl-5-[[(3R)-1-methylpyrrolidin-3-yl]methoxy]-4-pyridyl]pyrazolo[1,5-a]pyridin-2-yl]cyclopropanecarboxamide. To a 10 mL microwave vial was charged N-(5-(5-chloro-2-methylpyridin-4-yl)pyrazolo[1,5-a]pyridin-2-yl)cyclopropanecarboxamide (209 mg, 0.64 mmol), RockPhos Pd G3 (28 mg, 0.03 mmol), and RockPhos (18 mg, 0.04 mmol) as solids. The vial was sealed then evac and backfilled with argon (3×). To a separate 10 mL microwave vial was charged NaH (60% dispersion in mineral oil, 55 mg, 1.38 mmol). A solution of (R)-(1-methylpyrrolidin-3-yl)methanol (169 mg, 1.47 mmol) in toluene (2.18 mL, 0.3 M, 0.65 mmol) was then added in one portion. This mixture was allowed to stir for 15 min and was then was added to the above solids. The vial was then placed into a preheated 90° C. heating block for 16 hr. Diluted with 20 mL ethyl acetate and filtered though a pad of Celite. The filtrate was concentrated and purified via FCC (SiO₂, 0-10% (2M NH₃ in MeOH)/DCM to yield 35 mg of the title compound as a colorless solid (35 mg, 13%). MS (ESI): mass calcd. for C₂₃H₂₇N₅O₂, 405.2. m/z found, 406.20 [M+H]⁺. ¹H NMR (400 MHz, DMSO-d₆) δ 11.07 (s, 1H), 8.57 (d, J=7.2 Hz, 1H), 8.31 (s, 1H), 7.82 (d, J=1.9 Hz, 1H), 7.34 (s, 1H), 7.03 (dd, J=7.2, 2.0 Hz, 1H), 6.88 (s, 1H), 4.00 (d, J=6.2 Hz, 2H), 2.56-2.51 (m, 1H), 2.45 (s, 3H), 2.43-2.34 (m, 2H), 2.32-2.24 (m, 1H), 2.19 (s, 3H), 1.97-1.81 (m, 2H), 1.56-1.43 (m, 1H), 0.88-0.75 (m, 4H).

### Example 510: N-[5-[5-[(3S)-2,2-dimethylazetidin-3-yl]oxy-2-methyl-4-pyridyl]pyrazolo[1,5-a]pyridin-2-yl]cyclopropanecarboxamide

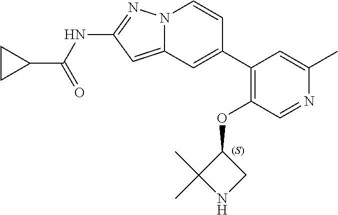

Step A: (S)—N-(5-(5-((1-benzhydryl-2,2-dimethylazetidin-3-yl)oxy)-2-methylpyridin-4-yl)pyrazolo[1,5-a]pyridin-2-yl)cyclopropanecarboxamide. The title compound was prepared using a similar method described in Example 136 and using (S)-1-benzhydryl-2,2-dimethylazetidin-3-ol instead of endo-7-hydroxy-3-oxa-9-azabicyclo[3.3.1]nonane hydrochloride.

Step B: N-[5-[5-[(3S)-2,2-dimethylazetidin-3-yl]oxy-2-methyl-4-pyridyl]pyrazolo[1,5-a]pyridin-2-yl]cyclopropanecarboxamide. To a mixture of 10% Pd/C (550 mg, dry) and MeOH (5 mL) was added a mixture of ammonium formate (746 mg, 11.8 mmol) and (S)—N-(5-(5-((1-benzhydryl-2,2-dimethylazetidin-3-yl)oxy)-2-methylpyridin-4-yl)pyrazolo[1,5-a]pyridin-2-yl)cyclopropanecarboxamide (550 mg, 0.986 mmol) in MeOH (5 mL) and THF (10 mL). The reaction was stirred at 50° C. for 16 h. The mixture was filtered and the organic phase concentrated to dryness under reduced pressure to give the crude product. The residue was purified by preparative HPLC using a Boston Prime C18 150×30 mm×5 μm column (eluent: 25% to 55% (v/v) CH₃CN and H₂O with 0.05% NH₃). The product was suspended in water (10 mL), the mixture frozen using dry ice/ethanol, and then lyophilized to dryness to afford the title compound as a white solid. MS (ESI): mass calcd. for C₂₂H₂₅N₅O₂, 391.2. m/z found, 392.2 [M+H]⁺. ¹H NMR (400 MHz, DMSO-d₆) δ 11.11 (s, 1H), 8.59 (d, J=7.2 Hz, 1H), 8.04 (s, 1H), 7.85 (s, 1H), 7.38 (s, 1H), 7.07 (dd, J=2.0, 7.2 Hz, 1H), 6.89 (s, 1H), 4.74 (t, J=6.4 Hz, 1H), 3.71-3.63 (m, 1H), 3.31-3.25 (m, 1H), 2.46 (s, 3H), 1.99-1.88 (m, 1H), 1.28 (s, 3H), 1.21 (s, 3H), 0.88-0.77 (m, 4H)

### Example 511: N-(1-isopropylpyrazol-4-yl)-5-[2-methyl-5-(1-methylazetidin-3-yl)oxy-4-pyridyl]pyrazolo[1,5-a]pyridin-2-amine

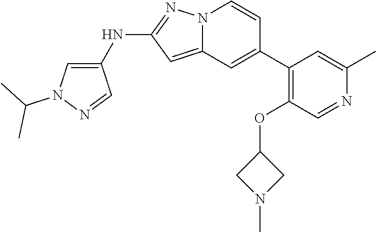 [SMILES: Cc1cc(-c2ccn3nc(Nc4cnn(C(C)C)c4)cc3c2)c(OC2CN(C)C2)cn1]

The title compound was prepared using methods analogous to those described in Example 162. MS (ESI): mass calcd. for C₂₃H₂₇N₇O, 417.2. m/z found, 418.3 [M+H]⁺. ¹H NMR (400 MHz, CDCl₃) δ 8.25 (d, J=7.2 Hz, 1H), 7.98 (s, 1H), 7.62 (s, 1H), 7.50 (s, 1H), 7.46 (d, J=1.2 Hz, 1H), 7.17 (s, 1H), 6.80 (dd, J=2.0, 7.2 Hz, 1H), 5.88 (s, 1H), 5.81 (s, 1H), 4.84-4.76 (m, 1H), 4.53-4.43 (m, 1H), 3.84-3.77 (m, 2H), 3.15-3.08 (m, 2H), 2.54 (s, 3H), 2.39 (s, 3H), 1.54 (d, J=6.4 Hz, 6H).

### Example 512: N-[5-[2-methyl-5-[[(2S)-1-methylazetidin-2-yl]methoxy]-4-pyridyl]pyrazolo[1,5-a]pyridin-2-yl]cyclopropanecarboxamide

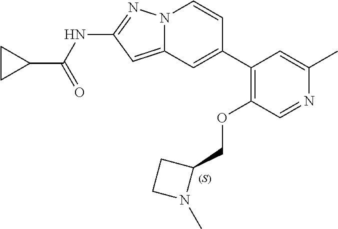

The title compound was prepared using methods analogous to those described in Example 136. MS (ESI): mass calcd. for C₂₂H₂₅N₅O₂, 391.2. m/z found, 392.2 [M+H]⁺. ¹H NMR (500 MHz, CDCl₃) δ 9.84-8.82 (m, 1H), 8.61-7.79 (m, 2H), 7.47 (d, J=22.7 Hz, 1H), 7.24-6.34 (m, 3H), 4.40-3.57 (m, 2H), 3.18 (t, J=30.9 Hz, 2H), 2.84-1.57 (m, 9H), 1.53-0.18 (m, 5H).

### Example 513: 5-[2-methyl-5-[[(2S,5R)-5-methylmorpholin-2-yl]methoxy]-4-pyridyl]-N-pyridazin-3-yl-pyrazolo[1,5-a]pyridin-2-amine

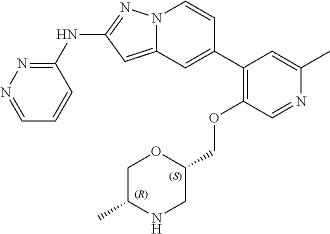

The title compound was prepared using methods analogous to those described in Example 116 Steps A and B. MS (ESI): mass calcd. for C₂₃H₂₅N₇O₂, 431.2. m/z found, 432.2 [M+H]⁺. ¹H NMR (400 MHz, CDCl₃) δ 8.60-8.48 (m, 2H), 8.48-8.36 (m, 2H), 7.75-7.62 (m, 2H), 6.97 (dd, J=7.2, 1.9 Hz, 1H), 6.67 (s, 1H), 5.32 (s, 3H), 4.25 (s, 2H), 4.08 (d, J=10.6 Hz, 1H), 3.77 (d, J=12.3 Hz, 1H), 3.52 (s, 2H), 3.32-3.22 (m, 1H), 3.15 (d, J=12.6 Hz, 1H), 2.65 (s, 3H), 1.38 (d, J=6.9 Hz, 3H).

### Example 514: 5-[2-methyl-5-[[(1S,5R)-8-methyl-8-azabicyclo[3.2.1]octan-3-yl]oxy]-4-pyridyl]-N-pyridazin-3-yl-pyrazolo[1,5-a]pyridin-2-amine

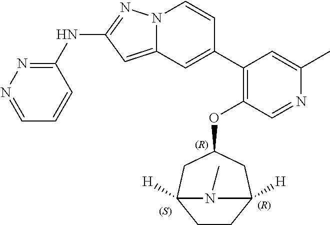

The title compound was prepared using methods analogous to those described in Example 116. MS (ESI): mass calcd. for C₂₅H₂₇N₇O, 441.2. m/z found, 442.2 [M+H]⁺. ¹H NMR (500 MHz, CDCl₃) δ 8.71 (q, J=6.0, 5.2 Hz, 1H), 8.43 (q, J=7.2, 6.6 Hz, 1H), 8.18 (t, J=6.8 Hz, 1H), 8.07-7.99 (m, 1H), 7.59 (t, J=6.8 Hz, 1H), 7.55-7.47 (m, 1H), 7.25 (d, J=13.6 Hz, OH), 6.88 (q, J=7.3, 6.3 Hz, 1H), 6.78 (t, J=6.8 Hz, 1H), 3.76 (s, 2H), 3.53-3.48 (m, 3H), 2.73-2.66 (m, 5H), 2.58 (t, J=6.9 Hz, 3H), 2.22 (dd, J=16.3, 5.3 Hz, 2H), 2.04 (t, J=12.5 Hz, 4H).

### Example 515: 5-[2-methyl-5-[[(2S)-4-methylmorpholin-2-yl]methoxy]-4-pyridyl]-N-pyrazin-2-yl-pyrazolo[1,5-a]pyridin-2-amine

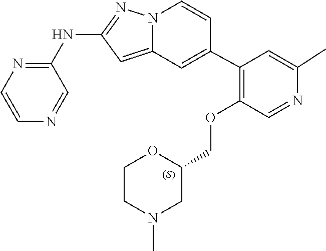

The title compound was prepared using methods analogous to those described in Example 116. MS (ESI): mass calcd. for C₂₃H₂₅N₇O₂, 431.2. m/z found, 432.2 [M+H]⁺. ¹H NMR (500 MHz, DMSO-d₆) δ 10.26 (s, 1H), 8.65 (d, J=1.5 Hz, 1H), 8.60 (d, J=7.1 Hz, 1H), 8.34 (s, 1H), 8.21 (dd, J=2.7, 1.5 Hz, 1H), 8.01 (d, J=2.7 Hz, 1H), 7.87 (dd, J=2.0, 0.9 Hz, 1H), 7.36 (s, 1H), 7.04 (dd, J=7.2, 2.0 Hz, 1H), 6.91 (d, J=0.8 Hz, 1H), 4.19-4.09 (m, 2H), 3.83-3.71 (m, 2H), 3.56-3.46 (m, 1H), 2.76-2.68 (m, 1H), 2.60-2.54 (m, 1H), 2.47 (s, 3H), 2.15 (s, 3H), 2.01-1.92 (m, 1H), 1.92-1.85 (m, 1H).

### Example 516: (*R)-5-[5-[(6,6-dimethylmorpholin-2-yl)methoxy]-2-methyl-4-pyridyl]-N-pyridazin-3-yl-pyrazolo[1,5-a]pyridin-2-amine

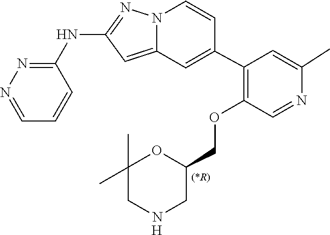

The title compound was prepared using methods analogous to those described in Example 116 Steps A and B. MS (ESI): mass calcd. for C₂₄H₂₇N₇O₂, 445.2. m/z found, 446.20 [M+H]⁺. ¹H NMR (500 MHz, CDCl₃) δ 9.80-9.76 (m, 1H), 8.80-8.75 (m, 1H), 8.41-8.36 (m, 1H), 8.27-8.18 (m, 2H), 7.72-7.67 (m, 1H), 7.42 (dd, J=9.1, 4.5 Hz, 1H), 7.19 (s, 1H), 7.00 (dd, J=7.2, 1.9 Hz, 1H), 6.73 (s, 1H), 4.10-4.00 (m, 2H), 4.03-3.94 (m, 1H), 3.82-3.76 (m, 1H), 3.57 (dd, J=11.3, 3.6 Hz, 1H), 3.47 (dd, J=11.3, 6.0 Hz, 1H), 2.99-2.92 (m, 1H), 2.87-2.82 (m, 1H), 2.70-2.49 (m, 3H), 1.32 (d, J=3.4 Hz, 3H), 1.19 (d, J=17.3 Hz, 3H).

### Example 517: N-[5-[5-(3-amino-3-methyl-cyclobutoxy)-2-methyl-4-pyridyl]pyrazolo[1,5-a]pyridin-2-yl]cyclopropanecarboxamide

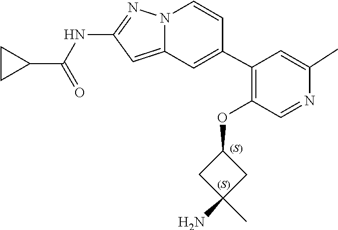

The title compound was prepared using methods analogous to those described in Example 136. MS (ESI): mass calcd. for C₂₂H₂₅N₅O₂, 391.2. m/z found, 392.15 [M+H]⁺. ¹H NMR (400 MHz, CDCl₃) δ 9.21 (s, 1H), 8.29 (d, J=7.2 Hz, 1H), 8.10 (s, 1H), 7.67 (d, J=1.9 Hz, 1H), 7.17 (s, 1H), 7.04 (s, 1H), 6.97 (dd, J=7.2, 2.0 Hz, 1H), 4.51 (p, J=6.8 Hz, 1H), 2.66-2.58 (m, 2H), 2.55 (s, 3H), 2.12-2.00 (m, 2H), 1.65-1.56 (m, 1H), 1.31 (s, 3H), 1.20-1.11 (m, 2H), 0.89 (dq, J=7.4, 4.0 Hz, 2H).

### Example 518: 5-[5-[[(2S)-6,6-dimethylmorpholin-2-yl]methoxy]-2-methyl-4-pyridyl]-N-pyridazin-3-yl-pyrazolo[1,5-a]pyridin-2-amine

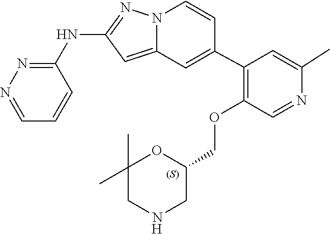

The title compound was prepared using methods analogous to those described in Example 116 Steps A and B. MS (ESI): mass calcd. for C₂₄H₂₇N₇O₂, 445.2. m/z found, 446.20 [M+H]⁺. ¹H NMR (500 MHz, CDCl₃) δ 10.55 (s, 1H), 8.66 (dd, J=4.5, 1.4 Hz, 1H), 8.30 (d, J=7.2 Hz, 1H), 8.21-8.09 (m, 3H), 7.60 (d, J=1.9 Hz, 1H), 7.31 (dd, J=9.1, 4.5 Hz, 1H), 7.08 (s, 1H), 6.89 (dd, J=7.2, 2.0 Hz, 1H), 6.72 (s, 1H), 4.00-3.90 (m, 2H), 3.88 (tt, J=7.2, 4.3 Hz, 1H), 2.85 (dd, J=12.1, 2.3 Hz, 1H), 2.56 (d, J=12.4 Hz, 1H), 2.55-2.46 (m, 1H), 1.90 (s, 2H), 1.22 (s, 3H), 1.10 (s, 5H).

### Example 519: (*S)—N-[5-[5-[(3-fluoro-1-methyl-pyrrolidin-3-yl)methoxy]-2-methyl-4-pyridyl]pyrazolo[1,5-a]pyridin-2-yl]cyclopropanecarboxamide

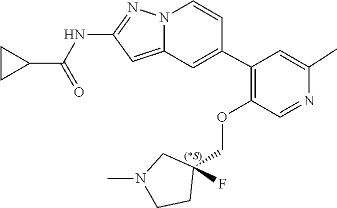

The title compound was prepared using methods analogous to those described in Example 136. MS (ESI): mass calcd. for C₂₃H₂₆FN₅O₂, 423.2. m/z found, 424.3 [M+H]⁺. ¹H NMR (400 MHz, CDCl₃) δ 8.42 (br s, 1H), 8.28 (d, J=7.2 Hz, 1H), 8.24 (s, 1H), 7.68 (d, J=1.2 Hz, 1H), 7.20 (s, 1H), 7.04-6.97 (m, 2H), 4.26-4.13 (m, 2H), 2.83-2.70 (m, 3H), 2.56 (s, 3H), 2.54-2.47 (m, 1H), 2.34 (s, 3H), 2.21-1.98 (m, 2H), 1.63-1.54 (m, 1H), 1.18-1.12 (m, 2H), 0.95-0.88 (m, 2H). ¹⁹F NMR (376 MHz, CDCl₃) δ −145.62 (s, 1F)

### Example 520: 5-[5-(3-aminocyclobutoxy)-2-methyl-4-pyridyl]-N-pyridazin-3-yl-pyrazolo[1,5-a]pyridin-2-amine

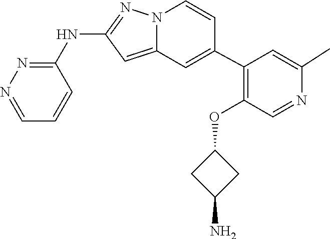 [SMILES: Cc1cc(-c2ccn3nc(Nc4cccnn4)cc3c2)c(O[C@H]2C[C@H](N)C2)cn1]

The title compound was prepared using methods analogous to those described in Example 116 Steps A and B. MS (ESI): mass calcd. for C₂₁H₂₁N₇O, 387.2. m/z found, 388.20 [M+H]⁺. ¹H NMR (500 MHz, CDCl₃) δ 10.53 (s, 1H), 8.67 (dd, J=4.4, 1.4 Hz, 1H), 8.32 (d, J=7.2 Hz, 1H), 8.11 (dt, J=9.2, 1.8 Hz, 1H), 7.95 (s, 1H), 7.55 (t, J=2.4 Hz, 1H), 7.30 (dd, J=9.1, 4.5 Hz, 1H), 7.08 (d, J=2.0 Hz, 1H), 6.83 (dt, J=7.3, 1.7 Hz, 1H), 6.73 (s, 1H), 2.83-2.75 (m, 2H), 2.45 (s, 3H), 2.36 (ddd, J=12.3, 7.5, 3.8 Hz, 2H), 2.15-2.06 (m, 2H), 1.83-1.72 (m, 2H).

### Example 521: N-(2,6-dimethylpyrimidin-4-yl)-5-[2-methyl-5-[[(2S)-1-methylazetidin-2-yl]methoxy]-4-pyridyl]pyrazolo[1,5-a]pyridin-2-amine

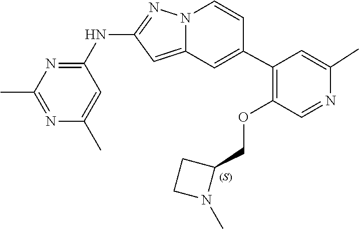

The title compound was prepared using a similar method described in Example 198. MS (ESI): mass calcd. for C₂₄H₂₇N₇O, 429.2. m/z found, 430.0 [M+H]⁺. ¹H NMR (500 MHz, DMSO-d₆) δ 10.18 (s, 1H), 8.59 (d, J=7.2 Hz, 1H), 8.33 (s, 1H), 7.89 (s, 1H), 7.36 (s, 1H), 7.05 (dd, J=7.3, 1.9 Hz, 1H), 7.01 (br s, 1H), 6.84 (br s, 1H), 4.14-4.06 (m, 2H), 3.30-3.24 (m, 2H), 2.77-2.68 (m, 1H), 2.46 (s, 3H), 2.45 (s, 3H), 2.30 (s, 3H), 2.17 (s, 3H), 2.00-1.85 (m, 2H).

### Example 522: (*R)—N-[5-[5-[(3-hydroxy-1-methyl-pyrrolidin-3-yl)methoxy]-2-methyl-4-pyridyl]pyrazolo[1,5-a]pyridin-2-yl]cyclopropanecarboxamide

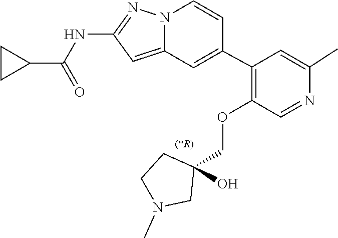

The title compound was prepared using methods analogous to those described in Example 136. MS (ESI): mass calcd. for C₂₃H₂₇N₅O₃, 421.2. m/z found, 422.3 [M+H]⁺. ¹H NMR (400 MHz, CDCl₃) δ 8.45 (s, 1H), 8.27 (s, 1H), 8.23 (d, J=7.2 Hz, 1H), 7.64 (d, J=0.8 Hz, 1H), 7.17 (s, 1H), 6.95 (s, 1H), 6.91 (dd, J=2.0, 7.2 Hz, 1H), 4.10 (s, 2H), 3.20 (br. s., 1H), 2.86-2.78 (m, 1H), 2.64 (s, 2H), 2.56 (s, 3H), 2.50-2.41 (m, 1H), 2.34 (s, 3H), 2.10-2.00 (m, 1H), 1.97-1.88 (m, 1H), 1.55-1.45 (m, 1H), 1.14-1.06 (m, 2H), 0.88-0.81 (m, 2H)

### Example 523: 5-[2-methyl-5-[[(1S,4S,7R)-2-oxa-5-azabicyclo[2.2.1]heptan-7-yl]oxy]-4-pyridyl]-N-(6-methylpyridazin-3-yl)pyrazolo[1,5-a]pyridin-2-amine

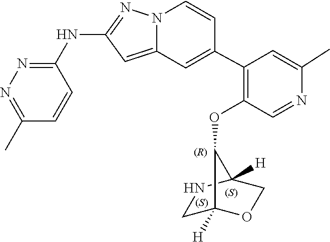

The title compound was prepared using methods analogous to those described in Example 116 Steps A and B. MS (ESI): mass calcd. for C₂₃H₂₃N₇O₂, 429.2. m/z found, 430.2 [M+H]⁺. ¹H NMR (400 MHz, CD₃OD) δ 8.44-8.39 (m, 2H), 7.75-7.72 (m, 1H), 7.68 (d, J=9.1 Hz, 1H), 7.43 (d, J=9.2 Hz, 1H), 7.41 (s, 1H), 7.00 (dd, J=7.2, 2.0 Hz, 1H), 6.88-6.86 (m, 1H), 4.89 (d, J=2.4 Hz, 1H), 4.37 (s, 1H), 4.05 (dd, J=7.9, 2.1 Hz, 1H), 3.90 (d, J=8.0 Hz, 1H), 3.61 (t, J=2.2 Hz, 1H), 3.17 (dd, J=11.2, 1.7 Hz, 1H), 3.00 (d, J=11.3, 0.8 Hz, 1H), 2.56 (d, J=5.1 Hz, 6H).

### Example 524: N-(2,6-dimethylpyrimidin-4-yl)-5-[2-methyl-5-[[(1S,4S,7R)-2-oxa-5-azabicyclo[2.2.1]heptan-7-yl]oxy]-4-pyridyl]pyrazolo[1,5-a]pyridin-2-amine

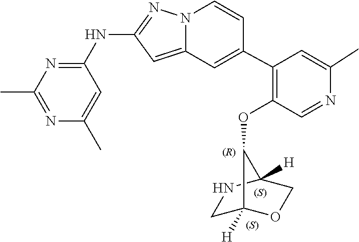

The title compound was prepared using a similar method described in Example 198. MS (ESI): mass calcd. for C₂₄H₂₅N₇O₂, 443.2. m/z found, 444.1 [M+H]⁺. ¹H NMR (500 MHz, CD₃OD) δ 8.41 (d, J=7.2 Hz, 1H), 8.39 (s, 1H), 7.74-7.69 (m, 1H), 7.37 (s, 1H), 7.01 (s, 1H), 6.99 (dd, J=7.2, 2.0 Hz, 1H), 6.84 (s, 1H), 4.88 (d, J=2.3 Hz, 1H), 4.37 (s, 1H), 4.04 (dd, J=7.9, 2.1 Hz, 1H), 3.90 (d, J=7.9 Hz, 1H), 3.60 (t, J=2.2 Hz, 1H), 3.16 (dd, J=11.2, 1.7 Hz, 1H), 3.00 (d, J=11.2 Hz, 1H), 2.54 (s, 3H), 2.52 (s, 3H), 2.37 (s, 3H).

### Example 525: N-(6-cyclopropylpyridazin-3-yl)-5-[2-methyl-5-[[(1S,4S,7R)-2-oxa-5-azabicyclo[2.2.1]heptan-7-yl]oxy]-4-pyridyl]pyrazolo[1,5-a]pyridin-2-amine

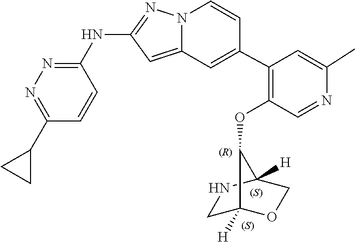

The title compound was prepared using methods analogous to those described in Example 116 Steps A and B. MS (ESI): mass calcd. for C₂₅H₂₅N₇O₂, 455.2. m/z found, 456.2 [M+H]⁺. ¹H NMR (500 MHz, CD₃OD) δ 8.42-8.38 (m, 2H), 7.74-7.71 (m, 1H), 7.64 (d, J=9.3 Hz, 1H), 7.40 (s, 1H), 7.28 (d, J=9.3 Hz, 1H), 6.99 (dd, J=7.2, 2.0 Hz, 1H), 6.86-6.82 (m, 1H), 4.87 (d, J=2.3 Hz, 1H), 4.35 (s, 1H), 4.05 (dd, J=7.8, 2.1 Hz, 1H), 3.88 (d, J=7.8 Hz, 1H), 3.55 (t, J=2.2 Hz, 1H), 3.14 (dd, J=11.1, 1.7 Hz, 1H), 2.97 (dd, J=11.1, 0.7 Hz, 1H), 2.55 (s, 3H), 2.21-2.12 (m, 1H), 1.11-1.04 (m, 2H), 1.00-0.94 (m, 2H).

### Example 526: N-[5-[5-[[(2S)-azetidin-2-yl]methoxy]-2-methyl-4-pyridyl]pyrazolo[1,5-a]pyridin-2-yl]cyclopropanecarboxamide

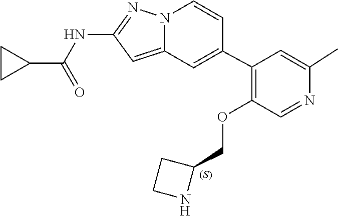

The title compound was prepared using a similar tosic acid displacement method described in Example 188, followed by a similar Boc-deprotection step described in Example 9, Step B. MS (ESI): mass calcd. for C₂₁H₂₃N₅O₂, 377.2. m/z found, 378.2 [M+H]⁺. ¹H NMR (400 MHz, CDCl₃) δ 8.48-8.52 (m, 1H), 8.28-8.23 (m, 2H), 7.71 (d, J=0.8 Hz, 1H), 7.17 (s, 1H), 6.99 (s, 1H), 6.97 (dd, J=2.0, 7.2 Hz, 1H), 4.27-4.19 (m, 1H), 4.12-4.08 (m, 2H), 3.71-3.62 (m, 1H), 3.44-3.33 (m, 1H), 2.55 (s, 3H), 2.38-2.17 (m, 2H), 1.63-1.51 (m, 1H), 1.22-1.07 (m, 2H), 0.99-0.83 (m, 2H)

### Example 527: N-(2,6-dimethylpyrimidin-4-yl)-5-[2-methyl-5-[[(2R)-1-methylazetidin-2-yl]methoxy]-4-pyridyl]pyrazolo[1,5-a]pyridin-2-amine

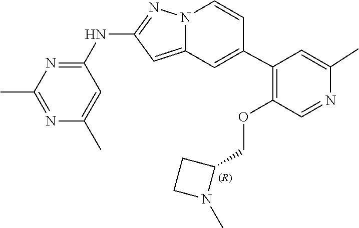

The title compound was prepared using a similar method described in Example 198. MS (ESI): mass calcd. for C₂₄H₂₇N₇O, 429.2. m/z found, 430.3 [M+H]⁺. ¹H NMR (500 MHz, CD₃OD) δ 8.37-8.32 (m, 1H), 8.14 (s, 1H), 7.71-7.66 (m, 1H), 7.27 (s, 1H), 6.98-6.88 (m, 2H), 6.76 (s, 1H), 4.11 (dd, J=9.8, 3.5 Hz, 1H), 4.01 (dd, J=9.8, 7.7 Hz, 1H), 3.49-3.39 (m, 1H), 3.34-3.25 (m, 1H), 2.88-2.77 (m, 1H), 2.43 (s, 3H), 2.43 (s, 3H), 2.28 (d, J=0.6 Hz, 3H), 2.16 (s, 3H), 2.05-1.89 (m, 2H).

### Example 528: 5-[2-methyl-5-[[(1S,4S,7R)-2-oxa-5-azabicyclo[2.2.1]heptan-7-yl]oxy]-4-pyridyl]-N-(5-methylpyrazin-2-yl)pyrazolo[1,5-a]pyridin-2-amine

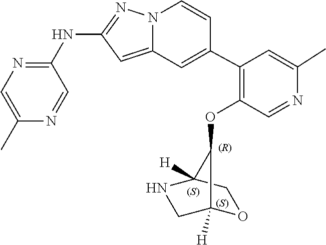

The title compound was prepared using methods analogous to those described in Example 116 Steps A and B. MS (ESI): mass calcd. for C₂₃H₂₃N₇O₂, 429.2. m/z found, 430.2 [M+H]⁺. ¹H NMR (600 MHz, CD₃OD) δ 8.53 (d, J=1.5 Hz, 1H), 8.42-8.39 (m, 1H), 8.39 (s, 1H), 8.13-8.11 (m, 1H), 7.73-7.69 (m, 1H), 7.39 (s, 1H), 6.97 (dd, J=7.2, 2.0 Hz, 1H), 6.79 (d, 1H), 4.88 (d, J=2.3 Hz, 1H), 4.37 (s, 1H), 4.05 (dd, J=8.0, 2.1 Hz, 1H), 3.90 (d, J=7.8 Hz, 1H), 3.60 (t, J=2.2 Hz, 1H), 3.16 (dd, J=11.2, 1.7 Hz, 1H), 3.00 (d, J=11.3, 0.8 Hz, 1H), 2.54 (s, 3H), 2.43 (s, 3H).

### Example 529: N-[5-[5-[[(2R)-1-(2-fluoroethyl)azetidin-2-yl]methoxy]-2-methyl-4-pyridyl]pyrazolo[1,5-a]pyridin-2-yl]cyclopropanecarboxamide

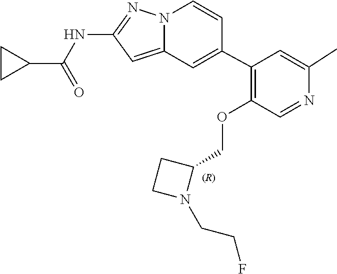

The title compound was prepared using methods analogous to those described in Example 457, Step B. MS (ESI): mass calcd. for C₂₃H₂₆FN₅O₂, 423.2. m/z found, 424.3 [M+H]t ¹H NMR (400 MHz, CDCl₃) δ 8.49 (s, 1H), 8.27 (d, J=7.2 Hz, 1H), 8.25 (s, 1H), 7.69 (s, 1H), 7.16 (s, 1H), 6.99 (s, 1H), 6.95 (dd, J=2.0, 7.2 Hz, 1H), 4.48-4.21 (m, 2H), 4.21-4.06 (m, 2H), 3.65-3.47 (m, 2H), 3.08-2.94 (m, 1H), 2.92-2.59 (m, 2H), 2.55 (s, 3H), 2.20-2.06 (m, 2H), 1.64-1.53 (m, 1H), 1.23-1.10 (m, 2H), 1.00-0.86 (m, 2H). ¹⁹F NMR (376 MHz, CDCl₃) δ −221.69 (s, 1F).

### Example 530: 6-[[5-[5-[[(1S,5R)-9-ethyl-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide

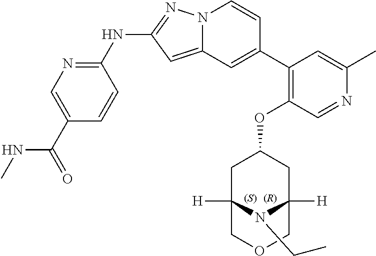

The title compound was prepared using methods analogous to those described in Example 116, using acetaldehyde in place of H(CHO)ₙ in step C. MS (ESI): mass calcd. for C₂₉H₃₃N₇O₃, 527.3. m/z found, 528.4 [M+H]⁺. ¹H NMR (400 MHz, CDCl₃) δ 8.67 (s, 1H), 8.33 (d, J=6.8 Hz, 1H), 8.17 (s, 1H), 8.07-7.99 (m, 2H), 7.56 (s, 1H), 7.50 (d, J=8.8 Hz, 1H), 7.23 (s, 1H), 7.20 (d, J=7.2 Hz, 1H), 6.54 (s, 1H), 6.12-6.03 (m, 1H), 4.67-4.58 (m, 1H), 3.97-3.89 (m, 2H), 3.70-3.62 (m, 2H), 3.06-2.99 (m, 3H), 2.82-2.78 (m, 2H), 2.69 (q, J=7.2 Hz, 2H), 2.55 (s, 3H), 2.43-2.32 (m, 2H), 1.84-1.76 (m, 2H), 1.08 (t, J=7.2 Hz, 3H)

### Example 531: 6-[[5-[5-[[(1S,5R)-9-isopropyl-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide

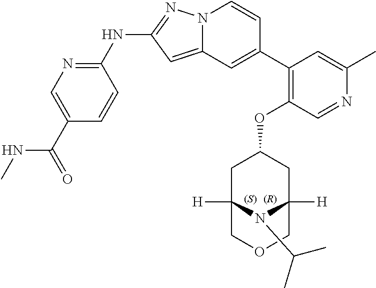

The title compound was prepared using methods analogous to those described in Example 116, using acetone in place of H(CHO)ₙ in step C. MS (ESI): mass calcd. for C₃₀H₃₅N₇O₃, 541.3. m/z found, 542.2 [M+H]⁺. ¹H NMR (400 MHz, CDCl₃) δ 8.66 (d, J=1.6 Hz, 1H), 8.32 (d, J=7.2 Hz, 1H), 8.15 (s, 1H), 8.09-7.98 (m, 2H), 7.54-7.43 (m, 2H), 7.23 (s, 1H), 7.20 (d, J=7.6 Hz, 1H), 6.54 (s, 1H), 6.12-5.99 (m, 1H), 4.70-4.55 (m, 1H), 3.97-3.84 (m, 2H), 3.80-3.69 (m, 2H), 3.12-2.89 (m, 6H), 2.55 (s, 3H), 2.41-2.18 (m, 2H), 1.91-1.72 (m, 2H), 1.17-0.97 (m, 6H)

### Example 532: 6-[[5-[5-[[(1S,5R)-9-(2-hydroxy-2-methyl-propyl)-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide

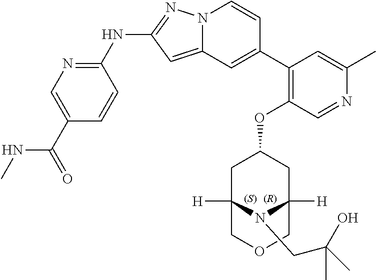

A mixture consisting of 6-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-N-methylnicotinamide (Example 173, 170 mg, 0.34 mmol), 2,2-dimethyloxirane (491 mg, 6.81 mmol), potassium carbonate (188 mg, 1.36 mmol), and DMF (3 mL) was stirred at 40° C. for 16 hr. The reaction mixture was purified by preparative HPLC using a Phenomenex Gemini-NX 150×30 mm×5 um column (eluent: 27% to 57% (v/v) CH₃CN and H₂O with 0.04% NH₃) to afford pure product. The product was suspended in water (10 mL), the mixture frozen using dry ice/ethanol, and then lyophilized to dryness to afford the title compound as a light yellow solid (70 mg, 35%). MS (ESI): mass calcd. for C₃₁H₃₇N₇O₄, 571.3. m/z found, 572.2 [M+H]⁺. ¹H NMR (400 MHz, CDCl₃) δ 8.68 (d, J=2.0 Hz, 1H), 8.33 (d, J=7.2 Hz, 1H), 8.19 (s, 1H), 8.04 (dd, J=2.4, 8.8 Hz, 1H), 7.97 (d, J=1.2 Hz, 1H), 7.65 (s, 1H), 7.48 (d, J=8.8 Hz, 1H), 7.22 (s, 1H), 7.14 (dd, J=2.0, 7.2 Hz, 1H), 6.56 (s, 1H), 6.11 (q, J=4.8 Hz, 1H), 4.69-4.53 (m, 1H), 3.99-3.86 (m, 2H), 3.72-3.63 (m, 2H), 3.03 (d, J=4.8 Hz, 3H), 2.92 (s, 1H), 2.79-2.72 (m, 2H), 2.56 (s, 3H), 2.52 (s, 2H), 2.45-2.33 (m, 2H), 1.96-1.81 (m, 2H), 1.14 (s, 6H)

### Example 533: (*S)—N-(2,6-dimethylpyrimidin-4-yl)-5-[5-(5,5-dimethyltetrahydrofuran-3-yl)oxy-2-methyl-4-pyridyl]pyrazolo[1,5-a]pyridin-2-amine

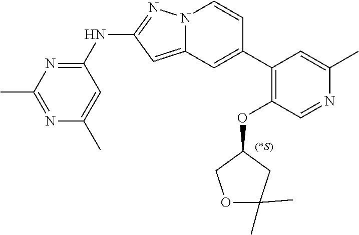

The title compound was prepared using a similar method described in Example 198. Purified by SFC over DAICEL CHIRALPAK AD 250 mm×30 mm×10 μm (eluent: 45% to 45% (v/v) EtOH with 0.1% NH₃·H₂O in supercritical CO₂). MS (ESI): mass calcd. for C₂₅H₂₈N₆O₂, 444.2. m/z found, 445.1 [M+H]⁺. ¹H NMR (400 MHz, DMSO-d₆) δ 10.20 (s, 1H), 8.58 (d, J=7.2 Hz, 1H), 8.27 (s, 1H), 7.81 (d, J=0.8 Hz, 1H), 7.36 (s, 1H), 7.05-6.98 (m, 2H), 6.86 (s, 1H), 5.22-5.16 (m, 1H), 4.05 (dd, J=4.8, 10.4 Hz, 1H), 3.85 (d, J=10.8 Hz, 1H), 2.47 (s, 3H), 2.46 (s, 3H), 2.30 (s, 3H), 2.15 (dd, J=6.8, 13.6 Hz, 1H), 1.89 (d, J=14.0 Hz, 1H), 1.19 (s, 3H), 1.18 (s, 3H)

### Example 534: (3*R, 4*R)—N-(2,6-dimethylpyrimidin-4-yl)-5-[2-methyl-5-(4-methyltetrahydrofuran-3-yl)oxy-4-pyridyl]pyrazolo[1,5-a]pyridin-2-amine

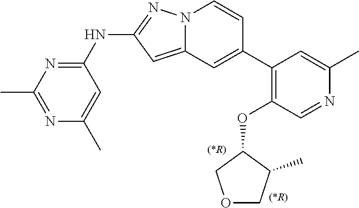

The title compound was prepared using a similar method described in Example 203. Purified by SFC over AD 250 mm×30 mm, 10 μm (eluent: 35% to 35% (v/v) supercritical CO₂ in EtOH and H₂O with 0.1% NH₃). MS (ESI): mass calcd. for C₂₄H₂₆N₆O₂, 430.2. m/z found, 431.1 [M+H]⁺. ¹H NMR (400 MHz, CDCl₃) δ 8.37 (d, J=7.2 Hz, 1H), 8.15 (s, 1H), 7.60 (d, J=0.8 Hz, 1H), 7.52 (s, 1H), 7.19 (s, 1H), 7.09 (s, 1H), 6.93 (dd, J=2.0, 7.6 Hz, 1H), 6.57 (s, 1H), 4.87-4.82 (m, 1H), 4.09 (dd, J=4.0, 10.4 Hz, 1H), 4.04-3.99 (m, 1H), 3.96 (dd, J=1.2, 10.4 Hz, 1H), 3.51 (dd, J=8.4, 10.4 Hz, 1H), 2.61 (s, 3H), 2.56 (s, 3H), 2.53-2.47 (m, 1H), 2.45 (s, 3H), 1.04 (d, J=6.8 Hz, 3H).

### Example 535: (*R)—N-(2,6-dimethylpyrimidin-4-yl)-5-[2-methyl-5-[(3-methyltetrahydrofuran-3-yl)methoxy]-4-pyridyl]pyrazolo[1,5-a]pyridin-2-amine

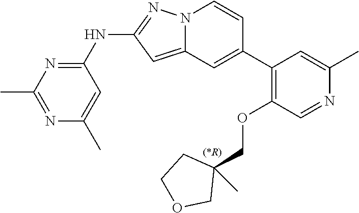

The title compound was prepared using a similar method described in Example 203. Purified by SFC over DAICEL CHIRALCEL OJ-H 250 mm×30 mm, 5 μm (eluent: 35% to 35% (v/v) supercritical 0.1% NH₃—H₂O EtOH). MS (ESI): mass calcd. for C₂₅H₂₈N₆O₂, 444.2. m/z found, 445.1 [M+H]⁺. ¹H NMR (400 MHz, CDCl₃) δ 8.41 (d, J=7.2 Hz, 1H), 8.25 (s, 1H), 7.82 (br. s., 1H), 7.61 (s, 1H), 7.18 (s, 1H), 7.10 (s, 1H), 6.93 (dd, J=1.6, 7.2 Hz, 1H), 6.57 (s, 1H), 3.92 (s, 2H), 3.89-3.82 (m, 2H), 3.74 (d, J=8.8 Hz, 1H), 3.40 (d, J=8.4 Hz, 1H), 2.61 (s, 3H), 2.56 (s, 3H), 2.45 (s, 3H), 1.92-1.85 (m, 1H), 1.71-1.62 (m, 1H), 1.18 (s, 3H).

### Example 536: (*S)—N-(2,6-dimethylpyrimidin-4-yl)-5-[2-methyl-5-[(3-methyltetrahydrofuran-3-yl)methoxy]-4-pyridyl]pyrazolo[1,5-a]pyridin-2-amine

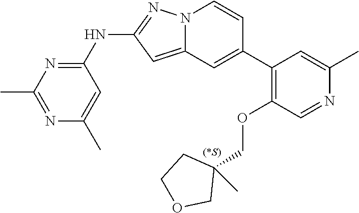

The title compound was prepared using a similar method described in Example 203. Purified by SFC over DAICEL CHIRALCEL OJ-H 250 mm×30 mm, 5 μm (eluent: 35% to 35% (v/v) supercritical 0.1% NH₃—H₂O EtOH). MS (ESI): mass calcd. for C₂₅H₂₈N₆O₂, 444.2. m/z found, 445.2 [M+H]⁺. ¹H NMR (400 MHz, DMSO-d₆) δ 10.21 (s, 1H), 8.61 (d, J=7.6 Hz, 1H), 8.34 (s, 1H), 7.79 (d, J=0.8 Hz, 1H), 7.36 (s, 1H), 7.04 (br. s., 1H), 7.00 (dd, J=1.6, 6.8 Hz, 1H), 6.84 (br. s., 1H), 3.98 (s, 2H), 3.77-3.70 (m, 2H), 3.62 (d, J=8.4 Hz, 1H), 3.31 (d, J=8.4 Hz, 1H), 2.47 (s, 3H), 2.46 (s, 3H), 2.31 (s, 3H), 1.91-1.82 (m, 1H), 1.63-1.56 (m, 1H), 1.10 (s, 3H)

### Example 537: 2-methyl-1-[[6-methyl-4-[2-[[6-methyl-2-(trifluoromethyl)pyrimidin-4-yl]amino]pyrazolo[1,5-a]pyridin-5-yl]-3-pyridyl]oxy]propan-2-ol

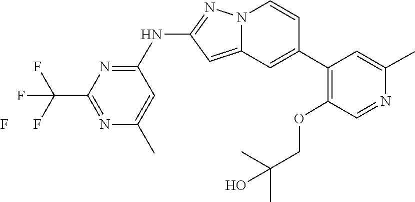

The title compound was prepared using a similar method described in Example 200. MS (ESI): mass calcd. for C₂₃H₂₃F₃N₆O₂, 472.2. m/z found, 473.2 [M+H]⁺. ¹H NMR (500 MHz, DMSO-d6) δ 10.92 (s, 1H), 8.68-8.61 (m, 1H), 8.33 (s, 1H), 7.99-7.89 (m, 1H), 7.39 (s, 2H), 7.15 (dd, J=7.2, 2.0 Hz, 1H), 6.84 (s, 1H), 4.63 (s, 1H), 3.88 (s, 2H), 2.47 (s, 3H), 2.46 (s, 3H), 1.16 (s, 6H).

### Example 538: (1r,3r)-3-(((4-(2-((2,6-dimethylpyrimidin-4-yl)amino)pyrazolo[1,5-a]pyridin-5-yl)-6-methylpyridin-3-yl)oxy)methyl)cyclobutan-1-ol

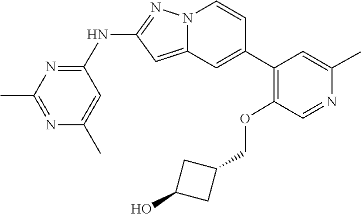

The title compound was prepared using a similar method described in Example 203. MS (ESI): mass calcd. for C₂₄H₂₆N₆O₂, 430.2. m/z found, 431.1 [M+H]⁺. ¹H NMR (400 MHz, CDCl₃) δ 8.39 (d, J=7.2 Hz, 1H), 8.25 (s, 1H), 7.73 (s, 1H), 7.65-7.57 (m, 1H), 7.18 (s, 1H), 7.08 (s, 1H), 6.94 (dd, J=1.6, 7.2 Hz, 1H), 6.55 (s, 1H), 4.44-4.32 (m, 1H), 4.06 (d, J=6.4 Hz, 2H), 2.72-2.62 (m, 1H), 2.59 (s, 3H), 2.56 (s, 3H), 2.44 (s, 3H), 2.30-2.18 (m, 2H), 2.17-2.06 (m, 2H).

### Example 539: (*R)—N-(2,6-dimethylpyrimidin-4-yl)-5-[5-[(5,5-dimethyltetrahydrofuran-3-yl)methoxy]-2-methyl-4-pyridyl]pyrazolo[1,5-a]pyridin-2-amine

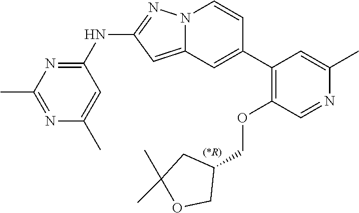

The title compound was prepared using a similar method described in Example 203. Purified by SFC with a DAICEL CHIRALPAK IG (250 mm×30 mm, 10 um) column (eluent: 55% to 55% (v/v) EtOH with 0.1% NH₃—H₂O in supercritical CO₂). MS (ESI): mass calcd. for C₂₆H₃₀N₆O₂, 458.2. m/z found, 459.2 [M+H]⁺. ¹H NMR (400 MHz, CDCl₃) δ 8.39 (d, J=6.8 Hz, 1H), 8.25 (s, 1H), 7.63 (s, 1H), 7.61 (d, J=1.2 Hz, 1H), 7.18 (s, 1H), 7.08 (s, 1H), 6.92 (dd, J=1.6, 7.2 Hz, 1H), 6.58 (s, 1H), 4.11-4.04 (m, 1H), 4.03-3.94 (m, 2H), 3.73-3.65 (m, 1H), 2.88-2.74 (m, 1H), 2.61 (s, 3H), 2.56 (s, 3H), 2.45 (s, 3H), 1.96-1.89 (m, 1H), 1.54-1.47 (m, 1H), 1.28 (s, 3H), 1.21 (s, 3H).

### Example 540: (*S)—N-(2,6-dimethylpyrimidin-4-yl)-5-[5-[(5,5-dimethyltetrahydrofuran-3-yl)methoxy]-2-methyl-4-pyridyl]pyrazolo[1,5-a]pyridin-2-amine

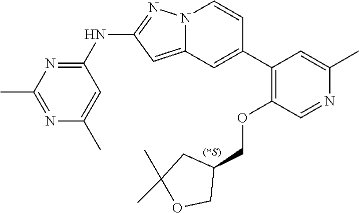

The title compound was prepared using a similar method described in Example 203. Purified by SFC with a DAICEL CHIRALPAK IG (250 mm×30 mm, 10 um) column (eluent: 55% to 55% (v/v) EtOH with 0.1% NH₃—H₂O in supercritical CO₂). MS (ESI): mass calcd. for C₂₆H₃₀N₆O₂, 458.2. m/z found, 459.2 [M+H]⁺. ¹H NMR (400 MHz, CDCl₃) δ 8.38 (d, J=7.6 Hz, 1H), 8.25 (s, 1H), 7.61 (d, J=0.8 Hz, 1H), 7.59 (s, 1H), 7.18 (s, 1H), 7.08 (s, 1H), 6.92 (dd, J=1.6, 6.8 Hz, 1H), 6.58 (s, 1H), 4.11-4.05 (m, 1H), 4.03-3.94 (m, 2H), 3.72-3.65 (m, 1H), 2.87-2.75 (m, 1H), 2.61 (s, 3H), 2.56 (s, 3H), 2.45 (s, 3H), 1.96-1.89 (m, 1H), 1.55-1.47 (m, 1H), 1.28 (s, 3H), 1.21 (s, 3H).

### Example 541: 2-methyl-1-[[6-methyl-4-[2-[(5-methylpyrazin-2-yl)amino]pyrazolo[1,5-a]pyridin-5-yl]-3-pyridyl]oxy]propan-2-ol

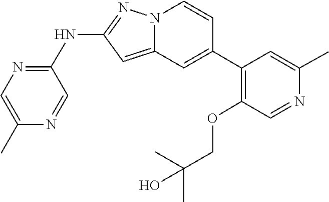 [SMILES: Cc1cnc(Nc2cc3cc(-c4cc(C)ncc4OCC(C)(C)O)ccn3n2)cn1]

The title compound was prepared using a similar method described in Example 200. MS (ESI): mass calcd. for C₂₂H₂₄N₆O₂, 404.2. m/z found, 405.20 [M+H]⁺. ¹H NMR (500 MHz, Chloroform-d) δ 8.61 (d, J=8.3 Hz, 1H), 8.40 (d, J=1.6 Hz, 1H), 8.24-8.19 (m, 1H), 8.14 (s, 1H), 7.94 (s, 1H), 7.46-7.42 (m, 1H), 7.04 (s, 1H), 6.78 (dd, J=7.1, 1.9 Hz, 1H), 6.56 (s, 1H), 3.84 (s, 2H), 3.39 (d, J=0.7 Hz, 1H), 2.44 (s, 3H), 2.33 (s, 3H), 1.23 (s, 6H).

### Example 542: (rac-)-4-[[4-[2-[(2,6-dimethylpyrimidin-4-yl)amino]pyrazolo[1,5-a]pyridin-5-yl]-6-methyl-3-pyridyl]oxymethyl]-1-methyl-pyrrolidin-2-one

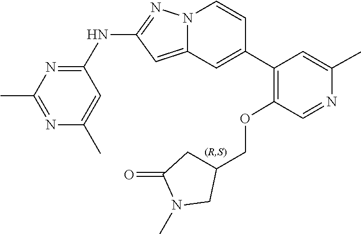

The title compound was prepared using a similar method described in Example 203. MS (ESI): mass calcd. for C₂₅H₂₇N₇O₂, 457.2. m/z found, 458.2 [M+H]⁺. ¹H NMR (500 MHz, DMSO-d6) δ 10.20 (s, 1H), 8.57 (d, J=7.1 Hz, 1H), 8.34 (s, 1H), 7.83-7.72 (m, 1H), 7.37 (s, 1H), 7.06-6.95 (m, 2H), 6.88 (s, 1H), 4.12 (d, J=6.3 Hz, 2H), 3.45 (dd, J=9.8, 8.5 Hz, 1H), 3.19 (dd, J=9.8, 5.6 Hz, 1H), 2.82-2.71 (m, 1H), 2.66 (s, 3H), 2.46 (d, J=3.1 Hz, 6H), 2.43-2.34 (m, 1H), 2.30 (s, 3H), 2.14 (dd, J=16.7, 6.6 Hz, 1H).

### Example 543: (*R)—N-(2,6-dimethylpyrimidin-4-yl)-5-[5-(6,9-dioxaspiro[4.4]nonan-8-ylmethoxy)-2-methyl-4-pyridyl]pyrazolo[1,5-a]pyridin-2-amine

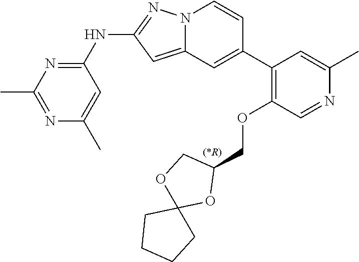

The title compound was prepared using a similar method described in Example 203. Purified by SFC with a DAICEL CHIRALCEL OJ-H (250 mm×30 mm, 5 um) column (eluent: 45% to 45% (v/v) EtOH with 0.1% NH₃—H₂O in supercritical CO₂). MS (ESI): mass calcd. for C₂₇H₃₀N₆O₃, 486.2. m/z found, 487.2 [M+H]⁺. ¹H NMR (400 MHz, CDCl₃) δ 8.39 (d, J=7.2 Hz, 1H), 8.29 (s, 1H), 7.70-7.62 (m, 2H), 7.19 (s, 1H), 7.07 (s, 1H), 6.95 (dd, J=2.0, 7.6 Hz, 1H), 6.59 (s, 1H), 4.43-4.29 (m, 1H), 4.20-4.06 (m, 2H), 4.04-3.94 (m, 1H), 3.85-3.76 (m, 1H), 2.61 (s, 3H), 2.57 (s, 3H), 2.45 (s, 3H), 1.81-1.72 (m, 4H), 1.70-1.61 (m, 4H).

### Example 544: (*S)—N-(2,6-dimethylpyrimidin-4-yl)-5-[5-(6,9-dioxaspiro[4.4]nonan-8-ylmethoxy)-2-methyl-4-pyridyl]pyrazolo[1,5-a]pyridin-2-amine

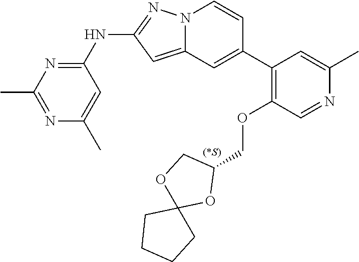

The title compound was prepared using a similar method described in Example 203. Purified by SFC with a DAICEL CHIRALCEL OJ-H (250 mm×30 mm, 5 um) column (eluent: 45% to 45% (v/v) EtOH with 0.1% NH₃—H₂O in supercritical CO₂). MS (ESI): mass calcd. for C₂₇H₃₀N₆O₃, 486.2. m/z found, 487.1 [M+H]⁺. ¹H NMR (400 MHz, CDCl₃) δ 8.37 (d, J=7.2 Hz, 1H), 8.29 (s, 1H), 7.67 (d, J=0.8 Hz, 1H), 7.56 (s, 1H), 7.19 (s, 1H), 7.07 (s, 1H), 6.95 (dd, J=1.6, 7.2 Hz, 1H), 6.58 (s, 1H), 4.40-4.32 (m, 1H), 4.18-4.06 (m, 2H), 4.03-3.96 (m, 1H), 3.84-3.77 (m, 1H), 2.61 (s, 3H), 2.57 (s, 3H), 2.45 (s, 3H), 1.84-1.78 (m, 4H), 1.72-1.63 (m, 4H).

### Example 545: 5-(5-((1r,3r)-3-aminocyclobutoxy)-2-methylpyridin-4-yl)-N-(2,6-dimethylpyrimidin-4-yl)pyrazolo[1,5-a]pyridin-2-amine

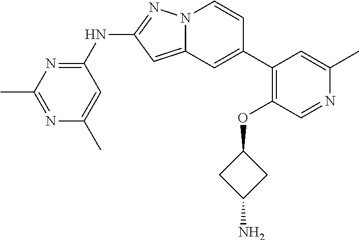

The title compound was prepared using a similar method described in Example 203. MS (ESI): mass calcd. for C₂₃H₂₅N₇O, 415.2. m/z found, 416.2 [M+H]⁺. ¹H NMR (400 MHz, DMSO-d₆) δ 10.22 (s, 1H), 8.59 (d, J=7.2 Hz, 1H), 8.06 (s, 1H), 7.81 (s, 1H), 7.36 (s, 1H), 7.12-6.95 (m, 2H), 6.88 (br. s., 1H), 5.04-4.91 (m, 1H), 3.61-3.51 (m, 1H), 2.47 (s, 3H), 2.46 (s, 3H), 2.31 (s, 3H), 2.28-2.23 (m, 2H), 2.20-2.10 (m, 2H).

### Example 546: N-(2,6-dimethylpyrimidin-4-yl)-5-[2-methyl-5-[(3-methyloxetan-3-yl)methoxy]-4-pyridyl]pyrazolo[1,5-a]pyridin-2-amine

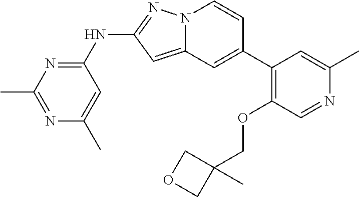

The title compound was prepared using a similar method described in Example 198. MS (ESI): mass calcd. for C₂₄H₂₆N₆O₂, 430.2. m/z found, 431.2 [M+H]⁺. ¹H NMR (400 MHz, DMSO-d6) δ 10.19 (s, 1H), 8.58 (dt, J=7.2, 0.9 Hz, 1H), 8.39 (s, 1H), 7.81 (dd, J=2.0, 0.9 Hz, 1H), 7.38 (s, 1H), 7.03 (s, 1H), 7.00 (dd, J=7.2, 2.0 Hz, 1H), 6.82 (s, 1H), 4.47 (d, J=5.8 Hz, 2H), 4.25 (d, J=5.8 Hz, 2H), 4.20 (s, 2H), 2.48 (s, 3H), 2.45 (s, 3H), 2.30 (s, 3H), 1.30 (s, 3H).

### Example 547: (*R)-4-[[4-[2-[(2,6-dimethylpyrimidin-4-yl)amino]pyrazolo[1,5-a]pyridin-5-yl]-6-methyl-3-pyridyl]oxymethyl]-1-methyl-pyrrolidin-2-one

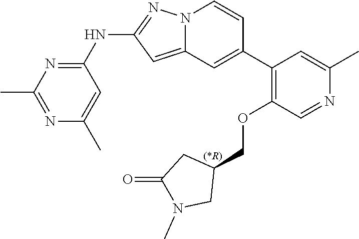

The title compound was obtained by chiral purification (chiral SFC AS-H (2×25 cm) 25% ethanol (0.1% DEA)/CO₂, 100 bar) of Example 542. MS (ESI): mass calcd. for C₂₅H₂₇N₇O₂, 457.2. m/z found, 458.2 [M+H]⁺. ¹H NMR (500 MHz, DMSO-d₆) δ 10.19 (s, 1H), 8.57 (dt, J=7.3, 0.9 Hz, 1H), 8.34 (s, 1H), 7.78 (dd, J=2.0, 0.9 Hz, 1H), 7.37 (s, 1H), 7.01 (s, 1H), 6.98 (dd, J=7.2, 2.0 Hz, 1H), 6.87 (s, 1H), 4.12 (d, J=6.4 Hz, 2H), 3.52-3.39 (m, 1H), 3.19 (dd, J=9.9, 5.6 Hz, 1H), 2.82-2.70 (m, 1H), 2.65 (s, 3H), 2.46 (d, J=4.0 Hz, 6H), 2.39 (dd, J=16.7, 9.5 Hz, 1H), 2.30 (s, 3H), 2.13 (dd, J=16.8, 6.6 Hz, 1H).

### Example 548: (*R)-2-[[4-[2-[(2,6-dimethylpyrimidin-4-yl)amino]pyrazolo[1,5-a]pyridin-5-yl]-6-methyl-3-pyridyl]oxy]-1-(1-methylcyclopropyl)ethanol

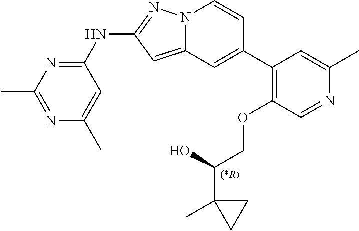

Step A: 2-((4-(2-((2,6-dimethylpyrimidin-4-yl)amino)pyrazolo[1,5-a]pyridin-5-yl)-6-methylpyridin-3-yl)oxy)-1-(1-methylcyclopropyl)ethan-1-one. The title compound was prepared using methods analogous to those described in Example 188 and using Intermediate 16 instead of Intermediate 7 and using 2-chloro-1-(1-methylcyclopropyl)ethan-1-one instead of 4,4-difluoro-(1-hydroxycyclohexyl)methyl 4-methylbenzenesulfonate.

Step B: 2-((4-(2-((2,6-dimethylpyrimidin-4-yl)amino)pyrazolo[1,5-a]pyridin-5-yl)-6-methylpyridin-3-yl)oxy)-1-(1-methylcyclopropyl)ethan-1-ol. To a solution of NaBH₄ (87.2 mg, 2.31 mmol) in MeOH (3 mL) was added 2-((4-(2-((2,6-dimethylpyrimidin-4-yl)amino)pyrazolo[1,5-a]pyridin-5-yl)-6-methylpyridin-3-yl)oxy)-1-(1-methylcyclopropyl)ethan-1-one (170 mg, 0.384 mmol) slowly at 0° C. The mixture was stirred for 40 minutes at r.t. The reaction mixture was directly concentrated to afford the crude product, which was purified by preparative HPLC using a Phenomenex Gemini NX-C18 75×30 mm×3 μm column (eluent: 28% to 58% (v/v) CH₃CN and NH₃H₂O with 10 mM NH₄HCO₃) to afford pure product. The product was suspended in water (10 mL), the mixture frozen using dry ice/ethanol, and then lyophilized to dryness to afford the title compound (100 mg, 59%) as a white solid.

Step C: (*R)-2-[[4-[2-[(2,6-dimethylpyrimidin-4-yl)amino]pyrazolo[1,5-a]pyridin-5-yl]-6-methyl-3-pyridyl]oxy]-1-(1-methylcyclopropyl)ethanol. An SFC purification step, similar to that described in Example 5, was performed on 2-((4-(2-((2,6-dimethylpyrimidin-4-yl)amino)pyrazolo[1,5-a]pyridin-5-yl)-6-methylpyridin-3-yl)oxy)-1-(1-methylcyclopropyl)ethan-1-ol to yield the title compound. MS (ESI): mass calcd. for C₂₅H₂₈N₆O₂, 444.2. m/z found, 445.2 [M+H]⁺. ¹H NMR (400 MHz, DMSO-d6) δ 10.21 (s, 1H), 8.57 (d, J=7.2 Hz, 1H), 8.36 (s, 1H), 7.89 (d, J=0.8 Hz, 1H), 7.36 (s, 1H), 7.09 (dd, J=2.0, 7.2 Hz, 1H), 7.02 (s, 1H), 6.85 (s, 1H), 4.95 (d, J=4.8 Hz, 1H), 4.22-4.14 (m, 1H), 4.12-4.03 (m, 1H), 3.23-3.15 (m, 1H), 2.47 (s, 3H), 2.46 (s, 3H), 2.30 (s, 3H), 1.00 (s, 3H), 0.45-0.37 (m, 2H), 0.23-0.14 (m, 2H).

### Example 549: (*S)-2-[[4-[2-[(2,6-dimethylpyrimidin-4-yl)amino]pyrazolo[1,5-a]pyridin-5-yl]-6-methyl-3-pyridyl]oxy]-1-(1-methylcyclopropyl)ethanol

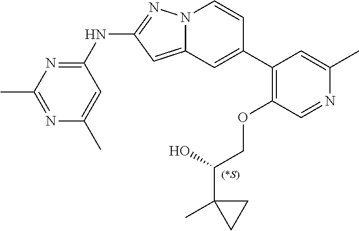

The title compound is the other isolated isomer from the SFC purification step in Example 548, Step C. MS (ESI): mass calcd. for C₂₅H₂₈N₆O₂, 444.2. m/z found, 445.1 [M+H]⁺. ¹H NMR (400 MHz, DMSO-d6) δ 10.21 (s, 1H), 8.57 (d, J=7.2 Hz, 1H), 8.36 (s, 1H), 7.89 (d, J=1.2 Hz, 1H), 7.36 (s, 1H), 7.08 (dd, J=2.0, 7.2 Hz, 1H), 7.02 (s, 1H), 6.84 (s, 1H), 4.95 (d, J=4.4 Hz, 1H), 4.22-4.13 (m, 1H), 4.11-4.03 (m, 1H), 3.22-3.14 (m, 1H), 2.46 (s, 3H), 2.46 (s, 3H), 2.30 (s, 3H), 1.00 (s, 3H), 0.45-0.37 (m, 2H), 0.22-0.14 (m, 2H).

### Example 550: N-(2,6-dimethylpyrimidin-4-yl)-5-[5-(1,1-dioxothian-4-yl)oxy-2-methyl-4-pyridyl]pyrazolo[1,5-a]pyridin-2-amine

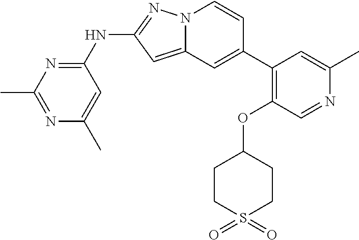

The title compound was prepared using a similar method described in Example 198. MS (ESI): mass calcd. for C₂₄H₂₆N₆O₃S, 478.2. m/z found, 479.15 [M+H]⁺. ¹H NMR (500 MHz, CDCl₃) δ 8.48 (dt, J=7.2, 0.9 Hz, 1H), 8.41 (s, 1H), 8.29 (s, 1H), 7.58 (dd, J=1.9, 0.9 Hz, 1H), 7.21 (s, 1H), 7.06 (s, 1H), 6.89 (dd, J=7.2, 1.9 Hz, 1H), 6.67 (s, 1H), 4.56 (tt, J=4.7, 2.4 Hz, 1H), 3.19-3.09 (m, 2H), 2.92-2.83 (m, 2H), 2.59 (d, J=16.0 Hz, 6H), 2.44 (s, 3H), 2.44-2.26 (m, 4H).

### Example 551: N-(2,6-dimethylpyrimidin-4-yl)-5-[2-methyl-5-(4-methylsulfonylcyclohexoxy)-4-pyridyl]pyrazolo[1,5-a]pyridin-2-amine

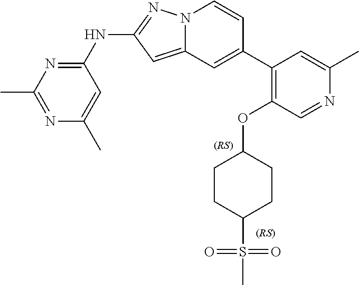

The title compound was prepared using a similar method described in Example 198. MS (ESI): mass calcd. for C₂₆H₃₀N₆O3S, 506.2. m/z found, 507.20 [M+H]⁺. ¹H NMR (500 MHz, CDCl₃) δ 8.57 (s, 1H), 8.47 (dd, J=13.6, 7.1 Hz, 1H), 8.25 (d, J=10.7 Hz, 1H), 7.64-7.59 (m, 1H), 7.18 (d, J=6.1 Hz, 1H), 7.01 (s, 1H), 6.96-6.89 (m, 1H), 6.64 (s, 1H), 3.46 (s, 3H), 2.81 (d, J=2.6 Hz, 3H), 2.61-2.51 (m, 7H), 2.42 (d, J=2.8 Hz, 3H), 2.31-2.17 (m, 2H), 1.98 (dd, J=13.2, 3.7 Hz, 1H), 1.84-1.74 (m, 1H), 1.69-1.51 (m, 2H).

### Example 552: 4-[[4-[2-[(2,6-dimethylpyrimidin-4-yl)amino]pyrazolo[1,5-a]pyridin-5-yl]-6-methyl-3-pyridyl]oxy]cyclohexanecarbonitrile

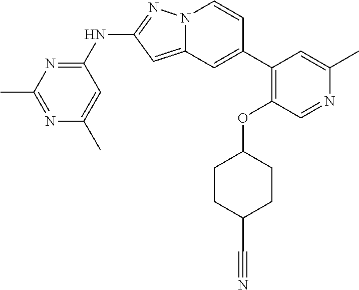

The title compound was prepared using a similar method described in Example 198. MS (ESI): mass calcd. for C₂₆H₂₇N₇O, 453.2. m/z found, 454.20 [M+H]⁺. ¹H NMR (500 MHz, CDCl₃) δ 8.55-8.46 (m, 2H), 8.26 (d, J=10.4 Hz, 1H), 7.64-7.60 (m, 1H), 7.19 (d, J=6.1 Hz, 1H), 7.05 (d, J=18.4 Hz, 1H), 6.95 (td, J=7.1, 1.9 Hz, 1H), 6.63 (s, 1H), 4.44-4.30 (m, 1H), 2.61 (d, J=2.1 Hz, 4H), 2.56 (s, 3H), 2.44 (s, 3H), 2.06-1.98 (m, 1H), 2.02-1.93 (m, 1H), 1.96-1.89 (m, 3H), 1.83-1.60 (m, 3H).

### Example 553: 5-[5-[[2-(difluoromethoxy)phenyl]methoxy]-2-methyl-4-pyridyl]-N-(2,6-dimethylpyrimidin-4-yl)pyrazolo[1,5-a]pyridin-2-amine

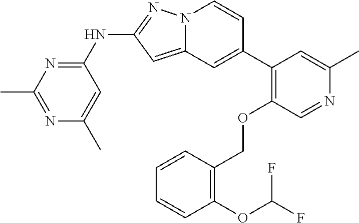

The title compound was prepared using a similar method described in Example 198. MS (ESI): mass calcd. for C₂₇H₂₄F₂N₆O₂, 502.2. m/z found, 503.2 [M+H]⁺. ¹H NMR (500 MHz, CDCl₃) δ 8.36 (d, J=7.4 Hz, 2H), 7.95 (s, 1H), 7.65 (d, J=1.9 Hz, 1H), 7.40 (dd, J=7.6, 1.7 Hz, 1H), 7.36-7.28 (m, 1H), 7.23-7.09 (m, 4H), 6.97 (dd, J=7.1, 2.0 Hz, 1H), 6.67-6.31 (m, 2H), 5.21 (s, 2H), 2.63 (s, 3H), 2.57 (s, 3H), 2.48 (s, 3H).

### Example 554: (3S)-4-[[4-[2-[(2,6-dimethylpyrimidin-4-yl)amino]pyrazolo[1,5-a]pyridin-5-yl]-6-methyl-3-pyridyl]oxy]-2,3-dimethyl-butan-2-ol

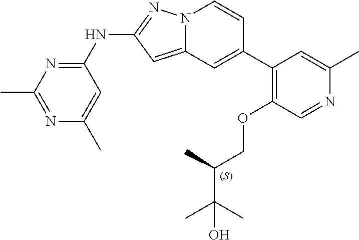

The title compound was prepared using a similar method described in Example 198. MS (ESI): mass calcd. for C₂₅H₃₀N₆O₂, 446.2. m/z found, 447.25 [M+H]⁺. ¹H NMR (400 MHz, CDCl₃) δ 8.55-8.44 (m, 2H), 8.30 (s, 1H), 7.58 (dd, J=1.9, 0.9 Hz, 1H), 7.15 (s, 1H), 7.04 (s, 1H), 6.91 (dd, J=7.2, 1.9 Hz, 1H), 6.58 (s, 1H), 5.29 (s, 1H), 4.26 (dd, J=9.1, 5.1 Hz, 1H), 4.03 (dd, J=9.1, 6.5 Hz, 1H), 2.60 (s, 3H), 2.55 (s, 3H), 2.43 (s, 3H), 1.99 (pd, J=7.0, 5.1 Hz, 1H), 1.21 (s, 3H), 1.15 (s, 3H), 1.03 (d, J=7.0 Hz, 3H).

### Example 555: 4-[[4-[2-[(2,6-dimethylpyrimidin-4-yl)amino]pyrazolo[1,5-a]pyridin-5-yl]-6-methyl-3-pyridyl]oxy]-2-methyl-butan-2-ol

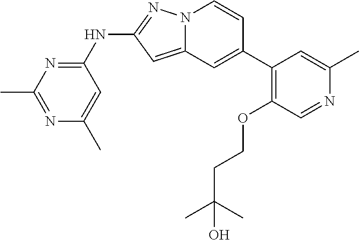

The title compound was prepared using a similar method described in Example 198. MS (ESI): mass calcd. for C₂₄H₂₈N₆O₂, 432.2. m/z found, 433.20 [M+H]⁺. ¹H NMR (400 MHz, CDCl₃) δ 8.41-8.33 (m, 1H), 8.24 (d, J=3.5 Hz, 1H), 7.63 (q, J=2.5 Hz, 1H), 7.35 (q, J=1.8, 1.1 Hz, 0H), 7.20 (d, J=2.3 Hz, 1H), 7.06 (d, J=3.0 Hz, 1H), 6.96-6.92 (m, 1H), 6.71 (d, J=4.4 Hz, 1H), 4.31-4.23 (m, 2H), 3.59 (d, J=17.1 Hz, 2H), 2.57 (dd, J=13.7, 3.1 Hz, 6H), 2.42 (d, J=3.1 Hz, 3H), 2.02-1.92 (m, 2H), 1.24 (d, J=3.0 Hz, 6H).

### Example 556: (1s,4s)-4-((4-(2-((2,6-dimethylpyrimidin-4-yl)amino)pyrazolo[1,5-a]pyridin-5-yl)-6-methylpyridin-3-yl)oxy)cyclohexane-1-carbonitrile

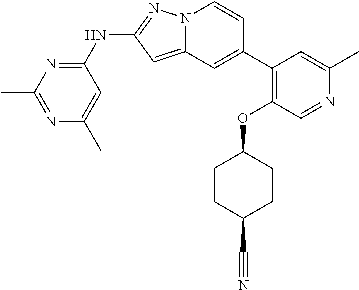

An SFC purification step, similar to that described in Example 5, was performed on Example 552 to yield the title compound. MS (ESI): mass calcd. for C₂₆H₂₇N₇O, 453.2. m/z found, 454.20 [M+H]⁺. ¹H NMR (500 MHz, CDCl₃) δ 8.30 (dt, J=7.1, 0.9 Hz, 1H), 8.20 (s, 1H), 7.54 (dd, J=2.0, 0.9 Hz, 1H), 7.47 (s, 1H), 7.11 (s, 1H), 7.02 (s, 1H), 6.87 (dd, J=7.2, 1.9 Hz, 1H), 6.53 (s, 1H), 4.37-4.30 (m, 1H), 3.42 (s, 1H), 2.56 (s, 3H), 2.49 (s, 3H), 2.40 (s, 3H), 1.99-1.92 (m, 2H), 1.89-1.82 (m, 2H), 1.70-1.54 (m, 2H), 1.20 (s, 2H).

### Example 557: 5-[5-(2-aminoethoxy)-2-methyl-4-pyridyl]-N-(2,6-dimethylpyrimidin-4-yl)pyrazolo[1,5-a]pyridin-2-amine

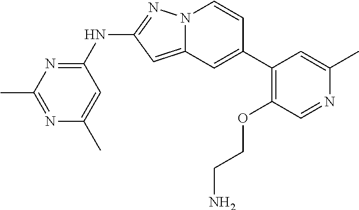

Step A: tert-butyl (2-((4-(2-((2,6-dimethylpyrimidin-4-yl)amino)pyrazolo[1,5-a]pyridin-5-yl)-6-methylpyridin-3-yl)oxy)ethyl)carbamate. The title compound was prepared using a similar method described in Example 203, Step B, and using tert-butyl aziridine-1-carboxylate instead of (3-hydroxytetrahydrofuran-3-yl)methyl 4-methylbenzenesulfonate.

Step B: 5-[5-(2-aminoethoxy)-2-methyl-4-pyridyl]-N-(2,6-dimethylpyrimidin-4-yl)pyrazolo[1,5-a]pyridin-2-amine. A deprotection step with 1:3 TFA:DCM was used on tert-butyl (2-((4-(2-((2,6-dimethylpyrimidin-4-yl)amino)pyrazolo[1,5-a]pyridin-5-yl)-6-methylpyridin-3-yl)oxy)ethyl)carbamate to yield the title compound. MS (ESI): mass calcd. for C₂₁H₂₃N₇O, 389.2. m/z found, 390.1 [M+H]⁺. ¹H NMR (400 MHz, DMSO-d6) δ 10.22 (s, 1H), 8.58 (d, J=7.2 Hz, 1H), 8.34 (s, 1H), 7.84 (s, 1H), 7.36 (s, 1H), 7.05 (dd, J=1.6, 7.2 Hz, 1H), 7.02 (br. s., 1H), 6.87 (br. s., 1H), 4.09 (t, J=5.6 Hz, 2H), 2.89 (t, J=5.6 Hz, 2H), 2.47 (s, 3H), 2.46 (s, 3H), 2.30 (s, 3H).

### Example 558: (1R)-1-cyclopropyl-2-[[4-[2-[(2,6-dimethylpyrimidin-4-yl)amino]pyrazolo[1,5-a]pyridin-5-yl]-6-methyl-3-pyridyl]oxy]ethanol

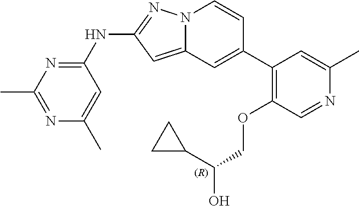

The title compound was prepared using a similar method described in Example 203. Purified by SFC-17 over DAICEL CHIRALCEL OJ-H 250 mm×30 mm×5 μm (eluent: 30% to 30% (v/v) EtOH with 0.1% NH₃—H₂O in supercritical CO₂). MS (ESI): mass calcd. for C₂₄H₂₆N₆O₂, 430.2. m/z found, 431.2 [M+H]⁺. ¹H NMR (400 MHz, CDCl₃) δ 8.34 (d, J=7.2 Hz, 1H), 8.30 (s, 1H), 7.66-7.60 (m, 1H), 7.39 (br. s., 1H), 7.18 (s, 1H), 7.05 (s, 1H), 6.95 (dd, J==2.0, 7.2 Hz, 1H), 6.57 (s, 1H), 4.29-4.20 (m, 1H), 4.15-4.07 (m, 1H), 3.33-3.23 (m, 1H), 2.60 (s, 3H), 2.57 (s, 3H), 2.44 (s, 3H), 2.28 (br. s., 1H), 1.03-0.92 (m, 1H), 0.64-0.51 (m, 2H), 0.46-0.36 (m, 1H), 0.33-0.25 (m, 1H).

### Example 559: (1S)-1-cyclopropyl-2-[[4-[2-[(2,6-dimethylpyrimidin-4-yl)amino]pyrazolo[1,5-a]pyridin-5-yl]-6-methyl-3-pyridyl]oxy]ethanol

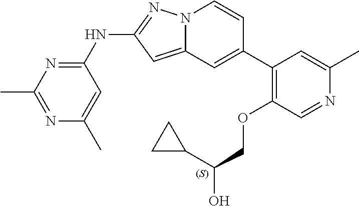

The title compound was prepared using a similar method described in Example 203. Purified by SFC-17 over DAICEL CHIRALCEL OJ-H 250 mm×30 mm×5 μm (eluent: 30% to 30% (v/v) EtOH with 0.1% NH₃—H₂O in supercritical CO₂). MS (ESI): mass calcd. for C₂₄H₂₆N₆O₂, 430.2. m/z found, 431.1 [M+H]⁺. ¹H NMR (400 MHz, CDCl₃) δ 8.34 (d, J=7.2 Hz, 1H), 8.30 (s, 1H), 7.64-7.60 (m, 1H), 7.44 (br. s., 1H), 7.17 (s, 1H), 7.03 (s, 1H), 6.94 (dd, J=2.0, 7.2 Hz, 1H), 6.56 (s, 1H), 4.27-4.19 (m, 1H), 4.13-4.06 (m, 1H), 3.33-3.22 (m, 1H), 2.59 (s, 3H), 2.56 (s, 3H), 2.43 (s, 3H), 2.35 (br. s., 1H), 1.01-0.94 (m, 1H), 0.61-0.53 (m, 2H), 0.44-0.37 (m, 1H), 0.32-0.26 (m, 1H).

### Example 560: 1-[[4-[2-[(2,6-dimethylpyrimidin-4-yl)amino]pyrazolo[1,5-a]pyridin-5-yl]-6-methyl-3-pyridyl]oxymethyl]cyclopropanecarbonitrile

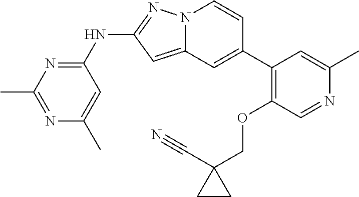

The title compound was prepared using a similar method described in Example 198. MS (ESI): mass calcd. for C₂₄H₂₃N₇O, 425.2. m/z found, 426.20 [M+H]⁺. ¹H NMR (400 MHz, CDCl₃) δ 8.45 (dd, J=7.3, 0.9 Hz, 1H), 8.21 (s, 1H), 7.80 (s, 1H), 7.71 (dd, J=2.0, 0.9 Hz, 1H), 7.22 (s, 1H), 7.10 (s, 1H), 7.02 (dd, J=7.2, 2.0 Hz, 1H), 6.61 (s, 1H), 4.02 (s, 2H), 2.59 (d, J=11.3 Hz, 6H), 2.45 (s, 3H), 1.40-1.32 (m, 2H), 1.07-0.98 (m, 2H).

### Example 561: 2-cyclopropyl-6-[[5-[5-[[(2S)-1,4-dioxan-2-yl]methoxy]-2-methyl-4-pyridyl]pyrazolo[1,5-a]pyridin-2-yl]amino]-4-methyl-pyridazin-3-one

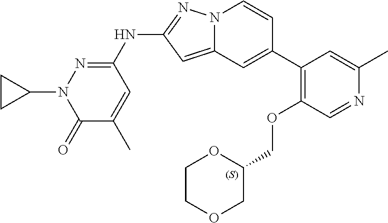

Step A: 2-cyclopropyl-6-((5-(5-fluoro-2-methylpyridin-4-yl)pyrazolo[1,5-a]pyridin-2-yl)amino)-4-methylpyridazin-3(2H)-one. The title compound was prepared as in Intermediate 15, but using 6-chloro-2-cyclopropyl-4-methylpyridazin-3(2H)-one instead of 4-chloro-2,6-dimethylpyrimidine.

Step B: 2-cyclopropyl-6-[[5-[5-[[(2S)-1,4-dioxan-2-yl]methoxy]-2-methyl-4-pyridyl]pyrazolo[1,5-a]pyridin-2-yl]amino]-4-methyl-pyridazin-3-one. The title compound was prepared as in Example 198, and using (s)-(1,4-dioxan-2-yl)methanol instead of trans-1,4-cyclohexanediol. MS (ESI): mass calcd. for C₂₆H₂₈N₆O₄, 488.2. m/z found, 489.20 [M+H]⁺. ¹H NMR (500 MHz, CDCl₃) δ 8.41 (d, J=1.9 Hz, 1H), 8.31-8.24 (m, 2H), 7.73 (dd, J=2.0, 0.9 Hz, 1H), 7.21 (s, 1H), 7.04 (d, J=1.4 Hz, 1H), 6.94 (dd, J=7.2, 2.0 Hz, 1H), 6.72 (d, J=0.8 Hz, 1H), 4.16-4.04 (m, 2H), 3.99-3.93 (m, 1H), 3.89-3.69 (m, 4H), 3.62 (td, J=11.3, 3.0 Hz, 1H), 3.56-3.47 (m, 1H), 3.14-3.09 (m, 1H), 2.56 (s, 3H), 2.22 (d, J=1.3 Hz, 3H), 1.25 (dt, J=7.2, 4.4 Hz, 2H), 1.06-0.94 (m, 2H).

### Example 562: (4S)-4-[[4-[2-[(2,6-dimethylpyrimidin-4-yl)amino]pyrazolo[1,5-a]pyridin-5-yl]-6-methyl-3-pyridyl]oxymethyl]-1,3-dimethyl-imidazolidin-2-one

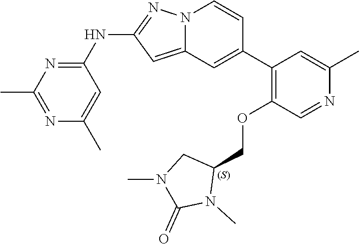

The title compound was prepared using a similar method described in Example 198. MS (ESI): mass calcd. for C₂₅H₂₈N₈O₂, 472.2. m/z found, 473.2 [M+H]⁺. ¹H NMR (400 MHz, DMSO-d6) δ 10.22 (s, 1H), 8.54 (d, J=7.2 Hz, 1H), 8.39 (s, 1H), 7.82 (d, J=0.8 Hz, 1H), 7.40 (s, 1H), 7.04-6.96 (m, 2H), 6.91 (br. s., 1H), 4.35-4.28 (m, 1H), 4.26-4.19 (m, 1H), 3.84-3.75 (m, 1H), 3.46-3.39 (m, 1H), 3.12-3.05 (m, 1H), 2.65 (s, 3H), 2.58 (s, 3H), 2.49-2.45 (m, 6H), 2.30 (s, 3H).

### Example 563: N-[5-[2-cyano-5-[[(3R)-1-(cyanomethyl)pyrrolidin-3-yl]methoxy]-4-pyridyl]pyrazolo[1,5-a]pyridin-2-yl]cyclopropanecarboxamide

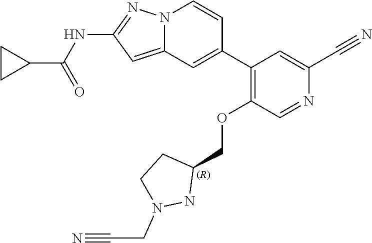

The title compound was prepared using a similar method described in Example 1. MS (ESI): mass calcd. for C₂₄H₂₃N₇O₂, 441.2. m/z found, 442.2 [M+H]⁺. ¹H NMR (400 MHz, DMSO-d6) δ 11.11 (s, 1H), 8.67 (s, 1H), 8.63-8.59 (m, 1H), 8.18 (s, 1H), 7.93-7.91 (m, 1H), 7.11 (dd, J=7.3, 2.0 Hz, 1H), 6.93 (s, 1H), 4.30-4.19 (m, 2H), 3.81 (s, 2H), 2.75-2.54 (m, 4H), 2.49-2.46 (m, 1H), 2.01-1.89 (m, 2H), 1.69-1.52 (m, 1H), 0.88-0.78 (m, 4H).

### Example 564: 5-[2-[(1-methylpyrazol-3-yl)amino]pyrazolo[1,5-a]pyridin-5-yl]-6-[[(2S)-morpholin-2-yl]methoxy]pyridine-3-carbonitrile

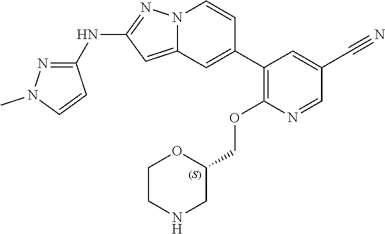

The title compound was prepared using a similar method described in Example 252, and using 5-bromo-6-chloronicotinonitrile instead of 5-chloro-4-iodo-2-(trifluoromethyl)pyridine in Step A and using Brettphos Pd G2 with additional Brettphos instead of BrettPhos Pd G3 in Step C. MS (ESI): mass calcd. for C₂₂H₂₂N₈O₂, 430.2. m/z found, 431.3 [M+H]⁺. ¹H NMR (400 MHz, DMSO-d6) δ 9.07 (s, 1H), 8.69 (d, J=2.2 Hz, 1H), 8.50-8.44 (m, 1H), 8.36 (d, J=2.2 Hz, 1H), 7.79-7.72 (m, 1H), 7.49 (d, J=2.3 Hz, 1H), 6.94 (dd, J=7.2, 2.1 Hz, 1H), 6.42 (s, 1H), 6.06 (d, J=2.2 Hz, 1H), 4.44-4.34 (m, 2H), 3.78-3.70 (m, 5H), 3.49-3.40 (m, 1H), 2.82 (dd, J=12.2, 2.4 Hz, 1H), 2.70-2.61 (m, 2H), 2.56-2.52 (m, 1H).

### Example 565: N-[5-[2-cyano-5-[[(1R,5S)-9-methyl-3-oxa-9-azabicyclo[3.3.1]nonan-7-yl]amino]-4-pyridyl]pyrazolo[1,5-a]pyridin-2-yl]cyclopropanecarboxamide

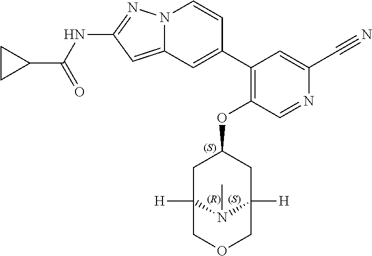

The title compound was prepared using a similar method described in Example 1. MS (ESI): mass calcd. for C₂₅H₂₇N₇O₂, 457.2. m/z found, 458.3 [M+H]⁺. ¹H NMR (400 MHz, DMSO-d6) δ 11.08 (s, 1H), 8.58 (dt, J=7.2, 0.9 Hz, 1H), 8.24 (s, 1H), 7.70 (dd, J=2.1, 0.9 Hz, 1H), 7.65 (s, 1H), 7.20 (d, J=10.7 Hz, 1H), 6.91-6.82 (m, 2H), 4.31-4.15 (m, 1H), 3.60 (dt, J=10.6, 2.2 Hz, 2H), 3.36 (d, J=10.9 Hz, 2H), 2.54-2.51 (m, 2H), 2.42-2.28 (m, 5H), 2.00-1.85 (m, 1H), 1.38 (d, J=14.7 Hz, 2H), 0.89-0.74 (m, 4H).

### Example 566: 3-[2-[(1-methylpyrazol-3-yl)amino]pyrazolo[1,5-a]pyridin-5-yl]-4-[[(2R)-morpholin-2-yl]methoxy]benzonitrile

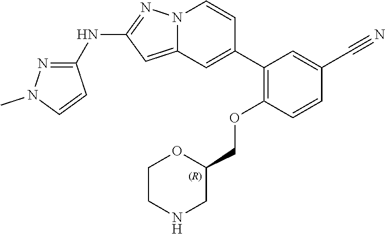

The title compound was prepared using a similar method described in Example 116, Steps A-B, and using (R)-tert-butyl 2-((2-(2-aminopyrazolo[1,5-a]pyridin-5-yl)-4-cyanophenoxy)methyl)morpholine-4-carboxylate instead of Intermediate 9, and using 3-bromo-1-methyl-1H-pyrazole instead of 4-bromo-1-(difluoromethyl)-1H-pyrazole. MS (ESI): mass calcd. for C₂₃H₂₃N₇O₂, 429.2. m/z found, 430.2 [M+H]⁺. ¹H NMR (400 MHz, DMSO-d6) δ 9.06 (s, 1H), 8.43 (d, J=7.2 Hz, 1H), 7.89 (d, J=1.6 Hz, 1H), 7.84 (dd, J=1.6, 8.8 Hz, 1H), 7.68-7.62 (m, 1H), 7.49 (d, J=1.6 Hz, 1H), 7.31 (d, J=8.8 Hz, 1H), 6.87 (dd, J=1.6, 7.2 Hz, 1H), 6.39 (s, 1H), 6.06 (d, J=2.0 Hz, 1H), 4.18-4.06 (m, 2H), 3.78-3.66 (m, 5H), 3.49-3.45 (m, 1H), 2.85-2.76 (m, 1H), 2.70-2.58 (m, 3H) 1H NMR (400 MHz, CD3OD) δ 8.29 (d, J=6.8 Hz, 1H), 7.76 (d, J=2.0 Hz, 1H), 7.71 (dd, J=2.0, 8.4 Hz, 1H), 7.56 (d, J=1.2 Hz, 1H), 7.42 (d, J=2.4 Hz, 1H), 7.22 (d, J=8.8 Hz, 1H), 6.87 (dd, J=2.0, 7.2 Hz, 1H), 6.31 (s, 1H), 6.16 (d, J=2.4 Hz, 1H), 4.20-4.04 (m, 2H), 3.93-3.85 (m, 1H), 3.84-3.75 (m, 4H), 3.67-3.57 (m, 1H), 2.91-2.85 (m, 1H), 2.80-2.73 (m, 2H), 2.71-2.63 (m, 1H)

### Example 567: 5-[2-methyl-5-(1-methylazetidin-3-yl)oxy-4-pyridyl]-N-(2-pyridyl)pyrazolo[1,5-a]pyridin-2-amine

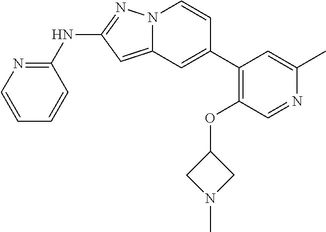 [SMILES: Cc1cc(-c2ccn3nc(Nc4ccccn4)cc3c2)c(OC2CN(C)C2)cn1]

The title compound was prepared using a similar method described in Example 162, Step B, and using 5-(2-methyl-5-((1-methylazetidin-3-yl)oxy)pyridin-4-yl)pyrazolo[1,5-a]pyridin-2-amine instead of 5-(5-((1-amino-3,3-difluorocyclobutyl)methoxy)-2-methylpyridin-4-yl)pyrazolo[1,5-a]pyridin-2-amine and using 2-bromopyridine instead of 3-bromo-1-methyl-1H-pyrazole. MS (ESI): mass calcd. for C₂₂H₂₂N₆O, 386.2. m/z found, 387.2 [M+H]⁺. ¹H NMR (400 MHz, DMSO-d6) δ 9.82 (s, 1H), 8.63-8.48 (m, 1H), 8.22 (s, 1H), 8.06 (s, 1H), 7.78 (s, 1H), 7.69-7.58 (m, 1H), 7.36 (s, 1H), 7.34-7.27 (m, 1H), 7.04-6.93 (m, 1H), 6.90 (s, 1H), 6.84-6.77 (m, 1H), 4.96-4.81 (m, 1H), 3.87-3.59 (m, 2H), 3.12-2.91 (m, 2H), 2.46 (s, 3H), 2.28 (s, 3H).

### Example 568: 5-[2-methyl-5-(1-methylazetidin-3-yl)oxy-4-pyridyl]-N-(1-methylpyrazol-4-yl)pyrazolo[1,5-a]pyridin-2-amine

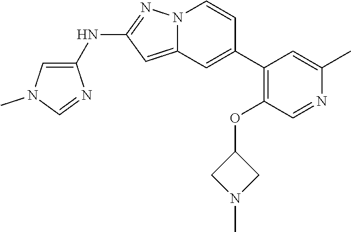

The title compound was prepared using a similar method described in Example 567 and using 4-bromo-1-methyl-1H-pyrazole instead of 2-bromopyridine. MS (ESI): mass calcd. for C₂₁H₂₃N₇O, 389.2. m/z found, 390.2 [M+H]⁺. ¹H NMR (400 MHz, DMSO-d6) δ 8.49-8.42 (m, 2H), 8.04 (s, 1H), 7.77 (s, 1H), 7.60 (d, J=1.2 Hz, 1H), 7.38 (d, J=0.4 Hz, 1H), 7.34 (s, 1H), 6.84 (dd, J=2.0, 7.2 Hz, 1H), 5.95 (s, 1H), 4.97-4.77 (m, 1H), 3.81 (s, 3H), 3.75-3.67 (m, 2H), 3.03-2.93 (m, 2H), 2.45 (s, 3H), 2.27 (s, 3H).

### Example 569: (1*R, 3*R)—N-[5-[5-(3-hydroxycyclopentoxy)-2-methyl-4-pyridyl]pyrazolo[1,5-a]pyridin-2-yl]cyclopropanecarboxamide

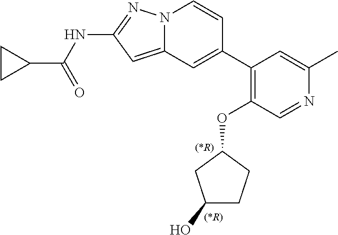 [SMILES: Cc1cc(-c2ccn3nc(NC(=O)C4CC4)cc3c2)c(O[C@@H]2CC[C@@H](O)C2)cn1]

The title compound was prepared using methods analogous to those described in Example 136. Purified by chiral SFC over DAICEL CHIRALPAK AD (250 mm×30 mm×10 um) (eluent: supercritical CO₂ in EtOH (0.1% v/v ammonia) 50/50, v/v). MS (ESI): mass calcd. for C₂₂H₂₄N₄O₃, 392.2. m/z found, 393.2 [M+H]⁺. ¹H NMR (400 MHz, DMSO-d6) δ 11.10 (s, 1H), 8.56 (d, J=7.2 Hz, 1H), 8.27 (s, 1H), 7.77 (d, J=1.2 Hz, 1H), 7.34 (s, 1H), 7.00 (dd, J=2.0, 7.2 Hz, 1H), 6.89 (s, 1H), 5.04-4.95 (m, 1H), 4.60 (d, J=3.6 Hz, 1H), 4.24-4.16 (m, 1H), 2.45 (s, 3H), 2.18-2.06 (m, 1H), 1.98-1.87 (m, 3H), 1.86-1.75 (m, 1H), 1.72-1.63 (m, 1H), 1.54-1.44 (m, 1H), 0.88-0.77 (m, 4H).

### Example 570: N-[5-[5-[(2R)-2-amino-3,3,3-trifluoro-propoxy]-2-methyl-4-pyridyl]pyrazolo[1,5-a]pyridin-2-yl]cyclopropanecarboxamide

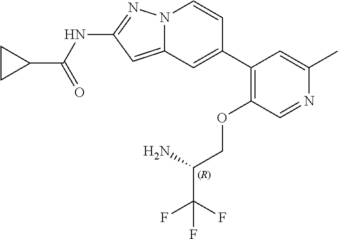

The title compound was prepared using a similar method described in Example 188. MS (ESI): mass calcd. for C₂₀H₂₀F₃N₅O₂, 419.2. m/z found, 420.2 [M+H]⁺. ¹H NMR (400 MHz, CDCl₃) δ 8.45 (s, 1H), 8.28 (d, J=7.6 Hz, 1H), 8.26 (s, 1H), 7.64 (s, 1H), 7.19 (s, 1H), 7.02 (s, 1H), 6.92 (d, J=7.2 Hz, 1H), 4.25 (d, J=4.4 Hz, 2H), 3.62-3.52 (m, 1H), 2.57 (s, 3H), 1.62-1.52 (m, 3H), 1.19-1.11 (m, 2H), 0.95-0.88 (m, 2H). ¹⁹F NMR (376 MHz, CDCl₃) δ −76.13 (d, J=7.1 Hz, 3F).

### Example 571: (3RS, 4RS)—N-[5-[5-[(4-methoxy-1-methyl-pyrrolidin-3-yl)methoxy]-2-methyl-4-pyridyl]pyrazolo[1,5-a]pyridin-2-yl]cyclopropanecarboxamide

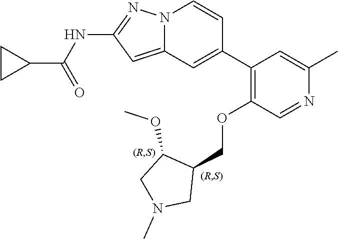 [SMILES: CO[C@H]1CN(C)C[C@@H]1COc1cnc(C)cc1-c1ccn2nc(NC(=O)C3CC3)cc2c1]

The title compound was prepared using a similar method described in Example 248. MS (ESI): mass calcd. for C₂₄H₂₉N₅O₃, 435.2. m/z found, 436.2 [M+H]⁺. ¹H NMR (500 MHz, CD₃OD) d 8.45-8.39 (m, 1H), 8.24 (s, 1H), 7.77-7.73 (m, 1H), 7.36 (s, 1H), 7.01 (dd, J=7.2, 2.0 Hz, 1H), 6.90 (s, 1H), 4.18-4.09 (m, 2H), 3.74-3.70 (m, 1H), 3.16 (s, 3H), 2.96-2.90 (m, 1H), 2.74 (dd, J=10.6, 2.5 Hz, 1H), 2.58-2.54 (m, 1H), 2.53 (s, 3H), 2.47-2.42 (m, 1H), 2.25 (s, 3H), 2.23-2.18 (m, 1H), 1.92-1.83 (m, 1H), 1.04-0.98 (m, 2H), 0.93-0.85 (m, 2H).

### Example 572: N-[5-[2-methyl-5-[[(2S)-1-methylpyrrolidin-2-yl]methoxy]-4-pyridyl]pyrazolo[1,5-a]pyridin-2-yl]cyclopropanecarboxamide

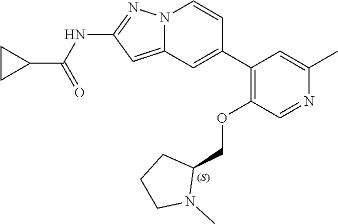

The title compound was prepared using a similar method described in Example 136. MS (ESI): mass calcd. for C₂₃H₂₇N₅O₂, 405.2. m/z found, 406.2 [M+H]⁺. ¹H NMR (400 MHz, CDCl₃) δ 9.30 (s, 1H), 8.21 (d, J=8.2 Hz, 2H), 7.67-7.62 (m, 1H), 7.09 (s, 1H), 6.96 (s, 1H), 6.88 (dd, J=7.2, 1.9 Hz, 1H), 4.03 (dd, J=9.1, 5.2 Hz, 1H), 3.88 (dd, J=9.0, 5.9 Hz, 1H), 2.98 (ddd, J=9.1, 6.7, 2.2 Hz, 1H), 2.59-2.48 (m, 1H), 2.47 (s, 3H), 2.29 (s, 3H), 2.18 (td, J=9.4, 7.2 Hz, 1H), 1.96-1.82 (m, 1H), 1.78-1.46 (m, 4H), 1.17-1.04 (m, 2H), 0.80 (dq, J=7.4, 4.0 Hz, 2H).

### Example 573: N-[5-[5-[[(2S)-1-(2-fluoroethyl)azetidin-2-yl]methoxy]-2-methyl-4-pyridyl]pyrazolo[1,5-a]pyridin-2-yl]cyclopropanecarboxamide

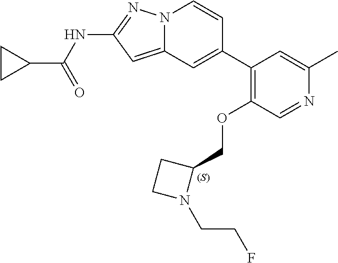

The title compound was prepared using methods analogous to those described in Example 457, Step B. MS (ESI): mass calcd. for C₂₃H₂₆FN₅O₂, 423.2. m/z found, 424.3 [M+H]t ¹H NMR (400 MHz, CDCl₃) δ 8.31 (s, 1H), 8.28-8.23 (m, 2H), 7.69 (s, 1H), 7.15 (s, 1H), 6.99 (s, 1H), 6.95 (dd, J=1.6, 7.2 Hz, 1H), 4.44-4.20 (m, 2H), 4.16-4.04 (m, 2H), 3.57-3.45 (m, 2H), 3.02-2.90 (m, 1H), 2.89-2.73 (m, 1H), 2.72-2.57 (m, 1H), 2.55 (s, 3H), 2.13-2.02 (m, 2H), 1.61-1.51 (m, 1H), 1.19-1.09 (m, 2H), 0.97-0.85 (m, 2H). ¹⁹F NMR (376 MHz, CDCl₃) δ 221.47-−221.92 (m, 1F).

### Example 574: N-[5-[5-[[(2R)-1-cyclopropylazetidin-2-yl]methoxy]-2-methyl-4-pyridyl]pyrazolo[1,5-a]pyridin-2-yl]cyclopropanecarboxamide

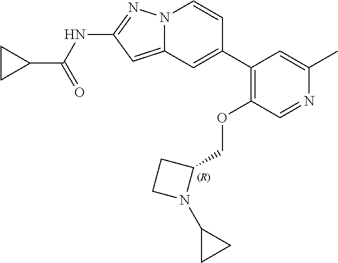

The title compound was prepared using methods analogous to those described in Example 7 and using (R)—N-(5-(5-(azetidin-2-ylmethoxy)-2-methylpyridin-4-yl)pyrazolo[1,5-a]pyridin-2-yl)cyclopropanecarboxamide instead of (S)—N-(5-(2-cyano-5-(pyrrolidin-3-yloxy)pyridin-4-yl)pyrazolo[1,5-a]pyridin-2-yl)cyclopropanecarboxamide. MS (ESI): mass calcd. for C₂₄H₂₇N₅O₂, 417.2. m/z found, 418.2 [M+H]⁺. ¹H NMR (400 MHz, DMSO-d6) δ 11.09 (s, 1H), 8.57 (d, J=7.6 Hz, 1H), 8.32 (s, 1H), 7.95 (d, J=0.8 Hz, 1H), 7.36 (s, 1H), 7.11 (dd, J=1.6, 7.2 Hz, 1H), 6.86 (s, 1H), 4.16-4.09 (m, 1H), 4.08-4.02 (m, 1H), 3.65-3.54 (m, 1H), 3.24-3.15 (m, 1H), 3.04-2.94 (m, 1H), 2.46 (s, 3H), 2.02-1.81 (m, 4H), 0.89-0.76 (m, 4H), 0.26-0.17 (m, 1H), 0.16-0.06 (m, 3H)

### Example 575: N-[5-[2-methyl-5-[[(2S)-1-(trideuteriomethyl)azetidin-2-yl]methoxy]-4-pyridyl]pyrazolo[1,5-a]pyridin-2-yl]cyclopropanecarboxamide

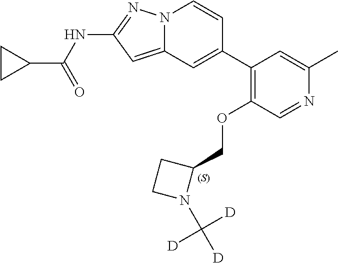

The title compound was prepared using the Suzuki coupling method analogous to that described in Example 329, Step D, and using (S)-4-iodo-2-methyl-5-((1-methyl-d3-azetidin-2-yl)methoxy)pyridine and Intermediate 3 instead of 3-((6-(difluoromethoxy)-4-iodopyridin-3-yl)oxy)-2,2-dimethylpropanenitrile and Intermediate 22. MS (ESI): mass calcd. for C₂₂H₂₂D₃N₅O₂, 394.2. m/z found, 395.1 [M+H]⁺. ¹H NMR (400 MHz, CDCl₃) δ 8.54-8.45 (m, 1H), 8.30-8.20 (m, 2H), 7.71 (d, J=0.8 Hz, 1H), 7.16 (s, 1H), 6.99 (s, 1H), 6.96 (dd, J=2.0, 7.6 Hz, 1H), 4.10-4.04 (m, 2H), 3.46-3.29 (m, 2H), 2.87-2.78 (m, 1H), 2.55 (s, 3H), 2.07-1.96 (m, 2H), 1.62-1.52 (m, 1H), 1.19-1.11 (m, 2H), 0.95-0.86 (m, 2H).

### Example 576: N-[5-[2-methyl-5-[[(2R)-1-(trideuteriomethyl)azetidin-2-yl]methoxy]-4-pyridyl]pyrazolo[1,5-a]pyridin-2-yl]cyclopropanecarboxamide

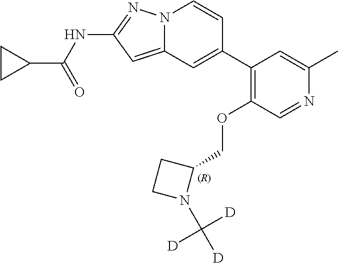

The title compound was prepared using the Suzuki coupling method analogous to that described in Example 575 and using (R)-4-iodo-2-methyl-5-((1-methyl-d3-azetidin-2-yl)methoxy)pyridine instead of (S)-4-iodo-2-methyl-5-((1-methyl-d3-azetidin-2-yl)methoxy)pyridine. MS (ESI): mass calcd. for C₂₂H₂₂D₃N₅O₂, 394.2. m/z found, 395.1 [M+H]⁺. ¹H NMR (400 MHz, CDCl₃) δ 8.51 (s, 1H), 8.28-8.23 (m, 2H), 7.71 (d, J=1.2 Hz, 1H), 7.16 (s, 1H), 6.99 (s, 1H), 6.96 (dd, J=1.6, 7.2 Hz, 1H), 4.12-4.05 (m, 2H), 3.46-3.33 (m, 2H), 2.88-2.80 (m, 1H), 2.55 (s, 3H), 2.08-1.98 (m, 2H), 1.62-1.54 (m, 1H), 1.17-1.12 (m, 2H), 0.94-0.87 (m, 2H).

### Example 577: (*R)—N-(2,6-dimethylpyrimidin-4-yl)-5-[5-(5,5-dimethyltetrahydrofuran-3-yl)oxy-2-methyl-4-pyridyl]pyrazolo[1,5-a]pyridin-2-amine

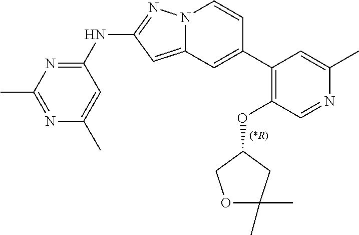

The title compound was prepared using a similar method described in Example 198. Purified by SFC over DAICEL CHIRALPAK AD 250 mm×30 mm×10 μm (eluent: 45% to 45% (v/v) EtOH with 0.1% NH₃—H₂O in supercritical CO₂). MS (ESI): mass calcd. for C₂₅H₂₈N₆O₂, 444.2. m/z found, 445.2 [M+H]⁺. ¹H NMR (400 MHz, DMSO-d6) δ 10.19 (s, 1H), 8.58 (d, J=7.2 Hz, 1H), 8.27 (s, 1H), 7.80 (d, J=1.2 Hz, 1H), 7.36 (s, 1H), 7.04-6.98 (m, 2H), 6.85 (s, 1H), 5.22-5.15 (m, 1H), 4.04 (dd, J=4.8, 10.4 Hz, 1H), 3.85 (d, J=10.4 Hz, 1H), 2.46 (s, 3H), 2.46 (s, 3H), 2.30 (s, 3H), 2.15 (dd, J=6.8, 13.6 Hz, 1H), 1.89 (d, J=14.0 Hz, 1H), 1.19 (s, 3H), 1.18 (s, 3H).

### Example 578: (*S)—N-(2,6-dimethylpyrimidin-4-yl)-5-[5-(2,2-dimethyltetrahydropyran-4-yl)oxy-2-methyl-4-pyridyl]pyrazolo[1,5-a]pyridin-2-amine

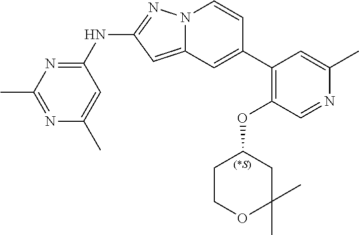

The title compound was prepared using a similar method described in Example 198. Purified by SFC over DAICEL CHIRALCEL OD-H 250 mm×30 mm×5 μm (eluent: 45% to 45% (v/v) IPA with 0.1% NH₃—H₂O in supercritical CO₂). MS (ESI): mass calcd. for C₂₆H₃₀N₆O₂, 458.2. m/z found, 459.2 [M+H]⁺. ¹H NMR (500 MHz, CDCl₃) δ 8.37 (d, J=7.0 Hz, 1H), 8.28 (s, 1H), 7.64-7.60 (m, 1H), 7.47 (s, 1H), 7.19 (s, 1H), 7.07 (s, 1H), 6.95 (dd, J=1.5, 7.0 Hz, 1H), 6.59 (s, 1H), 4.58-4.49 (m, 1H), 3.85-3.78 (m, 1H), 3.67-3.59 (m, 1H), 2.61 (s, 3H), 2.56 (s, 3H), 2.45 (s, 3H), 2.01-1.92 (m, 2H), 1.64-1.58 (m, 1H), 1.55-1.48 (m, 1H), 1.22 (s, 3H), 1.18 (s, 3H).

### Example 579: N-[3-[[4-[2-[(2,6-dimethylpyrimidin-4-yl)amino]pyrazolo[1,5-a]pyridin-5-yl]-6-methyl-3-pyridyl]oxy]cyclobutyl]methanesulfonamide

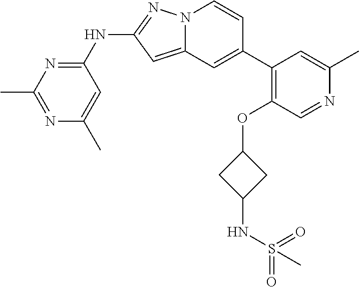

The title compound was prepared using a similar method described in Example 198. MS (ESI): mass calcd. for C₂₄H₂₇N₇O₃S, 493.2. m/z found, 494.20 [M+H]⁺. ¹H NMR (500 MHz, CDCl₃) δ 8.36-8.28 (m, 1H), 8.01 (s, 1H), 7.80 (s, 1H), 7.57-7.51 (m, 1H), 7.11 (d, J=6.6 Hz, 1H), 6.97 (d, J=17.4 Hz, 1H), 6.84 (dd, J=7.2, 2.0 Hz, 1H), 6.57 (d, J=12.8 Hz, 1H), 5.13 (s, 1H), 4.41 (p, J=6.9 Hz, 1H), 3.64 (p, J=8.0 Hz, 1H), 3.01-2.91 (m, 2H), 2.86 (d, J=11.2 Hz, 3H), 2.51 (d, J=31.9 Hz, 6H), 2.37 (s, 3H), 2.14-2.04 (m, 2H).

### Example 580: N-[3-[[4-[2-[(2,6-dimethylpyrimidin-4-yl)amino]pyrazolo[1,5-a]pyridin-5-yl]-6-methyl-3-pyridyl]oxy]cyclobutyl]methanesulfonamide

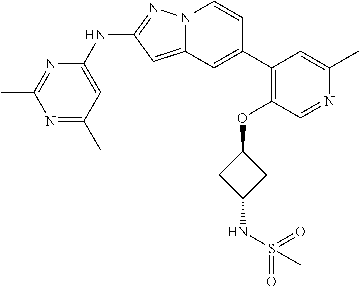

The title compound was obtained by an SFC chiral purification (similar to that described in Example 5) of Example 579. MS (ESI): mass calcd. for C₂₄H₂₇N₇O₃S, 493.2. m/z found, 494.1 [M+H]⁺. ¹H NMR (400 MHz, CDCl₃) δ 8.38 (d, J=7.2 Hz, 1H), 8.00 (s, 1H), 7.62 (d, J=1.2 Hz, 1H), 7.48 (s, 1H), 7.20 (s, 1H), 7.07 (s, 1H), 6.93 (dd, J=2.0, 7.2 Hz, 1H), 6.62 (s, 1H), 4.93-4.86 (m, 1H), 4.81 (br. s., 1H), 4.26-4.14 (m, 1H), 2.93 (s, 3H), 2.70-2.62 (m, 2H), 2.61 (s, 3H), 2.56 (s, 3H), 2.52-2.47 (m, 2H), 2.46 (s, 3H).

### Example 581: (1r,4r)-4-((4-(2-((2,6-dimethylpyrimidin-4-yl)amino)pyrazolo[1,5-a]pyridin-5-yl)-6-methylpyridin-3-yl)oxy)cyclohexane-1-carbonitrile

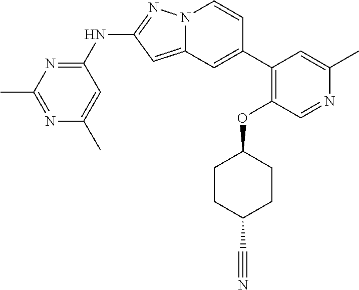

An SFC purification step, similar to that described in Example 5, was performed on Example 552 to yield the title compound. MS (ESI): mass calcd. for C₂₆H₂₇N₇O, 453.2. m/z found, 454.2 [M+H]⁺. ¹H NMR (500 MHz, CDCl₃) δ 8.43 (dt, J=7.3, 1.0 Hz, 1H), 8.27 (s, 1H), 7.69 (s, 1H), 7.64 (dd, J=2.0, 0.9 Hz, 1H), 7.21 (s, 1H), 7.13 (s, 1H), 6.97 (dd, J=7.2, 1.9 Hz, 1H), 6.63 (s, 1H), 4.36 (dt, J=6.5, 3.6 Hz, 1H), 2.68 (dt, J=8.2, 4.0 Hz, 1H), 2.63 (s, 3H), 2.58 (s, 3H), 2.48 (s, 3H), 2.00-1.92 (m, 4H), 1.85-1.72 (m, 4H).

### Example 582: (3*R, 6*R)-6-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol

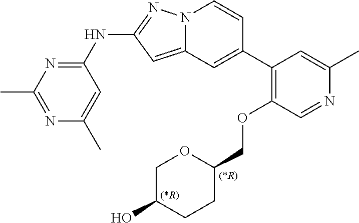

The title compound was prepared using a similar method described in Example 203. Purified by SFC over DAICEL CHIRALPAK AD 250 mm×30 mm, 10 μm (eluent: 40% to 40% (v/v) supercritical CO₂ in IPA and H₂O with 0.1% NH₃). MS (ESI): mass calcd. for C₂₅H₂₈N₆O₃, 460.2. m/z found, 461.1 [M+H]⁺. ¹H NMR (400 MHz, DMSO-d6) δ 10.21 (s, 1H), 8.57 (d, J=7.2 Hz, 1H), 8.35 (s, 1H), 7.93 (s, 1H), 7.38 (s, 1H), 7.14 (dd, J=1.6, 7.6 Hz, 1H), 7.01 (s, 1H), 6.88 (s, 1H), 4.61 (s, 1H), 4.17-4.06 (m, 2H), 3.82-3.71 (m, 1H), 3.70-3.62 (m, 1H), 3.61-3.56 (m, 1H), 3.53-3.44 (m, 1H), 2.46 (s, 3H), 2.46 (s, 3H), 2.30 (s, 3H), 1.87-1.58 (m, 3H), 1.46-1.34 (m, 1H).

### Example 583: N-[5-(2-cyano-5-ethoxy-4-pyridyl)pyrazolo[1,5-a]pyridin-2-yl]cyclopropanecarboxamide

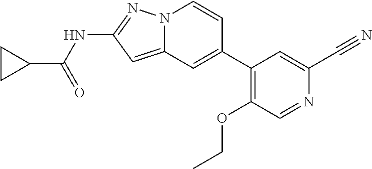 [SMILES: CCOc1cnc(C#N)cc1-c1ccn2nc(NC(=O)C3CC3)cc2c1]

The title compound was prepared using a similar method described in Example 1. MS (ESI): mass calcd. for C₁₉H₁₇N₅O₂, 347.1. m/z found, 348.2 [M+H]⁺. ¹H NMR (400 MHz, DMSO-d6) δ 11.11 (s, 1H), 8.65 (s, 1H), 8.62-8.58 (m, 1H), 8.17 (s, 1H), 7.94-7.92 (m, 1H), 7.11 (dd, J=7.3, 2.0 Hz, 1H), 6.94-6.91 (m, 1H), 4.37 (q, J=7.0 Hz, 2H), 1.99-1.89 (m, 1H), 1.37 (t, J=6.9 Hz, 3H), 0.86-0.79 (m, 4H).

### Example 584: N-[5-[2-cyano-5-(cyclopropylmethoxy)-4-pyridyl]pyrazolo[1,5-a]pyridin-2-yl]cyclopropanecarboxamide

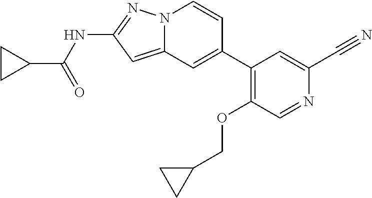 [SMILES: N#Cc1cc(-c2ccn3nc(NC(=O)C4CC4)cc3c2)c(OCC2CC2)cn1]

The title compound was prepared using a similar method described in Example 1. MS (ESI): mass calcd. for C₂₁H₁₉N₅O₂, 373.2. m/z found, 374.2 [M+H]⁺. ¹H NMR (400 MHz, CDCl₃) δ 8.41 (s, 1H), 8.34-8.28 (m, 2H), 7.75 (dd, J=2.0, 0.9 Hz, 1H), 7.73 (s, 1H), 7.06 (s, 1H), 6.93 (dd, J=7.3, 2.0 Hz, 1H), 4.08 (d, J=6.9 Hz, 2H), 1.65-1.53 (m, 1H), 1.33-1.21 (m, 1H), 1.18-1.12 (m, 2H), 0.95-0.88 (m, 2H), 0.71-0.63 (m, 2H), 0.41-0.32 (m, 2H).

### Example 585: N-[5-[2-cyano-5-[[(3R)-3-piperidyl]methoxy]-4-pyridyl]pyrazolo[1,5-a]pyridin-2-yl]cyclopropanecarboxamide

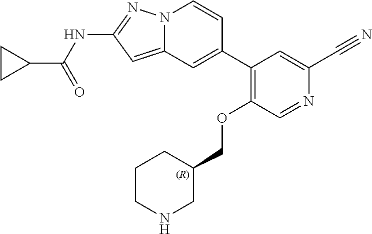

The title compound was prepared using a similar method described in Example 1. MS (ESI): mass calcd. for C₂₃H₂₄N₆O₂, 416.2. m/z found, 417.2 [M+H]⁺. ¹H NMR (400 MHz, DMSO-d6) δ 11.16 (s, 1H), 8.67 (s, 1H), 8.64 (d, J=7.2 Hz, 1H), 8.19 (s, 1H), 7.91 (s, 1H), 7.09 (dd, J=1.6, 7.2 Hz, 1H), 6.94 (s, 1H), 4.27-4.17 (m, 2H), 3.15-3.10 (m, 1H), 3.07-3.01 (m, 1H), 2.63-2.53 (m, 2H), 2.16-2.02 (m, 1H), 1.99-1.89 (m, 1H), 1.84-1.65 (m, 2H), 1.57-1.44 (m, 1H), 1.35-1.20 (m, 1H), 0.86-0.80 (m, 4H).

### Example 586: N-(5-(5-(((1r,4r)-4-aminocyclohexyl)methoxy)-2-cyanopyridin-4-yl)pyrazolo[1,5-a]pyridin-2-yl)cyclopropanecarboxamide

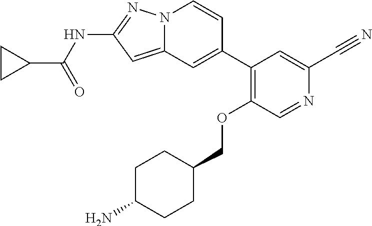 [SMILES: N#Cc1cc(-c2ccn3nc(NC(=O)C4CC4)cc3c2)c(OC[C@H]2CC[C@H](N)CC2)cn1]

The title compound was prepared using methods analogous to those described in Example 8. MS (ESI): mass calcd. for C₂₄H₂₆N₆O₂, 430.2. m/z found, 431.3 [M+H]⁺. ¹H NMR (400 MHz, DMSO-d6) δ 11.16 (s, 1H), 8.68-8.63 (m, 2H), 8.19 (s, 1H), 7.92 (s, 1H), 7.09 (dd, J=1.6, 7.2 Hz, 1H), 6.92 (s, 1H), 4.15 (d, J=6.0 Hz, 2H), 2.92-2.80 (m, 1H), 2.00-1.89 (m, 3H), 1.88-1.69 (m, 3H), 1.33-1.23 (m, 2H), 1.18-1.07 (m, 2H), 0.86-0.80 (m, 4H).

### Example 587: N-[5-[5-(6-azaspiro[3.3]heptan-2-yloxy)-2-cyano-4-pyridyl]pyrazolo[1,5-a]pyridin-2-yl]cyclopropanecarboxamide

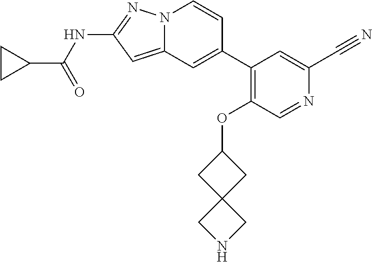 [SMILES: N#Cc1cc(-c2ccn3nc(NC(=O)C4CC4)cc3c2)c(OC2CC3(CNC3)C2)cn1]

The title compound was prepared using methods analogous to those described in Example 6. MS (ESI): mass calcd. for C₂₃H₂₂N₆O₂, 414.2. m/z found, 415.2 [M+H]⁺. ¹H NMR (400 MHz, DMSO-d6) δ 11.12 (s, 1H), 8.61 (d, J=7.3 Hz, 1H), 8.45 (s, 1H), 8.17 (s, 1H), 7.91 (d, J=1.9 Hz, 1H), 7.09 (dd, J=7.3, 2.0 Hz, 1H), 6.93 (s, 1H), 4.94 (t, J=6.7 Hz, 1H), 3.47 (d, J=35.5 Hz, 4H), 2.74 (ddd, J=10.1, 6.8, 3.2 Hz, 2H), 2.19 (t, J=9.8 Hz, 2H), 1.93 (td, J=7.6, 3.9 Hz, 1H), 0.91-0.73 (m, 4H).

### Example 588: N-[5-[2-cyano-5-[[(3R)-1-(2,2,2-trifluoroethyl)pyrrolidin-3-yl]methoxy]-4-pyridyl]pyrazolo[1,5-a]pyridin-2-yl]cyclopropanecarboxamide

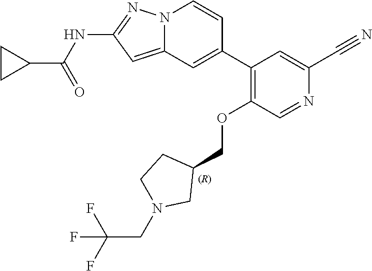

The title compound was prepared using methods analogous to those described in Example 5. MS (ESI): mass calcd. for C₂₄H₂₃F₃N₆O₂, 484.2. m/z found, 485.1 [M+H]⁺. ¹H NMR (400 MHz, DMSO-d6) δ 11.11 (s, 1H), 8.67 (s, 1H), 8.62-8.57 (m, 1H), 8.18 (s, 1H), 7.93-7.89 (m, 1H), 7.10 (dd, J=7.3, 2.0 Hz, 1H), 6.96-6.89 (m, 1H), 4.30-4.18 (m, 2H), 3.28-3.16 (m, 2H), 2.84 (t, J=7.9 Hz, 1H), 2.74-2.65 (m, 2H), 2.64-2.52 (m, 2H), 1.99-1.87 (m, 2H), 1.64-1.52 (m, 1H), 0.88-0.77 (m, 4H).

### Example 589: N-[5-[2-cyano-5-[[(3R)-1-cyclopropylpyrrolidin-3-yl]methoxy]-4-pyridyl]pyrazolo[1,5-a]pyridin-2-yl]cyclopropanecarboxamide

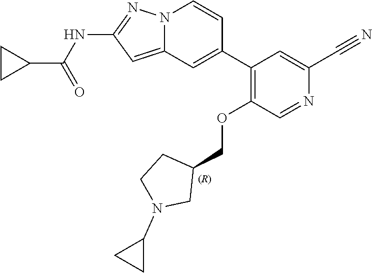

The title compound was prepared using methods analogous to those described in Example 7. MS (ESI): mass calcd. for C₂₅H₂₆N₆O₂, 442.2. m/z found, 443.3 [M+H]⁺. ¹H NMR (400 MHz, DMSO-d6) δ 11.11 (s, 1H), 8.66 (s, 1H), 8.64-8.60 (m, 1H), 8.18 (s, 1H), 7.94-7.90 (m, 1H), 7.10 (dd, J=7.3, 2.0 Hz, 1H), 6.92 (s, 1H), 4.20 (d, J=6.9 Hz, 2H), 2.78-2.71 (m, 1H), 2.63-2.52 (m, 3H), 2.49-2.42 (m, 1H), 1.98-1.82 (m, 2H), 1.61-1.44 (m, 2H), 0.87-0.78 (m, 4H), 0.40-0.30 (m, 2H), 0.29-0.20 (m, 2H).

### Example 590: N-[5-[2-cyano-5-[1-(2,2,2-trifluoroethyl)azetidin-3-yl]oxy-4-pyridyl]pyrazolo[1,5-a]pyridin-2-yl]cyclopropanecarboxamide

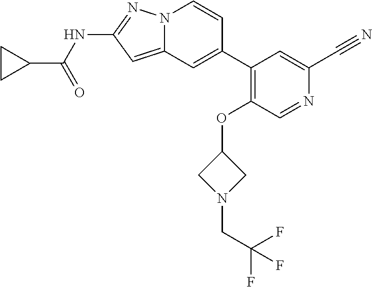 [SMILES: N#Cc1cc(-c2ccn3nc(NC(=O)C4CC4)cc3c2)c(OC2CN(CC(F)(F)F)C2)cn1]

The title compound was prepared using methods analogous to those described in Example 5. MS (ESI): mass calcd. for C₂₂H₁₉F₃N₆O₂, 456.2. m/z found, 457.2 [M+H]⁺. ¹H NMR (400 MHz, DMSO-d6) δ 11.12 (s, 1H), 8.62 (d, J=7.3 Hz, 1H), 8.40 (s, 1H), 8.22 (s, 1H), 7.97 (d, J=1.9 Hz, 1H), 7.14 (dd, J=7.3, 2.1 Hz, 1H), 6.95 (s, 1H), 5.29-5.16 (m, 1H), 3.94 (t, J=7.2 Hz, 2H), 3.45-3.36 (m, 2H), 2.02-1.85 (m, 1H), 0.93-0.72 (m, 4H). 19F NMR (376 MHz, DMSO) d −69.85, −69.88, −69.90.

### Example 591: N-(5-(5-(((1r,3r)-3-aminocyclobutyl)methoxy)-2-cyanopyridin-4-yl)pyrazolo[1,5-a]pyridin-2-yl)cyclopropanecarboxamide

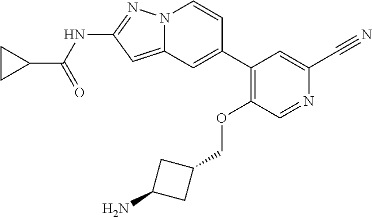 [SMILES: N#Cc1cc(-c2ccn3nc(NC(=O)C4CC4)cc3c2)c(OC[C@H]2C[C@H](N)C2)cn1]

The title compound was prepared using methods analogous to those described in Example 6. MS (ESI): mass calcd. for C₂₂H₂₂N₆O₂, 402.2. m/z found, 403.2 [M+H]⁺. ¹H NMR (400 MHz, DMSO-d6) δ 11.14 (s, 1H), 8.67 (s, 1H), 8.62 (d, J=6.8 Hz, 1H), 8.18 (s, 1H), 7.92 (s, 1H), 7.09 (d, J=6.4 Hz, 1H), 6.91 (s, 1H), 4.32 (d, J=6.4 Hz, 2H), 3.17-3.08 (m, 1H), 2.64-2.53 (m, 1H), 2.20-2.01 (m, 2H), 1.99-190 (m, 1H), 1.87-1.68 (m, 2H), 0.97-0.73 (m, 4H).

### Example 592: N-[5-[2-cyano-5-[[(2R)-morpholin-2-yl]methoxy]-4-pyridyl]pyrazolo[1,5-a]pyridin-2-yl]cyclopropanecarboxamide

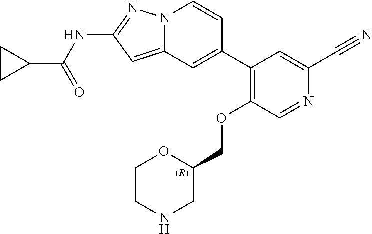

The title compound was prepared using methods analogous to those described in Example 6. MS (ESI): mass calcd. for C₂₂H₂₂N₆O₃, 418.2. m/z found, 419.1 [M+H]⁺. ¹H NMR (400 MHz, DMSO-d6) δ 11.15 (s, 1H), 8.66 (s, 1H), 8.63 (d, J=7.2 Hz, 1H), 8.20 (s, 1H), 8.02 (d, J=1.2 Hz, 1H), 7.16 (dd, J=2.0, 7.2 Hz, 1H), 6.92 (s, 1H), 4.34-4.27 (m, 2H), 3.80-3.69 (m, 2H), 3.54-3.44 (m, 1H), 2.82 (dd, J=1.6, 12.0 Hz, 1H), 2.69-2.53 (m, 3H), 1.98-1.89 (m, 1H), 0.87-0.79 (m, 4H).

### Example 593: N-[5-[2-cyano-5-[[(3S)-3-piperidyl]methoxy]-4-pyridyl]pyrazolo[1,5-a]pyridin-2-yl]cyclopropanecarboxamide

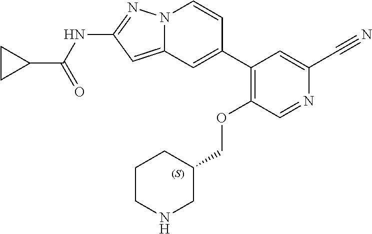

The title compound was prepared using methods analogous to those described in Example 6. MS (ESI): mass calcd. for C₂₃H₂₄N₆O₂, 416.2. m/z found, 417.1 [M+H]⁺. ¹H NMR (400 MHz, DMSO-d6) δ 11.15 (s, 1H), 8.67 (s, 1H), 8.63 (d, J=7.2 Hz, 1H), 8.19 (s, 1H), 7.91 (s, 1H), 7.09 (dd, J=1.6, 7.2 Hz, 1H), 6.94 (s, 1H), 4.27-4.17 (m, 2H), 3.20-2.98 (m, 2H), 2.64-2.54 (m, 2H), 2.17-2.04 (m, 1H), 2.00-1.92 (m, 1H), 1.83-1.66 (m, 2H), 1.57-1.45 (m, 1H), 1.33-1.26 (m, 1H), 0.85-0.81 (m, 4H).

### Example 594: N-[5-[2-cyano-5-[(3R)-pyrrolidin-3-yl]oxy-4-pyridyl]pyrazolo[1,5-a]pyridin-2-yl]cyclopropanecarboxamide

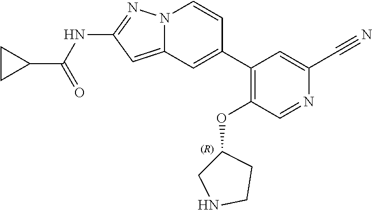

The title compound was prepared using methods analogous to those described in Example 6, except (R)-pyrrolidin-3-ol was used instead of (S)-pyrrolidin-3-ol. MS (ESI): mass calcd. for C₂₁H₂₀N₆O₂, 388.2. m/z found, 389.1 [M+H]⁺. ¹H NMR (400 MHz, DMSO-d6) δ 11.14 (s, 1H), 8.64 (s, 1H), 8.59 (d, J=6.8 Hz, 1H), 8.18 (s, 1H), 7.93 (s, 1H), 7.10 (dd, J=1.6, 7.2 Hz, 1H), 6.93 (s, 1H), 5.28-5.22 (m, 1H), 3.15-3.07 (m, 1H), 2.99-2.86 (m, 2H), 2.84-2.76 (m, 1H), 2.16-2.03 (m, 1H), 1.99-1.89 (m, 1H), 1.88-1.79 (m, 1H), 0.86-0.79 (m, 4H)

### Example 595: (1*S, 4*S, 5*R)—N-[5-[2-cyano-5-[(2-methyl-2-azabicyclo[2.2.1]heptan-5-yl)oxy]-4-pyridyl]pyrazolo[1,5-a]pyridin-2-yl]cyclopropanecarboxamide

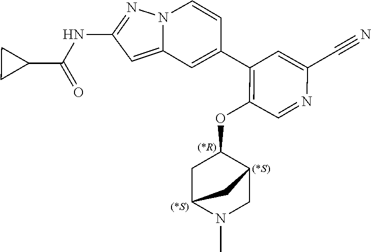 [SMILES: CN1C[C@@H]2C[C@H]1C[C@H]2Oc1cnc(C#N)cc1-c1ccn2nc(NC(=O)C3CC3)cc2c1]

The title compound was prepared using the reductive amination method analogous to that described in Example 164, followed by the SFC purification step described in Example 5. MS (ESI): mass calcd. for C₂₄H₂₄N₆O₂, 428.2. m/z found, 429.2 [M+H]⁺. ¹H NMR (400 MHz, DMSO-d6) δ 11.15 (s, 1H), 8.68 (s, 1H), 8.61 (d, J=7.2 Hz, 1H), 8.19 (s, 1H), 7.89 (s, 1H), 7.10-7.03 (m, 1H), 6.93 (s, 1H), 4.93-4.76 (m, 1H), 3.55-3.51 (m, 1H), 3.00-2.86 (m, 1H), 2.76-2.66 (m, 1H), 2.62-2.55 (m, 2H), 2.47 (s, 3H), 2.00-1.88 (m, 1H), 1.85-1.75 (m, 1H), 1.74-1.66 (m, 1H), 1.65-1.54 (m, 1H), 0.91-0.70 (m, 4H).

### Example 596: N-[5-[2-cyano-5-[[3-(cyanomethylamino)cyclobutyl]methoxy]-4-pyridyl]pyrazolo[1,5-a]pyridin-2-yl]cyclopropanecarboxamide

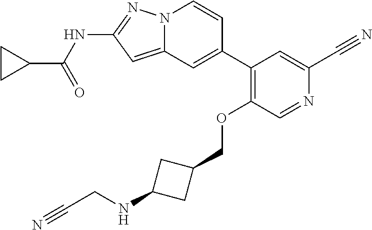 [SMILES: N#CCN[C@H]1C[C@@H](COc2cnc(C#N)cc2-c2ccn3nc(NC(=O)C4CC4)cc3c2)C1]

The title compound was prepared using methods analogous to those described in Example 5. MS (ESI): mass calcd. for C₂₄H₂₃N₇O₂, 441.2. m/z found, 442.2 [M+H]⁺. ¹H NMR (400 MHz, DMSO-d6) δ 11.14 (s, 1H), 8.65 (s, 1H), 8.62 (d, J=7.6 Hz, 1H), 8.20 (s, 1H), 7.93 (s, 1H), 7.11 (dd, J=2.0, 7.2 Hz, 1H), 6.93 (s, 1H), 4.27 (d, J=6.0 Hz, 2H), 3.51 (d, J=7.2 Hz, 2H), 3.25-3.13 (m, 1H), 2.70-2.62 (m, 1H), 2.45-2.35 (m, 1H), 2.34-2.25 (m, 2H), 1.98-1.89 (m, 1H), 1.68-1.57 (m, 2H), 0.87-0.79 (m, 4H).

### Example 597: N-[5-[2-cyano-5-[[3-(cyclopropylamino)cyclobutyl]methoxy]-4-pyridyl]pyrazolo[1,5-a]pyridin-2-yl]cyclopropanecarboxamide

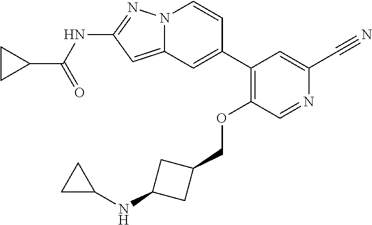 [SMILES: N#Cc1cc(-c2ccn3nc(NC(=O)C4CC4)cc3c2)c(OC[C@H]2C[C@@H](NC3CC3)C2)cn1]

The title compound was prepared using methods analogous to those described in Example 5. MS (ESI): mass calcd. for C₂₅H₂₆N₆O₂, 442.2. m/z found, 443.3 [M+H]⁺. ¹H NMR (400 MHz, DMSO-d6) δ 11.15 (s, 1H), 8.66-8.59 (m, 2H), 8.19 (s, 1H), 7.93 (s, 1H), 7.10 (dd, J=1.6, 7.2 Hz, 1H), 6.93 (s, 1H), 4.24 (d, J=6.4 Hz, 2H), 3.18-3.08 (m, 1H), 2.40-2.29 (m, 1H), 2.29-2.19 (m, 2H), 1.99-1.89 (m, 2H), 1.64-1.52 (m, 2H), 0.88-0.78 (m, 4H), 0.29-0.22 (m, 2H), 0.14-0.07 (m, 2H).

### Example 598: ethyl 2-[[3-[[6-cyano-4-[2-(cyclopropanecarbonylamino)pyrazolo[1,5-a]pyridin-5-yl]-3-pyridyl]oxymethyl]cyclobutyl]amino]acetate

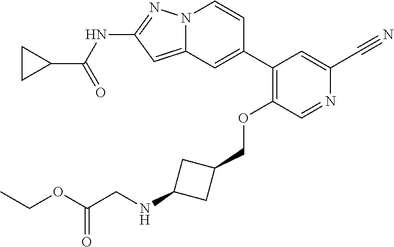 [SMILES: CCOC(=O)CN[C@H]1C[C@@H](COc2cnc(C#N)cc2-c2ccn3nc(NC(=O)C4CC4)cc3c2)C1]

The title compound was prepared using methods analogous to those described in Example 5. MS (ESI): mass calcd. for C₂₆H₂₈N₆O₄, 488.2. m/z found, 489.3 [M+H]⁺. ¹H NMR (400 MHz, DMSO-d6) δ 11.13 (s, 1H), 8.70-8.57 (m, 2H), 8.19 (s, 1H), 7.93 (s, 1H), 7.11 (dd, J=1.2 Hz, 7.2 Hz, 1H), 6.92 (s, 1H), 4.25 (d, J=6.0 Hz, 2H), 4.06 (q, J=7.2 Hz, 2H), 3.21 (s, 2H), 3.15-3.01 (m, 1H), 2.36-2.27 (m, 1H), 2.25-2.14 (m, 2H), 1.99-1.87 (m, 1H), 1.65-1.51 (m, 2H), 1.16 (t, J=6.8 Hz, 4H), 0.94-0.73 (m, 4H).

### Example 599: N-[5-[5-[[3-(benzylamino)cyclobutyl]methoxy]-2-cyano-4-pyridyl]pyrazolo[1,5-a]pyridin-2-yl]cyclopropanecarboxamide

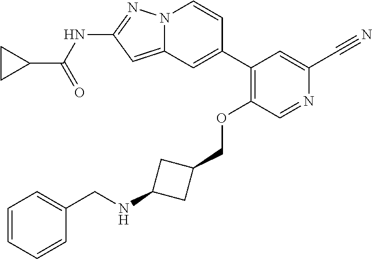 [SMILES: N#Cc1cc(-c2ccn3nc(NC(=O)C4CC4)cc3c2)c(OC[C@H]2C[C@@H](NCc3ccccc3)C2)cn1]

The title compound was prepared using methods analogous to those described in Example 5. MS (ESI): mass calcd. for C₂₉H₂₈N₆O₂, 492.2. m/z found, 493.3 [M+H]⁺. ¹H NMR (400 MHz, DMSO-d6) δ 11.14 (s, 1H), 8.67-8.55 (m, 2H), 8.18 (s, 1H), 7.92 (s, 1H), 7.31-7.23 (m, 4H), 7.22-7.16 (m, 1H), 7.10 (dd, J=1.6, 7.2 Hz, 1H), 6.94 (s, 1H), 4.24 (d, J=6.0 Hz, 2H), 3.57 (s, 2H), 3.15-2.99 (m, 1H), 2.39-2.27 (m, 1H), 2.26-2.15 (m, 2H), 2.14-2.02 (m, 1H), 2.00-1.84 (m, 1H), 1.62-1.48 (m, 2H), 0.93-0.76 (m, 4H).

### Example 600: 1-[4-[2-[(2,6-dimethylpyrimidin-4-yl)amino]pyrazolo[1,5-a]pyridin-5-yl]-6-methyl-pyridazin-3-yl]oxy-2-methyl-propan-2-ol

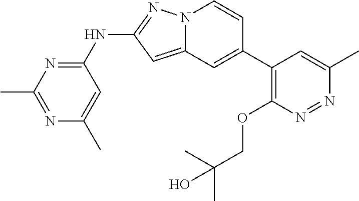

The title compound was prepared using methods analogous to those described in Example 488. MS (ESI): mass calcd. for C₂₂H₂₅N₇O₂, 419.2. m/z found, 420.2 [M+H]⁺. ¹H NMR (500 MHz, DMSO-d6) δ 10.23 (s, 1H), 8.65 (dd, J=7.2, 1.0 Hz, 1H), 8.04 (dd, J=2.0, 0.9 Hz, 1H), 7.73 (s, 1H), 7.16 (dd, J=7.2, 2.0 Hz, 1H), 7.02 (s, 1H), 6.88 (s, 1H), 4.69 (s, 1H), 4.28 (s, 2H), 2.59 (s, 3H), 2.46 (s, 3H), 2.30 (s, 3H), 1.20 (s, 6H).

### Example 601: N-[5-[5-[[(2S)-morpholin-2-yl]methoxy]-2-(trifluoromethyl)-4-pyridyl]pyrazolo[1,5-a]pyridin-2-yl]cyclopropanecarboxamide

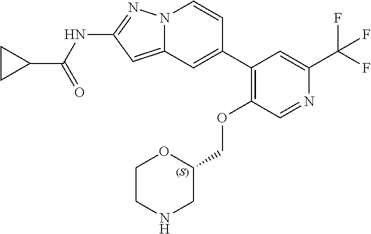

Step A: N-(5-(5-chloro-2-(trifluoromethyl)pyridin-4-yl)pyrazolo[1,5-a]pyridin-2-yl)cyclopropanecarboxamide. The title compound was prepared using methods analogous to those described in Intermediate 5 and using 5-chloro-4-iodo-2-(trifluoromethyl)pyridine instead of 4-bromo-5-chloropyridine-2-carbonitrile.

Step B: N-[5-[5-[[(2S)-morpholin-2-yl]methoxy]-2-(trifluoromethyl)-4-pyridyl]pyrazolo[1,5-a]pyridin-2-yl]cyclopropanecarboxamide. The title compound was prepared using methods analogous to those described in Example 136. MS (ESI): mass calcd. for C₂₂H₂₂F₃N₅O₃, 461.2. m/z found, 462.0 [M+H]⁺. ¹H NMR (400 MHz, DMSO-d6) δ 11.11 (s, 1H), 8.66 (s, 1H), 8.60 (dd, J=7.3, 1.0 Hz, 1H), 8.01 (d, J=2.0 Hz, 1H), 7.95 (s, 1H), 7.16 (dd, J=7.3, 2.1 Hz, 1H), 6.92 (s, 1H), 4.28 (d, J=4.8 Hz, 2H), 3.83-3.66 (m, 2H), 3.46 (td, J=10.8, 3.2 Hz, 1H), 2.82 (dd, J=12.2, 2.4 Hz, 1H), 2.72-2.58 (m, 2H), 2.58-2.51 (m, 1H), 2.44-2.31 (m, 1H), 2.01-1.84 (m, 1H), 0.91-0.73 (m, 4H). ¹⁹F NMR (376 MHz, DMSO) d −65.17.

### Example 602: (1*R, 3*S)—N-[5-[5-(3-hydroxycyclohexoxy)-2-methyl-4-pyridyl]pyrazolo[1,5-a]pyridin-2-yl]cyclopropanecarboxamide

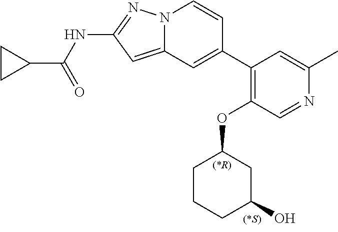 [SMILES: Cc1cc(-c2ccn3nc(NC(=O)C4CC4)cc3c2)c(O[C@@H]2CCC[C@H](O)C2)cn1]

The title compound was prepared using methods analogous to those described in Example 136. Purified by SFC over DAICEL CHIRALPAK AD (250 mm*30 mm, 10 um), (eluent: 45% to 45% (v/v) supercritical CO₂ in EtOH and H₂O with 0.1% NH₃). MS (ESI): mass calcd. for C₂₃H₂₆N₄O₃, 406.2. m/z found, 407.2 [M+H]⁺. ¹H NMR (400 MHz, DMSO-d6): 11.11 (s, 1H), 8.56 (d, J=7.6 Hz, 1H), 8.36 (s, 1H), 7.82 (s, 1H), 7.35 (s, 1H), 7.08-7.01 (m, 1H), 6.89 (s, 1H), 4.69 (d, J=4.4 Hz, 1H), 4.40-4.23 (m, 1H), 3.51-3.45 (m, 1H), 2.46 (s, 3H), 2.27-2.19 (m, 1H), 2.01-1.88 (m, 2H), 1.80-1.62 (m, 2H), 1.27-1.13 (m, 3H), 1.10-0.96 (m, 1H), 0.89-0.77 (m, 4H).

### Example 603: (*S)—N-[5-[5-(2-cyclopropyl-2-hydroxy-propoxy)-2-methyl-4-pyridyl]pyrazolo[1,5-a]pyridin-2-yl]cyclopropanecarboxamide

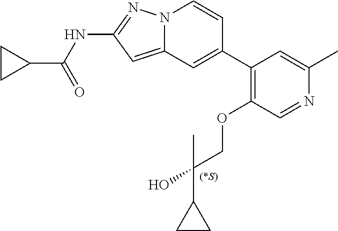 [SMILES: Cc1cc(-c2ccn3nc(NC(=O)C4CC4)cc3c2)c(OC[C@@](C)(O)C2CC2)cn1]

The title compound was prepared using methods analogous to those described in Example 136. Purified by chiral SFC with DAICEL CHIRALPAK AD (250 mm×30 mm, 10 um), column (eluent: 50% to 50% (v/v) EtOH with 0.1% NH₃—H₂O in supercritical CO₂). MS (ESI): mass calcd. for C₂₃H₂₆N₄O₃, 406.2. m/z found, 407.2 [M+H]⁺. ¹H NMR (400 MHz, DMSO-d6) δ 11.12 (s, 1H), 8.57 (d, J=7.2 Hz, 1H), 8.32 (s, 1H), 7.95-7.90 (m, 1H), 7.37 (s, 1H), 7.11 (dd, J=1.6, 6.8 Hz, 1H), 6.88 (s, 1H), 4.39 (s, 1H), 3.97-3.90 (m, 2H), 2.47 (s, 3H), 1.98-1.88 (m, 1H), 1.14 (s, 3H), 0.94-0.87 (m, 1H), 0.85-0.80 (m, 4H), 0.43-0.35 (m, 1H), 0.34-0.27 (m, 1H), 0.25-0.15 (m, 2H).

### Example 604: (*R)—N-[5-[5-[(1,2-dimethylazetidin-2-yl)methoxy]-2-methyl-4-pyridyl]pyrazolo[1,5-a]pyridin-2-yl]cyclopropanecarboxamide

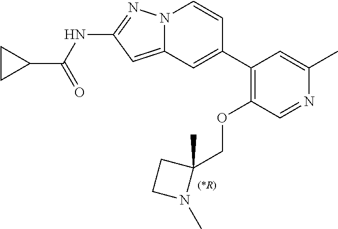 [SMILES: Cc1cc(-c2ccn3nc(NC(=O)C4CC4)cc3c2)c(OC[C@@]2(C)CCN2C)cn1]

The title compound was prepared using methods analogous to those described in Example 136. MS (ESI): mass calcd. for C₂₃H₂₇N₅O₂, 405.2. m/z found, 406.2 [M+H]⁺. ¹H NMR (400 MHz, CDCl₃) δ 8.32 (s, 1H), 8.28-8.23 (m, 2H), 7.70 (d, J=0.8 Hz, 1H), 7.16 (s, 1H), 6.98 (s, 1H), 6.95 (dd, J=2.0, 7.2 Hz, 1H), 4.00-3.91 (m, 2H), 3.37-3.30 (m, 1H), 3.04-2.96 (m, 1H), 2.55 (s, 3H), 2.19-2.10 (m, 4H), 1.80-1.75 (m, 1H), 1.60-1.52 (m, 1H), 1.24 (s, 3H), 1.18-1.12 (m, 2H), 0.95-0.88 (m, 2H).

### Example 605: N-[5-[5-[[(2S)-1-ethylazetidin-2-yl]methoxy]-2-methyl-4-pyridyl]pyrazolo[1,5-a]pyridin-2-yl]cyclopropanecarboxamide

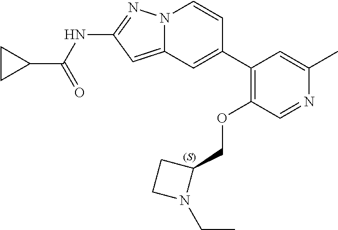

The title compound was prepared using methods analogous to those described in Example 451. MS (ESI): mass calcd. for C₂₃H₂₇N₅O₂, 405.2. m/z found, 406.3 [M+H]⁺. ¹H NMR (400 MHz, CDCl₃) δ 8.53 (s, 1H), 8.30-8.22 (m, 2H), 7.70 (s, 1H), 7.16 (s, 1H), 6.99 (s, 1H), 6.96 (dd, J=1.6, 7.2 Hz, 1H), 4.17-4.03 (m, 2H), 3.47-3.34 (m, 2H), 2.82-2.73 (m, 1H), 2.67-2.57 (m, 1H), 2.55 (s, 3H), 2.36-2.26 (m, 1H), 2.11-1.95 (m, 2H), 1.63-1.52 (m, 1H), 1.19-1.12 (m, 2H), 0.95-0.86 (m, 5H).

### Example 606: (3S,5S)-5-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol

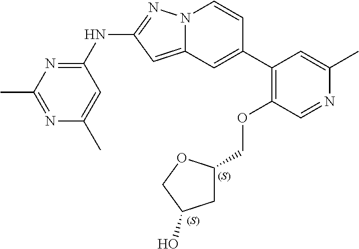

The title compound was prepared using a similar method described in Example 203. MS (ESI): mass calcd. for C₂₄H₂₆N₆O₃, 446.2. m/z found, 447.1 [M+H]⁺. ¹H NMR (400 MHz, DMSO-d6) δ 10.20 (s, 1H), 8.57 (d, J=7.2 Hz, 1H), 8.34 (s, 1H), 7.88 (d, J=1.2 Hz, 1H), 7.37 (s, 1H), 7.08 (dd, J=2.0, 7.2 Hz, 1H), 7.01 (s, 1H), 6.86 (s, 1H), 4.95 (d, J=3.6 Hz, 1H), 4.34-4.28 (m, 1H), 4.23-4.14 (m, 3H), 3.71-3.66 (m, 1H), 3.64-3.59 (m, 1H), 2.47 (s, 3H), 2.46 (s, 3H), 2.30 (s, 3H), 2.23-2.13 (m, 1H), 1.70-1.57 (m, 1H).

### Example 607: (*R)—N-(2,6-dimethylpyrimidin-4-yl)-5-[2-methyl-5-(7-oxaspiro[3.4]octan-6-ylmethoxy)-4-pyridyl]pyrazolo[1,5-a]pyridin-2-amine

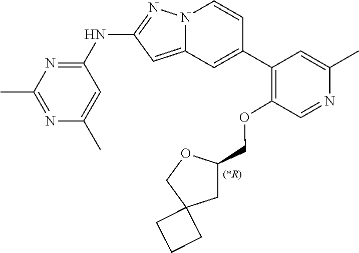

The title compound was prepared using a similar method described in Example 203. Purified by SFC-11 over DAICEL CHIRALPAK AD 250 mm×30 mm×10 μm (eluent: 45% to 45% (v/v) EtOH with 0.1% NH₃—H₂O in supercritical CO₂). MS (ESI): mass calcd. for C₂₇H₃₀N₆O₂, 470.2. m/z found, 471.2 [M+H]⁺. ¹H NMR (400 MHz, CDCl₃) δ 8.37 (d, J=6.8 Hz, 1H), 8.27 (s, 1H), 7.68 (d, J=0.8 Hz, 1H), 7.50 (s, 1H), 7.18 (s, 1H), 7.06 (s, 1H), 6.99 (dd, J=2.0, 7.2 Hz, 1H), 6.57 (s, 1H), 4.31-4.22 (m, 1H), 4.13-4.04 (m, 2H), 3.81-3.76 (m, 1H), 3.74-3.69 (m, 1H), 2.60 (s, 3H), 2.56 (s, 3H), 2.45 (s, 3H), 2.11-2.04 (m, 1H), 2.04-1.93 (m, 4H), 1.93-1.78 (m, 3H).

### Example 608: (*S)-3-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol

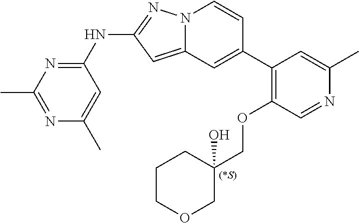

The title compound (53 mg, 37%) was prepared as in Example 216 and is the other isomer from the SFC purification. MS (ESI): mass calcd. for C₂₅H₂₈N₆O₃, 460.2. m/z found, 461.1 [M+H]⁺. ¹H NMR (400 MHz, CDCl₃) δ 8.30 (d, J=7.2 Hz, 1H), 8.28 (s, 1H), 7.58 (d, J=0.8 Hz, 1H), 7.56 (s, 1H), 7.16 (s, 1H), 7.02 (s, 1H), 6.85 (dd, J=2.0, 7.2 Hz, 1H), 6.57 (s, 1H), 4.11-4.06 (m, 1H), 4.06-4.01 (m, 1H), 3.81-3.73 (m, 1H), 3.68-3.61 (m, 2H), 3.61-3.53 (m, 1H), 3.03 (br. s., 1H), 2.56 (s, 3H), 2.56 (s, 3H), 2.44 (s, 3H), 1.96-1.86 (m, 1H), 1.81-1.70 (m, 2H), 1.61-1.54 (m, 1H).

### Example 609: (3S,5R)-5-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol

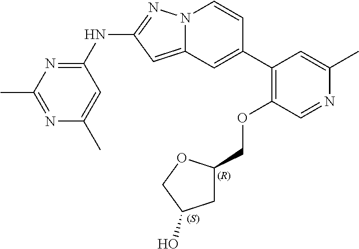

The title compound was prepared using a similar method described in Example 203. MS (ESI): mass calcd. for C₂₄H₂₆N₆O₃, 446.2. m/z found, 447.1 [M+H]⁺. ¹H NMR (400 MHz, CDCl₃) δ 8.30-8.26 (m, 2H), 7.63 (s, 1H), 7.60 (d, J=1.2 Hz, 1H), 7.17-7.13 (m, 2H), 6.95 (dd, J=2.0, 7.2 Hz, 1H), 6.48 (s, 1H), 4.63-4.55 (m, 1H), 4.53-4.48 (m, 1H), 4.22-4.17 (m, 1H), 4.16-4.10 (m, 1H), 3.96-3.91 (m, 1H), 3.84-3.79 (m, 1H), 2.60 (s, 3H), 2.55 (s, 3H), 2.46 (s, 3H), 2.38 (br. s., 1H), 2.10-2.02 (m, 1H), 2.01-1.91 (m, 1H).

### Example 610: (*S)—N-(2,6-dimethylpyrimidin-4-yl)-5-[5-(1,1-dioxothiazinan-4-yl)oxy-2-methyl-4-pyridyl]pyrazolo[1,5-a]pyridin-2-amine

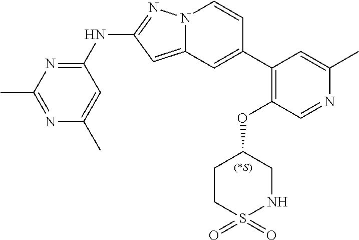

The title compound was prepared using a similar method described in Example 203. Purified by SFC with a DAICEL CHIRALPAK AS (250 mm×30 mm, 10 um) column (eluent: 35% to 25% (v/v) EtOH with 0.1% NH₃—H₂O in supercritical CO₂). MS (ESI): mass calcd. for C₂₃H₂₅N₇O₃S, 479.2. m/z found, 480.2 [M+H]⁺. ¹H NMR (400 MHz, DMSO-d6) δ 10.23 (s, 1H), 8.59 (d, J=7.2 Hz, 1H), 8.42 (s, 1H), 7.88 (s, 1H), 7.39 (s, 1H), 7.20 (t, J=6.4 Hz, 1H), 7.09 (dd, J=2.0, 7.2 Hz, 1H), 7.03 (br. s., 1H), 6.89 (br. s., 1H), 4.54-4.39 (m, 1H), 3.57-3.41 (m, 2H), 3.19-2.98 (m, 2H), 2.48 (s, 3H), 2.46 (s, 3H), 2.37-2.24 (m, 4H), 2.23-2.12 (m, 1H).

### Example 611: 5-[2-methyl-5-[[(2R)-4-methylmorpholin-2-yl]methoxy]-4-pyridyl]-N-pyrazin-2-yl-pyrazolo[1,5-a]pyridin-2-amine

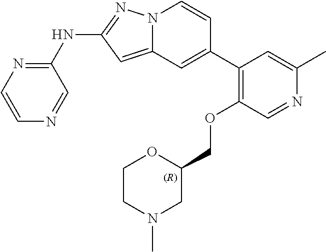

The title compound was prepared using a similar method described in Example 251, followed by a reductive amination step similar to that described in Example 449. MS (ESI): mass calcd. for C₂₃H₂₅N₇O₂, 431.2. m/z found, 432.2 [M+H]⁺. ¹H NMR (500 MHz, DMSO-d6) δ 10.26 (s, 1H), 8.65 (d, J=1.5 Hz, 1H), 8.60 (d, J=7.2 Hz, 1H), 8.34 (s, 1H), 8.21 (dd, J=2.8, 1.5 Hz, 1H), 8.01 (d, J=2.7 Hz, 1H), 7.87 (dd, J=2.1, 0.9 Hz, 1H), 7.36 (s, 1H), 7.04 (dd, J=7.2, 2.0 Hz, 1H), 6.91 (d, J=0.9 Hz, 1H), 4.18-4.10 (m, 2H), 3.83-3.71 (m, 2H), 3.56-3.46 (m, 1H), 2.76-2.69 (m, 1H), 2.61-2.55 (m, 1H), 2.47 (s, 3H), 2.15 (s, 3H), 2.01-1.92 (m, 1H), 1.92-1.85 (m, 1H).

### Example 612: 2-methyl-1-[[6-methyl-4-[2-[(6-methylpyridazin-3-yl)amino]pyrazolo[1,5-a]pyridin-5-yl]-3-pyridyl]oxy]propan-2-ol

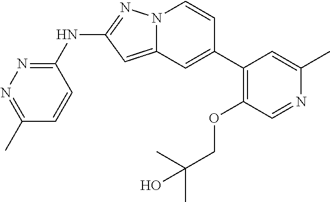 [SMILES: Cc1cc(-c2ccn3nc(Nc4ccc(C)nn4)cc3c2)c(OCC(C)(C)O)cn1]

The title compound was prepared using a similar method described in Example 200. MS (ESI): mass calcd. for C₂₂H₂₄N₆O₂, 404.2. m/z found, 405.20 [M+H]⁺. ¹H NMR (400 MHz, Chloroform-d) δ 9.81 (s, 1H), 8.28-8.21 (m, 1H), 8.12 (s, 1H), 7.80 (d, J=9.1 Hz, 1H), 7.49 (dd, J=2.0, 0.9 Hz, 1H), 7.10 (d, J=9.1 Hz, 1H), 7.03 (s, 1H), 6.75 (dd, J=7.2, 1.9 Hz, 1H), 6.62 (s, 1H), 3.83 (s, 2H), 3.39 (s, OH), 3.26 (s, 1H), 2.50 (s, 3H), 2.42 (s, 3H), 1.23 (s, 6H).

### Example 613: 1-[[4-[2-[(5-tert-butylpyrazin-2-yl)amino]pyrazolo[1,5-a]pyridin-5-yl]-6-methyl-3-pyridyl]oxy]-2-methyl-propan-2-ol

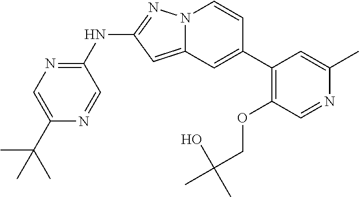 [SMILES: Cc1cc(-c2ccn3nc(Nc4cnc(C(C)(C)C)cn4)cc3c2)c(OCC(C)(C)O)cn1]

The title compound was prepared using a similar method described in Example 200. MS (ESI): mass calcd. for C₂₅H₃₀N₆O₂, 446.2. m/z found, 447.20 [M+H]⁺. ¹H NMR (400 MHz, Chloroform-d) δ 8.64 (d, J=1.5 Hz, 1H), 8.33 (dt, J=7.2, 0.9 Hz, 1H), 8.30-8.24 (m, 2H), 8.11 (s, 1H), 7.58 (dd, J=2.0, 0.9 Hz, 1H), 7.17 (s, 1H), 6.88 (dd, J=7.2, 1.9 Hz, 1H), 6.69 (d, J=0.8 Hz, 1H), 3.93 (s, 2H), 3.49 (s, 1H), 2.56 (s, 3H), 1.39 (s, 9H), 1.31 (s, 6H).

### Example 614: N-cyclopropyl-2,6-difluoro-4-[[5-[5-(4-hydroxycyclohexoxy)-2-methyl-4-pyridyl]pyrazolo[1,5-a]pyridin-2-yl]amino]benzamide

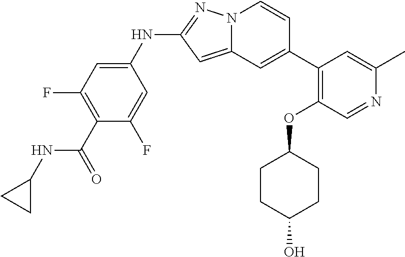 [SMILES: Cc1cc(-c2ccn3nc(Nc4cc(F)c(C(=O)NC5CC5)c(F)c4)cc3c2)c(O[C@H]2CC[C@H](O)CC2)cn1]

The title compound was prepared using a similar method described in Example 246. MS (ESI): mass calcd. for C₂₉H₂₉F₂N₅O₃, 533.2. m/z found, 534.3 [M+H]⁺. ¹H NMR (500 MHz, DMSO-d6) δ 9.64 (s, 1H), 8.64 (dt, J=7.2, 0.9 Hz, 1H), 8.50 (d, J=4.5 Hz, 1H), 8.35 (s, 1H), 7.72 (dd, J=2.0, 0.9 Hz, 1H), 7.33 (s, 1H), 7.24 (d, J=10.7 Hz, 2H), 6.95 (dd, J=7.2, 2.0 Hz, 1H), 6.19 (d, J=0.8 Hz, 1H), 4.51 (d, J=3.9 Hz, 1H), 4.41 (dt, J=8.9, 4.8 Hz, 1H), 3.49 (s, 1H), 2.81 (td, J=7.4, 3.9 Hz, 1H), 2.45 (s, 3H), 1.95 (d, J=5.4 Hz, 2H), 1.73 (d, J=9.3 Hz, 2H), 1.46-1.35 (m, 2H), 1.33-1.22 (m, 2H), 0.69 (td, J=7.1, 4.9 Hz, 2H), 0.52-0.45 (m, 2H).

### Example 615: N-cyclopropyl-2-fluoro-4-[[5-[5-(2-hydroxy-2-methyl-propoxy)-2-methyl-4-pyridyl]pyrazolo[1,5-a]pyridin-2-yl]amino]benzamide

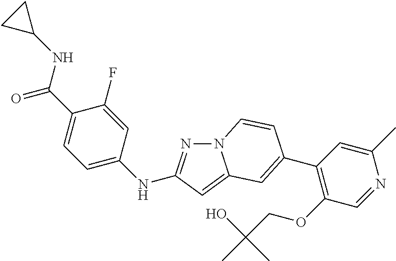 [SMILES: Cc1cc(-c2ccn3nc(Nc4ccc(C(=O)NC5CC5)c(F)c4)cc3c2)c(OCC(C)(C)O)cn1]

The title compound was prepared using a similar method described in Example 200. MS (ESI): mass calcd. for C₂₇H₂₈FN₅O₃, 489.2. m/z found, 490.2 [M+H]⁺. ¹H NMR (400 MHz, DMSO-d6) δ 9.56 (s, 1H), 8.63 (d, J=7.2 Hz, 1H), 8.31 (s, 1H), 7.93 (t, J=3.8 Hz, 1H), 7.80 (d, J=1.9 Hz, 1H), 7.63-7.50 (m, 2H), 7.36 (s, 1H), 7.19 (dd, J=8.6, 2.1 Hz, 1H), 7.01 (dd, J=7.2, 2.0 Hz, 1H), 6.16 (s, 1H), 4.62 (s, 1H), 3.87 (s, 2H), 2.81 (tq, J=7.7, 4.0 Hz, 1H), 2.46 (s, 3H), 1.15 (s, 6H), 0.68 (td, J=7.1, 4.6 Hz, 2H), 0.54 (dt, J=7.1, 4.5 Hz, 2H).

### Example 616: N-[5-[2-cyano-5-[[(3R)-1-ethylpyrrolidin-3-yl]methoxy]-4-pyridyl]pyrazolo[1,5-a]pyridin-2-yl]cyclopropanecarboxamide

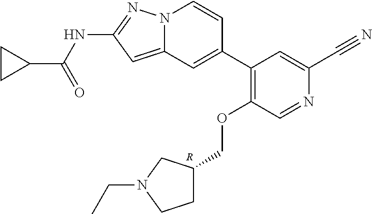

The title compound was prepared using methods analogous to those described in Example 5. MS (ESI): mass calcd. for C₂₄H₂₆N₆O₂, 430.2. m/z found, 431.2 [M+H]⁺. ¹H NMR (400 MHz, DMSO-d6) δ 11.14 (s, 1H), 8.67 (s, 1H), 8.62 (d, J=7.2 Hz, 1H), 8.19 (s, 1H), 7.94 (s, 1H), 7.10 (dd, J=1.6, 7.2 Hz, 1H), 6.91 (s, 1H), 4.21 (d, J=6.4 Hz, 2H), 2.59-2.56 (m, 1H), 2.46-2.41 (m, 2H), 2.39-2.30 (m, 3H), 2.03-1.86 (m, 3H), 1.59-1.40 (m, 1H), 0.98 (t, J=7.2 Hz, 3H), 0.87-0.79 (m, 4H).

### Example 617: N-(6-(5-(((1R,5S,7s)-3-oxa-9-azabicyclo[3.3.1]nonan-7-yl)oxy)-2-methylpyridin-4-yl)imidazo[1,2-a]pyridin-2-yl)cyclopropanecarboxamide

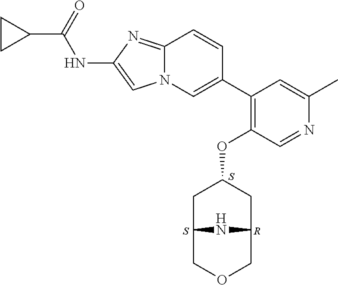

Step A: N-(6-bromoimidazo[1,2-a]pyridin-2-yl)cyclopropanecarboxamide: To a 0° C. solution of 6-bromoimidazo[1,2-a]pyridin-2-amine (1000 mg, 4.71 mmol) in DCM (20 mL), TEA (0.852 mL, 6.13 mmol) and cyclopropanecarbonyl chloride (591 mg, 5.66 mmol) were added. The solution was stirred for 2 h. The mixture was concentrated. The mixture was triturated with ether (100 mL), and solids were filtered and collected and dried providing the title compound (1.2 g, 91%). MS (ESI): mass calcd. for C₁₁H₁₀BrN₃O, 279.00. m/z found, 280.0 [M+H]⁺. ¹H NMR (500 MHz, Methanol-d4) δ 8.63 (t, J=1.4 Hz, 1H), 8.03 (s, 1H), 7.41-7.28 (m, 2H), 1.96-1.82 (m, 1H), 1.04-0.94 (m, 2H), 0.93-0.81 (m, 2H).

Step B: N-(6-(5-fluoro-2-methylpyridin-4-yl)imidazo[1,2-a]pyridin-2-yl)cyclopropanecarboxamide. The solid mixture N-(6-bromoimidazo[1,2-a]pyridin-2-yl)cyclopropanecarboxamide (1.10 g, 3.93 mmol), 5-fluoro-2-methyl-4-(4,4,5,5-tetramethyl-1,3,2-dioxaborolan-2-yl)pyridine[(1.21 g, 5.10 mmol), and chloro(2-dicyclohexylphosphino-2′,6′-diisopropoxy-1,1′-biphenyl)[2-(2′-amino-1,1′-biphenyl)]palladium(ii) (150 mg, 0.20 mmol) was evacuated and back-filled with N₂ three times. Then 1,4-dioxane (1.4 mL) and tribasic potassium phosphate 0.5 M in water (11.8 mL, 0.5 M, 5.9 mmol) were added. The mixture was stirred at 90° C. for 2 h. The crude was diluted with EtOAc (20 mL) and washed with H₂O (20 mL). The organic layers were combined, concentrated and purified with FFC (EtOAc:hexane) providing the title compound (510 mg, 41%). MS (ESI): mass calcd. for C₁₇H₁₅FN₄O, 310.12. m/z found, 311.0 [M+H]⁺. ¹H NMR (500 MHz, Methanol-d4) δ 8.80 (s, 1H), 8.40 (d, J=2.8 Hz, 1H), 8.13 (s, 1H), 7.62-7.49 (m, 3H), 2.9 (s, 3H), 1.98-1.85 (m, 1H), 1.06-0.96 (m, 2H), 0.96-0.87 (m, 2H).

Step C: N-(6-(5-(((1R,5S,7s)-3-oxa-9-azabicyclo[3.3.1]nonan-7-yl)oxy)-2-methylpyridin-4-yl)imidazo[1,2-a]pyridin-2-yl)cyclopropanecarboxamide. The mixture of endo (1R,5S,7s)-3-oxa-9-azabicyclo[3.3.1]nonan-7-ol hydrochloride (123 mg, 0.638 mmol) and NaH (60% dispersion in mineral oil) (100 mg, 2.50 mmol) in HMPA (1 mL) was stirred at 80° C. for 10 min, N-(6-(5-fluoro-2-methylpyridin-4-yl)imidazo[1,2-a]pyridin-2-yl)cyclopropanecarboxamide (97.0 mg, 0.312 mmol) was added. The mixture was stirred at 80° C. for 30 min. It was cooled down and water (2 mL) was added carefully. The mixture was extraction with DCM (2×20 mL). The combined organics were concentrated and purified with (MeOH/DCM) providing the title compound (47 mg, 34.7%). MS (ESI): mass calcd. for C₂₄H₂₇N₅O₃, 433.2. m/z found, 434.1 [M+H]⁺. ¹H NMR (500 MHz, Methanol-d4) δ 9.22 (dd, J=1.8, 0.9 Hz, 1H), 8.02 (s, 1H), 7.89 (s, 1H), 7.50 (dd, J=9.4, 1.8 Hz, 1H), 7.38-7.27 (m, 2H), 4.70-4.61 (m, 1H), 3.82-3.76 (m, 2H), 3.69-3.60 (m, 2H), 2.86 (d, J=6.8 Hz, 2H), 2.41 (s, 3H), 2.33-2.25 (m, 3H), 1.96-1.87 (m, 1H), 1.87-1.78 (m, 2H), 0.94-0.85 (m, 2H), 0.85-0.75 (m, 2H).

### Example 618: N-(5-(5-(((1R,5S,7s)-3-oxa-9-azabicyclo[3.3.1]nonan-7-yl)oxy)-2-(trifluoromethyl)pyridin-4-yl)pyrazolo[1,5-a]pyridin-2-yl)cyclopropanecarboxamide

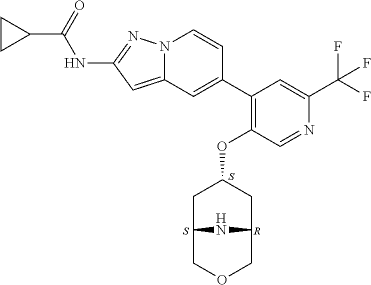

The title compound was prepared (150 mg, 34%) in the similar manner of Example 9. MS (ESI): mass calcd. for C₂₄H₂₄F₃N₅O₃, 487.2. m/z found, 488.0 [M+H]⁺. ¹H NMR (600 MHz, Methanol-d4) δ 8.49-8.26 (m, 3H), 8.11-8.00 (m, 1H), 7.80 (s, 1H), 7.13 (d, J=5.1 Hz, 1H), 4.95 (t, J=5.7 Hz, 1H), 3.95-3.77 (m, 4H), 3.60-3.49 (m, 2H), 2.70-2.53 (m, 2H), 2.28 (d, J=16.3 Hz, 2H), 1.85-1.75 (m, 1H), 0.94-0.75 (m, 4H).

### Example 619: N-(5-(5-(((1R,5S,7s)-9-methyl-3-oxa-9-azabicyclo[3.3.1]nonan-7-yl)oxy)-2-(trifluoromethyl)pyridin-4-yl)pyrazolo[1,5-a]pyridin-2-yl)cyclopropanecarboxamide

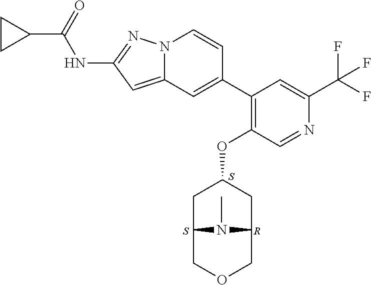

The title compound was prepared using methods analogous to those described in Example 449. MS (ESI): mass calcd. for C₂₅H₂₆F₃N₅O₃, 501.2. m/z found, 502.1 [M+H]⁺. ¹H NMR (500 MHz, Methanol-d4) δ 8.33-8.20 (m, 2H), 8.17 (d, J=2.0 Hz, 1H), 7.75 (s, 1H), 7.13 (dd, J=7.3, 2.0 Hz, 1H), 6.77 (s, 1H), 4.80 (t, J=6.4 Hz, 2H), 3.87-3.76 (m, 2H), 3.55 (d, J=11.2 Hz, 2H), 2.65-2.56 (m, 2H), 2.50-2.37 (m, 5H), 1.83-1.65 (m, 3H), 0.93-0.86 (m, 2H), 0.83-0.72 (m, 2H).

### Example 620: N-[5-[5-[(1-amino-3,3-difluoro-cyclobutyl)methoxy]-2-(trifluoromethyl)-4-pyridyl]pyrazolo[1,5-a]pyridin-2-yl]cyclopropanecarboxamide

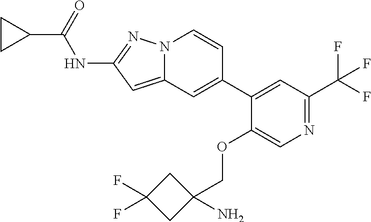 [SMILES: NC1(COc2cnc(C(F)(F)F)cc2-c2ccn3nc(NC(=O)C4CC4)cc3c2)CC(F)(F)C1]

The title compound was prepared using methods analogous to those described in Example 136. MS (ESI): mass calcd. for C₂₂H₂₀F₅N₅O₂, 481.2. m/z found, 482.0 [M+H]⁺. ¹H NMR (500 MHz, Methanol-d4) δ 8.58 (s, 1H), 8.50-8.40 (m, 1H), 7.87 (s, 1H), 7.82 (dd, J=2.1, 0.9 Hz, 1H), 7.06 (dd, J=7.2, 2.0 Hz, 1H), 6.94 (s, 1H), 4.25 (s, 2H), 2.89-2.71 (m, 2H), 2.59-2.39 (m, 2H), 1.95-1.75 (m, 1H), 1.06-0.95 (m, 2H), 0.95-0.80 (m, 2H).

### Example 621: N-[5-[5-[(3-fluoroazetidin-3-yl)methoxy]-2-(trifluoromethyl)-4-pyridyl]pyrazolo[1,5-a]pyridin-2-yl]cyclopropanecarboxamide

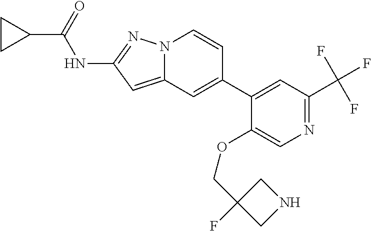 [SMILES: O=C(Nc1cc2cc(-c3cc(C(F)(F)F)ncc3OCC3(F)CNC3)ccn2n1)C1CC1]

The title compound was prepared using methods analogous to those described in Example 9. MS (ESI): mass calcd. for C₂₁H₁₉F₄N₅O₂, 449.1. m/z found, 450.0 [M+H]⁺. ¹H NMR (500 MHz, Methanol-d4) δ 8.62 (s, 1H), 8.48-8.36 (m, 1H), 7.90 (s, 1H), 7.83 (dd, J=2.1, 0.9 Hz, 1H), 7.06 (dd, J=7.3, 2.0 Hz, 1H), 6.93 (s, 1H), 4.64 (d, J=22.5 Hz, 2H), 3.86-3.72 (m, 2H), 3.72-3.54 (m, 2H), 2.01-1.81 (m, 1H), 1.00 (dt, J=4.6, 3.2 Hz, 2H), 0.95-0.80 (m, 2H).

### Example 622: N-[5-[5-[(3-methoxyazetidin-3-yl)methoxy]-2-(trifluoromethyl)-4-pyridyl]pyrazolo[1,5-a]pyridin-2-yl]cyclopropanecarboxamide

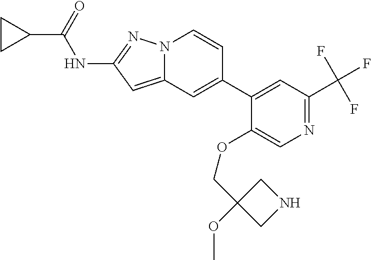 [SMILES: COC1(COc2cnc(C(F)(F)F)cc2-c2ccn3nc(NC(=O)C4CC4)cc3c2)CNC1]

The title compound was prepared using methods analogous to those described in Example 9. MS (ESI): mass calcd. for C₂₂H₂₂F₃N₅O₃, 461.2. m/z found, 462.1 [M+H]⁺. ¹H NMR (500 MHz, Methanol-d4) δ 8.62 (s, 1H), 8.48-8.36 (m, 1H), 7.90 (s, 1H), 7.83 (dd, J=2.1, 0.9 Hz, 1H), 7.06 (dd, J=7.3, 2.0 Hz, 1H), 6.93 (s, 1H), 4.64-4.50 (m, 2H), 3.78-3.65 (m, 2H), 3.52-3.34 (m, 2H), 3.27 (s, 3H), 2.01-1.81 (m, 1H), 1.05-0.95 (m, 2H), 0.95-0.80 (m, 2H).

### Example 623: 1-fluoro-N-[5-[2-methyl-5-[[(2R)-1-methylazetidin-2-yl]methoxy]-4-pyridyl]pyrazolo[1,5-a]pyridin-2-yl]cyclopropanecarboxamide

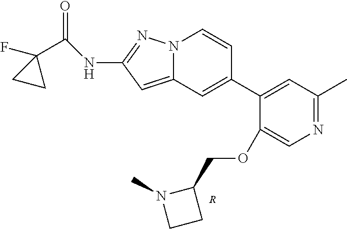

Step A; (R)-5-(2-methyl-5-((1-methylazetidin-2-yl)methoxy)pyridin-4-yl)pyrazolo[1,5-a]pyridin-2-amine. The crude title compound was prepared (450 mg, 28%) in the similar manner of Intermediate 13 while (R)-(1-methylazetidin-2-yl)methanol was used instead of tert-butyl (5)-2-(hydroxymethyl)morpholine-4-carboxylate. MS (ESI): mass calcd. for C₁₈H₂₁N₅O, 324.1. m/z found, 323.1 [M+H]⁺.

Step B: The mixture of (R)-5-(2-methyl-5-((1-methylazetidin-2-yl)methoxy)pyridin-4-yl)pyrazolo[1,5-a]pyridin-2-amine (126 mg, 0.39 mmol), 1-fluorocyclopropane-1-carboxylic acid (41 mg, 0.39 mmol), HATU (148 mg, 0.39 mmol) and Et₃N (0.16 mL, 1.17 mmol) in DCM (10 mL) was stirred at RT for 8 h. NaHCO₃ saturated solution (5 mL) was added. After separation, the organic layer was washed by NaOH solution (1M, 5 mL), concentrated and purified by column chromatography on silica (10% MeOH/DCM) providing the title compound (61 mg, 15%) MS (ESI): mass calcd. for C₂₂H₂₄FN₅O₂, 409.2. m/z found, 410.1 [M+H]⁺. ¹H NMR (500 MHz, Methanol-d4) δ 8.46 (dd, J=7.2, 1.0 Hz, 1H), 8.24 (s, 1H), 7.82 (dd, J=2.0, 0.9 Hz, 1H), 7.36 (s, 1H), 7.07 (dd, J=7.2, 2.0 Hz, 1H), 6.94 (d, J=0.8 Hz, 1H), 4.27-4.05 (m, 2H), 3.65-3.53 (m, 1H), 3.48-3.36 (m, 1H), 3.09-2.92 (m, 1H), 2.52 (s, 3H), 2.29 (s, 3H), 2.16-2.01 (m, 2H), 1.58-1.29 (m, 4H).

### Example 624: 2,2-difluoro-N-[5-[2-methyl-5-[[(2R)-1-methylazetidin-2-yl]methoxy]-4-pyridyl]pyrazolo[1,5-a]pyridin-2-yl]cyclopropanecarboxamide

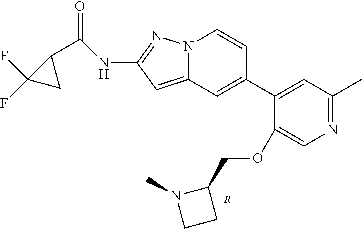

The title compound was prepared (55 mg, 41%) in the similar manner of Example 623 while 2,2-difluorocyclopropane-1-carboxylic acid was used instead of 1-fluorocyclopropane-1-carboxylic acid in Step B. MS (ESI): mass calcd. for C₂₂H₂₃F₂N₅O₂, 427.2. m/z found, 428.1 [M+H]⁺. ¹H NMR (500 MHz, Methanol-d4) δ 8.48-8.36 (m, 1H), 8.22 (s, 1H), 7.79 (dd, J=2.0, 0.9 Hz, 1H), 7.33 (s, 1H), 7.04 (dd, J=7.2, 2.0 Hz, 1H), 6.92 (d, J=0.9 Hz, 1H), 4.25-4.05 (m, 2H), 3.55-3.43 (m, 1H), 3.43-3.34 (m, 1H), 3.00-2.73 (m, 2H), 2.51 (s, 3H), 2.24 (s, 3H), 2.17-1.97 (m, 3H), 1.96-1.78 (m, 1H).

### Example 625: N-(2,6-dimethylpyrimidin-4-yl)-5-[5-[[(2R)-1-methylazetidin-2-yl]methoxy]-2-(trifluoromethyl)-4-pyridyl]pyrazolo[1,5-a]pyridin-2-amine

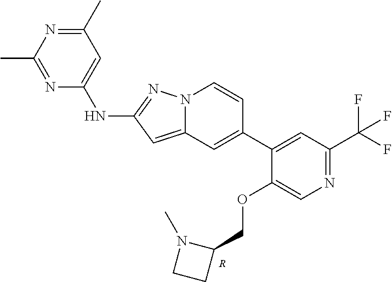

The title compound was prepared using methods analogous to those described in Example 251. MS (ESI): mass calcd. for C₂₄H₂₄F₃N₇O, 483.2. m/z found, 484.2 [M+H]⁺. ¹H NMR (500 MHz, Methanol-d4) δ 8.54 (s, 1H), 8.46 (dd, J=7.3, 1.0 Hz, 1H), 7.91-7.75 (m, 2H), 7.11-6.98 (m, 2H), 6.88 (s, 1H), 4.40-4.21 (m, 2H), 3.62-3.49 (m, 1H), 3.46-3.38 (m, 1H), 2.98-2.89 (m, 1H), 2.52 (s, 3H), 2.37 (s, 3H), 2.26 (s, 3H), 2.17-1.97 (m, 2H).

### Example 626: N-cyclopropyl-4-[[5-[5-(2-hydroxy-2-methyl-propoxy)-2-methyl-4-pyridyl]pyrazolo[1,5-a]pyridin-2-yl]amino]-2,6-dimethoxy-benzamide

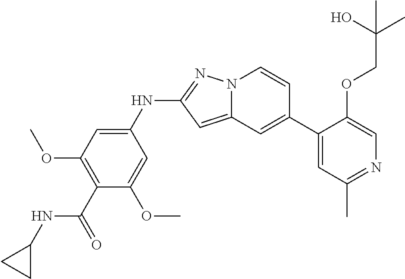 [SMILES: COc1cc(Nc2cc3cc(-c4cc(C)ncc4OCC(C)(C)O)ccn3n2)cc(OC)c1C(=O)NC1CC1]

The title compound was prepared (39 mg, 27%) by the similar method of Example 245 while Intermediate 20 was used instead of Intermediate 12. MS (ESI): mass calcd. for C₂₉H₃₃N₅O₅, 531.2. m/z found, [M+H]⁺. ¹H NMR (500 MHz, Methanol-d4) δ 8.44-8.35 (m, 1H), 8.22 (s, 1H), 7.71 (dd, J=2.0, 0.9 Hz, 1H), 7.37 (s, 1H), 6.99 (dd, J=7.2, 2.0 Hz, 1H), 6.83 (s, 2H), 6.17 (s, 1H), 3.92 (s, 2H), 3.82 (s, 6H), 2.87-2.72 (m, 1H), 2.53 (s, 3H), 1.26 (s, 6H), 0.84-0.73 (m, 2H), 0.62-0.54 (m, 2H).

### Example 627: 1-((1R,5S,7s)-7-((4-(2-((2,6-dimethylpyrimidin-4-yl)amino)pyrazolo[1,5-a]pyridin-5-yl)-6-methylpyridin-3-yl)oxy)-3-oxa-9-azabicyclo[3.3.1]nonan-9-yl)ethan-1-one

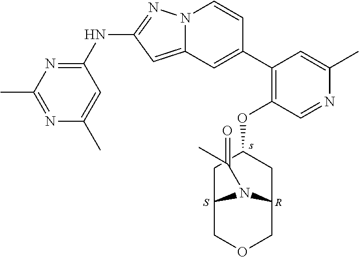

To the mixture of 5-(5-(((1R,5S,7s)-3-oxa-9-azabicyclo[3.3.1]nonan-7-yl)oxy)-2-methylpyridin-4-yl)-N-(2,6-dimethylpyrimidin-4-yl)pyrazolo[1,5-a]pyridin-2-amine (Example 382; 225 mg, 0.465 mmol) in DCM/DMF (10 mL/1 mL) at 0° C., acetic acid (33.4 mg, 0.538 mmol), HATU (230 mg, 0.604 mmol) and Et₃N (0.19 mL, 1.39 mmol) were added. The mixture was stirred at RT for 2 h. NaHCO₃ saturated solution (1 mL) was added. The mixture was extracted with DCM (2×2 mL). After separation, the organic layers were combined, concentrated and purified by column chromatography on silica (10% MeOH/DCM) providing the title compound (236 mg, 99%). MS (ESI): mass calcd. for C₂₈H₃₁N₇O₃, 513.2. m/z found, 514.2 [M+H]⁺. ¹H NMR (500 MHz, Methanol-d4) δ 8.26 (dd, J=7.3, 1.0 Hz, 1H), 8.01 (s, 1H), 7.90 (s, 1H), 7.24 (s, 1H), 7.02 (dd, J=7.2, 2.0 Hz, 1H), 6.90 (s, 1H), 6.68 (s, 1H), 4.50-4.34 (m, 2H), 3.94-3.88 (m, 1H), 3.71-3.64 (m, 2H), 3.56 (dd, J=11.3, 2.7 Hz, 1H), 3.47 (dd, J=11.3, 2.8 Hz, 1H), 2.40 (d, J=3.9 Hz, 6H), 2.32-2.22 (m, 4H), 2.22-2.11 (m, 1H), 2.01-1.83 (m, 5H).

### Example 628: 1-[(2S)-2-[[4-[2-[(2,6-dimethylpyrimidin-4-yl)amino]pyrazolo[1,5-a]pyridin-5-yl]-6-methyl-3-pyridyl]oxymethyl]morpholin-4-yl]ethanone

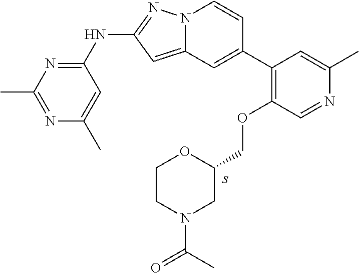

The title compound was prepared (61 mg, 100%) by the similar method of Example 627 while N-(2,6-dimethylpyrimidin-4-yl)-5-[2-methyl-5-[[(2S)-morpholin-2-yl]methoxy]-4-pyridyl]pyrazolo[1,5-a]pyridin-2-amine (Example 432) was used instead of 5-(5-(((1R,5S,7s)-3-oxa-9-azabicyclo[3.3.1]nonan-7-yl)oxy)-2-methylpyridin-4-yl)-N-(2,6-dimethylpyrimidin-4-yl)pyrazolo[1,5-a]pyridin-2-amine (Example 382). MS (ESI): mass calcd. for C₂₆H₂₉N₇O₃, 487.2. m/z found, 488.1 [M+H]⁺. ¹H NMR (500 MHz, Methanol-d4) δ 8.24 (t, J=7.5 Hz, 1H), 8.04 (d, J=9.9 Hz, 1H), 7.65-7.56 (m, 1H), 7.14 (d, J=4.1 Hz, 1H), 6.91-6.79 (m, 2H), 6.71-6.51 (m, 1H), 4.40-4.14 (m, 1H), 4.09-3.97 (m, 2H), 3.88-3.80 (m, 1H), 3.73-3.52 (m, 2H), 3.52-3.33 (m, 1H), 3.17-2.97 (m, 1H), 2.72-2.54 (m, 1H), 2.43-2.34 (m, 6H), 2.23 (s, 3H), 1.97-1.88 (s, 3H)

### Example 629: N-(2,6-dimethylpyrimidin-4-yl)-5-[2-methyl-5-[[(2S)-4-methylsulfonylmorpholin-2-yl]methoxy]-4-pyridyl]pyrazolo[1,5-a]pyridin-2-amine

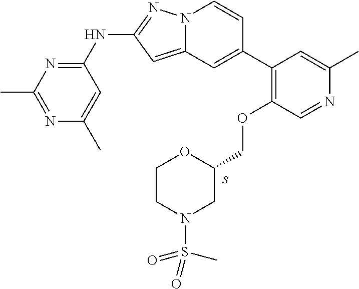

To the mixture of N-(2,6-dimethylpyrimidin-4-yl)-5-[2-methyl-5-[[(2S)-morpholin-2-yl]methoxy]-4-pyridyl]pyrazolo[1,5-a]pyridin-2-amine (Example 432, 50 mg, 0.112 mmol) in DCM/DMF (10 mL/1 mL) at 0° C., methylsulfonyl chloride (15 mg, 0.135 mmol), and Et₃N (0.047 mL, 0.34 mmol) were added. The mixture was stirred at RT for 2 h. NaHCO₃ saturated solution (1 mL) was added. The mixture was extracted with DCM (2×2 mL). After separation, the organic layers were combined, concentrated and purified by column chromatography on silica (10% MeOH/DCM) providing the title compound (17 mg, 27%). MS (ESI): mass calcd. for C₂₅H₂₉N₇O₄S, 523.2. m/z found, 524.2 [M+H]⁺. ¹H NMR (500 MHz, Methanol-d4) δ 8.37 (dd, J=7.2, 0.9 Hz, 1H), 8.16 (s, 1H), 7.72 (dd, J=2.0, 0.9 Hz, 1H), 7.29 (s, 1H), 6.98-6.90 (m, 2H), 6.81 (s, 1H), 4.15 (d, J=4.8 Hz, 2H), 4.05-3.96 (m, 1H), 3.87-3.79 (m, 1H), 3.68-3.58 (m, 2H), 3.50-3.40 (m, 1H), 2.89-2.77 (m, 1H), 2.77-2.73 (m, 4H), 2.53-2.47 (two S, 6H), 2.34 (s, 3H).

### Example 630: (S)-5-(2-methyl-5-((4-(methylsulfonyl)morpholin-2-yl)methoxy)pyridin-4-yl)-N-(2-methylpyrimidin-4-yl)pyrazolo[1,5-a]pyridin-2-amine

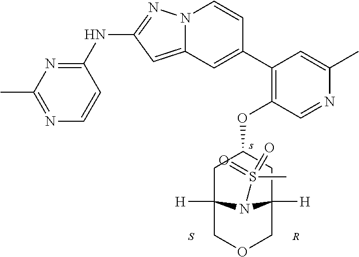

The title compound was prepared using methods analogous to those described in Example 361. MS (ESI): mass calcd. for C₂₆H₂₉N704S, 535.2. m/z found, 536.1 [M+H]⁺. ¹H NMR (500 MHz, Methanol-d4) δ 8.41 (d, J=7.1 Hz, 1H), 8.24-8.15 (m, 2H), 8.04 (d, J=2.0 Hz, 1H), 7.41 (d, J=1.4 Hz, 1H), 7.21-7.15 (m, 1H), 6.87 (s, 1H), 4.65 (dd, J=6.0, 4.3 Hz, 2H), 3.93 (d, J=7.4 Hz, 2H), 3.79-3.70 (m, 4H), 3.00 (s, 3H), 2.62-2.41 (m, 8H), 2.03-1.88 (m, 2H).

### Example 631: 2-((5-(5-(((1R,5S,7s)-9-acetyl-3-oxa-9-azabicyclo[3.3.1]nonan-7-yl)oxy)-2-methylpyridin-4-yl)pyrazolo[1,5-a]pyridin-2-yl)amino)-N-methylisonicotinamide

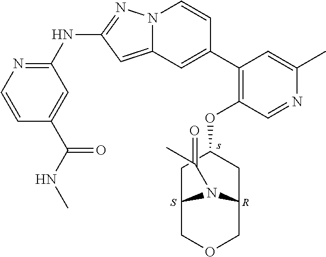

The title compound was prepared (82 mg, 94%) by the similar method of Example 627 while 2-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-N-methylisonicotinamide (Example 172) was used instead of 5-(5-(((1R,5S,7s)-3-oxa-9-azabicyclo[3.3.1]nonan-7-yl)oxy)-2-methylpyridin-4-yl)-N-(2,6-dimethylpyrimidin-4-yl)pyrazolo[1,5-a]pyridin-2-amine (Example 382). MS (ESI): mass calcd. for C₂₉H₃₁N₇O₄, 541.2. m/z found, 542.2 [M+H]⁺. ¹H NMR (500 MHz, Methanol-d4) δ 8.31-8.23 (m, 1H), 8.18 (dd, J=5.3, 0.8 Hz, 1H), 8.02 (s, 1H), 7.89 (dd, J=2.0, 0.9 Hz, 1H), 7.64-7.55 (m, 1H), 7.26 (s, 1H), 7.04-6.94 (m, 2H), 6.60 (d, J=0.9 Hz, 1H), 4.46-4.37 (m, 2H), 3.90 (d, J=7.0 Hz, 1H), 3.73-3.65 (m, 2H), 3.60-3.53 (m, 1H), 3.52-3.45 (m, 1H), 3.10 (q, J=7.3 Hz, 1H), 2.84 (s, 3H), 2.41 (s, 3H), 2.33-2.21 (m, 1H), 2.21-2.12 (m, 1H), 2.01-1.83 (m, 5H).

### Example 632: 1-((1R,5S,7s)-7-((6-methyl-4-(2-((4-(trifluoromethyl)pyridin-2-yl)amino)pyrazolo[1,5-a]pyridin-5-yl)pyridin-3-yl)oxy)-3-oxa-9-azabicyclo[3.3.1]nonan-9-yl)ethan-1-one

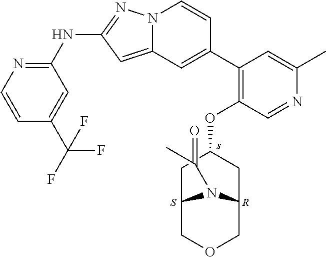

The title compound was prepared (15 mg, 61%) by the similar method of Example 627 while 5-(5-(((1R,5S,7s)-3-oxa-9-azabicyclo[3.3.1]nonan-7-yl)oxy)-2-methylpyridin-4-yl)-N-(4-(trifluoromethyl)pyridin-2-yl)pyrazolo[1,5-a]pyridin-2-amine (Example 378) was used instead of 5-(5-(((1R,5S,7s)-3-oxa-9-azabicyclo[3.3.1]nonan-7-yl)oxy)-2-methylpyridin-4-yl)-N-(2,6-dimethylpyrimidin-4-yl)pyrazolo[1,5-a]pyridin-2-amine (Example 382). MS (ESI): mass calcd. for C₂₈H₂₇F₃N₆O₃, 552.2. m/z found, 553.2 [M+H]⁺. ¹H NMR (500 MHz, Methanol-d4) δ 8.34-8.22 (m, 2H), 8.03 (s, 1H), 7.90 (dd, J=2.0, 0.9 Hz, 1H), 7.61-7.54 (m, 1H), 7.26 (s, 1H), 7.01 (dd, J=7.2, 2.0 Hz, 1H), 6.91 (dd, J=5.4, 1.5 Hz, 1H), 6.63 (d, J=0.8 Hz, 1H), 4.51-4.35 (m, 2H), 3.96-3.84 (m, 1H), 3.72-3.65 (m, 2H), 3.61-3.52 (m, 1H), 3.52-3.45 (m, 1H), 2.41 (s, 3H), 2.35-2.23 (m, 1H), 2.23-2.12 (m, 1H), 2.03-1.86 (m, 5H).

### Example 633: 2-[[5-[5-(2-hydroxy-2-methyl-propoxy)-2-methyl-4-pyridyl]pyrazolo[1,5-a]pyridin-2-yl]amino]-N,N,6-trimethyl-pyridine-4-carboxamide

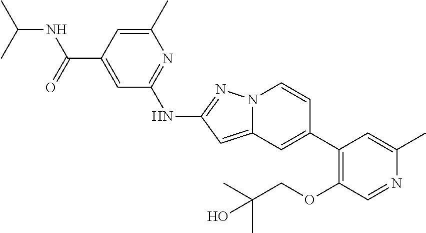 [SMILES: Cc1cc(-c2ccn3nc(Nc4cc(C(=O)NC(C)C)cc(C)n4)cc3c2)c(OCC(C)(C)O)cn1]

The title compound was prepared (60 mg, 39%) by the similar method of Example 244 while 2-chloro-6-methylpyridine-4-carboxylic acid, and isopropyl amine were used instead of 6-chloro-4-methylpicolinic acid and dimethyl amine. MS (ESI): mass calcd. for C₂₆H₃₀N₆O₃, 474.2. m/z found, 475.1 [M+H]⁺. ¹H NMR (500 MHz, Methanol-d4) δ 8.34-8.21 (m, 1H), 8.10 (s, 1H), 7.63 (dd, J=1.9, 0.9 Hz, 1H), 7.23 (s, 1H), 7.12 (t, J=1.0 Hz, 1H), 6.89 (dd, J=7.2, 2.0 Hz, 1H), 6.65 (d, J=0.8 Hz, 1H), 6.62-6.52 (m, 1H), 3.81 (s, 2H), 2.99 (s, 3H), 2.90 (s, 3H), 2.44-2.30 (two s, 6H), 2.04 (s, 2H), 1.15 (s, 6H).

### Example 634: 2-((5-(5-(((1R,5S,7s)-9-(cyclopropanecarbonyl)-3-oxa-9-azabicyclo[3.3.1]nonan-7-yl)oxy)-2-methylpyridin-4-yl)pyrazolo[1,5-a]pyridin-2-yl)amino)-N-methylisonicotinamide

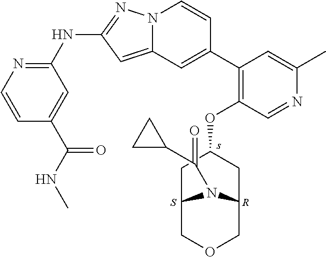

The title compound was prepared (40 mg, 88%) by the similar method of Example 627 while 2-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-N-methylisonicotinamide (Example 172) and cyclopropanecarboxylic acid were used instead of 5-(5-(((1R,5S,7s)-3-oxa-9-azabicyclo[3.3.1]nonan-7-yl)oxy)-2-methylpyridin-4-yl)-N-(2,6-dimethylpyrimidin-4-yl)pyrazolo[1,5-a]pyridin-2-amine and acetic acid. MS (ESI): mass calcd. for C₃₁H₃₃N₇O₄, 567.3. m/z found, 568.2 [M+H]⁺. ¹H NMR (500 MHz, Methanol-d4) δ 8.26 (dd, J=7.2, 1.0 Hz, 1H), 8.18 (dd, J=5.2, 0.8 Hz, 1H), 8.00 (s, 1H), 7.87 (dd, J=2.0, 0.9 Hz, 1H), 7.60 (t, J=1.1 Hz, 1H), 7.25 (s, 1H), 7.03-6.96 (m, 2H), 6.60 (d, J=0.8 Hz, 1H), 4.44-4.32 (m, 2H), 4.29 (d, J=7.3 Hz, 1H), 3.75-3.61 (m, 2H), 3.58 (dd, J=11.3, 2.7 Hz, 1H), 3.47 (dd, J=11.4, 2.8 Hz, 1H), 2.83 (s, 3H), 2.40 (s, 3H), 2.34-2.22 (m, 1H), 2.22-2.10 (m, 1H), 2.02-1.86 (m, 2H), 1.86-1.73 (m, 1H), 0.84-0.59 (m, 4H).

### Example 635: 2-((5-(5-(((1R,5S,7s)-9-(cyclopropylsulfonyl)-3-oxa-9-azabicyclo[3.3.1]nonan-7-yl)oxy)-2-methylpyridin-4-yl)pyrazolo[1,5-a]pyridin-2-yl)amino)-N-methylisonicotinamide

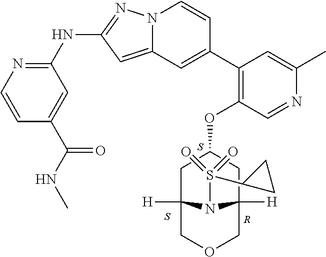

To a 0° C. solution of 2-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-N-methylisonicotinamide (Example 172, 40 mg, 0.08 mmol) in DCM/DMF (1 mL/0.2 mL) was added triethylamine (0.03 mL, 0.24 mmol). While stirring, added cyclopropanesulfonyl chloride (27 mg, 0.19 mmol) and stirred for 16 hr. Worked-up in DCM/sat′d NaHCO₃ and purified via FCC (10% MeOH/DCM) to give the title compound (40 mg, 82%). (MS (ESI): mass calcd. for C₃₀H₃₃N₇O₅S, 603.2. m/z found, 604.2 [M+H]⁺. ¹H NMR (400 MHz, Chloroform-d) δ 8.33-8.28 (m, 1H), 8.13 (s, 1H), 7.79-7.67 (m, 2H), 7.58 (s, 1H), 7.13 (s, 1H), 7.04-6.91 (m, 2H), 6.47 (d, J=0.8 Hz, 1H), 6.38 (d, J=4.8 Hz, 1H), 4.56-4.41 (m, 1H), 3.81 (d, J=7.2 Hz, 2H), 3.76-3.62 (m, 4H), 2.96 (d, J=4.3 Hz, 3H), 2.48 (s, 2H), 2.47-2.37 (m, 3H), 2.33-2.21 (m, 1H), 1.97-1.87 (m, 2H), 1.12-1.05 (m, 2H), 0.96-0.88 (m, 2H).

### Example 636: N-cyclopropyl-2-[[5-[5-(2-hydroxy-2-methyl-propoxy)-2-methyl-4-pyridyl]pyrazolo[1,5-a]pyridin-2-yl]amino]-6-methyl-pyridine-4-carboxamide

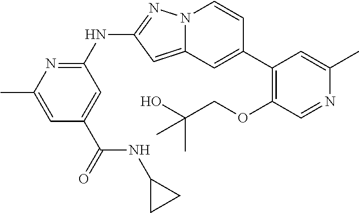

The title compound was prepared (12 mg, 15%) by the similar method of Example 244 while 2-chloro-6-methylpyridine-4-carboxylic acid and cyclopropylamine were used instead of 6-chloro-4-methylpicolinic acid and dimethylamine. MS (ESI): mass calcd. for C₂₇H₃₀N₆O3, 486.2. m/z found, 487.1 [M+H]⁺. ¹H NMR (400 MHz, Methanol-d4) δ 8.36-8.27 (m, 1H), 8.13 (s, 1H), 7.68 (dd, J=2.0, 0.9 Hz, 1H), 7.37 (dd, J=1.4, 0.7 Hz, 1H), 7.29 (s, 1H), 6.95 (dd, J=7.2, 1.9 Hz, 1H), 6.87 (dd, J=1.3, 0.6 Hz, 1H), 6.71 (d, J=0.8 Hz, 1H), 3.83 (s, 2H), 2.83-2.70 (m, 1H), 2.48-2.37 (2 s, 6H), 1.17 (s, 6H), 0.81-0.66 (m, 2H), 0.65-0.49 (m, 2H).

### Example 637: 6-((5-(5-(((1R,5S,7s)-9-acetyl-3-oxa-9-azabicyclo[3.3.1]nonan-7-yl)oxy)-2-methylpyridin-4-yl)pyrazolo[1,5-a]pyridin-2-yl)amino)-2-methylpyridazin-3(2H)-one

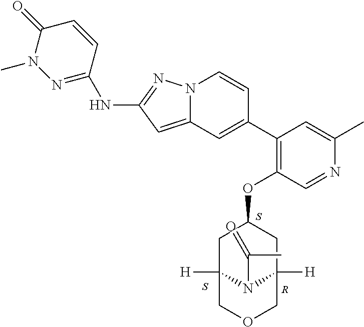

The title compound was prepared (39 mg, 94%) by the similar method of Example 627 while 6-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-2-methylpyridazin-3(2H)-one (Example 401) was used instead of 5-(5-(((1R,5S,7s)-3-oxa-9-azabicyclo[3.3.1]nonan-7-yl)oxy)-2-methylpyridin-4-yl)-N-(2,6-dimethylpyrimidin-4-yl)pyrazolo[1,5-a]pyridin-2-amine (Example 382). MS (ESI): mass calcd. for C₂₇H₂₉N₇O₄, 515.2. m/z found, 516.2 [M+H]⁺. ¹H NMR (400 MHz, Methanol-d4) δ 8.30-8.20 (m, 1H), 8.05 (s, 1H), 7.93 (dd, J=2.0, 0.9 Hz, 1H), 7.36-7.26 (m, 2H), 7.05 (dd, J=7.3, 2.0 Hz, 1H), 6.83 (d, J=9.7 Hz, 1H), 6.74 (d, J=0.9 Hz, 1H), 4.52-4.39 (m, 2H), 3.93 (d, J=7.2 Hz, 1H), 3.74-3.67 (m, 2H), 3.64 (s, 3H), 3.62-3.43 (m, 2H), 2.42 (s, 3H), 2.35-2.15 (m, 2H), 1.99 (s, 3H), 1.96-1.88 (m, 1H).

### Example 638: N-cyclopropyl-6-[[5-[5-(2-hydroxy-2-methyl-propoxy)-2-methyl-4-pyridyl]pyrazolo[1,5-a]pyridin-2-yl]amino]-4-methyl-pyridine-2-carboxamide

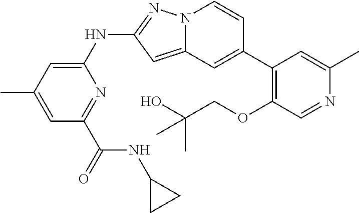

The title compound was prepared (45 mg, 29%) by the similar method of Example 244 while cyclopropylamine was used instead of dimethylamine. MS (ESI): mass calcd. for C₂₇H₃₀N₆O₃, 486.2. m/z found, 487.1 [M+H]⁺. ¹H NMR (500 MHz, Methanol-d4) δ 8.30 (dt, J=7.2, 0.9 Hz, 1H), 8.11 (s, 1H), 7.65 (dd, J=2.0, 0.9 Hz, 1H), 7.39-7.18 (m, 3H), 6.91 (dd, J=7.2, 2.0 Hz, 1H), 6.39 (d, J=0.8 Hz, 1H), 3.82 (s, 2H), 2.77 (tt, J=7.3, 3.9 Hz, 1H), 2.42 (s, 3H), 2.27 (t, J=0.7 Hz, 3H), 1.17 (s, 6H), 0.83-0.67 (m, 2H), 0.63-0.49 (m, 2H).

### Example 639: N-cyclopropyl-6-[[5-[5-(2-hydroxy-2-methyl-propoxy)-2-methyl-4-pyridyl]pyrazolo[1,5-a]pyridin-2-yl]amino]pyridine-3-carboxamide

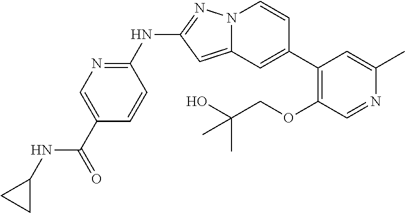 [SMILES: Cc1cc(-c2ccn3nc(Nc4ccc(C(=O)NC5CC5)cn4)cc3c2)c(OCC(C)(C)O)cn1]

The title compound was prepared (35 mg, 23%) by the similar method of Example 246 while 6-bromo-n-cyclopropylnicotinamide and Intermediate 20 were used instead of methyl 4-bromo-2-methoxybenzoate and Intermediate 12. MS (ESI): mass calcd. for C₂₆H₂₈N₆O₃, 472.2. m/z found, 487.1 [M+H]⁺. ¹H NMR (500 MHz, Methanol-d4) δ 8.69 (dd, J=2.4, 0.8 Hz, 1H), 8.51-8.39 (m, 1H), 8.24 (s, 1H), 8.04 (dd, J=8.8, 2.5 Hz, 1H), 7.81 (dd, J=2.0, 0.9 Hz, 1H), 7.44-7.32 (m, 2H), 7.07 (dd, J=7.2, 2.0 Hz, 1H), 6.83 (d, J=0.9 Hz, 1H), 3.95 (s, 2H), 2.91-2.83 (m, 1H), 2.55 (s, 3H), 1.28 (s, 6H), 0.88-0.77 (m, 2H), 0.70-0.59 (m, 2H).

### Example 640: N-cyclopropyl-4-[[5-[5-(2-hydroxy-2-methyl-propoxy)-2-methyl-4-pyridyl]pyrazolo[1,5-a]pyridin-2-yl]amino]-6-methyl-pyrimidine-2-carboxamide

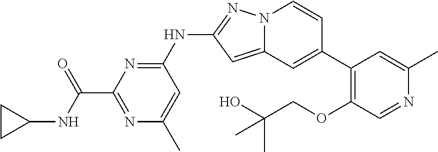 [SMILES: Cc1cc(-c2ccn3nc(Nc4cc(C)nc(C(=O)NC5CC5)n4)cc3c2)c(OCC(C)(C)O)cn1]

The title compound was prepared (52 mg, 55%) by the similar method of Example 241 while methyl 4-chloro-6-methylpyrimidine-2-carboxylate, Intermediate 20 and cyclopropylamine were used instead of methyl 4-bromo-2,6-dimethoxybenzoate, Intermediate 12 and 2,2,2-trifluoroethan-1-amine. MS (ESI): mass calcd. for C₂₆H₂₉N₇O₃, 487.2. m/z found, 488.3 [M+H]⁺. ¹H NMR (500 MHz, Methanol-d4) δ 8.31 (d, J=7.2 Hz, 1H), 8.09 (s, 1H), 7.65 (dd, J=2.0, 0.9 Hz, 1H), 7.40-7.28 (m, 1H), 7.21 (s, 1H), 6.94 (dd, J=7.2, 2.0 Hz, 1H), 6.49 (s, 1H), 3.82 (s, 2H), 2.85-2.70 (m, 1H), 2.37 (d, J=28.3 Hz, 6H), 1.16 (s, 6H), 0.80-0.71 (m, 2H), 0.63-0.54 (m, 2H).

### Example 641: 3-[[6-(difluoromethoxy)-4-[2-[(2-methylpyrimidin-5-yl)amino]pyrazolo[1,5-a]pyridin-5-yl]-3-pyridyl]oxy]-2,2-dimethyl-propanenitrile

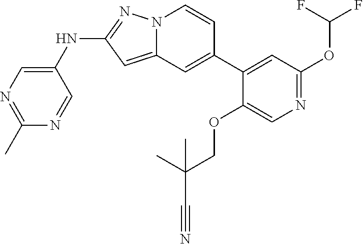 [SMILES: Cc1ncc(Nc2cc3cc(-c4cc(OC(F)F)ncc4OCC(C)(C)C#N)ccn3n2)cn1]

The title compound was prepared using a similar method described in Example 682, except tBuXPhos Pd G3 was used as the catalyst. MS (ESI): mass calcd. for C₂₃H₂₁F₂N₇O₂, 465.2. m/z found, 466.1 [M+H]⁺. ¹H NMR (500 MHz, DMSO-d₆) δ 9.29 (s, 1H), 8.92 (s, 2H), 8.62 (dd, J=7.2, 0.9 Hz, 1H), 8.14 (s, 1H), 7.83 (dd, J=2.0, 0.9 Hz, 1H), 7.66 (t, J=73.2 Hz, 1H), 7.26 (s, 1H), 6.98 (dd, J=7.2, 2.0 Hz, 1H), 6.15 (d, J=0.8 Hz, 1H), 4.20 (s, 2H), 2.54 (s, 3H), 1.35 (s, 6H).

### Example 642: methyl 4-[[5-[5-(2-hydroxy-2-methyl-propoxy)-2-methyl-4-pyridyl]pyrazolo[1,5-a]pyridin-2-yl]amino]-2-methoxy-benzoate

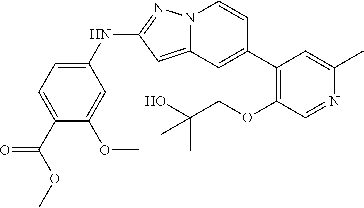 [SMILES: COC(=O)c1ccc(Nc2cc3cc(-c4cc(C)ncc4OCC(C)(C)O)ccn3n2)cc1OC]

The title compound was prepared (86 mg, 64%) by the similar method of Example 246 while Intermediate 20 was used instead of Intermediate 18. MS (ESI): mass calcd. for C₂₆H₂₈N₄O₅, 476.2. m/z found, 477.2 [M+H]⁺. ¹H NMR (500 MHz, Methanol-d4) δ 8.34-8.26 (m, 1H), 8.11 (s, 1H), 7.66 (d, J=8.7 Hz, 1H), 7.61 (dd, J=2.0, 0.9 Hz, 1H), 7.28 (d, J=2.1 Hz, 1H), 7.24 (s, 1H), 6.94-6.83 (m, 2H), 6.09 (d, J=0.8 Hz, 1H), 3.86-3.77 (m, 5H), 3.71 (s, 3H), 2.41 (s, 3H), 1.16 (s, 6H).

### Example 643: N-cyclopropyl-4-[[5-[5-(2-hydroxy-2-methyl-propoxy)-2-methyl-4-pyridyl]pyrazolo[1,5-a]pyridin-2-yl]amino]-2-methoxy-benzamide

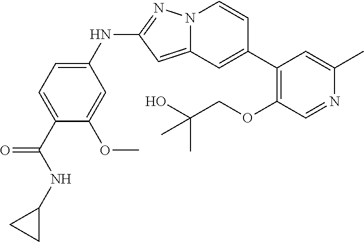 [SMILES: COc1cc(Nc2cc3cc(-c4cc(C)ncc4OCC(C)(C)O)ccn3n2)ccc1C(=O)NC1CC1]

The title compound was prepared (42 mg, 48%) by the similar method of Example 241 while Intermediate 20, and cyclopropylamine were used instead of Intermediate 12 and 2,2,2-trifluoroethan-1-amine. MS (ESI): mass calcd. for C₂₈H₃₁N₅O₄, 501.2. m/z found, [M+H]⁺. ¹H NMR (500 MHz, Methanol-d4) δ 8.39-8.23 (m, 1H), 8.12 (s, 1H), 7.79 (d, J=8.7 Hz, 1H), 7.63 (dd, J=2.0, 0.9 Hz, 1H), 7.35 (d, J=2.1 Hz, 1H), 7.27 (s, 1H), 6.97-6.85 (m, 2H), 6.10 (d, J=0.8 Hz, 1H), 3.90 (s, 3H), 3.82 (s, 2H), 2.79-2.67 (m, 1H), 2.43 (s, 3H), 1.16 (s, 6H), 0.77-0.66 (m, 2H), 0.54-0.45 (m, 2H).

### Example 644: 4-[[5-[5-(2-hydroxy-2-methyl-propoxy)-2-methyl-4-pyridyl]pyrazolo[1,5-a]pyridin-2-yl]amino]-6-methyl-N-(2,2,2-trifluoroethyl)pyrimidine-2-carboxamide

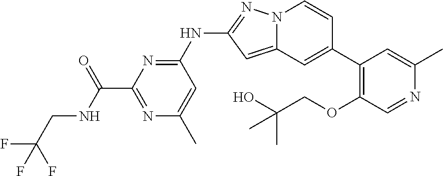 [SMILES: Cc1cc(-c2ccn3nc(Nc4cc(C)nc(C(=O)NCC(F)(F)F)n4)cc3c2)c(OCC(C)(C)O)cn1]

The title compound was prepared (43 mg, 47%) by the similar method of Example 241 while methyl 4-chloro-6-methylpyrimidine-2-carboxylate, and Intermediate 20 were used instead of methyl 4-bromo-2,6-dimethoxybenzoate and Intermediate 12. MS (ESI): mass calcd. for C₂₅H₂₆F₃N₇O₃, 529.2. m/z found, 530.2 [M+H]⁺. ¹H NMR (500 MHz, Methanol-d4) δ 8.46-8.31 (m, 1H), 8.13 (s, 1H), 7.71 (dd, J=1.9, 0.9 Hz, 1H), 7.44 (s, 1H), 7.27 (s, 1H), 6.99 (dd, J=7.2, 2.0 Hz, 1H), 6.63 (s, 1H), 4.05 (q, J=9.3 Hz, 2H), 3.83 (s, 2H), 2.48-2.37 (m, 6H), 1.17 (s, 6H).

### Example 645: (1r,4r)-4-((4-(2-((3,5-dimethoxyphenyl)amino)pyrazolo[1,5-a]pyridin-5-yl)-6-methylpyridin-3-yl)oxy)cyclohexan-1-ol

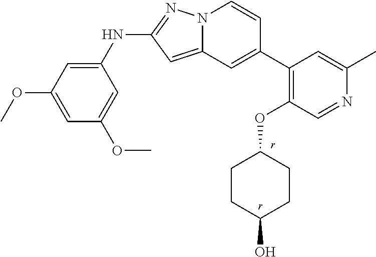

The title compound was prepared (55 mg, 24%) by the similar method of Example 246 while 1-bromo-3,5-dimethoxybenzene was used instead of methyl 4-bromo-2-methoxybenzoate. MS (ESI): mass calcd. for C₂₇H₃₀N₄O₄, 474.2. m/z found, 475.1 [M+H]⁺. ¹H NMR (500 MHz, Methanol-d4) δ 8.21-8.17 (m, 1H), 8.08 (s, 1H), 7.44 (dd, J=1.9, 0.9 Hz, 1H), 7.17 (s, 1H), 6.74 (dd, J=7.2, 1.9 Hz, 1H), 6.55 (d, J=2.2 Hz, 2H), 6.01 (d, J=0.8 Hz, 1H), 5.93 (t, J=2.2 Hz, 1H), 4.30-4.16 (m, 1H), 3.65 (s, 6H), 3.56-3.43 (m, 1H), 2.38 (s, 3H), 2.01-1.90 (m, 2H), 1.81-1.70 (m, 2H), 1.44-1.32 (m, 2H), 1.32-1.18 (m, 2H).

### Example 646: 1-[[4-[2-(3,5-dimethoxyanilino)pyrazolo[1,5-a]pyridin-5-yl]-6-methyl-3-pyridyl]oxy]-2-methyl-propan-2-ol

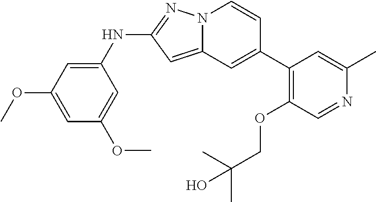

The title compound was prepared (35 mg, 24%) by the similar method of Example 246 while 1-bromo-3,5-dimethoxybenzene and Intermediate 20 were used instead of methyl 4-bromo-2-methoxybenzoate and Intermediate 12. MS (ESI): mass calcd. for C₂₅H₂₈N₄O₄, 448.2. m/z found, 449.1 [M+H]⁺. ¹H NMR (500 MHz, Methanol-d4) δ 8.26 (d, J=7.2 Hz, 1H), 8.11 (s, 1H), 7.58 (dd, J=1.9, 0.8 Hz, 1H), 7.26 (s, 1H), 6.86 (dd, J=7.2, 1.9 Hz, 1H), 6.56 (d, J=2.2 Hz, 2H), 6.05 (s, 1H), 5.94 (t, J=2.2 Hz, 1H), 3.81 (s, 2H), 3.74-3.65 (m, 6H), 2.42 (s, 3H), 1.16 (s, 6H).

### Example 647: 4-[[5-[5-(2-hydroxy-2-methyl-propoxy)-2-methyl-4-pyridyl]pyrazolo[1,5-a]pyridin-2-yl]amino]-N-(2-hydroxy-2-methyl-propyl)-6-methyl-pyrimidine-2-carboxamide

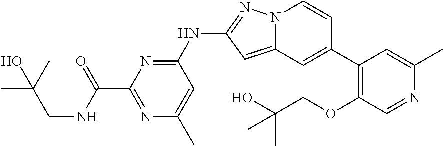 [SMILES: Cc1cc(-c2ccn3nc(Nc4cc(C)nc(C(=O)NCC(C)(C)O)n4)cc3c2)c(OCC(C)(C)O)cn1]

The title compound was prepared (17 mg, 17%) by the similar method of Example 241 while methyl 4-chloro-6-methylpyrimidine-2-carboxylate, Intermediate 20 and 1-amino-2-methylpropan-2-ol were used instead of methyl 4-bromo-2,6-dimethoxybenzoate, Intermediate 12 and 2,2,2-trifluoroethan-1-amine. MS (ESI): mass calcd. for C₂₇H₃₃N₇O₄, 519.3. m/z found, 519.3 [M+H]⁺. ¹H NMR (500 MHz, Methanol-d4) δ 8.40-8.30 (m, 1H), 8.13 (s, 1H), 7.74 (dd, J=2.0, 0.9 Hz, 1H), 7.30 (s, 1H), 7.04-6.93 (m, 1H), 6.92 (s, 1H), 6.76 (s, 1H), 4.47 (s, 1H), 3.83 (s, 2H), 2.43 (s, 3H), 2.36-2.31 (m, 4H), 1.17 (s, 6H), 0.81 (d, J=6.8 Hz, 3H), 0.75 (d, J=6.8 Hz, 3H).

### Example 648: N-cyclopropyl-2-(difluoromethoxy)-4-[[5-[5-(2-hydroxy-2-methyl-propoxy)-2-methyl-4-pyridyl]pyrazolo[1,5-a]pyridin-2-yl]amino]-6-methoxy-benzamide

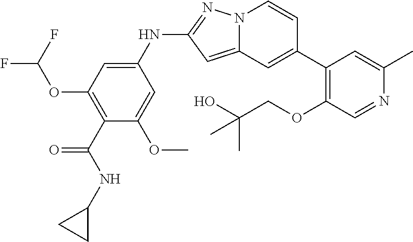 [SMILES: COc1cc(Nc2cc3cc(-c4cc(C)ncc4OCC(C)(C)O)ccn3n2)cc(OC(F)F)c1C(=O)NC1CC1]

The title compound was prepared (110 mg, 55%) by the similar method of Example 333 while Intermediate 20 was used instead of Intermediate 12. MS (ESI): mass calcd. for C₂₉H₃₁F₂N₅O₅, 567.2. m/z found, 568.2 [M+H]⁺. ¹H NMR (600 MHz, CD₃OD) δ 8.49-8.35 (m, 1H), 8.23 (s, 1H), 7.72 (dd, J=2.0, 0.9 Hz, 1H), 7.37 (s, 1H), 7.14 (d, J=1.9 Hz, 1H), 7.04-6.96 (m, 2H), 6.76 (t, J=74.4 Hz, 1H), 6.16 (s, 1H), 3.93 (s, 2H), 3.88 (s, 3H), 2.91-2.76 (m, 1H), 2.54 (s, 3H), 1.28 (s, 6H), 0.87-0.77 (m, 2H), 0.66-0.55 (m, 2H).

### Example 649: N-cyclopropyl-2-fluoro-4-((5-(5-4(1r,4r)-4-hydroxycyclohexyl)oxy)-2-methylpyridin-4-yl)pyrazolo[1,5-a]pyridin-2-yl)amino)-6-methoxybenzamide

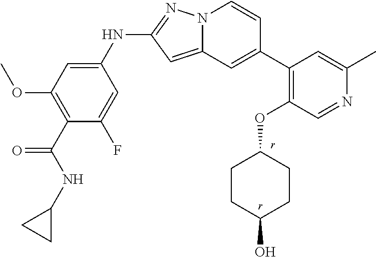

The title compound was prepared (81 mg, 46%) by the similar method of Example 245 while methyl 4-bromo-2-fluoro-6-methoxybenzoic acid was used instead of 4-bromo-2,6-dimethoxybenzoic acid. MS (ESI): mass calcd. for C₃₀H₃₂FN₅O₄, 545.2. m/z found, 546.2 [M+H]⁺. ¹H NMR (500 MHz, CD₃OD) δ 8.36-8.26 (m, 1H), 8.12 (s, 1H), 7.53 (dd, J=2.0, 0.9 Hz, 1H), 7.23 (s, 1H), 6.96-6.87 (m, 1H), 6.87-6.78 (m, 2H), 6.05 (d, J=0.8 Hz, 1H), 4.37-4.21 (m, 1H), 3.76 (s, 3H), 3.59-3.48 (m, 1H), 2.76-2.66 (m, 1H), 2.41 (s, 3H), 2.02-1.89 (m, 2H), 1.82-1.68 (m, 2H), 1.47-1.35 (m, 2H), 1.35-1.22 (m, 2H), 0.74-0.63 (m, 2H), 0.55-0.42 (m, 2H).

### Example 650: 6-[[5-[5-(2-hydroxy-2-methyl-propoxy)-2-methyl-4-pyridyl]pyrazolo[1,5-a]pyridin-2-yl]amino]-N-isopropyl-4-(trifluoromethyl)pyridine-3-carboxamide

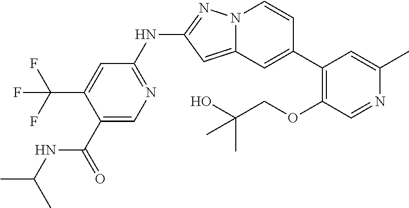 [SMILES: Cc1cc(-c2ccn3nc(Nc4cc(C(F)(F)F)c(C(=O)NC(C)C)cn4)cc3c2)c(OCC(C)(C)O)cn1]

The title compound was prepared (52 mg, 72%) by the similar method of Example 241 while methyl 6-chloro-4-(trifluoromethyl)nicotinate, Intermediate 20 and isopropyl amine were used instead of methyl 4-bromo-2,6-dimethoxybenzoate, Intermediate 12 and 2,2,2-trifluoroethan-1-amine. MS (ESI): mass calcd. for C₂₇H₂₉F₃N₆O₃, 542.2. m/z found, 543.2 [M+H]⁺. ¹H NMR (500 MHz, CDCl₃) δ 8.34 (s, 1H), 8.22 (d, J=7.2 Hz, 1H), 8.13 (s, 1H), 7.55 (s, 1H), 7.47 (s, 1H), 7.02 (s, 1H), 6.79 (dd, J=7.2, 1.9 Hz, 1H), 6.38 (s, 1H), 5.94 (d, J=8.0 Hz, 1H), 4.24-4.13 (m, 1H), 3.85 (s, 2H), 2.45 (s, 3H), 1.24 (s, 6H) 1.19 (d, J=6.6 Hz, 6H).

### Example 651: 6-[[5-[5-(2-hydroxy-2-methyl-propoxy)-2-methyl-4-pyridyl]pyrazolo[1,5-a]pyridin-2-yl]amino]-N-(2,2,2-trifluoroethyl)-4-(trifluoromethyl)pyridine-3-carboxamide

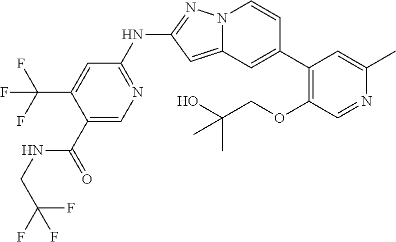 [SMILES: Cc1cc(-c2ccn3nc(Nc4cc(C(F)(F)F)c(C(=O)NCC(F)(F)F)cn4)cc3c2)c(OCC(C)(C)O)cn1]

The title compound was prepared (73 mg, 95%) by the similar method of Example 241 while methyl 6-chloro-4-(trifluoromethyl)nicotinate, and Intermediate 20 were used instead of methyl 4-bromo-2,6-dimethoxybenzoate, and Intermediate 12. MS (ESI): mass calcd. for C₂₆H₂₄F₆N₆O₃, 582.2. m/z found, 583.1 [M+H]⁺. ¹H NMR (500 MHz, CDCl₃) δ 8.44 (s, 1H), 8.30 (d, J=7.1 Hz, 1H), 8.19 (s, 1H), 7.81 (s, 1H), 7.54 (d, J=8.5 Hz, 2H), 7.10 (s, 1H), 6.88 (dd, J=7.2, 1.8 Hz, 1H), 6.44 (s, 1H), 6.10 (s, 1H), 4.15-3.99 (m, 2H), 3.85 (s, 3H), 2.49 (s, 3H), 1.23 (s, 6H).

### Example 652: 1-[[4-[2-[(3-fluoro-4-methyl-2-pyridyl)amino]pyrazolo[1,5-a]pyridin-5-yl]-6-methyl-3-pyridyl]oxy]-2-methyl-propan-2-ol

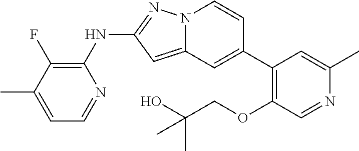

The title compound was prepared (55 mg, 34%) by the similar method of Example 246 while 2-bromo-3-fluoro-4-picoline and Intermediate 20 were used instead of methyl 4-bromo-2-methoxybenzoate and Intermediate 12. MS (ESI): mass calcd. for C₂₃H₂₄FN₅O₂, 421.2. m/z found, 422.1 [M+H]⁺. ¹H NMR (500 MHz, CD₃OD) δ 8.37-8.27 (m, 1H), 8.18 (s, 1H), 7.86 (d, J=5.1 Hz, 1H), 7.71 (dd, J=2.0, 0.9 Hz, 1H), 7.28 (s, 1H), 7.05-6.93 (m, 2H), 6.74-6.60 (m, 1H), 3.90 (s, 2H), 2.49 (s, 3H), 2.27 (d, J=1.9 Hz, 3H), 1.26 (s, 6H).

### Example 653: N-[6-[5-(4-hydroxycyclohexoxy)-2-methyl-4-pyridyl]imidazo[1,2-a]pyrazin-2-yl]cyclopropanecarboxamide

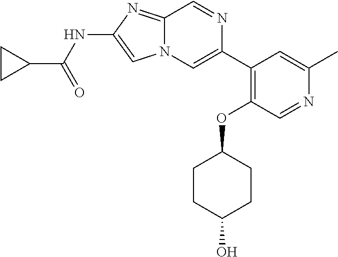 [SMILES: Cc1cc(-c2cn3cc(NC(=O)C4CC4)nc3cn2)c(O[C@H]2CC[C@H](O)CC2)cn1]

The title compound was prepared in the similar manner of Example 505 except trans-cyclohexane-1,4-diol was used instead of tert-butyl (1R,5S,7s)-7-hydroxy-3-oxa-9-azabicyclo[3.3.1]nonane-9-carboxylate (121 mg, 24%). MS (ESI): mass calcd. for C₂₂H₂₅N₅O₃, 407.2. m/z found, 408.2 [M+H]⁺. ¹H NMR (400 MHz, CD₃OD) δ 9.06 (d, J=1.4 Hz, 1H), 8.82 (dd, J=1.5, 0.7 Hz, 1H), 8.25-8.13 (m, 2H), 7.82 (s, 1H), 4.44 (dq, J=8.8, 4.4, 3.9 Hz, 1H), 3.58 (td, J=9.1, 4.4 Hz, 1H), 2.44 (s, 3H), 2.18-2.00 (m, 2H), 1.95-1.74 (m, 3H), 1.66-1.47 (m, 2H), 1.46-1.25 (m, 2H), 0.92 (dt, J=4.6, 3.1 Hz, 2H), 0.84 (tdd, J=7.6, 5.6, 2.6 Hz, 2H).

### Example 654: N-[6-[2-methyl-5-(1-methylazetidin-3-yl)oxy-4-pyridyl]imidazo[1,2-a]pyrazin-2-yl]cyclopropanecarboxamide

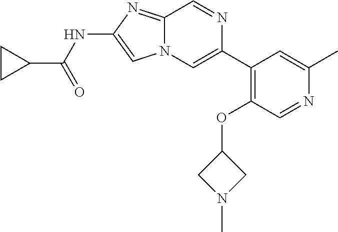 [SMILES: Cc1cc(-c2cn3cc(NC(=O)C4CC4)nc3cn2)c(OC2CN(C)C2)cn1]

The title compound was prepared in the similar manner of Example 505 except 1-methylazetidin-3-ol was used instead of tert-butyl (1R,5S,7s)-7-hydroxy-3-oxa-9-azabicyclo[3.3.1]nonane-9-carboxylate (12.8 mg, 13%). MS (ESI): mass calcd. for C₂₀H₂₂N₆O₂, 378.2. m/z found, 379.2 [M+H]⁺. ¹H NMR (500 MHz, CD₃OD) δ 9.30 (d, J=1.5 Hz, 1H), 9.00-8.87 (m, 1H), 8.35 (s, 1H), 7.95 (d, J=1.2 Hz, 2H), 5.04 (tt, J=5.9, 4.2 Hz, 1H), 3.92-3.72 (m, 2H), 3.52-3.38 (m, 2H), 2.53 (s, 3H), 2.44 (s, 3H), 1.92 (tt, J=7.9, 4.6 Hz, 1H), 1.05-0.97 (m, 2H), 0.95-0.87 (m, 2H).

### Example 655: N-[6-[5-(4-hydroxycyclohexoxy)-2-methyl-4-pyridyl]imidazo[1,2-a]pyrazin-2-yl]cyclopropanecarboxamide

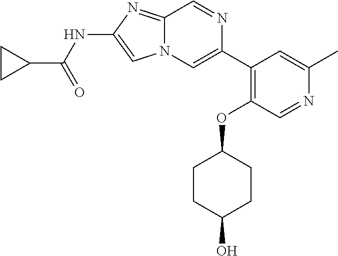 [SMILES: Cc1cc(-c2cn3cc(NC(=O)C4CC4)nc3cn2)c(O[C@H]2CC[C@@H](O)CC2)cn1]

The title compound was prepared in the similar manner of Example 505 except cis-cyclohexyldiol was used instead of tert-butyl (1R,5S,7s)-7-hydroxy-3-oxa-9-azabicyclo[3.3.1]nonane-9-carboxylate (29.2 mg, 19%). MS (ESI): mass calcd. for C₂₂H₂₅N₅O₃, 407.2. m/z found, 408.2 [M+H]⁺. ¹H NMR (400 MHz, CD₃OD) δ 9.22 (d, J=1.4 Hz, 1H), 8.95 (s, 1H), 8.32 (d, J=13.4 Hz, 2H), 7.93 (s, 1H), 4.72 (s, 1H), 3.72 (dt, J=8.2, 4.2 Hz, 1H), 2.56 (s, 3H), 2.08 (d, J=8.7 Hz, 2H), 2.05 (s, 1H), 1.93 (td, J=7.6, 3.5 Hz, 1H), 1.86-1.68 (m, 4H), 1.68-1.55 (m, 2H), 1.03 (dt, J=5.8, 3.1 Hz, 2H), 0.95 (dt, J=8.0, 3.1 Hz, 2H).

### Example 656: N-[5-[2-cyano-5-[[(1R,5S,7s)-9-methyl-3-oxa-9-azabicyclo[3.3.1]nonan-7-yl]oxy]-4-pyridyl]pyrazolo[1,5-a]pyridin-2-yl]cyclopropanecarboxamide

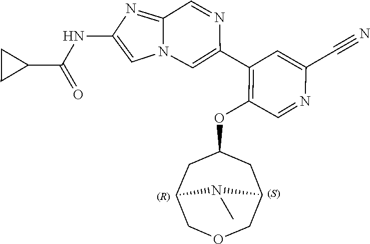

The title compound was prepared in the similar manner of Example 505 except N-(5-(2-cyano-5-fluoropyridin-4-yl)pyrazolo[1,5-a]pyridin-2-yl)cyclopropanecarboxamide was used instead of N-(6-(5-fluoro-2-methylpyridin-4-yl)imidazo[1,2-a]pyrazin-2-yl)cyclopropanecarboxamide and an additional reductive amination step was performed as follows: H(CHO)n (30 mg, 0.34 mmol) was added to a solution consisting of N-(5-(5-((1R,5S,7s)-3-oxa-9-azabicyclo[3.3.1]nonan-7-yloxy)-2-cyanopyridin-4-yl)pyrazolo[1,5-a]pyridin-2-yl)cyclopropanecarboxamide (100 mg, 0.225 mmol) and MeOH (10 mL). The reaction mixture was stirred at room-temperature for 30 min, and then NaBH₃CN (42.4 mg, 0.675 mmol) was added to the reaction mixture. The resultant mixture was stirred at room-temperature for 1 hour. The crude mixture was purified via preparative HPLC to afford (21.1 mg, 20%). MS (ESI): mass calcd. for C₂₅H₂₆N₆O₃, 458.2. m/z found, 459.3 [M+H]⁺. ¹H NMR (400 MHz, DMSO-d₆) δ 11.12 (s, 1H), 8.56 (d, J=7.2 Hz, 1H), 8.50 (d, J=0.8 Hz, 1H), 8.43-8.39 (m, 1H), 8.22 (d, J=1.6 Hz, 1H), 7.33 (d, J=7.2 Hz, 1H), 6.86 (s, 1H), 5.04-4.93 (m, 1H), 3.80 (d, J=10.4 Hz, 2H), 3.56 (d, J=10.8 Hz, 2H), 2.68-2.60 (m, 2H), 2.49-2.43 (m, 2H), 2.41 (s, 3H), 1.99-1.85 (m, 1H), 1.73-1.60 (m, 2H), 0.89-0.77 (m, 4H)

### Example 657: 4-[[6-methyl-4-[2-[(6-methylpyrazin-2-yl)amino]imidazo[1,2-a]pyrazin-6-yl]-3-pyridyl]oxy]cyclohexanol

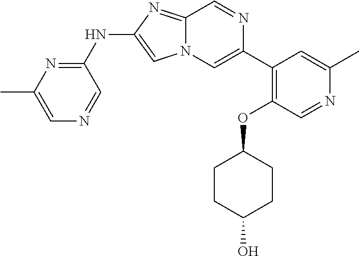

The title compound was prepared in the similar manner of Example 320 except 2-chloro-6-methylpyrazine was used instead of 2-chloro-5-methylpyrazine in step B and trans-cyclohexane-1,4-diol instead of endo-tert-butyl (1R,5S,7s)-7-hydroxy-3-oxa-9-azabicyclo[3.3.1]nonane-9-carboxylate in step C.MS (ESI): mass calcd. for C₂₃H₂₅N₇O₂, 431.2. m/z found, 432.2 [M+H]⁺. ¹H NMR (400 MHz, DMSO-d₆) δ 10.45 (s, 1H), 9.22 (d, J=1.4 Hz, 1H), 8.96 (dd, J=1.5, 0.7 Hz, 1H), 8.51 (d, J=0.7 Hz, 1H), 8.40 (s, 1H), 8.34 (s, 1H), 7.93 (s, 1H), 7.82 (s, 1H), 4.52 (dt, J=9.2, 4.9 Hz, 2H), 3.54 (dt, J=9.2, 4.9 Hz, 1H), 2.47 (s, 3H), 2.04 (d, J=5.6 Hz, 2H), 1.80 (dd, J=13.0, 3.9 Hz, 2H), 1.65-1.51 (m, 2H), 1.33 (q, J=12.9 Hz, 2H).

### Example 658: N-(2,6-dimethylpyrimidin-4-yl)-5-[2-ethoxy-5-[[(2S)-morpholin-2-yl]methoxy]-4-pyridyl]pyrazolo[1,5-a]pyridin-2-amine

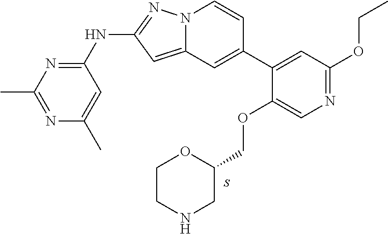

The title compound was prepared using methods analogous to those described in Example 501. MS (ESI): mass calcd. for C₂₅H₂₉N₇O₃, 475.2. m/z found, 476.2 [M+H]⁺. ¹H NMR (500 MHz, CD₃OD) δ 8.34 (dt, J=7.1, 0.9 Hz, 1H), 7.80 (s, 1H), 7.73 (dd, J=1.9, 0.9 Hz, 1H), 6.99-6.84 (m, 2H), 6.78 (d, J=4.1 Hz, 2H), 4.21 (q, J=7.1 Hz, 2H), 3.99-3.87 (m, 2H), 3.82-3.74 (m, 1H), 3.68 (ddd, J=11.7, 5.7, 3.6 Hz, 1H), 3.51 (ddd, J=11.5, 9.3, 5.0 Hz, 1H), 2.77 (dd, J=12.5, 2.4 Hz, 1H), 2.70-2.62 (m, 2H), 2.56 (dd, J=12.5, 10.6 Hz, 1H), 2.44 (s, 3H), 2.30-2.23 (m, 3H), 1.28 (t, J=7.1 Hz, 3H).

### Example 659: 3-[[4-[2-[(2,6-dimethylpyrimidin-4-yl)amino]pyrazolo[1,5-a]pyridin-5-yl]-6-(2,2,2-trifluoroethoxy)-3-pyridyl]oxy]-2,2-dimethyl-propanenitrile

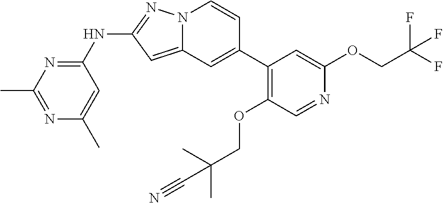 [SMILES: Cc1cc(Nc2cc3cc(-c4cc(OCC(F)(F)F)ncc4OCC(C)(C)C#N)ccn3n2)nc(C)n1]

The title compound was prepared using a coupling method similar to that described in Example 501. MS (ESI): mass calcd. for C₂₅H₂₄F₃N₇O₂, 511.2. m/z found, 512.2 [M+H]⁺. ¹H NMR (400 MHz, DMSO-d₆) δ 10.25 (s, 1H), 8.60 (d, J=7.2 Hz, 1H), 8.07 (s, 1H), 7.91 (s, 1H), 7.16 (s, 1H), 7.09-6.98 (m, 2H), 6.86 (br. s., 1H), 5.00 (q, J=9.2 Hz, 2H), 4.16 (s, 2H), 2.45 (s, 3H), 2.30 (s, 3H), 1.35 (s, 6H). ¹⁹F NMR (376 MHz, DMSO-d₆) δ −72.30 (t, J=9.0 Hz, 3F)

### Example 660: 3-[[6-(2,2-difluoroethoxy)-4-[2-[(2,6-dimethylpyrimidin-4-yl)amino]pyrazolo[1,5-a]pyridin-5-yl]-3-pyridyl]oxy]-2,2-dimethyl-propanenitrile

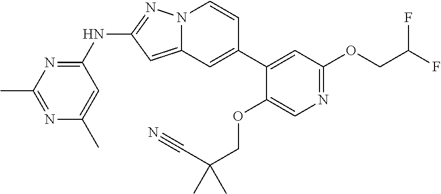 [SMILES: Cc1cc(Nc2cc3cc(-c4cc(OCC(F)F)ncc4OCC(C)(C)C#N)ccn3n2)nc(C)n1]

The title compound was prepared using a coupling method similar to that described in Example 501. MS (ESI): mass calcd. for C₂₅H₂₅F₂N₇O₂, 493.2. m/z found, 494.2 [M+H]⁺. ¹H NMR (400 MHz, DMSO-d₆) δ10.24 (s, 1H), 8.60 (d, J=7.2 Hz, 1H), 8.06 (s, 1H), 7.89 (d, J=0.8 Hz, 1H), 7.09 (s, 1H), 7.07-7.00 (m, 2H), 6.86 (s, 1H), 6.58-6.24 (m, 1H), 4.57 (dt, J=3.6, 15.2 Hz, 2H), 4.14 (s, 2H), 2.45 (s, 3H), 2.30 (s, 3H), 1.35 (s, 6H) 19F NMR (376 MHz, DMSO-d₆) δ−125.57 (td, J=15.0, 54.9 Hz, 2F)

### Example 661: 1-[[4-[2-[(2,6-dimethylpyrimidin-4-yl)amino]pyrazolo[1,5-a]pyridin-5-yl]-6-(2,2,2-trifluoroethoxy)-3-pyridyl]oxy]-2-methyl-propan-2-ol

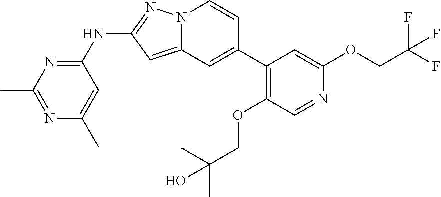 [SMILES: Cc1cc(Nc2cc3cc(-c4cc(OCC(F)(F)F)ncc4OCC(C)(C)O)ccn3n2)nc(C)n1]

The title compound was prepared using methods analogous to those described in Example 501. MS (ESI): mass calcd. for C₂₄H₂₅F₃N₆O₃, 502.2. m/z found, 503.2 [M+H]⁺. ¹H NMR (400 MHz, DMSO-d₆) δ10.22 (s, 1H), 8.59 (d, J=7.6 Hz, 1H), 8.03 (s, 1H), 7.91 (d, J=1.2 Hz, 1H), 7.12 (s, 1H), 7.09 (dd, J=2.0, 7.2 Hz, 1H), 7.03 (br. s., 1H), 6.84 (br. s., 1H), 4.98 (q, J=8.8 Hz, 2H), 4.65 (s, 1H), 3.84 (s, 2H), 2.46 (s, 3H), 2.30 (s, 3H), 1.15 (s, 6H) ¹⁹F NMR (376 MHz, DMSO-d₆) δ−72.32 (t, J=9.4 Hz, 3F).

### Example 662: N-(2,6-dimethylpyrimidin-4-yl)-5-[5-[[(2S)-morpholin-2-yl]methoxy]-2-(2,2,2-trifluoroethoxy)-4-pyridyl]pyrazolo[1,5-a]pyridin-2-amine

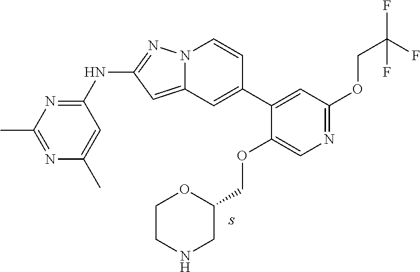

The title compound was prepared using methods analogous to those described in Example 501. MS (ESI): mass calcd. for C₂₅H₂₆F₃N₇O₃, 529.2. m/z found, 530.2 [M+H]⁺. ¹H NMR (400 MHz, CD₃OD) δ 8.33 (dt, J=7.3, 1.0 Hz, 1H), 7.85 (s, 1H), 7.75 (dd, J=2.0, 0.9 Hz, 1H), 6.98-6.92 (m, 2H), 6.91 (s, 1H), 6.78 (s, 1H), 4.72 (d, J=8.8 Hz, 2H), 4.01-3.91 (m, 2H), 3.83-3.76 (m, 1H), 3.74-3.64 (m, 1H), 3.52 (ddd, J=11.5, 8.8, 5.3 Hz, 1H), 2.78 (dd, J=12.6, 2.4 Hz, 1H), 2.68 (dd, J=8.7, 3.0 Hz, 2H), 2.58 (dd, J=12.5, 10.6 Hz, 1H), 2.43 (s, 3H), 2.28 (s, 3H).

### Example 663: 3-[[4-[2-[(2,6-dimethylpyrimidin-4-yl)amino]pyrazolo[1,5-a]pyridin-5-yl]-6-isopropoxy-3-pyridyl]oxy]-2,2-dimethyl-propanenitrile

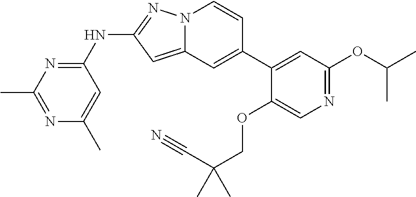 [SMILES: Cc1cc(Nc2cc3cc(-c4cc(OC(C)C)ncc4OCC(C)(C)C#N)ccn3n2)nc(C)n1]

The title compound was prepared using methods analogous to those described in Example 501. MS (ESI): mass calcd. for C₂₆H₂₉N₇O₂, 471.2. m/z found, 472.2 [M+H]⁺. ¹H NMR (400 MHz, DMSO-d₆) δ 10.23 (s, 1H), 8.58 (d, J=7.2 Hz, 1H), 8.02 (s, 1H), 7.86 (s, 1H), 7.08-6.99 (m, 2H), 6.87 (s, 1H), 6.83 (br. s., 1H), 5.27-5.16 (m, 1H), 4.10 (s, 2H), 2.45 (s, 3H), 2.30 (s, 3H), 1.34 (s, 6H), 1.31 (d, J=6.4 Hz, 6H).

### Example 664: 4-[[4-[2-[(2,6-dimethylpyrimidin-4-yl)amino]pyrazolo[1,5-a]pyridin-5-yl]-6-(2,2,2-trifluoroethoxy)-3-pyridyl]oxy]cyclohexanol

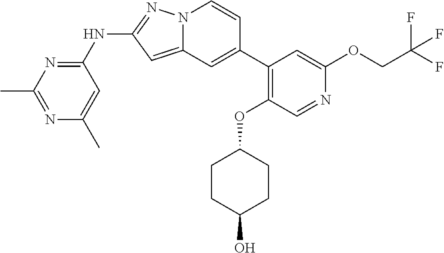 [SMILES: Cc1cc(Nc2cc3cc(-c4cc(OCC(F)(F)F)ncc4O[C@H]4CC[C@H](O)CC4)ccn3n2)nc(C)n1]

Step A: 5-(5-(((1r,4r)-4-((tert-butyldimethylsilyl)oxy)cyclohexyl)oxy)-2-chloropyridin-4-yl)-N-(2,6-dimethylpyrimidin-4-yl)pyrazolo[1,5-a]pyridin-2-amine. The title compound was prepared using methods analogous to those described in Example 198 and using 5-(2-chloro-5-fluoropyridin-4-yl)-N-(2,6-dimethylpyrimidin-4-yl)pyrazolo[1,5-a]pyridin-2-amine instead of Intermediate 15 and using (1r,4r)-4-((tert-butyldimethylsilyl)oxy)cyclohexanol instead of trans-1,4-cyclohexanediol.

Step B: 5-(5-4(1r,4r)-4-((tert-butyldimethylsilyl)oxy)cyclohexyl)oxy)-2-(2,2,2-trifluoroethoxy)pyridin-4-yl)-N-(2,6-dimethylpyrimidin-4-yl)pyrazolo[1,5-a]pyridin-2-amine. The title compound was prepared using a coupling method similar to that described in Example 501.

Step C: 4-[[4-[2-[(2,6-dimethylpyrimidin-4-yl)amino]pyrazolo[1,5-a]pyridin-5-yl]-6-(2,2,2-trifluoroethoxy)-3-pyridyl]oxy]cyclohexanol. A mixture consisting of H₂SO₄ (0.8 mL, 1.6 mmol) and 5-(5-(((1r,4r)-4-((tert-butyldimethylsilyl)oxy)cyclohexyl)oxy)-2-(2,2,2-trifluoroethoxy)pyridin-4-yl)-N-(2,6-dimethylpyrimidin-4-yl)pyrazolo[1,5-a]pyridin-2-amine (40 mg, 0.062 mmol) and MeCN (0.5 mL) was stirred at room-temperature for 3 h. The reaction mixture was purified by preparative HPLC using a Phenomenex Gemini-NX C18 150×30 mm×5 μm (eluent: 42% to 70% (v/v) water (0.05% NH₃H₂O)-ACN to afford pure product. The product was suspended in water (10 mL), the mixture frozen using dry ice/ethyl alcohol, and then lyophilized to dryness to afford the title compound (21.9 mg, 66%) as a white solid. MS (ESI): mass calcd. for C₂₆H₂₇F₃N₆O₃, 528.1. m/z found, 529.1 [M+H]⁺. ¹H NMR (400 MHz, DMSO-d₆) δ 10.22 (s, 1H), 8.58 (d, J=7.2 Hz, 1H), 8.09 (s, 1H), 7.83 (d, J=0.8 Hz, 1H), 7.09 (s, 1H), 7.05-6.98 (m, 2H), 6.87 (br. s., 1H), 4.97 (q, J=9.2 Hz, 2H), 4.52 (d, J=4.0 Hz, 1H), 4.35-4.27 (m, 1H), 3.52-3.43 (m, 1H), 2.46 (s, 3H), 2.30 (s, 3H), 2.01-1.92 (m, 2H), 1.78-1.66 (m, 2H), 1.44-1.34 (m, 2H), 1.30-1.23 (m, 2H). ¹⁹F NMR (376 MHz, DMSO-d₆) δ −72.29 (s, 3F)

### Example 665: N-[5-[5-(2-cyano-2-methyl-propoxy)-2-(difluoromethoxy)-4-pyridyl]pyrazolo[1,5-a]pyridin-2-yl]cyclopropanecarboxamide

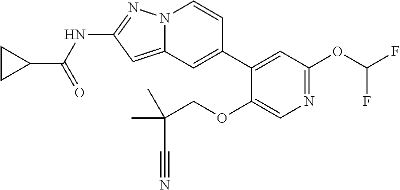 [SMILES: CC(C)(C#N)COc1cnc(OC(F)F)cc1-c1ccn2nc(NC(=O)C3CC3)cc2c1]

The title compound was prepared using methods analogous to those described in Intermediate 27 except Intermediate 3 was used instead of Intermediate 14. MS (ESI): mass calcd. for C₂₂H₂₁F₂N₅O₃, 441.2. m/z found, 442.2 [M+H]⁺. ¹H NMR (500 MHz, CD₃OD) δ 8.33 (dt, J=7.3, 0.9 Hz, 1H), 7.94 (s, 1H), 7.77 (dd, J=2.0, 0.9 Hz, 1H), 7.39 (t, J=73.3 Hz, 1H), 7.03 (d, J=0.4 Hz, 1H), 7.00 (dd, J=7.3, 2.0 Hz, 1H), 6.83 (s, 1H), 4.02 (s, 2H), 1.77 (tt, J=8.1, 4.5 Hz, 1H), 1.30 (s, 6H), 0.94-0.85 (m, 2H), 0.85-0.71 (m, 2H).

### Example 666: N-[5-[2-chloro-5-(2-cyano-2-methyl-propoxy)-4-pyridyl]pyrazolo[1,5-a]pyridin-2-yl]cyclopropanecarboxamide

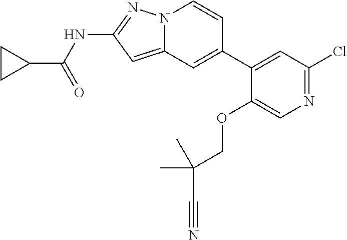 [SMILES: CC(C)(C#N)COc1cnc(Cl)cc1-c1ccn2nc(NC(=O)C3CC3)cc2c1]

The title compound was prepared using methods analogous to those described in Example 262. MS (ESI): mass calcd. for C₂₁H₂₀ClN₅O₂, 409.9. m/z found, 432.1 [M+H]⁺. ¹H NMR (400 MHz, DMSO-d₆) δ 11.13 (s, 1H), 8.61 (dt, J=7.3, 0.9 Hz, 1H), 8.34 (s, 1H), 7.96 (dd, J=2.0, 0.9 Hz, 1H), 7.67 (s, 1H), 7.11 (dd, J=7.2, 2.1 Hz, 1H), 6.91 (d, J=0.8 Hz, 1H), 4.27 (s, 2H), 2.00-1.87 (m, 1H), 1.37 (s, 6H), 0.90-0.75 (m, 4H).

### Example 667: 3-[[6-(2,2-difluoroethylamino)-4-[2-[(2,6-dimethylpyrimidin-4-yl)amino]pyrazolo[1,5-a]pyridin-5-yl]-3-pyridyl]oxy]-2,2-dimethyl-propanenitrile

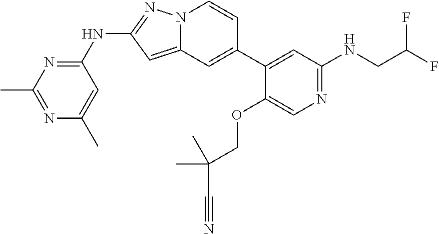 [SMILES: Cc1cc(Nc2cc3cc(-c4cc(NCC(F)F)ncc4OCC(C)(C)C#N)ccn3n2)nc(C)n1]

Step A: 3-((6-chloro-4-(2-((2,6-dimethylpyrimidin-4-yl)amino)pyrazolo[1,5-a]pyridin-5-yl)pyridin-3-yl)oxy)-2,2-dimethylpropanenitrile. The title compound was prepared using methods analogous to those described in Example 666.

Step B: The title compound was prepared using methods analogous to those described in Example 501 and using 3-((6-chloro-4-(2-((2,6-dimethylpyrimidin-4-yl)amino)pyrazolo[1,5-a]pyridin-5-yl)pyridin-3-yl)oxy)-2,2-dimethylpropanenitrile, 2,2-difluoroethanamine, Pd₂(dba)₃, Xantphos, NaOtBu, and 1,4-dioxane instead of tert-butyl (S)-2-(((6-chloro-4-(2-((2,6-dimethylpyrimidin-4-yl)amino)pyrazolo[1,5-a]pyridin-5-yl)pyridin-3-yl)oxy)methyl)morpholine-4-carboxylate, methylamine, JosiPhos pre-catalyst gen. 3, Cs₂CO₃, and toluene. MS (ESI): mass calcd. for C₂₅H₂₆F₂N80, 492.2. m/z found, 493.2 [M+H]⁺. ¹H NMR (400 MHz, CDCl₃) δ 8.40 (d, J=7.2 Hz, 1H), 7.87 (s, 1H), 7.65 (d, J=0.8 Hz, 1H), 7.58 (s, 1H), 7.11 (s, 1H), 6.97 (dd, J=2.0, 7.2 Hz, 1H), 6.57 (s, 1H), 6.54 (s, 1H), 6.16-5.83 (m, 1H), 4.60 (t, J=6.4 Hz, 1H), 3.84 (s, 2H), 3.83-3.73 (m, 2H), 2.60 (s, 3H), 2.45 (s, 3H), 1.37 (s, 6H). ¹⁹F NMR (376 MHz, CDCl₃) δ −123.16 (s, 2F)

### Example 668: 3-[[4-[2-[(2,6-dimethylpyrimidin-4-yl)amino]pyrazolo[1,5-a]pyridin-5-yl]-6-(methoxymethyl)-3-pyridyl]oxy]-2,2-dimethyl-propanenitrile

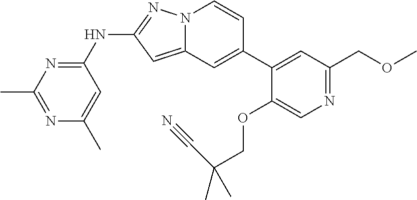

The title compound was prepared using a coupling method similar to that described in Example 441, Step B, and using 3-((6-chloro-4-(2-((2,6-dimethylpyrimidin-4-yl)amino)pyrazolo[1,5-a]pyridin-5-yl)pyridin-3-yl)oxy)-2,2-dimethylpropanenitrile (Example 667 Step A), tributyl(methoxymethyl)stannane, Pd(PPh₃)₄, and NMP instead of (R)—N-(5-(2-chloro-5-((l-methylpyrrolidin-3-yl)methoxy)pyridin-4-yl)pyrazolo[1,5-a]pyridin-2-yl)cyclopropanecarboxamide, tributyl(1-propynyl)tin, bis(triphenylphosphine)palladium(ii) dichloride, and 1,4-dioxane. MS (ESI): mass calcd. for C₂₅H₂₇N₇O₂, 457.2. m/z found, 458.2 [M+H]⁺. ¹H NMR (400 MHz, CDCl₃) δ 8.41 (d, J=7.2 Hz, 1H), 8.28 (s, 1H), 7.73 (s, 1H), 7.50 (s, 1H), 7.42 (s, 1H), 7.13 (s, 1H), 7.04 (dd, J=1.6, 7.2 Hz, 1H), 6.58 (s, 1H), 4.60 (s, 2H), 4.04 (s, 2H), 3.51 (s, 3H), 2.60 (s, 3H), 2.46 (s, 3H), 1.42 (s, 6H).

### Example 669: N-[5-[5-(2-cyano-2-methyl-propoxy)-2-(2,2,2-trifluoroethoxy)-4-pyridyl]pyrazolo[1,5-a]pyridin-2-yl]cyclopropanecarboxamide

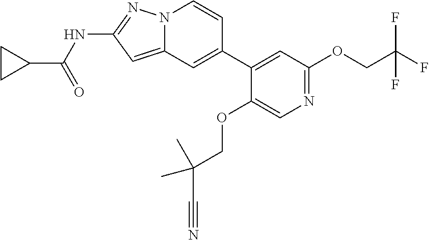 [SMILES: CC(C)(C#N)COc1cnc(OCC(F)(F)F)cc1-c1ccn2nc(NC(=O)C3CC3)cc2c1]

The title compound was prepared using a coupling method similar to that described in Example 501. MS (ESI): mass calcd. for C₂₃H₂₂F₃N₅O₃, 473.2. m/z found, 474.2 [M+H]⁺. ¹H NMR (400 MHz, CD₃OD) δ 8.44 (dt, J=7.2, 0.9 Hz, 1H), 8.00 (s, 1H), 7.86 (dd, J=2.0, 0.9 Hz, 1H), 7.11 (dd, J=7.3, 2.0 Hz, 1H), 7.06 (s, 1H), 6.94 (s, 1H), 4.90 (s, 2H), 4.09 (s, 2H), 1.89 (tt, J=8.1, 4.5 Hz, 1H), 1.02 (dt, J=4.6, 3.1 Hz, 2H), 0.98-0.86 (m, 2H).

### Example 670: 3-[[4-[2-[(2,6-dimethylpyrimidin-4-yl)amino]pyrazolo[1,5-a]pyridin-5-yl]-6-prop-1-ynyl-3-pyridyl]oxy]-2,2-dimethyl-propanenitrile

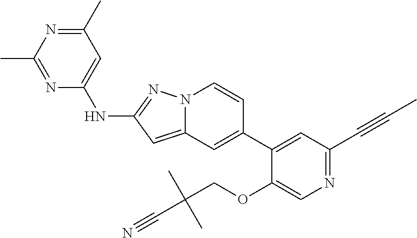 [SMILES: CC#Cc1cc(-c2ccn3nc(Nc4cc(C)nc(C)n4)cc3c2)c(OCC(C)(C)C#N)cn1]

The title compound was prepared using a similar method to that described in Example 671. MS (ESI): mass calcd. for C₂₆H₂₅N₇O, 451.2. m/z found, 452.2 [M+H]⁺. ¹H NMR (400 MHz, DMSO-d₆) δ 10.22 (s, 1H), 8.62-8.57 (m, 1H), 8.45 (s, 1H), 7.92-7.86 (m, 1H), 7.58 (s, 1H), 7.09-6.98 (m, 2H), 6.85 (s, 1H), 4.28 (s, 2H), 2.45 (s, 3H), 2.32-2.28 (m, 3H), 2.08 (s, 3H), 1.36 (s, 6H).

### Example 671: (2S,3R)-2-[[4-[2-[(2,6-dimethylpyrimidin-4-yl)amino]pyrazolo[1,5-a]pyridin-5-yl]-6-prop-1-ynyl-3-pyridyl]oxymethyl]tetrahydrofuran-3-ol

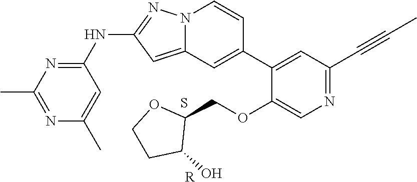

Step A: (2S,3R)-2-(((6-chloro-4-(2-((2,6-dimethylpyrimidin-4-yl)amino)pyrazolo[1,5-a]pyridin-5-yl)pyridin-3-yl)oxy)methyl)tetrahydrofuran-3-ol. To a solution of Intermediate 26 (1.00 g, 2.73 mmol, 1.00 equiv), ((2S,3R)-3-hydroxytetrahydrofuran-2-yl)methyl 4-methylbenzenesulfonate (0.74 g, 2.73 mmol, 1.00 equiv), Cs₂CO₃ (2.66 g. 8.18 mmol, 3.00 equiv) in DMA (6.8 mL) was stirred at 50 C for 16 hr. The reaction mixture was then cooled to room temperature and diluted with EtOAc, then filtered through celite. The organics were then washed with water and then brine. The aqueous was back-extracted with 10% IPA/chloroform (100 mL) and then 20% IPA/chloroform (2×20 mL). The combined organics were dried over Na₂SO₄, filtered and concentrated in vacuo. The crude oil was diluted with diethyl ether (40 mL) and the resulting suspension was filtered to give the title compound as a tan solid (798 mg, 1.71 mmol, 62% yield).

Step B: (2S,3R)-2-[[4-[2-[(2,6-dimethylpyrimidin-4-yl)amino]pyrazolo[1,5-a]pyridin-5-yl]-6-prop-1-ynyl-3-pyridyl]oxymethyl]tetrahydrofuran-3-ol. The title compound was prepared using a coupling method similar to that described in Example 441, Step B. MS (ESI): mass calcd. for C₂₆H₂₆N₆O₃, 470.2. m/z found, 471.2 [M+H]⁺. ¹H NMR (400 MHz, DMSO-d₆) δ10.20 (s, 1H), 8.61-8.57 (m, 1H), 8.43 (s, 1H), 7.90-7.84 (m, 1H), 7.53 (s, 1H), 7.08-6.95 (m, 2H), 6.86 (s, 1H), 5.06 (d, J=4.3 Hz, 1H), 4.28-4.12 (m, 3H), 3.90-3.75 (m, 3H), 2.46 (s, 3H), 2.32-2.27 (m, 3H), 2.07 (s, 3H), 1.98-1.87 (m, 1H), 1.75-1.63 (m, 1H).

### Example 672: 3-[[6-(cyanomethoxy)-4-[2-[(2,6-dimethylpyrimidin-4-yl)amino]pyrazolo[1,5-a]pyridin-5-yl]-3-pyridyl]oxy]-2,2-dimethyl-propanenitrile

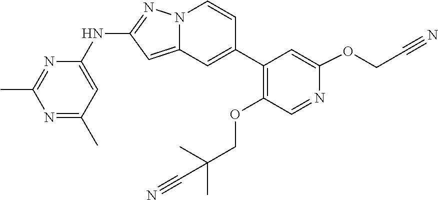 [SMILES: Cc1cc(Nc2cc3cc(-c4cc(OCC#N)ncc4OCC(C)(C)C#N)ccn3n2)nc(C)n1]

Step A: 3-((6-(cyanomethoxy)-4-iodopyridin-3-yl)oxy)-2,2-dimethylpropanenitrile. A mixture consisting 3-((6-hydroxy-4-iodopyridin-3-yl)oxy)-2,2-dimethylpropanenitrile (Example 329, Step B, 500 mg, 1.57 mmol), 2-chloroacetonitrile (142 mg, 1.87 mmol), Ag₂CO₃ (867 mg, 3.14 mmol), and NMP (12 mL) was stirred at 120° C. for 3 h. The reaction mixture was concentrated under reduced pressure to give the crude product which was purified by FCC (eluent: petroleum ether:ethyl acetate=1:0 to 1:1) to afford the title compound (200 mg, 29%) as a yellow oil.

Step B: 3-[[6-(cyanomethoxy)-4-[2-[(2,6-dimethylpyrimidin-4-yl)amino]pyrazolo[1,5-a]pyridin-5-yl]-3-pyridyl]oxy]-2,2-dimethyl-propanenitrile. The title compound was prepared using the method analogous to that described in Example 329, Step D, and using 3-((6-(cyanomethoxy)-4-iodopyridin-3-yl)oxy)-2,2-dimethylpropanenitrile instead of 3-((6-(difluoromethoxy)-4-iodopyridin-3-yl)oxy)-2,2-dimethylpropanenitrile. MS (ESI): mass calcd. for C₂₅H₂₄N₈O₂, 468.2. m/z found, 469.3 [M+H]⁺. ¹H NMR (400 MHz, DMSO-d₆) δ 10.23 (s, 1H), 8.61 (d, J=6.4 Hz, 1H), 8.12 (s, 1H), 7.90 (s, 1H), 7.15 (s, 1H), 7.09-6.97 (m, 2H), 6.87 (s, 1H), 5.22 (s, 2H), 4.18 (s, 2H), 2.46 (s, 3H), 2.31 (s, 3H), 1.35 (s, 6H).

### Example 673: 3-[[4-[2-[(2,6-dimethylpyrimidin-4-yl)amino]pyrazolo[1,5-a]pyridin-5-yl]-6-(2-fluoroethoxy)-3-pyridyl]oxy]-2,2-dimethyl-propanenitrile

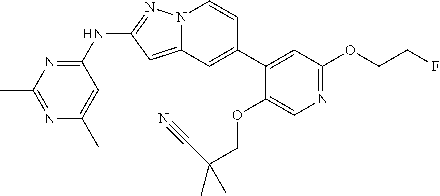 [SMILES: Cc1cc(Nc2cc3cc(-c4cc(OCCF)ncc4OCC(C)(C)C#N)ccn3n2)nc(C)n1]

The title compound was prepared using a coupling method similar to that described in Example 501. MS (ESI): mass calcd. for C₂₅H₂₆FN₇O₂, 475.1. m/z found, 476.1 [M+H]⁺. ¹H NMR (400 MHz, DMSO-d₆) δ 10.23 (s, 1H), 8.59 (d, J=7.6 Hz, 1H), 8.04 (s, 1H), 7.88 (d, J=1.2 Hz, 1H), 7.07-6.99 (m, 3H), 6.85 (s, 1H), 4.87-4.66 (m, 2H), 4.60-4.41 (m, 2H), 4.12 (s, 2H), 2.45 (s, 3H), 2.30 (s, 3H), 1.35 (s, 6H). ¹⁹F NMR (376 MHz, DMSO-d₆) δ −223.29 (m, 1F).

### Example 674: 3-[[6-hydroxy-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile

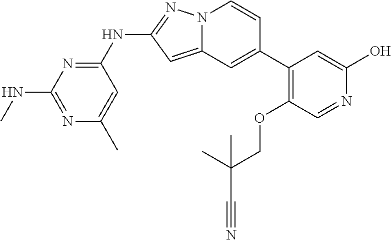 [SMILES: CNc1nc(C)cc(Nc2cc3cc(-c4cc(O)ncc4OCC(C)(C)C#N)ccn3n2)n1]

The title compound was isolated as a by-product from the synthesis of Example 675. MS (ESI): mass calcd. for C₂₃H₂₄N₈O₂, 444.2. m/z found, 445.2 [M+H]⁺. ¹H NMR (500 MHz, DMSO-d₆) δ 9.84 (s, 1H), 8.51 (dt, J=7.2, 0.9 Hz, 1H), 7.76 (s, 1H), 7.31 (s, 1H), 7.05 (s, 1H), 6.91 (dd, J=7.2, 2.0 Hz, 1H), 6.60 (s, 1H), 6.52 (s, 1H), 6.22 (s, 1H), 3.90 (s, 2H), 2.83 (d, J=4.8 Hz, 3H), 2.13 (s, 3H), 1.29 (s, 6H).

### Example 675: 3-[[6-chloro-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile

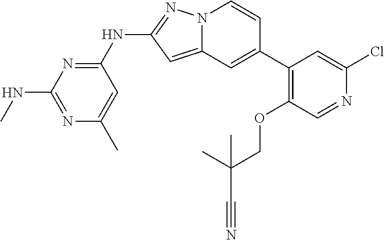 [SMILES: CNc1nc(C)cc(Nc2cc3cc(-c4cc(Cl)ncc4OCC(C)(C)C#N)ccn3n2)n1]

Step A: 3-((4-(2-aminopyrazolo[1,5-a]pyridin-5-yl)-6-chloropyridin-3-yl)oxy)-2,2-dimethylpropanenitrile. The title compound was prepared using methods analogous to those described in Intermediate 25, Step C, and using 3-((6-chloro-4-iodopyridin-3-yl)oxy)-2,2-dimethylpropanenitrile instead of (2R,3S)-2-(((4-bromo-6-methylpyridin-3-yl)oxy)methyl)tetrahydrofuran-3-ol.

Step B: 3-[[6-chloro-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile. The title compound was prepared using methods analogous to those described in Example 116, Step A, and using 4-chloro-n,6-dimethylpyrimidin-2-amine and 3-((4-(2-aminopyrazolo[1,5-a]pyridin-5-yl)-6-chloropyridin-3-yl)oxy)-2,2-dimethylpropanenitrile instead of 4-bromo-1-(difluoromethyl)-1H-pyrazole and Intermediate 9. MS (ESI): mass calcd. for C₂₃H₂₃ClN₈O, 462.2. m/z found, 463.2 [M+H]⁺. ¹H NMR (500 MHz, DMSO-d₆) δ 9.88 (s, 1H), 8.57 (d, J=7.2 Hz, 1H), 8.32 (s, 1H), 7.91 (s, 1H), 7.67 (s, 1H), 7.09 (s, 1H), 7.04 (dd, J=7.2, 2.0 Hz, 1H), 6.61 (s, 1H), 6.21 (s, 1H), 4.26 (s, 2H), 2.84 (d, J=4.8 Hz, 3H), 2.13 (s, 3H), 1.36 (s, 6H).

### Example 676: 3-[[6-(difluoromethoxy)-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile

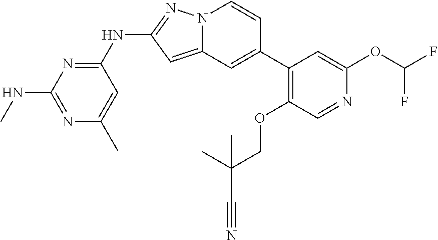 [SMILES: CNc1nc(C)cc(Nc2cc3cc(-c4cc(OC(F)F)ncc4OCC(C)(C)C#N)ccn3n2)n1]

The title compound was prepared using methods analogous to those described in Example 682. MS (ESI): mass calcd. for C₂₄H₂₄F₂N₈O₂, 494.2. m/z found, 495.2 [M+H]⁺. ¹H NMR (400 MHz, DMSO-d₆) δ 11.36 (s, 1H), 8.68 (d, J=7.3 Hz, 1H), 8.15 (s, 1H), 8.08 (s, 1H), 7.67 (t, J=73.1 Hz, 1H), 7.31 (s, 1H), 7.17 (d, J=7.3 Hz, 1H), 6.52 (s, 1H), 6.32 (s, 1H), 4.22 (s, 2H), 2.99 (d, J==4.4 Hz, 3H), 2.30 (s, 3H), 1.35 (s, 6H).

### Example 677: (1r,4r)-4-[[6-(difluoromethoxy)-4-[2-[(2,6-dimethylpyrimidin-4-yl)amino]pyrazolo[1,5-a]pyridin-5-yl]-3-pyridyl]oxy]cyclohexanol

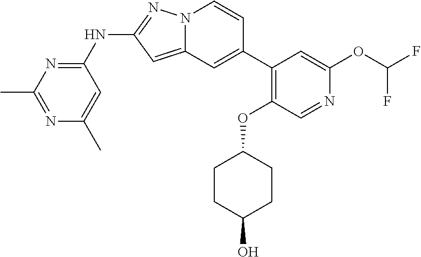 [SMILES: Cc1cc(Nc2cc3cc(-c4cc(OC(F)F)ncc4O[C@H]4CC[C@H](O)CC4)ccn3n2)nc(C)n1]

Step A: 5-(((1r,4r)-4-((tert-butyldimethylsilyl)oxy)cyclohexyl)oxy)-4-(2-((2,6-dimethylpyrimidin-4-yl)amino)pyrazolo[1,5-a]pyridin-5-yl)pyridin-2-ol. A mixture consisting of 5-(5-(((trans)-4-((tert-butyldimethylsilyl)oxy)cyclohexyl)oxy)-2-chloropyridin-4-yl)-N-(2,6-dimethylpyrimidin-4-yl)pyrazolo[1,5-a]pyridin-2-amine (Example 664, Step A, 1.0 g, 1.73 mmol), KOH (484 mg, 8.63 mmol), t-BuXphos (73.3 mg, 0.173 mmol), Pd₂(dba)₃ (158 mg, 0.173) and 1,4-dioxane/H₂O (30 mL, 1:1) was stirred at 106° C. overnight. The mixture was concentrated under reduced pressure to give a crude product, which was purified by FCC (eluent: petroleum ether/(ethyl acetate:EtOH=3:1) from 1:0 to 1:1) to afford the title compound (180 mg, 16%) as a yellow solid.

Step B: 5-(5-(((1r,4r)-4-((tert-butyldimethylsilyl)oxy)cyclohexyl)oxy)-2-(difluoromethoxy)pyridin-4-yl)-N-(2,6-dimethylpyrimidin-4-yl)pyrazolo[1,5-a]pyridin-2-amine. The title compound was prepared using methods analogous to those described in Example 329, Step C, and using 5-(((1r,4r)-4-((tert-butyldimethylsilyl)oxy)cyclohexyl)oxy)-4-(2-((2,6-dimethylpyrimidin-4-yl)amino)pyrazolo[1,5-a]pyridin-5-yl)pyridin-2-ol instead of 3-((6-hydroxy-4-iodopyridin-3-yl)oxy)-2,2-dimethylpropanenitrile.

Step C: 4-[[6-(difluoromethoxy)-4-[2-[(2,6-dimethylpyrimidin-4-yl)amino]pyrazolo[1,5-a]pyridin-5-yl]-3-pyridyl]oxy]cyclohexanol. The title compound was prepared using a TBDMS deprotection method similar to that described in Example 664, Step C, and using 5-(5-(((1r,4r)-4-((tert-butyldimethylsilyl)oxy)cyclohexyl)oxy)-2-(difluoromethoxy)pyridin-4-yl)-N-(2,6-dimethylpyrimidin-4-yl)pyrazolo[1,5-a]pyridin-2-amine instead of 5-(5-(((1r,4r)-4-((tert-butyldimethylsilyl)oxy)cyclohexyl)oxy)-2-(2,2,2-trifluoroethoxy)pyridin-4-yl)-N-(2,6-dimethylpyrimidin-4-yl)pyrazolo[1,5-a]pyridin-2-amine. MS (ESI): mass calcd. for C₂₅H₂₆F₂N₆O₃, 496.1. m/z found, 497.1 [M+H]⁺. ¹H NMR (400 MHz, DMSO-d₆) δ 10.24 (s, 1H), 8.60 (d, J=7.2 Hz, 1H), 8.17 (s, 1H), 7.84 (d, J=1.2 Hz, 1H), 7.65 (t, J=59.2 Hz, 1H), 7.22 (s, 1H), 7.05-6.97 (m, 2H), 6.89 (br. s., 1H), 4.55 (br. s., 1H), 4.47-4.35 (m, 1H), 3.56-3.44 (m, 1H), 2.46 (s, 3H), 2.30 (s, 3H), 2.02-1.95 (m, 2H), 1.76-1.69 (m, 2H), 1.46-1.38 (m, 2H), 1.33-1.25 (m, 2H). ¹⁹F NMR (376 MHz, DMSO-d₆) δ −86.26 (d, J=73.1 Hz, 2F)

### Example 678: 3-[[4-[2-[(2,6-dimethylpyrimidin-4-yl)amino]pyrazolo[1,5-a]pyridin-5-yl]-6-(2-hydroxyethoxy)-3-pyridyl]oxy]-2,2-dimethyl-propanenitrile

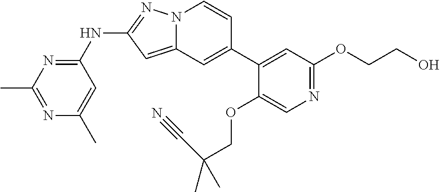 [SMILES: Cc1cc(Nc2cc3cc(-c4cc(OCCO)ncc4OCC(C)(C)C#N)ccn3n2)nc(C)n1]

Step A: 3-((6-(2-((tert-butyldimethylsilyl)oxy)ethoxy)-4-(2-((2,6-dimethylpyrimidin-4-yl)amino)pyrazolo[1,5-a]pyridin-5-yl)pyridin-3-yl)oxy)-2,2-dimethylpropanenitrile. The title compound was prepared using the coupling method described in Example 501 and using 34(6-chloro-4-(2-((2,6-dimethylpyrimidin-4-yl)amino)pyrazolo[1,5-a]pyridin-5-yl)pyridin-3-yl)oxy)-2,2-dimethylpropanenitrile and 2-((tert-butyldimethylsilyl)oxy)ethan-1-ol instead of tert-butyl (S)-2-(((6-chloro-4-(2-((2,6-dimethylpyrimidin-4-yl)amino)pyrazolo[1,5-a]pyridin-5-yl)pyridin-3-yl)oxy)methyl)morpholine-4-carboxylate and methylamine.

Step B: 3-[[4-[2-[(2,6-dimethylpyrimidin-4-yl)amino]pyrazolo[1,5-a]pyridin-5-yl]-6-(2-hydroxyethoxy)-3-pyridyl]oxy]-2,2-dimethyl-propanenitrile. The title compound was prepared using a TBDMS deprotection method similar to that described in Example 664, Step C, and using 3-((6-(2-((tert-butyldimethylsilyl)oxy)ethoxy)-4-(2-((2,6-dimethylpyrimidin-4-yl)amino)pyrazolo[1,5-a]pyridin-5-yl)pyridin-3-yl)oxy)-2,2-dimethylpropanenitrile instead of 5-(5-(((1r,4r)-4-((tert-butyldimethylsilyl)oxy)cyclohexyl)oxy)-2-(2,2,2-trifluoroethoxy)pyridin-4-yl)-N-(2,6-dimethylpyrimidin-4-yl)pyrazolo[1,5-a]pyridin-2-amine. MS (ESI): mass calcd. for C₂₅H₂₇N₇O₃, 473.1. m/z found, 474.1 [M+H]⁺. ¹H NMR (400 MHz, DMSO-d₆) δ 10.23 (s, 1H), 8.59 (d, J=7.2 Hz, 1H), 8.02 (s, 1H), 7.87 (d, J=1.2 Hz, 1H), 7.09-7.00 (m, 2H), 6.95 (s, 1H), 6.85 (s, 1H), 4.85 (t, J=5.6 Hz, 1H), 4.27 (t, J=4.8 Hz, 2H), 4.11 (s, 2H), 3.78-3.66 (m, 2H), 2.45 (s, 3H), 2.30 (s, 3H), 1.34 (s, 6H).

### Example 679: 1-[[6-(difluoromethoxy)-4-[2-[(2,6-dimethylpyrimidin-4-yl)amino]pyrazolo[1,5-a]pyridin-5-yl]-3-pyridyl]oxy]-2-methyl-propan-2-ol

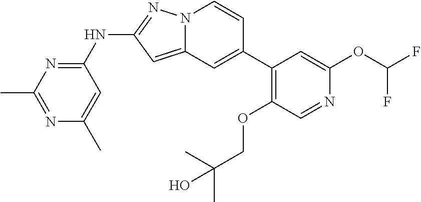 [SMILES: Cc1cc(Nc2cc3cc(-c4cc(OC(F)F)ncc4OCC(C)(C)O)ccn3n2)nc(C)n1]

To a mixture consisting of 6-(difluoromethoxy)-4-(2-((2,6-dimethylpyrimidin-4-yl)amino)pyrazolo[1,5-a]pyridin-5-yl)pyridin-3-ol (120 mg, 0.301 mmol), 2-hydroxy-2-methylpropyl 4-methylbenzenesulfonate2-hydroxy-2-methylpropyl 4-methylbenzenesulfonate (81 mg, 0.33 mmol), KI (50 mg, 0.30 mmol) and DMF (2 mL) was added Cs₂CO₃ (245 mg, 0.753 mmol), and the resultant solution was stirred at 110° C. for 3 h. The reaction mixture was continued to heated to 120° C. and stirred for 3 h. H₂O (10 mL) was added to the reaction mixture, and the mixture was extracted with EtOAc (30 mL×2), combined extracts and concentrated under vacuum to give a residue, which was purified by preparative HPLC DB using a Boston Prime C18 150×30 mm×5 um column ((eluent: 40% to 70% (v/v) water (0.05% NH₃H₂O+10 mM NH₄HCO₃)-ACN to afford pure product, which was suspended in water (20 mL), the mixture frozen using dry ice/ethanol, and then lyophilized to dryness to afford the title compound (24.30 mg, 17%) as a white solid. MS (ESI): mass calcd. for C₂₃H₂₄F₂N₆O₃, 470.1. m/z found, 471.1 [M+H]⁺. ¹H NMR (400 MHz, CDCl₃) δ 8.40 (d, J=7.2 Hz, 1H), 7.91 (s, 1H), 7.61 (d, J=0.8 Hz, 1H), 7.5 (t, J=73.2, 1H), 7.48 (s, 1H), 7.06 (s, 1H), 6.96 (s, 1H), 6.90 (dd, J=2.0, 7.2 Hz, 1H), 6.61 (s, 1H), 3.89 (s, 2H), 2.60 (s, 3H), 2.45 (s, 3H), 1.99 (br. s., 1H), 1.29 (s, 6H). ¹⁹F NMR (376 MHz, CDCl₃) δ −88.19 (s, 2F)

### Example 680: (*R)-3-[[6-(1-cyanoethoxy)-4-[2-[(2,6-dimethylpyrimidin-4-yl)amino]pyrazolo[1,5-a]pyridin-5-yl]-3-pyridyl]oxy]-2,2-dimethyl-propanenitrile

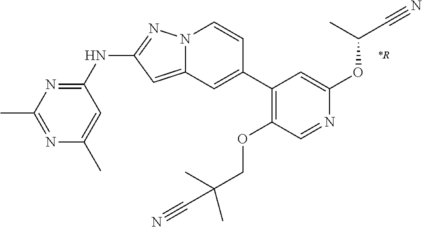 [SMILES: Cc1cc(Nc2cc3cc(-c4cc(O[C@H](C)C#N)ncc4OCC(C)(C)C#N)ccn3n2)nc(C)n1]

Step A: 3-((4-(2-((2,6-dimethylpyrimidin-4-yl)amino)pyrazolo[1,5-a]pyridin-5-yl)-6-hydroxypyridin-3-yl)oxy)-2,2-dimethylpropanenitrile. A mixture consisting of 3-((6-chloro-4-(2-((2,6-dimethylpyrimidin-4-yl)amino)pyrazolo[1,5-a]pyridin-5-yl)pyridin-3-yl)oxy)-2,2-dimethylpropanenitrile (Example 667, Step A, 3.0 g, 6.7 mmol), KOH (1.5 g, 27 mmol), Pd₂(dba)₃ (613 mg, 0.670 mmol), t-BuXphos (284 mg, 0.670 mmol), 1,4-dioxane (30 mL) and H₂O (5.4 mL) was purged with N₂ for 1 min and the mixture was stirred at 120° C. overnight. The reaction mixture was concentrated to dryness under reduced pressure to give the crude product, which was purified by FCC (eluent: MeOH in CH₂Cl₂ from 0 to 50%) to afford the title product (320 mg, 9%) as a yellow solid.

Step B: 3-((6-(1-cyanoethoxy)-4-(2-((2,6-dimethylpyrimidin-4-yl)amino)pyrazolo[1,5-a]pyridin-5-yl)pyridin-3-yl)oxy)-2,2-dimethylpropanenitrile. A mixture consisting of 3-((4-(2-((2,6-dimethylpyrimidin-4-yl)amino)pyrazolo[1,5-a]pyridin-5-yl)-6-hydroxypyridin-3-yl)oxy)-2,2-dimethylpropanenitrile (200 mg, 0.391 mmol), 2-bromopropanenitrile (52.4 mg, 0.391 mmol), Ag₂CO₃ (324 mg, 1.17 mmol), KI (97.4 mg, 0.587 mmol) and toluene (6 mL) was stirred at 120° C. for 12 h. The reaction mixture was concentrated to dryness under reduced pressure to give the crude product, which was purified by FCC (eluent: MeOH in CH₂Cl₂ from 0 to 50%) to afford the title product. The crude product was further purified by preparative HPLC using a Boston Prime C18 150×30 mm×5 μm (eluent: 50% to 80% (v/v) water (0.05% NH₃H₂O+10 mM NH₄HCO₃)-ACN to afford pure product. The product was suspended in water (10 mL), the mixture frozen using dry ice/ethanol, and then lyophilized to dryness to afford the title compound (30 mg, 16%) as a white solid.

Step C: (*R)-3-[[6-(1-cyanoethoxy)-4-[2-[(2,6-dimethylpyrimidin-4-yl)amino]pyrazolo[1,5-a]pyridin-5-yl]-3-pyridyl]oxy]-2,2-dimethyl-propanenitrile. 3-((6-(1-cyanoethoxy)-4-(2-((2,6-dimethylpyrimidin-4-yl)amino)pyrazolo[1,5-a]pyridin-5-yl)pyridin-3-yl)oxy)-2,2-dimethylpropanenitrile (30 mg, 0.062 mmol) was purified by SFC-22 over DAICEL CHIRALPAK AD 250 mm×30 mm, 10 μm (eluent: 40% to 40% (v/v) supercritical water (Neu-IPA). The pure fractions were collected and the volatiles were removed under reduced pressure. The product was suspended in water (10 mL), the mixture frozen using dry ice/ethanol, and then lyophilized to dryness to afford the title compound (3.5 mg, 12%) as a white solid. MS (ESI): mass calcd. for C₂₆H₂₆N₈O₂, 482.1. m/z found, 483.1 [M+H]⁺. ¹H NMR (400 MHz, DMSO-d₆) δ 10.23 (s, 1H), 8.60 (d, J=7.2 Hz, 1H), 8.12 (s, 1H), 7.90 (d, J=1.2 Hz, 1H), 7.12 (s, 1H), 7.07-7.01 (m, 2H), 6.86 (br. s., 1H), 5.72 (q, J=6.8 Hz, 1H), 4.18 (s, 2H), 2.45 (s, 3H), 2.30 (s, 3H), 1.71 (d, J=6.8 Hz, 3H), 1.35 (s, 6H).

### Example 681: (*S)-3-[[6-(1-cyanoethoxy)-4-[2-[(2,6-dimethylpyrimidin-4-yl)amino]pyrazolo[1,5-a]pyridin-5-yl]-3-pyridyl]oxy]-2,2-dimethyl-propanenitrile

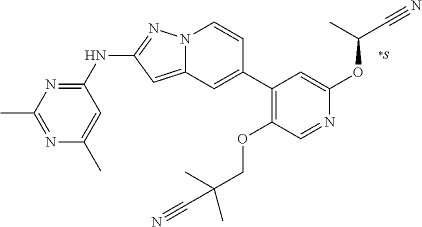 [SMILES: Cc1cc(Nc2cc3cc(-c4cc(O[C@@H](C)C#N)ncc4OCC(C)(C)C#N)ccn3n2)nc(C)n1]

The title compound (3.0 mg, 10%) was the other peak isolated from the SFC purification in Example 680, Step C. MS (ESI): mass calcd. for C₂₆H₂₆N₈O₂, 482.2. m/z found, 483.2 [M+H]⁺. ¹H NMR (400 MHz, DMSO-d₆) δ 10.23 (s, 1H), 8.60 (d, J=7.2 Hz, 1H), 8.12 (s, 1H), 7.90 (d, J=1.2 Hz, 1H), 7.12 (s, 1H), 7.08-7.00 (m, 2H), 6.86 (br. s., 1H), 5.72 (q, J=6.8 Hz, 1H), 4.18 (s, 2H), 2.45 (s, 3H), 2.30 (s, 3H), 1.71 (d, J=6.8 Hz, 3H), 1.35 (s, 6H).

### Example 682: 3-[[4-[2-[(2-amino-6-methyl-pyrimidin-4-yl)amino]pyrazolo[1,5-a]pyridin-5-yl]-6-(difluoromethoxy)-3-pyridyl]oxy]-2,2-dimethyl-propanenitrile

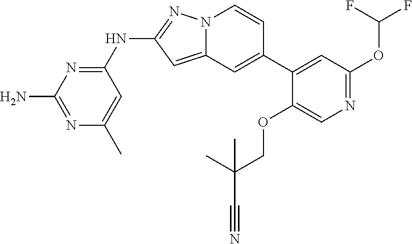 [SMILES: Cc1cc(Nc2cc3cc(-c4cc(OC(F)F)ncc4OCC(C)(C)C#N)ccn3n2)nc(N)n1]

To a solution of Intermediate 27 (75 mg, 0.20 mmol, 1.00 equiv), 2-amino-4-chloro-6-methylpyrimidine (33 mg, 0.22 mmol, 1.1 equiv), cesium carbonate (135 mg, 0.42 mmol, 2.1 equiv), Xantphos Pd G3 (5 mol %) in 1,4-dioxane (3.3 mL) was purged with N₂ (evacuate and back-filled with N₂×3) and the resulting mixture stirred at 90° C. for 16 hour. The crude mixture was then filtered through celite and concentrated in vacuo. The crude oil was purified by reverse phase HPLC (MeCN/H₂O with 20 mM NH₄OH) to give the title compound (28 mg, 28% yield). MS (ESI): mass calcd. for C₂₃H₂₂F₂N802, 480.2. m/z found, 481.2 [M+H]⁺. ¹H NMR (500 MHz, DMSO-d₆) δ 12.69 (s, 1H), 11.24 (s, 1H), 8.68 (dt, J=7.3, 0.9 Hz, 1H), 8.16 (s, 1H), 7.84 (s, 1H), 7.68 (t, J=73.1 Hz, 1H), 7.28 (s, 1H), 7.14 (dd, J=7.2, 2.0 Hz, 1H), 6.34 (s, 1H), 4.21 (s, 2H), 2.30 (s, 3H), 1.35 (s, 6H).

### Example 683: 3-[[6-(difluoromethoxy)-4-[2-[(6-methylpyrazin-2-yl)amino]pyrazolo[1,5-a]pyridin-5-yl]-3-pyridyl]oxy]-2,2-dimethyl-propanenitrile

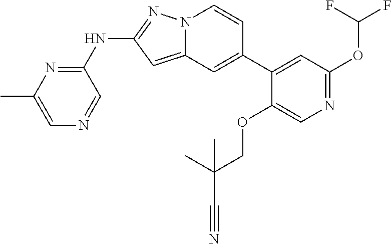 [SMILES: Cc1cncc(Nc2cc3cc(-c4cc(OC(F)F)ncc4OCC(C)(C)C#N)ccn3n2)n1]

The title compound was prepared using a similar method described in Example 682. MS (ESI): mass calcd. for C₂₃H₂₁F₂N₇O₂, 465.2. m/z found, 466.2 [M+H]⁺. ¹H NMR (500 MHz, DMSO-d₆) δ 10.19 (s, 1H), 8.61 (d, J=7.1 Hz, 1H), 8.50 (s, 1H), 8.14 (s, 1H), 7.92 (q, J=1.0 Hz, 2H), 7.67 (t, J=73.2 Hz, 1H), 7.27 (s, 1H), 7.05 (dd, J=7.2, 2.0 Hz, 1H), 6.91 (d, J=0.8 Hz, 1H), 4.21 (s, 2H), 2.41 (s, 3H), 1.36 (s, 6H).

### Example 684: 3-[[6-(difluoromethoxy)-4-[2-[(1,5-dimethyl-6-oxo-pyridazin-3-yl)amino]pyrazolo[1,5-a]pyridin-5-yl]-3-pyridyl]oxy]-2,2-dimethyl-propanenitrile

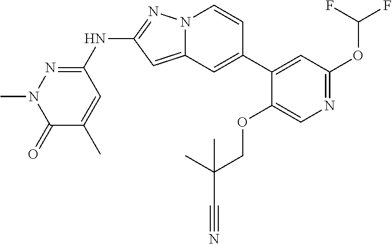 [SMILES: Cc1cc(Nc2cc3cc(-c4cc(OC(F)F)ncc4OCC(C)(C)C#N)ccn3n2)nn(C)c1=O]

The title compound was prepared using a similar method described in Example 682, except XantPhos Pd G3 was used as the catalyst. MS (ESI): mass calcd. for C₂₄H₂₃F₂N₇O₃, 495.2. m/z found, 496.2 [M+H]⁺. ¹H NMR (500 MHz, DMSO-d₆) δ 9.78 (s, 1H), 8.56 (d, J=7.2 Hz, 1H), 8.13 (s, 1H), 7.91 (dd, J=2.0, 0.9 Hz, 1H), 7.83-7.50 (m, 1H), 7.27 (s, 1H), 7.24 (d, J=1.4 Hz, 1H), 7.06 (dd, J=7.2, 2.0 Hz, 1H), 6.87 (d, J=0.9 Hz, 1H), 4.20 (s, 2H), 3.60 (s, 3H), 2.09 (d, J=1.3 Hz, 3H), 1.35 (s, 6H).

### Example 685: 3-[[6-(difluoromethoxy)-4-[2-[(6-methylpyrimidin-4-yl)amino]pyrazolo[1,5-a]pyridin-5-yl]-3-pyridyl]oxy]-2,2-dimethyl-propanenitrile

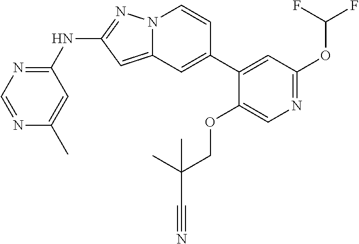 [SMILES: Cc1cc(Nc2cc3cc(-c4cc(OC(F)F)ncc4OCC(C)(C)C#N)ccn3n2)ncn1]

The title compound was prepared using a similar method described in Example 682, except RuPhos Pd G3 was used as the catalyst. MS (ESI): mass calcd. for C₂₃H₂₁F₂N₇O₂, 465.1. m/z found, 466.1 [M+H]⁺. ¹H NMR (500 MHz, CDCl₃) δ 8.69 (d, J=1.1 Hz, 1H), 8.41 (dd, J=7.3, 0.9 Hz, 1H), 7.87 (s, 1H), 7.72 (dd, J=2.0, 0.9 Hz, 1H), 7.56-7.27 (m, 2H), 7.01-6.97 (m, 2H), 6.64 (s, 1H), 3.97 (s, 2H), 2.49 (s, 3H), 1.41 (s, 6H).

### Example 686: 3-[[4-[2-[(2,6-dimethylpyrimidin-4-yl)amino]pyrazolo[1,5-a]pyridin-5-yl]-6-methyl-3-pyridyl]oxymethyl]cyclobutanecarbonitrile

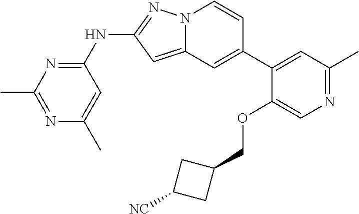

The title compound was prepared using the method described in Example 198 and using 3-(hydroxymethyl)cyclobutane-1-carbonitrile instead of trans-1,4-cyclohexanediol. A subsequent SFC purification step, similar to that described in Example 5, yielded the title compound. MS (ESI): mass calcd. for C₂₅H₂₅N₇O, 439.2. m/z found, 440.1 [M+H]⁺. ¹H NMR (400 MHz, CDCl₃) δ 8.39 (d, J=7.2 Hz, 1H), 8.25 (s, 1H), 7.59 (d, J=1.2 Hz, 1H), 7.52 (s, 1H), 7.18 (s, 1H), 7.09 (s, 1H), 6.90 (dd, J=2.0, 7.6 Hz, 1H), 6.59 (s, 1H), 4.06 (d, J=5.2 Hz, 2H), 3.09-2.94 (m, 2H), 2.61 (s, 3H), 2.57 (s, 3H), 2.54-2.47 (m, 2H), 2.46 (s, 3H), 2.38-2.29 (m, 2H).

### Example 687: 3-[[4-[2-[(2,6-dimethylpyrimidin-4-yl)amino]pyrazolo[1,5-a]pyridin-5-yl]-6-methyl-3-pyridyl]oxymethyl]cyclobutanecarbonitrile

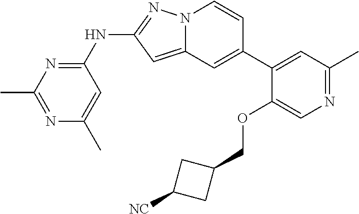

The title compound was the other isolated fraction from the SFC purification step in Example 686. MS (ESI): mass calcd. for C₂₅H₂₅N₇O, 439.2. m/z found, 440.1 [M+H]⁺. ¹H NMR (400 MHz, CDCl₃) δ 8.43 (d, J=7.2 Hz, 1H), 8.24 (s, 1H), 7.62 (d, J=1.2 Hz, 1H), 7.52 (s, 1H), 7.20 (s, 1H), 7.10 (s, 1H), 6.96 (dd, J=2.0, 7.2 Hz, 1H), 6.62 (s, 1H), 4.03 (d, J=5.2 Hz, 2H), 3.08-2.95 (m, 1H), 2.86-2.71 (m, 1H), 2.60 (s, 3H), 2.57 (s, 3H), 2.50-2.41 (m, 5H), 2.40-2.30 (m, 2H).

### Example 688: 3-[[6-(2,2-difluoroethoxy)-4-[2-(pyridazin-3-ylamino)pyrazolo[1,5-a]pyridin-5-yl]-3-pyridyl]oxy]-2,2-dimethyl-propanenitrile

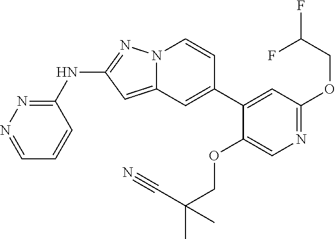 [SMILES: CC(C)(C#N)COc1cnc(OCC(F)F)cc1-c1ccn2nc(Nc3cccnn3)cc2c1]

To a solution of Intermediate 28 (69 mg, 0.18 mmol, 1.00 equiv), 3-bromopyridazine (32 mg, 0.20 mmol, 1.1 equiv), cesium carbonate (180 mg, 0.55 mmol, 3.1 equiv), Xantphos Pd G3 (10 mol %) in 1,4-dioxane (1.5 mL) was purged with N₂ (evacuate and back-filled with N₂×3) and the resulting mixture stirred at 90° C. for 16 hour. The crude mixture was then filtered through celite and concentrated in vacuo. The crude oil was purified by reverse phase HPLC (MeCN/H₂O with 20 mM NH₄OH) to give the title compound (8 mg, 0.017 mmol, 10% yield). MS (ESI): mass calcd. for C₂₃H₂₁F₂N₇O₂, 465.2. m/z found, 466.1 [M+H]⁺. ¹H NMR (400 MHz, CDCl₃) δ 8.70 (dd, J=4.6, 1.4 Hz, 1H), 8.32 (dt, J=7.2, 0.9 Hz, 1H), 8.07 (s, 1H), 7.95 (dd, J=9.1, 1.4 Hz, 1H), 7.74 (s, 1H), 7.60 (dd, J=2.0, 0.9 Hz, 1H), 7.33 (dd, J=9.1, 4.6 Hz, 1H), 6.89 (dd, J=7.2, 2.0 Hz, 1H), 6.84 (d, J=0.5 Hz, 1H), 6.51 (d, J=0.8 Hz, 1H), 6.06 (tt, J=55.6, 4.2 Hz, 1H), 4.46 (td, J=13.5, 4.2 Hz, 2H), 3.84 (s, 2H), 1.32 (s, 6H).

### Example 689: 3-[[6-(2,2-difluoroethoxy)-4-[2-(pyrazin-2-ylamino)pyrazolo[1,5-a]pyridin-5-yl]-3-pyridyl]oxy]-2,2-dimethyl-propanenitrile

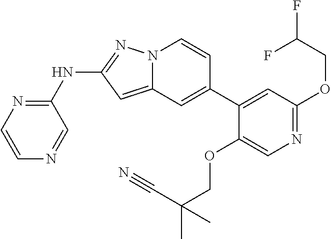 [SMILES: CC(C)(C#N)COc1cnc(OCC(F)F)cc1-c1ccn2nc(Nc3cnccn3)cc2c1]

The title compound was prepared using a similar method described in Example 688. MS (ESI): mass calcd. for C₂₃H₂₁F₂N₇O₂, 465.2. m/z found, 466.1 [M+H]⁺. ¹H NMR (400 MHz, CDCl₃) δ 8.66 (d, J=1.5 Hz, 1H), 8.32 (dd, J=7.3, 1.1 Hz, 1H), 8.13 (dd, J=2.8, 1.5 Hz, 1H), 8.01 (d, J==2.6 Hz, 1H), 7.74 (s, 1H), 7.61 (d, J=2.2 Hz, 2H), 6.89 (dd, J=7.1, 2.0 Hz, 1H), 6.83 (s, 1H), 6.66 (s, 1H), 6.06 (tt, J=55.6, 4.2 Hz, 1H), 4.46 (td, J=13.6, 4.2 Hz, 2H), 3.84 (s, 2H), 1.32 (s, 6H).

### Example 690: 2-[[4-[2-[(2,6-dimethylpyrimidin-4-yl)amino]pyrazolo[1,5-a]pyridin-5-yl]-5-(4-hydroxycyclohexoxy)-2-pyridyl]oxy]acetonitrile

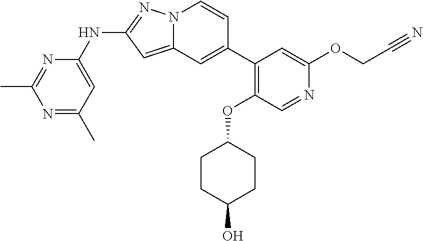 [SMILES: Cc1cc(Nc2cc3cc(-c4cc(OCC#N)ncc4O[C@H]4CC[C@H](O)CC4)ccn3n2)nc(C)n1]

Step A: 5-(2-chloro-5-(((1r,4r)-4-((tetrahydro-2H-pyran-2-yl)oxy)cyclohexyl)oxy)pyridin-4-yl)-N-(2,6-dimethylpyrimidin-4-yl)pyrazolo[1,5-a]pyridin-2-amine. The title compound was prepared using the method described in Example 664, Step A, and using (trans)-4-((tetrahydro-2H-pyran-2-yl)oxy)cyclohexanol instead of (1r,4r)-4-((tert-butyldimethylsilyl)oxy)cyclohexanol.

Step B: 4-(2-((2,6-dimethylpyrimidin-4-yl)amino)pyrazolo[1,5-a]pyridin-5-yl)-5-(((1r,4r)-4-((tetrahydro-2H-pyran-2-yl)oxy)cyclohexyl)oxy)pyridin-2-ol. The title compound was prepared using the method described in Example 677, Step A, and using 5-(2-chloro-5-(((1r,4r)-4-((tetrahydro-2H-pyran-2-yl)oxy)cyclohexyl)oxy)pyridin-4-yl)-N-(2,6-dimethylpyrimidin-4-yl)pyrazolo[1,5-a]pyridin-2-amine instead of 5-(5-(((trans)-4-((tert-butyldimethylsilyl)oxy)cyclohexyl)oxy)-2-chloropyridin-4-yl)-N-(2,6-dimethylpyrimidin-4-yl)pyrazolo[1,5-a]pyridin-2-amine.

Step C: 4-(2-((2,6-dimethylpyrimidin-4-yl)amino)pyrazolo[1,5-a]pyridin-5-yl)-5-(((1r,4r)-4-hydroxycyclohexyl)oxy)pyridin-2-ol. A solution of 4-(2-((2,6-dimethylpyrimidin-4-yl)amino)pyrazolo[1,5-a]pyridin-5-yl)-5-(((1r,4r)-4-((tetrahydro-2H-pyran-2-yl)oxy)cyclohexyl)oxy)pyridin-2-ol (470 mg, 0.886 mmol) and HCl/1,4-dioxane (10 mL, 4 M) was stirred at r.t. for 1 h. The mixture was concentrated under reduced pressure. The mixture was neutralized with aq. NaHCO₃ and concentrated under reduced pressure. Then the mixture was slurried in (10 mL MeCN and 2 mL MeOH) and collected by filtration and afford the crude compound (400 mg, crude) as a yellow solid.

Step D: 2-[[4-[2-[(2,6-dimethylpyrimidin-4-yl)amino]pyrazolo[1,5-a]pyridin-5-yl]-5-(4-hydroxycyclohexoxy)-2-pyridyl]oxy]acetonitrile. The title compound was prepared using methods analogous to those described in Example 680, Step B. MS (ESI): mass calcd. for C₂₆H₂₇N₇O₃, 485.2. m/z found, 486.1 [M+H]⁺. ¹H NMR (400 MHz, CDCl₃) δ 8.37 (d, J=7.2 Hz, 1H), 7.94 (s, 1H), 7.63 (s, 1H), 7.43 (s, 1H), 7.07 (s, 1H), 6.96-6.91 (m, 1H), 6.89 (s, 1H), 6.61 (s, 1H), 5.02 (s, 2H), 4.25-4.11 (m, 1H), 3.81-3.68 (m, 1H), 2.61 (s, 3H), 2.46 (s, 3H), 2.12-2.00 (m, 2H), 1.96-1.86 (m, 2H), 1.53-1.46 (m, 2H), 1.44-1.32 (m, 2H).

### Example 691: 3-[[4-[2-[(6-aminopyrazin-2-yl)amino]pyrazolo[1,5-a]pyridin-5-yl]-6-(difluoromethoxy)-3-pyridyl]oxy]-2,2-dimethyl-propanenitrile

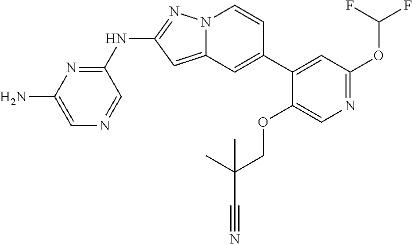 [SMILES: CC(C)(C#N)COc1cnc(OC(F)F)cc1-c1ccn2nc(Nc3cncc(N)n3)cc2c1]

The title compound was prepared using a similar method described in Example 682, except tBuXPhos Pd G3 was used as the catalyst. MS (ESI): mass calcd. for C₂₂H₂₀F₂N₈O₂, 466.2. m/z found, 467.1 [M+H]⁺. ¹H NMR (500 MHz, CDCl₃) δ 8.39 (dt, J=7.1, 0.9 Hz, 1H), 8.03 (s, 1H), 7.86 (s, 1H), 7.75 (s, 1H), 7.67 (dd, J=2.0, 0.9 Hz, 1H), 7.55-7.25 (m, 2H), 6.99 (s, 1H), 6.94 (dd, J=7.2, 2.0 Hz, 1H), 6.70 (d, J=0.8 Hz, 1H), 4.68 (s, 2H), 3.96 (s, 2H), 1.40 (s, 6H).

### Example 692: 3-[[6-(difluoromethoxy)-4-[2-[(1-methylpyrazol-4-yl)amino]pyrazolo[1,5-a]pyridin-5-yl]-3-pyridyl]oxy]-2,2-dimethyl-propanenitrile

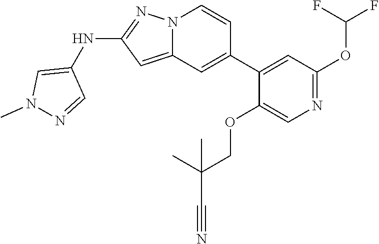 [SMILES: Cn1cc(Nc2cc3cc(-c4cc(OC(F)F)ncc4OCC(C)(C)C#N)ccn3n2)cn1]

The title compound was prepared using a similar method described in Example 682, except tBuXPhos Pd G3 was used as the catalyst. MS (ESI): mass calcd. for C₂₂H₂₁F₂N₇O₂, 453.2. m/z found, 454.1 [M+H]⁺. ¹H NMR (500 MHz, CDCl₃) δ 8.28 (dt, J=7.2, 0.9 Hz, 1H), 7.85 (s, 1H), 7.61 (s, 1H), 7.55-7.24 (m, 3H), 6.97 (s, 1H), 6.80 (dd, J=7.2, 2.0 Hz, 1H), 5.91 (d, J=0.8 Hz, 1H), 5.82 (s, 1H), 3.94 (s, 2H), 3.90 (s, 3H), 1.39 (s, 6H).

### Example 693: 3-[[6-(2,2-difluoroethoxy)-4-[2-[(1,5-dimethyl-6-oxo-pyridazin-3-yl)amino]pyrazolo[1,5-a]pyridin-5-yl]-3-pyridyl]oxy]-2,2-dimethyl-propanenitrile

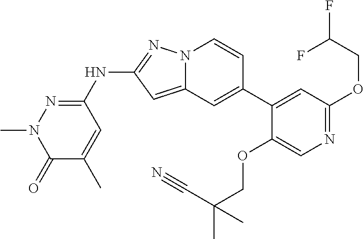 [SMILES: Cc1cc(Nc2cc3cc(-c4cc(OCC(F)F)ncc4OCC(C)(C)C#N)ccn3n2)nn(C)c1=O]

The title compound was prepared using a similar method described in Example 688. MS (ESI): mass calcd. for C₂₅H₂₅F₂N₇O₃, 509.2. m/z found, 510.1 [M+H]⁺. ¹H NMR (400 MHz, CDCl₃) δ 8.34 (dt, J=7.2, 1.0 Hz, 1H), 7.85 (s, 1H), 7.73 (dd, J=2.0, 0.9 Hz, 1H), 7.21-7.16 (m, 1H), 7.13 (d, J=1.3 Hz, 1H), 7.02 (dd, J=7.2, 2.0 Hz, 1H), 6.93 (s, 1H), 6.76 (d, J=0.8 Hz, 1H), 6.16 (tt, J=55.6, 4.2 Hz, 1H), 4.56 (td, J=13.6, 4.2 Hz, 2H), 3.94 (s, 2H), 3.79 (s, 3H), 2.28 (d, J=1.2 Hz, 3H), 1.42 (s, 6H).

### Example 694: 3-[[6-(difluoromethoxy)-4-[2-(3-pyridylamino)pyrazolo[1,5-a]pyridin-5-yl]-3-pyridyl]oxy]-2,2-dimethyl-propanenitrile

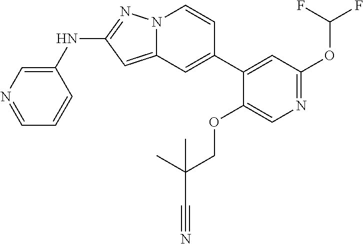 [SMILES: CC(C)(C#N)COc1cnc(OC(F)F)cc1-c1ccn2nc(Nc3cccnc3)cc2c1]

The title compound was prepared using a similar method described in Example 682, except tBuXPhos Pd G3 was used as the catalyst. MS (ESI): mass calcd. for C₂₃H₂₀F₂N₆O₂, 450.2. m/z found, 451.1 [M+H]⁺. ¹H NMR (500 MHz, CDCl₃) δ 8.59-8.57 (m, 1H), 8.35 (dt, J=7.1, 0.9 Hz, 1H), 8.19 (dd, J=4.6, 1.4 Hz, 1H), 7.93-7.87 (m, 1H), 7.85 (s, 1H), 7.64 (dd, J=2.0, 0.9 Hz, 1H), 7.55-7.26 (m, 1H), 7.26-7.23 (m, 1H), 6.98 (s, 1H), 6.89 (dd, J=7.2, 2.0 Hz, 1H), 6.72 (s, 1H), 6.19 (d, J=0.8 Hz, 1H), 3.95 (s, 2H), 1.40 (s, 6H).

### Example 695: 3-[[6-(difluoromethoxy)-4-[2-[[5-(morpholinomethyl)-2-pyridyl]amino]pyrazolo[1,5-a]pyridin-5-yl]-3-pyridyl]oxy]-2,2-dimethyl-propanenitrile

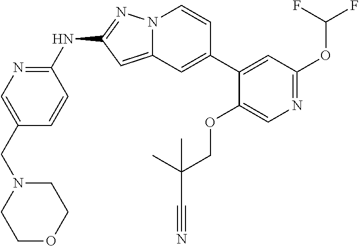

The title compound was prepared using a similar method described in Example 682, except tBuXPhos Pd G3 was used as the catalyst. MS (ESI): mass calcd. for C₂₈H₂₉F₂N₇O₃, 549.2. m/z found, 550.1 [M+H]⁺. ¹H NMR (500 MHz, CDCl₃) δ 8.42-8.36 (m, 1H), 8.20-8.18 (m, 1H), 7.86 (s, 1H), 7.83 (s, 1H), 7.65 (dd, J=2.0, 0.9 Hz, 1H), 7.61 (dd, J=8.5, 2.3 Hz, 1H), 7.55-7.25 (m, 2H), 6.99 (s, 1H), 6.91 (dd, J=7.1, 2.0 Hz, 1H), 6.62 (d, J=0.8 Hz, 1H), 3.95 (s, 2H), 3.70 (t, J=4.7 Hz, 4H), 3.44 (s, 2H), 2.46-2.44 (m, 4H), 1.39 (s, 6H).

### Example 696: 3-[[6-(2,2-difluoroethoxy)-4-[2-[(6-methylpyrazin-2-yl)amino]pyrazolo[1,5-a]pyridin-5-yl]-3-pyridyl]oxy]-2,2-dimethyl-propanenitrile

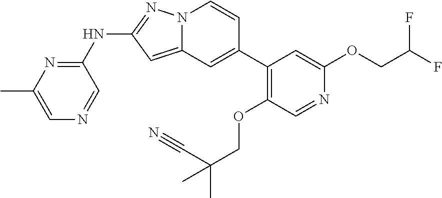 [SMILES: Cc1cncc(Nc2cc3cc(-c4cc(OCC(F)F)ncc4OCC(C)(C)C#N)ccn3n2)n1]

The title compound was prepared using a similar method described in Example 688. MS (ESI): mass calcd. for C₂₄H₂₃F₂N₇O₂, 479.2. m/z found, 480.1 [M+H]⁺. ¹H NMR (400 MHz, CDCl₃) δ 8.58 (s, 1H), 8.37 (dt, J=7.2, 0.9 Hz, 1H), 7.98 (s, 1H), 7.82 (s, 1H), 7.68 (dd, J=2.0, 0.9 Hz, 1H), 7.31 (s, 1H), 6.96 (dd, J=7.2, 2.0 Hz, 1H), 6.91 (s, 1H), 6.70 (d, J=0.8 Hz, 1H), 6.14 (tt, J=55.6, 4.2 Hz, 1H), 4.54 (td, J=13.5, 4.2 Hz, 2H), 3.91 (s, 2H), 2.49 (t, J=0.7 Hz, 3H), 1.39 (s, 6H).

### Example 697: 3-[[6-(2,2-difluoroethoxy)-4-[2-[(1-methyl-6-oxo-pyridazin-3-yl)amino]pyrazolo[1,5-a]pyridin-5-yl]-3-pyridyl]oxy]-2,2-dimethyl-propanenitrile

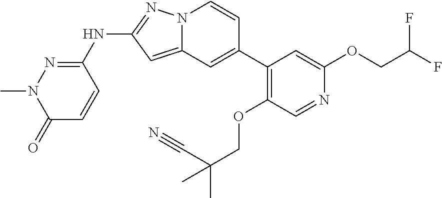 [SMILES: Cn1nc(Nc2cc3cc(-c4cc(OCC(F)F)ncc4OCC(C)(C)C#N)ccn3n2)ccc1=O]

The title compound was prepared using a similar method described in Example 688. MS (ESI): mass calcd. for C₂₄H₂₃F₂N₇O₃, 495.2. m/z found, 496.1 [M+H]⁺. ¹H NMR (400 MHz, CDCl₃) δ 8.23 (dt, J=7.2, 0.9 Hz, 1H), 7.74 (s, 1H), 7.62 (dd, J=2.0, 0.9 Hz, 1H), 7.24 (d, J=9.8 Hz, 1H), 6.91-6.81 (m, 4H), 6.60 (d, J=0.8 Hz, 1H), 6.06 (tt, J=55.6, 4.2 Hz, 1H), 4.46 (td, J=13.5, 4.1 Hz, 2H), 3.84 (s, 2H), 3.68 (s, 3H), 1.32 (s, 6H).

### Example 698: 3-[[6-(difluoromethoxy)-4-[2-[(4-methoxypyrimidin-2-yl)amino]pyrazolo[1,5-a]pyridin-5-yl]-3-pyridyl]oxy]-2,2-dimethyl-propanenitrile

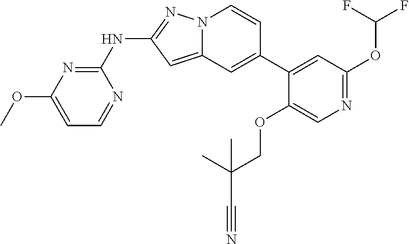 [SMILES: COc1ccnc(Nc2cc3cc(-c4cc(OC(F)F)ncc4OCC(C)(C)C#N)ccn3n2)n1]

The title compound was prepared using a similar method described in Example 682, except tBuXPhos Pd G3 was used as the catalyst. MS (ESI): mass calcd. for C₂₃H₂₁F₂N₇O₃, 481.2. m/z found, 482.1 [M+H]⁺. ¹H NMR (500 MHz, DMSO-d₆) δ 10.50 (s, 1H), 8.63-8.56 (m, 1H), 8.14 (s, 1H), 7.95 (dd, J=2.1, 1.0 Hz, 1H), 7.84-7.50 (m, 2H), 7.27 (s, 1H), 7.19 (s, 1H), 7.09 (dd, J==7.2, 2.0 Hz, 1H), 6.14 (s, 1H), 4.21 (s, 2H), 3.31 (s, 3H), 1.36 (s, 6H).

### Example 699: 3-[[6-(difluoromethoxy)-4-[2-[(1-methyl-1,2,4-triazol-3-yl)amino]pyrazolo[1,5-a]pyridin-5-yl]-3-pyridyl]oxy]-2,2-dimethyl-propanenitrile

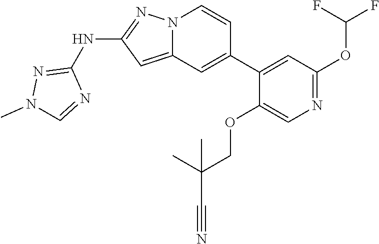 [SMILES: Cn1cnc(Nc2cc3cc(-c4cc(OC(F)F)ncc4OCC(C)(C)C#N)ccn3n2)n1]

The title compound was prepared using a similar method described in Example 682, except tBuXPhos Pd G3 was used as the catalyst. MS (ESI): mass calcd. for C₂₁H₂₀F₂N₈O₂, 454.2. m/z found, 455.1 [M+H]⁺. ¹H NMR (400 MHz, CDCl₃) CDCl 8.63-8.57 (m, 1H), 8.46 (d, J=3.1 Hz, 1H), 7.87 (s, 1H), 7.85-7.81 (m, 1H), 7.61 (dd, J=2.0, 0.9 Hz, 1H), 7.60-7.22 (m, 1H), 6.99 (s, 1H), 6.93 (dd, J=7.2, 2.0 Hz, 1H), 6.90 (d, J=0.8 Hz, 1H), 3.94 (s, 2H), 3.89 (d, J=0.6 Hz, 3H), 1.39 (s, 6H).

### Example 700: 3-[[6-(difluoromethoxy)-4-[2-[[2-(ethylamino)-6-(trifluoromethyl)pyrimidin-4-yl]amino]pyrazolo[1,5-a]pyridin-5-yl]-3-pyridyl]oxy]-2,2-dimethyl-propanenitrile

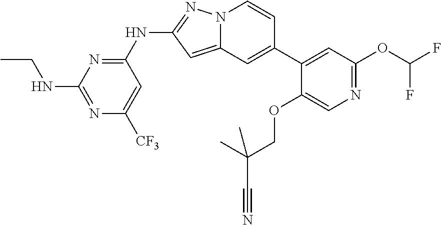 [SMILES: CCNc1nc(Nc2cc3cc(-c4cc(OC(F)F)ncc4OCC(C)(C)C#N)ccn3n2)cc(C(F)(F)F)n1]

The title compound was prepared using a similar method described in Example 502. MS (ESI): mass calcd. for C₂₅H₂₃F₅N₈O₂, 562.2. m/z found, 563.2 [M+H]⁺. ¹H NMR (600 MHz, CDCl₃) δ 8.42 (d, J=7.3 Hz, 1H), 7.88 (s, 1H), 7.68 (s, 1H), 7.54-7.28 (m, 1H), 7.08 (s, 1H), 7.00 (s, 1H), 6.57 (s, 1H), 3.97 (s, 2H), 3.51 (d, J=5.6 Hz, 2H), 2.70-2.66 (m, 3H), 1.41 (s, 6H), 1.33 (s, 3H).

### Example 701: 2-[[5-[5-(2-cyano-2-methyl-propoxy)-2-(difluoromethoxy)-4-pyridyl]pyrazolo[1,5-a]pyridin-2-yl]amino]-N-(2-hydroxy-2-methyl-propyl)-6-methyl-pyridine-4-carboxamide

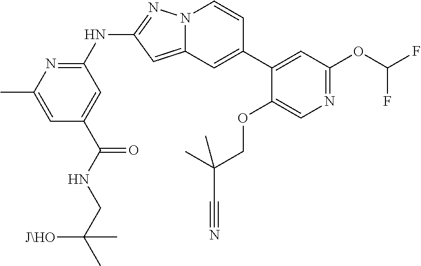

The title compound was prepared using a similar method described in Example 502. MS (ESI): mass calcd. for C₂₉H₃₁F₂N₇O₄, 579.2. m/z found, 580.2 [M+H]⁺. ¹H NMR (600 MHz, CDCl₃) δ 8.38 (dt, J=7.2, 0.9 Hz, 1H), 7.87 (s, 1H), 7.78 (s, 1H), 7.54-7.28 (m, 2H), 7.01 (s, 1H), 6.99 (dd, J=7.2, 2.0 Hz, 1H), 6.85 (s, 1H), 6.71 (s, 1H), 5.22 (s, 1H), 3.97 (s, 2H), 3.56-3.48 (m, 2H), 3.01 (d, J=7.6 Hz, 4H), 1.41 (s, 6H), 1.39-1.33 (m, 6H).

### Example 702: 3-[[6-(difluoromethoxy)-4-[2-[(1-methyl-6-oxo-pyridazin-3-yl)amino]pyrazolo[1,5-a]pyridin-5-yl]-3-pyridyl]oxy]-2,2-dimethyl-propanenitrile

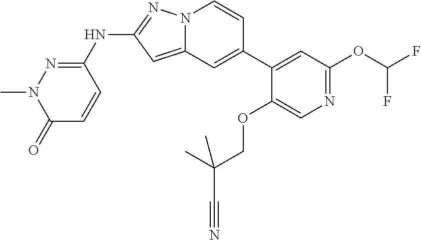 [SMILES: Cn1nc(Nc2cc3cc(-c4cc(OC(F)F)ncc4OCC(C)(C)C#N)ccn3n2)ccc1=O]

The title compound was prepared using a similar method described in Example 682, except Brettphos Pd G3 was used as the catalyst. MS (ESI): mass calcd. for C₂₃H₂₁F₂N₇O₃, 481.2. m/z found, 482.1 [M+H]⁺. ¹H NMR (400 MHz, DMSO-d₆): δ 8.57 (d, J=7.2 Hz, 1H), 8.14 (s, 1H), 7.93 (d, J=1.6 Hz, 1H), 7.67 (t, J=73.2 Hz, 1H), 7.39 (d, J=10.0 Hz, 1H), 7.27 (s, 1H), 7.07 (dd, J=2.0, 7.2 Hz, 1H), 6.93 (d, J=9.6 Hz, 1H), 6.90 (s, 1H), 4.21 (s, 2H), 3.60 (s, 3H), 1.36 (s, 6H) ¹⁹F NMR (376 MHz, DMSO-d₆): −86.43 (s, 2F)

### Example 703: 3-[[4-[2-[(2-tert-butyl-6-methyl-pyrimidin-4-yl)amino]pyrazolo[1,5-a]pyridin-5-yl]-6-(difluoromethoxy)-3-pyridyl]oxy]-2,2-dimethyl-propanenitrile

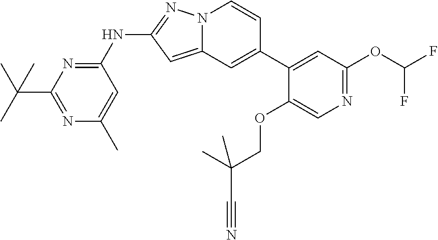 [SMILES: Cc1cc(Nc2cc3cc(-c4cc(OC(F)F)ncc4OCC(C)(C)C#N)ccn3n2)nc(C(C)(C)C)n1]

A solution of Intermediate 27 (150 mg, 0.402 mmol) and 2-(tert-butyl)-4-chloro-6-methylpyrimidine (148 mg, 0.804 mmol) in NMP (3 mL) was stirred at 120° C. for 2 hrs. The reaction was then cooled to 23° C. and purified by preparative HPLC DB using a Boston Prime C18 150×30 mm×5 μm column (eluent: 70% to 100% (v/v) water (0.05% NH₃H₂O+10 mM NH₄HCO₃)-ACN to afford pure product, which was suspended in water (10 mL), the mixture frozen using dry ice/ethanol, and then lyophilized to dryness to afford the title compound (26 mg, 12%) as a yellow solid. MS (ESI): mass calcd. for C₂₇H₂₉F₂N₇O₂, 521.2. m/z found, 522.2 [M+H]⁺. ¹H NMR (400 MHz, DMSO-d₆): δ 10.23 (br. s., 1H), 8.62 (d, J=7.2 Hz, 1H), 8.14 (s, 1H), 8.01 (s, 1H), 7.68 (t, J=73.2 Hz, 1H), 7.32 (s, 1H), 7.16-7.04 (m, 2H), 6.81 (br. s., 1H), 4.22 (s, 2H), 2.33 (s, 3H), 1.39-1.35 (m, 15H) ¹⁹F NMR (376 MHz, DMSO-d₆): −86.43 (s, 2F)

### Example 704: 3-[[4-[2-[(5-aminopyridazin-3-yl)amino]pyrazolo[1,5-a]pyridin-5-yl]-6-(difluoromethoxy)-3-pyridyl]oxy]-2,2-dimethyl-propanenitrile

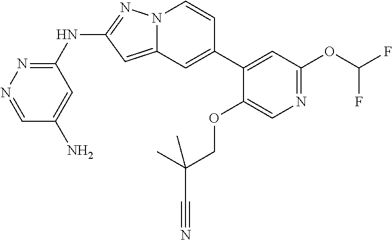 [SMILES: CC(C)(C#N)COc1cnc(OC(F)F)cc1-c1ccn2nc(Nc3cc(N)cnn3)cc2c1]

The title compound was prepared using a similar method described in Example 682, except tBuXPhos Pd G3 was used as the catalyst. MS (ESI): mass calcd. for C₂₂H₂₀F₂N₈O₂, 466.2. m/z found, 467.1 [M+H]⁺. ¹H NMR (500 MHz, CDCl₃) δ 12.76 (s, 1H), 8.38 (d, J=7.6 Hz, 1H), 7.94 (d, J=2.3 Hz, 1H), 7.87 (s, 1H), 7.67 (s, 1H), 7.55-7.39 (m, 1H), 7.04 (d, J=8.2 Hz, 1H), 6.97 (s, 1H), 6.48 (s, 1H), 3.98 (s, 2H), 1.42 (s, 6H).

### Example 705: 3-[[4-[2-[(2-aminopyrimidin-4-yl)amino]pyrazolo[1,5-a]pyridin-5-yl]-6-(difluoromethoxy)-3-pyridyl]oxy]-2,2-dimethyl-propanenitrile

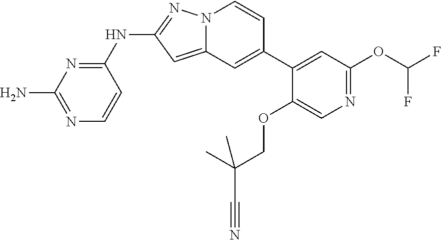 [SMILES: CC(C)(C#N)COc1cnc(OC(F)F)cc1-c1ccn2nc(Nc3ccnc(N)n3)cc2c1]

The title compound was prepared using a similar method described in Example 682, except tBuXPhos Pd G3 was used as the catalyst. MS (ESI): mass calcd. for C₂₂H₂₀F₂N₈O₂, 466.2. m/z found, 467.0 [M+H]⁺. ¹H NMR (500 MHz, CDCl₃) δ 8.41-8.35 (m, 1H), 8.07 (d, J=5.8 Hz, 1H), 7.87 (s, 1H), 7.71 (dd, J=2.0, 0.9 Hz, 1H), 7.59 (s, 1H), 7.55-7.37 (m, 1H), 7.00 (s, 1H), 6.97 (dd, J=7.2, 2.0 Hz, 1H), 6.70 (s, 1H), 6.62 (d, J=5.8 Hz, 1H), 5.00 (s, 2H), 3.96 (s, 2H), 1.41 (s, 6H).

### Example 706: 3-[[6-(difluoromethoxy)-4-[2-(4,5,6,7-tetrahydropyrazolo[1,5-a]pyrazin-2-ylamino)pyrazolo[1,5-a]pyridin-5-yl]-3-pyridyl]oxy]-2,2-dimethyl-propanenitrile

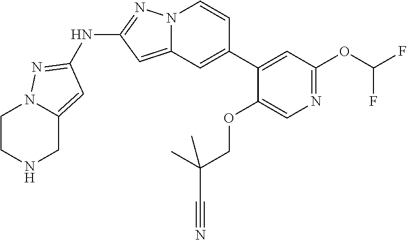 [SMILES: CC(C)(C#N)COc1cnc(OC(F)F)cc1-c1ccn2nc(Nc3cc4n(n3)CCNC4)cc2c1]

The title compound was prepared using a similar method described in Example 682, except tBuXPhos Pd G3 was used as the catalyst. MS (ESI): mass calcd. for C₂₄H₂₄F₂N₈O₂, 494.2. m/z found, 495.1 [M+H]⁺. ¹H NMR (500 MHz, CDCl₃) δ 8.33-8.25 (m, 1H), 7.86 (s, 1H), 7.58 (dd, J=1.9, 0.9 Hz, 1H), 7.56-7.36 (m, 1H), 6.98 (s, 1H), 6.84 (dd, J=7.2, 2.0 Hz, 1H), 6.68 (s, 1H), 6.34 (d, J=0.8 Hz, 1H), 5.97 (s, 1H), 4.05 (s, 2H), 4.03 (t, J=5.6 Hz, 2H), 3.94 (s, 2H), 3.32 (t, J=5.6 Hz, 2H), 1.39 (s, 6H).

### Example 707: 3-[[6-(difluoromethoxy)-4-[2-(pyrazin-2-ylamino)pyrazolo[1,5-a]pyridin-5-yl]-3-pyridyl]oxy]-2,2-dimethyl-propanenitrile

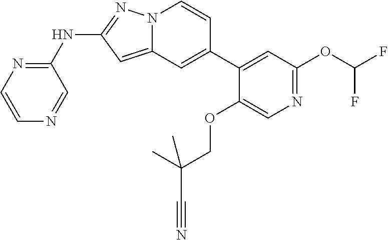 [SMILES: CC(C)(C#N)COc1cnc(OC(F)F)cc1-c1ccn2nc(Nc3cnccn3)cc2c1]

The title compound was prepared using a similar method described in Example 682, except Brettphos Pd G3 was used as the catalyst. MS (ESI): mass calcd. for C₂₂H₁₉F₂N₇O₂, 451.2. m/z found, 452.1 [M+H]⁺. ¹H NMR (400 MHz, DMSO-d₆): δ 10.33 (s, 1H), 8.67-8.60 (m, 2H), 8.23 (dd, J=1.2, 2.4 Hz, 1H), 8.14 (s, 1H), 8.02 (d, J=2.8 Hz, 1H), 7.92 (d, J=1.2 Hz, 1H), 7.68 (t, J=73.2 Hz, 1H), 7.28 (s, 1H), 7.07 (dd, J=2.0, 7.2 Hz, 1H), 6.94 (s, 1H), 4.21 (s, 2H), 1.36 (s, 6H) ¹⁹F NMR (376 MHz, DMSO-d₆): −86.49 (s, 2F)

### Example 708: 3-[[6-(difluoromethoxy)-4-[2-(pyridazin-3-ylamino)pyrazolo[1,5-a]pyridin-5-yl]-3-pyridyl]oxy]-2,2-dimethyl-propanenitrile

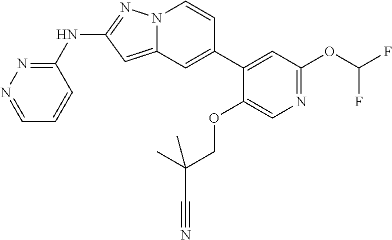 [SMILES: CC(C)(C#N)COc1cnc(OC(F)F)cc1-c1ccn2nc(Nc3cccnn3)cc2c1]

The title compound was prepared using a similar method described in Example 682, except Brettphos Pd G3 was used as the catalyst. MS (ESI): mass calcd. for C₂₂H₁₉F₂N₇O₂, 451.2. m/z found, 452.4 [M+H]⁺. ¹H NMR (400 MHz, CDCl₃): δ 9.22 (s, 1H), 8.78 (dd, J=1.2, 4.8 Hz, 1H), 8.47 (d, J=7.2 Hz, 1H), 8.12 (dd, J=1.2, 8.8 Hz, 1H), 7.87 (s, 1H), 7.69 (d, J=1.2 Hz, 1H), 7.50 (t, J=72.8 Hz, 1H), 7.46-7.41 (m, 1H), 7.01 (s, 1H), 6.98 (dd, J=2.0, 7.2 Hz, 1H), 6.73 (s, 1H), 3.97 (s, 2H), 1.41 (s, 6H) ¹⁹F NMR (376 MHz, CDCl₃): −88.29 (s, 2F)

### Example 709: 3-[[6-(difluoromethoxy)-4-[2-[(5-methylpyrazin-2-yl)amino]pyrazolo[1,5-a]pyridin-5-yl]-3-pyridyl]oxy]-2,2-dimethyl-propanenitrile

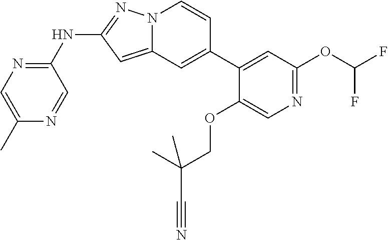 [SMILES: Cc1cnc(Nc2cc3cc(-c4cc(OC(F)F)ncc4OCC(C)(C)C#N)ccn3n2)cn1]

The title compound was prepared using a similar method described in Example 682, except Brettphos Pd G3 was used as the catalyst. MS (ESI): mass calcd. for C₂₃H₂₁F₂N₇O₂, 465.2. m/z found, 466.4 [M+H]⁺. ¹H NMR (400 MHz, CDCl₃): δ 8.67 (d, J=1.6 Hz, 1H), 8.39 (d, J=7.2 Hz, 1H), 8.09 (s, 1H), 7.87 (s, 1H), 7.69 (d, J=1.2 Hz, 1H), 7.50 (t, J=73.2 Hz, 1H), 7.44 (s, 1H), 7.00 (s, 1H), 6.95 (dd, J=1.6, 6.8 Hz, 1H), 6.70 (s, 1H), 3.96 (s, 2H), 2.50 (s, 3H), 1.41 (s, 6H) ¹⁹F NMR (376 MHz, CDCl₃): −88.30 (s, 2F)

### Example 710: 3-[[6-(difluoromethoxy)-4-[2-[(6-methyl-2-morpholino-pyrimidin-4-yl)amino]pyrazolo[1,5-a]pyridin-5-yl]-3-pyridyl]oxy]-2,2-dimethyl-propanenitrile

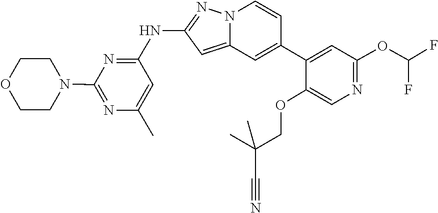 [SMILES: Cc1cc(Nc2cc3cc(-c4cc(OC(F)F)ncc4OCC(C)(C)C#N)ccn3n2)nc(N2CCOCC2)n1]

The title compound was prepared using a similar method described in Example 682, except tBuXPhos Pd G3 was used as the catalyst. MS (ESI): mass calcd. for C₂₇H₂₈F₂N₈O₃, 550.2. m/z found, 551.2 [M+H]⁺. ¹H NMR (400 MHz, DMSO-d₆): δ 10.06 (s, 1H), 8.60 (d, J=7.2 Hz, 1H), 8.13 (s, 1H), 8.05 (d, J=1.2 Hz, 1H), 7.68 (t, J=73.2 Hz, 1H), 7.32 (s, 1H), 7.08 (dd, J=2.0, 7.2 Hz, 1H), 6.96 (s, 1H), 6.27 (s, 1H), 4.22 (s, 2H), 3.72-3.65 (m, 8H), 2.18 (s, 3H), 1.37 (s, 6H)

### Example 711: 3-[[6-(difluoromethoxy)-4-[2-[(6-methylpyridazin-3-yl)amino]pyrazolo[1,5-a]pyridin-5-yl]-3-pyridyl]oxy]-2,2-dimethyl-propanenitrile

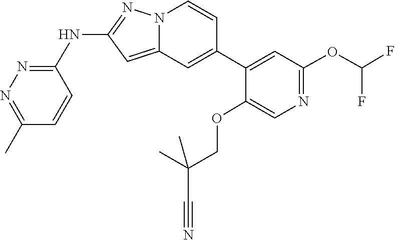 [SMILES: Cc1ccc(Nc2cc3cc(-c4cc(OC(F)F)ncc4OCC(C)(C)C#N)ccn3n2)nn1]

A solution of Intermediate 27 (100 mg, 0.268 mmol), 3-chloro-6-methylpyridazine (68 mg, 0.54 mmol), conc. HCl (0.05 mL, 0.60 mmol) in NMP (1 mL) was stirred at 140° C. in a microwave for 1 hr before cooling to 23° C. The crude mixture was purified by preparative HPLC using a Boston Prime C18 150×30 mm×5 um column (eluent: 40% to 70% (v/v) water (0.05% NH₃·H₂O+10 mM NH₄HCO₃)-ACN) to afford pure product. The product was suspended in water (10 mL), the mixture frozen using dry ice/ethanol, and then lyophilized to dryness to afford the title compound (47 mg, 37%) as a yellow solid. MS (ESI): mass calcd. for C₂₃H₂₁F₂N₇O₂, 465.2. m/z found, 466.1 [M+H]⁺. ¹H NMR (400 MHz, DMSO-d₆): δ 10.08 (s, 1H), 8.60 (d, J=7.2 Hz, 1H), 8.14 (s, 1H), 7.94 (d, J=1.2 Hz, 1H), 7.68 (t, J=73.2 Hz, 1H), 7.55 (d, J=9.2 Hz, 1H), 7.41 (d, J=9.2 Hz, 1H), 7.29 (s, 1H), 7.05 (dd, J=2.0, 7.2 Hz, 1H), 6.93 (s, 1H), 4.21 (s, 2H), 2.53-2.52 (m, 3H), 1.37 (s, 6H). ¹⁹F NMR (376 MHz, DMSO-d₆): −86.45 (s, 2F)

### Example 712: 3-[[6-(difluoromethoxy)-4-[2-[[6-(1-hydroxy-1-methyl-ethyl)pyridazin-3-yl]amino]pyrazolo[1,5-a]pyridin-5-yl]-3-pyridyl]oxy]-2,2-dimethyl-propanenitrile

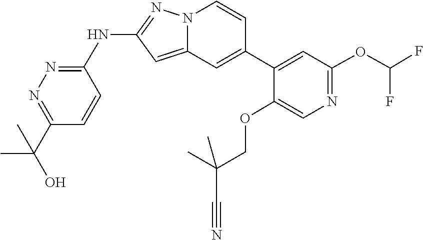 [SMILES: CC(C)(C#N)COc1cnc(OC(F)F)cc1-c1ccn2nc(Nc3ccc(C(C)(C)O)nn3)cc2c1]

The title compound was prepared using a similar method described in Example 682, except Brettphos Pd G3 was used as the catalyst. MS (ESI): mass calcd. for C₂₅H₂₅F₂N₇O₃, 509.2. m/z found, 510.4 [M+H]⁺. ¹H NMR (400 MHz, DMSO-d₆): δ 10.16 (s, 1H), 8.61 (d, J=7.2 Hz, 1H), 8.14 (s, 1H), 7.95 (d, J=1.2 Hz, 1H), 7.75 (d, J=9.2 Hz, 1H), 7.69 (t, J=73.2 Hz, 1H), 7.55 (d, J=9.2 Hz, 1H), 7.30 (s, 1H), 7.06 (dd, J=2.0, 7.2 Hz, 1H), 7.02 (s, 1H), 5.33 (s, 1H), 4.22 (s, 2H), 1.52 (s, 6H), 1.37 (s, 6H) ¹⁹F NMR (376 MHz, DMSO-d₆): −86.51 (s, 2F)

### Example 713: 3-[[6-(difluoromethoxy)-4-[2-[[5-(1-hydroxy-1-methyl-ethyl)pyrazin-2-yl]amino]pyrazolo[1,5-a]pyridin-5-yl]-3-pyridyl]oxy]-2,2-dimethyl-propanenitrile

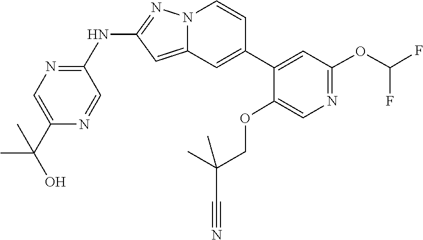 [SMILES: CC(C)(C#N)COc1cnc(OC(F)F)cc1-c1ccn2nc(Nc3cnc(C(C)(C)O)cn3)cc2c1]

The title compound was prepared using a similar method described in Example 682, except Brettphos Pd G3 was used as the catalyst. MS (ESI): mass calcd. for C₂₅H₂₅F₂N₇O₃, 509.2. m/z found, 510.1 [M+H]⁺. ¹H NMR (400 MHz, DMSO-d₆): δ 10.19 (s, 1H), 8.61 (d, J=7.2 Hz, 1H), 8.58 (d, J=1.6 Hz, 1H), 8.44 (d, J=1.2 Hz, 1H), 8.14 (s, 1H), 7.91 (d, J=0.8 Hz, 1H), 7.68 (t, J=72.8 Hz, 1H), 7.28 (s, 1H), 7.05 (dd, J=2.0, 7.6 Hz, 1H), 6.88 (s, 1H), 5.23 (s, 1H), 4.21 (s, 2H), 1.45 (s, 6H), 1.36 (s, 6H) ¹⁹F NMR (376 MHz, DMSO-d₆): −81.72 (s, 1H)

### Example 714: 3-[[6-(difluoromethoxy)-4-[2-[(1-methylpyrazol-3-yl)amino]pyrazolo[1,5-a]pyridin-5-yl]-3-pyridyl]oxy]-2,2-dimethyl-propanenitrile

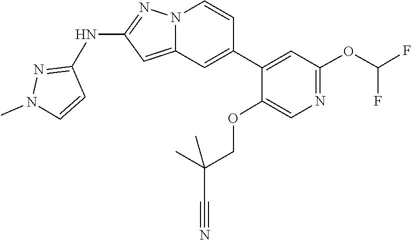 [SMILES: Cn1ccc(Nc2cc3cc(-c4cc(OC(F)F)ncc4OCC(C)(C)C#N)ccn3n2)n1]

The title compound was prepared using a similar method described in Example 682, except tBuXPhos Pd G3 was used as the catalyst. MS (ESI): mass calcd. for C₂₂H₂₁F₂N₇O₂, 453.2. m/z found, 454.3 [M+H]⁺. ¹H NMR (400 MHz, DMSO-d₆): δ 9.12 (s, 1H), 8.48 (d, J=7.2 Hz, 1H), 8.13 (s, 1H), 7.87-7.49 (m, 3H), 7.25 (s, 1H), 6.92 (dd, J=1.6, 6.8 Hz, 1H), 6.42 (s, 1H), 6.07 (d, J=2.0 Hz, 1H), 4.20 (s, 2H), 3.73 (s, 3H), 1.36 (s, 6H) ¹⁹F NMR (376 MHz, DMSO-d₆): −81.70 (s, 1H)

### Example 715: 2-[[5-[5-(2-cyano-2-methyl-propoxy)-2-(difluoromethoxy)-4-pyridyl]pyrazolo[1,5-a]pyridin-2-yl]amino]-N,6-dimethyl-pyridine-4-carboxamide

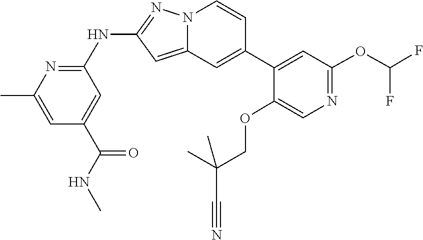 [SMILES: CNC(=O)c1cc(C)nc(Nc2cc3cc(-c4cc(OC(F)F)ncc4OCC(C)(C)C#N)ccn3n2)c1]

The title compound was prepared using a similar method described in Example 682, except Brettphos Pd G3 was used as the catalyst. MS (ESI): mass calcd. for C₂₆H₂₅F₂N₇O₃, 521.2. m/z found, 522.4 [M+H]⁺. ¹H NMR (400 MHz, DMSO-d₆): δ 9.96 (s, 1H), 8.58 (d, J=7.2 Hz, 1H), 8.51 (q, J=4.4 Hz, 1H), 8.14 (s, 1H), 7.90 (d, J=1.2 Hz, 1H), 7.67 (t, J=73.2 Hz, 1H), 7.48 (s, 1H), 7.28 (s, 1H), 7.03 (dd, J=2.0, 7.2 Hz, 1H), 6.99 (s, 1H), 6.93 (s, 1H), 4.21 (s, 2H), 2.78 (d, J=4.4 Hz, 3H), 2.46 (s, 3H), 1.36 (s, 6H) ¹⁹F NMR (376 MHz, DMSO-d₆): −86.44 (s, 2F)

### Example 716: 3-[[6-(difluoromethoxy)-4-[2-(5,6,7,8-tetrahydro-2,7-naphthyridin-3-ylamino)pyrazolo[1,5-a]pyridin-5-yl]-3-pyridyl]oxy]-2,2-dimethyl-propanenitrile

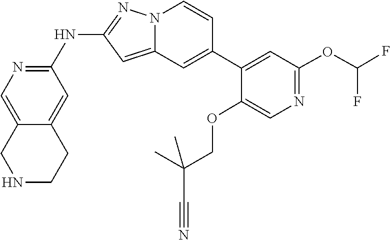 [SMILES: CC(C)(C#N)COc1cnc(OC(F)F)cc1-c1ccn2nc(Nc3cc4c(cn3)CNCC4)cc2c1]

The title compound was prepared using a similar method described in Example 682, except Brettphos Pd G3 was used as the catalyst. MS (ESI): mass calcd. for C₂₆H₂₅F₂N₇O₂, 505.2. m/z found, 506.2 [M+H]⁺. ¹H NMR (400 MHz, DMSO-d₆): δ 9.57 (s, 1H), 8.55 (d, J=7.2 Hz, 1H), 8.13 (s, 1H), 7.91 (s, 1H), 7.87-7.48 (m, 2H), 7.26 (s, 1H), 7.08 (s, 1H), 6.98 (dd, J=2.0, 7.2 Hz, 1H), 6.79 (s, 1H), 4.21 (s, 2H), 3.78 (s, 2H), 2.92 (t, J=5.6 Hz, 2H), 2.66 (t, J=5.6 Hz, 2H), 1.36 (s, 6H) ¹⁹F NMR (376 MHz, DMSO-d₆): −86.46 (s, 2F)

### Example 717: 3-[[6-(difluoromethoxy)-4-[2-[(2-methylpyrimidin-4-yl)amino]pyrazolo[1,5-a]pyridin-5-yl]-3-pyridyl]oxy]-2,2-dimethyl-propanenitrile

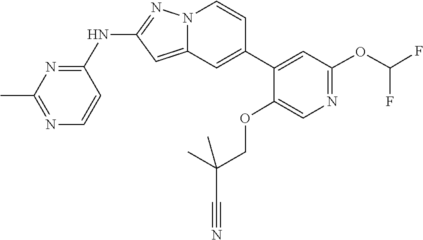 [SMILES: Cc1nccc(Nc2cc3cc(-c4cc(OC(F)F)ncc4OCC(C)(C)C#N)ccn3n2)n1]

The title compound was prepared using a similar method described in Example 682, except tBuXPhos Pd G3 was used as the catalyst. MS (ESI): mass calcd. for C₂₃H₂₁F₂N₇O₂, 465.2. m/z found, 466.2 [M+H]⁺. ¹H NMR (400 MHz, DMSO-d₆) δ 10.28 (s, 1H), 8.55 (dt, J=7.2, 0.9 Hz, 1H), 8.21 (d, J=5.9 Hz, 1H), 8.08 (s, 1H), 7.87 (dd, J=2.0, 0.9 Hz, 1H), 7.60 (t, J=73.2 Hz, 1H), 7.21 (s, 1H), 7.11 (s, 1H), 7.00 (dd, J=7.2, 2.0 Hz, 1H), 6.82 (s, 1H), 4.14 (s, 2H), 1.29 (s, 6H).

### Example 718: 3-[[6-(difluoromethoxy)-4-[2-(5,6,7,8-tetrahydro-2,6-naphthyridin-3-ylamino)pyrazolo[1,5-a]pyridin-5-yl]-3-pyridyl]oxy]-2,2-dimethyl-propanenitrile

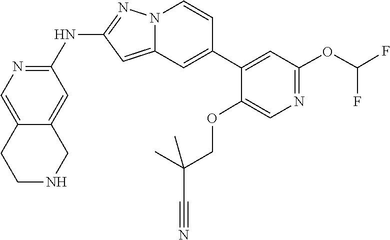 [SMILES: CC(C)(C#N)COc1cnc(OC(F)F)cc1-c1ccn2nc(Nc3cc4c(cn3)CCNC4)cc2c1]

The title compound was prepared using a similar method described in Example 682, except Brettphos Pd G3 was used as the catalyst. MS (ESI): mass calcd. for C₂₆H₂₅F₂N₇O₂, 505.2. m/z found, 506.1 [M+H]⁺. ¹H NMR (400 MHz, DMSO-d₆): δ 9.55 (s, 1H), 8.55 (d, J=7.2 Hz, 1H), 8.13 (s, 1H), 7.95 (s, 1H), 7.84 (s, 1H), 7.67 (t, J=73.2 Hz, 1H), 7.26 (s, 1H), 7.02 (s, 1H), 6.98 (dd, J=1.6, 7.2 Hz, 1H), 6.78 (s, 1H), 4.21 (s, 2H), 3.80 (s, 2H), 2.93 (t, J=5.6 Hz, 2H), 2.60 (t, J=5.6 Hz, 2H), 1.36 (s, 6H) ¹⁹F NMR (376 MHz, DMSO-d₆): −86.48 (s, 2F)

### Example 719: 3-[[6-(difluoromethoxy)-4-[2-(5,6,7,8-tetrahydro-1,7-naphthyridin-2-ylamino)pyrazolo[1,5-a]pyridin-5-yl]-3-pyridyl]oxy]-2,2-dimethyl-propanenitrile

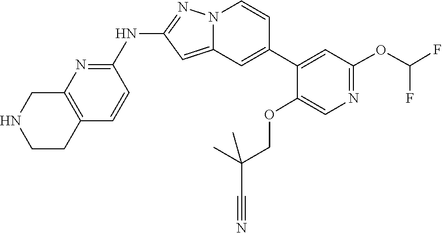 [SMILES: CC(C)(C#N)COc1cnc(OC(F)F)cc1-c1ccn2nc(Nc3ccc4c(n3)CNCC4)cc2c1]

The title compound was prepared using a similar method described in Example 682, except Brettphos Pd G3 was used as the catalyst. MS (ESI): mass calcd. for C₂₆H₂₅F₂N₇O₂, 505.2. m/z found, 506.1 [M+H]⁺. ¹H NMR (400 MHz, DMSO-d₆): δ 9.68 (s, 1H), 8.55 (d, J=7.2 Hz, 1H), 8.13 (s, 1H), 7.86 (s, 1H), 7.68 (t, J=73.2 Hz, 1H), 7.34 (d, J=8.4 Hz, 1H), 7.28 (s, 1H), 7.17 (d, J=8.4 Hz, 1H), 6.99 (dd, J=2.0, 7.2 Hz, 1H), 6.82 (s, 1H), 4.21 (s, 2H), 3.79 (s, 2H), 2.94 (t, J=5.6 Hz, 2H), 2.61 (t, J=5.0 Hz, 2H), 1.36 (s, 6H) ¹⁹F NMR (376 MHz, DMSO-d₆): −86.48 (d, J=73.32 Hz, 2F)

### Example 720: 1-[[6-(difluoromethoxy)-4-[2-[(2,6-dimethylpyrimidin-4-yl)amino]pyrazolo[1,5-a]pyridin-5-yl]-3-pyridyl]oxymethyl]cyclopropanecarbonitrile

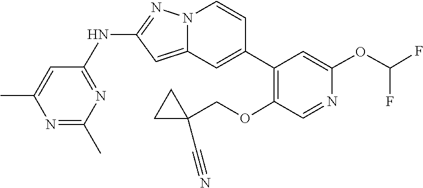 [SMILES: Cc1cc(Nc2cc3cc(-c4cc(OC(F)F)ncc4OCC4(C#N)CC4)ccn3n2)nc(C)n1]

The title compound was prepared using a similar method described in Example 329. MS (ESI): mass calcd. for C₂₄H₂₁F₂N₇O₂, 477.2. m/z found, [M+H]⁺. ¹H NMR (400 MHz, DMSO-d₆): δ 10.25 (s, 1H), 8.62 (d, J=7.2 Hz, 1H), 8.08 (s, 1H), 7.97 (s, 1H), 7.67 (t, J=73.2 Hz, 1H), 7.28 (s, 1H), 7.11 (dd, J=1.6, 7.2 Hz, 1H), 7.02 (br s, 1H), 6.91 (br s, 1H), 4.21 (s, 2H), 2.46 (s, 3H), 2.31 (s, 3H), 1.38-1.32 (m, 2H), 1.17-1.12 (m, 2H) ¹⁹F NMR (376 MHz, DMSO-d₆): −86.50 (s, 2F)

### Example 721: (*R)-2-[[4-[2-[(2,6-dimethylpyrimidin-4-yl)amino]pyrazolo[1,5-a]pyridin-5-yl]-5-(4-hydroxycyclohexoxy)-2-pyridyl]oxy]propanenitrile

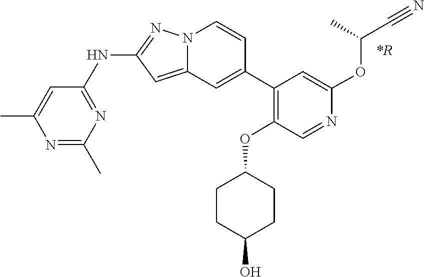 [SMILES: Cc1cc(Nc2cc3cc(-c4cc(O[C@H](C)C#N)ncc4O[C@H]4CC[C@H](O)CC4)ccn3n2)nc(C)n1]

The title compound was prepared using a similar method described in Example 672. The material was separated by SFC with DAICEL CHIRALPAK AD (250 mm×50 mm, 10 um), column (eluent: 50% to 50% (v/v) IPA with 0.1% NH₃·H₂O in supercritical CO₂) to afford pure product. The title compound was the first to elute. MS (ESI): mass calcd. for C₂₇H₂₉N₇O₃, 499.2. m/z found, 500.1 [M+H]⁺. ¹H NMR (400 MHz, CDCl₃): δ 8.36 (d, J=7.2 Hz, 1H), 7.94 (s, 1H), 7.62 (s, 1H), 7.41 (s, 1H), 7.07 (s, 1H), 6.97-6.92 (m, 1H), 6.86 (s, 1H), 6.60 (s, 1H), 5.64 (q, J=6.8 Hz, 1H), 4.20-4.11 (m, 1H), 3.79-3.69 (m, 1H), 2.61 (s, 3H), 2.45 (s, 3H), 2.12-2.00 (m, 2H), 1.96-1.86 (m, 2H), 1.78 (d, J=6.8 Hz, 3H), 1.57-1.48 (m, 2H), 1.41-1.33 (m, 2H)

### Example 722: 3-[2-[[4-[2-[(2,6-dimethylpyrimidin-4-yl)amino]pyrazolo[1,5-a]pyridin-5-yl]-6-methyl-3-pyridyl]oxy]ethyl]oxetane-3-carbonitrile

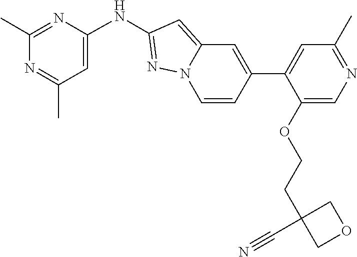 [SMILES: Cc1cc(-c2ccn3nc(Nc4cc(C)nc(C)n4)cc3c2)c(OCCC2(C#N)COC2)cn1]

The title compound was prepared using a similar method described in Example 329. In Step A 4-iodo-6-methylpyridin-3-ol was used instead of 4-iodo-6-methoxypyridin-3-ol. MS (ESI): mass calcd. for C₂₅H₂₅N₇O₂, 455.2. m/z found, 456.1 [M+H]⁺. ¹H NMR (400 MHz, CDCl₃): δ 8.40 (d, J=7.2 Hz, 1H), 8.29 (s, 1H), 7.60 (s, 1H), 7.51 (d, J=0.8 Hz, 1H), 7.13 (s, 1H), 7.10 (s, 1H), 6.81 (dd, J=1.6, 7.2 Hz, 1H), 6.60 (s, 1H), 4.77 (d, J=6.4 Hz, 2H), 4.46 (d, J=6.4 Hz, 2H), 4.32 (t, J=5.2 Hz, 2H), 2.61 (s, 3H), 2.57 (s, 3H), 2.48 (t, J=5.6 Hz, 2H), 2.46 (s, 3H)

### Example 723: N-[5-[2-(difluoromethoxy)-5-(4-hydroxycyclohexoxy)-4-pyridyl]pyrazolo[1,5-a]pyridin-2-yl]cyclopropanecarboxamide

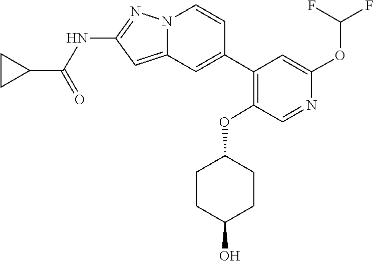 [SMILES: O=C(Nc1cc2cc(-c3cc(OC(F)F)ncc3O[C@H]3CC[C@H](O)CC3)ccn2n1)C1CC1]

Step A. N-(5-(2-chloro-5-(((1r,4r)-4-((tetrahydro-2H-pyran-2-yl)oxy)cyclohexyl)oxy)pyridin-4-yl)pyrazolo[1,5-a]pyridin-2-yl)cyclopropanecarboxamide. NaH (1.11 g, 27.8 mmol) was added to a solution of N-(5-(2-chloro-5-fluoropyridin-4-yl)pyrazolo[1,5-a]pyridin-2-yl)cyclopropanecarboxamide (3.1 g, 9.3 mmol), 3-hydroxy-2,2-dimethylpropanenitrile (2.6 g, 13 mmol), and DMA (50 mL) at 0° C. Then the reaction mixture was stirred for 1 h at room temperature. The mixture was poured into aq. Sat. NH₄Cl (100 mL) and the suspension isolated via filtration. The filter cake was washed with water (30 mL×3) before drying under reduced pressure to afford the title compound (6 g, crude) as a yellow solid.

Step B. N-(5-(2-hydroxy-5-(((1r,4r)-4-((tetrahydro-2H-pyran-2-yl)oxy)cyclohexyl)oxy)pyridin-4-yl)pyrazolo[1,5-a]pyridin-2-yl)cyclopropanecarboxamide. A mixture consisting of N-(5-(2-chloro-5-(((1r,4r)-4-((tetrahydro-2H-pyran-2-yl)oxy)cyclohexyl)oxy)pyridin-4-yl)pyrazolo[1,5-a]pyridin-2-yl)cyclopropanecarboxamide (3.0 g, 5.9 mmol), Pd₂(dba)₃ (538 mg, 0.587 mmol), t-BuXphos (249 mg, 0.587 mmol), KOH (1.65 g, 29.4 mmol), 1,4-dioxane (20 mL), and H₂O (5 mL) was stirred at 100° C. for 2 h under N₂. The reaction mixture was directly concentrated to afford the crude product, which was purified by FCC (EA:EE=1:0 to 1:1) to afford the title compound (1 g, 24%) as a yellow solid.

Step C. N-(5-(2-(difluoromethoxy)-5-4(1r,4r)-4-((tetrahydro-2H-pyran-2-yl)oxy)cyclohexyl)oxy)pyridin-4-yl)pyrazolo[1,5-a]pyridin-2-yl)cyclopropanecarboxamide. A mixture consisting of N-(5-(2-hydroxy-5-(((1r,4r)-4-((tetrahydro-2H-pyran-2-yl)oxy)cyclohexyl)oxy)pyridin-4-yl)pyrazolo[1,5-a]pyridin-2-yl)cyclopropanecarboxamide (150 mg, 0.305 mmol), diethyl (bromodifluoromethyl)phosphonate (81.3 mg, 0.305 mmol), KF (35.4 mg, 0.609 mmol), and ACN (3 mL) was stirred for overnight at room temperature. The reaction mixture was purified by preparative HPLC using a Boston Prime C18 150×30 mm×5 μm column (eluent: 70% to 100% (v/v) CH₃CN and H₂O with 0.05% NH₃H₂O and 10 mM NH₄HCO₃) to afford pure product. The product was suspended in water (10 mL), the mixture frozen using dry ice/ethanol, and then lyophilized to dryness to afford the title compound (40 mg, 24%) as a yellow solid.

Step D. N-[5-[2-(difluoromethoxy)-5-(4-hydroxycyclohexoxy)-4-pyridyl]pyrazolo[1,5-a]pyridin-2-yl]cyclopropanecarboxamide. A solution consisting of N-(5-(2-(difluoromethoxy)-5-(((1r,4r)-4-((tetrahydro-2H-pyran-2-yl)oxy)cyclohexyl)oxy)pyridin-4-yl)pyrazolo[1,5-a]pyridin-2-yl)cyclopropanecarboxamide (25 mg, 0.046 mmol), TFA (0.6 mL), and CH₂Cl₂ (2 mL) was stirred for 1 h at room temperature. The reaction mixture was directly concentrated to afford the crude product, which was purified by preparative HPLC using a Phenomenex Gemini-NX C18 75×30 mm×3 μm column (eluent: 37% to 67% (v/v) CH₃CN and H₂O with 0.05% NH₃ and 10 mM NH₄HCO₃) to afford pure product. The product was suspended in water (10 mL), the mixture frozen using dry ice/ethanol, and then lyophilized to dryness to afford the title compound (5.2 mg, 25%) as a yellow solid. MS (ESI): mass calcd. for C₂₃H₂₄F₂N₄O₄, 458.2. m/z found, 459.1 [M+H]⁺. ¹H NMR (400 MHz, DMSO-d₆): δ 11.11 (s, 1H), 8.58 (d, J=7.2 Hz, 1H), 8.16 (s, 1H), 7.89-7.85 (m, 1H), 7.64 (t, J=73.2 Hz, 1H), 7.22 (s, 1H), 7.05 (dd, J=2.0, 7.2 Hz, 1H), 6.91 (s, 1H), 4.53 (d, J=4.0 Hz, 1H), 4.48-4.36 (m, 1H), 3.56-3.44 (m, 1H), 2.02-1.89 (m, 3H), 1.78-1.68 (m, 2H), 1.47-1.35 (m, 2H), 1.33-1.22 (m, 2H), 0.86-0.79 (m, 4H) ¹⁹F NMR (376 MHz, DMSO-d₆): −86.23 (s, 2F)

### Example 724: N-[5-[2-(cyanomethoxy)-5-(2-cyano-2-methyl-propoxy)-4-pyridyl]pyrazolo[1,5-a]pyridin-2-yl]cyclopropanecarboxamide

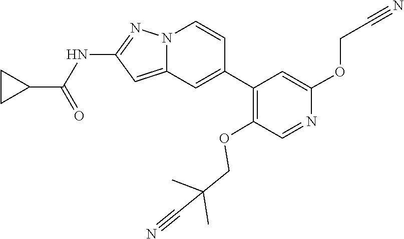 [SMILES: CC(C)(C#N)COc1cnc(OCC#N)cc1-c1ccn2nc(NC(=O)C3CC3)cc2c1]

The title compound was prepared using a similar manner as described in Example 259 Step B, except 3-((6-(cyanomethoxy)-4-iodopyridin-3-yl)oxy)-2,2-dimethylpropanenitrile and N-(5-(4,4,5,5-tetramethyl-1,3,2-dioxaborolan-2-yl)pyrazolo[1,5-a]pyridin-2-yl)cyclopropanecarboxamide were used. MS (ESI): mass calcd. for C₂₃H₂₂N₆O₃, 430.2. m/z found, 431.5 [M+H]⁺. ¹H NMR (400 MHz, DMSO-d₆): δ 11.14 (s, 1H), 8.60 (d, J=7.2 Hz, 1H), 8.11 (s, 1H), 7.93 (s, 1H), 7.15 (s, 1H), 7.08 (dd, J=2.0, 7.2 Hz, 1H), 6.90 (s, 1H), 5.22 (s, 2H), 4.18 (s, 2H), 2.00-1.82 (m, 1H), 1.35 (s, 6H), 0.85-0.79 (m, 4H)

### Example 725: 3-[[6-(2,2-difluoroethoxy)-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile

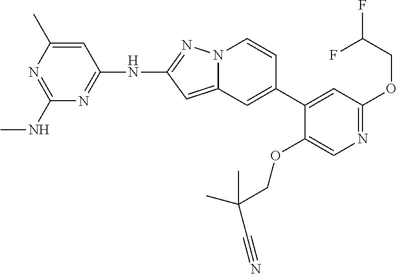 [SMILES: CNc1nc(C)cc(Nc2cc3cc(-c4cc(OCC(F)F)ncc4OCC(C)(C)C#N)ccn3n2)n1]

The title compound was prepared using a similar manner as described in Example 259 Step B, except Intermediate 28 and 4-chloro-N,6-dimethylpyrimidin-2-amine were used. MS (ESI): mass calcd. for C₂₅H₂₆F₂N₈O₂, 508.2. m/z found, 509.7 [M+H]⁺. ¹H NMR (400 MHz, DMSO-d₆): δ 9.88 (s, 1H), 8.55 (d, J=7.2 Hz, 1H), 8.05 (s, 1H), 7.96-7.81 (m, 1H), 7.10 (s, 1H), 7.08 (s, 1H), 7.02 (dd, J=1.2, 7.2 Hz, 1H), 6.64 (br. s., 1H), 6.57-6.25 (m, 1H), 6.22 (br. s., 1H), 4.65-4.48 (m, 2H), 4.14 (s, 2H), 2.84 (d, J=4.0 Hz, 3H), 2.13 (s, 3H), 1.35 (s, 6H) ¹⁹F NMR (376 MHz, DMSO-d₆): −125.36-−125.81 (m, 2F)

### Example 726: (*S)—N-[5-[2-(1-cyanoethoxy)-5-(2-cyano-2-methyl-propoxy)-4-pyridyl]pyrazolo[1,5-a]pyridin-2-yl]cyclopropanecarboxamide

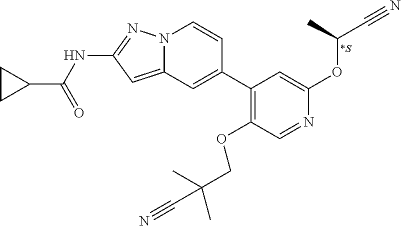 [SMILES: C[C@@H](C#N)Oc1cc(-c2ccn3nc(NC(=O)C4CC4)cc3c2)c(OCC(C)(C)C#N)cn1]

The title compound was prepared as described in Example 680 except Intermediate 3 was used. The enantiomeric mixture was separated by SFC with DAICEL CHIRALPAK AD (250 mm×30 mm, 10 um), column (eluent: 50% to 50% (v/v) EtOH with 0.1% NH₃·H₂O in supercritical CO₂) to afford pure product. The title compound was second to elute. MS (ESI): mass calcd. for C₂₄H₂₄N₆O₃, 444.2. m/z found, 445.4 [M+H]⁺. ¹H NMR (400 MHz, DMSO-d₆): δ 11.15 (s, 1H), 8.60 (d, J=7.2 Hz, 1H), 8.12 (s, 1H), 7.94 (s, 1H), 7.13 (s, 1H), 7.09 (dd, J=1.6, 7.2 Hz, 1H), 6.89 (s, 1H), 5.72 (q, J=6.4 Hz, 1H), 4.18 (s, 2H), 1.98-1.89 (m, 1H), 1.71 (d, J=6.8 Hz, 3H), 1.35 (s, 6H), 0.87-0.79 (m, 4H)

### Example 727: 3-[[6-(difluoromethoxy)-4-[2-[(2,6-dimethylpyrimidin-4-yl)amino]pyrazolo[1,5-a]pyridin-5-yl]-3-pyridyl]oxy]-2,2-dimethyl-propanamide

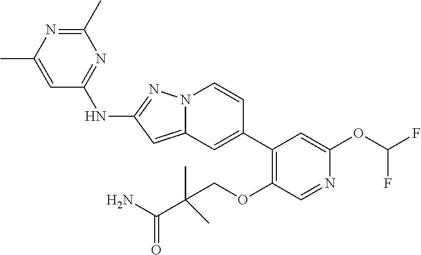 [SMILES: Cc1cc(Nc2cc3cc(-c4cc(OC(F)F)ncc4OCC(C)(C)C(N)=O)ccn3n2)nc(C)n1]

Step A: methyl 2,2-dimethyl-3-(tosyloxy)propanoate. A solution consisting of methyl 3-hydroxy-2,2-dimethylpropanoate (5.0 g, 38 mmol), DMAP (6.9 g, 57 mmol), and DCM (60 mL) was stirred in ice bath at 0° C. To the solution was slowly added TsCl (7.9 g, 42 mmol) in batches. The resultant mixture was stirred overnight at room-temperature. The reaction mixture was washed with 1N HCl (50 mL×2), saturated aqueous NaHCO₃ (50 mL×2) and concentrated under reduced pressure to afford the title compound as a white solid (10 g, 92%), which was purified by preparative HPLC using a Welch Xtimate C18 250×50 mm×10 um column (eluent: 30% to 65% (v/v) water (10 mM NH₄HCO₃)-ACN) to afford pure product. The product was suspended in water (10 mL), the mixture frozen using dry ice/ethanol, and then lyophilized to dryness to afford the title compound (4.5 g, 42%) as a white solid.

Step B: methyl 3-((6-(difluoromethoxy)-4-(2-((2,6-dimethylpyrimidin-4-yl)amino)pyrazolo[1,5-a]pyridin-5-yl)pyridin-3-yl)oxy)-2,2-dimethylpropanoate. The title compound was prepared as described in Example 329, except methyl 2,2-dimethyl-3-(tosyloxy)propanoate was used instead of 2-cyano-2-methylpropyl-4-methylbenzenesulfonate in Step A.

Step C. 3-((6-(difluoromethoxy)-4-(2-((2,6-dimethylpyrimidin-4-yl)amino)pyrazolo[1,5-a]pyridin-5-yl)pyridin-3-yl)oxy)-2,2-dimethylpropanoic acid. A mixture consisting of methyl 3-((6-(difluoromethoxy)-4-(2-((2,6-dimethylpyrimidin-4-yl)amino)pyrazolo[1,5-a]pyridin-5-yl)pyridin-3-yl)oxy)-2,2-dimethylpropanoate (3.7 g, 7.2-mmol), LiOH·H₂O-(3.0 g, −72-mmol), THF-(20 mL), and H₂O (5 mL) was stirred at 60° C. for 3 hr before cooling to room temperature. The mixture was poured into water (50 mL), then extracted with ethyl acetate (50 mL×3). The combined organic layers were dried over Na₂SO₄. The organics were concentrated under reduce pressure to afford the desired compound (3.9 g, crude) as a white solid.

Step D: 3-[[6-(difluoromethoxy)-4-[2-[(2,6-dimethylpyrimidin-4-yl)amino]pyrazolo[1,5-a]pyridin-5-yl]-3-pyridyl]oxy]-2,2-dimethyl-propanamide. A-mixture consisting of 34(6-(difluoromethoxy)-4-(2-((2,6-dimethylpyrimidin-4-yl)amino)pyrazolo[1,5-a]pyridin-5-yl)pyridin-3-yl)oxy)-2,2-dimethylpropanoic acid (100-mg, 0.201 mmol), NH₃·H₂O (37-mg, −0.26-mmol), DIEA (130-mg, −1.00-mmol), HATU (153-mg, 0.401-mmol) and DMF (2-mL) was stirred at 25° C. for 1 h before cooling to room temperature. The crude product was further purified by preparative HPLC using a Phenomenex Gemini-NX 75×30 mm×3 um column (eluent: 33% to 63% (v/v) CH₃CN and H₂O with 0.05% NH₃H₂O+10 mM NH₄HCO₃) to afford product. The product was suspended in water (10 mL), the mixture frozen using dry ice/ethanol, and then lyophilized to dryness to afford the title compound (28 mg, 28%) as white solid. MS (ESI): mass calcd. for C₂₄H₂₅F₂N₇O₃, 497.2. m/z found, 498.3 [M+H]⁺. ¹H NMR (400 MHz, DMSO-d₆): δ 10.23 (s, 1H), 8.57 (d, J=7.2 Hz, 1H), 8.09 (s, 1H), 7.85 (d, J=1.2 Hz, 1H), 7.65 (t, J=73.2 Hz, 1H), 7.23 (s, 1H), 7.19 (s, 1H), 7.10-6.99 (m, 3H), 6.87 (s, 1H), 4.12 (s, 2H), 2.47 (s, 3H), 2.31 (s, 3H), 1.15 (s, 6H) ¹⁹F NMR (376 MHz, DMSO-d₆): −86.29 (s, 2F)

### Example 728: 3-[[6-(difluoromethoxy)-4-[2-[(2,6-dimethylpyrimidin-4-yl)amino]pyrazolo[1,5-a]pyridin-5-yl]-3-pyridyl]oxy]-N,N,2,2-tetramethyl-propanamide

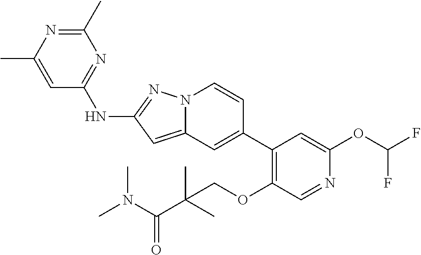 [SMILES: Cc1cc(Nc2cc3cc(-c4cc(OC(F)F)ncc4OCC(C)(C)C(=O)N(C)C)ccn3n2)nc(C)n1]

The title compound (74 mg, 34%) was prepared as described in Example 727, except dimethylamine hydrochloride was used in Step D. MS (ESI): mass calcd. for C₂₆H₂₉F₂N703, 525.2. m/z found, 526.3 [M+H]⁺. ¹H NMR (400 MHz, DMSO-d₆): δ 10.24 (s, 1H), 8.57 (d, J=7.2 Hz, 1H), 8.10 (s, 1H), 7.85 (d, J=0.8 Hz, 1H), 7.66 (t, J=1.2 Hz, 1H), 7.23 (s, 1H), 7.06-7.01 (m, 1H), 6.99 (dd, J=2.0, 7.2 Hz, 1H), 6.89 (s, 1H), 4.19 (s, 2H), 2.95 (s, 6H), 2.46 (s, 3H), 2.31 (s, 3H), 1.26 (s, 6H) ¹⁹F NMR (376 MHz, DMSO-d₆-86.32 (s, 2F)

### Example 729: 3-[[6-(difluoromethoxy)-4-[2-[(2,6-dimethylpyrimidin-4-yl)amino]imidazo[1,2-a]pyrazin-6-yl]-3-pyridyl]oxy]-2,2-dimethyl-propanenitrile

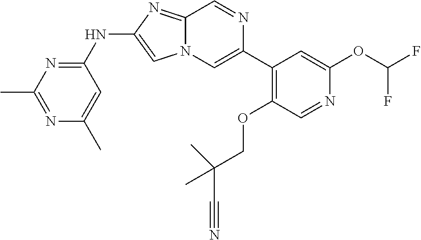 [SMILES: Cc1cc(Nc2cn3cc(-c4cc(OC(F)F)ncc4OCC(C)(C)C#N)ncc3n2)nc(C)n1]

Step A: (5-(2-cyano-2-methylpropoxy)-2-(difluoromethoxy)pyridin-4-yl)boronic acid. To a round bottom flask, i-PrMgCl·LiCl (8.36 mL, 10.9 mmol, 1.3 M in THF) was added drop-wise over 15 min to a −65° C. (dry ice/EtOH) solution consisting of 3-((6-(difluoromethoxy)-4-iodopyridin-3-yl)oxy)-2,2-dimethylpropanenitrile (1.00 g, 2.72 mmol) and THF (10 mL) and stirred at −65° C. for 30 min. Then 2-isopropoxy-4,4,5,5-tetramethyl-1,3,2-dioxaborolane (3.54 g, 19.0 mmol) was added drop-wise over 5 min to the reaction mixture under N₂ at −65° C. The mixture was warmed to room temperature and stirred overnight. The reaction mixture was poured into aq. Sat. NH₄Cl (50 mL) and extracting with EtOAc (50 mL×3). The combined organic extracts were concentrated to dryness under reduced pressure to give the crude product, which was purified by preparative HPLC using a Phenomenex DE Boston Uni C18 150×40 mm×5 um column (eluent: 30% to 60% (v/v) water (0.225% FA)-ACN) to afford pure product. The product was suspended in water (10 mL), the mixture frozen using dry ice/ethanol, and then lyophilized to dryness to afford the title compound (160 mg, 21%) as a yellow solid.

Step B: 3-[[6-(difluoromethoxy)-4-[2-[(2,6-dimethylpyrimidin-4-yl)amino]imidazo[1,2-a]pyrazin-6-yl]-3-pyridyl]oxy]-2,2-dimethyl-propanenitrile. The title compound was prepared as described in Example 259 Steps B-C, except (5-(2-cyano-2-methylpropoxy)-2-(difluoromethoxy)pyridin-4-yl)boronic acid was used and Brettphos-Pd-G3 was used as the catalyst in Step D (36.8 mg, 30%). MS (ESI): mass calcd. for C₂₃H₂₂F₂N₈O₂, 480.2. m/z found, 481.1 [M+H]⁺. ¹H NMR (400 MHz, DMSO-d₆): δ 10.52 (s, 1H), 9.44 (d, J=1.2 Hz, 1H), 9.01 (s, 1H), 8.51 (s, 1H), 8.20 (s, 1H), 7.86-7.45 (m, 2H), 6.73 (s, 1H), 4.35 (s, 2H), 2.48 (s, 3H), 2.30 (s, 3H), 1.46 (s, 6H) ¹⁹F NMR (376 MHz, DMSO-d₆): −86.23 (s, 2F)

### Example 730: 3-[[4-[2-[(1-cyclopropyl-5-methyl-6-oxo-pyridazin-3-yl)amino]pyrazolo[1,5-a]pyridin-5-yl]-6-(2,2-difluoroethoxy)-3-pyridyl]oxy]-2,2-dimethyl-propanenitrile

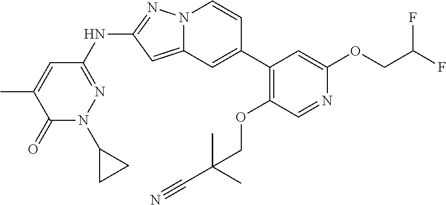 [SMILES: Cc1cc(Nc2cc3cc(-c4cc(OCC(F)F)ncc4OCC(C)(C)C#N)ccn3n2)nn(C2CC2)c1=O]

The title compound (43.2 mg, 20%) was prepared as described in Example 688, except Brettphos-Pd-G3 was used as the catalyst. MS (ESI): mass calcd. for C₂₇H₂₇F₂N₇O₃, 535.2. m/z found, 536.5 [M+H]⁺. ¹H NMR (400 MHz, DMSO-d₆): δ 9.82 (s, 1H), 8.54 (d, J=7.2 Hz, 1H), 8.05 (s, 1H), 7.91 (d, J=1.2 Hz, 1H), 7.23 (d, J=1.6 Hz, 1H), 7.10 (s, 1H), 7.05 (dd, J=2.0, 7.2 Hz, 1H), 6.86 (s, 1H), 6.57-6.25 (m, 1H), 6.05-5.94 (m, 1H), 5.30-5.21 (m, 2H), 4.65-4.52 (m, 4H), 4.14 (s, 2H), 2.10 (d, J=1.2 Hz, 3H), 1.35 (s, 6H) ¹⁹F NMR (376 MHz, DMSO-d₆): −125.53 (s, 2F)

### Example 731: (*R)-4-[[6-(difluoromethoxy)-4-[2-[(2,6-dimethylpyrimidin-4-yl)amino]pyrazolo[1,5-a]pyridin-2-yl]-3-pyridyl]oxymethyl]-5,5-dimethyl-pyrrolidin-2-one

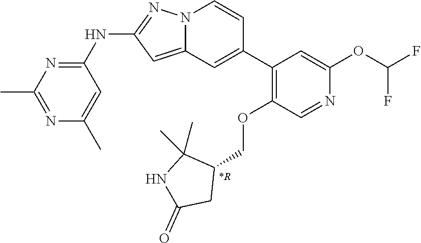 [SMILES: Cc1cc(Nc2cc3cc(-c4cc(OC(F)F)ncc4OC[C@@H]4CC(=O)NC4(C)C)ccn3n2)nc(C)n1]

The title compound (85 mg, 20%) was prepared as described in Example 502 Steps A-B, except 4-(hydroxymethyl)-5,5-dimethylpyrrolidin-2-one was used. The enantiomeric mixture was purified by SFC-16 over DAICEL CHIRALCEL OJ-H 250 mm×30 mm×5 μm (eluent: 45% to 45% (v/v) MeOH with 0.1% NH₃·H₂O in supercritical CO₂). The title compound was first to elute. MS (ESI): mass calcd. for C₂₆H₂₇F₂N₇O₃, 523.2. m/z found, 524.6 [M+H]⁺. ¹H NMR (400 MHz, DMSO-d₆): δ 10.22 (s, 1H), 8.61 (d, J=7.2 Hz, 1H), 8.17 (s, 1H), 7.85-7.46 (m, 3H), 7.22 (s, 1H), 7.07-7.01 (m, 1H), 7.00 (dd, J=1.6, 7.2 Hz, 1H), 6.88 (br. s., 1H), 4.27-4.20 (m, 1H), 4.17-4.09 (m, 1H), 2.46 (s, 3H), 2.31 (s, 3H), 2.28-2.11 (m, 2H), 1.12 (s, 3H), 1.04 (s, 3H) ¹⁹F NMR (376 MHz, DMSO-d₆): −86.33 (s, 2F)

### Example 732: (*S)-4-[[6-(difluoromethoxy)-4-[2-[(2,6-dimethylpyrimidin-4-yl)amino]pyrazolo[1,5-a]pyridin-5-yl]-3-pyridyl]oxymethyl]-5,5-dimethyl-pyrrolidin-2-one

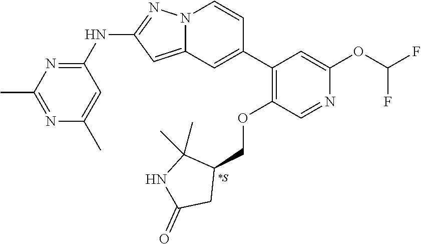 [SMILES: Cc1cc(Nc2cc3cc(-c4cc(OC(F)F)ncc4OC[C@H]4CC(=O)NC4(C)C)ccn3n2)nc(C)n1]

The title compound (21.2 mg, 25%) was prepared as described in Example 738. It was second to eluting peak. MS (ESI): mass calcd. for C₂₆H₂₇F₂N₇O₃, 523.2. m/z found, 524.6 [M+H]⁺. ¹H NMR (400 MHz, DMSO-d₆): δ 10.22 (s, 1H), 8.61 (d, J=6.8 Hz, 1H), 8.17 (s, 1H), 7.85-7.46 (m, 3H), 7.22 (s, 1H), 7.06-7.01 (m, 1H), 7.00 (dd, J=2.0, 7.2 Hz, 1H), 6.88 (br. s., 1H), 4.27-4.20 (m, 1H), 4.17-4.10 (m, 1H), 2.46 (s, 3H), 2.31 (s, 3H), 2.28-2.12 (m, 2H), 1.12 (s, 3H), 1.04 (s, 3H) ¹⁹F NMR (376 MHz, DMSO-d₆): −86.33 (s, 2F)

### Example 733: 5-[2-(difluoromethoxy)-5-(2-methyl-2-methylsulfonyl-propoxy)-4-pyridyl]-N-(2,6-dimethylpyrimidin-4-yl)pyrazolo[1,5-a]pyridin-2-amine

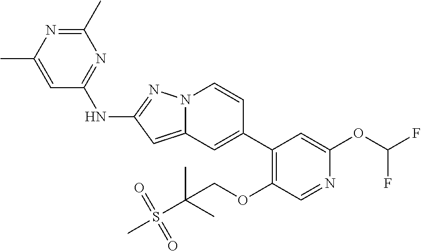 [SMILES: Cc1cc(Nc2cc3cc(-c4cc(OC(F)F)ncc4OCC(C)(C)S(C)(=O)=O)ccn3n2)nc(C)n1]

The title compound (38.8 mg, 24%) was prepared as described in Example 502 Steps A-B, except 2-methyl-2-(methylsulfonyl)propan-1-ol was used. MS (ESI): mass calcd. for C₂₄H₂₆F₂N₆O₄S, 532.2. m/z found, 533.3 [M+H]⁺. ¹H NMR (400 MHz, CD₃CN): δ 8.41 (d, J=7.2 Hz, 1H), 8.20 (s, 1H), 8.01 (s, 1H), 7.68 (d, J=0.8 Hz, 1H), 7.46 (t, J=73.6 Hz, 1H), 7.06 (s, 1H), 7.00 (s, 1H), 6.94 (dd, J=2.0, 7.2 Hz, 1H), 6.88 (s, 1H), 4.26 (s, 2H), 2.61 (s, 3H), 2.49 (s, 3H), 2.34 (s, 3H), 1.39 (s, 6H) ¹⁹F NMR (376 MHz, CD₃CN): −88.66 (s, 2F)

### Example 734: (*S)-4-[[6-(difluoromethoxy)-4-[2-[(2,6-dimethylpyrimidin-4-yl)amino]pyrazolo[1,5-a]pyridin-5-yl]-3-pyridyl]oxymethyl]-1,5,5-trimethyl-pyrrolidin-2-one

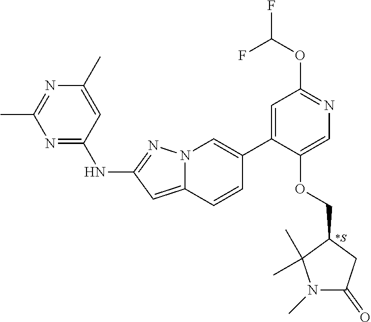 [SMILES: Cc1cc(Nc2cc3ccc(-c4cc(OC(F)F)ncc4OC[C@H]4CC(=O)N(C)C4(C)C)cn3n2)nc(C)n1]

Step A: ethyl 1,2,2-trimethyl-5-oxopyrrolidine-3-carboxylate. NaH (389 mg, 60% in mineral oil, 16.2 mmol) was added to a stirred mixture consisting of ethyl 2,2-dimethyl-5-oxopyrrolidine-3-carboxylate (1.5 g, 8.1 mmol) and DMF (20 mL) at 0° C. Then Mel (0.76 mL, 12 mmol) was added to the mixture at 0° C. and stirred at room temperature for 12 h. The reaction mixture was added to sat. aq. NH₄Cl (30 mL) and extracted with EtOAc (30 mL×3). The combined organic extracts were washed with water (50 mL×2) and brine (50 mL×3), dried over anhydrous Na₂SO₄, filtered, and concentrated to dryness under reduced pressure to give the product (1.4 g, 87%) as a yellow oil.

Step B: 2-methyl-2-(methylsulfonyl)propan-1-ol. The title compound (870 mg, 80%) was prepared as described in Example 365 Step A, except ethyl 1,2,2-trimethyl-5-oxopyrrolidine-3-carboxylate was used.

Step C: (1-cyanocyclopropyl)methyl 4-methylbenzenesulfonate. The title compound was prepared as described in Example 191 Step D.

Step D: (*S)-4-[[6-(difluoromethoxy)-4-[2-[(2,6-dimethylpyrimidin-4-yl)amino]pyrazolo[1,5-a]pyridin-5-yl]-3-pyridyl]oxymethyl]-1,5,5-trimethyl-pyrrolidin-2-one. The title compound (1.9 mg, 17%) was prepared as described in Example 329 except (1-cyanocyclopropyl)methyl 4-methylbenzenesulfonate was used in Step A. The resulting mixture was purified by SFC-13 over DAICEL CHIRALPAK AD 250 mm×30 mm, 10 μm (eluent: 40% to 40% (v/v) supercritical 0.1% NH₃H₂O MeOH to afford enantiomerically pure material. The title compound was second to elute. MS (ESI): mass calcd. for C₂₇H₂₉F₂N₇O₃, 537.2. m/z found, 538.2 [M+H]⁺. ¹H NMR (400 MHz, DMSO-d₆): δ 10.23 (s, 1H), 8.61 (d, J=6.8 Hz, 1H), 8.17 (s, 1H), 7.86-7.47 (m, 2H), 7.23 (s, 1H), 7.05-6.94 (m, 2H), 6.88 (br. s., 1H), 4.32-4.24 (m, 1H), 4.21-4.13 (m, 1H), 2.54 (s, 3H), 2.46 (s, 3H), 2.45-2.40 (m, 1H), 2.35-2.28 (m, 4H), 2.24-2.15 (m, 1H), 1.14 (s, 3H), 1.01 (s, 3H) ¹⁹F NMR (376 MHz, DMSO-d₆): −86.36 (s, 2F)

### Example 735: 3-[[6-(difluoromethoxy)-4-[2-[(2,6-dimethylpyrimidin-4-yl)amino]pyrazolo[1,5-a]pyridin-5-yl]-3-pyridyl]oxy]-N,N,2,2-tetramethyl-propanamide

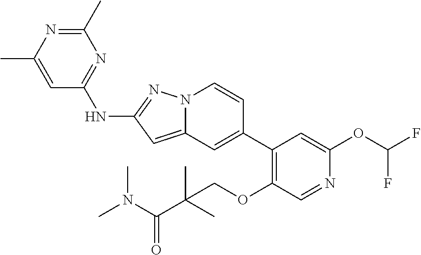 [SMILES: Cc1cc(Nc2cc3cc(-c4cc(OC(F)F)ncc4OCC(C)(C)C(=O)N(C)C)ccn3n2)nc(C)n1]

The title compound (74 mg, 34%) was prepared as described in Example 734, except dimethylamine hydrochloride was used in Step D. MS (ESI): mass calcd. for C₂₆H₂₉F₂N703, 525.2. m/z found, 526.3 [M+H]⁺. ¹H NMR (400 MHz, DMSO-d₆): δ 10.24 (s, 1H), 8.57 (d, J=7.2 Hz, 1H), 8.10 (s, 1H), 7.85 (d, J=0.8 Hz, 1H), 7.66 (t, J=1.2 Hz, 1H), 7.23 (s, 1H), 7.06-7.01 (m, 1H), 6.99 (dd, J=2.0, 7.2 Hz, 1H), 6.89 (s, 1H), 4.19 (s, 2H), 2.95 (s, 6H), 2.46 (s, 3H), 2.31 (s, 3H), 1.26 (s, 6H) ¹⁹F NMR (376 MHz, DMSO-d₆-86.32 (s, 2F)

### Example 736: 3-[[6-(difluoromethoxy)-4-[2-[(2,6-dimethylpyrimidin-4-yl)amino]imidazo[1,2-a]pyrazin-6-yl]-3-pyridyl]oxy]-2,2-dimethyl-propanenitrile

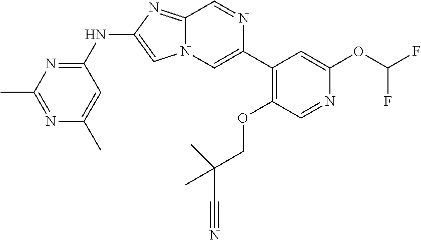 [SMILES: Cc1cc(Nc2cn3cc(-c4cc(OC(F)F)ncc4OCC(C)(C)C#N)ncc3n2)nc(C)n1]

Step A: (5-(2-cyano-2-methylpropoxy)-2-(difluoromethoxy)pyridin-4-yl)boronic acid. To a round bottom flask, i-PrMgCl·LiCl (8.36 mL, 10.9 mmol, 1.3 M in THF) was added drop-wise over 15 min to a −65° C. (dry ice/EtOH) solution consisting of 3-((6-(difluoromethoxy)-4-iodopyridin-3-yl)oxy)-2,2-dimethylpropanenitrile (1.00 g, 2.72 mmol) and THF (10 mL) and stirred at −65° C. for 30 min. Then 2-isopropoxy-4,4,5,5-tetramethyl-1,3,2-dioxaborolane (3.54 g, 19.0 mmol) was added drop-wise over 5 min to the reaction mixture under N₂ at −65° C. The mixture was warmed to room temperature and stirred overnight. The reaction mixture was poured into aq. Sat. NH₄Cl (50 mL) and extracting with EtOAc (50 mL×3). The combined organic extracts were concentrated to dryness under reduced pressure to give the crude product, which was purified by preparative HPLC using a Phenomenex DE Boston Uni C18 150×40 mm×5 um column (eluent: 30% to 60% (v/v) water (0.225% FA)-ACN) to afford pure product. The product was suspended in water (10 mL), the mixture frozen using dry ice/ethanol, and then lyophilized to dryness to afford the title compound (160 mg, 21%) as a yellow solid.

Step B: 3-[[6-(difluoromethoxy)-4-[2-[(2,6-dimethylpyrimidin-4-yl)amino]imidazo[1,2-a]pyrazin-6-yl]-3-pyridyl]oxy]-2,2-dimethyl-propanenitrile. The title compound was prepared as described in Example 259 Steps B-C, except (5-(2-cyano-2-methylpropoxy)-2-(difluoromethoxy)pyridin-4-yl)boronic acid was used and Brettphos-Pd-G3 was used as the catalyst in Step D (36.8 mg, 30%). MS (ESI): mass calcd. for C₂₃H₂₂F₂N₈O₂, 480.2. m/z found, 481.1 [M+H]⁺. ¹H NMR (400 MHz, DMSO-d₆): δ 10.52 (s, 1H), 9.44 (d, J=1.2 Hz, 1H), 9.01 (s, 1H), 8.51 (s, 1H), 8.20 (s, 1H), 7.86-7.45 (m, 2H), 6.73 (s, 1H), 4.35 (s, 2H), 2.48 (s, 3H), 2.30 (s, 3H), 1.46 (s, 6H) ¹⁹F NMR (376 MHz, DMSO-d₆): −86.23 (s, 2F)

### Example 737: 3-((4-(2-((1-cyclopropyl-5-methyl-6-oxo-1,6-dihydropyridazin-3-yl)amino)pyrazolo[1,5-a]pyridin-5-yl)-6-(2,2-difluoroethoxy)pyridin-3-yl)oxy)-2,2-dimethylpropanenitrile

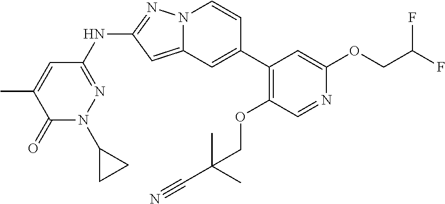 [SMILES: Cc1cc(Nc2cc3cc(-c4cc(OCC(F)F)ncc4OCC(C)(C)C#N)ccn3n2)nn(C2CC2)c1=O]

The title compound (43.2 mg, 20%) was prepared as described in Example 695, except Brettphos-Pd-G3 was used as the catalyst. MS (ESI): mass calcd. for C₂₇H₂₇F₂N₇O₃, 535.2. m/z found, 536.5 [M+H]⁺. ¹H NMR (400 MHz, DMSO-d₆): δ 9.82 (s, 1H), 8.54 (d, J=7.2 Hz, 1H), 8.05 (s, 1H), 7.91 (d, J=1.2 Hz, 1H), 7.23 (d, J=1.6 Hz, 1H), 7.10 (s, 1H), 7.05 (dd, J=2.0, 7.2 Hz, 1H), 6.86 (s, 1H), 6.57-6.25 (m, 1H), 6.05-5.94 (m, 1H), 5.30-5.21 (m, 2H), 4.65-4.52 (m, 4H), 4.14 (s, 2H), 2.10 (d, J=1.2 Hz, 3H), 1.35 (s, 6H) ¹⁹F NMR (376 MHz, DMSO-d₆): −125.53 (s, 2F)

### Example 738: (*R)-4-[[6-(difluoromethoxy)-4-[2-[(2,6-dimethylpyrimidin-4-yl)amino]pyrazolo[1,5-a]pyridin-5-yl]-3-pyridyl]oxymethyl]-5,5-dimethyl-pyrrolidin-2-one

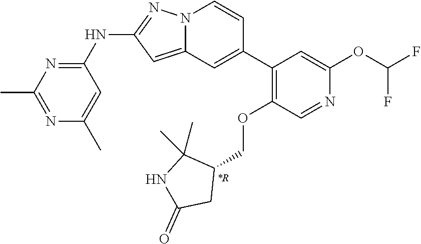 [SMILES: Cc1cc(Nc2cc3cc(-c4cc(OC(F)F)ncc4OC[C@@H]4CC(=O)NC4(C)C)ccn3n2)nc(C)n1]

The title compound (85 mg, 20%) was prepared as described in Example 501 Steps A-B, except 4-(hydroxymethyl)-5,5-dimethylpyrrolidin-2-one was used. The enantiomeric mixture was purified by SFC-16 over DAICEL CHIRALCEL OJ-H 250 mm×30 mm×5 μm (eluent: 45% to 45% (v/v) MeOH with 0.1% NH₃·H₂O in supercritical CO₂). The title compound was first to elute. MS (ESI): mass calcd. for C₂₆H₂₇F₂N₇O₃, 523.2. m/z found, 524.6 [M+H]⁺. ¹H NMR (400 MHz, DMSO-d₆): δ 10.22 (s, 1H), 8.61 (d, J=7.2 Hz, 1H), 8.17 (s, 1H), 7.85-7.46 (m, 3H), 7.22 (s, 1H), 7.07-7.01 (m, 1H), 7.00 (dd, J=1.6, 7.2 Hz, 1H), 6.88 (br. s., 1H), 4.27-4.20 (m, 1H), 4.17-4.09 (m, 1H), 2.46 (s, 3H), 2.31 (s, 3H), 2.28-2.11 (m, 2H), 1.12 (s, 3H), 1.04 (s, 3H) ¹⁹F NMR (376 MHz, DMSO-d₆): −86.33 (s, 2F)

### Example 739: (*S)-4-[[6-(difluoromethoxy)-4-[2-[(2,6-dimethylpyrimidin-4-yl)amino]pyrazolo[1,5-a]pyridin-5-yl]-3-pyridyl]oxymethyl]-5,5-dimethyl-pyrrolidin-2-one

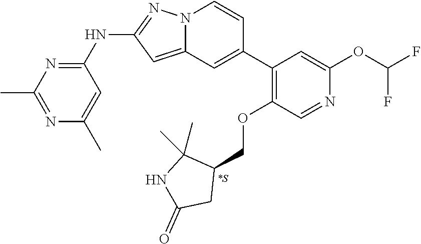 [SMILES: Cc1cc(Nc2cc3cc(-c4cc(OC(F)F)ncc4OC[C@H]4CC(=O)NC4(C)C)ccn3n2)nc(C)n1]

The title compound (21.2 mg, 25%) was prepared as described in Example 738. It was second to eluting peak. MS (ESI): mass calcd. for C₂₆H₂₇F₂N₇O₃, 523.2. m/z found, 524.6 [M+H]⁺. ¹H NMR (400 MHz, DMSO-d₆): δ 10.22 (s, 1H), 8.61 (d, J=6.8 Hz, 1H), 8.17 (s, 1H), 7.85-7.46 (m, 3H), 7.22 (s, 1H), 7.06-7.01 (m, 1H), 7.00 (dd, J=2.0, 7.2 Hz, 1H), 6.88 (br. s., 1H), 4.27-4.20 (m, 1H), 4.17-4.10 (m, 1H), 2.46 (s, 3H), 2.31 (s, 3H), 2.28-2.12 (m, 2H), 1.12 (s, 3H), 1.04 (s, 3H) ¹⁹F NMR (376 MHz, DMSO-d₆): −86.33 (s, 2F)

### Example 740: 5-[2-(difluoromethoxy)-5-(2-methyl-2-methylsulfonyl-propoxy)-4-pyridyl]-N-(2,6-dimethylpyrimidin-4-yl)pyrazolo[1,5-a]pyridin-2-amine

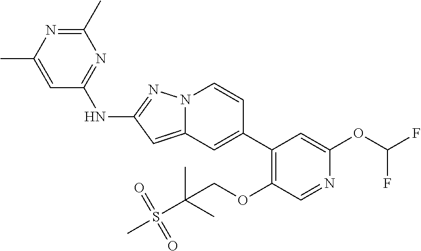 [SMILES: Cc1cc(Nc2cc3cc(-c4cc(OC(F)F)ncc4OCC(C)(C)S(C)(=O)=O)ccn3n2)nc(C)n1]

The title compound (38.8 mg, 24%) was prepared as described in Example 501 Steps A-B, except 2-methyl-2-(methylsulfonyl)propan-1-ol was used. MS (ESI): mass calcd. for C₂₄H₂₆F₂N₆O₄S, 532.2. m/z found, 533.3 [M+H]⁺. ¹H NMR (400 MHz, CD₃CN): δ 8.41 (d, J=7.2 Hz, 1H), 8.20 (s, 1H), 8.01 (s, 1H), 7.68 (d, J=0.8 Hz, 1H), 7.46 (t, J=73.6 Hz, 1H), 7.06 (s, 1H), 7.00 (s, 1H), 6.94 (dd, J=2.0, 7.2 Hz, 1H), 6.88 (s, 1H), 4.26 (s, 2H), 2.61 (s, 3H), 2.49 (s, 3H), 2.34 (s, 3H), 1.39 (s, 6H) ¹⁹F NMR (376 MHz, CD₃CN): −88.66 (s, 2F)

### Example 741: (*S)-4-[[6-(difluoromethoxy)-4-[2-[(2,6-dimethylpyrimidin-4-yl)amino]pyrazolo[1,5-a]pyridin-5-yl]-3-pyridyl]oxymethyl]-1,5,5-trimethyl-pyrrolidin-2-one

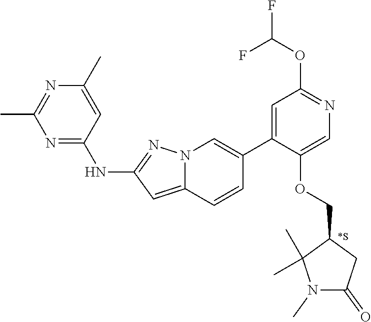 [SMILES: Cc1cc(Nc2cc3ccc(-c4cc(OC(F)F)ncc4OC[C@H]4CC(=O)N(C)C4(C)C)cn3n2)nc(C)n1]

Step A: ethyl 1,2,2-trimethyl-5-oxopyrrolidine-3-carboxylate. NaH (389 mg, 60% in mineral oil, 16.2 mmol) was added to a stirred mixture consisting of ethyl 2,2-dimethyl-5-oxopyrrolidine-3-carboxylate (1.5 g, 8.1 mmol) and DMF (20 mL) at 0° C. Then Mel (0.76 mL, 12 mmol) was added to the mixture at 0° C. and stirred at room temperature for 12 h. The reaction mixture was added to sat. aq. NH₄Cl (30 mL) and extracted with EtOAc (30 mL×3). The combined organic extracts were washed with water (50 mL×2) and brine (50 mL×3), dried over anhydrous Na₂SO₄, filtered, and concentrated to dryness under reduced pressure to give the product (1.4 g, 87%) as a yellow oil.

Step B: 2-methyl-2-(methylsulfonyl)propan-1-ol. The title compound (870 mg, 80%) was prepared as described in Example 364 Step A, except ethyl 1,2,2-trimethyl-5-oxopyrrolidine-3-carboxylate was used.

Step C: (1-cyanocyclopropyl)methyl 4-methylbenzenesulfonate. The title compound was prepared as described in Example 191 Step D.

Step D: (*S)-4-[[6-(difluoromethoxy)-4-[2-[(2,6-dimethylpyrimidin-4-yl)amino]pyrazolo[1,5-a]pyridin-5-yl]-3-pyridyl]oxymethyl]-1,5,5-trimethyl-pyrrolidin-2-one. The title compound (1.9 mg, 17%) was prepared as described in Example 329 except (1-cyanocyclopropyl)methyl 4-methylbenzenesulfonate was used in Step A. The resulting mixture was purified by SFC-13 over DAICEL CHIRALPAK AD 250 mm×30 mm, 10 μm (eluent: 40% to 40% (v/v) supercritical 0.1% NH₃H₂O MeOH to afford enantiomerically pure material. The title compound was second to elute. MS (ESI): mass calcd. for C₂₇H₂₉F₂N₇O₃, 537.2. m/z found, 538.2 [M+H]⁺. ¹H NMR (400 MHz, DMSO-d₆): δ 10.23 (s, 1H), 8.61 (d, J=6.8 Hz, 1H), 8.17 (s, 1H), 7.86-7.47 (m, 2H), 7.23 (s, 1H), 7.05-6.94 (m, 2H), 6.88 (br. s., 1H), 4.32-4.24 (m, 1H), 4.21-4.13 (m, 1H), 2.54 (s, 3H), 2.46 (s, 3H), 2.45-2.40 (m, 1H), 2.35-2.28 (m, 4H), 2.24-2.15 (m, 1H), 1.14 (s, 3H), 1.01 (s, 3H) ¹⁹F NMR (376 MHz, DMSO-d₆): −86.36 (s, 2F)

### Additional Compounds

Additional compounds were prepared and listed in the Table 1 below:

The following exemplified compounds were prepared using methods analogous to those cited in the synthesis method column.

TABLE 1
Additional Compounds

| | | | Syn- | |
|---|---|---|---|---|
| Ex- | | | thesis | Compound IUPAC |
| ample | Structure | NMR | Method | name |
| 742 | 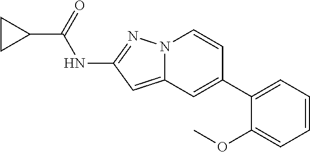 [SMILES: COc1ccccc1-c1ccn2nc(NC(=O)C3CC3)cc2c1] | 1H NMR (400 MHz, DMSO-d6): 11.03 (s, 1H), 8.49 (d, J = 6.8 Hz, 1H), 7.62 (d, J = 1.2 Hz, 1H), 7.47- 7.33 (m, 2H), 7.15 (d, J = 8.0 Hz, 1H), 7.09-7.02 (m, 1H), 6.94 (dd, J = 1.6, 7.2 Hz, 1H), 6.83 (s, 1H), 3.81 (s, 3H), 1.99-1.85 (m, 1H), 0.90-0.71 (m, 4H) | Int. 5 | N-[5-(2- methoxyphenyl)pyrazolo[1,5- a]pyridin-2- yl]cyclopropanecarboxamide |
| 743 | 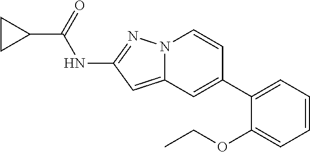 [SMILES: CCOc1ccccc1-c1ccn2nc(NC(=O)C3CC3)cc2c1] | 1H NMR (400 MHz, DMSO-d6): 11.04 (s, 1H), 8.49 (d, J = 7.2 Hz, 1H), 7.67-7.63 (m, 1H), 7.41 (dd, J = 1.6, 7.6 Hz, 1H), 7.39-7.33 (m, 1H), 7.12 (d, J = 8.0 Hz, 1H), 7.07- 7.02 (m, 1H), 6.99 (dd, J = 2.0, 7.2 Hz, 1H), 6.83 (s, 1H), 4.08 (q, J = 7.2 Hz, 2H), 1.97-1.88 (m, 1H), 1.30 (t, J = 6.8 Hz, 3H), 0.86-0.77 (m, 4H) | Int. 5 | N-[5-(2- ethoxyphenyl)pyrazolo[1,5- a]pyridin-2- yl]cyclopropanecarboxamide |
| 744 | 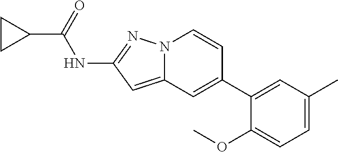 [SMILES: COc1ccc(C)cc1-c1ccn2nc(NC(=O)C3CC3)cc2c1] | 1H NMR (400 MHz, DMSO-d6): 11.03 (s, 1H), 8.48 (d, J = 7.2 Hz, 1H), 7.61 (d, J = 1.6 Hz, 1H), 7.23- 7.16 (m, 2H), 7.03 (d, J = 8.4 Hz, 1H), 6.93 (dd, J = 2.0, 7.2 Hz, 1H), 6.82 (s, 1H), 3.77 (s, 3H), 2.30 (s, 3H), 1.97-1.88 (m, 1H), 0.85-0.77 (m, 4H) | Int. 5 | N-[5-(2-methoxy-5- methyl- phenyl)pyrazolo[1,5- a]pyridin-2- yl]cyclopropanecarboxamide |
| 745 | 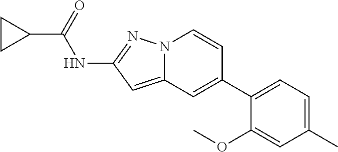 [SMILES: COc1cc(C)ccc1-c1ccn2nc(NC(=O)C3CC3)cc2c1] | 1H NMR (400 MHz, DMSO-d6): 11.01 (s, 1H), 8.47 (d, J = 7.2 Hz, 1H), 7.59 (d, J = 1.2 Hz, 1H), 7.28 (d, J = 7.6 Hz, 1H), 6.97 (s, 1H), 6.92 (dd, J = 2.0, 7.2 Hz, 1H), 6.87 (d, J = 7.6 Hz, 1H), 6.81 (s, 1H), 3.79 (s, 3H), 2.36 (s, 3H), 1.97-1.87 (m, 1H), 0.88-0.76 (m, 4H) | Int. 5 | N-[5-(2-methoxy-4- methyl- phenyl)pyrazolo[1,5- a]pyridin-2- yl]cyclopropanecarboxamide |
| 746 | 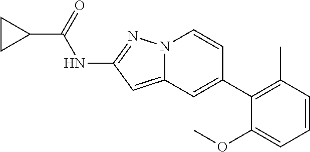 [SMILES: COc1cccc(C)c1-c1ccn2nc(NC(=O)C3CC3)cc2c1] | 1H NMR (400 MHz, DMSO-d6): 11.03 (s, 1H), 8.50 (d, J = 7.2 Hz, 1H), 7.34 (s, 1H), 7.31-7.24 (m, 1H), 6.96-6.90 (m, 2H), 6.80 (s, 1H), 6.58 (dd, J = 1.6, 7.2 Hz, 1H), 3.67 (s, 3H), 2.09 (s, 3H), 1.98-1.88 (m, 1H), 0.88-0.87 (m, 4H) | Int. 5 | N-[5-(2-methoxy-6- methyl- phenyl)pyrazolo[1,5- a]pyridin-2- yl]cyclopropanecarboxamide |
| 747 | 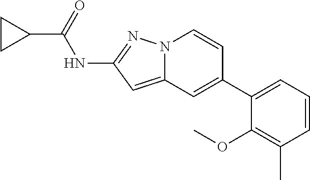 [SMILES: COc1c(C)cccc1-c1ccn2nc(NC(=O)C3CC3)cc2c1] | 1H NMR (400 MHz, DMSO-d6): 11.06 (s, 1H), 8.54 (d, J = 7.2 Hz, 1H), 7.69 (d, J = 1.2 Hz, 1H), 7.32- 7.23 (m, 2H), 7.17-7.10 (m, 1H), 7.00 (dd, J = 1.6, 7.2 Hz, 1H), 6.86 (s, 1H), 3.41 (s, 3H), 2.30 (s, 3H), 1.99-1.87 (m, 1H), 0.89-0.73 (m, 4H) | Int. 5 | N-[5-(2-methoxy-3- methyl- phenyl)pyrazolo[1,5- a]pyridin-2- yl]cyclopropanecarboxamide |
| 748 | 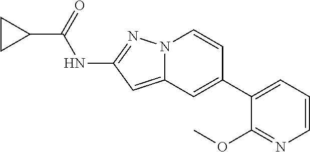 [SMILES: COc1ncccc1-c1ccn2nc(NC(=O)C3CC3)cc2c1] | 1H NMR (400 MHz, DMSO-d6): 11.07 (s, 1H), 8.54 (d, J = 7.2 Hz, 1H), 8.22 (dd, J = 1.6, 4.8 Hz, 1H), 7.88 (dd, J = 1.6, 7.2 Hz, 1H), 7.76 (d, J = 1.2 Hz, 1H), 7.13 (dd, J = 4.8, 7.2 Hz, 1H), 7.02 (dd, J = 2.0, 7.2 Hz, 1H), 6.86 (s, 1H), 3.93 (s, 3H), 1.98-1.89 (m, 1H), 0.87-0.77 (m, 4H) | Int. 5 | N-[5-(2-methoxy-3- pyridyl)pyrazolo[1,5- a]pyridin-2- yl]cyclopropanecarboxamide |
| 749 | 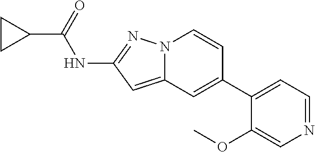 [SMILES: COc1cnccc1-c1ccn2nc(NC(=O)C3CC3)cc2c1] | 1H NMR (400 MHz, DMSO-d6): 11.08 (s, 1H), 8.71-8.51 (m, 2H), 8.42 (d, J = 4.8 Hz, 1H), 7.88 (s, 1H), 7.67 (d, J = 5.2 Hz, 1H), 7.05 (dd, J = 2.0, 7.6 Hz, 1H), 6.93 (s, 1H), 3.98 (s, 3H), 2.01-1.86 (m, 1H), 0.90-0.73 (m, 4H) | Int. 5 | N-[5-(3-methoxy-4- pyridyl)pyrazolo[1,5- a]pyridin-2- yl]cyclopropanecarboxamide |
| 750 | 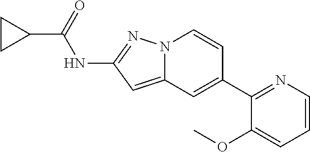 [SMILES: COc1cccnc1-c1ccn2nc(NC(=O)C3CC3)cc2c1] | 1H NMR (400 MHz, DMSO-d6): 11.07 (s, 1H), 8.54 (d, J = 7.2 Hz, 1H), 8.30 (dd, J = 1.2, 4.4 Hz, 1H), 8.20 (d, J = 1.2 Hz, 1H), 7.63 (dd, J = 0.8, 8.4 Hz, 1H), 7.47-7.38 (m, 2H), 6.90 (s, 1H), 3.93 (s, 3H), 2.00-1.87 (m, 1H), 0.90-0.74 (m, 4H) | Int. 5 | N-[5-(3-methoxy-2- pyridyl)pyrazolo[1,5- a]pyridin-2- yl]cyclopropanecarboxamide |
| 751 | 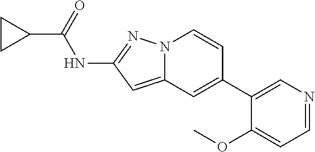 [SMILES: COc1ccncc1-c1ccn2nc(NC(=O)C3CC3)cc2c1] | 1H NMR (400 MHz, DMSO-d6): 11.07 (s, 1H), 8.54 (d, J = 7.2 Hz, 1H), 8.52-8.45 (m, 2H), 7.71 (d, J = 1.2 Hz, 1H), 7.21 (d, J = 5.6 Hz, 1H), 6.97 (dd, J = 1.6, 6.8 Hz, 1H), 6.86 (s, 1H), 3.90 (s, 3H), 2.01-1.85 (m, 1H), 0.91-0.72 (m, 4H) | Int. 5 | N-[5-(4-methoxy-3- pyridyl)pyrazolo[1,5- a]pyridin-2- yl]cyclopropanecarboxamide |
| 752 | 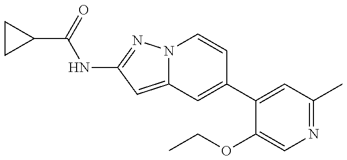 [SMILES: CCOc1cnc(C)cc1-c1ccn2nc(NC(=O)C3CC3)cc2c1] | 1H NMR (400 MHz, DMSO-d6): 11.10 (s, 1H), 8.56 (d, J = 6.8 Hz, 1H), 8.32 (s, 1H), 7.81 (s, 1H), 7.34 (s, 1H), 7.09-7.01 (m, 1H), 6.89 (s, 1H), 4.16 (q, J = 6.8 Hz, 2H), 2.46 (s, 3H), 2.01-1.87 (m, 1H), 1.31 (t, J = 7.2 Hz, 3H), 0.89-0.72 (m, 4H) | Int. 5 | N-[5-(5-ethoxy-2-methyl- 4-pyridyl)pyrazolo[1,5- a]pyridin-2- yl]cyclopropanecarboxamide |
| 753 | 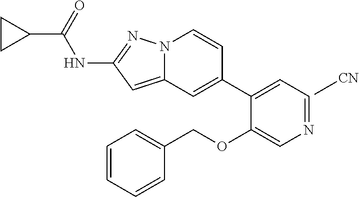 | 1H NMR (400 MHz, DMSO-d6): 11.12 (s, 1H), 8.77 (s, 1H), 8.60 (d, J = 7.2 Hz, 1H), 8.20 (s, 1H), 7.94 (s, 1H), 7.50-7.44 (m, 2H), 7.42-7.32 (m, 3H), 7.12 (dd, J = 1.2, 7.2 Hz, 1H), 6.89 (s, 1H), 5.46 (s, 2H), 1.98-1.88 (m, 1H), 0.87-0.78 (m, 4H) | Example 1 | N-[5-(5-benzyloxy-2- cyano-4- pyridyl)pyrazolo[1,5- a]pyridin-2- yl]cyclopropanecarboxamide |
| 754 | 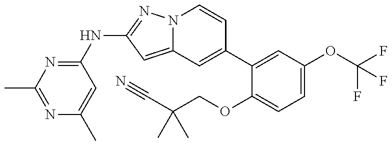 [SMILES: Cc1cc(Nc2cc3cc(-c4cc(OC(F)(F)F)ccc4OCC(C)(C)C#N)ccn3n2)nc(C)n1] | 1H NMR (400 MHz, DMSO-d6) d 10.18 (s, 1H), 8.59-8.51 (m, 1H), 7.79-7.74 (m, 1H), 7.51-7.48 (m, 1H), 7.46-7.39 (m, 1H), 7.27 (d, J = 9.1 Hz, 1H), 7.05 (s, 1H), 7.00 (dd, J = 7.2, 2.0 Hz, 1H), 6.81 (s, 1H), 4.14 (s, 2H), 2.45 (s, 3H), 2.30 (s, 3H), 1.35 (s, 6H). | Example 203 | 3-[2-[2-[(2,6- dimethylpyrimidin-4- yl)amino]pyrazolo[1,5- a]pyridin-5-yl]-4- (trifluoromethoxy)phenoxy]- 2,2-dimethyl- propanenitrile |
| 755 | 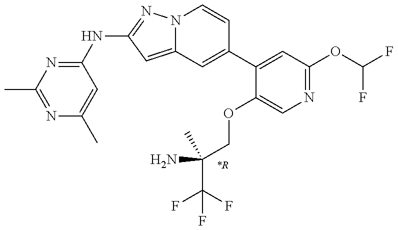 | 1H NMR (400 MHz, DMSO-d6): 10.24 (s, 1H), 8.62 (d, J = 7.2 Hz, 1H), 8.15 (s, 1H), 7.87-7.86 (m, 1H), 7.68 (t, J = 73.2 Hz, 1H), 7.25 (s, 1H), 7.05-7.00 (m, 2H), 6.86 (s, 1H), 4.18-4.07 (m, 2H), 2.46 (s, 3H), 2.31 (s, 3H), 2.14 (s, 2H), 1.22 (s, 3H) | Example 267 | (*R)-5-[5-(2-amino-3,3,3- trifluoro-2-methyl- propoxy)-2- (difluoromethoxy)-4- pyridyl]-N-(2,6- dimethylpyrimidin-4- yl)pyrazolo[1,5-a]pyridin- 2-amine |
| 756 | 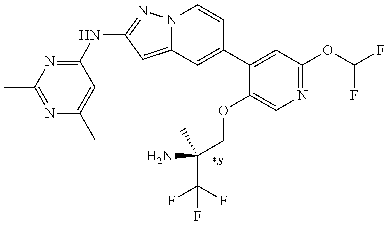 | 1H NMR (400 MHz, DMSO-d6): 10.25 (s, 1H), 8.62 (d, J = 7.2 Hz, 1H), 8.16 (s, 1H), 7.88-7.48 (m, 2H), 7.25 (s, 1H), 7.07-7.00 (m, 2H), 6.87 (s, 1H), 4.23-4.03 (m, 2H), 2.46 (s, 3H), 2.31 (s, 3H), 2.15 (s, 2H, 1.22 (s, 3H) | Example 267 | (*S)-5-[5-(2-amino-3,3,3- trifluoro-2-methyl- propoxy)-2- (difluoromethoxy)-4- pyridyl]-N-(2,6- dimethylpyrimidin-4- yl)pyrazolo[1,5-a]pyridin- 2-amine |

Note: Int. 5 = Intermediate 5

### BIOLOGICAL TESTING EXAMPLES

### Test Example 1: SIK2 Enzymatic Assay Version 1

All steps were performed at room temperature. Compounds were pre-plated onto an assay plate using an Echo acoustic dispenser (Labcyte, E550 or E555) at 20 nL per well. Enzyme prepared in Assay Buffer at 0.6 nM (2× final concentration) was added at 2 μL per well using the Multidrop Combi (Thermo Fisher—used for all additions in this assay) to the assay plate, spun for 1 min at 800-850 rpm (Allegra 6KR Beckman Coulter centrifuge), and incubated with compound for 30 minutes. Assay Buffer containing 160 μM ATP and 140 μM HDAC5tide (2× of final concentration for each substrate) was added at 2 μL per well to the assay plate and then spun as above and incubated for 4 hours.

One column of wells was dedicated to a no-peptide negative control (containing only ATP). ADPGlo Reagent (prepared and aliquoted as per manufacturer instructions) was then added at 2 μL per well, spun and incubated for 40 min. ADPGlo Detection (prepared and aliquoted as per manufacturer instructions) was added at 2 μL per well to the assay plate, which was spun and incubated for at least 30 min. The plate was then read on the Pherastar (BMG Labtech) using a luminescence read. Gain was set by the 0.1 mM concentration of ADP wells in the ADP/ATP standard curve plate generated as per manufacturer instructions.

The results are shown in Table 2 below.

### Test Example 2: SIK2 Enzymatic Assay High Version 2 (High ATP)

All steps were performed at room temperature. Compounds were pre-plated onto an assay plate using an Echo acoustic dispenser (Labcyte, E550 or E555) at 80 nL per well. Enzyme prepared in assay buffer at 6 nM (2× final concentration) was added at 4 μL per well using the Multidrop Combi (Thermo Fisher—used for all additions in this assay) to the assay plate, spun for 1 min at 1000 rpm (Allegra 6KR Beckman Coulter centrifuge), and incubated with compound for 30 minutes. Assay buffer containing 6 mM ATP and 400 μM HDAC5tide (2× of final concentration for each substrate) was added at 4 μL per well to the assay plate and then spun as above and incubated for 2 hours. Note that typically one column of wells was dedicated to a no-enzyme negative control (containing ATP and peptide) and another dedicated to DMSO only (maximal enzyme activity). ADP-Glo Max Reagent (prepared and aliquoted as per manufacturer instructions) was then added at 8 μL per well, spun and incubated for 2 hours. ADP-Glo Max Detection (prepared and aliquoted as per manufacturer instructions) was added at 8 μL per well to the assay plate, which was spun and incubated for 1 hour. The plate is then read on the Pherastar (BMG Labtech) using a luminescence read. Gain is set between 2200-2500.

The results are shown in Table 2 below.

TABLE 2
SIK2 Enzymatic Assay Data

| | SIK2 ADP Glo IC₅₀ (μM) | SIK2 high ATP IC₅₀ (μM) |
|---|---|---|
| EXAMPLE Number | (Test Example 1) | (Test Example 2) |
| Example 1 | 0.00226099 | 0.0591017 |
| Example 2 | 0.0691035 | |
| Example 3 | 0.0183274 | |
| Example 4 | 0.00389853 | 0.0739946 |
| Example 5 | 0.00038054 | 0.00773926 |
| Example 6 | 0.00149314 | 0.0112461 |
| Example 7 | 0.00072477 | 0.0298882 |
| Example 8 | 0.000372821 | 0.00750584 |
| Example 9 | 0.15209 | |
| Example 10 | 0.204221 | 4.8708 |
| Example 11 | 0.00139862 | 0.0316301 |
| Example 12 | 0.0133168 | |
| Example 13 | 0.0208209 | 0.304649 |
| Example 14 | 0.0248886 | |
| Example 15 | 0.00270209 | 0.0398657 |
| Example 16 | 0.00496135 | 0.236265 |
| Example 17 | 0.000402253 | 0.00417445 |
| Example 18 | 0.00157906 | |
| Example 19 | 0.00078343 | 0.00837914 |
| Example 20 | 0.0012218 | 0.0101976 |
| Example 21 | 0.0213108 | |
| Example 22 | 0.00121227 | |
| Example 23 | 0.00209701 | 0.0163493 |
| Example 24 | 0.00594018 | 0.14471 |
| Example 25 | 0.00145479 | |
| Example 26 | 0.0122744 | |
| Example 27 | 0.00473587 | 0.0986961 |
| Example 28 | 0.00921721 | |
| Example 29 | 0.00575175 | 0.144944 |
| Example 30 | 0.000439643 | 0.00449987 |
| Example 31 | 0.00704855 | 0.111148 |
| Example 32 | 0.0033986 | 0.0813018 |
| Example 33 | 0.00416006 | 0.0931108 |
| Example 34 | 0.0226986 | |
| Example 35 | 0.0354323 | |
| Example 36 | 0.0032144 | 0.0200678 |
| Example 37 | 0.00724602 | 0.111096 |
| Example 38 | 0.0139605 | 0.30486 |
| Example 39 | 0.00151635 | 0.0359501 |
| Example 40 | 0.00470435 | 0.0875992 |
| Example 41 | 0.00243053 | 0.0584925 |
| Example 42 | 0.00169044 | 0.0454569 |
| Example 43 | 0.000937562 | 0.0108044 |
| Example 44 | 0.0012368 | 0.0259777 |
| Example 45 | 0.00116091 | 0.0233668 |
| Example 46 | 0.0140637 | 0.184757 |
| Example 47 | 0.0134896 | 0.154099 |
| Example 48 | 0.0109295 | 0.117788 |
| Example 49 | 0.000957194 | 0.0252232 |
| Example 50 | 0.00111045 | 0.0219079 |
| Example 51 | 0.0050898 | 0.115638 |
| Example 52 | 0.00205636 | 0.03182 |
| Example 53 | 0.00064121 | 0.0135394 |
| Example 54 | 0.00411623 | 0.0759976 |
| Example 55 | 0.00960285 | |
| Example 56 | 0.00360246 | 0.0691672 |
| Example 57 | 0.0102683 | 0.263937 |
| Example 58 | 0.0043132 | 0.099862 |
| Example 59 | 0.00470219 | 0.110688 |
| Example 60 | 0.00492493 | 0.0985826 |
| Example 61 | 0.0102565 | |
| Example 62 | >5.00034 | |
| Example 63 | 0.004255 | 0.0786321 |
| Example 64 | 0.000387258 | 0.0086258 |
| Example 65 | 0.00870963 | |
| Example 66 | 0.000561306 | 0.00849572 |
| Example 67 | 0.000630521 | 0.0086258 |
| Example 68 | 0.0006665 | 0.0240381 |
| Example 69 | 0.00280479 | 0.0383442 |
| Example 70 | 0.0208401 | 0.408696 |
| Example 71 | 0.0576899 | |
| Example 72 | 0.15028 | |
| Example 73 | 0.0846058 | |
| Example 74 | 0.00654485 | |
| Example 75 | 0.398015 | |
| Example 76 | 0.0255211 | |
| Example 77 | 0.241936 | |
| Example 78 | 0.0143153 | |
| Example 79 | 0.388687 | |
| Example 80 | 0.0703396 | |
| Example 81 | 0.0154454 | 0.277524 |
| Example 82 | 0.000268287 | 0.00506874 |
| Example 83 | 0.00299433 | 0.0567283 |
| Example 84 | 0.0139766 | |
| Example 85 | 0.00255741 | 0.0506991 |
| Example 86 | 0.000204127 | 0.00275613 |
| Example 87 | 0.117571 | |
| Example 88 | 0.0029792 | 0.0511329 |
| Example 89 | 0.000299157 | 0.00303599 |
| Example 90 | 0.0133721 | |
| Example 91 | 0.0687701 | |
| Example 92 | 0.000130377 | <0.000838301 |
| Example 93 | 0.035917 | 1.24566 |
| Example 94 | 0.00846838 | 0.167109 |
| Example 95 | 0.000435011 | 0.00258107 |
| Example 96 | 0.00091159 | 0.0160214 |
| Example 97 | 0.00608695 | 0.114472 |
| Example 98 | 0.000779471 | 0.0137784 |
| Example 99 | 0.00295325 | 0.0376184 |
| Example 100 | 0.00572927 | 0.105463 |
| Example 101 | 0.00262543 | 0.0218726 |
| Example 102 | 0.000413048 | <0.000838301 |
| Example 103 | 0.0036241 | 0.0344588 |
| Example 104 | 0.00587896 | 0.0310384 |
| Example 105 | 0.00951043 | 0.202908 |
| Example 106 | 0.00338143 | 0.0484507 |
| Example 107 | 0.0022009 | 0.0400959 |
| Example 108 | 0.00527352 | 0.203939 |
| Example 109 | 0.00421891 | 0.116386 |
| Example 110 | 0.000549667 | 0.00285102 |
| Example 111 | 0.00260616 | 0.047852 |
| Example 112 | 0.00356369 | 0.0814704 |
| Example 113 | 0.000393188 | 0.00227353 |
| Example 114 | 0.000337365 | 0.00658264 |
| Example 115 | 0.00949949 | 0.158891 |
| Example 116 | | <0.000846838 |
| Example 117 | | 0.00090365 |
| Example 118 | | <0.000846838 |
| Example 119 | | 0.00127321 |
| Example 120 | | <0.000846838 |
| Example 121 | | 0.00115611 |
| Example 122 | | 2.42605 |
| Example 123 | | 0.000939508 |
| Example 124 | | 0.00123709 |
| Example 125 | | <0.000846838 |
| Example 126 | | <0.000846838 |
| Example 127 | | <0.000846838 |
| Example 128 | | 0.0248828 |
| Example 129 | 0.000657354 | 0.00827752 |
| Example 130 | | 0.00428845 |
| Example 131 | | 0.0523842 |
| Example 132 | | 0.00951481 |
| Example 133 | | 0.00844694 |
| Example 134 | | 0.012841 |
| Example 135 | | 0.00986053 |
| Example 136 | 0.000207635 | <0.000838301 |
| Example 137 | 0.00333657 | 0.0700487 |
| Example 138 | 0.000854279 | 0.0129122 |
| Example 139 | 0.00330293 | 0.0728954 |
| Example 140 | 0.0194581 | |
| Example 141 | 0.0138357 | |
| Example 142 | 0.0448023 | |
| Example 143 | 0.000664049 | 0.0126212 |
| Example 144 | 0.00211592 | 0.0364418 |
| Example 145 | 0.000642244 | 0.0164021 |
| Example 146 | 0.0351803 | |
| Example 147 | 0.0124194 | |
| Example 148 | 0.00436315 | 0.0583042 |
| Example 149 | 0.0121787 | |
| Example 150 | 0.00643872 | 0.322181 |
| Example 151 | | 0.00377572 |
| Example 152 | 0.00378007 | 0.0701294 |
| Example 153 | 0.000688177 | 0.0136679 |
| Example 154 | 0.00116065 | 0.0239718 |
| Example 155 | 0.00108168 | 0.0156099 |
| Example 156 | 0.00368638 | 0.0787589 |
| Example 157 | | 0.0708435 |
| Example 158 | 0.00131826 | 0.0183105 |
| Example 159 | | 0.0107498 |
| Example 160 | | 0.0444222 |
| Example 161 | | <0.000846838 |
| Example 162 | | 0.0195614 |
| Example 163 | | 0.0079506 |
| Example 164 | | 0.218072 |
| Example 165 | | 0.00822812 |
| Example 166 | 0.00101321 | 0.0158526 |
| Example 167 | 0.000589929 | 0.00880441 |
| Example 168 | 0.000382472 | 0.00473042 |
| Example 169 | 0.00307043 | 0.0507224 |
| Example 170 | 0.000852512 | 0.0128529 |
| Example 171 | 0.000617305 | 0.01228 |
| Example 172 | | 0.00087579 |
| Example 173 | | <0.000846838 |
| Example 174 | | 0.108118 |
| Example 175 | 0.000442895 | 0.00844502 |
| Example 176 | 0.0019611 | 0.0555392 |
| Example 177 | 0.0013712 | 0.0293224 |
| Example 178 | 0.00060534 | 0.0128588 |
| Example 179 | 0.00307539 | 0.0521315 |
| Example 180 | 0.00358344 | 0.0609116 |
| Example 181 | 0.00236211 | 0.0319742 |
| Example 182 | 0.00176401 | 0.0337831 |
| Example 183 | 0.00201326 | 0.034898 |
| Example 184 | 0.00174904 | 0.0140055 |
| Example 185 | 0.000366184 | <0.000838301 |
| Example 186 | | 0.0164135 |
| Example 187 | | 0.00407005 |
| Example 188 | | 0.0477529 |
| Example 189 | | 0.0367282 |
| Example 190 | | 0.0255094 |
| Example 191 | | 0.00251478 |
| Example 192 | | 0.0611927 |
| Example 193 | | 0.0234261 |
| Example 194 | | 0.0412477 |
| Example 195 | | 0.0115744 |
| Example 196 | | 0.00247002 |
| Example 197 | | 0.0194895 |
| Example 198 | | 0.00877203 |
| Example 199 | | 0.0502343 |
| Example 200 | | 0.0444529 |
| Example 201 | | 0.064121 |
| Example 202 | | 0.00758403 |
| Example 203 | | 0.0469461 |
| Example 204 | | 0.0869762 |
| Example 205 | | 0.0181218 |
| Example 206 | | 0.0175348 |
| Example 207 | | 0.0087781 |
| Example 208 | | 0.00855657 |
| Example 209 | | 0.0171356 |
| Example 210 | | 0.00306903 |
| Example 211 | | 0.0130137 |
| Example 212 | | 0.0472281 |
| Example 213 | | 0.0505359 |
| Example 214 | | 0.0324414 |
| Example 215 | | 0.00370169 |
| Example 216 | | 0.0310527 |
| Example 217 | | 0.027874 |
| Example 218 | | 0.00407099 |
| Example 219 | | 0.0496592 |
| Example 220 | | 0.014243 |
| Example 221 | | 0.00233507 |
| Example 222 | | 0.0118604 |
| Example 223 | | 0.0278356 |
| Example 224 | | 0.00127233 |
| Example 225 | | 0.00342768 |
| Example 226 | | 0.00126009 |
| Example 227 | | <0.000846838 |
| Example 228 | | 0.00166994 |
| Example 229 | | 0.00587489 |
| Example 230 | | 0.0377138 |
| Example 231 | | 0.00838881 |
| Example 232 | | 0.0245245 |
| Example 233 | | 0.00309315 |
| Example 234 | | 0.00836951 |
| Example 235 | | 0.0199388 |
| Example 236 | | 0.0172227 |
| Example 237 | | 0.0281774 |
| Example 238 | | 0.00225996 |
| Example 239 | | 0.00245076 |
| Example 240 | | 0.0160251 |
| Example 241 | | 0.0400959 |
| Example 242 | | 0.0447713 |
| Example 243 | | 0.035051 |
| Example 244 | | 0.0218022 |
| Example 245 | | 0.0239166 |
| Example 246 | | 0.0441367 |
| Example 247 | | 0.00711869 |
| Example 248 | | 0.00712361 |
| Example 249 | | 0.0237137 |
| Example 250 | | 0.021697 |
| Example 251 | | 0.00284315 |
| Example 252 | | 0.0284053 |
| Example 253 | | 0.00478741 |
| Example 254 | | 0.00636795 |
| Example 255 | | 0.00704207 |
| Example 256 | | 0.0198381 |
| Example 257 | | 0.00446067 |
| Example 258 | | 0.152897 |
| Example 259 | | 0.0152933 |
| Example 260 | | 0.0183316 |
| Example 261 | | 0.0238561 |
| Example 262 | | 0.00231207 |
| Example 263 | | 0.00843529 |
| Example 264 | | 0.0347616 |
| Example 265 | | 0.0036551 |
| Example 266 | | 0.00337521 |
| Example 267 | | 0.00331131 |
| Example 268 | | 0.00137563 |
| Example 269 | | 0.00153214 |
| Example 270 | | 0.0193152 |
| Example 271 | | 0.023383 |
| Example 272 | | 0.00875588 |
| Example 273 | | 0.0370084 |
| Example 274 | | 0.0290737 |
| Example 275 | | 0.0313328 |
| Example 276 | | 0.00921086 |
| Example 277 | | 0.0139187 |
| Example 278 | | 0.00250265 |
| Example 279 | | 0.00320332 |
| Example 280 | | 0.00320332 |
| Example 281 | | 0.00101812 |
| Example 282 | | 0.00607855 |
| Example 283 | | 0.00428647 |
| Example 284 | | 0.0187715 |
| Example 285 | | 0.0108718 |
| Example 286 | | 0.0165577 |
| Example 287 | | 0.00144477 |
| Example 288 | | 0.00695505 |
| Example 289 | | 0.00114025 |
| Example 290 | 0.00439036 | 0.0830616 |
| Example 291 | | 0.00808723 |
| Example 292 | | 0.0187111 |
| Example 293 | | <0.000846838 |
| Example 294 | | 0.0138452 |
| Example 295 | | 0.0176076 |
| Example 296 | | 0.00698232 |
| Example 297 | | 0.00781269 |
| Example 298 | | 0.0066696 |
| Example 299 | | 0.00674838 |
| Example 300 | | 0.00639146 |
| Example 301 | | 0.00773036 |
| Example 302 | | 0.000974541 |
| Example 303 | | 0.000971404 |
| Example 304 | | 0.00279898 |
| Example 305 | | 0.00116869 |
| Example 306 | | 0.0358096 |
| Example 307 | | 0.00131795 |
| Example 308 | | 0.00170962 |
| Example 309 | | <0.000846838 |
| Example 310 | | <0.000846838 |
| Example 311 | | 0.00570164 |
| Example 312 | | <0.000846838 |
| Example 313 | | 0.013807 |
| Example 314 | | 0.0128588 |
| Example 315 | | 0.00405789 |
| Example 316 | | <0.000846838 |
| Example 317 | | 0.00170334 |
| Example 318 | | <0.000846838 |
| Example 319 | | 0.00194267 |
| Example 320 | | 0.00101882 |
| Example 321 | | <0.000846838 |
| Example 322 | | 0.0015028 |
| Example 323 | | 0.0273149 |
| Example 324 | | 0.03287 |
| Example 325 | | 0.00264911 |
| Example 326 | | 0.0362076 |
| Example 327 | | 0.0366353 |
| Example 328 | | 0.0381241 |
| Example 329 | | 0.00358179 |
| Example 330 | | 0.028035 |
| Example 331 | | 0.0366016 |
| Example 332 | | 0.0149727 |
| Example 333 | | 0.0448023 |
| Example 334 | | 0.0030144 |
| Example 335 | | 0.0233453 |
| Example 336 | | <0.000846838 |
| Example 337 | | 0.00147503 |
| Example 338 | | 0.00324639 |
| Example 339 | | 0.00230888 |
| Example 340 | | <0.000846838 |
| Example 341 | | <0.000846838 |
| Example 342 | | 0.0320037 |
| Example 343 | | 0.0375751 |
| Example 344 | | 0.00361743 |
| Example 345 | | 0.0306408 |
| Example 346 | | 0.0166648 |
| Example 347 | | 0.0351722 |
| Example 348 | | <0.000846838 |
| Example 349 | | <0.000846838 |
| Example 350 | | 0.0403181 |
| Example 351 | | 0.00278099 |
| Example 352 | | 0.00309742 |
| Example 353 | | 0.0450816 |
| Example 354 | | 0.032181 |
| Example 355 | | 0.00680927 |
| Example 356 | | <0.000846838 |
| Example 357 | | 0.00101065 |
| Example 358 | | 0.00782888 |
| Example 359 | | 0.0271019 |
| Example 360 | | 0.0058023 |
| Example 361 | | 0.00703397 |
| Example 362 | | 0.0159625 |
| Example 363 | | 0.00349623 |
| Example 364 | | 0.0441266 |
| Example 365 | | <0.000846838 |
| Example 366 | | 0.00155883 |
| Example 367 | | 0.00681084 |
| Example 368 | | <0.000846838 |
| Example 369 | | 0.0016421 |
| Example 370 | | <0.000846838 |
| Example 371 | | <0.000846838 |
| Example 372 | | <0.000846838 |
| Example 373 | | <0.000846838 |
| Example 374 | | <0.000846838 |
| Example 375 | | 0.0011508 |
| Example 376 | | 0.00136301 |
| Example 377 | | <0.000846838 |
| Example 378 | | <0.000846838 |
| Example 379 | | 0.00124624 |
| Example 380 | | <0.000846838 |
| Example 381 | | <0.000846838 |
| Example 382 | | <0.000846838 |
| Example 383 | | 0.000947326 |
| Example 384 | | 0.000888792 |
| Example 385 | | 0.00119509 |
| Example 386 | | 0.00131159 |
| Example 387 | | 0.00100277 |
| Example 388 | | 0.00102589 |
| Example 389 | | 0.00102188 |
| Example 390 | | >50.0034 |
| Example 391 | | <0.000846838 |
| Example 392 | | 0.00106758 |
| Example 393 | | 0.00130527 |
| Example 394 | | <0.000846838 |
| Example 395 | | 0.000850746 |
| Example 396 | | 0.00109951 |
| Example 397 | | 0.00133352 |
| Example 398 | | <0.000846838 |
| Example 399 | | 0.0442385 |
| Example 400 | | 0.000912011 |
| Example 401 | | 0.0160842 |
| Example 402 | | 0.0983784 |
| Example 403 | | 0.0142626 |
| Example 404 | | 0.0579829 |
| Example 405 | | <0.000846838 |
| Example 406 | | 0.00183358 |
| Example 407 | | 0.00848789 |
| Example 408 | | 0.00100046 |
| Example 409 | | <0.000846838 |
| Example 410 | | <0.000846838 |
| Example 411 | | 0.00152265 |
| Example 412 | | 0.000883487 |
| Example 413 | | <0.000846838 |
| Example 414 | | 0.0152405 |
| Example 415 | | <0.000846838 |
| Example 416 | | <0.000846838 |
| Example 417 | | <0.000846838 |
| Example 418 | | <0.000846838 |
| Example 419 | | <0.000846838 |
| Example 420 | | 0.00236048 |
| Example 421 | | 0.0122208 |
| Example 422 | | <0.000846838 |
| Example 423 | | 0.0102849 |
| Example 424 | | 0.00766126 |
| Example 425 | | 0.00567675 |
| Example 426 | | 0.00403181 |
| Example 427 | 0.00120282 | 0.0114525 |
| Example 428 | 0.00201372 | 0.020797 |
| Example 429 | | 0.0121619 |
| Example 430 | | 0.00801124 |
| Example 431 | | 0.00193999 |
| Example 432 | 0.000344747 | 0.00171198 |
| Example 433 | | <0.000846838 |
| Example 434 | | <0.000846838 |
| Example 435 | | <0.000846838 |
| Example 436 | | <0.000846838 |
| Example 437 | | <0.000846838 |
| Example 438 | | 0.0101531 |
| Example 439 | | 0.00102873 |
| Example 440 | 0.000435011 | 0.00670502 |
| Example 441 | 0.0010639 | 0.012067 |
| Example 442 | 0.000323147 | 0.00374628 |
| Example 443 | | 0.00435512 |
| Example 444 | 0.000605202 | 0.00162817 |
| Example 445 | 0.00109295 | 0.0115638 |
| Example 446 | | 0.00318347 |
| Example 447 | | 0.024598 |
| Example 448 | | 0.0468705 |
| Example 449 | | 0.047152 |
| Example 450 | | 0.0399577 |
| Example 451 | | 0.0182222 |
| Example 452 | | 0.0149727 |
| Example 453 | | 0.0334041 |
| Example 454 | | 0.0134153 |
| Example 455 | | 0.0356205 |
| Example 456 | | 0.0203517 |
| Example 457 | | 0.0297715 |
| Example 458 | | 0.00555137 |
| Example 459 | | <0.000846838 |
| Example 460 | | 0.0412572 |
| Example 461 | | <0.000846838 |
| Example 462 | | 0.00740799 |
| Example 463 | | 0.0106881 |
| Example 464 | | 0.0116011 |
| Example 465 | | 0.0128647 |
| Example 466 | | 0.00115266 |
| Example 467 | | 0.0303949 |
| Example 468 | | 0.0081283 |
| Example 469 | | 0.00448023 |
| Example 470 | | 0.00511211 |
| Example 471 | | 0.027663 |
| Example 472 | | 0.026333 |
| Example 473 | | 0.018197 |
| Example 474 | | 0.0431221 |
| Example 475 | 0.00204692 | 0.0270832 |
| Example 476 | 0.00386278 | 0.0555777 |
| Example 477 | 0.00441571 | 0.0506757 |
| Example 478 | | <0.000846838 |
| Example 479 | | 0.037051 |
| Example 480 | | <0.000846838 |
| Example 481 | | <0.000846838 |
| Example 482 | | 0.00424131 |
| Example 483 | | 0.04432 |
| Example 484 | | 0.0571347 |
| Example 485 | | <0.000846838 |
| Example 486 | | 0.0284971 |
| Example 487 | | 0.0237794 |
| Example 488 | 0.002939 | 0.116118 |
| Example 489 | 0.000791408 | 0.0103443 |
| Example 490 | 0.00130017 | 0.0179184 |
| Example 491 | | 0.01516 |
| Example 492 | | 0.0516298 |
| Example 493 | | 0.0662217 |
| Example 494 | | 0.00253981 |
| Example 495 | | 0.0133999 |
| Example 496 | | 0.00143054 |
| Example 497 | | 0.0175833 |
| Example 498 | | 0.0414095 |
| Example 499 | | 0.00352452 |
| Example 500 | | 0.0453837 |
| Example 501 | | 0.00171791 |
| Example 502 | | 0.00682025 |
| Example 503 | 0.00380102 | 0.0682024 |
| Example 504 | | <0.000846838 |
| Example 505 | | 0.0120143 |
| Example 506 | | 0.0177705 |
| Example 507 | | 0.0297303 |
| Example 508 | 0.003661 | 0.0537032 |
| Example 509 | 0.00320848 | 0.0558599 |
| Example 510 | | 0.0614186 |
| Example 511 | | 0.0683282 |
| Example 512 | | 0.0346976 |
| Example 513 | | 0.0274663 |
| Example 514 | | 0.0303459 |
| Example 515 | | 0.00086437 |
| Example 516 | | 0.0528081 |
| Example 517 | | 0.0159772 |
| Example 518 | | 0.0558985 |
| Example 519 | | 0.0251478 |
| Example 520 | | 0.0444836 |
| Example 521 | | 0.0607155 |
| Example 522 | | 0.0130047 |
| Example 523 | | 0.00461849 |
| Example 524 | | 0.0203095 |
| Example 525 | | 0.0594429 |
| Example 526 | | 0.0198198 |
| Example 527 | | 0.00810401 |
| Example 528 | | 0.0641948 |
| Example 529 | | 0.000962719 |
| Example 530 | | 0.0023583 |
| Example 531 | | 0.00144212 |
| Example 532 | | 0.0134772 |
| Example 533 | | 0.005435 |
| Example 534 | | 0.0144577 |
| Example 535 | | 0.0107152 |
| Example 536 | | 0.0535427 |
| Example 537 | | 0.0240658 |
| Example 538 | | 0.0128558 |
| Example 539 | | 0.0605899 |
| Example 540 | | 0.0585194 |
| Example 541 | | 0.0470652 |
| Example 542 | | 0.0425011 |
| Example 543 | | 0.0159625 |
| Example 544 | | 0.014696 |
| Example 545 | | 0.0133721 |
| Example 546 | | 0.0238836 |
| Example 547 | | 0.0140184 |
| Example 548 | | 0.0462062 |
| Example 549 | | 0.0416485 |
| Example 550 | | 0.00550174 |
| Example 551 | | 0.00831572 |
| Example 552 | | 0.00378094 |
| Example 553 | | 0.0101042 |
| Example 554 | | 0.0275233 |
| Example 555 | | 0.00997471 |
| Example 556 | | 0.037051 |
| Example 557 | | 0.0494197 |
| Example 558 | | 0.0694864 |
| Example 559 | | 0.00685962 |
| Example 560 | | 0.0616169 |
| Example 561 | | 0.0618728 |
| Example 562 | 0.000438329 | 0.0119895 |
| Example 563 | | 0.0379927 |
| Example 564 | | 0.00338688 |
| Example 565 | 0.00383531 | 0.088247 |
| Example 566 | 0.00389045 | 0.101485 |
| Example 567 | 0.00756832 | 0.251247 |
| Example 568 | 0.00470761 | 0.093304 |
| Example 569 | | 0.0717464 |
| Example 570 | | 0.0943409 |
| Example 571 | | 0.164702 |
| Example 572 | | 0.0787409 |
| Example 573 | | 0.0740116 |
| Example 574 | | 0.0803526 |
| Example 575 | | 0.070291 |
| Example 576 | | 0.0727612 |
| Example 577 | | 0.0723936 |
| Example 578 | | 0.0998159 |
| Example 579 | | 0.0665886 |
| Example 580 | | 0.00198153 |
| Example 581 | | 0.0838494 |
| Example 582 | 0.00327793 | 0.0621871 |
| Example 583 | 0.00411813 | 0.061052 |
| Example 584 | 0.00253455 | 0.0353346 |
| Example 585 | 0.00148047 | 0.0169122 |
| Example 586 | 0.000950168 | 0.00862978 |
| Example 587 | 0.0038089 | 0.0870162 |
| Example 588 | 0.00342373 | 0.0595662 |
| Example 589 | 0.00625029 | 0.0759102 |
| Example 590 | 0.0012428 | 0.019476 |
| Example 591 | 0.000646547 | 0.00790133 |
| Example 592 | 0.00485959 | 0.120171 |
| Example 593 | 0.00242493 | 0.0307114 |
| Example 594 | 0.000626903 | 0.00908657 |
| Example 595 | 0.000791043 | 0.0179515 |
| Example 596 | 0.00124681 | 0.0146117 |
| Example 597 | 0.00192398 | 0.0299364 |
| Example 598 | 0.00409638 | 0.0534934 |
| Example 599 | | 0.141059 |
| Example 600 | 0.014171 | 0.207109 |
| Example 601 | | 0.115904 |
| Example 602 | | 0.113032 |
| Example 603 | | 0.202395 |
| Example 604 | | 0.202908 |
| Example 605 | | 0.315573 |
| Example 606 | | 0.100254 |
| Example 607 | | 0.100115 |
| Example 608 | | 0.0922146 |
| Example 609 | | 0.079726 |
| Example 610 | | 0.0829468 |
| Example 611 | | 0.116172 |
| Example 612 | | 0.114948 |
| Example 613 | | 0.100207 |
| Example 614 | | 0.087882 |
| Example 615 | 0.0117301 | |
| Example 616 | | 0.00102896 |
| Example 623 | | 0.00644021 |
| Example 624 | | 0.003287 |
| Example 625 | | 0.729794 |
| Example 626 | | 0.0862184 |
| Example 627 | | 0.0650878 |
| Example 628 | | 0.75544 |
| Example 629 | | 0.174502 |
| Example 630 | | 1.18304 |
| Example 631 | | 0.0771258 |
| Example 632 | | 0.00121143 |
| Example 633 | | 0.105293 |
| Example 634 | | 0.363413 |
| Example 635 | | 0.00196788 |
| Example 636 | | 0.0134555 |
| Example 637 | | 0.006418 |
| Example 638 | | 0.148525 |
| Example 639 | | 0.088736 |
| Example 640 | | 0.0294849 |
| Example 641 | | 0.110918 |
| Example 642 | | 0.00171238 |
| Example 643 | | 0.156747 |
| Example 644 | | 0.0734176 |
| Example 645 | | 0.325462 |
| Example 646 | | 0.0313328 |
| Example 647 | | 0.116869 |
| Example 648 | | 0.0886951 |
| Example 649 | | 0.166341 |
| Example 650 | | 0.0867561 |
| Example 651 | | 0.193598 |
| Example 652 | | 0.557442 |
| Example 653 | | 0.135863 |
| Example 654 | | 0.0742677 |
| Example 655 | | 0.0943409 |
| Example 656 | | 0.132251 |
| Example 657 | | 0.983559 |
| Example 659 | | 0.156567 |
| Example 660 | | 0.183654 |
| Example 661 | | 0.0748514 |
| Example 663 | 0.000150557 | <0.000838301 |
| Example 664 | | 0.0952577 |
| Example 665 | | 0.0244118 |
| Example 666 | | 0.0554754 |
| Example 667 | | 0.0127468 |
| Example 668 | | 0.31864 |
| Example 669 | | 0.0525654 |
| Example 670 | | 0.083138 |
| Example 671 | | 0.203657 |
| Example 672 | | 0.00400221 |
| Example 673 | | 0.00228613 |
| Example 674 | | 0.0629941 |
| Example 675 | | 0.0390571 |
| Example 676 | | 0.108094 |
| Example 677 | | 0.000935189 |
| Example 678 | | 0.0443404 |
| Example 679 | | 0.00319448 |
| Example 680 | | 0.0317468 |
| Example 681 | | 0.0347216 |
| Example 682 | | <0.000846838 |
| Example 683 | | 0.000973867 |
| Example 684 | | 0.0101695 |
| Example 685 | | 0.0311602 |
| Example 686 | | 0.0178649 |
| Example 687 | | 0.0758752 |
| Example 688 | | 0.00120781 |
| Example 689 | | 0.00135956 |
| Example 690 | | 0.00685173 |
| Example 691 | | 0.00100161 |
| Example 692 | | 0.00228034 |
| Example 693 | | 0.0765596 |
| Example 694 | | 0.0195389 |
| Example 695 | | 0.116547 |
| Example 696 | | 0.0821297 |
| Example 697 | | 0.0209652 |
| Example 698 | | 0.00180385 |
| Example 699 | | 0.011079 |
| Example 700 | | 0.00785597 |
| Example 701 | | 0.0279319 |
| Example 702 | | 0.00316519 |
| Example 703 | | 0.0698554 |
| Example 704 | | 0.0205636 |
| Example 705 | | 0.0306126 |
| Example 706 | | 0.0755092 |
| Example 707 | | 0.0056038 |
| Example 708 | | 0.0059126 |
| Example 709 | | 0.0030151 |
| Example 710 | | 0.0094005 |
| Example 711 | | 0.01004 |
| Example 712 | | 0.0041067 |
| Example 713 | | 0.0074555 |
| Example 714 | | 0.0061749 |
| Example 715 | | 0.011042 |
| Example 716 | | 0.011822 |
| Example 717 | | 0.0014193 |
| Example 718 | | 0.018161 |
| Example 719 | | 0.021598 |
| Example 720 | | 0.0095402 |
| Example 721 | | 0.026124 |
| Example 722 | | 0.010038 |
| Example 723 | | 0.0016279 |
| Example 724 | | 0.015533 |
| Example 725 | | 0.0040496 |
| Example 726 | | 0.0025062 |
| Example 727 | | 0.015189 |
| Example 728 | | 0.0049048 |
| Example 729 | | 0.019648 |
| Example 730 | | 0.015368 |
| Example 731 | | 0.0099712 |
| Example 732 | | 0.001638 |
| Example 733 | | 0.0019097 |
| Example 734 | | 0.0030657 |
| Example 735 | | 0.0091124 |
| Example 736 | | 0.0020551 |
| Example 737 | | 0.013977 |
| Example 738 | | 0.0030429 |
| Example 739 | | 0.0011895 |
| Example 740 | | 0.009437 |
| Example 741 | | 0.00266 |
| Example 742 | 0.4175 | |
| Example 743 | 0.02166 | |
| Example 744 | 0.2892 | |
| Example 745 | 3.52266 | |
| Example 746 | 0.2708 | |
| Example 747 | 0.03122 | |
| Example 748 | 0.09648 | |
| Example 749 | 0.01793 | 0.82096 |
| Example 750 | 0.4499 | |
| Example 751 | 0.1977 | |
| Example 752 | 0.06096 | 0.80594 |
| Example 753 | 0.002976 | 0.48311 |
| Example 754 | | 0.071048 |
| Example 755 | | 0.002149 |
| Example 756 | | 0.27516 |

1. A compound of formula (I): 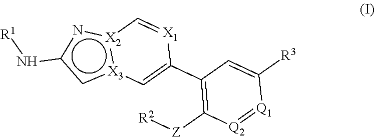
or a stereoisomer, tautomer, pharmaceutically acceptable salt or solvate thereof,
wherein:
X₁, X₂, and X₃ are independently N or CH;
Q₁ and Q₂ are independently N or CH;
Z is —O—Cₙ₁H₂ₙ₁—, or —NR⁶—(CH₂)ₙ₂—, provided that O or N is directly bonded to the pyridinyl ring or phenyl ring or pyridazinyl ring;
n1 is 0, 1 or 2, and 2n1 is twice of n1;
n2 is 0 or 1;
R¹ is —C(O)R⁵, phenyl, a 5- to 6-membered heteroaryl having 1-3 nitrogen heteroatoms, or 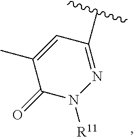
wherein each of the phenyl and 5- to 6-membered heteroaryl is optionally substituted with one or more substituents independently selected from the group consisting of C₁-C₄ alkyl, —F, C₁ fluoroalkyl, C₁₋₄ hydroxyalkyl, —CON(R⁸)₂, —COOR¹³, —C(O)R⁹, cyclopropyl, —CH₂OCH₃, —N(R¹⁰)₂, —OCH₃, —OCHF₂, —SO₂CH₃, —SO₂N(R¹⁴)₂, and a 4- to 9-membered monocyclic having 1 to 2 heteroatoms independently selected from oxygen and nitrogen, wherein the C₁-C₄ alkyl is optionally substituted with one or more substituents independently selected from the group consisting of —F, —OH and morpholino, wherein the 5- to 6-membered heteroaryl is optionally fused to a ring selected from the group consisting of benzene, piperidine, piperazine, and a 5- to 6-membered heteroaryl having 1-2 nitrogen heteroatoms, wherein the 5- to 6-membered heteroaryl is optionally substituted with one or more —CH₃;
R⁵ is cyclopropyl optionally substituted with one or more substituents independently selected from the group consisting of —F, —CH₃, C₁ fluoroalkyl, and phenyl;
R³ is hydrogen, —CH₃, —CH₂CH₃, propynyl, C₁ fluoroalkyl, —CH₂OCH₃, —COOCH₂CH₃, —CN, —Cl, —N(R¹⁰)₂, or —OR¹⁵;
R² is:
a 4- to 7-membered monocyclic heterocyclyl selected from the group consisting of pyrrolidinyl, piperidinyl, oxazepanyl, morpholinyl, azetidinyl, tetrahydrofuryl, tetrahydropyranyl, oxetanyl, and 1,1-dioxo-1,2,5-thiadiazolidinyl, wherein the 4- to 7-membered monocyclic heterocyclyl is optionally substituted with one or more substituents independently selected from the group consisting of —CH₃, —CD₃, —OCH₃, —CH₂CH₃, —CH(CH₃)₂, —CH₂CF₃, —CF₃, —CH₂OH, —CH₂C(OH)(CH₃)₂, —F, —OH, —NH₂, —CN, oxo, cyclopropyl, —COOC(CH₃)₃, —CONH₂, —C(O)CH₃, —CH₂CN, —CH₂CH₂OCH₃, —CH₂(C₅H₄N), —SO₂CH₃, and phenyl,
a 6- to 9-membered bicyclic heterocyclyl selected from the group consisting of 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonanyl, 6-oxaspiro[3.4]octanyl, and 1-oxaspiro[4.4]nonanyl, wherein the 6- to 9-membered bicyclic heterocyclyl is optionally substituted with one or more substituents independently selected from the group consisting of —CH₃, —CD₃, —CH₂CH₃, —CH(CH₃)₂, —CH₂C(OH)(CH₃)₂, —COOC(CH₃)₃, —CN, —SO₂CH₃, oxo, 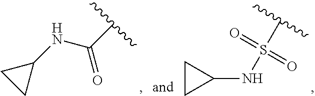 [SMILES: *C(=O)NC1CC1 ; *S(=O)(=O)NC1CC1]
a C₃-C₆ cycloalkyl, wherein the C₃-C₆ cycloalkyl is optionally substituted with one or more substituents independently selected from the group consisting of —NH₂, —OH, —OCH₃, —CH₂OH, ═O, —CONH₂, phenyl, —CH₃, —CF₃, —F, —NHCH₃, —NH(C₃H₅), —NHCH₂CN, —NH—COOCH₂CH₃, —NH(C₅H₄N), —NH(C₆H₅), 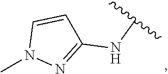 [SMILES: *Nc1ccn(C)n1]
═NH(OH), —SO₂CH₃, and —CH(CH₃)₂,
a C₁₋₅ alkyl optionally substituted with one or more substituents independently selected from the group consisting of —N(CH₃)₂, —OH, —CF₃, —NH₂, —COOC(CH₃)₃, —NH—COCH₃, —NH—SO₂CH₃, —COONH₂, —COON(CH₃)₂, cyclopropyl, methylcyclopropyl, —CH₃, C₃-C₆ cycloalkyl, —SO₂CH₃, phenyl, pyridyl, and —CN,
pyridinyl or pyrazolyl, wherein each of the pyridinyl and pyrazolyl is optionally substituted with one or more substituents independently selected from the group consisting of —CF₃ and —CH₃,
or phenyl, or phenyl substituted with —OCHF₂,
R⁶ is hydrogen,
or alternatively, when Z is —NR⁶—(CH₂)ₙ₂— and n2 is 0, R² and R⁶ are taken together with the nitrogen atom to which they are attached to form a 4- to 8-membered monocyclic or bicyclic heterocyclyl having 1-2 nitrogen heteroatoms, wherein the 4- to 8-membered monocyclic or bicyclic heterocyclyl is optionally substituted with one or more substituents independently selected from the group consisting of —OH, C¹ fluoroalkyl, C₁₋₃ hydroxyalkyl, —C(O)N(R¹⁰)₂ and —CH₂N(R¹⁰)₂;
each R⁸ is independently hydrogen, cyclopropyl, tetrahydropyran, or C₁₋₄ alkyl, wherein the C₁₋₄ alkyl is optionally substituted with one or more substituents independently selected from the group consisting of —OH, —OCH₃, —F, tetrahydropyran, and —CN;
each R⁹ is independently C₁ fluoroalkyl or —CH₃;
each R¹⁰ is independently hydrogen, —COCH₃, —SO₂CH₃, C₁₋₂ alkyl, or C₁₋₂ fluoroalkyl;
each R¹¹ is independently hydrogen, C₁₋₅ alkyl, —CH₂CH₂—OCHF₂, cyclopropyl, or —CH₂-cyclopropyl;
each R¹³ is independently hydrogen or C₁₋₄ alkyl;
each R¹⁴ is independently cyclopropyl or C₁₋₄ alkyl; and
each R¹⁵ is independently hydrogen or C₁₋₄ alkyl, wherein the C₁₋₄ alkyl is optionally substituted with one or more substituents independently selected from the group consisting of —OH, —F, and —CN.

2. The compound of claim 1, wherein R¹ is —C(O)R⁵ and R⁵ is cyclopropyl.

3. The compound of claim 1, wherein R¹ is:
triazolyl, pyrazolyl, pyrazinyl, pyridazinyl, pyrimidinyl, pyridinyl, pyrrolo[2,3-b]pyridinyl, imidazo[1,2-b]pyridazinyl, indazolyl, 4,5,6,7-tetrahydropyrazolo[1,5-a]pyrazinyl, 5,6,7,8-tetrahydro-2,7-naphthyridinyl, or 5,6,7,8-tetrahydro-2,6-naphthyridinyl, wherein each of the triazolyl, pyrazolyl, pyrazinyl, pyridazinyl, pyrimidinyl, pyrrolo[2,3-b]pyridinyl, imidazo[1,2-b]pyridazinyl, indazolyl, 4,5,6,7-tetrahydropyrazolo[1,5-a]pyrazinyl, 5,6,7,8-tetrahydro-2,7-naphthyridinyl, 5,6,7,8-tetrahydro-2,6-naphthyridinyl, and pyridinyl is optionally substituted with one or more substituents independently selected from the group consisting of —C(O)NHCH₃, —F, —CH₃, —CH₂CH₃, —NHCH₃, —CHF₂, —C(O)CF₃, —CH₂C(OH)(CH₃)₂, —CH(CH₃)₂, C(CH₃)₃, —C₃H₅, —C(O)N(CH₃)₂, —CH₂OCH₃, —CH(OH)CHCF₃, —CO₂H, —CO₂CH₃, —SO₂CH₃, —SO₂N(CH₃)₂, 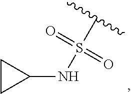 [SMILES: *S(=O)(=O)NC1CC1]
cyclopropyl, pyrrolidinyl, morpholino, and morpholinomethyl; 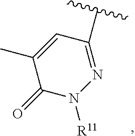
wherein R¹¹ is hydrogen, —CH₃, —CH₂CH₃, —CH₂CH₂—OCHF₂, cyclopropyl, or —CH₂-cyclopropyl; or
phenyl, wherein the phenyl is optionally substituted with one or more substituents independently selected from the group consisting of —COOCH₃, —C(O)N(H)CH₂CH₃, —C(O)N(H)CH₂CF₃, —OCH₃, —OCHF₂, 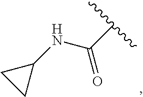 [SMILES: *C(=O)NC1CC1]
—SO₂N(CH₃)₂, and

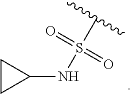 [SMILES: *S(=O)(=O)NC1CC1]

4. The compound of claim 1, wherein R¹ is:

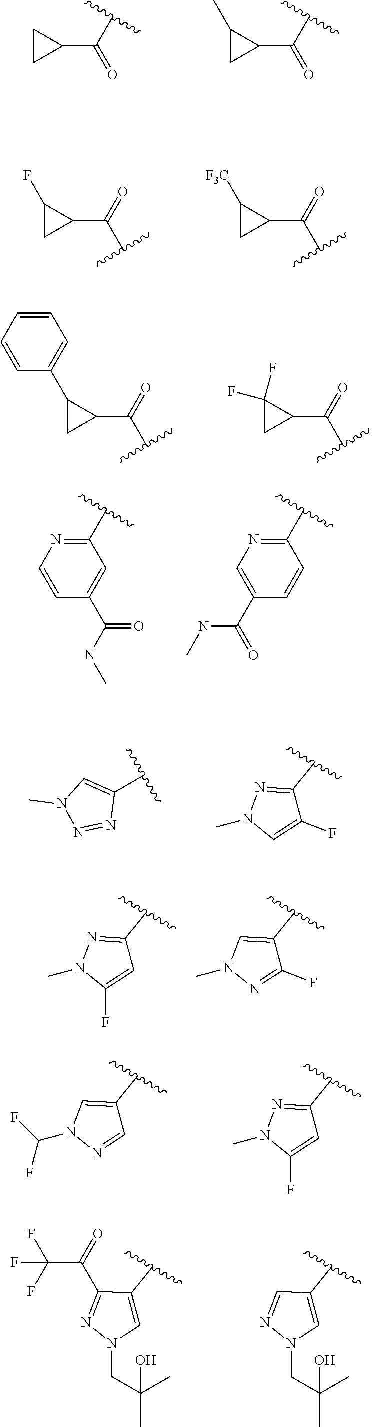

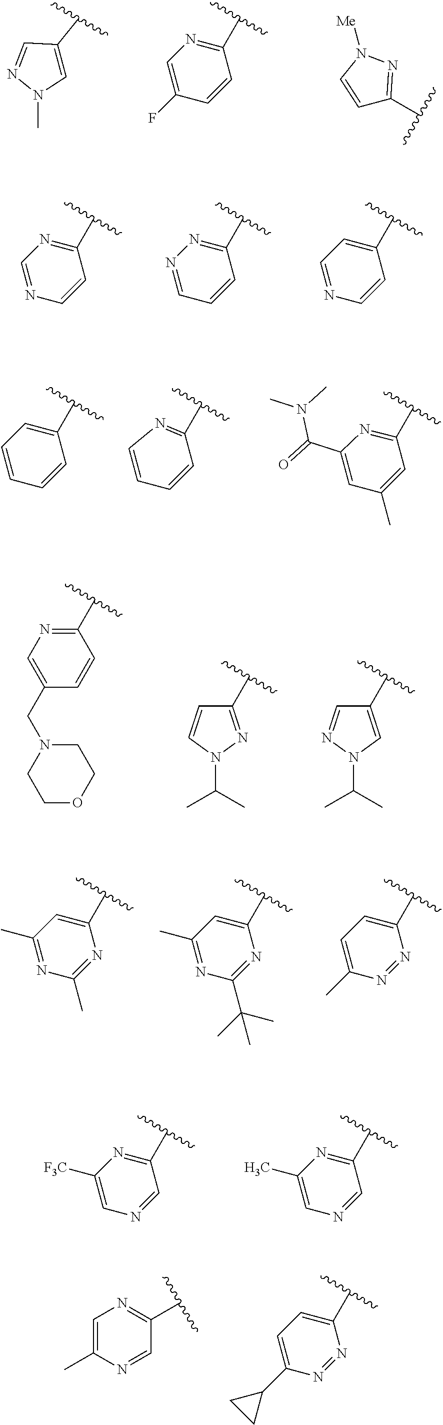

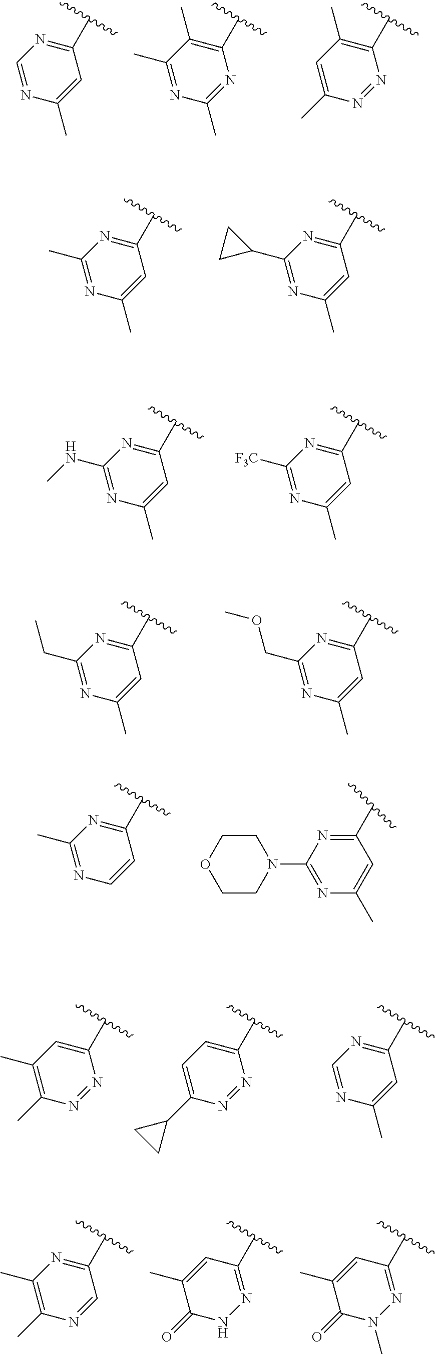

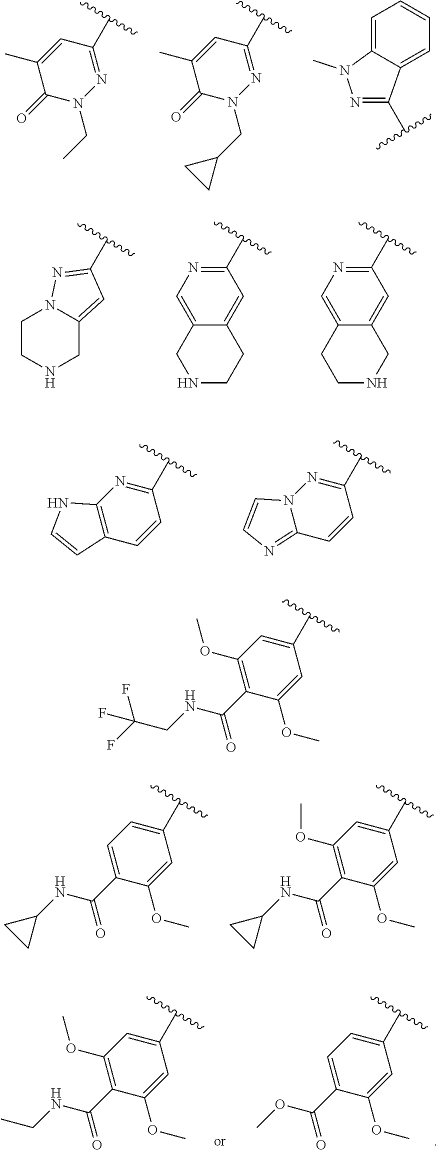

5. The compound of claim 1, wherein Z is —O—, —O—CH₂—, —O—CH(CH₃)—, or —O—CH₂—CH₂—.

6. The compound of claim 1, wherein R² is a 4- to 7-membered monocyclic heterocyclyl selected from the group consisting of pyrrolidinyl, piperidinyl, oxazepanyl, morpholinyl, azetidinyl, tetrahydrofuryl, tetrahydropyranyl, oxetanyl, and 1,1-dioxo-1,2,5-thiadiazolidinyl, wherein the 4- to 7-membered monocyclic heterocyclyl is optionally substituted with one or more substituents independently selected from the group consisting of —CH₃, —CD₃, —OCH₃, —CH₂CH₃, —CH(CH₃)₂, —CH₂CF₃, —CF₃, —CH₂OH, —CH₂C(OH)(CH₃)₂, —F, —OH, —NH₂, —CN, oxo, cyclopropyl, —COOC(CH₃)₃, —CONH₂, —C(O)CH₃, —CH₂CN, —CH₂CH₂OCH₃, —CH₂(C₅H₄N), —SO₂CH₃, and phenyl.

7. The compound of claim 1, wherein R² is a 6- to 9-membered bicyclic heterocyclyl selected from the group consisting of 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonanyl, 6-oxaspiro[3.4]octanyl, and 1-oxaspiro[4.4]nonanyl, wherein the 6- to 9-membered bicyclic heterocyclyl is optionally substituted with one or more substituents independently selected from the group consisting of —CH₃, —CD₃, —CH₂CH₃, —CH(CH₃)₂, —CH₂C(OH)(CH₃)₂, —COOC(CH₃)₃, —CN, —SO₂CH₃, oxo,

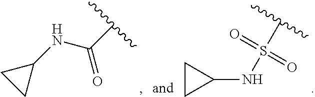 [SMILES: *C(=O)NC1CC1 ; *S(=O)(=O)NC1CC1]

8. The compound of claim 1, wherein R² is a C₃-C₆ cycloalkyl, wherein the C₃-C₆ cycloalkyl is optionally substituted with one or more substituents independently selected from the group consisting of —NH₂, —OH, —OCH₃, —CH₂OH, ═O, —CONH₂, phenyl, —CH₃, —CF₃, —F, —NHCH₃, —NH(C₃H₅), —NHCH₂CN, —NH—COOCH₂CH₃, —NH(C₅H₄N), —NH(C₆H₅), 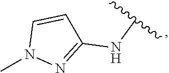 [SMILES: *Nc1ccn(C)n1]
═NH(OH), —SO₂CH₃, and —CH(CH₃)₂.

9. The compound of claim 1, wherein R² is a C₁₋₅ alkyl optionally substituted with one or more substituents independently selected from the group consisting of —N(CH₃)₂, —OH, —CF₃, —NH₂, —COOC(CH₃)₃, —NH—COCH₃, —NH—SO₂CH₃, —COONH₂, —COON(CH₃)₂, cyclopropyl, methylcyclopropyl, —CH₃, C₃-C₆ cycloalkyl, —SO₂CH₃, phenyl, pyridyl, and —CN.

10. The compound of claim 1, wherein R² is pyridinyl or pyrazolyl, wherein each of the pyridinyl and pyrazolyl is optionally substituted with one or more substituents independently selected from the group consisting of —CF₃ and —CH₃.

11. The compound of claim 1, wherein:
Z is —NR⁶—(CH₂)ₙ₂;
n2 is 0; and
R² and R⁶ are taken together to form a 4- to 8-membered monocyclic or bicyclic heterocyclyl selected from the group consisting of: 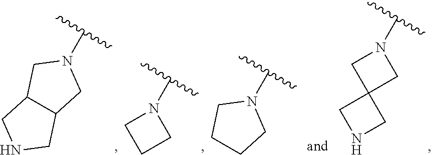
wherein the 4- to 8-membered monocyclic or bicyclic heterocyclyl is optionally substituted with one or more substituents independently selected from the group consisting —OH, —CH₂OH, —CH₂N(CH₃)₂, —C(CH₃)₂OH, —CF₃, and —C(O)NHCH₃.

12. The compound of claim 1, being a compound of formula (I-A): 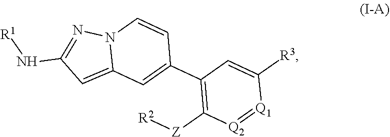
or a stereoisomer, tautomer, pharmaceutically acceptable salt or solvate thereof,
wherein Z, Q₁, Q₂, R¹, R², and R³ are defined as in claim 1.

13. The compound of formula 12, wherein Q₁ is N, and Q₂ is CH.

14. The compound of formula 12, wherein Q₁ is CH, and Q₂ is CH.

15. The compound of formula 12, wherein Q₁ is N, and Q₂ is N.

16. The compound of claim 1, being a compound of formula (I-B): 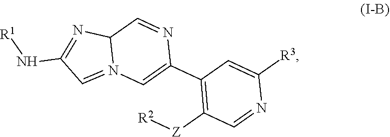
or a stereoisomer, tautomer, pharmaceutically acceptable salt or solvate thereof,
wherein Z, R¹, R², and R³ are defined as in claim 1.

17. A compound selected from the group consisting of:
N-[5-[2-Cyano-5-[[(3R)-1-methylpyrrolidin-3-yl]methoxy]-4-pyridyl]pyrazolo[1,5-a]pyridin-2-yl]cyclopropanecarboxamide;
N-[5-[2-cyano-5-(2-dimethylaminoethylamino)-4-pyridyl]pyrazolo[1,5-a]pyridin-2-yl]cyclopropanecarboxamide;
N-[5-[2-cyano-5-[3-(hydroxymethyl)azetidin-1-yl]-4-pyridyl]pyrazolo[1,5-a]pyridin-2-yl]cyclopropanecarboxamide;
(R)-5-methylpyrrolidin-3-yl)methoxy)-4-(2-(pyridin-3-ylamino)pyrazolo[1,5-a]pyridin-5-yl) picolinonitrile;
N-[5-[2-cyano-5-[(3S)-1-(2,2,2-trifluoroethyl)pyrrolidin-3-yl]oxy-4-pyridyl]pyrazolo[1,5-a]pyridin-2-yl]cyclopropanecarboxamide;
(S)—N-(5-(2-cyano-5-(pyrrolidin-3-yloxy)pyridin-4-yl)pyrazolo[1,5-a]pyridin-2-yl)cyclopropanecarboxamide
N-[5-[2-cyano-5-[(3S)-1-cyclopropylpyrrolidin-3-yl]oxy-4-pyridyl]pyrazolo[1,5-a]pyridin-2-yl]cyclopropanecarboxamide
N-(5-(2-cyano-5-(((2*S,4*R)-2-(trifluoromethyl)piperidin-4-yl)oxy)pyridin-4-yl)pyrazolo[1,5-a]pyridin-2-yl)cyclopropanecarboxamide
N-(5-(2-cyano-5-((4-(trifluoromethyl)piperidin-4-yl)oxy)pyridin-4-yl)pyrazolo[1,5-a]pyridin-2-y 1)cyclopropanecarboxamide;
N-[5-[2-cyano-5-[[1-methyl-4-(trifluoromethyl)-4-piperidyl]oxy]-4-pyridyl]pyrazolo[1,5-a]pyridin-2-yl]cyclopropanecarboxamide;
N-[5-[2-cyano-5-[[(4R)-3,3-difluoro-1-methyl-4-piperidyl]oxy]-4-pyridyl]pyrazolo[1,5-a]pyridin-2-yl]cyclopropanecarboxamide;
N-[5-[2-cyano-5-[[(6R)-4-methyl-1,4-oxazepan-6-yl]oxy]-4-pyridyl]pyrazolo[1,5-a]pyridin-2-yl]cyclopropanecarboxamide;
N-[5-[2-cyano-5-[[(1R,4R,5R)-2-methyl-2-azabicyclo[2.2.1]heptan-5-yl]oxy]-4-pyridyl]pyrazolo[1,5-a]pyridin-2-yl]cyclopropanecarboxamide;
N-(5-(2-cyano-5-(((2*R,4*S)-2-(trifluoromethyl)piperidin-4-yl)oxy)pyridin-4-yl)pyrazolo[1,5-a]pyridin-2-yl)cyclopropanecarboxamide;
N-[5-[2-cyano-5-[[(4R)-3,3-difluoro-4-piperidyl]oxy]-4-pyridyl]pyrazolo[1,5-a]pyridin-2-yl]cyclopropanecarboxamide;
(S)—N-(5-(2-cyano-5-methylpyrrolidin-3-yl)methoxy)pyridin-4-yl)pyrazolo[1,5-a]pyridin-2-y 1)cyclopropanecarboxamide;
N-[5-[5-[[(1*S,4*S,5*R)-2-azabicyclo[2.2.1]heptan-5-yl]oxy]-2-cyano-4-pyridyl]pyrazolo[1,5-a]pyridin-2-yl]cyclopropanecarboxamide;
N-[5-[5-[[(1*R,4*R,5*S)-2-azabicyclo[2.2.1]heptan-5-yl]oxy]-2-cyano-4-pyridyl]pyrazolo[1,5-a]pyridin-2-yl]cyclopropanecarboxamide;
N-[5-[2-cyano-5-[[(2S)-morpholin-2-yl]methoxy]-4-pyridyl]pyrazolo[1,5-a]pyridin-2-yl]cyclopropanecarboxamide;
N-[5-[5-[[(1R,3S)-3-aminocyclopentyl]methoxy]-2-cyano-4-pyridyl]pyrazolo[1,5-a]pyridin-2-yl]cyclopropanecarboxamide;
N-[5-[2-cyano-5-[[(6R)-1,4-oxazepan-6-yl]oxy]-4-pyridyl]pyrazolo[1,5-a]pyridin-2-yl]cyclopropanecarboxamide;
N-[5-[5-[[(1R,3R)-3-aminocyclopentyl]methoxy]-2-cyano-4-pyridyl]pyrazolo[1,5-a]pyridin-2-yl]cyclopropanecarboxamide;
N-[5-[5-(azetidin-3-ylmethoxy)-2-cyano-4-pyridyl]pyrazolo[1,5-a]pyridin-2-yl]cyclopropanecarboxamide;
N-[5-[5-[[(1R,4R,5R)-2-azabicyclo[2.2.1]heptan-5-yl]oxy]-2-cyano-4-pyridyl]pyrazolo[1,5-a]pyridin-2-yl]cyclopropanecarboxamide;
N-[5-[2-cyano-5-[(3R)-1-cyclopropylpyrrolidin-3-yl]oxy-4-pyridyl]pyrazolo[1,5-a]pyridin-2-yl]cyclopropanecarboxamide;
N-[5-[2-cyano-5-[[(6S)-4-methyl-1,4-oxazepan-6-yl]oxy]-4-pyridyl]pyrazolo[1,5-a]pyridin-2-yl]cyclopropanecarboxamide;
N-[5-[2-cyano-5-[[(1S,5R)-8-methyl-8-azabicyclo[3.2.1]octan-3-yl]methoxy]-4-pyridyl]pyrazolo[1,5-a]pyridin-2-yl]cyclopropanecarboxamide;
N-[5-[2-cyano-5-[[(3S,5R)-1-methyl-5-(trifluoromethyl)-3-piperidyl]oxy]-4-pyridyl]pyrazolo[1,5-a]pyridin-2-yl]cyclopropanecarboxamide;
N-(5-(2-cyano-5-(((2*S,4*R)-1-methyl-2-(trifluoromethyl)piperidin-4-yl)oxy)pyridin-4-yl)pyrazolo[1,5-a]pyridin-2-yl)cyclopropanecarboxamide;
N-(5-(2-cyano-5-(((2*R,4*S)-1-methyl-2-(trifluoromethyl)piperidin-4-yl)oxy)pyridin-4-yl)pyrazolo[1,5-a]pyridin-2-yl)cyclopropanecarboxamide;
N-[5-[5-(azetidin-3-yloxy)-2-cyano-4-pyridyl]pyrazolo[1,5-a]pyridin-2-yl]cyclopropanecarboxamide;
N-[5-[2-cyano-5-[(1-methylazetidin-3-yl)methoxy]-4-pyridyl]pyrazolo[1,5-a]pyridin-2-yl]cyclopropanecarboxamide;
rac-N-(5-(2-cyano-5-((5-oxopyrrolidin-3-yl)methoxy)pyridin-4-yl)pyrazolo[1,5-a]pyridin-2-yl)cyclopropanecarboxamide;
tert-butyl 2-[[6-cyano-4-[2-(cyclopropanecarbonylamino)pyrazolo[1,5-a]pyridin-5-yl]-3-pyridyl]oxy]-6-azaspiro[3.3]heptane-6-carboxylate;
tert-butyl (3R)-3-[[6-cyano-4-[2-(cyclopropanecarbonylamino)pyrazolo[1,5-a]pyridin-5-yl]-3-pyridyl]oxy methyl]pyrrolidine-1-carboxylate;
(R)—N-(5-(2-cyano-5-((4-methylmorpholin-2-yl)methoxy)pyridin-4-yl)pyrazolo[1,5-a]pyridin-2-yl)cyclopropanecarboxamide;
(R)—N-(5-(2-cyano-5-((1-methylpiperidin-3-yl)methoxy)pyridin-4-yl)pyrazolo[1,5-a]pyridin-2-yl) cyclopropanecarboxamide;
(S)—N-(5-(2-cyano-5-((1-methylpiperidin-3-yl)methoxy)pyridin-4-yl)pyrazolo[1,5-a]pyridin-2-yl) cyclopropanecarboxamide;
N-[5-[2-cyano-5-[[(2S)-4-methylmorpholin-2-yl]methoxy]-4-pyridyl]pyrazolo[1,5-a]pyridin-2-yl]cyclopropanecarboxamide;
N-[5-[2-cyano-5-[(2-oxo-3-azabicyclo[2.2.2]octan-4-yl)methoxy]-4-pyridyl]pyrazolo[1,5-a]pyridin-2-yl]cyclopropanecarboxamide;
N-[5-[2-cyano-5-[(1-hydroxycyclobutyl)methoxy]-4-pyridyl]pyrazolo[1,5-a]pyridin-2-yl]cyclopropanecarboxamide;
N-[5-[2-cyano-5-[(1-hydroxycyclopentyl)methoxy]-4-pyridyl]pyrazolo[1,5-a]pyridin-2-yl]cyclopropanecarboxamide;
N-[5-[2-cyano-5-(4-piperidylmethoxy)-4-pyridyl]pyrazolo[1,5-a]pyridin-2-yl]cyclopropanecarboxamide;
N-[5-[5-[(1-aminocyclobutyl)methoxy]-2-cyano-4-pyridyl]pyrazolo[1,5-a]pyridin-2-yl]cyclopropanecarboxamide;
N-[5-[2-Cyano-5-[[(3R)-pyrrolidin-3-yl]methoxy]-4-pyridyl]pyrazolo[1,5-a]pyridin-2-yl]cyclopropanecarboxamide;
N-[5-[2-Cyano-5-(cyclopropoxy)-4-pyridyl]pyrazolo[1,5-a]pyridin-2-yl]cyclopropanecarboxamide;
N-[5-[2-Cyano-5-(3-pyridylmethoxy)-4-pyridyl]pyrazolo[1,5-a]pyridin-2-yl]cyclopropanecarboxamide;
N-(5-(2-Cyano-5-(pyridin-2-ylmethoxy)pyridin-4-yl)pyrazolo[1,5-a]pyridin-2-yl)cyclopropanecarboxamide;
(*R)—N-(5-(2-Cyano-5-(3,3,3-trifluoro-2-hydroxy-2-methylpropoxy)pyridin-4-yl)pyrazolo[1,5-a]pyridin-2-yl)cyclopropanecarboxamide;
(*S)—N-(5-(2-Cyano-5-(3,3,3-trifluoro-2-hydroxy-2-methylpropoxy)pyridin-4-yl)pyrazolo[1,5-a]pyridin-2-yl)cyclopropanecarboxamide;
N-[5-[2-cyano-5-[(1-methyl-4-piperidyl)methoxy]-4-pyridyl]pyrazolo[1,5-a]pyridin-2-yl]cyclopropanecarboxamide;
N-[5-[2-Cyano-5-(2-hydroxy-2-methyl-propoxy)-4-pyridyl]pyrazolo[1,5-a]pyridin-2-yl]cyclopropanecarboxamide;
trans-N-(5-(2-Cyano-5-(((1r,4r)-4-hydroxycyclohexyl)methoxy)pyridin-4-yl)pyrazolo[1,5-a]pyridin-2-yl)cyclopropanecarboxamide;
N-[5-[2-Cyano-5-(1-methylazetidin-3-yl)oxy-4-pyridyl]pyrazolo[1,5-a]pyridin-2-yl]cyclopropanecarboxamide;
tert-butyl 2-[[6-Cyano-4-[2-(cyclopropanecarbonylamino)pyrazolo[1,5-a]pyridin-5-yl]-3-pyridyl]oxy]acetate;
(*R)—N-(5-(5-((1-Acetylpyrrolidin-3-yl)oxy)-2-cyanopyridin-4-yl)pyrazolo[1,5-a]pyridin-2-yl)cyclopropanecarboxamide;
(*S)—N-(5-(5-((1-Acetylpyrrolidin-3-yl)oxy)-2-cyanopyridin-4-yl)pyrazolo[1,5-a]pyridin-2-yl)cyclopropanecarboxamide;
(*R)—N-(5-(2-cyano-5-((1-methylpyrrolidin-3-yl)oxy)pyridin-4-yl)pyrazolo[1,5-a]pyridin-2-yl)cyclopropanecarboxamide;
(*S)—N-(5-(2-cyano-5-((1-methylpyrrolidin-3-yl)oxy)pyridin-4-yl)pyrazolo[1,5-a]pyridin-2-yl)cyclopropanecarboxamide;
N-[5-[5-[(1-acetyl-4-piperidyl)methoxy]-2-cyano-4-pyridyl]pyrazolo[1,5-a]pyridin-2-yl]cyclopropanecarboxamide;
N-[5-[2-cyano-5-[[6-(trifluoromethyl)-3-pyridyl]methoxy]-4-pyridyl]pyrazolo[1,5-a]pyridin-2-yl]cyclopropanecarboxamide;
N-[5-[2-cyano-5-[[2-(trifluoromethyl)-4-pyridyl]methoxy]-4-pyridyl]pyrazolo[1,5-a]pyridin-2-yl]cyclopropanecarboxamide;
N-[5-[2-cyano-5-[(2S)-2-hydroxypropoxy]-4-pyridyl]pyrazolo[1,5-a]pyridin-2-yl]cyclopropanecarboxamide;
N-[5-[2-cyano-5-(4-hydroxycyclohexoxy)-4-pyridyl]pyrazolo[1,5-a]pyridin-2-yl]cyclopropanecarboxamide;
N-[5-[2-cyano-5-[[(3S)-1-methyl-3-piperidyl]oxy]-4-pyridyl]pyrazolo[1,5-a]pyridin-2-yl]cyclopropanecarboxamide;
N-[5-[2-cyano-5-[[(1S,5R)-8-methyl-8-azabicyclo[3.2.1]octan-3-yl]oxy]-4-pyridyl]pyrazolo[1,5-a]pyridin-2-yl]cyclopropanecarboxamide;
N-[5-[5-[[(1S,5R)-8-azabicyclo[3.2.1]octan-3-yl]methoxy]-2-cyano-4-pyridyl]pyrazolo[1,5-a]pyridin-2-yl]cyclopropanecarboxamide;
N-[5-[2-cyano-5-[(3-hydroxycyclobutyl)methoxy]-4-pyridyl]pyrazolo[1,5-a]pyridin-2-yl]cyclopropanecarboxamide;
N-[5-[2-cyano-5-(hydroxymethyl)cyclobutoxy]-4-pyridyl]pyrazolo[1,5-a]pyridin-2-yl]cyclopropanecarboxamide;
N-[5-[2-cyano-5-(3-hydroxyazetidin-1-yl)-4-pyridyl]pyrazolo[1,5-a]pyridin-2-yl]cyclopropanecarboxamide;
N-[5-[2-cyano-5-[[(3R)-1-methylpyrrolidin-3-yl]methylamino]-4-pyridyl]pyrazolo[1,5-a]pyridin-2-yl]cyclopropanecarboxamide;
N-[5-[2-cyano-5-[3-(dimethylaminomethyl)azetidin-1-yl]-4-pyridyl]pyrazolo[1,5-a]pyridin-2-yl]cyclopropanecarboxamide;
1-[6-cyano-4-[2-(cyclopropanecarbonylamino)pyrazolo[1,5-a]pyridin-5-yl]-3-pyridyl]-N-methyl-azetidine-3-carboxamide;
N-[5-[2-cyano-5-[3-(1-hydroxy-1-methyl-ethyl)azetidin-1-yl]-4-pyridyl]pyrazolo[1,5-a]pyridin-2-yl]cyclopropanecarboxamide;
N-[5-[2-cyano-5-[3-hydroxy-3-(trifluoromethyl)azetidin-1-yl]-4-pyridyl]pyrazolo[1,5-a]pyridin-2-yl]cyclopropanecarboxamide;
N-[5-[2-cyano-5-(cyclopropylmethylamino)-4-pyridyl]pyrazolo[1,5-a]pyridin-2-yl]cyclopropane carboxamide;
N-[5-[2-cyano-5-[(1-methylpyrazol-3-yl)amino]-4-pyridyl]pyrazolo[1,5-a]pyridin-2-yl]cyclopropanecarboxamide;
N-[5-[2-cyano-5-(cyclopentylamino)-4-pyridyl]pyrazolo[1,5-a]pyridin-2-yl]cyclopropanecarboxamide;
N-[5-[2-cyano-5-[(1-methylpyrazol-4-yl)amino]-4-pyridyl]pyrazolo[1,5-a]pyridin-2-yl]cyclopropanecarboxamide;
N-[5-[2-cyano-5-(cyclopropylamino)-4-pyridyl]pyrazolo[1,5-a]pyridin-2-yl]cyclopropanecarboxamide;
N-[5-[2-cyano-5-[(2-hydroxy-2-methyl-propyl)amino]-4-pyridyl]pyrazolo[1,5-a]pyridin-2-yl]cyclopropanecarboxamide;
(3-endo)-N-(5-(5-(((1R,3r,5S)-8-azabicyclo[3.2.1]octan-3-yl)oxy)-2-cyanopyridin-4-yl)pyrazolo[1,5-a]pyridin-2-yl)cyclopropanecarboxamide;
N-(5-(2-cyano-5-(((1R,3R,5S)-8-methyl-8-azabicyclo[3.2.1]octan-3-yl)methoxy)pyridin-4-yl)pyrazolo[1,5-a]pyridin-2-yl)cyclopropanecarboxamide;
N-[5-[2-cyano-5-[[(6S)-1,4-oxazepan-6-yl]oxy]-4-pyridyl]pyrazolo[1,5-a]pyridin-2-yl]cyclopropanecarboxamide;
N-[5-[2-cyano-5-[(1-methyl-4-piperidyl)oxy]-4-pyridyl]pyrazolo[1,5-a]pyridin-2-yl]cyclopropanecarboxamide;
N-(5-(2-cyano-5-(((1s,4s)-4-hydroxycyclohexyl)oxy)pyridin-4-yl)pyrazolo[1,5-a]pyridin-2-yl)cyclopropanecarboxamide;
(rac-)-N-[5-[2-cyano-5-[3-hydroxy-3-(trifluoromethyl)pyrrolidin-1-yl]-4-pyridyl]pyrazolo[1,5-a]pyridin-2-yl]cyclopropanecarboxamide;
(R)—N-(5-(2-cyano-5-((1-methylpiperidin-3-yl)oxy)pyridin-4-yl)pyrazolo[1,5-a]pyridin-2-yl)cyclopropanecarboxamide;
cis-N-(5-(2-cyano-5-(((1S,4S)-4-hydroxy-4-methylcyclohexyl)oxy)pyridin-4-yl)pyrazolo[1,5-a]pyridin-2-yl)cyclopropanecarboxamide;
N-[5-[2-cyano-5-(2-methyl-1,3,3a,4,6,6a-hexahydropyrrolo[3,4-c]pyrrol-5-yl)-4-pyridyl]pyrazolo[1,5-a]pyridin-2-yl]cyclopropanecarboxamide;
N-[5-[2-cyano-5-(2-methyl-2,6-diazaspiro[3.3]heptan-6-yl)-4-pyridyl]pyrazolo[1,5-a]pyridin-2-yl]cyclopropanecarboxamide;
(3-endo)-N-[5-[2-cyano-5-[[(1R,5S)-3-oxa-9-azabicyclo[3.3.1]nonan-7-yl]oxy]-4-pyridyl]pyrazolo[1,5-a]pyridin-2-yl]cyclopropanecarboxamide;
N-[5-[2-cyano-5-[(2,2,6,6-tetramethyl-4-piperidyl)oxy]-4-pyridyl]pyrazolo[1,5-a]pyridin-2-yl]cyclopropanecarboxamide;
N-[5-[2-cyano-5-[(2R)-2-hydroxypropoxy]-4-pyridyl]pyrazolo[1,5-a]pyridin-2-yl]cyclopropanecarboxamide;
N-[5-[2-cyano-5-[[(3S,5R)-5-(trifluoromethyl)-3-piperidyl]oxy]-4-pyridyl]pyrazolo[1,5-a]pyridin-2-yl]cyclopropanecarboxamide;
N-[5-[2-cyano-5-[(2R)-3,3,3-trifluoro-2-hydroxy-propoxy]-4-pyridyl]pyrazolo[1,5-a]pyridin-2-yl]cyclopropanecarboxamide;
N-[5-[2-cyano-5-[(2S)-3,3,3-trifluoro-2-hydroxy-propoxy]-4-pyridyl]pyrazolo[1,5-a]pyridin-2-yl]cyclopropanecarboxamide;
N-(5-(2-cyano-5-(((1s,3s)-3-(methylamino)cyclobutyl)methoxy)pyridin-4-yl)pyrazolo[1,5-a]pyridin-2-yl)cyclopropanecarboxamide;
N-[5-[2-cyano-5-[3,3,3-trifluoro-2-hydroxy-2-(trifluoromethyl)propoxy]-4-pyridyl]pyrazolo[1,5-a]pyridin-2-yl]cyclopropanecarboxamide;
N-[5-[5-cyano-2-[[(3R)-1-methylpyrrolidin-3-yl]methoxy]phenyl]pyrazolo[1,5-a]pyridin-2-yl]cyclopropanecarboxamide;
N-[5-[5-cyano-2-[[(2R)-morpholin-2-yl]methoxy]phenyl]pyrazolo[1,5-a]pyridin-2-yl]cyclopropanecarboxamide;
N-[5-[5-cyano-2-[[(2R)-6,6-dimethylmorpholin-2-yl]methoxy]phenyl]pyrazolo[1,5-a]pyridin-2-yl]cyclopropanecarboxamide;
N-[5-[5-cyano-2-[[(2S)-morpholin-2-yl]methoxy]phenyl]pyrazolo[1,5-a]pyridin-2-yl]cyclopropanecarboxamide;
N-[5-[5-cyano-2-[[(2S)-6,6-dimethylmorpholin-2-yl]methoxy]phenyl]pyrazolo[1,5-a]pyridin-2-yl]cyclopropanecarboxamide;
5-[[(3R)-1-methylpyrrolidin-3-yl]methoxy]-4-[2-(4-pyridylamino)pyrazolo[1,5-a]pyridin-5-yl]pyridine-2-carbonitrile;
4-(2-anilinopyrazolo[1,5-a]pyridin-5-yl)-5-[[(3R)-1-methylpyrrolidin-3-yl]methoxy]pyridine-2-carbonitrile;
5-[[(3R)-1-methylpyrrolidin-3-yl]methoxy]-4-[2-(2-pyridylamino)pyrazolo[1,5-a]pyridin-5-yl]pyridine-2-carbonitrile;
(S)—N-[5-[2-cyano-5-[(3S)-1-(2-methoxyethyl)pyrrolidin-3-yl]oxy-4-pyridyl]pyrazolo[1,5-a]pyridin-2-yl]cyclopropanecarboxamide;
(S)—N-[5-[2-cyano-5-[(3S)-1-(2-pyridylmethyl)pyrrolidin-3-yl]oxy-4-pyridyl]pyrazolo[1,5-a]pyridin-2-yl]cyclopropanecarboxamide;
(R)—N-[5-[2-cyano-5-[(3R)-1-(2,2,2-trifluoroethyl)pyrrolidin-3-yl]oxy-4-pyridyl]pyrazolo[1,5-a]pyridin-2-yl]cyclopropanecarboxamide;
(R)—N-[5-[2-cyano-5-[(3R)-1-(2-methoxyethyl)pyrrolidin-3-yl]oxy-4-pyridyl]pyrazolo[1,5-a]pyridin-2-yl]cyclopropanecarboxamide;
(R)—N-[5-[2-cyano-5-[(3R)-1-(2-pyridylmethyl)pyrrolidin-3-yl]oxy-4-pyridyl]pyrazolo[1,5-a]pyridin-2-yl]cyclopropanecarboxamide;
(R)—N-[5-[2-cyano-5-[(3R)-1-(cyanomethyl)pyrrolidin-3-yl]oxy-4-pyridyl]pyrazolo[1,5-a]pyridin-2-yl]cyclopropanecarboxamide;
(S)—N-[5-[2-cyano-5-[(3S)-1-(cyanomethyl)pyrrolidin-3-yl]oxy-4-pyridyl]pyrazolo[1,5-a]pyridin-2-yl]cyclopropanecarboxamide;
N-(5-(2-methyl-5-((1-methyl azetidin-3-yl)oxy)pyridin-4-yl)pyrazolo[1,5-a]pyridin-2-yl)cyclopropanecarboxamide;
N-(1-(difluoromethyl)-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
5-(5-(((1R,5S,7s)-3-oxa-9-azabicyclo[3.3.1]nonan-7-yl)oxy)-2-methylpyridin-4-yl)-N-(1-(difluoromethyl)-1H-pyrazol-4-yl)pyrazolo[1,5-a]pyridin-2-amine;
N-(4-fluoro-1-methyl-1H-pyrazol-3-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
N-(3-fluoro-1-methyl-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
N-(5-fluoro-1-methyl-1H-pyrazol-3-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
1-(4-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-1H-pyrazol-1-yl)-2-methylpropan-2-ol;
1-(4-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-1-(2-hydroxy-2-methylpropyl)-1H-pyrazol-3-yl)-2,2,2-trifluoroethan-1-one;
7-endo-5-[2-methyl-5-[[(1S,5R)-3-oxa-9-azabicyclo[3.3.1]nonan-7-yl]oxy]-4-pyridyl]-N-(1-methylpyrazol-4-yl)pyrazolo[1,5-a]pyridin-2-amine;
7-endo-N-(1-methyl-1H-pyrazol-4-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
endo-5-(5-(((1R,5S,7s)-3-oxa-9-azabicyclo[3.3.1]nonan-7-yl)oxy)-2-methylpyridin-4-yl)-N-(1-m ethyl-1H-pyrazol-3-yl)pyrazolo[1,5-a]pyridin-2-amine;
7-endo-N-(5-fluoropyridin-2-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
5-(5-(((1R,5S,7s)-3-oxa-9-azabicyclo[3.3.1]nonan-7-yl)oxy)-2-methylpyridin-4-yl)-N-(5-fluoropyridin-2-yl)pyrazolo[1,5-a]pyridin-2-amine;
5-[2-methyl-5-[[(2S)-morpholin-2-yl]methoxy]-4-pyridyl]-N-(1-methylpyrazol-4-yl)pyrazolo[1,5-a]pyridin-2-amine;
5-[2-methyl-5-[[(2S)-morpholin-2-yl]methoxy]-4-pyridyl]-N-(1-methylpyrazol-3-yl)pyrazolo[1,5-a]pyridin-2-amine;
5-[2-methyl-5-[[(2S)-morpholin-2-yl]methoxy]-4-pyridyl]-N-(2-pyridyl)pyrazolo[1,5-a]pyridin-2-amine;
5-[2-methyl-5-[[(2S)-morpholin-2-yl]methoxy]-4-pyridyl]-N-(3-pyridyl)pyrazolo[1,5-a]pyridin-2-amine;
5-[2-methyl-5-[[(2S)-morpholin-2-yl]methoxy]-4-pyridyl]-N-pyrimidin-4-yl-pyrazolo[1,5-a]pyridin-2-amine;
N-(1-isopropylpyrazol-4-yl)-5-[2-methyl-5-[[(2S)-morpholin-2-yl]methoxy]-4-pyridyl]pyrazolo[1,5-a]pyridin-2-amine;
5-[2-methyl-5-[[(2S)-morpholin-2-yl]methoxy]-4-pyridyl]-N-pyridazin-3-yl-pyrazolo[1,5-a]pyridin-2-amine;
N-(1-isopropylpyrazol-3-yl)-5-[2-methyl-5-[[(2S)-morpholin-2-yl]methoxy]-4-pyridyl]pyrazolo[1,5-a]pyridin-2-amine;
endo-N-(5-(5-(((1R,5S,7s)-3-oxa-9-azabicyclo[3.3.1]nonan-7-yl)oxy)-2-methylpyridin-4-yl)pyrazolo[1,5-a]pyridin-2-yl)cyclopropanecarboxamide;
N-[5 [2-methyl-5-[(3R)-1-methylpyrrolidin-3-yl]oxy-4-pyridyl]pyrazolo[1,5-a]pyridin-2-yl]cyclopropanecarboxamide;
N-(5-(2-methyl-5-(((1R,3r,5S)-8-methyl-8-azabicyclo[3.2.1]octan-3-yl)oxy)pyridin-4-yl)pyrazolo[1,5-a]pyridin-2-yl)cyclopropanecarboxamide;
N-[5-[2-methyl-5-[(3S)-1-methylpyrrolidin-3-yl]oxy-4-pyridyl]pyrazolo[1,5-a]pyridin-2-yl]cyclopropanecarboxamide;
N-(5-(2-methyl-5-(((1R,3r,5S)-8-methyl-8-azabicyclo[3.2.1]octan-3-yl)oxy)pyridin-4-yl)pyrazolo[1,5-a]pyridin-2-yl)cyclopropanecarboxamide;
N-[5-[5-(1-ethylazetidin-3-yl)oxy-2-methyl-4-pyridyl]pyrazolo[1,5-a]pyridin-2-yl]cyclopropane carboxamide;
N-[5-[5-(1-isopropylazetidin-3-yl)oxy-2-methyl-4-pyridyl]pyrazolo[1,5-a]pyridin-2-yl]cyclopropanecarboxamide;
N-[5-[2-methyl-5-[[(2S)-morpholin-2-yl]methoxy]-4-pyridyl]pyrazolo[1,5-a]pyridin-2-yl]cyclopropanecarboxamide;
N-[5-[5-[(3-aminooxetan-3-yl)methoxy]-2-methyl-4-pyridyl]pyrazolo[1,5-a]pyridin-2-yl]cyclopropanecarboxamide;
N-[5-[5-[(1-amino-3,3-difluoro-cyclobutyl)methoxy]-2-methyl-4-pyridyl]pyrazolo[1,5-a]pyridin-2-yl]cyclopropanecarboxamide;
trans-N-[5-[5-(3-methoxycyclobutoxy)-2-methyl-4-pyridyl]pyrazolo[1,5-a]pyridin-2-yl]cyclopropanecarboxamide;
cis-N-[5-[5-(3-methoxycyclobutoxy)-2-methyl-4-pyridyl]pyrazolo[1,5-a]pyridin-2-yl]cyclopropanecarboxamide;
cis-N-[5-[5-[(4-hydroxycyclohexyl)methoxy]-2-methyl-4-pyridyl]pyrazolo[1,5-a]pyridin-2-yl]cyclopropanecarboxamide;
N-[5-[5-[(2S)-2-amino-3,3-dimethyl-butoxy]-2-methyl-4-pyridyl]pyrazolo[1,5-a]pyridin-2-yl]cyclopropanecarboxamide;
N-[5-[5-[(2R)-2-amino-3,3-dimethyl-butoxy]-2-methyl-4-pyridyl]pyrazolo[1,5-a]pyridin-2-yl]cyclopropanecarboxamide;
trans-N-(5-(5-(((1r,4r)-4-hydroxy-4-methylcyclohexyl)oxy)-2-methylpyridin-4-yl)pyrazolo[1,5-a]pyridin-2-yl)cyclopropanecarboxamide;
N-[5-[5-[(1R,3S)-3-aminocyclopentoxy]-2-methyl-4-pyridyl]pyrazolo[1,5-a]pyridin-2-yl]cyclopropanecarboxamide;
N-[5-[5-[(1R,3R)-3-aminocyclopentoxy]-2-methyl-4-pyridyl]pyrazolo[1,5-a]pyridin-2-yl]cyclopropanecarboxamide;
N-[5-[5-[(1-aminocyclobutyl)methoxy]-2-methyl-4-pyridyl]pyrazolo[1,5-a]pyridin-2-yl]cyclopropanecarboxamide;
N-[5-[5-[(1-aminocyclopentyl)methoxy]-2-methyl-4-pyridyl]pyrazolo[1,5-a]pyridin-2-yl]cyclopropanecarboxamide;
N-[5-[2-methyl-5-[[1-(methylamino)cyclobutyl]methoxy]-4-pyridyl]pyrazolo[1,5-a]pyridin-2-yl]cyclopropanecarboxamide;
N-[5-[5-[[1-(cyclopropylamino)cyclobutyl]methoxy]-2-methyl-4-pyridyl]pyrazolo[1,5-a]pyridin-2-yl]cyclopropanecarboxamide;
trans-N-[5-[5-(4-hydroxycyclohexoxy)-2-methyl-4-pyridyl]pyrazolo[1,5-a]pyridin-2-yl]cyclopropanecarboxamide;
(*R)—N-[5-[5-(2-amino-3,3,3-trifluoro-2-methyl-propoxy)-2-methyl-4-pyridyl]pyrazolo[1,5-a]pyridin-2-yl]cyclopropanecarboxamide;
(*S)—N-[5-[5-(2-amino-3,3,3-trifluoro-2-methyl-propoxy)-2-methyl-4-pyridyl]pyrazolo[1,5-a]pyridin-2-yl]cyclopropanecarboxamide;
7-endo-N-(5-(5-(((1R,5S,7s)-9-ethyl-3-oxa-9-azabicyclo[3.3.1]nonan-7-yl)oxy)-2-methylpyridin-4-yl)pyrazolo[1,5-a]pyridin-2-yl)cyclopropanecarboxamide;
5-[5-[(1-amino-3,3-difluoro-cyclobutyl)methoxy]-2-methyl-4-pyridyl]-N-(1-methylpyrazol-3-yl) pyrazolo[1,5-a]pyridin-2-amine;
(1r,4r)-1-methyl-4-((6-methyl-4-(2-((1-methyl-1H-pyrazol-4-yl)amino)pyrazolo[1,5-a]pyridin-5-yl)pyridin-3-yl)oxy)cyclohexan-1-ol;
N-(5-(2-methyl-5-(((1S,4S,7R)-5-methyl-2-oxa-5-azabicyclo[2.2.1]heptan-7-yl)oxy)pyridin-4-yl) pyrazolo[1,5-a]pyridin-2-yl)cyclopropanecarboxamide;
N-(5-(5-(((1S,4S,7R)-2-oxa-5-azabicyclo[2.2.1]heptan-7-yl)oxy)-2-methylpyridin-4-yl)pyrazolo[1,5-a]pyridin-2-yl)cyclopropanecarboxamide;
N-[5-[2-methyl-5-[[(2R)-morpholin-2-yl]methoxy]-4-pyridyl]pyrazolo[1,5-a]pyridin-2-yl]cyclopropanecarboxamide;
N-[5-[5-[[(2S)-5,5-dimethylmorpholin-2-yl]methoxy]-2-methyl-4-pyridyl]pyrazolo[1,5-a]pyridin-2-yl]cyclopropanecarboxamide;
N-[5 [2-methyl-5-[[(2S,5R)-5-methylmorpholin-2-yl]methoxy]-4-pyridyl]pyrazolo[1,5-a]pyridin-2-yl]cyclopropanecarboxamide;
N-[5-[5-[(3-fluoro-1-methyl-azetidin-3-yl)methoxy]-2-methyl-4-pyridyl]pyrazolo[1,5-a]pyridin-2-yl]cyclopropanecarboxamide;
trans-N-[5-[5-(3-aminocyclobutoxy)-2-methyl-4-pyridyl]pyrazolo[1,5-a]pyridin-2-yl]cyclopropanecarboxamide;
cis-N-[5-[5-[(3-aminocyclobutyl)methoxy]-2-methyl-4-pyridyl]pyrazolo[1,5-a]pyridin-2-yl]cyclopropanecarboxamide;
2-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-N-methylisonicotinamide;
6-((5-(5-(((1R,5S,7s)-3-oxa-9-azabicyclo[3.3.1]nonan-7-yl)oxy)-2-methylpyridin-4-yl)pyrazolo[1,5-a]pyridin-2-yl)amino)-N-methylnicotinamide;
N-(1-methyl-3-(trifluoromethyl)-1H-pyrazol-5-yl)-5-(2-methyl-5-(((1R,5S,7s)-9-methyl-3-oxa-9-azabicyclo[3.3.1]nonan-7-yl)oxy)pyridin-4-yl)pyrazolo[1,5-a]pyridin-2-amine;
N-(5-(5-(((1s,3s)-3-aminocyclobutyl)methoxy)-2-cyanopyridin-4-yl)pyrazolo[1,5-a]pyridin-2-yl) cyclopropanecarboxamide;
cis-N-[5-[2-cyano-5-[[3-(2-pyridylamino)cyclobutyl]methoxy]-4-pyridyl]pyrazolo[1,5-a]pyridin-2-yl]cyclopropanecarboxamide;
cis-N-[5-[2-cyano-5-[[3-[(1-methylpyrazol-3-yl)amino]cyclobutyl]methoxy]-4-pyridyl]pyrazolo[1,5-a]pyridin-2-yl]cyclopropanecarboxamide;
rac-N-[5-[2-cyano-5-[[4-hydroxy-4-(trifluoromethyl)cyclohexyl]methoxy]-4-pyridyl]pyrazolo[1,5-a]pyridin-2-yl]cyclopropanecarboxamide;
cis-N-(5-(5-(((1*S,3*R)-3-hydroxycyclopentyl)oxy)-2-methylpyridin-4-yl)pyrazolo[1,5-a]pyridin-2-yl)cyclopropanecarboxamide;
cis-N-(5-(5-(((1*R,3*S)-3-hydroxycyclopentyl)oxy)-2-methylpyridin-4-yl)pyrazolo[1,5-a]pyridin-2-yl)cyclopropanecarboxamide;
trans-4-[[6-methyl-4-[2-[(1-methylpyrazol-3-yl)amino]pyrazolo[1,5-a]pyridin-5-yl]-3-pyridyl]oxy]cyclohexanol;
trans-4-[[6-methyl-4-[2-(2-pyridylamino)pyrazolo[1,5-a]pyridin-5-yl]-3-pyridyl]oxy]cyclohexan ol;
trans-N-(5-(5-(((1*S,3*S)-3-hydroxycyclopentyl)oxy)-2-methylpyridin-4-yl)pyrazolo[1,5-a]pyridin-2-yl)cyclopropanecarboxamide;
cis-4-[[6-methyl-4-[2-[(1-methylpyrazol-3-yl)amino]pyrazolo[1,5-a]pyridin-5-yl]-3-pyridyl]oxy]cyclohexanol;
trans-N-[5-[5-(4-hydroxycyclohexoxy)-2-prop-1-ynyl-4-pyridyl]pyrazolo[1,5-a]pyridin-2-yl]cyclopropanecarboxamide;
N-[5-[2-methyl-5-(4-oxocyclohexoxy)-4-pyridyl]pyrazolo[1,5-a]pyridin-2-yl]cyclopropanecarboxamide;
trans-4-[[4-[2-[(1-methylpyrazol-3-yl)amino]pyrazolo[1,5-a]pyridin-5-yl]-6-prop-1-ynyl-3-pyrid yl]oxy]cyclohexanol;
rac-N-[5-[5-[(4,4-difluoro-1-hydroxy-cyclohexyl)methoxy]-2-methyl-4-pyridyl]pyrazolo[1,5-a]pyridin-2-yl]cyclopropanecarboxamide;
(*R)—N-(5-(2-methyl-5-(3,3,3-trifluoro-2-hydroxy-2-methylpropoxy)pyridin-4-yl)pyrazolo[1,5-a]pyridin-2-yl)cyclopropanecarboxamide;
(*S)—N-(5-(2-methyl-5-(3,3,3-trifluoro-2-hydroxy-2-methylpropoxy)pyridin-4-yl)pyrazolo[1,5-a]pyridin-2-yl)cyclopropanecarboxamide;
N-(5-(54(1r,4r)-4-hydroxy-4-(trifluoromethyl)cyclohexyl)oxy)-2-methylpyridin-4-yl)pyrazolo[1,5-a]pyridin-2-yl)cyclopropanecarboxamide;
N-(5-(5-(((1r,1r)-4-hydroxy-4-phenylcyclohexyl)oxy)-2-methylpyridin-4-yl)pyrazolo[1,5-a]pyridin-2-yl)cyclopropanecarboxamide;
(*R)—N-(5-(5-(2-cyclopropyl-2-hydroxypropoxy)-2-methylpyridin-4-yl)pyrazolo[1,5-a]pyridin-2-yl)cyclopropanecarboxamide;
trans-N-(5-(5-(((1*R,3*R)-3-hydroxycyclohexyl)oxy)-2-methylpyridin-4-yl)pyrazolo[1,5-a]pyridin-2-yl)cyclopropanecarboxamide;
trans-N-(5-(5-(((1*S,3*S)-3-hydroxycyclohexyl)oxy)-2-methylpyridin-4-yl)pyrazolo[1,5-a]pyridin-2-yl)cyclopropanecarboxamide;
N-(5-(5-((4-hydroxy-4-isopropylcyclohexyl)oxy)-2-methylpyridin-4-yl)pyrazolo[1,5-a]pyridin-2-yl)cyclopropanecarboxamide;
(*S)—N-(5-(5-(2-cyclohexyl-2-hydroxyethoxy)-2-methylpyridin-4-yl)pyrazolo[1,5-a]pyridin-2-yl) cyclopropanecarboxamide;
trans-4-[[4-[2-[(2,6-dimethylpyrimidin-4-yl)amino]pyrazolo[1,5-a]pyridin-5-yl]-6-methyl-3-pyridyl]oxy]cyclohexanol;
N-[5-[5-(4-hydroxyiminocyclohexoxy)-2-methyl-4-pyridyl]pyrazolo[1,5-a]pyridin-2-yl]cyclopropanecarboxamide;
1-[[4-[2-[(2,6-dimethylpyrimidin-4-yl)amino]pyrazolo[1,5-a]pyridin-5-yl]-6-methyl-3-pyridyl]oxy]-2-methyl-propan-2-ol;
1-[[4-[2-[(5,6-dimethylpyridazin-3-yl)amino]pyrazolo[1,5-a]pyridin-5-yl]-6-methyl-3-pyridyl]oxy]-2-methyl-propan-2-ol;
1-[[4-[2-[(2-ethyl-6-methyl-pyrimidin-4-yl)amino]pyrazolo[1,5-a]pyridin-5-yl]-6-methyl-3-pyrid yl]oxy]-2-methyl-propan-2-ol;
(*R)-3-(((4-(2-((2,6-dimethylpyrimidin-4-yl)amino)pyrazolo[1,5-a]pyridin-5-yl)-6-methylpyridin-3-yl)oxy)methyl)tetrahydrofuran-3-ol;
(3R,5R)-5-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol;
1-[[4-[2-[[2-(methoxymethyl)-6-methyl-pyrimidin-4-yl]amino]pyrazolo[1,5-a]pyridin-5-yl]-6-m ethyl-3-pyridyl]oxy]-2-methyl-propan-2-ol;
N-(2,6-dimethylpyrimidin-4-yl)-5-(2-methyl-5-(((2*S,3*R)-2-methyltetrahydrofuran-3-yl)methoxy)pyridin-4-yl)pyrazolo[1,5-a]pyridin-2-amine;
N-(2,6-dimethylpyrimidin-4-yl)-5-(2-methyl-5-(((2*R,3*S)-2-methyltetrahydrofuran-3-yl)methoxy)pyridin-4-yl)pyrazolo[1,5-a]pyridin-2-amine;
3-[[5-[5-(2-hydroxy-2-methyl-propoxy)-2-methyl-4-pyridyl]pyrazolo[1,5-a]pyridin-2-yl]amino]-5-methyl-1H-pyridazin-6-one;
6-[[5-[5-(2-hydroxy-2-methyl-propoxy)-2-methyl-4-pyridyl]pyrazolo[1,5-a]pyridin-2-yl]amino]-2,4-dimethyl-pyridazin-3-one;
(*S)—N-[5-[5-[(3-cyanopyrrolidin-3-yl)methoxy]-2-methyl-4-pyridyl]pyrazolo[1,5-a]pyridin-2-yl]cyclopropanecarboxamide;
(*R)—N-[5-[5-[[(3R)-3-cyanopyrrolidin-3-yl]methoxy]-2-methyl-4-pyridyl]pyrazolo[1,5-a]pyridin-2-yl]cyclopropanecarboxamide;
(*S)—N-[5-[5-[(3-cyano-1-methyl-pyrrolidin-3-yl)methoxy]-2-methyl-4-pyridyl]pyrazolo[1,5-a]pyridin-2-yl]cyclopropanecarboxamide;
(*R)—N-[5-[5-[(3-cyano-1-methyl-pyrrolidin-3-yl)methoxy]-2-methyl-4-pyridyl]pyrazolo[1,5-a]pyridin-2-yl]cyclopropanecarboxamide;
(*R)—N-[5-[5-[(3-methoxypyrrolidin-3-yl)methoxy]-2-methyl-4-pyridyl]pyrazolo[1,5-a]pyridin-2-yl]cyclopropanecarboxamide;
(*S)—N-[5-[5-[(3-methoxypyrrolidin-3-yl)methoxy]-2-methyl-4-pyridyl]pyrazolo[1,5-a]pyridin-2-yl]cyclopropanecarboxamide;
(*R)-3-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol;
1-[[4-[2-[(2,6-dimethylpyrimidin-4-yl)amino]pyrazolo[1,5-a]pyridin-5-yl]-6-methyl-3-pyridyl]oxymethyl]cyclobutanol;
(2R,3S)-2-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol;
(*S)—N-(2,6-dimethylpyrimidin-4-yl)-5-[2-methyl-5-(8-oxaspiro[4.4]nonan-7-ylmethoxy)-4-pyridyl]pyrazolo[1,5-a]pyridin-2-amine;
(*S)-5-(5-((6-oxaspiro[3.4]octan-7-yl)methoxy)-2-methylpyridin-4-yl)-N-(2,6-dimethylpyrimidin-4-yl)pyrazolo[1,5-a]pyridin-2-amine;
N-[5-[5-[(3-cyanoazetidin-3-yl)methoxy]-2-methyl-4-pyridyl]pyrazolo[1,5-a]pyridin-2-yl]cyclopropanecarboxamide;
(*S)—N-[5-[5-[(3-hydroxypyrrolidin-3-yl)methoxy]-2-methyl-4-pyridyl]pyrazolo[1,5-a]pyridin-2-yl]-cyclopropanecarboxamide;
(*R)—N-[5-[5-[(3-hydroxypyrrolidin-3-yl)methoxy]-2-methyl-4-pyridyl]pyrazolo[1,5-a]pyridin-2-yl]cyclopropanecarboxamide;
(1r,4r)-4-((4-(2-((2-ethyl-6-methylpyrimidin-4-yl)amino)pyrazolo[1,5-a]pyridin-5-yl)-6-methylpyridin-3-yl)oxy)cyclohexan-1-ol;
(1r,4r)-4-((4-(2-((2-(methoxymethyl)-6-methylpyrimidin-4-yl)amino)pyrazolo[1,5-a]pyridin-5-yl)-6-methylpyridin-3-yl)oxy)cyclohexan-1-ol;
6-((5-(5-(((1r,4r)-4-hydroxycyclohexyl)oxy)-2-methylpyridin-4-yl)pyrazolo[1,5-a]pyridin-2-yl)amino)-2,4-dimethylpyridazin-3 (2H)-one;
(1r,4r)-4-((6-methyl-4-(2-((6-methyl-2-(methylamino)pyrimidin-4-yl)amino)pyrazolo[1,5-a]pyridin-5-yl)pyridin-3-yl)oxy)cyclohexan-1-ol;
2-methyl-1-[[6-methyl-4-[2-[[6-methyl-2-(methylamino)pyrimidin-4-yl]amino]pyrazolo[1,5-a]pyridin-5-yl]-3-pyridyl]oxy]propan-2-ol;
1-[[4-[2-[(2-cyclopropyl-6-methyl-pyrimidin-4-yl)amino]pyrazolo[1,5-a]pyridin-5-yl]-6-methyl-3-pyridyl]oxy]-2-methyl-propan-2-ol;
(*S)—N-(2,6-dimethylpyrimidin-4-yl)-5-[2-methyl-5-(9-oxaspiro[4.4]nonan-8-ylmethoxy)-4-pyridyl]pyrazolo[1,5-a]pyridin-2-amine;
(*R)—N-(2,6-dimethylpyrimidin-4-yl)-5-[2-methyl-5-(9-oxaspiro[4.4]nonan-8-ylmethoxy)-4-pyridyl]pyrazolo[1,5-a]pyridin-2-amine;
(*S)—N-(2,6-dimethylpyrimidin-4-yl)-5-[2-methyl-5-[(2-methyltetrahydrofuran-2-yl)methoxy]-4-pyridyl]pyrazolo[1,5-a]pyridin-2-amine
N-[5-[5-[(3-methoxyazetidin-3-yl)methoxy]-2-methyl-4-pyridyl]pyrazolo[1,5-a]pyridin-2-yl]cyclopropanecarboxamide;
(1R,2R)-2-methyl-N-[5-[2-methyl-5-[[(1R,4R)-5-oxa-2-azabicyclo[2.2.1]heptan-4-yl]methoxy]-4-pyridyl]pyrazolo[1,5-a]pyridin-2-yl]cyclopropanecarboxamide;
N-(6-cyclopropylpyridazin-3-yl)-5-[2-methyl-5-[[(1R,4R)-2-methyl-5-oxa-2-azabicyclo[2.2.1]heptan-4-yl]methoxy]-4-pyridyl]pyrazolo[1,5-a]pyridin-2-amine;
5-[2-methyl-5-[[(1R,4R)-2-methyl-5-oxa-2-azabicyclo[2.2.1]heptan-4-yl]methoxy]-4-pyridyl]-N-(5-methylpyrazin-2-yl)pyrazolo[1,5-a]pyridin-2-amine;
N-[5-[2-methyl-5-[[(1S,4S)-2-methyl-5-oxa-2-azabicyclo[2.2.1]heptan-4-yl]methoxy]-4-pyridyl]pyrazolo[1,5-a]pyridin-2-yl]cyclopropanecarboxamide;
N-(2,6-dimethylpyrimidin-4-yl)-5-[2-methyl-5-[[(1R,4R)-5-oxa-2-azabicyclo[2.2.1]heptan-4-yl]methoxy]-4-pyridyl]pyrazolo[1,5-a]pyridin-2-amine;
4-[[6-methyl-4-[2-(1H-pyrrolo[2,3-b]pyridin-6-ylamino)pyrazolo[1,5-a]pyridin-5-yl]-3-pyridyl]oxy]cyclohexanol;
2-methyl-1-[[6-methyl-4-[2-(1H-pyrrolo[2,3-b]pyridin-6-ylamino)pyrazolo[1,5-a]pyridin-5-yl]-3-pyridyl]oxy]propan-2-ol;
4-[[5-[5-(4-hydroxycyclohexoxy)-2-methyl-4-pyridyl]pyrazolo[1,5-a]pyridin-2-yl]amino]-2,6-di methoxy-N-(2,2,2-trifluoroethyl)benzamide;
N-cyclopropyl-4-[[5-[5-(4-hydroxycyclohexoxy)-2-methyl-4-pyridyl]pyrazolo[1,5-a]pyridin-2-yl]amino]-2-methoxy-benzamide;
N-ethyl-4-[[5-[5-(4-hydroxycyclohexoxy)-2-methyl-4-pyridyl]pyrazolo[1,5-a]pyridin-2-yl]amino]-2,6-dimethoxy-benzamide;
6-[[5-[5-(2-hydroxy-2-methyl-propoxy)-2-methyl-4-pyridyl]pyrazolo[1,5-a]pyridin-2-yl]amino]-N,N,4-trimethyl-pyridine-2-carboxamide;
N-cyclopropyl-4-[[5-[5-(4-hydroxycyclohexoxy)-2-methyl-4-pyridyl]pyrazolo[1,5-a]pyridin-2-yl]amino]-2,6-dimethoxy-benzamide;
methyl 4-[[5-[5-(4-hydroxycyclohexoxy)-2-methyl-4-pyridyl]pyrazolo[1,5-a]pyridin-2-yl]amino]-2-methoxy-benzoate;
2-ethyl-6-[[5-[5-(2-hydroxy-2-methyl-propoxy)-2-methyl-4-pyridyl]pyrazolo[1,5-a]pyridin-2-yl]amino]-4-methyl-pyridazin-3-one;
N-[5-[2-methyl-5-[[(1R,4R)-5-oxa-2-azabicyclo[2.2.1]heptan-4-yl]methoxy]-4-pyridyl]pyrazolo[1,5-a]pyridin-2-yl]cyclopropanecarboxamide;
N-[5-[2-methyl-5-[[(1R,4R)-2-methyl-5-oxa-2-azabicyclo[2.2.1]heptan-4-yl]methoxy]-4-pyridyl]pyrazolo[1,5-a]pyridin-2-yl]cyclopropanecarboxamide;
(1R,2R)-2-methyl-N-[5-[2-methyl-5-[[(1R,4R)-2-methyl-5-oxa-2-azabicyclo[2.2.1]heptan-4-yl]methoxy]-4-pyridyl]pyrazolo[1,5-a]pyridin-2-yl]cyclopropanecarboxamide;
5-[2-methyl-5-[[(1S,4S)-5-oxa-2-azabicyclo[2.2.1]heptan-4-yl]methoxy]-4-pyridyl]-N-(5-methyl pyrazin-2-yl)pyrazolo[1,5-a]pyridin-2-amine;
N-(2,6-dimethylpyrimidin-4-yl)-5-[5-[[(1R,4R)-5-oxa-2-azabicyclo[2.2.1]heptan-4-yl]methoxy]-2-(trifluoromethyl)-4-pyridyl]pyrazolo[1,5-a]pyridin-2-amine;
N-[5-[2-methyl-5-[[(1S,4S)-5-oxa-2-azabicyclo[2.2.1]heptan-4-yl]methoxy]-4-pyridyl]pyrazolo[1,5-a]pyridin-2-yl]cyclopropanecarboxamide;
N-(2,6-dimethylpyrimidin-4-yl)-5-[5-[[(1R,4R)-5-oxa-2-azabicyclo[2.2.1]heptan-4-yl]methoxy]-2-(trifluoromethyl)-4-pyridyl]pyrazolo[1,5-a]pyridin-2-amine;
N-(6-cyclopropylpyridazin-3-yl)-5-[2-methyl-5-[[(1R,4R)-5-oxa-2-azabicyclo[2.2.1]heptan-4-yl]methoxy]-4-pyridyl]pyrazolo[1,5-a]pyridin-2-amine;
5-[2-methyl-5-[[(1R,4R)-2-methyl-5-oxa-2-azabicyclo[2.2.1]heptan-4-yl]methoxy]-4-pyridyl]-N-(6-methylpyridazin-3-yl)pyrazolo[1,5-a]pyridin-2-amine;
5-[2-methyl-5-[[(1R,4R)-5-oxa-2-azabicyclo[2.2.1]heptan-4-yl]methoxy]-4-pyridyl]-N-(5-methylpyrazin-2-yl)pyrazolo[1,5-a]pyridin-2-amine;
(*R)—N-(2,6-dimethylpyrimidin-4-yl)-5-[2-methyl-5-(8-oxaspiro[4.4]nonan-7-ylmethoxy)-4-pyridyl]pyrazolo[1,5-a]pyridin-2-amine;
2-[[4-[2-[(2,6-dimethylpyrimidin-4-yl)amino]pyrazolo[1,5-a]pyridin-5-yl]-6-methyl-3-pyridyl]oxymethyl]-1,1,1,3,3,3-hexafluoro-propan-2-ol 1-[[4-[2-(imidazo[1,2-b]pyridazin-6-ylamino)pyrazolo[1,5-a]pyridin-5-yl]-6-methyl-3-pyridyl]oxy]-2-methyl-propan-2-ol;
1-[[4-[2-[(2-tert-butyl-6-methyl-pyrimidin-4-yl)amino]pyrazolo[1,5-a]pyridin-5-yl]-6-methyl-3-pyridyl]oxy]-2-methyl-propan-2-ol;
3-[[4-[2-[(2,6-dimethylpyrimidin-4-yl)amino]pyrazolo[1,5-a]pyridin-5-yl]-6-methyl-3-pyridyl]oxy]-2,2-dimethyl-propanenitrile;
(*S)-3-[[4-[2-[(2,6-dimethylpyrimidin-4-yl)amino]pyrazolo[1,5-a]pyridin-5-yl]-6-methyl-3-pyrid yl]oxy]-1,1,1-trifluoro-2-methyl-propan-2-ol;
(*R)-3-[[4-[2-[(2,6-dimethylpyrimidin-4-yl)amino]pyrazolo[1,5-a]pyridin-5-yl]-6-methyl-3-pyridyl]oxy]-1,1,1-trifluoro-2-methyl-propan-2-ol;
2-(cyclopropylmethyl)-6-[[5-[5-(2-hydroxy-2-methyl-propoxy)-2-methyl-4-pyridyl]pyrazolo[1,5-a]pyridin-2-yl]amino]-4-methyl-pyridazin-3-one;
N-(2,6-dimethylpyrimidin-4-yl)-5-[2-methyl-5-(4-methylsulfonylcyclohexoxy)-4-pyridyl]pyrazolo[1,5-a]pyridin-2-amine;
(rac-)-5-[5-(2-amino-3,3,3-trifluoro-2-methyl-propoxy)-2-methyl-4-pyridyl]-N-(2,6-dimethylpyrimidin-4-yl)pyrazolo[1,5-a]pyridin-2-amine;
4-[[5-[5-(2-cyano-2-methyl-propoxy)-2-methyl-4-pyridyl]pyrazolo[1,5-a]pyridin-2-yl]amino]-N-cyclopropyl-2,6-dimethoxy-benzamide;
3-[[4-[2-[(1,5-dimethyl-6-oxo-pyridazin-3-yl)amino]pyrazolo[1,5-a]pyridin-5-yl]-6-methyl-3-pyridyl]oxy]-2,2-dimethyl-propanenitrile;
(*R)-5-[5-(2-Amino-3,3,3-trifluoro-2-methyl-propoxy)-2-methyl-4-pyridyl]-N-(2,6-dimethylpyrimidin-4-yl)pyrazolo[1,5-a]pyridin-2-amine;
N-(2,6-dimethylpyrimidin-4-yl)-5-[5-[(2-methoxyphenyl)methoxy]-2-methyl-4-pyridyl]pyrazolo[1,5-a]pyridin-2-amine;
3-[[4-[2-[(2,6-dimethylpyrimidin-4-yl)amino]pyrazolo[1,5-a]pyridin-5-yl]-6-methyl-3-pyridyl]oxymethyl]phenol;
N-(2,6-dimethylpyrimidin-4-yl)-5-[5-[(3-ethyloxetan-3-yl)methoxy]-2-methyl-4-pyridyl]pyrazolo[1,5-a]pyridin-2-amine;
[3-[[4-[2-[(2,6-dimethylpyrimidin-4-yl)amino]pyrazolo[1,5-a]pyridin-5-yl]-6-methyl-3-pyridyl]oxymethyl]oxetan-3-yl]methanol;
3-[[4-[2-[(2,6-dimethylpyrimidin-4-yl)amino]pyrazolo[1,5-a]pyridin-5-yl]-6-methyl-3-pyridyl]oxymethyl]oxetane-3-carbonitrile;
N-(2,6-dimethylpyrimidin-4-yl)-5-[2-methyl-5-[(3S)-pyrrolidin-3-yl]oxy-4-pyridyl]pyrazolo[1,5-a]pyridin-2-amine;
N-[5-[2-methyl-5-[(3S)-pyrrolidin-3-yl]oxy-4-pyridyl]pyrazolo[1,5-a]pyridin-2-yl]cyclopropane carboxamide;
N-(2,6-dimethylpyrimidin-4-yl)-5-[5-[(3R,4S)-4-fluoropyrrolidin-3-yl]oxy-2-methyl-4-pyridyl]pyrazolo[1,5-a]pyridin-2-amine;
N-[5-[5-[(3R,4S)-4-fluoropyrrolidin-3-yl]oxy-2-methyl-4-pyridyl]pyrazolo[1,5-a]pyridin-2-yl]cyclopropanecarboxamide;
N-[5-[2-prop-1-ynyl-5-[(3S)-pyrrolidin-3-yl]oxy-4-pyridyl]pyrazolo[1,5-a]pyridin-2-yl]cyclopropanecarboxamide;
N-[5-[5-[(3R,4S)-4-fluoropyrrolidin-3-yl]oxy-2-prop-1-ynyl-4-pyridyl]pyrazolo[1,5-a]pyridin-2-yl]cyclopropanecarboxamide;
6-[[5-[5-(2-hydroxy-2-methyl-propoxy)-2-methyl-4-pyridyl]pyrazolo[1,5-a]pyridin-2-yl]amino]-2-isobutyl-4-methyl-pyridazin-3-one;
2-[2-(difluoromethoxy)ethyl]-6-[[5-[5-(4-hydroxycyclohexoxy)-2-methyl-4-pyridyl]pyrazolo[1,5-a]pyridin-2-yl]amino]-4-methyl-pyridazin-3-one;
2-[2-(difluoromethoxy)ethyl]-6-[[5-[5-(2-hydroxy-2-methyl-propoxy)-2-methyl-4-pyridyl]pyrazolo[1,5-a]pyridin-2-yl]amino]-4-methyl-pyridazin-3-one;
2-methyl-1-[[6-methyl-4-[2-[(6-methyl-2-pyrrolidin-1-yl-pyrimidin-4-yl)amino]pyrazolo[1,5-a]pyridin-5-yl]-3-pyridyl]oxy]propan-2-ol;
2-methyl-1-[[6-methyl-4-[2-[(6-methyl-2-morpholino-pyrimidin-4-yl)amino]pyrazolo[1,5-a]pyridin-5-yl]-3-pyridyl]oxy]propan-2-ol;
4-[[6-methyl-4-[2-[(6-methyl-2-pyrrolidin-1-yl-pyrimidin-4-yl)amino]pyrazolo[1,5-a]pyridin-5-y 1]-3-pyridyl]oxy]cyclohexanol;
4-[[6-methyl-4-[2-[(6-methyl-2-morpholino-pyrimidin-4-yl)amino]pyrazolo[1,5-a]pyridin-5-yl]-3-pyridyl]oxy]cyclohexanol;
4-[[6-methyl-4-[2-(5H-pyrrolo[2,3-b]pyrazin-3-ylamino)pyrazolo[1,5-a]pyridin-5-yl]-3-pyridyl]oxy]cyclohexanol;
(1r,4r)-4-((6-methyl-4-(2-(pyridin-3-ylamino)pyrazolo[1,5-a]pyridin-5-yl)pyridin-3-yl)oxy)cyclo hexan-1-ol;
2-methyl-1-[[6-methyl-4-[2-(5H-pyrrolo[2,3-b]pyrazin-3-ylamino)pyrazolo[1,5-a]pyridin-5-yl]-3-pyridyl]oxy]propan-2-ol;
2-methyl-1-[[6-methyl-4-[2-[(1-methylpyrazolo[3,4-b]pyridin-3-yl)amino]pyrazolo[1,5-a]pyridin-5-yl]-3-pyridyl]oxy]propan-2-ol;
2-ethyl-6-[[5-[5-[[(2R,3S)-3-hydroxytetrahydrofuran-2-yl]methoxy]-2-methyl-4-pyridyl]pyrazolo[1,5-a]pyridin-2-yl]amino]-4-methyl-pyridazin-3-one;
(2R,3S)-2-[[6-methyl-4-[2-[(6-methyl-2-morpholino-pyrimidin-4-yl)amino]pyrazolo[1,5-a]pyridin-5-yl]-3-pyridyl]oxymethyl]tetrahydrofuran-3-ol;
(2R,3S)-2-[[4-[2-[[5-(difluoromethyl)pyrazin-2-yl]amino]pyrazolo[1,5-a]pyridin-5-yl]-6-methyl-3-pyridyl]oxymethyl]tetrahydrofuran-3-ol;
2-[2-(difluoromethoxy)ethyl]-6-[[5-[5-[[(2R,3S)-3-hydroxytetrahydrofuran-2-yl]methoxy]-2-methyl-4-pyridyl]pyrazolo[1,5-a]pyridin-2-yl]amino]-4-methyl-pyridazin-3-one;
(2R,3S)-2-[[6-methyl-4-[2-[(6-methylpyrazin-2-yl)amino]pyrazolo[1,5-a]pyridin-5-yl]-3-pyridyl]oxymethyl]tetrahydrofuran-3-ol;
(2R,3S)-2-[[4-[2-[(2-tert-butyl-6-methyl-pyrimidin-4-yl)amino]pyrazolo[1,5-a]pyridin-5-yl]-6-m ethyl-3-pyridyl]oxymethyl]tetrahydrofuran-3-ol;
(2R,3S)-2-[[6-methyl-4-[2-[(1-methylindazol-3-yl)amino]pyrazolo[1,5-a]pyridin-5-yl]-3-pyridyl]oxymethyl]tetrahydrofuran-3-ol;
(2R,3S)-2-[[4-[2-[(1-methylpyrazol-4-yl)amino]pyrazolo[1,5-a]pyridin-5-yl]-6-prop-1-ynyl-3-pyridyl]oxymethyl]tetrahydrofuran-3-ol;
(2R,3S)-2-[[4-[2-[(1-methylpyrazol-3-yl)amino]pyrazolo[1,5-a]pyridin-5-yl]-6-prop-1-ynyl-3-pyridyl]oxymethyl]tetrahydrofuran-3-ol;
N-(2,6-dimethylpyrimidin-4-yl)-5-[5-[[(1S,4S)-5-oxa-2-azabicyclo[2.2.1]heptan-4-yl]methoxy]-2-prop-1-ynyl-4-pyridyl]pyrazolo[1,5-a]pyridin-2-amine;
5-[2-chloro-5-[[(1S,4S)-5-oxa-2-azabicyclo[2.2.1]heptan-4-yl]methoxy]-4-pyridyl]-N-(2,6-dimethylpyrimidin-4-yl)pyrazolo[1,5-a]pyridin-2-amine;
(*S)-5-[5-(2-amino-3,3,3-trifluoro-2-methyl-propoxy)-2-methyl-4-pyridyl]-N-(2,6-dimethylpyrimidin-4-yl)pyrazolo[1,5-a]pyridin-2-amine;
2,4-dimethyl-6-((5-(2-methyl-5-(((1s,4s)-4-(methylsulfonyl)cyclohexyl)oxy)pyridin-4-yl)pyrazolo[1,5-a]pyridin-2-yl)amino)pyridazin-3 (2H)-one;
2,4-dimethyl-6-((5-(2-methyl-5-(((1r,4r)-4-(methylsulfonyl)cyclohexyl)oxy)pyridin-4-yl)pyrazolo[1,5-a]pyridin-2-yl)amino)pyridazin-3(2H)-one;
2,2-dimethyl-3-[[6-methyl-4-[2-(1H-pyrrolo[2,3-b]pyridin-6-ylamino)pyrazolo[1,5-a]pyridin-5-y 1]-3-pyridyl]oxy]propanenitrile;
3-[[4-[2-(imidazo[1,2-b]pyridazin-6-ylamino)pyrazolo[1,5-a]pyridin-5-yl]-6-methyl-3-pyridyl]oxy]-2,2-dimethyl-propanenitrile;
2,2-dimethyl-3[[6-methyl-4-[2-[[6-methyl-2-(methylamino)pyrimidin-4-yl]amino]pyrazolo[1,5-a]pyridin-5-yl]-3-pyridyl]oxy]propanenitrile;
3-[[4[2-[(2-cyclopropyl-6-methyl-pyrimidin-4-yl)amino]pyrazolo[1,5-a]pyridin-5-yl]-6-methyl-3-pyridyl]oxy]-2,2-dimethyl-propanenitrile;
(*R)-6-[[5-[5-(2-amino-3,3,3-trifluoro-2-methyl-propoxy)-2-methyl-4-pyridyl]pyrazolo[1,5-a]pyridin-2-yl]amino]-2,4-dimethyl-pyridazin-3-one;
(*S)-6-[[5-[5-(2-amino-3,3,3-trifluoro-2-methyl-propoxy)-2-methyl-4-pyridyl]pyrazolo[1,5-a]pyridin-2-yl]amino]-2,4-dimethyl-pyridazin-3-one;
1-[[4-[2-[(2,6-dimethylpyrimidin-4-yl)amino]pyrazolo[1,5-a]pyridin-5-yl]-6-methyl-3-pyridyl]oxymethyl]cyclopropanecarboxamide;
5-[5-(3-aminocyclobutoxy)-2-methyl-4-pyridyl]-N-(2,6-dimethylpyrimidin-4-yl)pyrazolo[1,5-a]pyridin-2-amine;
4-[[4-[2-[(2,6-dimethylpyrimidin-4-yl)amino]pyrazolo[1,5-a]pyridin-5-yl]-6-methyl-3-pyridyl]oxy]-2,2-dimethyl-butanenitrile;
3-[[4-[2-[(1-cyclopropyl-5-methyl-6-oxo-pyridazin-3-yl)amino]pyrazolo[1,5-a]pyridin-5-yl]-6-m ethyl-3-pyridyl]oxy]-2,2-dimethyl-propanenitrile;
2-cyclopropyl-6-[[5-[5-[[(2R,3S)-3-hydroxytetrahydrofuran-2-yl]methoxy]-2-methyl-4-pyridyl]pyrazolo[1,5-a]pyridin-2-yl]amino]-4-methyl-pyridazin-3-one;
2-cyclopropyl-6-[[5-[5-(4-hydroxycyclohexoxy)-2-methyl-4-pyridyl]pyrazolo[1,5-a]pyridin-2-yl]amino]-4-methyl-pyridazin-3-one;
N-[5-[2-methyl-5-[[(1S,5R)-9-methyl-3-oxa-9-azabicyclo[3.3.1]nonan-7-yl]oxy]-4-pyridyl]pyrazolo[1,5-c]pyrimidin-2-yl]cyclopropanecarboxamide;
6-[2-methyl-5-[[(1S,5R)-3-oxa-9-azabicyclo[3.3.1]nonan-7-yl]oxy]-4-pyridyl]-N-(5-methylpyrazin-2-yl)imidazo[1,2-a]pyrazin-2-amine;
6-[2-methyl-5-[[(1S,5R)-3-oxa-9-azabicyclo[3.3.1]nonan-7-yl]oxy]-4-pyridyl]-N-(6-methylpyrazin-2-yl)imidazo[1,2-a]pyrazin-2-amine;
6-[2-methyl-5-[[(1S,5R)-3-oxa-9-azabicyclo[3.3.1]nonan-7-yl]oxy]-4-pyridyl]-N-(1-methylpyrazol-4-yl)imidazo[1,2-a]pyrazin-2-amine;
N-cyclopropyl-4-[[5-[5-(4-hydroxycyclohexoxy)-2-methyl-4-pyridyl]pyrazolo[1,5-a]pyridin-2-yl]amino]benzenesulfonamide;
4-[[5-[5-(4-hydroxycyclohexoxy)-2-methyl-4-pyridyl]pyrazolo[1,5-a]pyridin-2-yl]amino]-N,N-dimethyl-benzenesulfonamide;
rac-4-[[4-[2-[(2,6-dimethylpyrimidin-4-yl)amino]pyrazolo[1,5-a]pyridin-5-yl]-6-methyl-3-pyrid yl]oxymethyl]-1,5,5-trimethyl-pyrrolidin-2-one;
N-cyclopropyl-4-[[5-[5-(2-hydroxy-2-methyl-propoxy)-2-methyl-4-pyridyl]pyrazolo[1,5-a]pyridin-2-yl]amino]-2,6-dimethyl-benzamide;
1-[[4-[2-[(2,6-dimethylpyrimidin-4-yl)amino]pyrazolo[1,5-a]pyridin-5-yl]-6-(fluoromethyl)-3-pyridyl]oxy]-2-methyl-propan-2-ol;
3-[[4-[2-[(2,6-dimethylpyrimidin-4-yl)amino]pyrazolo[1,5-a]pyridin-5-yl]-6-ethyl-3-pyridyl]oxy]-2,2-dimethyl-propanenitrile;
3-[[6-(difluoromethoxy)-4-[2-[(2,6-dimethylpyrimidin-4-yl)amino]pyrazolo[1,5-a]pyridin-5-yl]-3-pyridyl]oxy]-2,2-dimethyl-propanenitrile;
4-[[5-[5-(4-hydroxycyclohexoxy)-2-methyl-4-pyridyl]pyrazolo[1,5-a]pyridin-2-yl]amino]-N-isopropyl-2,6-dimethoxy-benzamide;
4-[[5-[5-(4-hydroxycyclohexoxy)-2-methyl-4-pyridyl]pyrazolo[1,5-a]pyridin-2-yl]amino]-2,6-dimethoxy-N-methyl-benzamide;
4-[[5-[5-(4-hydroxycyclohexoxy)-2-methyl-4-pyridyl]pyrazolo[1,5-a]pyridin-2-yl]amino]-2,6-dimethoxy-N,N-dimethyl-benzamide;
N-cyclopropyl-2-(difluoromethoxy)-4-[[5-[5-(4-hydroxycyclohexoxy)-2-methyl-4-pyridyl]pyrazolo[1,5-a]pyridin-2-yl]amino]-6-methoxy-benzamide;
N-cyclopropyl-6-[[5-[5-(4-hydroxycyclohexoxy)-2-methyl-4-pyridyl]pyrazolo[1,5-a]pyridin-2-yl]amino]-2,4-dimethyl-pyridine-3-carboxamide;
N-cyclopropyl-6-[[5-[5-(2-hydroxy-2-methyl-propoxy)-2-methyl-4-pyridyl]pyrazolo[1,5-a]pyridin-2-yl]amino]-2,4-dimethyl-pyridine-3-carboxamide;
(1S,5R)-7-[[4-[2-[(2,6-dimethylpyrimidin-4-yl)amino]pyrazolo[1,5-a]pyridin-5-yl]-6-methyl-3-pyridyl]oxy]-3-oxa-9-azabicyclo[3.3.1]nonane-9-carbonitrile;
2,4-dimethyl-6-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]pyridazin-3-one;
N-(3-fluoro-4-methyl-2-pyridyl)-5-[2-methyl-5-[[(1S,5R)-3-oxa-9-azabicyclo[3.3.1]nonan-7-yl]oxy]-4-pyridyl]pyrazolo[1,5-a]pyridin-2-amine;
N-[5-[5-[[(1S,5R)-9-acetyl-3-oxa-9-azabicyclo[3.3.1]nonan-7-yl]oxy]-2-methyl-4-pyridyl]pyrazolo[1,5-a]pyridin-2-yl]cyclopropanecarboxamide;
N-isopropyl-2,6-dimethoxy-4-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]benzamide;
N-isopropyl-2-methyl-5-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]pyrazole-3-carboxamide
(4R)-4-[[4-[2-[(2,6-dimethylpyrimidin-4-yl)amino]pyrazolo[1,5-a]pyridin-5-yl]-6-methyl-3-pyridyl]oxymethyl]oxazolidin-2-one;
4-[[4-[2-[(2,6-dimethylpyrimidin-4-yl)amino]pyrazolo[1,5-a]pyridin-5-yl]-6-methyl-3-pyridyl]oxy]cyclohexanecarboxamide;
N-(2,6-dimethylpyrimidin-4-yl)-5-[5-[(3R)-1,1-dioxothiolan-3-yl]oxy-2-methyl-4-pyridyl]pyrazolo[1,5-a]pyridin-2-amine;
4-[[5-[5-(2-hydroxy-2-methyl-propoxy)-2-methyl-4-pyridyl]pyrazolo[1,5-a]pyridin-2-yl]amino]-N-isopropyl-2-methyl-benzamide;
(3*R,6*S)-6-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol;
(3*S,6*S)-6-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol;
N-(2,6-dimethylpyrimidin-4-yl)-6-[2-methyl-5-[[(1R,5S)-3-oxa-9-azabicyclo[3.3.1]nonan-7-yl]oxy]-4-pyridyl]imidazo[1,2-a]pyrazin-2-amine;
N-(2,6-dimethylpyrimidin-4-yl)-6-[2-methyl-5-[[(1R,5S)-9-methyl-3-oxa-9-azabicyclo[3.3.1]nonan-7-yl]oxy]-4-pyridyl]imidazo[1,2-a]pyrazin-2-amine;
(3*R, 5*S)-5-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol;
(RS)—N-(2,6-dimethylpyrimidin-4-yl)-5-[5-[(1,1-dioxo-1,2,5-thiadiazolidin-3-yl)methoxy]-2-methyl-4-pyridyl]pyrazolo[1,5-a]pyridin-2-amine;
(RS)—N-(2,6-dimethylpyrimidin-4-yl)-5-[5-[(1,1-dioxo-1,2-thiazolidin-3-yl)methoxy]-2-methyl-4-pyridyl]pyrazolo[1,5-a]pyridin-2-amine;
(*R)—N-(2,6-dimethylpyrimidin-4-yl)-5-[2-methyl-5-(2-methyl-1,1-dioxo-thiazinan-4-yl)oxy-4-pyridyl]pyrazolo[1,5-a]pyridin-2-amine;
(*S)—N-(2,6-dimethylpyrimidin-4-yl)-5-[2-methyl-5-(2-methyl-1,1-dioxo-thiazinan-4-yl)oxy-4-pyridyl]pyrazolo[1,5-a]pyridin-2-amine;
6-[[5-[5-[[(1S,5R)-9-acetyl-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide;
2,6-dimethoxy-N-methyl-4-[[5-[2-methyl-5-[[(1S,5R)-3-oxa-9-azabicyclo[3.3.1]nonan-7-yl]oxy]-4-pyridyl]pyrazolo[1,5-a]pyridin-2-yl]amino]benzamide;
6-[[5-[5-[[(1R,5S,7s)-9-acetyl-3-oxa-9-azabicyclo[3.3.1]nonan-7-yl]oxy]-2-methyl-4-pyridyl]pyrazolo[1,5-a]pyridin-2-yl]amino]-N,2,4-trimethyl-pyridine-3-carboxamide;
5-[5-(2-amino-2-methyl-propoxy)-2-methyl-4-pyridyl]-N-(2,6-dimethylpyrimidin-4-yl)pyrazolo[1,5-a]pyridin-2-amine;
N-[2-[[4-[2-[(2,6-dimethylpyrimidin-4-yl)amino]pyrazolo[1,5-a]pyridin-5-yl]-6-methyl-3-pyridyl]oxy]-1,1-dimethyl-ethyl]acetamide;
N-[2-[[4-[2-[(2,6-dimethylpyrimidin-4-yl)amino]pyrazolo[1,5-a]pyridin-5-yl]-6-methyl-3-pyridyl]oxy]-1,1-dimethyl-ethyl]methanesulfonamide;
4-[[4-[2-[(2,6-dimethylpyrimidin-4-yl)amino]pyrazolo[1,5-a]pyridin-5-yl]-6-prop-1-ynyl-3-pyrid yl]amino]cyclohexanol;
(*R)—N-(2,6-dimethylpyrimidin-4-yl)-5-[5-[(1,1-dioxothiolan-3-yl)methoxy]-2-methyl-4-pyridyl]pyrazolo[1,5-a]pyridin-2-amine;
(*S)—N-(2,6-dimethylpyrimidin-4-yl)-5-[5-[(1,1-dioxothiolan-3-yl)methoxy]-2-methyl-4-pyridyl]pyrazolo[1,5-a]pyridin-2-amine;
(4S)-4-[[4-[2-[(2,6-dimethylpyrimidin-4-yl)amino]pyrazolo[1,5-a]pyridin-5-yl]-6-methyl-3-pyrid yl]oxymethyl]-1-methyl-imidazolidin-2-one;
5-[2-methyl-5-[[(1S,5R,7s)-3-oxa-9-azabicyclo[3.3.1]nonan-7-yl]oxy]-4-pyridyl]-N-[6-(trifluoromethyl)-2-pyridyl]pyrazolo[1,5-a]pyridin-2-amine;
N-methyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-2-carboxamide;
5-[2-methyl-5-[[(1S,5R,7s)-3-oxa-9-azabicyclo[3.3.1]nonan-7-yl]oxy]-4-pyridyl]-N-pyridazin-4-yl-pyrazolo[1,5-a]pyridin-2-amine;
N-(2-hydroxyethyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxylic acid;
N-(2-hydroxy-2-methyl-propyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
5-[2-methyl-5-[[(1S,5R,7s)-3-oxa-9-azabicyclo[3.3.1]nonan-7-yl]oxy]-4-pyridyl]-N-(2-methylpyrimidin-4-yl)pyrazolo[1,5-a]pyridin-2-amine;
6-[[5-[2-methyl-5-[[(1R,5S,7s)-3-oxa-9-azabicyclo[3.3.1]nonan-7-yl]oxy]-4-pyridyl]pyrazolo[1,5-a]pyridin-2-yl]amino]-N-(tetrahydropyran-4-ylmethyl)pyridine-3-carboxamide;
5-[2-methyl-5-[[(1S,5R,7s)-3-oxa-9-azabicyclo[3.3.1]nonan-7-yl]oxy]-4-pyridyl]-N-[5-(trifluoro methyl)-2-pyridyl]pyrazolo[1,5-a]pyridin-2-amine;
5-(5-(((1R,5S,7s)-3-oxa-9-azabicyclo[3.3.1]nonan-7-yl)oxy)-2-methylpyridin-4-yl)-N-(5-(trifluoromethyl)pyrazin-2-yl)pyrazolo[1,5-a]pyridin-2-amine;
6-[[5-[2-methyl-5-[[(1R,5S,7s)-3-oxa-9-azabicyclo[3.3.1]nonan-7-yl]oxy]-4-pyridyl]pyrazolo[1,5-a]pyridin-2-yl]amino]pyridine-3-carboxamide;
6-[[5-[2-methyl-5-[[(1R,5S,7s)-3-oxa-9-azabicyclo[3.3.1]nonan-7-yl]oxy]-4-pyridyl]pyrazolo[1,5-a]pyridin-2-yl]amino]-N-propyl-pyridine-3-carboxamide;
5-[2-methyl-5-[[(1S,5R,7s)-3-oxa-9-azabicyclo[3.3.1]nonan-7-yl]oxy]-4-pyridyl]-N-[4-(trifluoro methyl)-2-pyridyl]pyrazolo[1,5-a]pyridin-2-amine;
5-[2-methyl-5-[[(1S,5R,7s)-3-oxa-9-azabicyclo[3.3.1]nonan-7-yl]oxy]-4-pyridyl]-N-[1-methyl-5-(trifluoromethyl)pyrazol-3-yl]pyrazolo[1,5-a]pyridin-2-amine;
5-[2-methyl-5-[[(1R,5S,7s)-3-oxa-9-azabicyclo[3.3.1]nonan-7-yl]oxy]-4-pyridyl]-N-(1-methyltriazol-4-yl)pyrazolo[1,5-a]pyridin-2-amine;
5-[2-methyl-5-[[(1S,5R,7s)-3-oxa-9-azabicyclo[3.3.1]nonan-7-yl]oxy]-4-pyridyl]-N-(5-methylpyrazin-2-yl)pyrazolo[1,5-a]pyridin-2-amine;
N-(2,6-dimethylpyrimidin-4-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine;
N,N-dimethyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
N-ethyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
N-ethyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridazine-3-carboxamide;
N-ethyl-5-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyrazine-2-carboxamide;
N-(2-methoxyethyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]-N-tetrahydropyran-4-yl-pyridine-3-carboxamide;
N-(2-cyano-2-methyl-propyl)-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridine-3-carboxamide;
N-(2-hydroxy-2-methyl-propyl)-5-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyrazine-2-carboxamide;
5-(5-(((1R,5S,7s)-3-oxa-9-azabicyclo[3.3.1]nonan-7-yl)oxy)-2-methylpyridin-4-yl)-N-(5-methyl pyrimidin-2-yl)pyrazolo[1,5-a]pyridin-2-amine;
5-[2-methyl-5-[[(1S,5R,7s)-3-oxa-9-azabicyclo[3.3.1]nonan-7-yl]oxy]-4-pyridyl]-N-pyrazin-2-yl-pyrazolo[1,5-a]pyridin-2-amine;
5-[2-methyl-5-[[(1R,5S,7s)-3-oxa-9-azabicyclo[3.3.1]nonan-7-yl]oxy]-4-pyridyl]-N-(5-methylsulfonyl-2-pyridyl)pyrazolo[1,5-a]pyridin-2-amine
5-[2-methyl-5-[[(1R,5S,7s)-3-oxa-9-azabicyclo[3.3.1]nonan-7-yl]oxy]-4-pyridyl]-N-pyridazin-3-yl-pyrazolo[1,5-a]pyridin-2-amine;
5-[2-methyl-5-[[(1R,5S,7s)-3-oxa-9-azabicyclo[3.3.1]nonan-7-yl]oxy]-4-pyridyl]-N-pyrimidin-4-yl-pyrazolo[1,5-a]pyridin-2-amine;
5-[2-methyl-5-[[(1R,5S,7s)-3-oxa-9-azabicyclo[3.3.1]nonan-7-yl]oxy]-4-pyridyl]-N-(6-methylpyrimidin-4-yl)pyrazolo[1,5-a]pyridin-2-amine;
N-(5-fluoro-1-methyl-pyrazol-3-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine;
N-(3-fluoro-1-methyl-pyrazol-4-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine;
N-(4-fluoro-1-methyl-pyrazol-3-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine;
N-[3-methyl-5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]cyclopropanecarboxamide;
2-methyl-6-[[5-[2-methyl-5-[[(1S,5R,7s)-3-oxa-9-azabicyclo[3.3.1]nonan-7-yl]oxy]-4-pyridyl]pyrazolo[1,5-a]pyridin-2-yl]amino]pyridazin-3-one;
5-[2-methyl-5-[[(1S,5R,7s)-3-oxa-9-azabicyclo[3.3.1]nonan-7-yl]oxy]-4-pyridyl]-N-pyrimidin-2-yl-pyrazolo[1,5-a]pyridin-2-amine;
5-(5-(((1R,5S,7s)-3-oxa-9-azabicyclo[3.3.1]nonan-7-yl)oxy)-2-methylpyridin-4-yl)-N-(1-methyl-3-(trifluoromethyl)-1H-pyrazol-5-yl)pyrazolo[1,5-a]pyridin-2-amine;
N-(5-fluoropyrimidin-2-yl)-5-[2-methyl-5-[[(1S,5R,7s)-3-oxa-9-azabicyclo[3.3.1]nonan-7-yl]oxy]-4-pyridyl]pyrazolo[1,5-a]pyridin-2-amine;
5-(5-(((1R,5S,7s)-3-oxa-9-azabicyclo[3.3.1]nonan-7-yl)oxy)-2-methylpyridin-4-yl)pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-[6-(trifluoromethyl)-2-pyridyl]pyrazolo[1,5-a]pyridin-2-amine;
N-methyl-6-[[5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-yl]amino]pyridine-2-carboxamide;
5-[2-methyl-5-[[(1S,5R,7s)-9-methyl-3-oxa-9-azabicyclo[3.3.1]nonan-7-yl]oxy]-4-pyridyl]-N-pyridazin-4-yl-pyrazolo[1,5-a]pyridin-2-amine;
N-methyl-2-[[5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-yl]amino]pyridine-4-carboxamide;
5-[2-methyl-5-[[(1R,5S,7s)-9-methyl-3-oxa-9-azabicyclo[3.3.1]nonan-7-yl]oxy]-4-pyridyl]-N-(2-methylpyrimidin-4-yl)pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-[5-(trifluoromethyl)-2-pyridyl]pyrazolo[1,5-a]pyridin-2-amine;
N-(3-fluoro-1-methyl-pyrazol-4-yl)-5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-amine;
5-[2-methyl-5-[[(1R,5S,7s)-9-methyl-3-oxa-9-azabicyclo[3.3.1]nonan-7-yl]oxy]-4-pyridyl]-N-[4-(trifluoromethyl)-2-pyridyl]pyrazolo[1,5-a]pyridin-2-amine;
5-[2-methyl-5-[[(1R,5S,7s)-9-methyl-3-oxa-9-azabicyclo[3.3.1]nonan-7-yl]oxy]-4-pyridyl]-N-(1-methyltriazol-4-yl)pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-pyrimidin-5-yl-pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-(5-methylpyrazin-2-yl)pyrazolo[1,5-a]pyridin-2-amine;
N-(2,6-dimethylpyrimidin-4-yl)-5-[2-methyl-5-[[(1S,5R,7s)-9-methyl-3-oxa-9-azabicyclo[3.3.1]nonan-7-yl]oxy]-4-pyridyl]pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-pyrazin-2-yl-pyrazolo[1,5-a]pyridin-2-amine;
5-[2-methyl-5-[[(1R,5S,7s)-9-methyl-3-oxa-9-azabicyclo[3.3.1]nonan-7-yl]oxy]-4-pyridyl]-N-pyridazin-3-yl-pyrazolo[1,5-a]pyridin-2-amine;
5-[2-methyl-5-[[(1R,5S,7s)-9-methyl-3-oxa-9-azabicyclo[3.3.1]nonan-7-yl]oxy]-4-pyridyl]-N-pyrimidin-4-yl-pyrazolo[1,5-a]pyridin-2-amine;
5-[2-methyl-5-[[(1R,5S,7s)-9-methyl-3-oxa-9-azabicyclo[3.3.1]nonan-7-yl]oxy]-4-pyridyl]-N-(6-methylpyrimidin-4-yl)pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-(1-methylpyrazol-3-yl)pyrazolo[1,5-a]pyridin-2-amine;
5-[2-methyl-5-[[(1S,5R,7s)-9-methyl-3-oxa-9-azabicyclo[3.3.1]nonan-7-yl]oxy]-4-pyridyl]-N-(1-methylpyrazol-3-yl)pyrazolo[1,5-a]pyridin-2-amine;
5-[2-methyl-5-[[(2S)-morpholin-2-yl]methoxy]-4-pyridyl]-N-[5-(trifluoromethyl)-2-pyridyl]pyrazolo[1,5-a]pyridin-2-amine;
5-[2-methyl-5-[[(2S)-morpholin-2-yl]methoxy]-4-pyridyl]-N-(5-methyl-2-pyridyl)pyrazolo[1,5-a]pyridin-2-amine;
5-[2-methyl-5-[[(2S)-morpholin-2-yl]methoxy]-4-pyridyl]-N-(6-methyl-2-pyridyl)pyrazolo[1,5-a]pyridin-2-amine;
5-[2-methyl-5-[[(2S)-morpholin-2-yl]methoxy]-4-pyridyl]-N-(4-methyl-2-pyridyl)pyrazolo[1,5-a]pyridin-2-amine;
5-[2-methyl-5-[[(2R)-morpholin-2-yl]methoxy]-4-pyridyl]-N-(2-pyridyl)pyrazolo[1,5-a]pyridin-2-amine;
5-[2-methyl-5-[[(2R)-morpholin-2-yl]methoxy]-4-pyridyl]-N-(1-methylpyrazol-3-yl)pyrazolo[1,5-a]pyridin-2-amine;
5-[2-methyl-5-[[(2R)-morpholin-2-yl]methoxy]-4-pyridyl]-N-pyrazin-2-yl-pyrazolo[1,5-a]pyridin-2-amine;
5-[2-methyl-5-[[(2S)-morpholin-2-yl]methoxy]-4-pyridyl]-N-pyrazin-2-yl-pyrazolo[1,5-a]pyridin-2-amine;
N-(2,6-dimethylpyrimidin-4-yl)-5-[2-methyl-5-[[(2S)-morpholin-2-yl]methoxy]-4-pyridyl]pyrazolo[1,5-a]pyridin-2-amine;
N-[5-[5-[(3-aminooxetan-3-yl)methoxy]-2-prop-1-ynyl-4-pyridyl]pyrazolo[1,5-a]pyridin-2-yl]cyclopropanecarboxamide;
N-[5-[5-[(3-fluoroazetidin-3-yl)methoxy]-2-prop-1-ynyl-4-pyridyl]pyrazolo[1,5-a]pyridin-2-yl]cyclopropanecarboxamide;
N-(5-(5-((1r,3r)-3-aminocyclobutoxy)-2-(prop-1-yn-1-yl)pyridin-4-yl)pyrazolo[1,5-a]pyridin-2-yl)cyclopropanecarboxamide;
N-[5-[5-[[(1S,5R)-3-oxa-9-azabicyclo[3.3.1]nonan-7-yl]oxy]-2-prop-1-ynyl-4-pyridyl]pyrazolo[1,5-a]pyridin-2-yl]cyclopropanecarboxamide;
5-[5-[(3-fluoroazetidin-3-yl)methoxy]-2-prop-1-ynyl-4-pyridyl]-N-(1-methylpyrazol-3-yl)pyrazolo[1,5-a]pyridin-2-amine;
N-(1-methylpyrazol-3-yl)-5-[5-[[(1S,5R)-3-oxa-9-azabicyclo[3.3.1]nonan-7-yl]oxy]-2-prop-1-yn yl-4-pyridyl]pyrazolo[1,5-a]pyridin-2-amine;
N-[5-[5-[[(1S,5R)-3-oxa-9-azabicyclo[3.3.1]nonan-7-yl]oxy]-2-prop-1-ynyl-4-pyridyl]pyrazolo[1,5-a]pyridin-2-yl]cyclopropanecarboxamide;
N-(2,6-dimethylpyrimidin-4-yl)-5-[5-[[(1S,4S,7R)-2-oxa-5-azabicyclo[2.2.1]heptan-7-yl]oxy]-2-prop-1-ynyl-4-pyridyl]pyrazolo[1,5-a]pyridin-2-amine;
N-[5-[5-[[(3R)-1-methylpyrrolidin-3-yl]methoxy]-2-prop-1-ynyl-4-pyridyl]pyrazolo[1,5-a]pyridin-2-yl]cyclopropanecarboxamide;
N-[5-[5-(1-methylazetidin-3-yl)oxy-2-prop-1-ynyl-4-pyridyl]pyrazolo[1,5-a]pyridin-2-yl]cyclopropanecarboxamide;
N-[5-[5-[[(2S)-morpholin-2-yl]methoxy]-2-prop-1-ynyl-4-pyridyl]pyrazolo[1,5-a]pyridin-2-yl]cyclopropanecarboxamide;
N-(5-(5-((1r,3r)-3-amino-3-methylcyclobutoxy)-2-methylpyridin-4-yl)pyrazolo[1,5-a]pyridin-2-yl)cyclopropanecarboxamide;
N-[5 [2-methyl-5-[[(3*S,5*R)-5-(trifluoromethyl)-3-piperidyl]oxy]-4-pyridyl]pyrazolo[1,5-a]pyridin-2-yl]cyclopropanecarboxamide;
N-[5-[2-methyl-5-[[(3*R,5*S)-5-(trifluoromethyl)-3-piperidyl]oxy]-4-pyridyl]pyrazolo[1,5-a]pyridin-2-yl]cyclopropanecarboxamide;
(*R)—N-[5-[5-[(3-fluoropyrrolidin-3-yl)methoxy]-2-methyl-4-pyridyl]pyrazolo[1,5-a]pyridin-2-yl]cyclopropanecarboxamide;
(*S)—N-[5-[5-[(3-fluoropyrrolidin-3-yl)methoxy]-2-methyl-4-pyridyl]pyrazolo[1,5-a]pyridin-2-yl]cyclopropanecarboxamide;
*R—N-[5-[5-[(3-fluoro-1-methyl-pyrrolidin-3-yl)methoxy]-2-methyl-4-pyridyl]pyrazolo[1,5-a]pyridin-2-yl]cyclopropanecarboxamide;
(*S)—N-[5-[5-[(1,2-dimethylazetidin-2-yl)methoxy]-2-methyl-4-pyridyl]pyrazolo[1,5-a]pyridin-2-yl]cyclopropanecarboxamide;
N-[5-[5-[[(2R)-1-ethylazetidin-2-yl]methoxy]-2-methyl-4-pyridyl]pyrazolo[1,5-a]pyridin-2-yl]cyclopropanecarboxamide;
N-[5-[2-methyl-5-[[(2R)-1-(2,2,2-trifluoroethyl)azetidin-2-yl]methoxy]-4-pyridyl]pyrazolo[1,5-a]pyridin-2-yl]cyclopropanecarboxamide;
(*S, *S)—N-[5-[5-(2-ethylazetidin-3-yl)oxy-2-methyl-4-pyridyl]pyrazolo[1,5-a]pyridin-2-yl]cyclopropanecarboxamide;
(*R)—N-[5-[2-methyl-5-[(2R)-3,3,3-trifluoro-2-hydroxy-propoxy]-4-pyridyl]pyrazolo[1,5-a]pyridin-2-yl]cyclopropanecarboxamide;
*R, *R—N-[5-[5-(1,2-dimethylazetidin-3-yl)oxy-2-methyl-4-pyridyl]pyrazolo[1,5-a]pyridin-2-yl]cyclopropanecarboxamide;
(*S, *S)—N-[5-[5-(1,2-dimethylazetidin-3-yl)oxy-2-methyl-4-pyridyl]pyrazolo[1,5-a]pyridin-2-yl]cyclopropanecarboxamide;
N-[5-[5-[[1-(2-fluoroethyl)azetidin-3-yl]methoxy]-2-methyl-4-pyridyl]pyrazolo[1,5-a]pyridin-2-yl]cyclopropanecarboxamide;
Trans-*R, *R—N-[5-[5-[(4-methoxypyrrolidin-3-yl)methoxy]-2-methyl-4-pyridyl]pyrazolo[1,5-a]pyridin-2-yl]cyclopropanecarboxamide;
Cis-RS—N-[5-[5-[(4-methoxypyrrolidin-3-yl)methoxy]-2-methyl-4-pyridyl]pyrazolo[1,5-a]pyridin-2-yl]cyclopropanecarboxamide;
N-[5-[2-methyl-5-[[(1S,5R)-9-methyl-3-oxa-9-azabicyclo[3.3.1]nonan-7-yl]oxy]-4-pyridyl]pyrazolo[1,5-a]pyridin-2-yl]cyclopropanecarboxamide;
Trans-*R, *R—N-[5-[5-[(4-methoxy-1-methyl-pyrrolidin-3-yl)methoxy]-2-methyl-4-pyridyl]pyrazolo[1,5-a]pyridin-2-yl]cyclopropanecarboxamide;
N-[5-[2-methyl-5-[[(1S,5R)-9-(trideuteriomethyl)-3-oxa-9-azabicyclo[3.3.1]nonan-7-yl]oxy]-4-pyridyl]pyrazolo[1,5-a]pyridin-2-yl]cyclopropanecarboxamide;
(1R,2R)-2-methyl-N-[5-[2-methyl-5-[[(1S,4S,7R)-2-oxa-5-azabicyclo[2.2.1]heptan-7-yl]oxy]-4-pyridyl]pyrazolo[1,5-a]pyridin-2-yl]cyclopropanecarboxamide;
N-(5-(5-((1s,3s)-3-aminocyclobutoxy)-2-methylpyridin-4-yl)pyrazolo[1,5-a]pyridin-2-yl)cyclopropanecarboxamide;
N-[5-[5-[(2R)-2-amino-2-phenyl-ethoxy]-2-methyl-4-pyridyl]pyrazolo[1,5-a]pyridin-2-yl]cyclopropanecarboxamide;
N-[5-[5-[(1S)-2-amino-1-phenyl-ethoxy]-2-methyl-4-pyridyl]pyrazolo[1,5-a]pyridin-2-yl]cyclopropanecarboxamide;
N-[5-[5-[(2S)-2-amino-2-phenyl-ethoxy]-2-methyl-4-pyridyl]pyrazolo[1,5-a]pyridin-2-yl]cyclopropanecarboxamide;
N-[5-[2-methyl-5-[[(2R)-1-methylazetidin-2-yl]methoxy]-4-pyridyl]pyrazolo[1,5-a]pyridin-2-yl]cyclopropanecarboxamide;
rac-N-[5-[5-[(3,3-difluoroazetidin-2-yl)methoxy]-2-methyl-4-pyridyl]pyrazolo[1,5-a]pyridin-2-yl]cyclopropanecarboxamide;
N-[5-[2-methyl-5-[(3-phenylazetidin-3-yl)methoxy]-4-pyridyl]pyrazolo[1,5-a]pyridin-2-yl]cyclopropanecarboxamide;
N-[5-[5-[[(2S)-3,3-dimethylazetidin-2-yl]methoxy]-2-methyl-4-pyridyl]pyrazolo[1,5-a]pyridin-2-yl]cyclopropanecarboxamide;
Racemic-N-[5-[2-methyl-5-(7-oxa-2-azaspiro[3.5]nonan-3-ylmethoxy)-4-pyridyl]pyrazolo[1,5-a]pyridin-2-yl]cyclopropanecarboxamide;
N-[5-[2-methyl-5-[[(2S)-1,3,3-trimethylazetidin-2-yl]methoxy]-4-pyridyl]pyrazolo[1,5-a]pyridin-2-yl]cyclopropanecarboxamide;
N-[5-[5-methyl-2-[[(2S)-morpholin-2-yl]methoxy]-3-pyridyl]pyrazolo[1,5-a]pyridin-2-yl]cyclopropanecarboxamide;
3-[[6-(difluoromethoxy)-4-[2-([1,2,4]triazolo[1,5-a]pyridin-7-ylamino)pyrazolo[1,5-a]pyridin-5-yl]-3-pyridyl]oxy]-2,2-dimethyl-propanenitrile;
N-[5-[5-cyano-2-[[(3R)-pyrrolidin-3-yl]methoxy]phenyl]pyrazolo[1,5-a]pyridin-2-yl]cyclopropanecarboxamide;
(2*R, 1*S)—N-[5-[5-cyano-2-(1-morpholin-2-ylethoxy)phenyl]pyrazolo[1,5-a]pyridin-2-yl]cyclopropanecarboxamide;
4-[[(2R)-morpholin-2-yl]methoxy]-3-[2-(2-pyridylamino)pyrazolo[1,5-a]pyridin-5-yl]benzonitrile;
N-[5-[2-[[(1R,5S)-3-oxa-9-azabicyclo[3.3.1]nonan-7-yl]oxy]-5-prop-1-ynyl-phenyl]pyrazolo[1,5-a]pyridin-2-yl]cyclopropanecarboxamide;
N-[5-[2-[[(1R,5S)-3-oxa-9-azabicyclo[3.3.1]nonan-7-yl]oxy]-5-propanoyl-phenyl]pyrazolo[1,5-a]pyridin-2-yl]cyclopropanecarboxamide;
N-[5-[5-(difluoromethyl)-2-[[(1R,5S)-3-oxa-9-azabicyclo[3.3.1]nonan-7-yl]oxy]phenyl]pyrazolo[1,5-a]pyridin-2-yl]cyclopropanecarboxamide;
N-[5-[5-(difluoromethyl)-2-[[(1R,5S)-9-methyl-3-oxa-9-azabicyclo[3.3.1]nonan-7-yl]oxy]phenyl]pyrazolo[1,5-a]pyridin-2-yl]cyclopropanecarboxamide;
3-[2-[(2,6-dimethylpyrimidin-4-yl)amino]pyrazolo[1,5-a]pyridin-5-yl]-4-(4-hydroxycyclohexoxy) benzonitrile;
3-[2-[(2,6-dimethylpyrimidin-4-yl)amino]pyrazolo[1,5-a]pyridin-5-yl]-4-(2-hydroxy-2-methyl-propoxy)benzonitrile;
N-[5-[6-chloro-3-(1-methylazetidin-3-yl)oxy-pyridazin-4-yl]pyrazolo[1,5-a]pyridin-2-yl]cyclopropanecarboxamide;
5-[6-methyl-3-[[(1S,5R)-3-oxa-9-azabicyclo[3.3.1]nonan-7-yl]oxy]pyridazin-4-yl]-N-pyrazin-2-yl-pyrazolo[1,5-a]pyridin-2-amine;
[4-[4-[2-[(2,6-dimethylpyrimidin-4-yl)amino]pyrazolo[1,5-a]pyridin-5-yl]-6-methyl-pyridazin-3-yl]oxycyclohexyl]acetate;
4-[4-[2-[(2,6-dimethylpyrimidin-4-yl)amino]pyrazolo[1,5-a]pyridin-5-yl]-6-methyl-pyridazin-3-yl]oxycyclohexanol;
N-[5-[3-[[(3R)-1-methylpyrrolidin-3-yl]methoxy]-4-pyridyl]pyrazolo[1,5-a]pyridin-2-yl]cyclopropanecarboxamide;
N-[5-[2-chloro-5-[[(3R)-1-methylpyrrolidin-3-yl]methoxy]-4-pyridyl]pyrazolo[1,5-a]pyridin-2-yl]cyclopropanecarboxamide;
N-[5-[2-chloro-5-(1-methylazetidin-3-yl)oxy-4-pyridyl]pyrazolo[1,5-a]pyridin-2-yl]cyclopropanecarboxamide;
N-[5-[2-chloro-5-[[(1S,5R)-3-oxa-9-azabicyclo[3.3.1]nonan-7-yl]oxy]-4-pyridyl]pyrazolo[1,5-a]pyridin-2-yl]cyclopropanecarboxamide;
1-[[6-(difluoromethyl)-4-[2-[(2,6-dimethylpyrimidin-4-yl)amino]pyrazolo[1,5-a]pyridin-5-yl]-3-pyridyl]oxy]-2-methyl-propan-2-ol;
3-[[4-[2-[(2,6-dimethylpyrimidin-4-yl)amino]pyrazolo[1,5-a]pyridin-5-yl]-6-(trifluoromethyl)-3-pyridyl]oxy]-2,2-dimethyl-propanenitrile
5-[2-chloro-5-[[(1S,4S,7R)-2-oxa-5-azabicyclo[2.2.1]heptan-7-yl]oxy]-4-pyridyl]-N-(2,6-dimeth ylpyrimidin-4-yl)pyrazolo[1,5-a]pyridin-2-amine;
N-(2,6-dimethylpyrimidin-4-yl)-5-[2-methoxy-5-[[(2S)-morpholin-2-yl]methoxy]-4-pyridyl]pyrazolo[1,5-a]pyridin-2-amine;
5-[2-chloro-5-[[(2S)-morpholin-2-yl]methoxy]-4-pyridyl]-N-(2,6-dimethylpyrimidin-4-yl)pyrazolo[1,5-a]pyridin-2-amine;
(2R,3S)-2-[[4-[2-[(2,6-dimethylpyrimidin-4-yl)amino]pyrazolo[1,5-a]pyridin-5-yl]-6-methoxy-3-pyridyl]oxymethyl]tetrahydrofuran-3-ol;
(2R,3S)-2-[[4-[2-[(2,6-dimethylpyrimidin-4-yl)amino]pyrazolo[1,5-a]pyridin-5-yl]-6-(2,2,2-trifluoroethoxy)-3-pyridyl]oxymethyl]tetrahydrofuran-3-ol;
2-[[4-[2-[(2,6-dimethylpyrimidin-4-yl)amino]pyrazolo[1,5-a]pyridin-5-yl]-5-[[(2R,3S)-3-hydroxytetrahydrofuran-2-yl]methoxy]-2-pyridyl]oxy]acetonitrile;
N-(2,6-dimethylpyrimidin-4-yl)-5-[2-(methylamino)-5-[[(2S)-morpholin-2-yl]methoxy]-4-pyridyl]pyrazolo[1,5-a]pyridin-2-amine;
5-[2-(difluoromethoxy)-5-[[(2S)-morpholin-2-yl]methoxy]-4-pyridyl]-N-(2,6-dimethylpyrimidin-4-yl)pyrazolo[1,5-a]pyridin-2-amine;
N-[5-[2-(difluoromethoxy)-5-[[(2S)-morpholin-2-yl]methoxy]-4-pyridyl]pyrazolo[1,5-a]pyridin-2-yl]cyclopropanecarboxamide;
N-[6-[5-(4-hydroxycyclohexoxy)-2-methyl-4-pyridyl]imidazo[1,2-a]pyridin-2-yl]cyclopropanecarboxamide;
N-[6-[2-methyl-5-[[(1S,5R)-3-oxa-9-azabicyclo[3.3.1]nonan-7-yl]oxy]-4-pyridyl]imidazo[1,2-a]pyrazin-2-yl]cyclopropanecarboxamide;
4-[[4-[2-[(2,6-dimethylpyrimidin-4-yl)amino]imidazo[1,2-a]pyrazin-6-yl]-6-methyl-3-pyridyl]oxy]cyclohexanol;
4-[[4-[2-[(2,6-dimethylpyrimidin-4-yl)amino]imidazo[1,2-a]pyrazin-6-yl]-6-methyl-3-pyridyl]oxy]cyclohexanol;
N-[6-[5-(2-cyano-2-methyl-propoxy)-2-methyl-4-pyridyl]imidazo[1,2-a]pyrazin-2-yl]cyclopropanecarboxamide;
N-[5-[2-methyl-5-[[(3R)-1-methylpyrrolidin-3-yl]methoxy]-4-pyridyl]pyrazolo[1,5-a]pyridin-2-yl]cyclopropanecarboxamide;
N-[5-[5-[(3S)-2,2-dimethylazetidin-3-yl]oxy-2-methyl-4-pyridyl]pyrazolo[1,5-a]pyridin-2-yl]cyclopropanecarboxamide;
N-(1-isopropylpyrazol-4-yl)-5-[2-methyl-5-(1-methylazetidin-3-yl)oxy-4-pyridyl]pyrazolo[1,5-a]pyridin-2-amine;
N-[5-[2-methyl-5-[[(2S)-1-methylazetidin-2-yl]methoxy]-4-pyridyl]pyrazolo[1,5-a]pyridin-2-yl]cyclopropanecarboxamide;
5-[2-methyl-5-[[(2S,5R)-5-methylmorpholin-2-yl]methoxy]-4-pyridyl]-N-pyridazin-3-yl-pyrazolo[1,5-a]pyridin-2-amine;
5-[2-methyl-5-[[(1S,5R)-8-methyl-8-azabicyclo[3.2.1]octan-3-yl]oxy]-4-pyridyl]-N-pyridazin-3-yl-pyrazolo[1,5-a]pyridin-2-amine;
5-[2-methyl-5-[[(2S)-4-methylmorpholin-2-yl]methoxy]-4-pyridyl]-N-pyrazin-2-yl-pyrazolo[1,5-a]pyridin-2-amine;
(*R)-5-[5-[(6,6-dimethylmorpholin-2-yl)methoxy]-2-methyl-4-pyridyl]-N-pyridazin-3-yl-pyrazolo[1,5-a]pyridin-2-amine;
N-[5-[5-(3-amino-3-methyl-cyclobutoxy)-2-methyl-4-pyridyl]pyrazolo[1,5-a]pyridin-2-yl]cyclopropanecarboxamide;
5-[5-[[(2S)-6,6-dimethylmorpholin-2-yl]methoxy]-2-methyl-4-pyridyl]-N-pyridazin-3-yl-pyrazolo[1,5-a]pyridin-2-amine;
(*S)—N-[5-[5-[(3-fluoro-1-methyl-pyrrolidin-3-yl)methoxy]-2-methyl-4-pyridyl]pyrazolo[1,5-a]pyridin-2-yl]cyclopropanecarboxamide;
5-[5-(3-aminocyclobutoxy)-2-methyl-4-pyridyl]-N-pyridazin-3-yl-pyrazolo[1,5-a]pyridin-2-amine;
N-(2,6-dimethylpyrimidin-4-yl)-5-[2-methyl-5-[[(2S)-1-methylazetidin-2-yl]methoxy]-4-pyridyl]pyrazolo[1,5-a]pyridin-2-amine;
(*R)—N-[5-[5-[(3-hydroxy-1-methyl-pyrrolidin-3-yl)methoxy]-2-methyl-4-pyridyl]pyrazolo[1,5-a]pyridin-2-yl]cyclopropanecarboxamide;
5-[2-methyl-5-[[(1S,4S,7R)-2-oxa-5-azabicyclo[2.2.1]heptan-7-yl]oxy]-4-pyridyl]-N-(6-methylpyridazin-3-yl)pyrazolo[1,5-a]pyridin-2-amine;
N-(2,6-dimethylpyrimidin-4-yl)-5-[2-methyl-5-[[(1S,4S,7R)-2-oxa-5-azabicyclo[2.2.1]heptan-7-yl]oxy]-4-pyridyl]pyrazolo[1,5-a]pyridin-2-amine;
N-(6-cyclopropylpyridazin-3-yl)-5-[2-methyl-5-[[(1S,4S,7R)-2-oxa-5-azabicyclo[2.2.1]heptan-7-yl]oxy]-4-pyridyl]pyrazolo[1,5-a]pyridin-2-amine;
N-[5-[5-[[(2S)-azetidin-2-yl]methoxy]-2-methyl-4-pyridyl]pyrazolo[1,5-a]pyridin-2-yl]cyclopropanecarboxamide;
N-(2,6-dimethylpyrimidin-4-yl)-5-[2-methyl-5-[[(2R)-1-methylazetidin-2-yl]methoxy]-4-pyridyl]pyrazolo[1,5-a]pyridin-2-amine;
5-[2-methyl-5-[[(1S,4S,7R)-2-oxa-5-azabicyclo[2.2.1]heptan-7-yl]oxy]-4-pyridyl]-N-(5-methylpyrazin-2-yl)pyrazolo[1,5-a]pyridin-2-amine;
N-[5-[5-[[(2R)-1-(2-fluoroethyl)azetidin-2-yl]methoxy]-2-methyl-4-pyridyl]pyrazolo[1,5-a]pyridin-2-yl]cyclopropanecarboxamide;
6-[[5-[5-[[(1S,5R)-9-ethyl-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide;
6-[[5-[5-[[(1S,5R)-9-isopropyl-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide;
6-[[5-[5-[[(1S,5R)-9-(2-hydroxy-2-methyl-propyl)-3-oxa-9-azabicyclo[3.3.1]nonan-7-yl]oxy]-2-methyl-4-pyridyl]pyrazolo[1,5-a]pyridin-2-yl]amino]-N-methyl-pyridine-3-carboxamide;
(*S)—N-(2,6-dimethylpyrimidin-4-yl)-5-[5-(5,5-dimethyltetrahydrofuran-3-yl)oxy-2-methyl-4-pyridyl]pyrazolo[1,5-a]pyridin-2-amine;
(3*R, 4*R)—N-(2,6-dimethylpyrimidin-4-yl)-5-[2-methyl-5-(4-methyltetrahydrofuran-3-yl)oxy-4-pyrid yl]pyrazolo[1,5-a]pyridin-2-amine;
(*R)—N-(2,6-dimethylpyrimidin-4-yl)-5-[2-methyl-5-[(3-methyltetrahydrofuran-3-yl)methoxy]-4-pyridyl]pyrazolo[1,5-a]pyridin-2-amine;
(*S)—N-(2,6-dimethylpyrimidin-4-yl)-5-[2-methyl-5-[(3-methyltetrahydrofuran-3-yl)methoxy]-4-pyridyl]pyrazolo[1,5-a]pyridin-2-amine;
2-methyl-1-[[6-methyl-4-[2-[[6-methyl-2-(trifluoromethyl)pyrimidin-4-yl]amino]pyrazolo[1,5-a]pyridin-5-yl]-3-pyridyl]oxy]propan-2-ol;
(1r,3r)-3-(((4-(2-((2,6-dimethylpyrimidin-4-yl)amino)pyrazolo[1,5-a]pyridin-5-yl)-6-methylpyridin-3-yl)oxy)methyl)cyclobutan-1-ol;
(*R)—N-(2,6-dimethylpyrimidin-4-yl)-5-[5-[(5,5-dimethyltetrahydrofuran-3-yl)methoxy]-2-methyl-4-pyridyl]pyrazolo[1,5-a]pyridin-2-amine;
(*S)—N-(2,6-dimethylpyrimidin-4-yl)-5-[5-[(5,5-dimethyltetrahydrofuran-3-yl)methoxy]-2-methyl-4-pyridyl]pyrazolo[1,5-a]pyridin-2-amine;
2-methyl-1-[[6-methyl-4-[2-[(5-methylpyrazin-2-yl)amino]pyrazolo[1,5-a]pyridin-5-yl]-3-pyridyl]oxy]propan-2-ol;
(rac-)-4-[[4-[2-[(2,6-dimethylpyrimidin-4-yl)amino]pyrazolo[1,5-a]pyridin-5-yl]-6-methyl-3-pyridyl]oxymethyl]-1-methyl-pyrrolidin-2-one;
(*R)—N-(2,6-dimethylpyrimidin-4-yl)-5-[5-(6,9-dioxaspiro[4.4]nonan-8-ylmethoxy)-2-methyl-4-pyridyl]pyrazolo[1,5-a]pyridin-2-amine;
(*S)—N-(2,6-dimethylpyrimidin-4-yl)-5-[5-(6,9-dioxaspiro[4.4]nonan-8-ylmethoxy)-2-methyl-4-pyridyl]pyrazolo[1,5-a]pyridin-2-amine;
5-(5-((1r,3r)-3-aminocyclobutoxy)-2-methylpyridin-4-yl)-N-(2,6-dimethylpyrimidin-4-yl)pyrazolo[1,5-a]pyridin-2-amine;
N-(2,6-dimethylpyrimidin-4-yl)-5-[2-methyl-5-[(3-methyl oxetan-3-yl)methoxy]-4-pyridyl]pyrazolo[1,5-a]pyridin-2-amine;
(*R)-4-[[4-[2-[(2,6-dimethylpyrimidin-4-yl)amino]pyrazolo[1,5-a]pyridin-5-yl]-6-methyl-3-pyridyl]oxymethyl]-1-methyl-pyrrolidin-2-one;
(*R)-2-[[4-[2-[(2,6-dimethylpyrimidin-4-yl)amino]pyrazolo[1,5-a]pyridin-5-yl]-6-methyl-3-pyridyl]oxy]-1-(1-methyl cyclopropyl)ethanol;
(*S)-2-[[4-[2-[(2,6-dimethylpyrimidin-4-yl)amino]pyrazolo[1,5-a]pyridin-5-yl]-6-methyl-3-pyridyl]oxy]-1-(1-methylcyclopropyl)ethanol;
N-(2,6-dimethylpyrimidin-4-yl)-5-[5-(1,1-dioxothian-4-yl)oxy-2-methyl-4-pyridyl]pyrazolo[1,5-a]pyridin-2-amine;
N-(2,6-dimethylpyrimidin-4-yl)-5-[2-methyl-5-(4-methyl sulfonyl cyclohexoxy)-4-pyridyl]pyrazolo[1,5-a]pyridin-2-amine;
4-[[4-[2-[(2,6-dimethylpyrimidin-4-yl)amino]pyrazolo[1,5-a]pyridin-5-yl]-6-methyl-3-pyridyl]oxy]cyclohexanecarbonitrile;
5-[5-[[2-(difluoromethoxy)phenyl]methoxy]-2-methyl-4-pyridyl]-N-(2,6-dimethylpyrimidin-4-yl) pyrazolo[1,5-a]pyridin-2-amine;
(3S)-4-[[4-[2-[(2,6-dimethylpyrimidin-4-yl)amino]pyrazolo[1,5-a]pyridin-5-yl]-6-methyl-3-pyrid yl]oxy]-2,3-dimethyl-butan-2-ol;
4-[[4-[2-[(2,6-dimethylpyrimidin-4-yl)amino]pyrazolo[1,5-a]pyridin-5-yl]-6-methyl-3-pyridyl]oxy]-2-methyl-butan-2-ol;
(1s,4s)-4-((4-(2-((2,6-dimethylpyrimidin-4-yl)amino)pyrazolo[1,5-a]pyridin-5-yl)-6-methylpyridin-3-yl)oxy)cyclohexane-1-carbonitrile;
5-[5-(2-aminoethoxy)-2-methyl-4-pyridyl]-N-(2,6-dimethylpyrimidin-4-yl)pyrazolo[1,5-a]pyridin-2-amine;
(1R)-1-cyclopropyl-2-[[4-[2-[(2,6-dimethylpyrimidin-4-yl)amino]pyrazolo[1,5-a]pyridin-5-yl]-6-methyl-3-pyridyl]oxy]ethanol;
(1s)-1-cyclopropyl-2-[[4-[2-[(2,6-dimethylpyrimidin-4-yl)amino]pyrazolo[1,5-a]pyridin-5-yl]-6-methyl-3-pyridyl]oxy]ethanol;
1-[[4-[2-[(2,6-dimethylpyrimidin-4-yl)amino]pyrazolo[1,5-a]pyridin-5-yl]-6-methyl-3-pyridyl]oxymethyl]cyclopropanecarbonitrile;
2-cyclopropyl-6-[[5-[5-[[(2S)-1,4-dioxan-2-yl]methoxy]-2-methyl-4-pyridyl]pyrazolo[1,5-a]pyridin-2-yl]amino]-4-methyl-pyridazin-3-one;
(4S)-4-[[4-[2-[(2,6-dimethylpyrimidin-4-yl)amino]pyrazolo[1,5-a]pyridin-5-yl]-6-methyl-3-pyridyl]oxymethyl]-1,3-dimethyl-imidazolidin-2-one;
N-[5-[2-cyano-5-[[(3R)-1-(cyanomethyl)pyrrolidin-3-yl]methoxy]-4-pyridyl]pyrazolo[1,5-a]pyridin-2-yl]cyclopropanecarboxamide;
5-[2-[(1-methylpyrazol-3-yl)amino]pyrazolo[1,5-a]pyridin-5-yl]-6-[[(2S)-morpholin-2-yl]methoxy]pyridine-3-carbonitrile;
N-[5-[2-cyano-5-[[(1R,5S)-9-methyl-3-oxa-9-azabicyclo[3.3.1]nonan-7-yl]amino]-4-pyridyl]pyrazolo[1,5-a]pyridin-2-yl]cyclopropanecarboxamide;
3-[2-[(1-methylpyrazol-3-yl)amino]pyrazolo[1,5-a]pyridin-5-yl]-4-[[(2R)-morpholin-2-yl]methoxy]benzonitrile;
5-[2-methyl-5-(1-methylazetidin-3-yl)oxy-4-pyridyl]-N-(2-pyridyl)pyrazolo[1,5-a]pyridin-2-amine;
5-[2-methyl-5-(1-methylazetidin-3-yl)oxy-4-pyridyl]-N-(1-methylpyrazol-4-yl)pyrazolo[1,5-a]pyridin-2-amine;
(1*R, 3*R)—N-[5-[5-(3-hydroxycyclopentoxy)-2-methyl-4-pyridyl]pyrazolo[1,5-a]pyridin-2-yl]cyclopropanecarboxamide;
N-[5-[5-[(2R)-2-amino-3,3,3-trifluoro-propoxy]-2-methyl-4-pyridyl]pyrazolo[1,5-a]pyridin-2-yl]cyclopropanecarboxamide;
(3RS, 4RS)—N-[5-[5-[(4-methoxy-1-methyl-pyrrolidin-3-yl)methoxy]-2-methyl-4-pyridyl]pyrazolo[1,5-a]pyridin-2-yl]cyclopropanecarboxamide;
N-[5-[2-methyl-5-[[(2S)-1-methylpyrrolidin-2-yl]methoxy]-4-pyridyl]pyrazolo[1,5-a]pyridin-2-yl]cyclopropanecarboxamide;
N-[5-[5-[[(2S)-1-(2-fluoroethyl)azetidin-2-yl]methoxy]-2-methyl-4-pyridyl]pyrazolo[1,5-a]pyridin-2-yl]cyclopropanecarboxamide;
N-[5-[5-[[(2R)-1-cyclopropylazetidin-2-yl]methoxy]-2-methyl-4-pyridyl]pyrazolo[1,5-a]pyridin-2-yl]cyclopropanecarboxamide;
N-[5-[2-methyl-5-[[(2S)-1-(trideuteriomethyl)azetidin-2-yl]methoxy]-4-pyridyl]pyrazolo[1,5-a]pyridin-2-yl]cyclopropanecarboxamide;
N-[5-[2-methyl-5-[[(2R)-1-(trideuteriomethyl)azetidin-2-yl]methoxy]-4-pyridyl]pyrazolo[1,5-a]pyridin-2-yl]cyclopropanecarboxamide;
(*R)—N-(2,6-dimethylpyrimidin-4-yl)-5-[5-(5,5-dimethyltetrahydrofuran-3-yl)oxy-2-methyl-4-pyridyl]pyrazolo[1,5-a]pyridin-2-amine;
(*S)—N-(2,6-dimethylpyrimidin-4-yl)-5-[5-(2,2-dimethyltetrahydropyran-4-yl)oxy-2-methyl-4-pyridyl]pyrazolo[1,5-a]pyridin-2-amine;
N-[3-[[4-[2-[(2,6-dimethylpyrimidin-4-yl)amino]pyrazolo[1,5-a]pyridin-5-yl]-6-methyl-3-pyridyl]oxy]cyclobutyl]methanesulfonamide;
N-[3-[[4-[2-[(2,6-dimethylpyrimidin-4-yl)amino]pyrazolo[1,5-a]pyridin-5-yl]-6-methyl-3-pyridyl]oxy]cyclobutyl]methanesulfonamide;
(1r,4r)-4-((4-(2-((2,6-dimethylpyrimidin-4-yl)amino)pyrazolo[1,5-a]pyridin-5-yl)-6-methylpyridin-3-yl)oxy)cyclohexane-1-carbonitrile;
(3*R, 6*R)-6-[[4-[2-[(2,6-dimethylpyrimidin-4-yl)amino]pyrazolo[1,5-a]pyridin-5-yl]-6-methyl-3-pyridyl]oxymethyl]tetrahydropyran-3-ol;
N-[5-(2-cyano-5-ethoxy-4-pyridyl)pyrazolo[1,5-a]pyridin-2-yl]cyclopropanecarboxamide;
N-[5-[2-cyano-5-(cyclopropylmethoxy)-4-pyridyl]pyrazolo[1,5-a]pyridin-2-yl]cyclopropanecarboxamide;
N-[5-[2-cyano-5-[[(3R)-3-piperidyl]methoxy]-4-pyridyl]pyrazolo[1,5-a]pyridin-2-yl]cyclopropanecarboxamide;
N-(5-(5-(((1r,4r)-4-aminocyclohexyl)methoxy)-2-cyanopyridin-4-yl)pyrazolo[1,5-a]pyridin-2-yl) cyclopropanecarboxamide;
N-[5-[5-(6-azaspiro[3.3]heptan-2-yloxy)-2-cyano-4-pyridyl]pyrazolo[1,5-a]pyridin-2-yl]cyclopropanecarboxamide;
N-[5-[2-cyano-5-[[(3R)-1-(2,2,2-trifluoroethyl)pyrrolidin-3-yl]methoxy]-4-pyridyl]pyrazolo[1,5-a]pyridin-2-yl]cyclopropanecarboxamide;
N-[5-[2-cyano-5-[[(3R)-1-cyclopropylpyrrolidin-3-yl]methoxy]-4-pyridyl]pyrazolo[1,5-a]pyridin-2-yl]cyclopropanecarboxamide;
N-[5-[2-cyano-5-[1-(2,2,2-trifluoroethyl)azetidin-3-yl]oxy-4-pyridyl]pyrazolo[1,5-a]pyridin-2-yl]cyclopropanecarboxamide;
N-(5-(5-(((1r,3r)-3-aminocyclobutyl)methoxy)-2-cyanopyridin-4-yl)pyrazolo[1,5-a]pyridin-2-yl) cyclopropanecarboxamide;
N-[5-[2-cyano-5-[[(2R)-morpholin-2-yl]methoxy]-4-pyridyl]pyrazolo[1,5-a]pyridin-2-yl]cyclopropanecarboxamide;
N-[5-[2-cyano-5-[[(3S)-3-piperidyl]methoxy]-4-pyridyl]pyrazolo[1,5-a]pyridin-2-yl]cyclopropanecarboxamide;
N-[5-[2-cyano-5-[(3R)-pyrrolidin-3-yl]oxy-4-pyridyl]pyrazolo[1,5-a]pyridin-2-yl]cyclopropanes arboxamide;
(1*S, 4*S, 5*R)—N-[5-[2-cyano-5-[(2-methyl-2-azabicyclo[2.2.1]heptan-5-yl)oxy]-4-pyridyl]pyrazolo[1,5-a]pyridin-2-yl]cyclopropanecarboxamide;
N-[5-[2-cyano-5-[[3-(cyanomethylamino)cyclobutyl]methoxy]-4-pyridyl]pyrazolo[1,5-a]pyridin-2-yl]cyclopropanecarboxamide;
N-[5-[2-cyano-5-[[3-(cyclopropylamino)cyclobutyl]methoxy]-4-pyridyl]pyrazolo[1,5-a]pyridin-2-yl]cyclopropanecarboxamide;
ethyl 2-[[3-[[6-cyano-4-[2-(cyclopropanecarbonylamino)pyrazolo[1,5-a]pyridin-5-yl]-3-pyridyl]oxymethyl]cyclobutyl]amino]acetate;
N-[5-[5-[[3-(b enzylamino)cyclobutyl]methoxy]-2-cyano-4-pyridyl]pyrazolo[1,5-a]pyridin-2-yl]cyclopropanecarboxamide;
1-[4-[2-[(2,6-dimethylpyrimidin-4-yl)amino]pyrazolo[1,5-a]pyridin-5-yl]-6-methyl-pyridazin-3-yl]oxy-2-methyl-propan-2-ol;
N-[5-[5-[[(2S)-morpholin-2-yl]methoxy]-2-(trifluoromethyl)-4-pyridyl]pyrazolo[1,5-a]pyridin-2-yl]cyclopropanecarboxamide;
(1*R, 3*S)—N-[5-[5-(3-hydroxycyclohexoxy)-2-methyl-4-pyridyl]pyrazolo[1,5-a]pyridin-2-yl]cyclopropanecarboxamide;
(*S)—N-[5-[5-(2-cyclopropyl-2-hydroxy-propoxy)-2-methyl-4-pyridyl]pyrazolo[1,5-a]pyridin-2-yl]cyclopropanecarboxamide;
(*R)—N-[5-[5-[(1,2-dimethylazetidin-2-yl)methoxy]-2-methyl-4-pyridyl]pyrazolo[1,5-a]pyridin-2-yl]cyclopropanecarboxamide;
N-[5-[5-[[(2S)-1-ethylazetidin-2-yl]methoxy]-2-methyl-4-pyridyl]pyrazolo[1,5-a]pyridin-2-yl]cyclopropanecarboxamide;
(3S,5S)-5-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol;
(*R)—N-(2,6-dimethylpyrimidin-4-yl)-5-[2-methyl-5-(7-oxaspiro[3.4]octan-6-ylmethoxy)-4-pyrid yl]pyrazolo[1,5-a]pyridin-2-amine;
(*S)-3-[[4-[2-[(2,6-dimethylpyrimidin-4-yl)amino]pyrazolo[1,5-a]pyridin-5-yl]-6-methyl-3-pyrid yl]oxymethyl]tetrahydropyran-3-ol;
(3S,5R)-5-[[4-[2-[(2,6-dimethylpyrimidin-4-yl)amino]pyrazolo[1,5-a]pyridin-5-yl]-6-methyl-3-pyridyl]oxymethyl]tetrahydrofuran-3-ol;
(*S)—N-(2,6-dimethylpyrimidin-4-yl)-5-[5-(1,1-dioxothiazinan-4-yl)oxy-2-methyl-4-pyridyl]pyrazolo[1,5-a]pyridin-2-amine;
5-[2-methyl-5-[[(2R)-4-methylmorpholin-2-yl]methoxy]-4-pyridyl]-N-pyrazin-2-yl-pyrazolo[1,5-a]pyridin-2-amine;
2-methyl-1-[[6-methyl-4-[2-[(6-methylpyridazin-3-yl)amino]pyrazolo[1,5-a]pyridin-5-yl]-3-pyridyl]oxy]propan-2-ol;
1-[[4-[2-[(5-tert-butylpyrazin-2-yl)amino]pyrazolo[1,5-a]pyridin-5-yl]-6-methyl-3-pyridyl]oxy]-2-methyl-propan-2-ol;
N-cyclopropyl-2,6-difluoro-4-[[5-[5-(4-hydroxycyclohexoxy)-2-methyl-4-pyridyl]pyrazolo[1,5-a]pyridin-2-yl]amino]benzamide;
N-cyclopropyl-2-fluoro-4-[[5-[5-(2-hydroxy-2-methyl-propoxy)-2-methyl-4-pyridyl]pyrazolo[1,5-a]pyridin-2-yl]amino]benzamide;
N-[5-[2-cyano-5-[[(3R)-1-ethylpyrrolidin-3-yl]methoxy]-4-pyridyl]pyrazolo[1,5-a]pyridin-2-yl]cyclopropanecarboxamide;
N-(6-(5-(((1R,5S,7s)-3-oxa-9-azabicyclo[3.3.1]nonan-7-yl)oxy)-2-methylpyridin-4-yl)imidazo[1,2-a]pyridin-2-yl)cyclopropanecarboxamide;
N-(5-(5-(((1R,5S,7s)-3-oxa-9-azabicyclo[3.3.1]nonan-7-yl)oxy)-2-(trifluoromethyl)pyridin-4-yl) pyrazolo[1,5-a]pyridin-2-yl)cyclopropanecarboxamide;
N-(5-(5-(((1R,5S,7s)-9-methyl-3-oxa-9-azabicyclo[3.3.1]nonan-7-yl)oxy)-2-(trifluoromethyl)pyridin-4-yl)pyrazolo[1,5-a]pyridin-2-yl)cyclopropanecarboxamide;
N-[5-[5-[(1-amino-3,3-difluoro-cyclobutyl)methoxy]-2-(trifluoromethyl)-4-pyridyl]pyrazolo[1,5-a]pyridin-2-yl]cyclopropanecarboxamide;
N-[5-[5-[(3-fluoroazetidin-3-yl)methoxy]-2-(trifluoromethyl)-4-pyridyl]pyrazolo[1,5-a]pyridin-2-yl]cyclopropanecarboxamide;
N-[5-[5-[(3-methoxyazetidin-3-yl)methoxy]-2-(trifluoromethyl)-4-pyridyl]pyrazolo[1,5-a]pyridin-2-yl]cyclopropanecarboxamide;
1-fluoro-N-[5-[2-methyl-5-[[(2R)-1-methylazetidin-2-yl]methoxy]-4-pyridyl]pyrazolo[1,5-a]pyridin-2-yl]cyclopropanecarboxamide;
2,2-difluoro-N-[5-[2-methyl-5-[[(2R)-1-methylazetidin-2-yl]methoxy]-4-pyridyl]pyrazolo[1,5-a]pyridin-2-yl]cyclopropanecarboxamide;
N-(2,6-dimethylpyrimidin-4-yl)-5-[5-[[(2R)-1-methylazetidin-2-yl]methoxy]-2-(trifluoromethyl)-4-pyridyl]pyrazolo[1,5-a]pyridin-2-amine;
N-cyclopropyl-4-[[5-[5-(2-hydroxy-2-methyl-propoxy)-2-methyl-4-pyridyl]pyrazolo[1,5-a]pyridin-2-yl]amino]-2,6-dimethoxy-benzamide;
1-((1R,5S,7s)-7-((4-(2-((2,6-dimethylpyrimidin-4-yl)amino)pyrazolo[1,5-a]pyridin-5-yl)-6-meth ylpyridin-3-yl)oxy)-3-oxa-9-azabicyclo[3.3.1]nonan-9-yl)ethan-1-one;
1-[(2S)-2-[[4-[2-[(2,6-dimethylpyrimidin-4-yl)amino]pyrazolo[1,5-a]pyridin-5-yl]-6-methyl-3-pyridyl]oxymethyl]morpholin-4-yl]ethanone;
N-(2,6-dimethylpyrimidin-4-yl)-5-[2-methyl-5-[[(2S)-4-methylsulfonylmorpholin-2-yl]methoxy]-4-pyridyl]pyrazolo[1,5-a]pyridin-2-amine;
(S)-5-(2-methyl-5-((4-(methylsulfonyl)morpholin-2-yl)methoxy)pyridin-4-yl)-N-(2-methylpyrimidin-4-yl)pyrazolo[1,5-a]pyridin-2-amine;
2-((5-(5-(((1R,5S,7s)-9-acetyl-3-oxa-9-azabicyclo[3.3.1]nonan-7-yl)oxy)-2-methylpyridin-4-yl)pyrazolo[1,5-a]pyridin-2-yl)amino)-N-methylisonicotinamide;
1-((1R,5S,7s)-7-((6-methyl-4-(2-((4-(trifluoromethyl)pyridin-2-yl)amino)pyrazolo[1,5-a]pyridin-5-yl)pyridin-3-yl)oxy)-3-oxa-9-azabicyclo[3.3.1]nonan-9-yl)ethan-1-one;
2-[[5-[5-(2-hydroxy-2-methyl-propoxy)-2-methyl-4-pyridyl]pyrazolo[1,5-a]pyridin-2-yl]amino]-N,N,6-trimethyl-pyridine-4-carboxamide;
2-((5-(5-(((1R,5S,7s)-9-(cyclopropanecarbonyl)-3-oxa-9-azabicyclo[3.3.1]nonan-7-yl)oxy)-2-methylpyridin-4-yl)pyrazolo[1,5-a]pyridin-2-yl)amino)-N-methylisonicotinamide;
2-((5-(5-(((1R,5S,7s)-9-(cyclopropylsulfonyl)-3-oxa-9-azabicyclo[3.3.1]nonan-7-yl)oxy)-2-meth ylpyridin-4-yl)pyrazolo[1,5-a]pyridin-2-yl)amino)-N-methylisonicotinamide;
N-cyclopropyl-2-[[5-[5-(2-hydroxy-2-methyl-propoxy)-2-methyl-4-pyridyl]pyrazolo[1,5-a]pyridin-2-yl]amino]-6-methyl-pyridine-4-carboxamide;
6-((5-(5-(((1R,5S,7s)-9-acetyl-3-oxa-9-azabicyclo[3.3.1]nonan-7-yl)oxy)-2-methylpyridin-4-yl)pyrazolo[1,5-a]pyridin-2-yl)amino)-2-methylpyridazin-3(2H)-one;
N-cyclopropyl-6-[[5-[5-(2-hydroxy-2-methyl-propoxy)-2-methyl-4-pyridyl]pyrazolo[1,5-a]pyridin-2-yl]amino]-4-methyl-pyridine-2-carboxamide;
N-cyclopropyl-6-[[5-[5-(2-hydroxy-2-methyl-propoxy)-2-methyl-4-pyridyl]pyrazolo[1,5-a]pyridin-2-yl]amino]pyridine-3-carboxamide;
N-cyclopropyl-4-[[5-[5-(2-hydroxy-2-methyl-propoxy)-2-methyl-4-pyridyl]pyrazolo[1,5-a]pyridin-2-yl]amino]-6-methyl-pyrimidine-2-carboxamide;
3-[[6-(difluoromethoxy)-4-[2-[(2-methylpyrimidin-5-yl)amino]pyrazolo[1,5-a]pyridin-5-yl]-3-pyridyl]oxy]-2,2-dimethyl-propanenitrile;
methyl 4-[[5-[5-(2-hydroxy-2-methyl-propoxy)-2-methyl-4-pyridyl]pyrazolo[1,5-a]pyridin-2-yl]amino]-2-methoxy-benzoate;
N-cyclopropyl-4-[[5-[5-(2-hydroxy-2-methyl-propoxy)-2-methyl-4-pyridyl]pyrazolo[1,5-a]pyridin-2-yl]amino]-2-methoxy-benzamide;
4-[[5-[5-(2-hydroxy-2-methyl-propoxy)-2-methyl-4-pyridyl]pyrazolo[1,5-a]pyridin-2-yl]amino]-6-methyl-N-(2,2,2-trifluoroethyl)pyrimidine-2-carboxamide;
(1r,4r)-4-((4-(2-*(3,5-dimethoxyphenyl)amino)pyrazolo[1,5-a]pyridin-5-yl)-6-methylpyridin-3-yl) oxy)cyclohexan-1-ol;
1-[[4-[2-(3,5-dimethoxyanilino)pyrazolo[1,5-a]pyridin-5-yl]-6-methyl-3-pyridyl]oxy]-2-methyl-propan-2-ol;
4-[[5-[5-(2-hydroxy-2-methyl-propoxy)-2-methyl-4-pyridyl]pyrazolo[1,5-a]pyridin-2-yl]amino]-N-(2-hydroxy-2-methyl-propyl)-6-methyl-pyrimidine-2-carboxamide;
N-cyclopropyl-2-(difluoromethoxy)-4-[[5-[5-(2-hydroxy-2-methyl-propoxy)-2-methyl-4-pyridyl]pyrazolo[1,5-a]pyridin-2-yl]amino]-6-methoxy-benzamide;
N-cyclopropyl-2-fluoro-4-((5-(5-(((1r,4r)-4-hydroxycyclohexyl)oxy)-2-methylpyridin-4-yl)pyrazolo[1,5-a]pyridin-2-yl)amino)-6-methoxybenzamide;
6-[[5-[5-(2-hydroxy-2-methyl-propoxy)-2-methyl-4-pyridyl]pyrazolo[1,5-a]pyridin-2-yl]amino]-N-isopropyl-4-(trifluoromethyl)pyridine-3-carboxamide;
6-[[5-[5-(2-hydroxy-2-methyl-propoxy)-2-methyl-4-pyridyl]pyrazolo[1,5-a]pyridin-2-yl]amino]-N-(2,2,2-trifluoroethyl)-4-(trifluoromethyl)pyridine-3-carboxamide;
1-[[4-[2-[(3-fluoro-4-methyl-2-pyridyl)amino]pyrazolo[1,5-a]pyridin-5-yl]-6-methyl-3-pyridyl]oxy]-2-methyl-propan-2-ol;
N-[6-[5-(4-hydroxycyclohexoxy)-2-methyl-4-pyridyl]imidazo[1,2-a]pyrazin-2-yl]cyclopropanecarboxamide;
N-[6-[2-methyl-5-(1-methylazetidin-3-yl)oxy-4-pyridyl]imidazo[1,2-a]pyrazin-2-yl]cyclopropanecarboxamide;
N-[6-[5-(4-hydroxycyclohexoxy)-2-methyl-4-pyridyl]imidazo[1,2-a]pyrazin-2-yl]cyclopropanecarboxamide;
N-[5-[2-cyano-5-[[(1R,5S,7s)-9-methyl-3-oxa-9-azabicyclo[3.3.1]nonan-7-yl]oxy]-4-pyridyl]pyrazolo[1,5-a]pyridin-2-yl]cyclopropanecarboxamide;
4-[[6-methyl-4-[2-[(6-methylpyrazin-2-yl)amino]imidazo[1,2-a]pyrazin-6-yl]-3-pyridyl]oxy]cyclohexanol;
N-(2,6-dimethylpyrimidin-4-yl)-5-[2-ethoxy-5-[[(2S)-morpholin-2-yl]methoxy]-4-pyridyl]pyrazolo[1,5-a]pyridin-2-amine;
3-[[4-[2-[(2,6-dimethylpyrimidin-4-yl)amino]pyrazolo[1,5-a]pyridin-5-yl]-6-(2,2,2-trifluoroethoxy)-3-pyridyl]oxy]-2,2-dimethyl-propanenitrile;
3-[[6-(2,2-difluoroethoxy)-4-[2-[(2,6-dimethylpyrimidin-4-yl)amino]pyrazolo[1,5-a]pyridin-5-yl]-3-pyridyl]oxy]-2,2-dimethyl-propanenitrile;
1-[[4-[2-[(2,6-dimethylpyrimidin-4-yl)amino]pyrazolo[1,5-a]pyridin-5-yl]-6-(2,2,2-trifluoroethoxy)-3-pyridyl]oxy]-2-methyl-propan-2-ol;
N-(2,6-dimethylpyrimidin-4-yl)-5-[5-[[(2S)-morpholin-2-yl]methoxy]-2-(2,2,2-trifluoroethoxy)-4-pyridyl]pyrazolo[1,5-a]pyridin-2-amine;
3-[[4-[2-[(2,6-dimethylpyrimidin-4-yl)amino]pyrazolo[1,5-a]pyridin-5-yl]-6-isopropoxy-3-pyridyl]oxy]-2,2-dimethyl-propanenitrile;
4-[[4-[2-[(2,6-dimethylpyrimidin-4-yl)amino]pyrazolo[1,5-a]pyridin-5-yl]-6-(2,2,2-trifluoroethoxy)-3-pyridyl]oxy]cyclohexanol;
N-[5-[5-(2-cyano-2-methyl-propoxy)-2-(difluoromethoxy)-4-pyridyl]pyrazolo[1,5-a]pyridin-2-yl]cyclopropanecarboxamide;
N-[5-[2-chloro-5-(2-cyano-2-methyl-propoxy)-4-pyridyl]pyrazolo[1,5-a]pyridin-2-yl]cyclopropanecarboxamide;
3-[[6-(2,2-difluoroethylamino)-4-[2-[(2,6-dimethylpyrimidin-4-yl)amino]pyrazolo[1,5-a]pyridin-5-yl]-3-pyridyl]oxy]-2,2-dimethyl-propanenitrile;
3-[[4-[2-[(2,6-dimethylpyrimidin-4-yl)amino]pyrazolo[1,5-a]pyridin-5-yl]-6-(methoxymethyl)-3-pyridyl]oxy]-2,2-dimethyl-propanenitrile;
N-[5-[5-(2-cyano-2-methyl-propoxy)-2-(2,2,2-trifluoroethoxy)-4-pyridyl]pyrazolo[1,5-a]pyridin-2-yl]cyclopropanecarboxamide;
3-[[4-[2-[(2,6-dimethylpyrimidin-4-yl)amino]pyrazolo[1,5-a]pyridin-5-yl]-6-prop-1-ynyl-3-pyridyl]oxy]-2,2-dimethyl-propanenitrile;
(2S,3R)-2-[[4-[2-[(2,6-dimethylpyrimidin-4-yl)amino]pyrazolo[1,5-a]pyridin-5-yl]-6-prop-1-ynyl-3-pyridyl]oxymethyl]tetrahydrofuran-3-ol;
3-[[6-(cyanomethoxy)-4-[2-[(2,6-dimethylpyrimidin-4-yl)amino]pyrazolo[1,5-a]pyridin-5-yl]-3-pyridyl]oxy]-2,2-dimethyl-propanenitrile;
3-[[4-[2-[(2,6-dimethylpyrimidin-4-yl)amino]pyrazolo[1,5-a]pyridin-5-yl]-6-(2-fluoroethoxy)-3-pyridyl]oxy]-2,2-dimethyl-propanenitrile;
3-[[6-hydroxy-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile;
3-[[6-chloro-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile;
3-[[6-(difluoromethoxy)-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile;
(1r,4r)-4-[[6-(difluoromethoxy)-4-[2-[(2,6-dimethylpyrimidin-4-yl)amino]pyrazolo[1,5-a]pyridin-5-yl]-3-pyridyl]oxy]cyclohexanol;
3-[[4-[2-[(2,6-dimethylpyrimidin-4-yl)amino]pyrazolo[1,5-a]pyridin-5-yl]-6-(2-hydroxyethoxy)-3-pyridyl]oxy]-2,2-dimethyl-propanenitrile;
1-[[6-(difluoromethoxy)-4-[2-[(2,6-dimethylpyrimidin-4-yl)amino]pyrazolo[1,5-a]pyridin-5-yl]-3-pyridyl]oxy]-2-methyl-propan-2-ol;
(*R)-3-[[6-(1-cyanoethoxy)-4-[2-[(2,6-dimethylpyrimidin-4-yl)amino]pyrazolo[1,5-a]pyridin-5-yl]-3-pyridyl]oxy]-2,2-dimethyl-propanenitrile;
(*S)-3-[[6-(1-cyanoethoxy)-4-[2-[(2,6-dimethylpyrimidin-4-yl)amino]pyrazolo[1,5-a]pyridin-5-y 1]-3-pyridyl]oxy]-2,2-dimethyl-propanenitrile;
3-[[4-[2-[(2-amino-6-methyl-pyrimidin-4-yl)amino]pyrazolo[1,5-a]pyridin-5-yl]-6-(difluoromethoxy)-3-pyridyl]oxy]-2,2-dimethyl-propanenitrile;
3-[[6-(difluoromethoxy)-4-[2-[(6-methylpyrazin-2-yl)amino]pyrazolo[1,5-a]pyridin-5-yl]-3-pyridyl]oxy]-2,2-dimethyl-propanenitrile;
3-[[6-(difluoromethoxy)-4-[2-[(1,5-dimethyl-6-oxo-pyridazin-3-yl)amino]pyrazolo[1,5-a]pyridin-5-yl]-3-pyridyl]oxy]-2,2-dimethyl-propanenitrile;
3-[[6-(difluoromethoxy)-4-[2-[(6-methylpyrimidin-4-yl)amino]pyrazolo[1,5-a]pyridin-5-yl]-3-pyridyl]oxy]-2,2-dimethyl-propanenitrile;
3-[[4-[2-[(2,6-dimethylpyrimidin-4-yl)amino]pyrazolo[1,5-a]pyridin-5-yl]-6-methyl-3-pyridyl]oxymethyl]cyclobutanecarbonitrile;
3-[[4-[2-[(2,6-dimethylpyrimidin-4-yl)amino]pyrazolo[1,5-a]pyridin-5-yl]-6-methyl-3-pyridyl]oxymethyl]cyclobutanecarbonitrile;
3-[[6-(2,2-difluoroethoxy)-4-[2-(pyridazin-3-ylamino)pyrazolo[1,5-a]pyridin-5-yl]-3-pyridyl]oxy]-2,2-dimethyl-propanenitrile;
3-[[6-(2,2-difluoroethoxy)-4-[2-(pyrazin-2-ylamino)pyrazolo[1,5-a]pyridin-5-yl]-3-pyridyl]oxy]-2,2-dimethyl-propanenitrile;
2-[[4-[2-[(2,6-dimethylpyrimidin-4-yl)amino]pyrazolo[1,5-a]pyridin-5-yl]-5-(4-hydroxycyclohex oxy)-2-pyridyl]oxy]acetonitrile;
3-[[4-[2-[(6-aminopyrazin-2-yl)amino]pyrazolo[1,5-a]pyridin-5-yl]-6-(difluoromethoxy)-3-pyrid yl]oxy]-2,2-dimethyl-propanenitrile;
3-[[6-(difluoromethoxy)-4-[2-[(1-methylpyrazol-4-yl)amino]pyrazolo[1,5-a]pyridin-5-yl]-3-pyridyl]oxy]-2,2-dimethyl-propanenitrile;
3-[[6-(2,2-difluoroethoxy)-4-[2-[(1,5-dimethyl-6-oxo-pyridazin-3-yl)amino]pyrazolo[1,5-a]pyrid in-5-yl]-3-pyridyl]oxy]-2,2-dimethyl-propanenitrile;
3-[[6-(difluoromethoxy)-4-[2-(3-pyridylamino)pyrazolo[1,5-a]pyridin-5-yl]-3-pyridyl]oxy]-2,2-dimethyl-propanenitrile;
3-[[6-(difluoromethoxy)-4-[2-[[5-(morpholinomethyl)-2-pyridyl]amino]pyrazolo[1,5-a]pyridin-5-yl]-3-pyridyl]oxy]-2,2-dimethyl-propanenitrile;
3-[[6-(2,2-difluoroethoxy)-4-[2-[(6-methylpyrazin-2-yl)amino]pyrazolo[1,5-a]pyridin-5-yl]-3-pyridyl]oxy]-2,2-dimethyl-propanenitrile;
3-[[6-(2,2-difluoroethoxy)-4-[2-[(1-methyl-6-oxo-pyridazin-3-yl)amino]pyrazolo[1,5-a]pyridin-5-yl]-3-pyridyl]oxy]-2,2-dimethyl-propanenitrile;
3-[[6-(difluoromethoxy)-4-[2-[(4-methoxypyrimidin-2-yl)amino]pyrazolo[1,5-a]pyridin-5-yl]-3-pyridyl]oxy]-2,2-dimethyl-propanenitrile;
3-[[6-(difluoromethoxy)-4-[2-[(1-methyl-1,2,4-triazol-3-yl)amino]pyrazolo[1,5-a]pyridin-5-yl]-3-pyridyl]oxy]-2,2-dimethyl-propanenitrile;
3-[[6-(difluoromethoxy)-4-[2-[[2-(ethylamino)-6-(trifluoromethyl)pyrimidin-4-yl]amino]pyrazolo[1,5-a]pyridin-5-yl]-3-pyridyl]oxy]-2,2-dimethyl-propanenitrile;
2-[[5-[5-(2-cyano-2-methyl-propoxy)-2-(difluoromethoxy)-4-pyridyl]pyrazolo[1,5-a]pyridin-2-yl]amino]-N-(2-hydroxy-2-methyl-propyl)-6-methyl-pyridine-4-carboxamide;
3-[[6-(difluoromethoxy)-4-[2-[(1-methyl-6-oxo-pyridazin-3-yl)amino]pyrazolo[1,5-a]pyridin-5-y 1]-3-pyridyl]oxy]-2,2-dimethyl-propanenitrile;
3-[[4-[2-[(2-tert-butyl-6-methyl-pyrimidin-4-yl)amino]pyrazolo[1,5-a]pyridin-5-yl]-6-(difluoromethoxy)-3-pyridyl]oxy]-2,2-dimethyl-propanenitrile;
3-[[4-[2-[(5-aminopyridazin-3-yl)amino]pyrazolo[1,5-a]pyridin-5-yl]-6-(difluoromethoxy)-3-pyridyl]oxy]-2,2-dimethyl-propanenitrile;
3-[[4-[2-[(2-aminopyrimidin-4-yl)amino]pyrazolo[1,5-a]pyridin-5-yl]-6-(difluoromethoxy)-3-pyridyl]oxy]-2,2-dimethyl-propanenitrile;
3-[[6-(difluoromethoxy)-4-[2-(4,5,6,7-tetrahydropyrazolo[1,5-a]pyrazin-2-ylamino)pyrazolo[1,5-a]pyridin-5-yl]-3-pyridyl]oxy]-2,2-dimethyl-propanenitrile;
3-[[6-(difluoromethoxy)-4-[2-(pyrazin-2-ylamino)pyrazolo[1,5-a]pyridin-5-yl]-3-pyridyl]oxy]-2,2-dimethyl-propanenitrile;
3-[[6-(difluoromethoxy)-4-[2-(pyridazin-3-ylamino)pyrazolo[1,5-a]pyridin-5-yl]-3-pyridyl]oxy]-2,2-dimethyl-propanenitrile;
3-[[6-(difluoromethoxy)-4-[2-[(5-methylpyrazin-2-yl)amino]pyrazolo[1,5-a]pyridin-5-yl]-3-pyridyl]oxy]-2,2-dimethyl-propanenitrile;
3-[[6-(difluoromethoxy)-4-[2-[(6-methyl-2-morpholino-pyrimidin-4-yl)amino]pyrazolo[1,5-a]pyridin-5-yl]-3-pyridyl]oxy]-2,2-dimethyl-propanenitrile;
3-[[6-(difluoromethoxy)-4-[2-[(6-methylpyridazin-3-yl)amino]pyrazolo[1,5-a]pyridin-5-yl]-3-pyridyl]oxy]-2,2-dimethyl-propanenitrile;
3-[[6-(difluoromethoxy)-4-[2-[[6-(1-hydroxy-1-methyl-ethyl)pyridazin-3-yl]amino]pyrazolo[1,5-a]pyridin-5-yl]-3-pyridyl]oxy]-2,2-dimethyl-propanenitrile;
3-[[6-(difluoromethoxy)-4-[2-[[5-(1-hydroxy-1-methyl-ethyl)pyrazin-2-yl]amino]pyrazolo[1,5-a]pyridin-5-yl]-3-pyridyl]oxy]-2,2-dimethyl-propanenitrile;
3-[[6-(difluoromethoxy)-4-[2-[(1-methylpyrazol-3-yl)amino]pyrazolo[1,5-a]pyridin-5-yl]-3-pyridyl]oxy]-2,2-dimethyl-propanenitrile;
2-[[5-[5-(2-cyano-2-methyl-propoxy)-2-(difluoromethoxy)-4-pyridyl]pyrazolo[1,5-a]pyridin-2-yl]amino]-N,6-dimethyl-pyridine-4-carboxamide;
3-[[6-(difluoromethoxy)-4-[2-(5,6,7,8-tetrahydro-2,7-naphthyridin-3-ylamino)pyrazolo[1,5-a]pyridin-5-yl]-3-pyridyl]oxy]-2,2-dimethyl-propanenitrile;
3-[[6-(difluoromethoxy)-4-[2-[(2-methylpyrimidin-4-yl)amino]pyrazolo[1,5-a]pyridin-5-yl]-3-pyridyl]oxy]-2,2-dimethyl-propanenitrile;
3-[[6-(difluoromethoxy)-4-[2-(5,6,7,8-tetrahydro-2,6-naphthyridin-3-ylamino)pyrazolo[1,5-a]pyridin-5-yl]-3-pyridyl]oxy]-2,2-dimethyl-propanenitrile;
3-[[6-(difluoromethoxy)-4-[2-(5,6,7,8-tetrahydro-1,7-naphthyridin-2-ylamino)pyrazolo[1,5-a]pyridin-5-yl]-3-pyridyl]oxy]-2,2-dimethyl-propanenitrile;
1-[[6-(difluoromethoxy)-4-[2-[(2,6-dimethylpyrimidin-4-yl)amino]pyrazolo[1,5-a]pyridin-5-yl]-3-pyridyl]oxymethyl]cyclopropanecarbonitrile;
(*R)-2-[[4-[2-[(2,6-dimethylpyrimidin-4-yl)amino]pyrazolo[1,5-a]pyridin-5-yl]-5-(4-hydroxycyclohexoxy)-2-pyridyl]oxy]propanenitrile;
3-[2-[[4-[2-[(2,6-dimethylpyrimidin-4-yl)amino]pyrazolo[1,5-a]pyridin-5-yl]-6-methyl-3-pyridyl]oxy]ethyl]oxetane-3-carbonitrile;
N-[5-[2-(difluoromethoxy)-5-(4-hydroxycyclohexoxy)-4-pyridyl]pyrazolo[1,5-a]pyridin-2-yl]cyclopropanecarboxamide;
N-[5-[2-(cyanomethoxy)-5-(2-cyano-2-methyl-propoxy)-4-pyridyl]pyrazolo[1,5-a]pyridin-2-yl]cyclopropanecarboxamide;
3-[[6-(2,2-difluoroethoxy)-4-[2-[[6-methyl-2-(methylamino)pyrimidin-4-yl]amino]pyrazolo[1,5-a]pyridin-5-yl]-3-pyridyl]oxy]-2,2-dimethyl-propanenitrile;
(*S)—N-[5-[2-(1-cyanoethoxy)-5-(2-cyano-2-methyl-propoxy)-4-pyridyl]pyrazolo[1,5-a]pyridin-2-yl]cyclopropanecarboxamide;
3-[[6-(difluoromethoxy)-4-[2-[(2,6-dimethylpyrimidin-4-yl)amino]pyrazolo[1,5-a]pyridin-5-yl]-3-pyridyl]oxy]-2,2-dimethyl-propanamide;
3-[[6-(difluoromethoxy)-4-[2-[(2,6-dimethylpyrimidin-4-yl)amino]pyrazolo[1,5-a]pyridin-5-yl]-3-pyridyl]oxy]-N,N,2,2-tetramethyl-propanamide;
3-[[6-(difluoromethoxy)-4-[2-[(2,6-dimethylpyrimidin-4-yl)amino]imidazo[1,2-a]pyrazin-6-yl]-3-pyridyl]oxy]-2,2-dimethyl-propanenitrile;
3-((4-(2-((1-cyclopropyl-5-methyl-6-oxo-1,6-dihydropyridazin-3-yl)amino)pyrazolo[1,5-a]pyridin-5-yl)-6-(2,2-difluoroethoxy)pyridin-3-yl)oxy)-2,2-dimethylpropanenitrile;
(*R)-4-[[6-(difluoromethoxy)-4-[2-[(2,6-dimethylpyrimidin-4-yl)amino]pyrazolo[1,5-a]pyridin-5-yl]-3-pyridyl]oxymethyl]-5,5-dimethyl-pyrrolidin-2-one;
(*S)-4-[[6-(difluoromethoxy)-4-[2-[(2,6-dimethylpyrimidin-4-yl)amino]pyrazolo[1,5-a]pyridin-5-yl]-3-pyridyl]oxymethyl]-5,5-dimethyl-pyrrolidin-2-one;
5-[2-(difluoromethoxy)-5-(2-methyl-2-methylsulfonyl-propoxy)-4-pyridyl]-N-(2,6-dimethylpyrimidin-4-yl)pyrazolo[1,5-a]pyridin-2-amine;
(*S)-4-[[6-(difluoromethoxy)-4-[2-[(2,6-dimethylpyrimidin-4-yl)amino]pyrazolo[1,5-a]pyridin-5-yl]-3-pyridyl]oxymethyl]-1,5,5-trimethyl-pyrrolidin-2-one;
N-[5-(2-methoxyphenyl)pyrazolo[1,5-a]pyridin-2-yl]cyclopropanecarboxamide;
N-[5-(2-ethoxyphenyl)pyrazolo[1,5-a]pyridin-2-yl]cyclopropanecarboxamide;
N-[5-(2-methoxy-5-methyl-phenyl)pyrazolo[1,5-a]pyridin-2-yl]cyclopropanecarboxamide;
N-[5-(2-methoxy-4-methyl-phenyl)pyrazolo[1,5-a]pyridin-2-yl]cyclopropanecarboxamide;
N-[5-(2-methoxy-6-methyl-phenyl)pyrazolo[1,5-a]pyridin-2-yl]cyclopropanecarboxamide;
N-[5-(2-methoxy-3-methyl-phenyl)pyrazolo[1,5-a]pyridin-2-yl]cyclopropanecarboxamide;
N-[5-(2-methoxy-3-pyridyl)pyrazolo[1,5-a]pyridin-2-yl]cyclopropanecarboxamide;
N-[5-(3-methoxy-4-pyridyl)pyrazolo[1,5-a]pyridin-2-yl]cyclopropanecarboxamide;
N-[5-(3-methoxy-2-pyridyl)pyrazolo[1,5-a]pyridin-2-yl]cyclopropanecarboxamide;
N-[5-(4-methoxy-3-pyridyl)pyrazolo[1,5-a]pyridin-2-yl]cyclopropanecarboxamide;
N-[5-(5-ethoxy-2-methyl-4-pyridyl)pyrazolo[1,5-a]pyridin-2-yl]cyclopropanecarboxamide;
N-[5-(5-benzyloxy-2-cyano-4-pyridyl)pyrazolo[1,5-a]pyridin-2-yl]cyclopropanecarboxamide;
3-[2-[2-[(2,6-dimethylpyrimidin-4-yl)amino]pyrazolo[1,5-a]pyridin-5-yl]-4-(trifluoromethoxy)phenoxy]-2,2-dimethyl-propanenitrile;
(*R)-5-[5-(2-amino-3,3,3-trifluoro-2-methyl-propoxy)-2-(di fluoromethoxy)-4-pyridyl]-N-(2,6-dimethylpyrimidin-4-yl)pyrazolo[1,5-a]pyridin-2-amine; and
(*S)-5-[5-(2-amino-3,3,3-trifluoro-2-methyl-propoxy)-2-(di fluoromethoxy)-4-pyridyl]-N-(2,6-dimethylpyrimidin-4-yl)pyrazolo[1,5-a]pyridin-2-amine;
or a tautomer, stereoisomer pharmaceutically acceptable salt, or solvate thereof.

18. A compound selected from the group consisting of:
N-[5-(2-methoxy-4-methyl-phenyl)pyrazolo[1,5-a]pyridin-2-yl]cyclopropanecarboxamide;
N-[5-(2-methoxy-6-methyl-phenyl)pyrazolo[1,5-a]pyridin-2-yl]cyclopropanecarboxamide;
N-[5-(2-methoxy-3-methyl-phenyl)pyrazolo[1,5-a]pyridin-2-yl]cyclopropanecarboxamide;
N-[5-(3-methoxy-2-pyridyl)pyrazolo[1,5-a]pyridin-2-yl]cyclopropanecarboxamide; and
N-[5-(4-methoxy-3-pyridyl)pyrazolo[1,5-a]pyridin-2-yl]cyclopropanecarboxamide;
or a tautomer, stereoisomer pharmaceutically acceptable salt, or solvate thereof.

19. A pharmaceutical composition comprising the compound of claim 1, and a pharmaceutically acceptable carrier.

20. A method of inhibiting a salt inducible kinase (SIK) in a subject in need thereof, the method comprising administering to the subject the pharmaceutical composition of claim 19.

21. The method of claim 20, wherein the subject is in need of a treatment of a disease, disorder, or condition mediated by a SIK, such as an autoimmune disorder or a proliferative disorder.
